(19) United States
(12) Patent Application Publication
(10) Pub. No.: US 2025/0346585 A1
(43) Pub. Date: Nov. 13, 2025

(54) PHARMACEUTICAL COMPOUNDS FOR THE TREATMENT OF COMPLEMENT MEDIATED DISORDERS

(71) Applicant: Alexion Pharmaceuticals, Inc., Boston, MA (US)

(72) Inventors: Venkat Rao Gadhachanda, Hamden, CT (US); Jason Allan Wiles, Madison, CT (US); Evans Onyango, Hamden, CT (US)

(73) Assignee: Alexion Pharmaceuticals, Inc., Boston, MA (US)

(21) Appl. No.: 18/847,908

(22) PCT Filed: Mar. 22, 2023
(86) PCT No.: PCT/US2023/015932

§ 371 Date: Sep. 17, 2024

Related U.S. Application Data
(60) Provisional application No. 63/322,554, filed on Mar. 22, 2022.

(51) Int. Cl. C07D 409/14; C07D 409/12; C07D 411/14; C07D 417/14; C07D 487/10; C07D 491/113

(52) U.S. Cl. CPC C07D 409/14; C07D 409/12; C07D 411/14; C07D 417/14; C07D 487/10; C07D 491/113

(57) ABSTRACT

This disclosure provides compounds, compositions, and methods to treat medical disorders, such as complement-mediated disorders, including complement C1s-mediated disorders.

### FIELD OF THE DISCLOSURE

Herein are provided pharmaceutical compounds to treat medical disorders, such as complement-mediated disorders, including complement C1-mediated disorders.

### BACKGROUND OF THE DISCLOSURE

The complement system is a part of the innate immune system which does not adapt to changes over the course of the subject's life but is recruited and used by the adaptive immune system. For example, it assists, or complements, the ability of antibodies and phagocytic cells to clear pathogens. This sophisticated regulatory pathway allows rapid reaction to pathogenic organisms while protecting host cells from destruction. Over thirty proteins and protein fragments make up the complement system. These proteins act through opsonization (enhancing phagocytosis of antigens), chemotaxis (attracting macrophages and neutrophils), cell lysis (rupturing membranes of foreign cells), and agglutination (clustering and binding of pathogens together).

The complement system has three pathways: classical, alternative, and lectin. The classical pathway is triggered by antibody-antigen complexes with the antibody isotypes IgG and IgM. The antibody-antigen complex binds to C1 and this initiates the cleavage of C4 and C2 to generate C3 convertase that then splits C3 into C3a and C3b. C3a interacts with its C3a receptor to recruit leukocytes, while C3b binds to C3 convertase to form C5 convertase. C5 convertase cleaves C5 into C5a and C5b. Similar to C3a, C5a interacts with its C5a receptor to recruit leukocytes, but C5b interacts with C6, C7, C8, and C8 and together these proteins form the cylindrical membrane attack complex (MAC) that causes the cell to swell and burst. These immune responses can be inhibited by preventing C1 from being able to bind the antibody-antigen complex.

Given the range of serious diseases mediated by a disfunction of the complement system, there is a clear medical need to provide pharmaceutically acceptable compounds, methods, compositions, and methods of manufacture to inhibit the complement system in a patient in need thereof.

Therefore, the present disclosure provides compounds and their uses and compositions to treat disorders arising from or amplified by a disfunction of the complement system. The present disclosure also provides compounds, uses, compositions, combinations, and processes of manufacture that inhibit C1s (complement C1 esterase) and thus can treat disorders mediated by C1s.

### SUMMARY

The present disclosure provides compounds, compositions, and methods for treating a disorder mediated by the complement cascade (including a dysfunctional cascade), a disorder or abdormality of a cell that adversely affects the the ability of the cell to engage in or respond to normal complement activity including for example, the classical complement pathway, or an undesired complement-mediated response to a medical treatment, such as surgery or other medical procedure or a pharmaceutical or biopharmaceutical drug administration, a blood transfusion, or other allogenic tissue or fluid administration. In some embodiments, the active compound may act as an inhibitor of the complement classical pathway by inhibiting complement C1s.

Without wishing to be bound by theory, the present disclosure is based, in part, on the unexpected discovery that compounds of the disclosure exhibit advantageous properties over structurally related compounds (e.g., the compounds described in WO 2020/198062), such as improved C1s inhibiting activity, improved classical pathway hemolysis inhibiting activity, improved bioavailability, and/or improved metabolic stability. In some embodiments, compounds of the present disclosure may function as prodrugs.

In one embodiment, the disorder is associated with the complement classical pathway and the compound inhibits the classical pathway. In yet another embodiment, the disorder is associated with the alternative complement cascade pathway. In a further embodiment, the disorder is associated with the complement lectin pathway. Alternatively, the active compound or its salt may act through a different mechanism of action than the complement cascade to treat a disorder described herein. In another embodiment, the active compound, and/or its salt, inhibits a combination of these pathways.

In one aspect, the present disclosure provides a compound of formula (I):

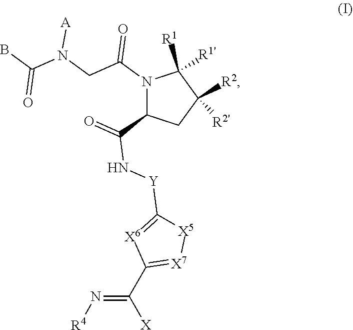

or a pharmaceutically acceptable salt thereof, in which each of R¹, R^{1′}, R², and R^{2′} is independently selected from H; halo; optionally substituted C₁-C₅ alkyl; optionally substituted C₃-C₈ cycloalkyl; optionally substituted C₆-C₁₄ aryl; optionally substituted 5- to 10-membered heterocycle; optionally substituted 5- to 10-membered heteroaryl; optionally substituted C₁-C₆ alkoxy; optionally substituted C₆-C₁₄ aryloxy; SO₂R^{a}, in which R^{a} is H, C₁-C₆ alkyl, or C₃-C₈ cycloalkyl; and S(O)(NH)R^{b}, in which R^{b} is H or C₁-C₅ alkyl; in which at least one of R¹, R^{1′}, R², and R^{2′} is not H; or R¹ and R², together with the atoms to which each is attached, form optionally substituted C₃-C₈ cycloalkyl; or R² and R^{2′}, together with the atom to which they are attached, form optionally substituted 5- or 6-membered spirocyclic heterocycle; or R² and R^{2′} combine to form ═C(R^{c})₂, in which each R^{c} is independently H or halo; Y is selected from

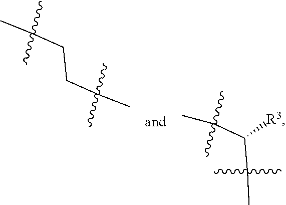

in which R³ is H, CH₃, or CF₃; X is N(R^{d})₂, in which each R^{d} is independently H; OH, OC(O)(C₁-C₆ alkyl); and R⁴ is H or C(O)OR^{e}, in which R^{e} is C₁-C₆ alkyl or C₆-C₁₄ aryl; or X and R⁴, together with the atoms to which each is attached, form 1H-imidazole-2-yl or (5-aminothiazol-2-yl)thiopheny-2-yl; A is H or C₁-C₆ alkyl; B is selected from:

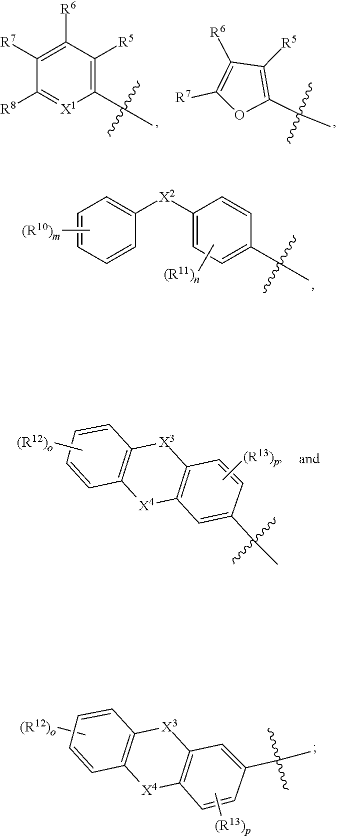

X¹ is CR⁹ or N; each of R⁵, R⁶, and R⁹ is independently selected from H, halo, and optionally substituted C₁-C₆ alkyl, or R⁵ and A combine to form optionally substituted C₁-C₂ alkylene; one of R⁷ and R⁸ is optionally substituted C₆-C₁₄ aryl or optionally substituted 5- to 10-membered heteroaryl; and the other is H, halo, or optionally substituted C₁-C₆ alkyl; or R⁷ and R⁸, together with the atoms to which each is attached; form optionally substituted 5- or 6-membered heterocycle, optionally substituted 5- to 10-membered heteroaryl, or optionally substituted C₆-C₁₄ aryl; or R⁶ and R⁹ combine to form (C₂-C₆alkylene)(C₆-C₁₄arylene)(C₂-C₆alkylene), and each of R₅, R₇, and R₈ is H; X² is O or C(R^{f})₂, in which each R^{f} is halo; m is selected from 0, 1, 2, 3, 4, and 5; n is selected from 0, 1, 2, 3, and 4; each R¹⁰ and R¹¹ is independently halo, CN, optionally substituted C₁-C₆ alkyl, optionally substituted C₁-C₆ alkoxy, or optionally substituted C₃-C₈ cycloalkyl; each of X³ and X⁴ is a bond; O; S; C(R^{g})₂, in which each R^{g} is independently H, OH, halo, optionally substituted C₁-C₆ alkyl, or optionally substituted C₁-C₆ alkoxy, or both R^{g} combine to form oxo; NR^{h}, in which R^{h} is H or C₁-C₆ alkyl; or SO₂; X⁵ is S, and X⁶ and X⁷ are both CH; or X⁶ is S, and X⁵ and X⁷ are both CH; or X⁷ is S, and X⁵ and X⁶ are both CH; o is selected from 0, 1, 2, 3, and 4; p is selected from 0, 1, 2, and 3; and each R¹² and R¹³ is independently halo, optionally substituted C₁-C₆ alkyl, optionally substituted C₁-C₆ alkoxy, or optionally substituted C₃-C₈ cycloalkyl, provided that when B is

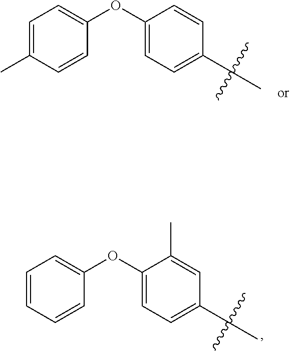 [SMILES: *c1ccc(Oc2ccc(C)cc2)cc1 ; *c1ccc(Oc2ccccc2)c(C)c1]

R³ is CH₃, CF₃, or CH₂OH; when m is 0, n is 1, 2, 3, or 4; and when n is 0, m is 1, 2, 3, 4, or 5.

In some embodiments, the compound is a compound of formula (II):

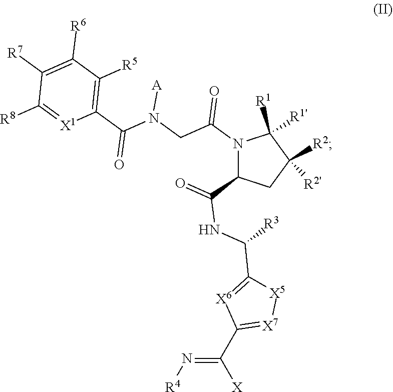

or a pharmaceutically acceptable salt thereof.

In some embodiments, X¹ is N.

In some embodiments, X¹ is CR⁹, e.g., CH.

In some embodiments, in which X¹ is CR⁹, R⁶ and R⁹ combine to form (C₂-C₆alkylene)(C₆-C₁₄arylene)(C₂-C₆alkylene), e.g.,

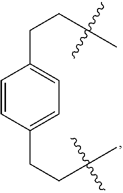 [SMILES: *CCc1ccc(CC*)cc1]

and each of R₅, R₇, and R₈ is H.

In some embodiments, R⁷ is optionally substituted C₆-C₁₄ aryl or optionally substituted 5- to 10-membered heteroaryl.

In some embodiments, R⁷ is optionally substituted C₆-C₁₄ aryl, such as optionally substituted phenyl. For example, R⁷ may be any one of:

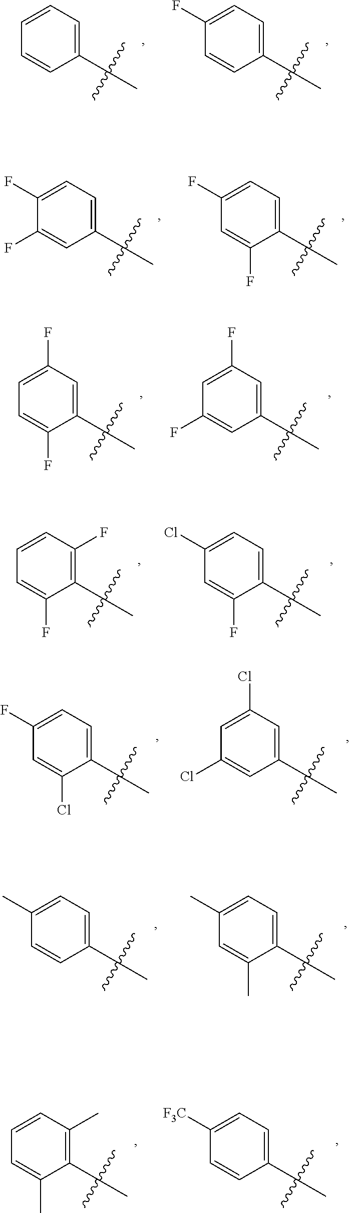

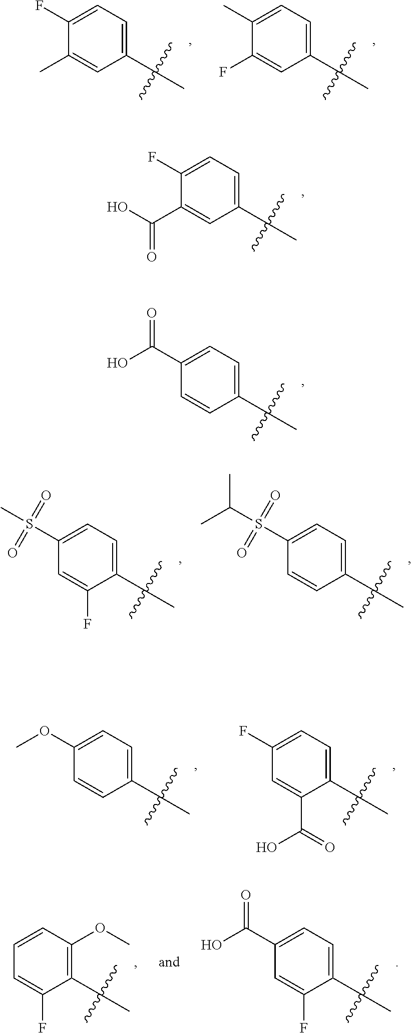

In some embodiments, R⁷ is optionally substituted 5- to 10-membered heteroaryl. For example, R⁷ may be any one of:

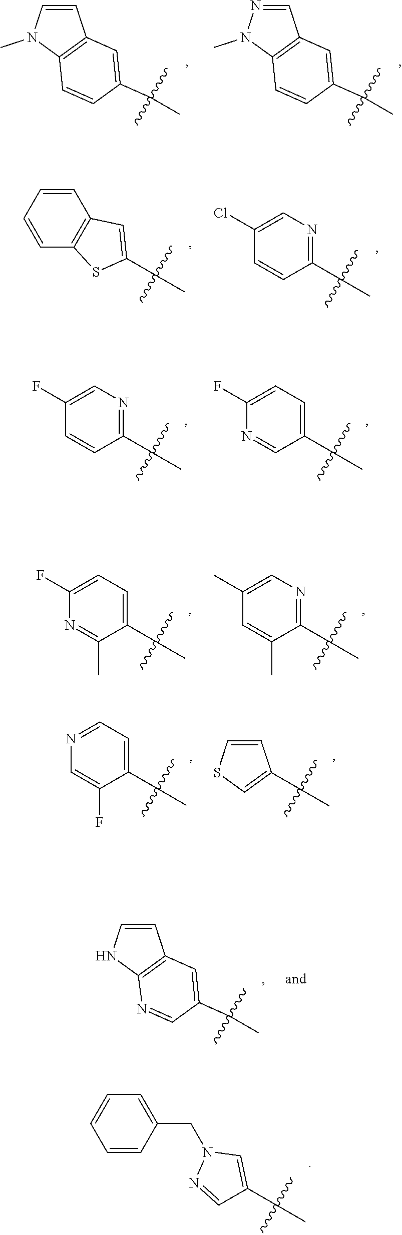

In some embodiments, in which R⁷ is optionally substituted C₆-C₁₄ aryl or optionally substituted 5- to 10-membered heteroaryl, R⁸ is H.

In some embodiments, in which R⁷ is optionally substituted C₆-C₁₄ aryl or optionally substituted 5- to 10-membered heteroaryl, R⁸ is optionally substituted C₁-C₆ alkyl, e.g., CH₃.

In some embodiments, in which R⁷ is optionally substituted C₆-C₁₄ aryl or optionally substituted 5- to 10-membered heteroaryl, R⁸ is halo, e.g., F.

In some embodiments, R⁸ is optionally substituted C₆-C₁₄ aryl or optionally substituted 5- to 10-membered heteroaryl.

In some embodiments, R⁸ is optionally substituted C₆-C₁₄ aryl, e.g., optionally substituted phenyl. For example, R⁸ may be:

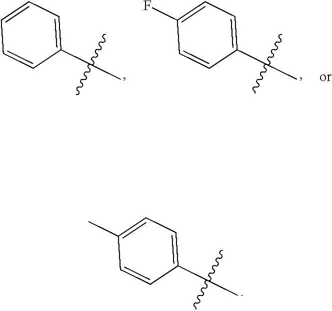

In some embodiments, R⁸ is optionally substituted 5- to 10-membered heteroaryl.

In some embodiments, in which R⁸ is optionally substituted C₆-C₁₄ aryl or optionally substituted 5- to 10-membered heteroaryl, R⁷ is H.

In some embodiments, in which R⁸ is optionally substituted C₆-C₁₄ aryl or optionally substituted 5- to 10-membered heteroaryl, R⁷ is halo, e.g., F.

In some embodiments, R⁷ and R⁸, together with the atoms to which each is attached; form optionally substituted 5- or 6-membered heterocycle, 5- to 10-membered heteroaryl, or optionally substituted C₆-C₁₄ aryl.

In some embodiments, R⁷ and R⁸, together with the atoms to which each is attached, form optionally substituted 5- or 6-membered heterocycle, e.g.,

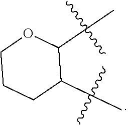 [SMILES: *C1CCCOC1*]

In some embodiments, R⁷ and R⁸, together with the atoms to which each is attached, form optionally substituted C₆-C₁₄ aryl. In some embodiments, R⁷ and R⁸, together with the atoms to which each is attached, form

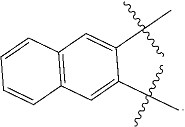 [SMILES: *c1cc2ccccc2cc1*]

In some embodiments, R⁷ and R³, together with the atoms to which each is attached, form

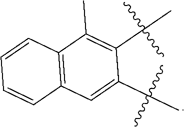 [SMILES: *c1cc2ccccc2c(C)c1*]

In some embodiments, R⁷ and R⁸, together with the atoms to which each is attached, form optionally substituted 5- to 10-membered heteroaryl. In some embodiments, R⁷ and R⁸, together with the atoms to which each is attached, form

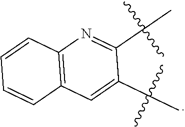 [SMILES: *c1cc2ccccc2nc1*]

In some embodiments, R⁷ and R⁸, together with the atoms to which each is attached, form

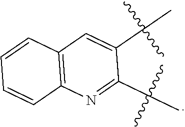 [SMILES: *c1cc2ccccc2nc1*]

In some embodiments, R⁶ is H.

In some embodiments, R⁵ is H. In some embodiments, R⁵ is halo, e.g., F. In some embodiments, R⁵ and A combine to form optionally substituted C₁-C₂ alkylene. In some embodiments, R⁵ and A combine to form —CH₂—. In some embodiments, R⁵ and A combine to form —(CH₂)₂—.

In some embodiments, the compound is a compound of formula (III):

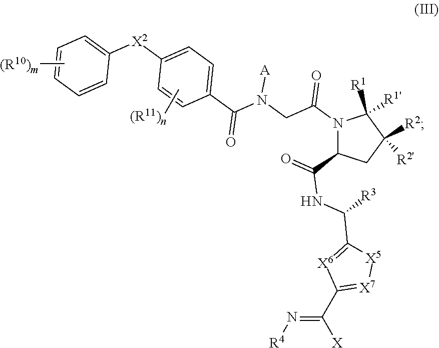

or a pharmaceutically acceptable salt thereof, e.g., a compound of formula (IIIa):

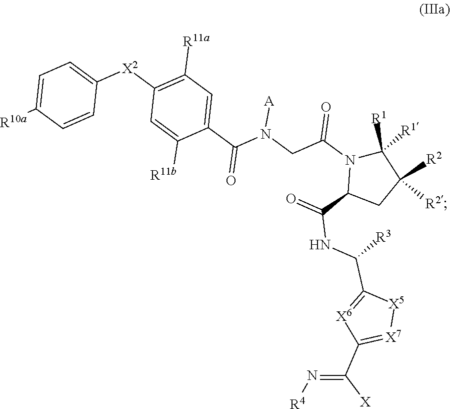

or a pharmaceutically acceptable salt thereof, in which each of R^{10a}, R^{11a} and R^{11b} is independently H, halo, cyano, optionally substituted C₁-C₆alkyl, optionally substituted C₁-C₆ alkoxy, or optionally substituted C₃-C₈ cycloalkyl.

In some embodiments, X² is O. In some embodiments, X² is C(R^{f})₂, e.g., CF₂.

In some embodiments, R^{10a} is optionally substituted C₁-C₆ alkyl. In some embodiments, R^{10a} is CH₃. In some embodiments, R^{10a} is CH₂CH₃. In some embodiments, R^{10a} is CH(CH₃)₂. In some embodiments, R^{10a} is CH(CH₂F)₂. In some embodiments, R^{10a} is halo. In some embodiments, R^{10a} is F. In some embodiments, R^{10a} is Cl. In some embodiments, R^{10a} is optionally substituted C₃-C₈ cycloalkyl, e.g., cyclopropyl. In some embodiments, R^{10a} is CN.

In some embodiments, R^{11a} is H. In some embodiments, R^{11a} is optionally substituted C₁-C₆ alkyl, e.g., CH₃.

In some embodiments, R^{11b} is H. In some embodiments, R^{11b} is optionally substituted C₁-C₆ alkyl, e.g., CH₃. In some embodiments, R^{11b} is halo, e.g., F.

In some embodiments, R^{10a} is H. In some embodiments, in which R^{10a} is H, R^{11a} is H. In some embodiments, in which R^{10a} is H, R^{11a} is optionally substituted C₁-C₆ alkyl, e.g., CH₃. In some embodiments, in which R^{10a} is H and R^{11a} is optionally substituted C₁-C₆ alkyl (e.g., CH₃), R^{11b} is H. In some embodiments, in which R^{10a} is H, R^{11b} is optionally substituted C₁-C₆ alkyl, e.g., CH₃. In some embodiments, in which R^{10a} is H, R^{11b} is halo, e.g., F. In some embodiments, in which X² is C(R^{f})₂, each of R^{10a}, R^{11a}, and R^{11b} is H.

In some embodiments, the compound is a compound of formula (IV):

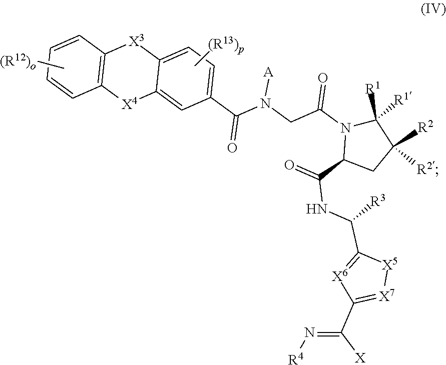

or a pharmaceutically acceptable salt thereof, e.g., a compound of formula (IVa):

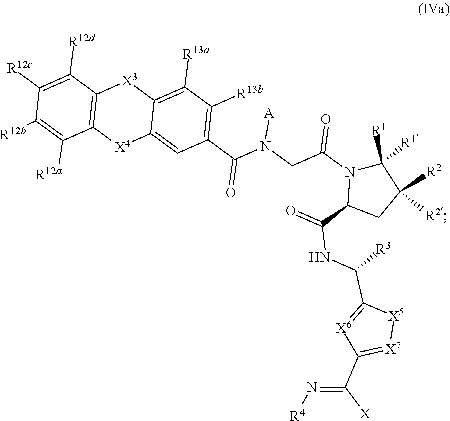

or a pharmaceutically acceptable salt thereof, in which each of R^{12a}, R^{12b}, R^{12c}, R^{12d}, R^{13a} and R^{13b} is independently H, halo, optionally substituted C₁-C₆ alkyl, optionally substituted C₁-C₆ alkoxy, or optionally substituted C₃-C₈ cycloalkyl.

In some embodiments, R^{12a} is H. In some embodiments, R^{12a} is optionally substituted C₁-C₆ alkyl, e.g., CH₃. In some embodiments, R^{12b} is H. In some embodiments, R^{12b} is optionally substituted C₁-C₆ alkyl, e.g., CH₃. In some embodiments, R^{12c} is H. In some embodiments, R^{12c} is optionally substituted C₁-C₆ alkyl, e.g., CH₃. In some embodiments, R^{12c} is optionally substituted C₁-C₆ alkoxy, e.g., OCH₃. In some embodiments, R^{12c} is halo, e.g., F or Cl. In some embodiments, R^{12d} is H. In some embodiments, R^{12d} is optionally substituted C₁-C₆ alkyl, e.g., CH₃. In some embodiments, R^{13a} is H. In some embodiments, R^{13a} is optionally substituted C₁-C₆ alkyl, e.g., CH₃. In some embodiments, R^{13b} is H. In some embodiments, R^{13b} is optionally substituted C₁-C₆ alkyl, e.g., CH₃.

In some embodiments, the compound is a compound of formula (V):

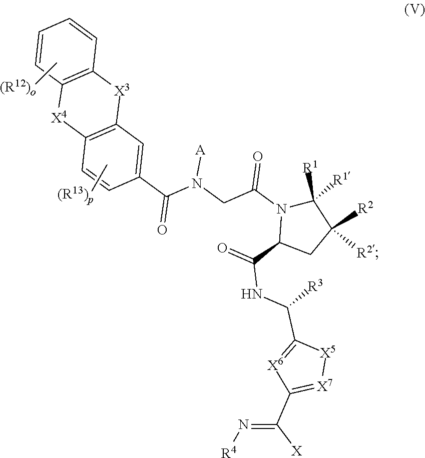

or a pharmaceutically acceptable salt thereof.

In some embodiments, in which the compound is a compound of formula (V), o is 0.

In some embodiments, in which the compound is a compound of formula (V), p is 0.

In some embodiments, X³ is a bond.

In some embodiments, X³ is O. In some embodiments, X³ is C(R⁹)₂. In some embodiments, X³ is CF₂. In some embodiments, X³ is CH₂. In some embodiments, X³ is CHCH₃. In some embodiments, X³ is C(CH₃)₂. In some embodiments, X³ is C(O). In some embodiments, X³ is CHF. In some embodiments, X³ is CHOH. In some embodiments, X³ is CHCHF₂. In some embodiments, X³ is CHOCHF₂. In some embodiments, X³ is CHOCH₃. In some embodiments, X³ is NR^{h}, e.g., NH. In some embodiments, X³ is S. In some embodiments, X³ is SO₂.

In some embodiments, X⁴ is a bond. In some embodiments, X⁴ is O. In some embodiments, X⁴ is S.

In some embodiments, the compound is a compound of formula (VI):

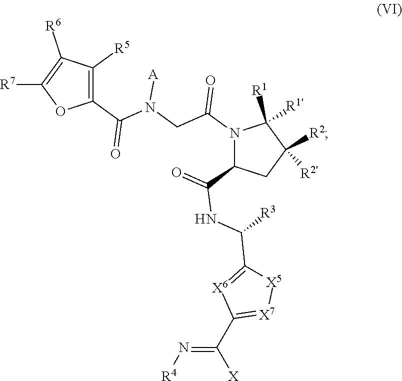

or a pharmaceutically acceptable salt thereof.

In some embodiments, in which the compound is a compound of formula (VI), R⁵ is H. In some embodiments, in which the compound is a compound of formula (VI), R⁶ is H. In some embodiments, in which the compound is a compound of formula (VI), R⁷ is optionally substituted C₆-C₁₄ aryl, e.g., optionally substituted phenyl. For example, R⁷ may be any one of

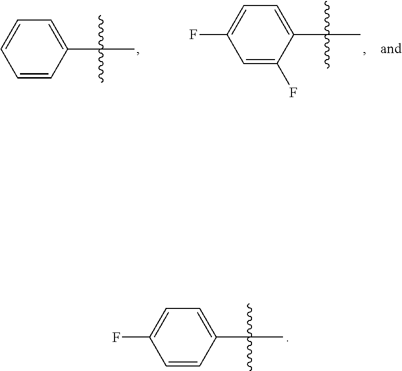 [SMILES: *c1ccccc1 ; *c1ccc(F)cc1F ; *c1ccc(F)cc1]

In some embodiment in any of the aspects disclosed herein (e.g., any one of formulas (I), (II), (III), (IIIa), (IV), (IVa), (V), and (VI)), R¹ and R², together with the atoms to which each is attached, combine to form optionally substituted C₃-C₈ cycloalkyl, e.g., cyclopropyl.

In some embodiment in any of the aspects disclosed herein (e.g., any one of formulas (I), (II), (III), (IIIa), (IV), (IVa), (V), and (VI)), in which R¹ and R², together with the atoms to which each is attached, combine to form optionally substituted C₃-C₈ cycloalkyl (e.g., cyclopropyl), R^{1′} is H.

In some embodiment in any of the aspects disclosed herein (e.g., any one of formulas (I), (II), (III), (IIIa), (IV), (IVa), (V), and (VI)), in which R¹ and R², together with the atoms to which each is attached, combine to form optionally substituted C₃-C₈ cycloalkyl (e.g., cyclopropyl), R^{2′} is optionally substituted C₁-C₆ alkyl. In some embodiments, R^{2′} is C₁-C₆ alkyl substituted with one or more substituents independently selected from halo, hydroxy, NH₂, N(CH₃)₂, optionally substituted C₁-C₆ alkoxy, 5- or 6-membered heteroaryl, or 5- or 6-memebered heterocycle. In some embodiments, R^{2′} is CH₃. In some embodiments, R^{2′} is CH₂OH. In some embodiments, R^{2′} is CH₂OCH₃. In some embodiments, R^{2′} is CH₂CF₃. In some embodiments, R^{2′} is CH₂O(CH₂)₂NH₂. In some embodiments, R^{2′} is CH₂O(CH₂)₂N(CH₃)₂. In some embodiments, R^{2′} is CH₂O(CH₂)₃NH₂. In some embodiments, R^{2′} is CH₂O(CH₂)₃N(CH₃)₂. In some embodiments, R^{2′} is CH₂O(CH₂)₄NH₂. In some embodiments, R^{2′} is CH₂O(CH₂)₄N(CH₃)₂. In some embodiments, R^{2′} is CH₂NH₂. In some embodiments, R^{2′} is CH₂N(CH₃)₂. In some embodiments, R^{2′} is CH₂NH(CH₂)₂NHC(O)CH₃. In some embodiments, R^{2′} is

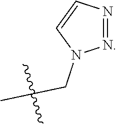 [SMILES: *Cn1ccnn1]

In some embodiments, R^{2′} is

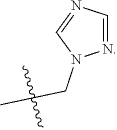 [SMILES: *Cn1cncn1]

In some embodiments, R^{2′} is

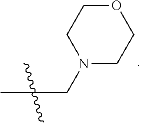 [SMILES: *CN1CCOCC1]

In some embodiments, R^{2′} is

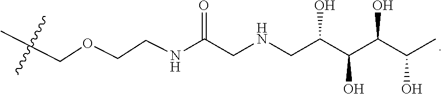 [SMILES: *COCCNC(=O)CNC[C@H](O)[C@H](O)[C@@H](O)[C@H](C)O]

In some embodiment in any of the aspects disclosed herein (e.g., any one of formulas (I), (II), (III), (IIIa), (IV), (IVa), (V), and (VI)), R² and R^{2′}, together with the atom to which they are attached, form optionally substituted 5- or 6-membered spirocyclic heterocycle. In some embodiments, R² and R^{2′}, together with the atom to which they are attached, form

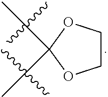 [SMILES: *C1(*)OCCO1]

In some embodiments, R² and R^{2′}, together with the atom to which they are attached, form

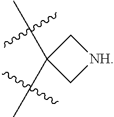 [SMILES: *C1(*)CNC1]

In some embodiment in any of the aspects disclosed herein (e.g., any one of formulas (I), (II), (III), (IIIa), (IV), (IVa), (V), and (VI)), R² is H.

In some embodiment in any of the aspects disclosed herein (e.g., any one of formulas (I), (II), (III), (IIIa), (IV), (IVa), (V), and (VI)), R² is optionally substituted C₁-C₆ alkyl. In some embodiments, R² is CH₂F. In some embodiments, R² is CH₂OH. In some embodiments, R² is CH₂OCH₃. In some embodiments, R² is CH₂O(CH₂)₅COOH. In some embodiments, R² is CH₂O(CH₂)₇COOH. In some embodiments, R² is CH₂O(CH₂)₉COOH. In some embodiments, R² is

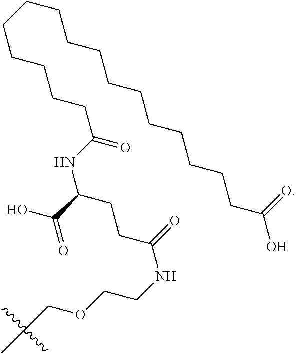 [SMILES: *COCCNC(=O)CC[C@H](NC(=O)CCCCCCCCCCCCCCCCC(=O)O)C(=O)O]

In some embodiments, R² is CH₂NH₂. In some embodiments, R² is CH₂N(CH₃)₂. In some embodiments, R² is CH₃. In some embodiments, R² is

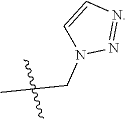 [SMILES: *Cn1ccnn1]

In some embodiments, R² is

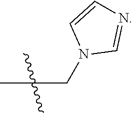 [SMILES: *Cn1ccnc1]

In some embodiment in any of the aspects disclosed herein (e.g., any one of formulas (I), (II), (III), (IIIa), (IV), (IVa), (V), and (VI)), R² is optionally substituted C₆-C₁₄ aryl, e.g.,

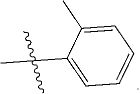 [SMILES: *c1ccccc1C]

In some embodiment in any of the aspects disclosed herein (e.g., any one of formulas (I), (II), (III), (IIIa), (IV), (IVa), (V), and (VI)), R² is optionally substituted C₃-C₈ cycloalkyl, e.g., cyclohexyl.

In some embodiment in any of the aspects disclosed herein (e.g., any one of formulas (I), (II), (III), (IIIa), (IV), (IVa), (V), and (VI)), R² is optionally substituted 5- to 10-membered heterocycle, e.g.,

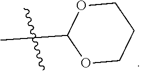 [SMILES: *C1OCCCO1]

In some embodiment in any of the aspects disclosed herein (e.g., any one of formulas (I), (II), (III), (IIIa), (IV), (IVa), (V), and (VI)), R² is optionally substituted 5- to 10-membered heteroaryl. In some embodiments, R² is

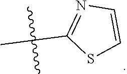 [SMILES: *c1nccs1]

In some embodiments, R² is

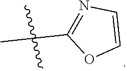 [SMILES: *c1ncco1]

In some embodiments, R² is

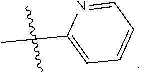 [SMILES: *c1ccccn1]

In some embodiments, R² is

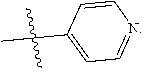 [SMILES: *c1ccncc1]

In some embodiment in any of the aspects disclosed herein (e.g., any one of formulas (I), (II), (III), (IIIa), (IV), (IVa), (V), and (VI)), R^{2′} is H.

In some embodiment in any of the aspects disclosed herein (e.g., any one of formulas (I), (II), (III), (IIIa), (IV), (IVa), (V), and (VI)), R^{2′} is optionally substituted C₁-C₆ alkyl. In some embodiments, R^{2′} is CH₃. In some embodiments, R^{2′} is CH₂OH. In some embodiments, R^{2′} is CH₂OCH₃. In some embodiments, R^{2′} is CF₃.

In some embodiment in any of the aspects disclosed herein (e.g., any one of formulas (I), (II), (III), (IIIa), (IV), (IVa), (V), and (VI)), R^{2′} is optionally substituted C₆-C₁₄ aryl, e.g., phenyl.

In some embodiment in any of the aspects disclosed herein (e.g., any one of formulas (I), (II), (III), (IIIa), (IV), (IVa), (V), and (VI)), R^{2′} is optionally substituted C₁-C₆ alkoxy. In some embodiments, R^{2′} is OCH₃. In some embodiments, R² is OCHF₂. In some embodiments, R^{2′} is OCF₃.

In some embodiment in any of the aspects disclosed herein (e.g., any one of formulas (I), (II), (III), (IIIa), (IV), (IVa), (V), and (VI)), R^{2′} is optionally substituted C₆-C₁₄ aryloxy, e.g., phenoxy.

In some embodiment in any of the aspects disclosed herein (e.g., any one of formulas (I), (II), (III), (IIIa), (IV), (IVa), (V), and (VI)), R^{2′} is halo, e.g., F.

In some embodiment in any of the aspects disclosed herein (e.g., any one of formulas (I), (II), (III), (IIIa), (IV), (IVa), (V), and (VI)), R^{2′} is SO₂R^{a}. In some embodiments, R^{2′} is SO₂CH₃. In some embodiments, R^{2′} is SO₂CH₂CH₃. In some embodiments, R^{2′} is

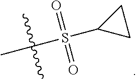 [SMILES: *S(=O)(=O)C1CC1]

In some embodiment in any of the aspects disclosed herein (e.g., any one of formulas (I), (II), (III), (IIIa), (IV), (IVa), (V), and (VI)), e.g., R^{2′} is S(O)(NH)R^{b}, e.g., S(O)(NH)CH₃.

In some embodiment in any of the aspects disclosed herein (e.g., any one of formulas (I), (II), (III), (IIIa), (IV), (IVa), (V), and (VI)), R^{2′} is optionally substituted 5- to 10-membered heterocycle, e.g.,

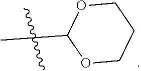 [SMILES: *C1OCCCO1]

In some embodiment in any of the aspects disclosed herein (e.g., any one of formulas (I), (II), (III), (IIIa), (IV), (IVa), (V), and (VI)), R^{2′} is optionally substituted 5- to 10-membered heteroaryl, e.g.,

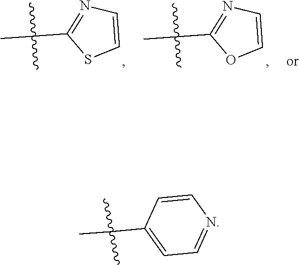 [SMILES: *c1nccs1 ; *c1ncco1 ; *c1ccncc1]

In some embodiment in any of the aspects disclosed herein (e.g., any one of formulas (I), (II), (III), (IIIa), (IV), (IVa), (V), and (VI)), R² and R^{2′} combine to form ═C(R^{c})₂, e.g., ═CH₂.

In some embodiment in any of the aspects disclosed herein (e.g., any one of formulas (I), (II), (III), (IIIa), (IV), (IVa), (V), and (VI)), R¹ is H.

In some embodiment in any of the aspects disclosed herein (e.g., any one of formulas (I), (II), (III), (IIIa), (IV), (IVa), (V), and (VI)), R^{1′} is H.

In some embodiment in any of the aspects disclosed herein (e.g., any one of formulas (I), (II), (III), (IIIa), (IV), (IVa), (V), and (VI)), Y is

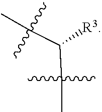

In some embodiment in any of the aspects disclosed herein (e.g., any one of formulas (I), (II), (III), (IIIa), (IV), (IVa), (V), and (VI)), R³ is H. In some embodiment in any of the aspects disclosed herein (e.g., any one of formulas (I), (II), (III), (IIIa), (IV), (IVa), (V), and (VI)), R³ is CH₃. In some embodiment in any of the aspects disclosed herein (e.g., any one of formulas (I), (II), (III), (IIIa), (IV), (IVa), (V), and (VI)), R³ is CF₃.

In some embodiment in any of the aspects disclosed herein (e.g., any one of formulas (I), (II), (III), (IIIa), (IV), (IVa), (V), and (VI)), Y is

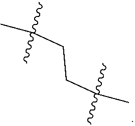 [SMILES: *CC*]

In some embodiment in any of the aspects disclosed herein (e.g., any one of formulas (I), (II), (III), (IIIa), (IV), (IVa), (V), and (VI)), X is N(R^{d})₂, e.g., NH₂. In some embodiment in any of the aspects disclosed herein (e.g., any one of formulas (I), (II), (III), (IIIa), (IV), (IVa), (V), and (VI)), X is NHOC(O)(C₁-C₆ alkyl), e.g., NHOC(O)CH₃. In some embodiment in any of the aspects disclosed herein (e.g., any one of formulas (I), (II), (III), (IIIa), (IV), (IVa), (V), and (VI)), X is NHOH.

In some embodiment in any of the aspects disclosed herein (e.g., any one of formulas (I), (II), (III), (IIIa), (IV), (IVa), (V), and (VI)), R⁴ is H. In some embodiment in any of the aspects disclosed herein (e.g., any one of formulas (I), (II), (III), (IIIa), (IV), (IVa), (V), and (VI)), R⁴ is C(O)OR^{e}. In some embodiments, R⁴ is C(O)OCH₃. In some embodiments, R⁴ is C(O)O(CH₂)₅CH₃. In some embodiments, R⁴ is C(O)OC₆H₅.

In some embodiment in any of the aspects disclosed herein (e.g., any one of formulas (I), (II), (III), (IIIa), (IV), (IVa), (V), or (VI)), X and R⁴, together with the atoms to which each is attached, form 1H-imidazole-2-yl. In some embodiment in any of the aspects disclosed herein (e.g., any one of formulas (I), (II), (III), (IIIa), (IV), (IVa), (V), and (VI)), X and R⁴, together with the atoms to which each is attached, form (5-aminothiazol-2-yl)thiopheny-2-yl.

In some embodiments in any of the aspects disclosed herein (e.g., any one of formulas (I), (II), (III), (IIIa), (IV), (IVa), (V), and (VI)), X⁵ is S, and X⁶ and X⁷ are both CH. In some embodiments in any of the aspects disclosed herein (e.g., any one of formulas (I), (II), (III), (IIIa), (IV), (IVa), (V), and (VI)) X⁶ is S, and X⁵ and X⁷ are both CH. In some embodiments in any of the aspects disclosed herein (e.g., any one of formulas (I), (II), (III), (IIIa), (IV), (IVa), (V), and (VI)) X⁷ is S, and X⁵ and X⁶ are both CH.

In some embodiment in any of the aspects disclosed herein (e.g., any one of formulas (I), (II), (III), (IIIa), (IV), (IVa), (V), and (VI)), A is H.

In another aspect, the present disclosure provides compounds compounds of Table 1 and pharmaceutically acceptable salts thereof.

In another aspect, the present disclosure provides a pharmaceutical composition including a compound disclosed herein (e.g., any one of the compounds of formulas (I), (II), (III), (IIIa), (IV), (IVa), (V), and (VI) and Table 1) or a pharmaceutically acceptable salt thereof a pharmaceutically acceptable excipient.

In another aspect, the present disclosure provides a method of treating a complement C1 esterase (C1s) mediated disorder, comprising administering to a subject in need thereof a therapeutically effective amount of a compound disclosed herein (e.g., any one of the compounds of formulas (I), (II), (III), (IIIa), (IV), (IVa), (V), and (VI) and Table 1) or a pharmaceutically acceptable salt thereof.

In some embodiments, the subject is a human.

In some embodiments, the disorder is acute antibody-mediated rejection, amyotrophic lateral sclerosis, autoimmune blistering disease, bullous pemphigoid, chronic inflammatory demyelinating polyneuropathy, geographic atrophy, Guillain-Barre Syndrome, Huntington's Disease, immune thrombocytopenia purpura, lupus nephritis, multifocal motor neuropathy, rheumatoid arthritis, traumatic brain injury, and warm autoimmune hemolytic anemia.

In another aspect, the present disclosure provides a compound disclosed herein (e.g., any one of the compounds of formulas (I), (II), (III), (IIIa), (IV), (IVa), (V), and (VI) and Table 1) or a pharmaceutically acceptable salt thereof, for use in the treatment of a C1s mediated disorder.

In another aspect, the present disclosure provides a use of a compound disclosed herein (e.g., any one of the compounds of formulas (I), (II), (III), (IIIa), (IV), (IVa), (V), and (VI) and Table 1) or a pharmaceutically acceptable salt thereof in the preparation a medicament for use in the treatment of a C1s mediated disorder.

### Definitions

Compounds are described using standard nomenclature. Unless defined otherwise, all technical and scientific terms used herein have the same meaning as is commonly understood by one of skill in the art to which the claimed invention belongs.

The compounds in any of the Formulas described herein include enantiomers, mixtures of enantiomers, diastereomers, tautomers, racemates and other isomers, such as rotamers, as if each is specifically described, unless otherwise indicated or otherwise excluded by context.

The terms “a” and “an” do not denote a limitation of quantity, but rather denote the presence of at least one of the referenced items. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely as illustration, and does not pose a limitation on the scope of the invention, which is defined by the claims.

The term “substituted”, as used herein, means that any one or more hydrogens on the designated atom or group is replaced with a moiety selected from the indicated group, provided that the designated atom's normal valence is not exceeded, and the resulting compound is stable. For example, when the substituent is oxo (i.e., ═O) then two hydrogens on the atom are replaced. For example, a pyridyl group substituted by oxo is a pyridone. Combinations of substituents and/or variables are permissible only if such combinations result in stable compounds or useful synthetic intermediates.

The term “alkoxy,” as used herein, refers to a —OR radical, in which R is alkyl, as defined herein.

The term “alkyl,” as used herein, refers to a branched or straight-chain monovalent saturated aliphatic radical containing only C and H when unsubstituted. The monovalency of an alkyl group does not include the optional substituents on the alkyl group. For example, if an alkyl group is attached to a compound, monovalency of the alkyl group refers to its attachment to the compound and does not include any additional substituents that may be present on the alkyl group. In some embodiments, the alkyl group may contain, e.g., 1-8, 1-6, 1-4, or 1-2 carbon atoms (e.g., C₁-C₃, C₁-C₆, C₁-C₄, or C₁-C₂). Examples include, but are not limited to, methyl, ethyl, isobutyl, sec-butyl, tert-butyl, 2-methylpropyl, and 2,2-dimethylpropyl.

The term “alkylene,” as used herein, refers to a divalent radical obtained by removing a hydrogen atom from a carbon atom of an alkyl group. The divalency of an alkylene group does not include the optional substituents on the alkylene group. Examples of alkylene groups include, but are not limited to, methylene, ethylene, and n-propylene.

The term “aryl,” as used herein, refers to any monocyclic or fused ring bicyclic or multicyclic system containing only carbon atoms in the ring(s), which has the characteristics of aromaticity in terms of electron distribution throughout entire the ring system, e.g., phenyl, naphthyl, or phenanthryl. An aryl group may have, e.g., six to sixteen carbons (e.g., six carbons, ten carbons, thirteen carbons, fourteen carbons, or sixteen carbons).

The term “arylene,” as used herein refers to a divalent radical obtained by removing a hydrogen atom from a carbon atom of an aryl group. The divalency of an arylene group does not include the optional substitutents on the arylene group. Phenylene is a non-limiting example of an arylene group.

The term “aryloxy,” as used herein, refers to an —OR radical, in which R is aryl, as defined herein.

The term “cycloalkyl,” as used herein, refers to a monovalent, saturated cyclic group containing only C and H when unsubstituted. A cycloalkyl may have, e.g., three to eight carbons (e.g., a C₃-C₄, C₃-C₅, C₃-C₆, C₃-C₇, or C₃-C₈ cycloalkyl). Examples of cycloalkyls include, but are not limited to, cyclopropyl, cyclobutyl, cyclopentyl, cyclohexyl, and cycloheptyl. The term “cycloalkyl” also includes cyclic groups having a bridged multicyclic structure in which one or more carbons bridges two non-adjacent members of a monocyclic ring, e.g., bicyclo[2.2.1]heptyl.

The term “halo,” as used herein, refers to a fluorine (fluoro), chlorine (chloro), bromine (bromo), or iodine (iodo) radical.

The term “heteroaryl,” as used herein, refers to a monocyclic, bicyclic, or multicyclic aromatic ring system having at least one ring that contains 1, 2, or 3 heteroatoms selected from N, O, S, B, and P (e.g., 1, 2, or 3 heteroatoms selected from N, O, and S) as ring atoms, with the remaining ring atoms being carbon. In some embodiments, a heteroaryl group is a bicyclic or tricyclic system containing at least one 5, 6, or 7 membered aromatic ring which contains from 1, 2, 3, or 4 heteroatoms selected from N, O, S, B or P (e.g., 1, 2, 3, or 4 heteroatoms selected from N, O, and S) as ring atoms, with the remaining ring atoms being carbon. In some embodiments, a heteroaryl group is a monocyclic aromatic ring having 5 or 6 ring atoms (i.e., 5- or 6-membered heteroaryl). In some embodiments, is a bicyclic aromatic ring system having 8 to 10 ring atoms (i.e., 8- to 10-membered bicyclic heteroaryl). Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl.

The term “heterocycle,” as used herein, refers to saturated or unsaturated, non-aromatic, monocycic, biciyclic, or multicyclic group containing 1, 2, 3, or 4 heteratoms as ring atoms, with the remaining ring atoms being carbon. The term “heterocycle” includes monocyclic 3- to 12-membered rings, as well as bicyclic 5- to 16-membered ring systems (which can include fused, bridged, or spiro, bicyclic ring systems). Examples of saturated heterocycles include saturated 4- to 7-membered monocyclic groups containing 1 to 4 nitrogen atoms (e.g., pyrrolidinyl, imidazolidinyl, piperidinyl, pyrrolinyl, azetidinyl, piperazinyl, and pyrazolidinyl); saturated 4 to 6-membered monocyclic groups containing 1 to 2 oxygen atoms and 1 to 3 nitrogen atoms (e.g., morpholinyl); saturated 3 to 6-membered monocyclic groups containing 1 to 2 sulfur atoms and 1 to 3 nitrogen atoms (e.g., thiazolidinyl). Examples of unsaturated, non-aromatic heterocycles radicals include but are not limited to, dihydrothienyl, dihydropyranyl, dihydrofuryl, and dihydrothiazolyl. Other examples heterocycles include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl. “Bicyclic heterocycle” includes groups in which a saturated or unsaturated, non-aromatic ring containin 1, 2, 3, or 4 heteroatoms as ring atoms is fused with an aryl group (e.g., phenyl) or a cycloalkyl group. “Bicyclic heterocycle” also includes groups in which a heteroaryl group, as defined herein, is fused to a saturated or unsaturated, non-aromatic ring containing 0, 1, 2, 3, or 4 heteroatoms as ring atoms.

The term “oxo,” as used herein, refers to a ═O radial.

The phrase “optionally substituted X,” as used herein, is intended to be equivalent to “X, in which X is optionally substituted” (e.g., “alkyl, in which said alkyl is optionally substituted”). It is not intended to mean that the feature “X” (e.g., alkyl) per se is optional. The term “optionally substituted,” as used herein, refers to having 0, 1, or more substituents (e.g., 0-10 substituents, 0-5 substituents, or 0, 1, 2, 3, 4, 5, 6, 7, 8, 9, or 10 substitutents).

Alkyl, alkylene, alkoxy, cycloalkyl, aryl, arylene, aryloxy, heteroaryl, and heterocycle groups may be substituted with cycloalkyl; aryl; heteroaryl; heterocycle; halo; OR^{a}, in which R^{a} is H, alkyl, cycloalkyl, aryl, heteroaryl, or heterocycle; SR^{a}, in which R^{a} is H, alkyl, cycloalkyl, aryl, heteroaryl, or heterocycle; CN; NO₂; N₃; NR^{b}R^{c}; in which each of R^{b} and R^{c} is, independently, H, alkyl, cycloalkyl, aryl, heteroaryl, or heterocycle; SO₂R^{d}, in which R^{d} is H, alkyl or aryl; SO₂NR^{e}R^{f}, in which each of R^{e} and R^{f} is, independently, H, alkyl, or aryl; SOR^{g}, in which R^{g} is H, alkyl, or aryl; or SiR^{h}Rⁱ, in which R^{h} and Rⁱ is, independently, H or alkyl. Aryl, cycloalkyl, heteroaryl, and heterocycle groups may also be substituted with alkyl. Alkyl, alkylene, cycloalkyl, and heterocycle groups may also be substituted with oxo or ═NR^{j}, in which R^{j} is H or alkyl. In some embodiments, a substituent is further substituted with one or more substituents as described herein. For example, a C₁ alkyl group, i.e., methyl, may be substituted with oxo to form a formyl group and further substituted with —OH or —NH₂ to form a carboxyl group or an amido group.

The term “pharmaceutical composition,” as used herein, refers to one or more active compounds, formulated together with one or more pharmaceutically acceptable excipients. In some embodiments, a compound of the disclosure (e.g., is present in a unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, or capsules; and parenteral administration, for example, by subcutaneous, intramuscular, or intravenous injection.

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid.

The term “pharmaceutically acceptable excipient,” as used herein, refers to any inactive ingredient (for example, a vehicle capable of suspending or dissolving the active compound) that is biocompatible and suitable for administration to a subject. Typical excipients include, for example: antiadherents, antioxidants, binders, coatings, compression aids, disintegrants, dyes, emollients, emulsifiers, diluents, film formers or coatings, flavors, fragrances, glidants, lubricants, preservatives, printing inks, sorbents, suspending or dispersing agents, sweeteners, or waters of hydration. Those of ordinary skill in the art are familiar with a variety of agents and materials useful as excipients.

The term “subject,” as used herein, can be a human, non-human primate, or other mammal, such as but not limited to dog, cat, horse, cow, pig, goat, monkey, rat, mouse, and sheep. In preferred embodiments, the subject is a human.

As used herein, and as well understood in the art, “to treat” a condition or “treatment” of various diseases and disorders is an approach for obtaining beneficial or desired results, such as clinical results. Beneficial or desired results can include, but are not limited to, alleviation of one or more symptoms or conditions; diminishment of extent of disease, disorder, or condition; stabilizing (i.e., not worsening) state of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

A “therapeutically effective amount” or an “effective amount” of an active compound pharmaceutical composition of the present disclosure refers an amount effective, when administered to a subject, to provide a therapeutic benefit, such as an amelioration of symptoms or reduction or dimunition of the disease itself. In one embodiment, a therapeutically effective amount is an amount sufficient to prevent a significant increase, or will significantly reduce, the detectable level of hemolysis in the patient's blood, serum, or tissues.

### DETAILS DESCRIPTION

### Active Compounds

The present disclosure provides compounds useful for the treatment of a disorder mediated by the complment cascade (e.g., a disorder mediated by C1s). In some embodiments, a compound of the present disclosure is described by formula (I):

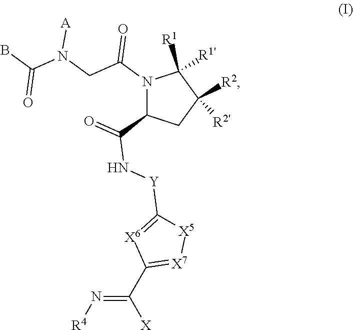

in which each of R¹, R^{1′}, R², and R^{2′} is independently selected from H; halo; optionally substituted C₁-C₆ alkyl; optionally substituted C₃-C₈ cycloalkyl; optionally substituted C₆-C₁₄ aryl; optionally substituted 5- to 10-membered heterocycle; optionally substituted 5- to 10-membered heteroaryl; optionally substituted C₁-C₆ alkoxy; optionally substituted C₆-C₁₄ aryloxy; SO₂R^{a}, in which R^{a} is H, C₁-C₆ alkyl, or C₃-C₈ cycloalkyl; and S(O)(NH)R^{b}, in which R^{b} is H or C₁-C₆ alkyl; in which at least one of R¹, R^{1′}, R², and R^{2′} is not H; or R¹ and R², together with the atoms to which each is attached, form optionally substituted C₃-C₈ cycloalkyl; or R² and R^{2′}, together with the atom to which they are attached, form optionally substituted 5- or 6-membered spirocyclic heterocycle; or R² and R^{2′} combine to form ═C(R^{c})₂, in which each R^{c} is independently H or halo; Y is selected from

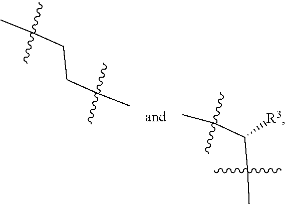

in which R³ is H, CH₃, or CF₃; X is N(R^{d})₂, in which each R^{d} is independently H; OH, OC(O)(C₁-C₆ alkyl); and R⁴ is H or C(O)OR^{e}, in which R^{e} is C₁-C₆ alkyl or C₆-C₁₄ aryl; or X and R⁴, together with the atoms to which each is attached, form 1H-imidazole-2-yl or (5-aminothiazol-2-yl)thiopheny-2-yl; A is H or C₁-C₆ alkyl; B is selected from:

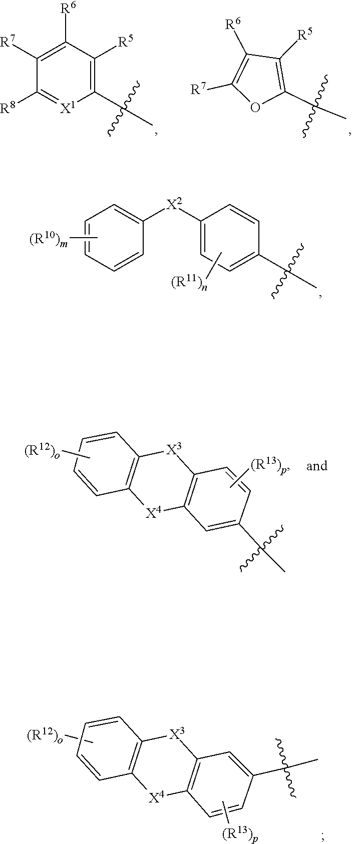

X¹ is CR⁹ or N; each of R⁵, R⁶, and R⁹ is independently selected from H, halo, and optionally substituted C₁-C₆ alkyl, or R⁵ and A combine to form optionally substituted C₁-C₂ alkylene; one of R⁷ and R⁸ is optionally substituted C₆-C₁₄ aryl or optionally substituted 5- to 10-membered heteroaryl; and the other is H, halo, or optionally substituted C₁-C₆ alkyl; or R⁷ and R⁸, together with the atoms to which each is attached; form optionally substituted 5- or 6-membered heterocycle, optionally substituted 5- to 10-membered heteroaryl, or optionally substituted C₆-C₁₄ aryl; or R⁶ and R⁹ combine to form (C₂-C₆alkylene)(C₆-C₁₄arylene)(C₂-C₆alkylene), and each of R₅, R₇, and R₈ is H; X² is O or C(R^{f})₂, in which each R^{f} is halo; m is selected from 0, 1, 2, 3, 4, and 5; n is selected from 0, 1, 2, 3, and 4; each R¹⁰ and R¹¹ is independently halo, CN, optionally substituted C₁-C₆ alkyl, optionally substituted C₁-C₆ alkoxy, or optionally substituted C₃-C₈ cycloalkyl; each of X³ and X⁴ is a bond; O; S; C(R^{g})₂, in which each R^{g} is independently H, OH, halo, optionally substituted C₁-C₆ alkyl, or optionally substituted C₁-C₆ alkoxy, or both R^{g} combine to form oxo; NR^{h}, in which R^{h} is H or C₁-C₆ alkyl; or SO₂; X⁵ is S, and X⁶ and X⁷ are both CH; or X⁶ is S, and X⁵ and X⁷ are both CH; or X⁷ is S, and X⁵ and X⁶ are both CH; o is selected from 0, 1, 2, 3, and 4; p is selected from 0, 1, 2, and 3; and each R¹² and R¹³ is independently halo, optionally substituted C₁-C₆ alkyl, optionally substituted C₁-C₆ alkoxy, or optionally substituted C₃-C₈ cycloalkyl, provided thatwhen B is

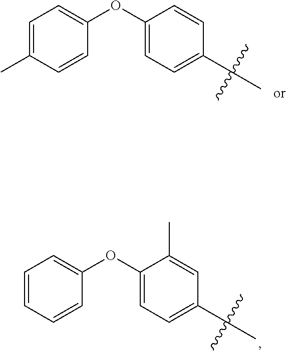 [SMILES: *c1ccc(Oc2ccc(C)cc2)cc1 ; *c1ccc(Oc2ccccc2)c(C)c1]

R³ is CH₃, CF₃, or CH₂OH; when m is 0, n is 1, 2, 3, or 4; and when n is 0, m is 1, 2, 3, 4, or 5.

In some embodiments, a compound of the present disclosure is described by any one of formulas (II), (III), (IIIa), (IV), (IVa), and (V).

In some embodiments, a compound of the present disclosure is a compound of Table 1.

### Pharmaceutical Compositions

A pharmaceutical composition of the invention contains one or more of the compounds disclosed herein (e.g., one or more of the compounds of formula (I), (II), (III), (IIIa), (IV), (IVa), (V), and (VI), and Table 1) as the therapeutic compound. In addition to a therapeutically effective amount of the compound, the pharmaceutical compositions also contain a pharmaceutically acceptable excipient, which can be formulated by methods known to those skilled in the art. In some embodiments, the pharmaceutical compositions for treating cancer contain one or more of the compounds disclosed herein (e.g., one or more of the compounds of formula (I), (II), (III), (IIIa), (IV), (IVa), (V), and (VI), and Table 1) may be formulated and/or administered with or without other therapeutics for a particular condition. Examples of such therapeutics (second therapeutic agents) are described herein.

The compounds disclosed herein (e.g., the compounds of formulas (I), (II), (III), (IIIa), (IV), (IVa), (V), and (VI), and Table 1) may be used in the form of free base, or in the form of salts, and as solvates. All forms are within the scope of the disclosure.

Exemplary routes of administration of the pharmaceutical compositions (or the compounds of the composition) include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration.

### Formulations for Oral Administration

The pharmaceutical compositions of the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Pharmaceutical compositions for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Formulations for Parenteral Administration

The pharmaceutical compositions of the invention can be administered in a pharmaceutically acceptable parenteral (e.g., intravenous, intramuscular, subcutaneous or the like) formulation as described herein. The pharmaceutical composition may also be administered parenterally in dosage forms or formulations containing conventional, non-toxic pharmaceutically acceptable carriers and adjuvants. In particular, formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water; water adjusted to a suitable pH by addition of an appropriate amount of hydrochloric acid, sodium hydroxide, or a suitable buffer; 1,3-butanediol; Ringer's solution; and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference in its entirety.

The parenteral formulation can be any of the five general types of preparations identified by the USP-NF as suitable for parenteral administration:
(1) “Drug Injection:” a liquid preparation that is a drug substance (e.g., a compound of the invention), or a solution thereof;
(2) “Drug for Injection:” the drug substance (e.g., a compound of the invention) as a dry solid that will be combined with the appropriate sterile vehicle for parenteral administration as a drug injection;
(3) “Drug Injectable Emulsion:” a liquid preparation of the drug substance (e.g., a compound of the invention) that is dissolved or dispersed in a suitable emulsion medium;
(4) “Drug Injectable Suspension:” a liquid preparation of the drug substance (e.g., a compound of the invention) suspended in a suitable liquid medium; and
(5) “Drug for Injectable Suspension:” the drug substance (e.g., a compound of the invention) as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropyl cellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 23^{rd} Ed., Adejare, Ed., Academic Press (2020) and in The United States Pharmacopeia and National Formulary (USP-NF 2021 Issues 1-3), published in 2021.

Formulations for parenteral administration may, for example, contain sterile water, saline, polyalkylene glycols (e.g., polyethylene glycol), oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain, for example, lactose, or may be aqueous solutions containing, for example, polyoxyethylene-9-lauryl ether, glycocholate and deoxycholate, or may be oily solutions for administration in the form of nasal drops, or as a gel.

### Uses of Active Compounds for Treatment of Selected Disorders

In one aspect, an effective amount of an active compound described herein (e.g., any one of the compounds of formulas (I), (II), (III), (IIIa), (IV), (IVa), (V), and (VI) and Table 1, or a pharmaceutically acceptable salt thereof) is used to treat a medical disorder which is an inflammatory or immune condition, a disorder mediated by the complement cascade (including a dysfunctional cascade) including a complement-related disorder or alternative complement pathway-related disorder, a disorder or abnormality of a cell that adversely affects the ability of the cell to engage in or respond to normal complement activity, or an undesired complement-mediated response to a medical treatment, such as surgery or other medical procedure or a pharmaceutical or biopharmaceutical drug administration, a blood transfusion, or other allogenic tissue or fluid administration.

A complement-mediated disease or disorder is a disease or disorder in which the amount or activity of complement is such as to cause disease or disorder in an individual.

In some embodiments, the complement-mediated disease or disorder is selected from the group consisting of autoimmune disease, cancer, hematological disease, infectious disease, inflammatory disease, ischemia-reperfusion injury, neurodegenerative disease, neurodegenerative disorder, ocular disease, renal disease, transplant rejection, vascular disease, and vasculitis disease.

In some embodiments, the complement-mediated disease or disorder is an autoimmune disease. In some embodiments, the complement-mediated disease or disorder is cancer.

In some embodiments, the complement-mediated disease or disorder is an infectious disease.

In some embodiments, the complement-mediated disease or disorder is an inflammatory disease.

In some embodiments, the complement-mediated disease or disorder is a hematological disease.

In some embodiments, the complement-mediated disease or disorder is an ischemia-reperfusion injury.

In some embodiments, the complement-mediated disease or disorder is ocular disease. In some embodiments, the complement-mediated disease or disorder is a renal disease.

In some embodiments, the complement-mediated disease or disorder is transplant rejection.

In some embodiments, the complement-mediated disease or disorder is antibody-mediated transplant rejection, e.g., acute antibody-mediated rejection.

In some embodiments, the complement-mediated disease or disorder is a vascular disease.

In some embodiments, the complement-mediated disease or disorder is a vasculitis disorder.

In some embodiments, the complement-mediated disease or disorder is a neurodegenerative disease or disorder.

In some embodiments, the complement-mediated disease is a neurodegenerative disease.

In some embodiments, the complement-mediated disorder is a neurodegenerative disorder. In some embodiments, the complement-mediated disease or disorder is a tauopathy.

In certain aspects, an effective amount of an active compound described herein, or it pharmaceutically acceptable salt, is used to treat a medical disorder of the central nervous system (CNS) or peripheral nervous system disorders involving complement activation. In some embodiments, the CNS disorder is an acquired brain or spinal cord injury, including, but not limited to ischaemic-reperfusion injury or stroke, traumatic brain injury (TBI) and spinal cord injury (SCI). In some embodiments, the CNS disorder is traumatic brain injury (TBI).

In some embodiments, the disorder is a neurodegeneration disorder. In embodiments, the disorder is a neuroinflammation disorder.

In certain aspects, an active compound described herein, or a pharmaceutically acceptable salt thereof, is used to treat Alzheimer's disease (AD). AD is characterized by two hallmark pathologies; amyloid-β (Aβ) plaques and neurofibrillary tangles comprising hyperphosphorylated tau. Recent studies have implicated complement in AD pathogenesis, including genome-wide association studies identifying single nucleotide polymorphisms (SNPs) associated with risk of late-onset AD in genes encoding complement proteins Clusterin (CLU) and CR1 (CR1). See Carpanini et al., Therapeutic Inhibition of the Complement System in Diseases of the Central Nervous System, Front. Immunol., 4 Mar. 2019. Biomarker studies have also identified complement proteins and activation products in plasma and/or CSF that distinguish AD from controls and predict risk of progression to AD. (Id.)

In certain aspects, an effective amount of active compound described herein, or a pharmaceutically acceptable salt thereof, is used to treat certain forms of frontotemporal dementia including, but not limited to, Pick's disease, sporadic Frontotemporal dementia and Frontotemporal dementia with Parkinsonism linked to chromosome 17, Progressive supranuclear palsy (PSP), Corticobasal degeneration (CBD), and Subacute sclerosing panencephalitis.

In certain aspects, an active compound described herein, or a pharmaceutically acceptable salt thereof, is used to treat Hungtington's disease.

In certain aspects, an effective amount of active compound described herein, or a pharmaceutically acceptable salt thereof, is used to treat multiple sclerosis (MS). Multiple sclerosis (MS) is the most common cause of neurological disability in young adults in northern European-Caucasian populations, with an approximate lifetime risk of one in 400. C3 has been shown to be deposited in the brains of MS patients. T-cell clone (TCC) has been shown to be in association with capillary endothelial cells, predominantly within plaques and adjacent white matter. Localization of C activation to areas of active myelin destruction has also been shown, with TCC deposited exclusively in such areas. C3d has been shown to be deposited in association with short segments of disrupted myelin in plaques with low-grade active demyelination and provides evidence for a C contribution to disease progression as well as acute inflammation. See Ingram et al., Complement in multiple sclerosis: its role in disease and potential as a biomarker. Clin Exp Immunol. 2009 February; 155(2):128-39.

In certain aspects, an effective amount of an active compound described herein, or a pharmaceutically acceptable salt thereof, is used to treat neuromyelitis optica (NMO). Neuromyelitis optica (NMO) is an inflammatory demyelinating disease affecting predominantly the optic nerves and spinal cord. Traditionally seen as a variant of MS, it has been redefined recently according to new criteria using a combination of phenotypic subtyping along with a newly developed biomarker of disease, NMO-immunoglobulin G (IgG) (reported sensitivity of 58-76% and specificity of 85-99% for NMO). NMO patients have higher levels of C3a and anti-C1q antibodies than healthy controls. C3a levels correlated with disease activity, neurological disability and aquaporin-4 IgG. Nytrova et al., Complement activation in patients with neuromyelitis optica. J Neuroimmunol. 2014 Sep. 15; 274(1-2):185-91.

In certain aspects, an effective amount of an active compound as described herein, or a pharmaceutically acceptable salt thereof, is used to treat amyotrophic lateral sclerosis (ALS). ALS is caused by progressive loss of upper and lower (a) motor neurons resulting in denervation of neuromuscular junctions in the peripheral nervous system, progressive muscle weakness, atrophy, spasticity, respiratory failure, and ultimately paralysis and death. Recent studies have shown increased C1q protein in motor cortex and spinal cord of ALS post-mortem tissue; C3 activation fragments and TCC in areas of pathology; C4d and TCC staining of degenerating neurons and glia in ALS motor cortex and spinal cord, and C5aR1 upregulation in areas of pathology. C3d and C4d have been found on oligodendroglia and degenerating neurites, surrounded by CR4-positive microglia, in spinal cord and motor cortex, and C1q, C3, and TCC have been shown to be present on motor end-plates in intercostal muscles in ALS donors even early in the disease process. See Carpanini et al., Therapeutic Inhibition of the Complement System in Diseases of the Central Nervous System, Front. Immunol., 4 Mar. 2019.

In certain aspects, an effective amount of an active compound described herein, or a pharmaceutically acceptable salt thereof, is used to treat Parkinson's disease (PD). PD is characterized by loss of dopaminergic neurons in the substantia nigra and deposits of the protein α-synuclein that form the pathological hallmarks of the disease, Lewy bodies. Patients present with resting tremor, bradykinesia, and rigidity. Complement activation has been associated with α-synuclein and Lewy bodies in Parkinson's disease; in vitro studies have demonstrated that the disease-associated splice variant α-synuclein 112, but not the full-length protein, cause activation of complement. In vivo, C3d, C4d, C7 and C9 localization in Lewy bodies has been reported. More recently, deposition of iC3b and C9 in Lewy bodies and melanized neurons has been reported, and iC3b immunoreactivity has been shown to be increased with normal ageing and was further elevated in PD vs. age-matched controls. Furthermore, correlation between the ratios of C3/Aβ42 or FH/Aβ42 in CSF and severity of Parkinson's disease motor and cognitive symptoms has been shown. See Carpanini et al., Therapeutic Inhibition of the Complement System in Diseases of the Central Nervous System, Front. Immunol., 4 Mar. 2019. In some embodiments, the subject to be treated suffers from Parkinson's Disease with dementia (PDD).

In certain aspects, an effective amount of an active compound described herein, or a pharmaceutically acceptable salt thereof, is used to treat Huntington's disease (HD). HD is an autosomal dominant, inherited neurodegenerative disease characterized by progressive motor symptoms, psychiatric disturbances, and dementia. It is caused by expansion of a three-base-pair (CAG) repeat (39-121 repeats vs. normal range 8-39 repeats) in exon 1 of the HTT gene that translates into a polyglutamine tract at the N-terminus of the protein. This results in a polyglutamine length-dependent misfolding and accumulation of huntingtin protein in the striatum and cortex (layers 3, 5, and 6) followed by neuronal loss in these areas which spreads to the hippocampus. It has been shown that neurons, astrocytes, and myelin sheaths in the HD caudate and striatum were immunoreactive for C1q, C4, C3 and neo-epitopes in iC3b and TCC. Expression of mRNA encoding early complement components C1q (c-chain), C1r, C3, and C4, complement regulators C1NH, Clusterin, MCP, DAF and CD59, and complement receptors C3a and C5a, have been shown to be upregulated in the HD striatum, see Carpanini et al., Therapeutic Inhibition of the Complement System in Diseases of the Central Nervous System, Front. Immunol., 4 Mar. 2019.

In certain aspects, an effective amount of an active compound described herein, or a pharmaceutically acceptable salt thereof, is used to treat argyrophilic grain dementia, British type amyloid angiopathy, cerebral amyloid angiopathy, Creutzfeldt-Jakob disease, dementia pugilistica, diffuse neurofibrillary tangles with calcification, Down's syndrome, frontotemporal lobar degeneration, Gerstmann-Straussler-Scheinker disease, Hallervorden-Spatz disease, inclusion body myositis, multiple system atrophy (MSA), myotonic dystrophy, Niemann-Pick disease type C, non-Guamanian motor neuron disease with neurofibrillary tangles, postencephalitic parkinsonism, prion protein cerebral amyloid angiopathy, progressive subcortical gliosis, progressive supranuclear palsy, subacute sclerosing panencephalitis, Tangle only dementia, multi-infarct dementia, ischemic stroke, chronic traumatic encephalopathy (CTE), traumatic brain injury (TBI), and stroke.

In certain aspects, an effective amount of an active compound described herein, or a pharmaceutically acceptable salt thereof, is used to treat a hereditary motor and sensory neuropathy (HMSN).

In some embodiments, the hereditary and sensory neuropathy is Charcot-Marie-Tooth (CMT) disease.

In some embodiments, the HSMN is Charcot-Marie-Tooth disease type 1A or type 1B.

In some embodiments, the HSMN is Charcot-Marie-Tooth disease type 2.

In some embodiments, the HSMN is Dejerine-Sottas disease (Charcot-Marie-Tooth type 3).

In some embodiments, the HSMN is Refsum disease.

In some embodiments, the HSMN is Charcot-Marie-Tooth with pyramidal features. In some embodiments, the HSMN is Charcot-Marie-Tooth type 6. In some embodiments, the HSMN is HMSN+retinitis pigmentosa.

In some embodiments, an effective amount of an active compound described herein, or a pharmaceutically acceptable salt thereof, is used to treat Churg-Strauss syndrome.

In some embodiments, an effective amount of an active compound described herein, or a pharmaceutically acceptable salt thereof, is used to treat a peripheral artery disease (PAD).

In certain aspects, an effective amount of an active compound described herein, or a pharmaceutically acceptable salt thereof, is used to treat myasthenia gravis with CNS involvement.

In certain aspects, an effective amount of an active compound described herein, or a pharmaceutically acceptable salt thereof, is used to treat dementia with Lewy bodies.

In certain aspects, an effective amount of an active compound described herein, or a pharmaceutically acceptable salt thereof, is used to treat an individual suffering from prion disease.

In certain aspects, an effective amount of an active compound described herein, or a pharmaceutically acceptable salt thereof, is used to treat Behcet's Disease.

In certain aspects, an effective amount of an active compound described herein, or a pharmaceutically acceptable salt thereof, is used to treat congenital myasthenia.

In certain aspects, an effective amount of an active compound described herein, or a pharmaceutically acceptable salt thereof, is used to treat subacute sclerosing panencephalitis (SSPE).

In certain aspects, an effective amount of an active compound described herein, or a pharmaceutically acceptable salt thereof, is used to treat Guillain-Barré syndrome.

In certain aspects, the CNS disorder to be treated is a demyelinating disease, including, but not limited to, demyelinating myelinoclastic diseases and demyelinating leukostrophic disease.

In certain aspects, the disorder to be treated is a demyelinating myelonoclastic disease including, but not limited to, multiple sclerosis, neuromyelitis optica, neuromyelitis optica spectrum of disorders (NMOSD), idiopathic inflammatory demyelinating diseases (IIDD), anti-NMDA receptor encephalitis, acute disseminated encephalomyelitis, anti-MOG autoimmune encephalomyelitis, chronic relapsing inflammatory optic neuritis (CRION), acute disseminated encephalomyelitis (ADEM), immune-mediated encephalomyelitis, progressive multifocal leukoencephalopathy (PML); McDonalds-positive multiple sclerosis, acute hemorrhagic leukoencephalitis, Rasmussen's Encephalitis, Marburg multiple sclerosis, pseudotumefactive and tumefactive multiple sclerosis, Balo concentric sclerosis, diffuse myelinoclastic sclerosis, solitary sclerosis, multiple sclerosis with cavitary lesions, myelocortical multiple sclerosis (MCMS), atypical optic-spinal multiple sclerosis, pure spinal multiple sclerosis, HLA DRB3*02:02 multiple sclerosis, autoimmune GFAP astrocytopathy, Chronic inflammatory demyelinating polyneuropathy (CIDP), Guillain-Barré syndrome, progressive inflammatory neuropathy, Lewis-Sumner Syndrome, combined central and peripheral demyelination (CCPD), Bickerstaff brainstem encephalitis, Fisher syndrome, trigeminal neuralgia, NMDAR anti-NMDA receptor encephalitis, primary progressive MS (PPMS), OPA1 variant multiple sclerosis, KIR4.1 multiple sclerosis, aquaporine-related multiple sclerosis, chronic cerebrospinal venous insufficiency (CCSVI or CCVI), diffuse sclerosis, and Schilder's disease.

In certain aspects, the disorder to be treated is a demyelinating leukostrophic disease including, but not limited to, myelitis, central pontine myelinolysis (CPM), extrapontine myelinolysis, tabes dorsalis, progressive multifocal leukoencephalopathy, leukoencephalopathy with vanishing white matter, leukoencephalopathy with neuroaxonal spheroids, reversible posterior leukoencephalopathy syndrome, megalencephalic leukoencephalopathy with subcortical cysts, megalencephalic leukoencephalopathy with subcortical cysts 1, hypertensive leukoencephalopathy, Metachromatic leukodystrophy, Krabbe disease, Canavan disease, X-linked adrenoleukodystrophy, Alexander disease, cerebrotendineous xanthomatosis, Pelizaeus-Merzbacher disease, and Refsum disease.

In some embodiments, an effective amount of an active compound described herein, or a pharmaceutically acceptable salt thereof, is used to treat Buerger's disease, also known as thromboangiitis obliterans.

In some embodiments, an effective amount of an active compound described herein, or a pharmaceutically acceptable salt thereof, is used to treat giant cell arteritis.

In some embodiments, an effective amount of an active compound described herein, or a pharmaceutically acceptable salt thereof, is used to treat Raynaud's disease.

In certain aspects, the disorder to be treated is a demyelinating disease of the peripheral nervous system, including, but not limited to, Guillain-Barré syndrome and its chronic counterpart, chronic inflammatory demyelinating polyneuropathy, anti-MAG peripheral neuropathy, Charcot-Marie-Tooth disease and its counterpart Hereditary neuropathy with liability to pressure palsy, Copper deficiency-associated conditions (peripheral neuropathy, myelopathy, and rarely optic neuropathy), and progressive inflammatory neuropathy.

In certain aspects, the disorder to be treated is a neurological inflammatory disorder. In certain embodiments, the disorder to be treated includes, but is not limited to, cranial arteritis; giant cell arteritis; Holmes-Adie syndrome; inclusion body myositis (IBM); meningitis; neurologic paraneoplastic syndrome including, but not limited to, Lambert-Eaton myasthenic syndrome, stiff-person syndrome, encephalomyelitis (inflammation of the brain and spinal cord), myasthenia gravis, cerebellar degeneration, limbic and/or brainstem encephalitis, neuromyotonia, and opsoclonus (involving eye movement) and sensory neuropathy; polymyositis; transverse myelitis; vasculitis including temporal arteritis; arachnoiditis; Kinsbourne syndrome or opsoclonus myoclonus syndrome (OMS); or Saint Vitus Dance or sydenham chorea (SD) disease.

In some embodiments, an effective amount of an active compound described herein, or a pharmaceutically acceptable salt thereof, is used to treat transverse myelitis.

In certain aspects, the disorder to be treated is a peripheral neuropathy. In some embodiments, the peripheral neuropathy is a mononeuropathy. In some embodiments, the neuropathy is a polyneuropathy. In some embodiments, the polyneuropathy is distal axonopathy, diabetic neuropathy, a demyelinating polyneuropathy, small fiber peripheral neuropathy, mononeuritis multiplex, polyneuritis multiplex, autonomic neuropathy, or neuritis.

In some embodiments, an effective amount of an active compound described herein, or a pharmaceutically acceptable salt thereof, is used to treat multifocal motor neuropathy.

In some embodiments, an effective amount of an active compound described herein, or a pharmaceutically acceptable salt thereof, is used to treat an autoimmune vascular disease. In some embodiments, the autoimmune vascular disease is vasculitis. In some embodiments, the vasculitis includes, but is not limited to, autoimmune inflammatory vasculitis, Cutaneous small-vessel vasculitis, Granulomatosis with polyangiitis, Eosinophilic granulomatosis with polyangiitis, Behçet's disease, Kawasaki disease, Buerger's disease, and “Limited” granulomatosis with polyangiitis vasculitis.

In some embodiments, an active compound or its salt or composition as described herein is used to treat an arteritis. Is some embodiments, the arteritis includes, but is not limited to, giant cell arteritis, Takayasu arteritis, temporal arteritis, and polyarteritis nodosa.

In some embodiments, a method for the treatment of a glomerulonephritis is provided. In some embodiment, the glomerulonephritis is membranoproliferative glomerulonephritis (MPGN). In some embodiments, the MPGN is MPGN Type I. In some embodiments, the MPGN is MPGN Type II. In some embodiments, the MPGN is MPGN Type III. In some embodiments, the MPGN is C3 glomerulonephritis (C3G). In some embodiments, the MPGN is dense deposit disease (DDD). In some embodiments, the MPGN is a C4 deposition disorder.

In some embodiments, the glomerulonephritis is IC-MPGN. In some embodiments, the glomerulonephritis is a membraneous glomerulonephritis. In some embodiments, the glomerulonephritis is IgA nephropathy. In some embodiments, the glomerulonephritis is post-infectious glomerulonephritis. In some embodiments, the glomerulonephritis is a rapidly progressive glomerulonephritis, for example Type I (Goodpasture syndrome), Type II, or Type III rapidly progressive glomerulonephritis.

In some embodiments, a method for the treatment of paroxysmal nocturnal hemoglobinuria (PNH) is provided that includes the administration of an effective amount of a compound disclosed herein (e.g., any one of the compounds of formulas (I), (II), (III), (IIIa), (IV), (IVa), (V), and (VI) and Table 1) or a pharmaceutically acceptable salt thereof to a subject, optionally in a pharmaceutically acceptable composition.

In some embodiments, a method for the treatment of hereditary angioedema (HAE) is provided that includes the administration of an effective amount of a compound disclosed herein (e.g., any one of the compounds of formulas (I), (II), (III), (IIIa), (IV), (IVa), (V), and (VI) and Table 1) or a pharmaceutically acceptable salt thereof to a subject, optionally in a pharmaceutically acceptable composition. Mutations in the SERPING1 gene cause hereditary angioedema type I and type II. Hereditary angioedema is a disorder characterized by recurrent episodes of severe swelling (angioedema). The most common areas of the body to develop swelling are the limbs, face, intestinal tract, and airway. The SERPING1 gene provides instructions for making the C1 inhibitor protein, which is important for controlling inflammation. C1 inhibitor blocks the activity of certain proteins that promote inflammation. Mutations that cause hereditary angioedema type I lead to reduced levels of C1 inhibitor in the blood, while mutations that cause type II result in the production of a C1 inhibitor that functions abnormally. Without the proper levels of functional C1 inhibitor, excessive amounts of a protein fragment (peptide) called bradykinin are generated. Bradykinin promotes inflammation by increasing the leakage of fluid through the walls of blood vessels into body tissues. Excessive accumulation of fluids in body tissues causes the episodes of swelling seen in individuals with hereditary angioedema type I and type II.

In some embodiments, a method for the treatment of cold agglutinin disease (CAD) is provided that includes the administration of an effective amount of a compound disclosed herein (e.g., any one of the compounds of formulas (I), (II), (III), (IIIa), (IV), (IVa), (V), and (VI) and Table 1) or a pharmaceutically acceptable salt thereof to a subject, optionally in a pharmaceutically acceptable composition. CAD is a rare autoimmune hemolytic condition with potentially serious acute and chronic consequences that are driven by C1 activation of the classical complement pathway.

In some embodiments, a method for the treatment of atypical hemolytic uremic syndrome (aHUS) is provided that includes the administration of an effective amount of a compound disclosed herein (e.g., any one of the compounds of formulas (I), (II), (III), (IIIa), (IV), (IVa), (V), and (VI) and Table 1) or a pharmaceutically acceptable salt thereof to a subject, optionally in a pharmaceutically acceptable composition. Atypical hemolytic-uremic syndrome is a disease that primarily affects kidney function. Atypical hemolytic uremic syndrome, which can occur at any age, causes abnormal blood clots (thrombi) to form in small blood vessels in the kidneys. These clots can cause serious medical problems if they restrict or block blood flow. Atypical hemolytic-uremic syndrome is characterized by three major features related to abnormal clotting: hemolytic anemia, thrombocytopenia, and kidney failure.

In another embodiment, a method for the treatment of wet or dry age-related macular degeneration (AMD) in a subject is provided that includes the administration of an effective amount of a compound disclosed herein (e.g., any one of the compounds of formulas (I), (II), (III), (IIIa), (IV), (IVa), (V), and (VI) and Table 1) or a pharmaceutically acceptable salt thereof to a subject, optionally in a pharmaceutically acceptable composition. In another embodiment, a method for the treatment of rheumatoid arthritis in a subject is provided that includes the administration of an effective amount of a compound disclosed herein (e.g., any one of the compounds of formulas (I), (II), (III), (IIIa), (IV), (IVa), (V), and (VI) and Table 1) or a pharmaceutically acceptable salt thereof to a subject, optionally in a pharmaceutically acceptable composition.

In another embodiment, a method for the treatment of multiple sclerosis in a subject is provided that includes the administration of an effective amount of a compound disclosed herein (e.g., any one of the compounds of formulas (I), (II), (III), (IIIa), (IV), (IVa), (V), and (VI) and Table 1) or a pharmaceutically acceptable salt thereof to a subject, optionally in a pharmaceutically acceptable composition.

The active compounds or pharmaceutically acceptable salts thereof disclosed herein, are also useful for administration in combination (in the same or a different dosage form) or alternation with a second pharmaceutical agent for use in ameliorating or reducing a side effect of the second pharmaceutical agent.

For example, in some embodiments, the active compound may be used in combination with an adoptive cell-transfer therapy to reduce an inflammatory response associated with such therapy, for example, a cytokine mediated response such as cytokine response syndrome.

In some embodiments, the adoptive cell-transfer therapy is a chimeric antigen receptor T-Cell (CAR T) or a dendritic cell used to treat a hematologic or solid tumor, for example, a B-cell related hematologic cancer.

In some embodiments, the hematologic or solid tumor is acute lymphoblastic leukemia (ALL), acute myeloid leukemia (AML), non-Hodgkin's lymphoma, chronic lymphocytic leukemia (CLL), pancreatic cancer, glioblastoma, or a cancer that expresses CD19.

In some embodiments, the adoptive cell-transfer therapy is a non-engineered T-cell therapy, in which the T-cells have been activated and/or expanded to one or more viral or tumor antigens. In some embodiments, the associated inflammatory response is a cytokine mediated response.

In some embodiments, the second pharmaceutical agent is a cell that has been transformed to express a protein, in which the protein in the subject is mutated or otherwise has impaired function. In some embodiments, the transformed cell includes a CRISPR gene.

Another embodiment is provided that includes the administration of an effective amount of an active compound or a pharmaceutically acceptable salt thereof, optionally in a pharmaceutically acceptable composition to a subject to treat an ocular, pulmonary, gastrointestinal, or other disorder.

Any of the compounds described herein (e.g. a compound disclosed herein (e.g., any one of the compounds of formulas (I), (II), (III), (IIIa), (IV), (IVa), (V), and (VI) and Table 1) or a pharmaceutically acceptable salt thereof to a subject can be administered to the eye in any desired form of administration, including via intravitreal, intrastromal, intracameral, sub-tenon, sub-retinal, retro-bulbar, peribulbar, suprachorodial, choroidal, subchoroidal, conjunctival, subconjunctival, episcleral, posterior juxtascleral, scleral, circumcorneal, and tear duct injections, or through a mucus, mucin, or a mucosal barrier, in an immediate or controlled release fashion. In certain embodiments, the active compound includes a lipophilic group, such as a lipophilic acyl group, which is delivered to the eye in a polymeric drug delivery system such as polylactic acid, polylactide-co-glycolide, polyglycolide or other erodible polymer, or a combination thereof, or in another type of lipophilic material for ocular delivery. In some embodiments, the lipophilic active molecule is more soluble in the polymeric or other form of delivery system than in ocular fluid.

In other embodiments of the disclosure, an active compound provided herein can be used to treat or prevent a disorder in a subject mediated by complement. As examples, the disclosure includes methods to treat or prevent complement associated disorders that are induced by antibody-antigen interactions, a component of an immune or autoimmune disorder or by ischemic injury. The disclosure also provides methods to decrease inflammation or an immune response, including an autoimmune response, where mediated or affected by the classical complement pathway.

In some embodiments, the disorder is selected from fatty liver and conditions stemming from fatty liver, such as nonalcoholic steatohepatitis (NASH), liver inflammation, cirrhosis, and liver failure. In some embodiments of the present disclosure, a method is provided for treating fatty liver disease in a subject by administering an effective amount of an active compound or its salt or composition as described herein.

In another embodiment, an active compound or its salt or composition as described herein is used to modulate an immune response prior to or during surgery or other medical procedure. One non-limiting example is use in connection with acute or chronic graft versus subject disease, which is a common complication as a result of organ transplantation, allogeneic tissue transplant, and can also occur as a result of a blood transfusion.

In some embodiments, the present disclosure provides a method of treating or preventing dermatomyositis by administering to a subject in need thereof an effective amount of an active compound or its salt or composition as described herein.

In some embodiments, the present disclosure provides a method of treating or preventing amyotrophic lateral sclerosis by administering to a subject in need thereof an effective amount of an active compound or its salt or composition as described herein.

In some embodiments, the present disclosure provides a method of treating or preventing abdominal aortic aneurysm, hemodialysis complications, hemolytic anemia, or hemodialysis by administering to a subject in need thereof an effective amount of an active compound or its salt or composition as described herein.

In another embodiment, a method is provided for the treatment or prevention of cytokine or inflammatory reactions in response to the administration of pharmaceutical or biotherapeutic (e.g., CAR T-cell therapy or monoclonal antibody therapy) in a subject by administering an effective amount of an active compound or its salt or composition as described herein. Various types of cytokine or inflammatory reactions may occur in response to a number of factors, such as the administrations of biotherapeutics.

In some embodiments, the cytokine or inflammatory reaction is cytokine release syndrome. In some embodiments, the cytokine or inflammatory reaction is tumor lysis syndrome (which also leads to cytokine release). Symptoms of cytokine release syndrome range from fever, headache, and skin rashes to bronchospasm, hypotension, and even cardiac arrest. Severe cytokine release syndrome is described as a cytokine storm and can be fatal.

Fatal cytokine storms have been observed in response to infusion with several monoclonal antibody therapeutics. See, Abramowicz D, et al. “Release of tumor necrosis factor, interleukin-2, and gamma-interferon in serum after injection of OKT3 monoclonal antibody in kidney transplant recipients” Transplantation (1989) 47(4):606-8; Chatenoud L, et al. “In vivo cell activation following OKT3 administration. Systemic cytokine release and modulation by corticosteroids” Transplantation (1990) 49(4):697-702; and Lim L C, Koh L P, and Tan P. “Fatal cytokine release syndrome with chimeric anti-CD20 monoclonal antibody rituximab in a 71-year-old patient with chronic lymphocytic leukemia” J. Clin Oncol. (1999) 17(6):1962-3.

Also contemplated herein, is the use of an active compound or its salt or composition as described herein to mediate an adverse immune response in patients receiving bi-specific T-cell engagers (BiTE). A bi-specific T-cell engager directs T-cells to target and bind with a specific antigen on the surface of a cancer cell. For example, Blinatumomab (Amgen), a BiTE has recently been approved as a second line therapy in Philadelphia chromosome-negative relapsed or refractory acute lymphoblastic leukemia. Blinatumomab is given by continuous intravenous infusion in 4-week cycles. The use of BiTE agents has been associated with adverse immune responses, including cytokine release syndrome. The most significantly elevated cytokines in the CRS associated with ACT include IL-10, IL-6, and IFN-γ (Klinger et al., Immunopharmacologic response of patients with B-lineage acute lymphoblastic leukemia to continuous infusion of T cell-engaging CD19/CD3-bispecific BiTE antibody blinatumomab. Blood (2012) 119:6226-6233).

In another embodiment, the disorder is episcleritis, idiopathic episcleritis, anterior episcleritis, or posterior episcleritis. In some embodiments, the disorder is idiopathic anterior uveitis, HLA-B27 related uveitis, herpetic keratouveitis, Posner Schlossman syndrome, Fuch's heterochromic iridocyclitis, or cytomegalovirus anterior uveitis.

In some embodiments, the present disclosure provides a method of treating or preventing a IC-MPGN by administering to a subject in need thereof an effective amount of an active compound or its salt or composition as described herein.

In some embodiments, the present disclosure provides a method of treating or preventing a paroxysmal nocturnal hemoglobinuria (PNH) by administering to a subject in need thereof an effective amount of an active compound or its salt or composition as described herein.

In some embodiments, the present disclosure provides a method of treating or preventing a hereditary angioedema (HAE) by administering to a subject in need thereof an effective amount of an active compound or its salt or composition as described herein.

In some embodiments, the present disclosure provides a method of treating or preventing cold agglutinin disease (CAD) by administering to a subject in need thereof an effective amount of an active compound or its salt or composition as described herein.

In some embodiments, the present disclosure provides a method of treating or preventing atypical hemolytic syndrome (aHUS) by administering to a subject in need thereof an effective amount of an active compound or its salt or composition as described herein.

In some embodiments, the present disclosure provides a method of treating or preventing age-related macular degeneration (AMD) by administering to a subject in need thereof an effective amount of an active compound or its salt or composition as described herein.

In some embodiments, the present disclosure provides a method of treating or preventing rheumatoid arthritis by administering to a subject in need thereof an effective amount of an active compound or its salt or composition as described herein.

In some embodiments, the present disclosure provides a method of treating or preventing multiple sclerosis by administering to a subject in need thereof an effective amount of an active compound or its salt or composition as described herein.

In some embodiments, the present disclosure provides a method of treating or preventing myasthenia gravis by administering to a subject in need thereof an effective amount of an active compound or its salt or composition as described herein.

In some embodiments, the present disclosure provides a method of treating or preventing atypical hemolytic uremic syndrome (aHUS) by administering to a subject in need thereof an effective amount of an active compound or its salt or composition as described herein.

In yet another embodiment, the present disclosure provides a method of treating or preventing a disorder as described below by administering to a subject in need thereof an effective amount of an active compound or its salt or composition as described herein, including: vitritis, sarcoidosis, syphilis, tuberculosis, or Lyme disease; retinal vasculitis, Eales disease, tuberculosis, syphilis, or toxoplasmosis; neuroretinitis, viral retinitis, or acute retinal necrosis; varicella zoster virus, herpes simplex virus, cytomegalovirus, Epstein-Barr virus, lichen planus, or Dengue-associated disease (e.g., hemorraghic Dengue Fever); Masquerade syndrome, contact dermatitis, trauma induced inflammation, UVB induced inflammation, eczema, granuloma annulare, or acne.

In an additional embodiment, the disorder is selected from: acute myocardial infarction, aneurysm, cardiopulmonary bypass, dilated cardiomyopathy, complement activation during cardiopulmonary bypass operations, coronary artery disease, restenosis following stent placement, or percutaneous transluminal coronary angioplasty (PTCA); antibody-mediated transplant rejection, anaphylactic shock, anaphylaxis, allogenic transplant, humoral and vascular transplant rejection, graft dysfunction, graft-versus-subject disease, Graves' disease, adverse drug reactions, or chronic graft vasculopathy; allergic bronchopulmonary aspergillosis, allergic neuritis, drug allergy, radiation-induced lung injury, eosinophilic pneumonia, radiographic contrast media allergy, bronchiolitis obliterans, or interstitial pneumonia; parkinsonism-dementia complex, sporadic frontotemporal dementia, frontotemporal dementia with Parkinsonism linked to chromosome 17, frontotemporal lobar degeneration, tangle only dementia, cerebral amyloid angiopathy, cerebrovascular disorder, certain forms of frontotemporal dementia, chronic traumatic encephalopathy (CTE), Parkinson's Disease with dementia (PDD), argyrophilic grain dementia, dementia pugilistica, dementia with Lewy Bodies (DLB), or multi-infarct dementia; Creutzfeldt-Jakob disease, Huntington's disease, multifocal motor neuropathy (MMN), prion protein cerebral amyloid angiopathy, polymyositis, postencephalitic parkinsonism, subacute sclerosing panencephalitis, non-Guamanian motor neuron disease with neurofibrillary tangles, neural regeneration, and diffuse neurofibrillary tangles with calcification.

In some embodiments, the disorder is selected from: atopic dermatitis, dermatitis, dermatomyositis bullous pemphigoid, scleroderma, sclerodermatomyositis, psoriatic arthritis, pemphigus vulgaris, Discoid lupus erythematosus, cutaneous lupus, chilblain lupus erythematosus, or lupus erythematosus-lichen planus overlap syndrome; cryoglobulinemic vasculitis, mesenteric/enteric vascular disorder, peripheral vascular disorder, antineutrophil cytoplasm antibody (ANCA)-associated vasculitis (AAV), IL-2 induced vascular leakage syndrome, or immune complex vasculitis; angioedema, low platelets (HELLP) syndrome, sickle cell disease, platelet refractoriness, red cell casts, or typical or infectious hemolytic uremic syndrome (tHUS); hematuria, hemorrhagic shock, drug-induced thrombocytopenia, autoimmune hemolytic anemia (AIHA), azotemia, blood vessel and/or lymph vessel inflammation, rotational atherectomy, or delayed hemolytic transfusion reaction; British type amyloid angiopathy, Buerger's disease, bullous pemphigoid, C1q nephropathy, cancer, and catastrophic antiphospholipid syndrome. In some embodiments, the disorder is autoimmune hemolytic anemia, e.g., warm autoimmune hemolytic anemia.

In another embodiment, the disorder is selected from: wet (exudative) AMD, dry (non-exudative) AMD, chorioretinal degeneration, choroidal neovascularization (CNV), choroiditis, loss of RPE function, loss of vision (including loss of visual acuity or visual field), loss of vision from AMD, retinal damage in response to light exposure, retinal degeneration, retinal detachment, retinal dysfunction, retinal neovascularization (RNV), retinopathy of prematurity, pathological myopia, or RPE degeneration; pseudophakic bullous keratopathy, symptomatic macular degeneration related disorder, optic nerve degeneration, photoreceptor degeneration, cone degeneration, loss of photoreceptor cells, pars planitis, scleritis, proliferative vitreoretinopathy, or formation of ocular drusen; chronic urticaria, Churg-Strauss syndrome, cold agglutinin disease (CAD), corticobasal degeneration (CBD), cryoglobulinemia, cyclitis, damage of the Bruch's membrane, Degos disease, diabetic angiopathy, elevated liver enzymes, endotoxemia, epidermolysis bullosa, or epidermolysis bullosa acquisita; essential mixed cryoglobulinemia, excessive blood urea nitrogen-BUN, focal segmental glomerulosclerosis, Gerstmann-Straussler-Scheinker disease, giant cell arteritis, gout, Hallervorden-Spatz disease, Hashimoto's thyroiditis, Henoch-Schonlein purpura nephritis, or abnormal urinary sediments; hepatitis, hepatitis A, hepatitis B, hepatitis C or human immunodeficiency virus (HIV), a viral infection more generally, for example selected from Flaviviridae, Retroviruses, Coronaviridae, Poxviridae, Adenoviridae, Herpesviridae, Caliciviridae, Reoviridae, Picornaviridae, Togaviridae, Orthomyxoviridae, Rhabdoviridae, or Hepadnaviridae; Neisseria meningitidis, shiga toxin E. coli-related hemolytic uremic syndrome (STEC-HUS), hemolytic uremic syndrome (HUS); Streptococcus, and poststreptococcal glomerulonephritis.

In a further embodiment, the disorder is selected from: hyperlipidemia, hypertension, hypoalbuminemia, hypobolemic shock, hypocomplementemic urticarial vasculitis syndrome, hypophosphastasis, hypovolemic shock, idiopathic pneumonia syndrome, or idiopathic pulmonary fibrosis; inclusion body myositis, intestinal ischemia, iridocyclitis, iritis, juvenile chronic arthritis, Kawasaki's disease (arteritis), or lipiduria; membranoproliferative glomerulonephritis (MPGN) I, microscopic polyangiitis, mixed cryoglobulinemia, molybdenum cofactor deficiency (MoCD) type A, pancreatitis, panniculitis, Pick's disease, polyarteritis nodosa (PAN), progressive subcortical gliosis, proteinuria, reduced glomerular filtration rate (GFR), or renovascular disorder; multiple organ failure, multiple system atrophy (MSA), myotonic dystrophy, Niemann-Pick disease type C, chronic demyelinating diseases, or progressive supranuclear palsy; spinal cord injury, spinal muscular atrophy, spondyloarthropathies, Reiter's syndrome, spontaneous fetal loss, recurrent fetal loss, pre-eclampsia, synucleinopathy, Takayasu's arteritis, post-partum thryoiditis, thyroiditis, Type I cryoglobulinemia, Type II mixed cryoglobulinemia, Type III mixed cryoglobulinemia, ulcerative colitis, uremia, urticaria, venous gas embolus (VGE), or Wegener's granulomatosis; von Hippel-Lindau disease, histoplasmosis of the eye, hard drusen, soft drusen, pigment clumping, and photoreceptor and/or retinal pigmented epithelia (RPE) loss.

In some embodiments, an active compound or its salt or composition as described herein is useful for treating or preventing a disorder selected from autoimmune oophoritis, endometriosis, autoimmune orchitis, Ord's thyroiditis, autoimmune enteropathy, coeliac disease, Hashimoto's encephalopathy, antiphospholipid syndrome (APLS) (Hughes syndrome), aplastic anemia, autoimmune lymphoproliferative syndrome (Canale-Smith syndrome), autoimmune neutropenia, Evans syndrome, pernicious anemia, pure red cell aplasia, thrombocytopenia, adipose dolorosa (Dercum's disease), adult onset Still's disease, ankylosing spondylitis, CREST syndrome, drug-induced lupus, eosinophilic fasciitis (Shulman's syndrome), Felty syndrome, IgG4-related disease, mixed connective tissue disease (MCTD), palindromic rheumatism (Hench-Rosenberg syndrome), Parry-Romberg syndrome, Parsonage-Turner syndrome, relapsing polychondritis (Meyenburg-Altherr-Uehlinger syndrome), retroperitonial fibrosis, rheumatic fever, Schnitzler syndrome, fibromyalgia, neuromyotonia (Isaac's disease), paraneoplastic degeneration, autoimmune inner ear disease, Meniere's disease, interstitial cystitis, autoimmune pancreatitis, zika virus-related disorders, chikungunya virus-related disorders, subacute bacterial endocarditis (SBE), IgA nephropathy, IgA vasculitis, polymyalgia rheumatic, rheumatoid vasculitis, alopecia areata, autoimmune progesterone dermatitis, dermatitis herpetiformis, erythema nodosum, gestational pemphigoid, hidradenitis suppurativa, lichen sclerosus, linear IgA disease (LAD), morphea, myositis, pityriasis lichenoides et varioliformis acuta, vitiligo post-myocardial infarction syndrome (Dressier's syndrome), post-pericardiotomy syndrome, autoimmune retinopathy, Cogan syndrome, Graves opthalmopathy, ligneous conjunctivitis, Mooren's ulcer, opsoclonus myoclonus syndrome, optic neuritis, retinocochleocerebral vasculopathy (Susac's syndrome), sympathetic opthalmia, Tolosa-Hunt syndrome, interstitial lung disease, antisynthetase syndrome, Addison's disease, autoimmune polyendocrine syndrome (APS) type I, autoimmune polyendocrine syndrome (APS) type II, autoimmune polyendocrine syndrome (APS) type III, disseminated sclerosis (multiple sclerosis, pattern II), rapidly progressing glomerulonephritis (RPGN), juvenile rheumatoid arthritis, enthesitis-related arthritis, reactive arthritis (Reiter's syndrome), autoimmune hepatitis or lupoid hepatitis, primary biliary cirrhosis (PBS), primary sclerosing cholangitis, microscopic colitis, latent lupus (undifferentiated connective tissue disease (UCTD)), acute disseminated encephalomyelitis (ADEM), acute motor axonal neuropathy, anti-n-methyl-D-aspartate receptor encephalitis, Balo concentric sclerosis (Schilders disease), Bickerstaff's encephalitis, chronic inflammatory demyelinating polyneuropathy, idiopathic inflammatory demyelinating disease, Lambert-Eaton mysathenic syndrome, Oshtoran syndrome, pediatric autoimmune neuropsychiatric disorder associated with streptococcus (PANDAS), progressive inflammatory neuropathy, restless leg syndrome, stiff person syndrome, Sydenhem syndrome, transverse myelitis, lupus vasculitis, leukocytoclastic vasculitis, Microscopic Polyangiitis, polymyositis, and ischemic-reperfusion injury of the eye.

Examples of eye disorders that may be treated according to the compositions and methods disclosed herein include amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, posterior uveitis (including toxoplasmosis), pan-uveitis, an inflammatory disease of the vitreous or retina, endophthalmitis prophylaxis and treatment, macular edema, macular degeneration, age related macular degeneration, proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma, and combinations thereof.

In a further embodiment, the disorder is selected from glaucoma, diabetic retinopathy, blistering cutaneous diseases (including bullous pemphigoid, pemphigus, and epidermolysis bullosa), ocular cicatrical pemphigoid, uveitis, adult macular degeneration, diabetic retinopa retinitis pigmentosa, macular edema, diabetic macular edema, Behcet's uveitis, multifocal choroiditis, Vogt-Koyangi-Harada syndrome, imtermediate uveitis, birdshot retino-chorioditis, sympathetic ophthalmia, ocular dicatricial pemphigoid, ocular pemphigus, nonartertic ischemic optic neuropathy, postoperative inflammation, and retinal vein occlusion, and central retinal vein occulusion (CVRO).

In some embodiments, a method for the treatment of an autoimmune blistering disease in a subject is provided that includes the administration of an effective amount of an active compound or its salt or composition as described herein.

In some embodiments, a method for the treatment of bullous pemphigoid in a subject is provided that includes the administration of an effective amount of an active compound or its salt or composition as described herein.

In some embodiments, complement mediated diseases include ophthalmic diseases (including early or neovascular age-related macular degeneration and geographic atrophy), autoimmune diseases (including arthritis, rheumatoid arthritis), respiratory diseases, and cardiovascular diseases. In other embodiments, the compounds of the disclosure are suitable for use in the treatment of diseases and disorders associated with fatty acid metabolism, including obesity and other metabolic disorders.

In some embodiments, a method for the treatment of geographic atrophy in a subject is provided that includes the administration of an effective amount of an active compound or its salt or composition as described herein.

Disorders that may be treated or prevented by an active compound or its salt or composition as described herein also include, but are not limited to: hereditary angioedema, capillary leak syndrome, hemolytic uremic syndrome (HUS), neurological disorders, Guillain-Barre Syndrome, diseases of the central nervous system and other neurodegenerative conditions, glomerulonephritis (including membrane proliferative glomerulonephritis), SLE nephritis, proliferative nephritis, liver fibrosis, tissue regeneration and neural regeneration, or Barraquer-Simons Syndrome; inflammatory effects of sepsis, systemic inflammatory response syndrome (SIRS), disorders of inappropriate or undesirable complement activation, interleukin-2 induced toxicity during IL-2 therapy, inflammatory disorders, inflammation of autoimmune diseases, systemic lupus erythematosus (SLE), lupus nephritis, arthritis, immune complex disorders and autoimmune diseases, systemic lupus, or lupus erythematosus; ischemia/reperfusion injury (I/R injury), myocardial infarction, myocarditis, post-ischemic reperfusion conditions, balloon angioplasty, atherosclerosis, post-pump syndrome in cardiopulmonary bypass or renal bypass, renal ischemia, mesenteric artery reperfusion after aortic reconstruction, antiphospholipid syndrome, autoimmune heart disease, ischemia-reperfusion injuries, obesity, or diabetes; Alzheimer's dementia, stroke, schizophrenia, traumatic brain injury, trauma, Parkinson's disease, epilepsy, transplant rejection, prevention of fetal loss, biomaterial reactions (e.g. in hemodialysis, inplants), hyperacute allograft rejection, xenograft rejection, transplantation, psoriasis, burn injury, thermal injury including burns or frostbite, or crush injury; asthma, allergy, acute respiratory distress syndrome (ARDS), cystic fibrosis, adult respiratory distress syndrome, dyspnea, hemoptysis, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gases and chemicals, e.g., chlorine, phosgene, sulfur dioxide, hydrogen sulfide, nitrogen dioxide, ammonia, and hydrochloric acid), smoke injury, thermal injury (e.g., burn, freeze), bronchoconstriction, hypersensitivity pneumonitis, parasitic diseases, Goodpasture's Syndrome (anti-glomerular basement membrane nephritis), pulmonary vasculitis, Pauci-immune vasculitis, and immune complex-associated inflammation.

In some embodiments, a method for the treatment of sickle cell in a subject is provided that includes the administration of an effective amount of an active compound or its salt or composition as described herein.

In some embodiments, a method for the treatment of immune thrombocytopenic purpura (ITP), thrombotic thrombocytopenic purpura (TTP), or idiopathic thrombocytopenic purpura (ITP) in a subject is provided that includes the administration of an effective amount of an active compound or its salt or composition as described herein. In some embodiments, a method for the treatment of immune thrombocytopenic purpura (ITP).

In some embodiments, a method for the treatment of ANCA-vasculitis in a subject is provided that includes the administration of an effective amount of an active compound or its salt or composition as described herein.

In some embodiments, a method for the treatment of IgA nephropathy in a subject is provided that includes the administration of an effective amount of an active compound or its salt or composition as described herein.

In some embodiments, a method for the treatment of rapidly progressing glomerulonephritis (RPGN), in a subject is provided that includes the administration of an effective amount of an active compound or its salt or composition as described herein.

In some embodiments, a method for the treatment of lupus nephritis, in a subject is provided that includes the administration of an effective amount of an active compound or its salt or composition as described herein.

In some embodiments, a method for the treatment of hemorraghic dengue fever, in a subject is provided that includes the administration of an effective amount of an active compound or its salt or composition as described herein.

In an additional alternative embodiment, an active compound or its salt or composition as described herein is used in the treatment of an autoimmune disorder. The complement pathway enhances the ability of antibodies and phagocytic cells to clear microbes and damaged cells from the body. It is part of the innate immune system and in healthy individuals is an essential process.

Inhibiting the complement pathway will decrease the body's immune system response. Therefore, it is an object of the present disclosure to treat autoimmune disorders by administering an effective does of an active compound or its salt or composition as described herein to a subject in need thereof.

In some embodiments, the autoimmune disorder is caused by activity of the complement system. In some embodiments the autoimmune disorder is caused by activity of the alternative complement pathway. In some embodiments the autoimmune disorder is caused by activity of the classical complement pathway. In another embodiment the autoimmune disorder is caused by a mechanism of action that is not directly related to the complement system, such as the over-proliferation of T-lymphocytes or the over-production of cytokines.

Non-limiting examples of autoimmune disorders include: lupus, allograft rejection, autoimmune thyroid diseases (such as Graves' disease and Hashimoto's thyroiditis), autoimmune uveoretinitis, giant cell arteritis, inflammatory bowel diseases (including Crohn's disease, ulcerative colitis, regional enteritis, granulomatous enteritis, distal ileitis, regional ileitis, and terminal ileitis), diabetes, multiple sclerosis, pernicious anemia, psoriasis, rheumatoid arthritis, sarcoidosis, and scleroderma.

In some embodiments, an active compound or its salt or composition as described herein is used in the treatment of lupus. Non-limiting examples of lupus include lupus erythematosus, cutaneous lupus, discoid lupus erythematosus, chilblain lupus erythematosus, and lupus erythematosus-lichen planus overlap syndrome.

Lupus erythematosus is a general category of disease that includes both systemic and cutaneous disorders. The systemic form of the disease can have cutaneous as well as systemic manifestations. However, there are also forms of the disease that are only cutaneous without systemic involvement. For example, SLE is an inflammatory disorder of unknown etiology that occurs predominantly in women, and is characterized by articular symptoms, butterfly erythema, recurrent pleurisy, pericarditis, generalized adenopathy, splenomegaly, as well as CNS involvement and progressive renal failure. The sera of most patients (over 98%) contain antinuclear antibodies, including anti-DNA antibodies. High titers of anti-DNA antibodies are essentially specific for SLE. Conventional treatment for this disease has been the administration of corticosteroids or immunosuppressants.

There are three forms of cutaneous lupus: chronic cutaneous lupus (also known as discoid lupus erythematosus or DLE), subacute cutaneous lupus, and acute cutaneous lupus. DLE is a disfiguring chronic disorder primarily affecting the skin with sharply circumscribed macules and plaques that display erythema, follicular plugging, scales, telangiectasia and atrophy. The condition is often precipitated by sun exposure, and the early lesions are erythematous, round scaling papules that are 5 to 10 mm in diameter and display follicular plugging. DLE lesions appear most commonly on the cheeks, nose, scalp, and ears, but they may also be generalized over the upper portion of the trunk, extensor surfaces of the extremities, and on the mucous membranes of the mouth. If left untreated, the central lesion atrophies and leaves a scar. Unlike SLE, antibodies against double-stranded DNA (e.g., DNA-binding test) are almost invariably absent in DLE.

Diabetes can refer to either type 1 or type 2 diabetes. In some embodiments an active compound or its salt or composition as described herein is provided at an effective dose to treat a patient with type 1 diabetes. In some embodiments an active compound or its salt or composition as described herein is provided at an effective dose to treat a patient with type 2 diabetes. Type 1 diabetes is an autoimmune disease. An autoimmune disease results when the body's system for fighting infection (the immune system) attacks a part of the body. In the case of diabetes type 1, the pancreas then produces little or no insulin.

In some embodiments, the complement-mediated disease or disorder comprises transplant rejection. In some embodiments, the complement-mediated disease or disorder is antibody-mediated transplant rejection.

In certain aspects, an active compound or its salt or composition as described herein is used to treat a proliferative disorder, including, but not limited to, cancer. Targeted cancers suitable for administration of an active compound or its salt described herein include, but are not limited to, estrogen-receptor positive cancer, HER2-negative advanced breast cancer, late-line metastatic breast cancer, liposarcoma, non-small cell lung cancer, liver cancer, ovarian cancer, glioblastoma, refractory solid tumors, retinoblastoma positive breast cancer as well as retinoblastoma positive endometrial, vaginal and ovarian cancers and lung and bronchial cancers, adenocarcinoma of the colon, adenocarcinoma of the rectum, central nervous system germ cell tumors, teratomas, estrogen receptor-negative breast cancer, estrogen receptor-positive breast cancer, familial testicular germ cell tumors, HER2-negative breast cancer, HER2-positive breast cancer, male breast cancer, ovarian immature teratomas, ovarian mature teratoma, ovarian monodermal and highly specialized teratomas, progesterone receptor-negative breast cancer, progesterone receptor-positive breast cancer, recurrent breast cancer, recurrent colon cancer, recurrent extragonadal germ cell tumors, recurrent extragonadal non-seminomatous germ cell tumor, recurrent extragonadal seminomas, recurrent malignant testicular germ cell tumors, recurrent melanomas, recurrent ovarian germ cell tumors, recurrent rectal cancer, stage III extragonadal non-seminomatous germ cell tumors, stage III extragonadal seminomas, stage III malignant testicular germ cell tumors, stage III ovarian germ cell tumors, stage IV breast cancers, stage IV colon cancers, stage IV extragonadal non-seminomatous germ cell tumors, stage IV extragonadal seminoma, stage IV melanomas, stage IV ovarian germ cell tumors, stage IV rectal cancers, testicular immature teratomas, testicular mature teratomas. In particular embodiments, the targeted cancers included estrogen-receptor positive, HER2-negative advanced breast cancer, late-line metastatic breast cancer, liposarcoma, non-small cell lung cancer, liver cancer, ovarian cancer, glioblastoma, refractory solid tumors, retinoblastoma positive breast cancer as well as retinoblastoma positive endometrial, vaginal and ovarian cancers and lung and bronchial cancers, metastatic colorectal cancer, metastatic melanoma with CDK4 mutation or amplification, or cisplatin-refractory, unresectable germ cell tumors, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, fibrosarcoma, myxosarcoma, chondrosarcoma, osteosarcoma, chordoma, malignant fibrous histiocytoma, hemangiosarcoma, angiosarcoma, lymphangiosarcoma, Mesothelioma, leiomyosarcoma, rhabdomyosarcoma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, bladder cancer, and Wilms tumor, a blood disorder or a hematologic malignancy, including, but not limited to, myeloid disorder, lymphoid disorder, leukemia, lymphoma, myelodysplastic syndrome (MDS), myeloproliferative disease (MPD), mast cell disorder, and myeloma (e.g., multiple myeloma), among others, T-cell or NK-cell lymphoma, for example, but not limited to: peripheral T-cell lymphoma; anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sezary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In some embodiments, the methods described herein can be used to treat a subject, for example a human, with a lymphoma or lymphocytic or myelocytic proliferation disorder or abnormality. For example, the methods as described herein can be administered to a subject with a Hodgkin Lymphoma or a Non-Hodgkin Lymphoma. For example, the subject can have a Non-Hodgkin Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia, a Hodgkin Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL, a specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+ DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma, a leukemia, for example, an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); large granular lymphocytic leukemia; or Adult T-cell chronic leukemia. In some embodiments, the patient has an acute myelogenous leukemia, for example an undifferentiated AML (M0); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7), small cell lung cancer, retinoblastoma, HPV positive malignancies like cervical cancer and certain head and neck cancers, MYC amplified tumors such as Burkitts' Lymphoma, and triple negative breast cancer; certain classes of sarcoma, certain classes of non-small cell lung carcinoma, certain classes of melanoma, certain classes of pancreatic cancer, certain classes of leukemia, certain classes of lymphoma, certain classes of brain cancer, certain classes of colon cancer, certain classes of prostate cancer, certain classes of ovarian cancer, certain classes of uterine cancer, certain classes of thyroid and other endocrine tissue cancers, certain classes of salivary cancers, certain classes of thymic carcinomas, certain classes of kidney cancers, certain classes of bladder cancers, and certain classes of testicular cancers.

In certain aspects, an active compound or its salt as described herein can be used to preserve or prevent damage to an organ or blood product. For example, an active compound or its salt described herein can be used to prevent damage to an organ, tissue, cell product, or blood product, that has been harvested for transplantation. In some embodiments, the organ is the heart, kidney, pancreas, lung, liver, or intestine. In some embodiments, the tissue is derived from the cornea, bone, tendon, muscle, heart valve, nerve, artery or vein, or the skin. In some embodiments, the blood product is whole blood, plasma, red blood cells or reticulocytes.

In some embodiments, an active compound or its salt or composition as described herein prevents or delays the onset of at least one symptom of a complement-mediated disease or disorder in an individual. In some embodiments, an active compound or its salt or composition as described herein reduces or eliminates at least one symptom of a complement-mediated disease or disorder in an individual. Examples of symptoms include, but are not limited to, symptoms associated with autoimmune disease, cancer, hematological disease, infectious disease, inflammatory disease, ischemia-reperfusion injury, neurodegenerative disease, neurodegenerative disorder, renal disease, transplant rejection, ocular disease, vascular disease, or a vasculitis disorder. The symptom can be a neurological symptom, for example, impaired cognitive function, memory impairment, loss of motor function, etc. The symptom can also be the activity of C1s protein in a cell, tissue, or fluid of an individual. The symptom can also be the extent of complement activation in a cell, tissue, or fluid of an individual.

In some embodiments, administering an active compound or its salt or composition as described herein to an individual modulates complement activation in a cell, tissue, or fluid of an individual. In some embodiments, administration of an active compound or its salt or composition as described herein to an individual inhibits complement activation in a cell, tissue, or fluid of an individual. For example, in some embodiments, an active compound or its salt or composition as described herein, when administered in one or more doses as monotherapy or in combination therapy to an individual having a complement-mediated disease or disorder, inhibits complement activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the compounds described herein.

In some embodiments, an active compound or its salt or composition as described herein reduces C3 deposition onto red blood cells; for example, in some embodiments, an an active compound or its salt or composition as described herein reduces deposition of C3b, iC3b, etc., onto RBCs. In some embodiments, an active compound or its salt or composition as described herein inhibits complement-mediated red blood cell lysis.

In some embodiments, an active compound or its salt or composition as described herein reduces C3 deposition onto platelets; for example, in some embodiments, an active compound or its salt or composition as described herein reduces deposition of C3b, iC3b, etc., onto platelets.

In some embodiments, administering an active compound or its salt or composition as described herein results in an outcome selected from the group consisting of: (a) a reduction in complement activation; (b) an improvement in cognitive function; (c) a reduction in neuron loss; (d) a reduction in phospho-Tau levels in neurons; (e) a reduction in glial cell activation; (f) a reduction in lymphocyte infiltration; (g) a reduction in macrophage infiltration; (h) a reduction in antibody deposition, (i) a reduction in glial cell loss; (j) a reduction in oligodendrocyte loss; (k) a reduction in dendritic cell infiltration; (l) a reduction in neutrophil infiltration; (m) a reduction in red blood cell lysis; (n) a reduction in red blood cell phagocytosis; (o) a reduction in platelet phagocytosis; (p) a reduction in platelet lysis; (q) an improvement in transplant graft survival; (r) a reduction in macrophage mediated phagocytosis; (s) an improvement in vision; (t) an improvement in motor control; (u) an improvement in thrombus formation; (v) an improvement in clotting; (w) an improvement in kidney function; (x) a reduction in antibody mediated complement activation; (y) a reduction in autoantibody mediated complement activation; (z) an improvement in anemia; (aa) reduction of demyelination; (ab) reduction of eosinophilia; (ac) a reduction of C3 deposition on red blood cells (e.g., a reduction of deposition of C3b, iC3b, etc., onto RBCs); and (ad) a reduction in C3 deposition on platelets (e.g., a reduction of deposition of C3b, iC3b, etc., onto platelets); and (ae) a reduction of anaphylatoxin toxin production; (af) a reduction in autoantibody mediated blister formation; (ag) a reduction in autoantibody induced pruritis; (ah) a reduction in autoantibody induced erythematosus; (ai) a reduction in autoantibody mediated skin erosion; (aj) a reduction in red blood cell destruction due to transfusion reactions; (ak) a reduction in red blood cell lysis due to alloantibodies; (al) a reduction in hemolysis due to transfusion reactions; (am) a reduction in allo-antibody mediated platelet lysis; (an) a reduction in platelet lysis due to transfusion reactions; (ao) a reduction in mast cell activation; (ap) a reduction in mast cell histamine release; (aq) a reduction in vascular permeability; (ar) a reduction in edema; (as) a reduction in complement deposition on transplant graft endothelium; (at) a reduction of anaphylatoxin generation in transplant graft endothelium; (au) a reduction in the separation of the dermal-epidermal junction; (av) a reduction in the generation of anaphylatoxins in the dermal-epidermal junction; (aw) a reduction in alloantibody mediated complement activation in transplant graft endothelium; (ax) a reduction in antibody mediated loss of the neuromuscular junction; (ay) a reduction in complement activation at the neuromuscular junction; (az) a reduction in anaphylatoxin generation at the neuromuscular junction; (ba) a reduction in complement deposition at the neuromuscular junction; (bb) a reduction in paralysis; (bc) a reduction in numbness; (bd) increased bladder control; (be) increased bowel control; (bf) a reduction in mortality associated with autoantibodies; and (bg) a reduction in morbidity associated with autoantibodies.

In some embodiments, an active compound or its salt or composition as described herein, when administered in one or more doses to an individual having a complement-mediated disease or disorder, is effective to achieve a reduction of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: (a) complement activation; (b) decline in cognitive function; (c) neuron loss; (d) phospho-Tau levels in neurons; (e) glial cell activation; (f) lymphocyte infiltration; (g) macrophage infiltration; (h) antibody deposition, (i) glial cell loss; (j) oligodendrocyte loss; (k) dendritic cell infiltration; (l) neutrophil infiltration; (m) red blood cell lysis; (n) red blood cell phagocytosis; (o) platelet phagocytosis; (p) platelet lysis; (q) transplant graft rejection; (r) macrophage mediated phagocytosis; (s) vision loss; (t) antibody mediated complement activation; (u) autoantibody mediated complement activation; (v) demyelination; (w) eosinophilia; compared to the level or degree of the outcome in the individual before treatment with the active compound or its salt.

In some embodiments, an active compound or its salt or composition as described herein, when administered in one or more doses to an individual having a complement-mediated disease or disorder, is effective to achieve an improvement of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: a) cognitive function; b) transplant graft survival; c) vision; d) motor control; e) thrombus formation; f) clotting; g) kidney function; and h) hematocrit (red blood cell count), compared to the level or degree of the outcome in the individual before treatment with the active compound or its salt.

In some embodiments, administering an active compound or its salt or composition as described herein to an individual reduces complement activation in the individual. For example, in some embodiments, an active compound or its salt or composition as described herein, when administered in one or more doses to an individual having a complement-mediated disease or disorder, reduces complement activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the active compound or its salt.

In some embodiments, administering an active compound or its salt or composition as described herein improves cognitive function in the individual. For example, in some embodiments, an active compound described herein, when administered in one or more doses to an individual having a complement-mediated disease or disorder, improves cognitive function in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the cognitive function in the individual before treatment with the active compound or its salt.

In some embodiments, administering an active compound or its salt or composition as described herein reduces the rate of decline in cognitive function in the individual. For example, in some embodiments, an active compound or its salt, when administered in one or more doses to an individual having a complement-mediated disease or disorder, reduces the rate of decline of cognitive function in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the rate of decline in cognitive function in the individual before treatment with the active compound or its salt.

In some embodiments, administering an active compound or its salt or composition as described herein to an individual reduces neuron loss in the individual. For example, in some embodiments, an active compound or its salt, when administered in one or more doses to an individual having a complement-mediated disease or disorder, reduces neuron loss in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to neuron loss in the individual before treatment with the active compound or its salt.

In some embodiments, administering an active compound or its salt or composition as described herein to an individual reduces phospho-Tau levels in the individual. For example, in some embodiments, an active compound or its salt, when administered in one or more doses to an individual having a complement-mediated disease or disorder, reduces phospho-Tau in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the phospho-Tau level in the individual before treatment with the active compound or its salt.

In some embodiments, administering an active compound or its salt or composition as described herein to an individual reduces glial cell activation in the individual. For example, in some embodiments, an active compound or its salt, when administered in one or more doses to an individual having a complement-mediated disease or disorder, reduces glial activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to glial cell activation in the individual before treatment with the active compound or its salt. In some embodiments, the glial cells are astrocytes or microglia.

In some embodiments, administering an active compound or its salt or composition as described herein to an individual reduces lymphocyte infiltration in the individual. For example, in some embodiments, an active compound or its salt, when administered in one or more doses to an individual having a complement-mediated disease or disorder, reduces lymphocyte infiltration in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to lymphocyte infiltration in the individual before treatment with the active compound or its salt.

In some embodiments, administering an active compound or its salt or composition as described herein to an individual reduces macrophage infiltration in the individual. For example, in some embodiments, an active compound or its salt, when administered in one or more doses to an individual having a complement-mediated disease or disorder, reduces macrophage infiltration in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to macrophage infiltration in the individual before treatment with the active compound or its salt.

In some embodiments, administering an active compound or its salt or composition as described herein to an individual reduces antibody deposition in the individual. For example, in some embodiments, an active compound or its salt, when administered in one or more doses to an individual having a complement-mediated disease or disorder, reduces antibody deposition in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to antibody deposition in the individual before treatment with the active compound or its salt.

In some embodiments, administering an active compound or its salt or composition as described herein to an individual reduces anaphylatoxin (e.g., C3a, C4a, C5a) production in an individual. For example, in some embodiments, an active compound or its salt, when administered in one or more doses to an individual having a complement-mediated disease or disorder, reduces anaphylatoxin production in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the level of anaphylatoxin production in the individual before treatment with the active compound or its salt.

The present disclosure provides for use of an active compound or its salt of the present disclosure or a pharmaceutical composition comprising an active compound or its salt of the present disclosure and a pharmaceutically acceptable excipient to treat an individual having a complement-mediated disease or disorder. In some embodiments, the present disclosure provides for use of an active compound or its salt of the present disclosure to treat an individual having a complement-mediated disease or disorder. In some embodiments, the present disclosure provides for use of a pharmaceutical composition comprising an active compound or its salt of the present disclosure and a pharmaceutically acceptable excipient to treat an individual having a complement-mediated disease or disorder.

### Example 1. Non-Limiting Synthetic Examples of Compounds of the Present Disclosure

The below schemes are non-limiting examples of methods to make compounds of the present disclosure. The skilled artisan will recognize that there are various modifications that can be performed to make analogs or prepare compounds in other ways.

### Abbrevations

| | |
|---|---|
| Ac₂O | Acetic anhydride |
| AcOEt, EtOAc | ethyl acetate |
| AcOH | Acetic acid |
| AcONa | Sodium acetate |
| BH₃•Me₂S, | Borane dimethylsulfide |
| BH₃•DMS | |
| Boc₂O | Di-tert-butyl dicarbonate |
| BnBr | Benzyl bromide |
| CH₃OH, MeOH | Methanol |
| CsF | Cesium fluoride |
| CuI | Cuprous iodide |
| DAST | Diethylaminosulfur trifluoride |
| DBU | 1,8-Diazabicyclo[5.4.0]undec-7-ene |
| DCM, CH₂Cl₂ | Dichloromethane |
| DIBAL-H | Diisobutylaluminium hydride |
| DIEA, DIPEA | N,N-diisopropylethylamine |
| DMA | N,N-dimethylacetamide |
| DMAP | 4-Dimethylaminopyridine |
| DMF | N,N-dimethylformamide |
| DMSO | Dimethylsulfoxide |
| DPPA | Diphenyl phosphoryl azide |
| EtOAc | Ethylacetate |
| EtOH | Ethanol |
| FA | Formic acid |
| HATU | 1-[Bis(dimethylamino)methylene]-1H-1,2,3-tri- |
| | azolo[4,5-b]pyridinium3-oxide hexafluorophosphate |
| H₂SO₄ | Sulfuric acid |
| HCl | Hydrochloric acid |
| K₂CO₃ | Potassium carbonate |
| LiOH | Lithium hydroxide |
| LiHMDS | Lithium bis(trimethylsilyl)amide |
| MeCN | Acetonitrile |
| MeI | Methyl iodide |
| MsCl | Mesylchloride |
| MTBE | Methyl ^{t}butylether |
| NaBH₄ | Sodium borohydride |
| NaBH₃CN | Sodium cyanoborohydride |
| (n-Bu)₃SnH | Tributyltin hydride |
| Na₂SO₄ | Sodium sulfate |
| NaCl | Sodium chloride |
| NaH | Sodium hydride |
| NaHCO₃ | Sodium bicarbonate |
| NaI | Sodium iodide |
| NaOH | Sodium hydroxide |
| NBS | N-bromo succinimide |
| NH₂OH•HCl | Hydroxylamine hydrochloride |
| NH₄OH•HCl | Ammonium hydroxide hydrochloride |
| NMP | N-Methyl-2-pyrrolidone |
| Pd(OAc)₂ | Palladium acetate |
| Pd(dppf)Cl₂ | [1,1′-Bis(diphenylphosphino) ferrocene]di- |
| | chloropalladium(II) |
| Pd(PPh₃)₄ | Tetrakis(triphenylphosphine)palladium(0) |
| Pd/C | Palladium on carbon |
| PE | Petroleum ether |
| PPh₃ | Triphenylphosphine |
| PtO₂ | Platinum oxide |
| PTSA | p-Toluenesulfonic acid |
| PyBOP | benzotriazol-1-yl-oxytripyrrolidinophosphonium |
| | hexafluorophosphate |
| T3P or T₃P | Propane phosphonic acid anhydride |
| TBAF | Tetra-n-butylammonium fluoride |
| t-BuOK | Potassium tert-butoxide |
| TEA | Trimethylamine |
| Tf₂O | Trifluoromethanesulfonic anhydride |
| TFA | Trifluoroacetic acid |
| THF | Tetrahydrofuran |
| TMSBr | Bromotrimethylsilane |
| TMSCF₃ | Trifluoromethyltrimethylsilane |
| TMSCHN₂ | Trimethylsilyldiazomethane |
| TMSCN | Trimethylsilyl cyanide |
| Zn(CN)₂ | Zinc cyanide |

### General Methods

All nonaqueous reactions were performed under an atmosphere of dry argon or nitrogen gas using anhydrous solvents. The progress of reactions and the purity of target compounds were determined using one of the two liquid chromatography (LC) methods A or B disclosed herein. The structure of starting materials, intermediates, and final products was confirmed by standard analytical techniques, including NMR spectroscopy and mass spectrometry.

### LC Method A

Instrument: Waters Acquity Ultra Performance LC
Column: ACQUITY UPLC BEH C18 2.1×50 mm, 1.7 μm
Column Temperature: 40° C.
Mobile Phase: Solvent A: H₂O+0.05% FA; Solvent B: CH₃CN+0.05% FA
Flow Rate: 0.8 mL/min
Gradient: 0.24 min @15% B, 3.5 min gradient (15-85% B), then 0.5 min @85% B.
Detection: UV (210-410 nm) and MS (SQ in ES+ mode)

### Scheme 1: (1S,3S,5S)—N-((4-carbamimidoylthiophen-2-yl)methyl)-2-((chromane-6-carbonyl)glycyl)-5-methyl-2-azabicyclo[3.1.0]hexane-3-carboxamide (Compound 1)

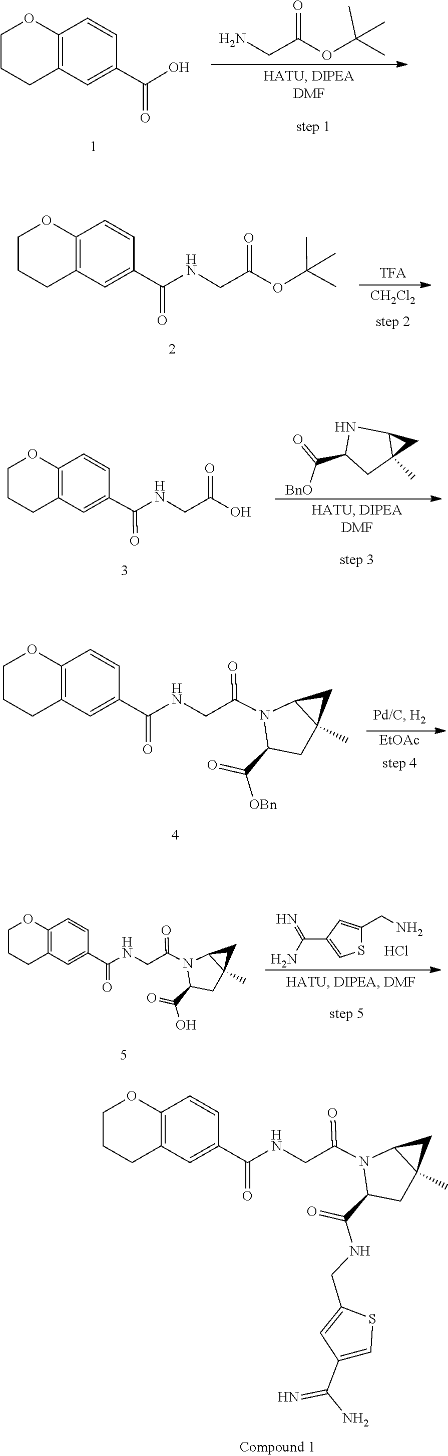

### Step 1: tert-butyl (chromane-6-carbonyl)glycinate (2)

To a solution of chromane-6-carboxylic acid, 1 (0.25 g, 1.4 mmol, 1 equiv.) in DMF (3 mL) was added tert-butyl 2-aminoacetate (0.28 g, 1.68 mmol, 1.2 equiv.), HATU (0.69 g, 1.82 mmol, 1.3 equiv.) and DIPEA (0.74 mL, 4.21 mmol, 3 equiv.). The reaction was stirred at room temperature for 1 h. Water (5 mL) was added, and the liquid was decanted, and the residue taken up in EtOAc, washed with brine, dried over Na₂SO₄, and concentrated to give compound 2 (0.3 g, 1.03 mmol, yield 73.39%) as a white solid. LC/MS (ESI) m/z: 292 (M+H)⁺.

### Step 2: (chromane-6-carbonyl)glycine (3)

TFA (0.79 mL) was added to a solution of 2 (0.3 g, 1.03 mmol, 1 equiv.) in CH₂Cl₂ (2 mL) at ice-bath temperature. The reaction was stirred at room temperature for 2 hours and then concentrated to dryness to give 3 (0.3 g, 0.85 mmol, yield 82.54%) as a white solid. LC/MS (ESI) m/z: 236 (M+H)⁺.

### Step 3: benzyl (1S,3S,5S)-2-((chromane-6-carbonyl)glycyl)-5-methyl-2-azabicyclo[3.1.0]hexane-3-carboxylate (4)

To a solution of compound 3 (0.13 g, 0.57 mmol, 1.1 equiv.) in DMF (2 mL) was added benzyl (1S,3S,5S)-5-methyl-2-azabicyclo[3.1.0]hexane-3-carboxylate (0.12 g, 0.52 mmol, 1 equiv.), HATU (0.26 g, 0.67 mmol, 1.3 equiv.) and DIPEA (0.272 mL, 1.56 mmol, 3 equiv.). The reaction was stirred at room temperature for 30 minutes. Water (5 mL) was added. The filtrate was decanted, and the residue was dissolved in EtOAc, washed with brine, dried over Na₂SO₄, and concentrated give 4 (0.18 g, 0.40 mmol, yield 70.23%) as a brown solid. LC/MS (ESI) m/z: 449 (M+H)⁺.

### Step 4: (1S,3S,5S)-2-((chromane-6-carbonyl)glycyl)-5-methyl-2-azabicyclo[3.1.0]hexane-3-carboxylic acid (5)

To a solution of 4 (0.18 g, 0.401 mmol, 1 equiv.) in EtOAc (3 mL) was added 10% Pd/C (0.043 g, 0.0401 mmol, 0.1 equiv.). The flask was evacuated and then backfilled with H₂ in a balloon. The reaction was stirred at room temperature overnight, and then concentrated to give compound 5 (0.13 g, 0.363 mmol, yield 90.38%) as a yellow solid. LC/MS (ESI) m/z: 359(M+H)⁺.

### Step 5: (1S,3S,5S)—N-((4-carbamimidoylthiophen-2-yl)methyl)-2-((chromane-6-carbonyl)glycyl)-5-methyl-2-azabicyclo[3.1.0]hexane-3-carboxamide (Compound 1)

To a solution of 5 (120 mg, 0.34 mmol, 1.0 equiv.) in DMF (2 mL) was added 5-(aminomethyl)thiophene-3-carboximidamide. HCl (71 mg, 0.37 mmol, 1.1 equiv.), HATU (140 mg, 0.37 mmol, 1.1 equiv.), and DIPEA (0.23 mL, 1.34 mmol, 4 equiv.). The reaction mixture was stirred at room temperature for 20 minutes and then purified directly by HPLC to give Compound 1 (18 mg, 0.036 mmol, yield 10.74%). LC/MS (ESI) m/z: 496 (M+H)⁺. ¹H NMR (400 MHz, DMSO-d₆) δ 8.54 (t, J=5.9 Hz, 1H), 8.51-8.39 (m, 1H), 8.34 (d, J=8.4 Hz, 1H), 7.72-7.53 (m, 1H), 7.46 (s, 1H), 6.77 (dd, J=8.6, 3.0 Hz, 1H), 6.61 (s, 1H), 4.66 (dd, J=11.3, 3.0 Hz, 1H), 4.47 (t, J=6.0 Hz, 1H), 4.40 (dd, J=12.6, 5.8 Hz, 1H), 4.36-4.25 (m, 1H), 4.18 (t, J=5.1 Hz, 3H), 4.00 (td, J=16.7, 5.6 Hz, 1H), 3.53 (q, J=7.3 Hz, 1H), 3.42 (dd, J=6.0, 2.4 Hz, 1H), 2.77 (t, J=6.3 Hz, 3H), 2.28 (t, J=12.4 Hz, 1H), 2.00 (dd, J=13.3, 3.0 Hz, 1H), 1.96-1.90 (m, 2H), 1.16 (s, 3H), 0.72-0.57 (m, 2H).

### Scheme 2: Synthesis of (1S,3S,5R)—N—((R)-1-(4-carbamimidoylthiophen-2-yl)ethyl)-2-((4-phenoxybutanoyl)glycyl)-5-((2-(2-(((2S,3S,4S,5S)-2,3,4,5-tetrahydroxyhexyl) amino)acetamido)ethoxy)methyl)-2-azabicyclo[3.1.0]hexane-3-carboxamide (Compound 2)

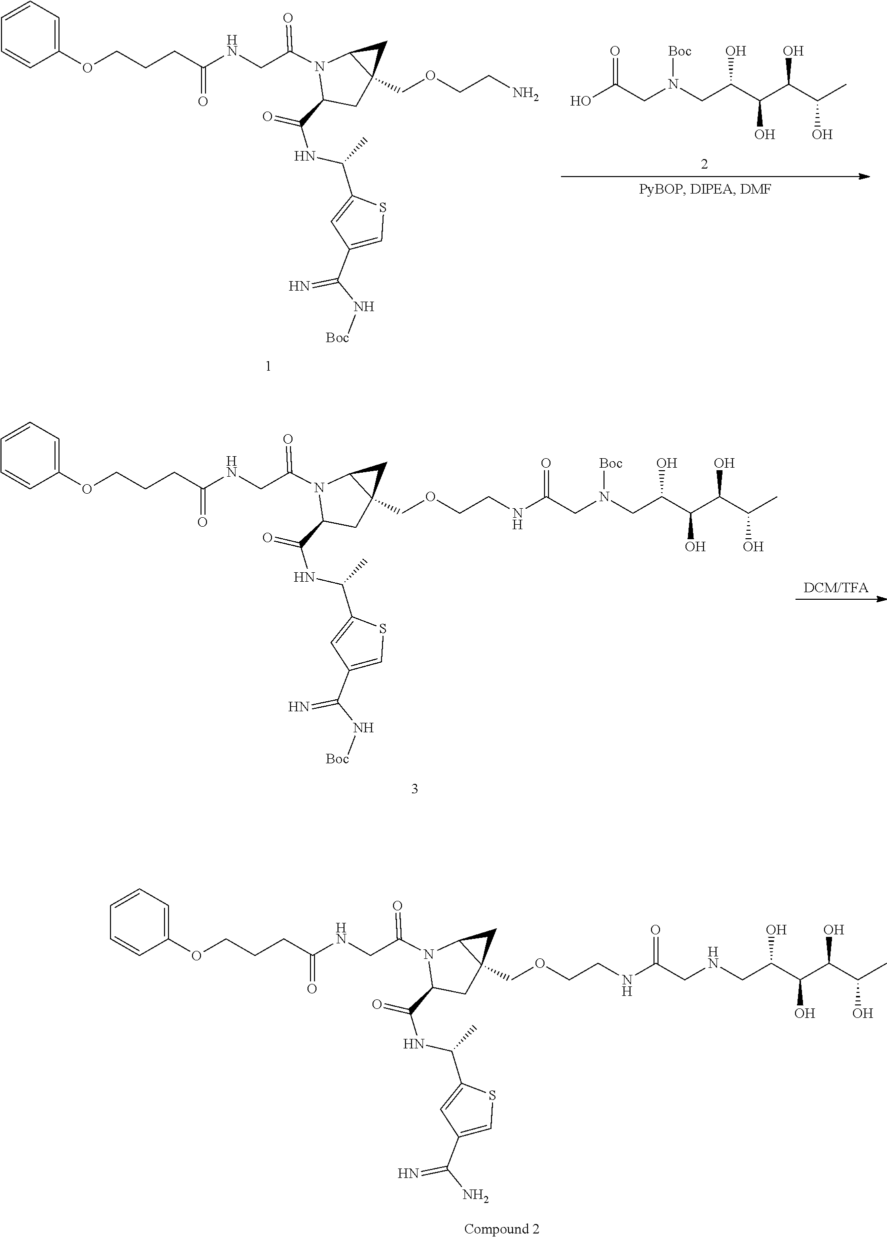 [SMILES: C[C@@H](NC(=O)[C@@H]1C[C@]2(COCCN)C[C@@H]2N1C(=O)CNC(=O)CCCOc1ccccc1)c1cc(C(=N)NC(=O)OC(C)(C)C)cs1 ; C[C@H](O)[C@H](O)[C@@H](O)[C@@H](O)CN(CC(=O)O)C(=O)OC(C)(C)C ; C[C@H](O)[C@H](O)[C@@H](O)[C@@H](O)CN(CC(=O)NCCOC[C@@]12C[C@@H]1N(C(=O)CNC(=O)CCCOc1ccccc1)[C@H](C(=O)N[C@H](C)c1cc(C(=N)NC(=O)OC(C)(C)C)cs1)C2)C(=O)OC(C)(C)C ; C[C@H](O)[C@H](O)[C@@H](O)[C@@H](O)CNCC(=O)NCCOC[C@@]12C[C@@H]1N(C(=O)CNC(=O)CCCOc1ccccc1)[C@H](C(=O)N[C@H](C)c1cc(C(=N)N)cs1)C2]

### Step 1: tert-butyl (2-((2-(((1S,3S,5R)-3-(((R)-1-(4-(N-(tert-butoxycarbonyl)carbamimidoyl) thiophen-2-yl)ethyl)carbamoyl)-2-((4-phenoxybutanoyl)glycyl)-2-azabicyclo[3.1.0]hexan-5-yl)methoxy)ethyl)amino)-2-oxoethyl)((2S,3S,4S,5S)-2,3,4,5-tetrahydroxyhexyl)carbamate (3)

To a mixture of tert-butyl ((5-((R)-1-((1S,3S,5R)-5-((2-aminoethoxy)methyl)-2-((4-phenoxybutanoyl)glycyl)-2-azabicyclo[3.1.0]hexane-3-carboxamido)ethyl)thiophen-3-yl) (imino)methyl)carbamate (40 mg, 0.06 mmol) and N-(tert-butoxycarbonyl)-N-((2S,3S,4S,5S)-2,3,4,5-tetrahydroxyhexyl)glycine (38.5 mg, 0.12 mmol) in DMF (3.0 mL) was added DIPEA (0.05 mL, 0.30 mmol) and PyBOP (47 mg, 0.09 mmol) at 0° C. under N₂ atmosphere and the mixture was stirred at 25° C. for 1 hour. The mixture was concentrated to dryness under reduced pressure. The residue was purified by prep-HPLC to give tert-butyl (2-((2-(((1 S,3S,5R)-3-(((R)-1-(4-(N-(tert-butoxycarbonyl)carbamimidoyl)thiophen-2-yl)ethyl)carbamoyl)-2-((4-phenoxybutanoyl)glycyl)-2-azabicyclo[3.1.0]hexan-5-yl)methoxy)ethyl)amino)-2-oxoethyl) ((2S,3S,4S,5S)-2,3,4,5-tetrahydroxyhexyl)carbamate (15 mg, yield 25.9%) as a white solid. LC/MS (ESI) m/z: 976 (M+H)⁺.

### Step 2: (1S,3S,5R)—N—((R)-1-(4-carbamimidoylthiophen-2-yl)ethyl)-2-((4-phenoxybutanoyl) glycyl)-5-((2-(2-(((2S,3S,4S,5S)-2,3,4,5-tetrahydroxyhexyl)amino)acetamido)ethoxy)methyl)-2-azabicyclo[3.1.0]hexane-3-carboxamide (Compound 2)

A solution of tert-butyl (2-((2-(((1 S,3S,5R)-3-(((R)-1-(4-(N-(tert-butoxycarbonyl)carbamimidoyl) thiophen-2-yl)ethyl)carbamoyl)-2-((4-phenoxybutanoyl)glycyl)-2-azabicyclo[3.1.0]hexan-5-yl)methoxy)ethyl)amino)-2-oxoethyl)((2S,3S,4S,5S)-2,3,4,5-tetrahydroxyhexyl)carbamate (15 mg, 0.02 mmol) in DCM (2 mL) was added TFA (1 mL) at 0° C. and the solution was stirred at room temperature for 1 hour. The reaction mixture was concentrated to dryness under reduced pressure, co-evaporated with DCM and dried under vacuum. The residue was purified by prep-HPLC to give (1S,3S,5R)—N—((R)-1-(4-carbamimidoylthiophen-2-yl)ethyl)-2-((4-phenoxybutanoyl)glycyl)-5-((2-(2-(((2S,3S,4S,5S)-2,3,4,5-tetrahydroxyhexyl)amino)acetamido) ethoxy)methyl)-2-azabicyclo[3.1.0]hexane-3-carboxamide (5.0 mg, yield 33.3%) as a white solid. ¹H-NMR (400 MHz, CD₃OD) δ 8.23 (d, J=1.4 Hz, 1H), 7.52 (s, 1H), 7.25 (dd, J=10.7, 5.3 Hz, 2H), 6.90 (dd, J=7.9, 6.5 Hz, 3H), 5.21 (q, J=7.0 Hz, 1H), 4.60 (s, 2H), 4.30 (d, J=16.7 Hz, 1H), 4.07 (d, J=16.7 Hz, 1H), 4.01 (t, J=6.2 Hz, 2H), 3.93 (s, 1H), 3.81 (t, J=11.7 Hz, 4H), 3.63-3.52 (m, 4H), 3.48-3.41 (m, 4H), 3.37 (d, J=14.1 Hz, 1H), 3.14 (t, J=10.0 Hz, 1H), 2.67 (t, J=13.5 Hz, 1H), 2.49 (t, J=7.4 Hz, 2H), 2.17-2.06 (m, 3H), 1.60 (dd, J=20.9, 7.0 Hz, 3H), 1.45-1.21 (m, 5H), 1.01 (t, J=5.8 Hz, 1H). LC/MS (ESI) m/z: 776 (M+H)⁺.

### Scheme 3: Synthesis of (2S,4R)—N-((4-carbamimidoylthiophen-2-yl)methyl)-4-(difluoromethoxy)-1-((4-(4-fluorophenoxy)benzoyl)glycyl)pyrrolidine-2-carboxamide (Compound 3)

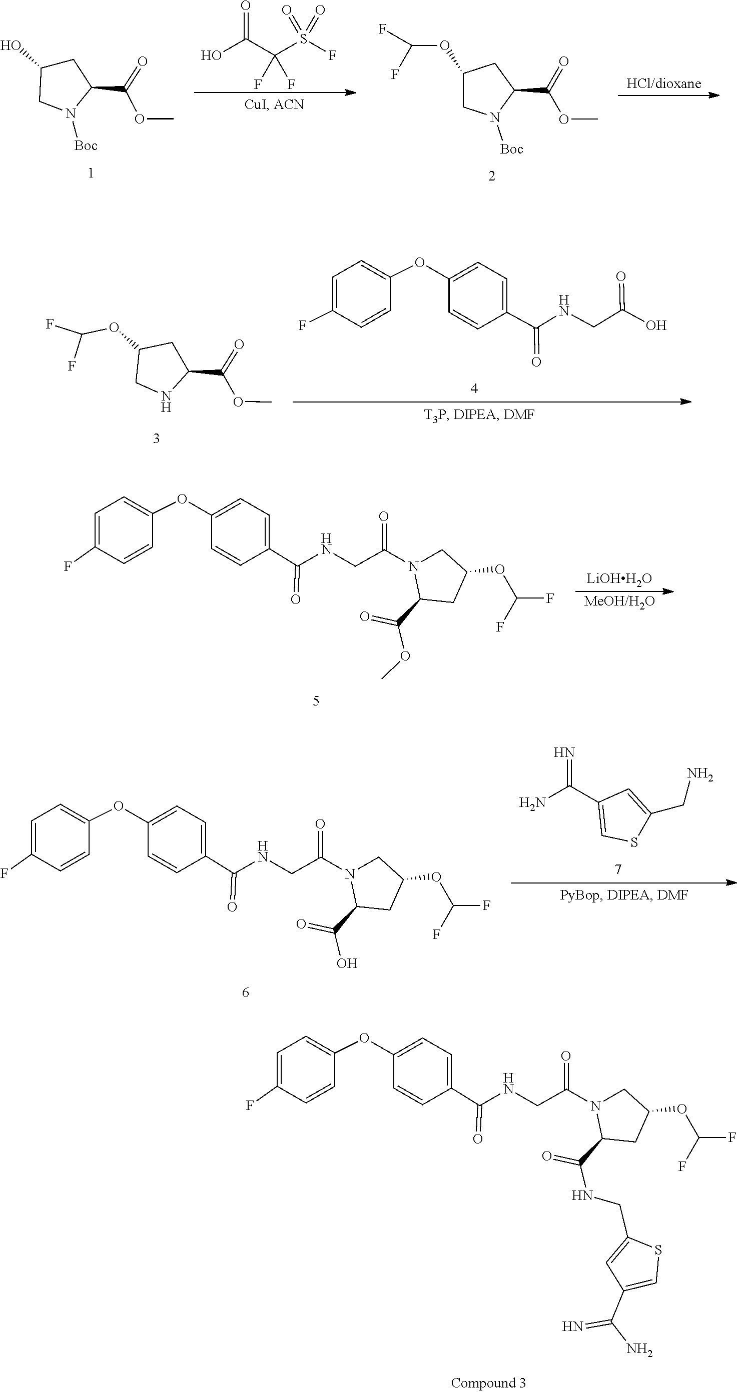

### Step 1: 1-(tert-butyl) 2-methyl (2S,4R)-4-(difluoromethoxy) pyrrolidine-1,2-dicarboxylate (2)

To a mixture of 1-(tert-butyl) 2-methyl (2S,4R)-4-hydroxypyrrolidine-1,2-dicarboxylate (5.0 g, 20.40 mmol) and CuI (776 mg, 4.08 mmol) in CH₃CN (66 mL) was added a solution of 2,2-difluoro-2-(fluorosulfonyl)acetic acid (4.4 g, 24.48 mmol) in CH₃CN (20 mL) drop-wisely at 50° C. under N₂ atmosphere and the mixture was stirred at 50° C. for 2 hours. The mixture was diluted with water and extracted with ethyl acetate twice. The combined organic layers were dried over anhydrous Na₂SO₄, filtered, and concentrated to dryness under reduced pressure. The residue was purified by column chromatography on silica gel (PE:ethyl acetate=10:1) to give 1-(tert-butyl) 2-methyl (2S,4R)-4-(difluoromethoxy) pyrrolidine-1,2-dicarboxylate (4.0 g, yield 66.7%) as a colorless oil. LC/MS (ESI) m/z: 296 (M+H)⁺.

### Step 2: methyl (2S,4R)-4-(difluoromethoxy)pyrrolidine-2-carboxylate hydrocloride (3)

A solution of 1-(tert-butyl) 2-methyl (2S,4R)-4-(difluoromethoxy)pyrrolidine-1,2-dicarboxylate (400 mg, 1.35 mmol) in HCl/1,4-dioxane (5 mL, 4M) was stirred at room temperature for 1 hour. The reaction mixture was concentrated to dryness under reduced pressure, co-evaporated with DCM and dried under vacuum to give methyl (2S,4R)-4-(difluoromethoxy)pyrrolidine-2-carboxylate (320 mg, yield 100%) as a light-yellow solid, which was used directly in the next step without further purification. LC/MS (ESI) m/z: 196 (M+H)⁺.

### Step 3: methyl (2S,4R)-4-(difluoromethoxy)-1-((4-(4-fluorophenoxy)benzoyl)glycyl) pyrrolidine-2-carboxylate (5)

To a mixture of methyl (2S,4R)-4-(difluoromethoxy)pyrrolidine-2-carboxylate hydrochloride (121 mg, 0.52 mmol) and (4-(4-fluorophenoxy)benzoyl)glycine (100 mg, 0.35 mmol) in DMF (3.0 mL) was added DIPEA (0.34 mL, 3.12 mmol) and T₃P (660 mg, 1.56 mmol, 50% wt in ethyl acetate) at 0° C. under N₂ atmosphere and the mixture was stirred at 25° C. for 16 hours. The mixture was diluted with water and extracted with ethyl acetate twice. The combined organic layers were dried over anhydrous Na₂SO₄, filtered, and concentrated to dryness under reduced pressure. The residue was purified by column chromatography on silica gel (DCM:MeOH=97:3) to give methyl (2S,4R)-4-(difluoromethoxy)-1-((4-(4-fluorophenoxy)benzoyl)glycyl) pyrrolidine-2-carboxylate (150 mg, yield 93.2%) as a light-yellow oil. LC/MS (ESI) m/z: 467 (M+H)⁺.

### Step 4: (2S,4R)-4-(difluoromethoxy)-1-((4-(4-fluorophenoxy)benzoyl)glycyl)pyrrolidine-2-carboxylic acid (6)

To a solution of methyl (2S,4R)-4-(difluoromethoxy)-1-((4-(4-fluorophenoxy)benzoyl)glycyl) pyrrolidine-2-carboxylate (150 mg, 0.32 mmol) in MeOH (3 mL) and water (0.6 mL) was added lithium hydroxide (20 mg, 0.48 mmol) at 0° C. and the mixture was stirred at room temperature for 2 hours. The mixture was acidified with 1 N aq·HCl to pH˜3, extracted with CHCl₃/i-PrOH(3/1, v/v) five times. The combined organic layers were washed with brine, dried over anhydrous Na₂SO₄, filtered, and concentrated to dryness under reduced pressure to give (2S,4R)-4-(difluoromethoxy)-1-((4-(4-fluorophenoxy)benzoyl)glycyl)pyrrolidine-2-carboxylic acid (120 mg, yield 82.8%) as a white solid, which was used directly in the next step. LC/MS (ESI) (m/z): 453 (M+H)⁺.

### Step 5: (2S,4R)—N-((4-carbamimidoylthiophen-2-yl)methyl)-4-(difluoromethoxy)-1-((4-(4-fluorophenoxy)benzoyl)glycyl)pyrrolidine-2-carboxamide (Compound 3)

To a mixture of (2S,4R)-4-(difluoromethoxy)-1-((4-(4-fluorophenoxy)benzoyl)glycyl) pyrrolidine-2-carboxylic acid (50 mg, 0.11 mmol) and 5-(aminomethyl)thiophene-3-carboximidamide (26 mg, 0.16 mmol) in DMF (3.0 mL) was added DIPEA (0.09 mL, 0.66 mmol) and PyBOP (86 mg, 0.16 mmol) at 0° C. under N₂ atmosphere and the mixture was stirred at 25° C. for 16 hours. The mixture was concentrated to dryness under reduced pressure. The residue was purified by prep-TLC (DCM:MeOH=5:1) and further purified by prep-HPLC to give Compound 3 (20 mg, yield 30.8%) as a white solid. ¹H NMR (400 MHz, CD₃OD) δ 8.53 (s, 1H), 8.23 (dd, J=17.0, 1.6 Hz, 1H), 7.85 (d, J=8.8 Hz, 2H), 7.46 (t, J=17.8 Hz, 1H), 7.26-7.12 (m, 2H), 7.09 (ddd, J=6.8, 5.3, 2.9 Hz, 2H), 7.00 (t, J=5.8 Hz, 2H), 6.49 (td, J=74.4, 16.7 Hz, 1H), 5.00 (d, J=3.5 Hz, 1H), 4.73-4.54 (m, 3H), 4.33-4.01 (m, 2H), 3.94 (ddd, J=34.0, 16.7, 7.4 Hz, 2H), 2.49 (ddd, J=31.9, 28.1, 12.3 Hz, 1H), 2.40-2.17 (m, 1H). LC/MS (ESI) m/z: 590 (M+H)⁺.

### Scheme 4: (1S,3S,5S)—N-((4-carbamimidoylthiophen-2-yl)methyl)-2-((9,9-difluoro-9H-fluorene-3-carbonyl)glycyl)-5-methyl-2-azabicyclo[3.1.0]hexane-3-carboxamide(Compound 4)

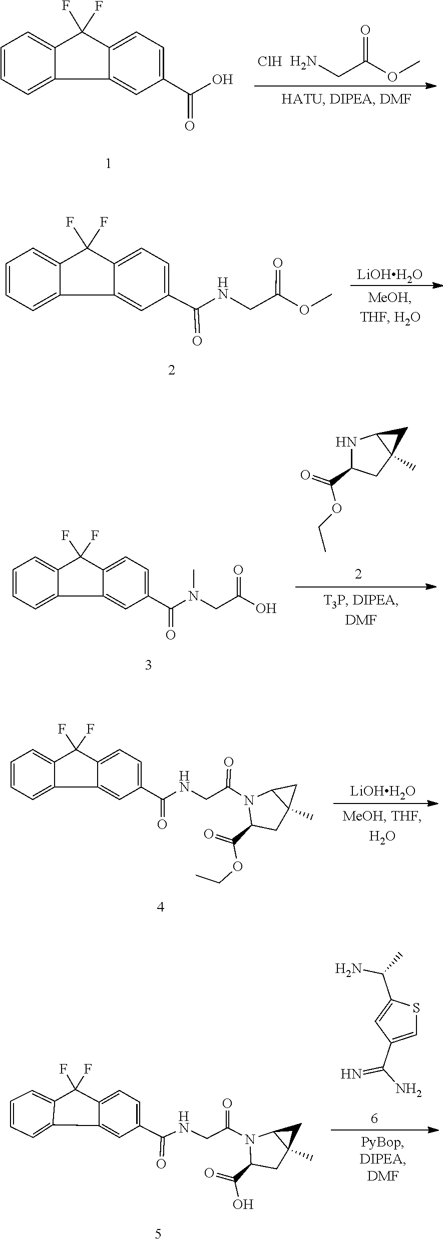

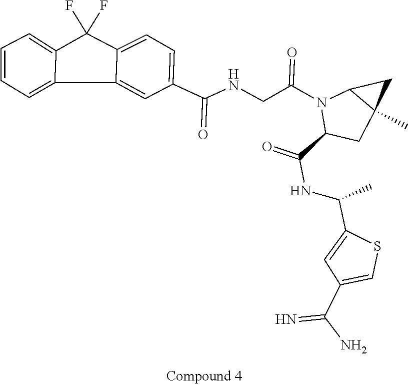

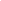

### Step 1: methyl (9,9-difluoro-9H-fluorene-3-carbonyl)glycinate (2)

To a mixture of methyl (9,9-difluoro-9H-fluorene-3-carbonyl)glycinate (150 mg, 0.61 mmol) and methyl glycinate hydrochloride (92 mg, 0.73 mmol) in DMF (2 mL) was added DIPEA (472 mg, 3.65 mmol) and HATU (347 mg, 0.91 mmol) at 0° C. The reaction mixture was stirred under N₂ atmosphere at room temperature overnight. The mixture was quenched with saturated aq.NaHCO₃ solution. The aqueous layer was extracted with ethyl acetate twice and the combined organic layers were washed with brine, dried over anhydrous Na₂SO₄, filtered, and concentrated to dryness under reduced pressure. The residue was purified by flash chromatography on silica gel (petroleum ether:ethyl acetate=1:1) to afford the title methyl (9,9-difluoro-9H-fluorene-3-carbonyl)glycinate (150 mg, yield 77.6%) as a white solid. LC/MS (ESI) m/z:318 (M+H)⁺.

### Step 2: (9,9-difluoro-9H-fluorene-3-carbonyl)glycine (3)

To a solution of methyl (9,9-difluoro-9H-fluorene-3-carbonyl)glycinate (150 mg, 0.47 mmol) in CH₃OH (1.5 mL), THF(0.5 ml) and water (0.5 mL) was added LiOH·H₂O (60 mg, 1.42 mmol) at 0° C. and the mixture was stirred under N₂ atmosphere at room temperature for 1 hour. The mixture was acidified with 1 N aq·HCl solution to pH3 and extracted with ethyl acetate twice. The combined organic layers were concentrated to dryness under reduced pressure to give (9,9-difluoro-9H-fluorene-3-carbonyl)glycine (140 mg, yield 98.0%) as a white solid, which was used directly in next step without further purification. LC/MS (ESI) (m/z): 304(M+H)⁺.

### Step 3: ethyl (1S,3S,5S)-2-((9,9-difluoro-9H-fluorene-3-carbonyl)glycyl)-5-methyl-2-azabicyclo[3.1.0]hexane-3-carboxylate (4)

To a mixture of (9,9-difluoro-9H-fluorene-3-carbonyl)glycine (60 mg, 0.20 mmol) and ethyl (1S,3S,5S)-5-methyl-2-azabicyclo[3.1.0]hexane-3-carboxylate (50 mg, 0.30 mmol) in DMF (1 mL) was added DIPEA (153 mg, 1.19 mmol) and T₃P (189 mg, 0.59 mmol) at 0° C. under N₂ atmosphere and the reaction mixture was stirred at room temperature for 1 hour. The mixture was quenched with saturated aq.NaHCO₃ solution. The aqueous layer was extracted with ethyl acetate twice and the combined organic layers were washed with brine, dried over anhydrous Na₂SO₄, filtered, and concentrated to dryness under reduced pressure. The residue was purified by flash chromatography on silica gel (petroleum ether:ethyl acetate=3:1) to afford the title ethyl (1S,3S,5S)-2-((9,9-difluoro-9H-fluorene-3-carbonyl)glycyl)-5-methyl-2-azabicyclo[3.1.0]hexane-3-carboxylate (50 mg, yield 55.6%) as a white solid. LC/MS (ESI) m/z:455 (M+H)⁺.

### Step 4: (1S,3S,5S)-2-((9,9-difluoro-9H-fluorene-3-carbonyl)glycyl)-5-methyl-2-azabicyclo[3.1.0]hexane-3-carboxylic acid (5)

To a solution of ethyl (1 S,3S,5S)-2-((9,9-difluoro-9H-fluorene-3-carbonyl)glycyl)-5-methyl-2-azabicyclo[3.1.0]hexane-3-carboxylate (50 mg, 0.11 mmol) in CH₃OH (1.2 mL), THF (0.4 ml) and water (0.4 mL) was added LiOH·H₂O (14 mg, 0.33 mmol) at 0° C. and the mixture was stirred under N₂ atmosphere at room temperature for 1 hour. The mixture was acidified with 1 N aq·HCl solution to pH<3 and extracted with ethyl acetate twice. The combined organic layers were concentrated to dryness under reduced pressure to give (1 S,3S,5S)-2-((9,9-difluoro-9H-fluorene-3-carbonyl)glycyl)-5-methyl-2-azabicyclo[3.1.0]hexane-3-carboxylic acid (40 mg, 83.3% yield) as a white solid, which was used directly in the next step without further purification. LC/MS (ESI) (m/z): 427(M+H)⁺.

### Step 5: (1S,3S,5S)—N—((R)-1-(4-carbamimidoylthiophen-2-yl)ethyl)-2-((9,9-difluoro-9H-fluorene-3-carbonyl)glycyl)-5-methyl-2-azabicyclo[3.1.0]hexane-3-carboxamide (Compound 4)

To a mixture of (1 S,3S,5S)-2-((9,9-difluoro-9H-fluorene-3-carbonyl)glycyl)-5-methyl-2-azabicyclo[3.1.0]hexane-3-carboxylic acid (20 mg, 0.047 mmol) and (R)-5-(1-aminoethyl)thiophene-3-carboximidamide (12 mg, 0.070 mmol) in DMF (1 mL) was added DIPEA (36 mg, 0.28 mmol) and PyBop (36 mg, 0.070 mmol) at 0° C. under N₂ atmosphere and the reaction mixture was stirred at room temperature for 1 hour. The mixture was quenched with saturated aq.NaHCO₃ solution. The aqueous layer was extracted with ethyl acetate twice and the combined organic layers were washed with brine, dried over anhydrous Na₂SO₄, filtered, and concentrated to dryness under reduced pressure. The residue was purified by flash chromatography on silica gel (dichloromethane:methanol=10:1) and prep-HPLC to afford Compound 4 (3 mg, yield 11.1%). ¹H NMR (400 MHz, CD₃OD) δ 8.55 (s, 1H), 8.28-8.13 (m, 2H), 7.90-7.85 (m, 1H), 7.74 (dd, J=13.9, 8.0 Hz, 2H), 7.66 (d, J=7.3 Hz, 1H), 7.58 (t, J=7.2 Hz, 1H), 7.50 (s, 1H), 7.45 (t, J=7.4 Hz, 1H), 5.28-5.17 (m, 1H), 4.48 (d, J=16.6 Hz, 1H), 4.31 (d, J=16.4 Hz, 1H), 3.42 (dd, J=6.0, 2.4 Hz, 1H), 2.19 (s, 1H), 2.03 (d, J=5.7 Hz, 2H), 1.58 (d, J=7.0 Hz, 3H), 1.32 (s, 3H), 1.25 (dd, J=5.5, 2.6 Hz, 1H), 0.81 (t, J=5.8 Hz, 1H). LC/MS (ESI) m/z:578 (M+H)⁺.

### Scheme 5: (2S,4R)—N—((R)-1-(4-carbamimidoylthiophen-2-yl)ethyl)-4-(difluoromethoxy)-1-((4-(4-(trifluoromethyl)phenoxy)benzoyl)glycyl)pyrrolidine-2-carboxamide (Compound 5)

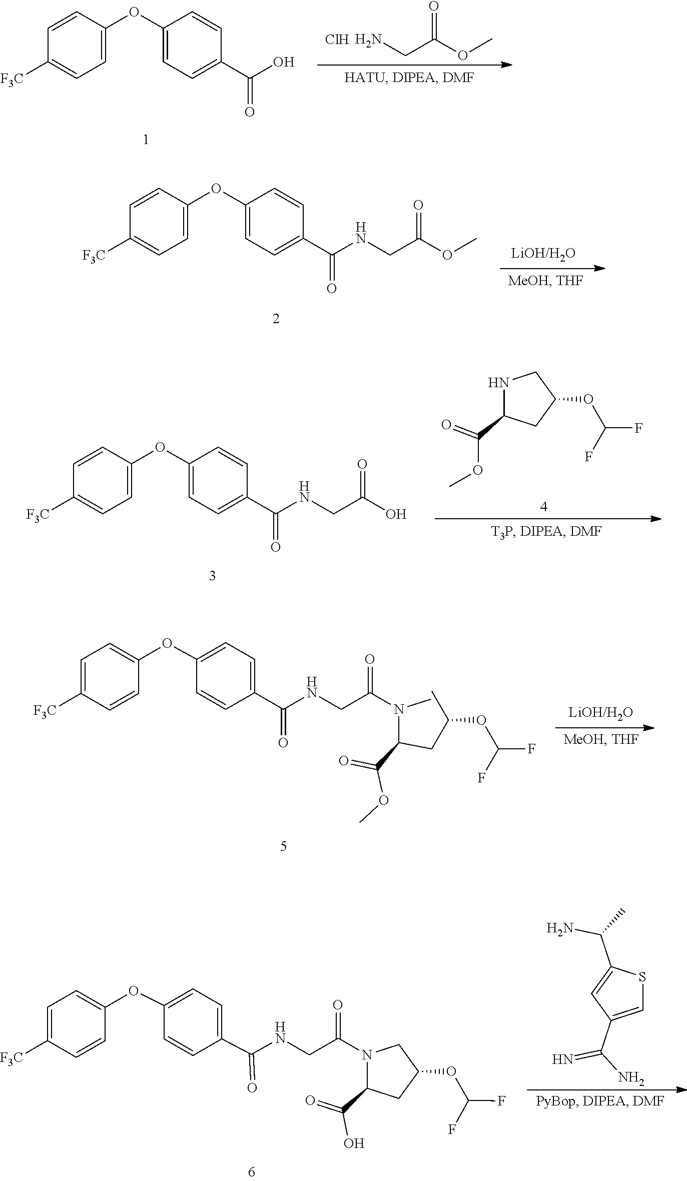

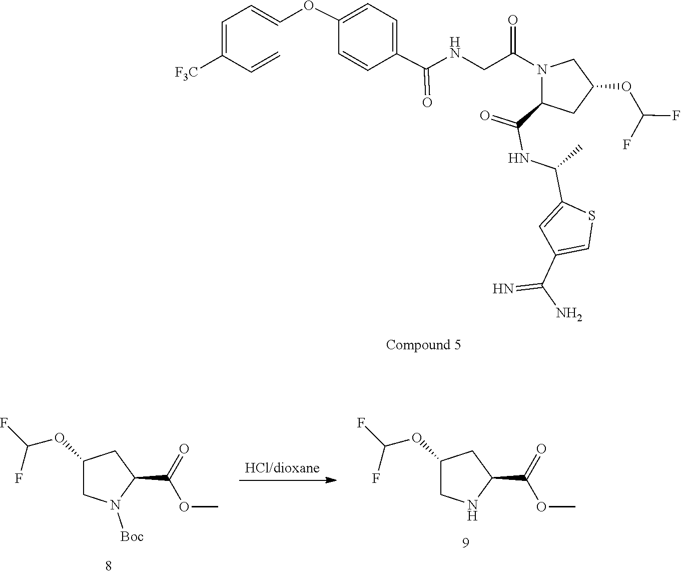 [SMILES: C=C/C(=C\C=C\Oc1ccc(C(=O)NCC(=O)N2C[C@H](OC(F)F)C[C@H]2C(=O)N[C@H](C)c2cc(C(=N)N)cs2)cc1)C(F)(F)F ; COC(=O)[C@@H]1C[C@@H](OC(F)F)CN1C(=O)OC(C)(C)C ; COC(=O)[C@@H]1C[C@@H](OC(F)F)CN1]

### Step 1: methyl (2S,4R)-4-(difluoromethoxy)pyrrolidine-2-carboxylate (9)

To a solution of 2-benzyl 1-(tert-butyl) (2S,4S)-4-(o-tolyl)pyrrolidine-1,2-dicarboxylate (80 mg, 0.30 mmol) in HCl/1,4-dioxane (1 mL) was stirred at room temperature for 1 hour. The reaction mixture was concentrated to dryness under reduced pressure, diluted with DCM again and dried under vacuum to give methyl (2S,4R)-4-(difluoromethoxy)pyrrolidine-2-carboxylate (46 mg, yield 90%), which was used directly in the next step without further purification. LC/MS (ESI) m/z: 170 (M+H)⁺.

Step 2: methyl (4-(4-(trifluoromethyl)phenoxy)benzoyl)glycinate (2)

To a mixture of 1-(tert-butyl) 2-methyl (2S,4R)-4-(difluoromethoxy)pyrrolidine-1,2-dicarboxylate (1.0 g, 3.54 mmol) and methyl glycinate hydrochloride (668 mg, 5.32 mmol) in DMF (10 mL) was added DIPEA (2.75 g, 21.26 mmol) and HATU (2.02 g, 5.32 mmol) at 0° C. under N₂ atmosphere and the reaction mixture was stirred at room temperature for 1 hour. The mixture was diluted with water and extracted with ethyl acetate twice. The combined organic layers were washed with brine, dried over anhydrous Na₂SO₄, filtered, and concentrated to dryness under reduced pressure. The residue was purified by flash chromatography on silica gel (petroleum ether:ethyl acetate=1:1) to afford the title methyl (4-(4-(trifluoromethyl)phenoxy)benzoyl)glycinate (1.1 g, yield 87.8%) as a white solid. LC/MS (ESI) m/z:354 (M+H)⁺.

### Step 3: (4-(4-(trifluoromethyl)phenoxy)benzoyl)glycine (3)

To a mixture of methyl (4-(4-(trifluoromethyl)phenoxy)benzoyl)glycinate (1.1 g, 3.11 mmol) in CH₃OH (1.5 mL), THF(0.5 ml) and water (0.5 mL) was added LiOH·H₂O (356 mg, 8.49 mmol) at 0° C. and the mixture was stirred under N₂ atmosphere at room temperature for 1 hour. The mixture was acidified with 1 N aq·HCl solution to pH<3 and extracted with ethyl acetate twice. The combined organic layers were concentrated to dryness under reduced pressure to afford (4-(4-(trifluoromethyl)phenoxy)benzoyl)glycine (988 mg, 93.6% yield) as a white solid, which was used directly in next step without further purification. LC/MS (ESI) (m/z): 340(M+H)⁺.

### Step 4: methyl (2S,4R)-4-(difluoromethoxy)-1-((4-(4-(trifluoromethyl)phenoxy)benzoyl) glycyl)pyrrolidine-2-carboxylate (5)

To a mixture of (4-(4-(trifluoromethyl)phenoxy)benzoyl)glycine (87 mg, 0.26 mmol) and methyl (2S,4R)-4-(difluoromethoxy)pyrrolidine-2-carboxylate (50 mg, 0.26 mmol) in DMF (1 mL) was added DIPEA (198 mg, 1.54 mmol) and T₃P (244 mg, 0.77 mmol) at 0° C. under N₂ atmosphere and the reaction mixture was stirred at room temperature for 1 hour. The mixture was diluted with water and extracted with ethyl acetate twice. The combined organic layers were washed with brine, dried over anhydrous Na₂SO₄, filtered, and concentrated to dryness under reduced pressure. The residue was purified by flash chromatography on silica gel (petroleum ether:ethyl acetate=1:1) to afford the title methyl (2S,4R)-4-(difluoromethoxy)-1-((4-(4-(trifluoromethyl)phenoxy)benzoyl)glycyl)pyrrolidine-2-carboxylate (82 mg, yield 62.0%) as a white solid. LC/MS (ESI) m/z:517 (M+H)⁺.

### Step 5: (2S,4R)-4-(difluoromethoxy)-1-((4-(4-(trifluoromethyl)phenoxy)benzoyl)glycyl) pyrrolidine-2-carboxylic acid (6)

To a mixture of methyl (2S,4R)-4-(difluoromethoxy)-1-((4-(4-(trifluoromethyl)phenoxy) benzoyl)glycyl)pyrrolidine-2-carboxylate (82 mg, 0.16 mmol) in CH₃OH (1.5 mL), THF(0.5 ml) and water (0.5 mL) was added LiOH·H₂O (19 mg, 0.48 mmol) at 0° C. and the mixture was stirred under N₂ atmosphere at room temperature for 1 hour. The mixture was acidified with 1 N aq·HCl solution to pH<3 and extracted with ethyl acetate twice. The combined organic layers were concentrated to dryness under reduced pressure to afford (2S,4R)-4-(difluoromethoxy)-1-((4-(4-(trifluoromethyl)phenoxy)benzoyl)glycyl)pyrrolidine-2-carboxylic acid as a white solid, which was used directly in next step without further purification. LC/MS (ESI) (m/z): 503(M+H)⁺.

### Step 6: (2S,4R)—N—((R)-1-(4-carbamimidoylthiophen-2-yl)ethyl)-4-(difluoromethoxy)-1-((4-(4-(trifluoromethyl)phenoxy)benzoyl)glycyl)pyrrolidine-2-carboxamide (Compound 5)

To a mixture of (2S,4R)-4-(difluoromethoxy)-1-((4-(4-(trifluoromethyl)phenoxy) benzoyl)glycyl)pyrrolidine-2-carboxylic acid (52 mg, 0.10 mmol) and (R)-5-(1-aminoethyl)thiophene-3-carboximidamide (17 mg, 0.10 mmol) in DMF (1 mL) was added DIPEA (78 mg, 0.60 mmol) and PyBop (78 mg, 0.15 mmol) at 0° C. under N₂ atmosphere and the reaction mixture was stirred at room temperature for 1 hour. The mixture was diluted with water and extracted with ethyl acetate twice. The combined organic layers were washed with brine, dried over anhydrous Na₂SO₄, filtered, and concentrated to dryness under reduced pressure. The residue was purified by flash chromatography on silica gel (dichloromethane:methanol=12:1) to afford the Compound 5 (2.2 mg, yield 3.4%) as a white solid. ¹H NMR (400 MHz, CD₃OD) δ 8.24 (t, J=4.5 Hz, 1H), 7.93 (d, J=8.8 Hz, 2H), 7.72 (d, J=8.7 Hz, 2H), 7.53 (s, 1H), 7.20 (d, J=8.5 Hz, 2H), 7.14 (d, J=8.8 Hz, 2H), 5.03 (s, 1H), 4.59 (t, J=7.7 Hz, 1H), 4.23 (dd, J=47.6, 16.7 Hz, 2H), 3.94 (dt, J=28.5, 8.0 Hz, 2H), 2.49 (s, 1H), 2.30-2.17 (m, 1H), 1.59 (d, J=7.0 Hz, 2H), 1.38 (d, J=57.8 Hz, 1H).LC/MS (ESI) m/z:654 (M+H)⁺.

### Scheme 6: Synthesis of (2S,4R)—N—((R)-1-(4-carbamimidoylthiophen-2-yl)ethyl)-4-(difluoromethoxy)-1-((4-(p-tolyloxy)benzoyl)glycyl)pyrrolidine-2-carboxamide (Compound 6)

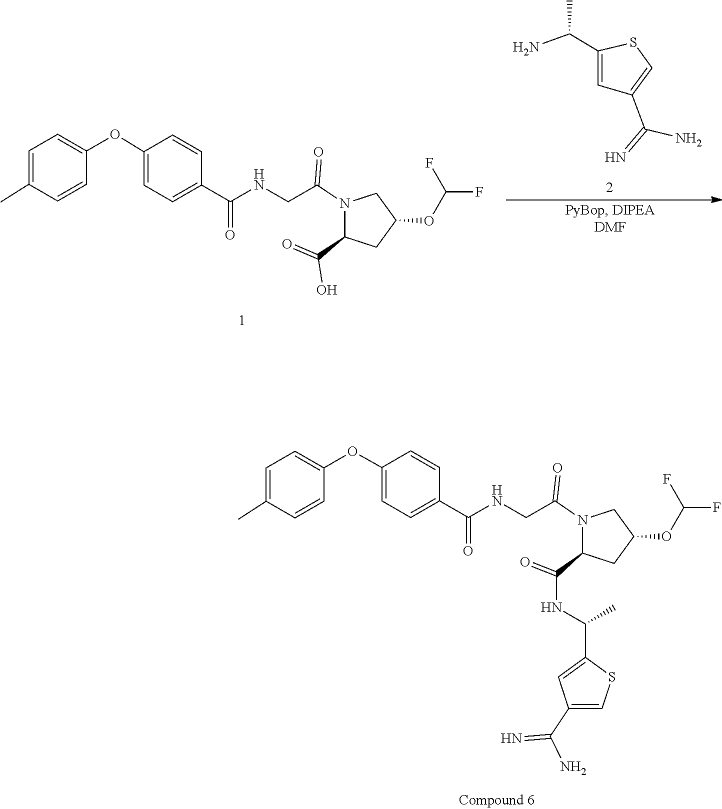 [SMILES: Cc1ccc(Oc2ccc(C(=O)NCC(=O)N3C[C@H](OC(F)F)C[C@H]3C(=O)O)cc2)cc1 ; C[C@@H](N)c1cc(C(=N)N)cs1 ; Cc1ccc(Oc2ccc(C(=O)NCC(=O)N3C[C@H](OC(F)F)C[C@H]3C(=O)N[C@H](C)c3cc(C(=N)N)cs3)cc2)cc1]

Compound 6 was prepared as a white solid from (2S,4R)-4-(difluoromethoxy)-1-((4-(p-tolyloxy)benzoyl)glycyl)pyrrolidine-2-carboxylic acid and (R)-5-(1-aminoethyl)thiophene-3-carboximidamide based on the procedures set forth in Scheme 5 above. ¹H NMR (400 MHz, CD₃OD) δ 8.51 (s, 1H), 8.22 (t, J=3.8 Hz, 1H), 7.82 (t, J=8.8 Hz, 2H), 7.54 (d, J=11.1 Hz, 1H), 7.22 (d, J=8.1 Hz, 2H), 6.98-6.93 (m, 4H), 6.51 (t, J=74.4 Hz, 1H), 5.26 (d, J=7.3 Hz, 1H), 5.01 (s, 1H), 4.57 (s, 1H), 4.19 (dd, J=46.9, 16.8 Hz, 2H), 3.95-3.85 (m, 2H), 2.47 (s, 1H), 2.35 (s, 3H), 2.24 (d, J=12.6 Hz, 1H), 1.61 (dd, J=29.5, 6.9 Hz, 3H). LC/MS (ESI) m/z: 600 (M+H)⁺.

### Scheme 7: (1S,3S,5S)—N—((R)-1-(4-carbamimidoylthiophen-2-yl)ethyl)-5-methyl-2-((4-(p-tolyloxy)benzoyl)glycyl)-2-azabicyclo[3.1.0]hexane-3-carboxamide (Compound 7)

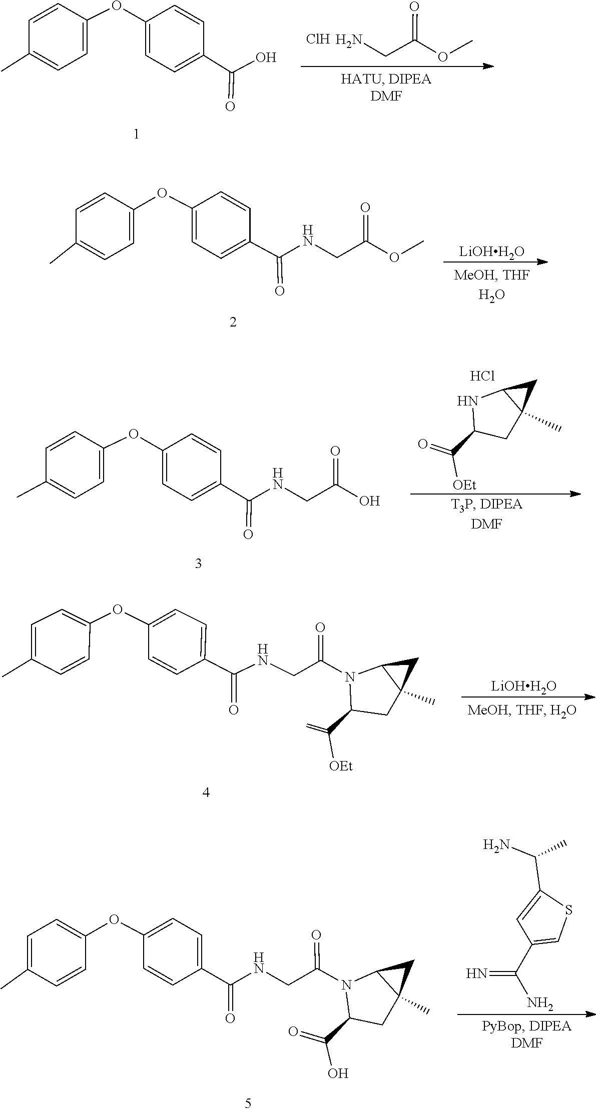

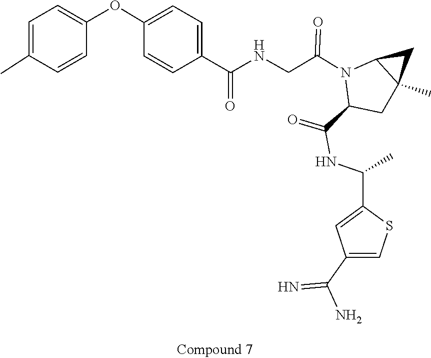

### Step 1: methyl (4-(p-tolyloxy) benzoyl) glycinate (2)

To a mixture of 4-(p-tolyloxy)benzoic acid (1.0 g, 4.4 mmol) and methyl glycinate hydrochloride (825 mg, 6.6 mmol) in DMF (10 mL) was added DIPEA (3.4 g, 26.3 mmol) and HATU (2.5 g, 6.57 mmol) at 0° C. under N₂ atmosphere and the reaction mixture was stirred at room temperature for 1 hour. The mixture was diluted with water and extracted with ethyl acetate twice. The combined organic layers were washed with brine, dried over anhydrous Na₂SO₄, filtered, and concentrated to dryness under reduced pressure. The residue was purified by flash chromatography on silica gel (petroleum ether:ethyl acetate=3:1) to afford methyl (4-(p-tolyloxy)benzoyl)glycinate (1.2 g, yield 92.5%) as a white solid. LC/MS (ESI) m/z: 300 (M+H)⁺.

### Step 2: (4-(p-tolyloxy)benzoyl)glycine (3)

To a mixture of methyl (4-(p-tolyloxy)benzoyl)glycinate (1.2 g, 4.05 mmol) in CH₃OH (7 mL), THF (2.5 mL) and water (2.5 mL) was added LiOH·H₂O (510 mg, 12.15 mmol) at 0° C. and the mixture was stirred under N₂ atmosphere at room temperature for 1 hour. The mixture was acidified with 1 N aq·HCl solution to pH-3 and extracted with ethyl acetate twice. The combined organic layers were washed with brine, dried over anhydrous Na₂SO₄, filtered, and concentrated to dryness under reduced pressure to afford (4-(p-tolyloxy)benzoyl)glycine (890 mg, yield 77.0%) as a white solid, which was used directly in next step without further purification. LC/MS (ESI) (m/z): 286 (M+H)⁺.

### Step 3: ethyl (1S,3S,5S)-5-methyl-2-((4-(p-tolyloxy)benzoyl)glycyl)-2-azabicyclo[3.1.0]hexane-3-carboxylate (4)

To a mixture of (4-(p-tolyloxy)benzoyl)glycine (135 mg, 0.47 mmol) and ethyl (1 S,3S,5S)-5-methyl-2-azabicyclo[3.1.0]hexane-3-carboxylate (120 mg, 0.71 mmol) in DMF (1.5 mL) was added DIPEA (367 mg, 2.84 mmol) and T₃P (450 mg, 1.42 mmol, 50% wt. in ethyl acetate) at 0° C. under N₂ atmosphere and the reaction mixture was stirred at room temperature for 1 hour. The mixture was diluted with water and extracted with ethyl acetate twice. The combined organic layers were washed with brine, dried over anhydrous Na₂SO₄, filtered, and concentrated to dryness under reduced pressure. The residue was purified by flash chromatography on silica gel (petroleum ether:ethyl acetate=1:1) to afford ethyl (1S,3S,5S)-5-methyl-2-((4-(p-tolyloxy)benzoyl)glycyl)-2-azabicyclo[3.1.0]hexane-3-carboxylate (140 mg, yield 67.8%) as a white solid. LC/MS (ESI) m/z:437 (M+H)⁺.

### Step 4: (1S,3S,5S)-5-methyl-2-((4-(p-tolyloxy)benzoyl)glycyl)-2-azabicyclo[3.1.0]hexane-3-carboxylic acid (5)

To a mixture of ethyl (1S,3S,5S)-5-methyl-2-((4-(p-tolyloxy)benzoyl)glycyl)-2-azabicyclo[3.1.0]hexane-3-carboxylate (65 mg, 0.15 mmol) in CH₃OH (0.6 mL), THF (0.2 mL) and water (0.2 mL) was added LiOH·H₂O (19 mg, 0.45 mmol) at 0° C. and the mixture was stirred at room temperature for 1 hour. The mixture was acidified with 1 N aq·HCl solution to pH-3 and extracted with ethyl acetate twice. The combined organic layers were washed with brine, dried over anhydrous Na₂SO₄, filtered, and concentrated to dryness under reduced pressure to afford (1S,3S,5S)-5-methyl-2-((4-(p-tolyloxy)benzoyl)glycyl)-2-azabicyclo[3.1.0]hexane-3-carboxylic acid (50 mg, 81.6% yield) as a white solid, which was used directly in next step without further purification. LC/MS (ESI) (m/z): 409 (M+H)⁺.

### Step 5: (1S,3S,5S)—N—((R)-1-(4-carbamimidoylthiophen-2-yl)ethyl)-5-methyl-2-((4-(p-tolyloxy)benzoyl)glycyl)-2-azabicyclo[3.1.0]hexane-3-carboxamide (Compound 7)

To a mixture of (1 S,3S,5S)-5-methyl-2-((4-(p-tolyloxy)benzoyl)glycyl)-2-azabicyclo[3.1.0]hexane-3-carboxylic acid (36 mg, 0.089 mmol) and (R)-5-(1-aminoethyl)thiophene-3-carboximidamide (15 mg, 0.089 mmol) in DMF (1 mL) was added DIPEA (68 mg, 0.53 mmol) and PyBop (68 mg, 0.13 mmol) at 0° C. under N₂ atmosphere and the reaction mixture was stirred at room temperature for 1 hour. The mixture was diluted with water and extracted with ethyl acetate twice. The combined organic layers were washed with brine, dried over anhydrous Na₂SO₄, filtered, and concentrated to dryness under reduced pressure. The residue was purified by flash chromatography on silica gel (dichloromethane:methanol=12:1) to afford Compound 7 (3.6 mg, yield 7.2%) as a white solid. ¹H NMR (400 MHz, CD₃OD) δ 8.54 (s, 1H), 8.22 (t, J=3.9 Hz, 1H), 7.82 (d, J=8.9 Hz, 2H), 7.50 (d, J=1.3 Hz, 1H), 7.23 (d, J=8.1 Hz, 2H), 6.98-6.94 (m, 4H), 5.22 (q, J=7.0 Hz, 1H), 4.82 (d, J=3.4 Hz, 1H), 4.39 (d, J=16.5 Hz, 1H), 4.25 (d, J=16.5 Hz, 1H), 3.39 (dd, J=6.0, 2.4 Hz, 1H), 2.43 (t, J=12.3 Hz, 1H), 2.35 (s, 3H), 2.18-2.13 (m, 1H), 1.58 (d, J=7.0 Hz, 3H), 1.30 (s, 3H), 1.19 (dd, J=5.6, 2.4 Hz, 1H), 0.77 (t, J=5.1 Hz, 1H). LC/MS (ESI) (m/z): 560 (M+H)⁺.

### Scheme 8: Synthesis of (2S,4R)—N—((R)-1-(4-carbamimidoylthiophen-2-yl)ethyl)-4-(difluoromethoxy)-1-((4-(4-fluorophenoxy)benzoyl)glycyl)pyrrolidine-2-carboxamide (Compound 8)

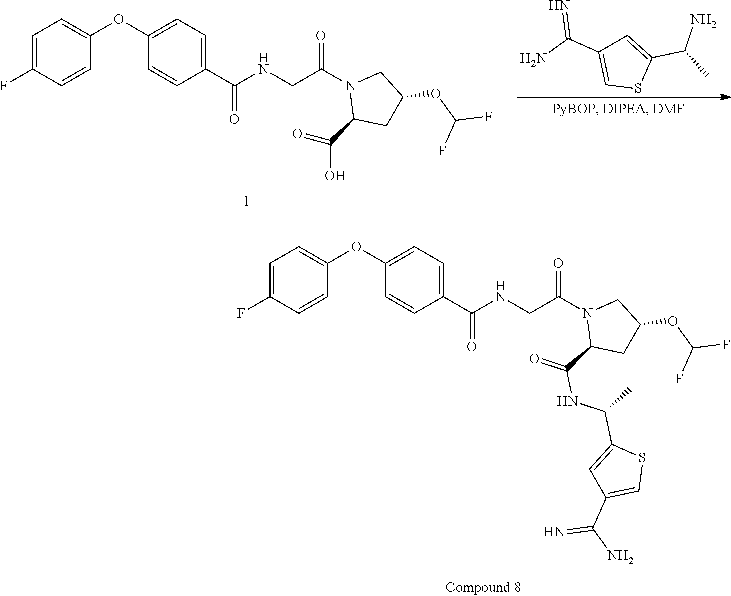 [SMILES: O=C(NCC(=O)N1C[C@H](OC(F)F)C[C@H]1C(=O)O)c1ccc(Oc2ccc(F)cc2)cc1 ; C[C@@H](N)c1cc(C(=N)N)cs1 ; C[C@@H](NC(=O)[C@@H]1C[C@@H](OC(F)F)CN1C(=O)CNC(=O)c1ccc(Oc2ccc(F)cc2)cc1)c1cc(C(=N)N)cs1]

Compound 8 (5 mg, yield 7.5%) was prepared as a white solid from (2S,4R)-4-(difluoromethoxy)-1-((4-(4-fluorophenoxy)benzoyl)glycyl) pyrrolidine-2-carboxylic acid (50 mg, 0.11 mmol) and (R)-5-(1-aminoethyl)thiophene-3-carboximidamide (28 mg, 0.16 mmol) based on the procedures set forth in Scheme 5 above. ¹H NMR (400 MHz, CD₃OD) δ 8.54 (s, 1H), 8.23 (t, J=3.9 Hz, 1H), 7.88-7.83 (m, 2H), 7.54 (d, J=11.4 Hz, 1H), 7.19-7.08 (m, 4H), 7.00 (d, J=8.8 Hz, 2H), 6.50 (td, J=74.5, 15.9 Hz, 1H), 5.38-5.22 (m, 1H), 5.02 (s, 1H), 4.58 (t, J=7.8 Hz, 1H), 4.20 (dd, J=47.0, 16.7 Hz, 2H), 3.93 (dt, J=28.6, 7.0 Hz, 2H), 2.53-2.39 (m, 1H), 2.29-2.14 (m, 1H), 1.62 (dd, J=29.9, 7.0 Hz, 3H). LC/MS (ESI) m/z: 604 (M+H)⁺.

### Scheme 9: Synthesis of (2S,4R)—N—((R)-1-(4-carbamimidoylthiophen-2-yl)ethyl)-1-((9,9-difluoro-9H-fluorene-3-carbonyl)glycyl)-4-(difluoromethoxy)pyrrolidine-2-carboxamide (Compound 9)

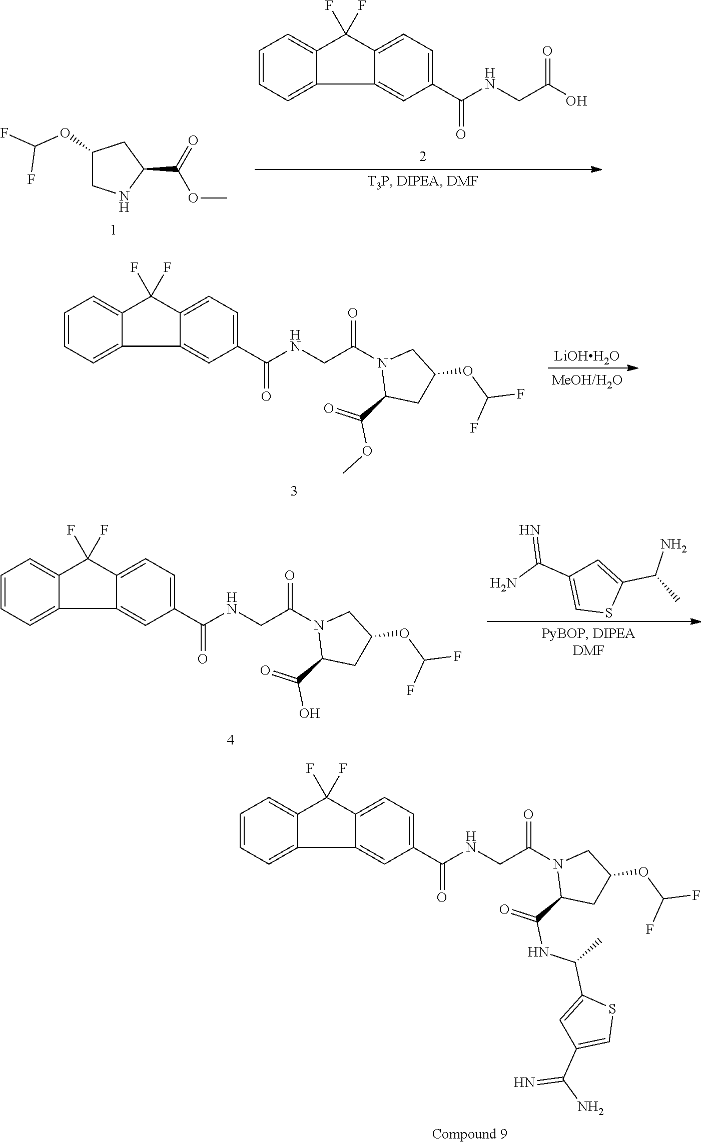

Compound 9 was prepared as a white solid from methyl (2S,4R)-4-(difluoromethoxy)pyrrolidine-2-carboxylate, (9,9-difluoro-9H-fluorene-3-carbonyl)glycine, and (R)-5-(1-aminoethyl)thiophene-3-carboximidamide based on the procedures set forth in Scheme 3. ¹H NMR (400 MHz, CD₃OD) δ 8.50 (s, 1H), 8.25-8.15 (m, 2H), 7.87 (dt, J=11.3, 5.6 Hz, 1H), 7.74 (dd, J=17.1, 7.7 Hz, 2H), 7.65 (d, J=7.4 Hz, 1H), 7.60-7.52 (m, 2H), 7.45 (t, J=7.6 Hz, 1H), 6.74-6.26 (m, 1H), 5.30 (dq, J=14.2, 7.1 Hz, 1H), 5.04 (s, 1H), 4.59 (t, J=7.9 Hz, 1H), 4.24 (dt, J=23.7, 11.9 Hz, 2H), 3.97 (dd, J=11.4, 4.6 Hz, 1H), 3.90 (d, J=11.8 Hz, 1H), 2.65-2.45 (m, 1H), 2.32-2.17 (m, 1H), 1.62 (dd, J=32.0, 6.9 Hz, 3H). LC/MS (ESI) m/z: 618 (M+H)⁺.

### Scheme 10: Synthesis of (2S,4R)—N-((4-carbamimidoylthiophen-2-yl)methyl)-4-(difluoromethoxy)-1-((4-(p-tolyloxy)benzoyl)glycyl)pyrrolidine-2-carboxamide (Compound 10)

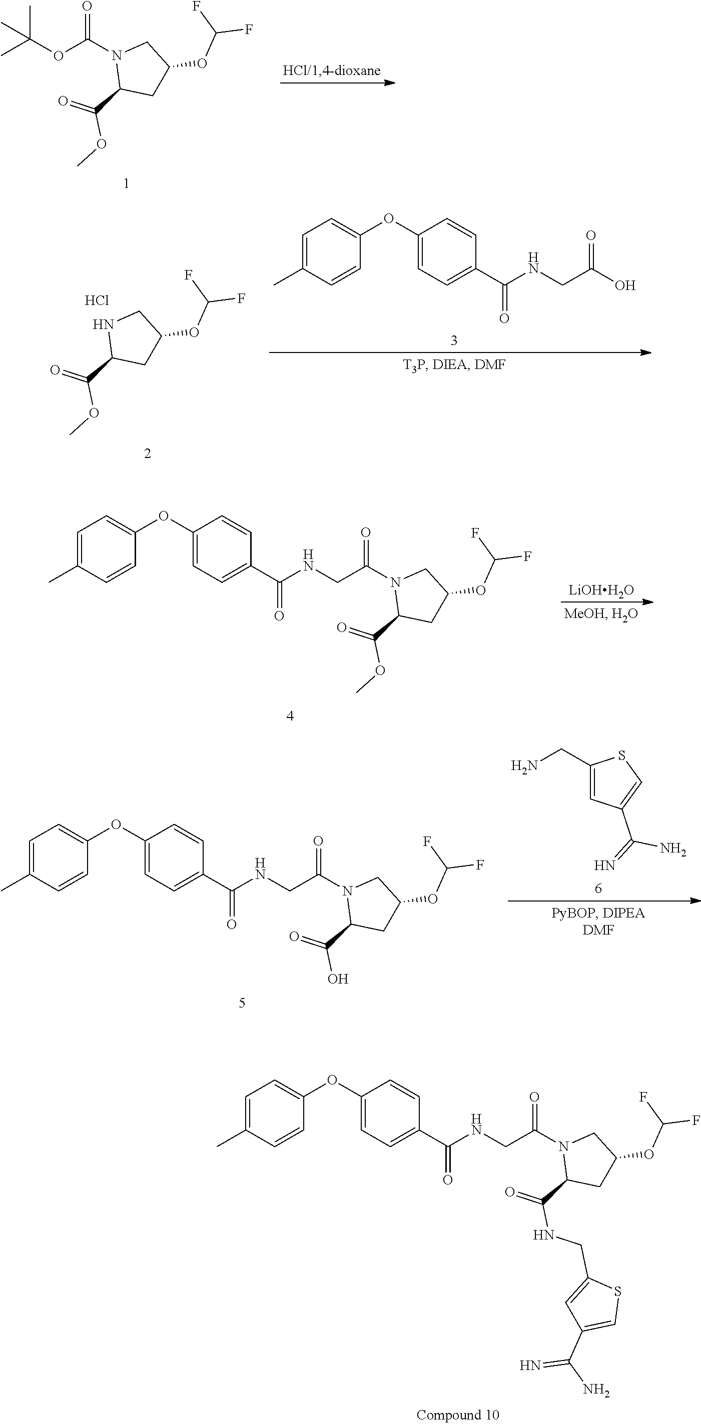

Compound 10 was prepared as a white solid from 1-(tert-butyl) 2-methyl (2S,4R)-4-(difluoromethoxy)pyrrolidine-1,2-dicarboxylate, (4-(p-tolyloxy)benzoyl)glycine, and 5-(aminomethyl)thiophene-3-carboximidamide based on the procedures set forth in Scheme 3. ¹H NMR (400 MHz, CD₃OD) δ 8.53 (s, 1H), 8.22 (t, J=8.4 Hz, 1H), 7.82 (d, J=8.9 Hz, 2H), 7.47 (d, J=35.9 Hz, 1H), 7.22 (d, J=8.2 Hz, 2H), 6.96 (dd, J=14.0, 5.5 Hz, 4H), 6.51 (t, J=74.4 Hz, 1H), 5.00 (s, 1H), 4.66-4.50 (m, 3H), 4.19 (s, 2H), 3.96 (dd, J=11.4, 4.7 Hz, 1H), 3.86 (d, J=11.7 Hz, 1H), 2.46 (s, 1H), 2.35 (s, 3H), 2.26-2.16 (m, 1H). LC/MS (ESI) m/z: 586 (M+H)⁺.

### Scheme 11: Synthesis of (2S,4R)—N-[(1R)-1-(4-carbamimidoylthiophen-2-yl) ethyl]-1-{2-[(9,9-difluorofluoren-3-yl)formamido]acetyl}-4-fluoro-4-(fluoromethyl) pyrrolidine-2-carboxamide (Compound 11)

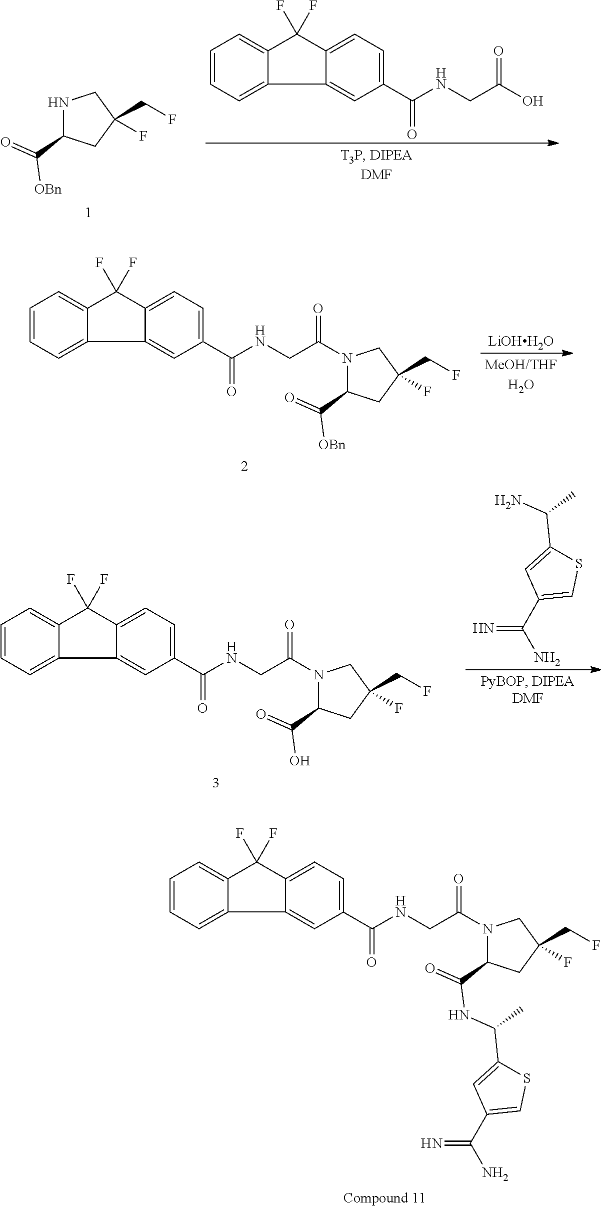

### Step 1: benzyl (2S,4R)-1-{2-[(9,9-difluorofluoren-3-yl)formamido]acetyl}-4-fluoro-4-(fluoromethyl)pyrrolidine-2-carboxylate (2)

To a mixture of benzyl (2S,4R)-4-fluoro-4-(fluoromethyl)pyrrolidine-2-carboxylate (30 mg, 0.12 mmol) and [(9,9-difluorofluoren-3-yl)formamido]acetic acid (36 mg, 0.12 mmol) in DMF (2 mL) was added DIPEA (0.12 mL, 0.7 mmol) and T₃P (224 mg, 0.35 mmol, 50% wt in ethyl acetate) at 0° C. under N₂ atmosphere and the mixture was stirred at 25° C. for 2 hours. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered, and concentrated to dryness under reduced pressure. The residue was purified by flash chromatography on silica gel (PE:ethyl acetate=1:1) to give benzyl (2S,4R)-1-{2-[(9,9-difluorofluoren-3-yl)formamido]acetyl}-4-fluoro-4-(fluoromethyl) pyrrolidine-2-carboxylate (50 mg, yield 78.7%) as a light-yellow solid. LC/MS (ESI) (m/z): 541 (M+H)⁺.

### Step 2: (2S,4R)-1-{2-[(9,9-difluorofluoren-3-yl)formamido]acetyl}-4-fluoro-4-(fluoromethyl)pyrrolidine-2-carboxylic acid (3)

To a solution of benzyl (2S,4R)-1-{2-[(9,9-difluorofluoren-3-yl)formamido]acetyl}-4-fluoro-4-(fluoromethyl)pyrrolidine-2-carboxylate (50 mg, 0.093 mmol) in MeOH (2 mL) and THF (1 mL) was added a solution of LiOH (16 mg, 0.37 mmol) in water (1 mL) at 0° C., and the mixture was stirred at 25° C. for 1 hour. The mixture was diluted with H₂O and washed with ethyl acetate twice. The aqueous layer was acidified with 0.5 M aq·HCl and extracted with ethyl acetate twice. The combined organic layers were washed with brine, dried over anhydrous Na₂SO₄, filtered, and concentrated to dryness under reduced pressure to give (2S,4R)-1-{2-[(9,9-difluorofluoren-3-yl)formamido]acetyl}-4-fluoro-4-(fluoromethyl)pyrrolidine-2-carboxylic acid (32 mg, yield 76.8%) as a light oil, which was used directly in the next step without further purification. LC/MS (ESI) (m/z): 451 (M+H)⁺.

### Step 3: (2S,4R)—N-[(1R)-1-(4-carbamimidoylthiophen-2-yl)ethyl]-1-{2-[(9,9-difluorofluoren-3-yl)formamido]acetyl}-4-fluoro-4-(fluoromethyl)pyrrolidine-2-carboxamide (Compound 11)

To a mixture of (2S,4R)-1-{2-[(9,9-difluorofluoren-3-yl)formamido]acetyl}-4-fluoro-4-(fluoromethyl)pyrrolidine-2-carboxylic acid (30 mg, 0.067 mmol) and 5-[(1R)-1-aminoethyl]thiophene-3-carboximidamide (14 mg, 0.08 mmol) in DMF (2 mL) was added DIPEA (0.058 mL, 0.33 mmol) and PyBop (52 mg, 0.10 mmol) at 0° C. and the mixture was stirred at 25° C. for 0.5 hour. The mixture was washed with water and extracted with ethyl acetate twice. The combined organic layers were washed with brine, dried over anhydrous Na₂SO₄, filtered, and concentrated to dryness under reduced pressure. The residue was purified by prep-TLC (DCM:MeOH=10:1) and prep-HPLC to give Compound 11 (2.5 mg, yield 6.2%) as a white solid. ¹H NMR (400 MHz, CD30D) δ 8.28-8.18 (m, 2H), 7.93-7.85 (m, 1H), 7.82-7.73 (m, 2H), 7.68 (d, J=7.6 Hz, 1H), 7.63-7.55 (m, 2H), 7.47 (t, J=7.4 Hz, 1H), 5.34-5.25 (m, 1H), 4.84-4.76 (m, 2H), 4.75-4.65 (m, 2H), 4.38 (d, J=16.8 Hz, 1H), 4.22-4.12 (m, 2H), 2.79-2.51 (m, 1H), 2.35-2.16 (m, 1H), 1.65 (dd, J=32.4, 32.4 Hz, 3H). LC/MS (ESI) (m/z): 602 (M+H)⁺.

### Scheme 12: Synthesis of (2S,4R)—N-[(1R)-1-(4-carbamimidoylthiophen-2-yl)ethyl]-1-[2-(9H-fluoren-3-ylformamido)acetyl]-4-fluoro-4-(fluoromethyl)pyrrolidine-2-carboxamide (Compound 12)

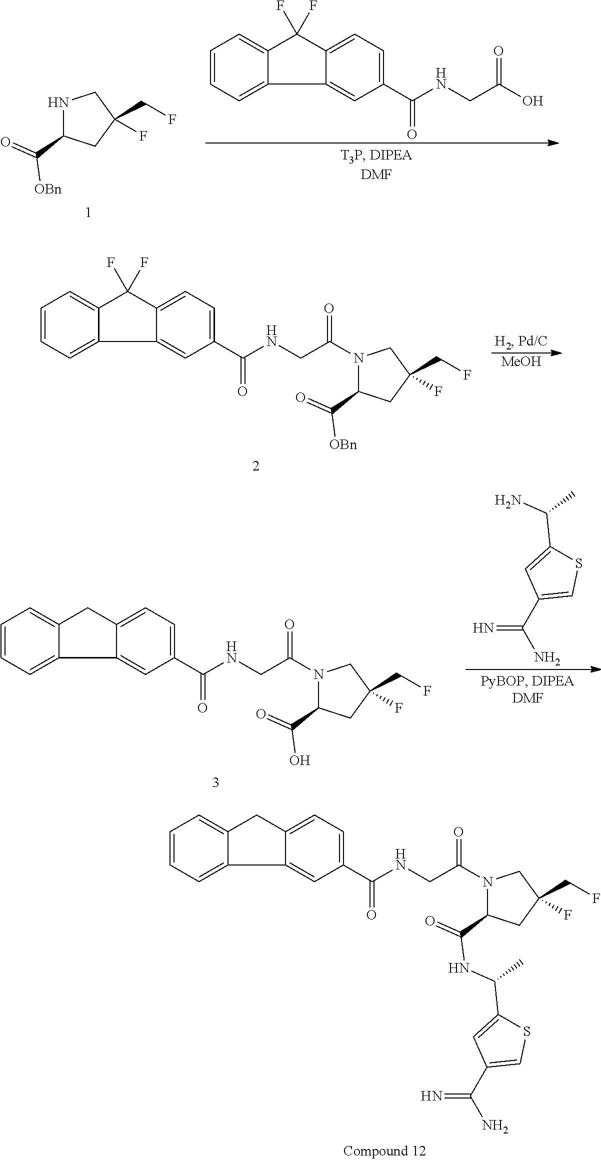

### Step 1: benzyl (2S,4R)-1-{2-[(9,9-difluorofluoren-3-yl)formamido]acetyl}-4-fluoro-4-(fluoromethyl)pyrrolidine-2-carboxylate (2)

To a mixture of benzyl (2S,4R)-4-fluoro-4-(fluoromethyl)pyrrolidine-2-carboxylate (25 mg, 0.098 mmol) and [(9,9-difluorofluoren-3-yl)formamido]acetic acid (30 mg, 0.098 mmol) in DMF (2 mL) was added DIPEA (0.11 mL, 0.6 mmol) and T₃P (187 mg, 0.29 mmol, 50% wt. in ethyl acetate) at 0° C. under N₂ atmosphere and the mixture was stirred at 25° C. for 1 hour. The mixture was diluted with ethyl acetate and washed with saturated aq·NaHCO₃ solution. The aqueous layer was extracted with ethyl acetate twice. The combined organic layers were washed with brine, dried over anhydrous Na₂SO₄, filtered, and concentrated to dryness under reduced pressure. The residue was purified by flash chromatography on silica gel (PE:ethyl acetate=1:1) to give benzyl (2S,4R)-1-{2-[(9,9-difluorofluoren-3-yl)formamido]acetyl}-4-fluoro-4-(fluoromethyl) pyrrolidine-2-carboxylate (50 mg, yield 94.4%) as a light oil. LC/MS (ESI) (m/z): 541 (M+H)⁺.

### Step 2: (2S,4R)-1-[2-(9H-fluoren-3-ylformamido)acetyl]-4-fluoro-4-(fluoromethyl) pyrrolidine-2-carboxylic acid (3)

To a solution of benzyl (2S,4R)-1-{2-[(9,9-difluorofluoren-3-yl)formamido]acetyl}-4-fluoro-4-(fluoromethyl)pyrrolidine-2-carboxylate (50 mg, 0.093 mmol) in MeOH (3 mL) was added Pd/C (10 m, 10% wt) at 0° C., and the mixture was degassed under N₂ atmosphere for three times and stirred under a H₂ balloon at 25° C. for 4 hours. The mixture was filtered, and the filtrate was concentrated to dryness under reduced pressure to give (2S,4R)-1-[2-(9H-fluoren-3-ylformamido)acetyl]-4-fluoro-4-(fluoromethyl)pyrrolidine-2-carboxylic acid (38 mg, yield 99.1%) as a light-yellow oil, which was used directly in the next step without further purification. LC/MS (ESI) (m/z): 415 (M+H)⁺.

### Step 3: (2S,4R)—N-[(1R)-1-(4-carbamimidoylthiophen-2-yl)ethyl]-1-[2-(9H-fluoren-3-ylformamido)acetyl]-4-fluoro-4-(fluoromethyl)pyrrolidine-2-carboxamide (Compound 12)

To a mixture of (2S,4R)-1-[2-(9H-fluoren-3-ylformamido)acetyl]-4-fluoro-4-(fluoromethyl)pyrrolidine-2-carboxylic acid (35 mg, 0.084 mmol) and 5-[(1R)-1-aminoethyl]thiophene-3-carboximidamide (17 mg, 0.10 mmol) in DMF (2 mL) was added DIPEA (0.074 mL, 0.42 mmol) and PyBop (66 mg, 0.13 mmol) at 0° C. and the mixture was stirred at 25° C. for 1 hour. The mixture was washed with water and extracted with ethyl acetate twice. The combined organic layers were washed with brine, dried over anhydrous Na₂SO₄, filtered, and concentrated to dryness under reduced pressure. The residue was purified by prep-TLC (DCM:MeOH=10:1) and prep-HPLC to give Compound 12 (2.3 mg, yield 4.8%) as a white solid. ¹H NMR (400 MHz, CD₃OD) δ 8.35-8.31 (m, 1H), 8.24 (dd, J=27.2, 27.2 Hz, 1H), 7.92-7.80 (m, 2H), 7.67 (t, J=7.4 Hz, 1H), 7.63-7.55 (m, 2H), 7.44-7.35 (m, 2H), 5.43-5.27 (m, 1H), 4.84-4.77 (m, 2H), 4.73-4.66 (m, 2H), 4.37 (d, J=16.4 Hz, 1H), 4.25-4.11 (m, 2H), 3.97 (s, 2H), 2.87-2.58 (m, 1H), 2.39-2.14 (m, 1H), 1.65 (dd, J=33.6, 33.6 Hz, 3H). LC/MS (ESI) (m/z): 566 (M+H)⁺.

### Scheme 13: Synthesis of 2S,4R)—N-[(1R)-1-(4-carbamimidoylthiophen-2-yl)ethyl]-4-fluoro-4-(fluoromethyl)-1-(2-{[4-(4-fluorophenoxy)phenyl]formamido}acetyl) pyrrolidine-2-carboxamide (Compound 13)

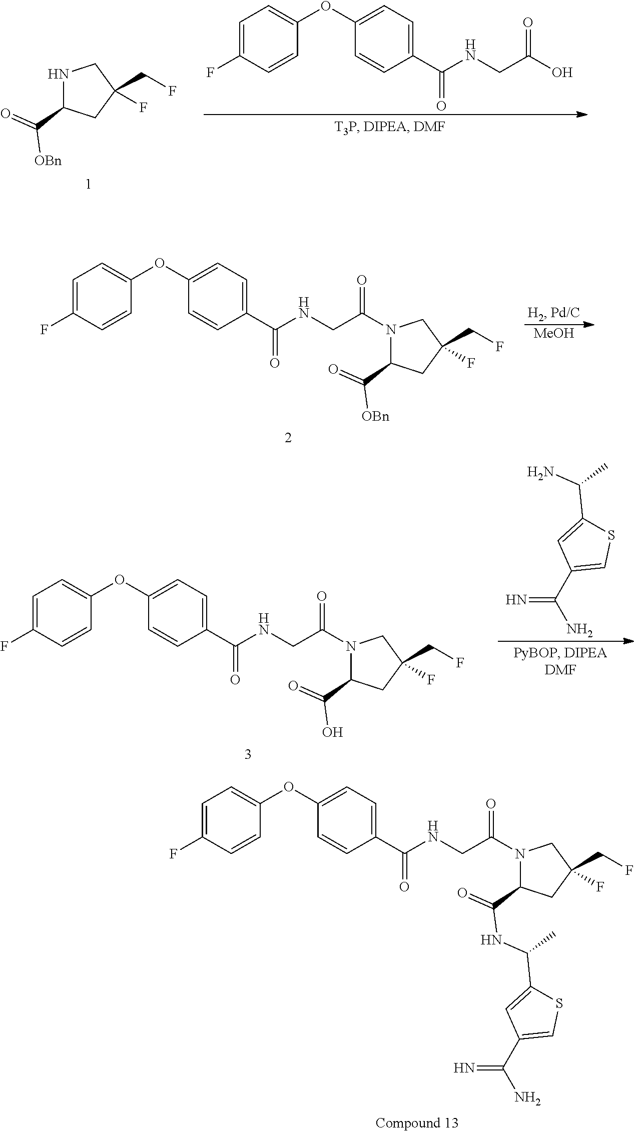

Compound 13 was prepared as a white solid from benzyl (2S,4R)-4-fluoro-4-(fluoromethyl)pyrrolidine-2-carboxylate, {[4-(4-fluorophenoxy)phenyl]formamido}acetic acid, and 5-[(1R)-1-aminoethyl]thiophene-3-carboximidamide based on the procedures set forth in Scheme 12. ¹H NMR (400 MHz, CD₃OD) δ 8.23 (dd, J=9.6, 9.6 Hz, 1H), 7.88-7.82 (m, 2H), 7.58-7.53 (m, 1H), 7.19-7.13 (m, 2H), 7.12-7.07 (m, 2H), 7.02-6.98 (m, 2H), 5.44-5.21 (m, 1H), 4.79-4.74 (m, 1H), 4.69-4.61 (m, 2H), 4.29 (d, J=16.8 Hz, 1H), 4.17-4.08 (m, 2H), 4.00-3.87 (m, 1H), 2.70-2.53 (m, 1H), 2.35-2.12 (m, 1H), 1.60 (dd, J=29.6, 29.6 Hz, 3H). LC/MS (ESI) (m/z): 588 (M+H)⁺.

### Scheme 14: Synthesis of (2S,4R)—N—((R)-1-(4-carbamimidoylthiophen-2-yl)ethyl)-4-fluoro-4-(fluoromethyl)-1-((4-(p-tolyloxy)benzoyl)glycyl)pyrrolidine-2-carboxamide (Compound 14)

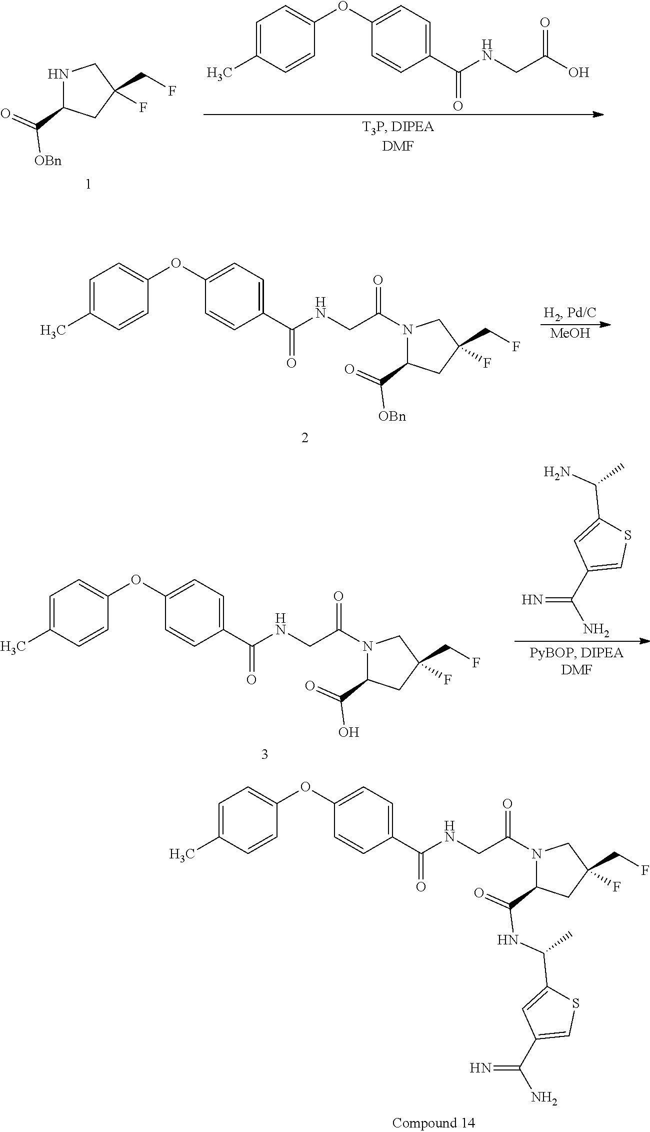

Compound 14 was prepared as a white solid from benzyl (2S,4R)-4-fluoro-4-(fluoromethyl)pyrrolidine-2-carboxylate, {[4-(4-methylphenoxy)phenyl]formamido}acetic acid, and 5-[(1R)-1-aminoethyl]thiophene-3-carboximidamide based on the procedures set forth in Scheme 12. ¹H NMR (400 MHz, CD₃OD) δ 8.26-8.20 (m, 1H), 7.86-7.80 (m, 2H), 7.58-7.52 (m, 1H), 7.23 (d, J=8.4 Hz, 2H), 6.99-6.93 (m, 4H), 5.40-5.19 (m, 1H), 4.78-4.72 (m, 1H), 4.68-4.60 (m, 2H), 4.29 (d, J=16.4 Hz, 1H), 4.17-4.07 (m, 2H), 4.00-3.87 (m, 1H), 2.79-2.54 (m, 1H), 2.35 (s, 3H), 2.29-2.11 (m, 1H), 1.62 (dd, J=29.6, 29.6 Hz, 3H). LC/MS (ESI) (m/z): 584 (M+H)⁺.

### Scheme 15: Synthesis of (2S,4R)—N-[(1R)-1-(4-carbamimidoylthiophen-2-yl)ethyl]-4-fluoro-4-(fluoromethyl)-1-[2-({4-[4-(trifluoromethyl)phenoxy]phenyl}formamido) acetyl]pyrrolidine-2-carboxamide (Compound 15)

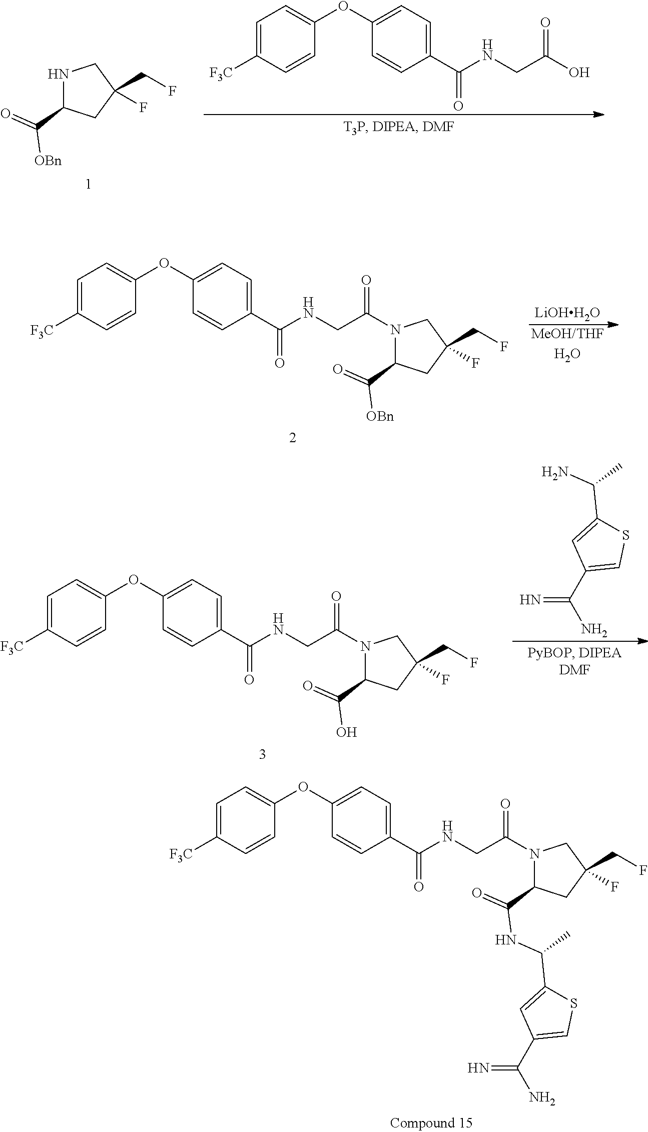

Compound 15 was prepared as a white solid from benzyl (2S,4R)-4-fluoro-4-(fluoromethyl)pyrrolidine-2-carboxylate, (4-(4-(trifluoromethyl)phenoxy)benzoyl)glycine, and 5-[(1R)-1-aminoethyl]thiophene-3-carboximidamide based on the procedures set forth in Scheme 12. ¹H NMR (400 MHz, CD₃OD) δ 8.24 (dd, J=10.8, 10.8 Hz, 1H), 7.96-7.89 (m, 2H), 7.71 (d, J=8.8 Hz, 2H), 7.56 (d, J=10.4 Hz, 1H), 7.19 (d, J=8.4 Hz, 2H), 7.15-7.11 (m, 2H), 5.37-5.21 (m, 1H), 4.80-4.74 (m, 1H), 4.70-4.61 (m, 2H), 4.28 (d, J=16.4 Hz, 1H), 4.19-4.08 (m, 2H), 4.02-3.88 (m, 1H), 2.80-2.50 (m, 1H), 2.40-2.13 (m, 1H), 1.62 (dd, J=30.4, 30.4 Hz, 3H). LC/MS (ESI) (m/z): 638 (M+H)⁺.

### Scheme 16: Synthesis of (1S,3S,5S)—N-[(1R)-1-(4-carbamimidoylthiophen-2-yl)ethyl]-2-(2-{[4-(4-fluorophenoxy)phenyl]formamido}acetyl)-5-methyl-2-azabicyclo[3.1.0]hexane-3-carboxamide (Compound 16)

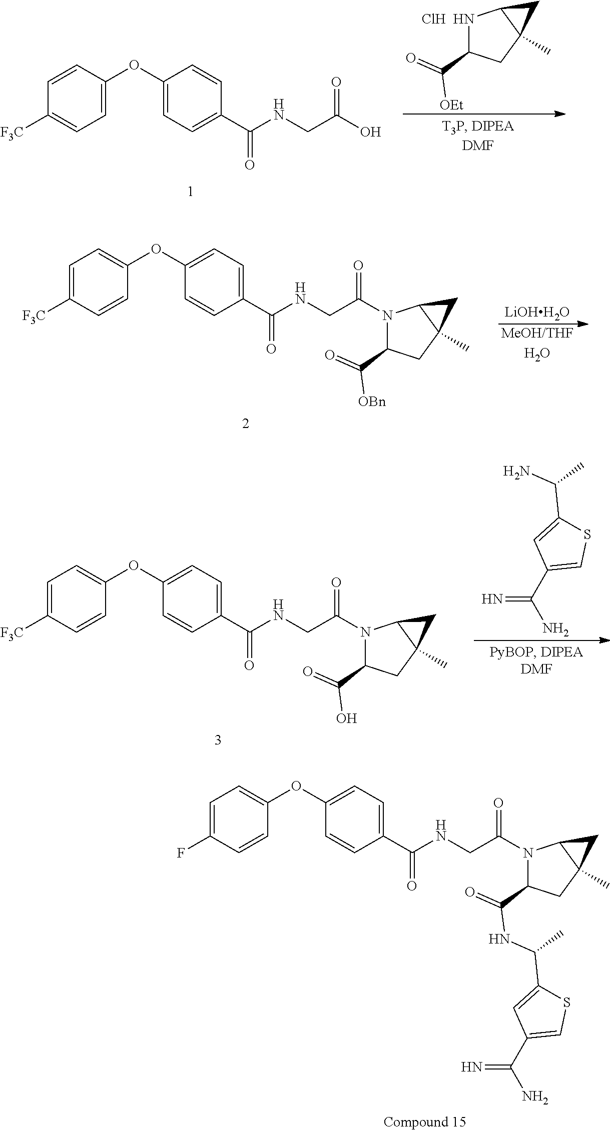

Compound 16 was prepared as a white solid from {[4-(4-fluorophenoxy)phenyl]formamido}acetic acid, ethyl (1 S,3S,5S)-5-methyl-2-azabicyclo[3.1.0]hexane-3-carboxylate hydrochloride, and 5-[(1R)-1-aminoethyl]thiophene-3-carboximidamide based on the procedures set forth in Scheme 7. ¹H NMR (400 MHz, CD₃OD) δ 8.24-8.18 (m, 1H), 7.87-7.81 (m, 2H), 7.52-7.48 (m, 1H), 7.19-7.12 (m, 2H), 7.11-7.06 (m, 2H), 7.01-6.96 (m, 2H), 5.25-5.18 (m, 1H), 4.81-4.80 (m, 1H), 4.32 (dd, J=55.6, 55.6 Hz, 2H), 3.41-3.35 (m, 1H), 2.43 (t, J=12.4 Hz, 1H), 2.15 (dd, J=13.2, 13.2 Hz, 1H), 1.59 (dd, J=23.2, 23.2 Hz, 3H), 1.29 (s, 3H), 1.18 (dd, J=5.6, 5.6 Hz, 1H), 0.77 (t, J=5.4 Hz, 1H). LC/MS (ESI) (m/z): 564 (M+H)⁺.

### Scheme 17: Synthesis of (2S,4R)—N—((R)-1-(4-carbamimidoylthiophen-2-yl)ethyl)-4-fluoro-1-((4-(4-fluorophenoxy)benzoyl)glycyl)-4-(methoxymethyl)pyrrolidine-2-carboxamide (Compound 17)

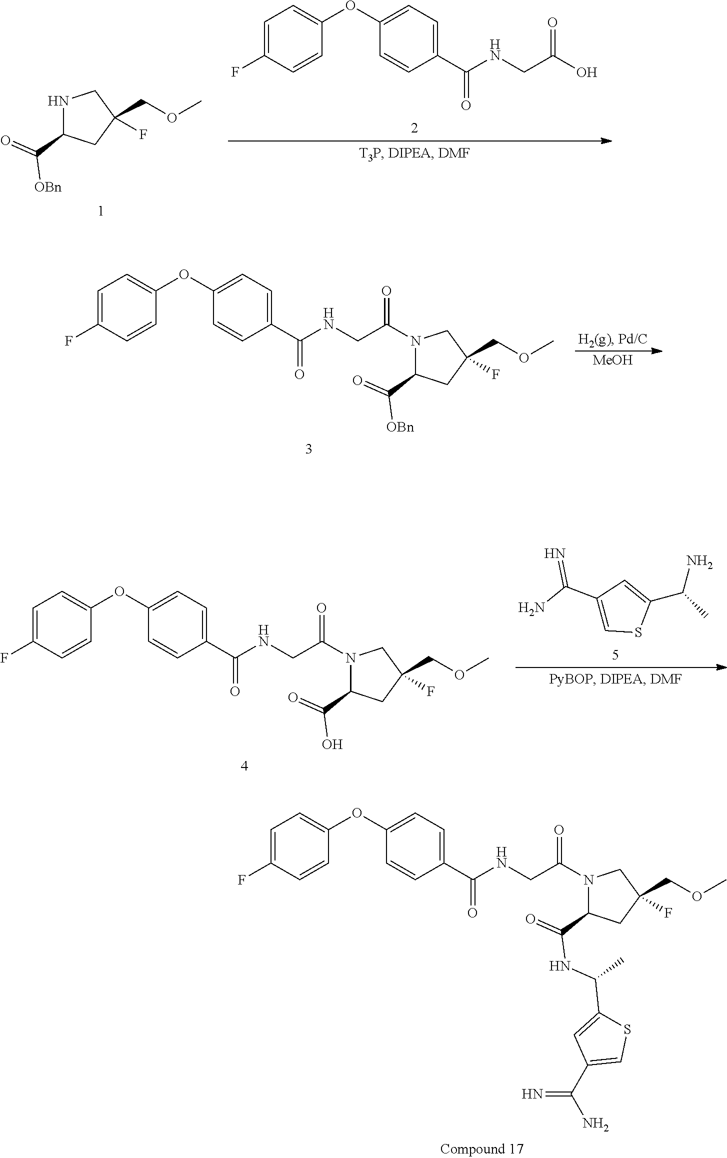

Compound 17 was prepared as a white solid from benzyl (2S,4R)-4-fluoro-4-(methoxymethyl)pyrrolidine-2-carboxylate, 4-(4-fluorophenoxy)benzoyl)glycine, and 5-[(1R)-1-aminoethyl]thiophene-3-carboximidamide based on the procedures set forth in Scheme 12. ¹H NMR (400 MHz, CD₃OD) δ 8.54 (s, 1H), 8.23 (dd, J=9.2, 1.5 Hz, 1H), 7.84 (t, J=8.2 Hz, 2H), 7.55 (dd, J=5.2, 3.9 Hz, 1H), 7.19-7.13 (m, 2H), 7.11-7.07 (m, 2H), 7.01-6.97 (m, 2H), 5.26 (q, J=6.7 Hz, 1H), 4.69-4.56 (m, 1H), 4.28 (d, J=16.7 Hz, 1H), 4.14-4.00 (m, 2H), 3.89 (dd, J=33.5, 12.2 Hz, 1H), 3.73-3.64 (m, 2H), 3.43 (d, J=4.8 Hz, 3H), 2.69-2.47 (m, 1H), 2.18 (ddd, J=36.8, 14.2, 9.6 Hz, 1H), 1.60 (t, J=15.1 Hz, 3H). LC/MS (ESI) m/z: 600 (M+H)⁺.

### Scheme 18: Synthesis of (2S,4R)—N—((R)-1-(4-carbamimidoylthiophen-2-yl)ethyl)-4-fluoro-4-(methoxymethyl)-1-((4-(4-(trifluoromethyl)phenoxy)benzoyl)glycyl)pyrrolidine-2-carboxamide (Compound 18)

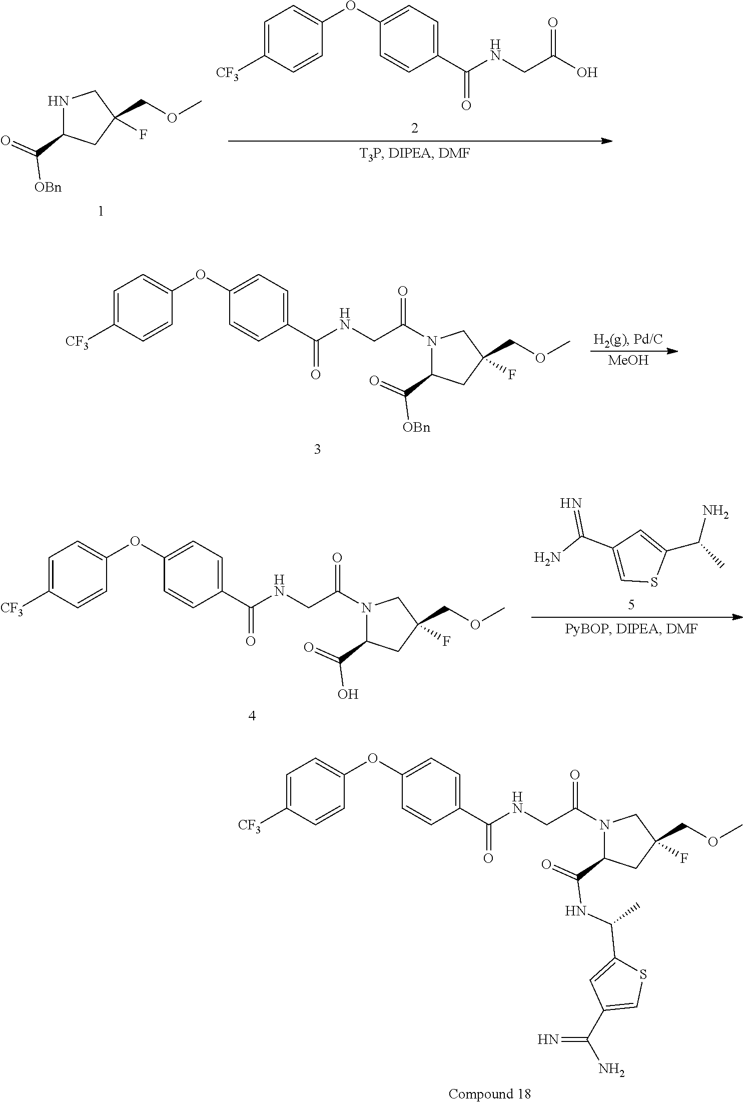

Compound 18 was prepared as a white solid from benzyl (2S,4R)-4-fluoro-4-(methoxymethyl)pyrrolidine-2-carboxylate, (4-(4-(trifluoromethyl)phenoxy)benzoyl)glycine, and 5-[(1R)-1-aminoethyl]thiophene-3-carboximidamide based on the procedures set forth in Scheme 12. ¹H NMR (400 MHz, CD₃OD) δ 8.55 (s, 1H), 8.23 (t, J=5.8 Hz, 1H), 7.92 (d, J=8.7 Hz, 2H), 7.70 (d, J=8.6 Hz, 2H), 7.55 (d, J=8.3 Hz, 1H), 7.19 (d, J=8.6 Hz, 2H), 7.13 (d, J=8.7 Hz, 2H), 5.42-5.20 (m, 1H), 4.65-4.57 (m, 1H), 4.30 (d, J=16.6 Hz, 1H), 4.15-4.00 (m, 2H), 3.89 (dd, J=33.6, 12.2 Hz, 1H), 3.73-3.64 (m, 2H), 3.42 (d, J=5.4 Hz, 3H), 2.68-2.47 (m, 1H), 2.18 (ddd, J=37.0, 14.2, 9.6 Hz, 1H), 1.62 (dd, J=30.6, 7.1 Hz, 3H). LC/MS (ESI) m/z: 650 (M+H)⁺.

### Scheme 19: Synthesis of (2S,4R)—N—((R)-1-(4-carbamimidoylthiophen-2-yl) ethyl)-4-fluoro-4-(methoxymethyl)-1-((4-(p-tolyloxy)benzoyl)glycyl)pyrrolidine-2-carboxamide (Compound 19)

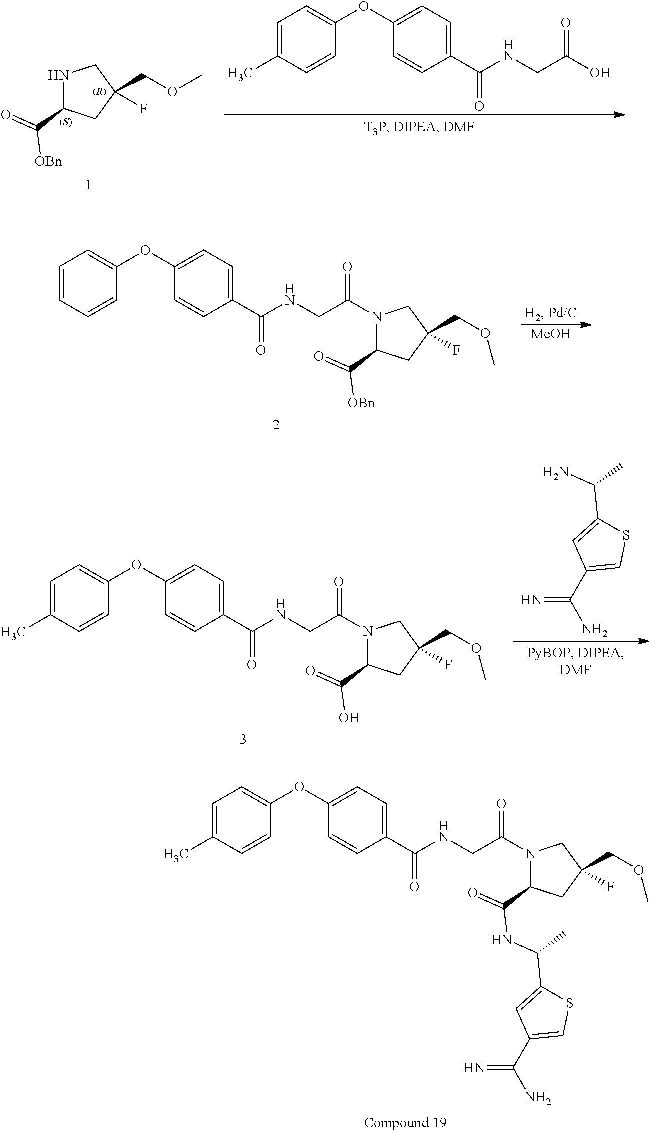

Compound 19 was prepared as a white solid from benzyl (2S,4R)-4-fluoro-4-(methoxymethyl)pyrrolidine-2-carboxylate, {[4-(4-methylphenoxy)phenyl]formamido}acetic acid, and 5-[(1R)-1-aminoethyl]thiophene-3-carboximidamide based on the procedures set forth in Scheme 12. ¹H NMR (400 MHz, CD₃OD) δ 8.21 (d, J=1.6 Hz, 1H), 7.84-7.79 (m, 2H), 7.57-7.51 (m, 1H), 7.22 (d, J=8.4 Hz, 2H), 6.98-6.93 (m, 4H), 5.38-5.21 (m, 1H), 4.63-4.57 (m, 2H), 4.27 (d, J=16.8 Hz, 1H), 4.12-3.99 (m, 2H), 3.72-3.63 (m, 2H), 3.42 (d, J=4.8 Hz, 3H), 2.59-2.47 (m, 1H), 2.35 (s, 3H), 2.25-2.10 (m, 1H), 1.61 (dd, J=29.9, 7.0 Hz, 3H). LC/MS (ESI) m/z: 596 (M+H)⁺.

### Scheme 20: (2S,4R)—N—((R)-1-(4-carbamimidoylthiophen-2-yl)ethyl)-1-((9,9-difluoro-9H-fluorene-3-carbonyl)glycyl)-4-fluoro-4-(methoxymethyl)pyrrolidine-2-carboxamide (Compound 20)

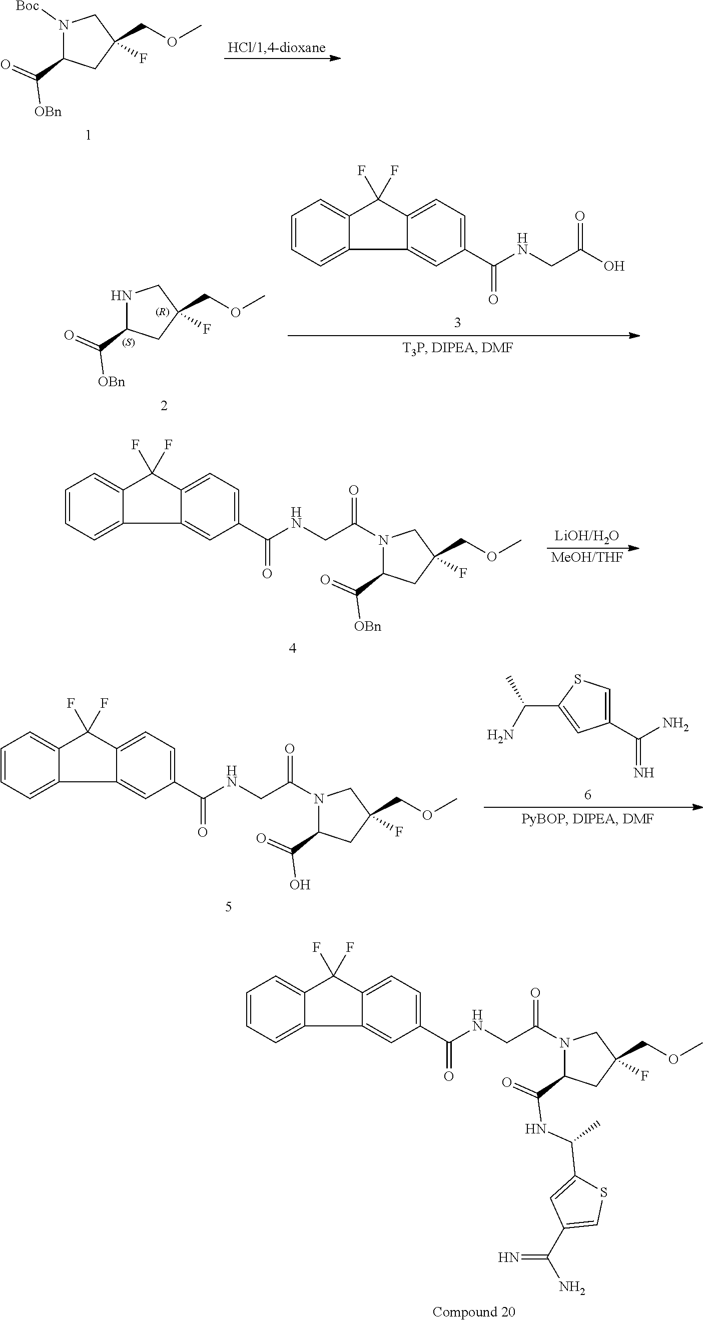

Compound 20 was prepared as a white solid from 2-benzyl-1-(tert-butyl)-(2S,4R)-4-fluoro-4-(methoxymethyl)pyrrolidine-1,2-dicarboxylate, (9,9-difluoro-9H-fluorene-3-carbonyl)glycine, and (R)-5-(1-aminoethyl)thiophene-3-carboximidamide based on the procedures set forth in Scheme 3. ¹H NMR (400 MHz, CD₃OD) δ 8.26-8.16 (m, 2H), 7.89 (dd, J=7.8, 1.5 Hz, 1H), 7.77 (d, J=7.6 Hz, 1H), 7.73 (d, J=7.7 Hz, 1H), 7.66 (d, J=7.5 Hz, 1H), 7.61-7.54 (m, 2H), 7.46 (t, J=7.5 Hz, 1H), 5.34-5.24 (m, 1H), 4.66-4.60 (m, 1H), 4.36 (d, J=16.7 Hz, 1H), 4.17 (d, J=16.8 Hz, 1H), 4.07 (dd, J=19.3, 12.5 Hz, 1H), 3.98-3.86 (m, 1H), 3.75 (t, J=7.6 Hz, 1H), 3.70 (d, J=6.8 Hz, 1H), 3.44 (d, J=6.1 Hz, 3H), 2.62-2.50 (m, 1H), 2.23-2.14 (m, 1H), 1.59 (d, J=7.0 Hz, 3H). LC/MS (ESI) (m/z): 614 (M+H)⁺.

### Scheme 21: Synthesis of (S)—N—((R)-1-(4-carbamimidoylthiophen-2-yl)ethyl)-7-((9,9-difluoro-9H-fluorene-3-carbonyl)glycyl)-1,4-dioxa-7-azaspiro[4.4]nonane-8-carboxamide (Compound 21)

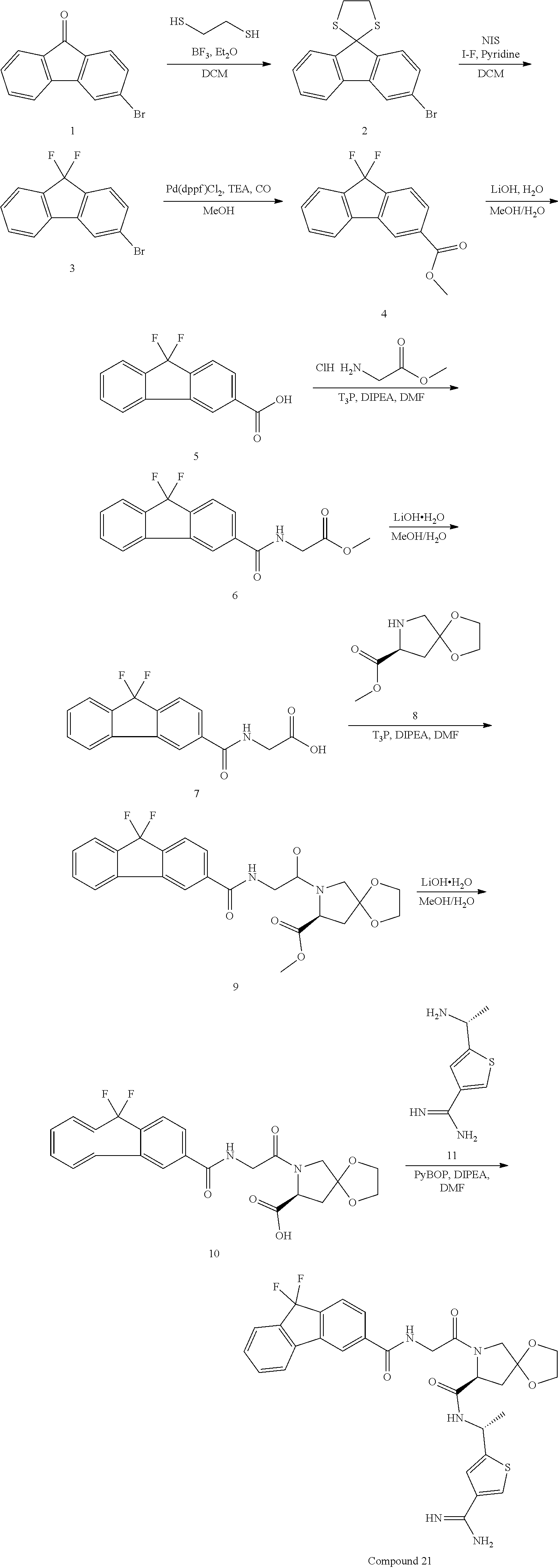

### Step 1: 3-bromospiro[fluorene-9,2′-[1,3]dithiolane](2)

A solution of 3-bromo-9H-fluoren-9-one (1.95 g, 7.53 mmol) in dichloromethane (25 mL) was added ethane-1,2-dithiol (3.56 g, 37.8 mmol) and BF₃·Et₂O (8 mL) at 0° C. The mixture was stirred at room temperature under N₂ atmosphere for 4 hours. The mixture was diluted with water and extracted with dichloromethane (2×50 mL). The combined organic layers were washed with brine, dried over anhydrous Na₂SO₄, filtered, and concentrated to dryness under reduced pressure. The residue was purified by flash chromatography on silica gel (petroleum ether:ethyl acetate=50:1) to give 3-bromospiro[fluorene-9,2′-[1,3]dithiolane](2.3 g, yield 91.1%) as a white solid.

### Step 2: 3-bromo-9,9-difluoro-9H-fluorene (3)

A solution of N-iodosuccinimide (10.3 g, 45.93 mmol) in dichloromethane (15 mL) was added dropwise tp HF-pyridine (25 mL) at 0° C. and the mixture was stirred for 10 minutes. To the above reaction mixture, a solution of 3-bromospiro[fluorene-9,2′-[1,3]dithiolane](2.2 g, 6.56 mmol) in dichloromethane (15 mL) was added and the mixture was stirred at room temperature for 2 hours. The reaction mixture was quenched with water and extracted with dichloromethane (2×30 mL). The combined organic layers were washed with brine, dried over anhydrous Na₂SO₄, filtered, and concentrated to dryness under reduced pressure. The residue was purified by flash column chromatography on silica gel (petroleum ether:ethyl acetate=50:1) to give 3-bromo-9,9-difluoro-9H-fluorene (1.78 g, yield 96.5%) as a white solid.

### Step 3: methyl 9,9-difluoro-9H-fluorene-3-carboxylate (4)

A solution of 3-bromo-9,9-difluoro-9H-fluorene (780 mg, 2.77 mmol) in methanol (20 mL) was added Pd(dppf)Cl₂ (402 mg, 0.55 mmol) and triethylamine (840 mg, 8.31 mmol). The mixture was stirred at 50° C. under CO atmosphere overnight. The reaction mixture was quenched with saturated aq·NH₄Cl solution. The resulting mixture was extracted with ethyl acetate (3×50 mL). The combined organic layers were washed with water (2×50 mL) and brine (2×50 mL), dried over anhydrous Na₂SO₄, filtered, and concentrated to dryness under reduced pressure. The residue was purified by flash column chromatography on silica gel (petroleum ether:ethyl acetate=20:1) to give methyl 9,9-difluoro-9H-fluorene-3-carboxylate (605 mg, yield 83.9%) as a white solid.

### Step 4: 9,9-difluoro-9H-fluorene-3-carboxylic acid (5)

A solution of methyl 9,9-difluoro-9H-fluorene-3-carboxylate (600 mg, 2.31 mmol) in tetrahydrofuran (6 mL) and water (2 mL) was added a solution of LiOH·H₂O (291 mg, 6.92 mmol) at 0° C. and the mixture was stirred at room temperature for 16 hours. The mixture was acidified with 1 N aq·HCl solution to pH<3 and extracted with ethyl acetate (2×50 mL). The combined organic layers were concentrated to dryness under reduced pressure to give 9,9-difluoro-9H-fluorene-3-carboxylic acid (550 mg, yield 96.7%) as a white solid, which was used directly in next step without further purification. LC/MS (ESI) (m/z): 247(M+H)⁺.

### Step 5: methyl (9,9-difluoro-9H-fluorene-3-carbonyl)glycinate (6)

A solution of 9,9-difluoro-9H-fluorene-3-carboxylic acid (230 mg, 0.93 mmol) in DMF (3 mL) was added methyl glycinate hydrochloride (140 mg, 1.12 mmol), T₃P (1.77 g, 2.79 mmol, 50% wt. in ethyl acetate) and DIPEA (722 mg, 5.58 mmol) under N₂ atmosphere and the reaction mixture was stirred at room temperature for 1 hour. The mixture was diluted with ethyl acetate and washed with saturated aq·NaHCO₃ solution. The aqueous layer was extracted with ethyl acetate twice and the combined organic layers were washed with brine, dried over anhydrous Na₂SO₄, filtered, and concentrated to dryness under reduced pressure. The residue was purified by flash column chromatography on silica gel (petroleum ether:ethyl acetate=5:1) to afford methyl (9,9-difluoro-9H-fluorene-3-carbonyl)glycinate (250 mg, yield 84.7%) as a white solid. LC/MS (ESI) m/z: 318 (M+H)⁺.

### Step 6: (9,9-difluoro-9H-fluorene-3-carbonyl)glycine (7)

A mixture of methyl (9,9-difluoro-9H-fluorene-3-carbonyl)glycinate (250 mg, 0.79 mmol) in MeOH/water (2.5 mL, 4/1) was added LiOH (100 mg, 2.37 mmol) at 0° C., and the reaction mixture was stirred at room temperature for 4 hours. The mixture was acidified with 1 N aq·HCl to pH˜4 and extracted with ethyl acetate twice. The combined organic layers were washed with brine, dried over anhydrous Na₂SO₄, filtered, and concentrated to dryness under reduced pressure to afford (9,9-difluoro-9H-fluorene-3-carbonyl)glycine (230 mg, yield 96.0%) as a white solid. LC/MS (ESI) m/z: 304 (M+H)⁺.

### Step 7: methyl (9,9-difluoro-9H-fluorene-3-carbonyl)glycinate (9)

A solution of (9,9-difluoro-9H-fluorene-3-carbonyl)glycine(90 mg, 0.30 mmol), methyl (S)-1,4-dioxa-7-azaspiro[4.4]nonane-8-carboxylate (169 mg, 0.90 mmol) and DIPEA (233 mg, 1.80 mmol) in DMF (1 mL) was added T₃P (573 mg, 0.90 mmol, 50% wt. in ethyl acetate) under N₂ atmosphere and the reaction mixture was stirred at room temperature for 1 hour. The mixture was diluted with ethyl acetate and washed with saturated aq·NaHCO₃ solution. The aqueous layer was extracted with ethyl acetate twice and the combined organic layers were washed with brine, dried over anhydrous Na₂SO₄, filtered, and concentrated to dryness under reduced pressure. The residue was purified by flash column chromatography on silica gel (petroleum ether:ethyl acetate=3:1) to afford methyl (9,9-difluoro-9H-fluorene-3-carbonyl)glycinate (90 mg, yield 64.1%) as a yellow oil. LC/MS (ESI) m/z: 473 (M+H)⁺.

### Step 8: (S)-7-((9,9-difluoro-9H-fluorene-3-carbonyl)glycyl)-1,4-dioxa-7-azaspiro[4.4]nonane-8-carboxylic acid (10)

A solution of methyl (9,9-difluoro-9H-fluorene-3-carbonyl)glycinate (90 mg, 0.19 mmol) in MeOH/H₂O (2.5 mL, 4/1) was added LiOH (24 mg, 0.57 mmol) and the reaction mixture was stirred at room temperature for 4 hours. The mixture was acidified with 1N aq·HCl to pH˜4 and extracted with ethyl acetate twice. The combined organic layers were washed with brine, dried over anhydrous Na₂SO₄, filtered, and concentrated to dryness under reduced pressure to afford (S)-7-((9,9-difluoro-9H-fluorene-3-carbonyl)glycyl)-1,4-dioxa-7-azaspiro[4.4]nonane-8-carboxylic acid (70 mg, yield 80.4%) as a white solid. LC/MS (ESI) m/z: 459 (M+H)⁺.

### Step 9: (S)—N—((R)-1-(4-carbamimidoylthiophen-2-yl)ethyl)-7-((9,9-difluoro-9H-fluorene-3-carbonyl)glycyl)-1,4-dioxa-7-azaspiro[4.4]nonane-8-carboxamide (Compound 21)

A mixture of (S)-7-((9,9-difluoro-9H-fluorene-3-carbonyl)glycyl)-1,4-dioxa-7-azaspiro [4.4]nonane-8-carboxylic acid (46 mg, 0.10 mmol), (R)-5-(1-aminoethyl)thiophene-3-carboximidamide (26 mg, 0.15 mmol) and DIPEA (78 mg, 0.60 mmol) in DMF (1 mL) was added PyBop (63 mg, 0.12 mmol) at 0° C. under N₂ atmosphere and the reaction mixture was stirred at room temperature for 2 hours. The mixture was diluted with ethyl acetate and washed with saturated aq·NH₄Cl solution. The aqueous layer was extracted with ethyl acetate twice and the combined organic layers were washed with brine, dried over anhydrous Na₂SO₄, filtered, and concentrated to dryness under reduced pressure. The residue was purified by prep-HPLC to afford Compound 21 (4 mg, yield 6.56%) as a white solid. ¹H NMR (400 MHz, CD₃OD) δ 8.24 (dd, J=18.8, 17.4 Hz, 2H), 7.88 (dd, J=7.8, 1.4 Hz, 1H), 7.73 (dd, J=19.4, 7.7 Hz, 2H), 7.65 (d, J=7.4 Hz, 1H), 7.57 (dd, J=18.0, 10.7 Hz, 2H), 7.45 (t, J=7.5 Hz, 1H), 5.35-5.22 (m, 1H), 4.59 (dd, J=8.8, 6.6 Hz, 1H), 4.28-4.13 (m, 2H), 4.06-3.96 (m, 4H), 3.85-3.73 (m, 2H), 2.49-2.37 (m, 1H), 2.26-2.18 (m, 1H), 1.59 (d, J=7.0 Hz, 3H). LC/MS (ESI) m/z: 610 (M+H)⁺.

### Scheme 22: Synthesis of (S)—N—((R)-1-(4-carbamimidoylthiophen-2-yl)ethyl)-7-((4-(p-tolyloxy)benzoyl)glycyl)-1,4-dioxa-7-azaspiro[4.4]nonane-8-carboxamide (Compound 22)

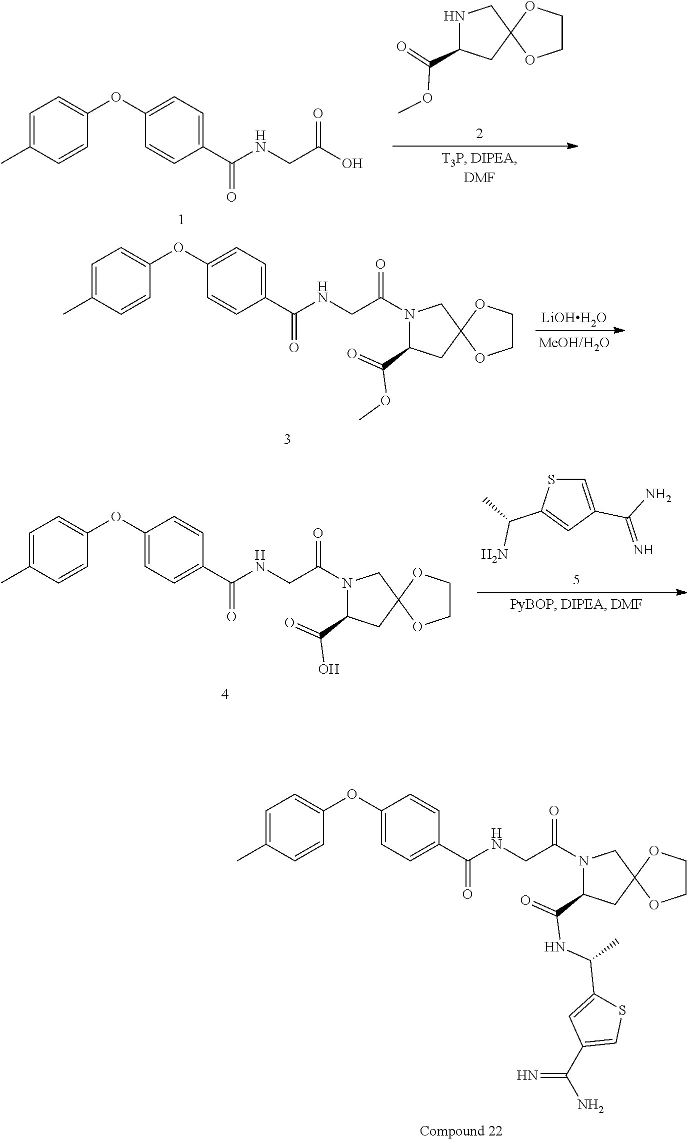

Compound 22 was prepared as a white solid from methyl (S)-1,4-dioxa-7-azaspiro[4.4]nonane-8-carboxylate, (4-(p-tolyloxy)benzoyl)glycine, and (R)-5-(1-aminoethyl)thiophene-3-carboximidamide based on the procedures set forth in Scheme 3. ¹H NMR (400 MHz, CD₃OD) δ 8.53 (s, 1H), 8.24 (dd, J=7.5, 1.6 Hz, 1H), 7.85-7.81 (m, 2H), 7.53 (dd, J=5.3, 4.0 Hz, 1H), 7.24 (d, J=8.1 Hz, 2H), 6.97 (t, J=8.2 Hz, 4H), 5.28 (d, J=6.3 Hz, 1H), 4.57 (dd, J=8.8, 6.6 Hz, 1H), 4.15 (s, 2H), 4.02-3.96 (m, 4H), 3.79 (q, J=10.9 Hz, 2H), 2.43 (dd, J=12.8, 9.0 Hz, 1H), 2.36 (s, 3H), 2.23-2.18 (m, 1H), 1.58 (d, J=7.0 Hz, 3H). LC/MS (ESI) (m/z): 592 (M+H)⁺.

### Scheme 23: Synthesis of (S)—N—((R)-1-(4-carbamimidoylthiophen-2-yl)ethyl)-7-((4-(4-fluorophenoxy)benzoyl)glycyl)-1,4-dioxa-7-azaspiro[4.4]nonane-8-carboxamide (Compoudnd 23)

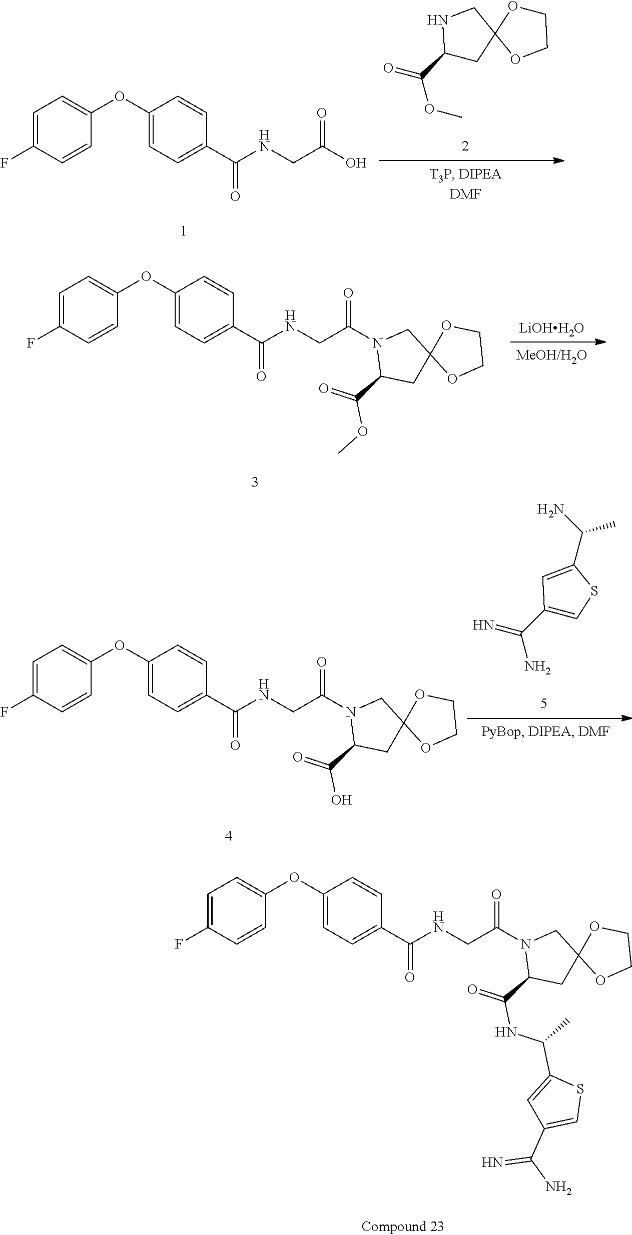

Compound 23 was prepared as a white solid from methyl (S)-1,4-dioxa-7-azaspiro[4.4]nonane-8-carboxylate, (4-(4-fluorophenoxy)benzoyl)glycine, and (R)-5-(1-aminoethyl)thiophene-3-carboximidamide based on the procedures set forth in Scheme 3. ¹H NMR (400 MHz, CD₃OD) δ 8.53 (s, 1H), 8.24 (dd, J=7.5, 1.6 Hz, 1H), 7.85-7.81 (m, 2H), 7.53 (dd, J=5.3, 4.0 Hz, 1H), 7.24 (d, J=8.1 Hz, 2H), 6.97 (t, J=8.2 Hz, 4H), 5.28 (d, J=6.3 Hz, 1H), 4.57 (dd, J=8.8, 6.6 Hz, 1H), 4.15 (s, 2H), 4.02-3.96 (m, 4H), 3.79 (q, J=10.9 Hz, 2H), 2.43 (dd, J=12.8, 9.0 Hz, 1H), 2.36 (s, 3H), 2.23-2.18 (m, 1H), 1.58 (d, J=7.0 Hz, 3H). LC/MS (ESI) (m/z): 592 (M+H)⁺. ¹H NMR (400 MHz, CD₃OD) δ 8.22 (d, J=1.6 Hz, 1H), 7.86-7.83 (m, 2H), 7.52-7.49 (m, 1H), 7.15 (d, J=8.2 Hz, 2H), 7.09 (dd, J=5.8, 3.4 Hz, 2H), 7.00-6.98 (m, 2H), 5.27 (d, J=6.2 Hz, 1H), 4.55 (dd, J=8.9, 6.6 Hz, 1H), 4.14 (s, 2H), 4.01-3.96 (m, 4H), 3.77 (d, J=5.7 Hz, 2H), 2.42 (dd, J=12.8, 9.1 Hz, 1H), 2.20 (dd, J=13.2, 6.4 Hz, 1H), 1.57 (d, J=7.0 Hz, 3H). LC/MS (ESI) (m/z): 596(M+H)⁺.

### Scheme 24: Synthesis of (2S,4R)—N—((R)-1-(4-carbamimidoylthiophen-2-yl)ethyl)-4-(difluoromethoxy)-1-((3-methyl-4-phenoxybenzoyl)glycyl)pyrrolidine-2-carboxamide (Compound 24)

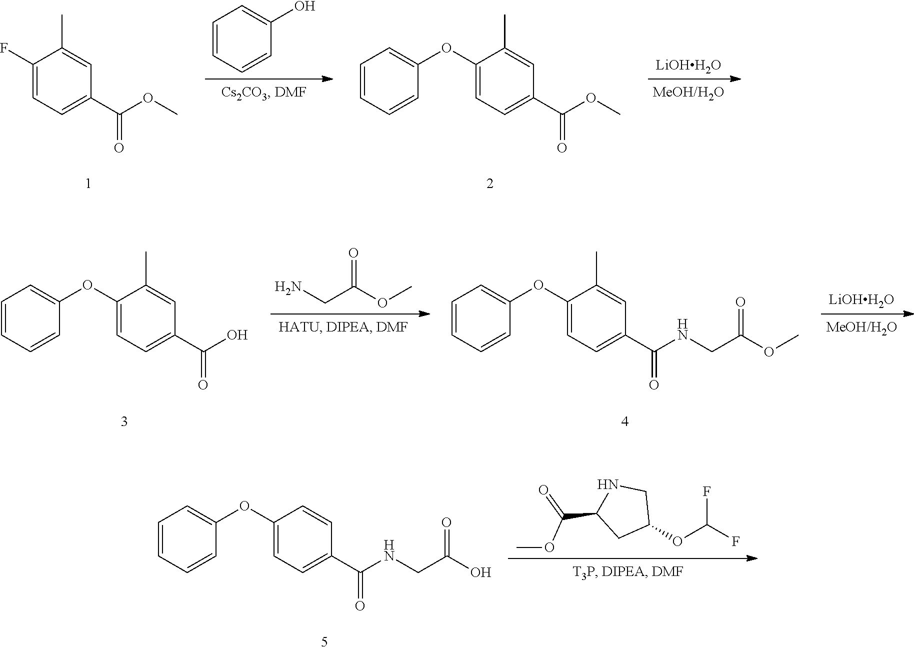

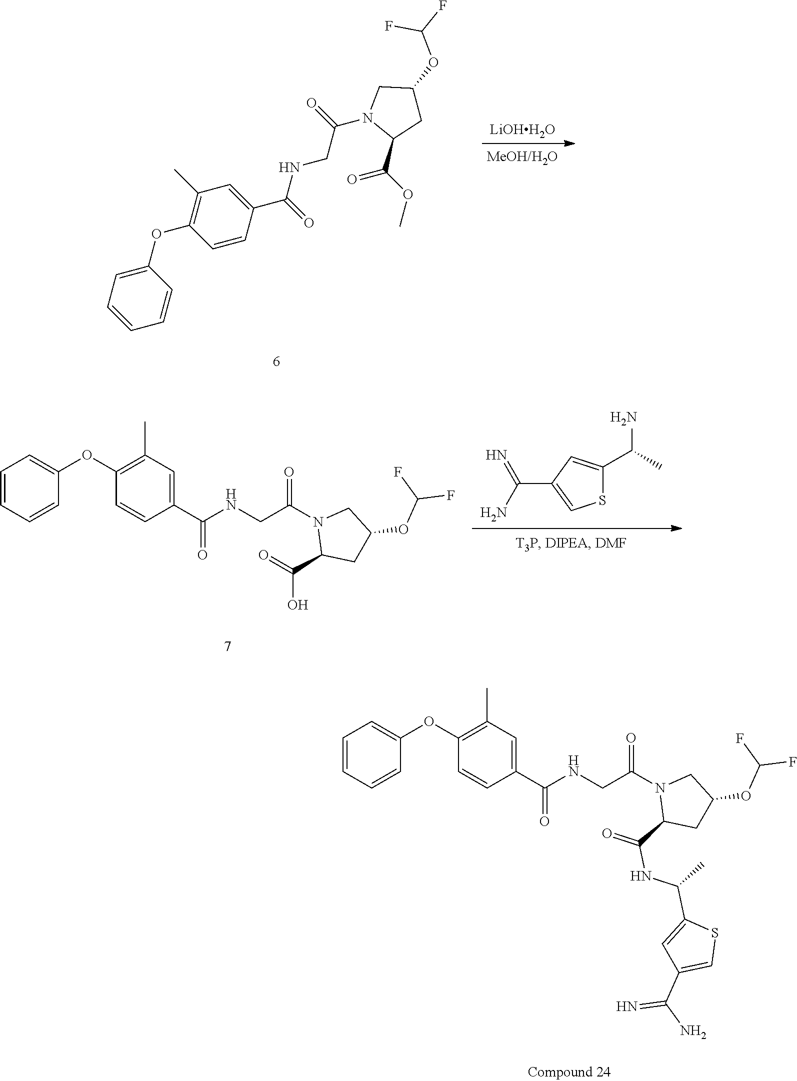 [SMILES: COC(=O)[C@@H]1C[C@@H](OC(F)F)CN1C(=O)CNC(=O)c1ccc(Oc2ccccc2)c(C)c1 ; "LiOH·H2O" ; Cc1cc(C(=O)NCC(=O)N2C[C@H](OC(F)F)C[C@H]2C(=O)O)ccc1Oc1ccccc1 ; C[C@@H](N)c1cc(C(=N)N)cs1 ; Cc1cc(C(=O)NCC(=O)N2C[C@H](OC(F)F)C[C@H]2C(=O)N[C@H](C)c2cc(C(=N)N)cs2)ccc1Oc1ccccc1]

### Step 1: methyl (2S,4R)-4-phenoxy-1-((4-phenoxybenzoyl)glycyl)pyrrolidine-2-carboxylate (2)

To a mixture of methyl 4-fluoro-3-methylbenzoate (3.0 g, 17.84 mmol) and phenol (1.68 g, 17.84 mmol) in DMF (30 mL) was added Cs₂CO₃ (17.45 g, 53.52 mmol) and the mixture was stirred at 120° C. for 16 hours. The mixture was diluted with ethyl acetate and washed with water twice. The combined organic layers were dried over anhydrous Na₂SO₄, filtered, and concentrated to dryness under reduced pressure. The residue was purified by flash column chromatography on silica gel (petroleum ether:ethyl acetate=98:2) to give methyl 3-methyl-4-phenoxybenzoate (1.37 g, yield 32.0%) as a colorless oil. LC/MS (ESI) m/z: 243 (M+H)⁺.

### Step 2: 3-methyl-4-phenoxybenzoic acid (3)

To a solution of methyl 3-methyl-4-phenoxybenzoate (1.37 g, 5.66 mmol) in MeOH (5 mL) and water (1 mL) was added lithium hydroxide (713 mg, 16.98 mmol) at 0° C. and the mixture was stirred at room temperature for 2 hours. The mixture was acidified with 1 N aq·HCl solution to pH-3, extracted with ethyl acetate twice. The combined organic layers were washed with brine, dried over anhydrous Na₂SO₄, filtered, and concentrated to dryness under reduced pressure to give 3-methyl-4-phenoxybenzoic acid (1.2 g, yield 93.0%) as a white solid, which was used directly in the next step. LC/MS (ESI) (m/z): 229 (M+H)⁺.

### Step 3: methyl (3-methyl-4-phenoxybenzoyl)glycinate (4)

To a mixture of 3-methyl-4-phenoxybenzoic acid (1.2 g, 5.26 mmol) and methyl glycinate (990 mg, 7.89 mmol) in DMF (15.0 mL) was added DIPEA (4.34 mL, 0.26.3 mmol) and HATU (3.0 g, 7.89 mmol) at 0° C. under N₂ atmosphere and the mixture was stirred at 25° C. for 1 hour. The mixture was diluted with ethyl acetate and washed with water twice. The combined organic layers were dried over anhydrous Na₂SO₄, filtered, and concentrated to dryness under reduced pressure. The residue was purified by flash column chromatography on silica gel (petroleum ether:ethyl acetate=3:2) to give methyl (3-methyl-4-phenoxybenzoyl) glycinate (1.5 g, yield 95.5%) as a colorless oil. LC/MS (ESI) m/z: 300 (M+H)⁺.

### Step 4: (3-methyl-4-phenoxybenzoyl)glycine (5)

To a solution of methyl (3-methyl-4-phenoxybenzoyl)glycinate (1.62 g, 5.42 mmol) in MeOH (10 mL) and water (2 mL) was added lithium hydroxide (618 mg, 16.25 mmol) at 0° C. and the mixture was stirred at room temperature for 2 hours. The mixture was acidified with 1 N aq·HCl solution to pH-3, extracted with ethyl acetate twice. The combined organic layers were washed with brine, dried over anhydrous Na₂SO₄, filtered, and concentrated to dryness under reduced pressure to give (3-methyl-4-phenoxybenzoyl)glycine (1.24 g, yield 80.5%) as a white solid, which was used directly in the next step. LC/MS (ESI) (m/z): 286 (M+H)⁺.

### Step 5: methyl (2S,4R)-4-(difluoromethoxy)-1-((3-methyl-4-phenoxybenzoyl)glycy) pyrrolidine-2-carboxylate (6)

To a mixture of (3-methyl-4-phenoxybenzoyl)glycine (100 mg, 0.35 mmol) and methyl (2S,4R)-4-(difluoromethoxy)pyrrolidine-2-carboxylate hydrochloride (100 mg, 0.53 mmol) in DMF (3.0 mL) was added DIPEA (0.35 mL, 2.10 mmol) and T₃P (335 mg, 0.53 mmol, 50% wt. in ethyl acetate) at 0° C. under N₂ atmosphere and the mixture was stirred at 25° C. for 16 hours. The mixture was diluted with water and extracted with ethyl acetate twice. The combined organic layers were dried over anhydrous Na₂SO₄, filtered, and concentrated to dryness under reduced pressure. The residue was purified by flash column chromatography on silica gel (dichloromethane:methanol=97:3) to give methyl (2S,4R)-4-(difluoromethoxy)-1-((3-methyl-4-phenoxybenzoyl)glycyl)pyrrolidine-2-carboxylate (160 mg, yield 98.8%) as a yellow oil. LC/MS (ESI) m/z: 463 (M+H)⁺.

### Step 6: (2S,4R)-4-(difluoromethoxy)-1-((3-methyl-4-phenoxybenzoyl)glycyl)pyrrolidine-2-carboxylic acid (7)

To a solution of methyl (2S,4R)-4-(difluoromethoxy)-1-((3-methyl-4-phenoxybenzoyl) glycyl)pyrrolidine-2-carboxylate (160 mg, 0.35 mmol) in methanol (3 mL) and water (0.6 mL) was added lithium hydroxide (22 mg, 0.53 mmol) at 0° C. and the mixture was stirred at room temperature for 2 hours. The mixture was acidified with 1 N aq·HCl solution to pH ˜3, extracted with ethyl acetate twice. The combined organic layers were washed with brine, dried over anhydrous Na₂SO₄, filtered, and concentrated to dryness under reduced pressure to give (2S,4R)-4-(difluoromethoxy)-1-((3-methyl-4-phenoxybenzoyl)glycyl)pyrrolidine-2-carboxylic acid (113 mg, yield 72.9%) as a colorless oil, which was used directly in the next step. LC/MS (ESI) (m/z): 449 (M+H)⁺.

### Step 7: (2S,4R)—N—((R)-1-(4-carbamimidoylthiophen-2-yl)ethyl)-4-(difluoromethoxy)-1-((3-methyl-4-phenoxybenzoyl)glycyl)pyrrolidine-2-carboxamide (Compound 24)

To a mixture of (2S,4R)-4-(difluoromethoxy)-1-((3-methyl-4-phenoxybenzoyl)glycyl) pyrrolidine-2-carboxylic acid (50 mg, 0.11 mmol) and (R)-5-(1-aminoethyl)thiophene-3-carboximidamide (38 mg, 0.22 mmol) in DMF (3.0 mL) was added DIPEA (0.1 mL, 0.55 mmol) and T₃P (87 mg, 0.13 mmol, 50% wt. in ethyl acetate) at 0° C. under N₂ atmosphere and the mixture was stirred at 25° C. for 16 hours. The mixture was concentrated to dryness under reduced pressure. The residue was purified by prep-TLC (dichloromethane:methanol=10:1) and further purified by prep-HPLC to give Compound 24 (5 mg, yield 7.6%) as a white solid. ¹H NMR (400 MHz, CD₃OD) δ 8.55 (s, 1H), 8.23 (dd, J=10.3, 1.6 Hz, 1H), 7.80 (t, J=4.2 Hz, 1H), 7.65 (dt, J=8.0, 4.0 Hz, 1H), 7.53 (dd, J=7.1, 5.9 Hz, 1H), 7.41-7.35 (m, 2H), 7.15 (t, J=7.4 Hz, 1H), 6.97 (dd, J=8.6, 1.0 Hz, 2H), 6.83 (d, J=8.5 Hz, 1H), 6.51 (dd, J=82.4, 66.5 Hz, 1H), 5.38-5.23 (m, 1H), 5.02 (s, 1H), 4.66-4.54 (m, 1H), 4.18 (dt, J=33.9, 16.9 Hz, 2H), 3.98-3.85 (m, 2H), 2.64-2.44 (m, 1H), 2.32 (d, J=2.8 Hz, 3H), 2.29-2.12 (m, 1H), 1.62 (dd, J=29.7, 7.0 Hz, 3H). LC/MS (ESI) m/z: 600 (M+H)⁺.

### Scheme 25: Synthesis of (1S,3S,5S)—N—((R)-1-(4-carbamimidoylthiophen-2-yl) ethyl)-5-methyl-2-((3-methyl-4-(p-tolyloxy)benzoyl)glycyl)-2-azabicyclo[3.1.]hexane-3-carboxamide(Compound 25)

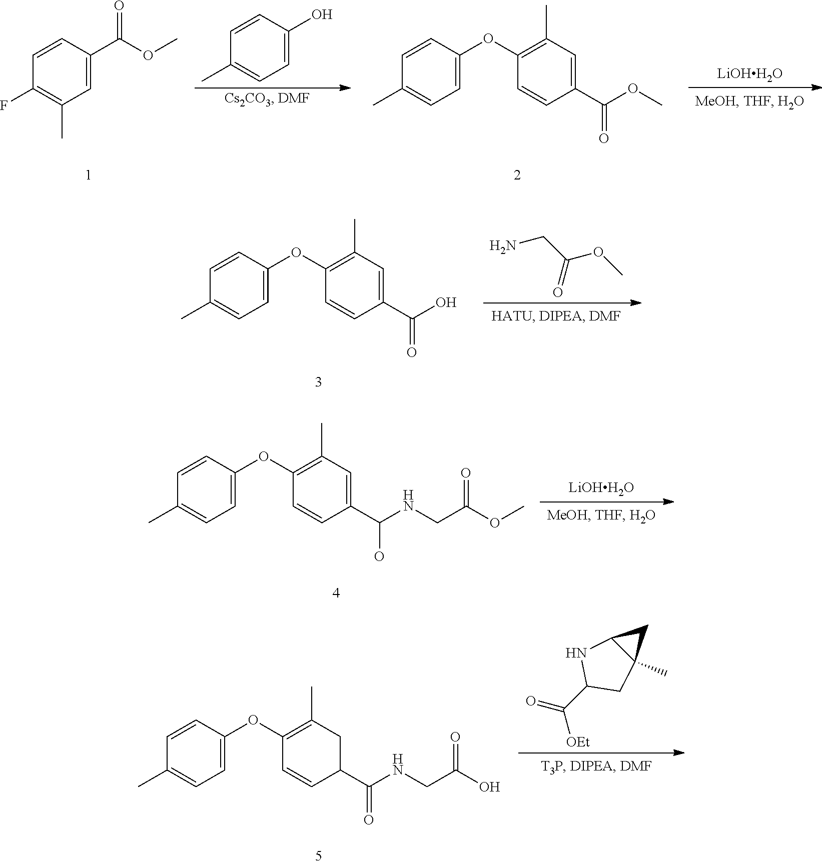

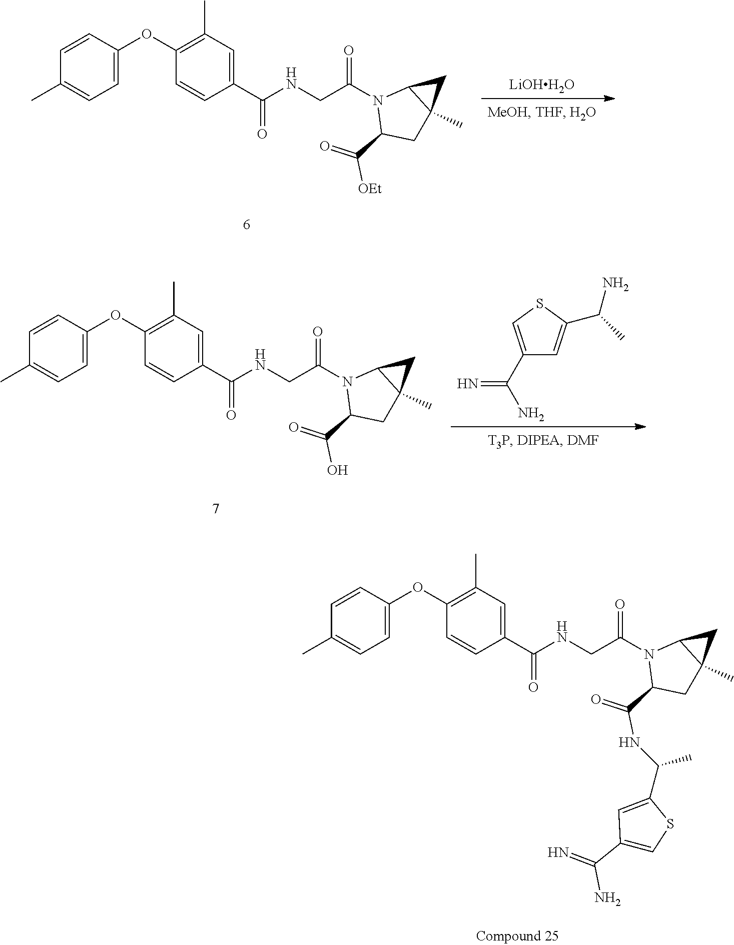

Compound 25 was prepared as a white solid from 4-fluoro-3-methylbenzoate, p-cresol, methyl glycinate hydrochloride, ethyl (1 S,3S,5S)-5-methyl-2-azabicyclo[3.1.0]hexane-3-carboxylate, and (R)-5-(1-aminoethyl)thiophene-3-carboximidamide based on the procedures set forth in Scheme 24. ¹H NMR (400 MHz, CD₃OD) δ 8.46 (s, 1H), 8.14 (dd, J=9.2, 9.2 Hz, 1H), 7.69 (d, J=1.6 Hz, 1H), 7.53 (dd, J=1.6, 9.2 Hz, 1H), 7.44-7.38 (m, 1H), 7.11 (d, J=8.0 Hz, 2H), 6.78 (d, J=8.4 Hz, 2H), 6.68 (d, J=8.4 Hz, 1H), 5.17-5.10 (m, 1H), 4.74 (d, J=4.4 Hz, 1H), 4.31 (d, J=16.4 Hz, 1H), 4.17 (d, J=16.4 Hz, 1H), 3.31 (dd, J=2.8, 5.6 Hz, 1H), 2.35 (t, J=12.4 Hz, 1H), 2.24 (d, J=8.0 Hz, 6H), 2.07 (dd, J=13.6, 13.2 Hz, 1H), 1.49 (d, J=6.8 Hz, 3H), 1.21 (s, 3H), 1.11 (dd, J=2.4, 5.6 Hz, 1H), 0.69 (t, J=5.4 Hz, 1H). LC/MS (ESI) (m/z):574(M+H)⁺.

### Scheme 26: Synthesis of (1S,3S,5S)—N—((R)-1-(5-carbamimidoylthiophen-2-yl) ethyl)-5-methyl-2-((4-(p-tolyloxy)benzoyl)glycyl)-2-azabicyclo[3.1.0]hexane-3-carboxamide (Compound 26)

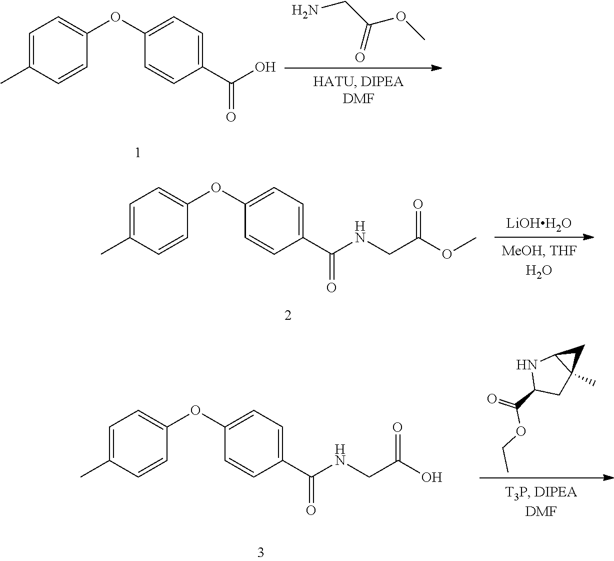

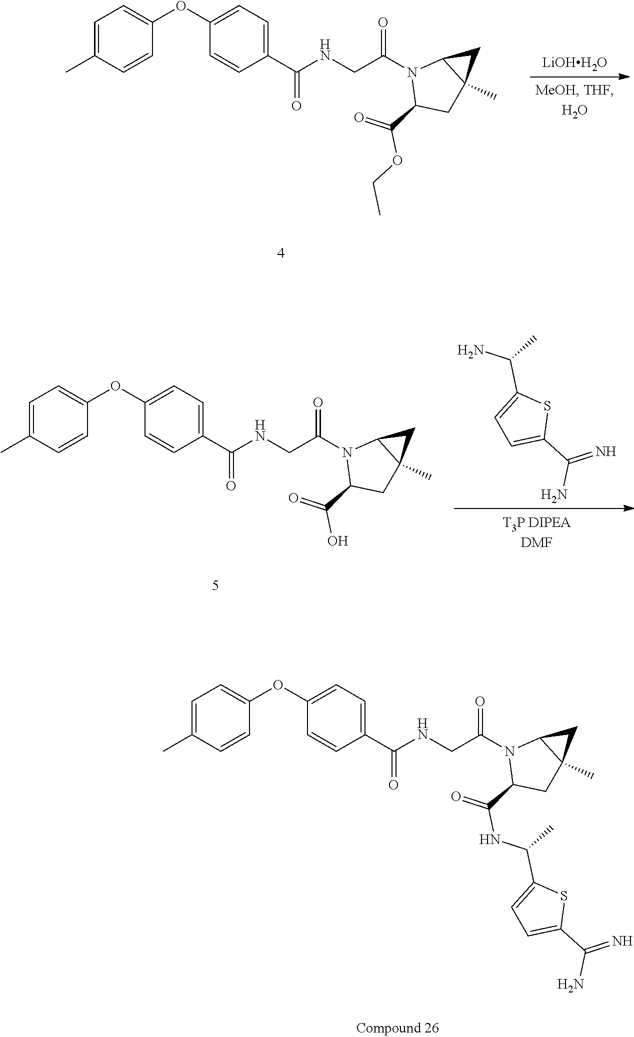

Compound 26 was prepared as a white solid from 4-(p-tolyloxy)benzoic acid, methyl glycinate, ethyl (1S,3S,5S)-5-methyl-2-azabicyclo[3.1.0]hexane-3-carboxylate, and (R)-5-(1-aminoethyl)thiophene-2-carboximidamide based on the procedures set forth in Scheme 24. ¹H NMR (400 MHz, CD₃OD) δ 8.50 (s, 1H), 7.85-7.77 (m, 3H), 7.24-7.18 (m, 3H), 6.98-6.93 (m, 4H), 5.25 (q, J=14 Hz, 1H), 4.83 (d, J=3.6 Hz, 1H), 4.44 (d, J=16.4 Hz, 1H), 4.23 (d, J=16.8 Hz, 1H), 3.39 (dd, J=6.0, 6.0 Hz, 1H), 2.43 (t, J=13.0 Hz, 1H), 2.35 (s, 3H), 2.14 (dd, J=13.6, 3.6 Hz, 1H), 1.58 (d, J=6.8 Hz, 3H), 1.30 (s, 3H), 1.23 (dd, J=5.6, 2.4 Hz, 1H), 0.78 (t, J=5.2 Hz, 1H). LC/MS (ESI) m/z: 560 (M+H)₊.

### Scheme 27: Synthesis of (1S,3S,5S)—N—((R)-1-(4-carbamimidoylthiophen-2-yl) ethyl)-2-((4-(4-fluorophenoxy)-3-methylbenzoyl)glycyl)-5-methyl-2-azabicyclo[3.1.0]hexane-3-carboxamide (Compound 27)

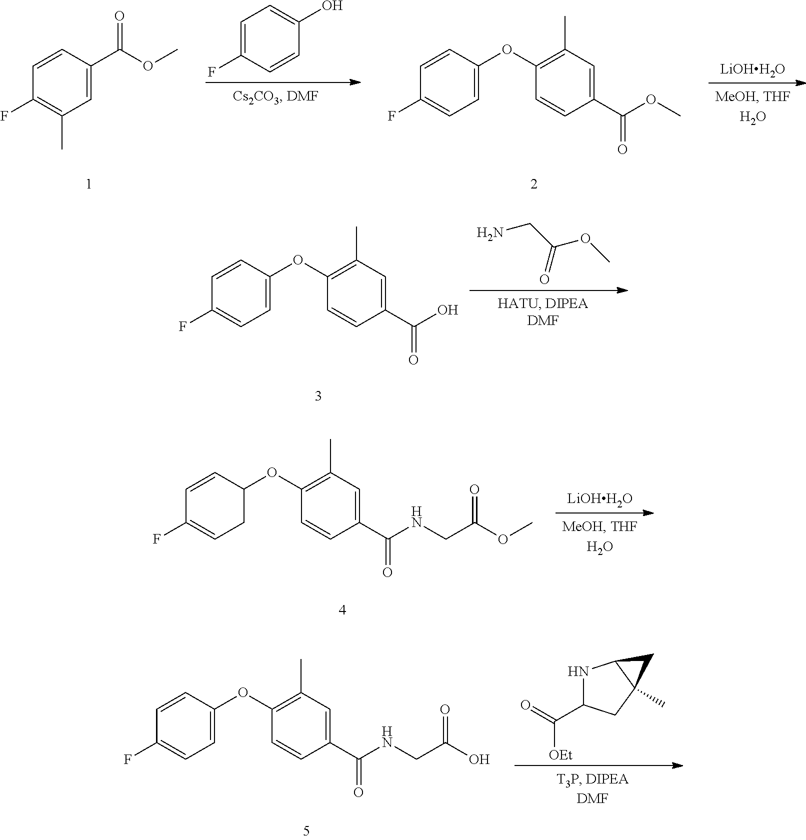

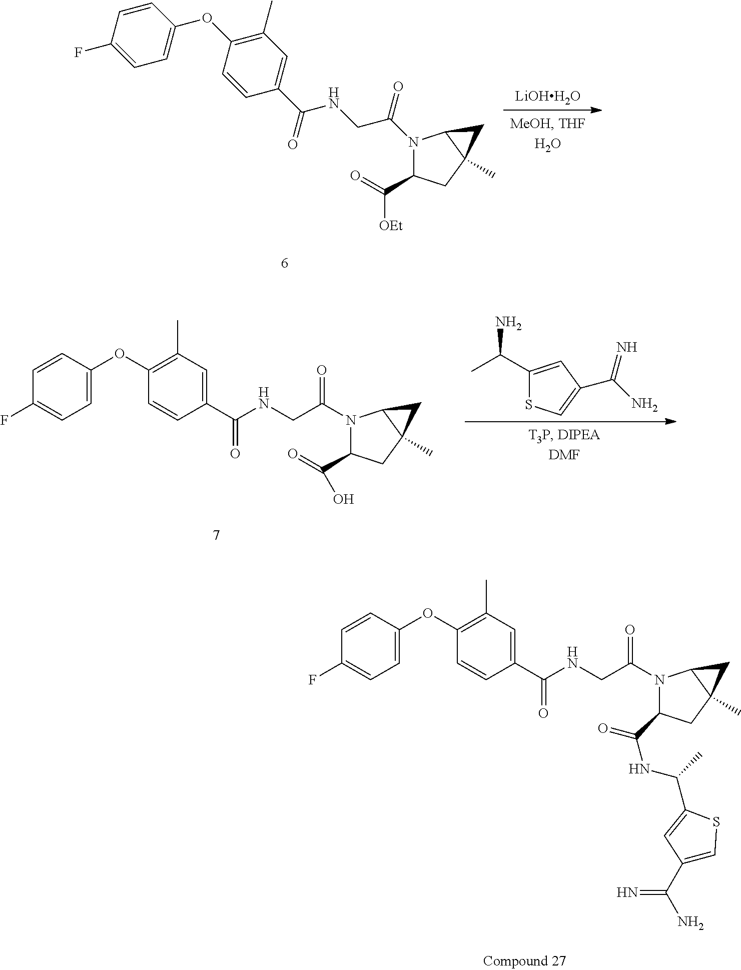

Compound 27 was prepared as a white solid from methyl 4-fluoro-3-methylbenzoate, 4-fluorophenol, methyl glycinate, ethyl (1 S,3S,5S)-5-methyl-2-azabicyclo[3.1.0]hexane-3-carboxylate, and (R)-5-(1-aminoethyl) thiophene-3-carboximidamide based on the procedures set forth in Scheme 24. ¹H NMR (400 MHz, CD₃OD) δ 8.50 (s, 1H), 8.16 (d, J=1.6 Hz, 1H), 7.73 (d, J=2.0 Hz, 1H), 7.58 (dd, J=8.8, 3.0 Hz, 1H), 7.44 (s, 1H), 7.11-7.04 (m, 2H), 6.97-6.91 (m, 2H), 6.73 (d, J=2.4 Hz, 1H), 5.16 (q, J=6.8 Hz, 1H), 4.76 (d, J=3.6 Hz, 1H), 4.34 (d, J=16.4 Hz, 1H), 4.20 (d, J=16.4 Hz, 1H), 3.33 (dd, J=6.0, 2.0 Hz, 1H), 2.38 (t, J=12.8 Hz, 1H), 2.26 (s, 3H), 2.10 (dd, J=10.4, 2.4 Hz, 1H), 1.52 (d, J=7.2 Hz, 3H), 1.24 (s, 3H), 1.13 (dd, J=5.6, 2.4 Hz, 1H), 0.71 (t, J=5.6 Hz, 1H). LC/MS (ESI) m/z: 578 (M+H)⁺.

### Scheme 28: Synthesis of (1S,3S,5S)—N—((R)-1-(4-carbamimidoylthiophen-2-yl)ethyl)-5-methyl-2-((3-methyl-4-phenoxybenzoyl)glycyl)-2-azabicyclo[3.1.0]hexane-3-carboxamide (Compound 28)

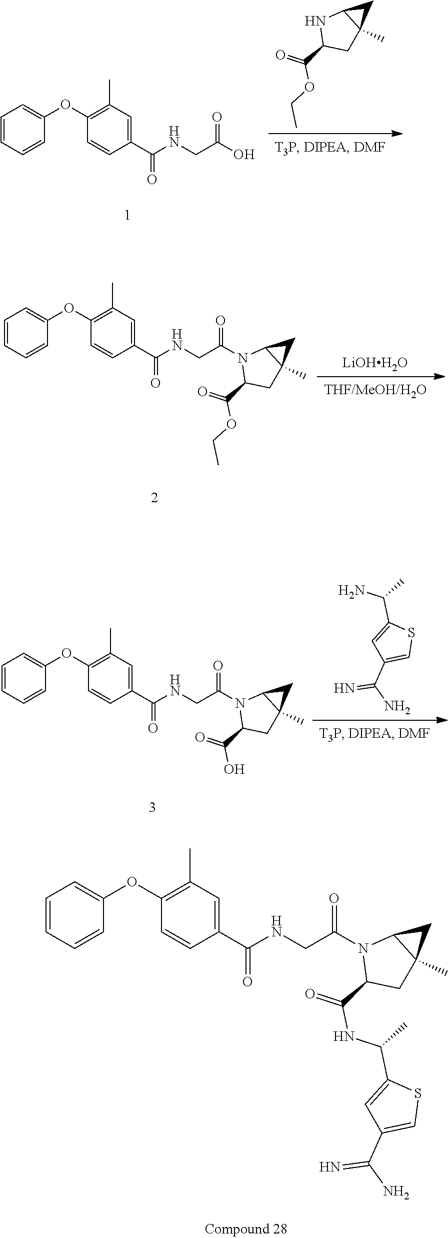

Compound 28 was prepared as a white solid from (3-methyl-4-phenoxybenzoyl)glycine, ethyl (1S,3S,5S)-5-methyl-2-azabicyclo[3.1.0]hexane-3-carboxylate, and (R)-5-(1-aminoethyl) thiophene-3-carboximidamide based on the procedures set forth in Scheme 4. ¹H NMR (400 MHz, CD₃OD) δ 8.55 (s, 1H), 8.23 (dd, J=9.3, 1.6 Hz, 1H), 7.80 (d, J=1.7 Hz, 1H), 7.65 (dd, J=8.7, 2.2 Hz, 1H), 7.50 (s, 1H), 7.40-7.35 (m, 2H), 7.15 (t, J=7.4 Hz, 1H), 6.97 (d, J=7.8 Hz, 2H), 6.82 (d, J=8.5 Hz, 1H), 5.23 (q, J=6.9 Hz, 1H), 4.82 (d, J=3.6 Hz, 1H), 4.33 (dd, J=56.5, 16.4 Hz, 2H), 3.42 (dd, J=19.4, 17.0 Hz, 1H), 2.44 (t, J=12.7 Hz, 1H), 2.31 (s, 3H), 2.16 (dd, J=13.3, 3.5 Hz, 1H), 1.60 (t, J=11.3 Hz, 3H), 1.29 (d, J=12.0 Hz, 3H), 1.22-1.17 (m, 1H), 0.78 (t, J=5.7 Hz, 1H). LC/MS (ESI) m/z: 560 (M+H)⁺.

### Scheme 29: Synthesis of (1S,3S,5S)—N—((R)-1-(5-carbamimidoylthiophen-2-yl)ethyl)-2-((9,9-difluoro-9H-fluorene-3-carbonyl)glycyl)-5-methyl-2-azabicyclo[3.1.0]hexane-3-carboxamide (Compound 29)

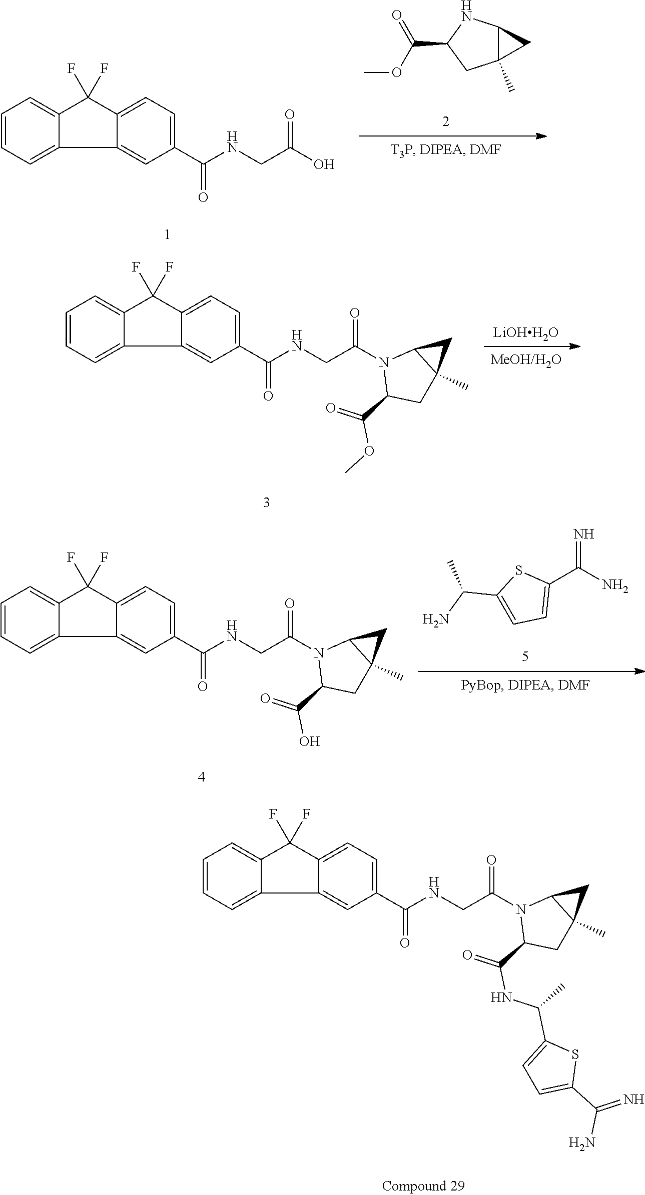

Compound 29 was prepared as a white solid from (9,9-difluoro-9H-fluorene-3-carbonyl)glycine, methyl (1S,3S,5S)-5-methyl-2-azabicyclo[3.1.0]hexane-3-carboxylate, and (R)-5-(1-aminoethyl)thiophene-2-carboximidamide based on the procedures set forth in Scheme 3. ¹H NMR (400 MHz, CD₃OD) δ 8.51 (s, 1H), 8.20 (s, 1H), 7.89 (dd, J=7.9, 1.4 Hz, 1H), 7.77-7.70 (m, 3H), 7.65 (d, J=7.2 Hz, 1H), 7.58 (t, J=7.5 Hz, 1H), 7.45 (t, J=7.5 Hz, 1H), 7.20 (d, J=3.3 Hz, 1H), 5.26 (d, J=7.0 Hz, 1H), 4.82 (d, J=3.5 Hz, 1H), 4.53 (d, J=16.7 Hz, 1H), 4.28 (d, J=16.6 Hz, 1H), 3.41 (dd, J=6.0, 2.3 Hz, 1H), 2.44 (d, J=12.6 Hz, 1H), 2.15 (dd, J=13.3, 3.6 Hz, 1H), 1.59 (d, J=7.0 Hz, 3H), 1.32 (s, 3H), 1.10 (d, J=6.6 Hz, 1H), 0.82 (t, J=5.2 Hz, 1H). LC/MS (ESI) m/z: 578 (M+H)⁺.

### Scheme 30: Synthesis of (2S,4R)—N—((R)-1-(5-carbamimidoylthiophen-2-yl)ethyl)-1-((9,9-difluoro-9H-fluorene-3-carbonyl)glycyl)-4-(difluoromethoxy)pyrrolidine-2-carboxamide (Compound 30)

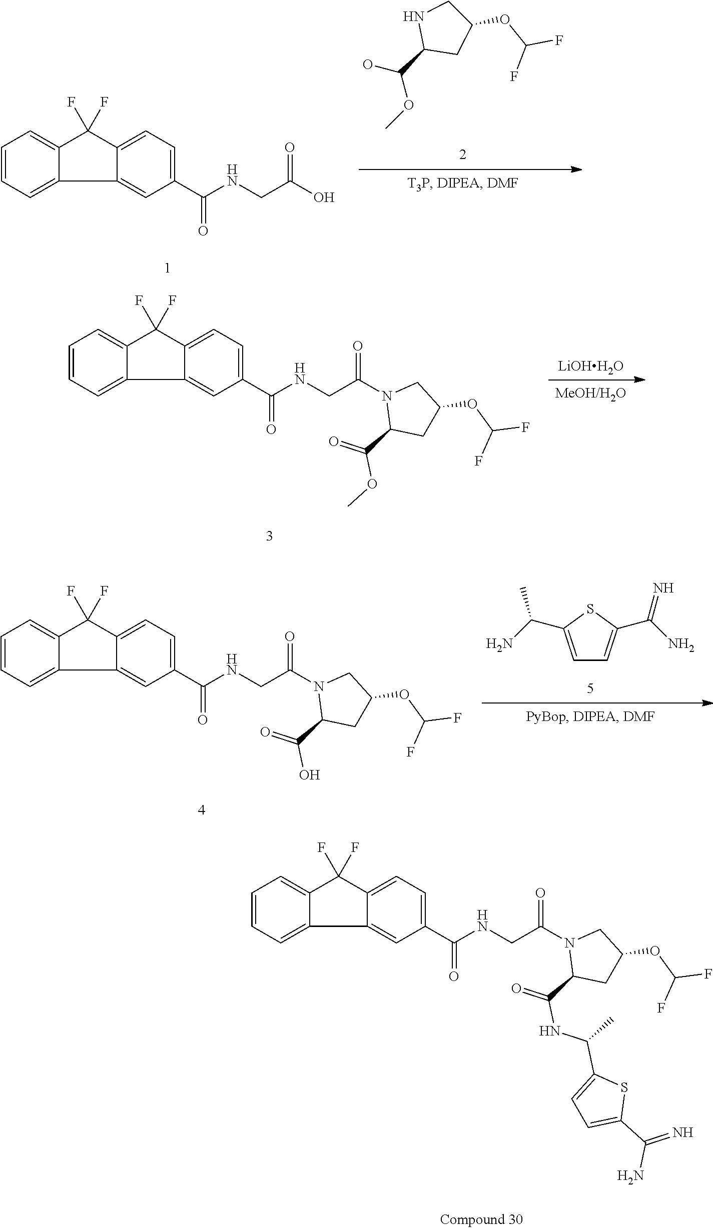

Compound 30 was prepared as a white solid from (9,9-difluoro-9H-fluorene-3-carbonyl)glycine, methyl (2S,4R)-4-(difluoromethoxy)pyrrolidine-2-carboxylate, and (R)-5-(1-aminoethyl)thiophene-2-carboximidamide based on the procedures set forth in Scheme 3. ¹H NMR (400 MHz, CD₃OD) δ 8.53 (s, 2H), 8.20 (s, 1H), 7.90 (dd, J=7.8, 1.4 Hz, 1H), 7.78-7.70 (m, 3H), 7.65 (d, J=7.3 Hz, 1H), 7.58 (t, J=7.5 Hz, 1H), 7.45 (t, J=7.5 Hz, 1H), 7.25 (dd, J=11.5, 4.1 Hz, 1H), 6.53 (dd, J=76.5, 72.5 Hz, 1H), 5.31 (dd, J=13.9, 7.2 Hz, 1H), 5.05 (s, 1H), 4.59 (t, J=7.8 Hz, 1H), 4.36 (d, J=16.8 Hz, 1H), 4.18 (d, J=16.8 Hz, 1H), 3.93 (dd, J=22.8, 7.1 Hz, 2H), 2.53-2.44 (m, 1H), 2.28-2.20 (m, 1H), 1.60 (d, J=7.0 Hz, 3H). LC/MS (ESI) m/z: 618 (M+H)⁺.

### Scheme 31: Synthesis of (1S,3S,5S)—N—((R)-1-(5-carbamimidoylthiophen-2-yl)ethyl)-5-methyl-2-((3-methyl-4-phenoxybenzoyl)glycyl)-2-azabicyclo[3.1.0]hexane-3-carboxamide (Compound 31)

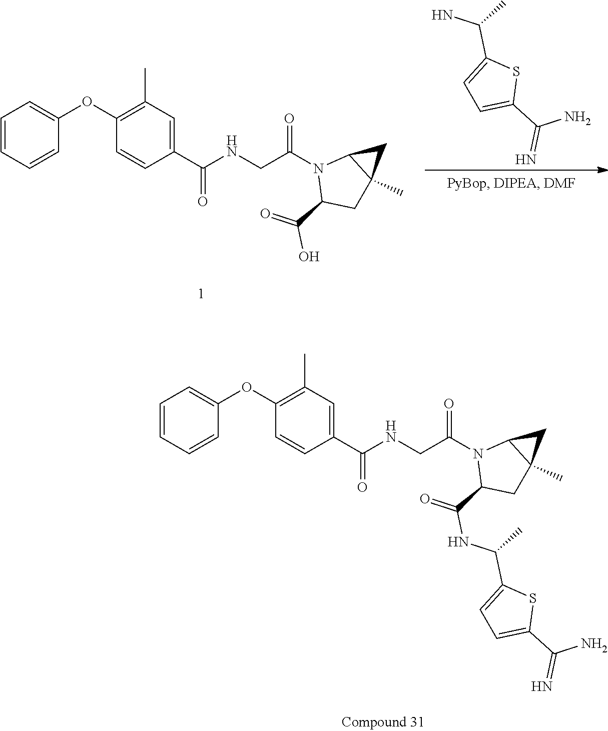 [SMILES: Cc1cc(C(=O)NCC(=O)N2[C@H]3C[C@@]3(C)C[C@H]2C(=O)O)ccc1Oc1ccccc1 ; C[C@@H](N)c1ccc(C(=N)N)s1 ; Cc1cc(C(=O)NCC(=O)N2[C@H]3C[C@@]3(C)C[C@H]2C(=O)N[C@H](C)c2ccc(C(=N)N)s2)ccc1Oc1ccccc1]

Compound 31 was prepared as a white solid from (1S,3S,5S)-5-methyl-2-((3-methyl-4-phenoxybenzoyl)glycyl)-2-azabicyclo[3.1.0]hexane-3-carboxylic acid and (R)-5-(1-aminoethyl)thiophene-2-carboximidamide based on the procedures set forth in Scheme 3. ¹H-NMR (400 MHz, CD₃OD) δ 8.61 (s, 1H), 7.85-7.58 (m, 3H), 7.42-7.32 (m, 2H), 7.25-7.07 (m, 2H), 6.96 (d, J=7.8 Hz, 2H), 6.85-6.77 (m, 1H), 5.29 (dq, J=30.1, 7.0 Hz, 1H), 4.84-4.80 (m, 1H), 4.53-4.17 (m, 2H), 3.46 (ddd, J=45.0, 8.3, 3.3 Hz, 1H), 2.65-2.40 (m, 1H), 2.19-2.03 (m, 1H), 1.74-1.55 (m, 3H), 1.37-1.19 (m, 4H), 0.89-0.69 (m, 1H). LC/MS (ESI) m/z:560 (M+H)⁺.

### Scheme 32: (2S,4R)—N—((R)-1-(4-carbamimidoylthiophen-2-yl)ethyl)-4-fluoro-4-(fluoromethyl)-1-((3-methyl-4-phenoxybenzoyl)glycyl)pyrrolidine-2-carboxamide (Compound 32)

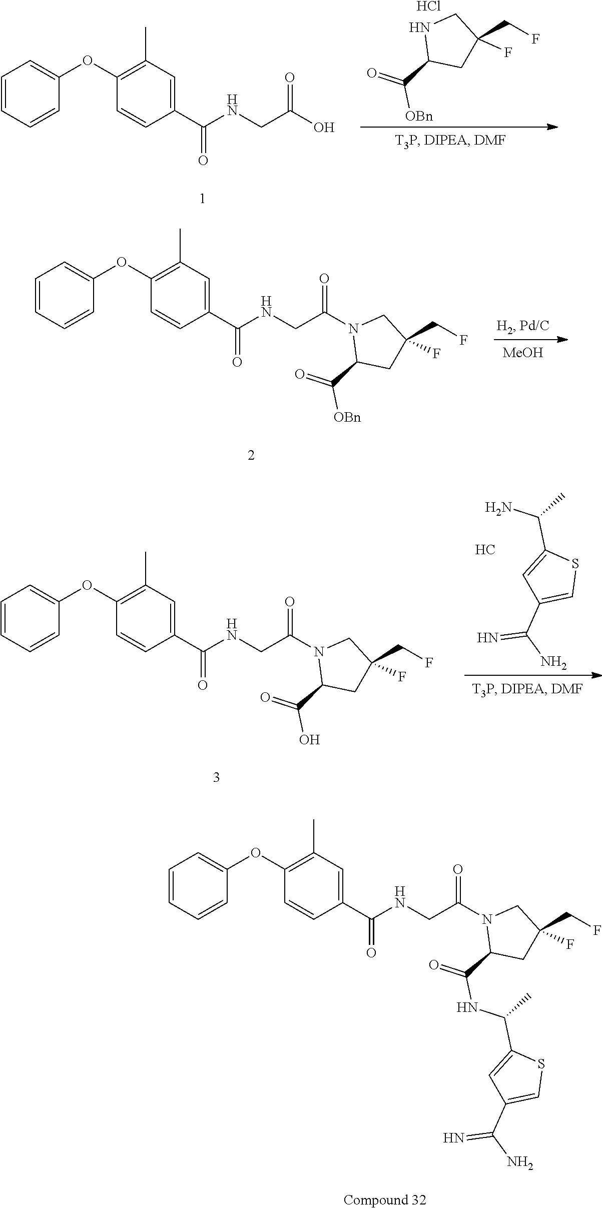

### Step 1: benzyl (2S,4R)-4-fluoro-4-(fluoromethyl)-1-((3-methyl-4-phenoxybenzoyl)glycyl) pyrrolidine-2-carboxylate

At 0° C., to a mixture of (3-methyl-4-phenoxybenzoyl)glycine (38 mg, 0.132 mmol) and benzyl (2S,4R)-4-fluoro-4-(fluoromethyl)pyrrolidine-2-carboxylate (30 mg, 0.12 mmol) in N,N-dimethylformamide (2 mL) was added propylphosphonic anhydride (229 mg, 0.36 mmol, 50% wt. in ethyl acetate) and N,N-diisopropylethylamine (78 mg, 0.6 mmol) under nitrogen atmosphere and the mixture was stirred at room temperature for 2 hours. The mixture was diluted with ethyl acetate and washed with saturated sodium bicarbonate solution. The aqueous layer was extracted with ethyl acetate twice and the combined organic layers were washed with brine, dried over anhydrous sodium sulfate, filtered, and concentrated to dryness under reduced pressure. The residue was purified by flash chromatography on silica gel (petroleum ether:ethyl acetate=1:1) to afford benzyl (2S,4R)-4-fluoro-4-(fluoromethyl)-1-((3-methyl-4-phenoxybenzoyl)glycyl)pyrrolidine-2-carboxylate (60 mg, yield 98.3%) as a colorless oil. LC/MS (ESI) m/z: 523 (M+H)⁺.

### Step 2: (2S,4R)-4-fluoro-4-(fluoromethyl)-1-((3-methyl-4-phenoxybenzoyl)glycyl) pyrrolidine-2-carboxylic acid

To a solution of benzyl (2S,4R)-4-fluoro-4-(fluoromethyl)-1-((3-methyl-4-phenoxybenzoyl) glycyl)pyrrolidine-2-carboxylate (60 mg, 0.11 mmol) in methanol (2 mL) was added Pd/C (10 mg, 10% wt.) at 0° C., and the reaction mixture was stirred under hydrogen atmosphere at 25° C. for 2 hours. The mixture was filtered, and the filtrate was concentrated to dryness under reduced pressure give (2S,4R)-4-fluoro-4-(fluoromethyl)-1-((3-methyl-4-phenoxybenzoyl) glycyl)pyrrolidine-2-carboxylic acid (43 mg, yield 86.5%) as a white solid, which was used directly in the next step. LC/MS (ESI) m/z: 433 (M+H)⁺.

### Step 3: (2S,4R)—N—((R)-1-(4-carbamimidoylthiophen-2-yl)ethyl)-4-fluoro-4-(fluoromethyl)-1-((3-methyl-4-phenoxybenzoyl)glycyl)pyrrolidine-2-carboxamide (Compound 32)

To a mixture of (2S,4R)-4-fluoro-4-(fluoromethyl)-1-((3-methyl-4-phenoxybenzoyl)glycyl) pyrrolidine-2-carboxylic acid (40 mg, 0.093 mmol) and (R)-5-(1-aminoethyl)thiophene-3-carboximidamide (18 mg, 0.102 mmol) in N,N-dimethylformamide (2.0 mL) was added N,N-diisopropylethylamine (60 mg, 0.465 mmol) and T3P (71 mg, 0.11 mmol, 50% wt. in ethyl acetate) at 0° C. under nitrogen atmosphere and the mixture was stirred at 25° C. for 2 hours. The mixture was concentrated to dryness under reduced pressure. The residue was purified by prep-TLC (dichloromethane:methanol=10:1) to give Compound 32 (1.0 mg, yield 1.85%) as a white solid. ¹HNMR (400 MHz, CD₃OD) δ 8.47 (s, 2H), 8.18 (d, J=8 Hz, 1H), 7.74 (d, J=8 Hz, 1H), 7.60 (d, J=8 Hz, 1H), 7.50 (d, J=8 Hz, 1H), 7.32 (t, J=8 Hz, 2H), 7.09 (t, J=7.5 Hz, 1H), 6.91 (d, J=8 Hz, 2H), 6.77 (d, J=8 Hz, 1H), 5.21 (q, J=4.8 Hz, 1H), 4.74-4.68 (m, 1H), 4.64-4.53 (m, 2H), 4.24 (d, J=16 Hz, 1H), 4.12-4.01 (m, 2H), 3.88 (dd, J=44, 36 Hz, 1H), 2.65-2.47 (m, 1H), 2.26 (s, 3H), 2.15 (ddd, J=36.12 Hz, 1H), 1.56 (dd, J=28.8 Hz, 3H).LC/MS (ESI) m/z: 584 (M+H)⁺.

### Scheme 33: (2S,4R)—N—((R)-1-(5-carbamimidoylthiophen-2-yl)ethyl)-1-((9,9-difluoro-9H-fluorene-3-carbonyl)glycyl)-4-fluoro-4-(fluoromethyl)pyrrolidine-2-carboxamide (Compound 33)

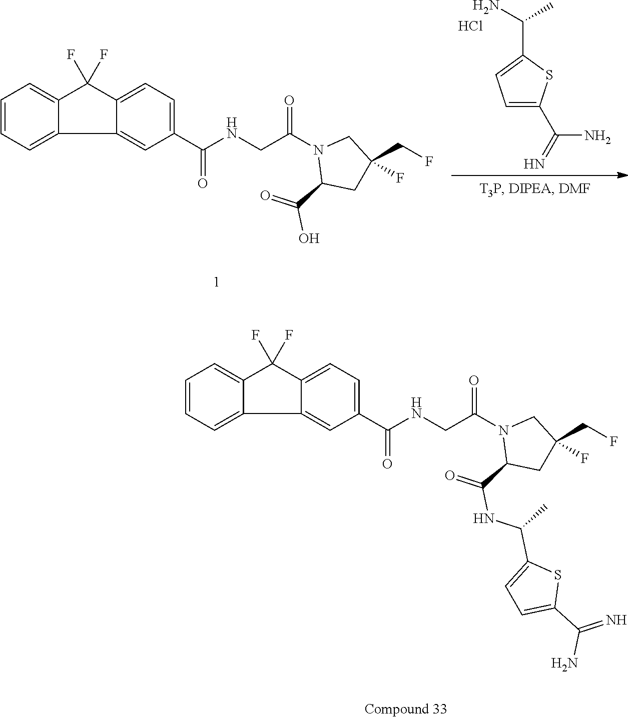 [SMILES: O=C(NCC(=O)N1C[C@@](F)(CF)C[C@H]1C(=O)O)c1ccc2c(c1)-c1ccccc1C2(F)F ; Cl ; C[C@@H](N)c1ccc(C(=N)N)s1 ; C[C@@H](NC(=O)[C@@H]1C[C@](F)(CF)CN1C(=O)CNC(=O)c1ccc2c(c1)-c1ccccc1C2(F)F)c1ccc(C(=N)N)s1]

Compound 33 was prepared as a white solid from (2S,4R)-1-((9,9-difluoro-9H-fluorene-3-carbonyl)glycyl)-4-fluoro-4-(fluoromethyl)pyrrolidine-2-carboxylic acid and (R)-5-(1-aminoethyl) thiophene-2-carboximidamide based on the procedures set forth in Scheme 32. ¹H-NMR (400 MHz, CD₃OD) δ 8.54 (s, 2H), 8.20 (s, 1H), 7.90 (d, J=7.7 Hz, 1H), 7.79-7.70 (m, 3H), 7.65 (d, J=7.0 Hz, 1H), 7.58 (t, J=7.3 Hz, 1H), 7.45 (t, J=7.4 Hz, 1H), 7.34-7.17 (m, 1H), 5.33-5.29 (m, 1H), 4.81 (s, 1H), 4.70-4.61 (m, 2H), 4.40 (d, J=16.9 Hz, 1H), 4.20-4.10 (m, 2H), 3.96 (dd, J=32.6, 11.5 Hz, 1H), 2.70-2.55 (m, 1H), 2.32-2.14 (m, 1H), 1.60 (d, J=6.9 Hz, 3H). LC/MS (ESI) m/z: 602 (M+H)⁺.

### Scheme 34: (2S,4R)—N—((R)-1-(4-carbamimidoylthiophen-2-yl)ethyl)-4-fluoro-4-(methoxymethyl)-1-((3-methyl-4-phenoxybenzoyl)glycyl)pyrrolidine-2-carboxamide (Compound 34)

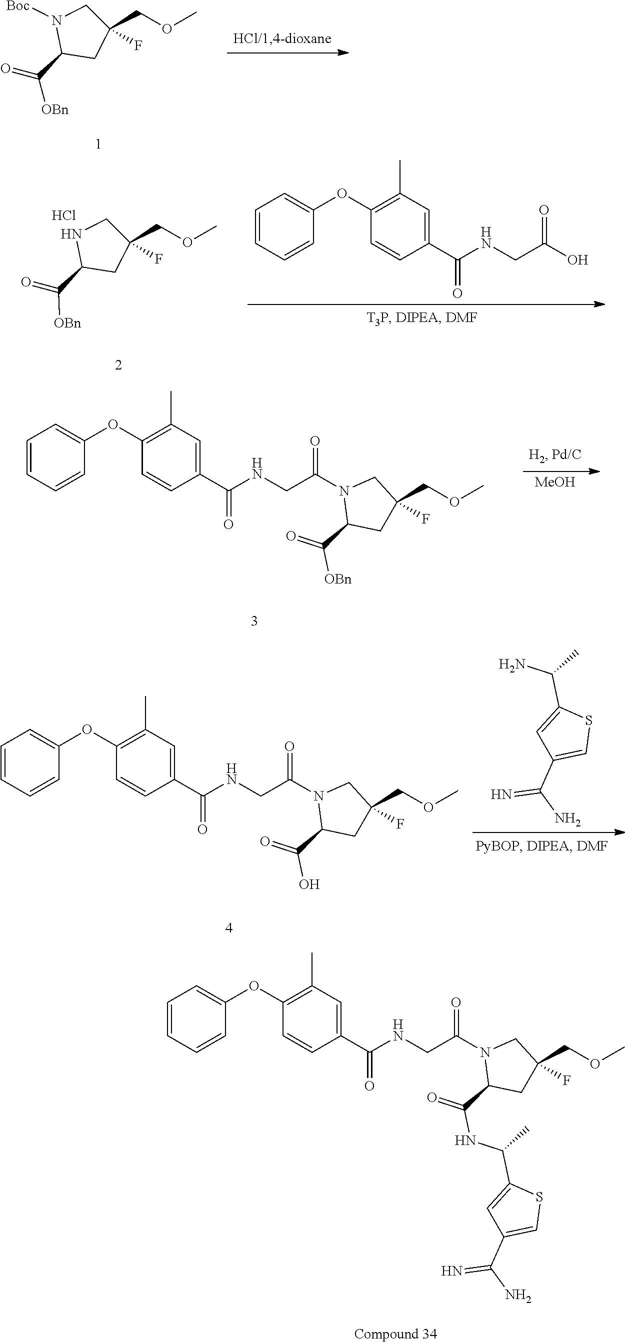

### Step 1: benzyl (2S,4R)-4-fluoro-4-(methoxymethyl)pyrrolidine-2-carboxylate hydrochloride (2)

A solution of 2-benzyl 1-(tert-butyl) (2S,4R)-4-fluoro-4-(methoxymethyl)pyrrolidine-1,2-dicarboxylate (180 mg, 0.49 mmol) in HCl/1,4-dioxane (2 mL, 4M) was stirred at 25° C. for 4 hours. The reaction mixture was concentrated to dryness, co-evaporated with DCM and dried under vacuum to give benzyl (2S,4R)-4-fluoro-4-(methoxymethyl)pyrrolidine-2-carboxylate hydrochloride (160 mg, yield 100%) as a white solid, which was directly used in the next reaction. LC/MS (ESI) m/z: 268 (M+H)⁺.

### Step 2: benzyl (2S,4R)-4-fluoro-4-(methoxymethyl)-1-((3-methyl-4-phenoxybenzoyl)glycyl) pyrrolidine-2-carboxylate (3)

At 0° C., to a mixture of benzyl (2S,4R)-4-fluoro-4-(methoxymethyl)pyrrolidine-2-carboxylate (33 mg, 0.11 mmol) and (3-methyl-4-phenoxybenzoyl)glycine (32 mg, 0.11 mmol) in DMF (2 mL) was added T₃P (210 mg, 0.33 mmol, 50% wt. in EtOAc) and DIPEA (0.1 mL, 0.66 mmol) under N₂ atmosphere and the mixture was stirred at room temperature overnight. The mixture was diluted with EtOAc and washed with saturated aq·NaHCO₃ solution. The aqueous layer was extracted with EtOAc twice and the combined organic layers were washed with brine, dried over anhydrous Na₂SO₄, filtered, and concentrated under reduced pressure to dryness. The residue was purified by flash chromatography on silica gel (DCM:MeOH=20:1) to afford benzyl (2S,4R)-4-fluoro-4-(methoxymethyl)-1-((3-methyl-4-phenoxybenzoyl)glycyl)pyrrolidine-2-carboxylate (50 mg, yield 86.2%) as a yellow solid. LC/MS (ESI) m/z: 535 (M+H)⁺

### Step 3: (2S,4R)-4-fluoro-4-(methoxymethyl)-1-((3-methyl-4-phenoxybenzoyl)glycyl) pyrrolidine-2-carboxylic acid

To a solution of benzyl (2S,4R)-4-fluoro-4-(methoxymethyl)-1-((3-methyl-4-phenoxybenzoyl)glycyl)pyrrolidine-2-carboxylate (50 mg, 0.09 mmol) in MeOH (2 mL) were added Pd/C (5 mg, 10% wt.) under N₂ atmosphere, the mixture was degassed under N₂ atmosphere for three times and stirred under a H₂ balloon at room temperature for 3 hours. The mixture was filtered, and the filtrate was concentrated under reduced pressure to dryness to give (2S,4R)-4-fluoro-4-(methoxymethyl)-1-((3-methyl-4-phenoxybenzoyl)glycyl) pyrrolidine-2-carboxylic acid (40 mg, yield 100%) as a yellow oil, which was used directly in the next reaction. LC/MS (ESI) m/z: 445 (M+H)⁺.

### Step 4: (2S,4R)—N—((R)-1-(4-carbamimidoylthiophen-2-yl)ethyl)-4-fluoro-4-(methoxymethyl)-1-((3-methyl-4-phenoxybenzoyl)glycyl)pyrrolidine-2-carboxamide (Compound 34)

At 0° C., to a mixture of (2S,4R)-4-fluoro-4-(methoxymethyl)-1-((3-methyl-4-phenoxybenzoyl)glycyl)pyrrolidine-2-carboxylic acid (40 mg, 0.09 mmol) and (R)-5-(1-aminoethyl)thiophene-3-carboximidamide (18 mg, 0.11 mmol) in DMF (2 mL) was added PyBOP (70 mg, 0.14 mmol) and DIPEA (0.1 mL, 0.45 mmol) under N₂ atmosphere and the mixture was stirred at room temperature for half an hour. The mixture was diluted with EtOAc, washed with water and brine, dried over anhydrous Na₂SO₄, filtered, and concentrated under reduced pressure to dryness. The residue was purified by prep-HPLC to afford (2S,4R)—N—((R)-1-(4-carbamimidoylthiophen-2-yl)ethyl)-4-fluoro-4-(methoxymethyl)-1-((3-methyl-4-phenoxybenzoyl)glycyl)pyrrolidine-2-carboxamide (3.0 mg, yield 5.7%) as a white solid. ¹HNMR (400 MHz, CD₃OD) δ 8.43 (s, 1H), 8.23 (dd, J=10.0, 10.0 Hz, 1H), 7.79 (dd, J=9.2, 9.6 Hz, 1H), 7.65 (dd, J=8.4, 8.8 Hz, 1H), 7.56-7.51 (m, 1H), 7.37 (t, J=8.0 Hz, 2H), 7.14 (t, J=7.4 Hz, 1H), 6.96 (d, J=8.0 Hz, 2H), 6.82 (d, J=8.4 Hz, 1H), 5.37-5.22 (m, 1H), 4.65-4.56 (m, 1H), 4.29-4.08 (m, 2H), 4.07-3.98 (m, 1H), 3.94-3.83 (m, 1H), 3.74-3.65 (m, 2H), 3.43 (s, 3H), 2.61-2.43 (m, 1H), 2.31 (s, 3H), 2.27-2.10 (m, 1H), 1.60 (t, J=14.9 Hz, 3H). LC/MS (ESI) m/z: 596 (M+H)⁺.

### Scheme 35: Synthesis of (2S,4S)-4-(aminomethyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (Compound 35)

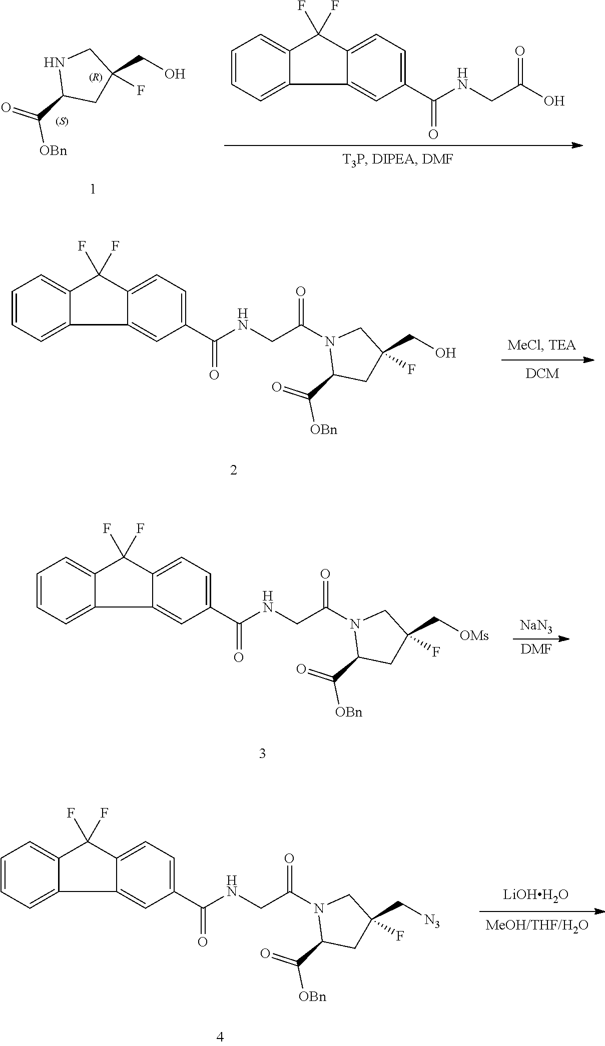

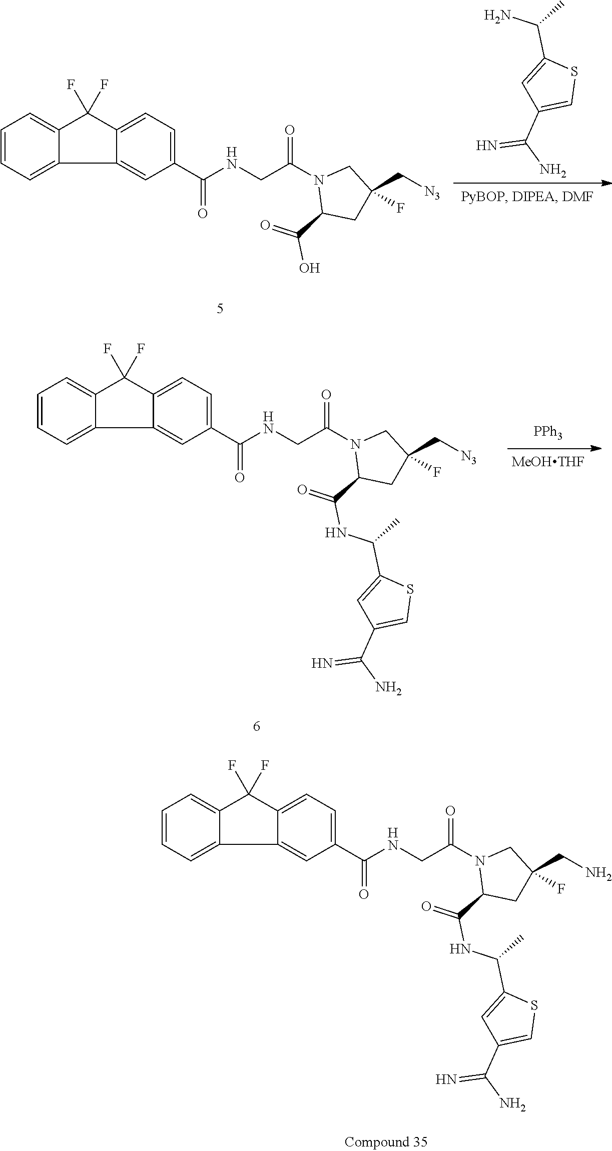 [SMILES: [N-]=[N+]=NC[C@@]1(F)C[C@@H](C(=O)O)N(C(=O)CNC(=O)c2ccc3c(c2)-c2ccccc2C3(F)F)C1 ; C[C@@H](N)c1cc(C(=N)N)cs1 ; C[C@@H](NC(=O)[C@@H]1C[C@](F)(CN=[N+]=[N-])CN1C(=O)CNC(=O)c1ccc2c(c1)-c1ccccc1C2(F)F)c1cc(C(=N)N)cs1 ; C[C@@H](NC(=O)[C@@H]1C[C@](F)(CN)CN1C(=O)CNC(=O)c1ccc2c(c1)-c1ccccc1C2(F)F)c1cc(C(=N)N)cs1 ; "PPh3"]

### Step 1: benzyl (2S,4R)-1-((9,9-difluoro-9H-fluorene-3-carbonyl)glycyl)-4-fluoro-4-(hydroxymethyl)pyrrolidine-2-carboxylate (2)

To a mixture of benzyl (2S,4R)-4-fluoro-4-(fluoromethyl)pyrrolidine-2-carboxylate (96 mg, 0.38 mmol) and (9,9-difluoro-9H-fluorene-3-carbonyl)glycine (115 mg, 0.38 mmol) in DMF (3 mL) was added DIPEA (0.40 mL, 2.28 mmol) and T₃P (725 mg, 1.14 mmol, 50% wt. in EtOAc) at 0° C. under N₂ atmosphere and the mixture was stirred at 30° C. for 1 hour. The mixture was diluted with EtOAc and washed with saturated NaHCO₃ solution. The aqueous layer was extracted with EtOAc twice. The combined organic layers were washed with brine, dried over anhydrous Na₂SO₄, filtered, and concentrated to dryness under reduced pressure. The residue was purified by flash chromatography on silica gel (DCM:MeOH=10:1) to give benzyl (2S,4R)-1-((9,9-difluoro-9H-fluorene-3-carbonyl)glycyl)-4-fluoro-4-(hydroxymethyl)pyrrolidine-2-carboxylate (160 mg, yield 78.4%) as a yellow solid. LC/MS (ESI) (m/z): 539 (M+H)⁺.

### Step 2: benzyl (2S,4R)-1-((9,9-difluoro-9H-fluorene-3-carbonyl)glycyl)-4-fluoro-4-(((methylsulfonyl)oxy)methyl)pyrrolidine-2-carboxylate (3)

To a solution of benzyl (2S,4R)-1-((9,9-difluoro-9H-fluorene-3-carbonyl)glycyl)-4-fluoro-4-(hydroxymethyl)pyrrolidine-2-carboxylate (160 mg, 0.30 mmol) in DCM (5 mL) was added TEA (0.3 mL, 2.08 mmol) and MsCl (103 mg, 0.90 mmol) at 0° C. under N₂ atmosphere. The reaction mixture was stirred at 0° C. for 1.5 hours. The mixture was quenched with ice-water and extracted with DCM twice. The combined organic layers were washed with brine, dried over anhydrous Na₂SO₄, filtered, and concentrated under reduced pressure to dryness to give benzyl (2S,4R)-1-((9,9-difluoro-9H-fluorene-3-carbonyl)glycyl)-4-fluoro-4-(((methylsulfonyl) oxy)methyl)pyrrolidine-2-carboxylate (180 mg, yield 98.4%) as a yellow solid, which was used directly in the next reaction without further purification. LC/MS (ESI) m/z: 617 (M+H)⁺.

### Step 3: benzyl (2S,4R)-4-(azidomethyl)-1-((9,9-difluoro-9H-fluorene-3-carbonyl)glycyl)-4-fluoropyrrolidine-2-carboxylate (4)

To a solution of benzyl (2S,4R)-1-((9,9-difluoro-9H-fluorene-3-carbonyl)glycyl)-4-fluoro-4-(((methylsulfonyl)oxy)methyl)pyrrolidine-2-carboxylate (180 mg, 0.29 mmol) in DMF (5 mL) was added NaN₃ (58 mg, 0.88 mmol) and TBAI (11 mg, 0.03 mmol) and the mixture was stirred at 65° C. overnight. The mixture was diluted with EtOAc, washed with water and brine, dried over anhydrous Na₂SO₄, filtered, and concentrated under reduced pressure to dryness. The residue was purified by column chromatography on silica gel (PE:EtOAc=1:1) to give benzyl (2S,4R)-4-(azidomethyl)-1-((9,9-difluoro-9H-fluorene-3-carbonyl)glycyl)-4-fluoropyrrolidine-2-carboxylate (90 mg, yield 54.9%) as a yellow oil. LC/MS (ESI) m/z: 564 (M+H)⁺.

### Step 4: (2S,4R)-4-(azidomethyl)-1-((9,9-difluoro-9H-fluorene-3-carbonyl)glycyl)-4-fluoropyrrolidine-2-carboxylic acid (5)

To a solution of benzyl (2S,4R)-4-(azidomethyl)-1-((9,9-difluoro-9H-fluorene-3-carbonyl)glycyl)-4-fluoropyrrolidine-2-carboxylate (90 mg, 0.16 mmol) in MeOH (2 mL) and THF (1 mL) was added a solution of LiOH (14 mg, 0.32 mmol) in water (1 mL) at 0° C., and the mixture was stirred at room temperature for 2 hours. The mixture was diluted with water and washed with EtOAc twice. The aqueous layer was acidified with 0.5 M aq·HCl to pH˜4 and extracted with EtOAc twice. The combined organic layers were washed with brine, dried over anhydrous Na₂SO₄, filtered, and concentrated to dryness to give (2S,4R)-4-(azidomethyl)-1-((9,9-difluoro-9H-fluorene-3-carbonyl)glycyl)-4-fluoropyrrolidine-2-carboxylic acid (50 mg, yield 66.1%) as a light-yellow solid, which was used directly in the next reaction without further purification. LC/MS (ESI) m/z: 474 (M+H)⁺.

### Step 5: (2S,4R)-4-(azidomethyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (6)

To a mixture of (2S,4R)-4-(azidomethyl)-1-((9,9-difluoro-9H-fluorene-3-carbonyl)glycyl)-4-fluoropyrrolidine-2-carboxylic acid (50 mg, 0.11 mmol) and (R)-5-(1-aminoethyl)thiophene-3-carboximidamide (27 mg, 0.16 mmol) in DMF (3 mL) was added DIPEA (0.087 mL, 0.53 mmol) and PyBOP (83 mg, 0.16 mmol) at 0° C. under N₂ atmosphere and the mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography on silica gel (DCM:MeOH=20:1) to give (2S,4R)-4-(azidomethyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (40 mg, yield 60.6%) as a white solid. LC/MS (ESI) (m/z): 625 (M+H)⁺.

### Step 6: (2S,4S)-4-(aminomethyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (Compound 35)

To a solution of (2S,4R)-4-(azidomethyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (30 mg, 0.048 mmol) in THF (2 mL) and water (2 mL) was added PPh₃ (50 mg, 0.19 mmol) and the mixture was stirred at 45° C. for 16 hours. The mixture was acidified with 0.5 M HCl solution to pH˜4 and washed with EtOAc twice. The aqueous layer was basified with saturated NaHCO₃ solution to pH˜8 and extracted with CHCl₃/i-PrOH(3/1, v/v) twice. The combined organic layers were washed with brine, dried over anhydrous Na₂SO₄, filtered, and concentrated to dryness. The residue was purified by prep-HPLC to give Compound 35 (2.6 mg, yield 9.1%) as a white solid. ¹H NMR (400 MHz, CD₃OD) δ 8.28-8.13 (m, 2H), 7.91-7.84 (m, 1H), 7.74 (dd, J=15.2, 15.2 Hz, 2H), 7.65 (d, J=7.2 Hz, 1H), 7.62-7.52 (m, 2H), 7.45 (t, J=7.4 Hz, 1H), 5.54-5.22 (m, 1H), 4.82-4.74 (m, 1H), 4.71-4.57 (m, 1H), 4.42-4.27 (m, 1H), 4.26-4.06 (m, 2H), 3.97-3.83 (m, 1H), 3.49-3.35 (m, 1H), 2.93-2.61 (m, 1H), 2.35-2.10 (m, 1H), 1.63 (dd, J=7.2, 7.2 Hz, 3H). LC/MS (ESI) m/z: 599 (M+H)⁺.

### Scheme 36: Synthesis of (2S,4S)—N—((R)-1-(4-carbamimidoylthiophen-2-yl)ethyl)-1-((9,9-difluoro-9H-fluorene-3-carbonyl)glycyl)-4-((dimethylamino)methyl)-4-fluoropyrrolidine-2-carboxamide (Compound 36)

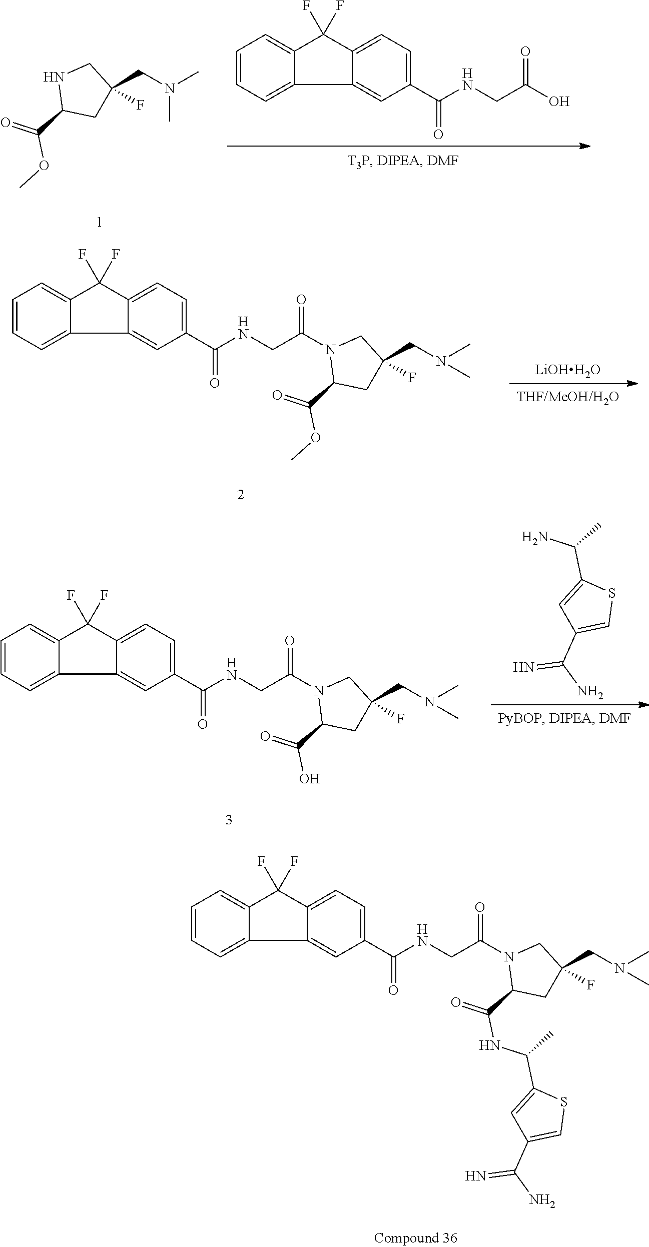

Compound 36 was prepared as a white solid from methyl (2S,4R)-4-((dimethylamino)methyl)-4-fluoropyrrolidine-2-carboxylate, (methyl (2S,4S)-1-((9,9-difluoro-9H-fluorene-3-carbonyl)glycyl)-4-((dimethylamino)methyl)-4-fluoropyrrolidine-2-carboxylate, and (R)-5-(1-aminoethyl)thiophene-3-carboximidamide based on the procedures set forth in Scheme 7. ¹H NMR (400 MHz, CD₃OD) δ 8.27-8.15 (m, 2H), 7.91-7.83 (m, 1H), 7.81-7.69 (m, 2H), 7.65 (d, J=7.2 Hz, 1H), 7.62-7.51 (m, 2H), 7.45 (t, J=7.4 Hz, 1H), 5.42-5.20 (m, 1H), 4.72-4.58 (m, 1H), 4.46-4.26 (m, 1H), 4.22-4.06 (m, 2H), 3.94-3.80 (m, 1H), 2.87-2.78 (m, 2H), 2.71-2.53 (m, 1H), 2.38 (d, J=3.8 Hz, 6H), 2.23-2.03 (m, 1H), 1.63 (dd, J=6.8, 6.8 Hz, 3H). LC/MS (ESI) m/z: 627 (M+H)⁺.

### Scheme 37: Synthesis of (2S,4R)—N—((R)-1-(4-carbamimidoylthiophen-2-yl)ethyl)-1-((4-(4-fluorophenoxy)benzoyl)glycyl)-4-phenoxypyrrolidine-2-carboxamide (Compound 37)

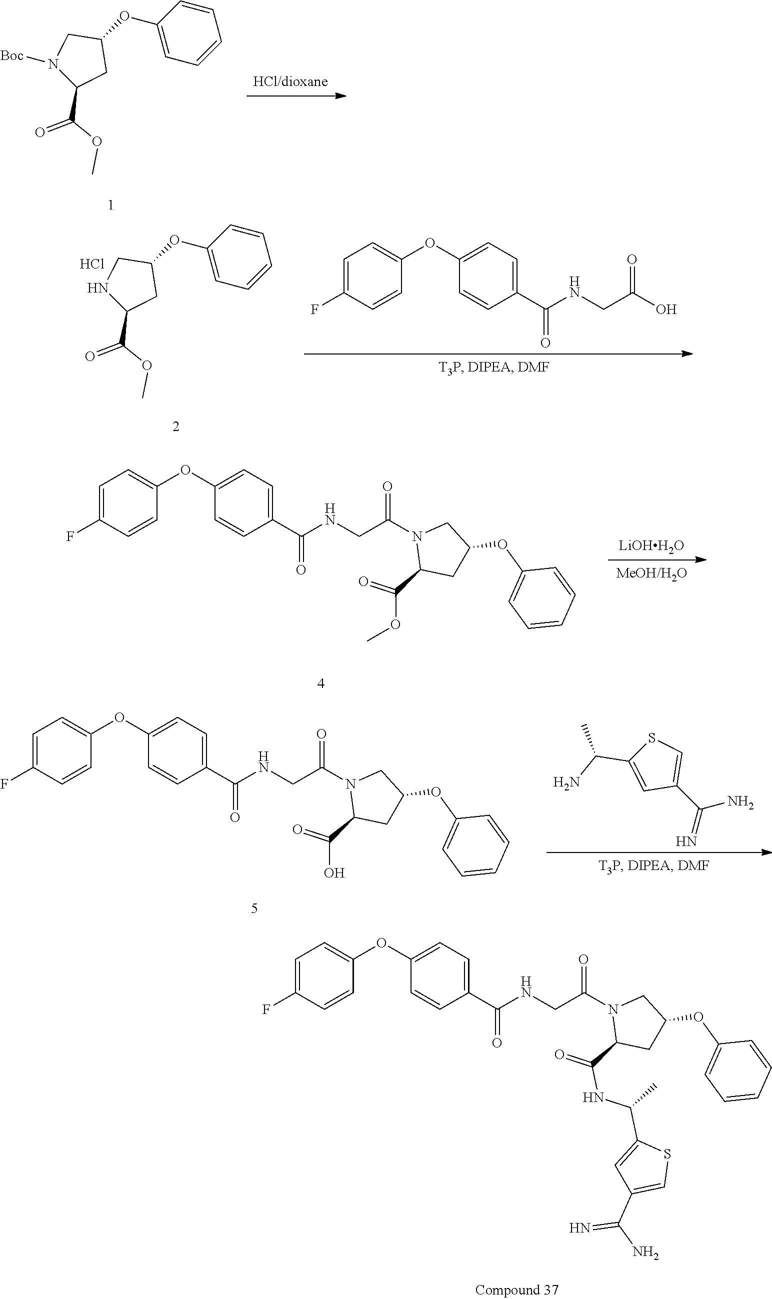

### Step 1: methyl (2S,4R)-4-phenoxypyrrolidine-2-carboxylate hydrochloride (2)

A mixture of 1-(tert-butyl) 2-methyl (2S,4R)-4-phenoxypyrrolidine-1,2-dicarboxylate (120 mg, 0.37 mmol) in HCl/1,4-dioxane (3 mL, 4M) was stirred at room temperature for 1 hour. The reaction mixture was concentrated to dryness under reduced pressure, co-evaporated with DCM and dried under vacuum to give methyl (2S,4R)-4-phenoxypyrrolidine-2-carboxylate hydrochloride (95 mg, yield 100%) as a light-yellow solid, which was used directly in the next reaction without further purification. LC/MS (ESI) m/z: 222 (M+H)⁺.

### Step 2: tert-butyl (S)-2-(hydroxymethyl)-4-methylenepyrrolidine-1-carboxylate (4)

To a mixture of methyl (2S,4R)-4-phenoxypyrrolidine-2-carboxylate hydrochloride (95 mg, 0.37 mmol) and (4-(4-fluorophenoxy)benzoyl)glycine (108 mg, 0.37 mmol) in DMF (5 mL) was added DIPEA (0.3 mL, 1.85 mmol) and T₃P (1713 mg, 1.11 mmol, 50% wt. in EtOAc) at 0° C. under N₂ atmosphere and the mixture was stirred at 25° C. for 2 hours. The mixture was diluted with EtOAc, washed with water and brine, dried over anhydrous Na₂SO₄, filtered, and concentrated under reduced pressure to dryness. The residue was purified by flash chromatography (PE:EtOAc=1:1) to give methyl (2S,4R)-1-((4-(4-fluorophenoxy)benzoyl)glycyl)-4-phenoxypyrrolidine-2-carboxylate (138 mg, yield 75.4%) as a yellow solid. LC/MS (ESI) m/z: 493 (M+H)⁺.

### Step 3: tert-butyl (S)-2-(((tert-butyldimethylsilyl)oxy)methyl)-4-methylenepyrrolidine-1-carboxylate (5)

To a solution of methyl (2S,4R)-1-((4-(4-fluorophenoxy)benzoyl)glycyl)-4-phenoxypyrrolidine-2-carboxylate (138 mg, 0.28 mmol) in MeOH (3 mL) was added a solution of LiOH (18 mg, 0.42 mmol) in water (1 mL) at 0° C. and the mixture was stirred at room temperature for 2 hours. The mixture was diluted with water and washed with EtOAc twice. The aqueous layer was acidified with 0.5N aq·HCl to pH˜4 and extracted with EtOAc twice. The combined organic layers were washed with brine, dried over anhydrous Na₂SO₄, filtered, and concentrated to dryness to give (2S,4R)-1-((4-(4-fluorophenoxy)benzoyl)glycyl)-4-phenoxypyrrolidine-2-carboxylic acid (130 mg, yield 97%) as a white solid, which was used directly in the next reaction without further purification. LC/MS (ESI) m/z: 479 (M+H)⁺.

### Step 4: (2S,4R)—N—((R)-1-(4-carbamimidoylthiophen-2-yl)ethyl)-1-((4-(4-fluorophenoxy) benzoyl)glycyl)-4-phenoxypyrrolidine-2-carboxamide (Compound 37)

To a mixture of (2S,4R)-1-((4-(4-fluorophenoxy)benzoyl)glycyl)-4-phenoxypyrrolidine-2-carboxylic acid (80 mg, 0.17 mmol) and (R)-5-(1-aminoethyl)thiophene-3-carboximidamide (57 mg, 0.34 mmol) in DMF (3 mL) was added DIPEA (0.14 mL, 0.85 mmol) and T₃P (128 mg, 0.20 mmol, 50% wt. in EtOAc) at 0° C. under N₂ atmosphere and the mixture was stirred at 25° C. for 3 hours. The mixture was quenched with saturated NaHCO₃ solution and extracted with CHCl₃/i-PrOH (3/1, v/v) twice. The combined organic layers were washed with brine, dried over anhydrous Na₂SO₄, filtered, and concentrated under reduced pressure to dryness. The residue was purified by prep-TLC (DCM:MeOH=10:1) and further purified by prep-HPLC to give Compound 37 (20 mg, yield 19%) as a white solid. ¹HNMR (400 MHz, CD₃OD) δ 8.50 (s, 1H), 7.86-7.82 (m, 2H), 7.42 (dd, J=8.5, 7.5 Hz, 2H), 7.21 (t, J=7.4 Hz, 1H), 7.08-7.05 (m, 2H), 7.02-6.99 (m, 2H), 4.71 (s, 2H), 4.33 (d, J=7.3 Hz, 2H), 3.41 (dd, J=6.0, 2.4 Hz, 1H), 2.44 (t, J=11.8 Hz, 1H), 2.22 (dd, J=13.4, 3.5 Hz, 1H), 1.31 (s, 3H), 1.23 (dd, J=5.8, 2.5 Hz, 1H), 0.82 (t, J=5.3 Hz, 1H). LC/MS (ESI) (m/z): 630 (M+H)⁺.

### Scheme 38: Synthesis of (1S,3S,5R)—N—((R)-1-(5-carbamimidoylthiophen-2-yl)ethyl)-2-((4-(4-fluorophenoxy)benzoyl)glycyl)-5-(methoxymethyl)-2-azabicyclo[3.1.0]-hexane-3-carboxamide (Compound 38)

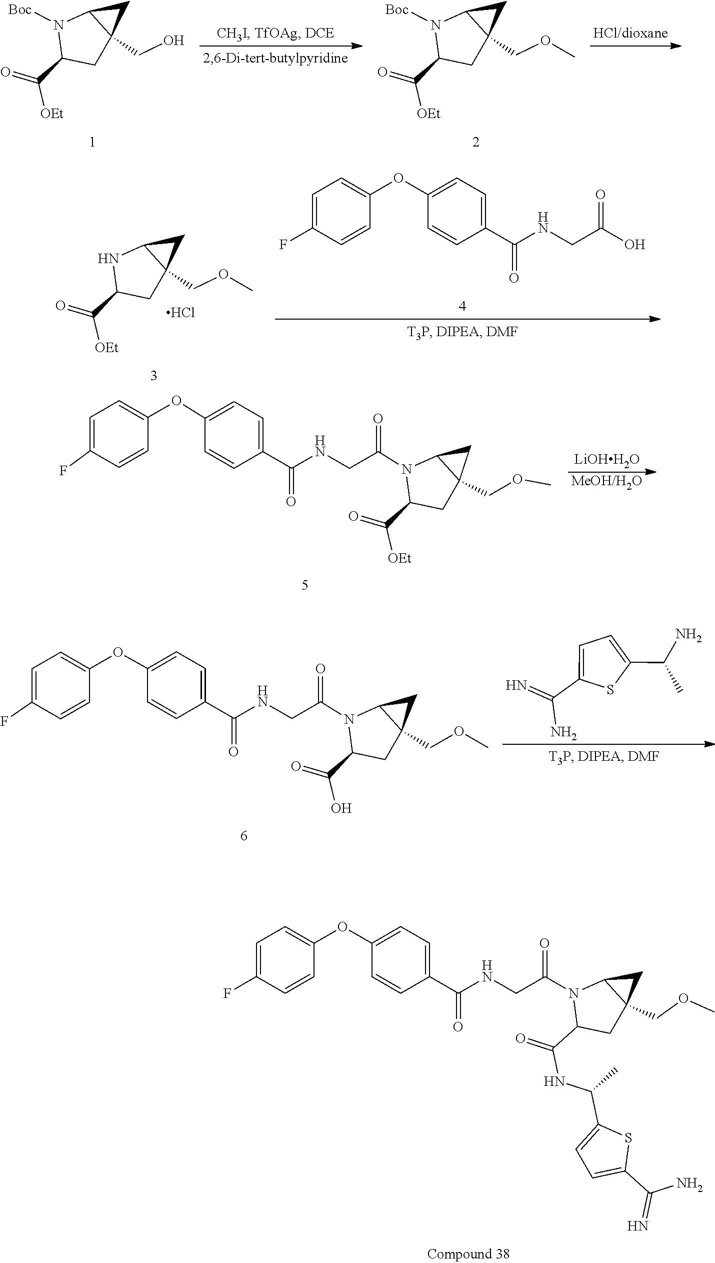

### Step 1: 2-(tert-butyl) 3-ethyl (1S,3S,5R)-5-(methoxymethyl)-2-azabicyclo[3.1.0]hexane-2,3-dicarboxylate (2)

To a solution of 2-(tert-butyl) 3-ethyl-(1 S,3S,5R)-5-(hydroxymethyl)-2-azabicyclo[3.1.0]hexane-2,3-dicarboxylate (300 mg, 1.05 mmol) in DCE (5 mL) was added AgOTf (1.3 g, 5.25 mmol) followed by methyl iodide (747 mg, 5.25 mmol) and 2,6-di-tert-butylpyridine (1.2 mL, 5.25 mmol) and the mixture was stirred at 30° C. for 16 hours under N₂ atmosphere. The mixture was filtered, and the filtrate was concentrated to dryness. The residue was purified by flash chromatography on silica gel (PE:EtOAc=3:1) to give 2-(tert-butyl) 3-ethyl (1S,3S,5R)-5-(methoxymethyl)-2-azabicyclo[3.1.0]hexane-2,3-dicarboxylate (136 mg, yield 43.2%) as a colorless oil. LC/MS (ESI) (m/z): 300 (M+H)⁺.

### Step 2: 2-benzyl 1-(tert-butyl) (2S,4R)-4-((((E)-6-ethoxy-6-oxohex-2-en-1-yl)oxy)methyl)-4-fluoropyrrolidine-1,2-dicarboxylate (3)

A solution of 2-(tert-butyl) 3-ethyl-(1S,3S,5R)-5-(methoxymethyl)-2-azabicyclo[3.1.0]hexane-2,3-dicarboxylate (136 mg, 0.45 mmol) in HCl/1,4-dioxane (5 mL, 4 M) was stirred at room temperature for 1 hour. The reaction mixture was concentrated to dryness under reduced pressure, co-evaporated with DCM and dried under vacuum to give ethyl (1S,3S,5R)-5-(methoxymethyl)-2-azabicyclo[3.1.0]hexane-3-carboxylate hydrochloride (90 mg, yield 100%) as a brown oil, which was used directly in the next step without further purification. LC/MS (ESI) m/z: 200 (M+H)⁺.

### Step 3: ethyl (1S,3S,5R)-2-((4-(4-fluorophenoxy)benzoyl)glycyl)-5-(methoxymethyl)-2-azabicyclo[3.1.0]hexane-3-carboxylate (5)

To a mixture of ethyl (1S,3S,5R)-5-(methoxymethyl)-2-azabicyclo[3.1.0]hexane-3-carboxylate (90 mg, 0.45 mmol) and (4-(4-fluorophenoxy)benzoyl)glycine (131 mg, 0.45 mmol) in DMF (5.0 mL) was added DIPEA (0.37 mL, 2.25 mmol) and T₃P (862 mg, 1.35 mmol, 50% wt. in EtOAc) at 0° C. under N₂ atmosphere and the mixture was stirred at 25° C. for 2 hours. The mixture was diluted with EtOAc, washed with water and brine, dried over anhydrous Na₂SO₄, filtered, and concentrated under reduced pressure to dryness. The residue was purified by flash chromatography on silica gel (DCM:MeOH=20:1) to give ethyl (1 S,3S,5R)-2-((4-(4-fluorophenoxy)benzoyl)glycyl)-5-(methoxymethyl)-2-azabicyclo[3.1.0]hexane-3-carboxylate (190 mg, yield 89.6%) as a brown oil. LC/MS (ESI) m/z: 471 (M+H)⁺.

### Step 4: (1S,3S,5R)-2-((4-(4-fluorophenoxy)benzoyl)glycyl)-5-(methoxymethyl)-2-azabicyclo [3.1.0]hexane-3-carboxylic acid (6)

To a solution of ethyl (1 S,3S,5R)-2-((4-(4-fluorophenoxy)benzoyl)glycyl)-5-(methoxymethyl)-2-azabicyclo[3.1.0]hexane-3-carboxylate (190 mg, 0.40 mmol) in MeOH (3 mL) was added a solution of LiOH (25.5 mg, 0.61 mmol) in water (1 mL) at 0° C., and the mixture was stirred at room temperature for 2 hours. The mixture was diluted with water and washed with EtOAc twice. The aqueous layer was acidified with 0.5N aq·HCl to pH˜4 and extracted with EtOAc twice. The combined organic layers were washed with brine, dried over anhydrous Na₂SO₄, filtered, and concentrated to dryness to give (1S,3S,5R)-2-((4-(4-fluorophenoxy)benzoyl)glycyl)-5-(methoxymethyl)-2-azabicyclo[3.1.0]hexane-3-carboxylic acid (173 mg, yield 97.2%) as a yellow solid, which was used directly in the next reaction without further purification. LC/MS (ESI) m/z: 443 (M+H)⁺.

### Step 5: (1S,3S,5R)—N—((R)-1-(5-carbamimidoylthiophen-2-yl)ethyl)-2-((4-(4-fluorophenoxy) benzoyl)glycyl)-5-(methoxymethyl)-2-azabicyclo[3.1.0]hexane-3-carboxamide (Compound 38)

To a mixture of (1 S,3S,5R)-2-((4-(4-fluorophenoxy)benzoyl)glycyl)-5-(methoxymethyl)-2-azabicyclo[3.1.0]hexane-3-carboxylic acid (60 mg, 0.14 mmol) and (R)-5-(1-aminoethyl) thiophene-2-carboximidamide (46 mg, 0.28 mmol) in DMF (3 mL) was added DIPEA (0.11 mL, 0.70 mmol) and T₃P (104 mg, 0.17 mmol, 50% wt. in EtOAc) at 0° C. under N₂ atmosphere and the mixture was stirred at 25° C. for 3 hours. The mixture was quenched with saturated NaHCO₃ solution and extracted with CHCl₃/i-PrOH (3/1, v/v) twice. The combined organic layers were washed with brine, dried over anhydrous Na₂SO₄, filtered, and concentrated under reduced pressure to dryness. The residue was purified by prep-TLC (DCM:MeOH=10:1) and further purified by prep-HPLC to give Compound 38 (5 mg, yield 6.2%) as a white solid. ¹HNMR (400 MHz, CD₃OD) δ 8.50 (s, 1H), 7.86-7.82 (m, 2H), 7.42 (dd, J=8.5, 7.5 Hz, 2H), 7.21 (t, J=7.4 Hz, 1H), 7.08-7.05 (m, 2H), 7.02-6.99 (m, 2H), 4.71 (s, 2H), 4.33 (d, J=7.3 Hz, 2H), 3.41 (dd, J=6.0, 2.4 Hz, 1H), 2.44 (t, J=11.8 Hz, 1H), 2.22 (dd, J=13.4, 3.5 Hz, 1H), 1.31 (s, 3H), 1.23 (dd, J=5.8, 2.5 Hz, 1H), 0.82 (t, J=5.3 Hz, 1H). LC/MS (ESI) (m/z): 594 (M+H)⁺.

### Scheme 39: (2S,4R)—N—((R)-1-(4-carbamimidoylthiophen-2-yl)ethyl)-4-(difluoromethoxy)-1-((4-(4-ethylphenoxy)benzoyl)glycyl)pyrrolidine-2-carboxamide (Compound 39)

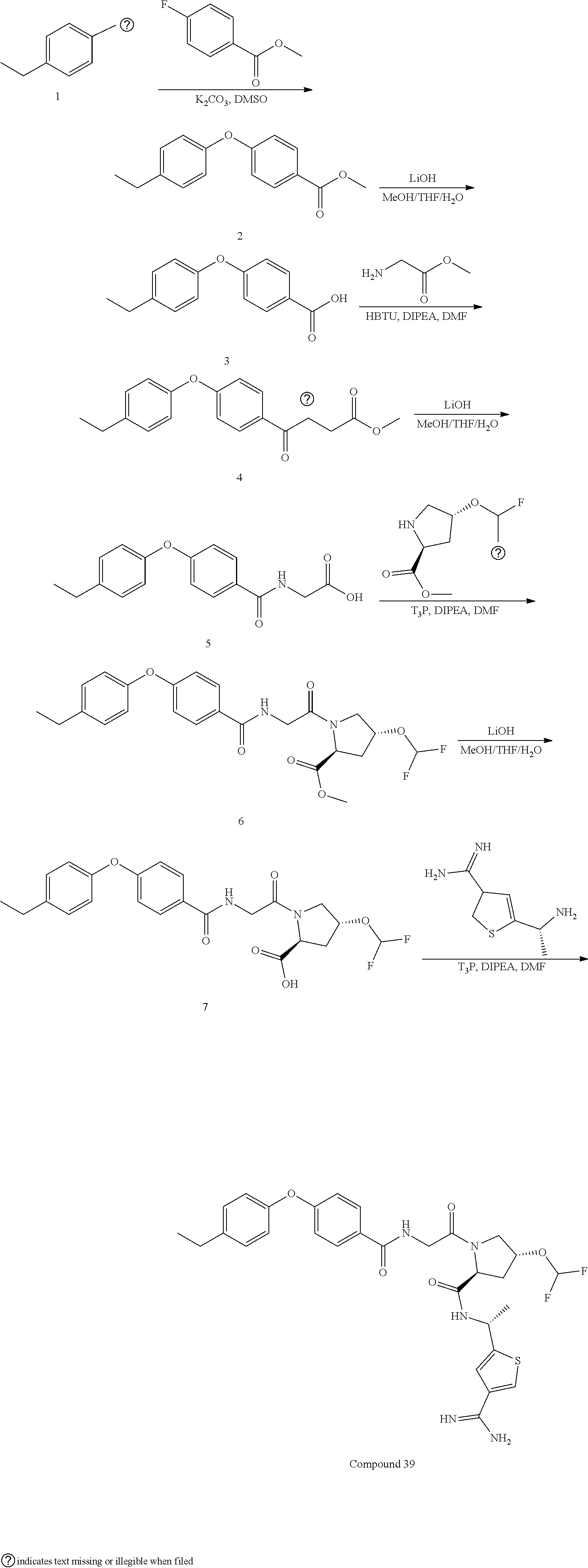

Compound 39 was prepared as a white solid from 4-ethylphenol, methyl 4-fluorobenzoate, methyl glycinate hydrochloride, methyl (2S,4R)-4-(difluoromethoxy)pyrrolidine-2-carboxylate, and (R)-5-(1-aminoethyl)thiophene-3-carboximidamide based on the procedures set forth in Scheme 24. ¹H NMR (400 MHz, CD₃OD) δ 8.54 (s, 1H), 8.22 (t, J=4.2 Hz, 1H), 7.86-7.80 (m, 2H), 7.54 (d, J=11.6 Hz, 1H), 7.25 (d, J=8.5 Hz, 2H), 6.99-6.95 (m, 4H), 5.29 (dt, J=20.3, 6.9 Hz, 1H), 4.57 (s, 1H), 4.17 (dd, J=32.1, 15.1 Hz, 2H), 3.98-3.86 (m, 2H), 2.65 (t, J=7.6 Hz, 2H), 2.51-2.41 (m, 1H), 2.26-2.19 (m, 1H), 1.60 (t, J=14.9 Hz, 3H), 1.24 (t, J=7.6 Hz, 3H). LC/MS (ESI) (m/z): 614 (M+H)⁺.

### Scheme 40: Synthesis of (2S,4R)—N—((R)-1-(5-carbamimidoylthiophen-2-yl)ethyl)-4-fluoro-4-(fluoromethyl)-1-((4-(4-fluorophenoxy)benzoyl)glycyl)pyrrolidine-2-carboxamide (Compound 40)

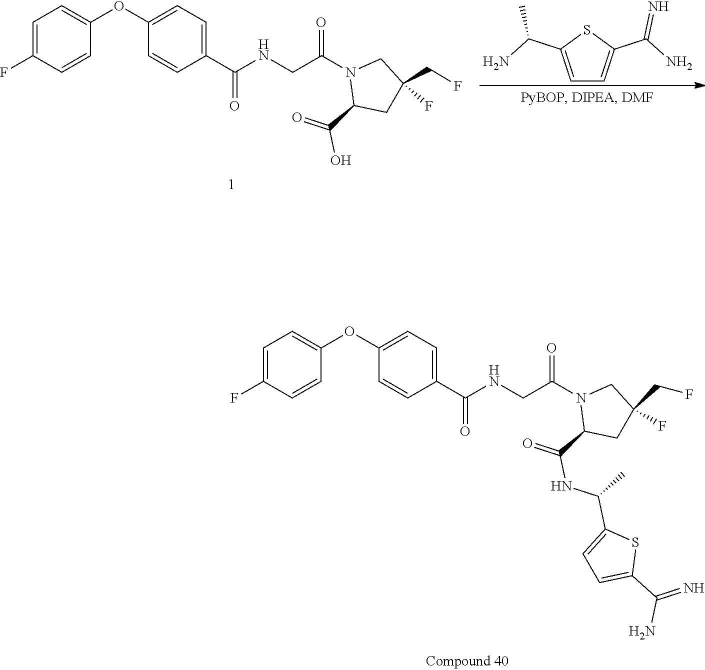 [SMILES: O=C(NCC(=O)N1C[C@@](F)(CF)C[C@H]1C(=O)O)c1ccc(Oc2ccc(F)cc2)cc1 ; C[C@@H](N)c1ccc(C(=N)N)s1 ; C[C@@H](NC(=O)[C@@H]1C[C@](F)(CF)CN1C(=O)CNC(=O)c1ccc(Oc2ccc(F)cc2)cc1)c1ccc(C(=N)N)s1]

Compound 40 was prepared as a white solid from (2S,4R)-4-fluoro-4-(fluoromethyl)-1-((4-(4-fluorophenoxy)benzoyl)glycyl) pyrrolidine-2-carboxylic acid and (R)-5-(1-aminoethyl)thiophene-2-carboximidamide based on the procedures set forth in Scheme 3. ¹H-NMR (400 MHz, CD₃OD) δ 8.49 (s, 1H), 7.88-7.85 (m, 2H), 7.83-7.78 (m, 1H), 7.27-7.23 (m, 1H), 7.19-7.14 (m, 2H), 7.11-7.07 (m, 2H), 7.01-6.98 (m, 2H), 5.51-5.15 (m, 1H), 4.80-4.75 (m, 1H), 4.68-4.62 (m, 2H), 4.37-4.30 (m, 1H), 4.14-4.07 (m, 2H), 3.99-3.88 (m, 1H), 2.65-2.54 (m, 1H), 2.28-2.17 (m, 1H), 1.66-1.58 (m, 3H). LC/MS (ESI) (m/z): 588 (M+H)⁺.

### Scheme 41: Synthesis of (1S,3S,5R)—N—((R)-1-(5-carbamimidoylthiophen-2-yl)ethyl)-2-((4-(4-fluorophenoxy)benzoyl)glycyl)-5-(hydroxymethyl)-2-azabicyclo[3.1.0]hexane-3-carboxamide (Compound 41)

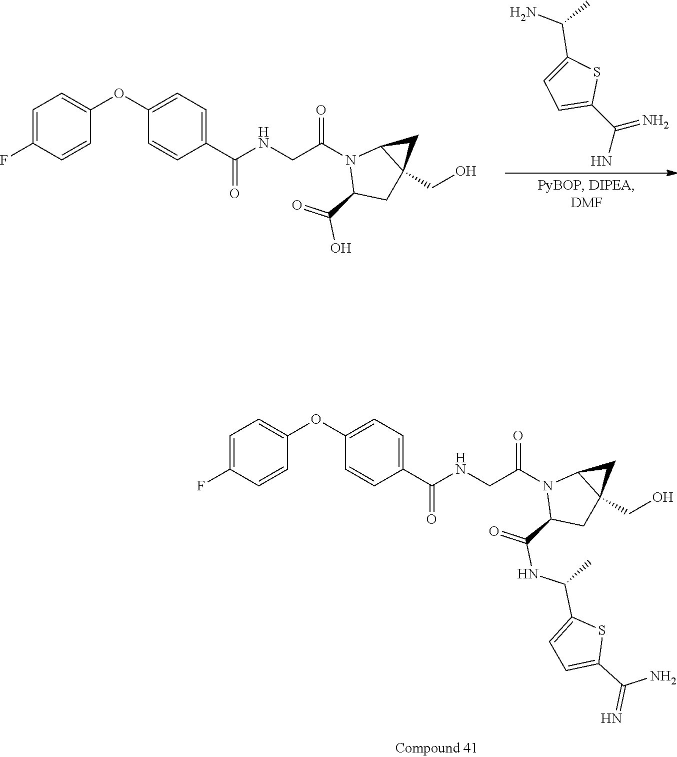 [SMILES: O=C(NCC(=O)N1[C@H]2C[C@@]2(CO)C[C@H]1C(=O)O)c1ccc(Oc2ccc(F)cc2)cc1 ; C[C@@H](N)c1ccc(C(=N)N)s1 ; C[C@@H](NC(=O)[C@@H]1C[C@]2(CO)C[C@@H]2N1C(=O)CNC(=O)c1ccc(Oc2ccc(F)cc2)cc1)c1ccc(C(=N)N)s1]

Compound 41 was prepared as a white solid from (1 S,3S,5R)-2-((4-(4-fluorophenoxy)benzoyl)glycyl)-5-(hydroxymethyl)-2-azabicyclo[3.1.0]hexane-3-carboxylic acid and (R)-5-(1-aminoethyl)thiophene-2-carboximidamide based on the procedures set forth in Scheme 3. ¹H-NMR (400 MHz, CD₃OD) δ 8.52 (s, 1H), 7.88-7.85 (m, 2H), 7.78 (d, J=4.0 Hz, 1H), 7.22-7.19 (m, 1H), 7.19-7.14 (m, 2H), 7.11-7.07 (m, 2H), 7.01-6.98 (m, 2H), 5.26 (q, J=7.0 Hz, 1H), 4.84-4.83 (m, 1H), 4.46 (d, J=16.6 Hz, 1H), 4.25 (d, J=16.6 Hz, 1H), 3.67 (d, J=11.7 Hz, 1H), 3.53 (t, J=7.2 Hz, 2H), 2.71 (dd, J=14.9, 9.4 Hz, 1H), 2.10 (dd, J=13.4, 3.8 Hz, 1H), 1.59 (d, J=7.0 Hz, 3H), 1.32 (d, J=3.0 Hz, 1H), 0.99 (t, J=5.8 Hz, 1H). LC/MS (ESI) m/z: 580 (M+H)⁺.

### Scheme 42: Synthesis of (1S,3S,5R)—N—((R)-1-(4-carbamimidoylthiophen-2-yl)ethyl)-2-((4-(4-fluorophenoxy)benzoyl)glycyl)-5-(hydroxymethyl)-2-azabicyclo[3.1.0]hexane-3-carboxamide (Compound 42)

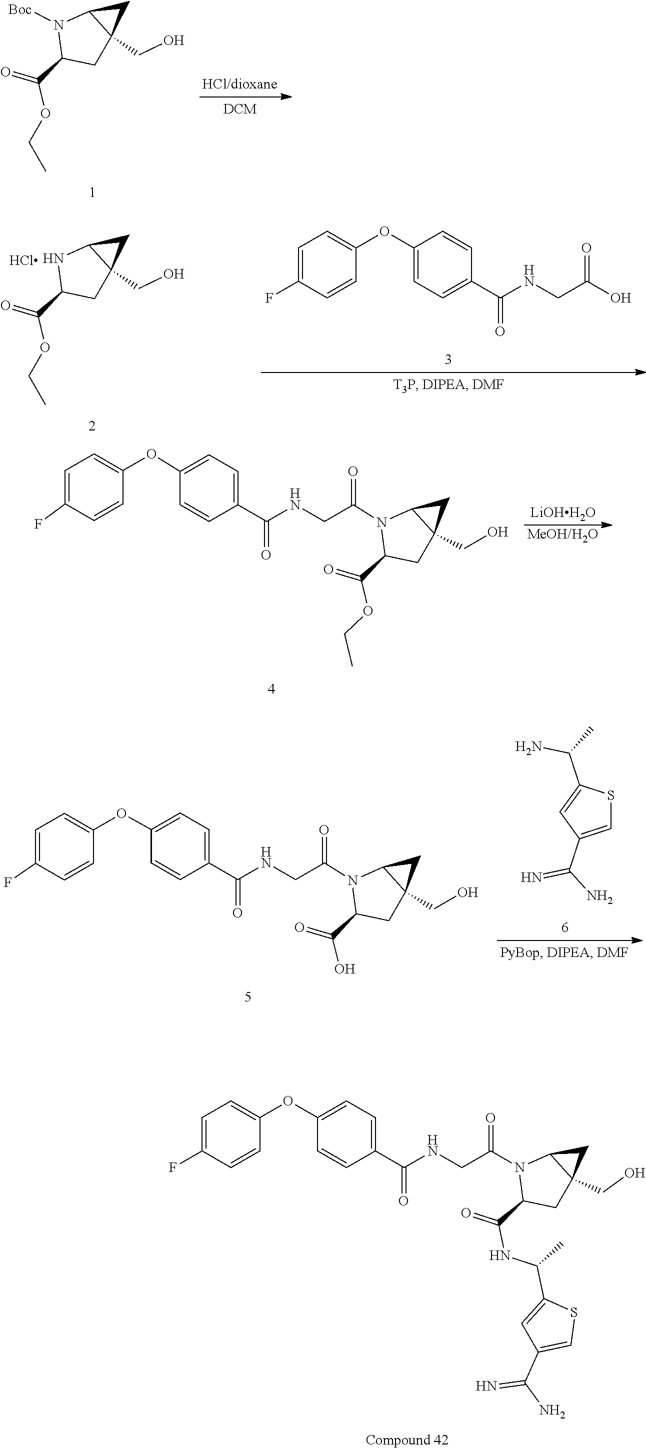

Compound 42 was prepared as a white solid from 2-(tert-butyl)-3-ethyl (1S,3S,5R)-5-(hydroxymethyl)-2-azabicyclo[3.1.0]hexane-2,3-dicarboxylate, (4-(4-fluorophenoxy)benzoyl)glycine, and (R)-5-(1-aminoethyl)thiophene-3-carboximidamide based on the procedure set forth in Scheme 3. ¹H NMR (400 MHz, CD₃OD) δ 8.51 (s, 1H), 8.22 (dd, J=8.0, 1.6 Hz, 1H), 7.86-7.83 (m, 2H), 7.52-7.49 (m, 1H), 7.19-7.14 (m, 2H), 7.11-7.07 (m, 2H), 7.01-6.98 (m, 2H), 5.23 (q, J=7.2 Hz, 1H), 4.84 (s, 1H), 4.41 (d, J=16.5 Hz, 1H), 4.27 (d, J=16.5 Hz, 1H), 3.66 (d, J=11.7 Hz, 1H), 3.54-3.50 (m, 2H), 2.70 (t, J=13.0 Hz, 1H), 2.11 (dd, J=13.4, 3.7 Hz, 1H), 1.58 (d, J=7.0 Hz, 3H), 1.27 (dd, J=5.7, 2.7 Hz, 1H), 0.97 (t, J=5.4 Hz, 1H). LC/MS (ESI) m/z: 580 (M+H)⁺.

### Scheme 43: Synthesis of (2S,4R)—N—((R)-1-(4-carbamimidoylthiophen-2-yl)ethyl)-1-((4-(4-chlorophenoxy)benzoyl)glycyl)-4-(difluoromethoxy)pyrrolidine-2-carboxamide (Compound 43)

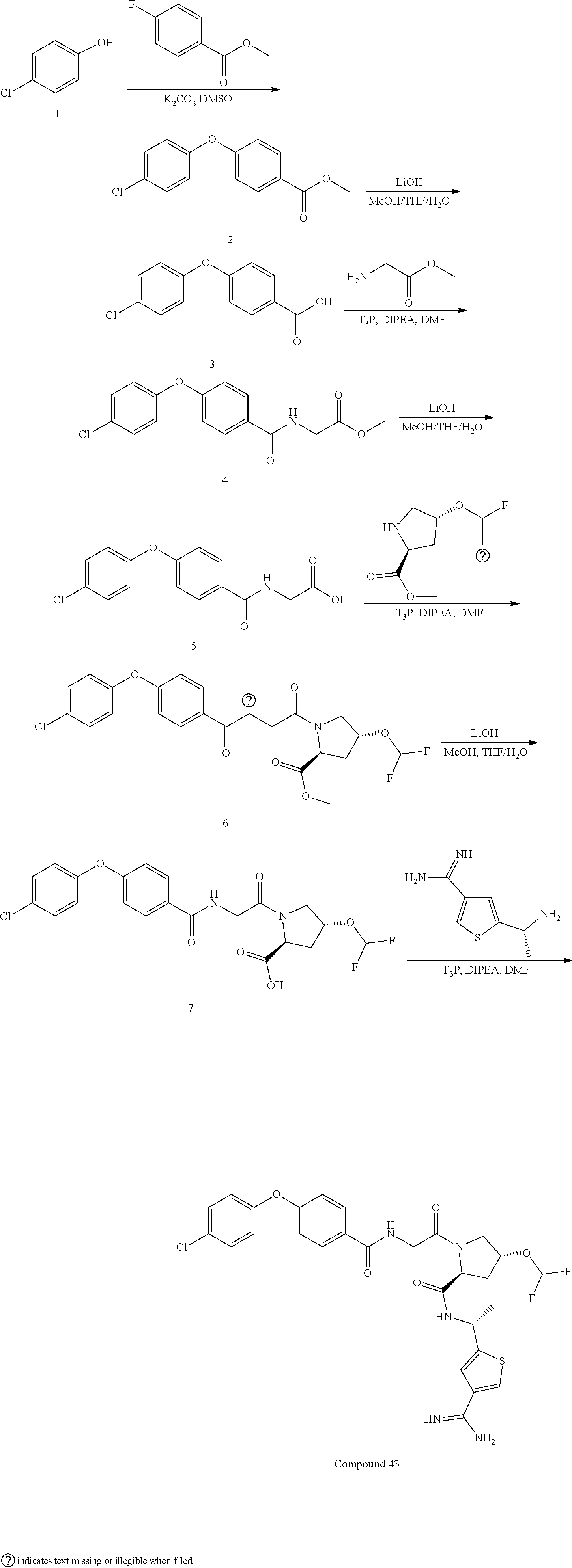

Compound 43 was prepared from 4-chlorophenol, methyl 4-fluorobenzoate, methyl glycinate hydrochloride, methyl (2S,4R)-4-(difluoromethoxy)pyrrolidine-2-carboxylate, and (R)-5-(1-aminoethyl)thiophene-3-carboximidamide based on the procedures set forth in Scheme 24. ¹H NMR (400 MHz, CD₃OD) δ 8.54 (s, 1H), 8.22 (dd, J=9.8, 1.5 Hz, 1H), 7.87 (d, J=8.8 Hz, 2H), 7.54 (d, J=12.5 Hz, 1H), 7.42-7.39 (m, 2H), 7.06-7.02 (m, 4H), 6.71-6.30 (m, 1H), 5.29 (dt, J=20.5, 6.8 Hz, 1H), 5.02 (s, 1H), 4.62 (dt, J=15.5, 7.5 Hz, 2H), 4.20 (dd, J=47.9, 16.7 Hz, 2H), 3.98-3.86 (m, 2H), 2.54-2.40 (m, 1H), 2.29-2.18 (m, 1H), 1.57 (d, J=7.0 Hz, 3H). LCMS (ESI) m/z=620 (M+H)⁺.

### Scheme 44: Synthesis of (1S,3S,5R)—N—((R)-1-(4-carbamimidoylthiophen-2-yl)ethyl)-2-((4-(4-fluorophenoxy)benzoyl)glycyl)-5-(methoxymethyl)-2-azabicyclo[3.1.0]hexane-3-carboxamide (Compound 44)

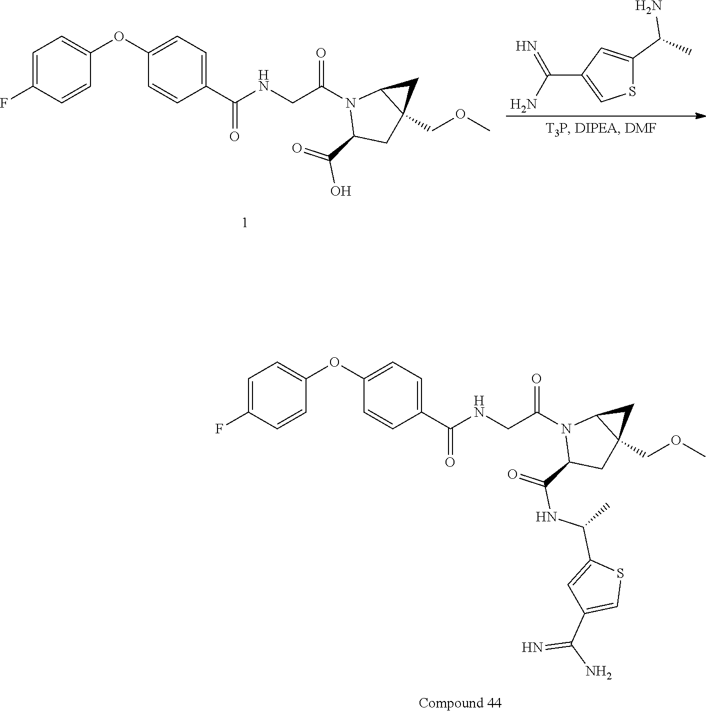 [SMILES: COC[C@@]12C[C@@H]1N(C(=O)CNC(=O)c1ccc(Oc3ccc(F)cc3)cc1)[C@H](C(=O)O)C2 ; C[C@@H](N)c1cc(C(=N)N)cs1 ; COC[C@@]12C[C@@H]1N(C(=O)CNC(=O)c1ccc(Oc3ccc(F)cc3)cc1)[C@H](C(=O)N[C@H](C)c1cc(C(=N)N)cs1)C2]

Compound 44 was prepared as a white solid from (1 S,3S,5R)-2-((4-(4-fluorophenoxy)benzoyl)glycyl)-5-(methoxymethyl)-2-azabicyclo[3.1.0]hexane-3-carboxylic acid and (R)-5-(1-(12-azanyl)ethyl)thiophene-3-carboximidamide based on the procedures set forth in Scheme 32. ¹H NMR (400 MHz, CD₃OD) δ 8.53 (s, 1H), 8.22 (t, J=4.0 Hz, 1H), 7.84 (d, J=8.8 Hz, 2H), 7.50 (s, 1H), 7.16 (t, J=8.6 Hz, 2H), 7.11-7.07 (m, 2H), 6.99 (d, J=8.8 Hz, 2H), 5.22 (q, J=6.8 Hz, 1H), 4.83 (s, 1H), 4.33 (dd, J=56.3, 16.5 Hz, 2H), 3.57 (dd, J=6.1, 2.7 Hz, 1H), 3.50 (d, J=10.4 Hz, 1H), 3.40 (d, J=10.4 Hz, 1H), 3.37 (s, 3H), 2.67 (t, J=12.9 Hz, 1H), 2.11 (dd, J=13.4, 3.7 Hz, 1H), 1.58 (d, J=7.0 Hz, 3H), 1.30 (s, 1H), 0.98 (t, J=5.7 Hz, 1H). LC/MS (ESI) (m/z): 594 (M+H)⁺.

### Scheme 45: Synthesis of (2S,4S)—N—((R)-1-(4-carbamimidoylthiophen-2-yl)ethyl)-1-((4-(4-fluorophenoxy)benzoyl)glycyl)-4-methylpyrrolidine-2-carboxamide (Compound 45)

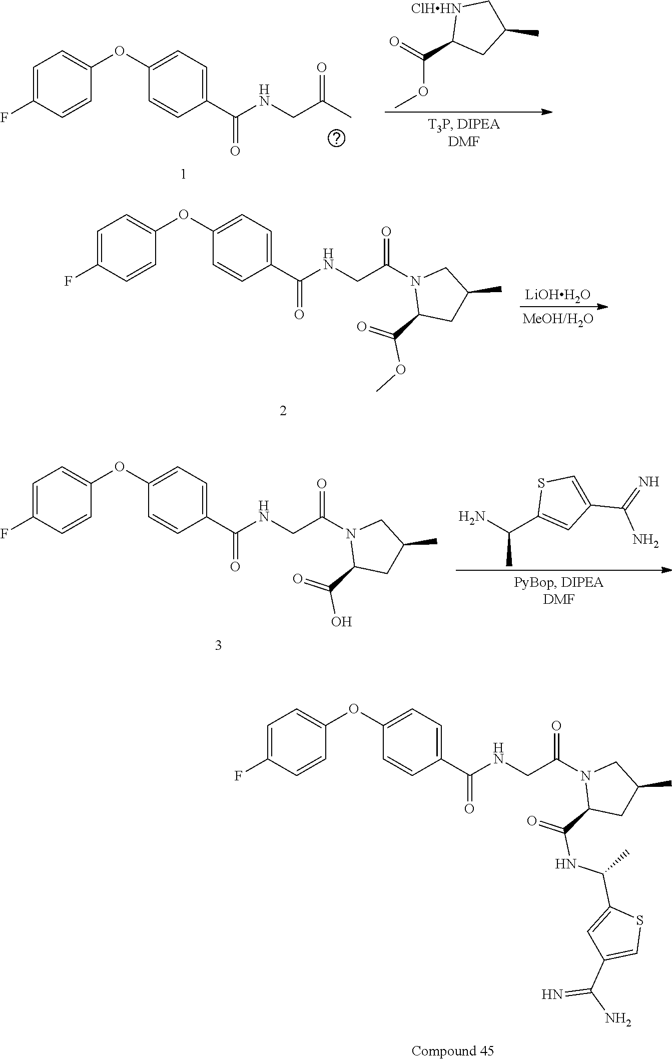

Compound 45 was prepared as a white solid from (4-(4-fluorophenoxy)benzoyl)glycine, methyl (2S,4S)-4-methylpyrrolidine-2-carboxylate hydrochloride, and (R)-5-(1-aminoethyl)thiophene-3-carboximidamide based on the procedures set forth in Scheme 3. ¹H NMR (400 MHz, CD₃OD) δ 8.53 (s, 1H), 8.22 (t, J=3.8 Hz, 1H), 7.85 (d, J=8.8 Hz, 2H), 7.53 (s, 1H), 7.16 (t, J=8.7 Hz, 2H), 7.08 (dd, J=9.1, 4.5 Hz, 2H), 6.99 (d, J=8.8 Hz, 2H), 5.26 (q, J=7.2 Hz, 1H), 4.42-4.36 (m, 1H), 4.29 (d, J=16.6 Hz, 1H), 4.08 (d, J=16.6 Hz, 1H), 3.98-3.92 (m, 1H), 3.18 (t, J=9.9 Hz, 1H), 2.48-2.36 (m, 2H), 1.57 (d, J=7.0 Hz, 3H), 1.38-1.32 (m, 1H), 1.13 (d, J=6.3 Hz, 3H). LC/MS (ESI) m/z: 552 (M+H)⁺.

### Scheme 46: Synthesis of (2S,4S)—N—((R)-1-(4-carbamimidoylthiophen-2-yl)ethyl)-1-((9,9-difluoro-9H-fluorene-3-carbonyl)glycyl)-4-methylpyrrolidine-2-carboxamide (Compound 46)

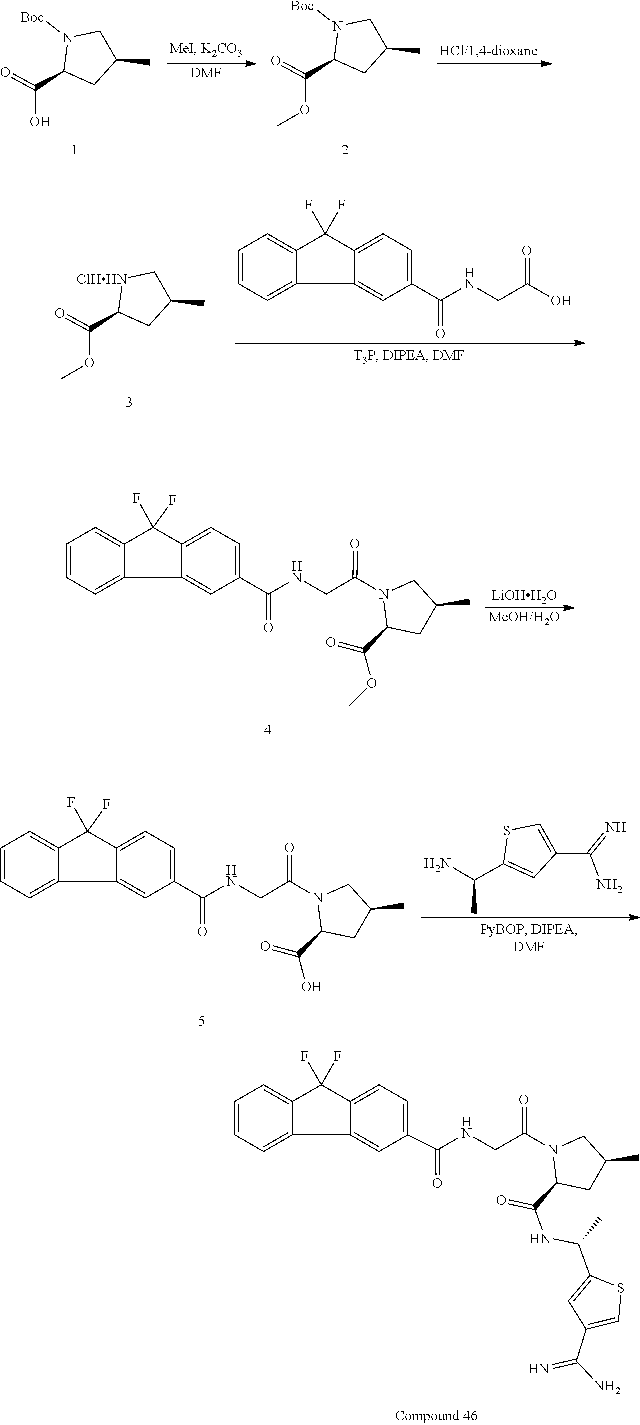

### Step 1: 1-(tert-butyl) 2-methyl (2S,4S)-4-methylpyrrolidine-1,2-dicarboxylate (2)

To a mixture of (2S,4S)-1-(tert-butoxycarbonyl)-4-methylpyrrolidine-2-carboxylic acid (250 mg, 1.09 mmol) and K₂CO₃ (694 mg, 5.03 mmol) in DMF (3 mL) was added MeI (233 mg, 1.64 mmol) at 0° C. under N₂ atmosphere and the mixture was stirred at room temperature overnight. The mixture was diluted with EtOAc, washed with saturated aq·NH₄Cl solution and brine, dried over anhydrous Na₂SO₄, filtered, and concentrated to dryness to give 1-(tert-butyl)-2-methyl (2S,4S)-4-methylpyrrolidine-1,2-dicarboxylate (260 mg, yield 98.1%) as a yellow solid, which was used directly in the next reaction without further purification. LC/MS (ESI) (m/z): 244 (M+H)⁺.

### Step 2: methyl (2S,4S)-4-methylpyrrolidine-2-carboxylate hydrochloride (3)

To a solution of 1-(tert-butyl) 2-methyl (2S,4S)-4-methylpyrrolidine-1,2-dicarboxylate (222 mg, 0.91 mmol) in DCM (5 mL) was added HCl/1,4-dioxane (1.0 mL, 4 mmol) at 0° C. under N₂ atmosphere and the mixture was stirred at room temperature for 1 hour. The mixture was concentrated under reduced pressure to dryness to give methyl (2S,4S)-4-methylpyrrolidine-2-carboxylate hydrochloride (164 mg, yield 100%) as a yellow solid, which was used directly in the next reaction without further purification. LC/MS (ESI) m/z: 144 (M+H)⁺.

### Step 3: methyl (2S,4S)-1-((9,9-difluoro-9H-fluorene-3-carbonyl)glycyl)-4-methylpyrrolidine-2-carboxylate (4)

To a mixture of (9,9-difluoro-9H-fluorene-3-carbonyl)glycine (100 mg, 0.33 mmol) and methyl (2S,4S)-4-methylpyrrolidine-2-carboxylate hydrochloride (90 mg, 0.5 mmol) in DMF (1 mL) was added DIPEA (259 mg, 2.0 mmol) and T₃P (630 mg, 0.99 mmol, 50% wt. in EtOAc) at 0° C. under N₂ atmosphere and the mixture was stirred at room temperature for 1 hour. The mixture was diluted with EtOAc and washed with saturated NaHCO₃ solution. The aqueous layer was extracted with EtOAc twice. The combined organic layers were washed with brine, dried over anhydrous Na₂SO₄, filtered, and concentrated under reduced pressure to dryness. The residue was purified by flash chromatography on silica gel (DCM:MeOH=20:1) to give methyl (2S,4S)-1-((9,9-difluoro-9H-fluorene-3-carbonyl)glycyl)-4-methylpyrrolidine-2-carboxylate (120 mg, yield 84.8%) as a light-yellow solid. LC/MS (ESI) m/z: 429 (M+H)⁺.

### Step 4: (2S,4S)-1-((9,9-difluoro-9H-fluorene-3-carbonyl)glycyl)-4-methylpyrrolidine-2-carboxylic acid (5)

To a solution of methyl (2S,4S)-1-((4-(4-fluorophenoxy)benzoy)glycyl)-4-methylpyrrolidine-2-carboxylate (120 mg, 0.28 mmol) in MeOH (2 mL) and THF (1 mL) was added a solution of LiOH·H₂O (36 mg, 0.84 mmol) in water (1 mL) at 0° C. and the mixture was stirred at room temperature for 2 hours. The mixture was diluted with water and washed with EtOAc twice. The aqueous layer was acidified with 0.5 N aq·HCl to pH˜5 and extracted with EtOAc twice. The combined organic layers were washed with brine, dried over anhydrous Na₂SO₄, filtered, and concentrated to dryness to give (2S,4S)-1-((9,9-difluoro-9H-fluorene-3-carbonyl)glycyl)-4-methylpyrrolidine-2-carboxylic acid (92 mg, yield 79.4%) as a white solid. LC/MS (ESI) (m/z): 415 (M+H)⁺.

### Step 5: (2S,4S)—N—((R)-1-(4-carbamimidoylthiophen-2-yl)ethyl)-1-((9,9-difluoro-9H-fluorene-3-carbonyl)glycyl)-4-methylpyrrolidine-2-carboxamide (Compound 46)

To a mixture of (2S,4S)-1-((9,9-difluoro-9H-fluorene-3-carbonyl)glycyl)-4-methylpyrrolidine-2-carboxylic acid (45 mg, 0.11 mmol) and (R)-5-(1-aminoethyl)thiophene-3-carboximidamide (28 mg, 0.16 mmol) in DMF (0.5 mL) was added DIPEA (36 mg, 0.28 mmol) and PyBOP (68 mg, 0.13 mmol) at 0° C. and the mixture was stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by prep-HPLC to give Compound 46 (4.5 mg, yield 7.2%) as a white solid. ¹HNMR (400 MHz, CD₃OD) δ 8.54 (s, 1H), 8.26-8.16 (m, 2H), 7.92-7.85 (m, 1H), 7.74 (dd, J=16.4, 7.7 Hz, 2H), 7.66 (d, J=7.4 Hz, 1H), 7.57 (dd, J=16.0, 8.5 Hz, 2H), 7.45 (t, J=7.5 Hz, 1H), 5.38-5.25 (m, 1H), 4.47-4.32 (m, 2H), 4.14 (d, J=16.6 Hz, 1H), 4.03-3.94 (m, 1H), 3.21 (t, J=9.8 Hz, 1H), 2.46 (dt, J=17.6, 10.0 Hz, 2H), 1.58 (d, J=7.0 Hz, 3H), 1.39-1.31 (m, 1H), 1.15 (d, J=6.2 Hz, 3H). LC/MS (ESI) m/z: 566 (M+H)⁺.

### Scheme 47: Synthesis of (2S,4R)—N—((R)-1-(4-carbamimidoylthiophen-2-yl)ethyl)-4-(difluoromethoxy)-1-((4-(4-isopropylphenoxy)benzoyl)glycyl)pyrrolidine-2-carboxamide (Compound 47)

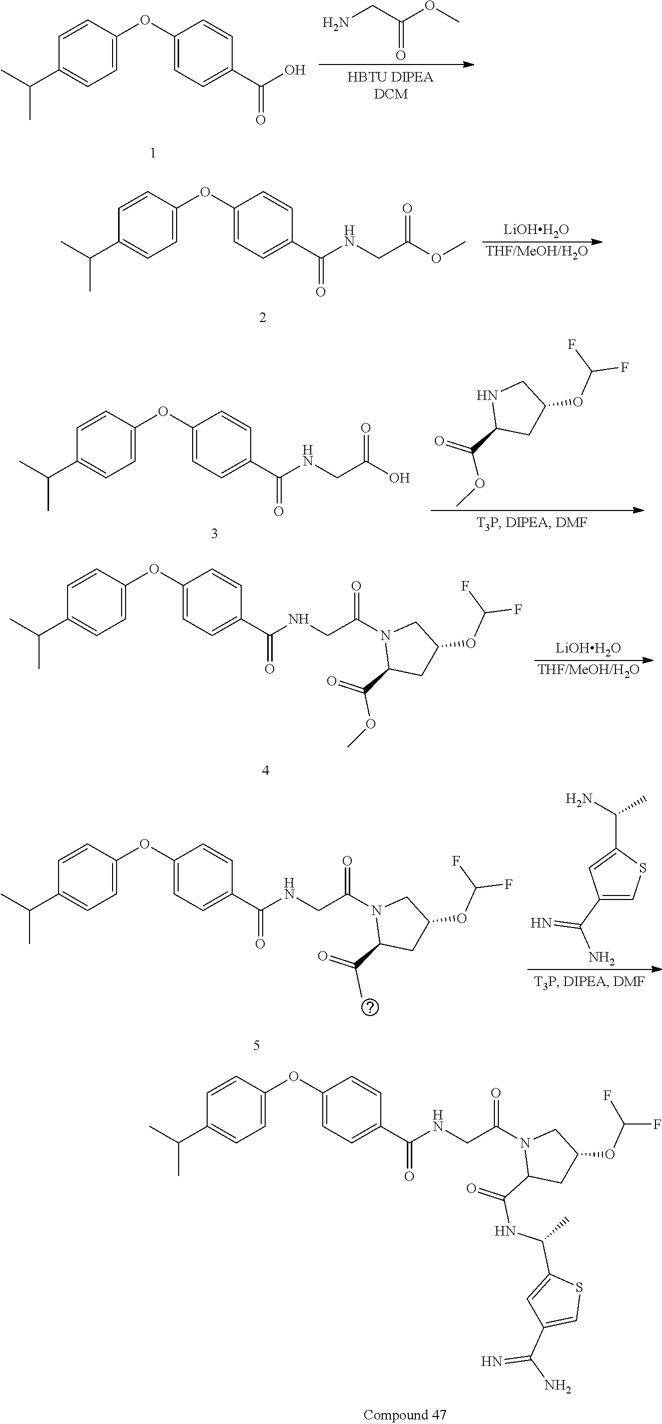

Compound 47 was prepared as a white solid from 4-(4-isopropylphenoxy)benzoic acid, methyl glycinate, methyl (2S,4R)-4-(difluoromethoxy)pyrrolidine-2-carboxylate, and (R)-5-(1-aminoethyl)thiophene-3-carboximidamide based on the procedures set forth in Scheme 24. ¹H NMR (400 MHz, CD₃OD) δ 8.55 (s, 1H), 8.23 (dd, J=7.7, 1.5 Hz, 1H), 7.88-7.81 (m, 2H), 7.54 (d, J=10.7 Hz, 1H), 7.29 (d, J=8.5 Hz, 2H), 6.98 (dd, J=8.6, 1.1 Hz, 4H), 6.50 (td, J=74.5, 15.9 Hz, 1H), 5.26 (q, J=6.7 Hz, 1H), 5.02 (s, 1H), 4.59 (s, 1H), 4.29-4.12 (m, 2H), 3.91 (dt, J=20.3, 8.9 Hz, 2H), 2.94 (dt, J=13.8, 6.8 Hz, 1H), 2.53-2.43 (m, 1H), 2.26-2.19 (m, 1H), 1.62 (dd, J=29.6, 7.0 Hz, 3H), 1.27 (d, J=6.9 Hz, 6H).LC/MS (ESI) m/z: 628 (M+H)⁺.

### Scheme 48. (S)—N-((4-carbamimidoylthiophen-2-yl) methyl)-7-((5-phenylpicolinoyl)glycyl)-1,4-dioxa-7-azaspiro[4.4]nonane-8-carboxamide (Compound 48)

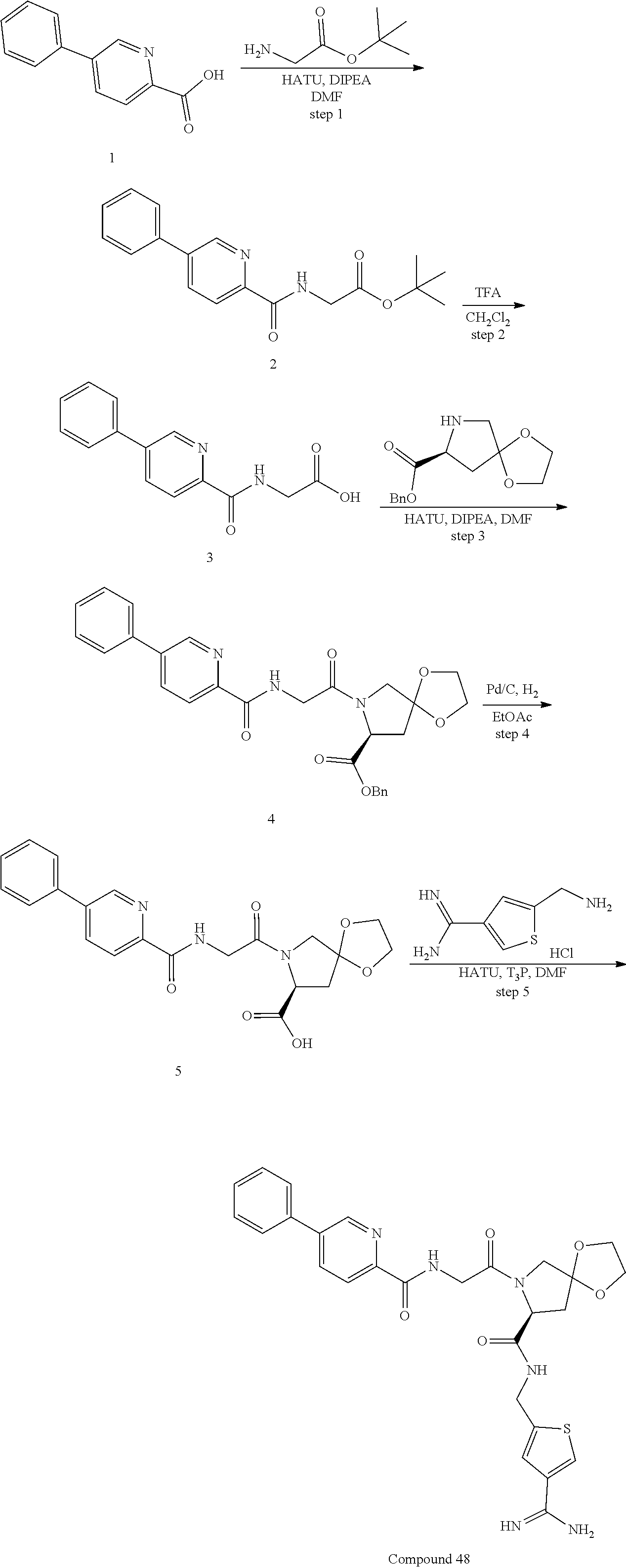

### Step 1: tert-butyl (5-phenylpicolinoyl)glycinate (2)

To a solution of 5-phenylpicolinic acid, 1 (1 g, 5.02 mmol, 1 equiv.) in DMF (5 mL) was added tert-butyl-2-aminoacetate. HCl (1.01 g, 6.02 mmol, 1.2 equiv.), HATU (2.86 g, 7.53 mmol, 1.5 equiv.) and DIPEA (2.64 mL, 15.06 mmol, 3 equiv.). The reaction was stirred at room temperature for 1 hour. Water (10 mL) was added and the solid was collected by filtration and then dried to give compound 2 (1.1 g, 3.52 mmol, yield 70.23%) as a white solid. LC/MS (ESI) m/z: 313 (M+H)+.

### Step 2: (5-phenylpicolinoyl)glycine (3)

TFA (2.0 mL) was added a solution of compound 2 (0.7 g, 2.24 mmol, 1.0 equiv.) in CH₂Cl₂ (5 mL). The reaction was stirred at room temperature for 2 hours and then concentrated to give compound 3 (0.5 g, 1.95 mmol, yield 87.12%) as a white solid which was used in the next reaction without further purification. LC/MS (ESI) m/z: 257 (M+H)+.

### Step 3: benzyl (S)-7-((5-phenylpicolinoyl)glycyl)-1,4-dioxa-7-azaspiro[4.4]nonane-8-carboxylate (4)

To a solution of compound 3 (0.15 g, 0.59 mmol, 1.0 equiv.) in DMF (2 mL) was added benzyl (1S,3S,5S)-5-methyl-2-azabicyclo[3.1.0]hexane-3-carboxylate (0.19 g, 0.72 mmol, 1.2 equiv.), HATU (0.3 g, 0.77 mmol, 1.3 equiv.) and DIPEA (0.31 mL, 1.78 mmol, 3.0 equiv.). The reaction was stirred at room temperature for 30 minutes. Water (5 mL) and EtOAc (5 mL) were added. The two layers were separated, and the aqueous layer was extracted with EtOAc (5 mL×2). The combined organic extracts were washed with brine, dried over Na₂SO₄, and concentrated. Purification by Combi Flash; 12 g column, solvent A=Hexanes, solvent B=EtOAc, 100% A to 25% B gave compound 4 (0.2 g, 0.40 mmol, yield 67.79%) as a brown oil. LC/MS (ESI) m/z: 502 (M+H)+.

### Step 4: (S)-7-((5-phenylpicolinoyl)glycyl)-1,4-dioxa-7-azaspiro[4.4]nonane-8-carboxylic acid (5)

To a solution of compound 4 (0.24 g, 0.48 mmol, 1.0 equiv.) in EtOAc (3 mL) added 10% Pd/C (51 mg, 0.048 mmol, 0.1 equiv.). The flask was evacuated and then backfilled with H₂ in a balloon. The reaction was stirred at room temperature overnight and then filtered through Celite pad. The filtrate was concentrated and dried in vacuo to give compound 5 (0.16 g, 0.39 mmol, yield 81.1%) as a yellow solid. LC/MS (ESI) m/z: 412 (M+H)+.

### Step 5: (S)—N-((4-carbamimidoylthiophen-2-yl)methyl)-7-((5-phenylpicolinoyl)glycyl)-1,4-dioxa-7-azaspiro[4.4]nonane-8-carboxamide (Compound 48)

To a solution of compound 5 (65 mg, 0.16 mmol, 1.0 equiv.) in DMF (5 mL) was added 5-(aminomethyl)thiophene-3-carboximidamide. HCl (0.036 g, 0.19 mmol, 1.1 equiv.), T3P, (50 mass %) in EtOAc (0.14 mL, 0.24 mmol, 1.5 equiv.), and DIPEA (83 μL, 0.48 mmol, 3.0 equiv.). The reaction mixture was stirred at room temperature for 30 minutes and then purified directly by HPLC to give Compound 48 (15 mg, 0.027 mmol, 17.11% yield). LC/MS (ESI) m/z: 549 (M+H)+. ¹H NMR (400 MHz, DMSO-d₆) δ 9.02 (s, 1H), 8.55 (dt, J=19.0, 5.8 Hz, 2H), 8.45 (s, 1H), 8.36-8.29 (m, 1H), 7.83 (dd, J=9.0, 2.5 Hz, 2H), 7.55-7.30 (m, 4H), 7.08 (dd, J=9.1, 2.8 Hz, 2H), 5.17 (s, 1H), 4.71-4.57 (m, 2H), 4.50-4.40 (m, 1H), 4.43-4.33 (m, 3H), 4.31 (dd, J=16.6, 5.6 Hz, 2H), 4.02 (td, J=16.2, 5.6 Hz, 1H), 3.43 (dd, J=6.1, 2.4 Hz, 1H), 2.28 (t, J=12.3 Hz, 1H), 2.09-1.89 (m, 2H)

### Scheme 49: Synthesis of (2S,4R)—N—((R)-1-(4-carbamimidoylthiophen-2-yl)ethyl)-1-((4-(4-cyanophenoxy)benzoyl)glycyl)-4-(difluoromethoxy)pyrrolidine-2-carboxamide (Compound 49)

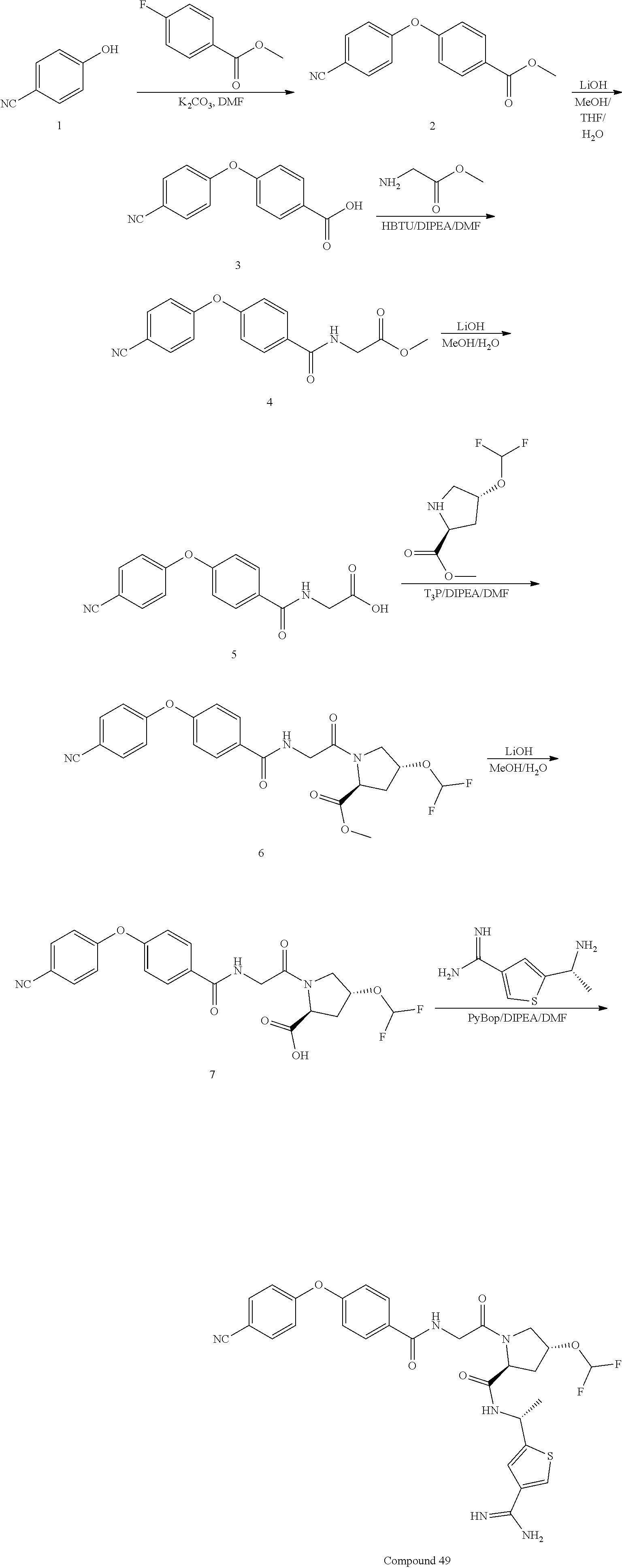

Compound 49 was prepared as a white solid from 4-hydroxybenzonitrile, methyl 4-fluorobenzoate, methyl glycinate hydrochloride, methyl (2S,4R)-4-(difluoromethoxy)pyrrolidine-2-carboxylate hydrochloride, and (R)-5-(1-aminoethyl)thiophene-3-carboximidamide based on the procedures set forth in Scheme 24. ¹H NMR (400 MHz, CD₃OD) δ 8.52 (s, 1H), 8.23 (t, J=4.5 Hz, 1H), 7.95 (d, J=8.8 Hz, 2H), 7.76 (d, J=8.8 Hz, 2H), 7.54 (d, J=11.9 Hz, 1H), 7.17 (dd, J=8.8, 2.8 Hz, 4H), 6.52 (dd, J=82.5, 66.4 Hz, 1H), 5.27 (d, J=7.1 Hz, 1H), 5.03 (s, 1H), 4.58 (t, J=7.8 Hz, 1H), 4.23 (dd, J=47.5, 16.6 Hz, 2H), 3.94 (dd, J=16.4, 11.7 Hz, 2H), 2.50 (d, J=13.4 Hz, 1H), 2.28-2.20 (m, 1H), 1.59 (d, J=7.0 Hz, 3H). LC/MS (ESI) m/z: 611 (M+H)⁺.

### Scheme 50: Synthesis of (2S,4R)—N-((4-carbamimidoylthiophen-2-yl)methyl)-1-((9,9-difluoro-9H-fluorene-3-carbonyl)glycyl)-4-(difluoromethoxy)pyrrolidine-2-carboxamide (Compound 50)

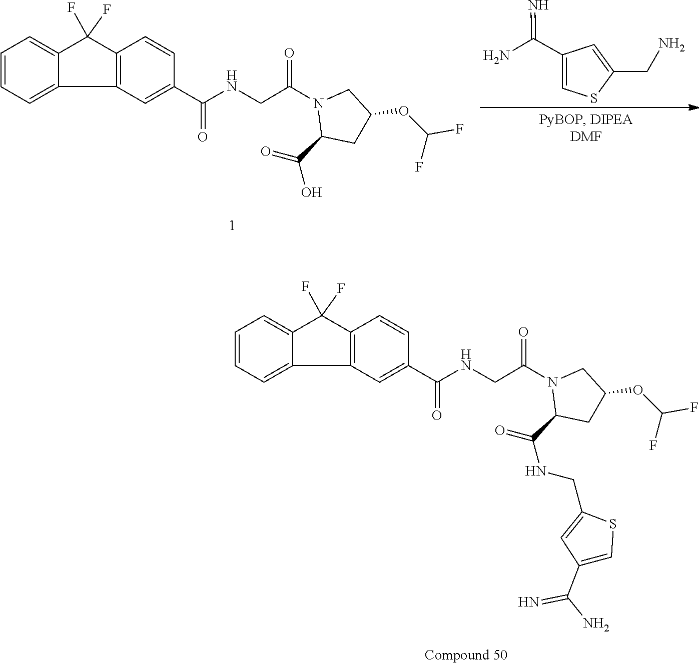 [SMILES: O=C(NCC(=O)N1C[C@H](OC(F)F)C[C@H]1C(=O)O)c1ccc2c(c1)-c1ccccc1C2(F)F ; N=C(N)c1csc(CN)c1 ; N=C(N)c1csc(CNC(=O)[C@@H]2C[C@@H](OC(F)F)CN2C(=O)CNC(=O)c2ccc3c(c2)-c2ccccc2C3(F)F)c1]

Compound 50 was prepared as a white solid from (2S,4R)-1-((9,9-difluoro-9H-fluorene-3-carbonyl)glycyl)-4-(diluoromethoxy)pyrrolidine-2-carboxylic acid and 5-(aminomethyl)thiophene-3-carboximidamide based on the procedures set forth in Scheme 3. ¹H NMR (400 MHz, CD₃OD) δ 8.46 (s, 1H), 8.18 (s, 2H), 7.87 (d, J=7.5 Hz, 1H), 7.77 (d, J=7.7 Hz, 1H), 7.72 (d, J=7.1 Hz, 1H), 7.65 (d, J=7.4 Hz, 1H), 7.58 (t, J=7.2 Hz, 1H), 7.45 (t, J=7.6 Hz, 2H), 6.75-6.24 (m, 1H), 5.03 (s, 1H), 4.59 (s, 3H), 4.26 (s, 2H), 4.01-3.87 (m, 2H), 2.48 (s, 1H), 2.22 (d, J=12.1 Hz, 1H), 2.06 (d, J=19.8 Hz, 1H), 1.30 (s, 3H). LC/MS (ESI) m/z: 604 (M+H)⁺.

### Scheme 51: Synthesis of (2S,4R)—N—((S)-1-(4-carbamimidoylthiophen-2-yl)ethyl)-1-((9,9-difluoro-9H-fluorene-3-carbonyl)glycyl)-4-phenoxypyrrolidine-2-carboxamide (Compound 51)

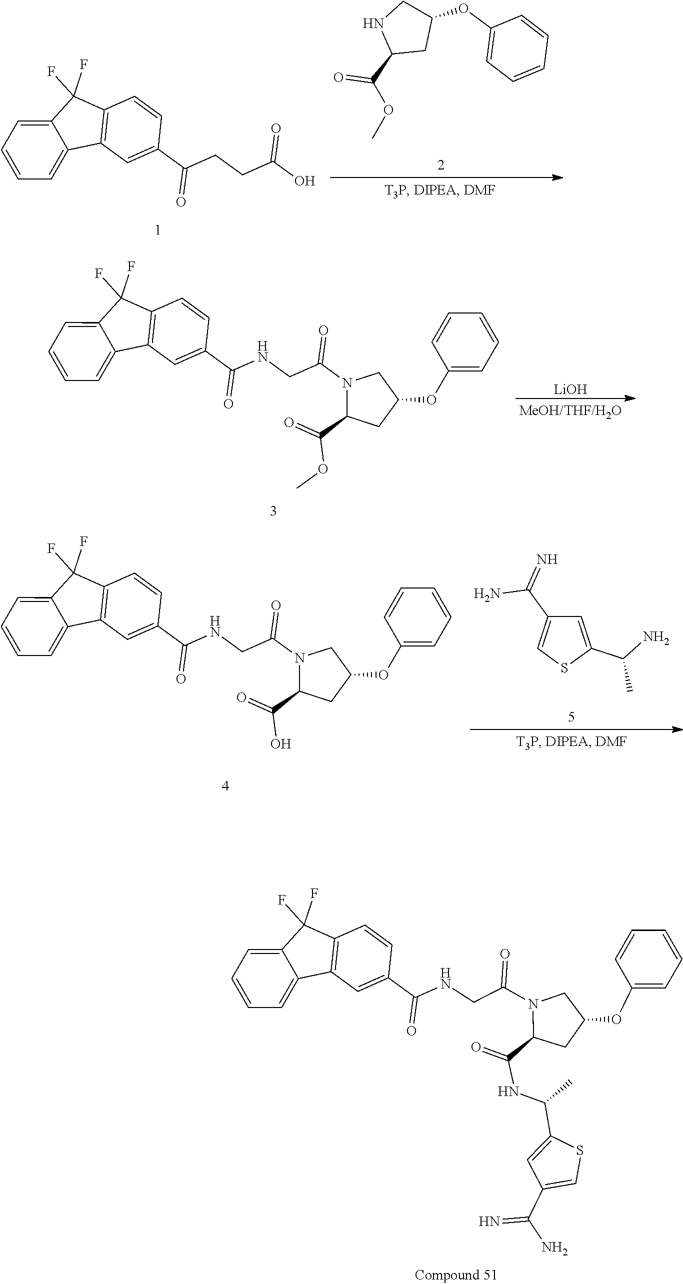

Compound 51 was as a white solid prepared from (9,9-difluoro-9H-fluorene-3-carbonyl)glycine, methyl (2S,4R)-4-phenoxypyrrolidine-2-carboxylate, and (R)-5-(1-aminoethyl)thiophene-3-carboximidamide based on the procedures set forth in Scheme 3. ¹H NMR (400 MHz, CD₃OD) δ 8.52 (s, 1H), 8.28-8.06 (m, 2H), 7.86 (d, J=7.7 Hz, 1H), 7.72 (dd, J=17.9, 7.7 Hz, 2H), 7.64 (d, J=7.7 Hz, 1H), 7.56 (dd, J=14.6, 7.1 Hz, 2H), 7.44 (t, J=7.5 Hz, 1H), 7.33-7.26 (m, 2H), 7.00-6.92 (m, 3H), 5.39-5.25 (m, 1H), 5.14 (d, J=36.4 Hz, 1H), 4.63 (t, J=8.2 Hz, 1H), 4.31 (d, J=16.8 Hz, 1H), 4.18 (d, J=16.7 Hz, 1H), 4.07-3.89 (m, 2H), 2.58 (dd, J=13.0, 7.8 Hz, 1H), 2.30-2.16 (m, 1H), 1.57 (d, J=7.0 Hz, 3H). LC/MS (ESI) (m/z): 644 (M+H)⁺.

### Scheme 52: Synthesis of (2S,4R)—N-((4-carbamimidoylthiophen-2-yl)methyl)-1-((9,9-difluoro-9H-fluorene-3-carbonyl)glycyl)-4-fluoro-4-(fluoromethyl)pyrrolidine-2-carboxamide (Compound 52)

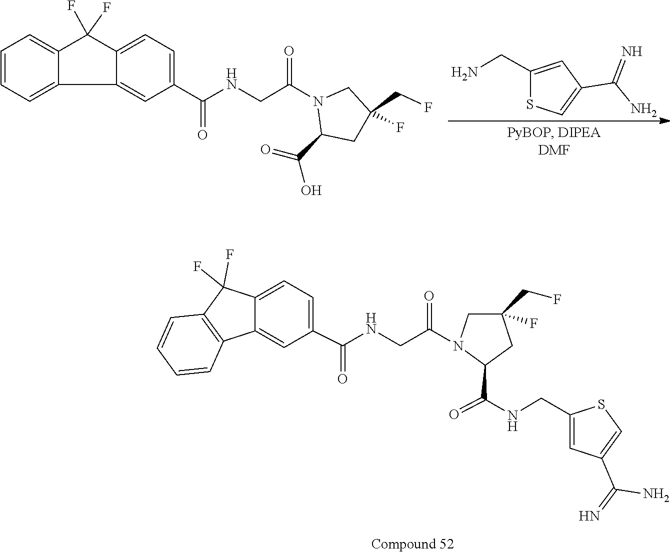

Compound 52 was prepared as a white solid from (2S,4R)-1-((9,9-difluoro-9H-fluorene-3-carbonyl)glycyl)-4-fluoro-4-(fluoromethyl)pyrrolidine-2-carboxylic acid and 5-(aminomethyl)thiophene-3-carboximidamide based on the procedures set forth in Scheme 3. ¹H NMR (400 MHz, CD₃OD) δ 8.28-8.14 (m, 2H), 7.90-7.84 (m, 1H), 7.81-7.75 (m, 1H), 7.72 (d, J=7.6 Hz, 1H), 7.66 (d, J=7.2 Hz, 1H), 7.58 (t, J=7.4 Hz, 1H), 7.54-7.42 (m, 2H), 4.80-4.75 (m, 1H), 4.69-4.66 (m, 1H), 4.65-4.58 (m, 3H), 4.34-4.14 (m, 3H), 4.04-3.92 (m, 1H), 2.73-2.53 (m, 1H), 2.29-2.17 (m, 1H).

### Scheme 53: Synthesis of (1S,3S,5R)—N—((R)-1-(4-carbamimidoylthiophen-2-yl)ethyl)-2-((9,9-difluoro-9H-fluorene-3-carbonyl)glycyl)-5-(hydroxymethyl)-2-azabicyclo [3.1.0]hexane-3-carboxamide (Compound 53)

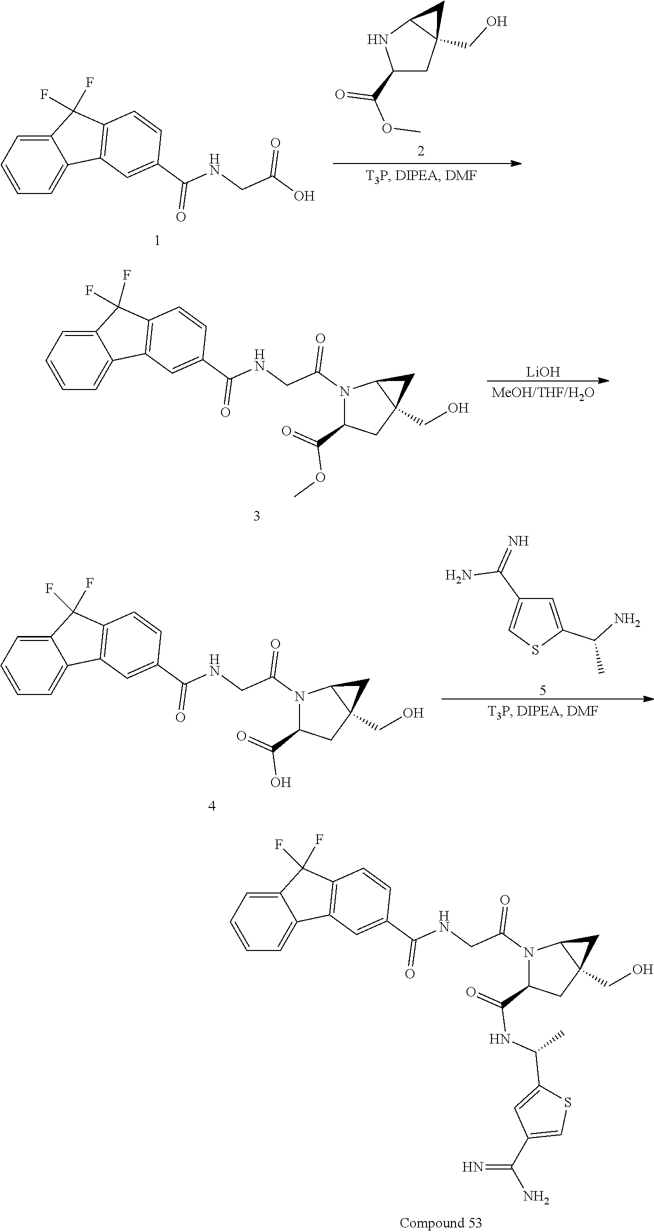

### Step 1: methyl (1S,3S,5R)-2-((9,9-difluoro-9H-fluorene-3-carbonyl)glycyl)-5-(hydroxymethyl)-2-azabicyclo[3.1.0]hexane-3-carboxylate (3)

Compound 53 was prepared as a white solid from (9,9-difluoro-9H-fluorene-3-carbonyl)glycine, methyl (1S,3S,5R)-5-(hydroxymethyl)-2-azabicyclo[3.1.0]hexane-3-carboxylate, and (R)-5-(1-aminoethyl)thiophene-3-carboximidamide based on the procedures set forth in Scheme 3. ¹H NMR (400 MHz, CD₃OD) δ 8.54 (s, 1H), 8.22-8.17 (m, 2H), 7.88 (d, J=7.8 Hz, 1H), 7.74 (dd, J=13.8, 7.8 Hz, 2H), 7.66 (d, J=7.2 Hz, 1H), 7.58 (t, J=7.6 Hz, 1H), 7.46 (dd, J=17.8, 10.2 Hz, 2H), 5.24 (d, J=6.7 Hz, 1H), 4.59-4.45 (m, 2H), 4.32 (d, J=16.5 Hz, 1H), 3.68 (d, J=11.7 Hz, 1H), 3.54 (t, J=9.4 Hz, 2H), 2.72 (t, J=12.0 Hz, 1H), 2.11 (dd, J=13.4, 3.7 Hz, 1H), 1.58 (d, J=7.0 Hz, 3H), 1.05-0.86 (in, 2H). LC/MS (ESI) m/z: 594 (M+H)⁺.

### Scheme 54: (2S,4R)—N—((R)-1-(4-carbamimidoylthiophen-2-yl)ethyl)-1-((4-(4-fluorophenoxy) benzoyl)glycyl)-4-(methylsulfonyl)pyrrolidine-2-carboxamide (Compound 54)

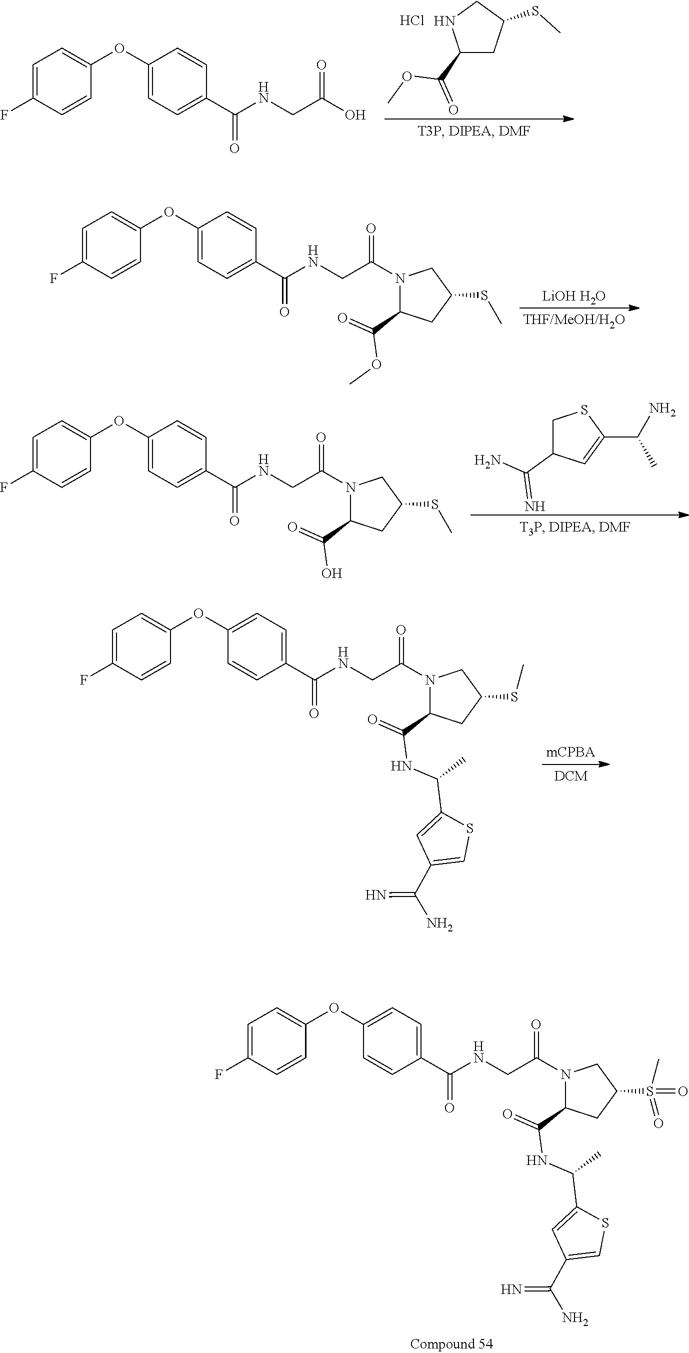

### Step 1: methyl (2S,4R)-1-((4-(4-fluorophenoxy)benzoyl)glycyl)-4-(methylthio)pyrrolidine-2-carboxylate

To a mixture of (4-(4-fluorophenoxy)benzoyl)glycine (96 mg, 0.33 mmol) and methyl (2S,4R)-4-(methylthio)pyrrolidine-2-carboxylate hydrochloride (70 mg, 0.40 mmol) in DMF (1 mL) was added DIPEA (213 mg, 1.65 mmol) and T₃P (315 mg, 0.50 mmol, 50% wt. in EtOAc) at 0° C. under N₂ atmosphere and the mixture was stirred at room temperature for 1 hour. The mixture was diluted with EtOAc and washed with saturated aq·NaHCO₃ solution. The aqueous layer was extracted with EtOAc twice and the combined organic layers were washed with brine, dried over anhydrous Na₂SO₄, filtered, and concentrated under reduced pressure to dryness. The residue was purified by flash chromatography on silica gel (DCM:MeOH=4:1) to give methyl (2S,4R)-1-((4-(4-fluorophenoxy)benzoyl)glycyl)-4-(methylthio)pyrrolidine-2-carboxylate (120 mg, yield 81.1%) as a yellow oil. LC/MS (ESI) m/z: 447 (M+H)⁺.

### Step 2: (2S,4R)-1-((4-(4-fluorophenoxy)benzoyl)glycyl)-4-(methylthio)pyrrolidine-2-carboxylic acid

To a solution of methyl (2S,4R)-1-((4-(4-fluorophenoxy)benzoyl)glycyl)-4-(methylthio)pyrrolidine-2-carboxylate (120 mg, 0.27 mmol) in MeOH (1.5 mL) and THF (0.5 mL) was added a solution of LiOH·H₂O (17 mg, 0.41 mmol) in water (1 mL), and the mixture was stirred at room temperature overnight. The mixture was acidified with 1 N aq·HCl to pH˜4 and extracted with EtOAc twice. The combined organic layers were washed with brine, dried over anhydrous Na₂SO₄, filtered, and concentrated to dryness to give (2S,4R)-1-((4-(4-fluorophenoxy)benzoyl)glycyl)-4-(methylthio)pyrrolidine-2-carboxylic acid (130 mg, crude) as a white solid. LC/MS (ESI) (m/z): 433 (M+H)⁺.

### Step 3: (2S,4R)—N—((R)-1-(4-carbamimidoylthiophen-2-yl)ethyl)-1-((4-(4-fluorophenoxy)benzoyl)glycyl)-4-(methylthio)pyrrolidine-2-carboxamide

To a mixture of (2S,4R)-1-((4-(4-fluorophenoxy)benzoyl)glycyl)-4-(methylthio)pyrrolidine-2-carboxylic acid (130 mg, 0.30 mmol) and (R)-5-(1-aminoethyl)thiophene-3-carboximidamide (102 mg, 0.60 mmol) in DMF (2 mL) was added DIPEA (232 mg, 1.8 mmol) and PyBOP (234 mg, 0.45 mmol) at 0° C. and the mixture was stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography on silica gel (DCM:MeOH=4:1) to give (2S,4R)—N—((R)-1-(4-carbamimidoylthiophen-2-yl)ethyl)-1-((4-(4-fluorophenoxy)benzoyl)glycyl)-4-(methylthio)pyrrolidine-2-carboxamide (80 mg, yield 45.7%) as a yellow oil. LC/MS (ESI) m/z: 584 (M+H)⁺.

### Step 4: (2S,4R)—N—((R)-1-(4-carbamimidoylthiophen-2-yl)ethyl)-1-((4-(4-fluorophenoxy)benzoyl)glycyl)-4-(methylsulfonyl)pyrrolidine-2-carboxamide (Compound 54)

To a solution of (2S,4R)—N—((R)-1-(4-carbamimidoylthiophen-2-yl)ethyl)-1-((4-(4-fluorophenoxy)benzoyl)glycyl)-4-(methylthio)pyrrolidine-2-carboxamide (80 mg, 0.14 mmol) in DCM (1 mL) was added m-CPBA (89 mg, 0.42 mmol) in portions and the mixture was stirred at room temperature for 3 hours. The mixture was washed with saturated aq·NaHCO₃ solution and extracted with DCM twice. The combined organic layers were washed with brine, dried over anhydrous Na₂SO₄, filtered, and concentrated to dryness under reduced pressure. The residue was purified by prep-HPLC to afford Compound 54 (1 mg, yield 1.2%) as a white solid. ¹HNMR (400 MHz, CD₃OD) δ 8.50 (s, 1H), 8.23 (d, J=8.1 Hz, 1H), 7.85 (t, J=8.2 Hz, 2H), 7.52 (d, J=22.4 Hz, 1H), 7.16 (t, J=8.7 Hz, 2H), 7.09 (dd, J=9.1, 4.5 Hz, 2H), 7.00 (d, J=8.7 Hz, 2H), 5.29 (dt, J=19.8, 7.0 Hz, 1H), 4.73 (ddd, J=13.6, 6.9, 3.4 Hz, 1H), 4.26-4.18 (m, 2H), 4.18-4.12 (m, 2H), 4.11-3.90 (m, 1H), 3.06 (d, J=17.6 Hz, 3H), 2.95-2.67 (m, 1H), 2.59-2.37 (m, 1H), 1.63 (dd, J=29.6, 6.9 Hz, 3H). LC/MS (ESI) (m/z): 616 (M+H)⁺.

### Scheme 55: Synthesis of (S)—N-((4-carbamimidoylthiophen-2-yl)methyl)-7-((9,9-difluoro-9H-fluorene-3-carbonyl)glycyl)-1,4-dioxa-7-azaspiro[4.4]nonane-8-carboxamide (Compound 55)

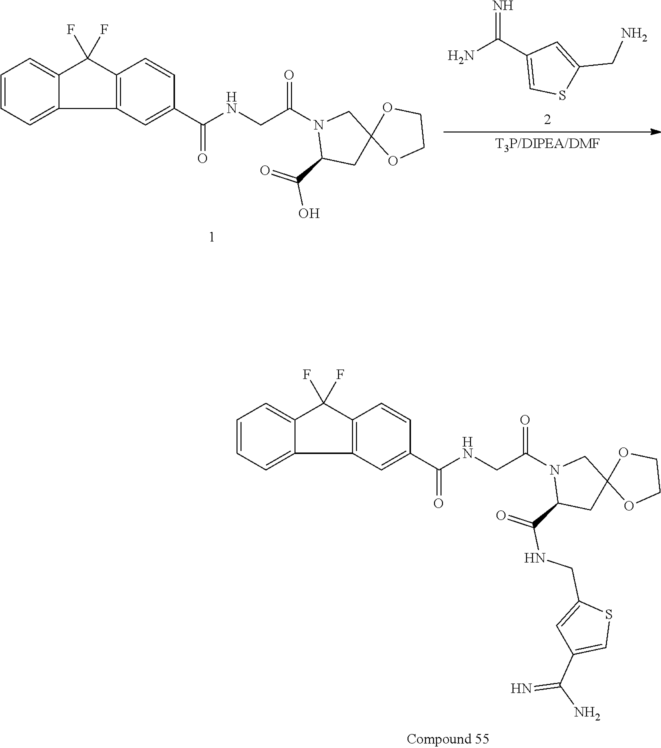 [SMILES: O=C(NCC(=O)N1CC2(C[C@H]1C(=O)O)OCCO2)c1ccc2c(c1)-c1ccccc1C2(F)F ; N=C(N)c1csc(CN)c1 ; N=C(N)c1csc(CNC(=O)[C@@H]2CC3(CN2C(=O)CNC(=O)c2ccc4c(c2)-c2ccccc2C4(F)F)OCCO3)c1]

Compound 55 was prepared as a white solid from (S)-7-((9,9-difluoro-9H-fluorene-3-carbonyl)glycyl)-1,4-dioxa-7-azaspiro[4.4]nonane-8-carboxylic acid and 5-(aminomethyl)thiophene-3-carboximidamide based on the procedures set forth in Scheme 3. ¹H NMR (400 MHz, CD₃OD) δ 8.17 (d, J=5.4 Hz, 1H), 7.98 (dd, J=12.3, 1.4 Hz, 1H), 7.86 (dd, J=7.8, 1.4 Hz, 1H), 7.77 (t, J=8.4 Hz, 1H), 7.71 (d, J=7.8 Hz, 1H), 7.65 (d, J=7.4 Hz, 1H), 7.58 (t, J=7.5 Hz, 1H), 7.46 (d, J=7.4 Hz, 1H), 7.38 (s, 1H), 4.66-4.60 (m, 1H), 4.59 (d, J=4.7 Hz, 2H), 4.31-4.02 (m, 2H), 4.02-3.94 (m, 4H), 3.86-3.73 (m, 2H), 2.59-2.40 (m, 1H), 2.37-2.20 (m, 1H). LC/MS (ESI) m/z: 596 (M+H)⁺.

### Scheme 56: Synthesis of (2S,4S)—N—((R)-1-(4-carbamimidoylthiophen-2-yl)ethyl)-1-((4-(4-fluorophenoxy)benzoyl)glycyl)-4-methylpyrrolidine-2-carboxamide (Compound 56)

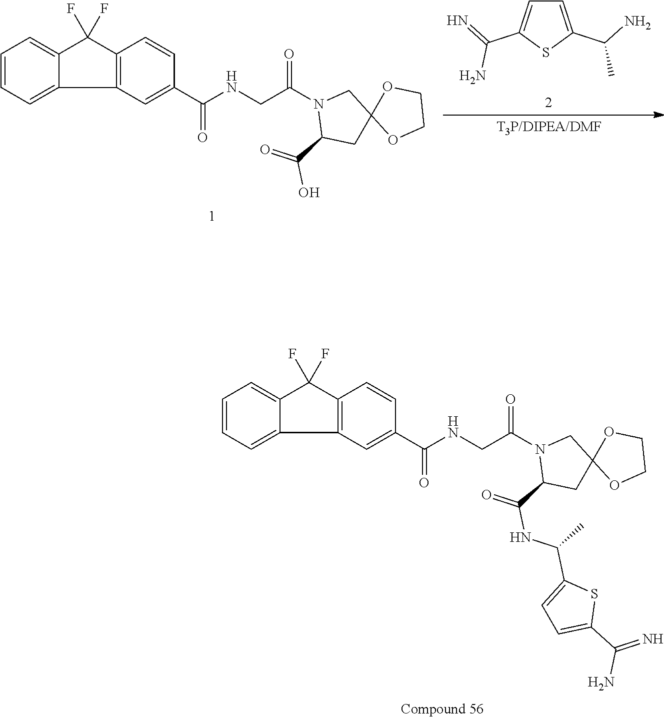 [SMILES: O=C(NCC(=O)N1CC2(C[C@H]1C(=O)O)OCCO2)c1ccc2c(c1)-c1ccccc1C2(F)F ; C[C@@H](N)c1ccc(C(=N)N)s1 ; C[C@@H](NC(=O)[C@@H]1CC2(CN1C(=O)CNC(=O)c1ccc3c(c1)-c1ccccc1C3(F)F)OCCO2)c1ccc(C(=N)N)s1]

Compound 56 was prepared as a white solid from (S)-7-((9,9-difluoro-9H-fluorene-3-carbonyl)glycyl)-1,4-dioxa-7-azaspiro[4.4]nonane-8-carboxylic acid and (R)-5-(1-aminoethyl)thiophene-2-carboximidamide based on the procedures set forth in Scheme 3. ¹H NMR (400 MHz, CD₃OD) δ 8.55 (s, 1H), 8.18 (s, 1H), 7.87 (dt, J=5.3, 2.6 Hz, 1H), 7.76 (dd, J=12.6, 7.7 Hz, 1H), 7.70 (d, J=7.9 Hz, 1H), 7.64 (d, J=7.5 Hz, 1H), 7.57 (t, J=7.6 Hz, 1H), 7.54 (d, J=3.8 Hz, 1H), 7.44 (t, J=7.4 Hz, 1H), 7.12 (dd, J=8.0, 4.1 Hz, 1H), 5.36-5.25 (m, 1H), 4.73-4.53 (m, 1H), 4.29-4.08 (m, 2H), 4.03-3.94 (m, 4H), 3.91-3.74 (m, 2H), 2.43 (dd, J=13.1, 8.8 Hz, 1H), 2.35-2.18 (m, 1H), 1.58 (t, J=16.5 Hz, 3H). LC/MS (ESI) m/z: 610 (M+H)⁺.

### Scheme 57: (2S,4R)—N—((R)-1-(5-carbamimidoylthiophen-2-yl)ethyl)-4-(difluoromethoxy)-1-((4-(4-fluorophenoxy)benzoyl)glycyl)pyrrolidine-2-carboxamide (Compound 57)

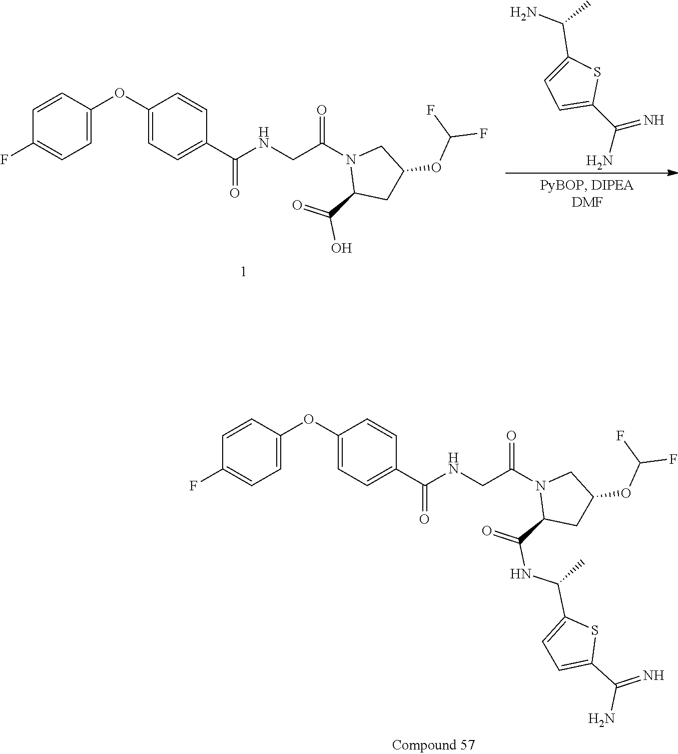 [SMILES: O=C(NCC(=O)N1C[C@H](OC(F)F)C[C@H]1C(=O)O)c1ccc(Oc2ccc(F)cc2)cc1 ; C[C@@H](N)c1ccc(C(=N)N)s1 ; C[C@@H](NC(=O)[C@@H]1C[C@@H](OC(F)F)CN1C(=O)CNC(=O)c1ccc(Oc2ccc(F)cc2)cc1)c1ccc(C(=N)N)s1]

Compound 57 was prepared as a white solid from (2S,4R)-4-(difluoromethoxy)-1-((4-(4-fluorophenoxy)benzoyl)glycyl)pyrrolidine-2-carboxylic acid and (R)-5-(1-aminoethyl)thiophene-2-carboximidamide based on the procedures set forth in Scheme 3. ¹H NMR (400 MHz, CD₃OD) δ 8.53-8.46 (m, 1H), 7.88-7.77 (m, 3H), 7.24 (dd, J=7.6, 7.6 Hz, 1H), 7.18-7.13 (m, 2H), 7.10-7.06 (m, 2H), 7.01-6.97 (m, 2H), 6.76-6.23 (m, 1H), 5.35-5.27 (m, 1H), 5.05-4.99 (m, 1H), 4.57 (t, J=7.8 Hz, 1H), 4.21 (dd, J=64.8, 64.8 Hz, 2H), 3.96-3.85 (m, 2H), 2.55-2.42 (m, 1H), 2.27-2.15 (m, 1H), 1.59 (d, J=7.0 Hz, 3H). LC/MS (ESI) m/z: 604 (M+H)⁺.

### Scheme 58: Synthesis of (2S,4R)—N-((4-carbamimidoylthiophen-2-yl)methyl)-1-((9,9-difluoro-9H-fluorene-3-carbonyl)glycyl)-4-fluoro-4-(methoxymethyl)pyrrolidine-2-carboxamide (Compound 58)

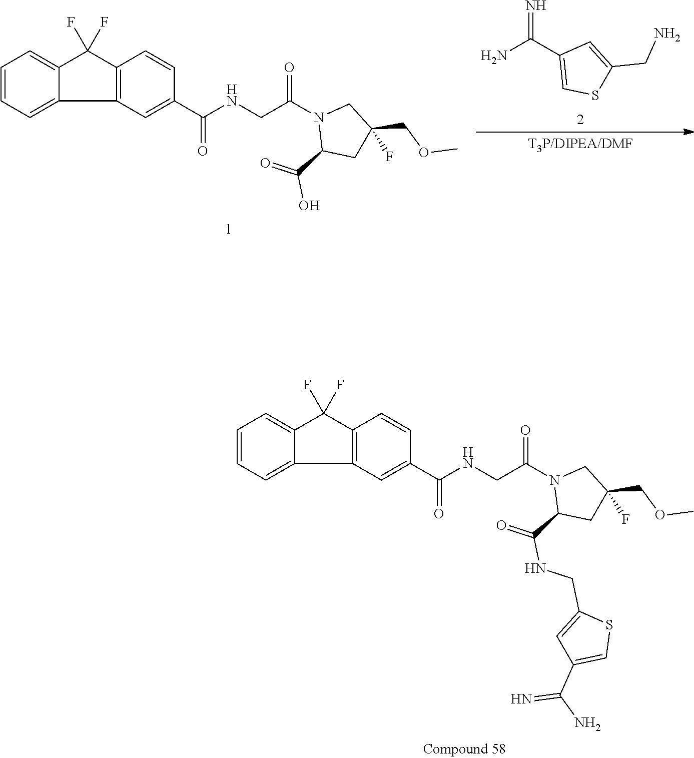 [SMILES: COC[C@@]1(F)C[C@@H](C(=O)O)N(C(=O)CNC(=O)c2ccc3c(c2)-c2ccccc2C3(F)F)C1 ; N=C(N)c1csc(CN)c1 ; COC[C@@]1(F)C[C@@H](C(=O)NCc2cc(C(=N)N)cs2)N(C(=O)CNC(=O)c2ccc3c(c2)-c2ccccc2C3(F)F)C1]

Compound 58 was prepared as a white solid from (2S,4R)-1-((9,9-difluoro-9H-fluorene-3-carbonyl)glycyl)-4-fluoro-4-(methoxymethyl)pyrrolidine-2-carboxylic acid and 5-(aminomethyl)thiophene-3-carboximidamide based on the procedures set forth in Scheme 3. ¹H NMR (400 MHz, CD₃OD) δ 8.55 (s, 1H), 8.16 (s, 1H), 7.94 (dd, J=9.5, 1.4 Hz, 1H), 7.86 (dd, J=7.8, 1.2 Hz, 1H), 7.75 (d, J=7.7 Hz, 1H), 7.70 (d, J=7.8 Hz, 1H), 7.64 (d, J=7.5 Hz, 1H), 7.57 (t, J=7.5 Hz, 1H), 7.45 (d, J=7.7 Hz, 1H), 7.40 (d, J=13.7 Hz, 1H), 4.69-4.60 (m, 1H), 4.58 (d, J=13.0 Hz, 2H), 4.32 (d, J=16.8 Hz, 1H), 4.18 (d, J=16.7 Hz, 1H), 4.06 (dd, J=18.7, 12.4 Hz, 1H), 3.91 (dd, J=33.4, 12.1 Hz, 1H), 3.69 (dd, J=17.7, 5.8 Hz, 2H), 3.42 (d, J=9.4 Hz, 3H), 2.54 (td, J=15.1, 7.9 Hz, 1H), 2.18 (ddd, J=36.4, 14.2, 9.4 Hz, 1H). LC/MS (ESI) m/z: 600 (M+H)⁺.

### Scheme 59: (2S,4R)—N—((R)-1-(4-carbamimidoylthiophen-2-yl)ethyl)-1-((4-(4-fluorophenoxy) benzoyl)glycyl)-4-(o-tolyl)pyrrolidine-2-carboxamide (Compound 59)

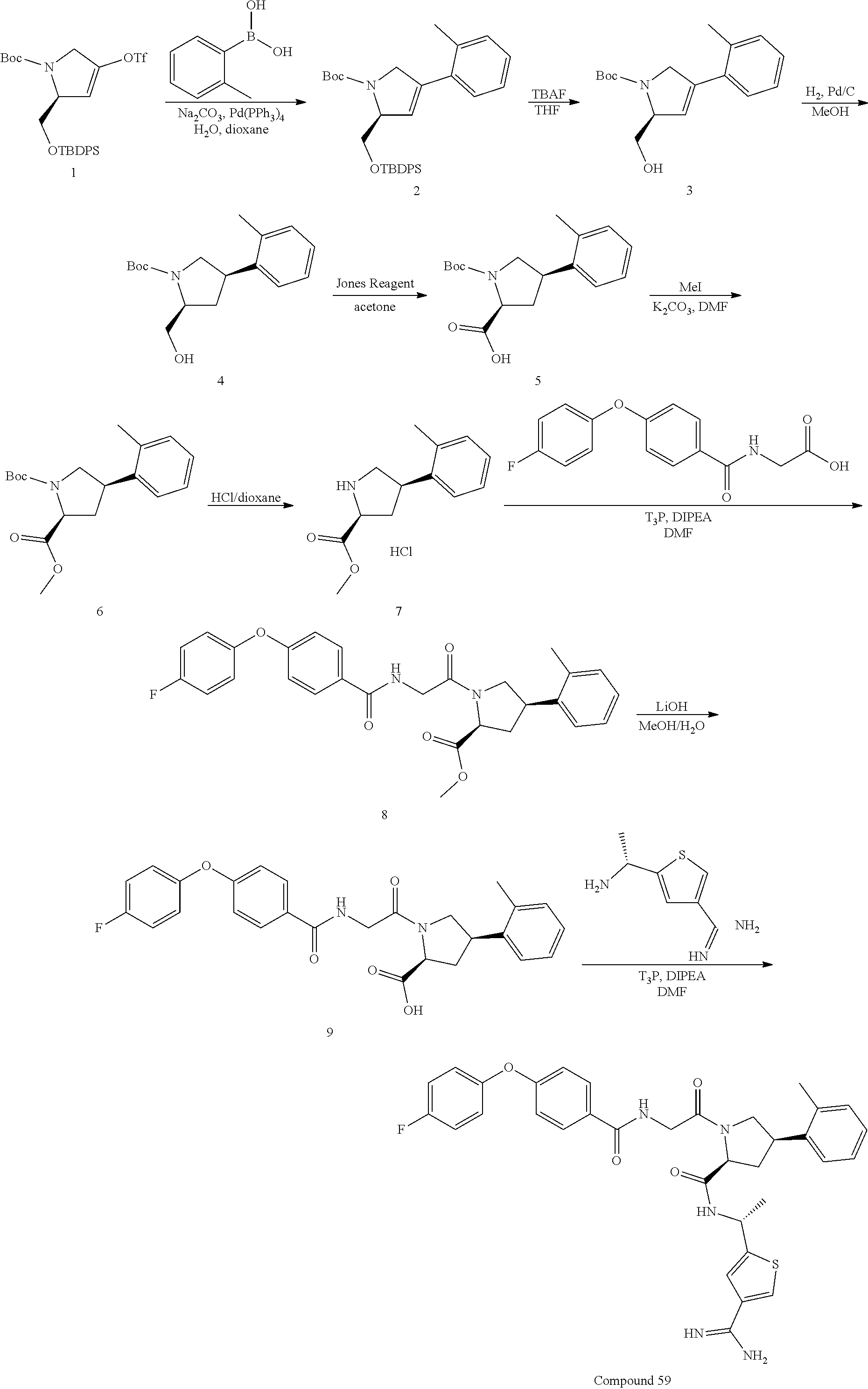

### Step 1: tert-butyl (S)-2-(((tert-butyldiphenylsilyl)oxy)methyl)-4-(o-tolyl)-2,5-dihydro-1H-pyrrole-1-carboxylate (2)

To a solution of tert-butyl (S)-2-(((tert-butyldiphenylsilyl)oxy)methyl)-4-(((trifluoromethyl)sulfonyl)oxy)-2,5-dihydro-1H-pyrrole-1-carboxylate (2.0 g, 3.4 mmol) in 1,4-dioxane (10 mL) and water (10 mL) was added o-tolylboronic acid (927 mg, 6.8 mmol), Pd(PPh₃)₄ (275 mg, 0.24 mmol) and Na₂CO₃ (1.4 g, 13.6 mmol) under N₂ atmosphere at 0° C. The mixture was degassed under N₂ atmosphere for three times and stirred under N₂ atmosphere at 80° C. overnight. The mixture was diluted with water and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=20:1) to give tert-butyl (S)-2-(((tert-butyldiphenylsilyl)oxy)methyl)-4-(o-tolyl)-2,5-dihydro-1H-pyrrole-1-carboxylate (1.7 g, yield 94.4%) as a colorless oil. LCMS (ESI) m/z=528 (M+H)⁺.

### Step 2: tert-butyl (S)-2-(hydroxymethyl)-4-(o-tolyl)-2,5-dihydro-1H-pyrrole-1-carboxylate (3)

To a solution of tert-butyl (S)-2-(((tert-butyldiphenylsilyl)oxy)methyl)-4-(o-tolyl)-2,5-dihydro-1H-pyrrole-1-carboxylate (1.7 g, 3.2 mmol) in THF (17 mL) was added TBAF (6.5 mL, 6.5 mmol) at 0° C. and the mixture was stirred under N₂ atmosphere at room temperature for 2 hours. The mixture was diluted EtOAc, washed with saturated aq·NH₄Cl solution and brine, dried over anhydrous Na₂SO₄, filtered, and concentrated under reduced pressure to dryness. The residue was purified by flash chromatography on silica gel (PE:EtOAc=3:1) to give tert-butyl (S)-2-(hydroxymethyl)-4-(o-tolyl)-2,5-dihydro-1H-pyrrole-1-carboxylate (350 mg, yield 37.0%) as a colorless oil. LCMS (ESI) m/z=290 (M+H)⁺.

### Step 3: tert-butyl (2S,4R)-2-(hydroxymethyl)-4-(o-tolyl)pyrrolidine-1-carboxylate (4)

To a solution of tert-butyl (S)-2-(hydroxymethyl)-4-(o-tolyl)-2,5-dihydro-1H-pyrrole-1-carboxylate (320 mg, 1.1 mmol) in MeOH (5 mL) was added Pd/C (50 mg, 10% wt), the mixture was degassed under N₂ atmosphere for three times and stirred under a H₂ balloon at room temperature overnight. The mixture was filtered, and the filtrate was concentrated to dryness under reduced pressure to give tert-butyl (2S,4S)-2-(hydroxymethyl)-4-(o-tolyl)pyrrolidine-1-carboxylate (320 mg, yield 99.8%) as a colorless oil, which was used directly in the next reaction. LC/MS (ESI) (m/z): 292 (M+H)⁺.

### Step 4: (2S,4R)-1-(tert-butoxycarbonyl)-4-(o-tolyl)pyrrolidine-2-carboxylic acid (5)

To a solution of tert-butyl (2S,4S)-2-(hydroxymethyl)-4-(o-tolyl)pyrrolidine-1-carboxylate (130 mg, 0.45 mmol) in acetone (1.5 mL) was added newly prepared Jones reagent (0.4 mL, 1.1 mmol) under N₂ atmosphere at 0° C. and the mixture was stirred at 25° C. for 1 hour. The mixture was quenched with isopropanol and diluted with water. The mixture was extracted with EtOAc twice and the combined organic layers were washed with brine, dried over anhydrous Na₂SO₄, filtered, and concentrated under reduced pressure to dryness. The residue was purified by flash chromatography on silica gel (DCM:MeOH=12:1) to give (2S,4S)-1-(tert-butoxycarbonyl)-4-(o-tolyl)pyrrolidine-2-carboxylic acid (120 mg, yield 88.1%) as a yellow oil. LCMS (ESI) m/z=306 (M+H)⁺.

### Step 5: 1-(tert-butyl) 2-methyl (2S,4R)-4-(o-tolyl)pyrrolidine-1,2-dicarboxylate (6)

To a mixture of (2S,4S)-1-(tert-butoxycarbonyl)-4-(o-tolyl)pyrrolidine-2-carboxylic acid (120 mg, 0.39 mmol) and K₂CO₃ (216 mg, 1.6 mmol) in DMF (2.0 mL) was added iodomethane (129 mg, 0.76 mmol) and the mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography on silica gel (PE:EtOAc=3:1) to give 1-(tert-butyl) 2-methyl (2S,4S)-4-(o-tolyl)pyrrolidine-1,2-dicarboxylate (97 mg, yield 77.3%) as a yellow oil. LCMS (ESI) m/z=320 (M+H)⁺.

### Step 6: methyl (2S,4R)-4-(o-tolyl)pyrrolidine-2-carboxylate hydrochloride (7)

A mixture of 1-(tert-butyl) 2-methyl (2S,4S)-4-(o-tolyl)pyrrolidine-1,2-dicarboxylate (97 mg, 0.30 mmol) in HCl/1,4-dioxane (1.5 mL) was stirred at room temperature for 30 minutes. The reaction mixture was concentrated to dryness under reduced pressure, co-evaporeated with DCM and dried under vacuum to give methyl (2S,4S)-4-(o-tolyl)pyrrolidine-2-carboxylate hydrochloride (92 mg, yield 100%) as a yellow solid, which was used directly in the next reaction without further purification. LC/MS (ESI) m/z: 220 (M+H)⁺.

### Step 7: methyl (2S,4R)-1-((4-(4-fluorophenoxy)benzoyl)glycyl)-4-(o-tolyl)pyrrolidine-2-carboxylate (8)

To a mixture of (4-(4-fluorophenoxy)benzoyl)glycine (40 mg, 0.14 mmol) and methyl (2S,4S)-4-(o-tolyl)pyrrolidine-2-carboxylate hydrochloride (36 mg, 0.14 mmol) in DMF (1.0 mL) was added DIPEA (71 mg, 0.55 mmol) and T₃P (57 mg, 0.18 mmol) at 0° C. and the mixture was stirred under N₂ atmosphere at room temperature for 1 hour. The mixture was quenched with saturated aq·NaHCO₃ solution and extracted with EtOAc twice. The combined organic layers were washed with brine, dried over with anhydrous Na₂SO₄, filtered, and concentrated to dryness under reduced pressure. The residue was purified by flash chromatography on silica gel (PE:EtOAc=1:1) to give methyl (2S,4S)-1-((4-(4-fluorophenoxy)benzoyl)glycyl)-4-(o-tolyl)pyrrolidine-2-carboxylate (60 mg, yield 89.3%) as a yellow oil.

### Step 8: (2S,4R)-1-((4-(4-fluorophenoxy)benzoyl)glycyl)-4-(o-tolyl)pyrrolidine-2-carboxylic acid (9)

To a solution of methyl (2S,4R)-1-((4-(4-fluorophenoxy)benzoyl)glycyl)-4-(o-tolyl)pyrrolidine-2-carboxylate (35 mg, 0.071 mmol) in MeOH (0.3 mL), THF (0.3 mL) and water (0.3 mL) was added LiOH (17 mg, 0.43 mmol) at 0° C. and the mixture was stirred at room temperature for 1 hour. The mixture was acidified with 1 N aq·HCl to pH-4 and extracted with EtOAc twice. The combined organic layers were washed with brine, dried over anhydrous Na₂SO₄, filtered, and concentrated under reduced pressure to dryness to give (2S,4R)-1-((4-(4-fluorophenoxy)benzoyl)glycyl)-4-(o-tolyl)pyrrolidine-2-carboxylic acid (25 mg, yield 73.9%) as a light-yellow solid, which was used directly in the next step. LC/MS (ESI) (m/z): 477 (M+H)⁺.

### Step 9: (2S,4R)—N—((R)-1-(4-carbamimidoylthiophen-2-yl)ethyl)-1-((4-(4-fluorophenoxy)benzoyl)glycyl)-4-(o-tolyl)pyrrolidine-2-carboxamide (Compound 59)

To a mixture of (2S,4R)-1-((4-(4-fluorophenoxy)benzoyl)glycyl)-4-(o-tolyl)pyrrolidine-2-carboxylic acid (25 mg, 0.052 mmol) and 5-(aminomethyl)thiophene-3-carboximidamide (14 mg, 0.079 mmol) in DMF (1.0 mL) was added DIPEA (41 mg, 0.31 mmol) and T₃P (22 mg, 0.068 mmol) at 0° C. and the mixture was stirred under N₂ atmosphere at room temperature for 1 hour. The mixture was quenched with saturated aq·NaHCO₃ solution and extracted with EtOAc twice. The combined organic layers were washed with brine, dried over anhydrous Na₂SO₄, filtered, and concentrated under reduced pressure to dryness. The residue was purified by flash chromatography on silica gel (DCM:MeOH=12:1) to give (2S,4R)—N-((4-carbamimidoylthiophen-2-yl)methyl)-1-((4-(4-fluorophenoxy)benzoyl)glycyl)-4-(o-tolyl)pyrrolidine-2-carboxamide (3.5 mg, yield 10.7%) as a white solid. ¹H NMR (400 MHz, CD₃OD) δ 8.55 (s, 1H), 8.23 (t, J=3.8 Hz, 1H), 7.88-7.84 (m, 2H), 7.56 (s, 1H), 7.34 (d, J=7.4 Hz, 1H), 7.21-7.14 (m, 5H), 7.10-7.06 (m, 2H), 6.99 (d, J=8.8 Hz, 2H), 5.30 (q, J=7.1 Hz, 1H), 4.54 (dd, J=9.9, 7.4 Hz, 1H), 4.35 (d, J=16.6 Hz, 1H), 4.21-4.16 (m, 1H), 4.11 (d, J=16.6 Hz, 1H), 3.79 (td, J=11.4, 6.2 Hz, 1H), 3.67-3.62 (m, 1H), 2.64 (dt, J=13.3, 6.7 Hz, 1H), 2.42 (s, 3H), 2.12-2.04 (m, 1H), 1.59 (d, J=7.0 Hz, 3H). LCMS (ESI) m/z=628 (M+H)⁺.

### Scheme 60: Synthesis of (S)—N-((4-carbamimidoylthiophen-2-yl)methyl)-7-((9,9-dimethyl-9H-fluorene-3-carbonyl)glycyl)-1,4-dioxa-7-azaspiro[4.4]nonane-8-carboxamide (Compound 60)

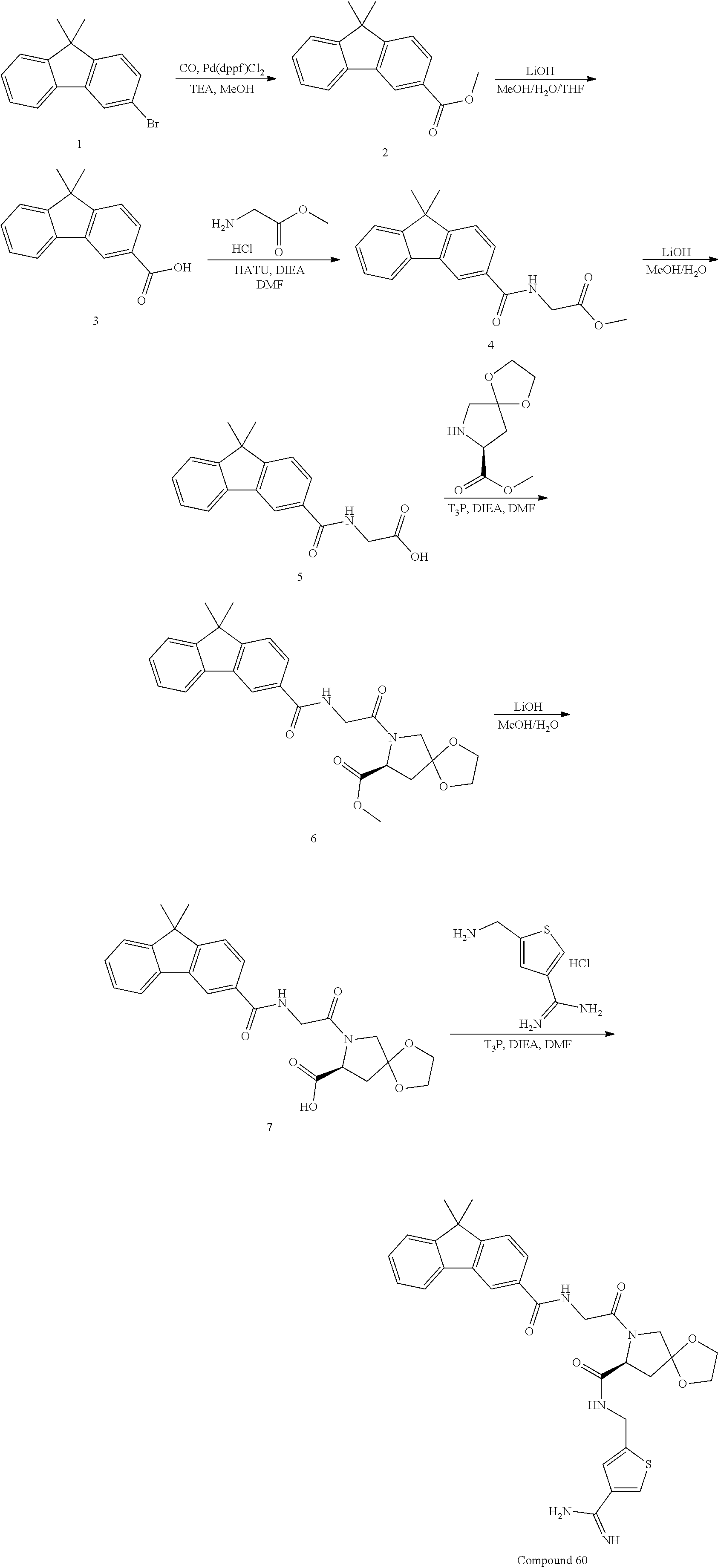

### Step 1: methyl 9,9-dimethyl-9H-fluorene-3-carboxylate (2)

To a solution of 3-bromo-9,9-dimethyl-9H-fluorene (500 mg, 1.8 mmol) in MeOH (5.0 mL) was added TEA (273 mg, 2.7 mmol) and Pd(dppf)Cl₂ (95 mg, 0.13 mmol). The mixture was degassed under N₂ atmosphere at 0° C. and stirred under CO balloon at 70° C. overnight. The mixture was diluted with water and extracted with EtOAc twice. The combined organic layers were washed with brine, dried over anhydrous Na₂SO₄, filtered, and concentrated under reduced pressure to dryness. The residue was purified by column chromatography on silica gel (PE:EtOAc=3:1) to give methyl 9,9-dimethyl-9H-fluorene-3-carboxylate (370 mg, yield 80.3%) as a colorless oil. LCMS (ESI) m/z=253 (M+H)⁺.

### Step 2: 9,9-dimethyl-9H-fluorene-3-carboxylic acid (3)

To a solution of methyl 9,9-dimethyl-9H-fluorene-3-carboxylate (370 mg, 1.5 mmol) in MeOH (3 mL) and water (0.6 mL) was added lithium hydroxide (350 mg, 8.8 mmol) at 0° C. and the mixture was stirred at room temperature for 2 hours. The mixture was acidified with 1 N aq·HCl to pH-3, extracted with ethyl acetate twice. The combined organic layers were washed with brine, dried over anhydrous Na₂SO₄, filtered, and concentrated under reduced pressure to dryness to give 9,9-dimethyl-9H-fluorene-3-carboxylic acid (340 mg, yield 97.2%) as a colorless oil, which was used directly in the next reaction. LC/MS (ESI) (m/z): 239 (M+H)⁺.

### Step 3: methyl (9,9-dimethyl-9H-fluorene-3-carbonyl)glycinate (4)

To a mixture of 9,9-dimethyl-9H-fluorene-3-carboxylic acid (340 mg, 1.4 mmol) and methyl glycinate hydrochloride (180 mg, 1.4 mmol) in DMF (4.0 mL) was added DIEA (1.1 g, 8.5 mmol) and HBTU (808 mg, 2.1 mmol) at 0° C. and the mixture was stirred under N₂ atmosphere at room temperature for 1 hour. The mixture was diluted with water and extracted with EtOAc twice. The combined organic layers were washed with brine, dried over with anhydrous Na₂SO₄, filtered, and concentrated to dryness under reduced pressure. The residue was purified by flash chromatography on silica gel (PE:EtOAc=1:1) to give methyl (9,9-dimethyl-9H-fluorene-3-carbonyl)glycinate (360 mg, yield 81.7%) as a white solid. LCMS (ESI) m/z=310 (M+H)⁺.

### Step 4: (9,9-dimethyl-9H-fluorene-3-carbonyl)glycine (5)

To a solution of methyl (9,9-dimethyl-9H-fluorene-3-carbonyl)glycinate (360 mg, 1.2 mmol) in MeOH (2 mL) and water (2 mL) was added lithium hydroxide (278 mg, 7.0 mmol) at 0° C. and the mixture was stirred at room temperature for 2 hours. The mixture was acidified with 1 N aq·HCl to pH-3, extracted with ethyl acetate twice. The combined organic layers were washed with brine, dried over with anhydrous Na₂SO₄, filtered, and concentrated under reduced pressure to dryness to give (9,9-dimethyl-9H-fluorene-3-carbonyl)glycine (320 mg, yield 93.5%) as a colorless oil, which was used directly in the next reaction. LC/MS (ESI) (m/z): 296 (M+H)⁺.

### Step 5: methyl (S)-7-((9,9-dimethyl-9H-fluorene-3-carbonyl)glycyl)-1,4-dioxa-7-azaspiro[4.4]nonane-8-carboxylate (6)

To a mixture of (9,9-dimethyl-9H-fluorene-3-carbonyl)glycine (100 mg, 0.34 mmol) and methyl (S)-1,4-dioxa-7-azaspiro[4.4]nonane-8-carboxylate (63 mg, 0.34 mmol) in DMF (1.5 mL) was added DIEA (260 mg, 2.0 mmol) and T₃P (118 mg, 0.37 mmol) at 0° C. and the mixture was stirred under N₂ atmosphere at room temperature for 1 hour. The mixture was quenched with saturated aq·NaHCO₃ solution and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=1:1) to give methyl (S)-7-((9,9-dimethyl-9H-fluorene-3-carbonyl)glycyl)-1,4-dioxa-7-azaspiro[4.4]nonane-8-carboxylate (122 mg, yield 77.3%) as a white solid. LCMS (ESI) m/z=465 (M+H)⁺.

### Step 6: (S)-7-((9,9-dimethyl-9H-fluorene-3-carbonyl)glycyl)-1,4-dioxa-7-azaspiro[4.4]nonane-6-carboxylic acid (7)

To a solution of methyl (S)-7-((9,9-dimethyl-9H-fluorene-3-carbonyl)glycyl)-1,4-dioxa-7-azaspiro[4.4]nonane-8-carboxylate (100 mg, 0.34 mmol) in MeOH (1.0 mL) and water (0.5 mL) was added lithium hydroxide (63 mg, 1.6 mmol) at 0° C. and the mixture was stirred at room temperature for 2 hours. The mixture was acidified with 1 N aq·HCl to pH˜3, extracted with ethyl acetate twice. The combined organic layers were washed with brine, dried over anhydrous Na₂SO₄, filtered, and concentrated under reduced pressure to dryness to give (S)-7-((9,9-dimethyl-9H-fluorene-3-carbonyl)glycyl)-1,4-dioxa-7-azaspiro[4.4]nonane-8-carboxylic acid (102 mg, yield 88.5%) as a colorless oil, which was used directly in the next reaction. LC/MS (ESI) (m/z): 451 (M+H)⁺.

### Step 7: (S)—N-((4-carbamimidoylthiophen-2-yl)methyl)-7-((9,9-dimethyl-9H-fluorene-3-carbonyl)glycyl)-1,4-dioxa-7-azaspiro[4.4]nonane-8-carboxamide hydrochloride (Compound 60)

To a mixture of (S)-7-((9,9-dimethyl-9H-fluorene-3-carbonyl)glycyl)-1,4-dioxa-7-azaspiro[4.4]nonane-8-carboxylic acid (50 mg, 0.11 mmol) and 5-(aminomethyl)thiophene-3-carboximidamide (25 mg, 0.16 mmol) in DMF (1.0 mL) was added DIEA (85 mg, 0.66 mmol) and T₃P (41 mg, 0.13 mmol) at 0° C. and the mixture was stirred under N₂ atmosphere at room temperature for 1 hour. The mixture was quenched with saturated aq·NaHCO₃ solution and extracted with EtOAc twice. The combined organic layers were washed with brine, dried over anhydrous Na₂SO₄, filtered, and concentrated under reduced pressure to dryness. The residue was purified by flash chromatography on silica gel (DCM:MeOH=12:1) to give Compound 60 (5.7 mg, yield 8.8%) as a white solid. ¹HNMR (400 MHz, CD₃OD) δ 8.50 (s, 1H), 8.23 (d, J=1.3 Hz, 1H), 8.17 (d, J=1.6 Hz, 1H), 7.81-7.78 (m, 2H), 7.57 (d, J=7.9 Hz, 1H), 7.50 (dd, J=5.9, 2.8 Hz, 1H), 7.42 (d, J=1.4 Hz, 1H), 7.36 (d, J=2.4 Hz, 1H), 7.36-7.34 (m, 1H), 4.65-4.61 (m, 1H), 4.59 (s, 2H), 4.26 (d, J=16.6 Hz, 1H), 4.18 (s, 1H), 4.01-3.98 (m, 4H), 3.82 (s, 2H), 2.44 (dd, J=13.2, 9.2 Hz, 1H), 2.23 (dd, J=13.2, 5.4 Hz, 1H), 1.49 (s, 6H). LCMS (ESI) m/z=588 (M+H)⁺.

### Compound 61: Synthesis of (2S,4R)—N—((R)-1-(4-carbamimidoylthiophen-2-yl)ethyl)-1-((9,9-difluoro-9H-fluorene-3-carbonyl)glycyl)-4-(methylsulfonyl)pyrrolidine-2-carboxamide(Compound 61)

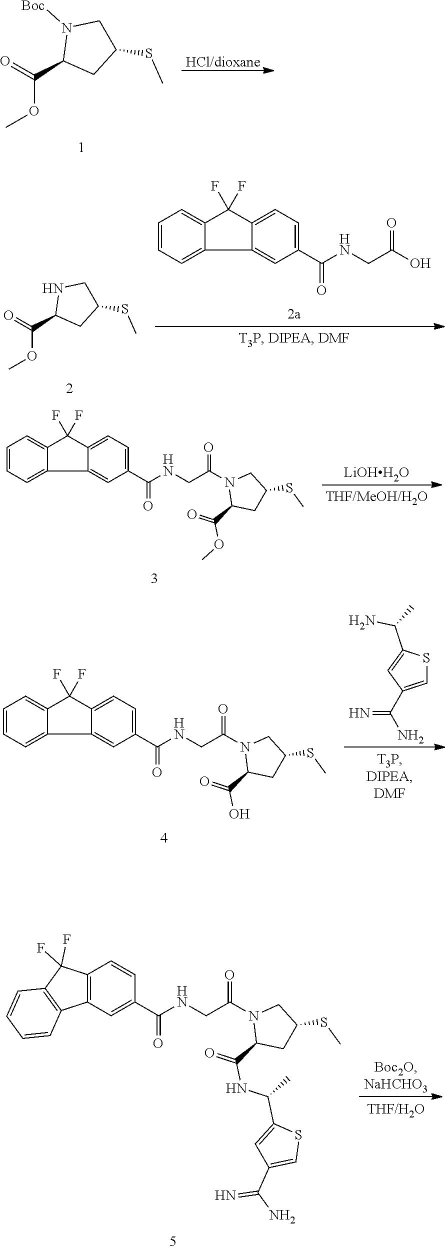

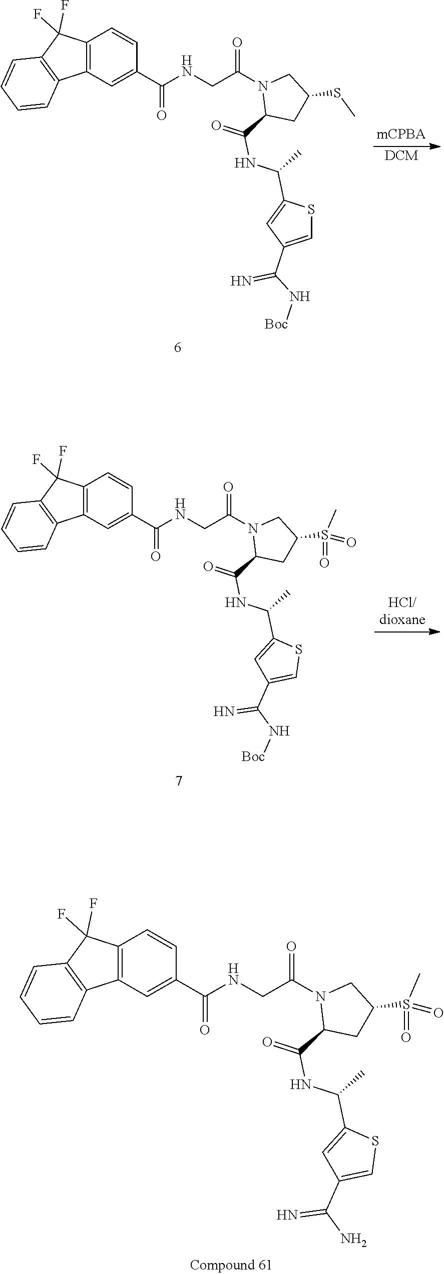

### Step 1: methyl (2S,4R)-4-(methylthio)pyrrolidine-2-carboxylate hydrochloride (2)

To a solution of 1-(tert-butyl) 2-methyl (2S,4R)-4-(methylthio)pyrrolidine-1,2-dicarboxylate (180 mg, 0.65 mmol) in dioxane (2 mL) was added HCl/1,4-dioxane (1 mL) under N₂ atmosphere and the reaction mixture was stirred at room temperature for 2 hours. The mixture was concentrated to dryness under reduced pressure to afford the title compound methyl (2S,4R)-4-(methylthio)pyrrolidine-2-carboxylate (170 mg, yield 100%) as a yellow solid, which was directly used in the next reaction without further purification. LC/MS (ESI) m/z: 176 (M+H)⁺.

### Step 2: methyl (2S,4R)-1-((9,9-difluoro-9H-fluorene-3-carbonyl)glycyl)-4-(methylthio)pyrrolidine-2-carboxylate (3)

To a solution of methyl (2S,4R)-4-(methylthio)pyrrolidine-2-carboxylate hydrochloride (170 mg, 0.65 mmol) and (9,9-difluoro-9H-fluorene-3-carbonyl)glycine (190 mg, 0.63 mmol) in DMF (5 mL) was added DIPEA (487 mg, 3.77 mmol) and T₃P (599 mg, 0.94 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography on silica gel (PE:EtOAc=1:1) to give methyl (2S,4R)-1-((9,9-difluoro-9H-fluorene-3-carbonyl)glycyl)-4-(methylthio)pyrrolidine-2-carboxylate (200 mg, yield 69.2%) as a white solid. LC/MS (ESI) m/z: 461 (M+H)⁺.

### Step 3: (2S,4R)-1-((9,9-difluoro-9H-fluorene-3-carbonyl)glycyl)-4-(methylthio)pyrrolidine-2-carboxylic acid (4)

To a solution of methyl (2S,4R)-1-((9,9-difluoro-9H-fluorene-3-carbonyl)glycyl)-4-(methylthio)pyrrolidine-2-carboxylate (200 mg, 0.43 mmol) in THF/MeOH/H₂O (6 mL, 4/1/1) was added LiOH (36 mg, 0.87 mmol) and the reaction mixture was stirred at room temperature for 2 hours. The mixture was acidified with 1 N aq·HCl to pH ˜4 and extracted with EtOAc twice. The combined organic layers were washed with brine, dried over anhydrous Na₂SO₄, filtered, and concentrated under reduced pressure to dryness to give (2S,4R)-1-((9,9-difluoro-9H-fluorene-3-carbonyl)glycyl)-4-(methylthio)pyrrolidine-2-carboxylic acid (190 mg, yield 98.0%) as a white solid. LC/MS (ESI) m/z: 447 (M+H)⁺.

### Step 4: (2S,4R)—N—((R)-1-(4-carbamimidoylthiophen-2-yl)ethyl)-1-((9,9-difluoro-9H-fluorene-3-carbonyl)glycyl)-4-(methylthio)pyrrolidine-2-carboxamide (5)

To a mixture of (2S,4R)-1-((9,9-difluoro-9H-fluorene-3-carbonyl)glycyl)-4-(methylthio)pyrrolidine-2-carboxylic acid (95 mg, 0.21 mmol) and (R)-5-(1-aminoethyl)thiophene-3-carboximidamide (54 mg, 0.32 mmol) in DMF (5 mL) was added DIPEA (165 mg, 1.28 mmol) and T₃P (203 mg, 0.32 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography on silica gel (PE:EtOAc=3:1) to give (2S,4R)—N—((R)-1-(4-carbamimidoylthiophen-2-yl)ethyl)-1-((9,9-difluoro-9H-fluorene-3-carbonyl)glycyl)-4-(methylthio)pyrrolidine-2-carboxamide (90 mg, yield 70.8%) as a white solid. LC/MS (ESI) m/z: 598 (M+H)⁺.

### Step 5: tert-butyl ((5-((R)-1-((2S,4R)-1-((9,9-difluoro-9H-fluorene-3-carbonyl)glycyl)-4-(methylthio)pyrrolidine-2-carboxamido)ethyl)thiophen-3-yl)(imino)methyl)carbamate (6)

To a solution of (2S,4R)—N—((R)-1-(4-carbamimidoylthiophen-2-yl)ethyl)-1-((9,9-difluoro-9H-fluorene-3-carbonyl)glycyl)-4-(methylthio)pyrrolidine-2-carboxamide (90 mg, 0.15 mmol) in THF/H₂O (5 mL,4/1) was added Boc₂O (66 mg, 0.30 mmol) and NaHCO₃ (63 mg, 0.75 mmol) under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography on silica gel (DCM:MeOH=20:1) to give tert-butyl ((5-((R)-1-((2S,4R)-1-((9,9-difluoro-9H-fluorene-3-carbonyl)glycyl)-4-(methylthio)pyrrolidine-2-carboxamido)ethyl)thiophen-3-yl)(imino)methyl)carbamate (60 mg, yield 57.1%) as a white solid. LC/MS (ESI) m/z: 698 (M+H)⁺.

### Step 6: tert-butyl ((5-((R)-1-((2S,4R)-1-((9,9-difluoro-9H-fluorene-3-carbonyl)glycyl)-4-(methylsulfonyl)pyrrolidine-2-carboxamido)ethyl)thiophen-3-yl)(imino)methyl)carbamate (7)

To a solution of tert-butyl ((5-((R)-1-((2S,4R)-1-((9,9-difluoro-9H-fluorene-3-carbonyl)glycyl)-4-(methylthio)pyrrolidine-2-carboxamido)ethyl)thiophen-3-yl)(imino)methyl)carbamate (60 mg, 0.086 mmol) in DCM (2 ml) was added mCPBA (56 mg, 0.26 mmol) under N₂ atmosphere and the reaction mixture was stirred at room temperature for 3 hours. The mixture was diluted with DCM, washed with water and brine, dried over anhydrous Na₂SO₄, filtered, and concentrated under reduced pressure to dryness. The residue was purified by flash chromatography on silica gel (DCM:MeOH=10:1) to give tert-butyl ((5-((R)-1-((2S,4R)-1-((9,9-difluoro-9H-fluorene-3-carbonyl)glycyl)-4-(methylsulfonyl)pyrrolidine-2-carboxamido)ethyl)thiophen-3-yl)(imino)methyl)carbamate (30 mg, yield 47.8%) as a white solid. LC/MS (ESI) m/z:730 (M+H)⁺.

### Step 7: (2S,4R)—N—((R)-1-(4-carbamimidoylthiophen-2-yl)ethyl)-1-((9,9-difluoro-9H-fluorene-3-carbonyl)glycyl)-4-(methylsulfonyl)pyrrolidine-2-carboxamide (Compound 61)

To a solution of tert-butyl ((5-((R)-1-((2S,4R)-1-((9,9-difluoro-9H-fluorene-3-carbonyl)glycyl)-4-(methylsulfonyl)pyrrolidine-2-carboxamido)ethyl)thiophen-3-yl)(imino)methyl)carbamate (30 mg, 0.041 mmol) in 1,4-dioxane (2 mL) was added HCl/1,4-dioxane (1 mL) under N₂ atmosphere and the reaction mixture was stirred at room temperature for 2 hours. The mixture was concentrated to dryness under reduced pressure. The residue was purified by prep-HPLC to give Compound 61(10 mg, yield 38.6%) as a white solid. ¹H NMR (400 MHz, CD₃OD) δ 8.47 (s, 1H), 8.26-8.14 (m, 2H), 7.86 (dt, J=6.5, 3.3 Hz, 1H), 7.79-7.68 (m, 2H), 7.64 (d, J=7.3 Hz, 1H), 7.60-7.41 (m, 3H), 5.37-5.24 (m, 1H), 4.76 (ddd, J=13.5, 8.6, 4.2 Hz, 1H), 4.36-3.94 (m, 5H), 3.07 (d, J=19.8 Hz, 3H), 2.83 (tdd, J=13.5, 11.0, 7.2 Hz, 1H), 2.61-2.38 (m, 1H), 1.63 (dd, J=33.3, 7.0 Hz, 3H). LC/MS (ESI) m/z: 630 (M+H)⁺.

### Scheme 62: Synthesis of (2S,4R)—N—((R)-1-(4-carbamimidoylthiophen-2-yl)ethyl)-1-((9,9-difluoro-9H-fluorene-3-carbonyl)glycyl)-4-methylpyrrolidine-2-carboxamide (Compound 62)

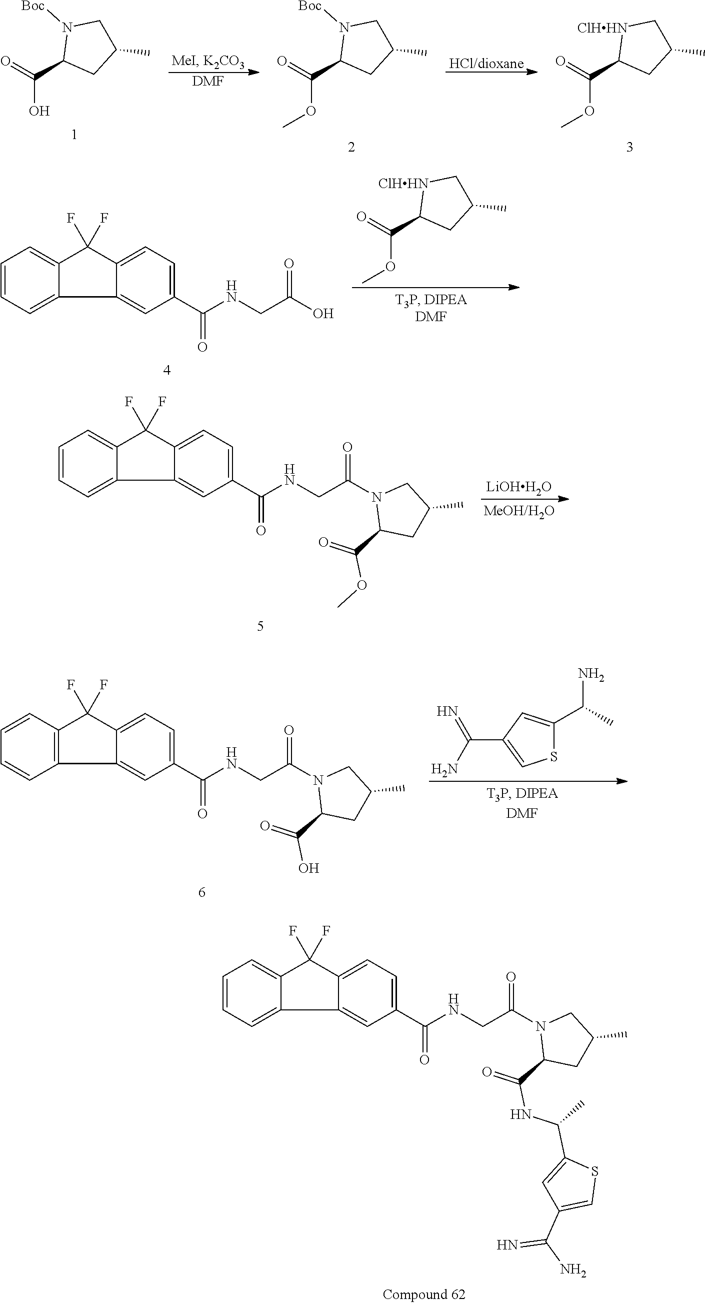

Compound 62 was prepared as a white solid from (2S,4R)-1-(tert-butoxycarbonyl)-4-methylpyrrolidine-2-carboxylic acid (9,9-difluoro-9H-fluorene-3-carbonyl)glycine, and (R)-5-(1-aminoethyl)thiophene-3-based on the procedures set forth in Scheme 46. ¹H NMR (400 MHz, CD₃OD) δ 8.55 (s, 1H), 8.25-8.13 (m, 2H), 7.88 (dt, J=9.0, 4.5 Hz, 1H), 7.77 (t, J=7.9 Hz, 1H), 7.72 (d, J=7.9 Hz, 1H), 7.65 (d, J=7.5 Hz, 1H), 7.58 (t, J=7.5 Hz, 1H), 7.52-7.42 (m, 2H), 5.30 (dq, J=14.3, 7.0 Hz, 1H), 4.63-4.51 (m, 1H), 4.31-4.04 (m, 2H), 3.91 (dt, J=20.0, 10.0 Hz, 1H), 3.25 (t, J=9.2 Hz, 1H), 2.55 (s, 1H), 2.29-2.09 (m, 1H), 2.05-1.84 (m, 1H), 1.62 (dd, J=33.6, 7.0 Hz, 3H), 1.12 (dd, J=13.8, 6.6 Hz, 3H). LC/MS (ESI) m/z: 566 (M+H)⁺.

### Scheme 63: Synthesis of (2S,4R)—N—((R)-1-(4-carbamimidoylthiophen-2-yl)ethyl)-1-((4-(4-fluorophenoxy)benzoyl)glycyl)-4-methylpyrrolidine-2-carboxamide (Compound 63)

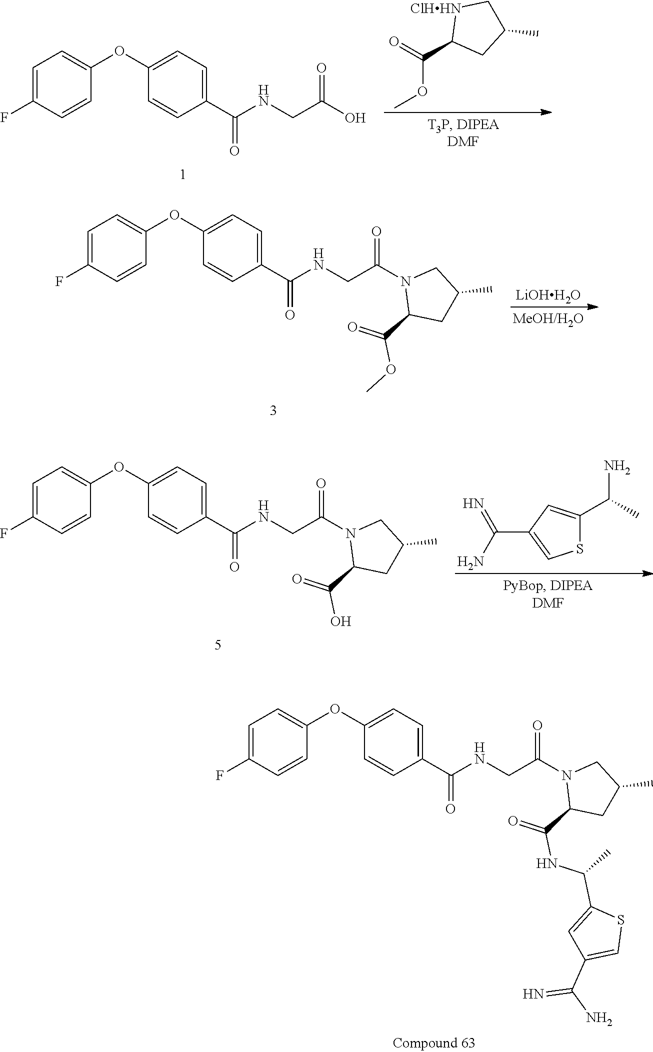

Compound 63 was prepared as a white solid from (4-(4-fluorophenoxy)benzoyl)glycine, methyl (2S,4R)-4-methylpyrrolidine-2-carboxylate hydrochloride, and (R)-5-(1-aminoethyl)thiophene-3-carboximidamide based on the procedures set forth in Scheme 3. ¹H NMR (400 MHz, CD₃OD) δ 8.56 (s, 1H), 8.19 (s, 1H), 7.89-7.84 (m, 2H), 7.50 (d, J=11.0 Hz, 1H), 7.20-7.13 (m, 2H), 7.12-7.07 (m, 2H), 7.02-6.98 (m, 2H), 5.36-5.22 (m, 1H), 4.62-4.52 (m, 1H), 4.26-4.01 (m, 2H), 3.94-3.82 (m, 1H), 3.28-3.12 (m, 1H), 2.55 (dd, J=19.1, 12.6 Hz, 1H), 2.27-2.10 (m, 1H), 2.06-1.85 (m, 1H), 1.60 (t, J=16.0 Hz, 3H), 1.11 (dd, J=12.6, 6.6 Hz, 3H). LC/MS (ESI) m/z: 552 (M+H)⁺.

### Scheme 64: Synthesis of (2S,4R)—N—((R)-1-(4-carbamimidoylthiophen-2-yl)ethyl)-1-((4-(4-cyclopropylphenoxy)benzoyl)glycyl)-4-(difluoromethoxy)pyrrolidine-2-carboxamide hydrochloride (Compound 64)

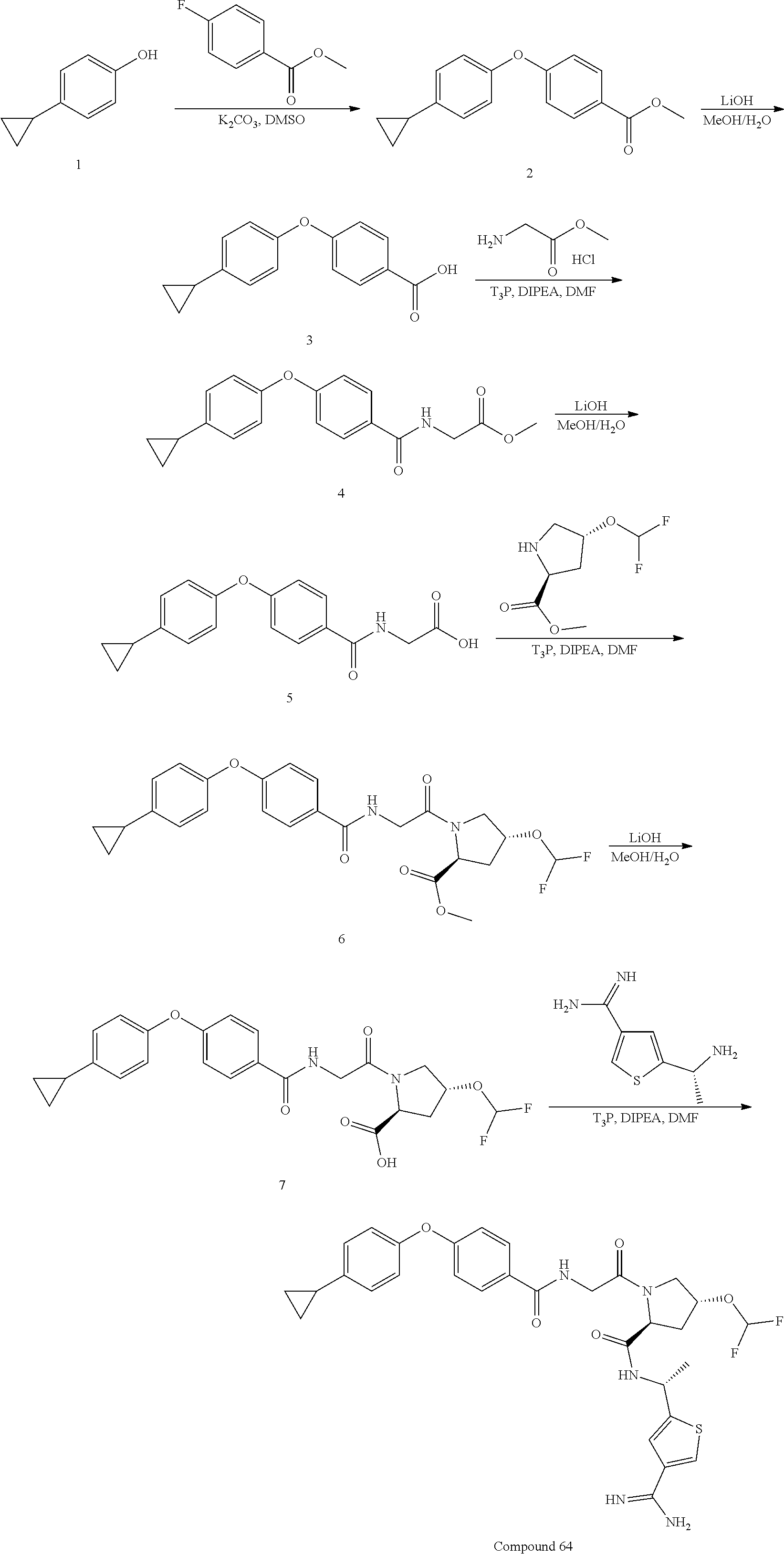

Compound 64 was prepared as a white solid from 4-cyclopropylphenol methyl 4-fluorobenzoate methyl glycinate hydrochloride, methyl (2S,4R)-4-(difluoromethoxy)pyrrolidine-2-carboxylate, and (R)-5-(1-aminoethyl)thiophene-3-carboximidamide based on the procedures set forth in Scheme 24. ¹H NMR (400 MHz, CD₃OD) δ 8.55 (s, 1H), 8.21 (d, J=1.6 Hz, 1H), 7.84-7.80 (m, 2H), 7.54 (d, J=11.1 Hz, 1H), 7.14-7.11 (m, 2H), 6.98-6.93 (m, 4H), 6.50 (dd, J=82.5, 66.4 Hz, 1H), 5.36-5.23 (m, 1H), 5.01 (s, 1H), 4.58 (d, J=7.8 Hz, 1H), 4.19 (dd, J=47.3, 16.7 Hz, 2H), 3.98-3.86 (m, 2H), 2.54-2.42 (m, 1H), 2.26-2.17 (m, 1H), 1.96-1.90 (m, 1H), 1.57 (d, J=7.0 Hz, 3H), 0.98-0.94 (m, 2H), 0.69-0.65 (m, 2H). LC/MS (ESI) (m/z): 626 (M+H)⁺.

### Scheme 65: Synthesis of (2S,4R)—N—((R)-1-(4-carbamimidoylthiophen-2-yl)ethyl)-4-(difluoromethoxy)-1-((4-(4-(difluoromethyl)phenoxy)benzoyl)glycyl)pyrrolidine-2-carboxamide (Compound 65)

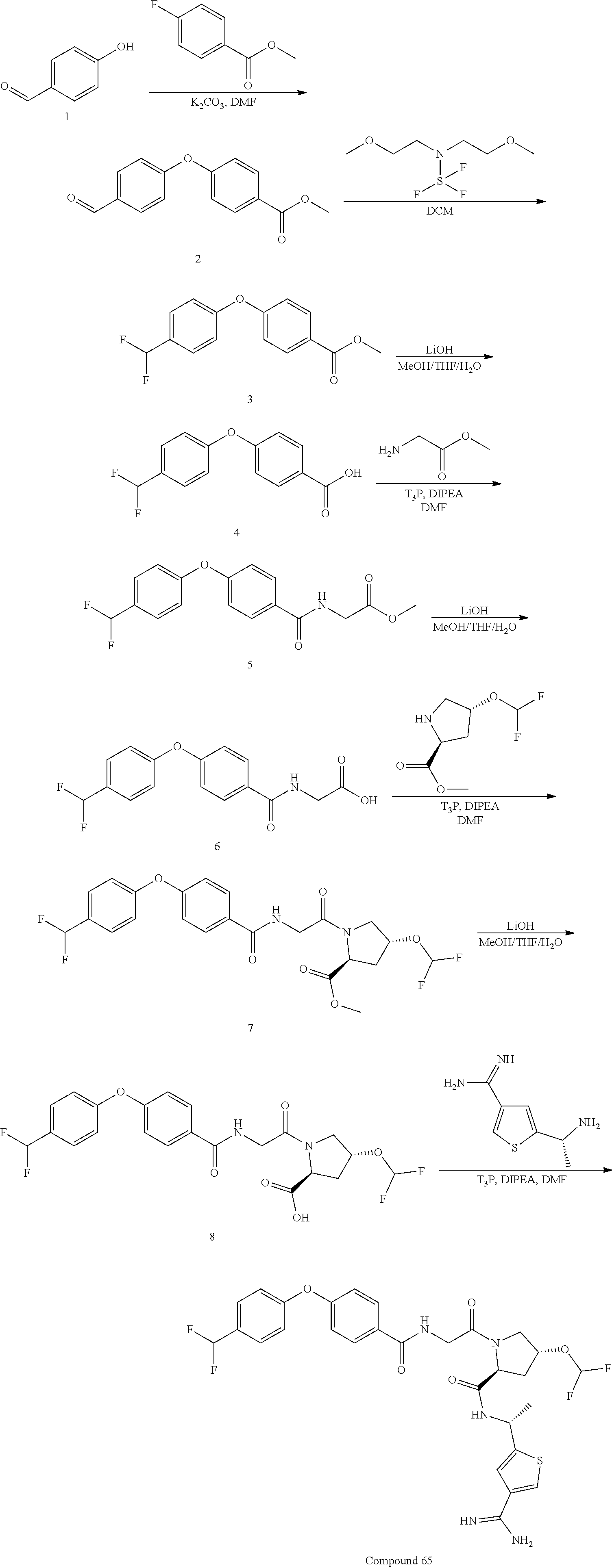

### Step 1: methyl 4-(4-formylphenoxy)benzoate (2)

To a mixture of 4-hydroxybenzaldehyde (2.0 g, 16 mmol) and methyl 4-fluorobenzoate (2.5 g, 16 mmol) in DMSO (20 mL) was added K₂CO₃ (6.7 g, 49 mmol) under N₂ atmosphere and the mixture was stirred at 120° C. overnight. The mixture was diluted with water and extracted with EtOAc twice. The combined organic layers were washed with brine, dried over anhydrous Na₂SO₄, filtered, and concentrated under reduced pressure to dryness. The residue was purified by column chromatography on silica gel (PE:EtOAc=1:1) to give methyl 4-(4-formylphenoxy)benzoate (970 mg, yield 23.1%) as a yellow oil. LCMS (ESI) m/z: 257 (M+H)⁺.

### Step 2: methyl 4-(4-(difluoromethyl)phenoxy)benzoate (3)

To a solution of methyl 4-(4-formylphenoxy)benzoate (970 mg, 3.8 mmol) in DCM (10 mL) was added bis(2-methoxyethyl)aminosulfur trifluoride (837 mg, 3.8 mmol) drop-wisely at OHC under N₂ atmosphere and the mixture was stirred at room temperature overnight. The reaction was quenched with saturated aq·NaHCO₃ solution and extracted with DCM twice. The combined organic layers were washed with brine, dried over anhydrous Na₂SO₄, filtered, and concentrated under reduced pressure to dryness. The residue was purified by column chromatography on silica gel (PE:EtOAc=3:1) to give methyl 4-(4-(difluoromethyl)phenoxy)benzoate (390 mg, yield 36.9%) as a yellow oil. LCMS (ESI) m/z: 279 (M+H)⁺.

### Step 3: 4-(4-(difluoromethyl)phenoxy)benzoic acid (4)

To a solution of methyl 4-(4-(difluoromethyl)phenoxy)benzoate (390 mg, 1.4 mmol) in MeOH (3 mL) and water (1 mL) was added LiOH (344 mg, 8.4 mmol) at 0° C. and the mixture was stirred at room temperature for 2 hours. The mixture was acidified with 1N aq·HCl to pH-3, extracted with EtOAc twice. The combined organic layers were washed with brine, dried over anhydrous Na₂SO₄, filtered, and concentrated under reduced pressure to dryness to give 4-(4-(difluoromethyl)phenoxy)benzoic acid (320 mg, yield 86.5%) as a colorless oil, which was used directly in the next step. LC/MS (ESI) (m/z): 265 (M+H)⁺.

### Step 4: methyl (4-(4-(difluoromethyl)phenoxy)benzoyl)glycinate (5)

To a mixture of 4-(4-(difluoromethyl)phenoxy)benzoic acid (320 mg, 1.2 mmol) and methyl glycinate hydrochloride (152 mg, 1.2 mmol) in DMF (3.5 mL) was added DIPEA (936 mg, 7.3 mmol) and T₃P (496 mg, 1.6 mmol, 50% in EtOAc) at 0° C. and the mixture was stirred under N₂ atmosphere at room temperature for 1 hour. The mixture was quenched with saturated aq·NaHCO₃ solution and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=1:1) to give methyl (4-(4-(difluoromethyl)phenoxy)benzoyl)glycinate (200 mg, yield 49.7%) as a white solid. LCMS (ESI) m/z: 336 (M+H)⁺.

### Step 5: (4-(4-(difluoromethyl)phenoxy)benzoyl)glycine (6)

To a solution of methyl (4-(4-(difluoromethyl)phenoxy)benzoyl)glycinate (200 mg, 1.4 mmol) in MeOH (2 mL) and water (0.7 mL) was added LiOH (148 mg, 3.6 mmol) at 0° C. and the mixture was stirred at room temperature for 2 hours. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (4-(4-(difluoromethyl)phenoxy)benzoyl)glycine (160 mg, yield 83.3%) as a colorless oil, which was used directly in the next step. LC/MS (ESI) (m/z): 322 (M+H)⁺.

### Step 6: methyl (2S,4R)-4-(difluoromethoxy)-1-((4-(4-(difluoromethyl)phenoxy)benzoyl)glycyl)pyrrolidine-2-carboxylate (7)

To a mixture of (4-(4-(difluoromethyl)phenoxy)benzoyl)glycine (110 mg, 0.34 mmol) and methyl (2S,4R)-4-(difluoromethoxy)pyrrolidine-2-carboxylate (67 mg, 0.34 mmol) in DMF (1.5 mL) was added DIPEA (263 mg, 2.0 mmol) and T₃P (140 mg, 0.44 mmol, 50% in EtOAc) at 0° C. and the mixture was stirred under N₂ atmosphere at room temperature for 1 hour. The mixture was quenched with saturated aq·NaHCO₃ solution and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=1:1) to give methyl (2S,4R)-4-(difluoromethoxy)-1-((4-(4-(difluoromethyl)phenoxy)benzoyl)glycyl)pyrrolidine-2-carboxylate (100 mg, yield 59.0%) as a white solid. LCMS (ESI) m/z: 499 (M+H)⁺.

### Step 7: (2S,4R)-4-(difluoromethoxy)-1-((4-(4-(difluoromethyl)phenoxy)benzoyl)glycyl)pyrrolidine-2-carboxylic acid (6)

To a solution of methyl (2S,4R)-4-(difluoromethoxy)-1-((4-(4-(difluoromethyl) phenoxy)benzoyl)glycyl)pyrrolidine-2-carboxylate (100 mg, 1.4 mmol) in MeOH (2 mL) and water (0.7 mL) was added LiOH (49 mg, 1.2 mmol) at 0° C. and the mixture was stirred at room temperature for 2 hours. The mixture was acidified with 1 N aq·HCl to pH-3 and extracted with EtOAc twice. The combined organic layers were washed with brine, dried over anhydrous Na₂SO₄, filtered, and concentrated under reduced pressure to dryness to give (2S,4R)-4-(difluoromethoxy)-1-((4-(4-(difluoromethyl)phenoxy)benzoyl)glycyl)pyrrolidine-2-carboxylic acid (87 mg, yield 90.0%) as a colorless oil, which was used directly in the next step. LC/MS (ESI) (m/z): 485 (M+H)⁺.

### Step 8: (2S,4R)—N—((R)-1-(4-carbamimidoylthiophen-2-yl)ethyl)-4-(difluoromethoxy)-1-((4-(4-(difluoromethyl)phenoxy)benzoyl)glycyl)pyrrolidine-2-carboxamide hydrochloride (Compound 65)

To a mixture of (2S,4R)-4-(difluoromethoxy)-1-((4-(4-(difluoromethyl) phenoxy)benzoyl)glycyl) pyrrolidine-2-carboxylic acid (50 mg, 0.10 mmol) and (R)-5-(1-aminoethyl)thiophene-3-carboximidamide (25 mg, 0.15 mmol) in DMF (1.0 mL) was added DIPEA (77 mg, 0.60 mmol) and T₃P (41 mg, 0.13 mmol, 50% in EtOAc) at 0° C. and the mixture was stirred under nitrogen atmosphere at room temperature for 1 hour. The mixture was quenched with saturated aq·NaHCO₃ solution and extracted with EtOAc twice. The combined organic layers were washed with brine, dried over anhydrous Na₂SO₄, filtered, and concentrated to dryness under reduced pressure. The residue was purified by flash chromatography on silica gel (DCM:MeOH=12:1) and prep-HPLC to give Compound 65 (5.7 mg, yield 9.0%) as a white solid. ¹H NMR (400 MHz, CD₃OD) δ 8.55 (s, 1H), 8.23 (dd, J=10.2, 1.6 Hz, 1H), 7.92-7.87 (m, 2H), 7.60-7.52 (m, 3H), 7.16-7.07 (m, 4H), 6.79 (t, J=41.4 Hz, 1H), 6.49 (dd, J=67.8, 51.4 Hz, 1H), 5.38-5.24 (m, 1H), 5.01 (d, J=3.3 Hz, 1H), 4.58 (t, J=7.8 Hz, 1H), 4.18 (dt, J=44.2, 16.8 Hz, 2H), 3.98-3.86 (m, 2H), 2.52-2.38 (m, 1H), 2.31-2.15 (m, 1H), 1.60 (dd, J=21.5, 8.9 Hz, 3H). LCMS (ESI) m/z: 636 (M+H)⁺.

### Scheme 66: Synthesis of (S)-7-((1,4(1,4)-dibenzenacyclohexaphane-1²-carbonyl)glycyl)-N—((R)-1-(4-carbamimidoylthiophen-2-yl)ethyl)-1,4-dioxa-7-azaspiro[4.4]nonane-8-carboxamide (Compound 66)

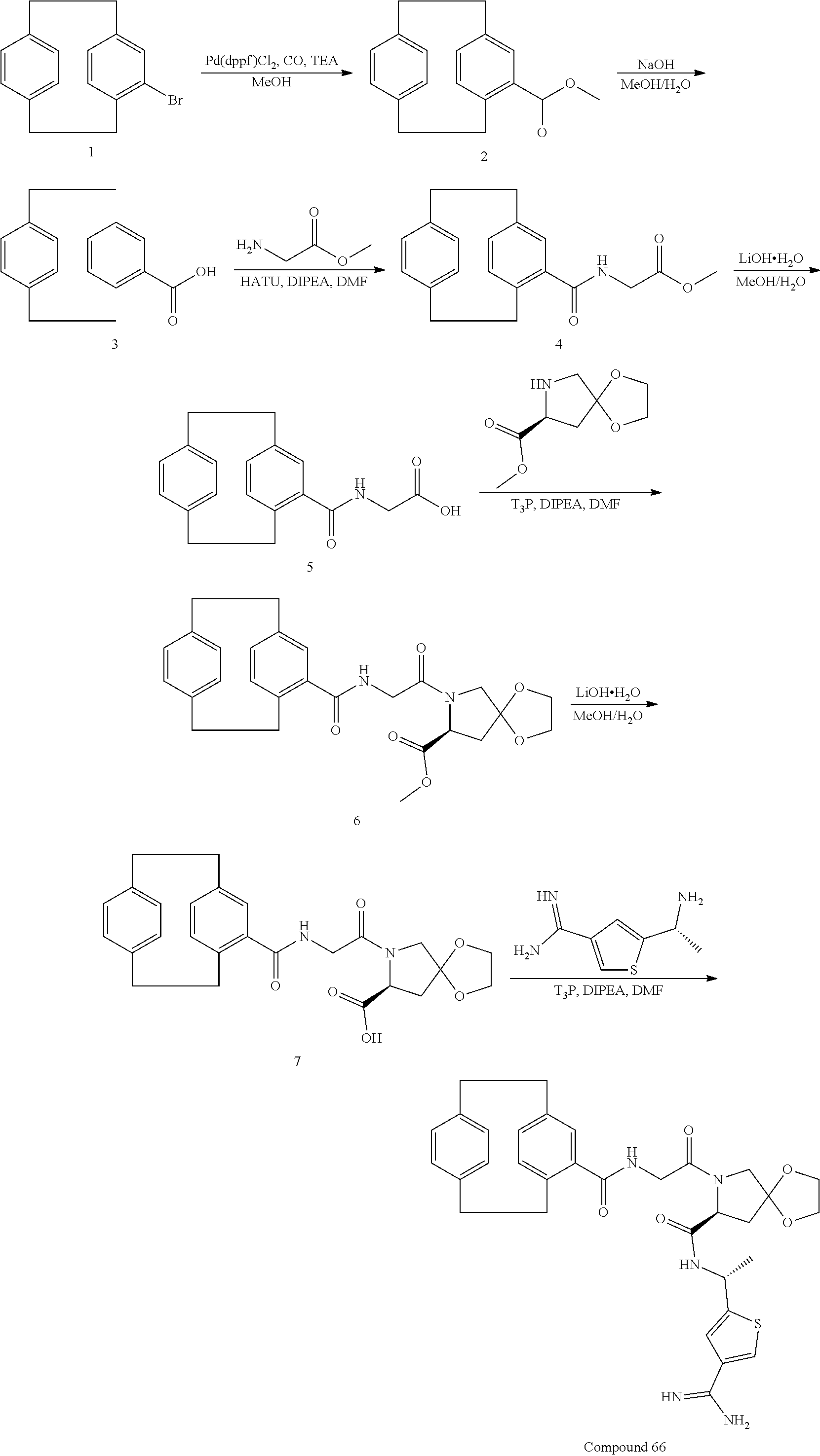

### Step 1: methyl 1,4(1,4)-dibenzenacyclohexaphane-1²-carboxylate (2)

To a mixture of 1²-bromo-1,4(1,4)-dibenzenacyclohexaphane (308 mg, 1.07 mmol) and TEA (0.75 mL, 5.35 mmol) in MeOH (6 mL) was added Pd(dppf)Cl₂ (78 mg, 0.11 mmol) and the mixture was stirred under CO atmosphere at 80° C. for 16 hours. The mixture was diluted with water and extracted with DCM twice. The combined organic layers were dried over anhydrous Na₂SO₄, filtered, and concentrated to dryness under reduced pressure. The residue was purified by column chromatography on silica gel (PE:EtOAc=10:1) to give methyl 1,4(1,4)-dibenzenacyclohexaphane-1²-carboxylate (200 mg, yield 70.2%) as a colorless oil. LC/MS (ESI) m/z: 267 (M+H)⁺.

### Step 2: 1,4(1,4)-dibenzenacyclohexaphane-1²-carboxylic acid (3)

To a solution of methyl 1,4(1,4)-dibenzenacyclohexaphane-1²-carboxylate (180 mg, 0.68 mmol) in MeOH (4 mL) was added a solution of NaOH (541 mg, 13.6 mmol) in water (2 mL) at 0° C., and the mixture was stirred at 70° C. for 3 hours. The mixture was diluted with water and washed with EtOAc twice. The aqueous layer was acidified with 0.5 M aq·HCl to pH˜4 and extracted with EtOAc twice. The combined organic layers were washed with brine, dried over anhydrous Na₂SO₄, filtered, and concentrated to dryness to give 1,4(1,4)-dibenzenacyclohexaphane-1²-carboxylic acid (162 mg, yield 95.3%) as a white solid, which was used directly in the next reaction without further purification. LC/MS (ESI) m/z: 253 (M+H)⁺.

### Step 3: methyl (1,4(1,4)-dibenzenacyclohexaphane-1²-carbonyl)glycinate (4)

To a mixture of 1,4(1,4)-dibenzenacyclohexaphane-1²-carboxylic acid (162 mg, 0.64 mmol) and methyl glycinate (162 mg, 1.28 mmol) in DMF (5 mL) was added DIPEA (0.53 mL, 3.20 mmol) and HATU (366 mg, 0.96 mmol) at 0° C. under N₂ atmosphere and the mixture was stirred at 30° C. for 3 hours. The mixture was diluted with EtOAc, washed with water and brine, dried over anhydrous Na₂SO₄, filtered, and concentrated under reduced pressure to dryness. The residue was purified by flash chromatography (PE:EtOAc=3:1) to give methyl (1,4(1,4)-dibenzenacyclohexaphane-1²-carbonyl)glycinate (180 mg, yield 87.0%) as a colorless oil. LC/MS (ESI) m/z: 324 (M+H)⁺.

### Step 4: (1,4(1,4)-dibenzenacyclohexaphane-1²-carbonyl)glycine (5)

To a solution of methyl (1,4(1,4)-dibenzenacyclohexaphane-1²-carbonyl)glycinate (180 mg, 0.56 mmol) in MeOH (3 mL) was added a solution of LiOH (234 mg, 5.6 mmol) in water (1 mL) at 0° C., and the mixture was stirred at 30° C. for 16 hours. The mixture was diluted with water and washed with EtOAc twice. The aqueous layer was acidified with 0.5 M aq·HCl to pH˜4 and extracted with EtOAc twice. The combined organic layers were washed with brine, dried over anhydrous Na₂SO₄, filtered, and concentrated to dryness to give (1,4(1,4)-dibenzenacyclohexaphane-1²-carbonyl)glycine (160 mg, yield 93.0%) as a white solid, which was used directly in the next reaction without further purification. LC/MS (ESI) m/z: 310 (M+H)⁺.

### Step 5: methyl (S)-7-((1,4(1,4)-dibenzenacyclohexaphane-1²-carbonyl)glycyl)-1,4-dioxa-7-azaspiro[4.4]nonane-8-carboxylate (6)

To a mixture of methyl (S)-1,4-dioxa-7-azaspiro[4.4]nonane-8-carboxylate (60 mg, 0.32 mmol) and (1,4(1,4)-dibenzenacyclohexaphane-1²-carbonyl)glycine (100 mg, 0.32 mmol) in DMF (3 mL) was added DIPEA (0.27 mL, 1.60 mmol) and T₃P (617 mg, 0.96 mmol, 50% wt. in EtOAc) at 0° C. under N₂ atmosphere and the mixture was stirred at 25° C. for 2 hours. The mixture was diluted with EtOAc, washed with water and brine, dried over anhydrous Na₂SO₄, filtered, and concentrated under reduced pressure to dryness. The residue was purified by flash chromatography (PE:EtOAc=3:7) to give methyl (S)-7-((1,4(1,4)-dibenzenacyclohexaphane-1²-carbonyl)glycyl)-1,4-dioxa-7-azaspiro[4.4]nonane-8-carboxylate (143 mg, yield 92.9%) as a white solid. LC/MS (ESI) m/z: 479 (M+H)⁺.

### Step 6: (S)-7-((1,4(1,4)-dibenzenacyclohexaphane-1²-carbonyl)glycyl)-1,4-dioxa-7-azaspiro[4.4]nonane-8-carboxylic acid (7)

To a solution of methyl (S)-7-((1,4(1,4)-dibenzenacyclohexaphane-1²-carbonyl)glycyl)-1,4-dioxa-7-azaspiro[4.4]nonane-8-carboxylate (143 mg, 0.30 mmol) in MeOH (3 mL) was added a solution of LiOH (19 mg, 0.45 mmol) in water (1 mL) at 0° C., and the mixture was stirred at room temperature for 2 hours. The mixture was diluted with water and washed with EtOAc twice. The aqueous layer was acidified with 0.5 M aq·HCl to pH˜4 and extracted with EtOAc twice. The combined organic layers were washed with brine, dried with anhydrous Na₂SO₄, filtered, and concentrated to dryness to give (S)-7-((1,4(1,4)-dibenzenacyclohexaphane-1²-carbonyl)glycyl)-1,4-dioxa-7-azaspiro[4.4]nonane-8-carboxylic acid (120 mg, yield 96.3%) as a white solid, which was used directly in the next reaction without further purification. LC/MS (ESI) m/z: 465 (M+H)⁺.

### Step 7: (S)-7-((1,4(1,4)-dibenzenacyclohexaphane-1²-carbonyl)glycyl)-N—((R)-1-(4-carbamimidoylthiophen-2-yl)ethyl)-1,4-dioxa-7-azaspiro[4.4]nonane-8-carboxamide (Compound 66)

To a mixture of (R)-5-(1-aminoethyl)thiophene-3-carboximidamide HCl salt (59 mg, 0.34 mmol) and (S)-7-((1,4(1,4)-dibenzenacyclohexaphane-1²-carbonyl)glycyl)-1,4-dioxa-7-azaspiro[4.4]nonane-8-carboxylic acid (80 mg, 0.17 mmol) in DMF (3 mL) was added DIPEA (0.14 mL, 0.85 mmol) and T₃P (164 mg, 0.26 mmol, 50% wt. in EtOAc) at 0° C. under N₂ atmosphere and the mixture was stirred at 25° C. for 3 hours. The mixture was quenched with saturated aq·NaHCO₃ solution and extracted with CHCl₃/i-PrOH (3/1, v/v) twice. The combined organic layers were washed with brine, dried over anhydrous Na₂SO₄, filtered, and concentrated under reduced pressure to dryness. The residue was purified by prep-TLC (DCM:MeOH=10:1) and further purified by prep-HPLC to give Compound 66 (20 mg, yield 18.9%) as a white solid. ¹HNMR (400 MHz, CD₃OD) δ 8.55 (s, 1H), 8.29-8.18 (m, 1H), 7.53 (t, J=7.8 Hz, 1H), 6.79 (dd, J=3.9, 1.7 Hz, 1H), 6.73-6.63 (m, 2H), 6.55 (d, J=8.0 Hz, 3H), 6.48-6.42 (m, 1H), 5.34 (tt, J=12.8, 6.6 Hz, 1H), 4.59 (dd, J=8.7, 6.6 Hz, 1H), 4.22-4.07 (m, 2H), 4.05-3.97 (m, 4H), 3.81 (d, J=10.6 Hz, 1H), 3.78 (d, J=4.9 Hz, 1H), 3.24-2.83 (m, 8H), 2.48-2.20 (m, 2H), 1.66 (ddd, J=15.6, 7.8, 5.4 Hz, 3H). LC/MS (ESI) (m/z): 616 (M+H)⁺.

### Scheme 67: Synthesis of (1S,3S,5S)—N—((R)-1-(5-carbamimidoylthiophen-2-yl)ethyl)-2-((4-(4-fluorophenoxy)benzoyl)glycyl)-5-methyl-2-azabicyclo[3.1.0]hexane-3-carboxamide (Compound 67)

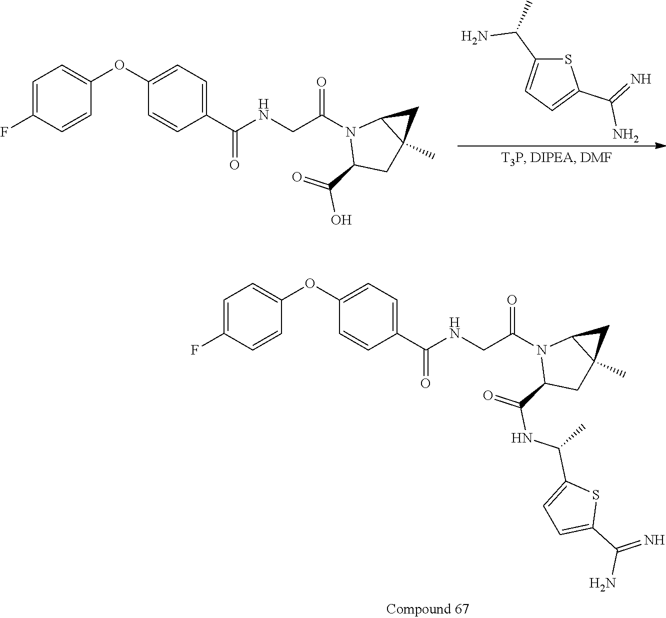 [SMILES: C[C@@]12C[C@@H]1N(C(=O)CNC(=O)c1ccc(Oc3ccc(F)cc3)cc1)[C@H](C(=O)O)C2 ; C[C@@H](N)c1ccc(C(=N)N)s1 ; C[C@@H](NC(=O)[C@@H]1C[C@]2(C)C[C@@H]2N1C(=O)CNC(=O)c1ccc(Oc2ccc(F)cc2)cc1)c1ccc(C(=N)N)s1]

Compound 67 was prepared as a white solid from (1S,3S,5S)-2-((4-(4-fluorophenoxy)benzoyl)glycyl)-5-methyl-2-azabicyclo[3.1.0]hexane-3-carboxylic acid and (R)-5-(1-aminoethyl)thiophene-2-carboximidamide based on the procedures set forth in Scheme 3. ¹H NMR (400 MHz, CD₃OD) δ 8.45 (s, 1H), 7.81 (d, J=8.8 Hz, 2H), 7.72 (d, J=4.0 Hz, 1H), 7.14-7.08 (m, 3H), 7.05-7.01 (m, 2H), 6.95-6.92 (m, 2H), 5.20 (q, J=7.0 Hz, 1H), 4.78-4.75 (m, 1H), 4.39 (d, J=16.6 Hz, 1H), 4.18 (d, J=16.5 Hz, 1H), 3.34 (dd, J=5.9, 2.3 Hz, 1H), 2.38 (t, J=12.6 Hz, 1H), 2.09 (dd, J=13.4, 3.5 Hz, 1H), 1.52 (d, J=7.0 Hz, 3H), 1.25 (s, 3H), 1.18 (dd, J=5.4, 2.5 Hz, 1H), 0.73 (t, J=5.7 Hz, 1H). LC/MS (ESI) m/z: 564 (M+H)⁺.

### Scheme 68: Synthesis of (2S,4R)—N-((5-carbamimidoylthiophen-2-yl)methyl)-4-fluoro-1-((4-(4-fluorophenoxy)benzoyl)glycyl)-4-(methoxymethyl)pyrrolidine-2-carboxamide (Compound 68)

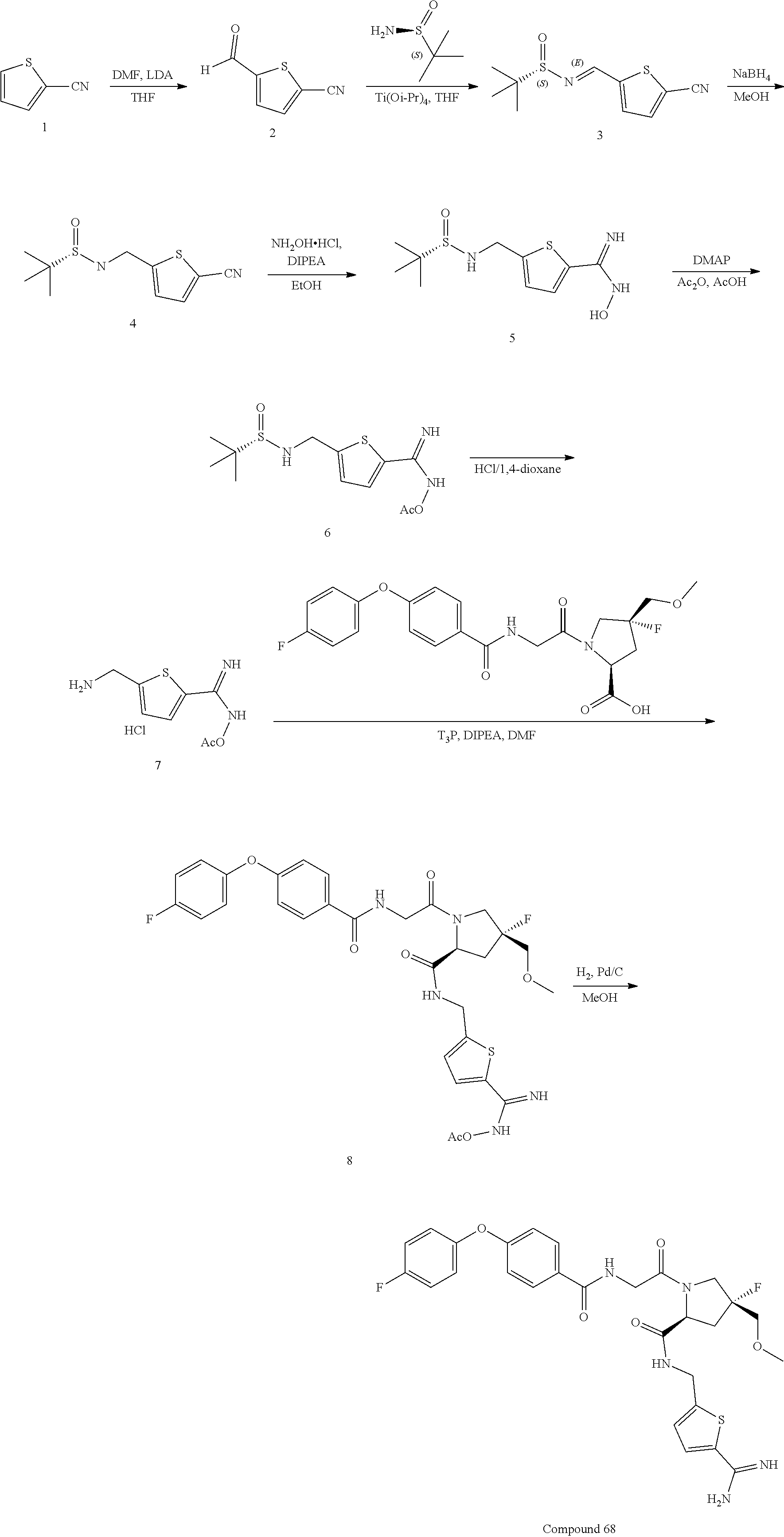

### Step 1: 5-formylthiophene-2-carbonitrile (2)

At −70° C., to a solution of thiophene-2-carbonitrile (2.0 g, 18.34 mmol) in THF (20 mL) was added LDA (10.09 mL, 20.18 mmol) drop-wisely at −70° C. and the mixture was stirred at this temperature for 45 minutes. Then DMF (5.43 g, 73.36 mmol) was added at −70° C. and the resulting mixture was stirred at −70° C. for 2 hours. The mixture was quenched with ice-water and extracted with EtOAc twice. The combined organic layers were washed with brine, dried over anhydrous Na₂SO₄, filtered, and concentrated to dryness. The residue was purified by chromatography on silica gel (PE:EtOAc=80:1) to give 5-formylthiophene-2-carbonitrile (400 mg, 15.9% yield) as a yellow solid.

### Step 2: (S,E)-N-((5-cyanothiophen-2-yl)methylene)-2-methylpropane-2-sulfinamide (3)

To a solution of 5-formylthiophene-2-carbonitrile (400 mg, 2.92 mmol) in THF (6 mL) was added (S)-2-methylpropane-2-sulfinamide (389 mg, 3.21 mmol) and titanium tetraisopropanolate (1.66 g, 5.84 mmol) at 0° C. and the reaction mixture was stirred at room temperature for 16 hours. The mixture was quenched with ice-water and extracted with DCM twice. The combined organic layers were washed with brine, dried over anhydrous Na₂SO₄, filtered, and concentrated to dryness. The residue was purified by chromatography on silica gel (PE:EtOAc=55:1) to give (S,E)-N-((5-cyanothiophen-2-yl)methylene)-2-methylpropane-2-sulfinamide (460 mg, yield 65.7%) as a light-yellow. LC/MS (ESI) (m/z): 241(M+H)⁺.

### Step 3: (S)—N-((5-cyanothiophen-2-yl)methyl)-2-methylpropane-2-sulfinamide (4)

To a mixture of (S,E)-N-((5-cyanothiophen-2-yl)methylene)-2-methylpropane-2-sulfinamide (460 mg, 1.93 mmol) in MeOH (6 mL) was added NaBH₄ (88 mg, 2.31 mmol) at 0° C. and the reaction mixture was stirred at 0° C. for 2 hours. After completion of the reaction, the reaction mixture was quenched with ice water and extracted with DCM twice. The combined organic layers were washed with brine, dried, and concentrated to dryness. The residue was purified by chromatography on silica gel (PE:EtOAc=1:1) to give (S)—N-((5-cyanothiophen-2-yl)methyl)-2-methylpropane-2-sulfinamide (460 mg, yield 99.1%) as a light oil. LC/MS (ESI) m/z: 243 (M+H)⁺.

### Step 4: (S)-5-(((tert-butylsulfinyl)amino)methyl)-N-hydroxythiophene-2-carboximidamide (5)

To a solution of (S)—N-((5-cyanothiophen-2-yl)methyl)-2-methylpropane-2-sulfinamide (460 mg, 1.95 mmol) in EtOH (6 mL) was added NH₂OH·HCl (334 mg, 4.80 mmol) and DIPEA (743 mg, 5.76 mmol) and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography on silica gel (PE:EtOAc=1:1) to give (S)-5-(((tert-butylsulfinyl)amino)methyl)-N-hydroxythiophene-2-carboximidamide (460 mg, yield 88.1%) as a white solid. LC/MS (ESI) m/z: 276 (M+H)⁺.

### Step 5: (S)—N-acetoxy-5-(((tert-butylsulfinyl)amino)methyl)thiophene-2-carboximidamide (6)

To a solution of (S)-5-(((tert-butylsulfinyl)amino)methyl)-N-hydroxythiophene-2-carboximidamide (460 mg, 1.67 mmol) in AcOH (3 mL) was added DMAP (61 mg, 0.50 mmol) and Ac₂O (3 mL). The mixture was stirred at room temperature overnight. The mixture was concentrated under reduce pressure and the residue was purified by chromatography on silica gel (DCM:MeOH=80:1) to give (S)—N-acetoxy-5-(((tert-butylsulfinyl)amino)methyl)thiophene-2-carboximidamide (170 mg, yield 32.1%) as a white solid. LC/MS (ESI) m/z: 318 (M+H)⁺.

### Step 6: N-acetoxy-5-(aminomethyl)thiophene-2-carboximidamide hydrochloride (7)

A solution of (S)—N-acetoxy-5-(((tert-butylsulfinyl)amino)methyl)thiophene-2-carboximidamide (55 mg, 0.17 mmol) in HCl/1,4-dioxane (3 mL) was stirred at room temperature for 2 hours. The mixture was concentrated to dryness under reduced pressure to give methyl (2S,4R)-4-(methylthio)pyrrolidine-2-carboxylate hydrochloride (45 mg, 100% yield) as a white solid, which was directly used in the next reaction without further purification. LC/MS (ESI) m/z: 214 (M+H)⁺.

### Step 7: (2S,4R)—N-((5-(N-acetoxycarbamimidoyl)thiophen-2-yl)methyl)-4-fluoro-1-((4-(4-fluorophenoxy)benzoyl)glycyl)-4-(methoxymethyl)pyrrolidine-2-carboxamide (6)

To a mixture of methyl (2S,4R)-4-(methylthio)pyrrolidine-2-carboxylate (45 mg, 0.17 mmol) and (2S,4R)-4-fluoro-1-((4-(4-fluorophenoxy)benzoyl)glycyl)-4-(methoxymethyl)pyrrolidine-2-carboxylic acid (76 mg, 0.17 mmol) in DMF (2 mL) was added DIPEA (0.14 mL, 0.85 mmol) and PyBOP (132 mg, 0.26 mmol) at 0° C. and the mixture was stirred at room temperature for 1 hour. The mixture was diluted with water and extracted with CHCl₃/i-PrOH (v/v=3/1) twice. The combined organic layers were washed with brine, dried over anhydrous Na₂SO₄, filtered, and concentrated under reduced pressure to dryness. The residue was purified by prep-TLC (DCM:MeOH=15:1) to give (2S,4R)—N-((5-(N-acetoxycarbamimidoyl)thiophen-2-yl)methyl)-4-fluoro-1-((4-(4-fluorophenoxy)benzoyl)glycyl)-4-(methoxymethyl)pyrrolidine-2-carboxamide (80 mg, yield 73.7%) as a white solid. LC/MS(ESI) m/z: 644 (M+H)⁺.

### Step 8: (2S,4R)—N-((5-carbamimidoylthiophen-2-yl)methyl)-4-fluoro-1-((4-(4-fluorophenoxy) benzoyl)glycyl)-4-(methoxymethyl)pyrrolidine-2-carboxamide (Compound 66)

To a solution of (2S,4R)—N-((5-(N-acetoxycarbamimidoyl)thiophen-2-yl)methyl)-4-fluoro-1-((4-(4-fluorophenoxy)benzoyl)glycyl)-4-(methoxymethyl)pyrrolidine-2-carboxamide (80 mg, 0.12 mmol) in MeOH (3 mL) was added Pd/C (20 mg, 10% wt.) under N₂ atmosphere and the reaction mixture was stirred at 25° C. under a H₂ balloon for 2 hours. The mixture was filtered, and the filtrate was concentrated under reduced pressure to dryness. The residue was purified by prep-HPLC to give Compound 68 (20 mg, yield 27.5%) as a white solid. ¹H NMR (400 MHz, CD₃OD) δ 8.52 (s, 1H), 7.88-7.82 (m, 2H), 7.81-7.73 (m, 1H), 7.24-7.12 (m, 3H), 7.12-7.05 (m, 2H), 7.02-6.94 (m, 2H), 4.77-4.57 (m, 3H), 4.30-3.83 (m, 4H), 3.73-3.63 (m, 2H), 3.42 (d, J=8.4 Hz, 3H), 2.80-2.48 (m, 1H), 2.40-2.10 (m, 1H). LC/MS (ESI) m/z: 586 (M+H)⁺.

### Scheme 69: Synthesis of (1S,3S,5R)-5-((2-aminoethoxy)methyl)-N—((R)-1-(4-carbamimidoylthiophen-2-yl)ethyl)-2-((4-(4-fluorophenoxy)benzoyl)glycyl)-2-azabicyclo[3.1.0]hexane-3-carboxamide (Compound 69)

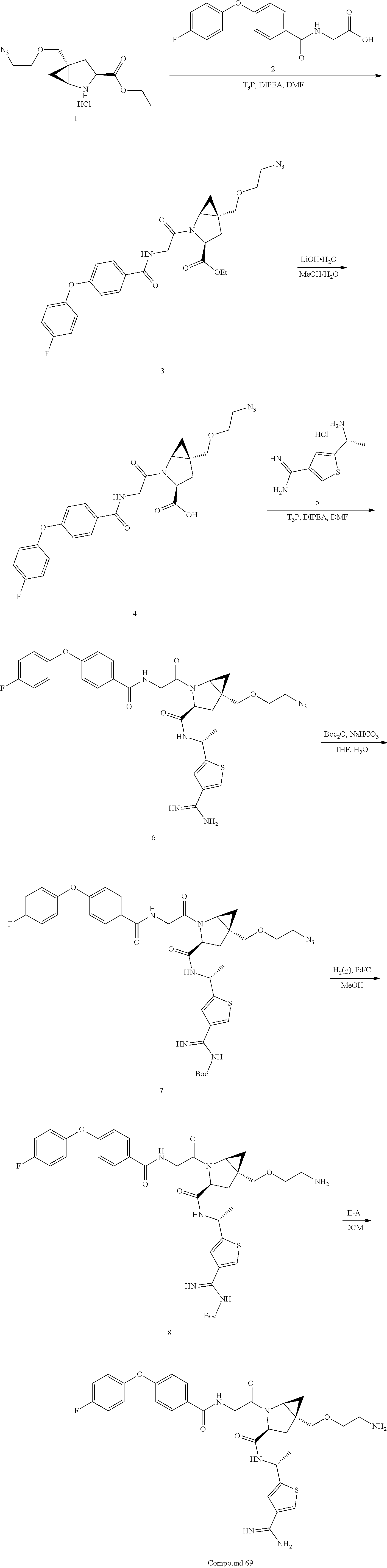

### Step 1: ethyl (1S,3S,5R)-5-((2-azidoethoxy)methyl)-2-((4-(4-fluorophenoxy)benzoyl)glycyl)-2-azabicyclo[3.1.0]hexane-3-carboxylate (3

To a mixture of ethyl (1S,3S,5R)-5-((2-azidoethoxy)methyl)-2-azabicyclo[3.1.0]hexane-3-carboxylate hydrochloride (718 mg, 2.83 mmol) and (4-(4-fluorophenoxy)benzoyl)glycine (899 mg, 3.11 mmol) in DMF (20 mL) was added DIPEA (2.3 mL, 14.15 mmol) and T₃P (5.4 g, 8.49 mmol, 50% wt. in EtOAc) at 0° C. under N₂ atmosphere and the mixture was stirred at 28° C. for 2 hours. The mixture was diluted with EtOAc, washed with water and brine, dried over anhydrous Na₂SO₄, filtered, and concentrated under reduced pressure to dryness. The residue was purified by flash chromatography (DCM:MeOH=40:1) to give ethyl (1S,3S,5R)-5-((2-azidoethoxy)methyl)-2-((4-(4-fluorophenoxy)benzoyl)glycyl)-2-azabicyclo[3.1.0]hexane-3-carboxylate (1.3 g, yield 87.8%) as a brown semi-solid. LC/MS (ESI) m/z: 526 (M+H)⁺.

### Step 2: (1S,3S,5R)-5-((2-azidoethoxy)methyl)-2-((4-(4-fluorophenoxy)benzoyl)glycyl)-2-azabicyclo[3.1.0]hexane-3-carboxylic acid (4)

To a solution of ethyl (1 S,3S,5R)-5-((2-azidoethoxy)methyl)-2-((4-(4-fluorophenoxy)benzoyl)glycyl)-2-azabicyclo[3.1.0]hexane-3-carboxylate (1.34 g, 2.55 mmol) in MeOH (12 mL) was added a solution of LiOH (160 mg, 3.83 mmol) in water (4 mL) at 0° C., and the mixture was stirred at 25° C. for 2 hours. The mixture was diluted with water and washed with EtOAc twice. The aqueous layer was acidified with 0.5 M aq·HCl to pH˜4 and extracted with EtOAc twice. The combined organic layers were washed with brine, dried over anhydrous Na₂SO₄, filtered, and concentrated to dryness to give (1 S,3S,5R)-5-((2-azidoethoxy)methyl)-2-((4-(4-fluorophenoxy)benzoyl)glycyl)-2-azabicyclo[3.1.0]hexane-3-carboxylic acid (1.2 g, yield 94.4%) as a yellow solid, which was used directly in the next reaction without further purification. LC/MS (ESI) m/z: 498 (M+H)⁺.

### Step 3: (1S,3S,5R)-5-((2-azidoethoxy)methyl)-N—((R)-1-(4-carbamimidoylthiophen-2-yl)ethyl)-2-((4-(4-fluorophenoxy)benzoyl)glycyl)-2-azabicyclo[3.1.0]hexane-3-carboxamide (6)

To a mixture of (R)-5-(1-aminoethyl)thiophene-3-carboximidamide hydrochloride (410 mg, 2.42 mmol) and (1S,3S,5R)-5-((2-azidoethoxy)methyl)-2-((4-(4-fluorophenoxy)benzoyl)glycyl)-2-azabicyclo[3.1.0]hexane-3-carboxylic acid (600 mg, 1.21 mmol) in DMF (10 mL) was added DIPEA (1.7 mL, 6.05 mmol) and T₃P (2.3 g, 3.63 mmol, 50% wt. in EtOAc) at 0° C. under N₂ atmosphere and the mixture was stirred at 28° C. for 3 hours. The mixture was quenched with saturated aq·NaHCO₃ solution and extracted with CHCl₃/i-PrOH (3/1, v/v) twice. The combined organic layers were washed with brine, dried over anhydrous Na₂SO₄, filtered, and concentrated under reduced pressure to dryness to give (1S,3S,5R)-5-((2-azidoethoxy)methyl)-N—((R)-1-(4-carbamimidoylthiophen-2-yl)ethyl)-2-((4-(4-fluorophenoxy)benzoyl)glycyl)-2-azabicyclo[3.1.0]hexane-3-carboxamide (780 mg, yield 99.7%) as a brown oil. LC/MS (ESI) m/z: 649 (M+H)⁺.

### Step 4: tert-butyl ((5-((R)-1-((1S,3S,5R)-5-((2-azidoethoxy)methyl)-2-((4-(4-fluorophenoxy)benzoyl)glycyl)-2-azabicyclo[3.1.0]hexane-3-carboxamido)ethyl)thiophen-3-yl)(imino)methyl)carbamate (7)

To a mixture of (1S,3S,5R)-5-((2-azidoethoxy)methyl)-N—((R)-1-(4-carbamimidoylthiophen-2-yl)ethyl)-2-((4-(4-fluorophenoxy)benzoyl)glycyl)-2-azabicyclo[3.1.0]hexane-3-carboxamide (800 mg, 1.23 mmol) and di-tert-butyl dicarbonate (837 mg, 3.69 mmol) in THF (16 mL) was added a solution of NaHCO₃ (1.07 g, 12.3 mmol) in H₂O (16 mL) drop-wisely at 0° C. and the mixture was stirred at 25° C. for 16 hours. The mixture was diluted with water and extracted with EtOAc twice. The combined organic layers were dried over anhydrous Na₂SO₄, filtered, and concentrated under reduced pressure to dryness. The residue was purified by flash chromatography (DCM:MeOH=20:1) to give tert-butyl ((5-((R)-1-((1 S,3S,5R)-5-((2-azidoethoxy)methyl)-2-((4-(4-fluorophenoxy)benzoyl)glycyl)-2-azabicyclo[3.1.0]hexane-3-carboxamido)ethyl)thiophen-3-yl)(imino)methyl)carbamate (268 mg, yield 29.0%) as a yellow solid. LC/MS (ESI) m/z: 749 (M+H)⁺.

### Step 5: tert-butyl ((5-((R)-1-((1S,3S,5R)-5-((2-aminoethoxy)methyl)-2-((4-(4-fluorophenoxy)benzoyl)glycyl)-2-azabicyclo[3.1.0]hexane-3-carboxamido)ethyl)thiophen-3-yl)(imino)methyl)carbamate (6)

To a solution of tert-butyl ((5-((R)-1-((1 S,3S,5R)-5-((2-azidoethoxy)methyl)-2-((4-(4-fluorophenoxy)benzoyl)glycyl)-2-azabicyclo[3.1.0]hexane-3-carboxamido)ethyl)thiophen-3-yl)(imino)methyl)carbamate (30 mg, 0.04 mmol) in MeOH (5 mL) was added Pd/C (10 mg, 10% wt.) at 0° C., and the mixture was degassed under N₂ atmosphere for three times and stirred under a H₂ balloon at room temperature overnight. The mixture was filtered, and the filtrate was concentrated under reduce pressure to give tert-butyl ((5-((R)-1-((1 S,3S,5R)-5-((2-aminoethoxy)methyl)-2-((4-(4-fluorophenoxy)benzoyl)glycyl)-2-azabicyclo[3.1.0]hexane-3-carboxamido)ethyl)thiophen-3-yl)(imino)methyl)carbamate (28 mg, yield 96.6%) as a white solid, which was used directly in the next reaction without further purification. LC/MS (ESI) (m/z): 723 (M+H)⁺.

### Step 6: (1S,3S,5R)-5-((2-aminoethoxy)methyl)-N—((R)-1-(4-carbamimidoylthiophen-2-yl)ethyl)-2-((4-(4-fluorophenoxy)benzoyl)glycyl)-2-azabicyclo[3.1.0]hexane-3-carboxamide (Compound 69)

A solution of tert-butyl ((5-((R)-1-((1S,3S,5R)-5-((2-aminoethoxy)methyl)-2-((4-(4-fluorophenoxy)benzoyl)glycyl)-2-azabicyclo[3.1.0]hexane-3-carboxamido)ethyl)thiophen-3-yl)(imino)methyl)carbamate (28 mg, 0.04 mmol) in DCM (2 mL) was added TFA (1 mL) and the mixture was stirred at room temperature for 1 hour. The reaction mixture was concentrated to dryness under reduced pressure, co-evaporated with DCM and dried under vacuum. The residue was purified by prep-HPLC to give Compound 69 (5 mg, yield 20.8%) as a white solid. ¹H NMR (400 MHz, CD₃OD) δ 8.51 (s, 2H), 8.23 (t, J=3.7 Hz, 1H), 7.88-7.83 (m, 2H), 7.54-7.48 (m, 1H), 7.20-7.14 (m, 2H), 7.09 (ddd, J=6.8, 5.3, 3.0 Hz, 2H), 7.02-6.98 (m, 2H), 5.32-5.19 (m, 1H), 4.88 (s, 1H), 4.45 (d, J=16.5 Hz, 1H), 4.22 (d, J=16.4 Hz, 1H), 3.67 (ddd, J=8.6, 8.0, 4.0 Hz, 4H), 3.54 (d, J=10.5 Hz, 1H), 3.17-3.12 (m, 2H), 2.71 (t, J=12.5 Hz, 1H), 2.15 (dd, J=13.4, 3.6 Hz, 1H), 1.61 (dd, J=24.8, 7.0 Hz, 3H), 1.36 (dd, J=5.7, 2.7 Hz, 1H), 1.03 (t, J=5.9 Hz, 1H). LC/MS (ESI) (m/z): 623 (M+H)⁺.

### Scheme 70: Synthesis of (2S,4R)—N—((R)-1-(4-carbamimidoylthiophen-2-yl)ethyl)-1-((4-(4-fluorophenoxy)benzoyl)glycyl)-4-methoxypyrrolidine-2-carboxamide (Compound 70)

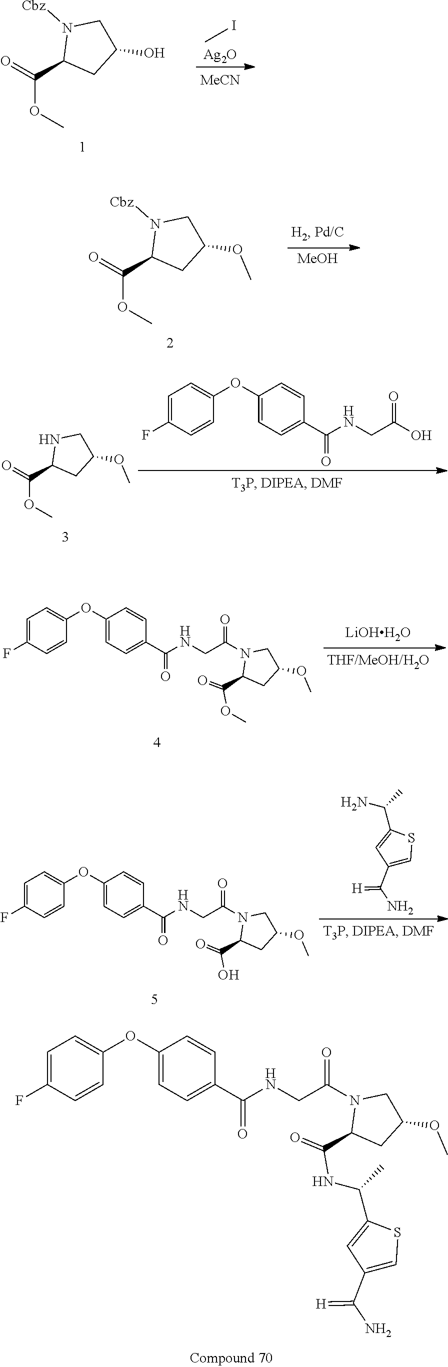

### Step 1: 1-benzyl 2-methyl (2S,4R)-4-methoxypyrrolidine-1,2-dicarboxylate (2)

To a solution of 1-benzyl 2-methyl (2S,4R)-4-hydroxypyrrolidine-1,2-dicarboxylate (5 g, 17.90 mmol) in MeCN (50 ml) was added MeI (7.6 g, 53.71 mmol) and Ag₂O (4.1 g, 17.90 mmol) under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography on silica gel (PE:EtOAc=10:1) to give 1-benzyl 2-methyl (2S,4R)-4-methoxypyrrolidine-1,2-dicarboxylate (1.85 g, yield 35.2%) as a white solid. LC/MS (ESI) m/z: 294 (M+H)⁺.

### Step 2: methyl (2S,4R)-4-methoxypyrrolidine-2-carboxylate (3)

To a solution of 1-benzyl 2-methyl (2S,4R)-4-methoxypyrrolidine-1,2-dicarboxylate (500 mg, 1.70 mmol) in MeOH (3 mL) was added Pd/C (30 mg, 10% wt) and the reaction mixture was stirred under a H₂ balloon at room temperature overnight. The mixture was filtered, and the filtrate was concentrated under reduced pressure to dryness. The residue was purified by prep-TLC (DCM:MeOH=10:1) to give methyl (2S,4R)-4-methoxypyrrolidine-2-carboxylate (270 mg, yield 99.5%) as a white solid. LC/MS (ESI) m/z: 160 (M+H)⁺.

### Step 3: methyl (2S,4R)-1-((4-(4-fluorophenoxy)benzoyl)glycyl)-4-methoxypyrrolidine-2-carboxylate (4)

To a mixture of methyl (2S,4R)-4-methoxypyrrolidine-2-carboxylate (135 mg, 0.85 mmol) and (4-(4-fluorophenoxy)benzoyl)glycine (245 mg, 0.85 mmol) in DMF (5 mL) was added DIPEA (658 mg, 5.09 mmol) and T₃P (809 mg, 1.27 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography on silica gel (PE:EtOAc=1:2) to give methyl (2S,4R)-1-((4-(4-fluorophenoxy)benzoyl)glycyl)-4-methoxypyrrolidine-2-carboxylate (300 mg, yield 82.2%) as a white solid. LC/MS (ESI) m/z: 431 (M+H)⁺.

### Step 4: (2S,4R)-1-((4-(4-fluorophenoxy)benzoyl)glycyl)-4-methoxypyrrolidine-2-carboxylic acid (5)

To a solution of methyl (2S,4R)-1-((4-(4-fluorophenoxy)benzoyl)glycyl)-4-methoxypyrrolidine-2-carboxylate (300 mg, 0.70 mmol) in THF/MeOH/H₂O (6 mL,4/1/1) was added LiOH (59 mg, 1.39 mmol) and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography on silica gel (DCM:MeOH=10:1) to give (2S,4R)-1-((4-(4-fluorophenoxy)benzoyl)glycyl)-4-methoxypyrrolidine-2-carboxylic acid (290 mg, yield 99.9%) as a white solid. LC/MS (ESI) m/z: 417 (M+H)⁺.

### Step 5:(2S,4R)—N—((R)-1-(4-carbamimidoylthiophen-2-yl)ethyl)-1-((4-(4-fluorophenoxy)benzoyl)glycyl)-4-methoxypyrrolidine-2-carboxamide (Compound 70)

To a mixture of (2S,4R)-1-((4-(4-fluorophenoxy)benzoyl)glycyl)-4-methoxypyrrolidine-2-carboxylic acid (60 mg, 0.14 mmol) and (R)-5-(1-aminoethyl)thiophene-3-carboximidamide (24 mg, 0.14 mmol) in DMF (5 mL) was added DIPEA (112 mg, 0.86 mmol) and T₃P (138 mg, 0.22 mmol, 50% wt. in EtOAc) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=5:1) and further purified by prep-HPLC to give (2S,4R)—N—((R)-1-(4-carbamimidoylthiophen-2-yl)ethyl)-1-((4-(4-fluorophenoxy)benzoyl)glycyl)-4-methoxypyrrolidine-2-carboxamide (10 mg, yield 12.23%) as a white solid. ¹H NMR (400 MHz, CD₃OD) δ 8.54 (s, 1H), 8.23 (dd, J=9.3, 1.6 Hz, 1H), 7.88-7.83 (m, 2H), 7.54 (dd, J=4.8, 3.5 Hz, 1H), 7.18-7.13 (m, 2H), 7.10-7.06 (m, 2H), 7.01-6.97 (m, 2H), 5.37-5.22 (m, 1H), 4.59-4.46 (m, 1H), 4.30-4.08 (m, 3H), 3.87-3.75 (m, 2H), 3.37 (d, J=4.0 Hz, 3H), 2.59-2.38 (m, 1H), 2.24-2.02 (m, 1H), 1.61 (dd, J=30.8, 7.0 Hz, 3H). LC/MS (ESI) m/z: 568 (M+H)⁺.

### Scheme 71: Synthesis of (2S,4R)—N—((R)-1-(4-carbamimidoylthiophen-2-yl)ethyl)-1-((9,9-difluoro-9H-fluorene-3-carbonyl)glycyl)-4-methoxypyrrolidine-2-carboxamide (Compound 71)

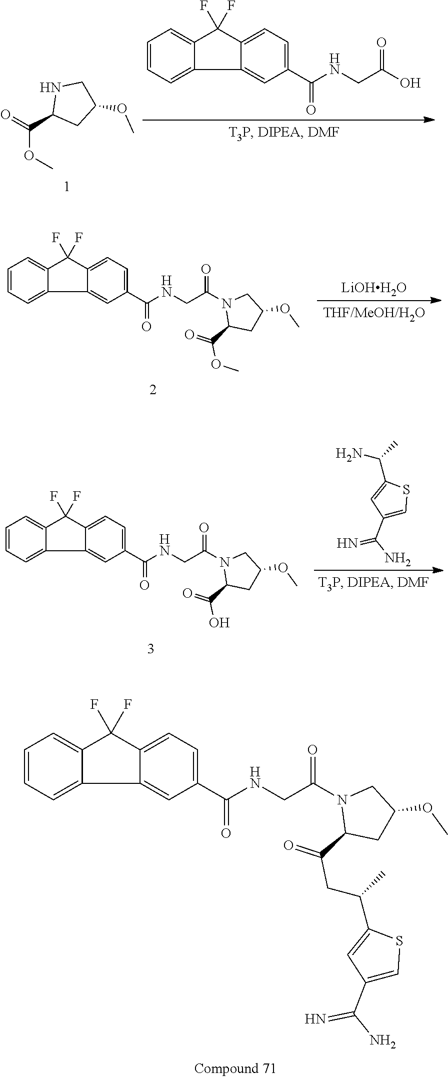

Compound 71 was prepared as a white solid from methyl (2S,4R)-4-methoxypyrrolidine-2-carboxylate, (9,9-difluoro-9H-fluorene-3-carbonyl)glycine, and (R)-5-(1-aminoethyl)thiophene-3-carboximidamide based on the procedures set forth in Scheme 3. ¹H NMR (400 MHz, CD₃OD) δ 8.49 (s, 1H), 8.27-8.14 (m, 2H), 7.86 (td, J=7.9, 1.4 Hz, 1H), 7.80-7.69 (m, 2H), 7.65 (d, J=7.5 Hz, 1H), 7.56 (dd, J=16.4, 9.0 Hz, 2H), 7.45 (t, J=7.5 Hz, 1H), 5.52-5.22 (m, 1H), 4.55 (dt, J=38.3, 7.9 Hz, 1H), 4.37-4.07 (m, 3H), 4.00-3.79 (m, 2H), 3.40-3.34 (m, 3H), 2.59-2.38 (m, 1H), 2.27-2.02 (m, 1H), 1.62 (dd, J=34.3, 7.0 Hz, 3H). LC/MS (ESI) m/z: 582 (M+H)⁺.

### Scheme 72: Synthesis of (1S,3S,5R)—N—((R)-1-(4-carbamimidoylthiophen-2-yl)ethyl)-2-((9,9-difluoro-9H-fluorene-3-carbonyl)glycyl)-5-(methoxymethyl)-2-azabicyclo[3.1.0]hexane-3-carboxamide (Compound 72)

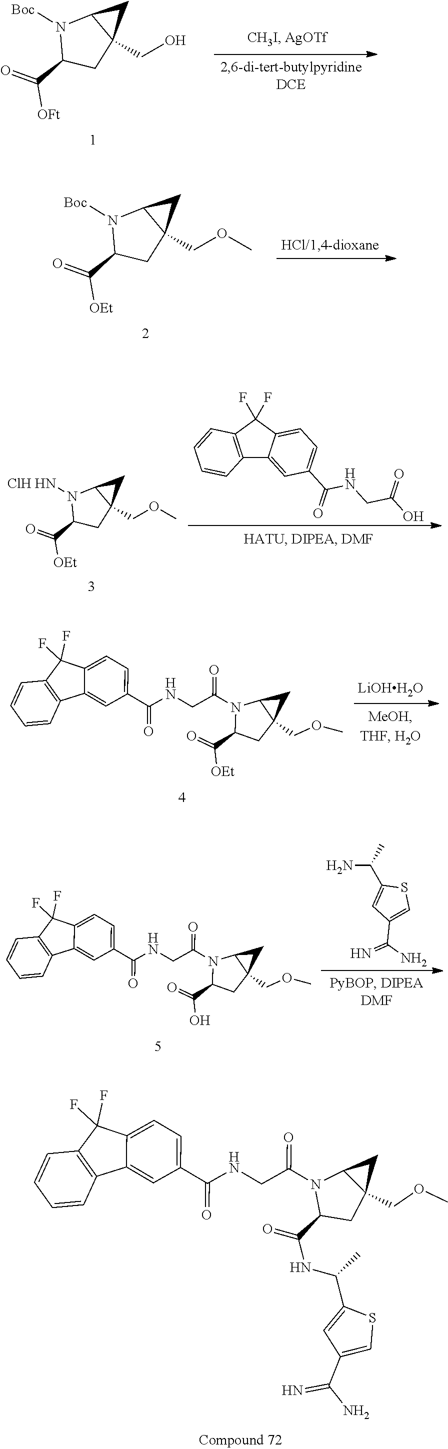

### Step 1: 2-(tert-butyl) 3-ethyl (1S,3S,5R)-5-(methoxymethyl)-2-azabicyclo[3.1.0]hexane-2,3-dicarboxylate (2)

To a solution of 2-(tert-butyl) 3-ethyl (1S,3S,5R)-5-(hydroxymethyl)-2-azabicyclo [3.1.0]hexane-2,3-dicarboxylate (200 mg, 0.70 mmol) in DCE (8 mL) was added AgOTf (450 mg, 2.5 mmol) followed by methyl iodide (500 mg, 5.0 mmol) and 2,6-di-tert-butylpyridine (0.40 mL, 2.5 mmol) and the mixture was stirred under N₂ atmosphere at 30° C. for 16 hours. The mixture was filtered, and the filtrate was concentrated to dryness. The residue was purified by flash chromatography on silica gel (PE:EtOAc=3:1) to give 2-(tert-butyl) 3-ethyl (1S,3S,5R)-5-(methoxymethyl)-2-azabicyclo[3.1.0]hexane-2,3-dicarboxylate (170 mg, yield 81.0%) as a colorless oil. LC/MS (ESI) (m/z): 300 (M+H)⁺.

### Step 2: ethyl (1S,3S,5R)-5-(methoxymethyl)-2-azabicyclo[3.1.0]hexane-3-carboxylate hydrochloride (3)

A mixture of 2-(tert-butyl) 3-ethyl (1 S,3S,5R)-5-(methoxymethyl)-2-azabicyclo[3.1.0]hexane-2,3-dicarboxylate (170 mg, 0.57 mmol) in HCl/1,4-dioxane (4 mL) was stirred at room temperature for 2 hours. The reaction mixture was concentrated to dryness under reduced pressure, co-evaporated with DCM and dried under vacuum to give ethyl (1S,3S,5R)-5-(methoxymethyl)-2-azabicyclo[3.1.0]hexane-3-carboxylate hydrochloride (130 mg, yield 96.7%) as a colorless oil, which was used directly in the next reaction without further purification. LC/MS (ESI) m/z: 200 (M+H)⁺.

### Step 3: ethyl (1S,3S,5R)-2-((9,9-difluoro-9H-fluorene-3-carbonyl)glycyl)-5-(methoxymethyl)-2-azabicyclo[3.1.0]hexane-3-carboxylate (4)

To a mixture of ethyl (1S,3S,5R)-5-(methoxymethyl)-2-azabicyclo[3.1.0]hexane-3-carboxylate hydrochloride (120 mg, 0.51 mmol) and (9,9-difluoro-9H-fluorene-3-carbonyl)glycine (122 mg, 0.40 mmol) in DMF (3 mL) was added DIPEA (324 mg, 2.50 mmol) and HATU (572 mg, 1.5 mmol) at 0° C. and the mixture was stirred at 25° C. for 3 hours. The mixture was diluted with water and extracted with EtOAc twice. The combined organic layers were washed with brine, dried over anhydrous Na₂SO₄, filtered, and concentrated under reduced pressure to dryness to give ethyl (1S,3S,5R)-2-((9,9-difluoro-9H-fluorene-3-carbonyl)glycyl)-5-(methoxymethyl)-2-azabicyclo[3.1.0]hexane-3-carboxylate (180 mg, yield 74.0%) as a colorless oil. LC/MS (ESI) m/z: 485 (M+H)⁺.

### Step 4: (1S,3S,5R)-2-((9,9-difluoro-9H-fluorene-3-carbonyl)glycyl)-5-(methoxymethyl)-2-azabicyclo[3.1.0]hexane-3-carboxylic acid (5)

To a solution of ethyl (1 S,3S,5R)-2-((9,9-difluoro-9H-fluorene-3-carbonyl) glycyl)-5-(methoxymethyl)-2-azabicyclo[3.1.0]hexane-3-carboxylate (180 mg, 0.37 mmol) in MeOH (2 mL) and THF (1 mL) was added a solution of LiOH·H₂O (47 mg, 1.12 mmol) in water (1 mL) and the mixture was stirred at room temperature for 2 hours. The mixture was diluted with water and washed with EtOAc twice. The aqueous layer was acidified with 0.5 M aq·HCl to pH-3 and extracted with EtOAc twice. The combined organic layers were washed with brine, dried over anhydrous Na₂SO₄, filtered, and concentrated to dryness to give (1S,3S,5R)-2-((9,9-difluoro-9H-fluorene-3-carbonyl)glycyl)-5-(methoxymethyl)-2-azabicyclo[3.1.0]hexane-3-carboxylic acid (139 mg, yield 82.0%) as a yellow solid. LC/MS (ESI) (m/z): 457 (M+H)⁺.

### Step 5: (1S,3S,5R)—N—((R)-1-(4-carbamimidoylthiophen-2-yl)ethyl)-2-((9,9-difluoro-9H-fluorene-3-carbonyl)glycyl)-5-(methoxymethyl)-2-azabicyclo[3.1.0]hexane-3-carboxamide (Compound 72)

To a mixture of (1 S,3S,5R)-2-((9,9-difluoro-9H-fluorene-3-carbonyl)glycyl)-5-(methoxymethyl)-2-azabicyclo[3.1.0]hexane-3-carboxylic acid (50 mg, 0.11 mmol) and (R)-5-(1-aminoethyl)thiophene-3-carboximidamide (28 mg, 0.16 mmol) in DMF (2 mL) was added DIPEA (71 mg, 0.55 mmol) and T₃P (83 mg, 0.13 mmol, 50% in EtOAc) at 0° C. and the mixture was stirred at room temperature for 2 hours. The mixture was diluted with water and extracted with EtOAc twice. The combined organic layers were washed with brine, dried over anhydrous Na₂SO₄, filtered, and concentrated under reduced pressure to dryness. The residue was purified by prep-TLC (DCM:MeOH=10:1) and further purified by prep-HPLC to give Compound 72 (4.1 mg, yield 6.2%) as a white solid. ¹HNMR (400 MHz, CD₃OD) δ 8.52 (s, 1H), 8.25-8.14 (m, 2H), 7.87 (dd, J=7.8, 1.5 Hz, 1H), 7.74 (dd, J=14.0, 7.6 Hz, 2H), 7.66 (d, J=7.2 Hz, 1H), 7.58 (t, J=7.6 Hz, 1H), 7.48 (dt, J=15.1, 4.4 Hz, 2H), 5.23 (q, J=6.7 Hz, 1H), 4.49 (d, J=16.6 Hz, 1H), 4.32 (d, J=16.5 Hz, 1H), 3.60 (dd, J=6.1, 2.7 Hz, 1H), 3.52 (d, J=10.3 Hz, 1H), 3.41 (d, J=10.3 Hz, 1H), 3.38 (s, 3H), 3.35 (d, J=4.2 Hz, 1H), 2.69 (t, J=12.1 Hz, 1H), 2.11 (dd, J=13.4, 3.7 Hz, 1H), 1.60 (t, J=13.2 Hz, 3H), 1.35 (dd, J=5.7, 2.6 Hz, 1H), 1.02 (t, J=5.5 Hz, 1H). LC/MS (ESI) m/z: 608 (M+H)⁺.

### Scheme 73. (S)—N-((4-carbamimidoylthiophen-2-yl)methyl)-7-((4′-fluoro-[1,1′-biphenyl]-4-carbonyl)glycyl)-1,4-dioxa-7-azaspiro[4.4]nonane-8-carboxamide (Compound 73)

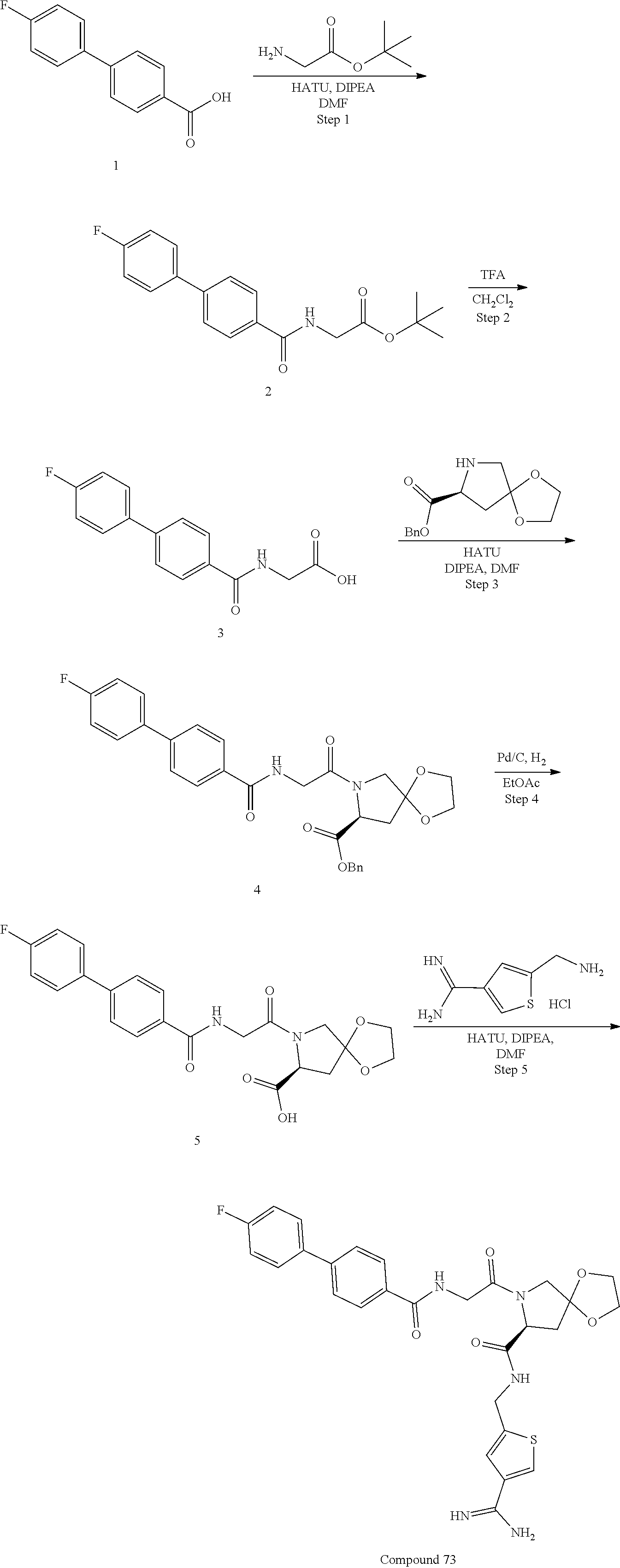

Compound 73 was prepared from 4-(4-fluorophenyl)benzoic acid, tert-butyl 2-aminoacetate, benzyl (1 S,3S,5S)-5-methyl-2-azabicyclo[3.1.0]hexane-3-carboxylate, and 5-(aminomethyl)thiophene-3-carboximidamide. HCl based on the procedures set forth in Scheme 1. LC/MS (ESI) m/z: 566 (M+H)⁺. ¹H NMR (400 MHz, DMSO-d₆) δ 9.02 (s, 1H), 8.74 (t, J=5.5 Hz, 2H), 8.62 (t, J=5.9 Hz, 1H), 8.42 (s, 1H), 8.36 (s, 1H), 7.95 (m, 3H), 7.78 (td, J=8.3, 5.8 Hz, 1H), 7.48 (s, 1H), 7.32 (t, J=8.8 Hz, 2H), 4.60-4.28 (m, 3H), 4.28-4.06 (m, 2H), 4.00-3.57 (m, 4H), 3.35 (d, J=11.3 Hz, 1H), 2.50 (t, J=2.0 Hz, 2H), 2.37-2.24 (m, 2H), 2.20-1.86 (m, 1H), 1.34-1.10 (m, 1H).

### Scheme 74. (S)—N-((4-carbamimidoylthiophen-2-yl)methyl)-7-((2-fluoro-4-phenoxybenzoyl)glycyl)-1,4-dioxa-7-azaspiro[4.4]nonane-8-carboxamide (Compound 74)

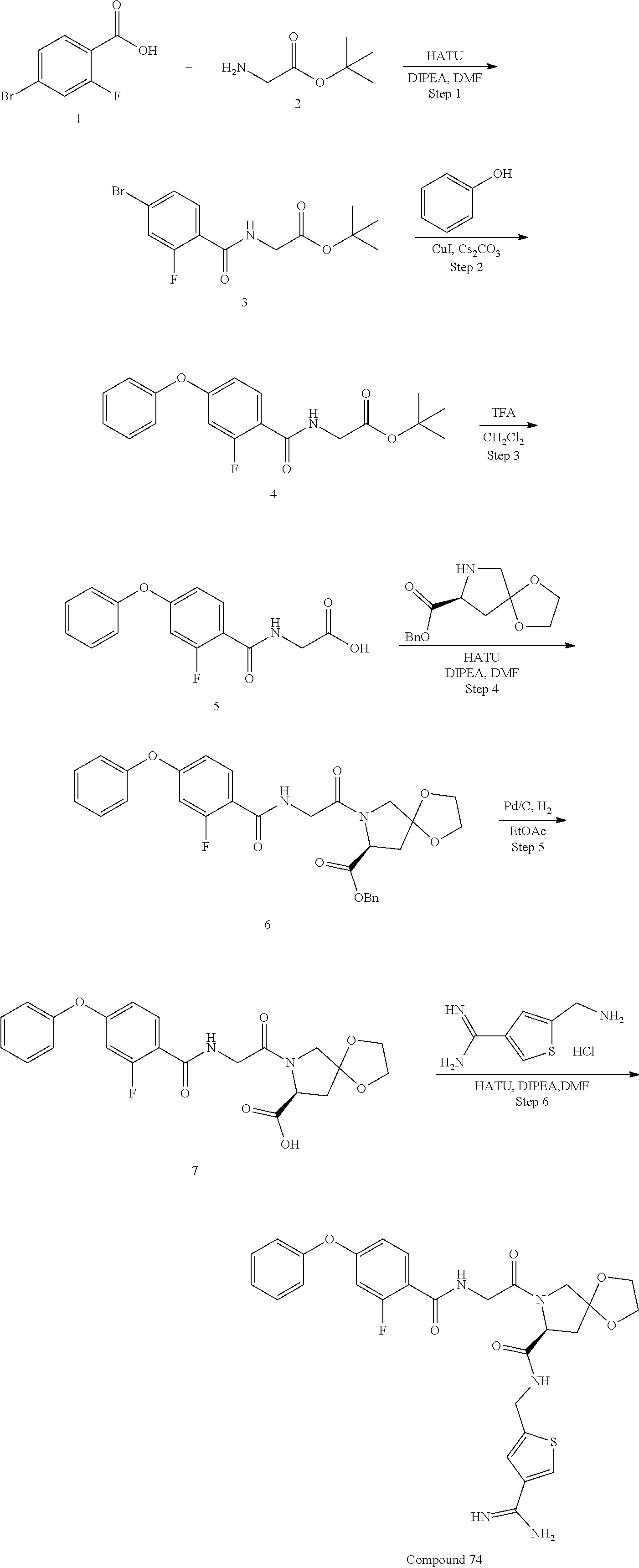

### Step 1: tert-butyl (4-bromo-2-fluorobenzoyl)glycinate (4)

To a solution of 4-bromo-2-fluoro-benzoic acid, 1 (0.5 g, 2.28 mmol, 1 equiv.) in DMF (2 mL) was added tert-butyl 2-aminoacetate (0.458 g, 2.73 mmol, 1.2 equiv.), HATU (1.13 g, 2.96 mmol, 1.3 equiv.) and DIPEA (1.19 mL, 6.84 mmol, 3 equiv.). The reaction was stirred at room temperature for 1 hour. Water (5 mL) was added and the solid collected by filtration and then dried to give 3 (0.6 g, 1.81 mmol, yield 79.26%) as a white solid. LC/MS (ESI) m/z: 333 (M+H)⁺.

### Step 2: tert-butyl (2-fluoro-4-phenoxybenzoyl)glycinate (4)

A mixture of 3 (0.5 g, 1.51 mmol, 1.0 equiv.), phenol (0.21 g, 2.27 mmol, 1.5 equiv.), dimethylaminoaceticacid. HCl (63 mg, 0.45 mmol, 0.3 equiv.), Cs₂CO₃ (1.97 g, 6.04 mmol, 4.0 equiv.), and copper(I)iodide (29 mg, 0.15 mmol, 0.1 equiv.) in dioxane (15 mL) were heated in a sealed tube overnight. The reaction was cooled to room temperature then diluted with water (25 mL) and EtOAc (25 mL). The two layers were separated, and the aqueous layer was extracted with EtOAc (10 mL×2). The combined organic extracts were washed with brine, dried over Na₂SO₄, and concentrated to give a yellow oil. Purification by Combi-Flash; 24 g column, solvent A=Hexanes, solvent B=EtOAc, 100% A to 5% B gave 4 (0.5 g, 1.45 mmol, 95.70%) as a white solid. LC/MS (ESI) m/z: 346 (M+H)+.

### Step 3: (2-fluoro-4-phenoxybenzoyl)glycine (5)

TFA (1.0 mL) was added to a solution of compound 4 (0.6 g, 1.74 mmol, 1.0 equiv.) in CH₂Cl₂ (3 mL) at ice-bath temperature. The reaction was stirred at room temperature for 3 hours and then concentrated to dryness to give compound 5 (0.5 g, 1.71 mmol, yield 98.50%) as a yellow solid which was used in the next step without further purification. LC/MS (ESI) m/z: 290 (M+H)+.

### Step 4: benzyl (S)-7-((2-fluoro-4-phenoxybenzoyl)glycyl)-1,4-dioxa-7-azaspiro[4.4]nonane-8-carboxylate (6)

To a solution of compound 5 (0.15 g, 0.52 mmol, 1.0 equiv.) in DMF (3 mL) was added benzyl (1S,3S,5S)-5-methyl-2-azabicyclo[3.1.0]hexane-3-carboxylate (0.15 g, 0.57 mmol, 1.1 equiv.), HATU (0.26 g, 0.68 mmol, 1.3 equiv.) and DIPEA (0.28 mL, 1.56 mmol, 3.0 equiv.). The reaction was stirred at room temperature for 30 minutes. Water (5 mL) was added, and the liquid decanted. The residue was taken up in EtOAc washed with brine, dried over Na₂SO₄, and concentrated. Purification by Combi Flash; 4 g column, solvent A=Hexanes, solvent B=EtOAc, 100% A to 25% B gave 6 (0.15 g, 0.28 mmol, yield 53.85%) as a brown oil. LC/MS (ESI) m/z: 535 (M+H)+.

### Step 5: (S)-7-((2-fluoro-4-phenoxybenzoyl)glycyl)-1,4-dioxa-7-azaspiro[4.4]nonane-8-carboxylic acid (7)

To a solution of 6 (0.15 g, 0.28 mmol, 1.0 equiv.) in MeOH (2 mL) was added 5% Pd/C (0.06 g, 0.028 mmol, 0.1 equiv.). The flask was evacuated and then backfilled with H₂ in a balloon. The reaction was stirred at room temperature overnight and then filtered through Celite pad. The filtrate was concentrated and dried in vacuo to give 7 (0.10 g, 0.23 mmol, yield 82.14%) as a brown solid which was used without further purification. LC/MS (ESI) m/z: 445 (M+H)⁺.

### Step 6: (S)—N-((4-carbamimidoylthiophen-2-yl)methyl)-7-((2-fluoro-4-phenoxybenzoyl)glyc yl)-1,4-dioxa-7-azaspiro[4.4]nonane-8-carboxamide (Compound 74)

To a solution of 7 (0.09 g, 0.20 mmol, 1.0 equiv.) in DMF (2 mL) was added 5-(aminomethyl)thiophene-3-carboximidamide. HCl (0.04 g, 0.21 mmol, 1.1 equiv.), HATU (0.09 g, 0.24 mmol, 1.2 equiv.), and DIPEA (0.1 mL, 0.57 mmol, 3.0 equiv.). The reaction mixture was stirred at room temperature for 30 minutes and then purified directly by HPLC to give Compound 74 (15 mg, 0.03 mmol, 13% yield). LC/MS (ESI) m/z: 582 (M+H)⁺. ¹H NMR (400 MHz, DMSO-d₆) δ 8.96 (s, 1H), 8.61 (t, J=5.9 Hz, 1H), 8.34 (d, J=8.8 Hz, 1H), 8.14 (d, J=6.3 Hz, 2H), 7.76 (t, J=8.7 Hz, 2H), 7.53-7.43 (m, 3H), 7.26 (t, J=7.4 Hz, 2H), 7.14 (d, J=8.0 Hz, 2H), 6.99-6.79 (m, 3H), 4.50 (d, J=5.7 Hz, 1H), 4.41 (dd, J=17.5, 6.8 Hz, 3H), 4.16 (dd, J=17.0, 4.9 Hz, 1H), 4.03 (dd, J=17.0, 5.1 Hz, 1H), 3.85-3.67 (m, 2H), 2.97-2.84 (m, 1H), 2.31 (dd, J=13.3, 8.8 Hz, 1H), 2.05 (dd, J=13.1, 6.4 Hz, 1H).

### Scheme 75: (S)-7-((4-(4-fluorophenoxy)benzoyl)glycyl)-N-((4-(5-(trifluoromethyl)-1H-pyrazol-3-yl)thiophen-2-yl)methyl)-1,4-dioxa-7-azaspiro[4.4]nonane-8-carboxamide (Compound 76)

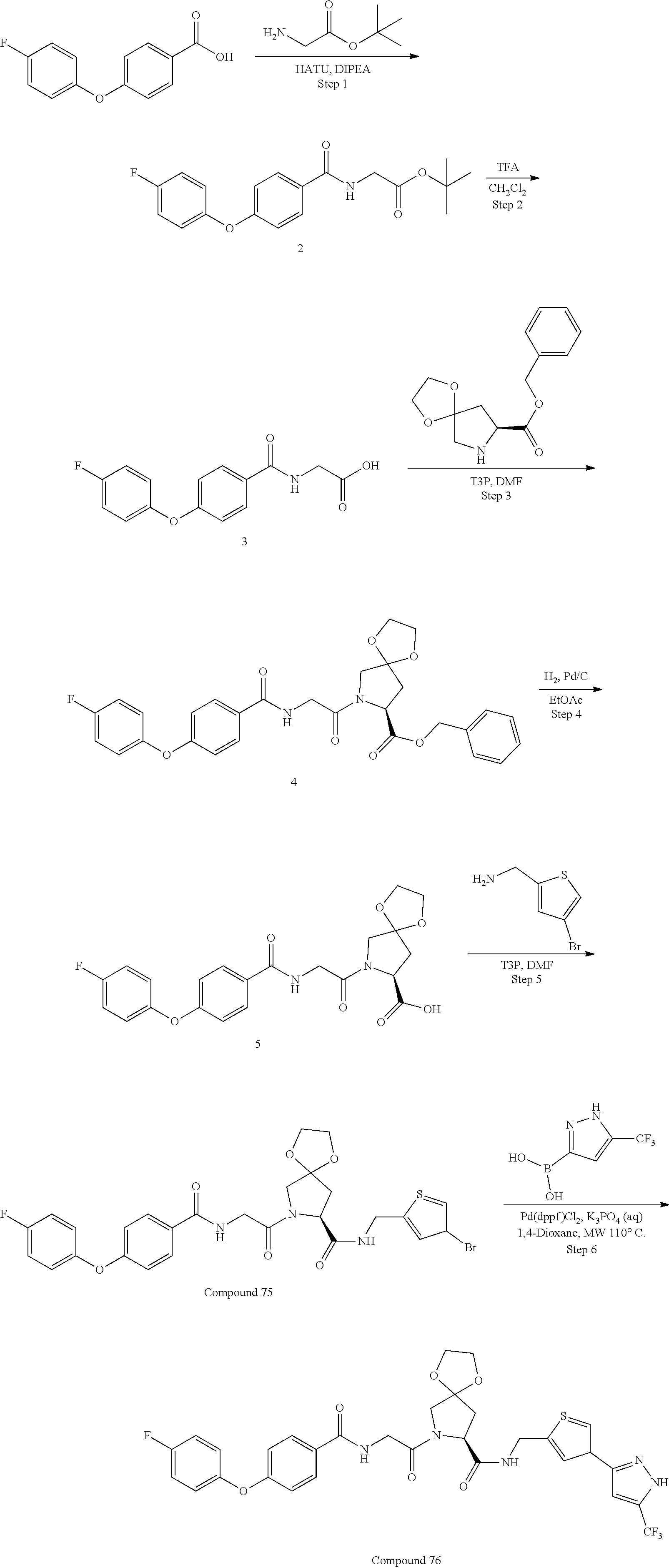

### Step 1: tert-butyl (4-(4-fluorophenoxy)benzoyl)glycinate (2)

To a mixture of 4-(4-fluorophenoxy)benzoic acid, 1 (1.00 g, 4.31 mmol, 1.0 equiv.) and tert-butyl 2-aminoacetate (1.08 g, 6.44 mmol, 1.5 equiv.) in DMF (5.00 mL) was added DIPEA (2.25 mL, 12.9 mmol, 3.0 equiv.) and HATU (3.28 g, 8.63 mmol, 2.0 equiv.). The mixture was stirred at 50° C. under Argon atmosphere for 2 h. The mixture was diluted with H₂O and extracted with EtOAc (3×25 mL). The combined organic extracts were dried over anhydrous Na₂SO₄, filtered, and concentrated to dryness under reduced pressure. Purification by Combi Flash; 24 g gold column, 0-10% MeOH in DCM, to obtain tert-butyl 2-[[4-(4-fluorophenoxy)benzoyl]amino]acetate, 2 (1.27 g, 3.68 mmol, 85.4% yield) as a beige solid. LC/MS (ESI) m/z: 346 (M+H)⁺.

### Step 2: (4-(4-fluorophenoxy)benzoyl)glycine (3)

To a solution of tert-butyl 2-[[4-(4-fluorophenoxy)benzoyl]amino]acetate, 2 (1.27 g, 3.68 mmol, 1.0 equiv.) in DCM (5.00 mL) was added Trifluoroacetic acid (2.50 mL, 33.1 mmol, 9.0 equiv.). The reaction mixture was stirred at ambient temperature for 5 h. LC-MS indicated the completion of the reaction. The solvent was removed under reduced pressure and the residue was further dried under high vacuum to obtain the desired product 2-[[4-(4-fluorophenoxy)benzoyl]amino]acetic acid, 3 (1.06 g, 3.66 mmol, 99.6% yield) as a beige solid. The product was used to the next step without further purification. LC/MS (ESI) m/z: 290 (M+H)⁺.

### Step 3: benzyl (S)-7-((4-(4-fluorophenoxy)benzoyl)glycyl)-1,4-dioxa-7-azaspiro[4.4]nonane-8-carboxylate (4)

To a solution of 2-[[4-(4-fluorophenoxy)benzoyl]amino]acetic acid, 3 (1.06 g, 3.67 mmol, 1.10 equiv.) and benzyl 1,4-dioxa-7-azaspiro[4.4]nonane-8-carboxylate (0.900 g, 3.42 mmol, 1.0 equiv.) in DMF (6 mL) was added DIPEA (1.80 mL, 10.3 mmol, 3.0 equiv.) and 1-Propanephosphonic anhydride solution (50% in Ethyl acetate, Approx 1.7 Mol/L (3.00 mL, 4.99 mmol, 1.0 equiv.) at room temperature under Argon atmosphere. The solution was stirred at room temperature for 2 h. LC-MS indicated the completion of the reaction. The reaction mixture was slowly added to water while stirring. Solid formation was observed. The dried solids were further purified by ISCO column chromatographic purification system (24 g gold column) using 0-70% EtOAc in Hexane as eluent to obtain the desired product benzyl 7-[2-[[4-(4-fluorophenoxy)benzoyl]amino]acetyl]-1,4-dioxa-7-azaspiro[4.4]nonane-8-carboxylate, 4 (0.514 g, 0.962 mmol, 28.1% yield) as a beige solid. LC/MS (ESI) m/z: 535 (M+H)⁺.

### Step 4: (S)-7-((4-(4-fluorophenoxy)benzoyl)glycyl)-1,4-dioxa-7-azaspiro[4.4]nonane-6-carboxylic acid (5)

To a solution of benzyl 7-[2-[[4-(4-fluorophenoxy)benzoyl]amino]acetyl]-1,4-dioxa-7-azaspiro[4.4]nonane-8-carboxylate, 6 (0.500 g, 0.935 mmol, 1.0 equiv.) in Ethyl acetate (7.00 mL) was added Palladium on carbon (100 mg, 0.094 mmol, 0.10 equiv.). The flask was evacuated and backfilled with HYDROGEN (99.99 mass %) in a balloon and stirred under positive pressure of HYDROGEN (99.99 mass %) gas for 4 h. The mixture was filtered, and the filtrate was concentrated to dryness under reduced pressure to give 7-[2-[[4-(4-fluorophenoxy)benzoyl]amino]acetyl]-1,4-dioxa-7-azaspiro[4.4]nonane-8-carboxylic acid, 5 (0.390 g, 0.878 mmol, 93.8% yield), which was used in the next step without further purification. LC/MS (ESI) m/z: 445 (M+H)⁺.

### Step 5: (S)—N-((4-bromothiophen-2-yl)methyl)-7-((4-(4-fluorophenoxy)benzoyl)glycyl)-1,4-dioxa-7-azaspiro[4.4]nonane-8-carboxamide (Compound 75)

To a solution of (4-bromo-2-thienyl)methanamine (0.065 g, 0.338 mmol, 1.5 equiv.) and 7-[2-[[4-(4-fluorophenoxy)benzoyl]amino]acetyl]-1,4-dioxa-7-azaspiro[4.4]nonane-8-carboxylic acid, 5 (0.100 g, 0.225 mmol, 1.0 equiv.) in DMF (5.00 mL) was added DIPEA (0.10 mL, 0.57 mmol, 2.5 equiv.) and 1-Propanephosphonic anhydride solution (50% in Ethyl acetate, Approx 1.7 Mol/L (0.30 mL, 0.50 mmol, 2.2 equiv.) under argon atmosphere at room temperature and it was stirred for 2 h. LC-MS indicated the completion of the reaction. The solution was then added to half-saturated NH₄Cl (aq) solution while stirring. Solid formation was observed. The resulting solid was filtered, dried and further purified by ISCO column chromatographic purification system (12 g gold column) using 0-5% MeOH in DCM to obtain desired product N-[(4-bromo-2-thienyl)methyl]-7-[2-[[4-(4-fluorophenoxy)benzoyl]amino]acetyl]-1,4-dioxa-7-azaspiro[4.4]nonane-8-carboxamide, Compound 75 (0.130 g, 0.210 mmol, 93.4% yield) as a beige solid. LC/MS (ESI) m/z: 618 (M+H)⁺. ¹H NMR (400 MHz, DMSO) δ 2.01-2.10 (dd, J=13.1, 6.3 Hz, 1H), 2.28-2.38 (dd, J=13.2, 8.9 Hz, 1H), 3.61-3.72 (dd, J=15.3, 11.2 Hz, 1H), 3.77-3.84 (d, J=10.8 Hz, 1H), 3.88-4.03 (m, 5H), 4.06-4.21 (m, 1H), 4.33-4.52 (m, 3H), 6.94-7.07 (m, 3H), 7.13-7.22 (dd, J=9.1, 4.4 Hz, 2H), 7.24-7.34 (t, J=8.7 Hz, 2H), 7.45-7.55 (m, 1H), 7.87-7.94 (d, J=8.4 Hz, 2H), 8.48-8.55 (t, J=6.0 Hz, 1H), 8.56-8.64 (t, J=5.6 Hz, 1H).

### Step 6: (S)-7-((4-(4-fluorophenoxy)benzoyl)glycyl)-N-((4-(5-(trifluoromethyl)-1H-pyrazol-3-yl)thiophen-2-yl)methyl)-1,4-dioxa-7-azaspiro[4.4]nonane-8-carboxamide (Compound 76)

To a solution of N-[(4-bromo-2-thienyl)methyl]-7-[2-[[4-(4-fluorophenoxy)benzoyl]amino]acetyl]-1,4-dioxa-7-azaspiro[4.4]nonane-8-carboxamide, 6 (0.065 g, 0.105 mmol, 1.0 equiv.) and [5-(trifluoromethyl)-1H-pyrazol-3-yl]boronic acid (0.030 g, 0.167 mmol, 1.6 equiv.) in 1,4-Dioxane (5.00 mL, 58.6 mmol, 100 mass %) was added 2 M K₃PO₄ in H₂O (0.15 mL, 0.30 mmol, 2.00 mol/L, 2.9 equiv.). Ar gas was passed through the solution for 2 min followed by the addition Pd(dppt)Cl₂ (0.010 g, 0.0130 mmol, 0.12 equiv.). The mixture was de-gassed one more time by purging with Ar. The tube was sealed and placed in a microwave synthesizer for 30 mins at 110° C. LC/MS indicated the completion of the reaction. The mixture was then passed through a celite plug and the filtrate was concentrated. The residue was purified by ISCO column chromatographic purification system (12 g gold column) using 0-5% MeOH in DCM followed by trituration of the desired fractions in MeCN to obtain desired product Compound 76 (0.010 g, 0.0148 mmol, 14.1% yield) as a beige solid. LC/MS (ESI) m/z: 674 (M+H)⁺. ¹H NMR (400 MHz, DMSO) δ 2.00-2.10 (dd, J=13.1, 6.8 Hz, 1H), 2.27-2.37 (dd, J=13.2, 8.8 Hz, 1H), 3.59-3.83 (m, 2H), 3.85-4.01 (m, 5H), 4.05-4.20 (m, 1H), 4.35-4.74 (m, 3H), 6.93-6.97 (s, 1H), 6.97-7.04 (dd, J=8.4, 6.0 Hz, 2H), 7.10-7.19 (dd, J=9.0, 4.4 Hz, 2H), 7.23-7.31 (t, J=8.6 Hz, 2H), 7.36-7.44 (m, 1H), 7.78-7.84 (d, J=12.1 Hz, 1H), 7.84-7.91 (d, J=8.5 Hz, 2H), 8.49-8.62 (m, 2H), 13.89-13.94 (s, 1H).

### Scheme 76: (S)-7-(([1,1′-biphenyl]-4-carbonyl)glycyl)-N-((4-carbamimidoylthiophen-2-yl)methyl)-1,4-dioxa-7-azaspiro[4.4]nonane-8-carboxamide (Compound 77)

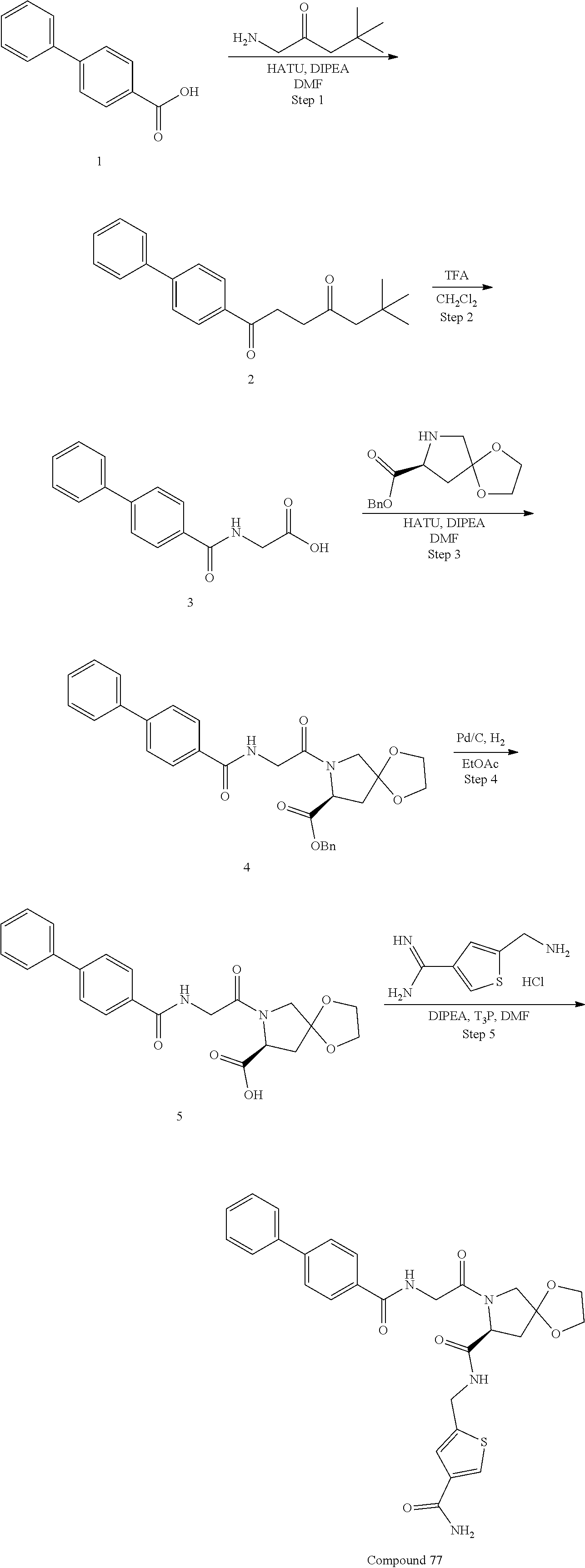

Compound 77 was prepared as a white solid from 4-phenylbenzoic acid, tert-butyl 2-aminoacetate, benzyl (1 S,3S,5S)-5-methyl-2-azabicyclo[3.1.0]hexane-3-carboxylate, and 5-(aminomethyl)thiophene-3-carboximidamide. HCl based on the procedures set forth in Scheme 1. LC/MS (ESI) m/z: 548 (M+H)⁺. LC/MS (ESI) m/z: 546 (M+H)⁺. ¹H NMR (400 MHz, DMSO-d₆) δ 9.04 (s, 1H), 8.87-8.21 (m, 1H), 7.96 (t, J=7.1 Hz, 1H), 7.76 (dd, J=16.9, 7.7 Hz, 1H), 7.60-7.22 (m, 1H), 4.73-4.70 (m, 1H), 4.52-4.32 (m, 2H), 4.44-4.40 (m, 2H), 4.40-3.98 (m, 1H), 4.19 (d, J=17.9 Hz, 1H), 4.03 (s, 1H), 4.00-3.87-3.91 (m, 4H), 3.80 (s, 1H), 3.71 (m, 1H), 3.64-3.60 (m, 1H), 2.51 (d, J=4.3 Hz, 1H), 2.34-2.29 (m, 1H), 2.20-2.10 (m, 1H), 2.05 (dd, J=13.9, 7.5 Hz, 1H).

### Scheme 77: Synthesis of (S)-7-((anthracene-2-carbonyl)glycyl)-N—((R)-1-(4-carbamimidoylthiophen-2-yl)ethyl)-1,4-dioxa-7-azaspiro[4.4]nonane-8-carboxamide (Compound 78)

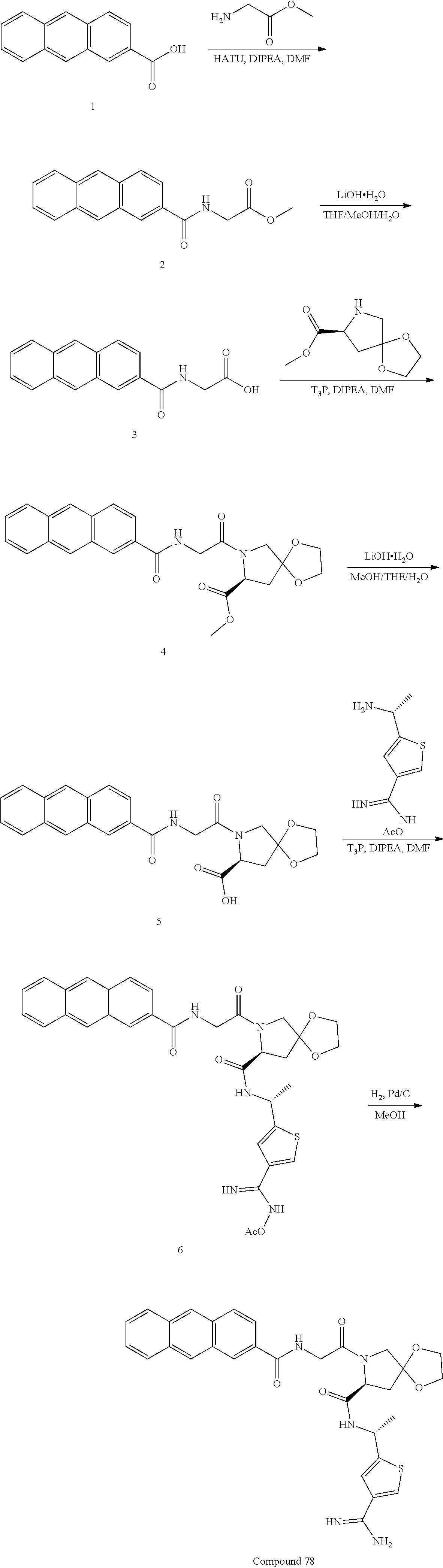

### Step 1: methyl (anthracene-2-carbonyl)glycinate (2)

To a mixture of anthracene-2-carboxylic acid (500 mg, 2.25 mmol) and methyl glycinate (301 mg, 3.37 mmol) in DMF (10 mL) was added HATU (1.3 g, 3.37 mmol) and DIPEA (872 mg, 6.75 mmol) under N₂ atmosphere and the reaction mixture was stirred at room temperature for 2 hours. The mixture was diluted with EtOAc and washed with water and brine, dried over anhydrous Na₂SO₄, filtered, and concentrated under reduced pressure to dryness. The residue was purified by flash chromatography on silica gel (PE:EtOAc=5:1) to give methyl (anthracene-2-carbonyl)glycinate (600 mg, yield 90.9%) as a white solid. LC/MS (ESI) m/z:294 (M+H)⁺.

### Step 2: (anthracene-2-carbonyl)glycine (3)

To a solution of methyl (anthracene-2-carbonyl)glycinate (600 mg, 2.05 mmol) in THF/MeOH/H₂O (12 mL,4/1/1) was added LiOH (172 mg, 4.09 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography on silica gel (DCM:MeOH=20:1) to give (anthracene-2-carbonyl)glycine (560 mg, yield 98.0%) as a white solid. LC/MS (ESI) m/z:280 (M+H)⁺.

### Step 3: methyl (S)-7-((anthracene-2-carbonyl)glycyl)-1,4-dioxa-7-azaspiro[4.4]nonane-6-carboxylate (4)

To a mixture of (anthracene-2-carbonyl)glycine (360 mg, 1.29 mmol) and methyl (S)-1,4-dioxa-7-azaspiro[4.4]nonane-8-carboxylate (241 mg, 1.29 mmol) in DMF (10 mL) was added DIPEA (999 mg, 7.73 mmol) and T₃P (1.2 g, 1.93 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography on silica gel (PE:EtOAc=3:1) to give methyl (S)-7-((anthracene-2-carbonyl)glycyl)-1,4-dioxa-7-azaspiro[4.4]nonane-8-carboxylate (240 mg, yield 41.5%) as a white solid. LC/MS (ESI) m/z: 449 (M+H)⁺.

### Step 4: (S)-7-((anthracene-2-carbonyl)glycyl)-1,4-dioxa-7-azaspiro[4.4]nonane-8-carboxylic acid (5)

To a solution of methyl (S)-7-((anthracene-2-carbonyl)glycyl)-1,4-dioxa-7-azaspiro[4.4]nonane-8-carboxylate (240 mg, 0.54 mmol) in THF/MeOH/H₂O (3 mL,4/1/1) was added LIOH (45 mg, 1.07 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography on silica gel (DCM:MeOH=10:1) to give (S)-7-((anthracene-2-carbonyl)glycyl)-1,4-dioxa-7-azaspiro[4.4]nonane-8-carboxylic acid (200 mg, yield 86.0%) as a white solid. LC/MS (ESI) m/z:435 (M+H)⁺.

### Step 5: (S)—N—((R)-1-(4-(N-acetoxycarbamimidoyl)thiophen-2-yi)ethyl)-7-((anthracene-2-carbonyl)glycyl)-1,4-dioxa-7-azaspiro[4.4]nonane-8-carboxamide (6)

To a mixture of (S)-7-((anthracene-2-carbonyl)glycyl)-1,4-dioxa-7-azaspiro[4.4]nonane-8-carboxylic acid (50 mg, 0.12 mmol) and (R)—N-acetoxy-5-(1-aminoethyl)thiophene-3-carboximidamide (31 mg, 0.14 mmol) in DMF (3 mL) was added DIPEA (89 mg, 0.69 mmol) and T₃P (110 mg, 0.17 mmol, 50% wt. in EtOAc) under N₂ atmosphere and the mixture was stirred at room temperature for 3 hours. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography on silica gel (PE:EtOAc=1:1) to give (S)—N—((R)-1-(4-(N-acetoxycarbamimidoyl)thiophen-2-yl)ethyl)-7-((anthracene-2-carbonyl)glycyl)-1,4-dioxa-7-azaspiro[4.4]nonane-8-carboxamide (50 mg, yield 67.5%) as a white solid. LC/MS (ESI) m/z: 644 (M+H)⁺.

### Step 6: (S)-7-((anthracene-2-carbonyl)glycyl)-N—((R)-1-(4-carbamimidoylthiophen-2-yl) ethyl)-1,4-dioxa-7-azaspiro[4.4]nonane-8-carboxamide (Compound 78)

To a solution of (S)—N—((R)-1-(4-(N-acetoxycarbamimidoyl)thiophen-2-yl)ethyl)-7-((anthracene-2-carbonyl)glycyl)-1,4-dioxa-7-azaspiro[4.4]nonane-8-carboxamide (50 mg, 0.078 mmol) in MeOH (3 mL) was added Pd/C (100 mg, 10% wt.) and the reaction mixture was stirred under a H₂ balloon at 30° C. overnight. The mixture was filtered, and the filtrate was concentrated under reduced pressure to dryness. The residue was purified by prep-TLC (DCM:MeOH=5:1) and further purified by prep-HPLC to give Compound 78 (8 mg, yield 17.6%) as a white solid. ¹HNMR (400 MHz, CD₃OD) δ 8.61 (d, J=4.6 Hz, 2H), 8.54 (d, J=10.2 Hz, 1H), 8.13-8.06 (m, 4H), 7.92-7.84 (m, 1H), 7.57-7.50 (m, 3H), 5.32 (dt, J=20.7, 7.2 Hz, 1H), 4.60 (dd, J=8.8, 6.5 Hz, 1H), 4.24 (d, J=2.8 Hz, 1H), 4.06-3.94 (m, 5H), 3.87-3.78 (m, 2H), 2.45 (dd, J=13.3, 8.9 Hz, 1H), 2.23 (dd, J=13.1, 6.5 Hz, 1H), 1.61 (t, J=18.9 Hz, 3H). LC/MS (ESI) m/z:586 (M+H)⁺.

### Scheme 78: Synthesis of (2S,4R*)—N—((R)-1-(4-carbamimidoylthiophen-2-yl)ethyl)-1-((4-(4-fluorophenoxy)benzoyl)glycyl)-4-(methoxymethyl)pyrrolidine-2-carboxamide (Compound 79)

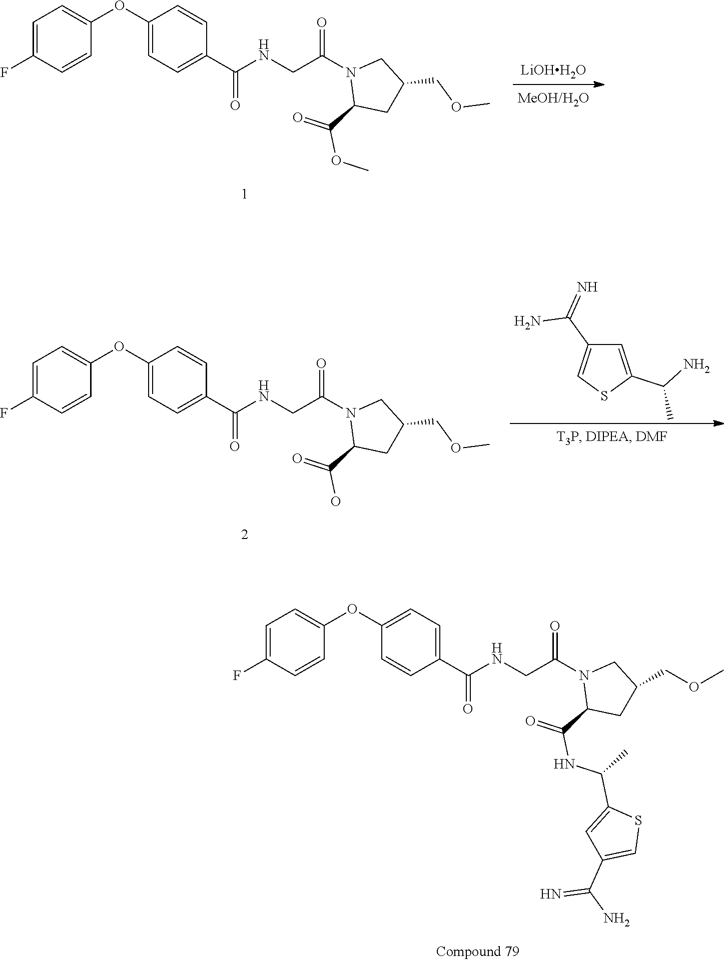

Compound 79 was prepared as a white solid from methyl (2S,4R*)-1-((4-(4-fluorophenoxy)benzoyl)glycyl)-4-(methoxymethyl)pyrrolidine-2-carboxylate and (R)-5-(1-aminoethyl)thiophene-3-carboximidamide based on the procedures set forth in Scheme 3. ¹H NMR (400 MHz, CD₃OD) δ 8.54 (s, 1H), 8.23 (dd, J=7.1, 1.6 Hz, 1H), 7.88-7.83 (m, 2H), 7.52-7.48 (m, 1H), 7.19-7.13 (m, 2H), 7.11-7.07 (m, 2H), 7.01-6.98 (m, 2H), 5.26 (q, J=6.9 Hz, 1H), 4.54 (dd, J=8.0, 4.0 Hz, 1H), 4.20-4.07 (m, 2H), 3.88 (dd, J=9.9, 7.7 Hz, 1H), 3.49-3.39 (m, 3H), 3.37-3.34 (m, 3H), 2.83-2.60 (m, 1H), 2.20-2.04 (m, 2H), 1.57 (d, J=7.0 Hz, 3H). LC/MS (ESI) m/z: 582 (M+H)⁺.

### Scheme 79: Synthesis of (2S,4S*)—N—((R)-1-(4-carbamimidoylthiophen-2-yl)ethyl)-1-((4-(4-fluorophenoxy)benzoyl)glycyl)-4-(methoxymethyl)pyrrolidine-2-carboxamide (Compound 80)

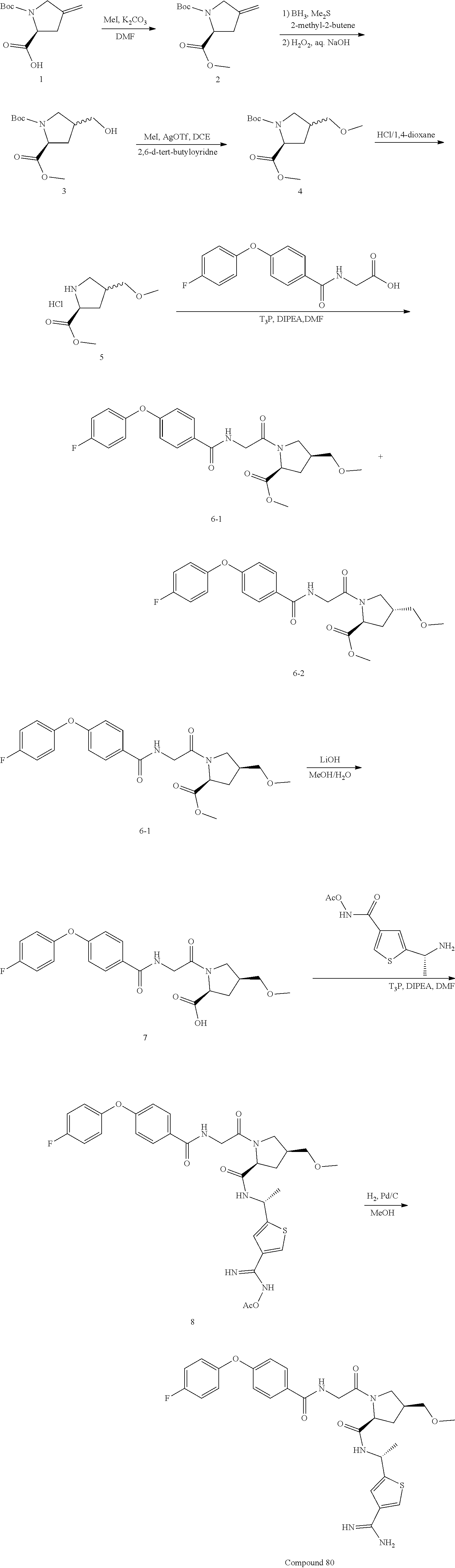

### Step 1: 1-(tert-butyl) 2-methyl (S)-4-methylenepyrrolidine-1,2-dicarboxylate (2)

To a solution of (S)-1-(tert-butoxycarbonyl)-4-methylenepyrrolidine-2-carboxylic acid (5 g, 22 mmol) in DMF (50 mL) was added Cs₂CO₃ (22 g, 66 mmol) and MeI (9.4 g, 66 mmol) under N₂ atmosphere and the mixture was stirred at room temperature overnight. The mixture was diluted with water and extracted with EtOAc twice. The combined organic layers were washed with brine, dried over anhydrous Na₂SO₄, filtered, and concentrated under reduced pressure to dryness. The residue was purified by column chromatography on silica gel (PE:EtOAc=2:1) to give 1-(tert-butyl) 2-methyl (S)-4-methylenepyrrolidine-1,2-dicarboxylate (4.1 g, yield 77.3%) as a colorless oil. LCMS (ESI) m/z: 242 (M+H)⁺.

### Step 2: 1-(tert-butyl) 2-methyl (2S)-4-(hydroxymethyl)pyrrolidine-1,2-dicarboxylate (3)

To a solution of 2-methylbut-2-ene (2.3 g, 33.2 mmol) in THF (10 mL) was added BH₃·Me₂S complex (1.7 mL, 17 mmol) drop-wisely at 0° C. and the mixture was stirred at 0° C. for 2 hours. The above mixture was added to a solution of 1-(tert-butyl) 2-methyl (S)-4-methylenepyrrolidine-1,2-dicarboxylate (2.0 g, 8.3 mmol) in THF (10 mL) drop-wisely at 0° C. and the reaction mixture was stirred at room temperature for 20 hours. Ethanol (0.5 mL) was added, and the mixture was cooled to −10° C. 3M aq.NaOH (2.8 mL) was added rapidly to the mixture followed by drop-wise addition of H₂O₂ (2.8 mL, 30% wt.) and the resulting mixture was stirred at room temperature for 3 hours. The organic layer was separated, and the aqueous layer was extracted with MTBE twice. The combined organic layers were washed with brine, dried over anhydrous Na₂SO₄, filtered, and concentrated under reduced pressure to dryness. The residue was purified by column chromatography on silica gel (PE:EtOAc=3:1) to give 1-(tert-butyl) 2-methyl (2S)-4-(hydroxymethyl)pyrrolidine-1,2-dicarboxylate (420 mg, yield 19.5%) as a colorless oil. LCMS (ESI) m/z: 261 (M+H)⁺.

### Step 3: 1-(tert-butyl) 2-methyl (2S)-4-(methoxymethyl)pyrrolidine-1,2-dicarboxylate (4)

To a mixture of 1-(tert-butyl) 2-methyl (2S)-4-(hydroxymethyl)pyrrolidine-1,2-dicarboxylate (420 mg, 1.6 mmol) and 2,6-di-tert-butylpyridine (459 mg, 2.4 mmol) in DCE (4 mL) was added AgOTf (617 mg, 2.4 mmol) and MeI (780 mg, 4.9 mmol) under N₂ atmosphere and the mixture was stirred at room temperature overnight. The mixture was diluted with water and extracted with EtOAc twice. The combined organic layers were washed with brine, dried over anhydrous Na₂SO₄, filtered, and concentrated under reduced pressure to dryness. The residue was purified by column chromatography on silica gel (PE:EtOAc=4:1) to give 1-(tert-butyl) 2-methyl (2S)-4-(methoxymethyl)pyrrolidine-1,2-dicarboxylate (300 mg, yield 68.6%) as a colorless oil. LCMS (ESI) m/z: 274 (M+H)⁺.

### Step 4: methyl (2S)-4-(methoxymethyl)pyrrolidine-2-carboxylate hydrochloride (5)

A solution of 1-(tert-butyl) 2-methyl (2S)-4-(methoxymethyl)pyrrolidine-1,2-dicarboxylate (300 mg, 1.1 mmol) in HCl/1,4-dioxane (3 mL, 4M) was stirred under N₂ atmosphere at room temperature for 30 minutes. The reaction mixture was concentrated under reduced pressure to dryness to give methyl (2S)-4-(methoxymethyl)pyrrolidine-2-carboxylate (270 mg, yield 100%) as a yellow solid, which was used directly in the next reaction without further purification. LC/MS (ESI) m/z: 174 (M+H)⁺.

### Step 5: methyl (2S)-1-((4-(4-fluorophenoxy)benzoyl)glycyl)-4-(methoxymethyl)pyrrolidine-2-carboxylate (6-1 and 6-2)

To a mixture of methyl (2S)-4-(methoxymethyl)pyrrolidine-2-carboxylate hydrochloride (157 mg, 0.75 mmol) and (4-(4-fluorophenoxy)benzoyl)glycine (217 mg, 0.75 mmol) in DMF (2.5 mL) was added DIPEA (580 mg, 4.5 mmol) and T₃P (620 mg, 0.98 mmol, 50% wt. in EtOAc) at 0° C. under N₂ atmosphere and the mixture was stirred at 25° C. for 2 hours. The mixture was diluted with EtOAc and washed with water twice. The combined organic layers were dried over anhydrous Na₂SO₄, filtered, and concentrated to dryness under reduced pressure. The residue was purified by column chromatography on silica gel (PE:EtOAc=2:1) and SFC to give methyl (2S,4S*)-1-((4-(4-fluorophenoxy)benzoyl)glycyl)-4-(methoxymethyl)pyrrolidine-2-carboxylate (60 mg, yield 18.7%) and methyl (2S,4R*)-1-((4-(4-fluorophenoxy)benzoyl)glycyl)-4-(methoxymethyl)pyrrolidine-2-carboxylate (100 mg, yield 30.7%) as a colorless oil. LC/MS (ESI) m/z: 445 (M+H)⁺.

### Step 6: (2S,4S*)-1-((4-(4-fluorophenoxy)benzoyl)glycyl)-4-(methoxymethyl)pyrrolidine-2-carboxylic acid (7)

A solution of methyl (2S,4S*)-1-((4-(4-fluorophenoxy)benzoyl)glycyl)-4-(methoxymethyl)pyrrolidine-2-carboxylate (60 mg, 0.14 mmol) in MeOH (2 mL) and water (1 mL) was added LiOH·H₂O (33 mg, 0.81 mmol) at 0° C. and the mixture was stirred at room temperature for 2 hours. The mixture was acidified with 1 N aq·HCl to pH˜3, extracted with EtOAc twice. The combined organic layers were washed with brine, dried over anhydrous Na₂SO₄, filtered, and concentrated under reduced pressure to dryness to give (2S,4S*)-1-((4-(4-fluorophenoxy)benzoyl)glycyl)-4-(methoxymethyl)pyrrolidine-2-carboxylic acid (45 mg, yield 77.5%) as a colorless oil, which was used directly in the next step. LC/MS (ESI) (m/z): 431 (M+H)⁺.

### Step 7: (2S,4S*)—N—((R)-1-(4-(N-acetoxycarbamimidoyl)thiophen-2-yl)ethyl)-1-((4-(4-fluorophenoxy)benzoyl)glycyl)-4-(methoxymethyl)pyrrolidine-2-carboxamide (8)

To a mixture of (2S,4S*)-1-((4-(4-fluorophenoxy)benzoyl)glycyl)-4-(methoxymethyl)pyrrolidine-2-carboxylic acid (40 mg, 0.093 mmol) and (R)—N-acetoxy-5-(1-aminoethyl)thiophene-3-carboximidamide (24 mg, 0.14 mmol) in DMF (1 mL) was added DIPEA (72 mg, 0.56 mmol) and T₃P (76 mg, 0.12 mmol, 50% in EtOAc) at 0° C. and the mixture was stirred under N₂ atmosphere at room temperature for 1 hour. The mixture was quenched with saturated aq·NaHCO₃ solution and extracted with EtOAc twice. The combined organic layers were washed with brine, dried over with anhydrous Na₂SO₄, filtered, and concentrated to dryness under reduced pressure. The residue was purified by flash chromatography on silica gel (DCM:MeOH=15:1) to give (2S,4S*)—N—((R)-1-(4-(N-acetoxycarbamimidoyl)thiophen-2-yl)ethyl)-1-((4-(4-fluorophenoxy)benzoyl)glycyl)-4-(methoxymethyl)pyrrolidine-2-carboxamide (23 mg, yield 38.7%) as a white solid. LCMS (ESI) m/z: 640 (M+H)⁺.

### Step 8: (2S,4S)—N—((R)-1-(4-carbamimidoylthiophen-2-yl)ethyl)-1-((4-(4-fluorophenoxy)benzoyl)glycyl)-4-(methoxymethyl)pyrrolidine-2-carboxamide (Compound 80)

To a solution of (2S,4S*)—N—((R)-1-(4-(N-acetoxycarbamimidoyl)thiophen-2-yl)ethyl)-1-((4-(4-fluorophenoxy)benzoyl)glycyl)-4-(methoxymethyl)pyrrolidine-2-carboxamide (23 mg, 0.036 mmol) in MeOH (2 mL) was added Pd/C (5 mg, 10% wt.) at room temperature, the mixture was degassed under N₂ atmosphere for three times and stirred under a H₂ balloon at room temperature for 16 hours. The mixture was filtered, and the filtrate was concentrated under reduced pressure to dryness. The residue was purified by flash chromatography on silica gel (DCM:MeOH=15:1) and further purified by prep-HPLC to give Compound 80 (3.5 mg, yield 16.7%) as a white solid. ¹H NMR (400 MHz, CD₃OD) δ 8.55 (s, 1H), 8.23 (t, J=3.9 Hz, 1H), 7.85 (d, J=8.8 Hz, 2H), 7.53 (s, 1H), 7.15 (dd, J=11.6, 5.8 Hz, 2H), 7.10-7.07 (m, 2H), 6.99 (d, J=8.8 Hz, 2H), 5.28-5.23 (m, 1H), 4.43 (t, J=8.2 Hz, 1H), 4.27 (d, J=16.5 Hz, 1H), 4.10 (d, J=16.5 Hz, 1H), 3.96-3.91 (m, 1H), 3.46-3.40 (m, 3H), 3.33 (s, 3H), 2.63 (d, J=9.2 Hz, 1H), 2.47-2.34 (m, 1H), 1.80-1.71 (m, 1H), 1.57 (d, J=7.0 Hz, 3H). LC/MS (ESI) m/z: 582 (M+H)⁺.

### Scheme 80: Synthesis of (2S,4S)—N—((R)-1-(4-carbamimidoylthiophen-2-yl)ethyl)-1-((9,9-difluoro-9H-fluorene-3-carbonyl)glycyl)-4-phenylpyrrolidine-2-carboxamide (Compound 81)

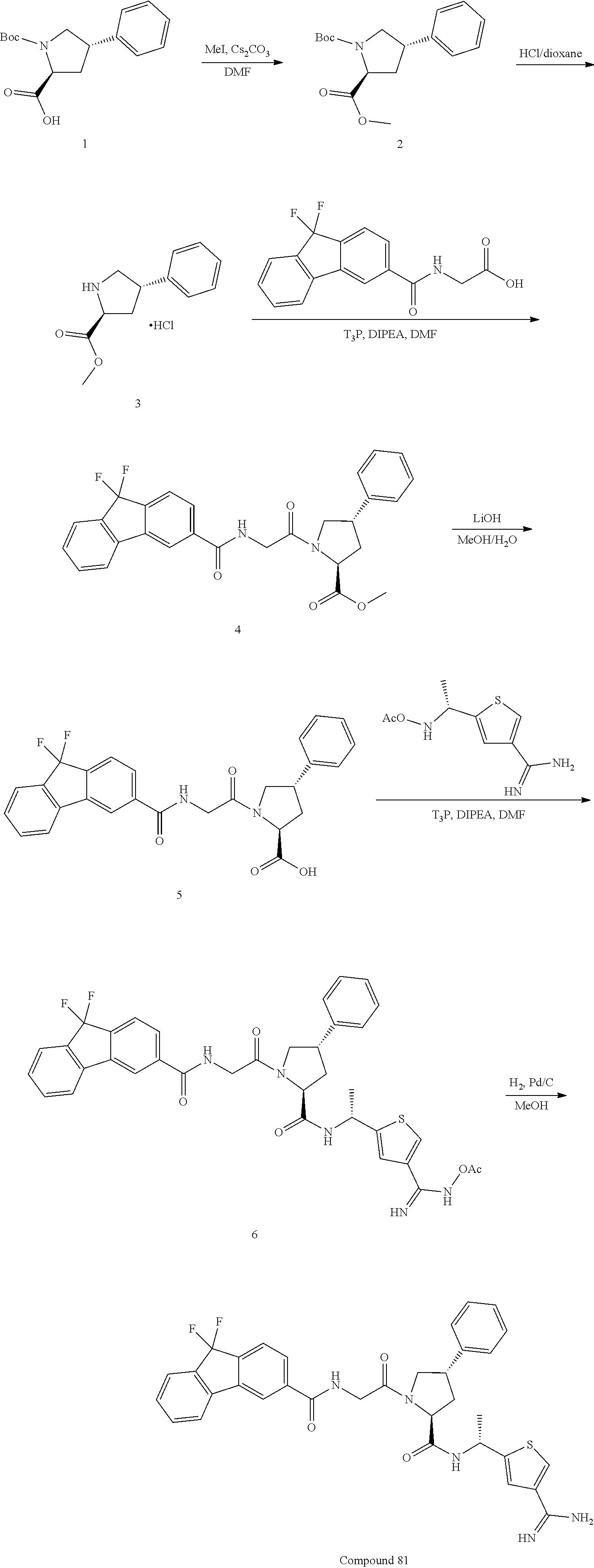

### Step 1: 1-(tert-butyl) 2-methyl (2S,4S)-4-phenylpyrrolidine-1,2-dicarboxylate (2)

To a solution of (2S,4S)-1-(tert-butoxycarbonyl)-4-phenylpyrrolidine-2-carboxylic acid (170 mg, 0.58 mmol) in DMF (1.5 mL) was added Cs₂CO₃ (554 mg, 1.7 mmol) and MeI (170 mg, 1.2 mmol) and the mixture was stirred at room temperature overnight. The mixture was diluted with EtOAc, washed with water and brine, dried over anhydrous Na₂SO₄, filtered, and concentrated under reduced pressure to dryness. The residue was purified by column chromatography on silica gel (PE:EtOAc=2:1) to give 1-(tert-butyl) 2-methyl (2S,4S)-4-phenylpyrrolidine-1,2-dicarboxylate (130 mg, yield 73.5%) as a colorless oil. LCMS (ESI) m/z: 306 (M+H)⁺.

### Step 2: methyl (2S,4S)-4-phenylpyrrolidine-2-carboxylate hydrochloride (3)

A solution of 1-(tert-butyl) 2-methyl (2S,4S)-4-phenylpyrrolidine-1,2-dicarboxylate (130 mg, 0.43 mmol) in HCl/1,4-dioxane (2 mL) was stirred at room temperature for 30 minutes. The reaction mixture was concentrated under reduced pressure to dryness to give methyl (2S,4S)-4-phenylpyrrolidine-2-carboxylate (110 mg, yield 100%) as a light-yellow solid, which was used directly in the next reaction without further purification. LC/MS (ESI) m/z: 206 (M+H)⁺.

### Step 3: methyl (2S,4S)-1-((9,9-difluoro-9H-fluorene-3-carbonyl)glycyl)-4-phenylpyrrolidine-2-carboxylate (4)

To a mixture of methyl (2S,4S)-4-phenylpyrrolidine-2-carboxylate hydrochloride (110 mg, 0.43 mmol) and (9,9-difluoro-9H-fluorene-3-carbonyl)glycine (127 mg, 0.42 mmol) in DMF (2 mL) was added DIPEA (325 mg, 2.5 mmol) and T₃P (350 mg, 0.55 mmol, 50% wt. in EtOAc) at 0° C. under N₂ atmosphere and the mixture was stirred at 25° C. for 2 hours. The mixture was diluted with EtOAc and washed with water and brine, dried over anhydrous Na₂SO₄, filtered, and concentrated under reduced pressure to dryness. The residue was purified by column chromatography on silica gel (PE:EtOAc=1:1) to give methyl (2S,4S)-1-((9,9-difluoro-9H-fluorene-3-carbonyl)glycyl)-4-phenylpyrrolidine-2-carboxylate (170 mg, yield 82.6%) as a colorless oil. LC/MS (ESI) m/z: 491 (M+H)⁺.

### Step 4: (2S,4S)-1-((9,9-difluoro-9H-fluorene-3-carbonyl)glycyl)-4-phenylpyrrolidine-2-carboxylic acid (5)

To a solution of methyl (2S,4S)-1-((9,9-difluoro-9H-fluorene-3-carbonyl)glycyl)-4-phenylpyrrolidine-2-carboxylate (170 mg, 0.35 mmol) in MeOH (2 mL) and water (1 mL) was added LiOH·H₂O (86 mg, 2.1 mmol) at 0° C. and the mixture was stirred at room temperature for 2 hours. The mixture was acidified with 1 N aq·HCl to pH-3, extracted with EtOAc twice. The combined organic layers were washed with brine, dried over anhydrous Na₂SO₄, filtered, and concentrated under reduced pressure to dryness to give (2S,4S)-1-((9,9-difluoro-9H-fluorene-3-carbonyl)glycyl)-4-phenylpyrrolidine-2-carboxylic acid (150 mg, yield 90.0%) as a colorless oil, which was used directly in the next step. LC/MS (ESI) (m/z): 477 (M+H)⁺.

### Step 5: (2S,4S)—N—((R)-1-(4-(N-acetoxycarbamimidoyl)thiophen-2-yl)ethyl)-1-((9,9-difluoro-9H-fluorene-3-carbonyl)glycyl)-4-phenylpyrrolidine-2-carboxamide (6)

To a mixture of (2S,4S)-1-((9,9-difluoro-9H-fluorene-3-carbonyl)glycyl)-4-phenylpyrrolidine-2-carboxylic acid (50 mg, 0.10 mmol) and (R)-5-(1-(acetoxyamino)ethyl)thiophene-3-carboximidamide (27 mg, 0.12 mmol) in DMF (2 mL) was added DIPEA (78 mg, 0.60 mmol) and T₃P (82 mg, 0.13 mmol, 50% wt. in EtOAc) at 0° C. under N₂ atmosphere and the mixture was stirred at 25° C. for 2 hours. The mixture was diluted with EtOAc and washed with water. The aqueous layer was extracted with EtOAc twice and the combined organic layers were dried over anhydrous Na₂SO₄, filtered, and concentrated under reduced pressure to dryness. The residue was purified by column chromatography on silica gel (DCM:MeOH=15:1) to give (2S,4S)—N—((R)-1-(4-(N-acetoxycarbamimidoyl)thiophen-2-yl)ethyl)-1-((9,9-difluoro-9H-fluorene-3-carbonyl)glycyl)-4-phenylpyrrolidine-2-carboxamide (27 mg, yield 39.0%) as a colorless oil. LC/MS (ESI) m/z: 491 (M+H)⁺.

### Step 6: (2S,4S)—N—((R)-1-(4-carbamimidoylthiophen-2-yl)ethyl)-1-((9,9-difluoro-9H-fluorene-3-carbonyl)glycyl)-4-phenylpyrrolidine-2-carboxamide (Compound 61)

To a solution of (2S,4S)—N—((R)-1-(4-(N-acetoxycarbamimidoyl)thiophen-2-yl)ethyl)-1-((9,9-difluoro-9H-fluorene-3-carbonyl)glycyl)-4-phenylpyrrolidine-2-carboxamide (27 mg, 0.039 mmol) in MeOH (2 mL) was added Pd/C (5 mg, 10% wt.) at room temperature and the mixture was stirred under a H₂ balloon at room temperature overnight. The mixture was filtered, and the filtrate was concentrated to dryness. The residue was purified by prep-HPLC to give Compound 81 (1.8 mg, yield 7.4%) as a white solid. ¹H NMR (400 MHz, CD₃OD) δ 8.24 (t, J=6.7 Hz, 1H), 8.20 (s, 1H), 7.91-7.86 (m, 1H), 7.74 (dd, J=17.7, 7.7 Hz, 2H), 7.65 (d, J=7.5 Hz, 1H), 7.58 (t, J=7.5 Hz, 1H), 7.53 (d, J=15.8 Hz, 1H), 7.45 (t, J=7.5 Hz, 1H), 7.38-7.31 (m, 4H), 7.27 (dd, J=10.0, 4.3 Hz, 1H), 5.35-5.31 (m, 1H), 4.70 (d, J=6.7 Hz, 1H), 4.25 (d, J=27.8 Hz, 2H), 3.70 (t, J=20.1 Hz, 2H), 2.53-2.33 (m, 2H), 2.03 (s, 1H), 1.63 (t, J=17.0 Hz, 3H). LC/MS (ESI) (m/z): 628 (M+H)⁺.

### Scheme 81: Synthesis of (S)—N—((R)-1-(4-carbamimidoylthiophen-2-yl)ethyl)-7-((4-(difluoro(phenyl)methyl)benzoyl)glycyl)-1,4-dioxa-7-azaspiro[4.4]nonane-8-carboxamide (Compound 82)

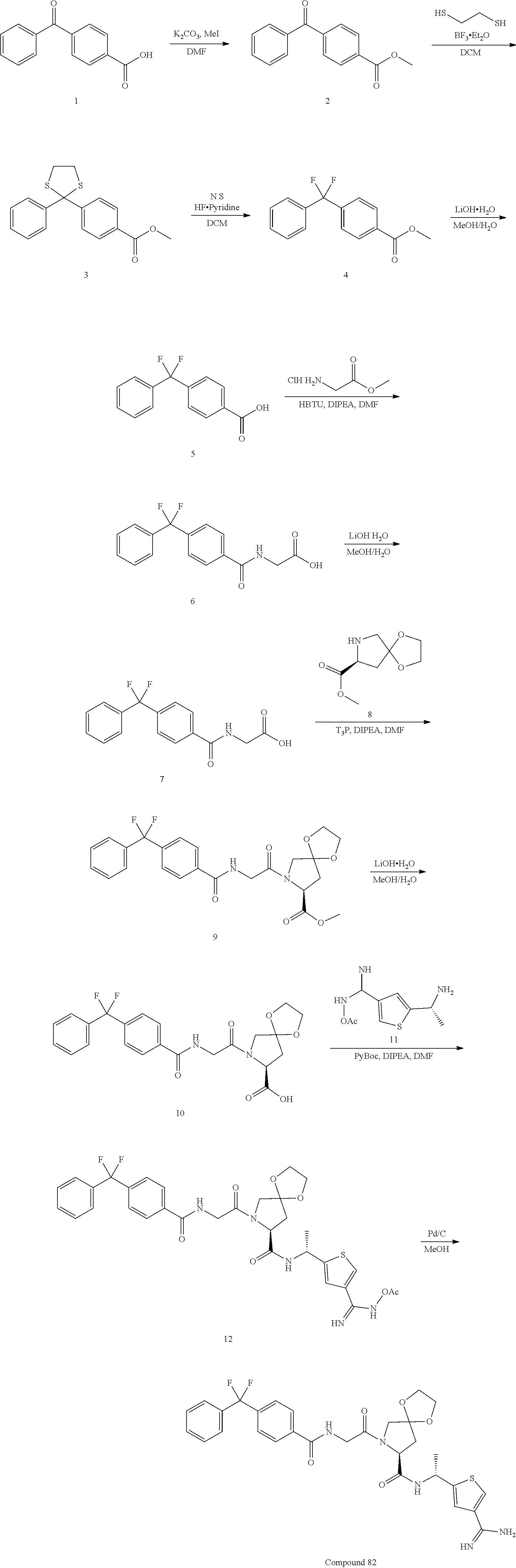

### Step 1: methyl 4-benzoylbenzoate (2)

To a mixture of 4-benzoylbenzoic acid (2.0 g, 8.84 mmol) in DMF (20 mL) was added K₂CO₃ (3.66 g, 26.52 mmol) and MeI (2.51 g, 17.68 mmol) and the mixture was stirred at 25° C. overnight. The mixture was diluted with EtOAc, washed with water and brine, dried over anhydrous Na₂SO₄, filtered, and concentrated under reduced pressure to dryness. The residue was purified by flash chromatography (PE:EtOAc=3:1) to give methyl 4-benzoylbenzoate (2.1 g, yield 98.9%) as a white solid. LC/MS (ESI) m/z: 241 (M+H)⁺.

### Step 2: methyl 4-(2-phenyl-1,3-dithiolan-2-yl)benzoate (3)

To a solution of methyl 4-benzoylbenzoate (2.0 g, 8.32 mmol) in DCM (20 mL) was added ethane-1,2-dithiol (1.57 g, 16.64 mmol) and BF₃·Et₂O (20 mL) at 0° C. The mixture was stirred at room temperature under N₂ atmosphere for 4 hours. The mixture was diluted with water and extracted with DCM (2×30 mL). The combined organic layers were washed with brine, dried over anhydrous Na₂SO₄, filtered, and concentrated under reduced pressure to dryness. The residue was purified by flash chromatography on silica gel (PE:EtOAc=50:1) to give methyl 4-(2-phenyl-1,3-dithiolan-2-yl)benzoate (2.16 g, yield 82.1%) as a white solid.

### Step 3: methyl 4-(difluoro(phenyl)methyl)benzoate (4)

To a solution of NIS (5.74 g, 33.2 mmol) in DCM (20 mL) was added HF-pyridine (15 mL) at 0° C. and the mixture was stirred for 10 minutes. Then a solution of methyl 4-(2-phenyl-1,3-dithiolan-2-yl)benzoate (2.1 g, 6.64 mmol) in DCM (20 mL) was added to the mixture drop-wisely and the resulting mixture was stirred at room temperature for 2 hours. The reaction mixture was quenched with water and extracted with DCM (2×30 mL). The combined organic layers were washed with brine, dried over anhydrous Na₂SO₄, filtered, and concentrated under reduced pressure to dryness. The residue was purified by flash chromatography on silica gel (PE:EtOAc=50:1) to methyl 4-(difluoro(phenyl)methyl)benzoate (1.68 g, yield 96.5%) as a white solid.

### Step 4: 4-(difluoro(phenyl)methyl)benzoic acid (5)

To a solution of methyl 4-(difluoro(phenyl)methyl)benzoate (1.6 g, 6.1 mmol) in THF (8 mL), MeOH (8 mL) and water (2 mL) was added a solution of LiOH·H₂O (770 mg, 18.3 mmol) at 0° C. and the mixture was stirred at room temperature for 16 hours. The mixture was acidified with 1 N aq·HCl solution to pH-3 and extracted with EtOAc twice. The combined organic layers were washed with brine, dried over anhydrous Na₂SO₄, filtered, and concentrated under reduced pressure to dryness to give 4-(difluoro(phenyl)methyl)benzoic acid (1.49 g, yield 98.4%) as a white solid, which was used directly in the next step without further purification. LC/MS (ESI) (m/z): 249 (M+H)⁺.

### Step 5: methyl (9,9-difluoro-9H-fluorene-3-carbonyl)glycinate (6)

To a solution of 4-(difluoro(phenyl)methyl)benzoic acid (1.42 g, 5.72 mmol) in DMF (15 mL) was added methyl glycinate hydrochloride (1.08 g, 8.58 mmol), HBTU (2.6 g, 6.86 mmol) and DIPEA (2.22 g, 17.16 mmol) under N₂ atmosphere and the reaction mixture was stirred at room temperature for 1 hour. The mixture was diluted with EtOAc and washed with saturated aq·NH₄Clsolution and brine, dried over anhydrous Na₂SO₄, filtered, and concentrated to dryness. The aqueous layer was extracted with EtOAc twice and the combined organic layers were washed with brine, dried over anhydrous Na₂SO₄, filtered, and concentrated under reduced pressure to dryness. The residue was purified by flash chromatography on silica gel (PE:EtOAc=5:1) to afford methyl (4-(difluoro(phenyl)methyl)benzoyl)glycinate (1.72 g, yield 94.2%) as a white solid. LC/MS (ESI) m/z: 320 (M+H)⁺.

### Step 6: (4-(difluoro(phenyl)methyl)benzoyl)glycine (7)

At 0° C., to a mixture of methyl (4-(difluoro(phenyl)methyl)benzoyl)glycinate (1.7 g, 5.32 mmol) in MeOH/H₂O (20 mL, 4/1) was added LiOH·H₂O (670 mg, 16 mmol) and the reaction mixture was stirred at room temperature for 4 hours. The mixture was acidified with 1 N aq·HCl to pH-4 and extracted with EtOAc twice. The combined organic layers were washed with brine, dried over anhydrous Na₂SO₄, filtered, and concentrated under reduced pressure to dryness to afford (4-(difluoro(phenyl)methyl)benzoyl)glycine (1.45 g, yield 89.3%) as a white solid. LC/MS (ESI) m/z: 306 (M+H)⁺.

### Step 7: methyl (S)-7-((4-(difluoro(phenyl)methyl)benzoyl)glycyl)-1,4-dioxa-7-azaspiro[4.4]nonane-8-carboxylate (9)

To a solution of methyl (4-(difluoro(phenyl)methyl)benzoyl)glycinate (92 mg, 0.30 mmol), methyl (S)-1,4-dioxa-7-azaspiro[4.4]nonane-8-carboxylate (85 mg, 0.45 mmol) and DIPEA (130 mg, 1.0 mmol) in DMF (1 mL) was added T₃P (287 mg, 0.45 mmol, 50% wt. in EtOAc) under N₂ atmosphere and the reaction mixture was stirred at room temperature for 1 hour. The mixture was diluted with EtOAc and washed with saturated aq·NaHCO₃ solution. The aqueous layer was extracted with EtOAc twice and the combined organic layers were washed with brine, dried over anhydrous Na₂SO₄, filtered, and concentrated under reduced pressure to dryness. The residue was purified by flash chromatography on silica gel (PE:EtOAc=3:1) to afford methyl (S)-7-((4-(difluoro(phenyl)methyl)benzoyl)glycyl)-1,4-dioxa-7-azaspiro[4.4]nonane-8-carboxylate (100 mg, yield 70.3%) as a yellow oil. LC/MS (ESI) m/z: 475 (M+H)⁺.

### Step 8: (S)-7-((4-(difluoro(phenyl)methyl)benzoyl)glycyl)-1,4-dioxa-7-azaspiro[4.4]nonane-6-carboxylic acid (10)

To a solution of methyl (S)-7-((4-(difluoro(phenyl)methyl)benzoyl)glycyl)-1,4-dioxa-7-azaspiro[4.4]nonane-8-carboxylate (90 mg, 0.19 mmol) in MeOH/H₂O (2.5 mL, 4/1) was added LiOH·H₂O (24 mg, 0.57 mmol) and the reaction mixture was stirred at room temperature for 4 hours. The mixture was acidified with 1 N aq·HCl to pH˜4 and extracted with EtOAc twice. The combined organic layers were washed with brine, dried over anhydrous Na₂SO₄, filtered, and concentrated under reduced pressure to dryness to afford (S)-7-((4-(difluoro(phenyl)methyl)benzoyl)glycyl)-1,4-dioxa-7-azaspiro[4.4]nonane-8-carboxylic acid (75 mg, yield 85.7%) as a white solid. LC/MS (ESI) m/z: 461 (M+H)⁺.

### Step 9: (S)—N—((R)-1-(4-(N-acetoxycarbamimidoyl)thiophen-2-yl)ethyl)-7-((4-(difluoro(phenyl)methyl)benzoyl)glycyl)-1,4-dioxa-7-azaspiro[4.4]nonane-6-carboxamide (12)

At 0° C., to a mixture of (S)-7-((4-(difluoro(phenyl)methyl)benzoyl)glycyl)-1,4-dioxa-7-azaspiro[4.4]nonane-8-carboxylic acid (75 mg, 0.16 mmol), (R)—N-acetoxy-5-(1-aminoethyl)thiophene-3-carboximidamide (73 mg,0.32 mmol) and DIPEA (62 mg, 0.48 mmol) in DMF (1 mL) was added PyBop (100 mg, 0.19 mmol) under N₂ atmosphere and the reaction mixture was stirred at room temperature for 1 hour. The mixture was diluted with EtOAc, washed with saturated aq·NH₄Cl solution and brine, dried over anhydrous Na₂SO₄, filtered, and concentrated under reduced pressure to dryness. The residue was purified by flash chromatography on silica gel (PE:EtOAc=1:1) to afford (S)—N—((R)-1-(4-(N-acetoxycarbamimidoyl)thiophen-2-yl)ethyl)-7-((4-(difluoro(phenyl)methyl)benzoyl)glycyl)-1,4-dioxa-7-azaspiro[4.4]nonane-8-carboxamide (70 mg, yield 55.0%) as a yellow solid. LC/MS (ESI) m/z: 670 (M+H)⁺.

### Step 10: (S)—N—((R)-1-(4-carbamimidoylthiophen-2-yl)ethyl)-7-((4-(difluoro(phenyl) methyl)benzoyl)glycyl)-1,4-dioxa-7-azaspiro[4.4]nonane-6-carboxamide (Compound 82)

To a mixture of (S)—N—((R)-1-(4-(N-acetoxycarbamimidoyl)thiophen-2-yl)ethyl)-7-((4-(difluoro(phenyl)methyl)benzoyl)glycyl)-1,4-dioxa-7-azaspiro[4.4]nonane-8-carboxamide (67 mg, 0.1 mmol) in MeOH (5 mL) was added Pd/C (10 mg, 10% wt.) under N₂ atmosphere, the mixture was degassed under N₂ atmosphere for three times and stirred under a H₂ balloon at room temperature overnight. The mixture was filtered, and the filtrate was concentrated under reduced pressure to dryness. The residue was purified by prep-HPLC to give Compound 82 (23.5 mg, yield 38.4%) as a white solid. ¹HNMR (400 MHz, CD₃OD) δ 8.53 (s, 1H), 8.23 (dd, J=8.2, 1.5 Hz, 1H), 7.92 (d, J=8.4 Hz, 2H), 7.61 (d, J=8.4 Hz, 2H), 7.56-7.49 (m, 3H), 7.48-7.41 (m, 3H), 5.36-5.23 (m, 1H), 4.55 (dd, J=8.8, 6.6 Hz, 1H), 4.21-4.08 (m, 2H), 4.02-3.93 (m, 4H), 3.77 (q, J=10.7 Hz, 2H), 2.42 (dd, J=13.2, 8.9 Hz, 1H), 2.19 (dd, J=13.2, 6.6 Hz, 1H), 1.56 (d, J=7.0 Hz, 3H). LC/MS (ESI) m/z: 612 (M+H)⁺.

### Scheme 82: (S)—N-((4-carbamimidoylthiophen-2-yl)methyl)-7-((3-fluoro-[1,1′-biphenyl]-4-carbonyl)glycyl)-1,4-dioxa-7-azaspiro[4.4]nonane-8-carboxamide (Compound 83)

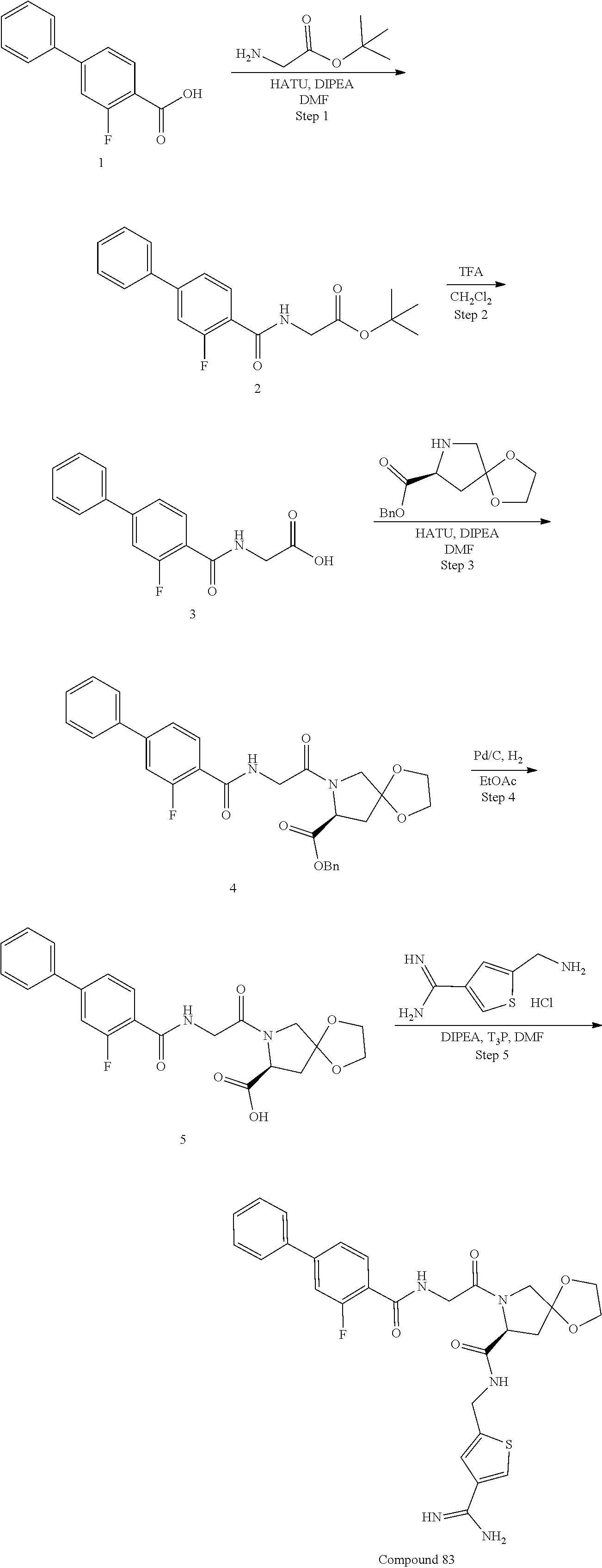

Compound 83 was prepared as a white solid from 2-fluoro-4-phenyl-benzoic acid, tert-butyl 2-aminoacetate·HCl, benzyl (1 S,3S,5S)-5-methyl-2-azabicyclo[3.1.0]hexane-3-carboxylate, and 5-(aminomethyl)thiophene-3-carboximidamide. HCl based on the procedures set forth in Scheme 1. LC/MS (ESI) m/z: 566 (M+H)⁺. ¹H NMR (400 MHz, DMSO-d₆) δ 8.70 (s, 1H), 8.54-8.16 (m, 2H), 7.93-7.74 (m, 2H), 7.65 (t, J=9.9 Hz, 3H), 7.51 (t, J=7.4 Hz, 1H), 7.44 (t, J=7.2 Hz, 1H), 4.59-4.36 (m, 4H), 4.27-4.01 (m, 2H), 3.91 (dt, J=17.9, 6.4 Hz, 4H), 3.82-3.67 (m, 1H), 3.61 (d, J=10.8 Hz, 1H), 3.47-3.29 (m, 2H), 2.39-2.24 (m, 1H), 2.22 (d, J=9.3 Hz, 1H), 2.17-1.93 (m, 1H).

### Scheme 83: Synthesis of (1S,3S,5R)—N—((R)-1-(4-carbamimidoylthiophen-2-yl)ethyl)-2-((4-(4-fluorophenoxy)benzoyl)glycyl)-5-((2-(2-(((2S,3S,4S,5S)-2,3,4,5-tetrahydroxyhexyl)amino)acetamido)ethoxy)methyl)-2-azabicyclo[3.1.0]hexane-3-carboxamide (Compound 84)

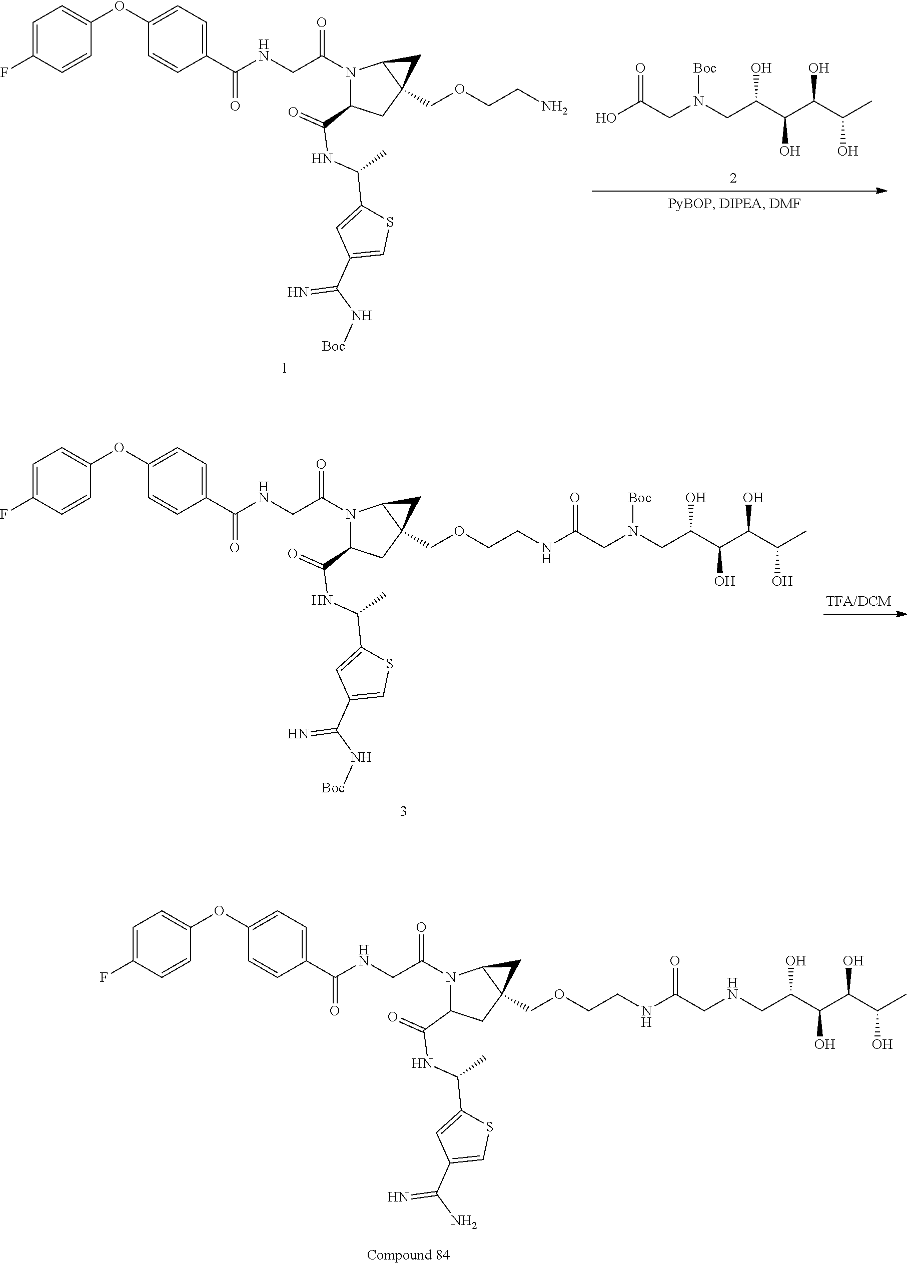 [SMILES: C[C@@H](NC(=O)[C@@H]1C[C@]2(COCCN)C[C@@H]2N1C(=O)CNC(=O)c1ccc(Oc2ccc(F)cc2)cc1)c1cc(C(=N)NC(=O)OC(C)(C)C)cs1 ; C[C@H](O)[C@H](O)[C@@H](O)[C@@H](O)CN(CC(=O)O)C(=O)OC(C)(C)C ; C[C@H](O)[C@H](O)[C@@H](O)[C@@H](O)CN(CC(=O)NCCOC[C@@]12C[C@@H]1N(C(=O)CNC(=O)c1ccc(Oc3ccc(F)cc3)cc1)[C@H](C(=O)N[C@H](C)c1cc(C(=N)NC(=O)OC(C)(C)C)cs1)C2)C(=O)OC(C)(C)C ; C[C@H](O)[C@H](O)[C@@H](O)[C@@H](O)CNCC(=O)NCCOC[C@]12CC(C(=O)N[C@H](C)c3cc(C(=N)N)cs3)N(C(=O)CNC(=O)c3ccc(Oc4ccc(F)cc4)cc3)[C@H]1C2]

### Step 1: tert-butyl (2-((2-(((1S,3S,5R)-3-(((R)-1-(4-(N-(tert-butoxycarbonyl)carbamimidoyl) thiophen-2-yl)ethyl)carbamoyl)-2-((4-(4-fluorophenoxy)benzoyl)glycyl)-2-azabicyclo[3.1.0]hexan-5-yl)methoxy)ethyl)amino)-2-oxoethyl)((2S,3S,4S,5S)-2,3,4,5-tetrahydroxyhexyl) carbamate (3)

To a mixture of tert-butyl ((5-((R)-1-((1S,3S,5R)-5-((2-aminoethoxy)methyl)-2-((4-(4-fluorophenoxy)benzoyl)glycyl)-2-azabicyclo[3.1.0]hexane-3-carboxamido)ethyl)thiophen-3-yl)(imino)methyl)carbamate (150 mg, 0.21 mmol) and N-(tert-butoxycarbonyl)-N-((2S,3S,4S,5S)-2,3,4,5-tetrahydroxyhexyl)glycine (100 mg, 0.32 mmol) in DMF (3.0 mL) was added DIPEA (0.17 mL,1.05 mmol) and PyBOP (162 mg, 0.32 mmol) at 0° C. under N₂ atmosphere and the mixture was stirred at 25° C. for 1 hour. The mixture was concentrated to dryness under reduced pressure. The residue was purified by prep-TLC (DCM:MeOH=10:1) and further purified by prep-HPLC to give tert-butyl (2-((2-(((1 S,3S,5R)-3-(((R)-1-(4-(N-(tert-butoxycarbonyl)carbamimidoyl)thiophen-2-yl)ethyl)carbamoyl)-2-((4-(4-fluorophenoxy)benzoyl)glycyl)-2-azabicyclo[3.1.0]hexan-5-yl)methoxy)ethyl)amino)-2-oxoethyl)((2S,3S,4S,5S)-2,3,4,5-tetrahydroxyhexyl)carbamate (60 mg, yield 28.2%) as a white solid. LC/MS (ESI) m/z: 1028 (M+H)⁺.

### Step 2: (1S,3S,5R)—N—((R)-1-(4-carbamimidoylthiophen-2-yl)ethyl)-2-((4-(4-fluorophenoxy) benzoyl)glycyl)-5-((2-(2-(((2S,3S,4S,5S)-2,3,4,5-tetrahydroxyhexyl)amino)acetamido)ethoxy) methyl)-2-azabicyclo[3.1.0]hexane-3-carboxamide (Compound 84)

To a solution of tert-butyl (2-((2-(((1 S,3S,5R)-3-(((R)-1-(4-(N-(tert-butoxycarbonyl) carbamimidoyl)thiophen-2-yl)ethyl)carbamoyl)-2-((4-(4-fluorophenoxy)benzoyl)glycyl)-2-azabicyclo[3.1.0]hexan-5-yl)methoxy)ethyl)amino)-2-oxoethyl)((2S,3S,4S,5S)-2,3,4,5-tetrahydroxyhexyl)carbamate (60 mg, 0.06 mmol) in DCM (3 mL) was added TFA (1 mL) at 0° C., and the reaction mixture was stirred at 25° C. for 1 hour. The mixture was concentrated to dryness under reduced pressure. The residue was purified by prep-HPLC to give Compound 84 (25 mg, yield 52.1%) as a white solid. ¹HNMR (400 MHz, CD₃OD) δ 8.47 (s, 1H), 8.23 (t, J=4.0 Hz, 1H), 7.85 (d, J=8.8 Hz, 2H), 7.51 (d, J=6.4 Hz, 1H), 7.19-7.14 (m, 2H), 7.12-7.07 (m, 2H), 7.00 (d, J=8.8 Hz, 2H), 5.23 (q, J=6.9 Hz, 1H), 4.84 (d, J=3.7 Hz, 1H), 4.45 (d, J=16.6 Hz, 1H), 4.25 (d, J=16.6 Hz, 1H), 3.87 (dd, J=7.6, 3.5 Hz, 1H), 3.82-3.75 (m, 2H), 3.68-3.55 (m, 6H), 3.45 (dd, J=11.4, 6.4 Hz, 4H), 3.25 (d, J=12.6 Hz, 1H), 3.01 (dd, J=12.3, 7.9 Hz, 1H), 2.70 (t, J=12.4 Hz, 1H), 2.10 (dd, J=13.4, 3.7 Hz, 1H), 1.61 (dd, J=24.3, 7.0 Hz, 3H), 1.31-1.24 (m, 4H), 0.99 (t, J=5.9 Hz, 1H). LC/MS (ESI) m/z: 828 (M+H)⁺.

### Scheme 84: Synthesis of (S)—N—((R)-1-(5-carbamimidoylthiophen-2-yl)ethyl)-7-((4-(4-fluorophenoxy)benzoyl)glycyl)-1,4-dioxa-7-azaspiro[4.4]nonane-8-carboxamide (Compound 85)

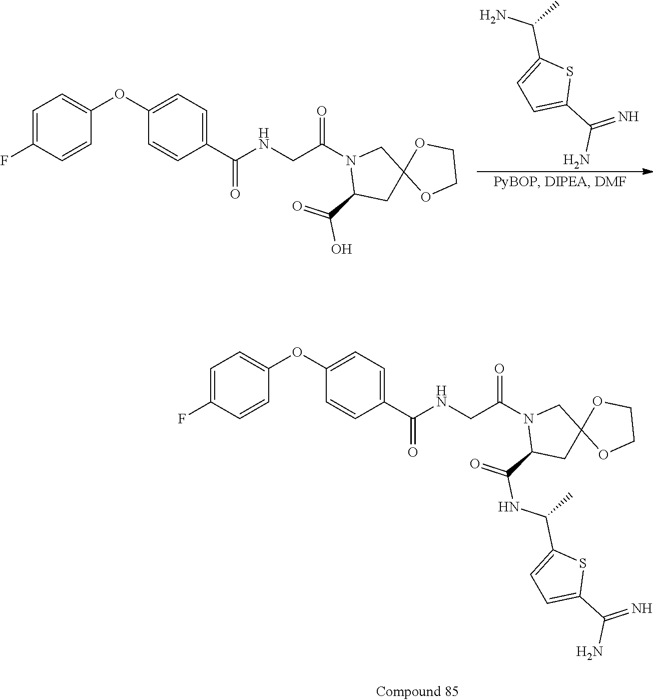

Compound 85 was prepared as a white solid from (S)-7-((4-(4-fluorophenoxy)benzoyl)glycyl)-1,4-dioxa-7-azaspiro[4.4]nonane-8-carboxylic acid and (R)-5-(1-aminoethyl)thiophene-2-carboximidamide based on the procedures set forth in Scheme 3. ¹H NMR (400 MHz, CD₃OD) δ 8.52 (s, 1H), 7.86 (d, J=8.8 Hz, 2H), 7.79 (d, J=4.0 Hz, 1H), 7.25 (d, J=4.0 Hz, 1H), 7.18-7.13 (m, 2H), 7.10-7.06 (m, 2H), 7.00-6.97 (m, 2H), 5.37-5.28 (m, 1H), 4.55 (dd, J=8.7, 6.8 Hz, 1H), 4.17 (t, J=15.1 Hz, 2H), 3.99 (dd, J=8.3, 3.5 Hz, 4H), 3.76 (t, J=9.3 Hz, 2H), 2.43 (dd, J=13.1, 9.0 Hz, 1H), 2.20 (dd, J=13.2, 6.6 Hz, 1H), 1.60 (t, J=16.8 Hz, 3H). LC/MS (ESI) m/z: 596 (M+H)⁺.

### Scheme 85: Synthesis of (2S,4R)—N—((R)-1-(4-(1H-imidazol-2-yl)thiophen-2-yl)ethyl)-4-(difluoromethoxy)-1-((4-(4-fluorophenoxy)benzoyl)glycyl)pyrrolidine-2-carboxamide (Compound 86)

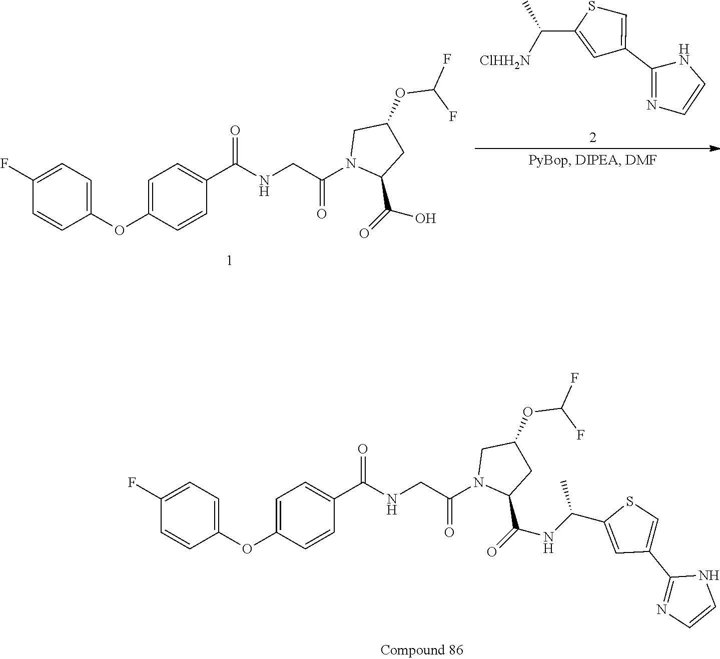

Compound 85 was prepared as a white solid from (2S,4R)-4-(difluoromethoxy)-1-((4-(4-fluorophenoxy)benzoyl)glycyl)pyrrolidine-2-carboxylic acid and (R)-1-(4-(1H-imidazol-2-yl)thiophen-2-yl)ethan-1-amine hydrochloride based on the procedures set forth in Scheme 3. ¹HNMR (400 MHz, CD₃OD) δ 8.22 (s, 1H), 7.81 (d, J=8.9 Hz, 2H), 7.75 (s, 1H), 7.53 (d, J=11.7 Hz, 1H), 7.19-7.05 (m, 6H), 7.00-6.92 (m, 2H), 6.50 (td, J=74.5, 17.3 Hz, 1H), 5.31 (dt, J=20.1, 6.6 Hz, 1H), 4.99 (d, J=30.9 Hz, 1H), 4.71-4.58 (m, 1H), 4.34-3.98 (m, 2H), 3.97-3.78 (m, 2H), 2.69-2.44 (m, 1H), 2.41-2.20 (m, 1H), 1.62 (dd, J=29.0, 6.9 Hz, 3H). LC/MS (ESI) m/z: 628 (M+H)⁺.

### Scheme 86: Synthesis of (S)—N—((R)-1-(4-(1H-imidazol-2-yl)thiophen-2-yl)ethyl)-7-((9,9-difluoro-9H-fluorene-3-carbonyl)glycyl)-1,4-dioxa-7-azaspiro[4.4]nonane-8-carboxamide (Compound 87)

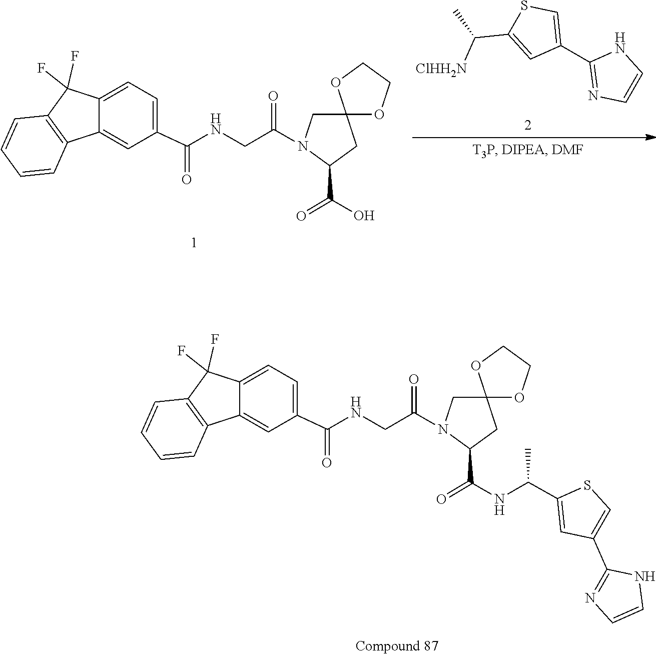

Compound 87 was prepared from (S)-7-((9,9-difluoro-9H-fluorene-3-carbonyl)glycyl)-1,4-dioxa-7-azaspiro[4.4]nonane-8-carboxylic acid and (R)-1-(4-(1H-imidazol-2-yl)thiophen-2-yl)ethan-1-amine hydrochloride based on the procedures set forth in Scheme 3. ¹HNMR (400 MHz, CD₃OD) δ 8.29 (s, 1H), 8.15 (s, 1H), 7.85 (t, J=6.6 Hz, 1H), 7.68 (dt, J=13.4, 7.5 Hz, 4H), 7.54 (dd, J=17.7, 9.5 Hz, 2H), 7.44 (t, J=7.5 Hz, 1H), 7.05 (d, J=25.9 Hz, 2H), 5.33 (dt, J=20.5, 7.3 Hz, 1H), 4.71-4.54 (m, 1H), 4.21 (dd, J=46.4, 16.8 Hz, 2H), 4.02-3.93 (m, 4H), 3.85-3.73 (m, 2H), 2.62-2.40 (m, 1H), 2.36-2.19 (m, 1H), 1.61 (t, J=17.7 Hz, 3H). LC/MS (ESI) m/z: 634 (M+H)⁺.

### Scheme 87: Synthesis of (S)—N-((4-carbamimidoylthiophen-2-yl)methyl)-7-((9-oxo-9H-fluorene-3-carbonyl)glycyl)-1,4-dioxa-7-azaspiro[4.4]nonane-8-carboxamide (Compound 88)

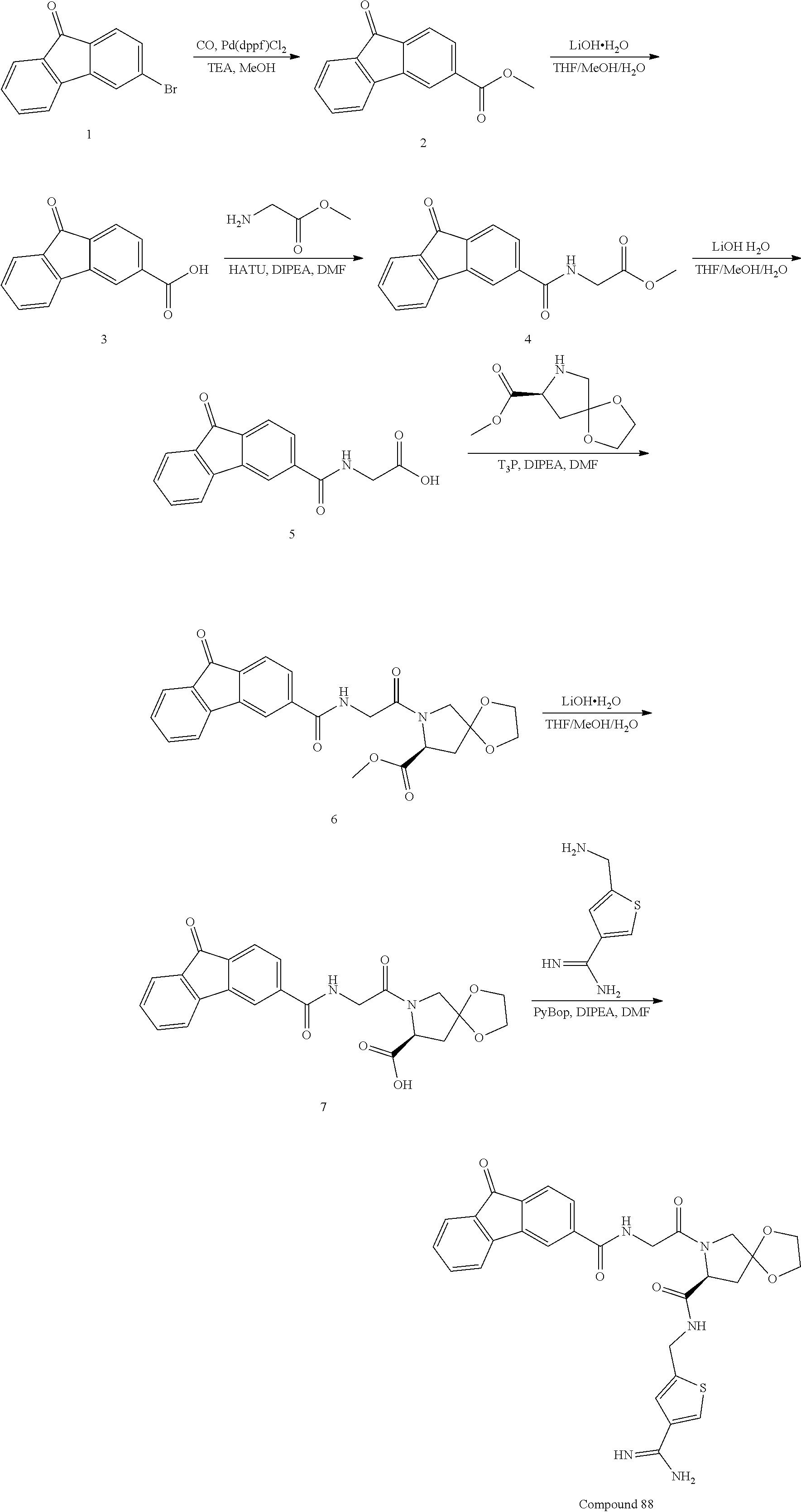

Compound 88 was prepared as a white solid from 3-bromo-9H-fluoren-9-one methyl glycinate, methyl (R)-1,4-dioxa-7-azaspiro[4.4]nonane-8-carboxylate, 5-(aminomethyl)thiophene-3-carboximidamide based on the procedures set forth in Scheme 21. ¹H NMR (400 MHz, CD₃OD) δ 8.54 (s, 1H), 8.26-8.08 (m, 2H), 7.80 (dd, J=7.7, 1.1 Hz, 1H), 7.74-7.60 (m, 4H), 7.52-7.39 (m, 2H), 4.65-4.55 (m, 3H), 4.19 (dt, J=16.6, 11.9 Hz, 2H), 4.04-3.95 (m, 4H), 3.85-3.74 (m, 2H), 2.61-2.41 (m, 1H), 2.37-2.20 (m, 1H). LC/MS (ESI) m/z: 574 (M+H)⁺.

### Scheme 88: Synthesis of (1S,3S,5R)—N—((R)-1-(4-carbamimidoylthiophen-2-yl) ethyl)-2-((9,9-difluoro-9H-fluorene-3-carbonyl)glycyl)-5-((2-(2-(((2S,3S,4S,5S)-2,3,4,5-tetrahydroxyhexyl)amino)acetamido)ethoxy)methyl)-2-azabicyclo[3.1.0]hexane-3-carboxamide (Compound 89)

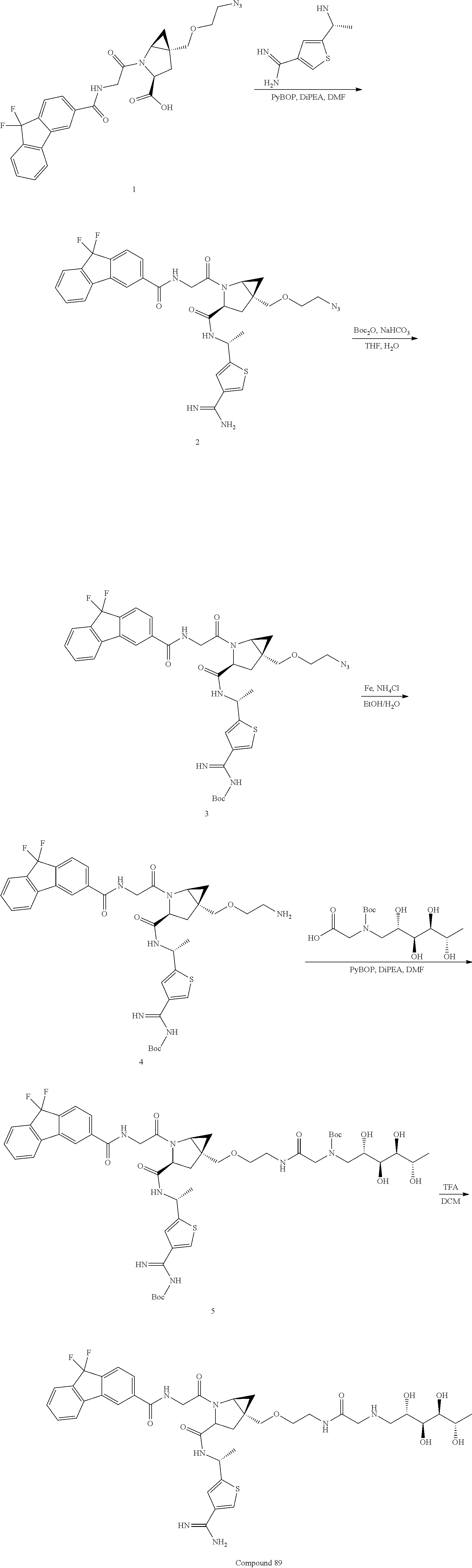

### Step 1: (1S,3S,5R)-5-((2-azidoethoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (2)

To a mixture of (1 S,3S,5R)-5-((2-azidoethoxy)methyl)-2-((9,9-difluoro-9H-fluorene-3-carbonyl) glycyl)-2-azabicyclo[3.1.0]hexane-3-carboxylic acid (602 mg, 1.18 mmol) and (R)-5-(1-aminoethyl)thiophene-3-carboximidamide hydrochloride (400 mg, 2.35 mmol) in DMF (6 mL) was added DIPEA (456 mg, 3.52 mmol) and PyBOP (917 mg, 1.76 mmol) at 0° C. under N₂ atmosphere and the mixture was stirred at 25° C. for 18 hours. The mixture was concentrated under reduced pressure to give (1S,3S,5R)-5-((2-azidoethoxy)ethyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (700 mg, crude) as a brown oil, which was directly used in the next reaction without purification. LC/MS (ESI) m/z: 663 (M+H)⁺.

### Step 2: tert-butyl ((5-((R)-1-((1S,3S,5R)-5-((2-azidoethoxy)methyl)-2-((9,9-difluoro-9H-fluorene-3-carbonyl)glycyl)-2-azabicyclo[3.1.0]hexane-3-carboxamido)ethyl)thiophen-3-yl)(imino)methyl)carbamate (3)

To a mixture of (1S,3S,5R)-5-((2-azidoethoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (700 mg, 1.06 mmol) and di-tert-butyl dicarbonate (345 mg, 1.58 mmol) in THF (7 mL) was added a solution of NaHCO₃ (266 mg, 3.18 mmol) in H₂O (2 mL) and the mixture was stirred at room temperature for 2 hours. The mixture was diluted with water and extracted with EtOAc twice. The combined organic layers were dried over anhydrous Na₂SO₄, filtered, and concentrated under reduced pressure. The residue was purified by prep-HPLC to give tert-butyl ((5-((R)-1-((1 S,3S,5R)-5-((2-azidoethoxy)methyl)-2-((9,9-difluoro-9H-fluorene-3-carbonyl)glycyl)-2-azabicyclo[3.1.0]hexane-3-carboxamido)ethyl)thiophen-3-yl)(imino)methyl)carbamate (170 mg, yield 21.0%) as a white solid. LC/MS (ESI) m/z: 763 (M+H)⁺.

### Step 3: tert-butyl ((5-((R)-1-((1S,3S,5R)-5-((2-aminoethoxy)methyl)-2-((9,9-difluoro-9H-fluorene-3-carbonyl)glycyl)-2-azabicyclo[3.1.0]hexane-3-carboxamido)ethyl)thiophen-3-yl)(imino)methyl)carbamate (4)

To a solution of tert-butyl ((5-((R)-1-((1 S,3S,5R)-5-((2-azidoethoxy)methyl)-2-((9,9-difluoro-9H-fluorene-3-carbonyl)glycyl)-2-azabicyclo[3.1.0]hexane-3-carboxamido)ethyl)thiophen-3-yl)(imino)methyl)carbamate (170 mg, 0.22 mmol) in EtOH (2 mL) and water (2 mL) was added Fe (74 mg, 1.32 mmol) and NH₄Cl (70 mg, 1.32 mmol) at room temperature and the mixture was stirred at 60° C. for 2 hours. The mixture was filtered, and the filter cake was washed with EtOAc twice. The filtrate was washed with water and brine, dried over anhydrous Na₂SO₄, filtered, and concentrated under reduced pressure to dryness. The residue was purified by prep-HPLC to give tert-butyl ((5-((R)-1-((1S,3S,5R)-5-((2-aminoethoxy)methyl)-2-((9,9-difluoro-9H-fluorene-3-carbonyl)glycyl)-2-azabicyclo[3.1.0]hexane-3-carboxamido)ethyl)thiophen-3-yl)(imino)methyl)carbamate (75 mg, yield 46.3%) as a white solid. LC/MS (ESI) m/z: 737 (M+H)⁺.

### Step 4: tert-butyl (2-((2-(((1S,3S,5R)-3-(((R)-1-(4-(N-(tert-butoxycarbonyl)carbamimidoyl) thiophen-2-yl)ethyl)carbamoyl)-2-((9,9-difluoro-9H-fluorene-3-carbonyl)glycyl)-2-azabicyclo[3.1.0]hexan-5-yl)methoxy)ethyl)amino)-2-oxoethyl)((2S,3S,4S,5S)-2,3,4,5-tetrahydroxyhexyl)carbamate (5)

To a mixture of tert-butyl ((5-((R)-1-((1S,3S,5R)-5-((2-aminoethoxy)methyl)-2-((9,9-difluoro-9H-fluorene-3-carbonyl)glycyl)-2-azabicyclo[3.1.0]hexane-3-carboxamido)ethyl)thiophen-3-yl)(imino)methyl)carbamate (56 mg, 0.073 mmol) and N-(tert-butoxycarbonyl)-N—((2S,3S,4S,5S)-2,3,4,5-tetrahydroxyhexyl)glycine (36 mg, 0.11 mmol) in DMF (2 mL) was added DIPEA (47 mg, 0.37 mmol) and PyBOP (57 mg, 0.11 mmol) at 0° C. under N₂ atmosphere and the mixture was stirred at 25° C. for 1 hour. The mixture was concentrated under reduced pressure to dryness. The residue was purified by prep-TLC (DCM:MeOH=10:1) and further purified by prep-HPLC to give tert-butyl (2-((2-(((1 S,3S,5R)-3-(((R)-1-(4-(N-(tert-butoxycarbonyl)carbamimidoyl)thiophen-2-yl)ethyl)carbamoyl)-2-((9,9-difluoro-9H-fluorene-3-carbonyl)glycyl)-2-azabicyclo[3.1.0]hexan-5-yl)methoxy)ethyl)amino)-2-oxoethyl)((2S,3S,4S,5S)-2,3,4,5-tetrahydroxyhexyl)carbamate (55 mg, yield 72.4%) as a white solid. LC/MS (ESI) m/z: 1042 (M+H)⁺.

### Step 5: (1S,3S,5R)—N—((R)-1-(4-carbamimidoylthiophen-2-yl)ethyl)-2-((9,9-difluoro-9H-fluorene-3-carbonyl)glycyl)-5-((2-(2-(((2S,3S,4S,5S)-2,3,4,5-tetrahydroxyhexyl)amino)acetamido)ethoxy)methyl)-2-azabicyclo[3.1.0]hexane-3-carboxamide (Compound 89)

To a solution of tert-butyl (2-((2-(((1 S,3S,5R)-3-(((R)-1-(4-(N-(tert-butoxycarbonyl) carbamimidoyl)thiophen-2-yl)ethyl)carbamoyl)-2-((9,9-difluoro-9H-fluorene-3-carbonyl)glycyl)-2-azabicyclo[3.1.0]hexan-5-yl)methoxy)ethyl)amino)-2-oxoethyl)((2S,3S,4S,5S)-2,3,4,5-tetrahydroxyhexyl)carbamate (55 mg, 0.053 mmol) in DCM (3 mL) was added TFA (1 mL) at 0° C. and the reaction mixture was stirred at 25° C. for 1 hour. The mixture was concentrated to dryness under reduced pressure. The residue was purified by prep-HPLC to give Compound 89 (13.4 mg, yield 30.0%) as a white solid. ¹H NMR (400 MHz, CD₃OD) δ 8.48 (s, 2H), 8.26-8.17 (m, 2H), 7.89 (d, J=7.8 Hz, 1H), 7.74 (dd, J=14.9, 7.8 Hz, 2H), 7.66 (d, J=7.4 Hz, 1H), 7.58 (t, J=7.7 Hz, 1H), 7.52 (d, J=8.1 Hz, 1H), 7.46 (t, J=7.5 Hz, 1H), 5.24 (q, J=7.5 Hz, 1H), 4.52 (d, J=16.4 Hz, 1H), 4.31 (d, J=16.5 Hz, 1H), 3.87-3.77 (m, 3H), 3.64 (d, J=10.1 Hz, 2H), 3.59 (s, 3H), 3.47 (dd, J=10.3, 5.2 Hz, 4H), 3.16 (dt, J=9.6, 4.9 Hz, 1H), 2.99-2.67 (m, 2H), 2.11 (dd, J=13.3, 3.7 Hz, 1H), 1.59 (d, J=7.0 Hz, 3H), 1.37-1.29 (m, 3H), 1.25 (d, J=6.3 Hz, 3H), 1.03 (t, J=5.7 Hz, 1H). LC/MS (ESI) m/z: 842 (M+H)⁺.

### Scheme 89: Synthesis of (S)-7-(([1,1′-biphenyl]-4-carbonyl)glycyl)-N—((R)-1-(4-carbamimidoylthiophen-2-yl)ethyl)-1,4-dioxa-7-azaspiro[4.4]nonane-8-carboxamide (Compound 90)

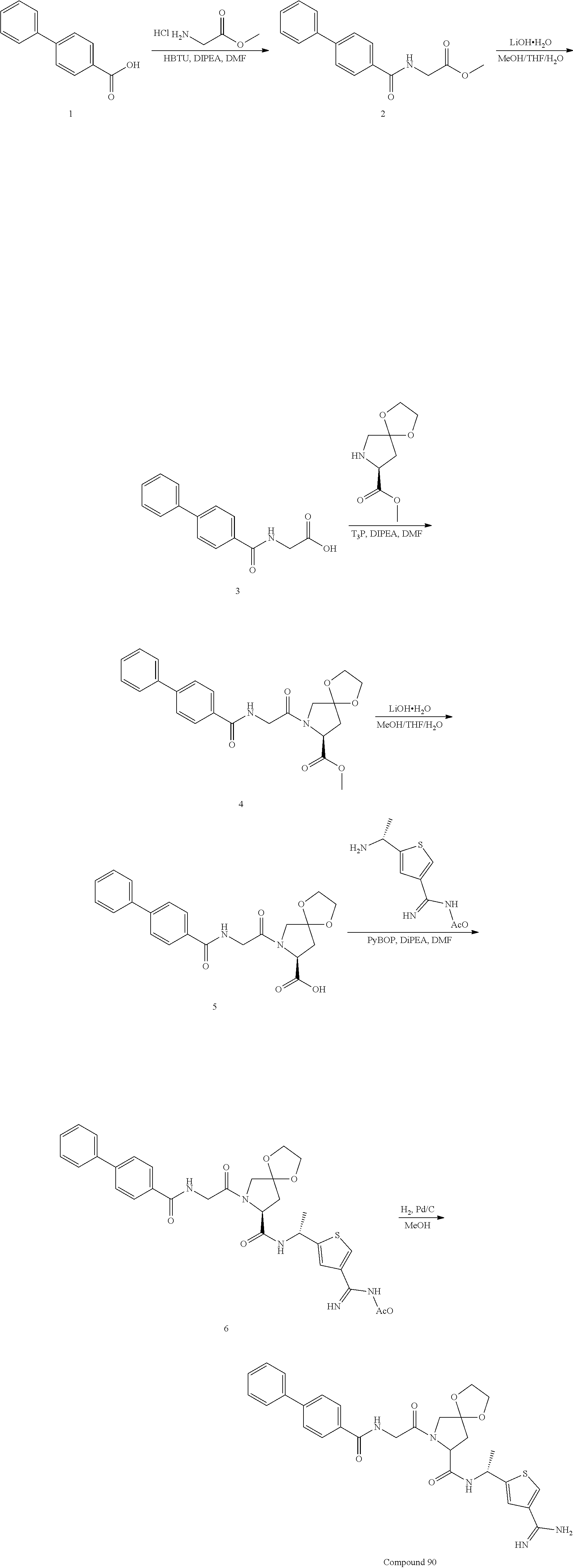

### Step 1: methyl ([1,1′-biphenyl]-4-carbonyl)glycinate (2)

To a mixture of [1,1′-biphenyl]-4-carboxylic acid (500 mg, 2.52 mmol) and methyl glycinate hydrochloride (380 mg, 3.03 mmol) in DMF (5 mL) was added HBTU (1.43 g, 3.78 mmol) and DIPEA (977 mg, 7.56 mmol) under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography on silica gel (PE:EtOAc=5:1) to give methyl ([1,1′-biphenyl]-4-carbonyl)glycinate (320 mg, yield 47.1%) as a white solid. LC/MS (ESI) m/z: 270 (M+H).

### Step 2: ([1,1′-biphenyl]-4-carbonyl)glycine (3)

To a solution of methyl ([1,1′-biphenyl]-4-carbonyl)glycinate (320 mg, 1.19 mmol) in MeOH (3 mL) and water (1 mL) was added LiOH·H₂O (99 mg, 2.38 mmol) at 0° C. and the mixture was stirred at room temperature for 2 hours. The mixture was acidified with 1 N aq·HCl to pH˜3, extracted with EtOAc twice. The combined organic layers were washed with brine, dried over anhydrous Na₂SO₄, filtered, and concentrated under reduced pressure to give ([1,1′-biphenyl]-4-carbonyl)glycine (300 mg, yield 98.7%) as a white solid, which was used directly in the next reaction. LC/MS (ESI) (m/z): 256 (M+H)⁺.

### Step 3: methyl (S)-7-(([1,1′-biphenyl]-4-carbonyl)glycyl)-1,4-dioxa-7-azaspiro[4.4]nonane-8-carboxylate (4)

To a mixture of ([1,1′-biphenyl]-4-carbonyl)glycine (57 mg, 0.22 mmol) and methyl (S)-1,4-dioxa-7-azaspiro[4.4]nonane-8-carboxylate (50 mg, 0.26 mmol) in DMF (1 mL) was added DIPEA (143 mg, 1.11 mmol) and T₃P (211 mg, 0.33 mmol, 50% wt. in EtOAc) at 0° C. under N₂ atmosphere and the mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=20:1) to give methyl (S)-7-(([1,1′-biphenyl]-4-carbonyl)glycyl)-1,4-dioxa-7-azaspiro[4.4]nonane-8-carboxylate (70 mg, yield 75.3%) as a colorless oil. LC/MS (ESI) m/z: 425 (M+H)⁺.

### Step 4: (S)-7-(([1,1′-biphenyl]-4-carbonyl)glycyl)-1,4-dioxa-7-azaspiro[4.4]nonane-6-carboxylic acid (5)

To a solution of methyl (S)-7-(([1,1′-biphenyl]-4-carbonyl)glycyl)-1,4-dioxa-7-azaspiro[4.4]nonane-8-carboxylate (70 mg, 0.165 mmol) in MeOH (1 mL) and water (0.3 mL) was added LiOH·H₂O (14 mg, 0.329 mmol) at 0° C. and the mixture was stirred at room temperature for 2 hours. The mixture was acidified with 1 N aq·HCl to pH-3, extracted with EtOAc twice. The combined organic layers were washed with brine, dried over anhydrous Na₂SO₄, filtered, and concentrated under reduced pressure to give (S)-7-(([1,1′-biphenyl]-4-carbonyl)glycyl)-1,4-dioxa-7-azaspiro[4.4]nonane-8-carboxylic acid (59 mg, yield 86.8%) as a white solid, which was used directly in the next reaction. LC/MS (ESI) (m/z): 411 (M+H)⁺.

### Step 5: (S)-7-(([1,1′-biphenyl]-4-carbonyl)glycyl)-N—((R)-1-(4-(N-acetoxycarbamimidoyl) thiophen-2-yl)ethyl)-1,4-dioxa-7-azaspiro[4.4]nonane-8-carboxamide (6)

To a mixture of (S)-7-(([1,1′-biphenyl]-4-carbonyl)glycyl)-1,4-dioxa-7-azaspiro[4.4]nonane-8-carboxylic acid (59 mg, 0.14 mmol) and (R)—N-acetoxy-5-(1-aminoethyl)thiophene-3-carboximidamide hydrochloride (49 mg, 0.22 mmol) in DMF (2 mL) was added DIPEA (93 mg, 0.72 mmol) and PyBOP (137 mg, 0.22 mmol) at 0° C. under N₂ atmosphere and the mixture was stirred at 25° C. for 2 hours. The mixture was diluted with EtOAc, washed with water and brine, dried over anhydrous Na₂SO₄, filtered, and concentrated under reduced pressure to dryness. The residue was purified by prep-TLC (DCM:MeOH=10:1) to give (S)-7-(([1,1′-biphenyl]-4-carbonyl)glycyl)-N—((R)-1-(4-(N-acetoxycarbamimidoyl)thiophen-2-yl)ethyl)-1,4-dioxa-7-azaspiro[4.4]nonane-8-carboxamide (34 mg, yield 39.5%) as a white solid. LC/MS (ESI) m/z: 620 (M+H)⁺.

### Step 6: (S)-7-(([1,1′-biphenyl]-4-carbonyl)glycyl)-N—((R)-1-(4-carbamimidoylthiophen-2-yl)ethyl)-1,4-dioxa-7-azaspiro[4.4]nonane-6-carboxamide (Compound 90)

To a mixture of (S)-7-(([1,1′-biphenyl]-4-carbonyl)glycyl)-N—((R)-1-(4-(N-acetoxycarbamimidoyl) thiophen-2-yl)ethyl)-1,4-dioxa-7-azaspiro[4.4]nonane-8-carboxamide (34 mg, 0.055 mmol) in MeOH (3 mL) was added Pd/C (4 mg, 10% wt.), the mixture was degassed under N₂ atmosphere for three times and stirred under a H₂ balloon at 25° C. for 2 hours. The mixture was filtered, and the filtrate was concentrated under reduced pressure to dryness. The residue was purified by prep-HPLC to give Compound 90 (2.2 mg, yield 7.1%) as a white solid. ¹H NMR (400 MHz, CD₃OD) δ 8.54 (s, 1H), 8.25 (d, J=9.5 Hz, 1H), 7.94 (d, J=8.4 Hz, 2H), 7.74 (d, J=8.3 Hz, 2H), 7.67 (d, J=7.3 Hz, 2H), 7.54 (d, J=7.5 Hz, 1H), 7.48 (t, J=7.5 Hz, 2H), 7.39 (t, J=7.4 Hz, 1H), 5.33-5.25 (m, 1H), 4.58 (t, J=7.6 Hz, 1H), 4.17 (d, J=16.4 Hz, 2H), 4.02-3.95 (m, 4H), 3.80 (q, J=10.9 Hz, 2H), 2.44 (dd, J=12.9, 9.1 Hz, 1H), 2.21 (dd, J=13.0, 6.5 Hz, 1H), 1.63 (dd, J=36.8, 6.8 Hz, 3H). LC/MS (ESI) m/z: 562 (M+H)⁺.

### Scheme 90: Synthesis of (S)-7-((9,9-difluoro-9H-fluorene-3-carbonyl)glycyl)-N—((R)-1-(4-(pyrimidin-5-yl)thiophen-2-yl)ethyl)-1,4-dioxa-7-azaspiro[4.4]nonane-8-carboxamide (Compound 91)

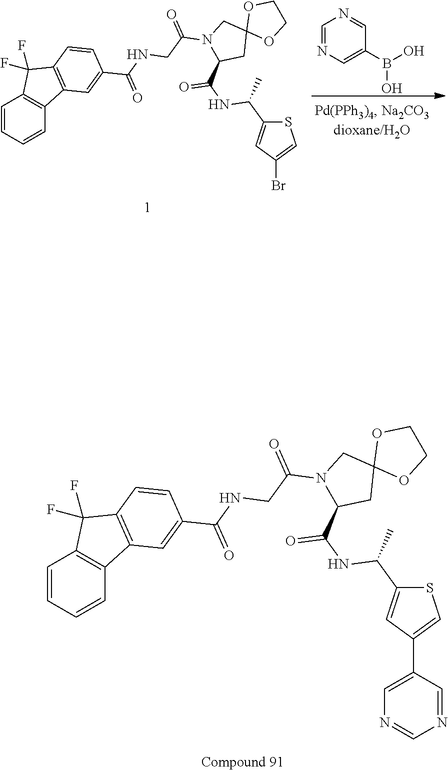 [SMILES: C[C@@H](NC(=O)[C@@H]1CC2(CN1C(=O)CNC(=O)c1ccc3c(c1)-c1ccccc1C3(F)F)OCCO2)c1cc(Br)cs1 ; OB(O)c1cncnc1 ; C[C@@H](NC(=O)[C@@H]1CC2(CN1C(=O)CNC(=O)c1ccc3c(c1)-c1ccccc1C3(F)F)OCCO2)c1cc(-c2cncnc2)cs1]

To a solution of (S)—N—((R)-1-(4-bromothiophen-2-yl)ethyl)-7-((9,9-difluoro-9H-fluorene-3-carbonyl)glycyl)-1,4-dioxa-7-azaspiro[4.4]nonane-8-carboxamide (40 mg, 0.062 mmol) in 1,4-dioxane (1 mL) and water (0.3 mL) was added pyrimidin-5-ylboronic acid (15 mg, 0.124 mmol), NaHCO₃ (197 mg, 1.86 mmol) and Pd(PPh₃)₄ (7 mg, 0.0062 mmol) at room temperature. The mixture was degassed under N₂ atmosphere for three times and stirred under a N₂ balloon at 90° C. overnight. The mixture was diluted with EtOAc, washed with water and brine, dried over anhydrous Na₂SO₄, filtered, and concentrated under reduced pressure to dryness. The residue was purified by prep-HPLC to give Compound 91 (4.4 mg, yield 11.0%) as a white solid. ¹H NMR (400 MHz, CD₃OD) δ 9.05 (d, J=4.1 Hz, 2H), 8.85 (s, 1H), 8.11 (s, 1H), 7.85-7.82 (m, 1H), 7.78 (s, 1H), 7.66 (dd, J=12.5, 8.8 Hz, 4H), 7.53 (d, J=7.3 Hz, 1H), 7.43 (t, J=7.5 Hz, 1H), 5.32 (q, J=6.6 Hz, 1H), 4.66-4.59 (m, 1H), 4.21 (dd, J=38.2, 16.6 Hz, 2H), 4.00 (dt, J=12.7, 5.6 Hz, 4H), 3.80 (d, J=11.9 Hz, 2H), 2.44 (dd, J=13.0, 8.9 Hz, 1H), 2.25 (dd, J=13.1, 6.8 Hz, 1H), 1.63 (dd, J=34.7, 7.0 Hz, 3H). LC/MS (ESI) m/z: 646 (M+H)⁺.

### Scheme 91: Synthesis of (S)—N—((R)-1-(4-(2H-tetrazol-5-yl)thiophen-2-yl)ethyl)-7-((9,9-difluoro-9H-fluorene-3-carbonyl)glycyl)-1,4-dioxa-7-azaspiro[4.4]nonane-8-carboxamide (Compound 92)

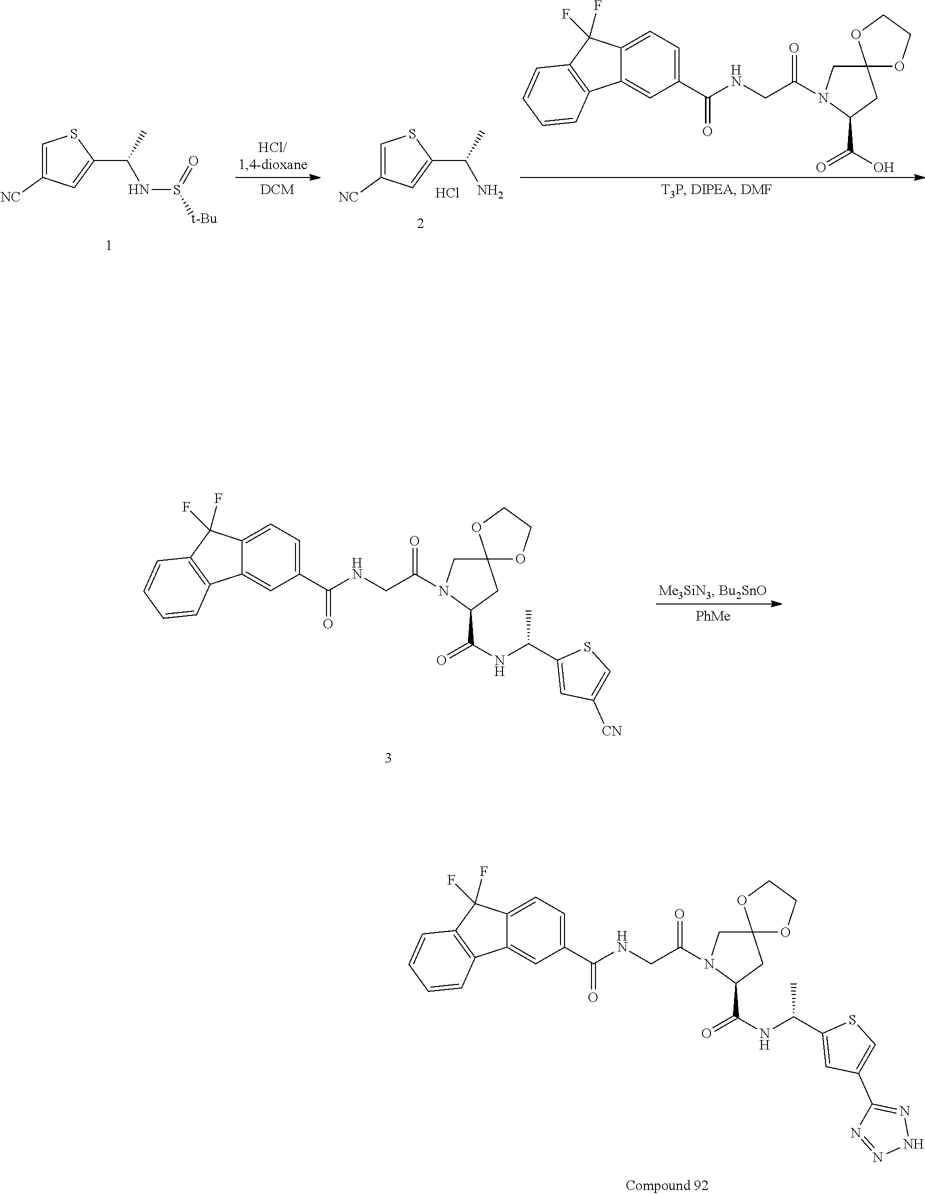

### Step 1: (S)-5-(1-aminoethyl)thiophene-3-carbonitrile hydrochloride (2)

A solution of 1-(tert-butyl) 2-methyl (2S,4S)-4-phenylpyrrolidine-1,2-dicarboxylate (100 mg, 0.39 mmol) in HCl/1,4-dioxane (3 mL) was stirred at room temperature for 2 hours. The mixture was concentrated to dryness under reduced pressure to give (S)-5-(1-aminoethyl)thiophene-3-carbonitrile hydrochloride (80 mg, crude) as a white solid, which was directly used in the next reaction without further purification. LC/MS (ESI) m/z: 153 (M+H)⁺.

### Step 2: (S)—N—((R)-1-(4-cyanothiophen-2-yl)ethyl)-7-((9,9-difluoro-9H-fluorene-3-carbonyl) glycyl)-1,4-dioxa-7-azaspiro[4.4]nonane-8-carboxamide (3)

To a mixture of (S)-7-((9,9-difluoro-9H-fluorene-3-carbonyl)glycyl)-1,4-dioxa-7-azaspiro[4.4]nonane-8-carboxylic acid (30 mg, 0.066 mmol) and (S)-5-(1-aminoethyl)thiophene-3-carbonitrile hydrochloride (25 mg, 0.131 mmol) in DMF (1 mL) was added DIPEA (43 mg, 0.33 mmol) and T₃P (63 mg, 0.099 mmol, 50% wt. in EtOAc) at 0° C. under N₂ atmosphere and the mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=20:1) to give (S)—N—((R)-1-(4-cyanothiophen-2-yl)ethyl)-7-((9,9-difluoro-9H-fluorene-3-carbonyl)glycyl)-1,4-dioxa-7-azaspiro[4.4]nonane-8-carboxamide (30 mg, yield 76.9%) as a colorless oil. LC/MS (ESI) m/z: 593 (M+H)⁺.

### Step 3: (S)—N—((R)-1-(4-(2H-tetrazol-5-yl)thiophen-2-yl)ethyl)-7-((9,9-difluoro-9H-fluorene-3-carbonyl)glycyl)-1,4-dioxa-7-azaspiro[4.4]nonane-8-carboxamide (Compound 92)

To a solution of (S)—N—((R)-1-(4-cyanothiophen-2-yl)ethyl)-7-((9,9-difluoro-9H-fluorene-3-carbonyl)glycyl)-1,4-dioxa-7-azaspiro[4.4]nonane-8-carboxamide (25 mg, 0.042 mmol) in toluene (2 mL) was added MeSiN₃ (15 mg, 0.13 mmol) and Bu₂SnO (31 mg, 0.13 mmol) under N₂ atmosphere and the mixture was stirred under N₂ atmosphere at 130° C. for 4 hours. The mixture was concentrated to dryness under reduced pressure and the residue was purified by prep-HPLC to give (S)—N—((R)-1-(4-(2H-tetrazol-5-yl)thiophen-2-yl)ethyl)-7-((9,9-difluoro-9H-fluorene-3-carbonyl) glycyl)-1,4-dioxa-7-azaspiro[4.4]nonane-8-carboxamide (1.4 mg, yield 46.2%) as a white solid. ¹H NMR (400 MHz, CD₃OD) δ 8.16 (d, J=9.7 Hz, 1H), 8.00 (d, J=7.4 Hz, 1H), 7.87 (d, J=7.8 Hz, 1H), 7.71 (dd, J=19.1, 7.6 Hz, 2H), 7.65-7.61 (m, 2H), 7.56 (t, J=7.5 Hz, 1H), 7.43 (t, J=7.5 Hz, 1H), 5.34 (q, J=6.5 Hz, 1H), 4.61 (dd, J=8.9, 6.0 Hz, 1H), 4.21 (q, J=16.7 Hz, 2H), 3.99 (d, J=16.5 Hz, 4H), 3.80 (d, J=9.2 Hz, 2H), 2.45 (dd, J=13.1, 9.2 Hz, 1H), 2.24 (dd, J=13.2, 6.1 Hz, 1H), 1.64 (dd, J=35.8, 7.0 Hz, 3H). LC/MS (ESI) m/z: 636 (M+H)⁺.

### Scheme 92: (2S,4R)—N—((R)-1-(4-carbamimidoylthiophen-2-yl)ethyl)-4-cyclohexyl-1-((4-(4-fluorophenoxy)benzoyl)glycyl)pyrrolidine-2-carboxamide(Compound 93)

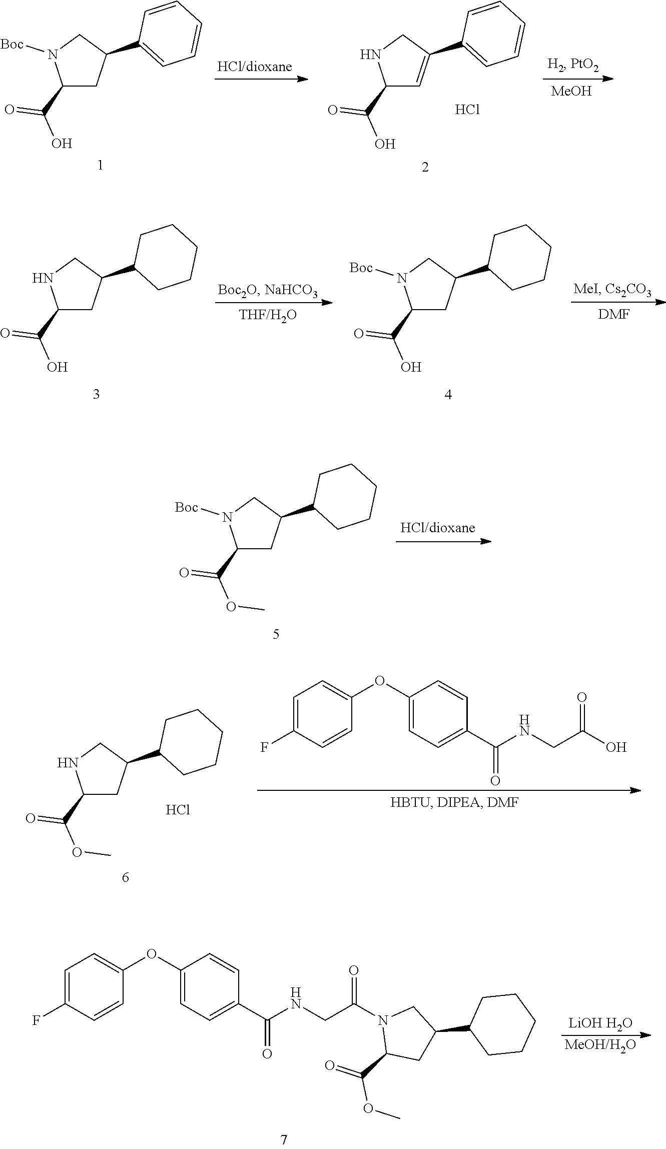

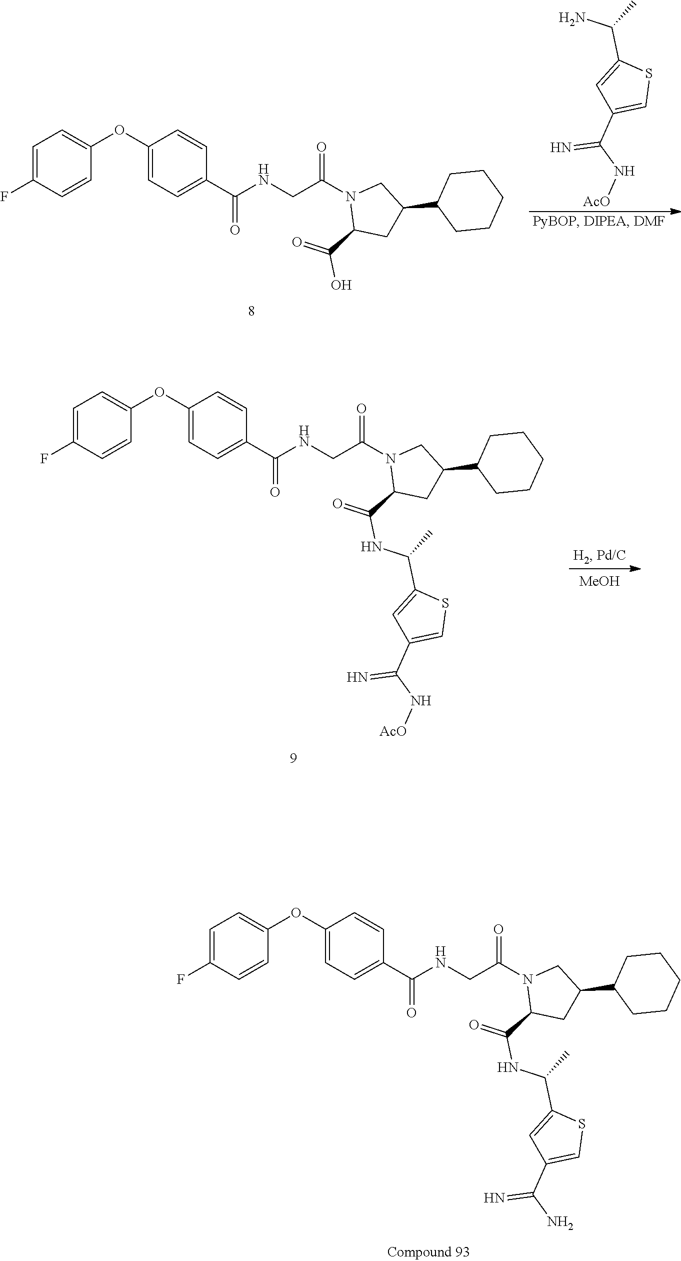

### Step 1: (2S,4R)-4-phenylpyrrolidine-2-carboxylic acid hydrochloride (2)

To a solution of (2S,4R)-1-(tert-butoxycarbonyl)-4-phenylpyrrolidine-2-carboxylic acid (1.0 g, 3.4 mmol) in HCl/1,4-dioxane (12 mL) was stirred under N₂ atmosphere at room temperature for 2 hours. The reaction mixture was concentrated to dryness under reduced pressure, diluted with DCM again and dried under vacuum to give (2S,4R)-4-phenylpyrrolidine-2-carboxylic acid hydrochloride (650 mg, yield 84.1%), which was used directly in the next step without further purification. LC/MS (ESI) m/z: 192 (M+H)⁺.

### Step 2: (2S,4R)-4-cyclohexylpyrrolidine-2-carboxylic acid (3)

To a solution of (2S,4R)-4-phenylpyrrolidine-2-carboxylic acid hydrochloride (650 mg, 3.4 mmol) in MeOH (10 mL) was added PtO₂ (80 mg, 0.34 mmol) at room temperature and the mixture was stirred under H₂ atmosphere at room temperature overnight. The mixture was filtered, and the filtrate was concentrated to dryness under reduced pressure to give (2S,4R)-4-cyclohexylpyrrolidine-2-carboxylic acid (500 mg, yield 74.4%) as a colorless oil, which was used directly in the next step. LC/MS (ESI) (m/z): 198 (M+H)⁺.

### Step 3: (2S,4R)-1-(tert-butoxycarbonyl)-4-cyclohexylpyrrolidine-2-carboxylic acid (4)

To a solution of (2S,4R)-4-cyclohexylpyrrolidine-2-carboxylic acid (500 mg, 2.5 mmol) in THF (8 mL) and water (8 mL) was added NaHCO₃ (1.0 g, 12.5 mmol) and Boc₂O (1.6 g, 7.6 mmol) under N₂ atmosphere at room temperature and stirred overnight. The mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (PE:EtOAc=1:1) to give (2S,4R)-1-(tert-butoxycarbonyl)-4-cyclohexylpyrrolidine-2-carboxylic acid (580 mg, yield 78.0%) as a colorless oil. LCMS (ESI) m/z=298 (M+H)⁺.

### Step 4: 1-(tert-butyl) 2-methyl (2S,4R)-4-cyclohexylpyrrolidine-1,2-dicarboxylate (5)

To a solution of (2S,4R)-1-(tert-butoxycarbonyl)-4-cyclohexylpyrrolidine-2-carboxylic acid (580 mg, 1.9 mmol) in DMF (10 mL) was added CS₂CO₃ (3.7 g, 11 mmol) and iodomethane (1.6 g, 11 mmol) under N₂ atmosphere at room temperature and the reaction mixture was stirred overnight. The mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (PE:EtOAc=2:1) to give 1-(tert-butyl) 2-methyl (2S,4R)-4-cyclohexylpyrrolidine-1,2-dicarboxylate (530 mg, yield 89.5%) as a colorless oil. LCMS (ESI) m/z=312 (M+H)⁺.

### Step 5: methyl (2S,4R)-4-cyclohexylpyrrolidine-2-carboxylate hydrochloride (6)

A solution of 1-(tert-butyl) 2-methyl (2S,4R)-4-cyclohexylpyrrolidine-1,2-dicarboxylate (120 mg, 0.39 mmol) in HCl/1,4-dioxane (2 mL) was stirred under N₂ atmosphere at room temperature for 2 hours. The reaction mixture was concentrated to dryness under reduced pressure to give methyl (2S,4R)-4-cyclohexylpyrrolidine-2-carboxylate hydrochloride (80 mg, yield 83.3%), which was used directly in the next step without further purification. LC/MS (ESI) m/z: 212 (M+H)⁺.

### Step 6: methyl (2S,4R)-4-cyclohexyl-1-((4-(4-fluorophenoxy)benzoyl)glycyl)pyrrolidine-2-carboxylate (7)

To a mixture of methyl (2S,4R)-4-cyclohexylpyrrolidine-2-carboxylate hydrochloride (40 mg, 0.19 mmol) and (4-(4-fluorophenoxy)benzoyl)glycine (55 mg, 0.19 mmol) in DMF (2 mL) was added DIPEA (122 mg, 0.95 mmol) and HBTU (95 mg, 0.25 mmol) at 0° C. under N₂ atmosphere and the mixture was stirred at 25° C. for 2 hours. The mixture was diluted with water, extracted with EtOAc twice. The combined organic layers were dried over anhydrous Na₂SO₄, filtered, and concentrated to dryness under reduced pressure. The residue was purified by column chromatography on silica gel (PE:EtOAc=2:1) to give methyl (2S,4R)-4-cyclohexyl-1-((4-(4-fluorophenoxy)benzoyl)glycyl)pyrrolidine-2-carboxylate (80 mg, yield 87.3%) as a colorless oil. LC/MS (ESI) m/z: 493 (M+H)⁺.

### Step 7: (2S,4R)-4-cyclohexyl-1-((4-(4-fluorophenoxy)benzoyl)glycyl)pyrrolidine-2-carboxylic acid (8)

To a solution of methyl (2S,4R)-4-cyclohexyl-1-((4-(4-fluorophenoxy)benzoyl)glycyl) pyrrolidine-2-carboxylate (80 mg, 0.17 mmol) in MeOH (2 mL) and water (1 mL) was added LiOH·H₂O (43 mg, 1.1 mmol) at 0° C. and the mixture was stirred at room temperature for 2 hours. The mixture was acidified with 1 N aq·HCl to pH˜3, extracted with EtOAc twice. The combined organic layers were washed with brine, dried over anhydrous Na₂SO₄, filtered, and concentrated to dryness under reduced pressure to give (2S,4R)-4-cyclohexyl-1-((4-(4-fluorophenoxy)benzoyl)glycyl)pyrrolidine-2-carboxylic acid (75 mg, yield 94.1%) as a colorless oil, which was used directly in the next step. LC/MS (ESI) (m/z): 469 (M+H)⁺.

### Step 8: (2S,4R)—N—((R)-1-(4-(N-acetoxycarbamimidoyl)thiophen-2-yl)ethyl)-4-cyclohexyl-1-((4-(4-fluorophenoxy)benzoyl)glycyl)pyrrolidine-2-carboxamide (9)

To a mixture of (2S,4R)-4-cyclohexyl-1-((4-(4-fluorophenoxy)benzoyl)glycyl)pyrrolidine-2-carboxylic acid (40 mg, 0.085 mmol) and (R)—N-acetoxy-5-(1-aminoethyl)thiophene-3-carboximidamide (30 mg, 0.13 mmol) in DMF (2 mL) was added DIPEA (55 mg, 0.42 mmol) and PyBOP (57 mg, 0.11 mmol) at 0° C. under N₂ atmosphere and the mixture was stirred at 25° C. for 2 hours. The mixture was diluted with water, extracted with EtOAc twice. The combined organic layers were dried over anhydrous Na₂SO₄, filtered, and concentrated to dryness under reduced pressure. The residue was purified by column chromatography on silica gel (DCM:MeOH=12:1) to give (2S,4R)—N—((R)-1-(4-(N-acetoxycarbamimidoyl)thiophen-2-yl)ethyl)-4-cyclohexyl-1-((4-(4-fluorophenoxy)benzoyl)glycyl)pyrrolidine-2-carboxamide (30 mg, yield 52.1%) as a colorless oil. LC/MS (ESI) m/z: 678 (M+H)⁺.

### Step 9: (2S,4R)—N—((R)-1-(4-carbamimidoylthiophen-2-yl)ethyl)-4-cyclohexyl-1-((4-(4-fluorophenoxy)benzoyl)glycyl)pyrrolidine-2-carboxamide (Compound 93)

To a solution of (2S,4R)—N—((R)-1-(4-(N-acetoxycarbamimidoyl)thiophen-2-yl)ethyl)-4-cyclohexyl-1-((4-(4-fluorophenoxy)benzoyl)glycyl)pyrrolidine-2-carboxamide (30 mg, 0.044 mmol) in MeOH (2 mL) was added Pd/C (10 mg, 10% wt.) at room temperature and the mixture was stirred under H₂ atmosphere at 25° C. overnight. The mixture was filtered and purified by prep-HPLC to give Compound 93 (4 mg, yield 14.7%) as a white solid. ¹H NMR (400 MHz, CD₃OD) δ 8.50 (s, 1H), 8.23 (t, J=3.7 Hz, 1H), 7.86 (d, J=8.7 Hz, 2H), 7.49 (d, J=15.3 Hz, 1H), 7.16 (t, J=8.7 Hz, 2H), 7.08 (dd, J=9.1, 4.5 Hz, 2H), 6.99 (d, J=8.7 Hz, 2H), 5.28 (dt, J=20.6, 6.9 Hz, 1H), 4.54 (d, J=9.0 Hz, 1H), 4.16 (t, J=10.7 Hz, 2H), 3.94 (t, J=8.4 Hz, 1H), 2.15 (dd, J=18.1, 6.9 Hz, 2H), 1.93-1.86 (m, 1H), 1.71 (dd, J=26.2, 13.1 Hz, 6H), 1.57 (d, J=7.0 Hz, 3H), 1.27 (d, J=12.5 Hz, 4H), 1.02 (dd, J=22.0, 9.7 Hz, 2H). LC/MS (ESI) (m/z): 620 (M+H)⁺.

### Scheme 93: The synthesis of (S)—N—((R)-1-(4-carbamimidoylthiophen-2-yl)ethyl)-7-((9,9-dimethyl-9H-fluorene-3-carbonyl)glycyl)-1,4-dioxa-7-azaspiro[4.4]nonane-8-carboxamide (Compound 94)

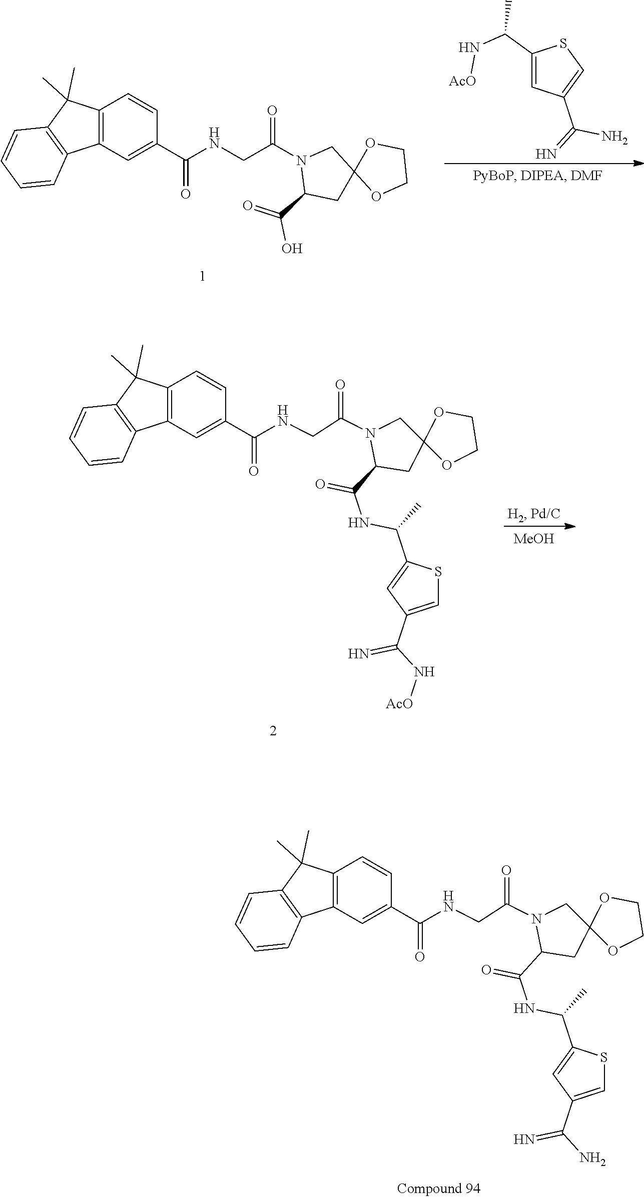

Compound 94 was prepared as a white solid from (S)-7-((9,9-dimethyl-9H-fluorene-3-carbonyl)glycyl)-1,4-dioxa-7-azaspiro[4.4]nonane-8-carboxylic acid and (R)-5-(1-(acetoxyamino)ethyl)thiophene-3-carboximidamide based on the procedures set forth in Scheme 92. ¹H NMR (400 MHz, CD₃OD) δ 8.54 (s, 1H), 8.26-8.20 (m, 2H), 7.80 (ddd, J=12.8, 6.6, 1.7 Hz, 2H), 7.57 (d, J=7.9 Hz, 1H), 7.53-7.47 (m, 2H), 7.38-7.34 (m, 2H), 5.38-5.26 (m, 1H), 4.58 (d, J=1.8 Hz, 1H), 4.22-4.12 (m, 2H), 4.03-3.95 (m, 4H), 3.85-3.76 (m, 2H), 2.43 (dt, J=21.3, 10.6 Hz, 1H), 2.21 (dd, J=13.1, 6.5 Hz, 1H), 1.62 (dd, J=29.8, 7.2 Hz, 3H), 1.49 (s, 6H). LC/MS (ESI) (m/z): 602 (M+H)⁺.

### Scheme 94: Synthesis of (S)-7-((biphenylene-2-carbonyl)glycyl)-N—((R)-1-(4-carbamimidoylthiophen-2-yl)ethyl)-1,4-dioxa-7-azaspiro[4.4]nonane-8-carboxamide (Compound 95)

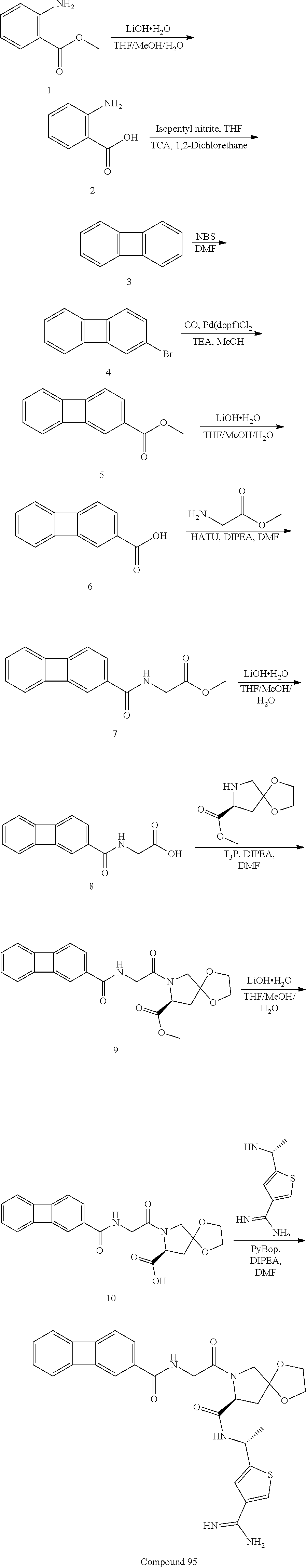

### Step 1: 2-aminobenzoic acid (2)

To a solution of methyl 2-aminobenzoate (10 g, 66.15 mmol) in THF/MeOH/H₂O (60 mL,4/1/1) was added LiOH (5.6 g, 132.31 mmol) under N₂ atmosphere and the reaction mixture was stirred at room temperature overnight. The mixture was acidified with 1 N aq·HCl to pH˜3, extracted with EtOAc twice. The combined organic layers were washed with brine, dried over anhydrous Na₂SO₄, filtered, and concentrated under reduced pressure to dryness. The residue was purified by flash chromatography on silica gel (DCM:MeOH=20:1) to give 2-aminobenzoic acid (8.5 g, yield 93.7%) as a white solid. LC/MS (ESI) m/z:138 (M+H)⁺.

### Step 2: biphenylene (3)

To a solution of 2-aminobenzoic acid (9.5 g, 69.27 mmol) in THF (100 mL) was added isopentyl nitrite (10.1 g, 86.59 mmol). After stirred at room temperature for 1 hour, TCA (100 mg) was added into the above mixture. After stirred at room temperature for 1 hour, the mixture was filtered, and the solid was washed with 1,2-dichloroethane (25 mL) for three times. Then the resulting solid was suspended in 1,2-dichloroethane (75 mL). The suspension was added into the gently boiling 1,2-dichloroethane (100 mL) and the reaction mixture was stirred at 85° C. for 1 hour. The mixture was diluted with EtOAc and washed with water and brine, dried over anhydrous Na₂SO₄, filtered, and concentrated under reduced pressure to dryness. The residue was purified by flash chromatography on silica gel (PE:EtOAc=50:1) to give biphenylene (1.6 g, yield 15.2%) as a white solid. LC/MS (ESI) m/z:153 (M+H)⁺.

### Step 3: 2-bromobiphenylene (4)

To a solution of biphenylene (1.6 g, 10.5 mmol) in DMF (20 mL) was added NBS (2.1 g, 11.6 mmol) under N₂ atmosphere and the reaction mixture was stirred at room temperature overnight. The mixture was diluted with EtOAc and washed with water and brine, dried over anhydrous Na₂SO₄, filtered, and concentrated under reduced pressure to dryness. The residue was purified by flash chromatography on silica gel (PE:EtOAc=50:1) to give 2-bromobiphenylene (2.15 g, yield 88.5%) as a white solid. LC/MS (ESI) m/z:231 (M+H)⁺.

### Step 4: methyl biphenylene-2-carboxylate (5)

To a solution of 2-bromobiphenylene (1 g, 4.33 mmol) in MeOH (30 mL) was added TEA (1.3 g, 12.98 mmol) and Pd(dppf)Cl₂ (158 mg, 0.22 mmol) and the reaction mixture was stirred under a CO balloon at 80° C. for 2 days. The mixture was diluted with EtOAc and washed with water and brine, dried over anhydrous Na₂SO₄, filtered, and concentrated under reduced pressure to dryness. The residue was purified by flash chromatography on silica gel (PE:EtOAc=5:1) to give methyl biphenylene-2-carboxylate (450 mg, yield 49.5%) as a white solid. LC/MS (ESI) m/z:211 (M+H)⁺.

### Step 5: biphenylene-2-carboxylic acid (6)

To a solution of methyl biphenylene-2-carboxylate (450 mg, 2.14 mmol) in THF/MeOH/H₂O (6 mL,4/1/1) was added LIOH (180 mg, 4.28 mmol) under N₂ atmosphere and the reaction mixture was stirred at room temperature for 3 hours. The mixture was acidified with 1 N aq·HCl to pH˜3, extracted with EtOAc twice. The combined organic layers were washed with brine, dried over anhydrous Na₂SO₄, filtered, and concentrated under reduced pressure to dryness. The residue was purified by flash chromatography on silica gel (DCM:MeOH=20:1) to give biphenylene-2-carboxylic acid (360 mg, yield 85.7%) as a white solid. LC/MS (ESI) m/z:197 (M+H)⁺.

### Step 6: methyl (biphenylene-2-carbonyl)glycinate (7)

To a mixture of biphenylene-2-carboxylic acid (360 mg, 1.83 mmol) and methyl glycinate (245 mg, 2.75 mmol) in DMF (10 mL) was added HATU (1.0 g, 2.75 mmol) and DIPEA (711 mg, 5.50 mmol) under N₂ atmosphere and the reaction mixture was stirred at room temperature overnight. The mixture was diluted with EtOAc and washed with water and brine, dried over anhydrous Na₂SO₄, filtered, and concentrated under reduced pressure to dryness. The residue was purified by flash chromatography on silica gel (PE:EtOAc=5:1) to give methyl (biphenylene-2-carbonyl)glycinate (460 mg, yield 93.8%) as a white solid.LC/MS (ESI) m/z:268 (M+H)⁺.

### Step 7: (biphenylene-2-carbonyl)glycine (8)

To a solution of methyl (biphenylene-2-carbonyl)glycinate (460 mg, 1.72 mmol) in THF/MeOH/H₂O (6 mL,4/1/1) was added UGH (145 mg, 3.44 mmol) under N₂ atmosphere and the reaction mixture was stirred at room temperature for 3 hours. The mixture was acidified with 1 N aq·HCl to pH˜3, extracted with EtOAc twice. The combined organic layers were washed with brine, dried over anhydrous Na₂SO₄, filtered, and concentrated under reduced pressure to dryness. The residue was purified by flash chromatography on silica gel (DCM:MeOH=20:1) to give (biphenylene-2-carbonyl)glycine (410 mg, yield 94.1%) as a white solid. LC/MS (ESI) m/z:254 (M+H)⁺.

### Step 6: methyl (S)-7-((biphenylene-2-carbonyl)glycyl)-1,4-dioxa-7-azaspiro[4.4]nonane-6-carboxylate (9)

To a mixture of (biphenylene-2-carbonyl)glycine (97 mg, 0.39 mmol) and methyl (S)-1,4-dioxa-7-azaspiro[4.4]nonane-8-carboxylate (72 mg, 0.39 mmol) in DMF (3 mL) was added DIPEA (298 mg, 2.31 mmol) and T₃P (367 mg, 0.58 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography on silica gel (PE:EtOAc=3:1) to give methyl (S)-7-((biphenylene-2-carbonyl)glycyl)-1,4-dioxa-7-azaspiro[4.4]nonane-8-carboxylate (130 mg, yield 80.0%) as a white solid. LC/MS (ESI) m/z: 423 (M+H)⁺.

### Step 9: (S)-7-((biphenylene-2-carbonyl)glycyl)-1,4-dioxa-7-azaspiro[4.4]nonane-8-carboxylic acid (10)

To a solution of methyl (S)-7-((biphenylene-2-carbonyl)glycyl)-1,4-dioxa-7-azaspiro[4.4]nonane-8-carboxylate (130 mg, 0.31 mmol) in THF/MeOH/H₂O (3 mL,4/1/1) was added LiOH (26 mg, 0.62 mmol) under N₂ atmosphere and the reaction mixture was stirred at room temperature for 3 hours. The mixture was acidified with 1 N aq·HCl to pH-3, extracted with EtOAc twice. The combined organic layers were washed with brine, dried over anhydrous Na₂SO₄, filtered, and concentrated under reduced pressure to dryness. The residue was purified by flash chromatography on silica gel (DCM:MeOH=20:1) to give (S)-7-((biphenylene-2-carbonyl)glycyl)-1,4-dioxa-7-azaspiro[4.4]nonane-8-carboxylic acid (125 mg, yield 99.5%) as a white solid. LC/MS (ESI) m/z:409 (M+H)⁺.

### Step 10: (S)-7-((biphenylene-2-carbonyl)glycyl)-N—((R)-1-(4-carbamimidoylthiophen-2-yl)ethyl)-1,4-dioxa-7-azaspiro[4.4]nonane-8-carboxamide (Compound 95)

To a mixture of (S)-7-((biphenylene-2-carbonyl)glycyl)-1,4-dioxa-7-azaspiro[4.4]nonane-8-carboxylic acid (125 mg, 0.31 mmol) and (R)-5-(1-aminoethyl)thiophene-3-carboximidamide (104 mg, 0.61 mmol) in DMF (5 mL) was added DIPEA (237 mg, 1.84 mmol) and PyBop (239 mg, 0.46 mmol) under N₂ atmosphere and the reaction mixture was stirred at room temperature for 2 hours. The mixture was diluted with EtOAc and washed with water and brine, dried over anhydrous Na₂SO₄, filtered, and concentrated under reduced pressure to dryness. The residue was purified by prep-TLC (DCM:MeOH=5:1) and further purified by prep-HPLC to give Compound 95 (5 mg, yield 2.9%) as a white solid. ¹H NMR (400 MHz, DMSO) δ 8.85-8.53 (m, 1H), 8.50-8.31 (m, 3H), 7.55 (d, J=10.2 Hz, 1H), 7.37 (d, J=7.4 Hz, 1H), 7.15 (s, 1H), 6.88-6.81 (m, 5H), 5.16 (dd, J=23.9, 16.6 Hz, 1H), 4.65-4.35 (m, 1H), 4.08 (dd, J=16.4, 5.3 Hz, 1H), 3.93 (dd, J=19.3, 9.2 Hz, 5H), 3.77 (d, J=10.9 Hz, 1H), 3.61 (d, J=10.7 Hz, 1H), 2.36-2.28 (m, 1H), 2.13 (ddd, J=20.6, 8.8, 5.6 Hz, 1H), 1.55-1.42 (m, 3H).LC/MS (ESI) m/z:560 (M+H)⁺.

### Scheme 95: Synthesis of (S)—N—((R)-1-(4-(1H-imidazol-4-yl)thiophen-2-yl)ethyl)-7-((9,9-difluoro-9H-fluorene-3-carbonyl)glycyl)-1,4-dioxa-7-azaspiro[4.4]nonane-8-carboxamide (Compound 96)

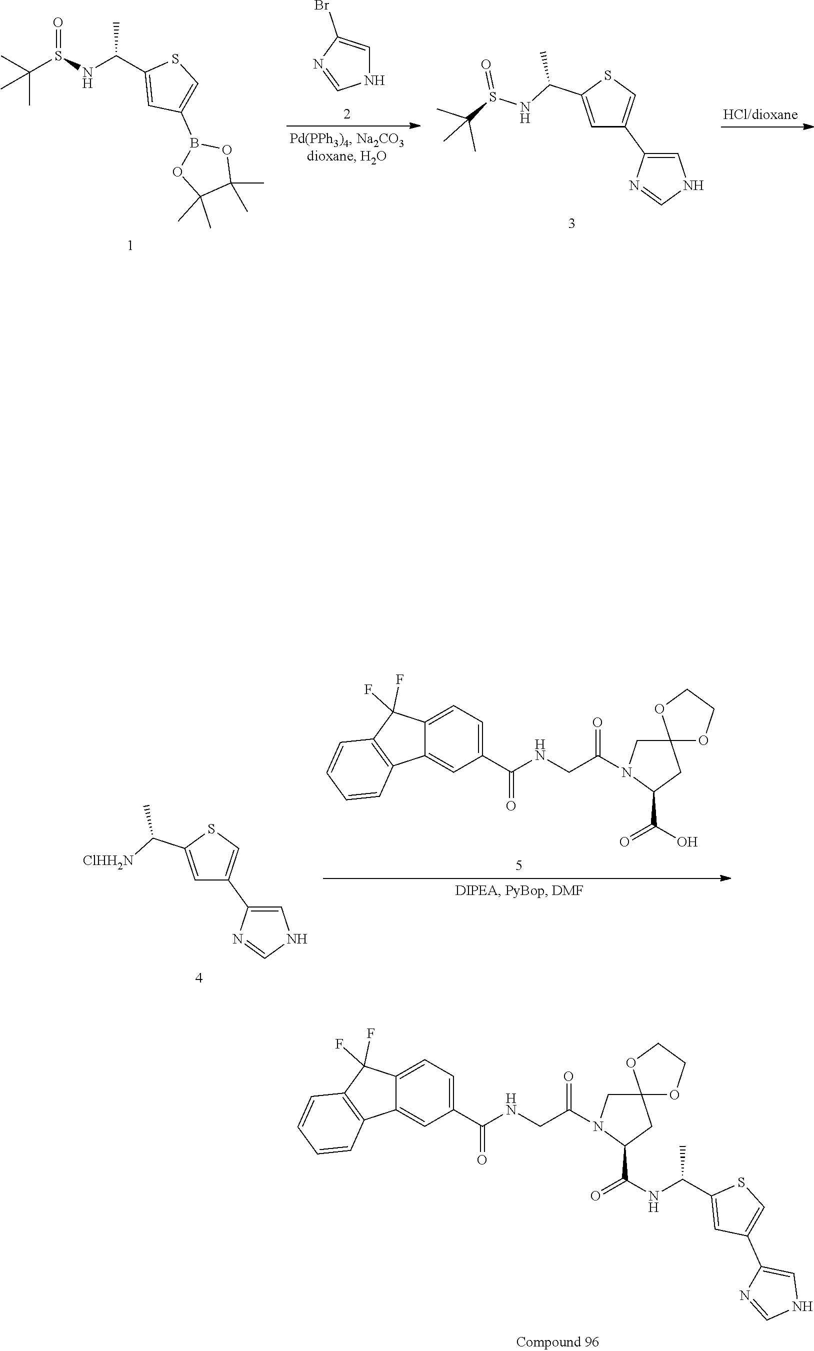

### Step 1: (R)—N—((R)-1-(4-(1H-imidazol-4-yl)thiophen-2-yl)ethyl)-2-methylpropane-2-sulfinamide (3)

To a solution of (R)-2-methyl-N—((R)-1-(4-(4,4,5,5-tetramethyl-1,3,2-dioxaborolan-2-yl)thiophen-2-yl)ethyl)propane-2-sulfinamide (100 mg, 0.28 mmol) in dioxane/H₂O (5 mL, 4/1) was added 4-bromo-1H-imidazole (83 mg, 0.56 mmol), Na₂CO₃ (89 mg, 0.84 mmol) and Pd(PPh₃)₄ (13 mg, 0.04 mmol) under N₂ atmosphere and the reaction mixture was stirred at 90° C. overnight. The mixture was extracted with ethyl acetate and the combined organic layers were washed with brine, dried over anhydrous Na₂SO₄, filtered, and concentrated under reduced pressure to dryness. The residue was purified by flash chromatography on silica gel (PE:EtOAc=2:1) to afford (R)—N—((R)-1-(4-(1H-imidazol-4-yl)thiophen-2-yl)ethyl)-2-methylpropane-2-sulfinamide (81 mg, yield 97.3%) as a yellow solid. LC/MS (ESI) m/z: 298 (M+H)⁺.

### Step 2: (R)-1-(4-(1H-imidazol-4-yl)thiophen-2-yl)ethan-1-amine hydrochloride (4)

To a solution of (R)—N—((R)-1-(4-(1H-imidazol-4-yl)thiophen-2-yl)ethyl)-2-methylpropane-2-sulfinamide (80 mg, 0.27 mmol) in DCM (2.0 mL) was added dropwise HCl/1,4-dioxane (2.0 mL) at room temperature and the mixture was stirred for 2 hours. The reaction mixture was concentrated to give (R)-1-(4-(1H-imidazol-4-yl)thiophen-2-yl)ethan-1-amine hydrochloride (62 mg, 100% yield) as a brown solid, which was used directly in next step without further purification. LC/MS (ESI) (m/z): 177 (M−NH₂)⁺.

### Step 3: (S)—N—((R)-1-(4-(1H-imidazol-4-yl)thiophen-2-yl)ethyl)-7-((9,9-difluoro-9H-fluorene-3-carbonyl)glycyl)-1,4-dioxa-7-azaspiro[4.4]nonane-8-carboxamide (Compound 96)

To a mixture of (1 S,3S,5S)-5-methyl-2-((4-phenoxybenzoyl)glycyl)-2-azabicyclo[3.1.0]hexane-3-carboxylic acid (33 mg, 0.07 mmol), (R)-1-(4-(1H-imidazol-4-yl)thiophen-2-yl)ethan-1-amine hydrochloride (34 mg, 0.15 mmol) and DIPEA (36 mg, 0.28 mmol) in DMF (0.5 mL) was added PyBop (52 mg, 0.1 mmol) under N₂ atmosphere and the reaction mixture was stirred at room temperature for 1 hour. The mixture was diluted with ethyl acetate and washed with saturated aq·NH₄Cl solution. The aqueous layer was extracted with EtOAc twice and the combined organic layers were washed with brine, dried over anhydrous Na₂SO₄, filtered, and concentrated under reduced pressure to dryness. The residue was purified by prep-HPLC to afford Compound 96 (17 mg, yield 37.3%) as a white solid. ¹H NMR (400 MHz, CD₃OD) δ 8.35 (s, 1H), 8.15 (s, 1H), 7.85 (d, J=7.7 Hz, 1H), 7.72-7.61 (m, 4H), 7.55 (t, J=7.5 Hz, 1H), 7.45-7.35 (m, 3H), 7.30 (s, 1H), 5.41-5.09 (m, 1H), 4.65-4.52 (m, 1H), 4.21 (q, J=16.6 Hz, 2H), 3.99 (dd, J=10.4, 7.7 Hz, 4H), 3.80 (s, 1H), 3.20 (dt, J=27.4, 13.7 Hz, 1H), 2.44 (dd, J=13.1, 9.1 Hz, 1H), 2.23 (dd, J=13.2, 6.2 Hz, 1H), 1.61 (dd, J=32.5, 6.9 Hz, 3H). LC/MS (ESI) m/z: 634 (M+H)⁺.

### Scheme 96: Synthesis of (S)—N—((R)-1-(4-(1H-pyrazol-3-yl)thiophen-2-yl)ethyl)-7-((9,9-difluoro-9H-fluorene-3-carbonyl)glycyl)-1,4-dioxa-7-azaspiro[4.4]nonane-8-carboxamide (Compound 97)

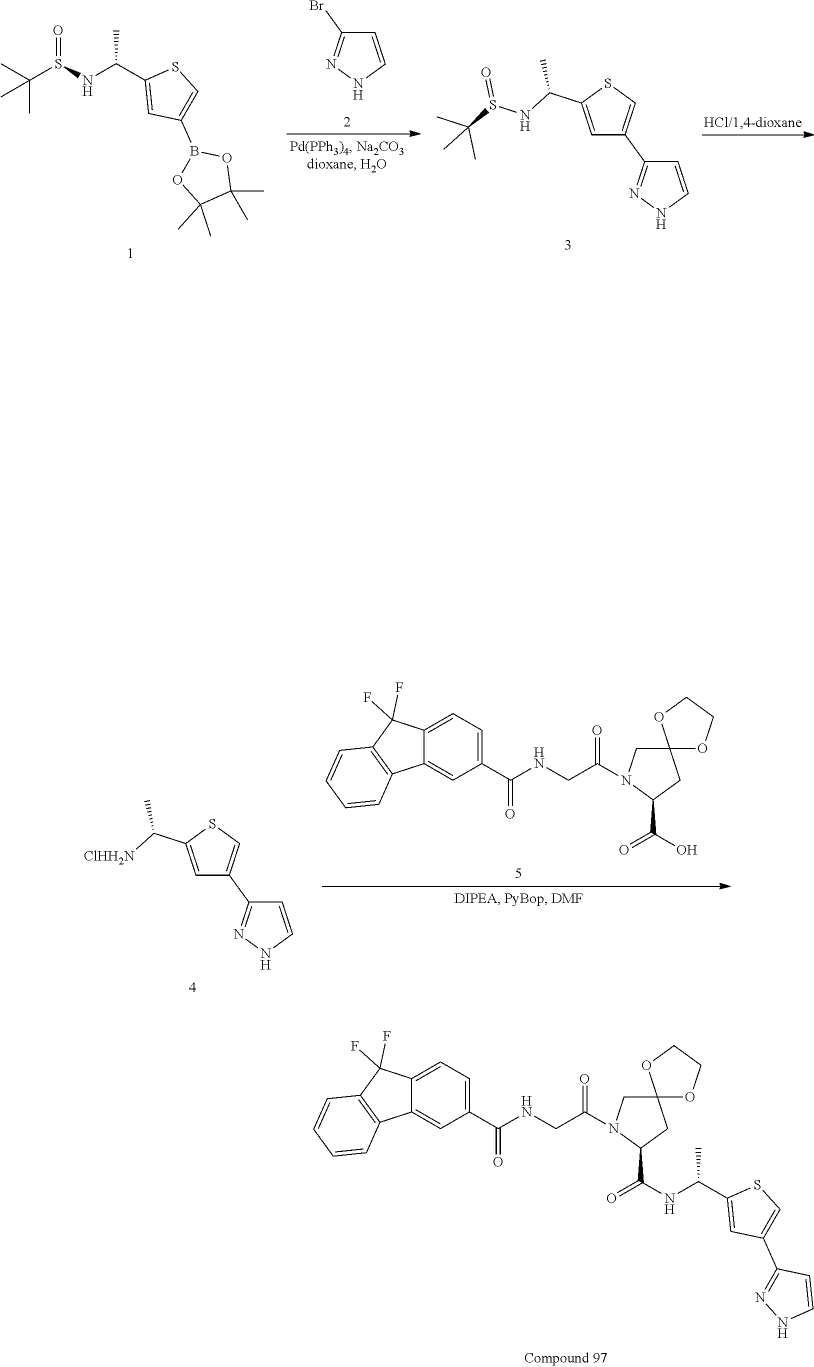

Compound 97 was prepared as a white solid from (R)-2-methyl-N—((R)-1-(4-(4,4,5,5-tetramethyl-1,3,2-dioxaborolan-2-yl)thiophen-2-yl)ethyl)propane-2-sulfinamide, 3-bromo-1H-pyrazole (83 mg, 0.56 mmol), and (1S,3S,5S)-5-methyl-2-((4-phenoxybenzoyl)glycyl)-2-azabicyclo[3.1.0]hexane-3-carboxylic acid based on the procedures set forth in Scheme 95. ¹H NMR (400 MHz, CD₃OD) δ 8.54 (s, 1H), 8.16 (d, J=13.0 Hz, 1H), 7.87 (d, J=8.0 Hz, 1H), 7.75-7.67 (m, 2H), 7.64 (d, J=7.3 Hz, 1H), 7.49 (ddd, J=24.1, 15.2, 7.5 Hz, 5H), 6.53 (s, 1H), 5.31 (d, J=7.2 Hz, 1H), 4.63-4.56 (m, 1H), 4.21 (q, J=16.7 Hz, 2H), 3.97 (d, J=18.8 Hz, 4H), 3.80 (s, 1H), 3.13 (s, 1H), 2.44 (dd, J=13.1, 9.2 Hz, 1H), 2.23 (dd, J=13.2, 6.4 Hz, 1H), 1.60 (t, J=17.0 Hz, 3H). LC/MS (ESI) m/z: 634 (M+H)⁺.

### Scheme 97: Synthesis of (2S,4R)—N—((R)-1-(4-carbamimidoylthiophen-2-yl)ethyl)-4-cyclohexyl-1-((9,9-difluoro-9H-fluorene-3-carbonyl)glycyl)pyrrolidine-2-carboxamide (Compound 98)

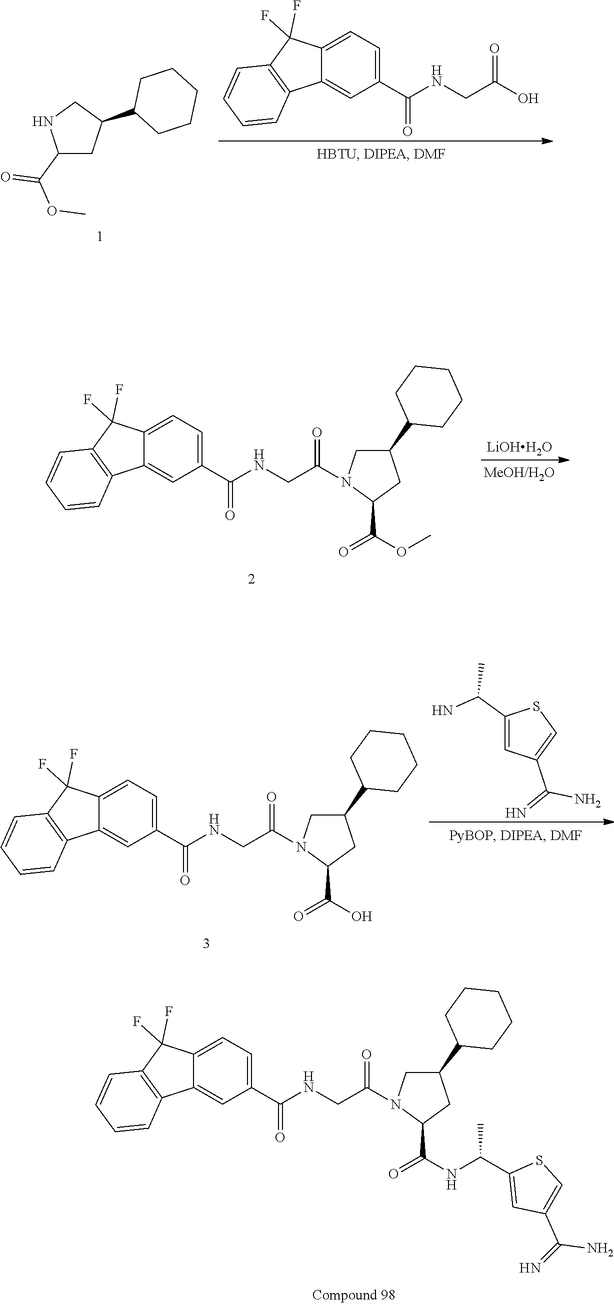

Compound 98 was prepared as a white solid from methyl (2S,4R)-4-cyclohexylpyrrolidine-2-carboxylate, (9,9-difluoro-9H-fluorene-3-carbonyl)glycine (57 mg, 0.19 mmol), and (R)-5-(1-aminoethyl) thiophene-3-carboximidamide based on the procedures set forth in Scheme 3. ¹H NMR (400 MHz, CD₃OD) δ 8.49 (s, 1H), 8.22 (dd, J=14.9, 13.5 Hz, 2H), 7.87 (t, J=7.4 Hz, 1H), 7.73 (dd, J=18.3, 7.8 Hz, 2H), 7.65 (d, J=7.3 Hz, 1H), 7.57 (t, J=7.4 Hz, 1H), 7.49-7.42 (m, 2H), 5.30 (dq, J=13.7, 6.8 Hz, 1H), 4.59 (dd, J=24.5, 8.0 Hz, 2H), 4.29-4.13 (m, 2H), 3.96 (t, J=8.5 Hz, 1H), 2.27-2.12 (m, 2H), 1.91 (dd, J=21.5, 12.2 Hz, 1H), 1.72 (dd, J=26.4, 11.3 Hz, 5H), 1.58 (d, J=7.0 Hz, 3H), 1.28 (dd, J=21.9, 9.5 Hz, 4H), 1.04 (dd, J=22.6, 11.4 Hz, 2H). LC/MS (ESI) (m/z): 634 (M+H)⁺.

### Scheme 98: Synthesis of (8S)—N-[(1R)-1-(4-carbamimidoylthiophen-2-yl)ethyl]-7-{2-[(9-methyl-9H-fluoren-3-yl)formamido]acetyl}-1,4-dioxa-7-azaspiro[4.4]nonane-8-carboxamide (Compound 99)

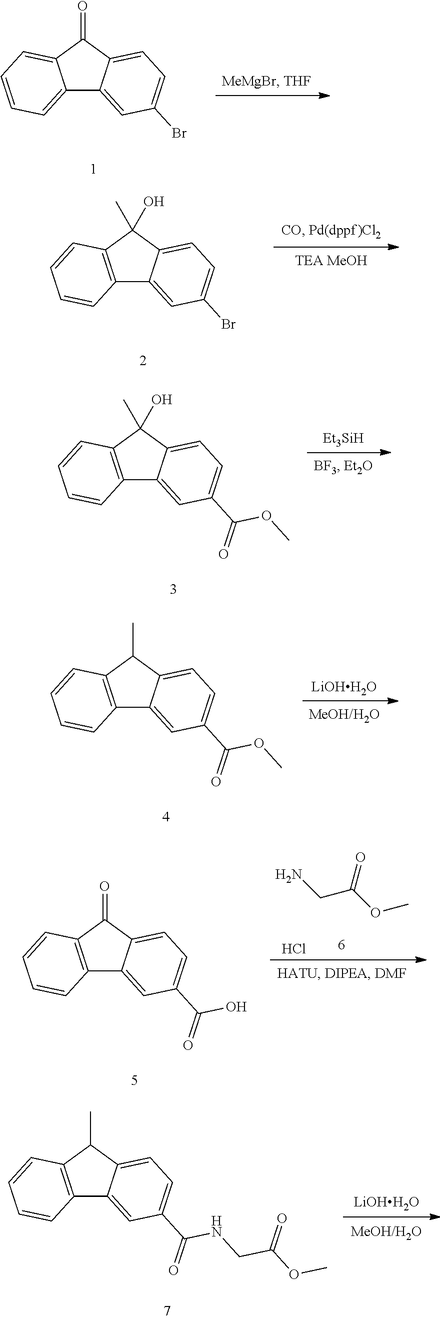

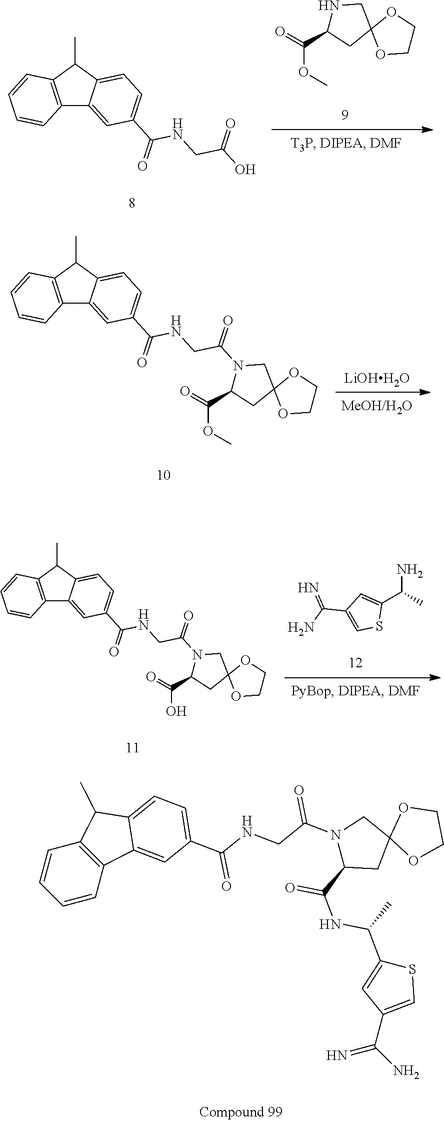

### Step 1: 3-bromo-9-methylfluoren-9-ol (2)

To a solution of 3-bromofluoren-9-one (750 mg, 2.90 mmol) in THF (10 mL) was added MeMgBr (6 mL, 5.79 mmol) at −78° C. The mixture was stirred at −78° C. under N₂ atmosphere for 3 hours. The mixture was diluted with water and extracted with EtOAc (3×50 mL). The combined organic layers were washed with brine, dried over anhydrous Na₂SO₄, filtered, and concentrated to dryness under reduced pressure. The residue was purified by flash chromatography on silica gel (PE:EtOAc=5:1) to give 3-bromo-9-methylfluoren-9-ol (780 mg, yield 97.9%) as a white solid. LC/MS (ESI) m/z: 257 (M-OH)⁺.

### Step 2: methyl 9-hydroxy-9-methylfluorene-3-carboxylate (3)

To a solution of 3-bromo-9-methylfluoren-9-ol (700 mg, 2.54 mmol) in MeOH (20 mL) was added Pd(dppf)Cl₂ (93 mg, 0.13 mmol) and TEA (771 mg, 7.63 mmol). The mixture was stirred at 70° C. under CO atmosphere overnight. The reaction mixture was filtered and concentrated under reduced pressure. The residue was purified by flash chromatography on silica gel (PE:EtOAc=3:1) to give methyl 9-hydroxy-9-methylfluorene-3-carboxylate (500 mg, yield 77.3%) as a white solid. LC/MS (ESI) m/z: 237 (M-OH)⁺.

### Step 3: methyl 9-methyl-9H-fluorene-3-carboxylate (4)

To a solution of methyl 9-hydroxy-9-methylfluorene-3-carboxylate (400 mg, 1.57 mmol) in THF (10 mL) was added Et₃SiH (454 mg, 3.93 mmol) at 0° C. and the mixture was stirred for 10 minutes. The reaction mixture was added BF₃·Et₂O (559 mg, 3.93 mmol) and the mixture was stirred at room temperature for 4 hours. The reaction mixture was quenched with water and extracted with EtOAc twice (2×30 mL). The combined organic layers were washed with brine, dried over anhydrous Na₂SO₄, filtered, and concentrated to dryness under reduced pressure. The residue was purified by flash chromatography on silica gel (PE:EtOAc=3:1) to afford methyl 9-methyl-9H-fluorene-3-carboxylate (300 mg, yield 80.0%) as a white solid. LC/MS (ESI) (m/z): 239(M+H)⁺.

### Step 4: 9-methyl-9H-fluorene-3-carboxylic acid (5)

To a solution of methyl 9-methyl-9H-fluorene-3-carboxylate (200 mg, 0.84 mmol) in MeOH (1.6 mL) and H₂O (0.4 mL) was added LiOH·H₂O (106 mg, 2.52 mmol) at 0° C., and the mixture was stirred at room temperature overnight. The mixture was acidified with 1 N aq·HCl solution to pH<3 and extracted with EtOAc twice (2×50 mL). The combined organic layers were concentrated to dryness under reduced pressure to give 9-methyl-9H-fluorene-3-carboxylic acid (180 mg, yield 95.5%) as a white solid. LC/MS (ESI) (m/z): 225 (M+H)⁺.

### Step 5: methyl (9-methyl-9H-fluorene-3-carbonyl)glycinate (7)

To a solution of 9-methyl-9H-fluorene-3-carboxylic acid (180 mg, 0.8 mmol) in DMF (2 mL) was added methyl glycinate hydrochloride (151 mg, 1.2 mmol), HATU (365 mg, 0.96 mmol) and DIPEA (310 mg, 2.4 mmol) under N₂ atmosphere and the reaction mixture was stirred at room temperature for 1 hour. The mixture was diluted with EtOAc and washed with brine, dried over anhydrous Na₂SO₄, filtered, and concentrated under reduced pressure to dryness. The residue was purified by flash chromatography on silica gel (PE:EtOAc=5:1) to afford methyl (9-methyl-9H-fluorene-3-carbonyl)glycinate (205 mg, yield 86.9%) as a white solid. LC/MS (ESI) m/z: 296 (M+H)⁺.

### Step 6: (9-methyl-9H-fluorene-3-carbonyl)glycine (8)

To a mixture of methyl (9-methyl-9H-fluorene-3-carbonyl)glycinate (200 mg, 0.68 mmol) in MeOH (1.6 mL) and H₂O (0.4 mL) was added LiOH·H₂O (85 mg, 2.03 mmol) and the reaction mixture was stirred at room temperature for 4 hours. The mixture was acidified with 1 N aq·HCl to pH-4 and extracted with EtOAc twice. The combined organic layers were washed with brine, dried over anhydrous Na₂SO₄, filtered, and concentrated to dryness under reduced pressure to afford (9-methyl-9H-fluorene-3-carbonyl)glycine (180 mg, yield 94.5%) as a white solid which was used in next step without purification. LC/MS (ESI) m/z: 282 (M+H)⁺.

### Step 7: methyl (S)-1,4-dioxa-7-azaspiro[4.4]nonane-8-carboxylate (9)

To a mixture of 7-benzyl 8-methyl(8S)-1,4-dioxa-7-azaspiro[4.4]nonane-7,8-dicarboxylate (199 mg, 0.62 mmol) in MeOH (5 mL) was added Pd/C (16 mg, 10% wt.) and the mixture was degassed under N₂ atmosphere for three times and stirred under a H₂ balloon at 25° C. overnight. The mixture was filtered and concentrated to dryness under reduced pressure to afford methyl (S)-1,4-dioxa-7-azaspiro[4.4]nonane-8-carboxylate (116 mg, yield 86.7%) as a yellow oil, which was used in next step without purification. LC/MS (ESI) m/z: 188 (M+H)⁺.

### Step 8: methyl (8S)-7-{2-[(9-methyl-9H-fluoren-3-yl)formamido]acetyl}-1,4-dioxa-7-azaspiro[4.4]nonane-8-carboxylate (10)

To a solution of (9-methyl-9H-fluorene-3-carbonyl)glycine (147 mg, 0.52 mmol), methyl (S)-1,4-dioxa-7-azaspiro[4.4]nonane-8-carboxylate (116 mg, 0.62 mmol) and DIPEA (207 mg, 1.6 mmol) in DMF (1.5 mL) was added T₃P (395 mg, 0.62 mmol, 50% in EtOAc) under N₂ atmosphere and the reaction mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography on silica gel (PE:EtOAc=3:1) to afford methyl (8S)-7-{2-[(9-methyl-9H-fluoren-3-yl)formamido]acetyl}-1,4-dioxa-7-azaspiro[4.4]nonane-8-carboxylate (200 mg, yield 85.2%) as a yellow oil. LC/MS (ESI) m/z: 451 (M+H)⁺.

### Step 10: (8S)-7-((9-methyl-9H-fluorene-3-carbonyl)glycyl)-1,4-dioxa-7-azaspiro[4.4]nonane-8-carboxylic acid (11)

To a mixture of methyl (8S)-7-((9-methyl-9H-fluorene-3-carbonyl)glycyl)-1,4-dioxa-7-azaspiro[4.4]nonane-8-carboxylate (200 mg, 0.44 mmol) in MeOH (1.6 mL) and H₂O (0.4 mL) was added LiOH·H₂O (75 mg, 1.78 mmol) at 0° C., and the reaction mixture was stirred at room temperature for 4 hours. The mixture was acidified with 1 N aq·HCl to pH˜4 and extracted with EtOAc twice. The combined organic layers were washed with brine, dried over anhydrous Na₂SO₄, filtered, and concentrated to dryness under reduced pressure to afford (8S)-7-((9-methyl-9H-fluorene-3-carbonyl)glycyl)-1,4-dioxa-7-azaspiro[4.4]nonane-8-carboxylic acid (180 mg, yield 93.8%) as a white solid which was used in next step without purification LC/MS (ESI) m/z: 437 (M+H)⁺.

### Step 11: (8S)—N-[(1R)-1-(4-carbamimidoylthiophen-2-yl)ethyl]-7-(2-[(9-methyl-9H-fluoren-3-yl)formamido]acetyl)-1,4-dioxa-7-azaspiro[4.4]nonane-6-carboxamide (Compound 99)

To a mixture of (8S)-7-{2-[(9-methyl-9H-fluoren-3-yl)formamido]acetyl}-1,4-dioxa-7-azaspiro[4.4]nonane-8-carboxylic acid (35 mg, 0.08 mmol), 5-[(1R)-1-aminoethyl]thiophene-3-carboximidamide (21 mg, 0.12 mmol) and DIPEA (31 mg, 0.24 mmol) in DMF (0.5 mL) was added PyBop (52 mg, 0.1 mmol) under N₂ atmosphere at 0° C., and the reaction mixture was stirred at room temperature for 1 hour. The mixture was diluted with EtOAc and washed with saturated aq·NH₄Cl solution. The aqueous layer was extracted with EtOAc twice and the combined organic layers were washed with brine, dried over anhydrous Na₂SO₄, filtered, and concentrated under reduced pressure to dryness. The residue was purified by prep-HPLC to afford Compound 99 (4.9 mg, 0.008 mmol, yield 10.4%) as a white solid. ¹H NMR (400 MHz, CD₃OD) δ 8.49 (s, 1H), 8.35-8.13 (m, 2H), 7.83 (d, J=7.7 Hz, 2H), 7.71-7.46 (m, 3H), 7.38 (p, J=7.4 Hz, 2H), 5.29 (d, J=7.1 Hz, 1H), 4.75-4.51 (m, 1H), 4.21 (s, 2H), 3.99 (dd, J=14.8, 6.0 Hz, 5H), 3.82 (q, J=10.9 Hz, 2H), 2.42 (dd, J=27.9, 19.0 Hz, 1H), 2.22 (dd, J=13.1, 6.6 Hz, 1H), 1.62 (t, J=18.5 Hz, 3H), 1.53 (d, J=7.4 Hz, 3H). LC/MS (ESI) m/z: 588 (M+H)⁺.

### Scheme 99: Synthesis of (S)—N—((R)-1-(4-(2-aminopyrimidin-5-yl)thiophen-2-yl) ethyl)-7-((9,9-difluoro-9H-fluorene-3-carbonyl)glycyl)-1,4-dioxa-7-azaspiro[4.4]nonane-8-carboxamide (Compound 100)

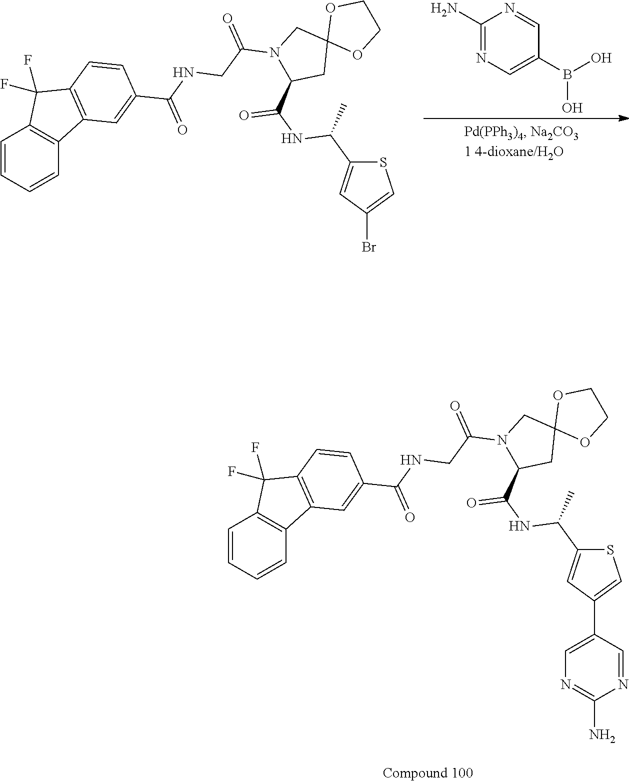 [SMILES: C[C@@H](NC(=O)[C@@H]1CC2(CN1C(=O)CNC(=O)c1ccc3c(c1)-c1ccccc1C3(F)F)OCCO2)c1cc(Br)cs1 ; Nc1ncc(B(O)O)cn1 ; C[C@@H](NC(=O)[C@@H]1CC2(CN1C(=O)CNC(=O)c1ccc3c(c1)-c1ccccc1C3(F)F)OCCO2)c1cc(-c2cnc(N)nc2)cs1]

To a solution of (S)—N—((R)-1-(4-bromothiophen-2-yl)ethyl)-7-((9,9-difluoro-9H-fluorene-3-carbonyl)glycyl)-1,4-dioxa-7-azaspiro[4.4]nonane-8-carboxamide (40 mg, 0.062 mmol) in 1,4-dioxane (1 mL) and water (0.3 mL) was added (2-aminopyrimidin-5-yl)boronic acid (17 mg, 0.124 mmol), Na₂CO₃ (197 mg, 1.86 mmol) and Pd(PPh₃)₄ (7 mg, 0.0062 mmol) at room temperature. The mixture was degassed under N₂ atmosphere for three times and stirred under a N₂ balloon at 90° C. overnight. The mixture was diluted with EtOAc, washed with water and brine, dried over anhydrous Na₂SO₄, filtered, and concentrated under reduced pressure to dryness. The residue was purified by prep-HPLC to give Compound 100 (2.0 mg, yield 4.9%) as a white solid. ¹H NMR (400 MHz, DMSO) δ 8.85 (ddd, J=43.6, 25.2, 7.1 Hz, 1H), 8.57 (d, J=3.2 Hz, 2H), 8.37-8.24 (m, 2H), 7.92-7.81 (m, 3H), 7.75 (d, J=7.1 Hz, 1H), 7.62 (dd, J=14.3, 7.7 Hz, 2H), 7.48 (t, J=7.5 Hz, 1H), 7.41 (s, 1H), 6.66 (d, J=17.7 Hz, 2H), 5.19 (dt, J=14.0, 6.7 Hz, 1H), 4.56 (dt, J=15.3, 7.0 Hz, 1H), 4.20 (td, J=17.1, 6.0 Hz, 1H), 4.10-3.75 (m, 6H), 3.66 (d, J=10.6 Hz, 1H), 2.34 (t, J=11.1 Hz, 1H), 2.13 (ddd, J=20.5, 13.0, 7.4 Hz, 1H), 1.49 (dd, J=37.2, 6.9 Hz, 3H). LC/MS (ESI) m/z: 661 (M+H)⁺.

### Scheme 100: Synthesis of (S)—N—((R)-1-(4-carbamimidoylthiophen-2-yl)ethyl)-7-((4′-fluoro-[1,1′-biphenyl]-4-carbonyl)glycyl)-1,4-dioxa-7-azaspiro[4.4]nonane-8-carboxamide (Compound 101)

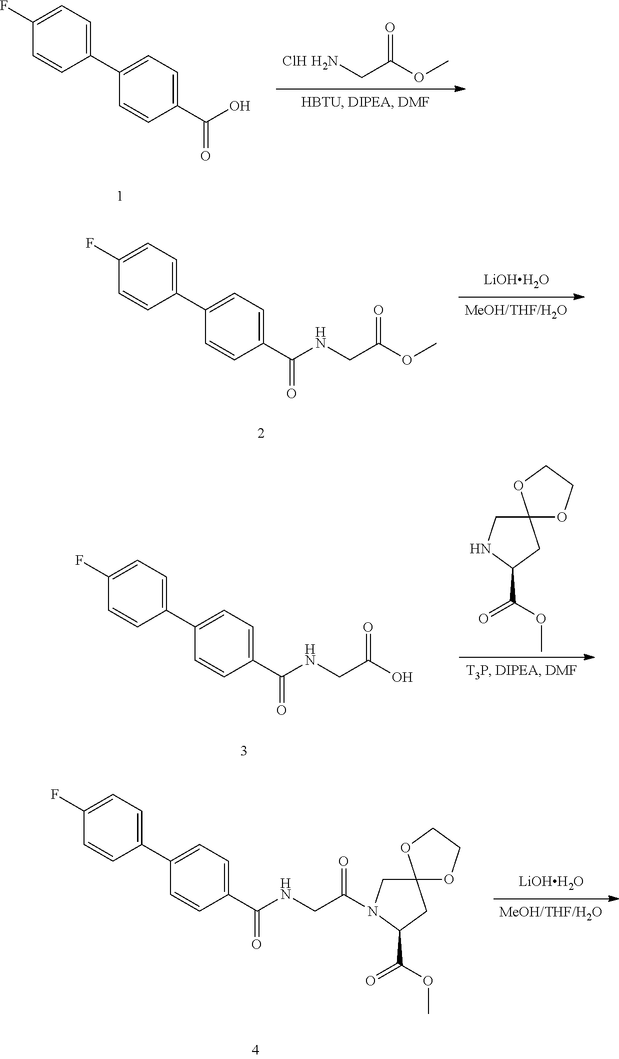

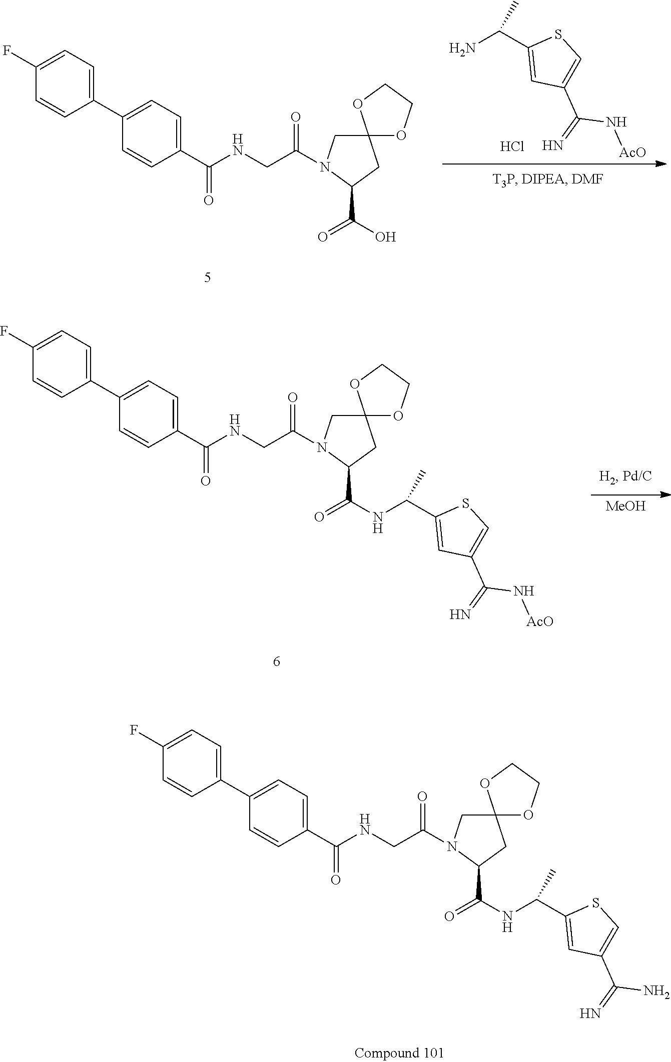

Compound 101 was prepared from 4′-fluoro-[1,1′-biphenyl]-4-carboxylic acid, methyl glycinate hydrochloride, methyl (S)-1,4-dioxa-7-azaspiro[4.4]nonane-8-carboxylate, and (R)—N-acetoxy-5-(1-aminoethyl)thiophene-3-carboximidamide hydrochloride based on the procedures set forth in Scheme 89. ¹H NMR (400 MHz, CD₃OD) δ 8.54 (s, 1H), 8.24 (d, J=8.2 Hz, 1H), 7.93 (d, J=8.5 Hz, 2H), 7.70 (t, J=8.5 Hz, 4H), 7.53 (d, J=7.3 Hz, 1H), 7.21 (t, J=8.7 Hz, 2H), 5.28 (q, J=6.4 Hz, 1H), 4.60-4.55 (m, 1H), 4.18 (s, 2H), 4.02-3.94 (m, 4H), 3.80 (q, J=10.8 Hz, 2H), 2.48-2.38 (m, 1H), 2.21 (dd, J=13.1, 6.5 Hz, 1H), 1.63 (dd, J=37.0, 7.1 Hz, 3H). LC/MS (ESI) m/z: 580 (M+H)⁺.

### Scheme 101: (S)—N-((4-carbamimidoylthiophen-2-yl)methyl)-7-((3-fluoro-5-(p-tolyl) picolinoyl)glycyl)-1,4-dioxa-7-azaspiro[4.4]nonane-8-carboxamide (Compound 102)

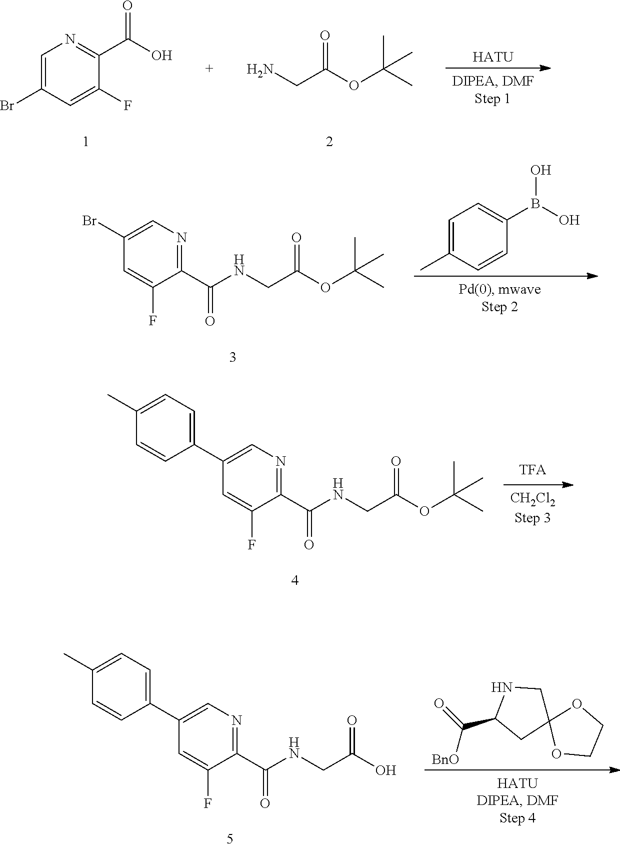

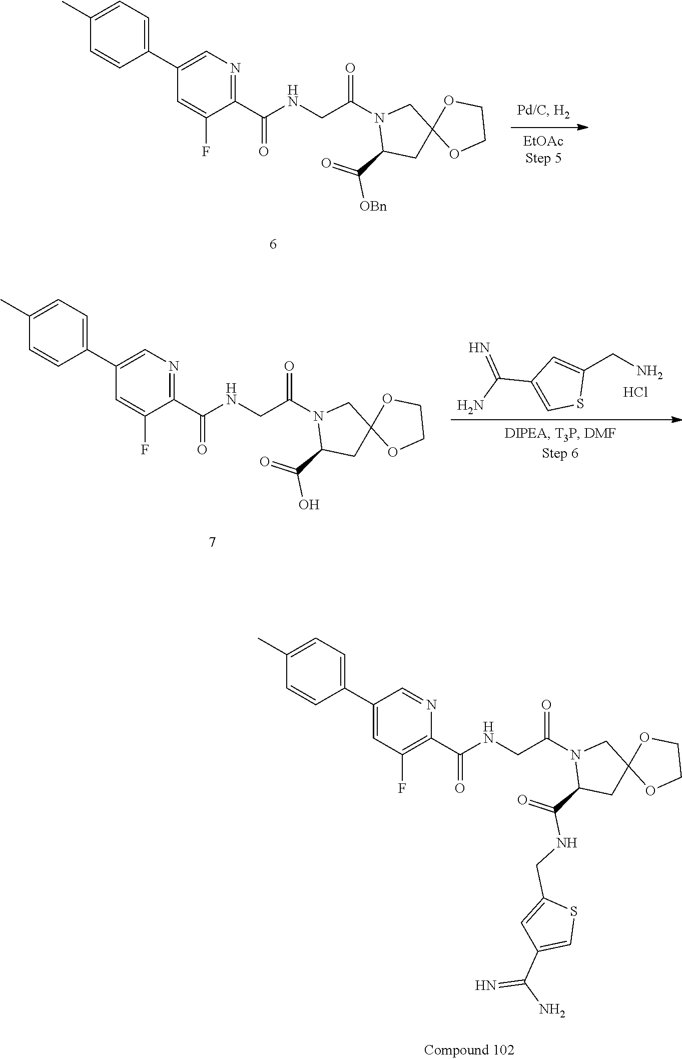 [SMILES: Cc1ccc(-c2cnc(C(=O)NCC(=O)N3CC4(C[C@H]3C(=O)OCc3ccccc3)OCCO4)c(F)c2)cc1 ; Cc1ccc(-c2cnc(C(=O)NCC(=O)N3CC4(C[C@H]3C(=O)O)OCCO4)c(F)c2)cc1 ; N=C(N)c1csc(CN)c1 ; Cl ; Cc1ccc(-c2cnc(C(=O)NCC(=O)N3CC4(C[C@H]3C(=O)NCc3cc(C(=N)N)cs3)OCCO4)c(F)c2)cc1]

### Step 1: tert-butyl (5-bromo-3-fluoropicolinoyl)glycinate (3)

To a solution of 5-bromo-3-fluoro-pyridine-2-carboxylic acid (1.0 g, 4.55 mmol, 1 equiv.) in DMF (5 mL) was added tert-butyl 2-aminoacetate. HCl (0.91 g, 5.46 mmol, 1.2 equiv.), HATU (2.25 g, 5.91 mmol, 1.3 equiv.) and DIPEA (2.38 mL, 13.6 mmol, 3 equiv.). The reaction was stirred at room temperature for 1 hour. Water (20 mL) was added to reaction and the mixture was then stirred for 10 minutes. The product crashed out and the solid was collected by filtration and dried to give 3 (1.25 g, 3.74 mmol, yield 82.22%) as a yellow oil. LC/MS (ESI) m/z: 334 (M+H)⁺.

### Step 2: tert-butyl (3-fluoro-5-(p-tolyl)picolinoyl)glycinate (4)

A mixture of p-tolylboronic acid (0.10 g, 0.74 mmol, 1.2 equiv.) and compound 3 (0.30 g, 0.90 mmol, 1 equiv.) Na₂CO₃ (0.20 g, 1.89 mmol, 2 equiv.) and Pd(dpp)Cl₂-DCM (0.07 g, 0.09 mmol) in water (0.3 ml) and 1,4-dioxane (3 mL) was placed in a microwave reactor for 30 minutes at 110° C.

The reaction was cooled to room temperature, diluted with water and EtOAc. The two layers were separated, and the aq. Layer extracted with EtOAc (5 mL×2), washed with brine, dried over Na₂SO₄ and concentrated. Purification by Combi Flash: 12 g column; solvent A=Hexanes, solvent B=EtOAc, 100% A to 25% B gave 4 (0.29 g, 0.84 mmol, 93.50% yield) as a yellow oil. LC/MS (ESI) m/z: 345 (M+H)+.

### Step 3: (3-fluoro-5-(p-tolyl)picolinoyl)glycine (5)

TFA (0.64 mL) was added to a solution of 4 (0.29 g, 0.84 mmol, 1.0 equiv.) in CH₂Cl₂ (2 mL) at ice-bath temperature. The reaction was stirred at room temperature for 3 hours and then concentrated to dryness to give 5 (0.21 g, 0.73 mmol, yield 86.50%) as a yellow solid which was used in the next step without further purification. LC/MS (ESI) m/z: 289 (M+H)⁺.

### Step 4: benzyl (S)-7-((3-fluoro-5-(p-tolyl)picolinoyl)glycyl)-1,4-dioxa-7-azaspiro[4.4]nonane-8-carboxylate (6)

To a solution of 5 (0.2 g, 0.69 mmol, 1.0 equiv.) in DMF (2 mL) was added benzyl (8S)-1,4-dioxa-7-azaspiro[4.4]nonane-8-carboxylate (0.2 g, 0.76 mmol, 1.1 equiv.), HATU (0.34 g, 0.90 mmol, 1.3 equiv.) and DIPEA (0.36 mL, 2.08 mmol, 3.0 equiv.). The reaction was stirred at room temperature for 30 minutes. Water (5 mL) was added, and the liquid was decanted. Water (5 mL) and EtOAc (5 mL) and was added the residue. The two layers were separated, and the organic layer washed with brine, dried over Na₂SO₄, and concentrated to give 6 as a brown solid which was used without any further purification (0.25 g, 0.47 mmol, yield 81.04%). LC/MS (ESI) m/z: 534 (M+H)⁺.

### Step 5: (S)-7-((3-fluoro-5-(p-tolyl)picolinoyl)glycyl)-1,4-dioxa-7-azaspiro[4.4]nonane-8-carboxylic acid (7)

To a solution of 6 (0.2 g, 0.38 mmol, 1.0 equiv.) in MeOH (3 mL) was added 10% Pd/C (0.1 g, 0.094 mmol, 0.1 equiv.). The flask was evacuated and then backfilled with H₂ in a balloon. The reaction was stirred at room temperature overnight and then filtered through Celite pad. The filtrate was concentrated and dried in vacuo to give 7 as a yellow solid which was used without further purification. LC/MS (ESI) m/z: 444 (M+H)+.

### Step 6: (S)—N-((4-carbamimidoylthiophen-2-yl)methyl)-7-((3-methyl-5-(p-tolyl)picolinoyl) glycyl)-1,4-dioxa-7-azaspiro[4.4]nonane-6-carboxamide (Compound 102)

To a solution of 7 (60 mg, 0.14 mmol, 1.0 equiv.) in DMF (5 mL) was added 5-(aminomethyl)thiophene-3-carboximidamide. HCl (30 mg, 0.16 mmol, 1.1 equiv.), T3P (50 mass %) in EtOAc (0.10 mL, 0.17 mmol, 1.5 equiv.), and DIPEA (0.07 mL, 0.40 mmol, 3.0 equiv.). The reaction mixture was stirred at room temperature for 1 hour and then purified directly by HPLC to give Compound 102 (7.5 mg, 0.014 mmol, 9.55% yield) LC/MS (ESI) m/z: 581 (M+H)+. ¹H NMR (400 MHz, DMSO-d₆) δ 8.85 (s, 1H), 8.67 (s, 1H), 8.33 (s, 1H), 8.18 (d, J=12.2 Hz, 1H), 7.78 (d, J=7.5 Hz, 2H), 7.36 (d, J=7.5 Hz, 2H) 4.69-4.34 (m, 3H), 4.24-4.17 (m, 2H), 4.14 (dd, J=16.6, 5.6 Hz, 2H), 4.04-3.81 (m, 2H), 3.79-3.13 (m, 2H), 2.78-2.65 (m, 2H), 2.36 (s, 3H), 2.04 (dt, J=12.1, 6.0 Hz, 1H).

### Scheme 102: (S)—N-((4-(6-aminopyridin-2-yl)thiophen-2-yl)methyl)-7-((4-(4-fluorophenoxy)benzoyl)glycyl)-1,4-dioxa-7-azaspiro[4.4]nonane-8-carboxamide (Compound 103)

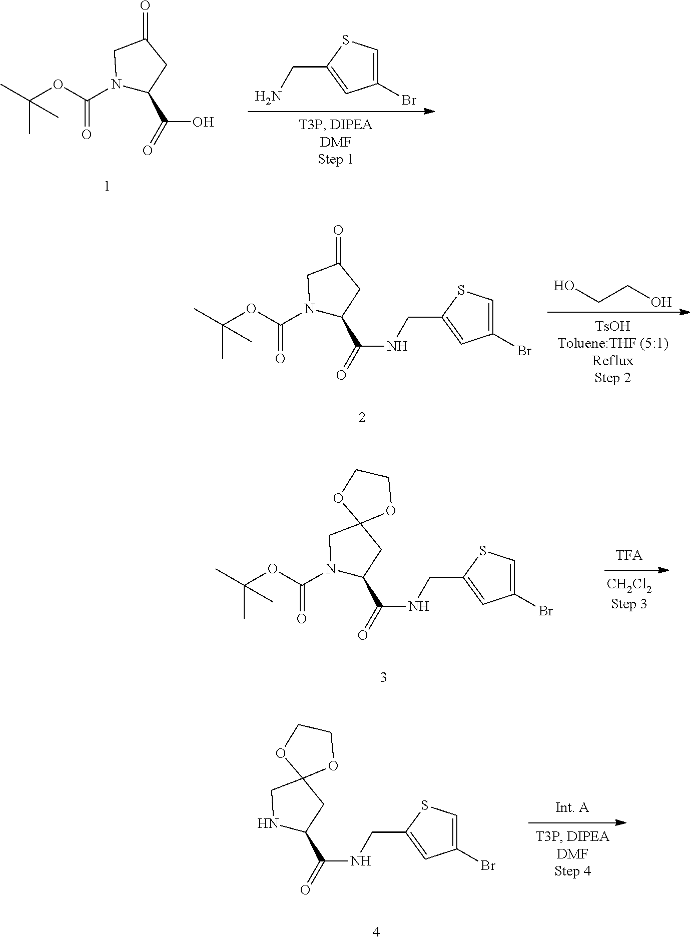

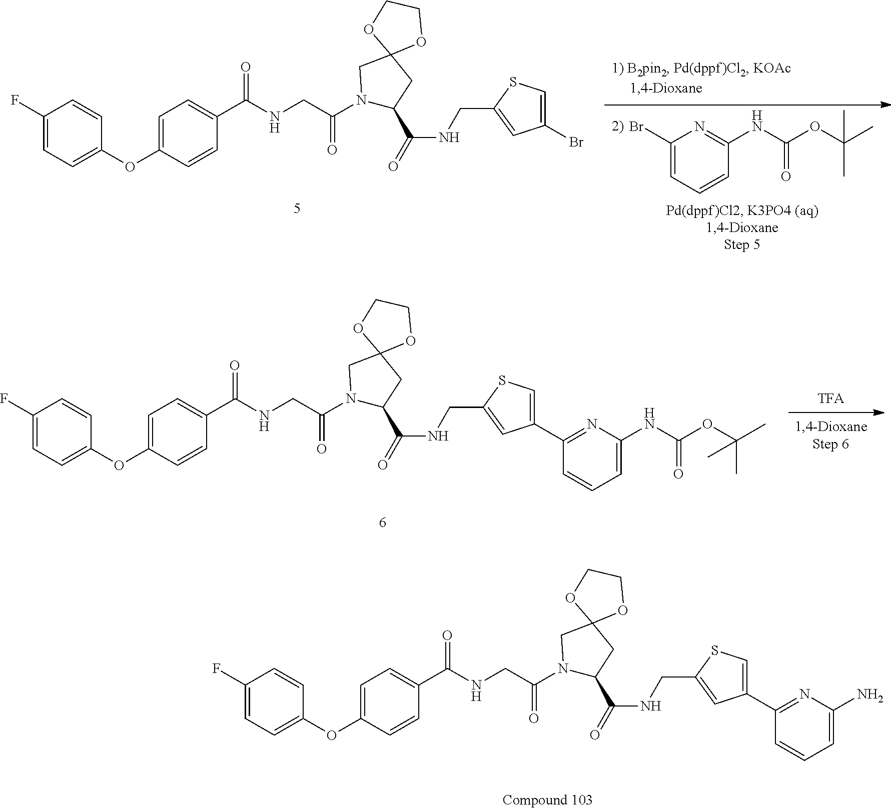 [SMILES: O=C(NCC(=O)N1CC2(C[C@H]1C(=O)NCc1cc(Br)cs1)OCCO2)c1ccc(Oc2ccc(F)cc2)cc1 ; CC(C)(C)OC(=O)Nc1cccc(Br)n1 ; CC(C)(C)OC(=O)Nc1cccc(-c2csc(CNC(=O)[C@@H]3CC4(CN3C(=O)CNC(=O)c3ccc(Oc5ccc(F)cc5)cc3)OCCO4)c2)n1 ; Nc1cccc(-c2csc(CNC(=O)[C@@H]3CC4(CN3C(=O)CNC(=O)c3ccc(Oc5ccc(F)cc5)cc3)OCCO4)c2)n1]

### Step 1: tert-butyl (S)-2-(((4-bromothiophen-2-yl)methyl)carbamoyl)-4-oxopyrrolidine-1-carboxylate (2)

To a solution of (4-bromo-2-thienyl)methanamine (3.000 g, 15.62 mmol, 1.0 equiv.) and (S)-1-(tert-butoxycarbonyl)-4-oxopyrrolidine-2-carboxylic acid, 1 (5.40 g, 23.6 mmol, 1.5 equiv.) in DMF (25.00 mL) was added DIPEA (8.0 mL, 46 mmol, 2.9 equiv.) and 1-Propanephosphonic anhydride solution (50 mass %) in Ethyl acetate, Approx 1.7 Mol/L (15.0 mL, 25.0 mmol, 1.6 equiv.) under Argon atmosphere at room temperature and it was stirred for 4 h. LC-MS indicated the completion of the reaction. The solution was then added to water while stirring. Solid formation was observed. The resulting solid was filtered and dried to obtain desired product tert-butyl (S)-2-(((4-bromothiophen-2-yl)methyl)carbamoyl)-4-oxopyrrolidine-1-carboxylate, 2 (6.299 g, 15.62 mmol, 100.0% yield). It was used to the next step without further purification. LC/MS (ESI) m/z: 404 (M+H)⁺.

### Step 2: tert-butyl (S)-8-(((4-bromothiophen-2-yl)methyl)carbamoyl)-1,4-dioxa-7-azaspiro[4.4]nonane-7-carboxylate (3)

To the solution of tert-butyl (S)-2-(((4-bromothiophen-2-yl)methyl)carbamoyl)-4-oxopyrrolidine-1-carboxylate, 2 (6.299 g, 15.62 mmol, 1.0 equiv.) in Toluene (50.00 mL) and THF (10.00 mL) was added ethylene glycol (3.50 mL, 62.6 mmol, 4.0 equiv.) and P-Toluenesulfonic acid (1.81 g, 10.4 mmol, 0.7 equiv.) at 25° C. The mixture was stirred at 130° C. for 18 hours. LC/MS indicated the completion of the reaction. The mixture was poured into aqueous NaHCO₃ (50 mL) and extracted with ethyl acetate (3×100 mL). The combined organic extracts were washed with brine, dried over Na₂SO₄ and concentrated in vacuo to obtain desired product tert-butyl (S)-8-(((4-bromothiophen-2-yl)methyl)carbamoyl)-1,4-dioxa-7-azaspiro[4.4]nonane-7-carboxylate, 3 (6.99 g, 15.62 mmol, 100% yield). It was used to the next step without further purification. LC/MS (ESI) m/z: 447 (M+H)⁺.

### Step 3: (S)—N-((4-bromothiophen-2-yl)methyl)-1,4-dioxa-7-azaspiro[4.4]nonane-6-carboxamide (4)

The product from Step 2. tert-butyl (S)-8-(((4-bromothiophen-2-yl)methyl)carbamoyl)-1,4-dioxa-7-azaspiro[4.4]nonane-7-carboxylate, 3 (6.99 g, 15.62 mmol, 1.0 equiv.) was dissolved in Dichloromethane (15.00 mL) and added Trifluoroacetic acid (10.00 mL, 132.3 mmol, 8.5 equiv.). The mixture was stirred for 1 hr at room temperature. LC-MS indicated the completion of the reaction. The reaction mixture was concentrated, and the residue was loaded to a pre-packed silica column using DCM and Triethylamine and purified by ISCO column chromatographic purification system (24 g gold) using 0-5% MeOH in DCM as eluent to provide the desired (S)—N-((4-bromothiophen-2-yl)methyl)-1,4-dioxa-7-azaspiro[4.4]nonane-8-carboxamide, 4 (3.50 g, 10.08 mmol, 64.54% yield) as orange liquid. LC/MS (ESI) m/z: 347 (M+H)⁺.

### Step 4: (S)—N-((4-bromothiophen-2-yl)methyl)-7-((4-(4-fluorophenoxy)benzoyl)glycyl)-1,4-dioxa-7-azaspiro[4.4]nonane-8-carboxamide (5)

To a solution of (4-(4-fluorophenoxy)benzoyl)glycine, Int. A (1.08 g, 3.73 mmol, 1.3 equiv.) and (S)—N-((4-bromothiophen-2-yl)methyl)-1,4-dioxa-7-azaspiro[4.4]nonane-8-carboxamide, 4 (1.00 g, 2.88 mmol, 1.0 equiv.) in DMF (15.0 mL) was added DIPEA (2.00 mL, 11.5 mmol, 4.0 equiv.) and 1-Propanephosphonic anhydride solution (50 mass %) in Ethyl acetate, Approx 1.7 Mol/L (2.60 mL, 4.33 mmol, 1.5 equiv.) under Argon atmosphere at room temperature and it was stirred for overnight. LC-MS indicated the completion of the reaction. The reaction mixture was poured into water while stirring. Solid formation was observed. The solid was filtered, and dried. The product was purified by ISCO column chromatographic purification system (40 g gold column) using 0-5% MeOH in DCM to obtain desired product (S)—N-((4-bromothiophen-2-yl)methyl)-7-((4-(4-fluorophenoxy)benzoyl)glycyl)-1,4-dioxa-7-azaspiro[4.4]nonane-8-carboxamide, 5 (0.950 g, 1.54 mmol, 53.3% yield). LC/MS (ESI) m/z: 618 (M+H)⁺.

### Step 5: tert-butyl (S)-(6-(5-((7-((4-(4-fluorophenoxy)benzoyl)glycyl)-1,4-dioxa-7-azaspiro[4.4]nonane-8-carboxamido)methyl)thiophen-3-yl)pyridin-2-yl)carbamate (6)

To a solution of (S)—N-((4-bromothiophen-2-yl)methyl)-7-((4-(4-fluorophenoxy)benzoyl)glycyl)-1,4-dioxa-7-azaspiro[4.4]nonane-8-carboxamide, 5 (0.065 g, 0.105 mmol, 1.0 equiv.) in 1,4-Dioxane (5.00 mL) was added Bis(pinacolato)diboron (0.040 g, 0.16 mmol, 1.5 equiv.) and Potassium acetate (0.020 g, 0.20 mmol, 1.9 equiv.). Ar gas was passed through the solution for 2 min followed by the addition of Pd(dppf)Cl₂·CH₂Cl₂ (0.010 g, 0.012 mmol, 0.1 equiv.). The mixture was de-gassed once again by purging with Ar. The tube was sealed and placed in a microwave synthesizer for 30 mins at 110° C. LC/MS indicated the presence of the starting material. It was irradiated with MW for another 30 mins at 110° C. to complete the reaction. To this mixture was added tert-butyl N-(6-bromo-2-pyridyl)carbamate (0.0300 g, 0.110 mmol, 1.1 equiv.) followed by Potassium phosphate tribasic solution in H₂O (0.15 mL, 0.30 mmol, 2.00 mol/L, 2.9 equiv.) and Pd(dppf)Cl₂·CH₂Cl₂ (0.010 g, 0.012 mmol, 0.1 equiv.). It was then further degassed by passing Ar through for 2 mins. The tube was sealed again, and it was irradiated with MW for another 30 mins. LC/MS showed the presence of the desired product. The reaction mixture was passed through a pad of celite and concentrated. The residue further purified by ISCO column chromatographic purification system (12 g Gold) using 0-10% MeOH in DCM as eluent to obtain the desired product tert-butyl (S)-(6-(5-((7-((4-(4-fluorophenoxy)benzoyl)glycyl)-1,4-dioxa-7-azaspiro[4.4]nonane-8-carboxamido)methyl)thiophen-3-yl)pyridin-2-yl)carbamate, 6 (0.0350 g, 0.0478 mmol, 45.5% yield). LC/MS (ESI) m/z: 732 (M+H)⁺.

### Step 6: (S)—N-((4-(6-aminopyridin-2-yl)thiophen-2-yl)methyl)-7-((4-(4-fluorophenoxy)benzoyl)glycyl)-1,4-dioxa-7-azaspiro[4.4]nonane-8-carboxamide (Compound 103)

To the solution of tert-butyl (S)-(6-(5-((7-((4-(4-fluorophenoxy)benzoyl)glycyl)-1,4-dioxa-7-azaspiro[4.4]nonane-8-carboxamido)methyl)thiophen-3-yl)pyridin-2-yl)carbamate, 6 (0.035 g, 0.048 mmol, 1.0 equiv.) in 1,4-Dioxane (4 mL) was added Trifluoroacetic acid (2.00 mL, 26.5 mmol, 550 equiv.). The mixture was stirred for 2 h. It was then concentrated and the residue was loaded to ISCO column chromatographic purification system (12 g gold) eluted with 0-10% MeOH in DCM to obtain desired product (S)—N-((4-(6-aminopyridin-2-yl)thiophen-2-yl)methyl)-7-((4-(4-fluorophenoxy)benzoyl)glycyl)-1,4-dioxa-7-azaspiro[4.4]nonane-8-carboxamide, Compound 103 (0.0120 g, 0.0190 mmol, 40% yield) as a beige solid. LC/MS (ESI) m/z: 632 (M+H)⁺. ¹H NMR (400 MHz, DMSO) δ 2.00-2.10 (dd, J=13.1, 6.9 Hz, 1H), 2.27-2.37 (dd, J=13.1, 8.8 Hz, 1H), 3.60-3.73 (m, 1H), 3.77-3.84 (d, J=10.9 Hz, 1H), 3.85-3.94 (m, 2H), 3.90-3.97 (d, J=8.2 Hz, 1H), 3.94-4.08 (m, 2H), 4.08-4.19 (dd, J=16.8, 5.8 Hz, 1H), 4.36-4.74 (m, 3H), 6.69-6.74 (s, 1H), 6.96-7.03 (t, J=8.1 Hz, 2H), 7.03-7.09 (d, J=7.8 Hz, 1H), 7.10-7.18 (m, 2H), 7.24-7.32 (t, J=8.7 Hz, 2H), 7.47-7.53 (d, J=6.4 Hz, 1H), 7.60-7.64 (s, 1H), 7.83-7.90 (m, 2H), 7.99-8.04 (s, 1H), 8.53-8.65 (m, 2H).

### Scheme 103: (S)—N-((4-carbamimidoylthiophen-2-yl)methyl)-7-((4-(1-methyl-1H-indol-5-yl)benzoyl)glycyl)-1,4-dioxa-7-azaspiro[4.4]nonane-8-carboxamide (Compound 104)

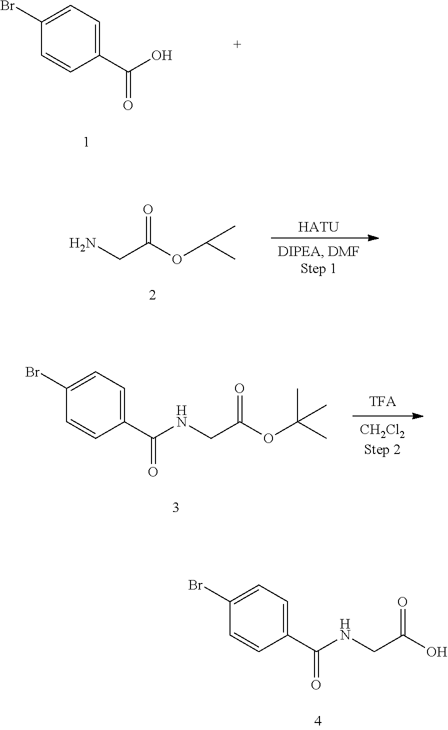

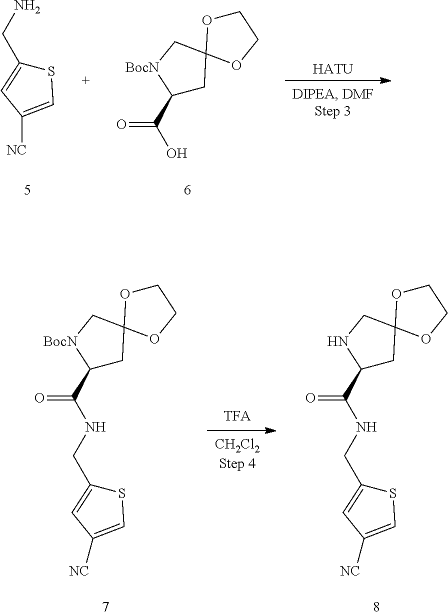

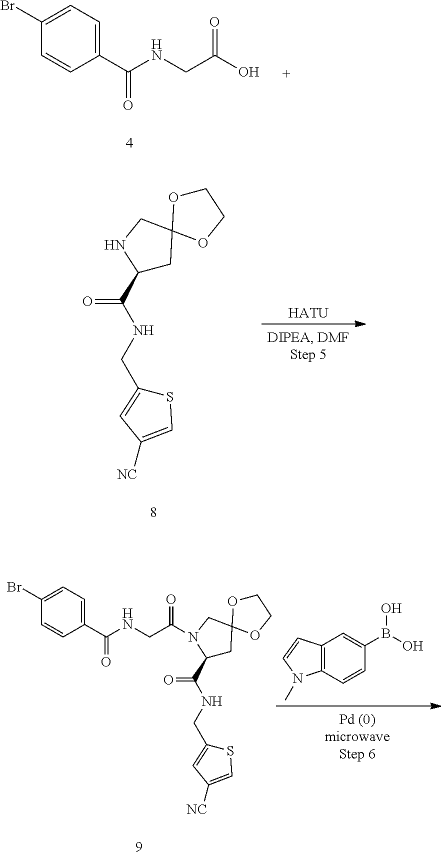

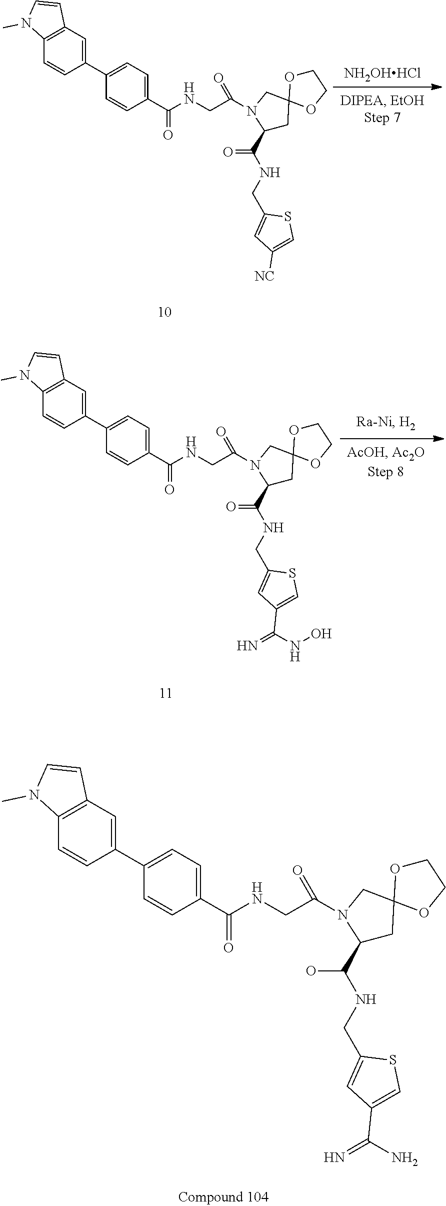

### Step 1: tert-butyl (4-bromobenzoyl)glycinate (3)

To a solution of 4-bromobenzoic acid, 1 (5 g, 24.87 mmol, 1 equiv.) in DMF (5 mL) was added tert-butyl 2-aminoacetate hydrochloride (5.4 g, 32.33 mmol, 1.3 equiv.), HATU (14.18 g, 37.31 mmol, 1.5 equiv.) and DIPEA (13.0 mL, 74.61 mmol, 3 equiv.). The reaction was stirred at room temperature for 1 hour. Water (20 mL) was added, and the mixture stirred for 10 minutes. The product crushed out and the solid was collected by filtration and dried to give 3 (7.0 g, 22.29 mmol, yield 89.63%) as a white solid. LC/MS (ESI) m/z: 315 (M+H)⁺.

### Step 2: (4-bromobenzoyl)glycine (4)

TFA (12.03 mL) was added to a solution of compound 3 (5 g, 15.92 mmol, 1.0 equiv.) in CH₂Cl₂ (20 mL) at ice-bath temperature. The reaction was stirred at room temperature for 2 hours and then concentrated to dryness to give 4 (4.0 g, 15.5 mmol, yield 97.40%) as a white solid which was used in the next step without further purification. LC/MS (ESI) m/z: 259 (M+H)⁺.

### Step 3: tert-butyl (S)-8-(((4-cyanothiophen-2-yl)methyl)carbamoyl)-1,4-dioxa-7-azaspiro [4.4]nonane-7-carboxylate (7)

To a solution of 6 (0.5 g, 1.83 mmol, 1.0 equiv.) in DMF (5 mL) was added 5-(aminomethyl)thiophene-3-carbonitrile. HCl (5) (0.35 g, 2.01 mmol, 1.1 equiv.), HATU (0.83 g, 2.2 mmol, 1.2 equiv.) and DIPEA (1.1 mL, 5.49 mmol, 3.0 equiv.). The reaction was stirred at room temperature for 30 minutes. Water (5 mL) was added, and the liquid decanted. The residue was taken up in EtOAc, washed with brine, dried over Na₂SO₄, and concentrated. Purification by Combi Flash; 12 g column, solvent A=CH₂Cl₂, solvent B=MeOH, 100% A to 5% B gave 7 (0.62 g, 1.58 mmol, yield 86.34%) as a brown oil. LC/MS (ESI) m/z: 394 (M+H)⁺.

### Step 4: (S)—N-((4-cyanothiophen-2-yl)methyl)-1,4-dioxa-7-azaspiro[4.4]nonane-8-carboxamide (8)

TFA (2.23 mL) was added to a solution of 7 (0.62 g, 1.58 mmol, 1.0 equiv.) in CH₂Cl₂ (5 mL) at ice-bath temperature. The reaction was stirred at room temperature for 3 hours and then concentrated to dryness to give 8 (0.45 g, 1.53 mmol, yield 96.84%) as a light-yellow oil which was used in the next step without further purification. LC/MS (ESI) m/z: 294 (M+H)+.

### Step 5: (S)-7-((4-bromobenzoyl)glycyl)-N-((4-cyanothiophen-2-yl)methyl)-1,4-dioxa-7-azaspiro[4.4]nonane-8-carboxamide (9)

To a solution of 4 (0.15 g, 0.58 mmol, 1.0 equiv.) in DMF (2 mL) was added compound 8 (0.19 g, 0.64 mmol, 1.1 equiv.), HATU (0.29 g, 0.76 mmol, 1.3 equiv.) and DIPEA (0.30 mL, 1.74 mmol, 3.0 equiv.). The reaction was stirred at room temperature for 30 minutes. Water (5 mL) and EtOAc were added, and the two layers separated. The aqueous layer was extracted with EtOAc (5 mL×2), washed with brine, dried over Na₂SO₄, and concentrated. Purification by Combi Flash; 12 g column, solvent A=CH₂Cl₂, solvent B=MeOH, 100% A to 5% B gave 9 (0.22 g, 0.41 mmol, yield 70.69%) as an off yellow solid. LC/MS (ESI) m/z: 534 (M+H)⁺.

### Step 6: (S)—N-((4-cyanothiophen-2-yl)methyl)-7-((4-(1-methyl-1H-indol-5-yl)benzoyl)glycyl)-1,4-dioxa-7-azaspiro[4.4]nonane-6-carboxamide (10)

A mixture of (1-methylindol-5-yl) boronic acid (20 mg, 0.11 mmol, 1.2 equiv.), compound 9 (50 mg, 0.094 mmol, 1 equiv.), Na₂CO₃ (20 mg, 0.19 mmol, 2 equiv.), and Pd(dppf)Cl₂-DCM (8 mg, 0.009 mmol) in water (0.3 ml) and 1,4-dioxane (3 mL) was placed in a microwave reactor for 30 minutes at 110° C. The reaction was cooled to room temperature, diluted with water and EtOAc. The two layers were separated, and the aqueous layer was extracted with EtOAc (5 mL×2), washed with brine, dried over Na₂SO₄ and concentrated. Purification by Combi Flash; 4 g column, solvent A=CH₂Cl₂, solvent B=MeOH, 100% A to 5% B gave 10 (31 mg, 0.053 mmol, 56.38% yield). LC/MS (ESI) m/z: 584 (M+H)+.

### Step 7: (S)—N-((4-(N-hydroxycarbamimidoyl)thiophen-2-yl)methyl)-7-((4-(1-methyl-1H-indol-5-yl)benzoyl)glycyl)-1,4-dioxa-7-azaspiro[4.4]nonane-8-carboxamide (11)

To a solution of 10 (25 mg, 0.043 mmol, 1.0 equiv.) in EtOH (1 mL) was added Hydroxylamine hydrochloride (7.5 mg, 0.11 mmol, 2.5 equiv.), and DIPEA (22.4 μL, 0.13 mmol, 3 equiv.). The reaction was stirred at room temperature for 24 hours. The solvent was removed under Rota vamp evaporation. The residue was taken in CH₂Cl₂, washed with brine, dried over Na₂SO₄ and concentrated. Purification by combi Flash; 4 g column, solvent A=CH₂Cl₂, solvent B=MeOH. 100% A to 5% B gave 11 (15 mg, 0.024 mmol, 57% yield) as a brown oil. LC/MS (ESI) m/z: 617 (M+H)⁺.

### Step 8: (S)—N-((4-carbamimidoylthiophen-2-yl)methyl)-7-((4-(1-methyl-1H-indol-5-yl)benzo yl)glycyl)-1,4-dioxa-7-azaspiro[4.4]nonane-8-carboxamide (Compound 104)

To a solution of 11 (15 mg, 0.024 mmol, 1.0 equiv.) in MeOH (1 mL) was added Ra—Ni (50 mass %) in H₂O (4.2 mg, 0.0024 mmol, 0.1 equiv.), and AcOH (10 μL). The flask was evacuated and backfilled with H₂ in a balloon. The reaction was stirred at room temperature overnight and then filtered through Celite pad. The filtrate was concentrated under reduced pressure and then purified by HPLC to give Compound 104 (2.5 mg, 0.0042 mmol, 1.75% yield) as a white solid. LC/MS (ESI) m/z: 601 (M+H)⁺. ¹H NMR (400 MHz, DMSO-d₆) δ 8.68 (s, 1H), 8.62 (s, 1H), 8.47 (s, 1H), 8.32 (s, 1H), 8.10-7.89 (m, 2H), 7.79 (d, J=8.2 Hz, 1H), 7.54 (s, 1H), 7.48 (s, 1H), 7.37 (d, J=2.9 Hz, 1H), 6.51 (s, 1H), 5.17 (s, 1H), 4.71-4.57 (m, 2H), 4.50-4.40 (m, 2H), 4.43-4.33 (m, 4H), 4.31 (dd, J=16.6, 5.6 Hz, 1H), 4.02 (td, J=16.2, 5.6 Hz, 1H), 3.70 (s, 3H), 3.43 (dd, J=6.1, 2.4 Hz, 1H), 2.28 (t, J=12.3 Hz, 1H), 1.99 (dd, J=13.2, 3.1 Hz, 1H).

### Scheme 104: (S)—N-((4-carbamimidoylthiophen-2-yl)methyl)-7-((3-fluoro-5-phenylpicolinoyl)glycyl)-1,4-dioxa-7-azaspiro[4.4]nonane-8-carboxamide (Compound 105)

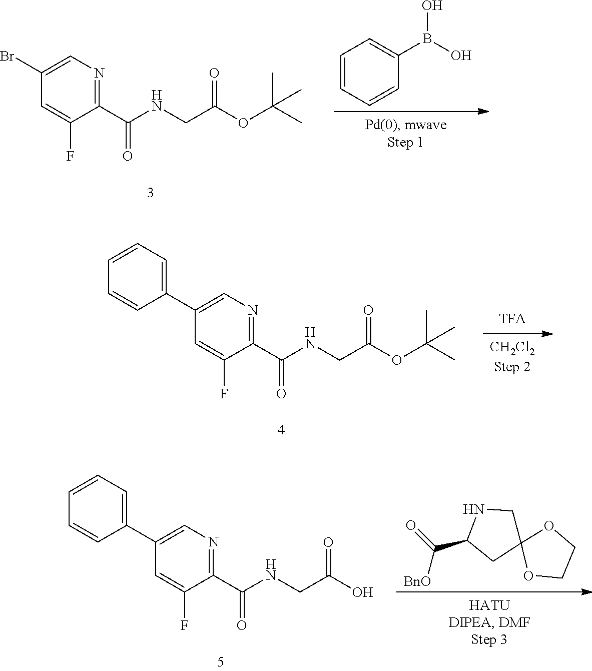

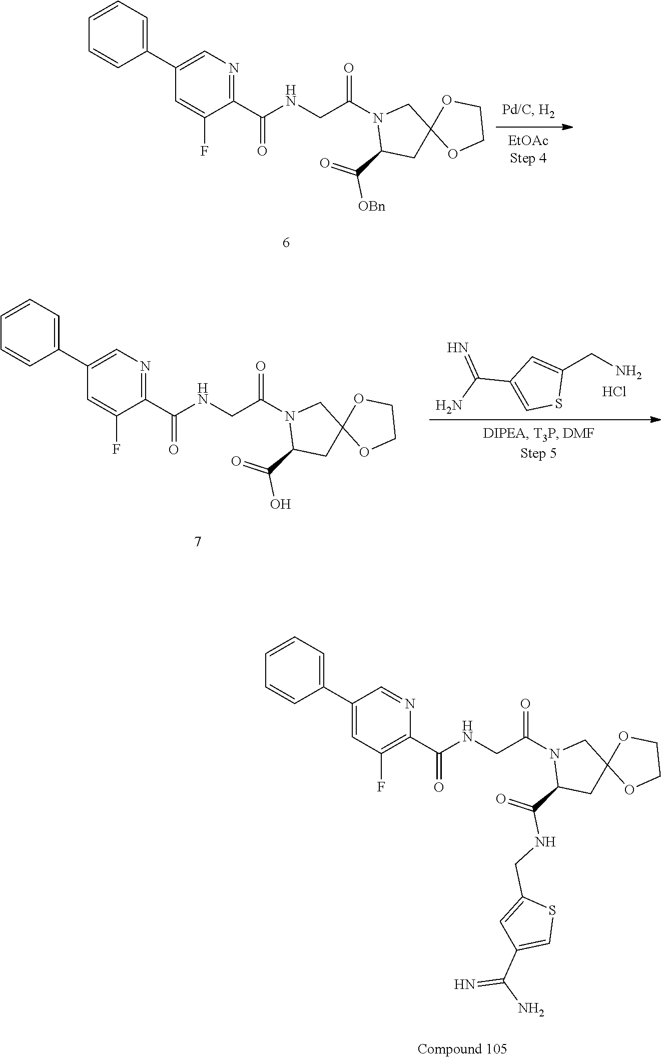 [SMILES: O=C(NCC(=O)N1CC2(C[C@H]1C(=O)OCc1ccccc1)OCCO2)c1ncc(-c2ccccc2)cc1F ; O=C(NCC(=O)N1CC2(C[C@H]1C(=O)O)OCCO2)c1ncc(-c2ccccc2)cc1F ; N=C(N)c1csc(CN)c1 ; Cl ; N=C(N)c1csc(CNC(=O)[C@@H]2CC3(CN2C(=O)CNC(=O)c2ncc(-c4ccccc4)cc2F)OCCO3)c1]

Compound 105 was prepared as a white solid from phenylboronic acid, tert-butyl (5-bromo-3-fluoropicolinoyl)glycinate, benzyl (8S)-1,4-dioxa-7-azaspiro[4.4]nonane-8-carboxylate, and 5-(aminomethyl)thiophene-3-carboximidamide. HCl based on the procedures set forth in Scheme 101. LC/MS (ESI) m/z: 567 (M+H)⁺. ¹H NMR (400 MHz, DMSO-d₆) δ 8.85 (s, 1H), 8.67 (s, 1H), 8.36-8.32 (m, 1H), 8.31 (s, 1H), 8.18 (d, J=12.2 Hz, 1H), 7.78 (d, J=7.5 Hz, 2H), 7.36 (d, J=7.5 Hz, 2H) 4.69-4.34 (m, 3H), 4.24-4.17 (m, 2H), 4.14 (dd, J=16.6, 5.6 Hz, 2H), 4.04-3.81 (m, 2H), 3.79-3.13 (m, 2H), 2.78-2.65 (m, 2H), 2.36-2.25 (m, 1H), 2.04 (dt, J=12.1, 6.0 Hz, 1H).

### Scheme 105: Synthesis of 18-(((S)-4-((2-(((3R,5S)-5-(((R)-1-(4-carbamimidoylthiophen-2-yl)ethyl)carbamoyl)-3-fluoro-1-((4-(4-fluorophenoxy)benzoyl)glycyl)pyrrolidin-3-yl)methoxy)ethyl)amino)-1-carboxy-4-oxobutyl)amino)-18-oxooctadecanoic acid (Compound

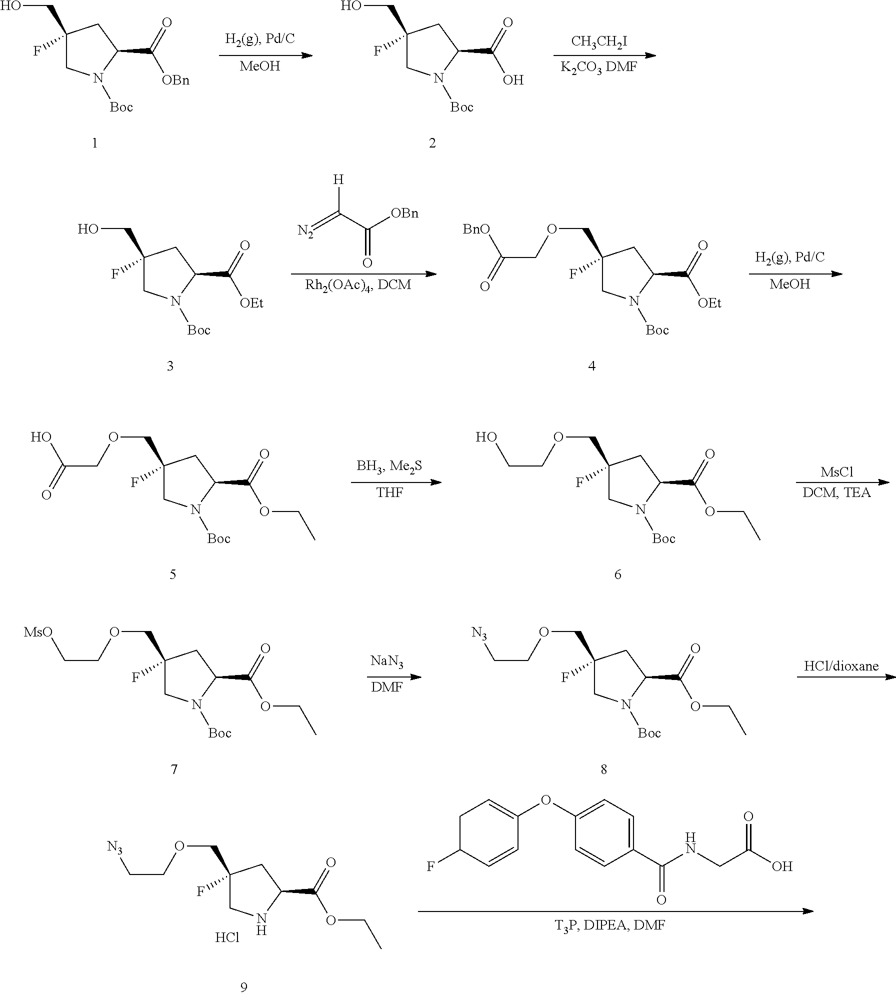

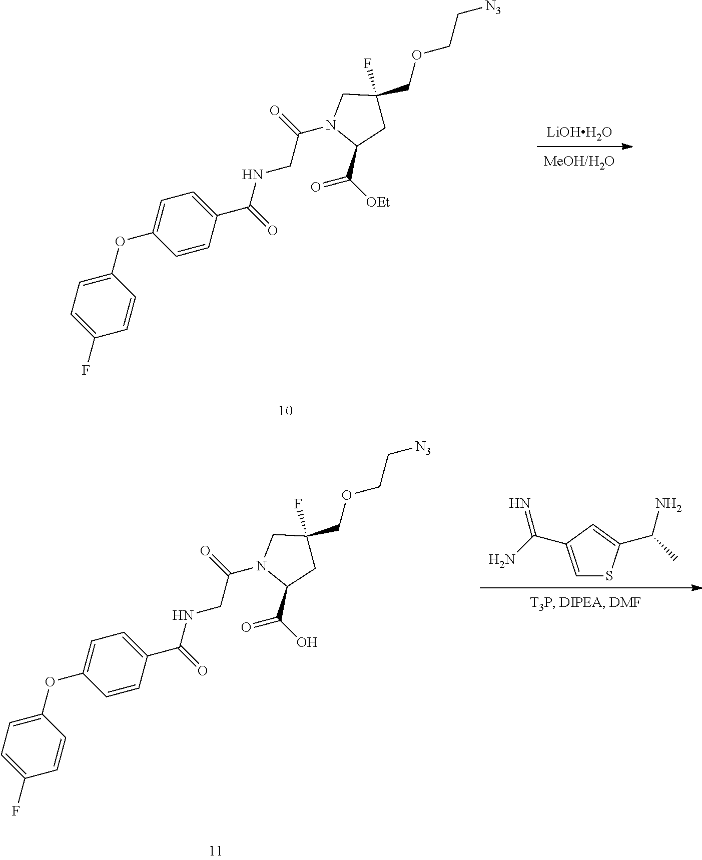 [SMILES: CCOC(=O)[C@@H]1C[C@](F)(COCCN=[N+]=[N-])CN1C(=O)CNC(=O)c1ccc(Oc2ccc(F)cc2)cc1 ; "LiOH·H2O" ; [N-]=[N+]=NCCOC[C@@]1(F)C[C@@H](C(=O)O)N(C(=O)CNC(=O)c2ccc(Oc3ccc(F)cc3)cc2)C1 ; C[C@@H](N)c1cc(C(=N)N)cs1]

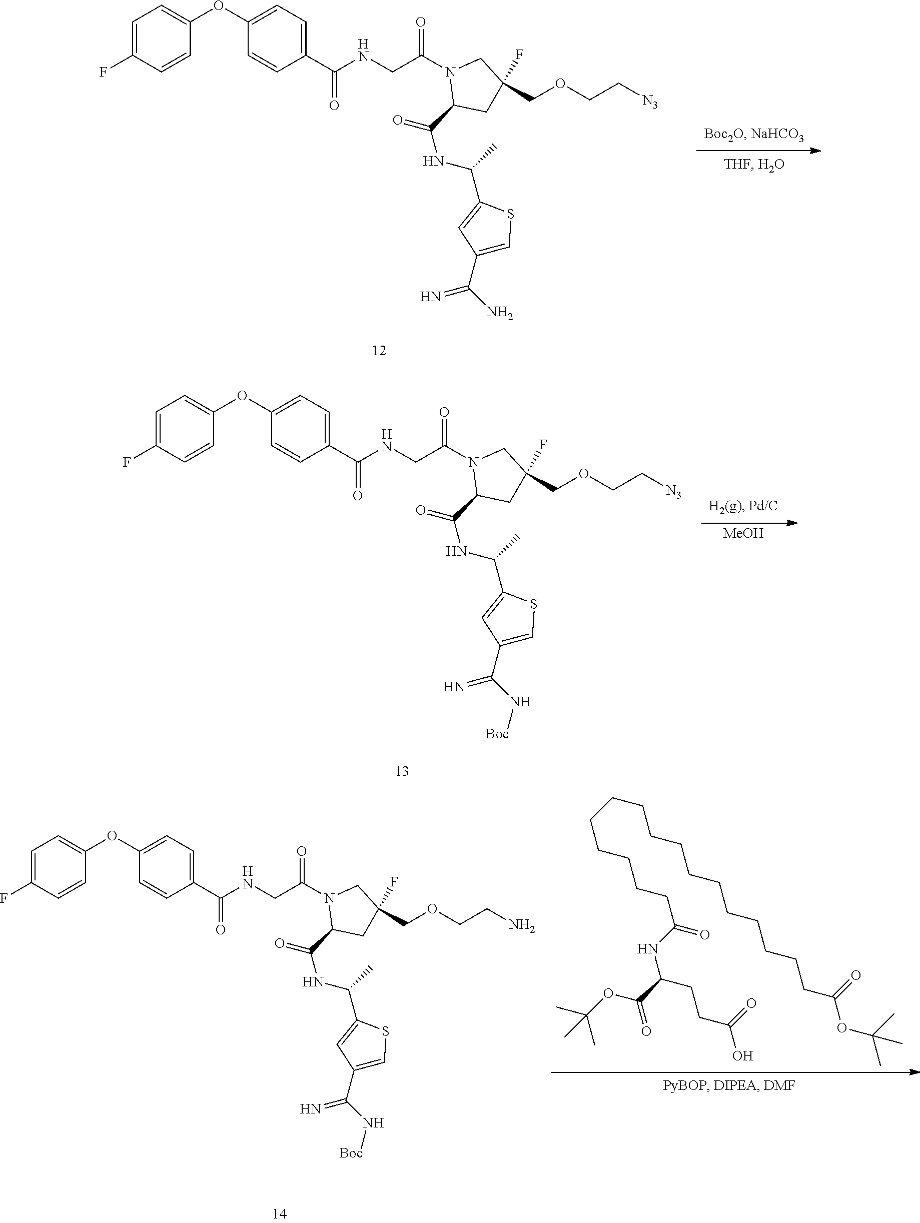

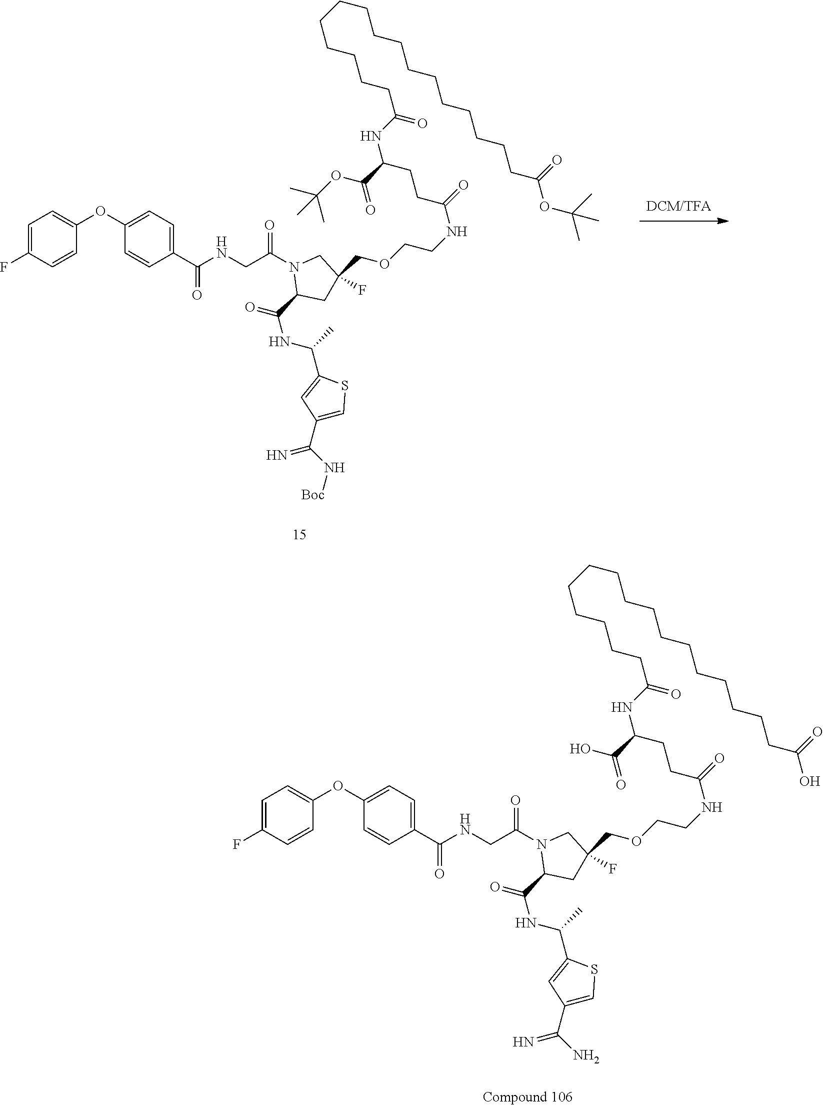 [SMILES: C[C@@H](NC(=O)[C@@H]1C[C@](F)(COCCNC(=O)CC[C@H](NC(=O)CCCCCCCCCCCCCCCCC(=O)OC(C)(C)C)C(=O)OC(C)(C)C)CN1C(=O)CNC(=O)c1ccc(Oc2ccc(F)cc2)cc1)c1cc(C(=N)NC(=O)OC(C)(C)C)cs1 ; C[C@@H](NC(=O)[C@@H]1C[C@](F)(COCCNC(=O)CC[C@H](NC(=O)CCCCCCCCCCCCCCCCC(=O)O)C(=O)O)CN1C(=O)CNC(=O)c1ccc(Oc2ccc(F)cc2)cc1)c1cc(C(=N)N)cs1]

### Step 1: (2S,4R)-1-(tert-butoxycarbonyl)-4-fluoro-4-(hydroxymethyl)pyrrolidine-2-carboxylic acid (2)

To a solution of 2-benzyl 1-(tert-butyl) (2S,4R)-4-fluoro-4-(hydroxymethyl)pyrrolidine-1,2-dicarboxylate (2.5 g, 7.10 mmol) in MeOH (25 mL) was added Pd/C (100 mg, 10% wt.), and the reaction mixture was stirred at 25° C. under H₂ atmosphere for 2 hours. The mixture was filtered, and concentrated to dryness under reduced pressure to give (2S,4R)-1-(tert-butoxycarbonyl)-4-fluoro-4-(hydroxymethyl)pyrrolidine-2-carboxylic acid (1.8 g, yield 86.7%) as a white solid, which was used in next step without purification. LC/MS (ESI) m/z: 264 (M+H)⁺.

### Step 2: 1-(tert-butyl) 2-ethyl (2S,4R)-4-fluoro-4-(hydroxymethyl)pyrrolidine-1,2-dicarboxylate (3)

To a mixture of (2S,4R)-1-(tert-butoxycarbonyl)-4-fluoro-4-(hydroxymethyl)pyrrolidine-2-carboxylic acid (1.8 g, 6.84 mmol) and CH₃CH₂I (1.6 g, 10.27 mmol) in DMF (30 mL) was added K₂CO₃ (2.83 g, 20.52 mmol) and the mixture was stirred at 28° C. for 16 hours. The mixture was diluted with EtOAc and washed with aq·NH₄Cl twice. The combined organic layers were dried over anhydrous Na₂SO₄, filtered, and concentrated under reduced pressure to dryness. The residue was purified by column chromatography on silica gel (PE:EtOAc=1:1) to give 1-(tert-butyl) 2-ethyl (2S,4R)-4-fluoro-4-(hydroxymethyl)pyrrolidine-1,2-dicarboxylate (1.8 g, yield 90.4%) as a yellow oil. LC/MS (ESI) m/z: 292 (M+H)⁺.

### Step 3: 1-(tert-butyl) 2-ethyl (2S,4R)-4-((2-(benzyloxy)-2-oxoethoxy)methyl)-4-fluoropyrrolidine-1,2-dicarboxylate (4)

To a mixture of 1-(tert-butyl) 2-ethyl (2S,4R)-4-fluoro-4-(hydroxymethyl)pyrrolidine-1,2-dicarboxylate (1.8 g, 6.18 mmol) and Rh₂(OAc)₄ (273 mg, 0.62 mmol) in DCM (20.0 mL) was added benzyl 2-diazoacetate (4.4 g, 24.72 mmol) in DCM (5.0 mL) at 0° C. under N₂ atmosphere and the mixture was stirred at 25° C. for 16 hours. The mixture was diluted with H₂O and extracted with EtOAc twice. The combined organic layers were dried over anhydrous Na₂SO₄, filtered, and concentrated to dryness under reduced pressure. The residue was purified by column chromatography on silica gel (PE:EtOAc=4:1) to give 1-(tert-butyl) 2-ethyl (2S,4R)-4-((2-(benzyloxy)-2-oxoethoxy)methyl)-4-fluoropyrrolidine-1,2-dicarboxylate (1.6 g, yield 59.2%) as a colorless oil. LC/MS (ESI) m/z: 440 (M+H)⁺.

### Step 4: 2-(((3R,5S)-1-(tert-butoxycarbonyl)-5-(ethoxycarbonyl)-3-fluoropyrrolidin-3-yl) methoxy)acetic acid (5)

To a solution of 1-(tert-butyl) 2-ethyl (2S,4R)-4-((2-(benzyloxy)-2-oxoethoxy) methyl)-4-fluoropyrrolidine-1,2-dicarboxylate (1.6 g, 3.64 mmol) in MeOH (20 mL) was added Pd/C (100 mg, 10% wt.), and the reaction mixture was stirred at 25° C. under H₂ atmosphere for 2 hours. The mixture was filtered, and concentrated to dryness under reduced pressure to give 2-(((3R,5S)-1-(tert-butoxycarbonyl)-5-(ethoxycarbonyl)-3-fluoropyrrolidin-3-yl)methoxy)acetic acid (1.22 g, yield 96.1%) as a colorless oil, which was used in next step without purification. LC/MS (ESI) m/z: 350 (M+H)⁺.

### Step 5: 1-(tert-butyl) 2-ethyl (2S,4R)-4-fluoro-4-((2-hydroxyethoxy)methyl)pyrrolidine-1,2-dicarboxylate (6)

To a solution of 2-(((3R,5S)-1-(tert-butoxycarbonyl)-5-(ethoxycarbonyl)-3-fluoropyrrolidin-3-yl)methoxy)acetic acid (1.22 g, 3.50 mmol) in THF (15.0 mL) was added BH₃·Me₂S (1.0 mL, 10.50 mmol) slowly at 0° C. under N₂ atmosphere and the mixture was stirred at 25° C. for 1 hour. The mixture was quenched with MeOH and diluted with EtOAc and washed with brine, dried over anhydrous Na₂SO₄, filtered, and concentrated to dryness under reduced pressure. The residue was purified by chromatography on silica gel (PE:EtOAc=1:1) to give 1-(tert-butyl) 2-ethyl (2S,4R)-4-fluoro-4-((2-hydroxyethoxy)methyl)pyrrolidine-1,2-dicarboxylate (920 mg, yield 78.6%) as a colorless oil. LC/MS (ESI) m/z: 336 (M+H)⁺.

### Step 6: 1-(tert-butyl) 2-ethyl (2S,4R)-4-fluoro-4-((2-((methylsulfonyl)oxy)ethoxy)methy) pyrrolidine-1,2-dicarboxylate (7)

To a mixture of 1-(tert-butyl) 2-ethyl (2S,4R)-4-fluoro-4-((2-hydroxyethoxy)methyl) pyrrolidine-1,2-dicarboxylate (920 mg, 2.75 mmol) and TEA (3.8 mL, 27.46 mmol) in DCM (10.0 mL) was added MsCl (1.1 mL, 13.75 mmol) slowly at 0° C. under N₂ atmosphere and the mixture was stirred at 0° C. for 1 hour. The mixture was diluted with DCM and washed with water, dried over anhydrous Na₂SO₄, filtered, and concentrated to dryness under reduced pressure to give 1-(tert-butyl) 2-ethyl (2S,4R)-4-fluoro-4-((2-((methylsulfonyl)oxy)ethoxy) methyl)pyrrolidine-1,2-dicarboxylate (1.1 g, yield 96.5%) as a brown oil, which was used directly in next step without further purification. LC/MS (ESI) m/z: 414 (M+H)⁺.

### Step 7: 1-(tert-butyl) 2-ethyl (2S,4R)-4-((2-azidoethoxy)methyl)-4-fluoropyrrolidine-1,2-dicarboxylate (8)

To a solution of 1-(tert-butyl) 2-ethyl (2S,4R)-4-fluoro-4-((2-((methylsulfonyl)oxy)ethoxy) methyl)pyrrolidine-1,2-dicarboxylate (1.5 g, 3.63 mmol) in DMF (20.0 mL) was added NaN₃ (600 mg, 9.08 mmol) and the mixture was stirred at 40° C. for 16 hours. The mixture was diluted with DCM and washed with brine, dried over anhydrous Na₂SO₄, filtered, and concentrated to dryness under reduced pressure. The residue was purified by chromatography on silica gel (PE:EtOAc=3:1) to give 1-(tert-butyl) 2-ethyl (2S,4R)-4-((2-azidoethoxy)methyl)-4-fluoropyrrolidine-1,2-dicarboxylate (800 mg, yield 61.5%) as a colorless oil. LC/MS (ESI) m/z: 361 (M+H)⁺.

### Step 8: ethyl (2S,4R)-4-((2-azidoethoxy)methyl)-4-fluoropyrrolidine-2-carboxylate hydrochloride (9)

A solution of 1-(tert-butyl) 2-ethyl (2S,4R)-4-((2-azidoethoxy)methyl)-4-fluoropyrrolidine-1,2-dicarboxylate (500 mg, 1.39 mmol) in HCl/1,4-dioxane (5 mL) was stirred at room temperature for 1 hour. The mixture was concentrated to dryness under reduced pressure to give ethyl (2S,4R)-4-((2-azidoethoxy)methyl)-4-fluoropyrrolidine-2-carboxylate hydrochloride (360 mg, yield 99.7%) as a colorless oil which was directly used in the next reaction without further purification. LC/MS (ESI) m/z: 261 (M+H)⁺.

### Step 9: ethyl (2S,4R)-4-((2-azidoethoxy)methyl)-4-fluoro-1-((4-(4-fluorophenoxy)benzoyl) glycyl)pyrrolidine-2-carboxylate (10)

To a mixture of (4-(4-fluorophenoxy)benzoyl)glycine (481 mg, 1.66 mmol) and ethyl (2S,4R)-4-((2-azidoethoxy)methyl)-4-fluoropyrrolidine-2-carboxylate HCl salt (361 mg, 1.38 mmol) in DMF (10 mL) was added DIPEA (1.10 mL, 6.90 mmol) and T₃P (2.6 g, 4.14 mmol, 50% wt. in EtOAc) under N₂ atmosphere and the reaction mixture was stirred at room temperature for 2 hours. The mixture was diluted with EtOAc, washed with saturated aq·NaHCO₃ and brine, dried over anhydrous Na—SO₄, filtered, and concentrated under reduced pressure to dryness. The residue was purified by column chromatography on silica gel (PE:EtOAc=1:2) to give ethyl (2S,4R)-4-((2-azidoethoxy)methyl)-4-fluoro-1-((4-(4-fluorophenoxy)benzoyl)glycyl)pyrrolidine-2-carboxylate (356 mg, yield 48.3%) as a brown oil. LC/MS (ESI) m/z: 532 (M+H)⁺.

### Step 10: (2S,4R)-4-((2-azidoethoxy)methyl)-4-fluoro-1-((4-(4-fluorophenoxy)benzoyl)glycyl) pyrrolidine-2-carboxylic acid (11)

To a solution of ethyl (2S,4R)-4-((2-azidoethoxy)methyl)-4-fluoro-1-((4-(4-fluorophenoxy) benzoyl)glycyl)pyrrolidine-2-carboxylate (356 mg, 0.67 mol) in MeOH (3 mL) and water (1 mL) was added LiOH·H₂O (42 mg, 1.01 mmol) at 0° C. and the mixture was stirred at room temperature for 2 hours. The mixture was acidified with 1 N aq·HCl to pH˜3 and extracted with EtOAc twice. The combined organic layers were washed with brine, dried over anhydrous Na₂SO₄, filtered, and concentrated under reduced pressure to give (2S,4R)-4-((2-azidoethoxy)methyl)-4-fluoro-1-((4-(4-fluorophenoxy)benzoyl)glycyl)pyrrolidine-2-carboxylic acid (300 mg, yield 89.0%) as a yellow solid, which was used directly in the next reaction. LC/MS (ESI) (m/z): 504 (M+H)⁺.

### Step 11: (2S,4R)-4-((2-azidoethoxy)methyl)-N—((R)-1-(4-carbamimidoylthiophen-2-yl)ethyl)-4-fluoro-1-((4-(4-fluorophenoxy)benzoyl)glycyl)pyrrolidine-2-carboxamide (12)

To a mixture of (2S,4R)-4-((2-azidoethoxy)methyl)-4-fluoro-1-((4-(4-fluorophenoxy)benzoyl) glycyl)pyrrolidine-2-carboxylic acid (300 mg, 0.60 mmol) and (R)-5-(1-aminoethyl)thiophene-3-carboximidamide HCl salt (304 mg, 1.80 mmol) in DMF (5 mL) was added DIPEA (0.60 mL, 3.60 mmol) and T₃P (1.14 g, 1.80 mmol, 50% wt. in EtOAc) under N₂ atmosphere and the reaction mixture was stirred at room temperature for 2 hours. The mixture was diluted with EtOAc, washed with saturated aq·NaHCO₃ and brine, dried over anhydrous Na₂SO₄, filtered, and concentrated under reduced pressure to give (2S,4R)-4-((2-azidoethoxy)methyl)-N—((R)-1-(4-carbamimidoylthiophen-2-yl)ethyl)-4-fluoro-1-((4-(4-fluorophenoxy)benzoyl)glycyl)pyrrolidine-2-carboxamide (385 mg, yield 98.7%) as a brown oil, which was used directly in the next reaction. LC/MS (ESI) m/z: 655 (M+H)⁺.

### Step 12: tert-butyl ((5-((R)-1-((2S,4R)-4-((2-azidoethoxy)methyl)-4-fluoro-1-((4-(4-fluorophenoxy)benzoyl)glycyl)pyrrolidine-2-carboxamido)ethyl)thiophen-3-yl)(imino)methyl)carbamate (13)

To a mixture of (2S,4R)-4-((2-azidoethoxy)methyl)-N—((R)-1-(4-carbamimidoylthiophen-2-yl) ethyl)-4-fluoro-1-((4-(4-fluorophenoxy)benzoyl)glycyl)pyrrolidine-2-carboxamide (626 mg, 0.96 mmol) and di-tert-butyl dicarbonate (626 mg, 2.88 mmol) in THF (6 mL) was added a solution of NaHCO₃ (804 g, 9.60 mmol) in H₂O (6 mL) at 0° C. and the mixture was stirred at 25° C. for 16 hours. The mixture was diluted with water and extracted with EtOAc twice. The combined organic layers were dried over anhydrous Na₂SO₄, filtered, and concentrated to dryness under reduced pressure. The residue was purified by flash chromatography (DCM:MeOH=20:1) to give tert-butyl ((5-((R)-1-((2S,4R)-4-((2-azidoethoxy) methyl)-4-fluoro-1-((4-(4-fluorophenoxy)benzoyl)glycyl)pyrrolidine-2-carboxamido)ethyl)thiophen-3-yl)(imino)methyl)carbamate (160 mg, yield 22.2%) as a white solid. LC/MS (ESI) m/z: 755(M+H)⁴.

### Step 13: tert-butyl ((5-((R)-1-((2S,4R)-4-((2-aminoethoxy)methyl)-4-fluoro-1-((4-(4-fluorophenoxy)benzoyl)glycyl)pyrrolidine-2-carboxamido)ethyl)thiophen-3-yl)(imino)methyl)carbamate (14)

To a solution of tert-butyl ((5-((R)-1-((2S,4R)-4-((2-azidoethoxy)methyl)-4-fluoro-1-((4-(4-fluorophenoxy)benzoyl)glycyl)pyrrolidine-2-carboxamido)ethyl)thiophen-3-yl)(imino)methyl)carbamate (160 mg, 0.21 mmol) in MeOH (5 mL) was added Pd/C (20 mg, 10% wt.), and the reaction mixture was stirred at 25° C. under H₂ atmosphere for 2 hours. The mixture was filtered, and concentrated to dryness under reduced pressure to give tert-butyl ((5-((R)-1-((2S,4R)-4-((2-aminoethoxy)methyl)-4-fluoro-1-((4-(4-fluorophenoxy)benzoyl)glycyl)pyrrolidine-2-carboxamido)ethyl)thiophen-3-yl)(imino)methyl)carbamate (145 mg, yield 94.2%) as a brown solid, which was used directly in the next reaction.LC/MS (ESI) m/z: 729 (M+H)⁺.

### Step 14: tert-butyl 18-(((S)-1-(tert-butoxy)-5-((2-(((3R,5S)-5-(((R)-1-(4-(N-(tert-butoxycarbonyl)carbamimidoyl)thiophen-2-yl)ethyl)carbamoyl)-3-fluoro-1-((4-(4-fluorophenoxy)benzoyl)glycyl)pyrrolidin-3-yl)methoxy)ethyl)amino)-1,5-dioxopentan-2-yl)amino)-18-oxooctadecanoate (15)

To a mixture of tert-butyl ((5-((R)-1-((2S,4R)-4-((2-aminoethoxy)methyl)-4-fluoro-1-((4-(4-fluorophenoxy)benzoyl)glycyl)pyrrolidine-2-carboxamido)ethyl)thiophen-3-yl)(imino)methyl)carbamate (50 mg, 0.07 mmol) and PyBOP (54 mg, 0.11 mmol) in DMF (3 mL) was added DIPEA (0.07 mL, 0.42 mmol) and (S)-5-(tert-butoxy)-4-(18-(tert-butoxy)-18-oxooctadecanamido)-5-oxopentanoic acid (456 mg, 0.84 mmol) in batches under N₂ atmosphere and the reaction mixture was stirred at room temperature for 2 hours. The mixture was concentrated to dryness under reduced pressure. The residue was purified by column chromatography on silica gel (DCM:MeOH=20:1) to give tert-butyl 18-(((S)-1-(tert-butoxy)-5-((2-(((3R,5S)-5-(((R)-1-(4-(N-(tert-butoxycarbonyl)carbamimidoyl)thiophen-2-yl)ethyl)carbamoyl)-3-fluoro-1-((4-(4-fluorophenoxy)benzoyl)glycyl)pyrrolidin-3-yl)methoxy)ethyl)amino)-1,5-dioxopentan-2-yl)amino)-18-oxooctadecanoate (77 mg, yield 88.5%) as a brown oil. LC/MS (ESI) m/z: 1267 (M+H)⁺.

### Step 15: 18-(((S)-4-((2-(((3R,5S)-5-(((R)-1-(4-carbamimidoylthiophen-2-yl)ethyl)carbamoyl)-3-fluoro-1-((4-(4-fluorophenoxy)benzoyl)glycyl)pyrrolidin-3-yl)methoxy)ethyl)amino)-1-carboxy-4-oxobutyl)amino)-18-oxooctadecanoic acid (Compound 106)

To a solution of tert-butyl 18-(((S)-1-(tert-butoxy)-5-((2-(((3R,5S)-5-(((R)-1-(4-(N-(tert-butoxycarbonyl)carbamimidoyl)thiophen-2-yl)ethyl)carbamoyl)-3-fluoro-1-((4-(4-fluorophenoxy)benzoyl)glycyl)pyrrolidin-3-yl)methoxy)ethyl)amino)-1,5-dioxopentan-2-yl)amino)-18-oxooctadecanoate (77 mg, 0.06 mmol) in DCM (2 mL) was added TFA (1 mL) and the mixture was stirred at room temperature for 1 hour. The reaction mixture was concentrated to dryness under reduced pressure, co-evaporated with DCM and dried under vacuum. The residue was purified by prep-HPLC to give Compound 106 (15 mg, yield 23.4%) as a white solid. ¹H NMR (400 MHz, CD₃OD) δ 8.24 (t, J=3.9 Hz, 1H), 7.86 (d, J=8.8 Hz, 2H), 7.57 (d, J=17.3 Hz, 1H), 7.16 (t, J=8.6 Hz, 2H), 7.08 (dd, J=9.1, 4.5 Hz, 2H), 6.99 (d, J=8.8 Hz, 2H), 5.30 (dq, J=13.7, 6.8 Hz, 1H), 4.62 (t, J=8.7 Hz, 1H), 4.35-4.23 (m, 2H), 4.17-4.01 (m, 2H), 3.92 (dd, J=33.5, 12.2 Hz, 1H), 3.80-3.71 (m, 2H), 3.61 (t, J=5.2 Hz, 2H), 3.37 (d, J=3.7 Hz, 2H), 2.54 (dd, J=27.9, 11.4 Hz, 1H), 2.29-2.10 (m, 8H), 2.00-1.90 (m, 1H), 1.58 (d, J=6.9 Hz, 6H), 1.27 (m, 25H). LC/MS (ESI) (m/z): 1055 (M+H)⁺.

### Scheme 106: Synthesis of (S)—N-((4-carbamimidoylthiophen-2-yl)methyl)-7-((2′,4′-difluoro-[1,1′-biphenyl]-4-carbonyl)glycyl)-1,4-dioxa-7-azaspiro[4.4]nonane-8-carboxamide (Compound 107)

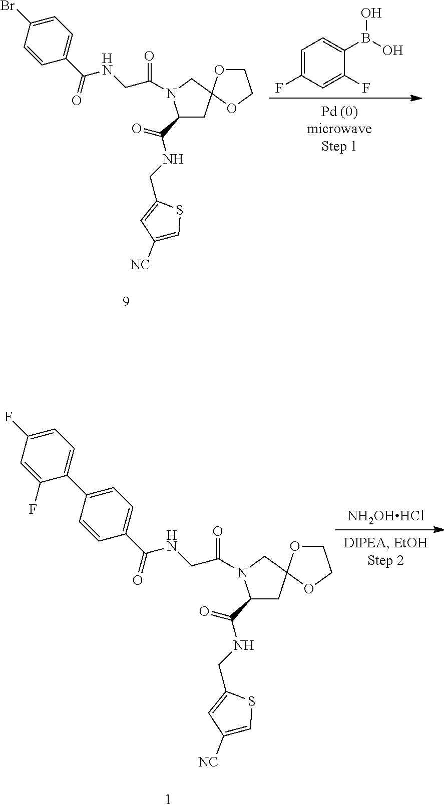

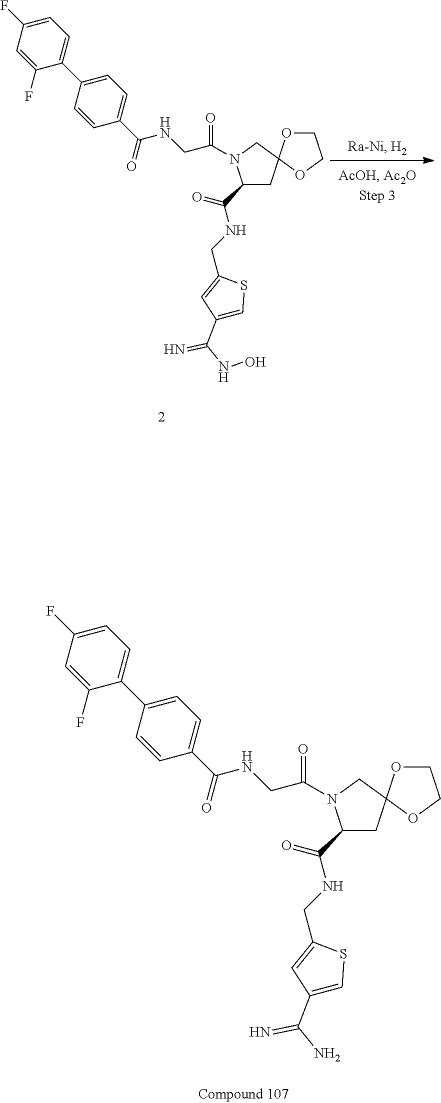 [SMILES: N=C(NO)c1csc(CNC(=O)[C@@H]2CC3(CN2C(=O)CNC(=O)c2ccc(-c4ccc(F)cc4F)cc2)OCCO3)c1 ; N=C(N)c1csc(CNC(=O)[C@@H]2CC3(CN2C(=O)CNC(=O)c2ccc(-c4ccc(F)cc4F)cc2)OCCO3)c1]

Compound 107 was prepared as a white solid from (S)-7-((4-bromobenzoyl)glycyl)-N-((4-cyanothiophen-2-yl)methyl)-1,4-dioxa-7-azaspiro[4.4]nonane-8-carboxamide, (2,4-difluorophenyl)boronic acid, and hydroxylamine hydrochloride based on the procedures set forth in Scheme 103. LC/MS (ESI) m/z: 584 (M+H)⁺. ¹H NMR (400 MHz, DMSO-d₆) δ 9.00 (s, 1H), 8.77 (t, J=5.7 Hz, 1H), 8.62 (t, J=5.8 Hz, 1H), 8.34 (d, J=13.5 Hz, 1H), 7.97 (d, J=8.0 Hz, 2H), 7.69 (d, J=7.8 Hz, 2H), 7.56-7.41 (m, 3H), 7.31 (tt, J=8.3, 3.5 Hz, 1H), 5.17 (s, 2H), 4.71-4.57 (m, 2H), 4.50-4.40 (m, 3H), 4.43-4.33 (m, 3H), 4.31 (dd, J=16.6, 5.6 Hz, 2H), 4.02 (td, J=16.2, 5.6 Hz, 1H), 3.43 (dd, J=6.1, 2.4 Hz, 1H), 2.28 (t, J=12.3 Hz, 1H), 2.00 (dd, J=13.2, 3.1 Hz, 1H).

### Scheme 107. (S)—N-((4-carbamimidoylthiophen-2-yl)methyl)-7-((4′-methyl-[1,1′-biphenyl]-4-carbonyl)glycyl)-1,4-dioxa-7-azaspiro[4.4]nonane-8-carboxamide (Compound 108)

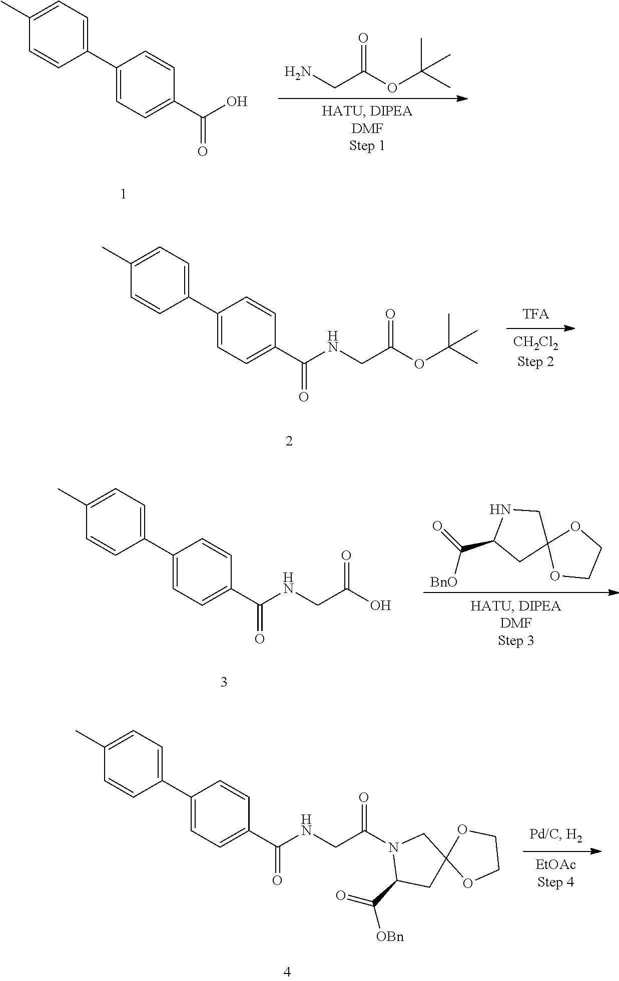

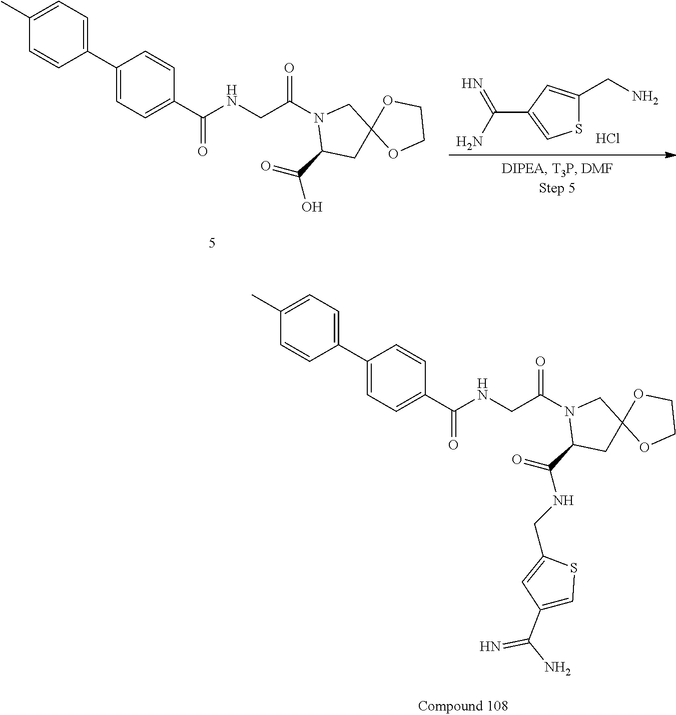 [SMILES: Cc1ccc(-c2ccc(C(=O)NCC(=O)N3CC4(C[C@H]3C(=O)O)OCCO4)cc2)cc1 ; N=C(N)c1csc(CN)c1 ; Cl ; Cc1ccc(-c2ccc(C(=O)NCC(=O)N3CC4(C[C@H]3C(=O)NCc3cc(C(=N)N)cs3)OCCO4)cc2)cc1]

Compound 108 was prepared as a white solid from 4-(p-tolyl)benzoic acid, 4′-methyl-[1,1′-biphenyl]-4-carboxylic acid, benzyl (8S)-1,4-dioxa-7-azaspiro[4.4]nonane-8-carboxylate, and 5-(aminomethyl)thiophene-3-carboximidamide. HCl based on the procedures set forth in Scheme 1. LC/MS (ESI) m/z: 562 (M+H)⁺. ¹H NMR (400 MHz, DMSO-d₆) δ 9.05-8.81 (m, 1H), 8.70 (t, J=5.7 Hz, 1H), 8.60 (s, 1H), 8.35 (d, J=24.0 Hz, 3H), 7.95 (dd, J=12.3, 8.0 Hz, 3H), 7.75 (d, J=8.0 Hz, 2H), 7.64 (d, J=7.8 Hz, 2H), 7.50 (d, J=17.8 Hz, 2H), 7.30 (d, J=7.9 Hz, 2H), 4.69-4.34 (m, 3H), 4.17 (dd, J=16.6, 5.6 Hz, 1H), 4.04-3.81 (m, 2H), 3.79-3.13 (m, 2H), 2.78-2.65 (m, 1H), 2.36 (s, 3H), 2.34-2.22 (m, 1H), 2.23-1.79 (m, 1H).

### Scheme 108: Synthesis of (S)-7-(([1,1′-biphenyl]-3-carbonyl)glycyl)-N—((R)-1-(4-carbamimidoylthiophen-2-yl)ethyl)-1,4-dioxa-7-azaspiro[4.4]nonane-8-carboxamide (Compound 109)

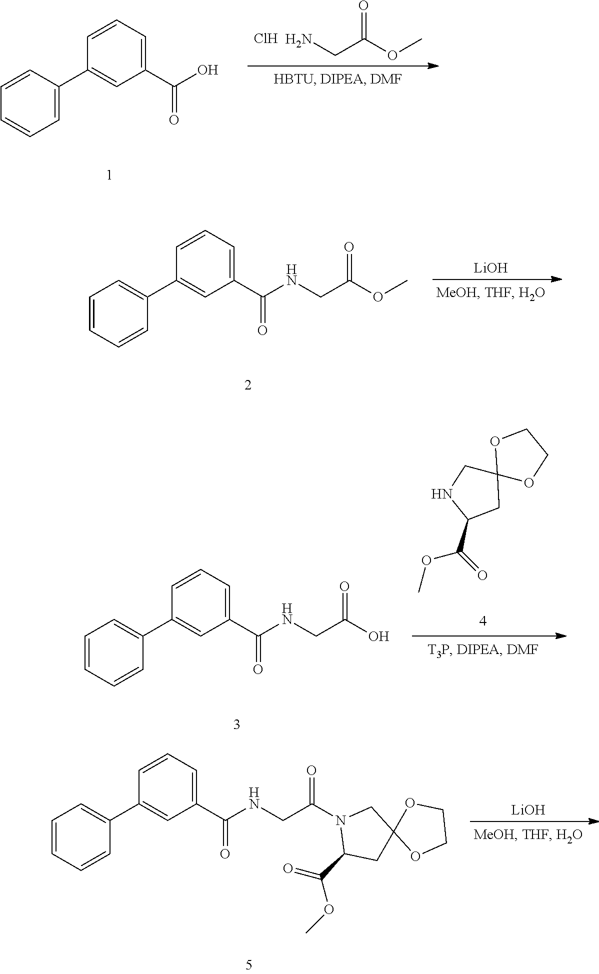

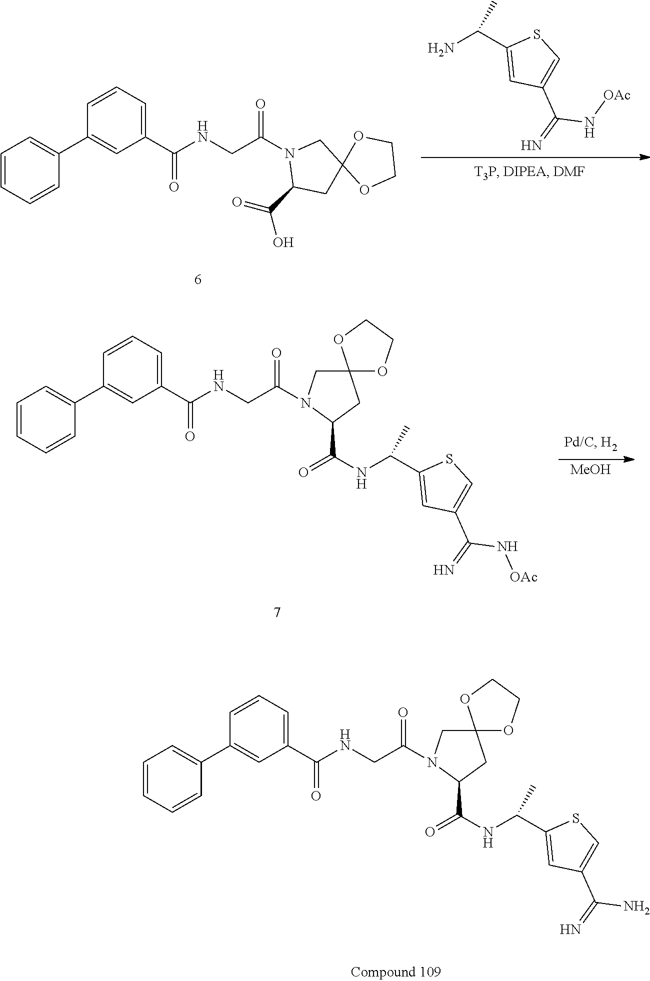

Compound 109 was prepared from [1,1′-biphenyl]-3-carboxylic acid, methyl glycinate, methyl (S)-1,4-dioxa-7-azaspiro[4.4]nonane-8-carboxylate, and (R)—N-acetoxy-5-(1-aminoethyl) thiophene-3-carboximidamide based on the procedures set forth in Scheme 89. ¹H NMR (400 MHz, CD₃OD) δ 8.56 (s, 1H), 8.15 (d, J=18.4 Hz, 2H), 7.83 (d, J=7.7 Hz, 2H), 7.67 (d, J=7.2 Hz, 2H), 7.58-7.50 (m, 2H), 7.47 (t, J=7.5 Hz, 2H), 7.38 (t, J=7.4 Hz, 1H), 5.31 (t, J=16.8 Hz, 1H), 4.61-4.55 (m, 1H), 4.02-3.94 (m, 4H), 3.85-3.77 (m, 2H), 2.44 (dd, J=13.1, 9.0 Hz, 1H), 2.22 (dd, J=12.9, 6.5 Hz, 1H), 1.61 (t, J=18.6 Hz, 3H), 1.29 (s, 1H), 1.21-1.09 (m, 1H). LC/MS (ESI) m/z: 562 (M+H)⁺.

### Scheme 109: Synthesis of (8S)—N-((4-carbamimidoylthiophen-2-yl)methyl)-7-((9-methyl-9H-fluorene-3-carbonyl)glycyl)-1,4-dioxa-7-azaspiro[4.4]nonane-8-carboxamide (Compound 110)

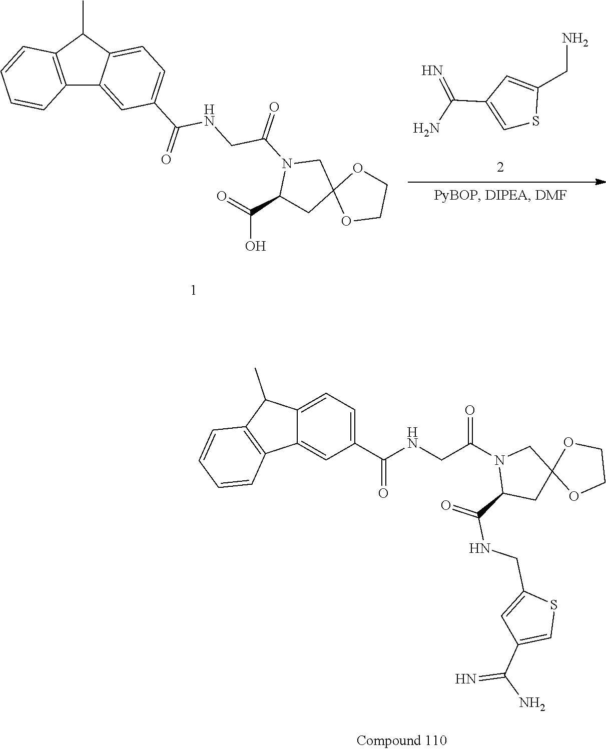 [SMILES: CC1c2ccccc2-c2cc(C(=O)NCC(=O)N3CC4(C[C@H]3C(=O)O)OCCO4)ccc21 ; N=C(N)c1csc(CN)c1 ; CC1c2ccccc2-c2cc(C(=O)NCC(=O)N3CC4(C[C@H]3C(=O)NCc3cc(C(=N)N)cs3)OCCO4)ccc21]

Compound 110 was prepared as a white solid from (8S)-7-{2-[(9-methyl-9H-fluoren-3-yl)formamido]acetyl}-1,4-dioxa-7-azaspiro[4.4]nonane-8-carboxylic acid and 5-(aminomethyl)thiophene-3-carboximidamide based on the procedures set forth in Scheme 3. ¹H NMR (400 MHz, CD₃OD) δ 8.53 (s, 1H), 8.33-8.13 (m, 2H), 7.82 (t, J=8.1 Hz, 2H), 7.63 (d, J=7.8 Hz, 1H), 7.57 (d, J=6.9 Hz, 1H), 7.48-7.32 (m, 3H), 4.66-4.55 (m, 3H), 4.21 (dd, J=47.4, 16.6 Hz, 2H), 4.00 (d, J=1.9 Hz, 4H), 3.79 (d, J=28.8 Hz, 2H), 3.18 (dt, J=19.5, 9.7 Hz, 1H), 2.45 (dd, J=13.1, 9.3 Hz, 1H), 2.24 (dd, J=13.3, 5.5 Hz, 1H), 1.53 (d, J=7.4 Hz, 3H). LC/MS (ESI) m/z: 574 (M+H)⁺.

### Scheme 110: Synthesis of (S)-7-((9H-fluorene-3-carbonyl)glycyl)-N—((R)-1-(4-carbamimidoylthiophen-2-yl)ethyl)-1,4-dioxa-7-azaspiro[4.4]nonane-8-carboxamide (Compound 111)

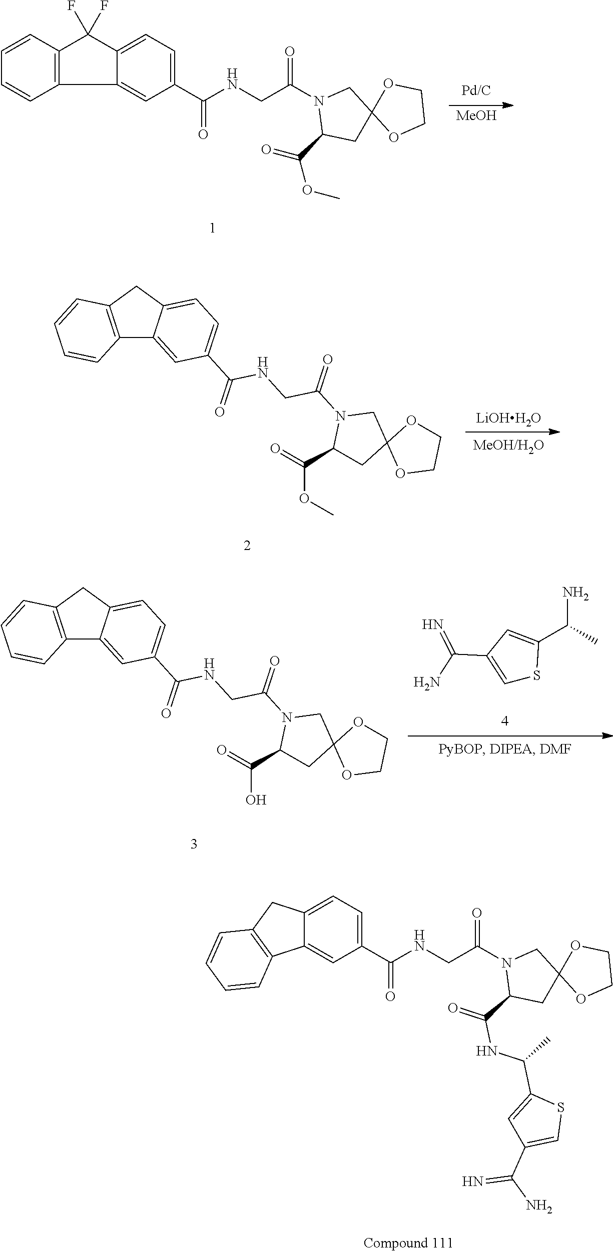

### Step 1: methyl (S)-7-((9H-fluorene-3-carbonyl)glycyl)-1,4-dioxa-7-azaspiro[4.4]nonane-8-carboxylate (2)

To a solution of methyl (S)-7-((9,9-difluoro-9H-fluorene-3-carbonyl)glycyl)-1,4-dioxa-7-azaspiro[4.4]nonane-8-carboxylate (150 mg, 0.32 mmol) in MeOH (3 mL) was added Pd/C (30 mg, 10% wt.) at room temperature and the mixture was degassed under N₂ atmosphere for three times and stirred under a H₂ balloon at room temperature overnight. The mixture was filtered, and the filtrate was concentrated to dryness under reduced pressure to give methyl (S)-7-((9H-fluorene-3-carbonyl)glycyl)-1,4-dioxa-7-azaspiro[4.4]nonane-8-carboxylate (120 mg, yield 86.6%) as a colorless oil, which was used directly in the next step. LC/MS (ESI) (m/z): 437 (M+H)⁺.

### Step 2: (S)-7-((9H-fluorene-3-carbonyl)glycyl)-1,4-dioxa-7-azaspiro[4.4]nonane-6-carboxylic acid (3)

To a solution of methyl (S)-7-((9H-fluorene-3-carbonyl)glycyl)-1,4-dioxa-7-azaspiro [4.4]nonane-8-carboxylate (110 mg, 0.25 mmol) in MeOH (2 mL) and water (1 mL) was added LiOH·H₂O (32 mg, 0.75 mmol) at 0° C. and the mixture was stirred at room temperature for 4 hours. The mixture was acidified with 1 N aq·HCl to pH˜3, extracted with EtOAc twice. The combined organic layers were washed with brine, dried over anhydrous Na₂SO₄, filtered, and concentrated under reduced pressure to dryness to give (S)-7-((9H-fluorene-3-carbonyl)glycyl)-1,4-dioxa-7-azaspiro[4.4]nonane-8-carboxylic acid (95 mg, yield 89.2%) as a colorless solid, which was used directly in the next step. LC/MS (ESI) (m/z): 423 (M+H)⁺.

### Step 3: (S)-7-((9H-fluorene-3-carbonyl)glycyl)-N—((R)-1-(4-carbamimidoylthiophen-2-yl)ethyl)-1,4-dioxa-7-azaspiro[4.4]nonane-8-carboxamide (Compound 112)

To a mixture of (S)-7-((9H-fluorene-3-carbonyl)glycyl)-1,4-dioxa-7-azaspiro[4.4]nonane-8-carboxylic acid (35 mg, 0.08 mmol) and (R)-5-(1-aminoethyl)thiophene-3-carboximidamide hydrochloride (34 mg, 0.16 mmol) in DMF (0.5 mL) was added DIPEA (31 mg, 0.24 mmol) and PyBOP (46 mg, 0.09 mmol) at 0° C. under N₂ atmosphere and the mixture was stirred at 25° C. for 2 hours. The mixture was diluted with water, extracted with EtOAc twice. The combined organic layers were dried over anhydrous Na₂SO₄, filtered, and concentrated to dryness under reduced pressure. The residue was purified by column chromatography on silica gel (DCM:MeOH=8:1) and prep-HPLC to give Compound 111 (4.2 mg, yield 8.8%) as a white solid. ¹H NMR (400 MHz, CD₃OD) δ 8.54 (s, 1H), 8.31 (s, 1H), 8.20 (s, 1H), 7.82 (dd, J=15.9, 7.7 Hz, 2H), 7.64 (d, J=7.9 Hz, 1H), 7.58 (d, J=7.3 Hz, 1H), 7.53 (d, J=9.9 Hz, 1H), 7.37 (dt, J=21.6, 7.3 Hz, 2H), 5.38-5.25 (m, 1H), 4.59 (s, 1H), 4.20 (d, J=1.8 Hz, 2H), 4.00 (dd, J=9.4, 4.9 Hz, 4H), 3.95 (s, 2H), 3.85-3.76 (m, 2H), 2.44 (dd, J=12.9, 8.9 Hz, 1H), 2.22 (dd, J=13.0, 6.6 Hz, 1H), 1.61 (t, J=19.5 Hz, 3H). LC/MS (ESI) m/z: 574 (M+H)⁺.

### Scheme 111: Synthesis of (S)—N—((R)-1-(4-carbamimidoylthiophen-2-yl)ethyl)-7-((4′-fluoro-[1,1′-biphenyl]-3-carbonyl)glycyl)-1,4-dioxa-7-azaspiro[4.4]nonane-8-carboxamide (Compound 112)

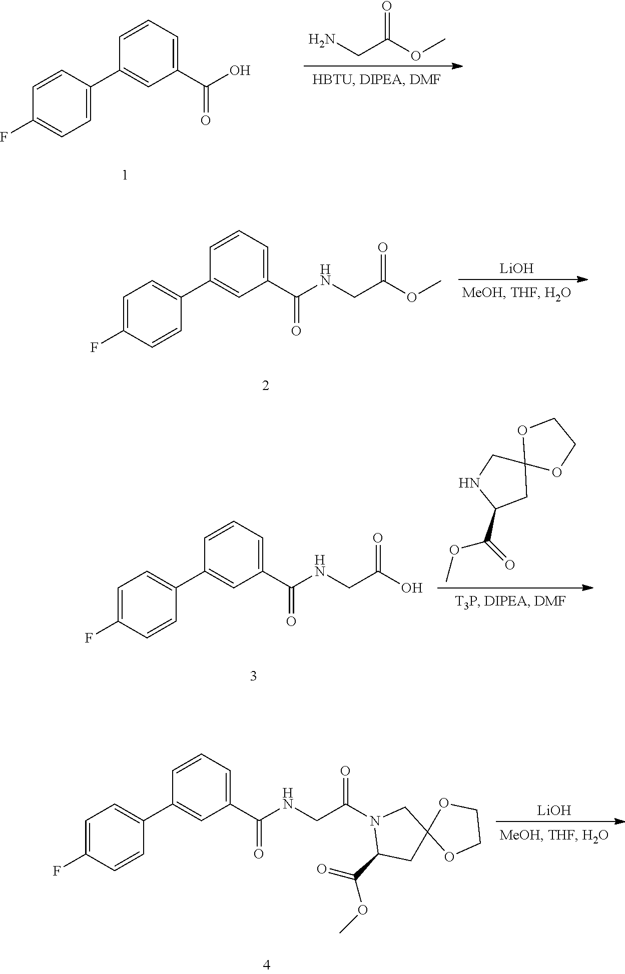

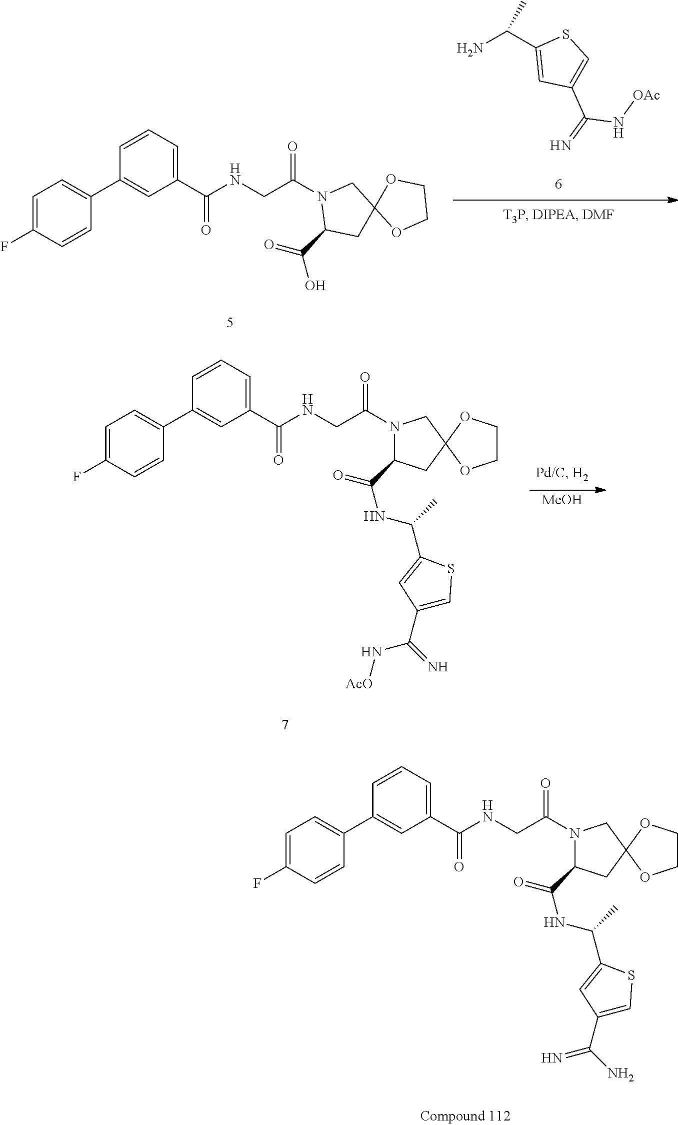

Compound 112 was prepared from 4′-fluoro-[1,1′-biphenyl]-3-carboxylic acid, methyl glycinate, methyl (S)-1,4-dioxa-7-azaspiro[4.4]nonane-8-carboxylate, and (R)—N-acetoxy-5-(1-aminoethyl) thiophene-3-carboximidamide based on the procedures set forth in Scheme 89. ¹H NMR (400 MHz, CD₃OD) δ 8.09 (d, J=6.3 Hz, 2H), 7.82 (dd, J=13.0, 7.9 Hz, 2H), 7.69 (dd, J=8.7, 5.2 Hz, 2H), 7.55 (t, J=7.8 Hz, 1H), 7.49 (s, 1H), 7.21 (t, J=8.8 Hz, 2H), 5.37-5.26 (m, 1H), 4.60-4.55 (m, 1H), 4.26-4.15 (m, 2H), 3.99 (dd, J=11.7, 7.2 Hz, 4H), 3.84-3.76 (m, 2H), 2.44 (dd, J=13.0, 8.9 Hz, 1H), 2.22 (dd, J=13.2, 6.3 Hz, 1H), 1.61 (dd, J=36.6, 6.9 Hz, 3H). LC/MS (ESI) m/z: 580 (M+H)⁺.

### Scheme 112: Synthesis of (S)-7-((9,9-difluoro-9H-fluorene-3-carbonyl)glycyl)-N—((R)-1-(4-(oxazol-2-yl)thiophen-2-yl)ethyl)-1,4-dioxa-7-azaspiro[4.4]nonane-8-carboxamide (Compound 113)

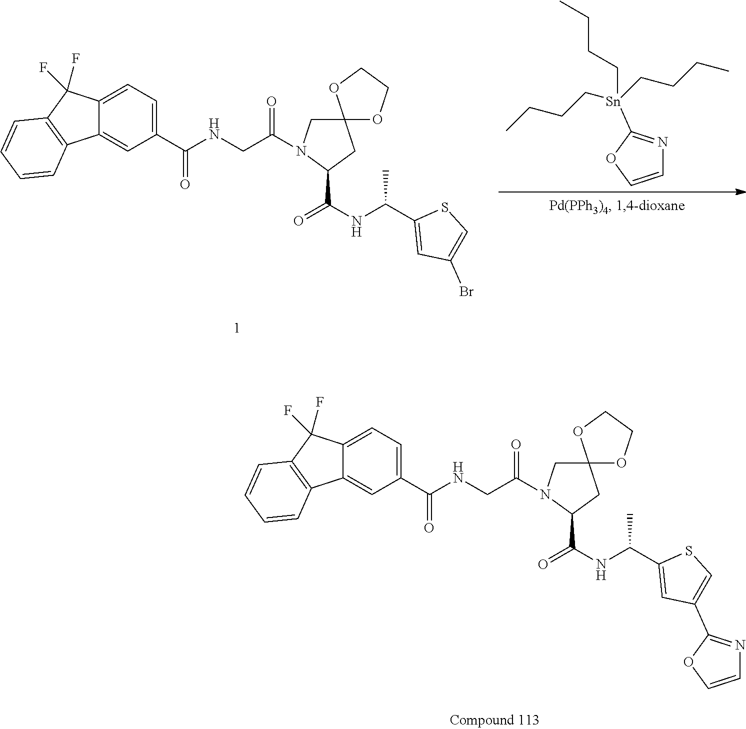 [SMILES: C[C@@H](NC(=O)[C@@H]1CC2(CN1C(=O)CNC(=O)c1ccc3c(c1)-c1ccccc1C3(F)F)OCCO2)c1cc(Br)cs1 ; CCC[CH2][Sn]([CH2]CCC)([CH2]CCC)[c]1ncco1 ; C[C@@H](NC(=O)[C@@H]1CC2(CN1C(=O)CNC(=O)c1ccc3c(c1)-c1ccccc1C3(F)F)OCCO2)c1cc(-c2ncco2)cs1]

To a solution of (S)—N—((R)-1-(4-bromothiophen-2-yl)ethyl)-7-((9,9-difluoro-9H-fluorene-3-carbonyl)glycyl)-1,4-dioxa-7-azaspiro[4.4]nonane-8-carboxamide (20 mg, 0.031 mmol) in 1,4-dioxane (1 mL) was added 2-(tributylstannyl)oxazole (22 mg, 0.062 mmol) and Pd(PPh₃)₄ (2 mg, 0.0017 mmol) at room temperature. The mixture was degassed under N₂ atmosphere for three times and stirred under a N₂ balloon at 90° C. overnight. The mixture was diluted with EtOAc, washed with water and brine, dried over anhydrous Na₂SO₄, filtered, and concentrated under reduced pressure to dryness. The residue was purified by prep-HPLC to give Compound 113 (2.6 mg, yield 13.2%) as a white solid. ¹H NMR (400 MHz, CD₃OD) δ 8.17 (d, J=8.3 Hz, 1H), 7.92 (s, 1H), 7.89-7.81 (m, 2H), 7.76-7.62 (m, 3H), 7.59-7.54 (m, 2H), 7.44 (t, J=7.4 Hz, 1H), 7.18 (s, 1H), 5.33 (d, J=7.2 Hz, 1H), 4.63-4.57 (m, 1H), 4.20 (s, 2H), 3.99 (d, J=13.2 Hz, 4H), 3.84-3.74 (m, 2H), 2.60-2.40 (m, 1H), 2.25 (dd, J=13.2, 5.3 Hz, 1H), 1.58 (d, J=6.8 Hz, 3H).LC/MS (ESI) m/z: 635 (M+H)⁺.

### Scheme 113: Synthesis of (S)-7-((9,9-difluoro-9H-fluorene-3-carbonyl)glycyl)-N—((R)-1-(4-(thiazol-2-yl)thiophen-2-yl)ethyl)-1,4-dioxa-7-azaspiro[4.4]nonane-8-carboxamide (Compound 114)

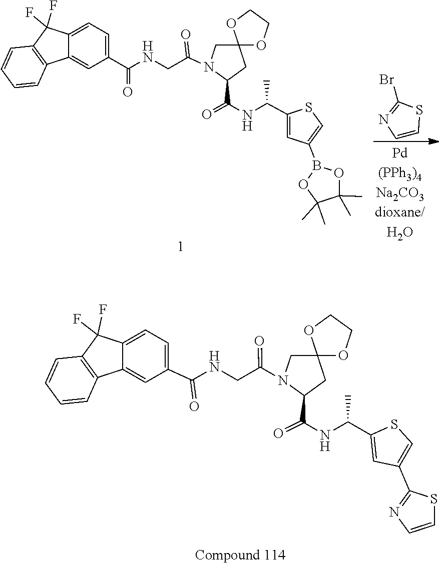 [SMILES: C[C@@H](NC(=O)[C@@H]1CC2(CN1C(=O)CNC(=O)c1ccc3c(c1)-c1ccccc1C3(F)F)OCCO2)c1cc(B2OC(C)(C)C(C)(C)O2)cs1 ; Brc1nccs1 ; C[C@@H](NC(=O)[C@@H]1CC2(CN1C(=O)CNC(=O)c1ccc3c(c1)-c1ccccc1C3(F)F)OCCO2)c1cc(-c2nccs2)cs1]

To a mixture of (S)-7-((9,9-difluoro-9H-fluorene-3-carbonyl)glycyl)-N—((R)-1-(4-(4,4,5,5-tetramethyl-1,3,2-dioxaborolan-2-yl)thiophen-2-yl)ethyl)-1,4-dioxa-7-azaspiro[4.4]nonane-8-carboxamide (25 mg, 0.036 mmol) in 1,4-dioxane/H₂O (1.6 mL/0.4 mL) was added 2-bromothiazole (12 mg, 0.072 mmol), Na₂CO₃ (11 mg, 0.1 mmol) and Pd(PPh₃)₄ (4 mg, 0.0035 mmol), and the mixture was degassed under N₂ atmosphere for three times and stirred under a N₂ balloon at 90° C. overnight. The mixture was filtered, and the filtrate was concentrated under reduced pressure to dryness. The residue was purified by prep-HPLC to give Compound 114 (5.4 mg, yield 23.1%) as a white solid. H NMR (400 MHz, CD₃OD) δ 8.16 (t, J=10.7 Hz, 1H), 7.83 (dd, J=25.0, 15.0 Hz, 2H), 7.77-7.60 (m, 4H), 7.60-7.47 (m, 2H), 7.44 (t, J=7.4 Hz, 1H), 7.29 (dd, J=36.7, 3.8 Hz, 1H), 5.39-5.27 (m, 1H), 4.59 (dt, J=17.5, 8.9 Hz, 1H), 4.26-4.15 (m, 2H), 3.99 (d, J=15.5 Hz, 4H), 3.83-3.74 (m, 2H), 2.56-2.39 (m, 1H), 2.28-2.16 (m, 1H), 1.56 (dd, J=18.0, 6.9 Hz, 3H). LC/MS (ESI) m/z: 651 (M+H)⁺.

### Scheme 114. (S)—N-((4-carbamimidoylthiophen-2-yl)methyl)-7-((2-methyl-[1,1′-biphenyl]-4-carbonyl)glycyl)-1,4-dioxa-7-azaspiro[4.4]nonane-8-carboxamide (Compound 115)

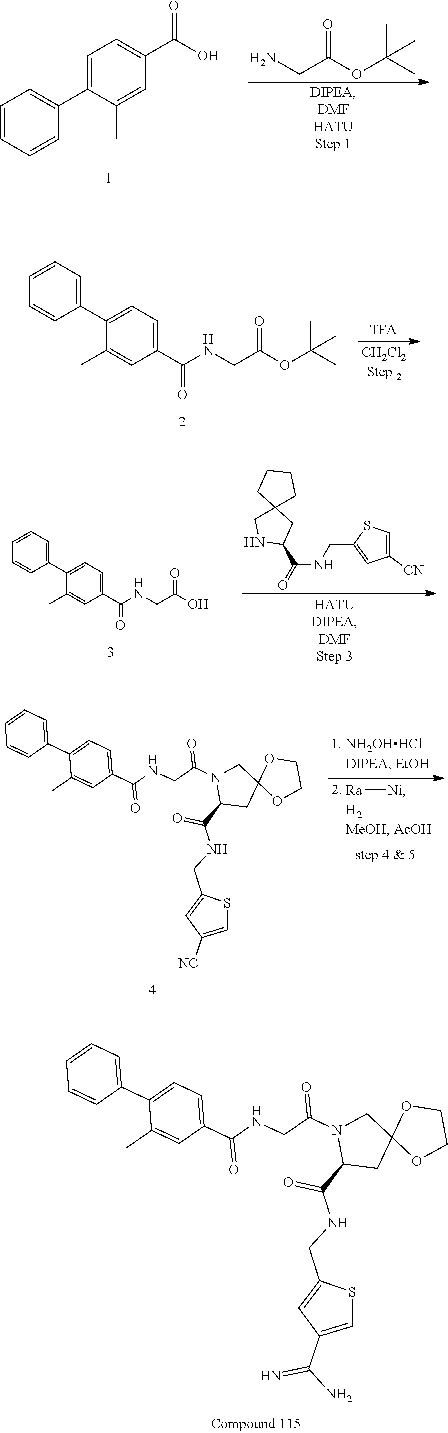

### Step 1: tert-butyl (2-methyl-[1,1′-biphenyl]-4-carbonyl)glycinate (2)

To a solution of 3-methyl-4-phenyl-benzoic acid, 1 (0.5 g, 2.36 mmol, 1 equiv.) in DMF (5 mL) was added tert-butyl 2-aminoacetate hydrochloride (0.5 g, 2.98 mmol, 1.2 equiv.), HATU (1.0 g, 2.63 mmol, 1.5 equiv.) and DIPEA (1.0 mL, 5.73 mmol, 3 equiv.). The reaction was stirred at room temperature for 1 h. Water (10 mL) was added stirred for 10 minutes. The product crashed out was collected by filtration and then dried to give 2 (0.66 g, 2.01 mmol, yield 85.43%) as a white solid. LC/MS (ESI) m/z: 326 (M+H)⁺.

### Step 2: (2-methyl-[1,1′-biphenyl]-4-carbonyl)glycine (3)

TFA (1.0 mL) was added to a solution of 2 (0.5 g, 1.54 mmol, 1.0 equiv.) in CH₂Cl₂ (4 mL) at ice-bath temperature. The reaction was stirred at room temperature for 3 hours and then concentrated to dryness to give 3 (0.35 g, 1.3 mmol, yield 84.58%) as a white solid which was used in the next step without further purification. LC/MS (ESI) m/z: 270 (M+H)⁺.

### Step 3: (S)—N-((4-cyanothiophen-2-yl)methyl)-7-((2-methyl-[1,1′-biphenyl]-4-carbonyl)glycyl)-1,4-dioxa-7-azaspiro[4.4]nonane-8-carboxamide (4)

To a solution of 3 (0.05 g, 0.19 mmol, 1.1 equiv.) in DMF (2 mL) was added (8S)—N-[(4-cyano-2-thienyl)methyl]-1,4-dioxa-7-azaspiro[4.4]nonane-8-carboxamide (0.04 g, 0.21 mmol, 1.0 equiv.), HATU (0.08 g, 0.21 mmol, 1.2 equiv.), and DIPEA (0.09 mL, 0.50 mmol, 3.0 equiv.). The reaction mixture was stirred at room temperature for 30 minutes. Water (2 mL) and EtOAc (5 mL) were added, and the two layers were separated. The aqueous layer was extracted with EtOAc 5 mL×2). The combined organic extracts were washed with brine, dried over Na₂SO₄ and concentrated. Purification by combi Flash, 4 g column, solvent A=CH₂Cl₂, solvent B=MecOH, 100% A to 5% B to give 4 (0.030 g, 0.055 mmol, 32.31% yield). LC/MS (ESI) m/z: 545 (M+H)⁺.

### Step 4 and 5: (S)—N-((4-carbamimidoylthiophen-2-yl)methyl)-7-((2-methyl-[1,1′-biphenyl]-4-carbonyl)glycyl)-1,4-dioxa-7-azaspiro[4.4]nonane-8-carboxamide (Compound 115)

To a solution of compound 4 (0.03 g, 0.06 mmol, 1 equiv.) in EtOH (1 mL) was added Hydroxylamine hydrochloride (10 mg, 0.14 mmol, 2.5 equiv.), and DIPEA (28.8 μL, 0.17 mmol, 3 equiv.). The reaction was stirred at room temperature for 24 hours. The solvent was removed under Rota vamp evaporation. The residue was taken in CH₂Cl₂, washed with brine, dried over Na₂SO₄, and concentrated to give a brown solid. To a solution of the solid obtained from the previous step in MeOH (1 mL) was added Ra—Ni (50 mass %) in H₂O (1.0 mg, 0.006 mmol, 0.1 equiv.), and AcOH (10 μL). The flask was evacuated and backfilled with H₂ in a balloon. The reaction was stirred at room temperature for 5 hours and then filtered through celite pad. The filtrate was concentrated under reduced pressure and then purified by HPLC to give Compound 115 (5 mg, 0.014 mmol, 16.16% yield). LC/MS (ESI) m/z: 562 (M+H)⁺. ¹H NMR (400 MHz, DMSO-d₆) δ 8.62 (d, J=14.4 Hz, 1H), 8.45-8.25 (m, 2H), 7.81 (s, 1H), 7.73 (d, J=7.6 Hz, 2H), 7.47 (t, J=7.4 Hz, 3H), 7.39 (dd, J=15.9, 7.3 Hz, 2H), 7.30 (d, J=7.9 Hz, 1H), 4.72 (s, 1H), 4.52 (d, J=5.5 Hz, 1H), 4.47-4.36 (m, 1H), 4.32 (t, J=7.8 Hz, 1H), 4.27-4.07 (m, 2H), 4.07-3.79 (m, 6H), 3.73-3.58 (m, 1H), 2.28 (s, 3H), 2.11-1.94 (m, 1H), 2.04 (dt, J=12.1, 6.0 Hz, 1H).

### Scheme 115: (S)—N-((4-carbamimidoylthiophen-2-yl)methyl)-7-((4′-(trifluoromethyl)-[1,1′-biphenyl]-4-carbonyl)glycyl)-1,4-dioxa-7-azaspiro[4.4]nonane-8-carboxamide (Compound 116)

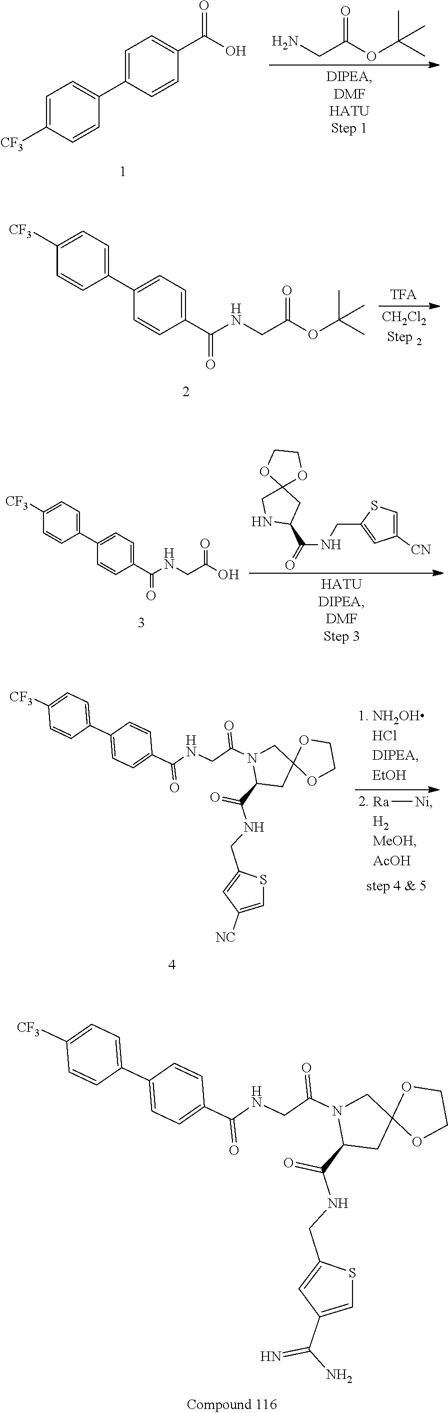

Compound 116 was prepared as a white solid from 4-[4-(trifluoromethyl)phenyl]benzoic acid, tert-butyl 2-aminoacetate. HCl, (8S)—N-[(4-cyano-2-thienyl)methyl]-1,4-dioxa-7-azaspiro[4.4]nonane-8-carboxamide, and hydroxylamine hydrochloride based on the procedures set forth in Scheme 114. LC/MS (ESI) m/z: 616 (M+H)⁺. ¹H NMR (400 MHz, DMSO-d₆) δ 9.09 (s, 1H) 9.05-8.81 (m, 1H), 8.70 (t, J=5.7 Hz, 1H), 8.60 (s, 1H), 8.35 (d, J=24.0 Hz, 3H), 7.95 (dd, J=12.3, 8.0 Hz, 3H), 7.75 (d, J=8.0 Hz, 2H), 7.64 (d, J=7.8 Hz, 2H), 7.50 (d, J=17.8 Hz, 2H), 7.30 (d, J=7.9 Hz, 2H), 4.69-4.34 (m, 3H), 4.17 (dd, J=16.6, 5.6 Hz, 1H), 4.04-3.81 (m, 2H), 3.79-3.13 (m, 2H), 2.78-2.65 (m, 1H), 2.04 (dt, J=12.1, 6.0 Hz, 1H).

### Scheme 116: Synthesis of (2S,4S*)—N—((R)-1-(4-carbamimidoylthiophen-2-yl)ethyl)-1-((9,9-difluoro-9H-fluorene-3-carbonyl)glycyl)-4-(methoxymethyl)pyrrolidine-2-carboxamide(Compound 117)

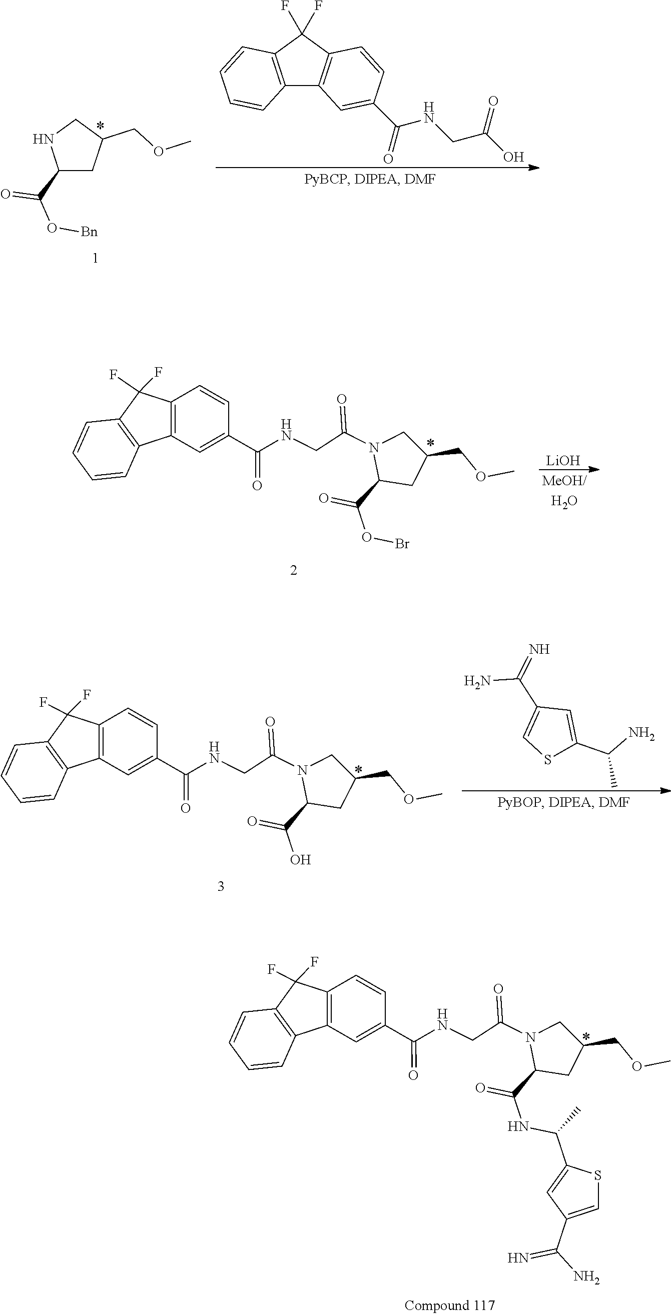

Compound 117 was prepared from benzyl (2S,4S*)-4-(methoxymethyl)pyrrolidine-2-carboxylate, (9,9-difluoro-9H-fluorene-3-carbonyl)glycine, and (R)-5-(1-aminoethyl)thiophene-3-carboximidamide based on the procedures set forth in Scheme 3. ¹H NMR (400 MHz, CD₃OD) δ 8.54 (s, 1H), 8.26-8.14 (m, 2H), 7.87 (t, J=8.6 Hz, 1H), 7.74 (dd, J=16.9, 7.6 Hz, 2H), 7.65 (d, J=7.4 Hz, 1H), 7.60-7.52 (m, 2H), 7.45 (t, J=7.4 Hz, 1H), 5.28 (q, J=7.0 Hz, 1H), 4.45 (t, J=8.4 Hz, 1H), 4.35 (d, J=16.6 Hz, 1H), 4.15 (d, J=16.6 Hz, 1H), 4.00-3.93 (m, 1H), 3.46 (dd, J=14.9, 8.3 Hz, 3H), 3.35 (s, 3H), 2.71-2.61 (m, 1H), 2.48-2.39 (m, 1H), 1.82-1.73 (m, 1H), 1.58 (d, J=7.0 Hz, 3H). LCMS (ESI) m/z=596 (M+H)⁺.

### Scheme 117: Synthesis of (2S,4R*)—N—((R)-1-(4-carbamimidoylthiophen-2-yl)ethyl)-1-((9,9-difluoro-9H-fluorene-3-carbonyl)glycyl)-4-(methoxymethyl)pyrrolidine-2-carboxamide (Compound 118)

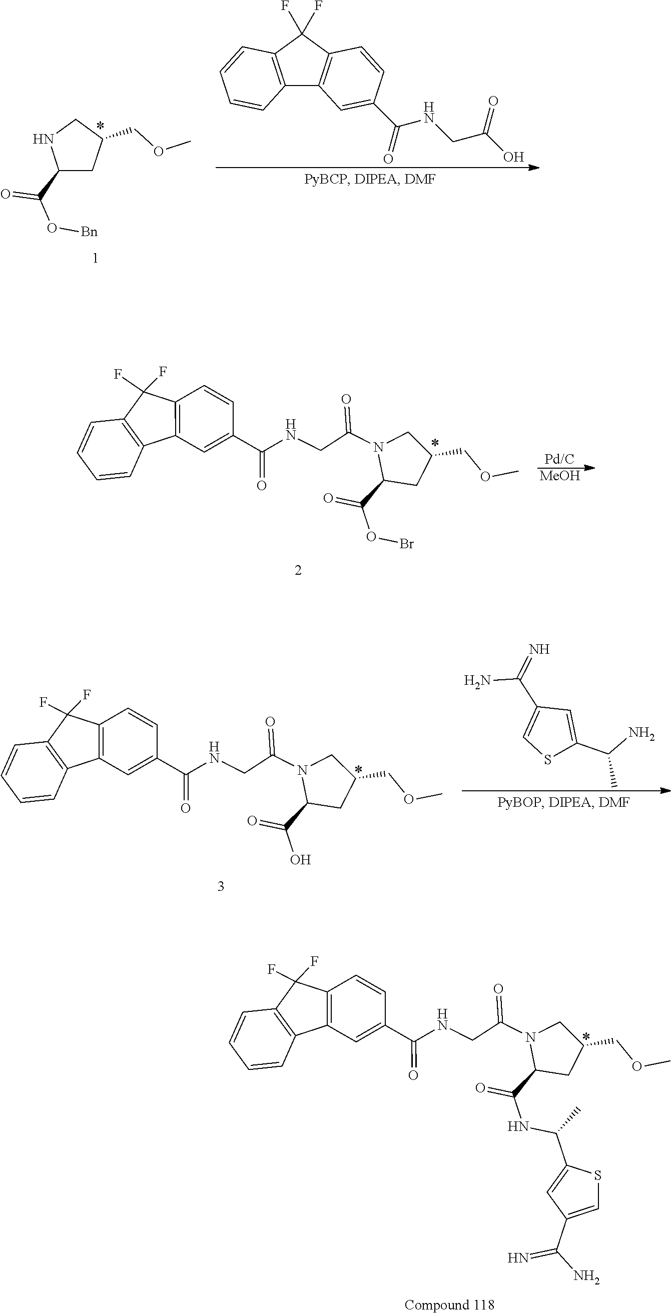

Compound 118 was prepared as a white solid from benzyl (2S,4R*)-4-(methoxymethyl)pyrrolidine-2-carboxylate, (9,9-difluoro-9H-fluorene-3-carbonyl)glycine, and (R)-5-(1-aminoethyl)thiophene-3-carboximidamide based on the procedures set forth in Scheme 12. ¹H NMR (400 MHz, CD₃OD) δ 8.56 (s, 1H), 8.25-8.16 (m, 2H), 7.87 (t, J=8.3 Hz, 1H), 7.74 (dd, J=18.4, 7.8 Hz, 2H), 7.65 (d, J=7.3 Hz, 1H), 7.58 (t, J=7.6 Hz, 1H), 7.46 (dd, J=14.5, 7.0 Hz, 2H), 5.35-5.25 (m, 1H), 4.54 (s, 1H), 4.30-4.12 (m, 2H), 3.95-3.88 (m, 1H), 3.52-3.40 (m, 3H), 3.36 (d, J=8.3 Hz, 3H), 2.72 (dd, J=37.1, 30.1 Hz, 1H), 2.08 (dd, J=21.3, 12.3 Hz, 2H), 1.61 (t, J=16.4 Hz, 3H). LC/MS (ESI) (m/z): 596 (M+H)⁺.

### Scheme 118: Synthesis of (2S,4R)—N—((R)-1-(4-carbamimidoylthiophen-2-yl)ethyl)-1-((9,9-difluoro-9H-fluorene-3-carbonyl)glycyl)-4-(o-tolyl)pyrrolidine-2-carboxamide (Compound 119)

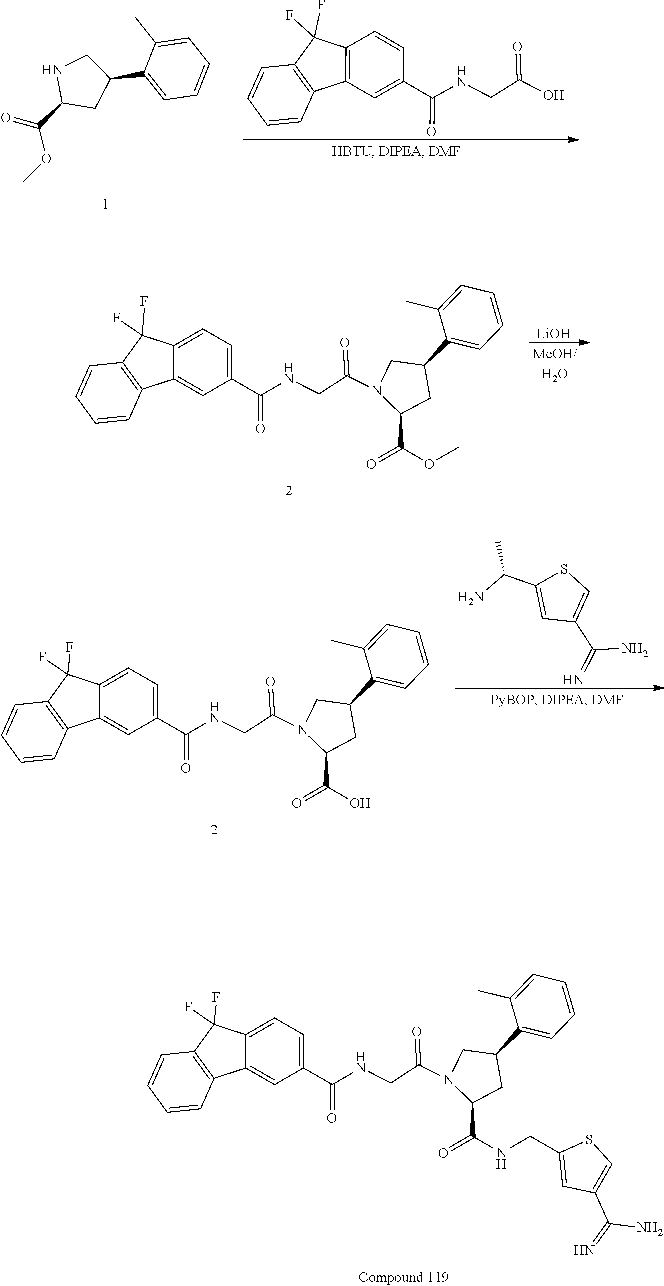

Compound 119 was prepared as a white solid from methyl (2S,4R)-4-(o-tolyl)pyrrolidine-2-carboxylate, (9,9-difluoro-9H-fluorene-3-carbonyl)glycine, and (R)-5-(1-aminoethyl)thiophene-3-carboximidamide based on the procedures set forth in Scheme 3. ¹H NMR (400 MHz, CD₃OD) δ 8.53 (s, 1H), 8.23 (t, J=13.7 Hz, 2H), 7.89 (d, J=7.5 Hz, 1H), 7.74 (dd, J=15.3, 7.6 Hz, 2H), 7.66 (d, J=7.3 Hz, 1H), 7.60-7.55 (m, 2H), 7.45 (t, J=7.6 Hz, 1H), 7.36 (d, J=7.3 Hz, 1H), 7.22-7.15 (m, 3H), 5.32 (d, J=6.2 Hz, 1H), 4.58-4.39 (m, 3H), 4.29-4.13 (m, 2H), 3.71 (dd, J=35.6, 25.3 Hz, 2H), 2.42 (d, J=16.7 Hz, 3H), 2.19-1.94 (m, 1H), 1.62 (t, J=12.8 Hz, 3H). LCMS (ESI) (m/z): 642 (M+H)⁺.

### Scheme 119: Synthesis of (S)—N—((R)-1-(4-carbamimidoylthiophen-2-yl)ethyl)-7-((4′-methyl-[1,1′-biphenyl]-3-carbonyl)glycyl)-1,4-dioxa-7-azaspiro[4.4]nonane-8-carboxamide (Compound 120)

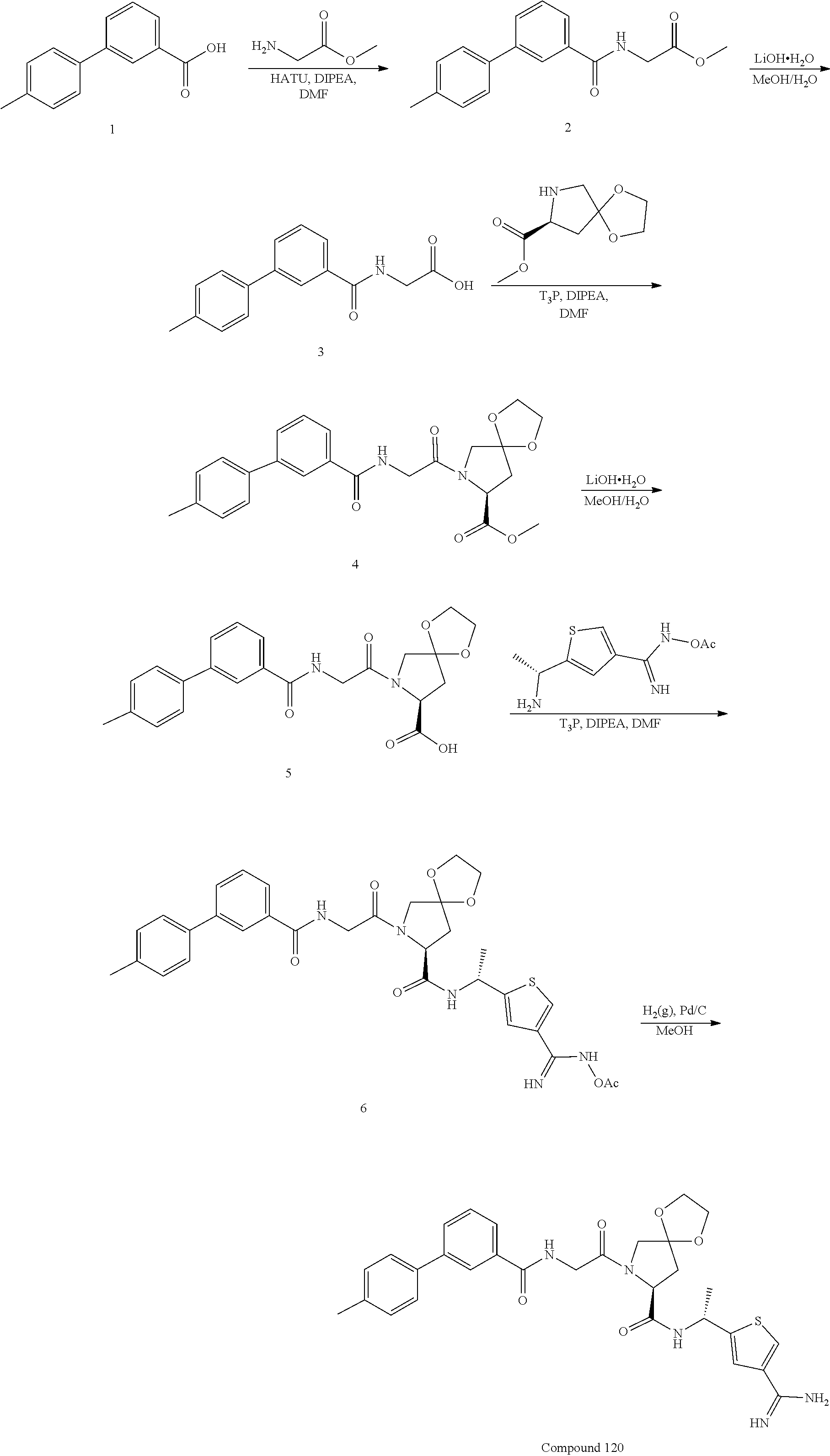

Compound 120 was prepared as a white solid from 4′-methyl-[1,1′-biphenyl]-3-carboxylic acid, methyl glycinate, methyl (S)-1,4-dioxa-7-azaspiro[4.4]nonane-8-carboxylate, and (R)—N-acetoxy-5-(1-aminoethyl)thiophene-3-carboximidamide based on the procedures set forth in Scheme 89. ¹H NMR (400 MHz, CD₃OD) δ 8.50 (s, 1H), 8.22 (t, J=10.3 Hz, 1H), 8.10 (s, 1H), 7.80 (dd, J=7.8, 1.6 Hz, 2H), 7.54 (dd, J=13.7, 7.3 Hz, 4H), 7.28 (d, J=7.9 Hz, 2H), 5.28 (q, J=7.0 Hz, 1H), 4.65-4.54 (m, 1H), 4.21 (d, J=17.1 Hz, 2H), 4.00 (s, 2H), 4.00-3.95 (m, 2H), 3.85-3.77 (m, 2H), 2.44 (dd, J=13.2, 9.1 Hz, 1H), 2.39 (s, 3H), 2.21 (dd, J=13.1, 6.5 Hz, 1H), 1.60 (t, J=18.8 Hz, 3H). LC/MS (ESI) m/z: 576 (M+H)⁺.

### Scheme 120: Synthesis of (1S,3S,5S)—N-[(1R)-1-(4-carbamimidoylthiophen-2-yl)ethyl]-2-{2-[(9,9-difluorofluoren-2-yl)formamido]acetyl}-5-methyl-2-azabicyclo[3.1.0]hexane-3-carboxamide (Compound 121)

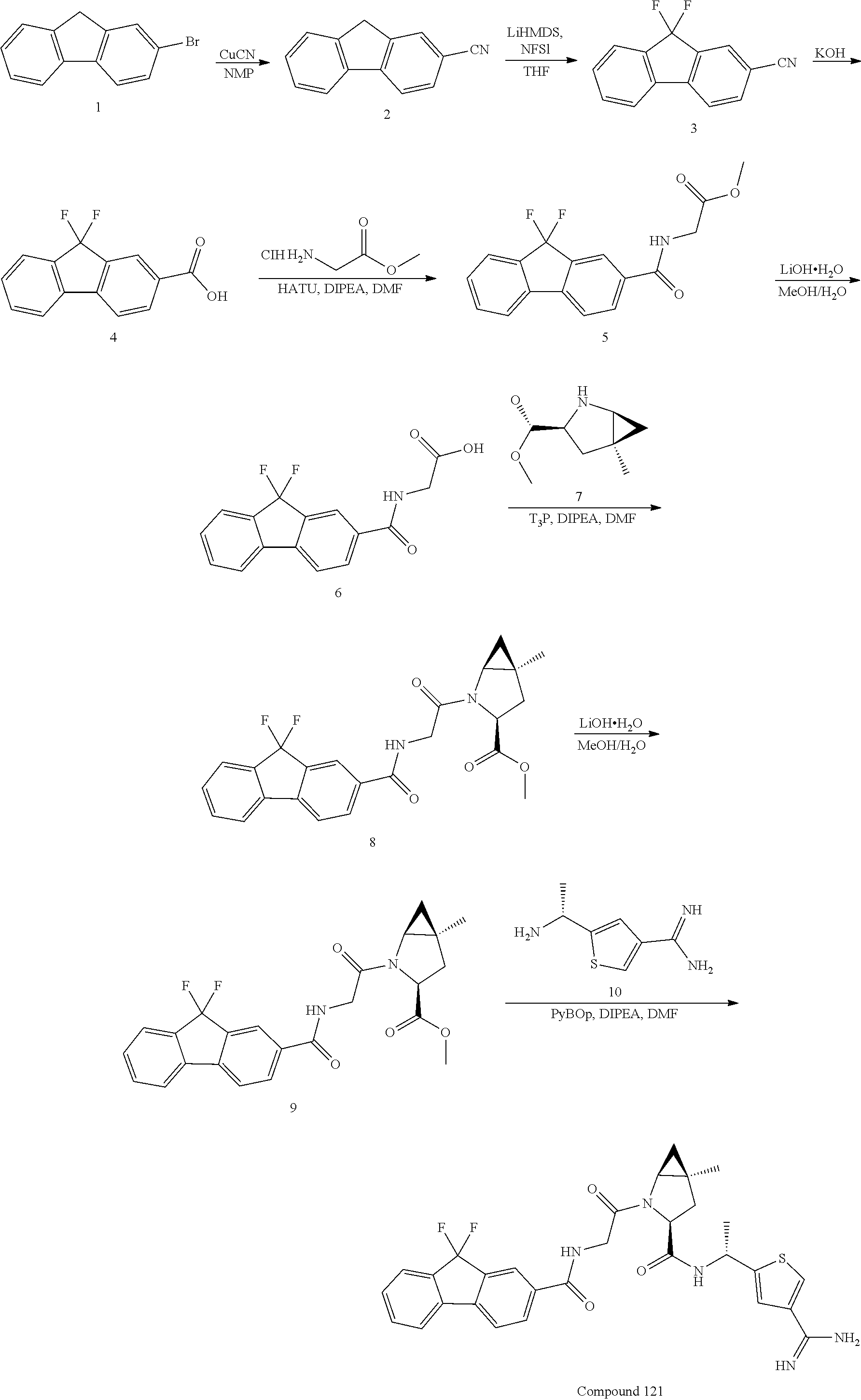

### Step 1: 2-cyano-9H-fluorene (2)

To a solution of 2-bromo-9H-fluorene (2.45 g, 10 mmol) in NMP (25 mL) was added CuCN (2.72 g, 30 mmol) under N₂ atmosphere and the reaction mixture was stirred at 160° C. for 6 hours. The mixture was diluted with EtOAc, washed with brine, dried over anhydrous Na₂SO₄, filtered, and concentrated under reduced pressure to dryness. The residue was purified by flash chromatography on silica gel (PE:EtOAc=3:1) to afford 2-cyano-9H-fluorene (1.8 g, yield 94.2%) as a yellow solid. LC/MS (ESI) (m/z): 192 (M+H)⁺.

### Step 2: 9,9-difluorofluorene-2-carbonitrile (3)

To a solution of 2-cyano-9H-fluorene (1 g, 5.23 mmol) in THF (20 mL) was added LiHMDS (21 mL, 20.92 mmol) at −78° C. and stirred for 0.5 hours before NFSI (4.05 g, 15.69 mmol) was added at −78° C. The mixture was stirred at −78° C. under N₂ atmosphere for additional 4 hours before diluted with water and extracted with EtOAc (3×100 mL). The combined organic layers were washed with brine, dried over anhydrous Na₂SO₄, filtered, and concentrated to dryness under reduced pressure. The residue was purified by flash chromatography on silica gel (PE:EtOAc=10:1) to give 9,9-difluorofluorene-2-carbonitrile (850 mg, yield 71.5%) as a yellow solid. LC/MS (ESI) (m/z): 228 (M+H)⁺.

### Step 3: 9,9-difluorofluorene-2-carboxylic acid (4)

To a mixture of 9,9-difluorofluorene-2-carbonitrile (800 mg, 3.52 mmol) in MeOH (10 mL) and H₂O (10 mL) was added KOH (592 mg, 10.56 mmol). The mixture was stirred at 60° C. overnight. The reaction mixture was acidified with 1 N aq·HCl solution to pH˜4 and extracted with EtOAc twice. The combined organic layers were washed with brine, dried over anhydrous Na₂SO₄, filtered, and concentrated to dryness under reduced pressure to give 9,9-difluorofluorene-2-carboxylic acid (500 mg, yield 57.7%). LC/MS (ESI) m/z: 247 (M+H)⁺.

### Step 4: methyl 2-[(9,9-difluorofluoren-2-yl)formamido]acetate (5)

To a solution of 9,9-difluorofluorene-2-carboxylic acid (300 mg, 1.22 mmol) in DMF (5 mL) was added methyl 2-aminoacetate hydrochloride (184 mg, 1.46 mmol), DIPEA (472 mg, 3.66 mmol) and HATU (556 mg, 1.46 mmol) under N₂ atmosphere and the reaction mixture was stirred at room temperature for 1 hour. The mixture was diluted with water and extracted with EtOAc. the organic layer was washed with brine, dried over anhydrous Na₂SO₄, filtered, and concentrated under reduced pressure to dryness. The residue was purified by flash chromatography on silica gel (PE:EtOAc=3:1) to afford methyl 2-[(9,9-difluorofluoren-2-yl)formamido]acetate (130 mg, yield 33.6%) as a white solid. LC/MS (ESI) m/z: 318 (M+H)⁺.

### Step 5: [(9,9-difluorofluoren-2-yl)formamido]acetic acid (6)

To a mixture of methyl 2-[(9,9-difluorofluoren-2-yl)formamido]acetate (130 mg, 0.41 mmol) in MeOH (1.6 mL) and H₂O (0.4 mL) was added LiOH (52 mg, 1.23 mmol) and the reaction mixture was stirred at room temperature for 4 hours. The mixture was acidified with 1 N aq·HCl to pH-4 and extracted with EtOAc twice. The combined organic layers were washed with brine, dried over anhydrous Na₂SO₄, filtered, and concentrated to dryness under reduced pressure to afford [(9,9-difluorofluoren-2-yl)formamido]acetic acid (110 mg, yield 88.5%)as a white solid. LC/MS (ESI) m/z: 304 (M+H)⁺.

### Step 6: ethyl (1S,3S,5S)-5-methyl-2-azabicyclo[3.1.0]hexane-3-carboxylate hydrochloride (7)

To a solution of 2-tert-butyl 3-ethyl (1S,3S,5S)-5-methyl-2-azabicyclo[3.1.0]hexane-2,3-dicarboxylate (60 mg, 0.22 mmol) in DCM (2 mL) was added HCl/1,4-dioxane (0.5 mL, 2 mmol) at room temperature and the mixture was stirred for 2 hours. The reaction mixture was concentrated to give ethyl (1S,3S,5S)-5-methyl-2-azabicyclo[3.1.0]hexane-3-carboxylate hydrochloride (45 mg, yield 98.2%) as a brown solid which was used directly in next step without further purification.

### Step 7: ethyl (1S,3S,5S)-2-(2-[(9,9-difluorofluoren-2-yl)formamido]acetyl-5-methyl-2-azabicyclo[3.1.0]hexane-3-carboxylate (6)

To mixture of [(9,9-difluorofluoren-2-yl)formamido]acetic acid (40 mg, 0.13 mmol), ethyl (1S,3S,5S)-5-methyl-2-azabicyclo[3.1.0]hexane-3-carboxylate hydrochloride (45 mg, 0.22 mmol) and DIPEA (51 mg, 0.40 mmol) in DMF (0.5 mL) was added T₃P (126 mg, 0.2 mmol, 50% in EtOAc) under N₂ atmosphere and the reaction mixture was stirred at room temperature for 1 hour. The mixture was diluted with EtOAc and washed with saturated aq·NaHCO₃ solution. The aqueous layer was extracted with EtOAc twice and the combined organic layers were washed with brine, dried over anhydrous Na₂SO₄, filtered, and concentrated under reduced pressure to dryness. The residue was purified by flash chromatography on silica gel (PE:EtOAc=3:1) to afford ethyl (1S,3S,5S)-2-{2-[(9,9-difluorofluoren-2-yl)formamido]acetyl}-5-methyl-2-azabicyclo[3.1.0]hexane-3-carboxylate (48 mg, yield 80.1%) as a yellow oil. LC/MS (ESI) m/z: 455 (M+H)⁺.

### Step 8: (1S,3S,5S)-2-(2-[(9,9-difluorofluoren-2-yl)formamido]acetyl)-5-methyl-2-azabicyclo[3.1.0]hexane-3-carboxylic acid

To a solution of methyl (1S,3S,5S)-2-{2-[(9,9-difluorofluoren-2-yl)formamido]acetyl}-5-methyl-2-azabicyclo[3.1.0]hexane-3-carboxylate (48 mg, 0.11 mmol) in MeOH (0.8 mL) and H₂O (0.2 mL) was added LiOH (14 mg, 0.33 mmol) under N₂ atmosphere and the reaction mixture was stirred at room temperature for 3 hours. The mixture was acidified with 1 N aq·HCl to pH-3, extracted with EtOAc twice. The combined organic layers were washed with brine, dried over anhydrous Na₂SO₄, filtered, and concentrated under reduced pressure to give (1 S,3S,5S)-2-{2-[(9,9-difluorofluoren-2-yl)formamido]acetyl}-5-methyl-2-azabicyclo[3.1.0]hexane-3-carboxylic acid (40 mg, yield 86.1%) as a white solid. LC/MS (ESI) m/z:427 (M+H)⁺.

### Step 9: (1S,3S,5S)—N-[(1R)-1-(4-carbamimidoylthiophen-2-yl)ethyl]-2-{2-[(9,9-difluorofluoren-2-yl)formamido]acetyl}-5-methyl-2-azabicyclo[3.1.0]hexane-3-carboxamide (Compound 121)

To a mixture of (1 S,3S,5S)-2-{2-[(9,9-difluorofluoren-2-yl)formamido]acetyl}-5-methyl-2-azabicyclo[3.1.0]hexane-3-carboxylic acid (30 mg, 0.07 mmol), 5-[(1R)-1-aminoethyl]thiophene-3-carboximidamide (24 mg, 0.14 mmol) and DIPEA (27 mg, 0.21 mmol) in DMF (0.5 mL) was added PyBop (37 mg, 0.07 mmol) under N₂ atmosphere and the reaction mixture was stirred at room temperature for 1 hour. The mixture was diluted with EtOAc and washed with saturated aq·NH₄Cl solution. The aqueous layer was extracted with EtOAc twice and the combined organic layers were washed with brine, dried over anhydrous Na₂SO₄, filtered, and concentrated under reduced pressure to dryness. The residue was purified by TLC and prep-HPLC to afford Compound 121 (6.5 mg, yield 16.1%) as a white solid. ¹H NMR (400 MHz, CD₃OD) δ 8.54 (s, 1H), 8.23 (d, J=12.5 Hz, 1H), 8.11 (s, 1H), 8.06 (d, J=7.7 Hz, 1H), 7.85-7.78 (m, 2H), 7.67 (d, J=7.8 Hz, 1H), 7.59 (t, J=7.4 Hz, 1H), 7.48 (dd, J=14.9, 7.2 Hz, 2H), 5.27-5.19 (m, 1H), 4.59 (s, 2H), 4.45 (d, J=16.5 Hz, 1H), 4.29 (d, J=16.5 Hz, 1H), 2.20-2.15 (m, 1H), 1.59 (d, J=7.0 Hz, 3H), 1.31 (s, 3H), 1.22 (d, J=3.5 Hz, 1H), 0.90 (s, 1H), 0.79 (t, J=5.5 Hz, 1H). LC/MS (ESI) m/z: 578 (M+H)⁺.

### Scheme 121: (S)—N-((4-(2-aminopyridin-3-yl)thiophen-2-yl)methyl)-7-((4-(4-fluorophenoxy)benzoyl)glycyl)-1,4-dioxa-7-azaspiro[4.4]nonane-8-carboxamide (Compound 122)

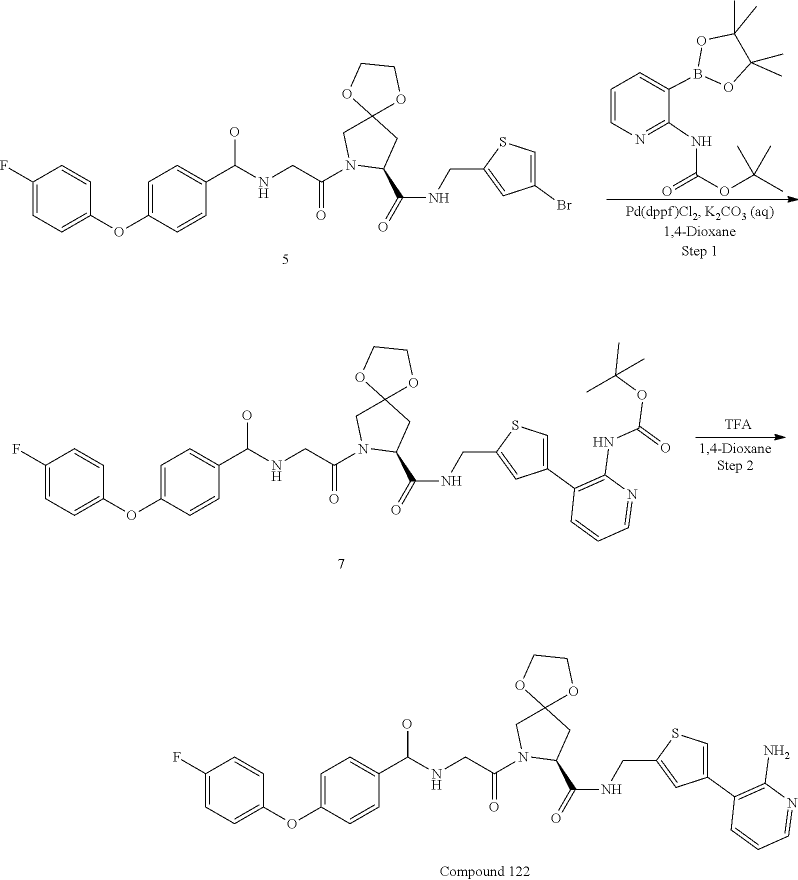

Compound 122 was prepared as a beige solid from (S)—N-((4-bromothiophen-2-yl)methyl)-7-((4-(4-fluorophenoxy)benzoyl)glycyl)-1,4-dioxa-7-azaspiro[4.4]nonane-8-carboxamide and tert-butyl (3-(4,4,5,5-tetramethyl-1,3,2-dioxaborolan-2-yl)pyridin-2-yl)carbamate based on the procedures set forth in Scheme 102. LC/MS (ESI) m/z: 632 (M+H)⁺. ¹H NMR (400 MHz, DMSO) δ 1.98-2.39 (m, 2H), 3.52-4.05 (m, 6H), 5.50-5.67 (s, 2H), 7.12-7.20 (s, 3H), 7.23-7.31 (s, 2H), 4.06-4.79 (m, 5H), 6.91-7.07 (d, J=8.3 Hz, 2H), 6.48-6.65 (s, 1H), 7.34-7.54 (m, 1H), 7.84-7.90 (s, 4H), 8.12-8.22 (s, 1H), 8.46-8.65 (m, 2H).

### Scheme 122: (S)—N-((4-(6-aminopyridin-3-yl)thiophen-2-yl)methyl)-7-((4-(4-fluorophenoxy)benzoyl)glycyl)-1,4-dioxa-7-azaspiro[4.4]nonane-8-carboxamide (Compound 123)

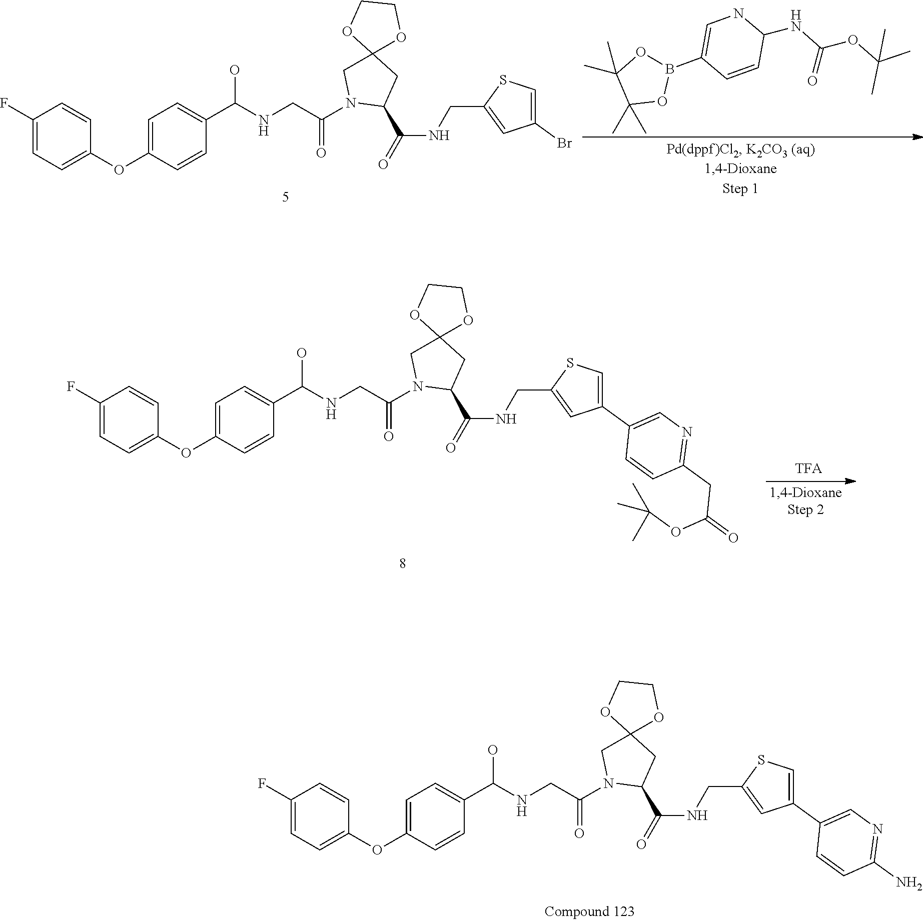

Compound 123 was prepared as a beige solid from (S)—N-((4-bromothiophen-2-yl)methyl)-7-((4-(4-fluorophenoxy)benzoyl)glycyl)-1,4-dioxa-7-azaspiro[4.4]nonane-8-carboxamide and tert-butyl (5-(4,4,5,5-tetramethyl-1,3,2-dioxaborolan-2-yl)pyridin-2-yl)carbamate based on the procedures set forth in Scheme 102. LC/MS (ESI) m/z: 632 (M+H)⁺. ¹H NMR (400 MHz, DMSO) δ 2.03-2.13 (m, 1H), 2.30-2.40 (dd, J=13.3, 8.9 Hz, 1H), 3.63-3.75 (t, J=11.0 Hz, 1H), 3.78-3.85 (d, J=10.8 Hz, 1H), 3.89-4.05 (tq, J=11.1, 5.8 Hz, 5H), 4.09-4.23 (dd, J=16.6, 5.7 Hz, 1H), 4.39-4.48 (d, J=6.3 Hz, 2H), 5.94-6.01 (d, J=8.6 Hz, 2H), 6.42-6.53 (t, J=8.1 Hz, 1H), 6.99-7.08 (d, J=8.3 Hz, 2H), 7.13-7.22 (dt, J=8.9, 4.5 Hz, 2H), 7.24-7.37 (m, 3H), 7.43-7.51 (d, J=13.3 Hz, 1H), 7.62-7.71 (dt, J=8.3, 3.7 Hz, 1H), 7.87-7.94 (dd, J=8.9, 3.2 Hz, 2H), 8.18-8.28 (m, 1H), 8.47-8.54 (t, J=6.0 Hz, 1H), 8.56-8.67 (t, J=5.6 Hz, 1H).

### Scheme 123: (S)-7-((4-(4-fluorophenoxy)benzoyl)glycyl)-N-(thiophen-2-ylmethyl)-1,4-dioxa-7-azaspiro [4.4]nonane-8-carboxamide (Compound 124)

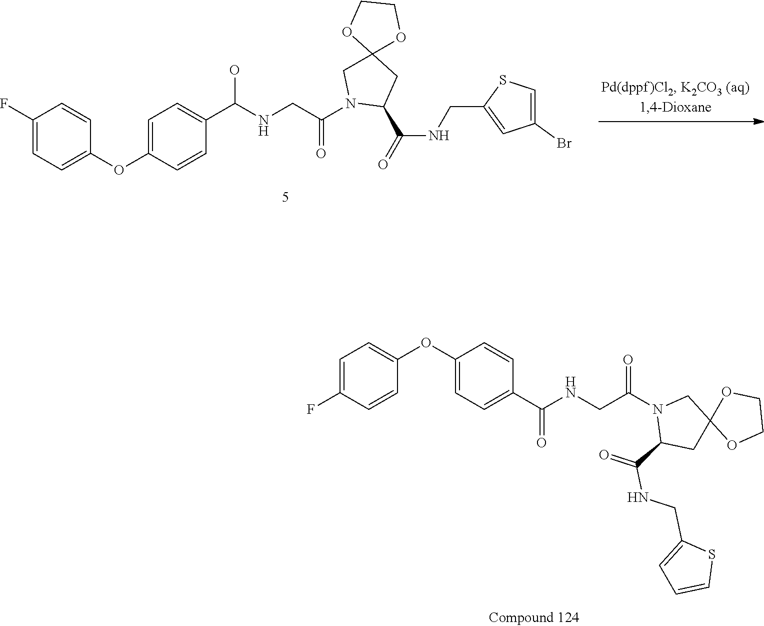

To a solution of (S)—N-((4-bromothiophen-2-yl)methyl)-7-((4-(4-fluorophenoxy)benzoyl)glycyl)-1,4-dioxa-7-azaspiro[4.4]nonane-8-carboxamide, 5 (0.0500 g, 0.0808 mmol, 1.0 equiv.) in 1,4-Dioxane (2.00 mL) was added Potassium phosphate in H₂O (2.00 mL, 4.00 mmol, 2.00 mol/L, 49.5 equiv.). Ar gas was passed through the solution for 2 min followed by the addition of Pd(dppf)Cl₂·CH₂Cl₂ (0.0100 g, 0.0130 mmol, 0.16 equiv.). The tube was sealed and placed in a microwave synthesizer for 30 mins at 110° C. LC/MS indicated the completion of the reaction. The mixture was then passed through a celite plug and the filtrate was concentrated. The residue was purified by ISCO column chromatographic purification system (12 g gold column) using 0-5% MeOH in DCM to obtain desired product Compound 124 (0.0100 g, 0.0185 mmol, 22.9% yield) as a brown solid. LC/MS (ESI) m/z: 540 (M+H)⁺. ¹H NMR (400 MHz, DMSO) δ 1.97-2.36 (m, 2H), 3.57-3.81 (m, 2H), 3.82-4.02 (dtd, J=14.8, 7.1, 3.4 Hz, 5H), 4.04-4.21 (m, 1H), 4.31-4.56 (m, 3H), 6.89-7.05 (m, 5H), 7.11-7.19 (dd, J=9.0, 4.4 Hz, 2H), 7.22-7.32 (t, J=8.8 Hz, 2H), 7.33-7.42 (dd, J=13.2, 5.1 Hz, 1H), 7.85-7.92 (dd, J=8.8, 2.7 Hz, 2H), 8.42-8.49 (t, J=6.0 Hz, 1H), 8.54-8.61 (t, J=5.6 Hz, 1H).

### Scheme 124: Synthesis of (S)—N—((R)-1-(4-(N-acetoxycarbamimidoyl)thiophen-2-yl) ethyl)-7-((9,9-difluoro-9H-fluorene-3-carbonyl)glycyl)-1,4-dioxa-7-azaspiro[4.4]nonane-8-carboxamide (Compound 125)

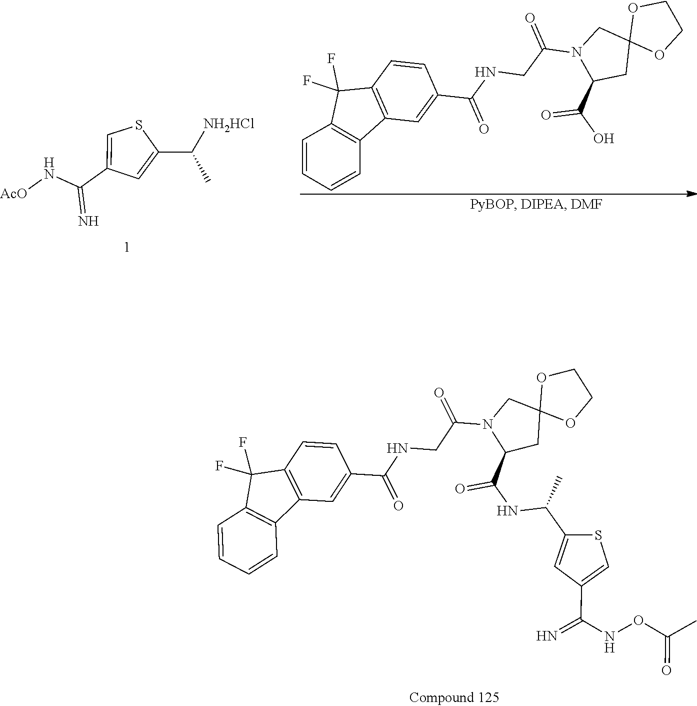

Compound 125 was prepared as a white solid from {5-[(1R)-1-aminoethyl]thiophen-3-yl}methanimidamido acetate and (8S)-7-{2-[(9,9-difluorofluoren-3-yl)formamido]acetyl}-1,4-dioxa-7-azaspiro[4.4]nonane-8-carboxylic acid based on the procedures set forth in Scheme 3. ¹H NMR (400 MHz, CD₃OD) δ 8.16 (s, 1H), 7.88-7.84 (m, 1H), 7.81-7.77 (m, 1H), 7.77-7.72 (m, 1H), 7.69 (d, J=8.0 Hz, 1H), 7.64 (d, J=7.6 Hz, 1H), 7.56 (t, J=7.6 Hz, 1H), 7.48-7.41 (m, 2H), 5.38-5.24 (m, 1H), 4.75-4.56 (m, 1H), 4.30-4.10 (m, 2H), 4.05-3.96 (m, 4H), 3.84-3.73 (m, 2H), 2.63-2.40 (m, 1H), 2.37-2.20 (m, 1H), 2.12 (d, J=11.2 Hz, 3H), 1.59 (dd, J=36.0, 36.0 Hz, 3H). LC/MS (ESI) (m/z): 668 (M+H)⁺.

### Scheme 125: Synthesis of (S)—N—((R)-1-(4-carbamimidoylthiophen-2-yl)ethyl)-7-((4′-methyl-[1,1′-biphenyl]-4-carbonyl)glycyl)-1,4-dioxa-7-azaspiro[4.4]nonane-8-carboxamide (Compound 126)

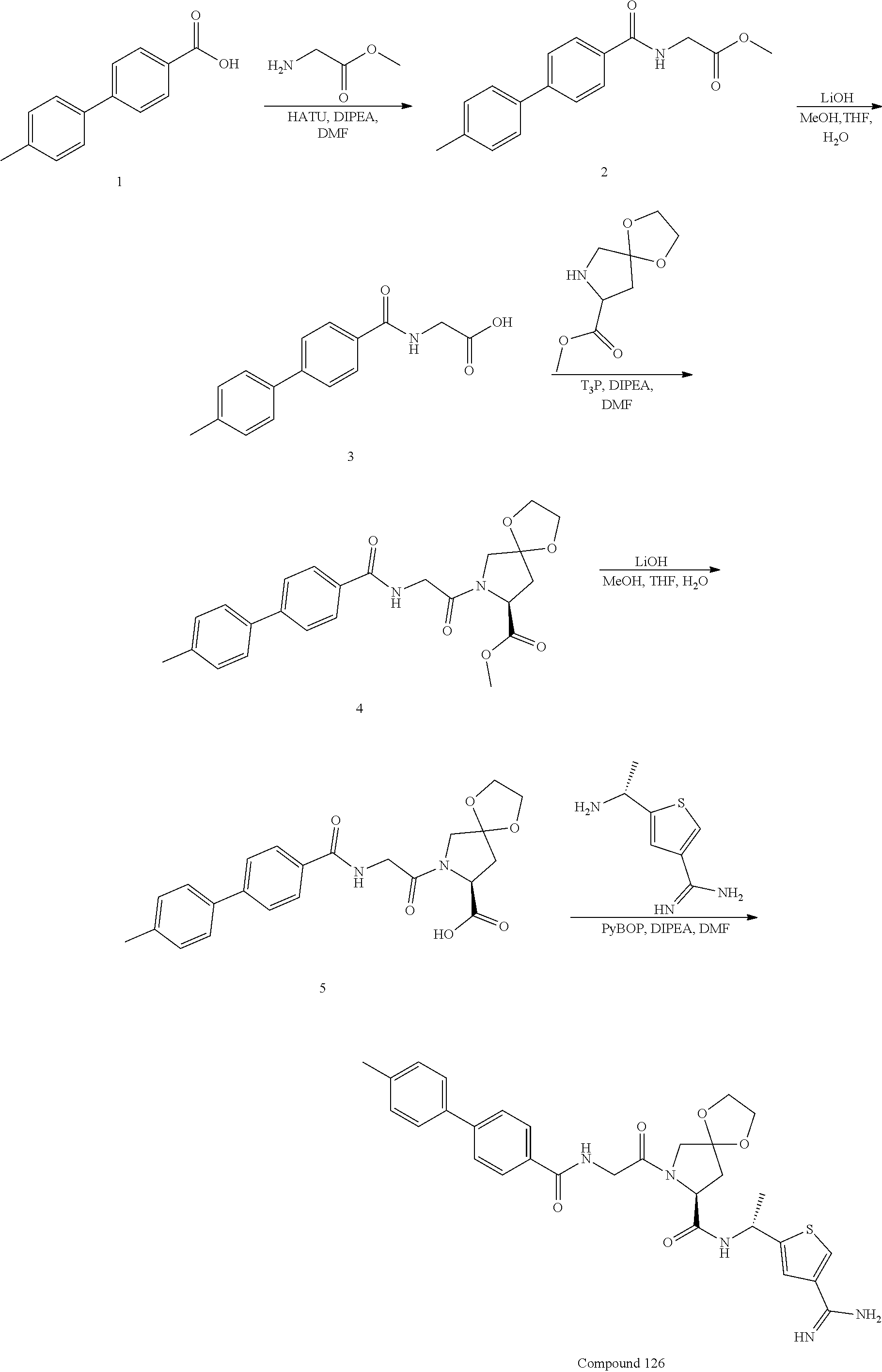

Compound 126 was prepared as a white solid from 4′-methyl-[1,1′-biphenyl]-4-carboxylic acid, methyl glycinate, methyl (S)-1,4-dioxa-7-azaspiro[4.4]nonane-8-carboxylate, and (R)-5-(1-aminoethyl)thiophene-3-carboximidamide bvased on the procedures set forth in Scheme 7. ¹H NMR (400 MHz, CD₃OD) δ 8.56 (s, 1H), 8.24 (t, J=5.8 Hz, 1H), 7.92 (d, J=8.3 Hz, 2H), 7.71 (d, J=8.3 Hz, 2H), 7.58-7.53 (m, 3H), 7.29 (d, J=7.9 Hz, 2H), 5.32 (dq, J=13.8, 6.8 Hz, 1H), 4.59 (d, J=6.7 Hz, 1H), 4.17 (d, J=13.9 Hz, 2H), 4.02-3.94 (m, 4H), 3.80 (q, J=10.9 Hz, 2H), 2.44 (dd, J=13.1, 8.9 Hz, 1H), 2.39 (s, 3H), 2.22 (dd, J=13.1, 6.5 Hz, 1H), 1.63 (dd, J=36.7, 7.0 Hz, 3H). LC/MS (ESI) m/z: 576 (M+H)⁺.

### Scheme 126: Synthesis of (8S)—N—((R)-1-(4-carbamimidoylthiophen-2-yl)ethyl)-7-((9-fluoro-9H-fluorene-3-carbonyl)glycyl)-1,4-dioxa-7-azaspiro[4.4]nonane-8-carboxamide (Compound 127)

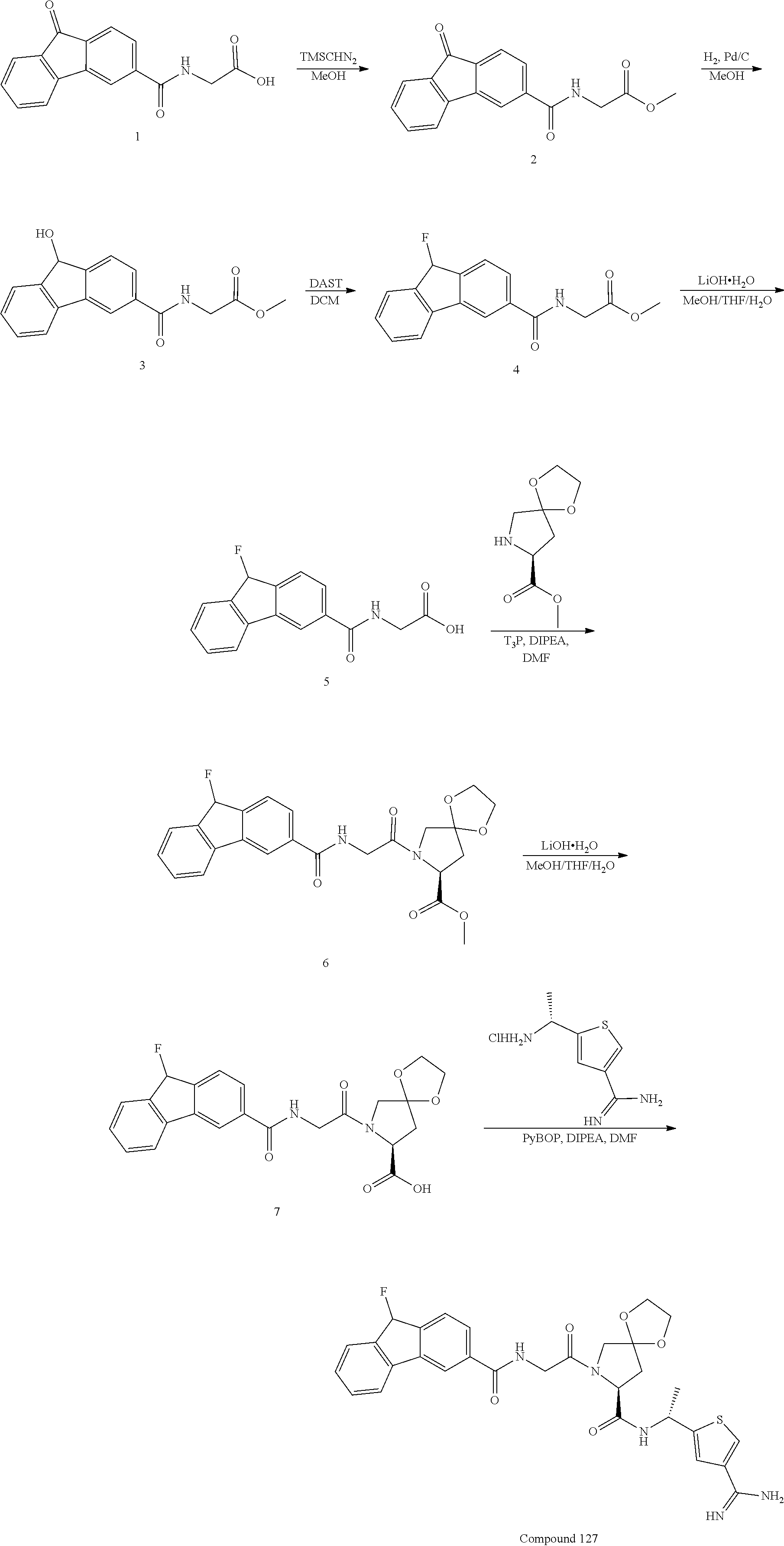

### Step 1: methyl (9-oxo-9H-fluorene-3-carbonyl)glycinate (2)

To a solution of (9-oxo-9H-fluorene-3-carbonyl)glycine (290 mg, 1.03 mmol) in MeOH (2.0 mL) was added TMSCHN₂ (704 mg, 6.12 mmol) at 0° C. and the mixture was stirred at room temperature overnight. The reaction mixture was quenched with ice water and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=1:1) to give methyl (9-oxo-9H-fluorene-3-carbonyl)glycinate (188 mg, yield 61.8%) as a colorless oil. LC/MS (ESI) (m/z): 296 (M+H)⁺.

### Step 2: methyl (9-hydroxy-9H-fluorene-3-carbonyl)glycinate (3)

To a solution of methyl (9-oxo-9H-fluorene-3-carbonyl)glycinate (150 mg, 0.51 mmol) in EtOAc (3 mL) was added Pd/C (15 mg, 10% wt.) at room temperature and the mixture was stirred under H₂ atmosphere at room temperature for 30 minutes. The mixture was filtered, and the filtrate was concentrated to dryness under reduced pressure to give methyl (9-hydroxy-9H-fluorene-3-carbonyl)glycinate (95 mg, yield 62.5%) as a colorless oil, which was used directly in the next step. LC/MS (ESI) (m/z): 298 (M+H)⁺.

### Step 3: methyl (9-fluoro-9H-fluorene-3-carbonyl)glycinate (4)

To a solution of methyl (9-hydroxy-9H-fluorene-3-carbonyl)glycinate (85 mg, 0.29 mmol) in dry DCM (1 mL) was added DAST (70 mg, 0.44 mmol) at 0° C. and the mixture was stirred under N₂ atmosphere at 0° C. for 0.5 hours. The organic layer was diluted with water and extracted with DCM twice. The combined organic layers were washed with brine, dried over anhydrous Na₂SO₄, filtered, and concentrated to dryness under reduced pressure. The residue was purified by flash chromatography on silica gel (PE:EtOAc=1:1) to give methyl (9-fluoro-9H-fluorene-3-carbonyl)glycinate (20 mg, yield 23.0%) as a yellow oil. LC/MS (ESI) (m/z): 300 (M+H)⁺.

### Step 4: (9-fluoro-9H-fluorene-3-carbonyl)glycine (5)

To a solution of methyl (9-fluoro-9H-fluorene-3-carbonyl)glycinate (20 mg, 0.067 mmol) in MeOH (0.75 mL) and water (0.25 mL) was added LiOH·H₂O (6 mg, 0.13 mmol) at 0° C. and the mixture was stirred at room temperature for 1 hour. The mixture was acidified with 1 N aq·HCl to pH-4, extracted with EtOAc twice. The combined organic layers were washed with brine, dried over anhydrous Na₂SO₄, filtered, and concentrated to dryness under reduced pressure to give (9-fluoro-9H-fluorene-3-carbonyl)glycine (16 mg, yield 84.2%) as a colorless oil, which was used directly in the next step. LCMS (ESI) (m/z): 286 (M+H)⁺.

### Step 5: methyl (8S)-7-((9-fluoro-9H-fluorene-3-carbonyl)glycyl)-1,4-dioxa-7-azaspiro[4.4]nonane-8-carboxylate (6)

To a mixture of (9-fluoro-9H-fluorene-3-carbonyl)glycine (16 mg, 0.056 mmol) and methyl (S)-1,4-dioxa-7-azaspiro[4.4]nonane-8-carboxylate (21 mg, 0.11 mmol) in DMF (1.0 mL) was added DIPEA (22 mg, 0.17 mmol) and T₃P (54 mg, 0.084 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography on silica gel (PE:EtOAc=1:3) to give methyl (8S)-7-((9-fluoro-9H-fluorene-3-carbonyl)glycyl)-1,4-dioxa-7-azaspiro[4.4]nonane-8-carboxylate (15 mg, yield 60.0%) as a colorless oil. LC/MS (ESI) m/z: 455 (M+H)⁺.

### Step 6: (6S)-7-((9-fluoro-9H-fluorene-3-carbonyl)glycyl)-1,4-dioxa-7-azaspiro[4.4]nonane-6-carboxylic acid (7)

To a solution of methyl (8S)-7-((9-fluoro-9H-fluorene-3-carbonyl)glycyl)-1,4-dioxa-7-azaspiro[4.4]nonane-8-carboxylate (15 mg, 0.033 mmol) in MeOH (0.75 mL) and water (0.25 mL) was added LiOH·H₂O (3 mg, 0.066 mmol) at 0° C. and the mixture was stirred at room temperature for 1 hour. The mixture was acidified with 1 N aq·HCl to pH˜4, extracted with EtOAc twice. The combined organic layers were washed with brine, dried over anhydrous Na₂SO₄, filtered, and concentrated to dryness under reduced pressure to give (8S)-7-((9-fluoro-9H-fluorene-3-carbonyl)glycyl)-1,4-dioxa-7-azaspiro[4.4]nonane-8-carboxylic acid (12 mg, yield 89.7%) as a colorless oil, which was used directly in the next step. LCMS (ESI) (m/z): 441 (M+H)⁺.

### Step 7: (8S)—N—((R)-1-(4-carbamimidoylthiophen-2-yl)ethyl)-7-((9-fluoro-9H-fluorene-3-carbonyl)glycyl)-1,4-dioxa-7-azaspiro[4.4]nonane-6-carboxamide(Compound 127)

To a mixture of (8S)-7-((9-fluoro-9H-fluorene-3-carbonyl)glycyl)-1,4-dioxa-7-azaspiro[4.4]nonane-8-carboxylic acid (13 mg, 0.029 mmol) and (R)-5-(1-aminoethyl)thiophene-3-carboximidamide hydrochloride (10 mg, 0.059 mmol) in DMF (1 mL) was added DIPEA (19 mg, 0.15 mmol) and PyBOP (17 mg, 0.072 mmol) at 0° C. and the mixture was stirred under N₂ atmosphere at room temperature for 1 hour. The reaction mixture was diluted with water and extracted with EtOAc twice. The combined organic layers were washed with brine, dried over anhydrous Na₂SO₄, filtered, and concentrated to dryness under reduced pressure. The residue was purified by prep-TLC (DCM:MeOH=10:1) and prep-HPLC to give Compound 127 (1.0 mg, yield 5.9%) as a white solid. ¹H NMR (400 MHz, CD₃OD) δ 8.54 (s, 1H), 8.23-8.19 (m, 2H), 7.84 (d, J=8.1 Hz, 1H), 7.75 (dd, J=16.6, 8.4 Hz, 2H), 7.66 (d, J=7.7 Hz, 1H), 7.53-7.47 (m, 2H), 7.40 (t, J=7.3 Hz, 1H), 6.40 (d, J=53.2 Hz, 1H), 5.29 (d, J=6.9 Hz, 1H), 4.58 (t, J=7.7 Hz, 1H), 4.21 (d, J=4.2 Hz, 2H), 4.02-3.95 (m, 4H), 3.81 (d, J=7.1 Hz, 2H), 2.44 (dd, J=13.6, 8.8 Hz, 1H), 2.22 (dd, J=13.0, 6.7 Hz, 1H), 1.61 (t, J=18.8 Hz, 3H). LCMS (ESI) (m/z): 592 (M+H)⁺.

### Scheme 127: Synthesis of (S)-7-((9,9-difluoro-9H-fluorene-3-carbonyl)glycyl)-N—((R)-1-(4-(N-hydroxycarbamimidoyl)thiophen-2-yl)ethyl)-1,4-dioxa-7-azaspiro[4.4]nonane-8-carboxamide (Compound 128)

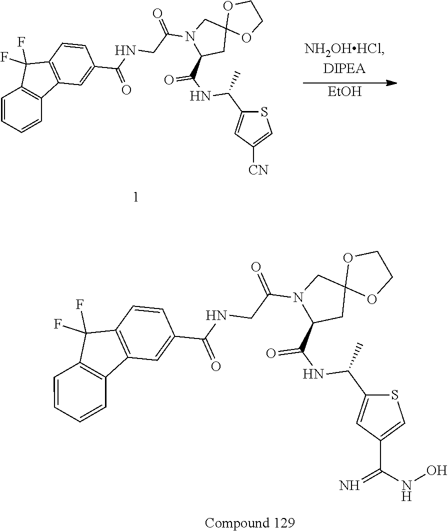 [SMILES: "EtOH" ; C[C@@H](NC(=O)[C@@H]1CC2(CN1C(=O)CNC(=O)c1ccc3c(c1)-c1ccccc1C3(F)F)OCCO2)c1cc(C#N)cs1 ; C[C@@H](NC(=O)[C@@H]1CC2(CN1C(=O)CNC(=O)c1ccc3c(c1)-c1ccccc1C3(F)F)OCCO2)c1cc(C(=N)NO)cs1]

Compound 128 was prepared as a white solid from (S)—N—((R)-1-(4-cyanothiophen-2-yl)ethyl)-7-((9,9-difluoro-9H-fluorene-3-carbonyl)glycyl)-1,4-dioxa-7-azaspiro[4.4]nonane-8-carboxamide and NH₂OH·HCl based on the procedures set forth in Scheme 103. ¹H NMR (400 MHz, CD₃OD) δ 8.19 (s, 1H), 7.88 (d, J=7.7 Hz, 1H), 7.78 (t, J=7.4 Hz, 1H), 7.71 (d, J=7.7 Hz, 1H), 7.64 (d, J=7.5 Hz, 1H), 7.58 (dd, J=9.3, 4.3 Hz, 2H), 7.44 (t, J=7.5 Hz, 1H), 7.35 (d, J=13.2 Hz, 1H), 5.27 (q, J=6.9 Hz, 1H), 4.58 (dd, J=8.9, 5.9 Hz, 1H), 4.22 (dd, J=16.1, 4.4 Hz, 2H), 4.02-3.96 (m, 4H), 3.82-3.75 (m, 2H), 2.43 (dd, J=13.2, 9.1 Hz, 1H), 2.22 (dd, J=13.2, 5.8 Hz, 1H), 1.58 (dd, J=35.8, 6.9 Hz, 3H). LCMS (ESI) (m/z): 626 (M+H)⁺.

### Scheme 128: Synthesis of (8S)-7-{2-[(9,9-difluorofluoren-3-yl)formamido]acetyl}-N-[(1R)-1-[4-(1,3-oxazol-4-yl)thiophen-2-yl]ethyl]-1,4-dioxa-7-azaspiro[4.4]nonane-8-carboxamide (Compound 129)

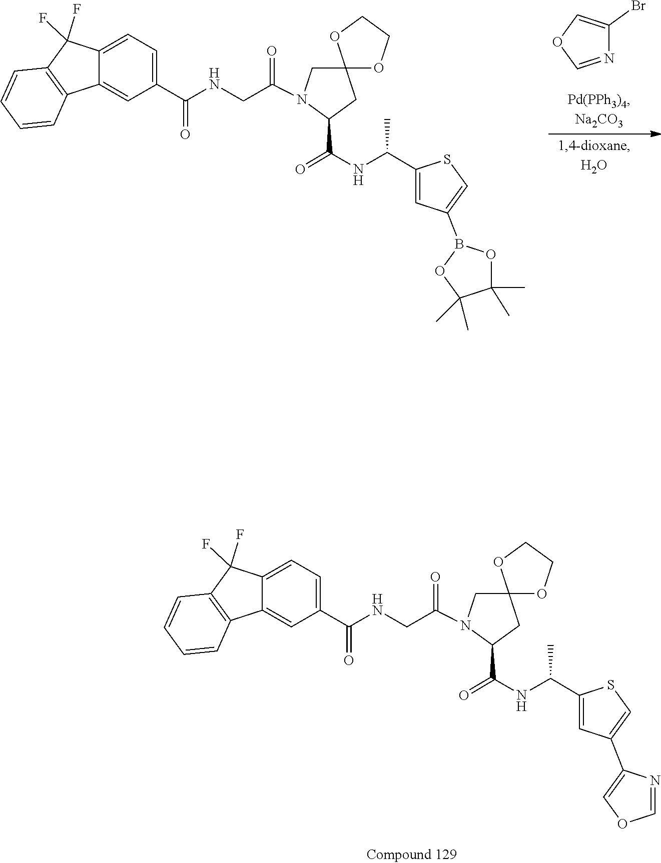 [SMILES: C[C@@H](NC(=O)[C@@H]1CC2(CN1C(=O)CNC(=O)c1ccc3c(c1)-c1ccccc1C3(F)F)OCCO2)c1cc(B2OC(C)(C)C(C)(C)O2)cs1 ; Brc1cocn1 ; C[C@@H](NC(=O)[C@@H]1CC2(CN1C(=O)CNC(=O)c1ccc3c(c1)-c1ccccc1C3(F)F)OCCO2)c1cc(-c2cocn2)cs1]

Compound 129 was prepared as a white solid from (8S)-7-{2-[(9,9-difluorofluoren-3-yl)formamido]acetyl}-N-[(1R)-1-[4-(4,4,5,5-tetramethyl-1,3,2-dioxaborolan-2-yl)thiophen-2-yl]ethyl]-1,4-dioxa-7-azaspiro[4.4]nonane-8-carboxamide and 4-bromo-1,3-oxazole based on the procedures set forth in Scheme 113. ¹H NMR (400 MHz, CD₃OD) δ 8.16 (d, J=2.3 Hz, 1H), 8.13 (d, J=7.0 Hz, 1H), 8.05 (s, 1H), 7.85 (t, J=8.7 Hz, 1H), 7.75-7.61 (m, 3H), 7.54 (dd, J=13.9, 6.2 Hz, 2H), 7.46-7.37 (m, 2H), 5.28 (q, J=6.8 Hz, 1H), 4.62-4.56 (m, 1H), 4.27 (d, J=16.7 Hz, 1H), 4.15 (d, J=16.7 Hz, 1H), 4.04-3.95 (m, 4H), 3.79 (s, 2H), 2.43 (dd, J=13.1, 8.8 Hz, 1H), 2.23 (dd, J=13.2, 6.8 Hz, 1H), 1.59 (t, J=17.8 Hz, 3H). LC/MS (ESI) m/z: 635 (M+H)⁺.

### Scheme 129: Synthesis of (S)—N—((R)-1-(4-(4H-1,2,4-triazol-3-yl)thiophen-2-yl)ethyl)-7-((9,9-difluoro-9H-fluorene-3-carbonyl)glycyl)-1,4-dioxa-7-azaspiro[4.4]nonane-8-carboxamide (Compound 130)

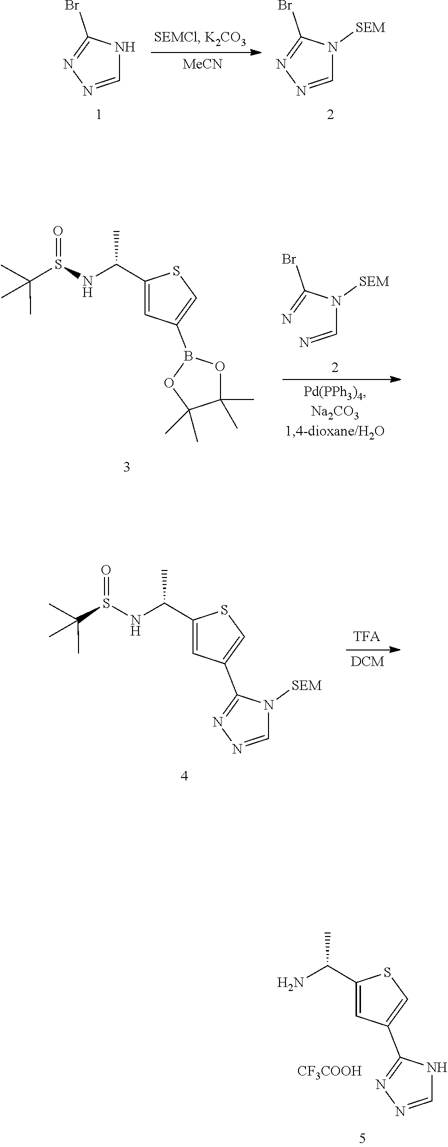

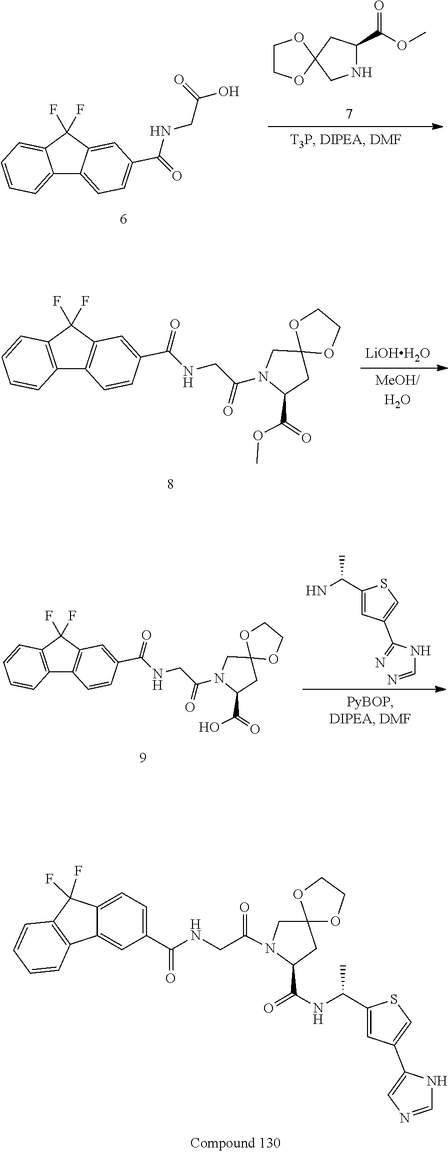

### Step 1: Step 1: 3-bromo-4-((2-(trimethylsilyl)ethoxy)methyl)-4H-1,2,4-triazole (2)

To a solution of 3-bromo-4H-1,2,4-triazole (260 mg, 1.76 mmol) in M7N (5 ml) was added K₂CO₃ (485 mg, 3.51 mmol) and SEMCl (440 mg, 2.64 mmol) at 000. The mixture was stirred at 0 under N₂ atmosphere for 4 hours. The mixture was diluted with water and extracted with EtOAc (3×40 mL). The combined organic layers were washed with brine, dried over anhydrous Na₂SO₄, filtered, and concentrated to dryness under reduced pressure. The residue was purified by flash chromatography on silica gel (PE:EtOAc=10:1) to give 3-bromo-4-((2-(trimethylsilyl)ethoxy)methyl)-4H-1,2,4-triazole (300 mg, yield 61.5%) as a yellow oil. LCMS (ESI) (m/z): 278 (M+H)⁺.

### Step 2: (S)-2-methyl-N—((R)-1-(4-(4-((2-(trimethylsilyl)ethoxy)methyl)-4H-1,2,4-triazol-3-yl) thiophen-2-yl)ethyl)propane-2-sulfinamide (4)

To mixture of (S)-2-methyl-N—((R)-1-(4-(4,4,5,5-tetramethyl-1,3,2-dioxaborolan-2-yl)thiophen-2-yl)ethyl)propane-2-sulfinamide (70 mg, 0.19 mmol) and 3-bromo-4-((2-(trimethylsilyl) ethoxy) methyl)-4H-1,2,4-triazole (109 mg, 0.39 mmol) in dioxane (1.5 mL), water (0.5 ml-) was added Pd(PPh₃)₄(12 mg, 0.097 mmol) and Na₂CO₃ (63 mg, 0.59 mmol). The reaction mixture was stirred under N₂ atmosphere at 85° C. for 4 hours. The mixture was diluted with water and extracted with EtOAc twice. The combined organic layers were washed with brine, dried over anhydrous Na₂SO₄, filtered, and concentrated under reduced pressure to dryness. The residue was purified by column chromatography on silica gel (PE:EtOAc=5:1) to give (S)-2-methyl-N—((R)-1-(4-(4-((2-(trimethylsilyl)ethoxy)methyl)-4H-1,2,4-triazol-3-yl)thiophen-2-yl)ethyl) propane-2-sulfinamide (49 mg, yield 58.5%) as a yellow solid. LCMS (ESI) m/z=429 (M+H)⁺.

### Step 3: (R)-1-(4-(4H-1,2,4-triazol-3-yl)thiophen-2-yl)ethan-1-amine 2,2,2-trifluoroacetate (5)

To a solution of (S)-2-methyl-N—((R)-1-(4-(4-((2-(trimethylsilyl)ethoxy)methyl)-4H-1,2,4-triazol-3-yl)thiophen-2-yl)ethyl)propane-2-sulfinamide (49 mg, 0.11 mmol) in DCM (0.5 mL) was added TFA (0.5 mL) at room temperature and the mixture was stirred overnight. The reaction mixture was concentrated to give (R)-1-(4-(4H-1,2,4-triazol-3-yl)thiophen-2-yl) ethan-1-amine 2,2,2-trifluoroacetate (23 mg, yield 67.6%) as a brown solid, which was used directly in next step without further purification. LC/MS (ESI) m/z:195 (M+H)⁺.

### Step 4: methyl (S)-7-((9,9-difluoro-9H-fluorene-2-carbonyl)glycyl)-1,4-dioxa-7-azaspiro[4.4]nonane-8-carboxylate (8)

To mixture of [(9,9-difluorofluoren-2-yl)formamido]acetic acid (61 mg, 0.2 mmol), methyl (S)-1,4-dioxa-7-azaspiro[4.4]nonane-8-carboxylate (45 mg, 0.24 mmol) and DIPEA (78 mg, 0.6 mmol) in DMF (0.5 mL) was added T₃P (191 mg, 0.3 mmol, 50% in EtOAc) under N₂ atmosphere and the reaction mixture was stirred at room temperature for 1 hour. The mixture was diluted with EtOAc and washed with saturated aq·NaHCO₃ solution. The aqueous layer was extracted with EtOAc twice and the combined organic layers were washed with brine, dried over anhydrous Na₂SO₄, filtered, and concentrated under reduced pressure to dryness. The residue was purified by flash chromatography on silica gel (PE:EtOAc=3:1) to afford methyl (S)-7-((9,9-difluoro-9H-fluorene-2-carbonyl)glycyl)-1,4-dioxa-7-azaspiro[4.4]nonane-8-carboxylate (68 mg, yield 72%) as a yellow oil. LC/MS (ESI) m/z: 473 (M+H)⁺.

### Step 5: (S)-7-((9,9-difluoro-9H-fluorene-2-carbonyl)glycyl)-1,4-dioxa-7-azaspiro[4.4]nonane-8-carboxylic acid (9)

To a solution of methyl (S)-7-((9,9-difluoro-9H-fluorene-2-carbonyl)glycyl)-1,4-dioxa-7-azaspiro[4.4]nonane-8-carboxylate (68 mg, 0.14 mmol) in MeOH (0.8 mL) and H₂O (0.2 mL) was added LiOH (14 mg, 0.33 mmol) under N₂ atmosphere and the reaction mixture was stirred at room temperature for 3 hours. The mixture was acidified with 1 N aq·HCl to pH-3, extracted with EtOAc twice. The combined organic layers were washed with brine, dried over anhydrous Na₂SO₄, filtered, and concentrated under reduced pressure to give (S)-7-((9,9-difluoro-9H-fluorene-2-carbonyl)glycyl)-1,4-dioxa-7-azaspiro[4.4]nonane-8-carboxylic acid (56 mg, yield 85%) as a white solid, which was used in next step without purification LC/MS (ESI) m/z:459 (M+H)⁺.

### Step 6: (S)—N—((R)-1-(4-(4H-1,2,4-triazol-3-yl)thiophen-2-yl)ethyl)-7-((9,9-difluoro-9H-fluorene-3-carbonyl)glycyl)-1,4-dioxa-7-azaspiro[4.4]nonane-8-carboxamide (Compound 130)

To a mixture of (R)-1-(4-(4H-1,2,4-triazol-3-yl)thiophen-2-yl)ethan-1-amine (5 mg, 0.026 mmol) and (S)-7-((9,9-difluoro-9H-fluorene-3-carbonyl)glycyl)-1,4-dioxa-7-azaspiro[4.4]nonane-8-carboxylic acid (12 mg, 0.026 mmol) in DMF (0.5 mL) was added DIPEA (20 mg, 0.16 mmol) and PyBOP (14 mg, 0.026 mmol) at 0° C. and the mixture was stirred under N₂ atmosphere at room temperature for 1 hour. The reaction mixture was diluted with water and extracted with EtOAc twice. The combined organic layers were washed with brine, dried over anhydrous Na₂SO₄, filtered, and concentrated to dryness under reduced pressure. The residue was purified by flash chromatography on silica gel (DCM:MeOH=8:1) and prep-HPLC to give Compound 130 (0.9 mg, yield 5.5%) as a white solid. ¹H NMR (400 MHz, CD₃OD) δ 8.49 (s, 1H), 8.17 (s, 1H), 7.87 (d, J=7.9 Hz, 2H), 7.75-7.63 (m, 3H), 7.57 (d, J=7.8 Hz, 2H), 7.48-7.33 (m, 2H), 5.33 (d, J=6.6 Hz, 1H), 4.61 (d, J=6.0 Hz, 1H), 4.20 (s, 2H), 4.00 (s, 4H), 3.80 (s, 2H), 2.28-2.17 (m, 2H), 1.59 (d, J=6.9 Hz, 3H). LCMS (ESI) (m/z): 635 (M+H)⁺.

### Scheme 130: Synthesis of (2S,4R)—N—((R)-1-(4-carbamimidoylthiophen-2-yl)ethyl)-1-((4-(difluoro(phenyl)methyl)benzoyl)glycyl)-4-(difluoromethoxy)pyrrolidine-2-carboxamide (Compound 131)

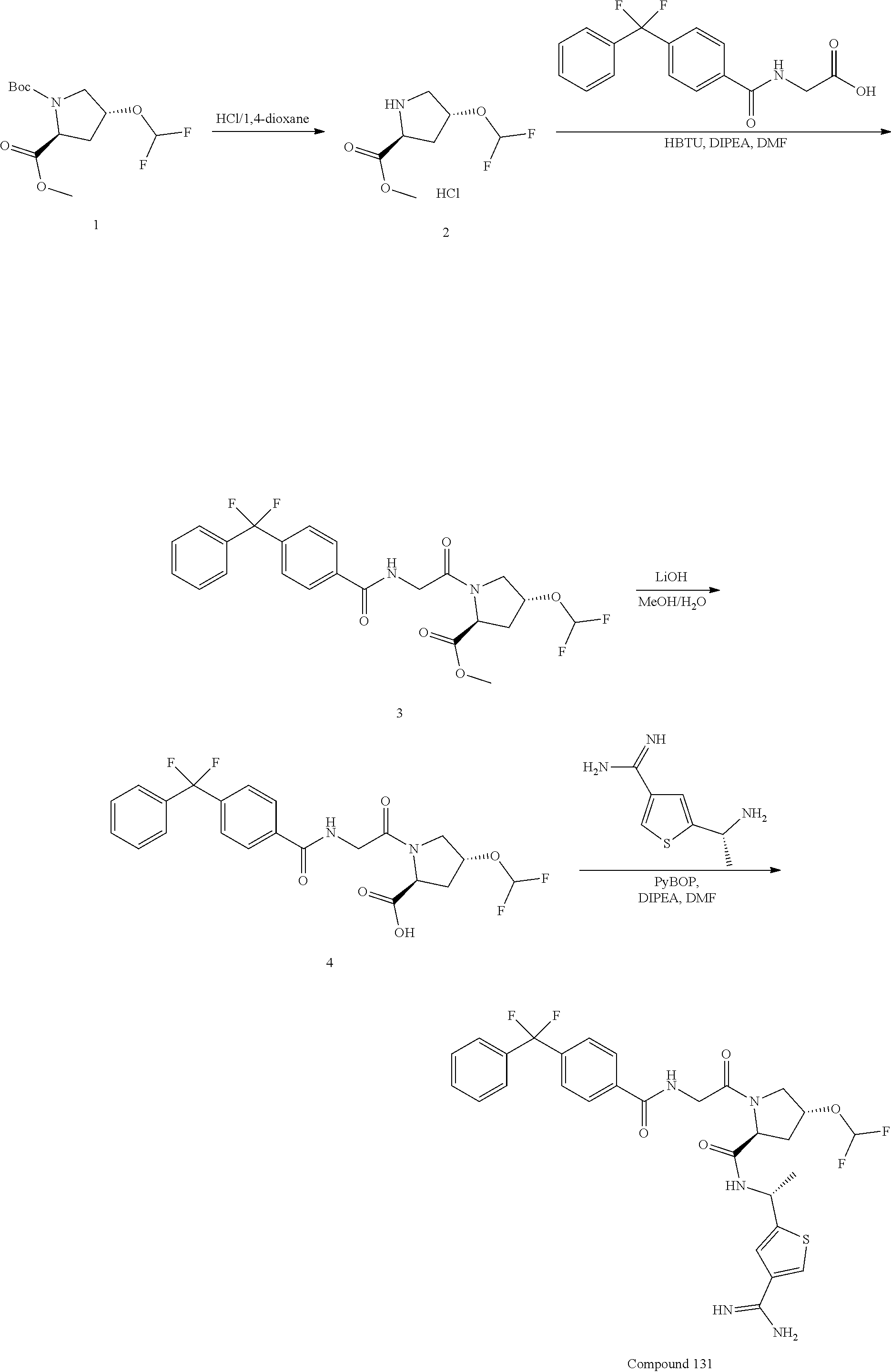

Compound 131 was prepared as a white solid from 1-(tert-butyl) 2-methyl (2S,4R)-4-(difluoromethoxy)pyrrolidine-1,2-dicarboxylate, (4-(difluoro(phenyl)methyl)benzoyl)glycine, and (R)-5-(1-aminoethyl) thiophene-3-carboximidamide based on the procedures set forth in Scheme 3. ¹H NMR (400 MHz, CD₃OD) δ 8.53 (s, 1H), 8.22 (dd, J=7.9, 1.5 Hz, 1H), 7.92 (t, J=8.7 Hz, 2H), 7.62 (d, J=8.3 Hz, 2H), 7.50 (d, J=3.9 Hz, 3H), 7.46 (dd, J=5.2, 1.9 Hz, 3H), 6.51 (t, J=74.5 Hz, 1H), 5.28 (dd, J=21.5, 15.1 Hz, 1H), 4.98 (d, J=27.1 Hz, 1H), 4.56 (d, J=8.0 Hz, 1H), 4.28 (d, J=16.7 Hz, 1H), 4.16 (d, J=16.7 Hz, 1H), 3.95 (dd, J=11.5, 4.6 Hz, 1H), 3.87 (d, J=11.5 Hz, 1H), 2.52-2.43 (m, 1H), 2.25-2.18 (m, 1H), 1.58 (d, J=7.0 Hz, 3H). LCMS (ESI) (m/z): 620 (M+H)⁺.

### Scheme 131: Synthesis of (2S,4R)—N—((R)-1-(4-carbamimidoylthiophen-2-yl)ethyl)-4-(difluoromethoxy)-1-((9,9-dimethyl-9H-fluorene-3-carbonyl)glycyl)pyrrolidine-2-carboxamide (Compound 132)

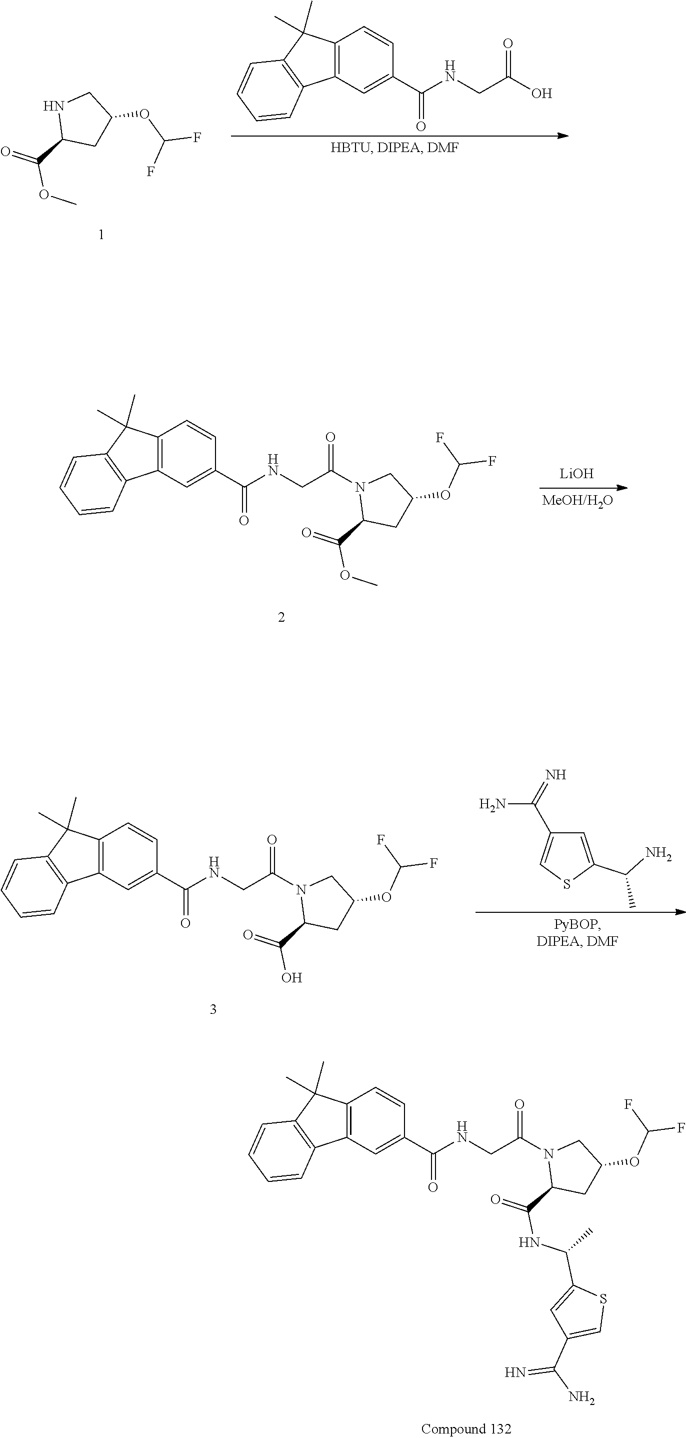

Compound 132 was prepared as a white solid from methyl (2S,4R)-4-(o-tolyl)pyrrolidine-2-carboxylate, (9,9-dimethyl-9H-fluorene-3-carbonyl)glycine, and (R)-5-(1-aminoethyl)thiophene-3-carboximidamide based on the procedures set forth in Scheme 3. ¹H NMR (400 MHz, CD₃OD) δ 8.53 (s, 1H), 8.27-8.20 (m, 2H), 7.81 (ddd, J=12.6, 6.7, 1.9 Hz, 2H), 7.59-7.49 (m, 3H), 7.36 (dd, J=5.2, 3.5 Hz, 2H), 6.51 (td, J=74.4, 16.5 Hz, 1H), 5.36-5.26 (m, 1H), 4.99 (d, J=31.3 Hz, 1H), 4.61 (d, J=7.8 Hz, 1H), 4.31 (d, J=16.7 Hz, 1H), 4.20 (d, J=16.6 Hz, 1H), 4.00-3.89 (m, 2H), 2.49 (dd, J=11.0, 7.2 Hz, 1H), 2.26-2.19 (m, 1H), 1.61 (t, J=15.5 Hz, 3H), 1.49 (s, 6H). LCMS (ESI) (m/z): 610 (M+H)⁺.

### Scheme 132: Synthesis of 18-(((S)-4-((2-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)ethyl)amino)-1-carboxy-4-oxobutyl)amino)-18-oxooctadecanoic acid (Compound 133)

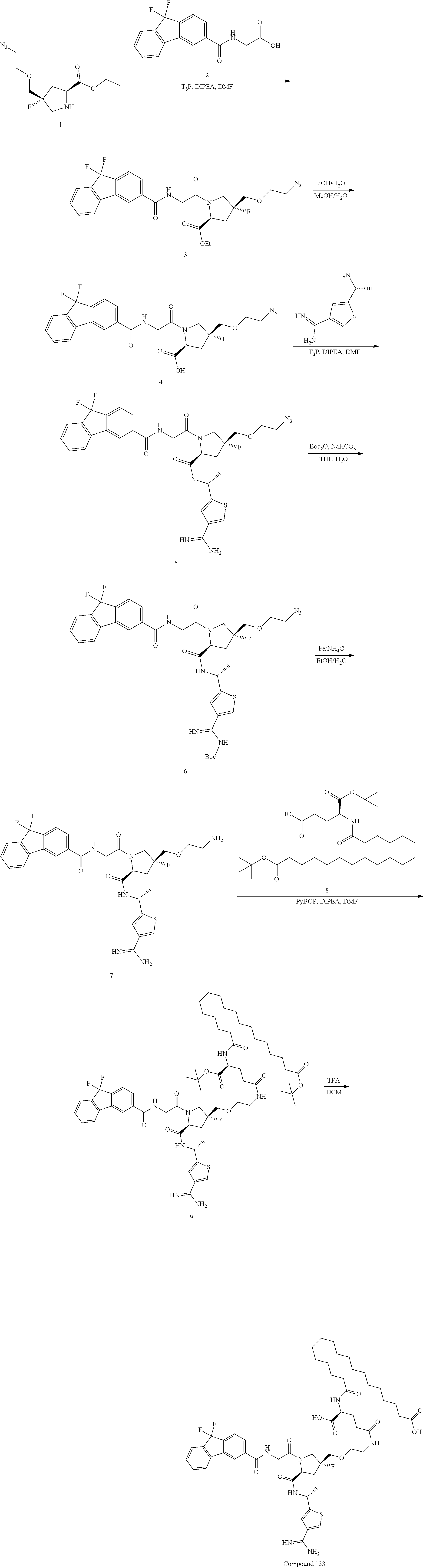

### Step 1: ethyl (2S,4R)-4-((2-azidoethoxy)methyl)-1-((9,9-difluoro-9H-fluorene-3-carbonyl) glycyl)-4-fluoropyrrolidine-2-carboxylate (3)

To a mixture of ethyl (2S,4R)-4-[(2-azidoethoxy)methyl]-4-fluoropyrrolidine-2-carboxylate (590 mg, 2.27 mmol) and [(9,9-difluorofluoren-3-yl)formamido]acetic acid (756 mg, 2.49 mmol) in DMF (8 mL) was added DIPEA (1.98 mL, 11.33 mmol) and T₃P (2.88 g, 4.53 mmol, 50% wt. in EtOAc) under N₂ atmosphere and the reaction mixture was stirred at room temperature for 2 hours. The mixture was diluted with EtOAc, washed with saturated aq·NaHCO₃ and brine, dried over anhydrous Na₂SO₄, filtered, and concentrated under reduced pressure to dryness. The residue was purified by column chromatography on silica gel (PE:EtOAc=1:1) to give ethyl (2S,4R)-4-((2-azidoethoxy)methyl)-1-((9,9-difluoro-9H-fluorene-3-carbonyl)glycyl)-4-fluoropyrrolidine-2-carboxylate (1.23 g, yield 99.5%) as a yellow oil. LC/MS(ESI) m/z: 546 (M+H)⁺.

### Step 2: (2S,4R)-4-((2-azidoethoxy)methyl)-1-((9,9-difluoro-9H-fluorene-3-carbonyl)glycyl)-4-fluoropyrrolidine-2-carboxylic acid (4)

To a solution of ethyl (2S,4R)-4-((2-azidoethoxy)methyl)-1-((9,9-difluoro-9H-fluorene-3-carbonyl)glycyl)-4-fluoropyrrolidine-2-carboxylate (1.23 g, 2.26 mol) in MeOH (8 mL) and THF (4 mL) was added a solution of LiOH·H₂O (42 mg, 1.01 mmol) in water (4 mL) at 0° C. and the mixture was stirred at room temperature for 2 hours. The mixture was acidified with 0.5N aq·HCl to pH˜3 and extracted with EtOAc twice. The combined organic layers were washed with brine, dried over anhydrous Na₂SO₄, filtered, and concentrated under reduced pressure to give (2S,4R)-4-((2-azidoethoxy)methyl)-1-((9,9-difluoro-9H-fluorene-3-carbonyl)glycyl)-4-fluoropyrrolidine-2-carboxylic acid (1.03 g, yield 88.3%) as a yellow solid, which was used directly in the next reaction. LC/MS (ESI) (m/z): 518 (M+H)⁺.

### Step 3: (2S,4R)-4-((2-azidoethoxy)methyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (5)

To a mixture of (2S,4R)-4-((2-azidoethoxy)methyl)-1-((9,9-difluoro-9H-fluorene-3-carbonyl) glycyl)-4-fluoropyrrolidine-2-carboxylic acid (530 mg, 1.02 mmol) and (R)-5-(1-aminoethyl)thiophene-3-carboximidamide (347 mg, 2.05 mmol) in DMF (7 mL) was added DIPEA (0.72 mL, 4.10 mmol) and PyBOP (694 mg, 1.33 mmol) and the reaction mixture was stirred at room temperature for 1 hour. The mixture was concentrated under reduced pressure to give (2S,4R)-4-((2-azidoethoxy)methyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (684 mg, yield 99.9%) as a yellow oil, which was used directly in the next reaction without further purification. LC/MS(ESI) m/z: 669 (M+H)⁺.

### Step 4: tert-butyl ((5-((R)-1-((2S,4R)-4-((2-azidoethoxy)methyl)-1-((9,9-difluoro-9H-fluorene-3-carbonyl)glycyl)-4-fluoropyrrolidine-2-carboxamido)ethyl)thiophen-3-yl)(imino)methyl)carbamate (6)

To a mixture of (2S,4R)-4-((2-azidoethoxy)methyl)-N—((R)-1-(4-carbamimidoylthiophen-2-yl) ethyl)-1-((9,9-difluoro-9H-fluorene-3-carbonyl)glycyl)-4-fluoropyrrolidine-2-carboxamide (680 mg, 1.02 mmol) and di-tert-butyl dicarbonate (666 mg, 3.05 mmol) in THF (9 mL) was added a solution of NaHCO₃ (854 mg, 10.17 mmol) in H₂O (3 mL) at 0° C. and the mixture was stirred at room temperature for 16 hours. The mixture was diluted with water and extracted with EtOAc twice. The combined organic layers were dried over anhydrous Na₂SO₄, filtered, and concentrated to dryness under reduced pressure. The residue was purified by flash chromatography (DCM:MeOH=60:1) to give tert-butyl ((5-((R)-1-((2S,4R)-4-((2-azidoethoxy)methyl)-1-((9,9-difluoro-9H-fluorene-3-carbonyl)glycyl)-4-fluoropyrrolidine-2-carboxamido)ethyl)thiophen-3-yl)(imino)methyl)carbamate (780 mg, yield 99.8%) as a light oil. LC/MS (ESI) m/z: 769(M+H)⁺.

### Step 5: (2S,4R)-4-((2-aminoethoxy)methyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (7)

To a solution of tert-butyl ((5-((R)-1-((2S,4R)-4-((2-azidoethoxy)methyl)-1-((9,9-difluoro-9H-fluorene-3-carbonyl)glycyl)-4-fluoropyrrolidine-2-carboxamido)ethyl)thiophen-3-yl)(imino)methyl)carbamate (750 mg, 0.98 mmol) in EtOH (10 mL) was added iron powder (327 mg, 5.85 mmol) and a solution of NH₄Cl (313 mg, 5.85 mmol) in water (2 mL), and the reaction mixture was stirred at 50° C. under N₂ atmosphere for 7 hours. The mixture was filtered and concentrated to dryness under reduced pressure. The residue was purified by prep-HPLC to give (2S,4R)-4-((2-aminoethoxy)methyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (240 mg, yield 38.3%) as a white solid. LC/MS (ESI) m/z: 643(M+H)⁺.

### Step 6: tert-butyl 18-(((S)-1-(tert-butoxy)-5-((2-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)ethyl)amino)-1,5-dioxopentan-2-yl)amino)-16-oxooctadecanoate (9)

To a mixture of (2S,4R)-4-((2-aminoethoxy)methyl)-N—((R)-1-(4-carbamimidoylthiophen-2-yl) ethyl)-1-((9,9-difluoro-9H-fluorene-3-carbonyl)glycyl)-4-fluoropyrrolidine-2-carboxamide (120 mg, 0.19 mmol) and PyBOP (146 mg, 0.28 mmol) in DMF (5 mL) was added DIPEA (0.20 mL, 1.12 mmol) and (S)-5-(tert-butoxy)-4-(18-(tert-butoxy)-18-oxooctadecanamido)-5-oxopentanoic acid (311 mg, 0.56 mmol) in batches under N₂ atmosphere and the reaction mixture was stirred at room temperature for 4 hours. The mixture was concentrated to dryness under reduced pressure. The residue was purified by prep-HPLC to give tert-butyl 18-(((S)-1-(tert-butoxy)-5-((2-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)ethyl)amino)-1,5-dioxopentan-2-yl)amino)-18-oxooctadecanoate (35 mg, yield 15.9%) as a white solid. LC/MS(ESI) m/z: 1180.6 (M+H)⁺.

### Step 7: 18-(((S)-4-((2-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)ethyl)amino)-1-carboxy-4-oxobutyl)amino)-16-oxooctadecanoic acid (Compound 133)

To a solution of tert-butyl 18-(((S)-1-(tert-butoxy)-5-((2-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)ethyl)amino)-1,5-dioxopentan-2-yl)amino)-18-oxooctadecanoate (35 mg, 0.03 mmol) in DCM (3 mL) was added TFA (1 mL) and the mixture was stirred at room temperature for 3 hours. The reaction mixture was concentrated to dryness under reduced pressure, co-evaporated with DCM and dried under vacuum. The residue was purified by prep-HPLC to give Compound 133 (15 mg, yield 47.4%) as a white solid. ¹H NMR (400 MHz, CD₃OD) δ 8.27-8.18 (m, 2H), 7.91-7.87 (m, 1H), 7.80-7.74 (m, 1H), 7.70 (d, J=7.6 Hz, 1H), 7.65 (d, J=7.2 Hz, 1H), 7.62-7.54 (m, 2H), 7.47-7.42 (m, 1H), 5.38-5.23 (m, 1H), 4.67-4.61 (m, 1H), 4.35-4.26 (m, 2H), 4.19 (d, J=16.7 Hz, 1H), 4.10-3.92 (m, 2H), 3.81-3.72 (m, 2H), 3.62 (t, J=5.2 Hz, 2H), 3.43-3.34 (m, 2H), 2.63-2.49 (m, 1H), 2.32-2.07 (m, 9H), 1.98-1.91 (m, 1H), 1.62-1.56 (m, 6H), 1.31-1.21 (m, 24H). LC/MS (ESI) (m/z): 1068.6 (M+H)⁺.

### Scheme 133: Synthesis of isopropyl ((E)-amino(5-((R)-1-((S)-7-((9,9-difluoro-9H-fluorene-3-carbonyl)glycyl)-1,4-dioxa-7-azaspiro[4.4]nonane-8-carboxamido)ethyl) thiophen-3-yl)methylene)carbamate (Compound 134)

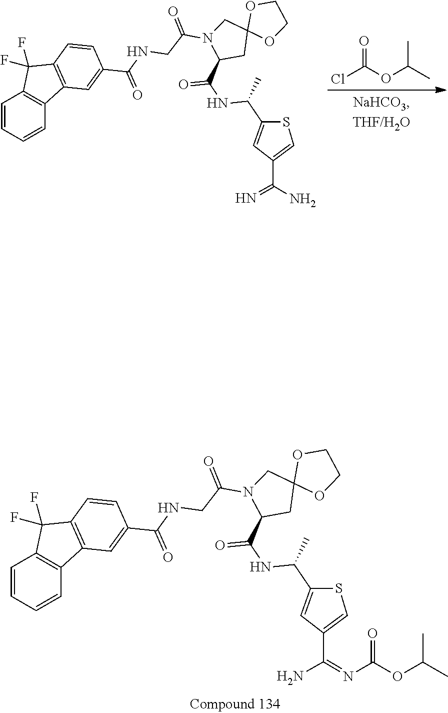

To a mixture of (S)—N—((R)-1-(4-carbamimidoylthiophen-2-yl)ethyl)-7-((9,9-difluoro-9H-fluorene-3-carbonyl)glycyl)-1,4-dioxa-7-azaspiro[4.4]nonane-8-carboxamide (100 mg, 0.16 mmol) and NaHCO₃ (40 mg, 0.48 mmol) in THF (1.5 mL) and water (0.5 mL) was added isopropyl carbonochloridate (30 mg, 0.24 mmol) and the mixture was stirred at room temperature for 10 minutes. The mixture was concentrated to dryness under reduced pressure. The residue was purified by prep-TLC (DCM:MeOH=10:1) and prep-HPLC to give Compound 134 (22.0 mg, yield 19.8%) as a white solid. ¹H NMR (400 MHz, CD₃OD) δ 8.14 (s, 1H), 8.05 (dd, J=19.2, 1.3 Hz, 1H), 7.85 (dd, J=7.8, 1.4 Hz, 1H), 7.69 (dd, J=13.5, 7.8 Hz, 3H), 7.62 (d, J=7.0 Hz, 1H), 7.54 (t, J=7.4 Hz, 1H), 7.42 (t, J=7.5 Hz, 1H), 5.24 (q, J=6.8 Hz, 1H), 4.76-4.71 (m, 1H), 4.59 (s, 1H), 4.28 (d, J=16.7 Hz, 1H), 4.12 (d, J=16.7 Hz, 1H), 3.99 (dd, J=21.0, 2.6 Hz, 4H), 3.80 (d, J=11.2 Hz, 2H), 2.43 (dd, J=13.1, 8.7 Hz, 1H), 2.22 (dd, J=13.1, 7.0 Hz, 1H), 1.59 (dd, J=41.4, 6.9 Hz, 3H), 1.26-1.16 (m, 6H). LC/MS (ESI) m/z: 696 (M+H)⁺.

### Scheme 134: Synthesis of (S)—N—((R)-1-(5-(1H-imidazol-2-yl)thiophen-2-yl)ethyl)-7-((9,9-difluoro-9H-fluorene-3-carbonyl)glycyl)-1,4-dioxa-7-azaspiro[4.4]nonane-8-carboxamide (Compound 135)

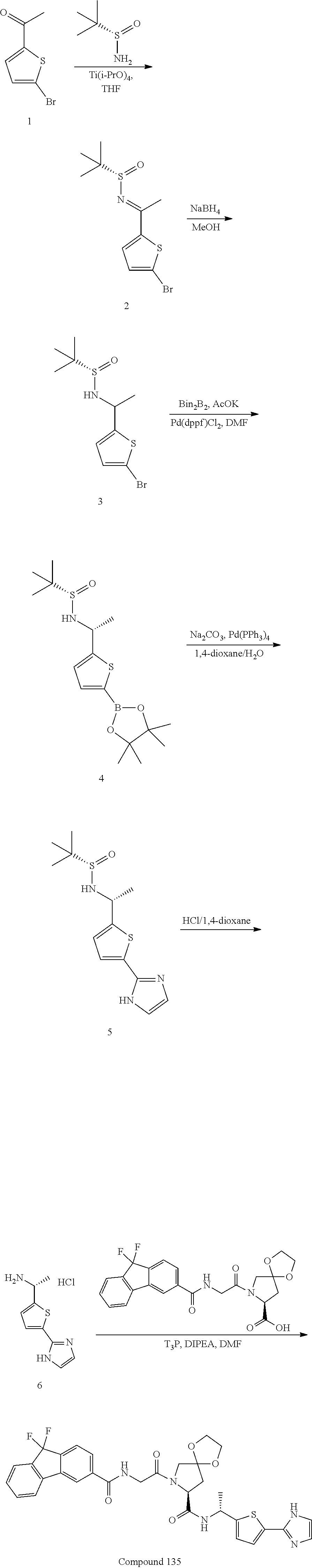

### Step 1: (S,E)-N-(1-(5-bromothiophen-2-yl)ethylidene)-2-methylpropane-2-sulfinamide (2)

To a mixture of 1-(5-bromothiophen-2-yl)ethan-1-one (3.0 g, 14.63 mmol) and (S)-2-methylpropane-2-sulfinamide (5.3 g, 43.89 mmol) in THF (30 mL) was added Ti(i-PrO)₄ (25 g, 87.78 mmol) and the mixture was stirred at 70° C. for 16 hours. The mixture was poured into water and filtered. The filter cake was washed with EA twice and the combined organic layers were washed with brine, dried over anhydrous Na₂SO₄, and concentrated under reduced pressure to dryness. The residue was purified by chromatography on silica gel (PE:EtOAc=9:1) to give (S,E)-N-(1-(5-bromothiophen-2-yl)ethylidene)-2-methylpropane-2-sulfinamide (3.7 g, yield 82.4%) as a brown solid. LC/MS (ESI) (m/z): 308/310 (M+H)⁺.

### Step 2: (S)—N—((R)-1-(5-bromothiophen-2-yl)ethyl)-2-methylpropane-2-sulfinamide (3)

To a solution of (S,E)-N-(1-(5-bromothiophen-2-yl)ethylidene)-2-methylpropane-2-sulfinamide (3.7 g, 12.05 mmol) in MeOH (40 mL) was added NaBH₄ (916 mg, 24.10 mmol) at −30° C. under N₂ atmosphere, and the mixture was then stirred at −30° C. for 0.5 hours. The mixture was quenched with saturated aq·NH₄Cl solution at 0° C. and extracted with DCM twice, dried over anhydrous Na₂SO₄, filtered, and concentrated to dryness. The residue was purified by chromatography on silica gel (PE:EtOAc=4:1) to give (S)—N—((R)-1-(5-bromothiophen-2-yl)ethyl)-2-methylpropane-2- sulfinamide (550 mg, yield 14.8%) as a brown oil. LC/MS (ESI) m/z: 310/312(M+H)⁺.

### Step 3: (S)—N—((R)-1-(5-(1H-imidazol-2-yl)thiophen-2-yl)ethyl)-2-methylpropane-2-sulfinamide (5)

To a mixture of (S)—N—((R)-1-(5-bromothiophen-2-yl)ethyl)-2-methylpropane-2-sulfinamide (500 mg, 1.62 mmol) and Bin₂B₂ (822 mg, 3.24 mmol) in DMF (6 mL) was added KOAc (476 mg, 4.86 mmol) and Pd(dppf)Cl₂ (120 mg, 0.16 mmol) under N₂ atmosphere and the mixture was stirred at 100° C. overnight. Then the mixture was added dioxane (8 mL) and H₂O (2 mL), 2-bromo-1H-imidazole (346 mg, 2.43 mmol), Pd(PPh₃)₄ (94 mg, 0.08 mmol) and Na₂CO₃ (344 mg, 3.24 mmol) under N₂ atmosphere and the mixture was stirred at 100° C. for 2 hours. The mixture was diluted with EtOAc, washed with brine, dried over anhydrous Na₂SO₄, filtered, and concentrated under reduced pressure to dryness. The residue was purified by flash chromatography on silica gel (DCM:MeOH=20:1) to give (S)—N—((R)-1-(5-(1H-imidazol-2-yl)thiophen-2-yl)ethyl)-2-methylpropane-2-sulfinamide (120 mg, yield 22.8%) as a brown oil. LC/MS (ESI) m/z: 298 (M+H)⁺.

### Step 4: (R)-1-(5-(1H-imidazol-2-yl)thiophen-2-yl)ethan-1-amine hydrochloride (6)

A solution of (S)—N—((R)-1-(5-(1H-imidazol-2-yl)thiophen-2-yl)ethyl)-2-methylpropane-2-sulfinamide (120 mg, 0.40 mmol) in HCl/1,4-dioxane (5 mL) was stirred at room temperature for 1 hour. The mixture was concentrated to dryness under reduced pressure to give (R)-1-(5-(1H-imidazol-2-yl)thiophen-2-yl)ethan-1-amine hydrochloride (60 mg, yield 65.1%) as a brown oil, which was directly used in the next reaction without further purification. LC/MS (ESI) m/z: 194 (M+H)⁺.

### Step 5: (S)—N—((R)-1-(5-(1H-imidazol-2-yl)thiophen-2-yl)ethyl)-7-((9,9-difluoro-9H-fluorene-3-carbonyl)glycyl)-1,4-dioxa-7-azaspiro[4.4]nonane-6-carboxamide (Compound 135)

To a mixture of (S)-7-((9,9-difluoro-9H-fluorene-3-carbonyl)glycyl)-1,4-dioxa-7-azaspiro[4.4]nonane-8-carboxylic acid (50 mg, 0.11 mmol) and (R)-1-(5-(1H-imidazol-2-yl)thiophen-2-yl)ethan-1-amine (63 mg, 0.33 mmol) in DMF (3 mL) was added DIPEA (0.13 mL, 0.66 mmol) and T₃P (208 mg, 0.33 mmol, 50% wt. in EtOAc) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by column chromatography on silica gel (DCM:MeOH=10:1) to give Compound 135 (20 mg, yield 29.0%) as a white solid. ¹H NMR (400 MHz, CD₃OD) δ 8.12 (d, J=33.3 Hz, 1H), 7.86-7.79 (m, 1H), 7.70-7.60 (m, 3H), 7.52 (t, J=7.6 Hz, 1H), 7.42 (t, J=7.5 Hz, 1H), 7.25 (dd, J=39.4, 3.7 Hz, 1H), 7.03-6.92 (m, 3H), 5.39-5.30 (m, 1H), 4.61 (dd, J=8.7, 6.1 Hz, 1H), 4.25-4.14 (m, 2H), 4.02 (s, 3H), 3.94 (d, J=16.5 Hz, 1H), 3.84 (q, J=10.7 Hz, 2H), 2.45 (dd, J=12.8, 9.3 Hz, 1H), 2.24 (dd, J=13.1, 5.9 Hz, 1H), 1.64 (d, J=6.9 Hz, 3H). LC/MS (ESI) m/z: 634 (M+H)⁺.

### Scheme 135: Synthesis of (2S,4R)—N—((R)-1-(4-carbamimidoylthiophen-2-yl)ethyl)-1-((4-(difluoro(phenyl)methyl)benzoyl)glycyl)-4-fluoro-4-(methoxymethyl)pyrrolidine-2-carboxamide (Compound 136)

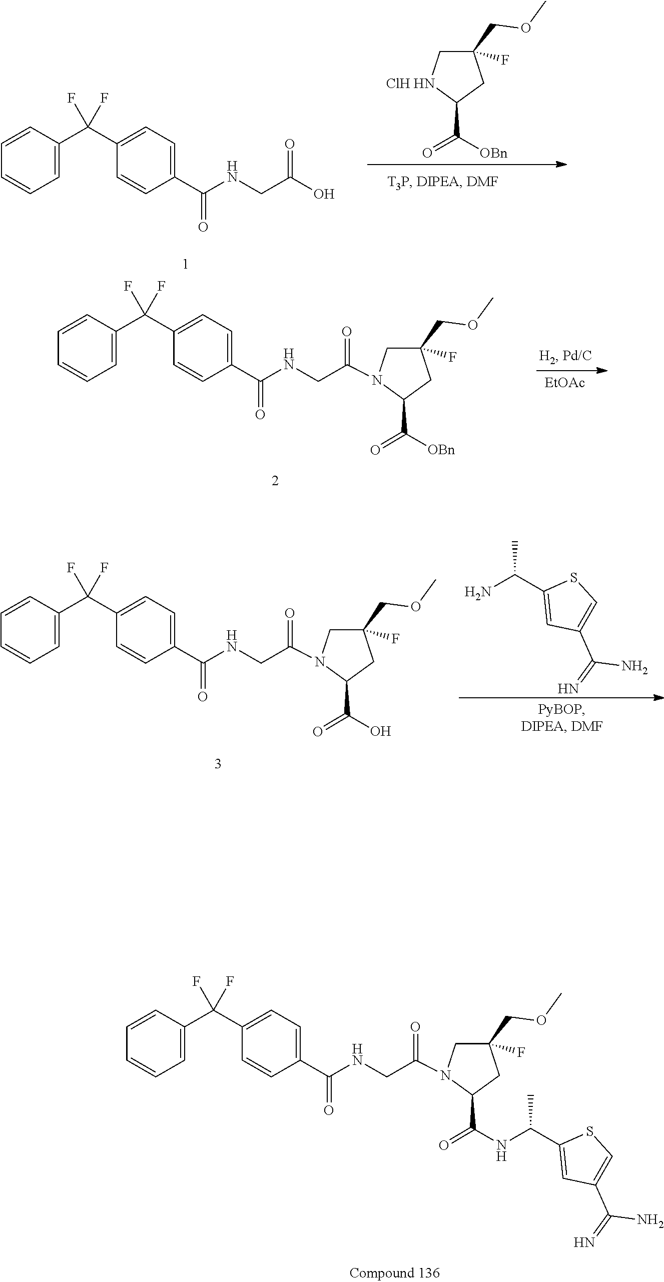

Compound 136 was prepared as a white solid from (4-(difluoro(phenyl)methyl)benzoyl)glycine, benzyl (2S,4R)-4-fluoro-4-(methoxymethyl)pyrrolidine-2-carboxylate hydrochloride, and (R)-5-(1-aminoethyl)thiophene-3-carboximidamide based on the procedures set forth in Scheme 12. ¹H NMR (400 MHz, CD₃OD) δ 8.26-8.19 (m, 1H), 7.95-7.90 (m, 2H), 7.62 (d, J=8.4 Hz, 2H), 7.55-7.49 (m, 3H), 7.49-7.44 (m, 3H), 5.40-5.22 (m, 1H), 4.65-4.57 (m, 1H), 4.31 (d, J=16.8 Hz, 1H), 4.16-4.10 (m, 1H), 4.09-4.00 (m, 1H), 3.95-3.83 (m, 1H), 3.73-3.64 (m, 2H), 3.42 (d, J=5.2 Hz, 3H), 2.61-2.46 (m, 1H), 2.26-2.10 (m, 1H), 1.66-1.56 (m, 3H). LC/MS (ESI) m/z: 616 (M+H)⁺.

### Scheme 136: Synthesis of (S)—N—((R)-1-(4-carbamimidoylthiophen-2-yl)ethyl)-7-((dibenzo[b,d]furan-2-carbonyl)glycyl)-1,4-dioxa-7-azaspiro[4.4]nonane-8-carboxamide (Compound 137)

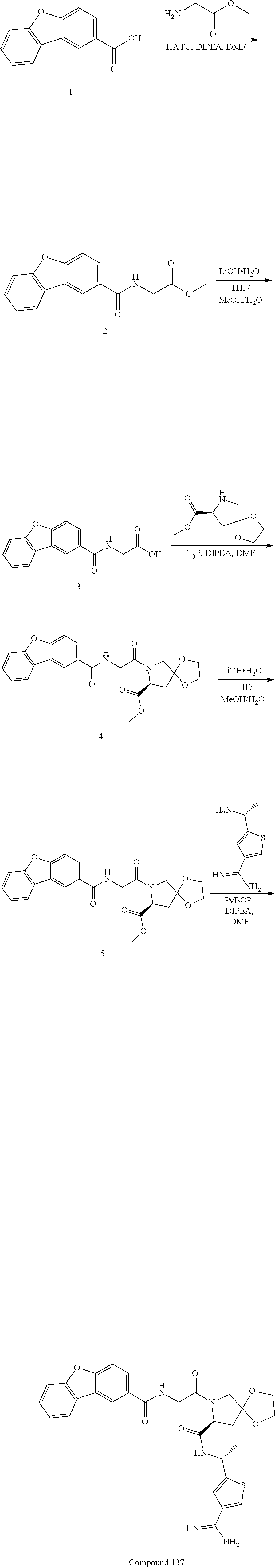

Compound 137 was prepared as a white solid from dibenzo[b,d]furan-2-carboxylic acid, methyl glycinate, methyl (S)-1,4-dioxa-7-azaspiro[4.4]nonane-8-carboxylate, and (R)-5-(1-aminoethyl)thiophene-3-carboximidamide based on the procedures set forth in Scheme 7. ¹H NMR (400 MHz, CD₃OD) δ 8.58 (d, J=1.6 Hz, 1H), 8.53 (s, 1H), 8.23 (dd, J=18.3, 1.5 Hz, 1H), 8.08 (t, J=8.9 Hz, 1H), 8.01 (dd, J=8.6, 1.8 Hz, 1H), 7.65 (dd, J=10.9, 7.1 Hz, 2H), 7.54 (dd, J=13.1, 4.2 Hz, 2H), 7.42 (t, J=7.5 Hz, 1H), 5.41-5.26 (m, 1H), 4.59 (dd, J=8.7, 6.7 Hz, 1H), 4.27-4.14 (m, 2H), 4.00 (qd, J=7.6, 4.6 Hz, 4H), 3.86-3.77 (m, 2H), 2.53 (ddd, J=63.5, 12.9, 9.1 Hz, 1H), 2.38-2.18 (m, 1H), 1.61 (t, J=19.9 Hz, 3H). LC/MS (ESI) m/z:576 (M+H)⁺.

### Scheme 137: Synthesis of (S)—N—((R)-1-(4-carbamimidoylthiophen-2-yl)ethyl)-7-((9,9-difluoro-9H-fluorene-7-carbonyl)glycyl)-1,4-dioxa-7-azaspiro[4.4]nonane-8-carboxamide (Compound 138)

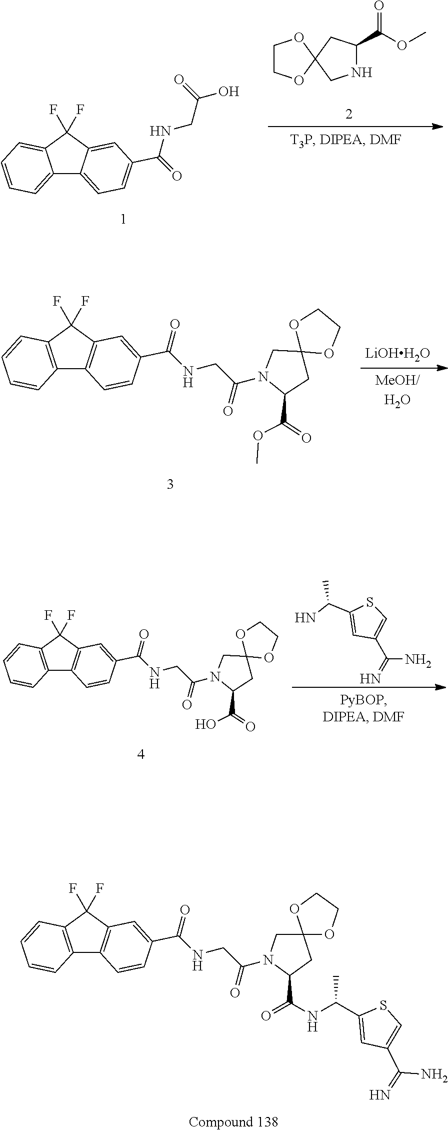

Compound 138 was prepared as a white solid from [(9,9-difluorofluoren-2-yl)formamido]acetic acid, methyl (S)-1,4-dioxa-7-azaspiro[4.4]nonane-8-carboxylate, and (R)-5-(1-aminoethyl)thiophene-3-carboximidamide based on the procedures set forth in Scheme 7. ¹H NMR (400 MHz, CD₃OD) δ 8.19 (t, J=3.1 Hz, 1H), 8.11 (s, 1H), 8.05 (d, J=8.1 Hz, 1H), 7.84-7.78 (m, 2H), 7.66 (d, J=7.7 Hz, 1H), 7.58 (t, J=7.5 Hz, 1H), 7.52 (d, J=5.7 Hz, 1H), 7.47 (t, J=7.5 Hz, 1H), 5.30 (dd, J=14.4, 7.4 Hz, 1H), 4.19 (d, J=2.9 Hz, 2H), 4.01-3.97 (m, 3H), 3.78 (t, J=7.5 Hz, 2H), 2.43 (dd, J=13.2, 8.9 Hz, 1H), 2.26-2.15 (m, 2H), 1.58 (d, J=7.0 Hz, 3H). LCMS (ESI) m/z=610 (M+H)⁺.

### Scheme 138: Synthesis of (2S,4R)—N—((R)-1-(4-carbamimidoylthiophen-2-yl)ethyl)-1-((9,9-dimethyl-9H-fluorene-3-carbonyl)glycyl)-4-fluoro-4-(methoxymethyl) pyrrolidine-2-carboxamide (Compound 139)

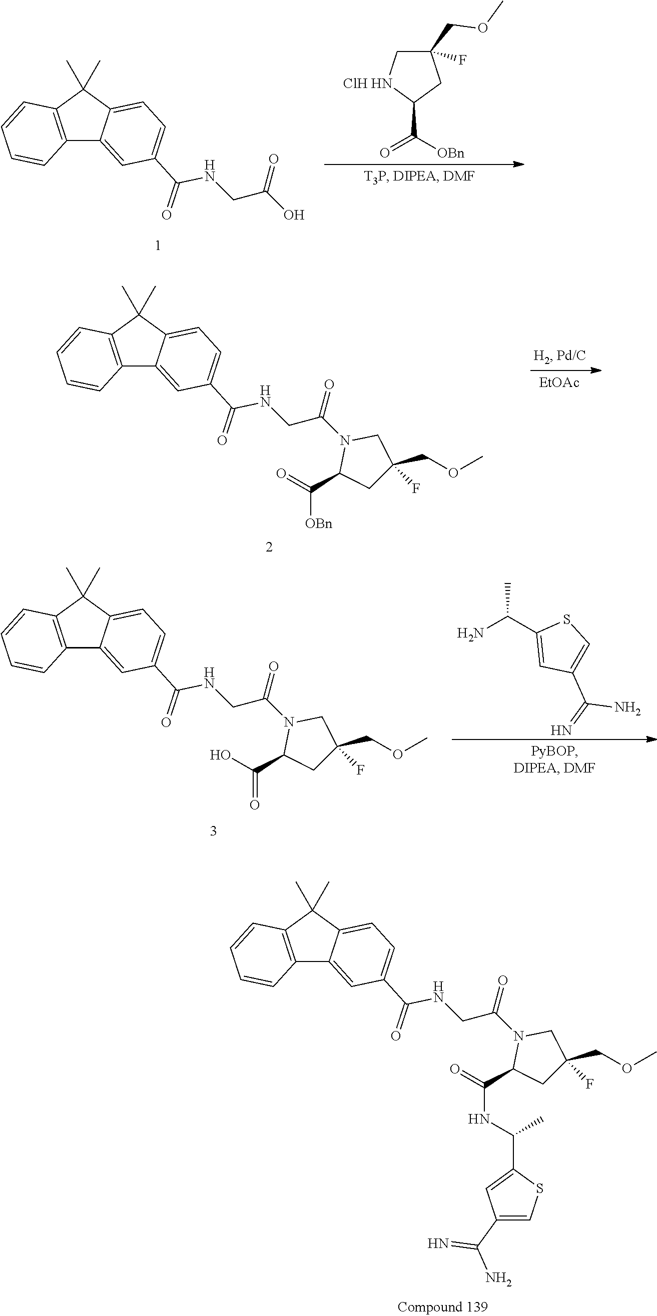

Compound 139 was prepared as a white solid from (9,9-dimethyl-9H-fluorene-3-carbonyl)glycine, benzyl (2S,4R)-4-fluoro-4-(methoxymethyl)pyrrolidine-2-carboxylate hydrochloride, and (R)-5-(1-aminoethyl)thiophene-3-carboximidamide based on the procedures set forth in Scheme 12. ¹H NMR (400 MHz, CD₃OD) δ 8.32-8.16 (m, 2H), 7.86-7.77 (m, 2H), 7.61-7.54 (m, 2H), 7.53-7.47 (m, 1H), 7.39-7.30 (m, 2H), 5.42-5.24 (m, 1H), 4.77-4.60 (m, 1H), 4.38-4.02 (m, 3H), 4.01-3.85 (m, 1H), 3.78-3.66 (m, 2H), 3.43 (d, J=4.4 Hz, 3H), 2.82-2.49 (m, 1H), 2.41-2.12 (m, 1H), 1.69-1.57 (m, 3H), 1.49 (s, 6H). LC/MS (ESI) m/z: 606(M+H)⁺.

### Scheme 139: Synthesis of (S)—N-((4-carbamimidoylthiophen-2-yl)methyl)-7-((4-(1-methyl-1H-indazol-5-yl)benzoyl)glycyl)-1,4-dioxa-7-azaspiro[4.4]nonane-8-carboxamide (Compound 140)

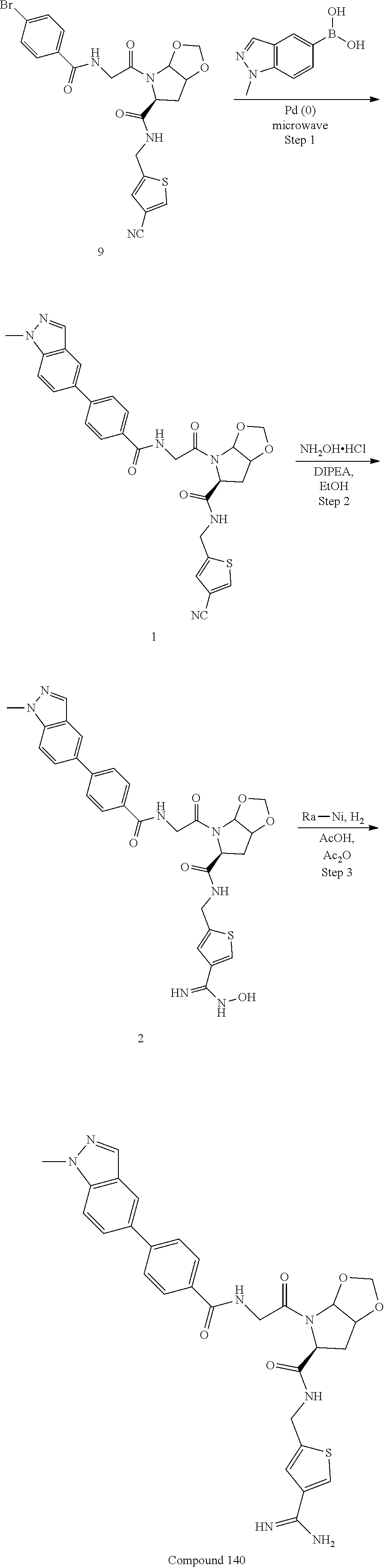

Compound 140 was prepared as a white solid from 1-methylindazol-5-yl)boronic acid, (8S)-7-[2-[(4-bromobenzoyl)amino]acetyl]-N-[(4-cyano-2-thienyl)methyl]-1,4-dioxa-7-azaspiro[4.4]nonane-8-carboxamide, and NH₂OH·HCl based on the procedures set forth in Scheme 103. LC/MS (ESI) m/z: 602 (M+H)⁺. ¹H NMR (400 MHz, DMSO-d₆) δ 9.01 (s, 1H), 8.77 (t, J=5.7 Hz, 1H), 8.61 (t, J=5.8 Hz, 1H), 8.35 (d, J=13.5 Hz, 1H), 7.97 (d, J=8.0 Hz, 2H), 7.68 (d, J=7.8 Hz, 2H), 7.56-7.40 (m, 2H), 7.31 (tt, J=8.3, 3.5 Hz, 1H), 5.15 (s, 2H), 4.70-4.56 (m, 2H), 4.50-4.40 (m, 3H), 4.43-4.33 (m, 3H), 4.31 (dd, J=16.6, 5.6 Hz, 2H), 4.02 (td, J=16.2, 5.6 Hz, 1H), 3.90 (s, 3H), 3.43 (dd, J=6.1, 2.4 Hz, 1H), 2.28 (t, J=12.3 Hz, 1H), 2.00 (dd, J=13.2, 3.1 Hz, 1H).

### Scheme 140: (S)—N-((4-carbamimidoylthiophen-2-yl)methyl)-7-((2-fluoro-[1,1′-biphenyl]-4-carbonyl)glycyl)-1,4-dioxa-7-azaspiro[4.4]nonane-8-carboxamide (Compound 141)

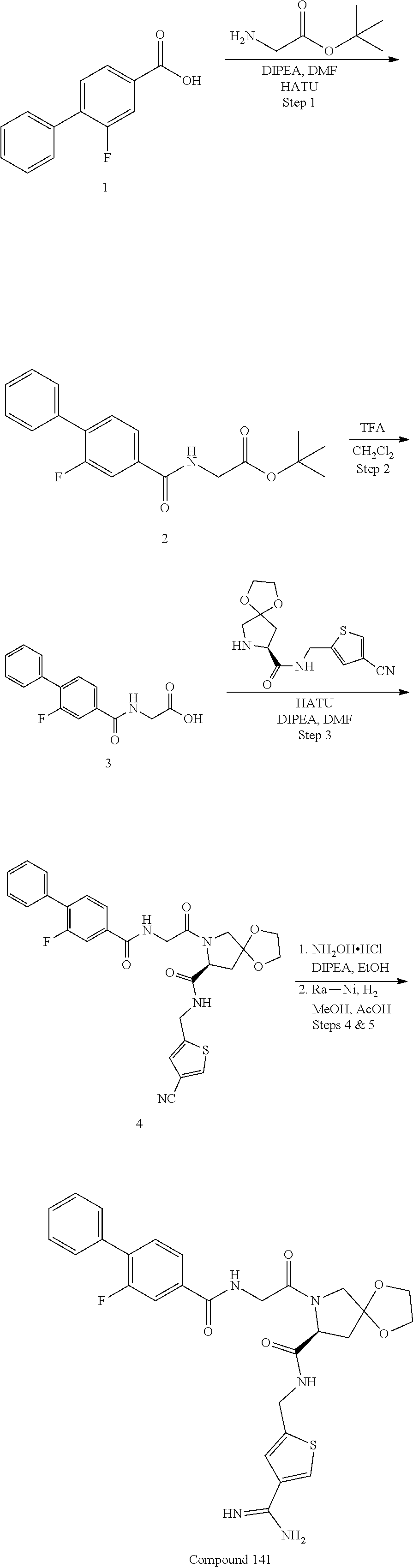

Compound 114 was prepared as a white solid from 3-fluoro-4-phenyl-benzoic acid, tert-butyl 2-aminoacetate hydrochloride, (8S)—N-[(4-cyano-2-thienyl)methyl]-1,4-dioxa-7-azaspiro[4.4]nonane-8-carboxamide, and hydroxylamine hydrochloride based on the procedures set forth in Scheme 114. LC/MS (ESI) m/z: 566 (M+H)⁺. ¹H NMR (400 MHz, DMSO) δ 8.89 (t, J=5.8 Hz, 1H), 8.64 (t, J=5.9 Hz, 1H), 8.46 (s, 2H), 8.34 (dd, J=13.7, 1.4 Hz, 1H), 7.85-7.74 (m, 2H), 7.73-7.41 (m, 8H), 4.55-4.36 (m, 3H), 4.25-4.07 (m, 1H), 4.05-3.90 (m, 3H), 3.94-3.83 (m, 2H), 3.83-3.59 (m, 2H), 2.32 (dd, J=13.1, 8.8 Hz, 1H), 2.05 (dd, J=13.1, 6.8 Hz, 1H).

### Scheme 141: Synthesis of (S)—N-((4-carbamimidoylthiophen-2-yl)methyl)-7-((6-phenylpicolinoyl)glycyl)-1,4-dioxa-7-azaspiro[4.4]nonane-8-carboxamide (Compound 145)

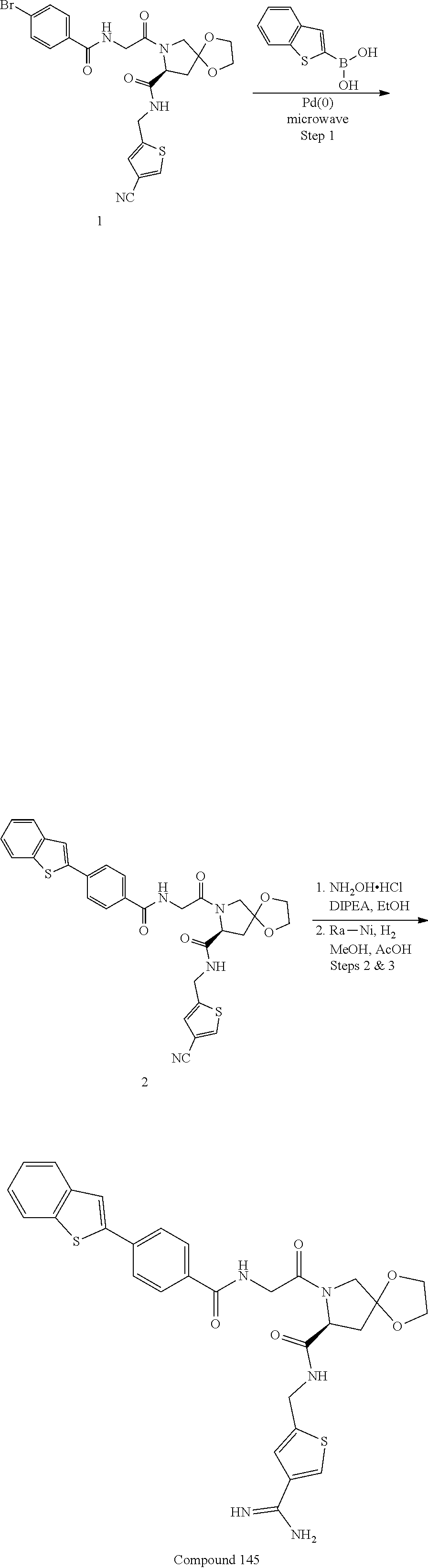

Compound 145 was prepared as a white solid from benzothiophen-6-ylboronic acid, (8S)-7-[2-[4-bromobenzoyl)amino]acetyl]-N-[(4-cyano-2-thienyl)methyl]-1,4-dioxa-7-azaspiro[4.4]nonane-8-carboxamide, and hydroxylamine hydrochloride based on the procedures set forth in Scheme 103. LC/MS (ESI) m/z: 604 (M+H)⁺. ¹H NMR (400 MHz, DMSO) δ 9.01 (s, 1H), 8.78 (s, 2H), 8.61 (s, 3H), 8.30 (d, J=12.4 Hz, 4H), 8.17-7.73 (m, 3H), 7.68-7.28 (m, 5H), 4.72 (s, 1H), 4.64-4.44 (m, 3H), 4.28-4.05 (m, 3H), 4.08-3.85 (m, 4H), 3.89-3.70 (m, 3H), 3.63 (d, J=10.9 Hz, 3H), 2.44-2.26 (m, 2H), 2.29-2.17 (m, 1H), 2.05 (dd, J=13.0, 6.8 Hz, 2H).

### Scheme 142: (S)—N-((4-carbamimidoylthiophen-2-yl)methyl)-7-((6-phenylpicolinoyl)glycyl)-1,4-dioxa-7-azaspiro[4.4]nonane-8-carboxamide (Compound 146)

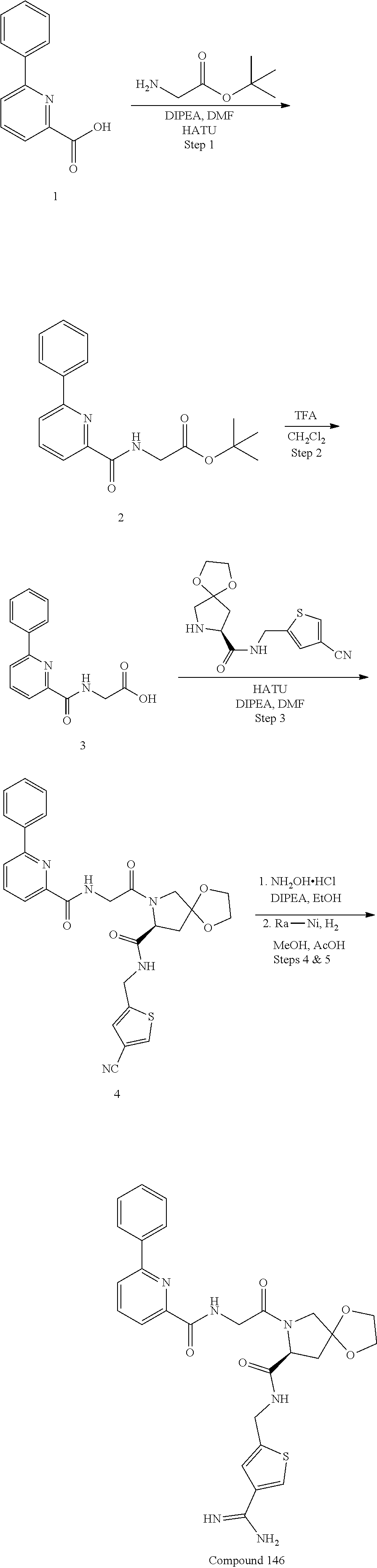

Compound 146 was prepared from 6-phenylpyridine-2-carboxylic acid, tert-butyl 2-aminoacetate hydrochloride, (8S)—N-[(4-cyano-2-thienyl)methyl]-1,4-dioxa-7-azaspiro[4.4]nonane-8-carboxamide, and hydroxylamine hydrochloride based on the procedures set forth in Scheme 114. LC/MS (ESI) m/z: 549 (M+H)⁺. ¹H NMR (400 MHz, DMSO) δ 8.99 (t, J=5.2 Hz, 1H), 8.63 (t, J=5.9 Hz, 1H), 8.34 (d, J=16.2 Hz, 1H), 8.23 (dd, J=15.3, 7.4 Hz, 3H), 8.09 (t, J=7.7 Hz, 1H), 8.00 (d, J=7.5 Hz, 1H), 7.70-7.21 (m, 3H), 4.72 (dd, J=8.9, 5.2 Hz, OH), 4.53 (d, J=5.6 Hz, OH), 4.20 (qd, J=17.2, 5.3 Hz, 2H), 4.08-3.86 (m, 4H), 3.84-3.68 (m, 1H), 3.70-3.56 (m, 1H), 2.34 (dd, J=13.1, 8.9 Hz, 1H), 2.24 (dd, J=13.2, 5.0 Hz, OH), 2.08 (dd, J=12.9, 6.5 Hz, 1H).

### Scheme 143: Synthesis of methyl ((E)-amino(5-((R)-1-((S)-7-((9,9-difluoro-9H-fluorene-3-carbonyl)glycyl)-1,4-dioxa-7-azaspiro[4.4]nonane-8-carboxamido)ethyl)thiophen-3-yl)methylene)carbamate (Compound 148)

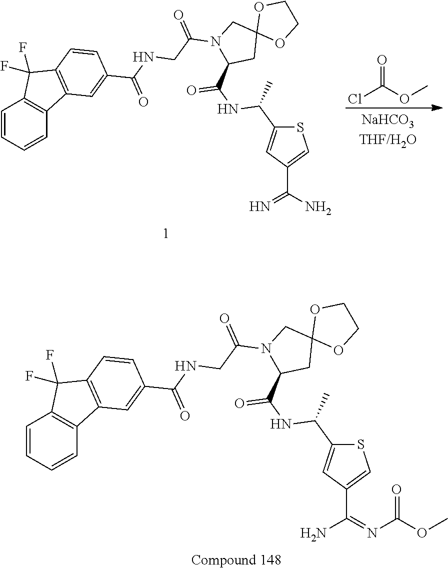

To a mixture of (S)—N—((R)-1-(4-carbamimidoylthiophen-2-yl)ethyl)-7-((9,9-difluoro-9H-fluorene-3-carbonyl)glycyl)-1,4-dioxa-7-azaspiro[4.4]nonane-8-carboxamide (140 mg, 0.23 mmol) and NaHCO₃ (58 mg, 0.69 mmol) in THF (1.5 mL) and water (0.5 mL) was added methyl carbonochloridate (32 mg, 0.35 mmol) and the mixture was stirred at room temperature for 10 minutes. The mixture was concentrated to dryness under reduced pressure. The residue was purified by prep-TLC (DCM:MeOH=10:1) and prep-HPLC to give Compound 148 (23.7 mg, yield 15.2%) as a white solid. ¹H NMR (400 MHz, CD₃OD) δ 8.17 (d, J=5.6 Hz, 1H), 8.08 (dd, J=6.9, 1.3 Hz, 1H), 7.88 (dd, J=7.8, 1.4 Hz, 1H), 7.74 (dd, J=13.7, 7.7 Hz, 2H), 7.68-7.63 (m, 2H), 7.57 (t, J=8.0 Hz, 1H), 7.44 (t, J=7.6 Hz, 1H), 5.25 (q, J=6.9 Hz, 1H), 4.58 (t, J=7.8 Hz, 1H), 4.28 (d, J=16.7 Hz, 1H), 4.16 (d, J=16.8 Hz, 1H), 4.02-3.96 (m, 4H), 3.78 (d, J=7.2 Hz, 2H), 3.58 (s, 3H), 2.43 (dd, J=13.2, 8.9 Hz, 1H), 2.22 (dd, J=13.2, 6.9 Hz, 1H), 1.58 (t, J=19.1 Hz, 3H). LC/MS (ESI) m/z: 668 (M+H)⁺.

### Scheme 144: Synthesis of phenyl ((E)-amino(5-((R)-1-((S)-7-((9,9-difluoro-9H-fluorene-3-carbonyl)glycyl)-1,4-dioxa-7-azaspiro[4.4]nonane-8-carboxamido)ethyl)thiophen-3-yl)methylene)carbamate (Compound 149)

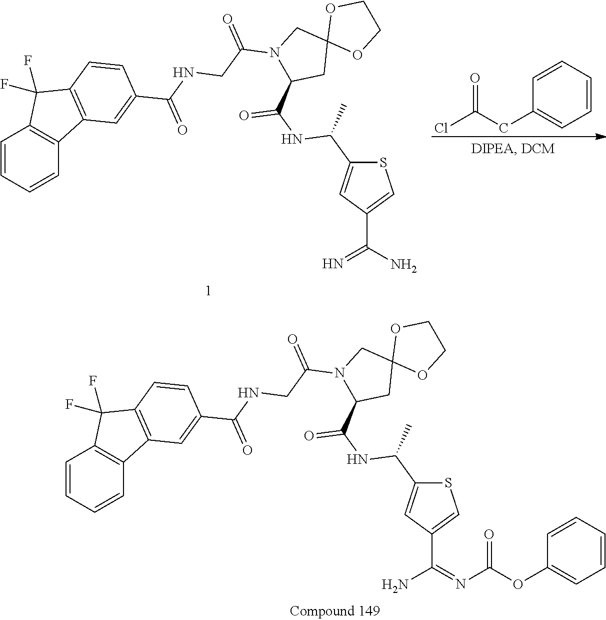 [SMILES: C[C@@H](NC(=O)[C@@H]1CC2(CN1C(=O)CNC(=O)c1ccc3c(c1)-c1ccccc1C3(F)F)OCCO2)c1cc(C(=N)N)cs1 ; O=C(Cl)Cc1ccccc1 ; C[C@@H](NC(=O)[C@@H]1CC2(CN1C(=O)CNC(=O)c1ccc3c(c1)-c1ccccc1C3(F)F)OCCO2)c1cc(/C(N)=N\C(=O)Oc2ccccc2)cs1]

To a mixture of (S)—N—((R)-1-(4-carbamimidoylthiophen-2-yl)ethyl)-7-((9,9-difluoro-9H-fluorene-3-carbonyl)glycyl)-1,4-dioxa-7-azaspiro[4.4]nonane-8-carboxamide (130 mg, 0.21 mmol) and DIPEA (81 mg, 0.63 mmol) in DCM (2 mL) was added phenyl carbonochloridate (50 mg, 0.32 mmol) and the mixture was stirred at room temperature for 10 minutes. The mixture was concentrated to dryness under reduced pressure. The residue was purified by prep-TLC (DCM:MeOH=10:1) and prep-HPLC to give Compound 149 (28.5 mg, yield 18.6%) as a white solid. ¹H NMR (400 MHz, CD₃OD) δ 8.15 (d, J=4.1 Hz, 2H), 7.86 (d, J=7.8 Hz, 1H), 7.72 (d, J=7.5 Hz, 1H), 7.68 (d, J=6.0 Hz, 2H), 7.63 (d, J=7.3 Hz, 1H), 7.53 (t, J=7.5 Hz, 1H), 7.42 (t, J=7.6 Hz, 1H), 7.35 (t, J=7.9 Hz, 2H), 7.20 (t, J=7.4 Hz, 1H), 7.09 (t, J=7.4 Hz, 2H), 5.28 (q, J=6.7 Hz, 1H), 4.60 (dd, J=8.6, 6.7 Hz, 1H), 4.25 (d, J=16.6 Hz, 1H), 4.17 (d, J=16.7 Hz, 1H), 4.03-3.96 (m, 4H), 3.81-3.74 (m, 2H), 2.44 (dd, J=13.1, 8.8 Hz, 1H), 2.23 (dd, J=13.1, 6.5 Hz, 1H), 1.62 (dd, J=37.4, 6.9 Hz, 3H). LC/MS (ESI) m/z: 730 (M+H)⁺.

### Scheme 145: Synthesis of 8-(((3R,5S)-5-(((R)-1-(4-carbamimidoylthiophen-2-yl)ethyl) carbamoyl)-1-((9,9-difluoro-9H-fluorene-3-carbonyl)glycyl)-3-fluoropyrrolidin-3-yl)methoxy)octanoic acid (Compound 150)

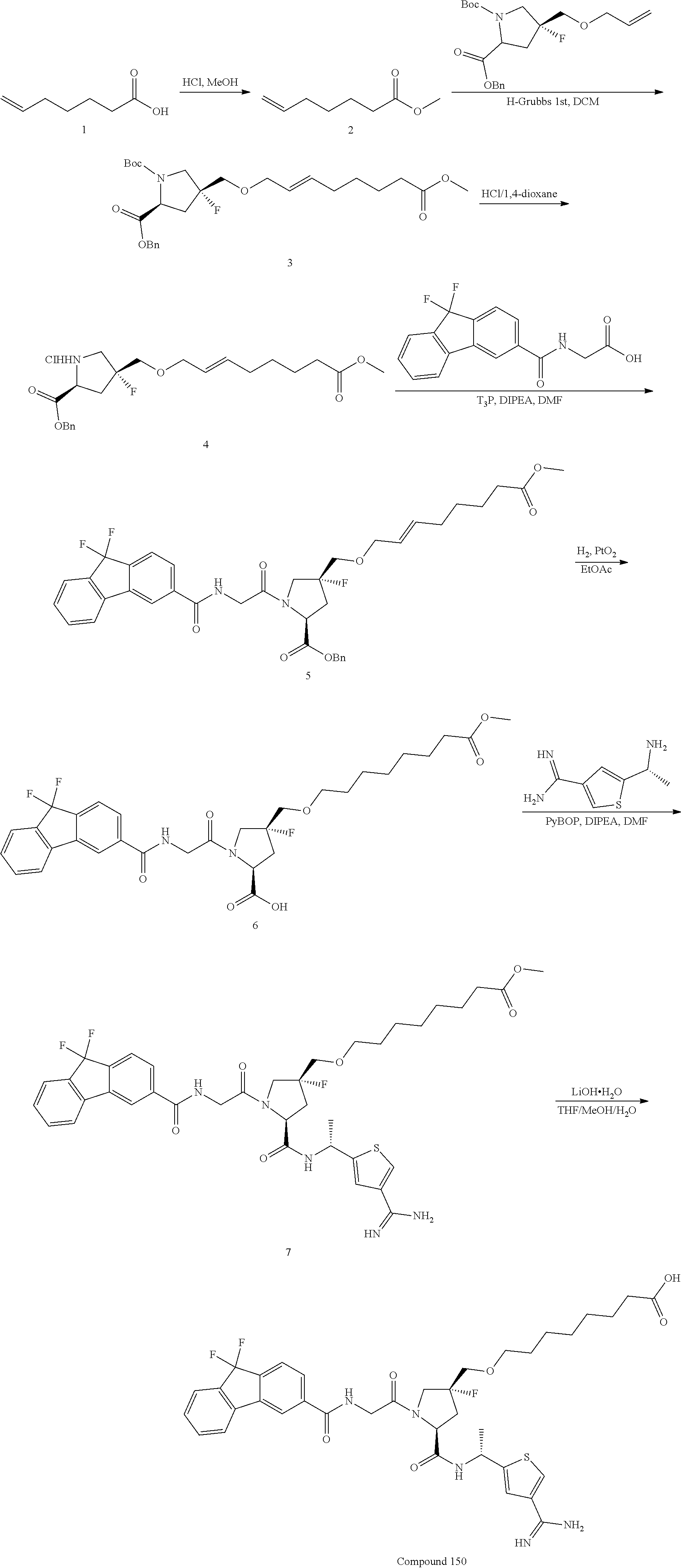

### Step 1: methyl hept-6-enoate (2)

To a solution of hept-6-enoic acid (1 g, 7.81 mmol) in MeOH (10 mL) was added conc. HCl (10 drops) at 0° C. and the mixture was stirred at 60° C. overnight. The mixture was diluted with EtOAc, washed with water and brine, dried over anhydrous Na₂SO₄, filtered, and concentrated under reduced pressure to dryness. The residue was purified by flash chromatography on silica gel (PE:EtOAc=20: 1) to give methyl hept-6-enoate (610 mg, yield 54.7%) as a colorless oil. LC/MS (ESI) m/z: 143 (M+H)⁺.

### Step 2: 2-benzyl 1-(tert-butyl) (2S,4R)-4-fluoro-4-((((E)-6-methoxy-8-oxooct-2-en-1-yl)oxy) methyl)pyrrolidine-1,2-dicarboxylate (3)

To a mixture of 2-benzyl 1-(tert-butyl) (2S,4R)-4-((allyloxy)methyl)-4-fluoropyrrolidine-1,2-dicarboxylate (134 mg, 0.34 mmol) and methyl hept-6-enoate (73 mg, 0.51 mmol) in dry DCM (2 mL) was added H-Grubbs 1^{st} (51 mg, 0.085 mmol) under N₂ atmosphere and the mixture was stirred at 25° C. for 18 hours under N₂ atmosphere. The mixture was concentrated to dryness under reduced pressure and the residue was purified by flash chromatography (PE:EtOAc=10:1) to give 2-benzyl 1-(tert-butyl) (2S,4R)-4-fluoro-4-((((E)-8-methoxy-8-oxooct-2-en-1-yl)oxy)methyl)pyrrolidine-1,2-dicarboxylate (70 mg, yield 40.7%) as a brown oil. LC/MS (ESI) m/z: 508 (M+H)⁺.

### Step 3: benzyl (2S,4R)-4-fluoro-4-((((E)-8-methoxy-8-oxooct-2-en-1-yl)oxy)methyl) pyrrolidine-2-carboxylate hydrochloride (4)

To a solution of 2-benzyl 1-(tert-butyl) (2S,4R)-4-fluoro-4-((((E)-8-methoxy-8-oxooct-2-en-1-yl)oxy)methyl)pyrrolidine-1,2-dicarboxylate (70 mg, 0.14 mmol) in DCM (1 mL) was added HCl/1,4-dioxane (1 mL) and the mixture was stirred at room temperature for 2 hours. The reaction mixture was concentrated to dryness under reduced pressure to give benzyl(2S,4R)-4-fluoro-4-((((E)-8-methoxy-8-oxooct-2-en-1-yl)oxy)methyl)pyrrolidine-2-carboxylate hydrochloride (65 mg, crude) as a white solid, which was used directly in the next step without further purification. LC/MS (ESI) m/z: 408 (M+H)⁺.

### Step 4: benzyl (2S,4R)-1-((9,9-difluoro-9H-fluorene-3-carbonyl)glycyl)-4-fluoro-4-((((E)-8-methoxy-8-oxooct-2-en-1-yl)oxy)methyl)pyrrolidine-2-carboxylate (5)

To a mixture of benzyl (2S,4R)-4-fluoro-4-((((E)-8-methoxy-8-oxooct-2-en-1-yl)oxy)methyl) pyrrolidine-2-carboxylate hydrochloride (65 mg, 0.16 mmol) and (9,9-difluoro-9H-fluorene-3-carbonyl)glycine (40 mg, 0.13 mmol) in DMF (1 mL) was added DIPEA (86 mg, 0.67 mmol) and T₃P (127 mg, 0.20 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography on silica gel (DCM:MeOH=20:1) to give benzyl (2S,4R)-1-((9,9-difluoro-9H-fluorene-3-carbonyl)glycyl)-4-fluoro-4-((((E)-8-methoxy-8-oxooct-2-en-1-yl)oxy)methyl)pyrrolidine-2-carboxylate (55 mg, yield 59.8%) as a colorless oil. LC/MS (ESI) m/z: 693 (M+H)⁺.

### Step 5: (2S,4R)-1-((9,9-difluoro-9H-fluorene-3-carbonyl)glycyl)-4-fluoro-4-(((6-methoxy-6-oxooctyl)oxy)methyl)pyrrolidine-2-carboxylic acid (6)

To a solution of benzyl (2S,4R)-1-((9,9-difluoro-9H-fluorene-3-carbonyl)glycyl)-4-fluoro-4-((((E)-8-methoxy-8-oxooct-2-en-1-yl)oxy)methyl)pyrrolidine-2-carboxylate (55 mg, 0.079 mmol) in EtOAc (2 mL) was added PtO₂ (4 mg, 0.016 mmol) at room temperature and the mixture was stirred under H₂ atmosphere at room temperature for 30 minutes. The mixture was filtered, and the filtrate was concentrated to dryness under reduced pressure to give (2S,4R)-1-((9,9-difluoro-9H-fluorene-3-carbonyl)glycyl)-4-fluoro-4-(((8-methoxy-8-oxooctyl)oxy)methyl)pyrrolidine-2-carboxylic acid (43 mg, yield 89.6%) as a colorless oil, which was used directly in the next step. LC/MS (ESI) (m/z): 605 (M+H)⁺.

### Step 6: methyl 8-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)octanoate (7)

To a mixture of (2S,4R)-1-((9,9-difluoro-9H-fluorene-3-carbonyl)glycyl)-4-fluoro-4-(((8-methoxy-8-oxooctyl)oxy)methyl)pyrrolidine-2-carboxylic acid (43 mg, 0.071 mmol) and (R)-5-(1-aminoethyl)thiophene-3-carboximidamide hydrochloride (15 mg, 0.085 mmol) in DMF (1 mL) was added DIPEA (46 mg, 0.36 mmol) and PyBOP (44 mg, 0.085 mmol) at 0° C. and the mixture was stirred under N₂ atmosphere at room temperature for 1 hour. The reaction mixture was diluted with water and extracted with EtOAc twice. The combined organic layers were washed with brine, dried over anhydrous Na₂SO₄, filtered, and concentrated to dryness under reduced pressure. The residue was purified by prep-TLC (DCM:MeOH=10:1) to give methyl 8-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)octanoate (24 mg, yield 44.4%) as a white solid. LCMS (ESI) (m/z): 756 (M+H)⁺.

### Step 7: 8-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)octanoic acid (Compound 150)

To a solution of methyl 8-(((3R,5S)-5-(((R)-1-(4-carbamimidoylthiophen-2-yl)ethyl) carbamoyl)-1-((9,9-difluoro-9H-fluorene-3-carbonyl)glycyl)-3-fluoropyrrolidin-3-yl)methoxy)octanoate (24 mg, 0.032 mmol) in MeOH (0.75 mL) and water (0.25 mL) was added LiOH·H₂O (3 mg, 0.064 mmol) at 0° C. and the mixture was stirred at room temperature for 3 hours. The mixture was acidified with 1N aq·HCl to pH-4 and concentrated to dryness under reduced pressure. The residue was purified by prep-HPLC to give Compound 150 (1.7 mg, yield 7.2%) as a white solid. ¹H NMR (400 MHz, CD₃OD) δ 8.27 (dd, J=12.4, 1.5 Hz, 1H), 8.18 (s, 1H), 7.87 (dd, J=7.8, 1.2 Hz, 1H), 7.73 (dd, J=18.4, 7.6 Hz, 2H), 7.65 (d, J=7.5 Hz, 1H), 7.57 (d, J=8.0 Hz, 1H), 7.53 (s, 1H), 7.45 (t, J=7.5 Hz, 1H), 5.28 (q, J=6.9 Hz, 1H), 4.65 (d, J=8.6 Hz, 1H), 4.60 (d, J=10.9 Hz, 4H), 4.24 (d, J=16.3 Hz, 2H), 4.04-3.89 (m, 2H), 3.70 (dd, J=15.3, 5.7 Hz, 2H), 3.52 (t, J=6.3 Hz, 2H), 2.62-2.51 (m, 1H), 2.32-2.20 (m, 1H), 2.16 (t, J=7.4 Hz, 2H), 1.59 (d, J=6.9 Hz, 7H), 1.34 (s, 2H). LCMS (ESI) (m/z): 742 (M+H)⁺.

### Scheme 146: Synthesis of (S)—N—((S)-1-(4-carbamimidoylthiophen-2-yl)ethyl)-7-((4-(5-chloropyridin-2-yl)benzoyl)glycyl)-1,4-dioxa-7-azaspiro[4.4]nonane-8-carboxamide (Compound 151)

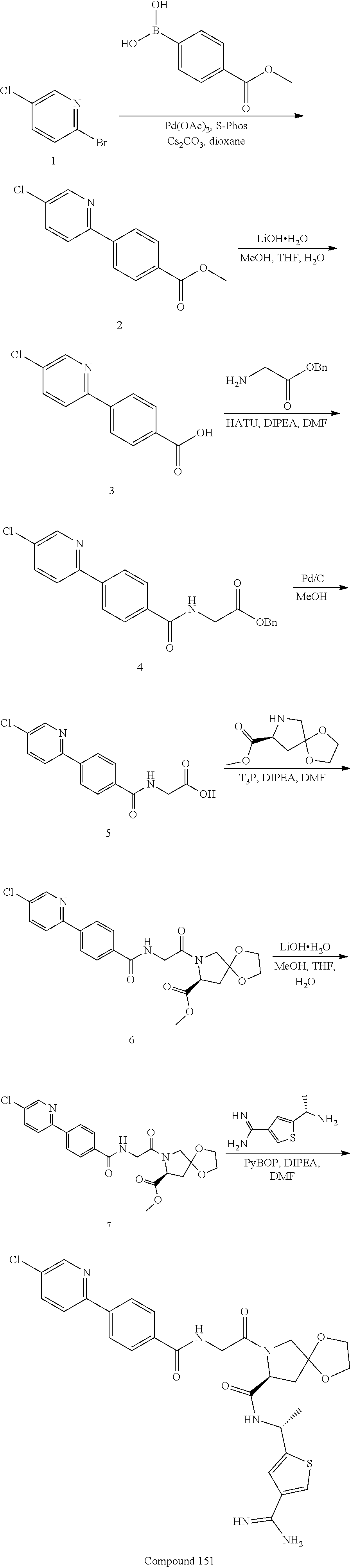

### Step 1: methyl 4-(5-chloropyridin-2-yl)benzoate (2)

To a mixture of 2-bromo-5-chloropyridine (1.0 g, 4.18 mmol) and (4-(methoxycarbonyl) phenyl)boronic acid (693 mg, 4.18 mmol) in 1,4-dioxane (15 mL) was added Cs₂CO₃ (3.4 g, 10.4 mmol), S-Phos (86 mg, 0.21 mmol) and Pd(OAc)₂ (47 g, 0.21 mmol) at 25° C. under N₂ atmosphere and the reaction mixture was stirred at 110° C. overnight. The mixture was diluted with water and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=10:1) to give methyl 4-(5-chloropyridin-2-yl)benzoate (330 mg, yield 31.9%) as a colorless oil. LC/MS (ESI) m/z:248 (M+H)⁺.

### Step 2: 4-(5-chloropyridin-2-yl)benzoic acid (3)

To a solution of methyl 4-(5-chloropyridin-2-yl)benzoate (300 mg, 1.21 mmol) in MeOH (4 mL), THF (2 ml) and water (2 mL) was added LIOH·H₂O (172 mg, 3.63 mmol) at 0° C. and the mixture was stirred at room temperature for 2 hours. The mixture was acidified with 1 N aq·HCl to pH-3, extracted with EtOAc twice. The combined organic layers were washed with brine, dried over anhydrous Na₂SO₄, filtered, and concentrated under reduced pressure to give 4-(5-chloropyridin-2-yl)benzoic acid (270 mg, yield 95.4%) as a white solid, which was used directly in the next reaction. LC/MS (ESI) (m/z): 234 (M+H)⁺.

### Step 3: benzyl (4-(5-chloropyridin-2-yl)benzoyl)glycinate (4)

To a mixture of 4-(5-chloropyridin-2-yl)benzoic acid (270 mg, 1.16 mmol) and benzyl glycinate (229 mg, 1.39 mmol) in DMF (6 ml-) was added HBTU (657 mg, 1.73 mmol) and DIPEA (747 mg, 5.78 mmol) under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography on silica gel (PE:EtOAc=3:1) to give benzyl (4-(5-chloropyridin-2-yl)benzoyl)glycinate (248 mg, yield 56.4%) as a colorless oil. LC/MS (ESI) m/z: 381 (M+H)⁺.

### Step 4: (4-(5-chloropyridin-2-yl)benzoyl)glycine (5)

To a solution of benzyl (4-(5-chloropyridin-2-yl)benzoyl)glycinate (240 mg, 0.63 mmol) in MeOH (4 mL)was added Pd/C, 10% wt. (66 mg, 0.063 mmol). The flask was evacuated and backfilled with H₂ gas in a balloon. The reaction was stirred at room temperature overnight. The reaction mixture was filtered through Celited pad. The filtrate was concentracted to give a white solid., which was used directly in the next reaction. LC/MS (ESI) (m/z): 291 (M+H)⁺.

### Step 5: methyl (S)-7-((4-(5-chloropyridin-2-yl)benzoyl)glycyl)-1,4-dioxa-7-azaspiro[4.4]nonane-8-carboxylate (6)

To a mixture of (4-(5-chloropyridin-2-yl)benzoyl)glycine (93 mg, 0.32 mmol) and methyl (S)-1,4-dioxa-7-azaspiro[4.4]nonane-8-carboxylate (60 mg, 0.32 mmol) in DMF (2 mL) was added DIPEA (207 mg, 1.60 mmol) and T₃P (306 mg, 0.48 mmol, 50% wt. in EtOAc) under N₂ atmosphere and the mixture was stirred at room temperature for 1 hours. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography on silica gel (DCM:MeOH=20:1) to give methyl (S)-7-((4-(5-chloropyridin-2-yl)benzoyl)glycyl)-1,4-dioxa-7-azaspiro[4.4]nonane-8-carboxylate (40 mg, yield 27.1%) as a colorless oil. LC/MS (ESI) m/z: 460 (M+H)⁺.

### Step 6: (S)-7-((4-(5-chloropyridin-2-yl)benzoyl)glycyl)-1,4-dioxa-7-azaspiro[4.4]nonane-8-carboxylic acid (7)

To a solution of methyl (S)-7-((4-(5-chloropyridin-2-yl)benzoyl)glycyl)-1,4-dioxa-7-azaspiro [4.4]nonane-8-carboxylate (40 mg, 0.087 mmol) in MeOH (2 mL), THF (1 mL), and water (1 mL) was added LiOH·H₂O (11 mg, 0.26 mmol) at 0° C. and the mixture was stirred at room temperature for 1 hours. The mixture was acidified with 1 N aq·HCl to pH˜4, extracted with EtOAc twice. The combined organic layers were washed with brine, dried over anhydrous Na₂SO₄, filtered, and concentrated under reduced pressure to give (S)-7-((4-(5-chloropyridin-2-yl)benzoyl) glycyl)-1,4-dioxa-7-azaspiro[4.4]nonane-8-carboxylic acid (35 mg, yield 90.3%) as a white solid which was used directly in the next reaction. LC/MS (ESI) (m/z): 446 (M+H)⁺.

### Step 7: (S)—N—((S)-1-(4-carbamimidoylthiophen-2-yl)ethyl)-7-((4-(5-chloropyridin-2-yl) benzoyl)glycyl)-1,4-dioxa-7-azaspiro[4.4]nonane-6-carboxamide (Compound 151)

To a mixture of (S)-7-((4-(5-chloropyridin-2-yl)benzoyl)glycyl)-1,4-dioxa-7-azaspiro[4.4]nonane-8-carboxylic acid (35 mg, 0.078 mmol) and (S)-5-(1-aminoethyl)thiophene-3-carboximidamide (16 mg, 0.094 mmol) in DMF (2 mL) was added DIPEA (51 mg, 0.39 mmol) and PyBOP (61 mg, 0.12 mmol) at 0° C. under N₂ atmosphere and the mixture was stirred at 25° C. for 1 hour. The reaction mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (DCM:MeOH=10:1) and prep-HPLC to give Compound 151 (2.0 mg, yield 4.3%) as a white solid. ¹H NMR (400 MHz, CD₃OD) δ 8.66 (s, 1H), 8.53 (s, 1H), 8.33-8.21 (m, 1H), 8.12 (d, J=8.5 Hz, 2H), 7.99-7.94 (m, 4H), 7.59-7.51 (m, 1H), 5.31 (dq, J=13.8, 6.8 Hz, 1H), 4.56 (s, 1H), 4.19 (d, J=2.9 Hz, 2H), 3.99 (dt, J=12.3, 5.5 Hz, 4H), 3.86-3.74 (m, 2H), 2.48-2.33 (m, 1H), 2.21 (dd, J=13.2, 6.6 Hz, 1H), 1.68-1.57 (m, 3H). LC/MS (ESI) m/z: 597 (M+H)⁺.

### Scheme 147: Synthesis of (S)—N—((R)-1-(4-(2H-1,2,3-triazol-4-yl)thiophen-2-yl)ethyl)-7-((9,9-difluoro-9H-fluorene-3-carbonyl)glycyl)-1,4-dioxa-7-azaspiro[4.4]nonane-8-carboxamide (Compound 152)

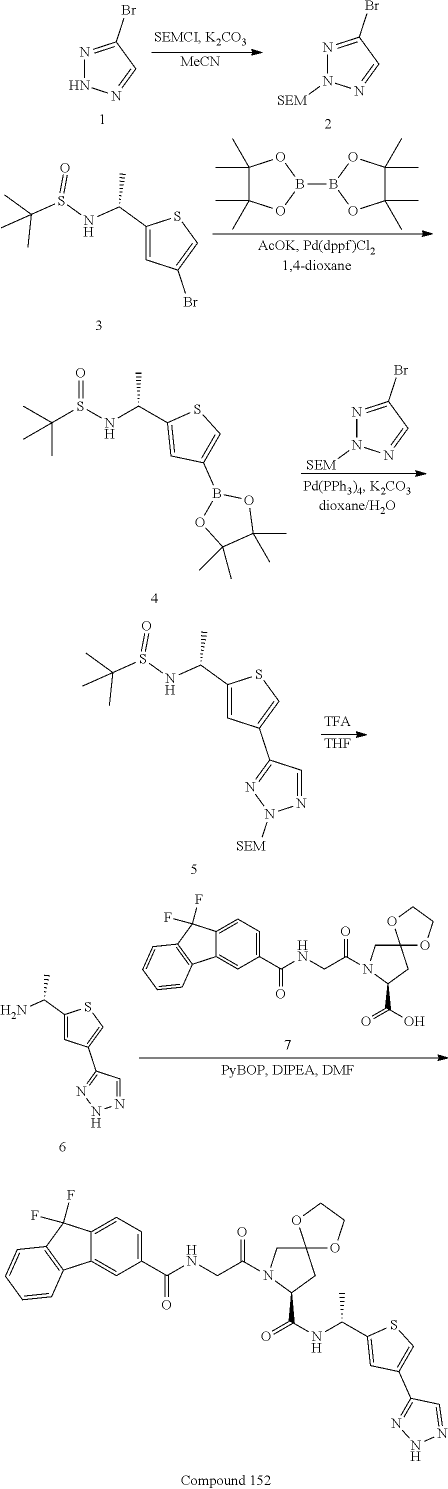

### Step 1: 4-bromo-2-((2-(trimethylsilyl)ethoxy)methyl)-2H-1,2,3-triazole (2)

To a solution of 4-bromo-2H-1,2,3-triazole (200 mg, 1.4 mmol) in MeCN (3 mL) was added K₂CO₃ (773 mg, 5.6 mmol) and SEMCl (467 mg, 2.8 mmol) under N₂ atmosphere at room temperature and stirred overnight. The mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (PE:EtOAc=2:1) to give 4-bromo-2-((2-(trimethylsilyl)ethoxy)methyl)-2H-1,2,3-triazole (50 mg, yield 13.2%) as a yellow oil. LCMS (ESI) m/z=278 (M+H)⁺.

### Step 2: 2-methyl-N—((R)-1-(4-(4,4,5,5-tetramethyl-1,3,2-dioxaborolan-2-yl)thiophen-2-yl) ethyl)propane-2-sulfinamide (4)

To a solution of N—((R)-1-(4-bromothiophen-2-yl)ethyl)-2-methylpropane-2-sulfinamide (62 mg, 0.2 mmol) in 1,4-dioxane (2 mL) was added Pin₂B₂ (100 mg, 0.40 mmol), AcOK (78 mg, 0.8 mmol) and Pd(dppf)Cl₂ (15 mg, 0.02 mmol). The mixture was degassed under N₂ atmosphere and stirred at 85° C. for 2 hours. The mixture was used directly in next step without purification.

### Step 3: 2-methyl-N—((R)-1-(4-(2-((2-(trimethylsilyl)ethoxy)methyl)-2H-1,2,3-triazol-4-yl) thiophen-2-yl)ethyl)propane-2-sulfinamide (5)

To the mixture of previous step was added 4-bromo-2-((2-(trimethylsilyl) ethoxy)methyl)-2H-1,2,3-triazole (64 mg, 0.18 mmol), Pd(PPh₃)₄ (21 mg, 0.018 mmol), K₂CO₃ (100 mg, 0.72 mmol), 1,4-dioxane (0.5 mL) and water (1.5 mL) and was degassed under N₂ atmosphere and stirred at 85° C. for 2 hours. The mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (PE:EtOAc=1:1) to give 2-methyl-N—((R)-1-(4-(2-((2-(trimethylsilyl)ethoxy)methyl)-2H-1,2,3-triazol-4-yl)thiophen-2-yl)ethyl)propane-2-sulfinamide (53 mg, two steps yield 60%) as a colorless oil. LCMS (ESI) m/z=429 (M+H)⁺.

### Step 4: (R)-1-(4-(2H-1,2,3-triazol-4-yl)thiophen-2-yl)ethan-1-amine (6)

A solution of 2-methyl-N—((R)-1-(4-(2-((2-(trimethylsilyl)ethoxy)methyl)-2H-1,2,3-triazol-4-yl)thiophen-2-yl)ethyl)propane-2-sulfinamide (53 mg, 0.12 mmol) in TFA (1 mL) was stirred at room temperature overnight. The mixture was diluted with water and extracted with EtOAc twice. The combined organic layers were washed with brine, dried over anhydrous Na₂SO₄, filtered, and concentrated to dryness under reduced pressure to give (R)-1-(4-(2H-1,2,3-triazol-4-yl)thiophen-2-yl)ethan-1-amine (35 mg crude), which was used directly in next step without purification.

### Step 5: (S)—N—((R)-1-(4-(2H-1,2,3-triazol-4-yl)thiophen-2-yl)ethyl)-7-((9,9-difluoro-9H-fluorene-3-carbonyl)glycyl)-1,4-dioxa-7-azaspiro[4.4]nonane-8-carboxamide (Compound 152)

To a solution of (R)-1-(4-(2H-1,2,3-triazol-4-yl)thiophen-2-yl)ethan-1-amine (35 mg crude) in DMF (1 mL) was added (S)-7-((9,9-difluoro-9H-fluorene-3-carbonyl)glycyl)-1,4-dioxa-7-azaspiro[4.4]nonane-8-carboxylic acid (46 mg, 0.10 mmol), DIPEA (77 mg, 0.6 mmol) and PyBOP (62 mg, 0.12 mmol) at 0° C. and the mixture was stirred under N₂ atmosphere at room temperature for 2 hours. The mixture was diluted with water and extracted with EtOAc twice. The combined organic layers were washed with brine, dried over anhydrous Na₂SO₄, filtered, and concentrated to dryness under reduced pressure. The residue was purified by flash chromatography on silica gel (DCM:MeOH=9:1) and prep-HPLC to give Compound 152 (3 mg, yield 4.7%) as a white solid. ¹H NMR (400 MHz, CD₃OD) δ 8.15 (d, J=9.1 Hz, 1H), 7.85 (dt, J=9.8, 4.8 Hz, 1H), 7.73-7.60 (m, 4H), 7.57-7.47 (m, 2H), 7.43 (t, J=7.4 Hz, 1H), 5.32 (q, J=6.6 Hz, 1H), 4.63-4.57 (m, 3H), 4.20 (q, J=16.7 Hz, 2H), 4.00 (s, 3H), 3.78 (d, J=19.1 Hz, 2H), 2.47-2.37 (m, 1H), 2.26-2.18 (m, 1H), 1.60 (t, J=16.9 Hz, 3H). LC/MS (ESI) (m/z): 635 (M+H)⁺.

### Scheme 148: Synthesis of (2S,4R)—N—((R)-1-(4-carbamimidoylthiophen-2-yl)ethyl)-1-((9,9-dimethyl-9H-fluorene-3-carbonyl)glycyl)-4-fluoro-4-(fluoromethyl)pyrrolidine-2-carboxamide (Compound 153)

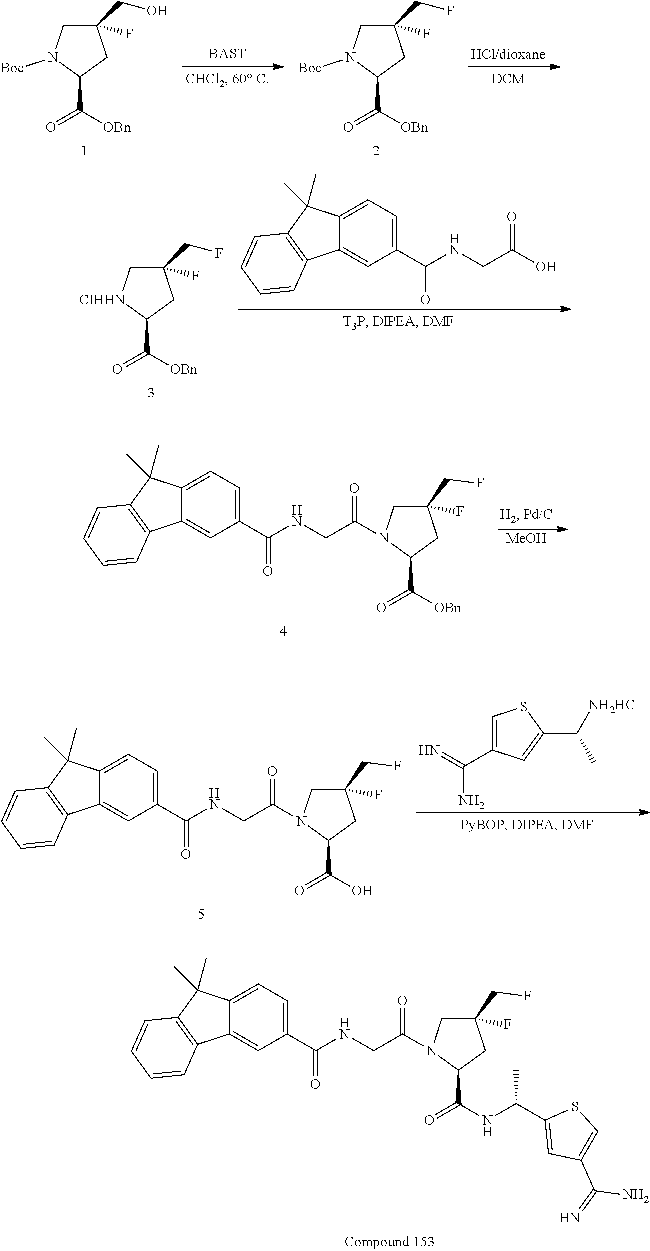

### Step 1: 2-benzyl 1-(tert-butyl) (2S,4R)-4-fluoro-4-(fluoromethyl) pyrrolidine-1,2-dicarboxylate (2)

To a solution of 2-benzyl 1-(tert-butyl) (2S,4R)-4-fluoro-4-(hydroxymethyl) pyrrolidine-1,2-dicarboxylate (4.1 g, 11.67 mmol) in CHCl₃ (40 mL) was added BAST (10.3 g, 46.68 mmol) at 0° C. and the mixture was stirred at 60° C. overnight. The mixture was diluted with CHCl₃, washed with water and brine, dried over anhydrous Na₂SO₄, filtered, and concentrated under reduced pressure to dryness. The residue was purified by flash chromatography on silica gel (PE:EtOAc=5:1) to give 2-benzyl 1-(tert-butyl) (2S,4R)-4-fluoro-4-(fluoromethyl)pyrrolidine-1,2-dicarboxylate (3.53 g, yield 85.1%) as a colorless oil. LC/MS (ESI) m/z: 356 (M+H)⁺.

### Step 2: benzyl (2S,4R)-4-fluoro-4-(fluoromethyl)pyrrolidine-2-carboxylate hydrochloride (3)

To a solution of 2-benzyl 1-(tert-butyl) (2S,4R)-4-fluoro-4-(fluoromethyl)pyrrolidine-1,2-dicarboxylate (300 mg, 0.84 mmol) in DCM (3 mL) was added HCl/1,4-dioxane (2 mL) and the mixture was stirred at room temperature for 2 hours. The reaction mixture was concentrated to dryness under reduced pressure to give benzyl (2S,4R)-4-fluoro-4-(fluoromethyl)pyrrolidine-2-carboxylate hydrochloride (300 mg, crude) as a white solid, which was used directly in the next step without further purification. LC/MS (ESI) m/z: 256 (M+H)⁺.

### Step 3: benzyl (2S,4R)-1-((9,9-dimethyl-9H-fluorene-3-carbonyl)glycyl)-4-fluoro-4-(fluoromethyl)pyrrolidine-2-carboxylate (4)

To a mixture of benzyl (2S,4R)-4-fluoro-4-(fluoromethyl)pyrrolidine-2-carboxylate hydrochloride (59 mg, 0.23 mmol) and (9,9-dimethyl-9H-fluorene-3-carbonyl)glycine (45 mg, 0.15 mmol) in DMF (1.0 mL) was added DIPEA (98 mg, 0.76 mmol) and T₃P (146 mg, 0.23 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography on silica gel (PE:EtOAc=1:3) to give benzyl (2S,4R)-1-((9,9-dimethyl-9H-fluorene-3-carbonyl)glycyl)-4-fluoro-4-(fluoromethyl)pyrrolidine-2-carboxylate (36 mg, yield 44.4%) as a colorless oil. LC/MS (ESI) m/z: 533 (M+H)⁺.

### Step 4: (2S,4R)-1-((9,9-dimethyl-9H-fluorene-3-carbonyl)glycyl)-4-fluoro-4-(fluoromethyl) pyrrolidine-2-carboxylic acid (5)

To a solution of benzyl (2S,4R)-1-((9,9-dimethyl-9H-fluorene-3-carbonyl)glycyl)-4-fluoro-4-(fluoromethyl)pyrrolidine-2-carboxylate (36 mg, 0.068 mmol) in MeOH (3 mL) was added Pd/C (4 mg, 10% wt.) at room temperature and the mixture was stirred under H₂ atmosphere at room temperature for 2 hours. The mixture was filtered, and the filtrate was concentrated to dryness under reduced pressure to give (2S,4R)-1-((9,9-dimethyl-9H-fluorene-3-carbonyl)glycyl)-4-fluoro-4-(fluoromethyl)pyrrolidine-2-carboxylic acid (27 mg, yield 90.0%) as a colorless oil, which was used directly in the next step. LC/MS (ESI) (m/z): 443 (M+H)⁺.

### Step 5: (2S,4R)—N—((R)-1-(4-carbamimidoylthiophen-2-yl)ethyl)-1-((9,9-dimethyl-9H-fluorene-3-carbonyl)glycyl)-4-fluoro-4-(fluoromethyl)pyrrolidine-2-carboxamide (Compound 153)

To a mixture of (2S,4R)-1-((9,9-dimethyl-9H-fluorene-3-carbonyl)glycyl)-4-fluoro-4-(fluoromethyl)pyrrolidine-2-carboxylic acid (27 mg, 0.06 mmol) and (R)-5-(1-aminoethyl)thiophene-3-carboximidamide hydrochloride (15 mg, 0.073 mmol) in DMF (1 mL) was added DIPEA (39 mg, 0.30 mmol) and PyBOP (34 mg, 0.066 mmol) at 0° C. and the mixture was stirred under N₂ atmosphere at room temperature for 1 hour. The reaction mixture was diluted with water and extracted with EtOAc twice. The combined organic layers were washed with brine, dried over anhydrous Na₂SO₄, filtered, and concentrated to dryness under reduced pressure. The residue was purified by prep-TLC (DCM:MeOH=10:1) and prep-HPLC to give Compound 153 (1.6 mg, yield 4.5%) as a white solid. ¹H NMR (400 MHz, CD₃OD) δ 8.26 (dd, J=8.1, 1.1 Hz, 1H), 8.08 (d, J=1.3 Hz, 1H), 7.84-7.79 (m, 2H), 7.58 (d, J=7.9 Hz, 1H), 7.53-7.49 (m, 2H), 7.37-7.35 (m, 2H), 5.28 (q, J=6.8 Hz, 1H), 4.81-4.75 (m, 1H), 4.67 (dd, J=17.8, 8.3 Hz, 2H), 4.35 (d, J=16.7 Hz, 1H), 4.20 (s, 1H), 4.14 (d, J=14.8 Hz, 1H), 3.97 (dd, J=32.0, 12.3 Hz, 1H), 2.69-2.56 (m, 1H), 2.30-2.17 (m, 1H), 1.61 (t, J=14.7 Hz, 3H), 1.50 (s, 6H). LCMS (ESI) (m/z): 594 (M+H)⁺.

### Scheme 149: Synthesis of (2S,4R)—N—((R)-1-(4-carbamimidoylthiophen-2-yl)ethyl)-1-((4-(difluoro(phenyl)methyl)benzoyl)glycyl)-4-fluoro-4-(fluoromethyl)pyrrolidine-2-carboxamide (Compound 154)

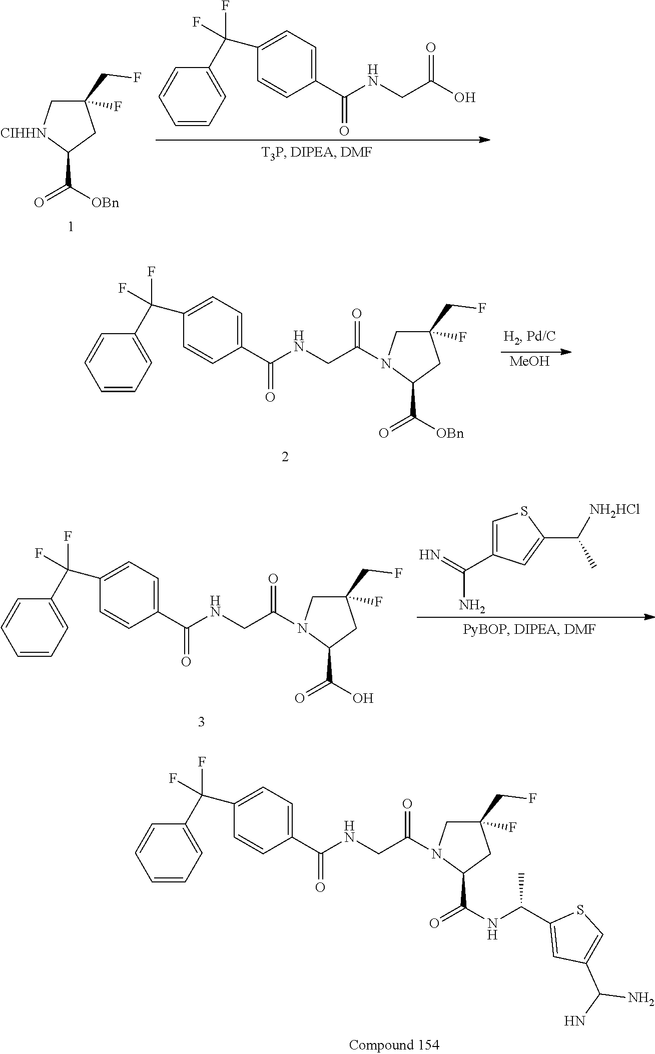

Compound 154 was prepared as a white solid from benzyl (2S,4R)-4-fluoro-4-(fluoromethyl)pyrrolidine-2-carboxylate hydrochloride, (9,9-dimethyl-9H-fluorene-3-carbonyl)glycine, and (R)-5-(1-aminoethyl) thiophene-3-carboximidamide hydrochloride based on the procedures set forth in Scheme 12. ¹H NMR (400 MHz, CD₃OD) δ 8.55 (s, 1H), 8.19 (d, J=1.5 Hz, 1H), 7.93 (d, J=8.4 Hz, 2H), 7.63 (d, J=8.3 Hz, 2H), 7.53-7.50 (m, 3H), 7.47 (d, J=5.4 Hz, 3H), 5.26 (q, J=6.7 Hz, 1H), 4.76 (d, J=8.4 Hz, 1H), 4.68-4.61 (m, 2H), 4.32 (d, J=16.7 Hz, 1H), 4.13 (dd, J=17.0, 10.6 Hz, 2H), 3.94 (dd, J=32.1, 12.2 Hz, 1H), 2.59 (dd, J=23.6, 15.3 Hz, 1H), 2.28-2.14 (m, 1H), 1.60 (t, J=14.9 Hz, 3H). LCMS (ESI) (m/z): 604 (M+H)⁺.

### Scheme 150: (S)—N—((R)-1-(4-carbamimidoylthiophen-2-yl)ethyl)-7-((2′,4′-difluoro-[1,1′-biphenyl]-4-carbonyl)glycyl)-1,4-dioxa-7-azaspiro[4.4]nonane-8-carboxamide (Compound 158)

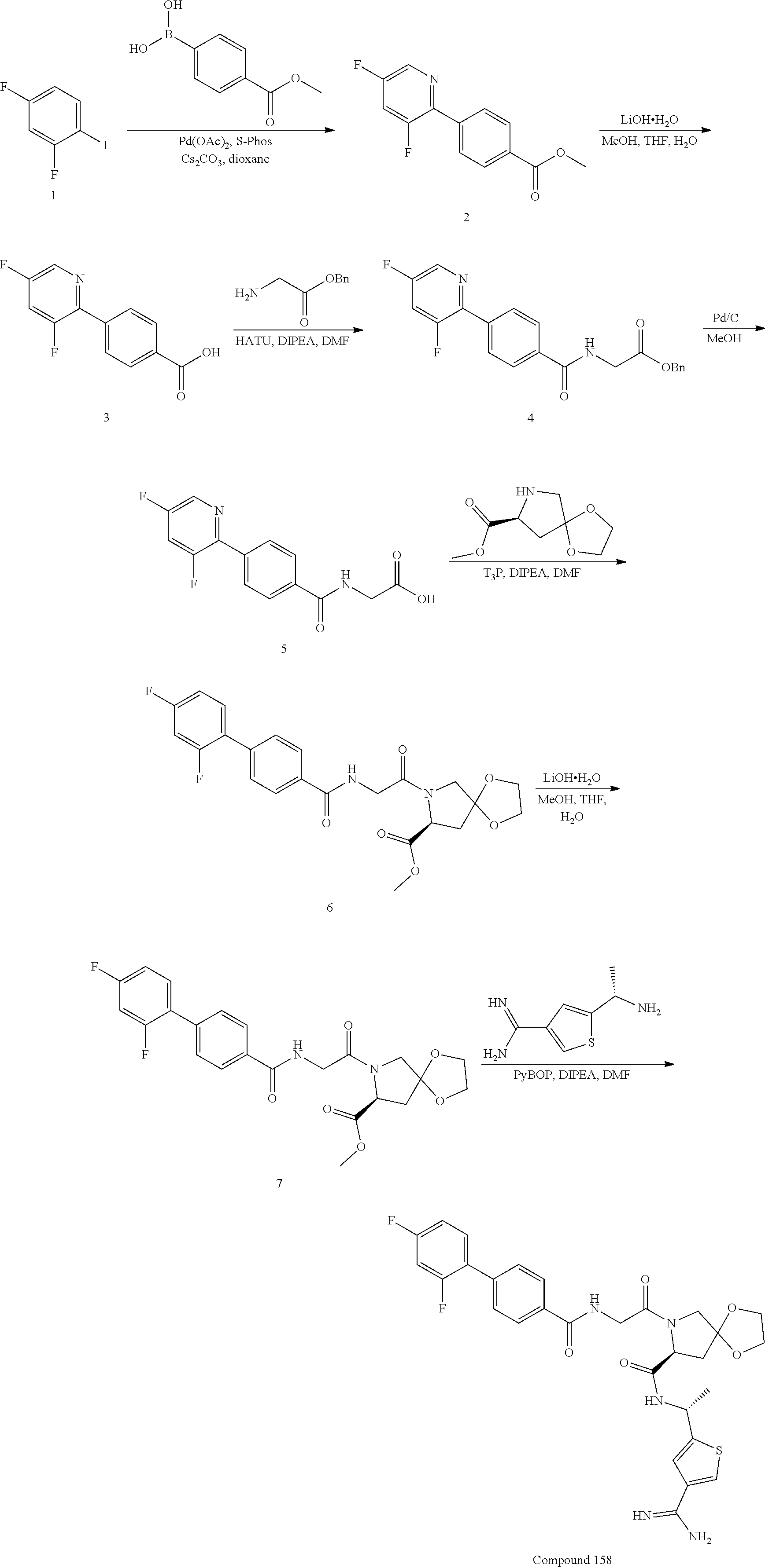

### Step 1: methyl 2,4′-difluoro-[1,1′-biphenyl]-4-carboxylate (2)

To a mixture of 1,4-difluoro-2-iodobenzene (1.0 g, 4.17 mmol) and (4-(methoxycarbonyl) phenyl)boronic acid (750 mg, 4.17 mmol) in 1,4-dioxane (10 mL) and water (3 mL) was added K₂CO₃ (1.7 g, 12.5 mmol) and Pd(PPh₃)₄ (241 mg, 0.21 mmol) at 25° C. under N₂ atmosphere and the reaction mixture was stirred at 100° C. overnight. The mixture was diluted with water and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=10:1) to give methyl 2′,4′-difluoro-[1,1′-biphenyl]-4-carboxylate (470 mg, yield 45.4%) as a colorless oil. LC/MS (ESI) m/z:249 (M+H)⁺.

### Step 2: 2′,4′-difluoro-[1,1′-biphenyl]-4-carboxylic acid (3)

To a solution of methyl 2′,4′-difluoro-[1,1′-biphenyl]-4-carboxylate (470 mg, 1.89 mmol) in MeOH (6 mL), THF (3 mL) and water (3 mL) was added LiOH·H₂O (238 mg, 5.68 mmol) at 0° C. and the mixture was stirred at room temperature for 16 hours. The mixture was acidified with 1 N aq·HCl to pH˜3, extracted with EtOAc twice. The combined organic layers were washed with brine, dried over anhydrous Na₂SO₄, filtered, and concentrated under reduced pressure to give 2′,4′-difluoro-[1,1′-biphenyl]-4-carboxylic acid (430 mg, yield 96.9%) as a white solid, which was used directly in the next reaction. LC/MS (ESI) (m/z): 235 (M+H)⁺.

### Step 3: benzyl (2,4′-difluoro-[1,1′-biphenyl]-4-carbonyl)glycinate (4)

To a mixture of 2′,4′-difluoro-[1,1′-biphenyl]-4-carboxylic acid (430 mg, 1.84 mmol) and benzyl glycinate (455 mg, 2.75 mmol) in DMF (6 mL) was added HBTU (1.0 g, 2.75 mmol) and DIPEA (1.2 g, 9.18 mmol) under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography on silica gel (PE:EtOAc=3:1) to give benzyl (2′,4′-difluoro-[1,1′-biphenyl]-4-carbonyl)glycinate (470 mg, yield 67.1%) as a colorless oil. LC/MS (ESI) m/z: 382 (M+H)⁺.

### Step 4: (2′,4′-difluoro-[1,1′-biphenyl]-4-carbonyl)glycine (5)

To a solution of benzyl (2′,4′-difluoro-[1,1′-biphenyl]-4-carbonyl)glycinate (470 mg, 1.23 mmol) in MeOH (5 mL). Pd/C, 10% wt. (131 mg, 0.123 mmol). The flask was evacuated and backfilled with H₂ gas in a balloon. The reaction was stirred at room temperature overnight. The reaction mixture was filtered through Celited pad. The filtrate was concentracted to give a white solid. as a white solid, which was used directly in the next reaction. LC/MS (ESI) (m/z): 292 (M+H)⁺.

### Step 5: methyl (S)-7-((2′,4′-difluoro-[1,1′-biphenyl]-4-carbonyl)glycyl)-1,4-dioxa-7-azaspiro[4.4]nonane-8-carboxylate (6)

To a mixture of (2,4′-difluoro-[1,1′-biphenyl]-4-carbonyl)glycine (93 mg, 0.32 mmol) and methyl (S)-1,4-dioxa-7-azaspiro[4.4]nonane-8-carboxylate (60 mg, 0.32 mmol) in DMF (2 mL) was added DIPEA (207 mg, 1.60 mmol) and T₃P (306 mg, 0.48 mmol, 50% wt. in EtOAc) under N₂ atmosphere and the mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography on silica gel (DCM:MeOH=20:1) to give methyl (S)-7-((2′,4′-difluoro-[1,1′-biphenyl]-4-carbonyl)glycyl)-1,4-dioxa-7-azaspiro[4.4]nonane-8-carboxylate (126 mg, yield 85.4%) as a colorless oil. LC/MS (ESI) m/z: 461 (M+H)⁺.

### Step 6: (S)-7-((2,4′-difluoro-[1,1′-biphenyl]-4-carbonyl)glycyl)-1,4-dioxa-7-azaspiro[4.4]nonane-6-carboxylic acid (7)

To a solution of methyl (S)-7-((2′,4′-difluoro-[1,1′-biphenyl]-4-carbonyl)glycyl)-1,4-dioxa-7-azaspiro[4.4]nonane-8-carboxylate (120 mg, 0.26 mmol) in MeOH (2 mL), THF (1 mL) and water (1 mL) was added LiOH·H₂O (33 mg, 0.78 mmol) at 0° C. and the mixture was stirred at room temperature for 2 hours. The mixture was acidified with 1 N aq·HCl to pH-4, extracted with EtOAc twice. The combined organic layers were washed with brine, dried over anhydrous Na₂SO₄, filtered, and concentrated under reduced pressure to give ((S)-7-((2′,4′-difluoro-[1,1′-biphenyl]-4-carbonyl)glycyl)-1,4-dioxa-7-azaspiro[4.4]nonane-8-carboxylic acid (100 mg, yield 85.9%) as a colorless oil, which was used directly in the next reaction. LC/MS (ESI) (m/z): 447 (M+H)⁺.

### Step 7: (S)—N—((R)-1-(4-carbamimidoylthiophen-2-yl)ethyl)-7-((2′,4′-difluoro-[1,1′-biphenyl]-4-carbonyl)glycyl)-1,4-dioxa-7-azaspiro[4.4]nonane-6-carboxamide (Compound 158)

To a mixture of ((S)-7-((2′,4′-difluoro-[1,1′-biphenyl]-4-carbonyl)glycyl)-1,4-dioxa-7-azaspiro[4.4]nonane-8-carboxylic acid (50 mg, 0.11 mmol) and (S)-5-(1-aminoethyl) thiophene-3-carboximidamide (23 mg, 0.13 mmol) in DMF (2 mL) was added DIPEA (72 mg, 0.56 mmol) and PyBOP (87 mg, 0.17 mmol) at 0° C. under N₂ atmosphere and the mixture was stirred at 25° C. for 1 hour. The reaction mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (DCM:MeOH=10:1) and prep-HPLC to give Compound 158 (2.0 mg, yield 3.0%) as a white solid. ¹H NMR (400 MHz, CD₃OD) δ 8.54 (s, 1H), 8.24 (t, J=5.6 Hz, 1H), 7.94 (d, J=8.4 Hz, 2H), 7.64 (d, J=7.1 Hz, 2H), 7.56 (dd, J=17.6, 11.0 Hz, 2H), 7.09 (t, J=9.3 Hz, 2H), 5.28 (q, J=7.1 Hz, 1H), 4.56 (s, 1H), 4.19 (s, 2H), 4.02-3.94 (m, 4H), 3.80 (q, J=10.8 Hz, 2H), 2.44 (dd, J=12.9, 9.1 Hz, 1H), 2.21 (dd, J=13.1, 6.4 Hz, 1H), 1.61 (t, J=18.4 Hz, 3H). LC/MS (ESI) m/z: 598 (M+H)⁺.

### Scheme 151: Synthesis of (2S,4R)—N—((R)-1-(4-carbamimidoylthiophen-2-yl)ethyl)-1-((9,9-difluoro-9H-fluorene-3-carbonyl)glycyl)-4-(trifluoromethoxy)pyrrolidine-2-carboxamide(Compound 159)

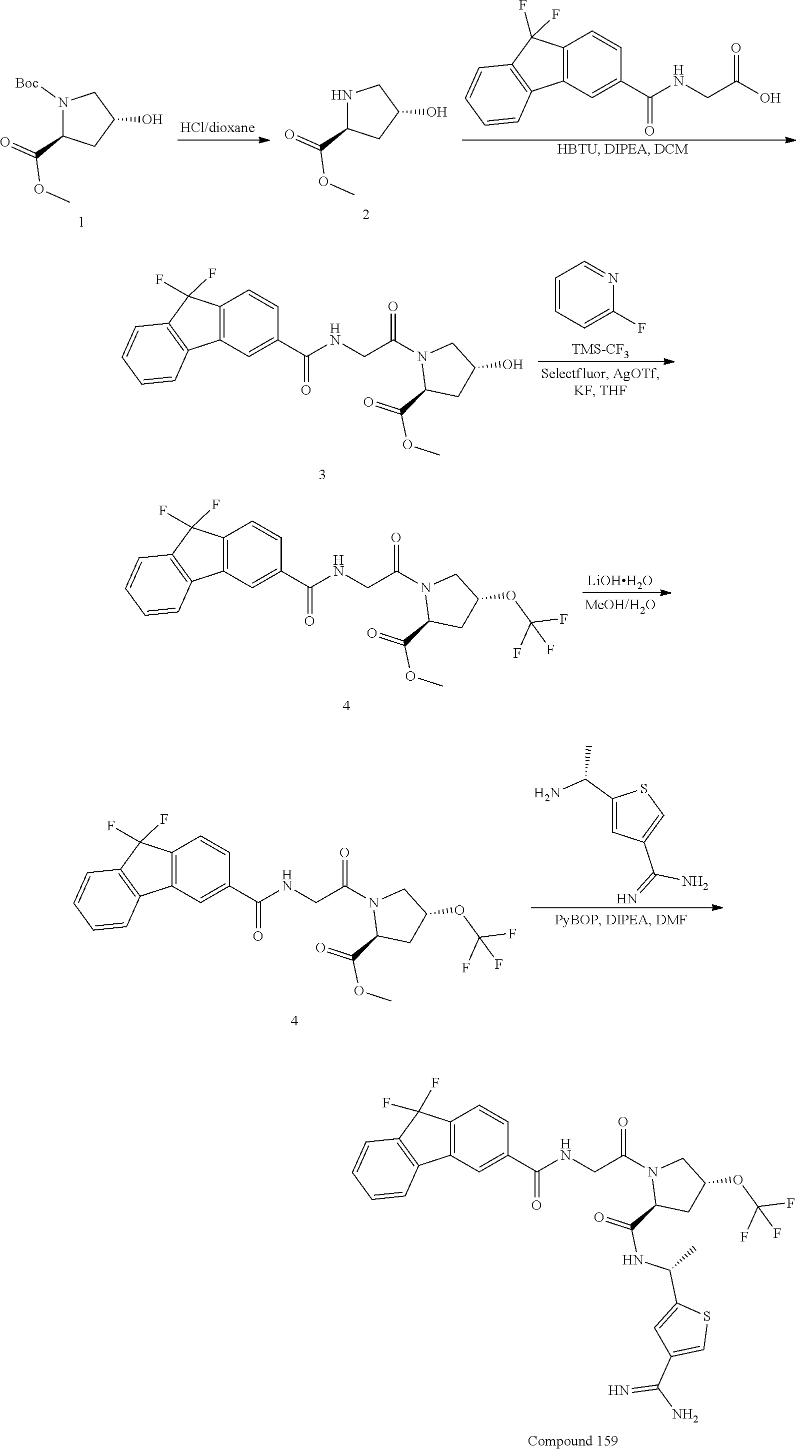

### Step 1: methyl (2S,4R)-4-hydroxypyrrolidine-2-carboxylate (2)

To a solution of 1-(tert-butyl) 2-methyl (2S,4R)-4-hydroxypyrrolidine-1,2-dicarboxylate (200 mg, 118 mmol) in HCl/1,4-dioxane (2.5 mL) was stirred under N₂ atmosphere at room temperature for 2 hours. The reaction mixture was concentrated to dryness under reduced pressure, diluted with DCM again and dried under vacuum to give methyl (2S,4R)-4-hydroxypyrrolidine-2-carboxylate (115 mg, yield 96.7%), which was used directly in the next step without further purification. LC/MS (ESI) m/z: 146 (M+H)⁺.

### Step 2: methyl (2S,4R)-1-((9,9-difluoro-9H-fluorene-3-carbonyl)glycyl)-4-hydroxypyrrolidine-2-carboxylate (3)

To a mixture of methyl (2S,4R)-4-hydroxypyrrolidine-2-carboxylate (115 mg, 0.79 mmol) and (9,9-difluoro-9H-fluorene-3-carbonyl)glycine (248 mg, 0.82 mmol) in DMF (1.5 mL) was added DIPEA (630 mg, 4.9 mmol) and HBTU (417 mg, 1.1 mmol) at 0° C. under N₂ atmosphere and the mixture was stirred at 25° C. for 2 hours. The mixture was diluted with water, extracted with EtOAc twice. The combined organic layers were dried over anhydrous Na₂SO₄, filtered, and concentrated to dryness under reduced pressure. The residue was purified by column chromatography on silica gel (PE:EtOAc=2:1) to give methyl (2S,4R)-1-((9,9-difluoro-9H-fluorene-3-carbonyl)glycyl)-4-hydroxypyrrolidine-2-carboxylate (290 mg, yield 85.4%) as a colorless oil. LC/MS (ESI) m/z: 431 (M+H)⁺.

### Step 3: methyl (2S,4R)-1-((9,9-difluoro-9H-fluorene-3-carbonyl)glycyl)-4-(trifluoromethoxy) pyrrolidine-2-carboxylate (4)

To a solution of methyl (2S,4R)-1-((9,9-difluoro-9H-fluorene-3-carbonyl)glycyl)-4-hydroxypyrrolidine-2-carboxylate (290 mg, 0.68 mmol) in EtOAc (5 mL) was added selectfluor (1.2 g, 3.4 mmol), AgOTf (1.7 g, 6.7 mmol), KF (388 mg, 6.7 mmol), 2-fluoropyridine (650 mg, 6.7 mmol) and TMSCF₃ (950 mg, 6.7 mmol) under N₂ atmosphere at 40° C. and stirred for 48 hours. The mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (PE:EtOAc=2:1) to give methyl (2S,4R)-1-((9,9-difluoro-9H-fluorene-3-carbonyl)glycyl)-4-(trifluoromethoxy)pyrrolidine-2-carboxylate (110 mg, yield 32.5%) as a yellow oil. LCMS (ESI) m/z=499 (M+H)⁺.

### Step 4: (2S,4R)-1-((9,9-difluoro-9H-fluorene-3-carbonyl)glycyl)-4-(trifluoromethoxy) pyrrolidine-2-carboxylic acid (5)

To a solution of methyl (2S,4R)-1-((9,9-difluoro-9H-fluorene-3-carbonyl)glycyl)-4-(trifluoromethoxy)pyrrolidine-2-carboxylate (50 mg, 0.10 mmol) in MeOH (0.6 mL) and water (0.3 mL) was added LiOH·H₂O (25 mg, 0.6 mmol) at 0° C. and the mixture was stirred at room temperature for 2 hours. The mixture was acidified with 1 N aq·HCl to pH-3, extracted with EtOAc twice. The combined organic layers were washed with brine, dried over anhydrous Na₂SO₄, filtered, and concentrated under reduced pressure to dryness to give (2S,4R)-1-((9,9-difluoro-9H-fluorene-3-carbonyl)glycyl)-4-(trifluoromethoxy)pyrrolidine-2-carboxylic acid (30 mg, yield 62.0%) as a yellow oil, which was used directly in the next step. LC/MS (ESI) (m/z): 485 (M+H)⁺.

### Step 5: (2S,4R)—N—((R)-1-(4-carbamimidoylthiophen-2-yl)ethyl)-1-((9,9-difluoro-9H-fluorene-3-carbonyl)glycyl)-4-(trifluoromethoxy)pyrrolidine-2-carboxamide(Compound 162)

To a mixture of (2S,4R)-1-((9,9-difluoro-9H-fluorene-3-carbonyl)glycyl)-4-(trifluoromethoxy)pyrrolidine-2-carboxylic acid (30 mg, 0.062 mmol) and (R)-5-(1-aminoethyl)thiophene-3-carboximidamide (11 mg, 0.062 mmol) in DMF (2 mL) was added DIPEA (48 mg, 0.37 mmol) and PyBOP (40 mg, 0.074 mmol) at 0° C. under N₂ atmosphere and the mixture was stirred at 25° C. for 2 hours. The mixture was diluted with water, extracted with EtOAc twice. The combined organic layers were dried over anhydrous Na₂SO₄, filtered, and concentrated to dryness under reduced pressure. The residue was purified by column chromatography on silica gel (DCM:MeOH=12:1) and prep-HPLC to give Compound 162 (5 mg, yield 12.7%) as a white solid. ¹H NMR (400 MHz, CD₃OD) δ 8.54 (s, 1H), 8.26-8.15 (m, 2H), 7.87 (t, J=9.8 Hz, 1H), 7.74 (dd, J=17.0, 7.6 Hz, 2H), 7.65 (d, J=7.3 Hz, 1H), 7.56 (dd, J=18.8, 11.4 Hz, 2H), 7.45 (t, J=7.6 Hz, 1H), 5.37-5.20 (m, 2H), 4.61 (d, J=7.8 Hz, 1H), 4.34 (d, J=16.8 Hz, 1H), 4.20 (d, J=16.8 Hz, 1H), 4.02 (s, 2H), 2.64-2.51 (m, 1H), 2.35-2.27 (m, 1H), 1.61 (t, J=16.0 Hz, 3H). LC/MS (ESI) m/z: 636 (M+H)⁺.

### Scheme 152: Synthesis of (2S,4R*)—N—((R)-1-(4-carbamimidoylthiophen-2-yl)ethyl)-4-(1,3-dioxan-2-yl)-1-((4-(4-fluorophenoxy)benzoyl)glycyl)pyrrolidine-2-carboxamide (Compound 160)

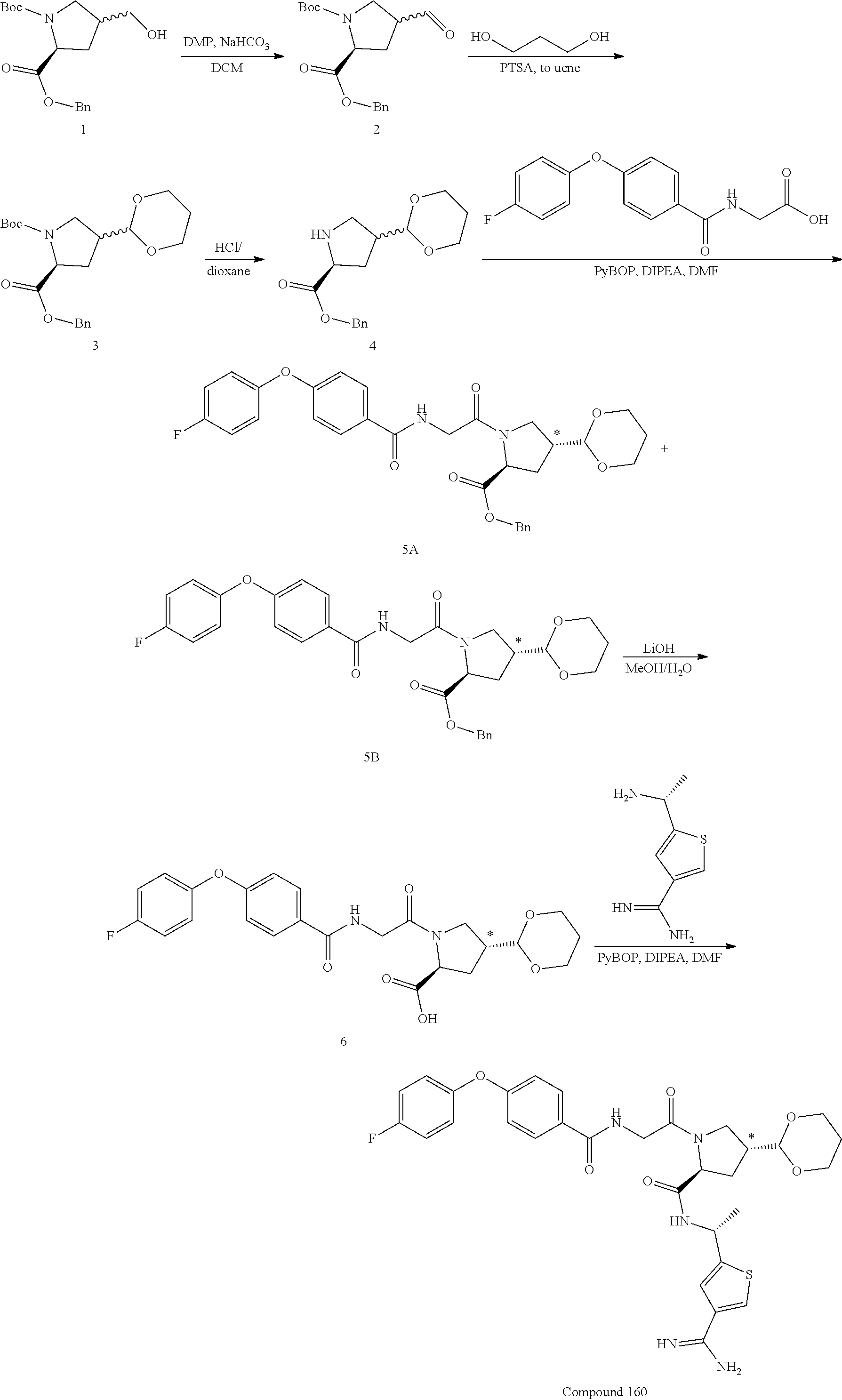

### Step 1: 2-benzyl 1-(tert-butyl) (2S)-4-formylpyrrolidine-1,2-dicarboxylate (2)

To a solution of 2-benzyl 1-(tert-butyl) (2S)-4-(hydroxymethyl)pyrrolidine-1,2-dicarboxylate (410 mg, 1.2 mmol) in DCM (4 mL) was added NaHCO₃ (130 mg, 1.2 mmol) and DMP (670 mg, 1.6 mmol) under N₂ atmosphere at room temperature and stirred overnight. The mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (PE:EtOAc=4:1) to give 2-benzyl 1-(tert-butyl) (2S)-4-formylpyrrolidine-1,2-dicarboxylate (300 mg, yield 73.8%) as a white solid. LCMS (ESI) m/z=334 (M+H)⁺.

### Step 2: 2-benzyl 1-(tert-butyl) (2S)-4-(1,3-dioxan-2-yl)pyrrolidine-1,2-dicarboxylate (3)

To a solution of 2-benzyl 1-(tert-butyl) (2S)-4-formylpyrrolidine-1,2-dicarboxylate (300 mg, 0.9 mmol) in toluene (7 mL) was added propane-1,3-diol (340 mg, 4.5 mmol) and PTSA (10 mg, 0.045 mmol) under N₂ atmosphere at 140° C. and stirred for 4 hours. The mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (PE:EtOAc=10:1) to give 2-benzyl 1-(tert-butyl) (2S)-4-(1,3-dioxan-2-yl)pyrrolidine-1,2-dicarboxylate (190 mg, yield 54.4%) as a white solid. LCMS (ESI) m/z=392 (M+H)⁺.

### Step 3: benzyl (2S)-4-(1,3-dioxan-2-yl)pyrrolidine-2-carboxylate (4)

To a solution of 2-benzyl 1-(tert-butyl) (2S)-4-(1,3-dioxan-2-yl)pyrrolidine-1,2-dicarboxylate (120 mg, 0.30 mmol) in HCl/1,4-dioxane (2 mL) was stirred under N₂ atmosphere at room temperature for 30 minutes. The reaction mixture was concentrated to dryness under reduced pressure, diluted with DCM again and dried under vacuum to give benzyl (2S)-4-(1,3-dioxan-2-yl)pyrrolidine-2-carboxylate (87 mg, yield 97.7%), which was used directly in the next step without further purification. LC/MS (ESI) m/z: 292 (M+H)⁺.

### Step 4: benzyl (2S,4S)-4-(1,3-dioxan-2-yl)-1-((4-(4-fluorophenoxy)benzoyl)glycyl)pyrrolidine-2-carboxylate (5B) and benzyl (2S,4R)-4-(1,3-dioxan-2-yl)-1-((4-(4-fluorophenoxy)benzoyl)glycyl)pyrrolidine-2-carboxylate (5A)

To a mixture of benzyl (2S)-4-(1,3-dioxan-2-yl)pyrrolidine-2-carboxylate (90 mg, 0.30 mmol) and (4-(4-fluorophenoxy)benzoyl)glycine (87 mg, 0.30 mmol) in DMF (2 mL) was added DIPEA (193 mg, 1.5 mmol) and PyBOP (156 mg, 0.3 mmol) at 0° C. under N₂ atmosphere and the mixture was stirred at 25° C. for 1 hour. The mixture was diluted with EtOAc and washed with water twice. The organic layers were dried over anhydrous Na₂SO₄, filtered, and concentrated to dryness under reduced pressure. The residue was purified by column chromatography on silica gel (PE:EtOAc=2:1) and SFC to give benzyl (2S,4R)—N—((R)-1-(4-carbamimidoylthiophen-2-yl)ethyl)-4-(1,3-dioxan-2-yl)-1-((4-(4 fluorophenoxy)benzoyl)glycyl)pyrrolidine-2-carboxylate, 5A (50 mg, yield 30.0%) and benzyl (2S,4S)—N—((R)-1-(4-carbamimidoylthiophen-2-yl)ethyl)-4-(1,3-dioxan-2-yl)-1-((4-(4-fluorophenoxy)benzoyl)glycyl)pyrrolidine-2-carboxylate, 5B (60 mg, 35.6%). LC/MS (ESI) m/z: 563 (M+H)⁺.

### Step 5: (2S,4R*)-4-(1,3-dioxan-2-yl)-1-((4-(4-fluorophenoxy)benzoyl)glycyl)pyrrolidine-2-carboxylic acid (6)

To a solution of benzyl (2S,4R*)-4-(1,3-dioxan-2-yl)-1-((4-(4-fluorophenoxy)benzoyl)glycyl) pyrrolidine-2-carboxylate (50 mg, 0.089 mmol) in MeOH (1.0 mL) and water (0.5 mL) was added LiOH (22 mg, 0.53 mmol) at 0° C. and the mixture was stirred at room temperature for 1 hour. The mixture was acidified with 1 N aq·HCl to pH˜3, extracted with EtOAc twice. The combined organic layers were washed with brine, dried over anhydrous Na₂SO₄, filtered, and concentrated under reduced pressure to dryness to give (2S,4R*)-4-(1,3-dioxan-2-yl)-1-((4-(4-fluorophenoxy)benzoyl)glycyl)pyrrolidine-2-carboxylic acid (39 mg, yield 92.8%) as a colorless oil, which was used directly in the next step. LCMS (ESI) (m/z): 473 (M+H)⁺.

### Step 6: (2S,4R*)—N—((R)-1-(4-carbamimidoylthiophen-2-yl)ethyl)-4-(1,3-dioxan-2-yl)-1-((4-(4-fluorophenoxy)benzoyl)glycyl)pyrrolidine-2-carboxamide (Compound 160)

To a mixture of (2S,4R*)-4-(1,3-dioxan-2-yl)-1-((4-(4-fluorophenoxy)benzoyl)glycyl)pyrrolidine-2-carboxylic acid (39 mg, 0.083 mmol) and (R)-5-(1-aminoethyl)thiophene-3-carboximidamide (29 mg, 0.17 mmol) in DMF (1 mL) was added DIPEA (64 mg, 0.50 mmol) and PyBOP (47 mg, 0.091 mmol) at 0° C. under N₂ atmosphere and the mixture was stirred at 25° C. for 1 hour. The mixture was diluted with EtOAc and washed with water twice. The organic layers were dried over anhydrous Na₂SO₄, filtered, and concentrated to dryness under reduced pressure. The residue was purified by column chromatography on silica gel (DCM:MeOH=9:1) and prep-HPLC to give Compound 160 (8 mg, 15.5%). ¹H NMR (400 MHz, CD₃OD) δ 8.54 (s, 1H), 8.23 (d, J=4.7 Hz, 1H), 7.84 (t, J=7.7 Hz, 2H), 7.49 (d, J=14.4 Hz, 1H), 7.16 (t, J=8.7 Hz, 2H), 7.11-7.06 (m, 2H), 6.99 (d, J=8.8 Hz, 2H), 5.25 (q, J=6.8 Hz, 1H), 4.54 (d, J=6.4 Hz, 1H), 4.19-4.04 (m, 4H), 3.81 (t, J=10.3 Hz, 3H), 3.66-3.59 (m, 1H), 2.64 (d, J=7.7 Hz, 1H), 2.22 (dd, J=9.4, 3.7 Hz, 1H), 2.08-2.01 (m, 2H), 1.59 (t, J=15.6 Hz, 3H), 1.38 (d, J=14.0 Hz, 2H). LC/MS (ESI) m/z: 624 (M+H)⁺.

### Scheme 153: Synthesis of (2S,4S*)—N—((R)-1-(4-carbamimidoylthiophen-2-yl)ethyl)-4-(1,3-dioxan-2-yl)-1-((4-(4-fluorophenoxy)benzoyl)glycyl)pyrrolidine-2-carboxamide (Compound 161)

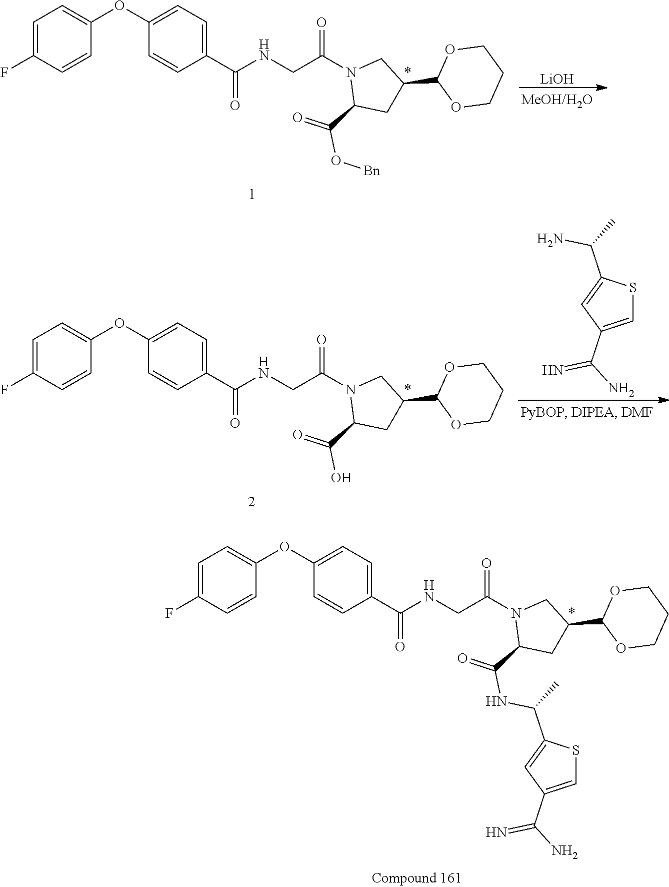

Compound 161 was prepared as a white solid from benzyl (2S,4S*)-4-(1,3-dioxan-2-yl)-1-((4-(4-fluorophenoxy)benzoyl)glycyl)pyrrolidine-2-carboxylate and (R)-5-(1-aminoethyl)thiophene-3-carboximidamide based on the procedures set forth in Scheme 3. ¹H NMR (400 MHz, CD₃OD) δ 8.54 (s, 1H), 8.23 (t, J=4.4 Hz, 1H), 7.85 (d, J=8.8 Hz, 2H), 7.53 (s, 1H), 7.16 (t, J=8.7 Hz, 2H), 7.10-7.06 (m, 2H), 6.99 (d, J=8.8 Hz, 2H), 5.26 (q, J=6.6 Hz, 1H), 4.54 (d, J=6.3 Hz, 1H), 4.40 (t, J=8.5 Hz, 1H), 4.26 (d, J=16.6 Hz, 1H), 4.11 (s, 1H), 4.04 (d, J=4.2 Hz, 1H), 3.91-3.69 (m, 4H), 3.56 (t, J=9.8 Hz, 1H), 2.58-2.51 (m, 1H), 2.36 (dd, J=14.3, 6.5 Hz, 1H), 1.93-1.85 (m, 1H), 1.60 (t, J=14.8 Hz, 3H), 1.36 (d, J=18.7 Hz, 2H). LC/MS (ESI) m/z: 624 (M+H)⁺.

### Scheme 154: Synthesis of (8S)—N—((R)-1-(4-carbamimidoylthiophen-2-yl)ethyl)-7-((9-hydroxy-9H-fluorene-3-carbonyl)glycyl)-1,4-dioxa-7-azaspiro[4.4]nonane-8-carboxamide (Compound 162)

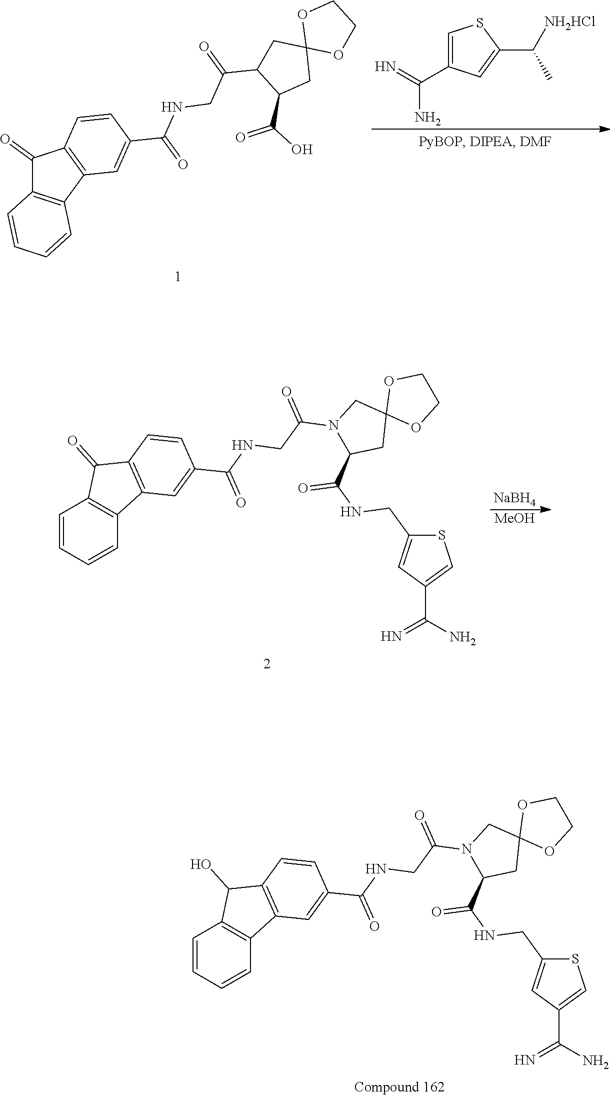

### Step 1: (S)—N—((R)-1-(4-carbamimidoylthiophen-2-yl)ethyl)-7-((9-oxo-9H-fluorene-3-carbonyl) glycyl)-1,4-dioxa-7-azaspiro[4.4]nonane-8-carboxamide (2)

To a mixture of (S)-7-((9-oxo-9H-fluorene-3-carbonyl)glycyl)-1,4-dioxa-7-azaspiro[4.4]nonane-8-carboxylic acid (120 mg, 0.28 mmol) and (R)-5-(1-aminoethyl) thiophene-3-carboximidamide hydrochloride (94 mg, 0.55 mmol) in DMF (2 mL) was added DIPEA (178 mg, 1.38 mmol) and PyBOP (215 mg, 0.41 mmol) at 0° C. and the mixture was stirred under N₂ atmosphere at room temperature for 1 hour. The reaction mixture was diluted with water and extracted with EtOAc twice. The combined organic layers were washed with brine, dried over anhydrous Na₂SO₄, filtered, and concentrated to dryness under reduced pressure. The residue was purified by prep-TLC (DCM:MeOH=10:1) to give (S)—N—((R)-1-(4-carbamimidoylthiophen-2-yl)ethyl)-7-((9-oxo-9H-fluorene-3-carbonyl)glycyl)-1,4-dioxa-7-azaspiro[4.4]nonane-8-carboxamide (111 mg, yield 67.3%) as a white solid. LCMS (ESI) (m/z): 588 (M+H)⁺.

### Step 2: (8S)—N—((R)-1-(4-carbamimidoylthiophen-2-yl)ethyl)-7-((9-hydroxy-9H-fluorene-3-carbonyl)glycyl)-1,4-dioxa-7-azaspiro[4.4]nonane-6-carboxamide (Compound 162)

To a solution of (S)—N—((R)-1-(4-carbamimidoylthiophen-2-yl)ethyl)-7-((9-oxo-9H-fluorene-3-carbonyl)glycyl)-1,4-dioxa-7-azaspiro[4.4]nonane-8-carboxamide (30 mg, 0.051 mmol) in MeOH (1.0 mL) was added NaBH₄ (5 mg, 0.13 mmol) at 0° C. and the mixture was stirred at 0° C. for 1 hour. The reaction mixture was quenched with ice-water and purified by prep-HPLC to give Compound 162 (10.9 mg, yield 36.2%) as a white solid. ¹H NMR (400 MHz, CD₃OD) δ 8.19 (s, 1H), 7.94 (t, J=16.2 Hz, 1H), 7.81 (d, J=7.7 Hz, 1H), 7.75 (s, 1H), 7.68 (d, J=4.9 Hz, 1H), 7.63 (d, J=7.3 Hz, 1H), 7.47 (d, J=1.1 Hz, 1H), 7.42 (t, J=7.2 Hz, 1H), 7.36 (t, J=7.4 Hz, 1H), 5.58 (s, 1H), 5.28 (q, J=6.7 Hz, 1H), 4.59 (dd, J=8.8, 6.5 Hz, 1H), 4.19 (d, J=4.5 Hz, 1H), 4.06-3.90 (m, 5H), 3.79 (d, J=11.2 Hz, 2H), 2.43 (dd, J=13.1, 9.0 Hz, 1H), 2.22 (dd, J=13.2, 6.3 Hz, 1H), 1.60 (dd, J=36.7, 6.9 Hz, 3H). LC/MS (ESI) m/z: 590 (M+H)⁺.

### Scheme 155: Synthesis of (S)—N—((R)-1-(4-carbamimidoylthiophen-2-yl)ethyl)-7-((5-(4-fluorophenyl)picolinoyl)glycyl)-1,4-dioxa-7-azaspiro[4.4]nonane-8-carboxamide (Compound 163)

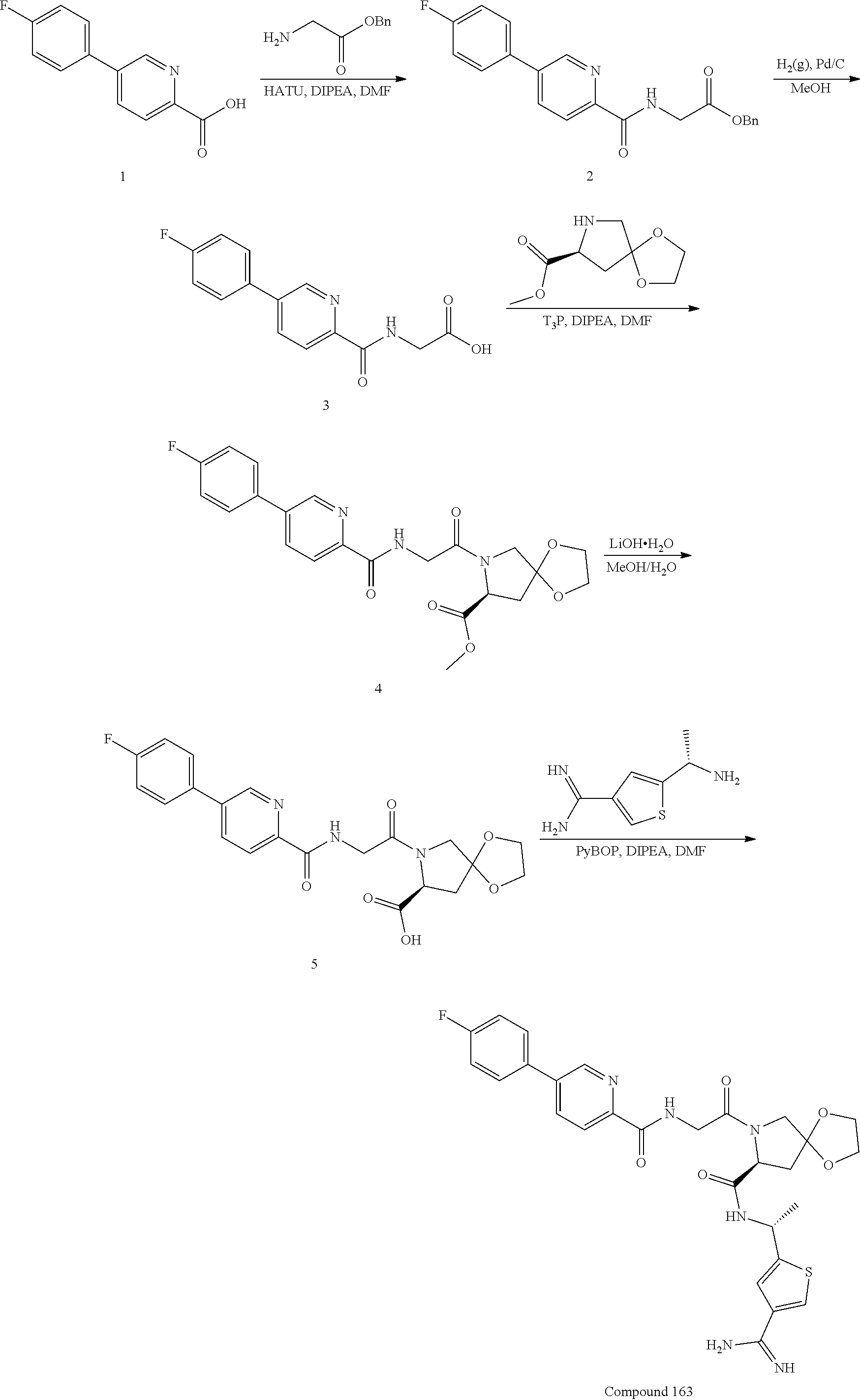

Compound 163 was prepared as a white solid from 5-(4-fluorophenyl)picolinic acid, benzyl glycinate HCl salt, methyl (S)-1,4-dioxa-7-azaspiro[4.4]nonane-8-carboxylate, and (S)-5-(1-aminoethyl)thiophene-3-carboximidamide HCl salt based on the procedures set forth in Scheme 150. ¹H NMR (400 MHz, CD₃OD) δ 8.91 (d, J=1.6 Hz, 1H), 8.53 (s, 1H), 8.26-8.13 (m, 3H), 7.79-7.74 (m, 2H), 7.55 (d, J=7.3 Hz, 1H), 7.27 (t, J=8.7 Hz, 2H), 5.31 (dq, J=13.8, 6.9 Hz, 1H), 4.62-4.54 (m, 1H), 4.35-4.16 (m, 2H), 4.04-3.95 (m, 4H), 3.77 (dd, J=23.4, 10.6 Hz, 2H), 2.44 (dd, J=13.0, 9.0 Hz, 1H), 2.21 (dd, J=13.2, 6.8 Hz, 1H), 1.63 (dd, J=31.0, 7.0 Hz, 3H). LC/MS (ESI) m/z: 581 (M+H)⁺.

### Scheme 156: Synthesis of (S)—N—((R)-1-(4-carbamimidoylthiophen-2-yl)ethyl)-7-((4-(5-fluoropyridin-2-yl)benzoyl)glycyl)-1,4-dioxa-7-azaspiro[4.4]nonane-8-carboxamide (Compound 164)

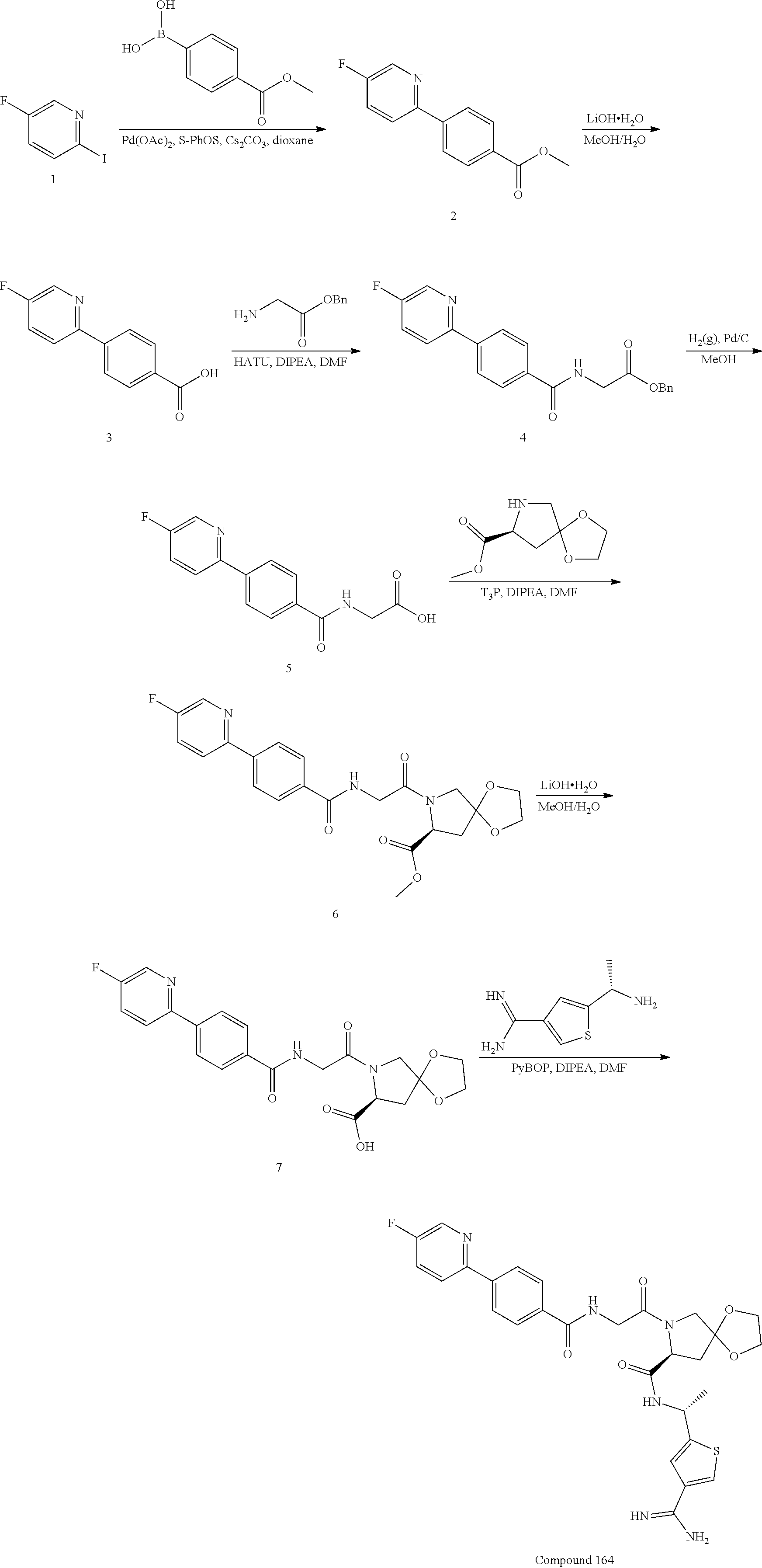

Compound 164 was prepared as a white solid from 5-fluoro-2-iodopyridine, (4-(methoxycarbonyl)phenyl)boronic acid, benzyl glycinate HCl salt, methyl (S)-1,4-dioxa-7-azaspiro[4.4]nonane-8-carboxylate, and (S)-5-(1-aminoethyl)thiophene-3-carboximidamide HCl salt based on the procedures set forth in Scheme 150. ¹H NMR (400 MHz, CD₃OD) δ 8.57 (d, J=2.7 Hz, 1H), 8.54 (s, 1H), 8.24 (t, J=5.5 Hz, 1H), 8.09 (d, J=8.4 Hz, 2H), 8.02-7.94 (m, 3H), 7.71 (td, J=8.6, 2.8 Hz, 1H), 7.54 (d, J=6.6 Hz, 1H), 5.39-5.23 (m, 1H), 4.64-4.53 (m, 1H), 4.16 (dd, J=25.4, 8.7 Hz, 2H), 4.03-3.94 (m, 4H), 3.80 (q, J=10.7 Hz, 2H), 2.43 (dd, J=13.0, 8.9 Hz, 1H), 2.21 (dd, J=13.1, 6.5 Hz, 1H), 1.63 (dd, J=37.5, 7.0 Hz, 3H). LC/MS (ESI) m/z: 581 (M+H)⁺.

### Scheme 157: Synthesis of (S)—N—((R)-1-(4-carbamimidoylthiophen-2-yl)ethyl)-7-((2′,5′-difluoro-[1,1′-biphenyl]-4-carbonyl)glycyl)-1,4-dioxa-7-azaspiro[4.4]nonane-8-carboxamide (Compound 166)

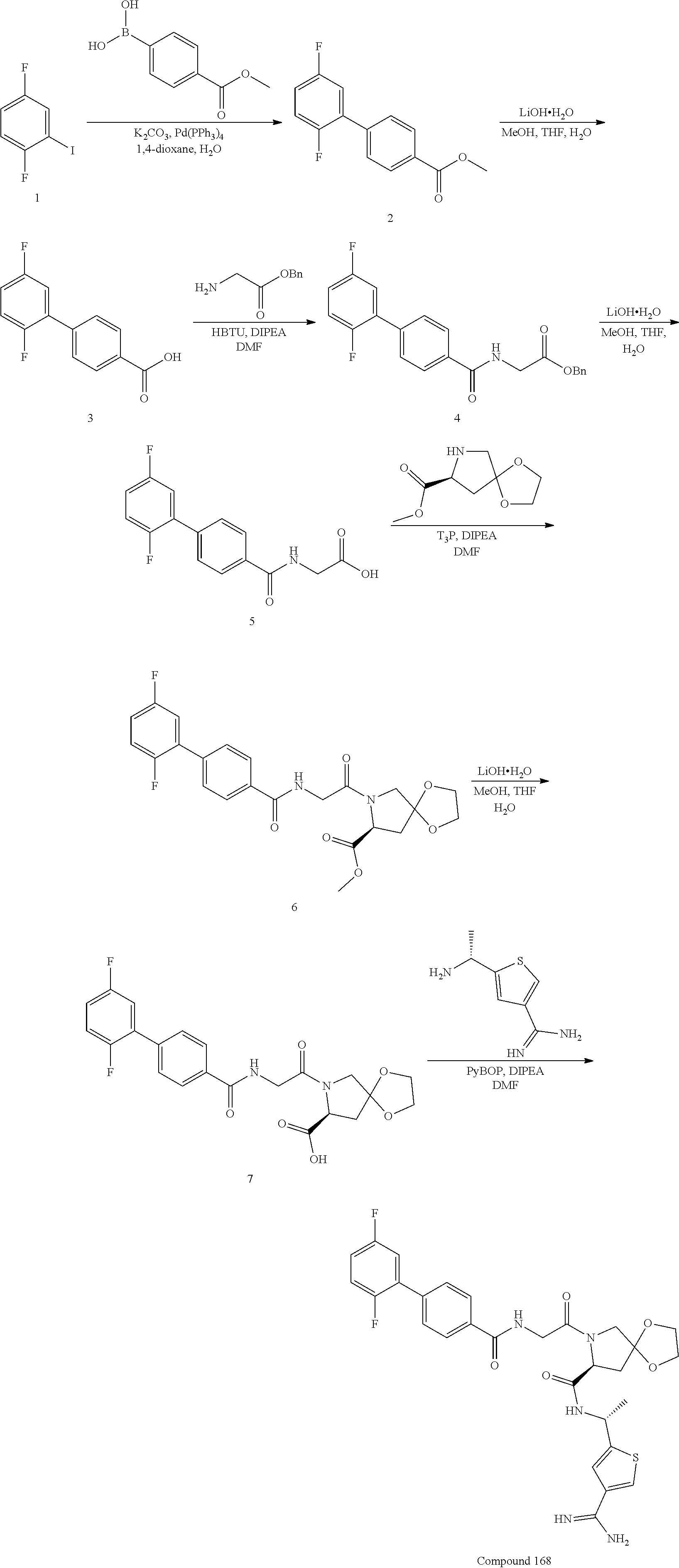

### Step 1: methyl 2′,5′-difluoro-[1,1′-biphenyl]-4-carboxylate (2)

To a mixture of 1,4-difluoro-2-iodobenzene (1.0 g, 4.17 mmol) and (4-(methoxycarbonyl)phenyl)boronic acid (750 mg, 4.17 mmol) in 1,4-dioxane (10 mL) and water (3 mL) was added K₂CO₃ (1.7 g, 12.5 mmol) and Pd(PPh₃)₄ (241 mg, 0.21 mmol) at 25° C. under N₂ atmosphere and the reaction mixture was degassed under N₂ atmosphere for three times and stirred at 100° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give methyl 2′,5′-difluoro-[1,1′-biphenyl]-4-carboxylate (510 mg, yield 49.3%) as a colorless oil.
Step 2: 2′,5′-difluoro-[1,1′-biphenyl]-4-carboxylic acid (3)

To a solution of methyl 2′,5′-difluoro-[1,1′-biphenyl]-4-carboxylate (500 mg, 2.01 mmol) in MeOH (6 mL), THF (3 mL) and water (3 mL) was added LiOH·H₂O (254 mg, 6.04 mmol) at 0° C. and the mixture was stirred at room temperature for 16 hours. The mixture was acidified with 1 N aq·HCl to pH˜3 and extracted with EtOAc twice. The combined organic layers were washed with brine, dried over anhydrous Na₂SO₄, filtered, and concentrated under reduced pressure to give 2′,5′-difluoro-[1,1′-biphenyl]-4-carboxylic acid (240 mg, yield 72.1%) as a white solid, which was used directly in the next reaction. LC/MS (ESI) (m/z): 233 (M−H)—.

### Step 3: benzyl (2,5′-difluoro-[1,1′-biphenyl]-4-carbonyl)glycinate (4)

To a mixture of 2′,5′-difluoro-[1,1′-biphenyl]-4-carboxylic acid (340 mg, 1.45 mmol) and benzyl glycinate (360 mg, 2.18 mmol) in DMF (5 mL) was added HBTU (826 mg, 2.18 mmol) and DIPEA (938 mg, 7.26 mmol) and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography flash chromatography (silica gel, 0-30% EtOAc in PE) to give benzyl (2′,5′-difluoro-[1,1′-biphenyl]-4-carbonyl)glycinate (446 mg, yield 80.6%) as a colorless oil. LC/MS (ESI) m/z: 382 (M+H)⁺.

### Step 4: (2′,5′-difluoro-[1,1′-biphenyl]-4-carbonyl)glycine (5)

To a solution of benzyl (2′,5′-difluoro-[1,1′-biphenyl]-4-carbonyl)glycinate (440 mg, 1.15 mmol) in MeOH (5 mL), THF (2.5 mL) and water (2.5 mL) was added LiOH·H₂O (145 mg, 3.46 mmol) at 0° C. and the mixture was stirred at room temperature for 2 hours. The mixture was acidified with 1 N aq·HCl to pH˜3 and extracted with EtOAc twice. The combined organic layers were washed with brine, dried over anhydrous Na₂SO₄, filtered, and concentrated under reduced pressure to give (2′,5′-difluoro-[1,1′-biphenyl]-4-carbonyl)glycine (320 mg, yield 95.2%) as a white solid, which was used directly in the next reaction. LC/MS (ESI) (m/z): 292 (M+H)⁺.

### Step 5: methyl (S)-7-((2′,5′-difluoro-[1,1′-biphenyl]-4-carbonyl)glycyl)-1,4-dioxa-7-azaspiro [4.4]nonane-8-carboxylate (6)

To a mixture of (2′,5′-difluoro-[1,1′-biphenyl]-4-carbonyl)glycine (78 mg, 0.27 mmol) and methyl (S)-1,4-dioxa-7-azaspiro[4.4]nonane-8-carboxylate (50 mg, 0.27 mmol) in DMF (2 mL) was added DIPEA (173 mg, 1.34 mmol) and T₃P (255 mg, 0.40 mmol, 50% wt. in EtOAc) under N₂ atmosphere and the mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-5% MeOH in DCM) to give methyl (S)-7-((2′,5′-difluoro-[1,1′-biphenyl]-4-carbonyl)glycyl)-1,4-dioxa-7-azaspiro[4.4]nonane-8-carboxylate (115 mg, yield 93.5%) as a colorless oil. LC/MS (ESI) m/z: 461 (M+H)⁺.

### Step 6: (S)-7-((2′5′-difluoro-[1,1′-biphenyl]-4-carbonyl)glycyl)-1,4-dioxa-7-azaspiro[4.4]nonane-6-carboxylic acid (7)

To a solution of methyl (S)-7-((2′,5′-difluoro-[1,1′-biphenyl]-4-carbonyl)glycyl)-1,4-dioxa-7-azaspiro[4.4]nonane-8-carboxylate (115 mg, 0.25 mmol) in MeOH (2 mL), THF (1 mL) and water (1 mL) was added LiOH·H₂O (31 mg, 0.75 mmol) at 0° C. and the mixture was stirred at room temperature for 2 hours. The mixture was acidified with 1 N aq·HCl to pH-4, extracted with EtOAc twice. The combined organic layers were washed with brine, dried over anhydrous Na₂SO₄, filtered, and concentrated under reduced pressure to give (S)-7-((2′,5′-difluoro-[1,1′-biphenyl]-4-carbonyl)glycyl)-1,4-dioxa-7-azaspiro[4.4]nonane-8-carboxylic acid (100 mg, yield 89.7%) as a colorless oil, which was used directly in the next reaction. LC/MS (ESI) (m/z): 447 (M+H)⁺.

### Step 7: (S)—N—((R)-1-(4-carbamimidoylthiophen-2-yl)ethyl)-7-((2′,5′-difluoro-[1,1′-biphenyl]-4-carbonyl)glycyl)-1,4-dioxa-7-azaspiro[4.4]nonane-6-carboxamide (Compound 166)

To a mixture of (S)-7-((2′,5′-difluoro-[1,1′-biphenyl]-4-carbonyl)glycyl)-1,4-dioxa-7-azaspiro [4.4]nonane-8-carboxylic acid (50 mg, 0.11 mmol) and (S)-5-(1-aminoethyl)thiophene-3-carboximidamide (23 mg, 0.13 mmol) in DMF (2 mL) was added DIPEA (72 mg, 0.56 mmol) and PyBOP (87 mg, 0.17 mmol) at 0° C. under N₂ atmosphere and the mixture was stirred at 25° C. for 2 hours. The reaction mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-5% MeOH in DCM) and further purified by prep-HPLC to give Compound 166 (2.0 mg, yield 3.0%) as a white solid. ¹H NMR (400 MHz, CD₃OD) δ 8.53 (s, 1H), 8.24 (d, J=11.7 Hz, 1H), 7.95 (d, J=8.3 Hz, 2H), 7.66 (d, J=8.3 Hz, 2H), 7.54 (d, J=9.3 Hz, 1H), 7.32-7.22 (m, 2H), 7.19-7.13 (m, 1H), 5.31 (dq, J=13.7, 6.8 Hz, 1H), 4.60-4.55 (m, 1H), 4.25-4.11 (m, 2H), 4.03-3.94 (m, 4H), 3.84-3.75 (m, 2H), 2.44 (dd, J=13.1, 9.0 Hz, 1H), 2.21 (dd, J=13.1, 6.5 Hz, 1H), 1.61 (t, J=18.6 Hz, 3H). LC/MS (ESI) m/z: 598 (M+H)⁺.

### Scheme 158: Synthesis of (S)—N—((S)-1-(4-carbamimidoylthiophen-2-yl)ethyl)-7-((5-phenylpicolinoyl)glycyl)-1,4-dioxa-7-azaspiro[4.4]nonane-8-carboxamide (Compound 167)

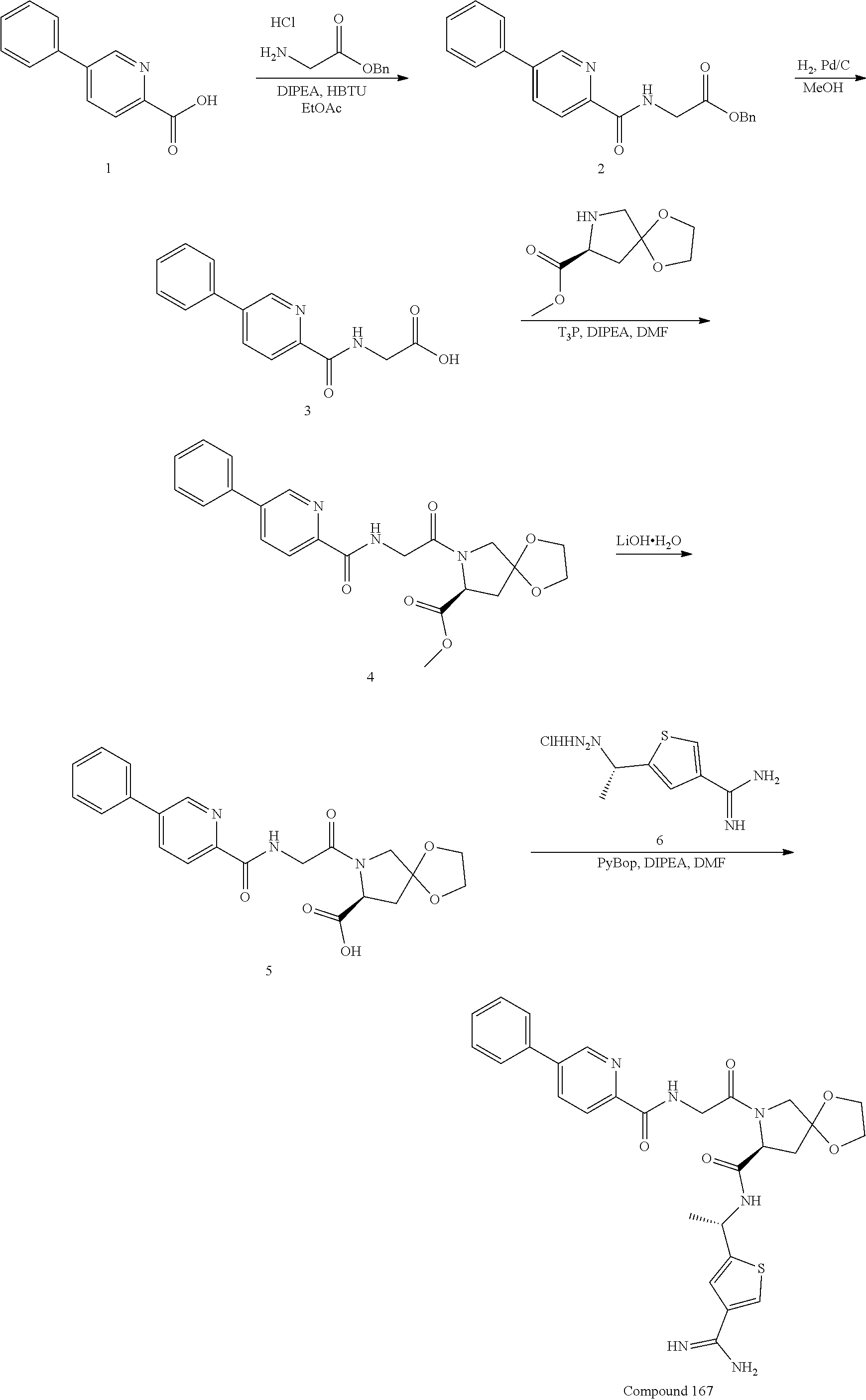

Compound 167 was prepared as a white solid from 5-phenylpicolinic acid, benzyl glycinate hydrochloride, methyl (S)-1,4-dioxa-7-azaspiro[4.4]nonane-8-carboxylate, and (S)-5-(1-aminoethyl)thiophene-3-carboximidamide hydrochloride based on the procedures of Scheme 12. ¹H NMR (400 MHz, CD₃OD) δ 8.92 (d, J=1.6 Hz, 1H), 8.55 (s, 1H), 8.20 (dd, J=10.4, 4.5 Hz, 2H), 8.14 (d, J=8.1 Hz, 1H), 7.73 (d, J=7.4 Hz, 2H), 7.57-7.49 (m, 3H), 7.46 (t, J=7.3 Hz, 1H), 5.37-5.23 (m, 1H), 4.61-4.53 (m, 1H), 4.26 (q, J=17.1 Hz, 2H), 4.03-3.94 (m, 4H), 3.77 (q, J=10.7 Hz, 2H), 2.44 (dd, J=13.0, 9.0 Hz, 1H), 2.21 (dd, J=13.1, 6.8 Hz, 1H), 1.61 (t, J=15.5 Hz, 3H). LCMS (ESI) (m/z): 563 (M+H)⁺.

### Scheme 159: Synthesis of (2S,4R)—N—((R)-1-(4-carbamimidoylthiophen-2-yl)ethyl)-1-((4-(difluoro(phenyl)methyl)benzoyl)glycyl)-4-(methylsulfonyl)pyrrolidine-2-carboxamide (Compound 168)

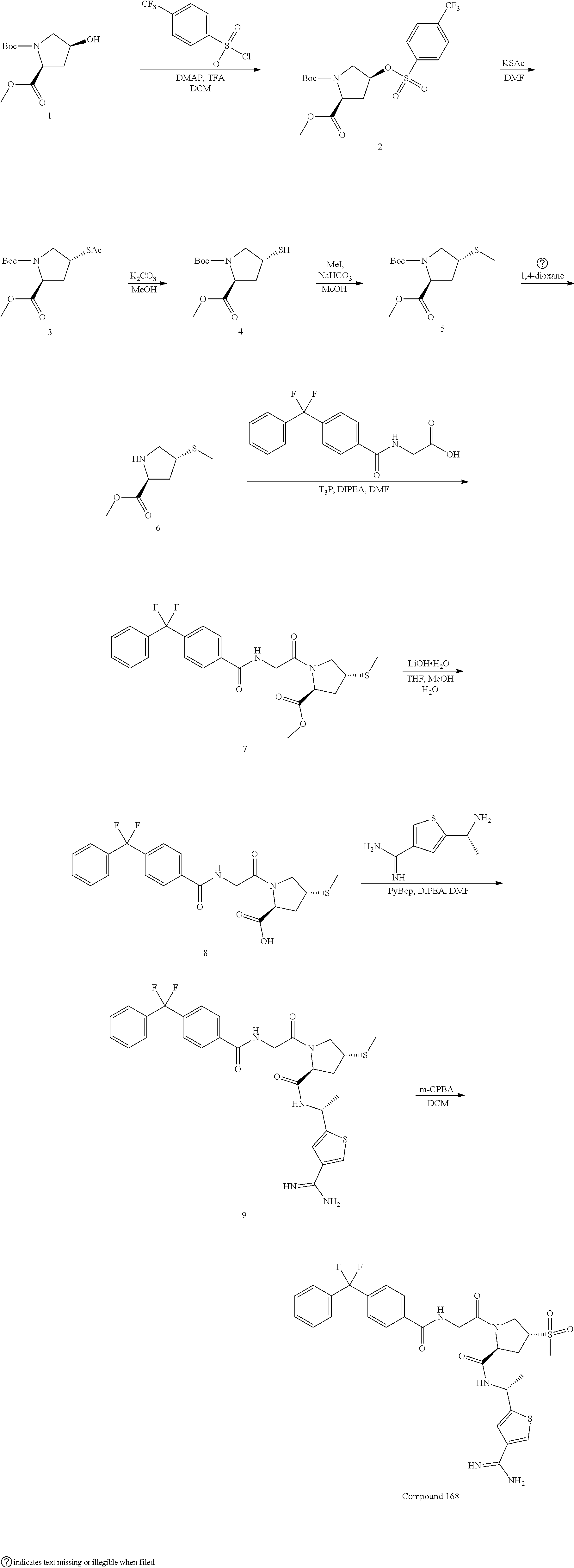

### Step 1: 1-(tert-butyl) 2-methyl (2S,4S)-4-(((4-(trifluoromethyl)phenyl)sulfonyl)oxy) pyrrolidine-1,2-dicarboxylate (2)

To a mixture of 1-(tert-butyl) 2-methyl (2S,4S)-4-hydroxypyrrolidine-1,2-dicarboxylate (10 g, 40.8 mmol) and 4-(trifluoromethyl)benzenesulfonylchloride (13.0 g, 53.0 mmol) in DCM (50 mL) was added TEA (5.0 g, 48.9 mmol) and DMAP (498 mg, 4.1 mmol) at 0° C. under N₂ atmosphere and the reaction mixture was stirred at room temperature overnight. The mixture was diluted with EtOAc, washed with 1 N aq·HCl solution and brine, dried over anhydrous Na₂SO₄, filtered, and concentrated under reduced pressure to dryness. The residue was purified by flash chromatography (silica gel, 0-5% MeOH in DCM) to give 1-(tert-butyl) 2-methyl (2S,4S)-4-(((4-(trifluoromethyl)phenyl)sulfonyl)oxy)pyrrolidine-1,2-dicarboxylate (17.1 g, yield 92.3%) as a white solid. LC/MS (ESI) m/z: 454 (M+H)⁺.

### Step 2: 1-(tert-butyl) 2-methyl (2S,4R)-4-(acetylthio)pyrrolidine-1,2-dicarboxylate (3)

To a solution of 1-(tert-butyl) 2-methyl (2S,4S)-4-(((4-(trifluoromethyl)phenyl)sulfonyl)oxy) pyrrolidine-1,2-dicarboxylate (16.5 g, 36.5 mmol) in DMF (100 mL) was added KSAc (8.3 g, 73.0 mmol) and the reaction mixture was stirred under N₂ atmosphere at 40° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give 1-(tert-butyl) 2-methyl (2R,4S)-4-(acetylthio)pyrrolidine-1,2-dicarboxylate (10.4 g, yield 93.4%) as a white solid. LC/MS (ESI) m/z: 304 (M+H)⁺.

### Step 3: 1-(tert-butyl) 2-methyl (2S,4R)-4-mercaptopyrrolidine-1,2-dicarboxylate (4)

To a solution of 1-(tert-butyl) 2-methyl (2S,4R)-4-(acetylthio)pyrrolidine-1,2-dicarboxylate (10.6 g, 35.1 mmol) in MeOH/DCM (110 mL, v/v=10/1) was added K₂CO₃ (4.9 g, 35.1 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give 1-(tert-butyl) 2-methyl (2S,4R)-4-mercaptopyrrolidine-1,2-dicarboxylate (7.2 g, yield 78.5%) as a white solid. LC/MS (ESI) m/z:262 (M+H)⁺.

### Step 4: 1-(tert-butyl) 2-methyl (2S,4R)-4-(methylthio)pyrrolidine-1,2-dicarboxylate (5)

To a solution of 1-(tert-butyl) 2-methyl (2S,4R)-4-mercaptopyrrolidine-1,2-dicarboxylate (7.2 g, 27.5 mmol) in MeOH (100 ml) was added MeI (40.3 g, 283.8 mmol) and NaHCO₃ (2.5 g, 30.3 mmol) under N₂ atmosphere and the reaction mixture was stirred at room temperature for 2 hours. The mixture was diluted with EtOAc and washed with water and brine, dried over anhydrous Na₂SO₄, filtered, and concentrated under reduced pressure to dryness. The residue was purified by flash chromatography (silica gel, 0-30% EtOAc in PE) to give 1-(tert-butyl) 2-methyl (2S,4R)-4-(methylthio)pyrrolidine-1,2-dicarboxylate (3.58 g, yield 47.2%) as a white solid. LC/MS (ESI) m/z: 276 (M+H)⁺.

### Step 5: methyl (2S,4R)-4-(methylthio)pyrrolidine-2-carboxylate hydrochloride (6)

To a solution of 1-(tert-butyl) 2-methyl (2S,4R)-4-(methylthio)pyrrolidine-1,2-dicarboxylate (500 mg, 1.82 mmol) in 1,4-dioxane (5 mL) was added HCl/1,4-dioxane (2 mL, 4M) under N₂ atmosphere and the mixture was stirred at room temperature overnight. The mixture was concentrated to dryness under reduced pressure to give methyl (2S,4R)-4-(methylthio)pyrrolidine-2-carboxylate hydrochloride (370 mg, yield 96%) as a white solid. LC/MS (ESI) m/z: 176 (M+H)⁺.

### Step 6: methyl (2S,4R)-1-((4-(difluoro(phenyl)methyl)benzoyl)glycyl)-4-(methylthio) pyrrolidine-2-carboxylate (7)

To a mixture of (4-(difluoro(phenyl)methyl)benzoyl)glycine (183 mg, 0.60 mmol) and methyl (2S,4R)-4-(methylthio)pyrrolidine-2-carboxylate hydrochloride (127 mg, 0.60 mmol) in DMF (5 mL) was added DIPEA (465 mg, 3.60 mmol) and T₃P (572 mg, 0.90 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-30% EtOAc in PE) to give methyl (2S,4R)-1-((4-(difluoro(phenyl)methyl)benzoyl)glycyl)-4-(methylthio)pyrrolidine-2-carboxylate (50 mg, yield 18.0%) as a white solid. LC/MS (ESI) m/z: 463 (M+H)⁺.

### Step 7: (2S,4R)-1-((4-(difluoro(phenyl)methyl)benzoyl)glycyl)-4-(methylthio)pyrrolidine-2-carboxylic acid (6)

To a solution of methyl (2S,4R)-1-((4-(difluoro(phenyl)methyl)benzoyl)glycyl)-4-(methylthio) pyrrolidine-2-carboxylate (50 mg, 0.11 mmol) in THF/MeOH/H₂O (3 mL, 4/1/1) was added LiOH (9 mg, 0.22 mmol) under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give (2S,4R)-1-((4-(difluoro(phenyl)methyl)benzoyl)glycyl)-4-(methylthio)pyrrolidine-2-carboxylic acid (45 mg, yield 92.8%) as a white solid. LC/MS (ESI) m/z: 449 (M+H)⁺.

### Step 8: (2S,4R)—N—((R)-1-(4-carbamimidoylthiophen-2-yl)ethyl)-1-((4-(difluoro(phenyl) methyl)benzoyl)glycyl)-4-(methylthio)pyrrolidine-2-carboxamide (9)

To a mixture of (2S,4R)-1-((4-(difluoro(phenyl)methyl)benzoyl)glycyl)-4-(methylthio) pyrrolidine-2-carboxylic acid (45 mg, 0.10 mmol) and (R)-5-(1-aminoethyl)thiophene-3-carboximidamide (34 mg, 0.20 mmol) in DMF (3 mL) was added DIPEA (78 mg, 0.60 mmol) and PyBop (78 mg, 0.15 mmol) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-5% MeOH in DCM) to give (2S,4R)—N—((R)-1-(4-carbamimidoylthiophen-2-yl)ethyl)-1-((4-(difluoro(phenyl)methyl)benzoyl)glycyl)-4-(methylthio)pyrrolidine-2-carboxamide (15 mg, yield 24.9%) as a white solid. LC/MS (ESI) m/z: 600 (M+H)⁺.

### Step 9: (2S,4R)—N—((R)-1-(4-carbamimidoylthiophen-2-yl)ethyl)-1-((4-(difluoro(phenyl) methyl)benzoyl)glycyl)-4-(methylsulfonyl)pyrrolidine-2-carboxamide (Compound 166)

To a solution of (2S,4R)—N—((R)-1-(4-carbamimidoylthiophen-2-yl)ethyl)-1-((4-(difluoro(phenyl) methyl)benzoyl)glycyl)-4-(methylthio)pyrrolidine-2-carboxamide (15 mg, 0.025 mmol) in DCM (3 mL) was added m-CPBA (13 mg, 0.075 mmol) under N₂ atmosphere and the reaction mixture was stirred at room temperature for 3 hours. The mixture was diluted with EtOAc, washed with water and brine, dried over anhydrous Na₂SO₄, filtered, and concentrated under reduced pressure to dryness. The residue was purified by prep·TLC (DCM:MeOH=5:1) and further purified by prep·HPLC to give Compound 168 (0.7 mg, yield 4.4%) as a white solid. ¹H NMR (400 MHz, CD₃OD) δ 8.55 (s, 1H), 8.23 (d, J=8.2 Hz, 1H), 7.92 (t, J=8.3 Hz, 2H), 7.62 (d, J=8.2 Hz, 2H), 7.50 (dt, J=10.8, 10.2 Hz, 6H), 5.26 (dd, J=14.2, 6.9 Hz, 1H), 4.70 (d, J=3.8 Hz, 1H), 4.28 (s, 2H), 4.21-4.11 (m, 3H), 3.09-3.03 (m, 3H), 2.83-2.70 (m, 1H), 2.43 (dd, J=13.5, 5.9 Hz, 1H), 1.61 (t, J=14.9 Hz, 3H). LC/MS (ESI) m/z: 632 (M+H)⁺.

### Scheme 160: Synthesis of (S)—N—((R)-1-(4-carbamimidoylthiophen-2-yl)ethyl)-7-((2′,4′-dimethyl-[1,1′-biphenyl]-4-carbonyl)glycyl)-1,4-dioxa-7-azaspiro[4.4]nonane-8-carboxamide (Compound 169)

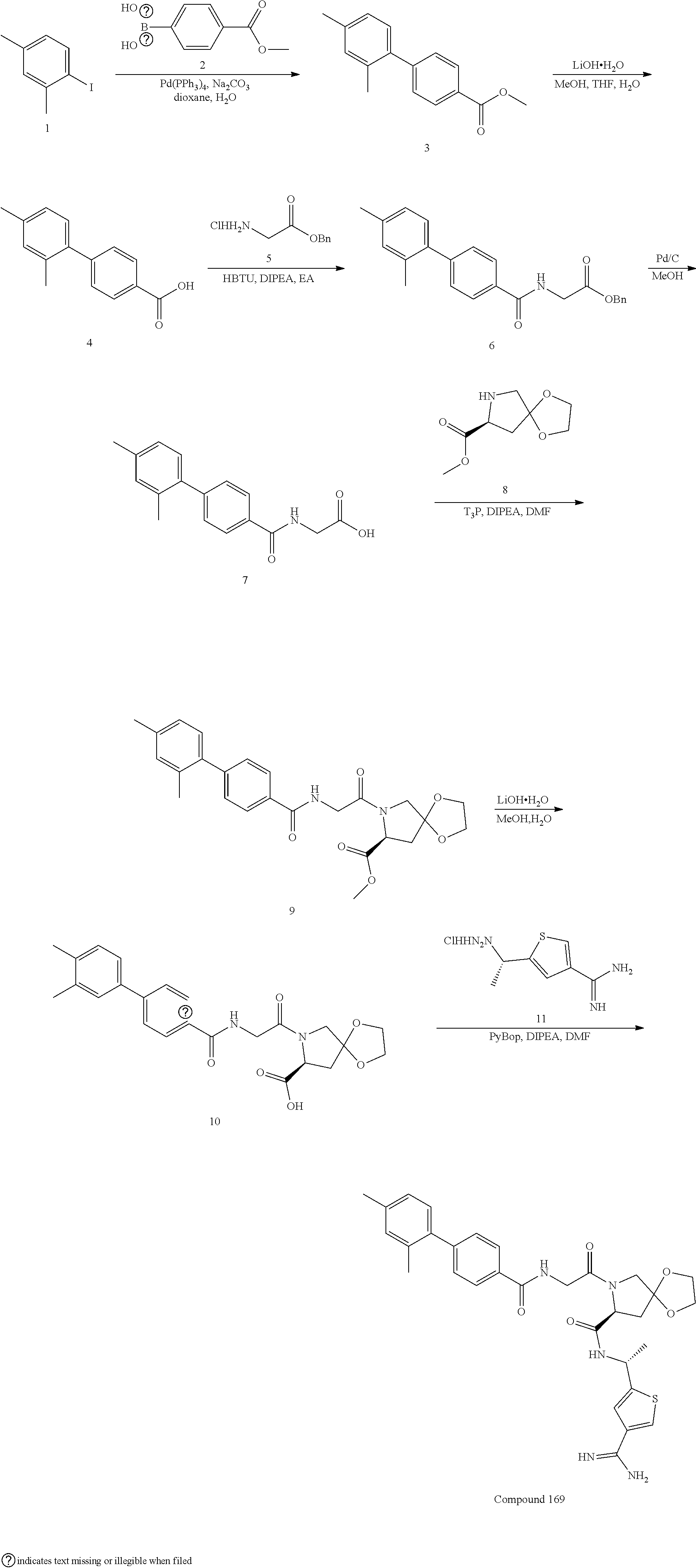

Compound 169 was prepared as a white solid from 1-iodo-2,4-dimethylbenzene, (4-(methoxycarbonyl)phenyl)boronic acid, benzyl glycinate hydrochloride, methyl (S)-1,4-dioxa-7-azaspiro[4.4]nonane-8-carboxylate, and (S)-5-(1-aminoethyl)thiophene-3-carboximidamide hydrochloride based on the procedures set forth in Scheme 150. ¹H NMR (400 MHz, CD₃OD) δ 8.56 (s, 1H), 8.25 (d, J=7.4 Hz, 1H), 7.91 (dd, J=11.2, 8.4 Hz, 2H), 7.54 (d, J=10.3 Hz, 1H), 7.43-7.37 (m, 2H), 7.13-7.05 (m, 3H), 5.31 (dt, J=20.4, 6.9 Hz, 1H), 4.58 (dd, J=8.6, 6.6 Hz, 1H), 4.26-4.06 (m, 2H), 4.05-3.74 (m, 5H), 3.58-3.32 (m, 1H), 2.44 (dd, J=13.1, 8.9 Hz, 1H), 2.35 (s, 3H), 2.28-2.06 (m, 4H), 1.67-1.54 (m, 3H). LCMS (ESI) (m/z): 590 (M+H)⁺.

### Scheme 161: Synthesis of (S)-7-((9H-carbazole-3-carbonyl)glycyl)-N—((R)-1-(4-carbamimidoylthiophen-2-yl)ethyl)-1,4-dioxa-7-azaspiro[4.4]nonane-8-carboxamide (Compound 170)

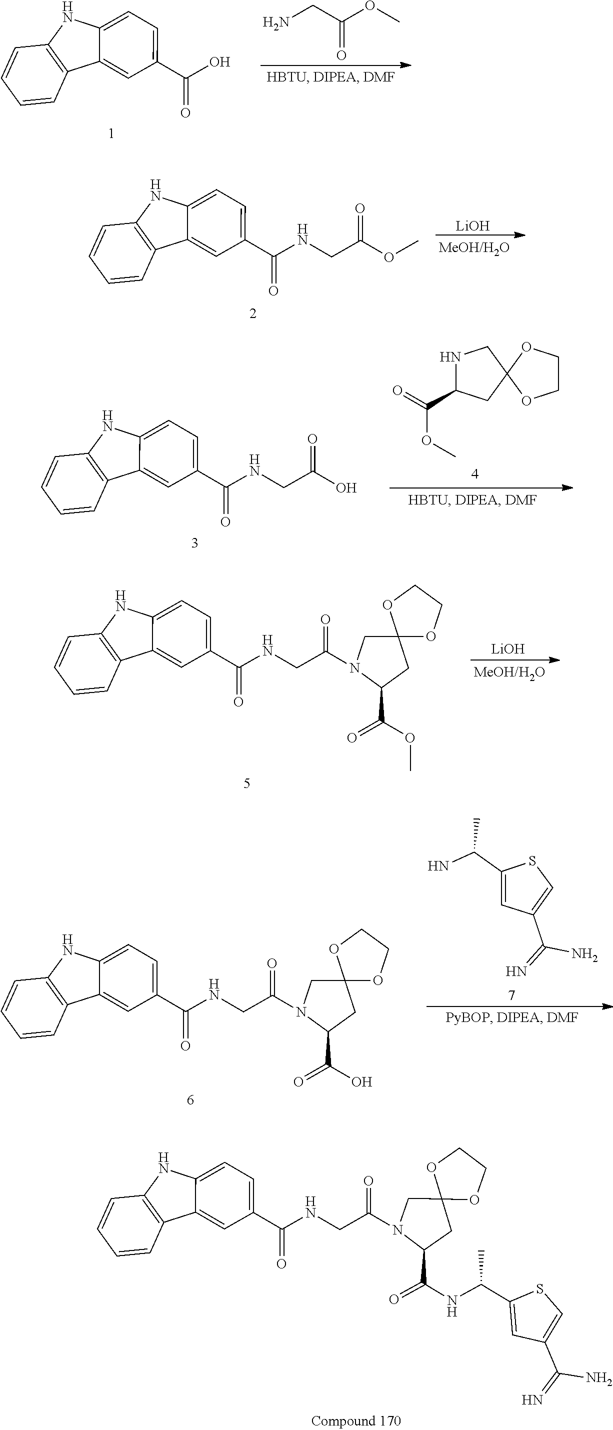

### Step 1: methyl (9H-carbazole-3-carbonyl)glycinate (2)

To a mixture of 9H-carbazole-3-carboxylic acid (250 mg, 1.2 mmol) and methyl glycinate hydrochloride (164 mg, 1.3 mmol) in DMF (3 mL) was added DIPEA (929 mg, 7.2 mmol) and HBTU (530 mg, 1.4 mmol) at 0° C. and the mixture was stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give methyl (9H-carbazole-3-carbonyl) glycinate (320 mg, yield 94.6%) as a white solid. LCMS (ESI) m/z: 283 (M+H)⁺.

### Step 2: (9H-carbazole-3-carbonyl)glycine (3)

To a solution of methyl (9H-carbazole-3-carbonyl)glycinate (320 mg, 1.1 mmol) in MeOH (3 mL) and water (1.5 mL) was added LiOH·H₂O (285 mg, 6.8 mmol) at 0° C. and the mixture was stirred at room temperature for 1 hour. The mixture was acidified with 1 N aq·HCl to pH˜3, extracted with EtOAc twice. The combined organic layers were washed with brine, dried over anhydrous Na₂SO₄, filtered, and concentrated under reduced pressure to dryness to give (9H-carbazole-3-carbonyl)glycine (300 mg, yield 99.1%) as a colorless oil, which was used directly in the next reaction. LCMS (ESI) (m/z): 269 (M+H)⁺.

### Step 3: methyl (S)-7-((9H-carbazole-3-carbonyl)glycyl)-1,4-dioxa-7-azaspiro[4.4]nonane-8-carboxylate (5)

To a mixture of (9H-carbazole-3-carbonyl)glycine (50 mg, 0.19 mmol) and methyl (S)-1,4-dioxa-7-azaspiro[4.4]nonane-8-carboxylate (32 mg, 0.17 mmol) in DMF (1 mL) was added DIPEA (147 mg, 1.1 mmol) and HBTU (87 mg, 0.23 mmol) at 0° C. and the mixture was stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give methyl(S)-7-((9H-carbazole-3-carbonyl)glycyl)-1,4-dioxa-7-azaspiro[4.4]nonane-8-carboxylate (70 mg, yield 84.3%) as a white solid. LCMS (ESI) m/z: 438 (M+H)⁺.

### Step 4: (S)-7-((9H-carbazole-3-carbonyl)glycyl)-1,4-dioxa-7-azaspiro[4.4]nonane-8-carboxylic acid (6)

To a solution of methyl (9H-carbazole-3-carbonyl)glycinate (50 mg, 0.11 mmol) in MeOH (0.6 mL) and water (0.4 mL) was added LiOH·H₂O (28 mg, 0.66 mmol) at 0° C. and the mixture was stirred at room temperature for 1 hour. The mixture was acidified with 1 N aq·HCl to pH ˜3 and extracted with EtOAc twice. The combined organic layers were washed with brine, dried over anhydrous Na₂SO₄, filtered, and concentrated under reduced pressure to dryness to give (S)-7-((9H-carbazole-3-carbonyl)glycyl)-1,4-dioxa-7-azaspiro[4.4]nonane-8-carboxylic acid (43 mg, yield 91.2%) as a colorless oil, which was used directly in the next reaction. LCMS (ESI) (m/z): 424 (M+H)⁺.

### Step 5: (S)-7-((9H-carbazole-3-carbonyl)glycyl)-N—((R)-1-(4-carbamimidoylthiophen-2-yl) ethyl)-1,4-dioxa-7-azaspiro[4.4]nonane-8-carboxamide (Compound 170)

To a mixture of (S)-7-((9H-carbazole-3-carbonyl)glycyl)-1,4-dioxa-7-azaspiro[4.4]nonane-8-carboxylic acid (43 mg, 0.10 mmol) and (R)-5-(1-aminoethyl)thiophene-3-carboximidamide (13 mg, 0.20 mmol) in DMF (1 mL) was added DIPEA (77 mg, 0.60 mmol) and PyBOP (52 mg, 0.10 mmol) at 0° C. and the mixture was stirred under N₂ atmosphere at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-8% MeOH in DCM) and further purified by prep-HPLC to give Compound 170 (14 mg, yield 24.4%) as a white solid. ¹H NMR (400 MHz, CD₃OD) δ 8.65 (s, 1H), 8.24 (d, J=16.3 Hz, 1H), 8.09 (d, J=8.1 Hz, 1H), 7.92 (d, J=8.1 Hz, 1H), 7.54 (s, 1H), 7.49 (d, J=8.4 Hz, 2H), 7.44 (d, J=7.1 Hz, 1H), 7.22 (t, J=7.4 Hz, 1H), 5.29 (d, J=7.0 Hz, 1H), 4.84-4.83 (m, 1H), 4.62-4.57 (m, 1H), 4.21 (s, 2H), 4.02-3.98 (m, 3H), 3.82 (t, J=6.7 Hz, 2H), 2.44 (dd, J=12.9, 8.9 Hz, 1H), 2.22 (dd, J=13.5, 6.3 Hz, 1H), 1.58 (d, J=7.0 Hz, 3H). LCMS (ESI) m/z: 575 (M+H)⁺.

### Scheme 162: Synthesis of (2S,4S)—N—((R)-1-(4-carbamimidoylthiophen-2-yl)ethyl)-1-((4-(4-fluorophenoxy)benzoyl)glycyl)-4-(thiazol-2-yl)pyrrolidine-2-carboxamide (Compound 171)

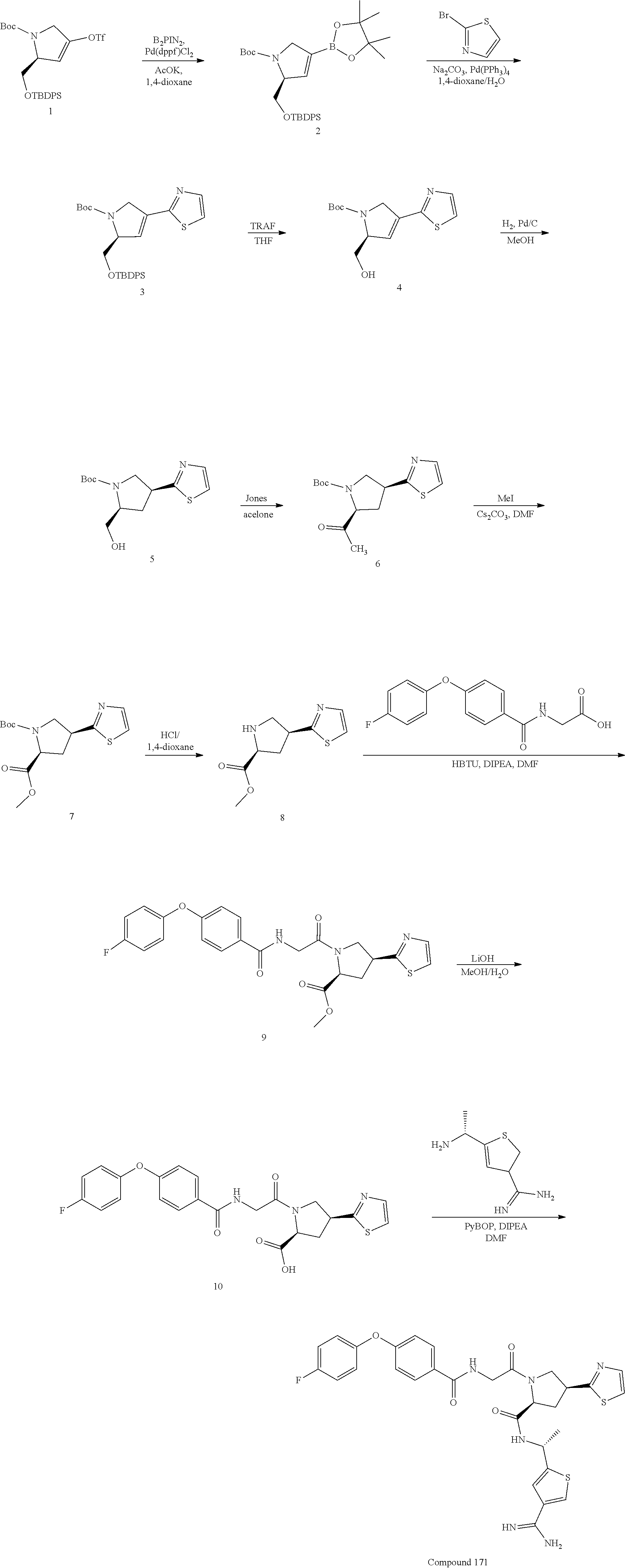

### Step 1: tert-butyl (S)-2-(((tert-butyldiphenylsilyl)oxy)methyl)-4-(4,4,5,5-tetramethyl-1,3,2-dioxaborolan-2-yl)-2,5-dihydro-1H-pyrrole-1-carboxylate (2)

To a mixture of tert-butyl (S)-2-(((tert-butyldiphenylsilyl)oxy)methyl)-4-(((trifluoromethyl) sulfonyl)oxy)-2,5-dihydro-1H-pyrrole-1-carboxylate (1.5 g, 2.6 mmol) and B₂Pin₂ (1.3 g, 5.1 mmol) in 1,4-dioxane (15 mL) was added Pd(dppf)Cl₂ (190 mg, 0.26 mmol) and AcOK (1.0 g, 10.2 mmol) under N₂ atmosphere at 0° C. The mixture was degassed under N₂ atmosphere for three times and stirred at 80° C. overnight. The reaction mixture was directly used in next step without work-up.

### Step 2: tert-butyl (S)-2-(((tert-butyldiphenylsilyl)oxy)methyl)-4-(thiazol-2-yl)-2,5-dihydro-1H-pyrrole-1-carboxylate (3)

To the mixture of previous step was added 2-bromothiazole (820 mg, 5.0 mmol), Pd(PPh₃)₄ (289 mg, 0.25 mmol), Na₂CO₃ (1.0 g, 10 mmol) and water (10 mL). The new mixture was degassed under N₂ atmosphere for three times and stirred at 85° C. for 3 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-15% EtOAc in PE) to give tert-butyl (S)-2-(((tert-butyldiphenylsilyl)oxy)methyl)-4-(thiazol-2-yl)-2,5-dihydro-1H-pyrrole-1-carboxylate (800 mg, yield 60.1% via two steps) as a yellow oil. LCMS (ESI) m/z: 521 (M+H)⁺.

### Step 3: tert-butyl (S)-2-(hydroxymethyl)-4-(thiazol-2-yl)-2,5-dihydro-1H-pyrrole-1-carboxylate (4)

To a solution of tert-butyl (S)-2-(((tert-butyldiphenylsilyl)oxy)methyl)-4-(thiazol-2-yl)-2,5-dihydro-1H-pyrrole-1-carboxylate (800 mg, 1.5 mmol) in THF (5 mL) was added TBAF (3 mL, 3 mmol, 1 M in THF) at 0° C. and the mixture was stirred at room temperature for 1 hour. The mixture was diluted with EtOAc, washed with saturated aq·NH₄Cl solution and brine, dried over anhydrous Na₂SO₄, filtered, and concentrated under reduced pressure to dryness. The residue was purified by flash chromatography (silica gel, 0-30% EtOAc in PE) to give tert-butyl (S)-2-(hydroxymethyl)-4-(thiazol-2-yl)-2,5-dihydro-1H-pyrrole-1-carboxylate (400 mg, yield 97.8%) as a yellow oil. LCMS (ESI) m/z: 283 (M+H)⁺.

### Step 4: tert-butyl (2S,4S)-2-(hydroxymethyl)-4-(thiazol-2-yl)pyrrolidine-1-carboxylate (5)

To a solution of tert-butyl (S)-2-(hydroxymethyl)-4-(thiazol-2-yl)-2,5-dihydro-1H-pyrrole-1-carboxylate (400 mg, 1.4 mmol) in MeOH (5 mL) was added Pd/C (80 mg, 10% wt.), the mixture was degassed under N₂ atmosphere for three times and stirred under a H₂ balloon at room temperature overnight. The mixture was filtered, and the filtrate was concentrated under reduced pressure to dryness to give tert-butyl (2S,4S)-2-(hydroxymethyl)-4-(thiazol-2-yl)pyrrolidine-1-carboxylate (320 mg, yield 79.3%) as a colorless oil, which was used directly in the next reaction. LCMS (ESI) (m/z): 285 (M+H)⁺.

### Step 5: (2S,4S)-1-(tert-butoxycarbonyl)-4-(thiazol-2-yl)pyrrolidine-2-carboxylic acid (6)

To a mixture of tert-butyl (S)-2-(hydroxymethyl)-4-(thiazol-2-yl)-2,5-dihydro-1H-pyrrole-1-carboxylate (320 mg, 1.1 mmol) in acetone (4.0 mL) was added Jones oxidant (0.7 mL) at 0° C. and the mixture was stirred under N₂ atmosphere at room temperature for 20 minutes. The mixture was quenched with saturated aq·Na₂S₂O₃ solution and extracted with EtOAc twice. The combined organic layers were washed with brine, dried over anhydrous Na₂SO₄, filtered, and concentrated under reduced pressure to dryness to give the (2S,4S)-1-(tert-butoxycarbonyl)-4-(thiazol-2-yl)pyrrolidine-2-carboxylic acid (290 mg, yield 88.5%) as a yellow oil, which was used directly in the next reaction. LCMS (ESI) m/z: 299 (M+H)⁺.

### Step 6: 1-(tert-butyl) 2-methyl (2S,4S)-4-(thiazol-2-yl)pyrrolidine-1,2-dicarboxylate (7)

To a mixture of (2S,4S)-1-(tert-butoxycarbonyl)-4-(thiazol-2-yl)pyrrolidine-2-carboxylic acid (290 mg, 0.97 mmol) and Cs₂CO₃ (1.3 g, 3.9 mmol) in DMF (4 mL) was added MeI (824 mg, 5.8 mmol) under N₂ atmosphere at room temperature and the reaction mixture was stirred overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give 1-(tert-butyl) 2-methyl (2S,4S)-4-(thiazol-2-yl)pyrrolidine-1,2-dicarboxylate (110 mg, yield 36.3%) as a yellow oil. LCMS (ESI) m/z: 313 (M+H)⁺.

### Step 7: methyl (2S,4S)-4-(thiazol-2-yl)pyrrolidine-2-carboxylate hydrochloride (8)

A mixture of 1-(tert-butyl) 2-methyl (2S,4S)-4-(thiazol-2-yl)pyrrolidine-1,2-dicarboxylate (110 mg, 0.35 mmol) in HCl/1,4-dioxane (2.0 mL) was stirred at room temperature for 30 minutes. The reaction mixture was concentrated under reduced pressure to dryness, co-evaporated with DCM, dried under vacuum to give methyl (2S,4S)-4-(thiazol-2-yl)pyrrolidine-2-carboxylate hydrochloride (85 mg, yield 97.6% yield) as a yellow solid, which was used directly in the next reaction without further purification. LCMS (ESI) m/z: 213 (M+H)⁺.

### Step 8: methyl (2S,4S)-1-((4-(4-fluorophenoxy)benzoyl)glycyl)-4-(thiazol-2-yl)pyrrolidine-2-carboxylate (9)

To a mixture of methyl (2S,4S)-4-(thiazol-2-yl)pyrrolidine-2-carboxylate (33 mg, 0.16 mmol) and (4-(4-fluorophenoxy)benzoyl)glycine (115 mg, 0.42 mmol) in DMF (1.0 mL) was added DIPEA (124 mg, 0.96 mmol) and HBTU (72 mg, 0.19 mmol) at 0° C. and the mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give methyl(2S,4S)-1-((4-(4-fluorophenoxy)benzoyl)glycyl)-4-(thiazol-2-yl)pyrrolidine-2-carboxylate (27 mg, yield 34.9%) as a white solid. LCMS (ESI) (m/z): 484 (M+H)⁺.

### Step 9: (2S,4S)-1-((4-(4-fluorophenoxy)benzoyl)glycyl)-4-(thiazol-2-yl)pyrrolidine-2-carboxylic acid (10)

To a solution of methyl (2S,4S)-1-((4-(4-fluorophenoxy)benzoyl)glycyl)-4-(thiazol-2-yl) pyrrolidine-2-carboxylate (27 mg, 0.13 mmol) in MeOH (1.0 mL) and water (0.5 mL) was added LiOH (14 mg, 0.34 mmol) at 0° C. and the mixture was stirred at room temperature for 1 hour. The mixture was acidified with 1 N aq·HCl to pH-3 and extracted with EtOAc twice. The combined organic layers were washed with brine, dried over anhydrous Na₂SO₄, filtered, and concentrated under reduced pressure to dryness to give (2S,4S)-1-((4-(4-fluorophenoxy) benzoyl)glycyl)-4-(thiazol-2-yl)pyrrolidine-2-carboxylic acid (25 mg, yield 94.6%) as a colorless oil, which was used directly in the next reaction. LCMS (ESI) (m/z): 470 (M+H)⁺.

### Step 10: (2S,4S)—N—((R)-1-(4-carbamimidoylthiophen-2-yl)ethyl)-1-((4-(4-fluorophenoxy) benzoyl)glycyl)-4-(thiazol-2-yl)pyrrolidine-2-carboxamide (Compound 171)

To a mixture of (2S,4S)-1-((4-(4-fluorophenoxy)benzoyl)glycyl)-4-(thiazol-2-yl)pyrrolidine-2-carboxylic acid (25 mg, 0.053 mmol) and (R)-5-(1-aminoethyl)thiophene-3-carboximidamide (19 mg, 0.11 mmol) in DMF (1.0 mL) was added DIPEA (41 mg, 0.32 mmol) and PyBOP (30 mg, 0.058 mmol) at 0° C. and the mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) and further purified by prep·HPLC to give Compound 171 (4.8 mg, yield 14.6%) as a white solid. ¹H NMR (400 MHz, CD₃OD) δ 8.12 (s, 1H), 7.85 (d, J=8.7 Hz, 2H), 7.74 (d, J=3.3 Hz, 1H), 7.55-7.49 (m, 2H), 7.16 (t, J=8.6 Hz, 2H), 7.09 (dd, J=9.0, 4.6 Hz, 2H), 6.99 (d, J=8.7 Hz, 2H), 5.26 (d, J=6.9 Hz, 1H), 4.57 (t, J=8.5 Hz, 1H), 4.35 (dd, J=16.8, 6.2 Hz, 2H), 4.14 (d, J=16.5 Hz, 1H), 4.01 (s, 1H), 3.87 (t, J=9.8 Hz, 1H), 2.89-2.81 (m, 1H), 2.32-2.25 (m, 1H), 1.58 (d, J=7.0 Hz, 3H). LCMS (ESI) (m/z): 621 (M+H)⁺.

### Scheme 163: Synthesis of (2S,4S)—N—((R)-1-(4-carbamimidoylthiophen-2-yl)ethyl)-1-((9,9-difluoro-9H-fluorene-3-carbonyl)glycyl)-4-(thiazol-2-yl)pyrrolidine-2-carboxamide (Compound 172)

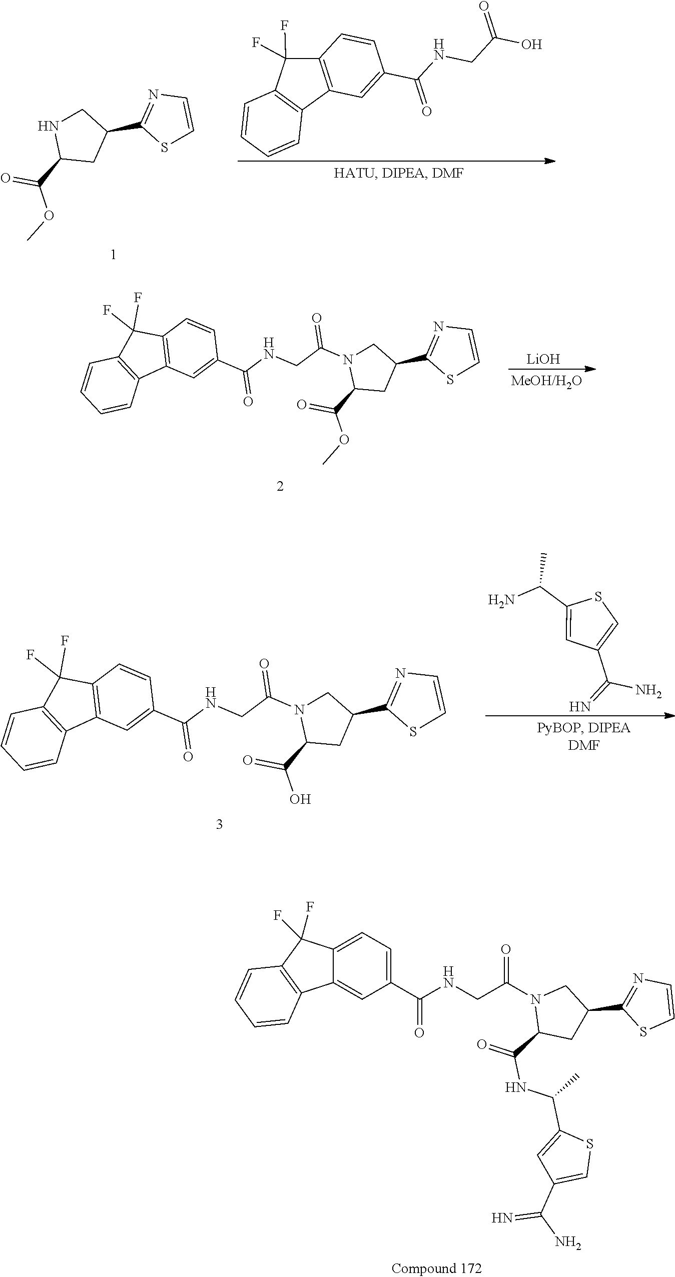

Compound 172 was prepared as a white solid from methyl (S)-4-(thiazol-2-yl)pyrrolidine-2-carboxylate,(9,9-difluoro-9H-fluorene-3-carbonyl)glycine, and (R)-5-(1-aminoethyl)thiophene-3-carboximidamide based on the procedures set forth in Scheme 3. ¹H NMR (400 MHz, CD₃OD) δ 8.12 (s, 1H), 7.85 (d, J=8.7 Hz, 2H), 7.74 (d, J=3.3 Hz, 1H), 7.55-7.49 (m, 2H), 7.16 (t, J=8.6 Hz, 2H), 7.09 (dd, J=9.0, 4.6 Hz, 2H), 6.99 (d, J=8.7 Hz, 2H), 5.26 (d, J=6.9 Hz, 1H), 4.57 (t, J=8.5 Hz, 1H), 4.35 (dd, J=16.8, 6.2 Hz, 2H), 4.14 (d, J=16.5 Hz, 1H), 4.01 (s, 1H), 3.87 (t, J=9.8 Hz, 1H), 2.89-2.81 (m, 1H), 2.32-2.25 (m, 1H), 1.58 (d, J=7.0 Hz, 3H). LCMS (ESI) (m/z): 635 (M+H)⁺.

### Scheme 164: Synthesis of (S)—N—((R)-1-(4-carbamimidoylthiophen-2-yl)ethyl)-7-((4-(3,5-dimethylpyridin-2-yl)benzoyl)glycyl)-1,4-dioxa-7-azaspiro[4.4]nonane-8-carboxamide (Compound 173)

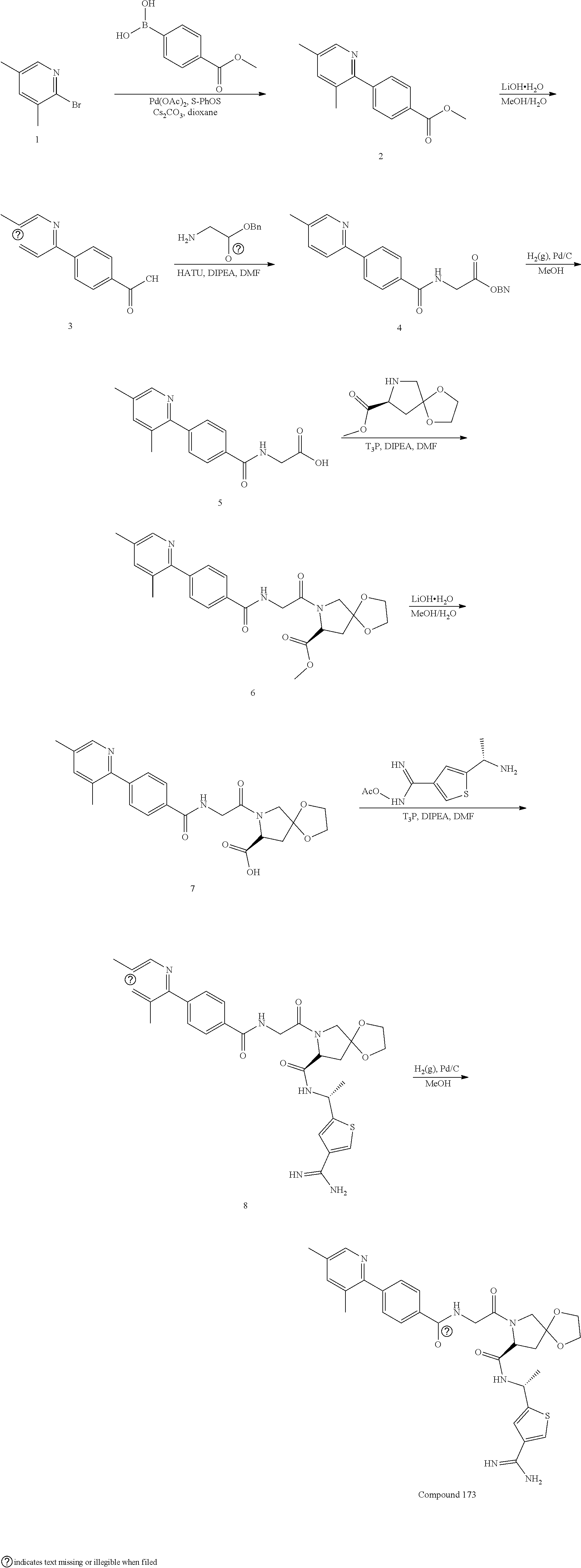

### Step 1: methyl 4-(3,5-dimethylpyridin-2-yl)benzoate (2)

To a mixture of 2-bromo-3,5-dimethylpyridine (1.0 g, 5.38 mmol) and (4-(methoxycarbonyl)phenyl)boronic acid (1.26 g, 6.45 mmol) in 1,4-dioxane (13 mL) was added Cs₂CO₃ (5.26 g, 16.14 mmol), S-Phos (110 mg, 0.27 mmol) and Pd(OAc)₂ (60 mg, 0.27 mmol) under N₂ atmosphere, the reaction mixture was degassed under N₂ atmosphere for three times and stirred at 110° C. overnight. The mixture was diluted with EtOAc twice, washed with water and brine, dried over anhydrous Na₂SO₄, filtered, and concentrated under reduced pressure to dryness. The residue was purified by flash chromatography (silica gel, 0-20% EtOAc in PE) to give methyl 4-(3,5-dimethylpyridin-2-yl)benzoate (1.2 g, yield 92.3%) as a white solid. LC/MS (ESI) m/z: 242 (M+H)⁺.

### Step 2: 4-(3,5-dimethylpyridin-2-yl)benzoic acid (3)

To a solution of methyl 4-(3,5-dimethylpyridin-2-yl)benzoate (1.3 g, 5.39 mmol) in MeOH (4 mL), THF (4 mL) and water (3 mL) was added LiOH·H₂O (680 mg, 16.18 mmol) at 0° C. and the mixture was stirred at room temperature for 2 hours. The mixture was acidified with 1 N aq·HCl to pH˜3 and extracted with EtOAc twice. The combined organic layers were washed with brine, dried over anhydrous Na₂SO₄, filtered, and concentrated to dryness to give 4-(3,5-dimethylpyridin-2-yl)benzoic acid (1.05 g, yield 86.1%) as a white solid, which was used directly in the next reaction. LC/MS (ESI) (m/z): 228 (M+H)⁺.

### Step 3: benzyl (4-(3,5-dimethylpyridin-2-yl)benzoyl)glycinate (4)

To a mixture of 4-(3,5-dimethylpyridin-2-yl)benzoic acid (1.22 g, 5.37 mmol) and benzyl glycinate (1.63 g, 8.06 mmol) in DMF (20 mL) was added HATU (2.45 g, 6.44 mmol) and DIPEA (5.3 mL, 32.22 mmol) under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give benzyl (4-(3,5-dimethylpyridin-2-yl)benzoyl)glycinate (1.8 g, yield 90.0%) as a colorless oil. LC/MS (ESI) m/z: 375 (M+H)⁺.

### Step 4: (4-(3,5-dimethylpyridin-2-yl)benzoyl)glycine (5)

To a solution of benzyl (4-(3,5-dimethylpyridin-2-yl)benzoyl)glycinate (1.8 g, 4.81 mmol) in MeOH (20 mL) was added Pd/C (30 mg, 10% wt.) at room temperature, the mixture was degassed under N₂ atmosphere for three times and stirred under a H₂ balloon at room temperature for 2 hours. The mixture was filtered, and the filtrate was concentrated under reduced pressure to dryness to give (4-(3,5-dimethylpyridin-2-yl)benzoyl)glycine (1.2 g, yield 88.2%) as a white solid, which was used directly in the next step. LC/MS (ESI) (m/z): 285 (M+H)⁺.

### Step 5: methyl (S)-7-((4-(3,5-dimethylpyridin-2-yl)benzoyl)glycyl)-1,4-dioxa-7-azaspiro[4.4]nonane-8-carboxylate (6)

To a mixture of (4-(3,5-dimethylpyridin-2-yl)benzoyl)glycine (90 mg, 0.32 mmol) and methyl (S)-1,4-dioxa-7-azaspiro[4.4]nonane-8-carboxylate (60 mg, 0.32 mmol) in DMF (3 mL) was added DIPEA (0.31 mL, 1.92 mmol) and T₃P (611 mg, 0.96 mmol, 50% wt. in EtOAc) under N₂ atmosphere and the mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-10% MeOH in DCM) to give methyl (S)-7-((4-(3,5-dimethylpyridin-2-yl)benzoyl)glycyl)-1,4-dioxa-7-azaspiro[4.4]nonane-8-carboxylate (113 mg, yield 79.0%) as a yellow oil. LC/MS (ESI) m/z: 454 (M+H)⁺.

### Step 6: (S)-7-((4-(3,5-dimethylpyridin-2-yl)benzoyl)glycyl)-1,4-dioxa-7-azaspiro[4.4]nonane-8-carboxylic acid (7)

To a solution of methyl (S)-7-((4-(3,5-dimethylpyridin-2-yl)benzoyl)glycyl)-1,4-dioxa-7-azaspiro[4.4]nonane-8-carboxylate (130 mg, 0.29 mmol) in MeOH (3 mL) and water (1 mL) was added LiOH·H₂O (18 mg, 0.44 mmol) at 0° C. and the mixture was stirred at room temperature for 2 hours. The mixture was acidified with 1 N aq·HCl to pH˜4 and extracted with EtOAc twice. The combined organic layers were washed with brine, dried over anhydrous Na₂SO₄, filtered, and concentrated under reduced pressure to give (S)-7-((4-(3,5-dimethylpyridin-2-yl)benzoyl)glycyl)-1,4-dioxa-7-azaspiro[4.4]nonane-8-carboxylic acid (50 mg, yield 39.7%) as a white solid, which was used directly in the next reaction. LC/MS (ESI) (m/z): 440 (M+H)⁺.

### Step 7: (S)—N—((R)-1-(4-(N-acetoxycarbamimidoyl)thiophen-2-yl)ethyl)-7-((4-(3,5-dimethylpyridin-2-yl)benzoyl)glycyl)-1,4-dioxa-7-azaspiro[4.4]nonane-8-carboxamide (8)

To a mixture of (S)-7-((4-(3,5-dimethylpyridin-2-yl)benzoyl)glycyl)-1,4-dioxa-7-azaspiro [4.4]nonane-8-carboxylic acid (50 mg, 0.11 mmol) and (S)—N-acetoxy-5-(1-aminoethyl) thiophene-3-carboximidamide (52 mg, 0.22 mmol) in DMF (2 mL) was added DIPEA (88 mg, 0.66 mmol) and T₃P (217 mg, 0.33 mmol, 50% wt. in EtOAc) at 0° C. under N₂ atmosphere and the mixture was stirred at 25° C. for 1 hour. The reaction mixture was diluted with EtOAc washed with saturated aq·NaHCO₃ solution and brine, dried over anhydrous Na₂SO₄, filtered, and concentrated under reduced pressure to dryness. The residue was purified by prep·TLC (DCM:MeOH=10:1) to give (S)—N—((R)-1-(4-(N-acetoxycarbamimidoyl)thiophen-2-yl)ethyl)-7-((4-(3,5-dimethylpyridin-2-yl)benzoyl)glycyl)-1,4-dioxa-7-azaspiro[4.4]nonane-8-carboxamide (20 mg, yield 27.0%) as a white solid. LC/MS (ESI) m/z: 649 (M+H)⁺.

### Step 8: (S)—N—((R)-1-(4-carbamimidoylthiophen-2-yl)ethyl)-7-((4-(3,5-dimethylpyridin-2-yl) benzoyl)glycyl)-1,4-dioxa-7-azaspiro[4.4]nonane-8-carboxamide (Compound 173)

To a solution of (S)—N—((R)-1-(4-(N-acetoxycarbamimidoyl)thiophen-2-yl)ethyl)-7-((4-(3,5-dimethylpyridin-2-yl)benzoyl)glycyl)-1,4-dioxa-7-azaspiro[4.4]nonane-8-carboxamide (20 mg, 0.03 mmol) in MeOH (3 mL) was added Pd/C (5 mg, 10% wt.) at room temperature, the mixture was degassed under N₂ atmosphere for three times and stirred under a H₂ balloon at room temperature for 2 hours. The mixture was filtered, and the filtrate was concentrated under reduced pressure to dryness. The residue was purified by prep-HPLC to give Compound 173 (3.0 mg, yield 18.2%) as a white solid. ¹H NMR (400 MHz, CD₃OD) δ 8.55 (s, 1H), 8.26 (d, J=23.5 Hz, 2H), 7.96 (d, J=8.2 Hz, 2H), 7.64 (s, 1H), 7.60-7.54 (m, 3H), 5.28 (d, J=7.0 Hz, 1H), 4.58 (t, J=7.6 Hz, 1H), 4.26-4.11 (m, 2H), 4.04-3.95 (m, 4H), 3.80 (q, J=11.0 Hz, 2H), 2.47-2.40 (m, 1H), 2.39 (s, 3H), 2.31 (s, 3H), 2.22 (dd, J=13.3, 6.7 Hz, 1H), 1.63 (dd, J=37.5, 6.6 Hz, 3H). LC/MS (ESI) (m/z): 591 (M+H)⁺.

### Scheme 165: Synthesis of (S)—N—((R)-1-(4-carbamimidoylthiophen-2-yl)ethyl)-7-((4′-chloro-2′-fluoro-[1,1′-biphenyl]-4-carbonyl)glycyl)-1,4-dioxa-7-azaspiro[4.4]nonane-8-carboxamide (Compound 174)

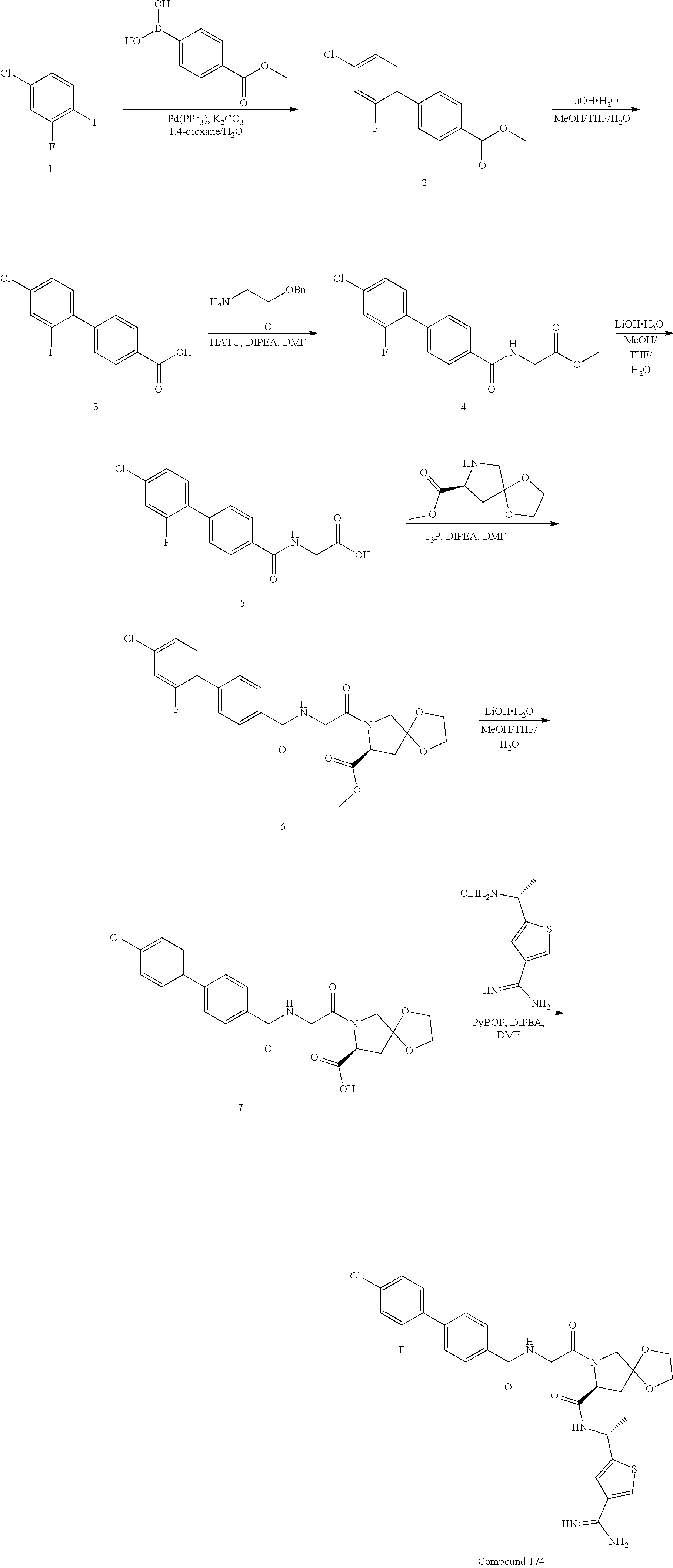

### Step 1: methyl 4′-chloro-2′-fluoro-[1,1′-biphenyl]-4-carboxylate (2)

To a mixture of 4-chloro-2-fluoro-1-iodobenzene (1.0 g, 3.9 mmol) and (4-(methoxycarbonyl)phenyl)boronic acid (912 mg, 5.07 mmol) in 1,4-dioxane (10 mL) and water (3 mL) was added K₂CO₃ (1.6 g, 11.7 mmol) and Pd(PPh₃)₄ (225 mg, 0.20 mmol) at 25° C. under N₂ atmosphere and the reaction mixture was degassed under N₂ atmosphere for three times and stirred at 100° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give methyl 4′-chloro-2′-fluoro-[1,1′-biphenyl]-4-carboxylate (550 mg, yield 53.4%) as a colorless oil.

### Step 2: 4′-chloro-2′-fluoro-[1,1′-biphenyl]-4-carboxylic acid (3)

To a solution of methyl 4′-chloro-2′-fluoro-[1,1′-biphenyl]-4-carboxylate (550 mg, 2.08 mmol) in MeOH (6 mL), THF (3 mL) and water (3 mL) was added LiOH·H₂O (175 mg, 4.17 mmol) at 0° C. and the mixture was stirred at room temperature for 16 hours. The mixture was acidified with 1 N aq·HCl to pH-3 and extracted with EtOAc twice. The combined organic layers were washed with brine, dried over anhydrous Na₂SO₄, filtered, and concentrated under reduced pressure to give 4′-chloro-2′-fluoro-[1,1′-biphenyl]-4-carboxylic acid (500 mg, yield 95.8%) as a white solid, which was used directly in the next reaction. LC/MS (ESI) (m/z): 251 (M−H)⁻.

### Step 3: methyl (4′-chloro-2′-fluoro-[1,1′-biphenyl]-4-carbonyl)glycinate (4)

To a mixture of 4′-chloro-2′-fluoro-[1,1′-biphenyl]-4-carboxylic acid (500 mg, 1.99 mmol) and methyl glycinate hydrochloride (325 mg, 2.59 mmol) in DMF (5 mL) was added HBTU (1.13 g, 2.99 mmol) and DIPEA (1.29 g, 9.95 mmol) and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography flash chromatography (silica gel, 0-30% EtOAc in PE) to give methyl (4′-chloro-2′-fluoro-[1,1′-biphenyl]-4-carbonyl)glycinate (500 mg, yield 78.1%) as a colorless oil. LC/MS (ESI) m/z: 322 (M+H)⁺.

### Step 4: (4′-chloro-2′-fluoro-[1,1′-biphenyl]-4-carbonyl)glycine (5)

To a solution of methyl (4′-chloro-2′-fluoro-[1,1′-biphenyl]-4-carbonyl)glycinate (150 mg, 0.47 mmol) in MeOH (1 mL), THF (0.5 mL) and water (0.5 mL) was added LiOH·H₂O (39 mg, 0.94 mmol) at 0° C. and the mixture was stirred at room temperature for 2 hours. The mixture was acidified with 1 N aq·HCl to pH˜3 and extracted with EtOAc twice. The combined organic layers were washed with brine, dried over anhydrous Na₂SO₄, filtered, and concentrated under reduced pressure to give (4′-chloro-2′-fluoro-[1,1′-biphenyl]-4-carbonyl)glycine (130 mg, yield 89.8%) as a white solid, which was used directly in the next reaction. LC/MS (ESI) (m/z): 308 (M+H)⁺.

### Step 5: methyl (S)-7-((4′-chloro-2′-fluoro-[1,1′-biphenyl]-4-carbonyl)glycyl)-1,4-dioxa-7-azaspiro[4.4]nonane-8-carboxylate (6)

To a mixture of (4′-chloro-2′-fluoro-[1,1′-biphenyl]-4-carbonyl)glycine (82 mg, 0.27 mmol) and methyl (S)-1,4-dioxa-7-azaspiro[4.4]nonane-8-carboxylate (50 mg, 0.27 mmol) in DMF (2 mL) was added DIPEA (103 mg, 0.80 mmol) and T₃P (254 mg, 0.40 mmol, 50% wt. in EtOAc) under N₂ atmosphere and the mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-5% MeCOH in DCM) to give methyl (S)-7-((4′-chloro-2′-fluoro-[1,1′-biphenyl]-4-carbonyl)glycyl)-1,4-dioxa-7-azaspiro[4.4]nonane-8-carboxylate (83 mg, yield 65.9%) as a colorless oil. LC/MS (ESI) m/z: 477 (M+H)⁺.

### Step 6: (S)-7-((4′-chloro-2′-fluoro-[1,1′-biphenyl]-4-carbonyl)glycyl)-1,4-dioxa-7-azaspiro[4.4]nonane-6-carboxylic acid (7)

To a solution of methyl (S)-7-((4′-chloro-2′-fluoro-[1,1′-biphenyl]-4-carbonyl)glycyl)-1,4-dioxa-7-azaspiro[4.4]nonane-8-carboxylate (83 mg, 0.17 mmol) in MeOH (1 mL), THF (0.5 mL) and water (0.5 mL) was added LiOH·H₂O (15 mg, 0.35 mmol) at 0° C. and the mixture was stirred at room temperature for 2 hours. The mixture was acidified with 1 N aq·HCl to pH-4, extracted with EtOAc twice. The combined organic layers were washed with brine, dried over anhydrous Na₂SO₄, filtered, and concentrated under reduced pressure to give (S)-7-((4′-chloro-2′-fluoro-[1,1′-biphenyl]-4-carbonyl)glycyl)-1,4-dioxa-7-azaspiro[4.4]nonane-8-carboxylic acid (77 mg, yield 97.8%) as a white solid, which was used directly in the next reaction. LC/MS (ESI) (m/z): 463 (M+H)⁺.

### Step 7: (S)—N—((R)-1-(4-carbamimidoylthiophen-2-yl)ethyl)-7-((4′-chloro-2′-fluoro-[1,1′-biphenyl]-4-carbonyl)glycyl)-1,4-dioxa-7-azaspiro[4.4]nonane-8-carboxamide (Compound 174)

To a mixture of (S)-7-((4′-chloro-2′-fluoro-[1,1′-biphenyl]-4-carbonyl)glycyl)-1,4-dioxa-7-azaspiro[4.4]nonane-8-carboxylic acid (39 mg, 0.084 mmol) and (R)-5-(1-aminoethyl)thiophene-3-carboximidamide hydrochloride (21 mg, 0.10 mmol) in DMF (1 mL) was added DIPEA (55 mg, 0.47 mmol) and PyBOP (48 mg, 0.093 mmol) at 0° C. under N₂ atmosphere and the mixture was stirred at 250° C. for 2 hours. The reaction mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-5% MeOH in DCM) and further purified by prep-HPLC to give Compound 174 (6.8 mg, yield 13.2%) as a white solid. ¹H NMR (400 MHz, CD₃OD) δ 8.55 (s, 1H), 8.17 (s, 1H), 7.95 (d, J=8.2 Hz, 2H), 7.65 (d, J=7.4 Hz, 2H), 7.56-7.51 (m, 2H), 7.36-7.32 (m, 2H), 5.28 (q, J=6.8 Hz, 1H), 4.62-4.54 (m, 1H), 4.20-4.11 (m, 2H), 4.01-3.95 (m, 4H), 3.80 (q, J=10.6 Hz, 2H), 2.43 (dd, J=12.9, 8.9 Hz, 1H), 2.21 (dd, J=13.1, 6.6 Hz, 1H), 1.62 (dd, J=36.1, 6.9 Hz, 3H). LC/MS (ESI) m/z: 614 (M+H)⁺.

### Scheme 166: Synthesis of (S)—N—((R)-1-(4-carbamimidoylthiophen-2-yl)ethyl)-7-((2,4′-difluoro-[1,1′-biphenyl]-4-carbonyl)glycyl)-1,4-dioxa-7-azaspiro[4.4]nonane-8-carboxamide (Compound 175)

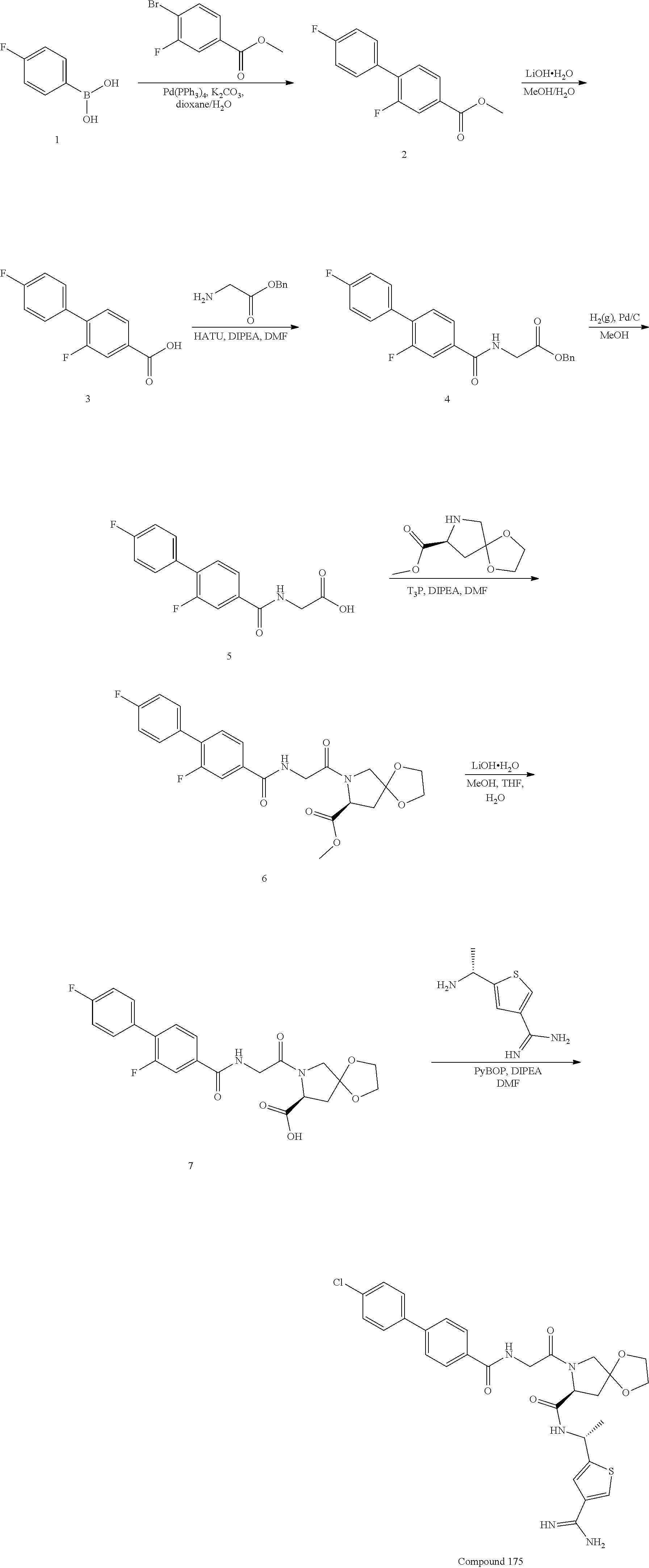

Compound 175 was prepared as a white solid from methyl 4-bromo-3-fluorobenzoate, (4-fluorophenyl)boronic acid, benzyl glycinate, methyl (S)-7-((2,4′-difluoro-[1,1′-biphenyl]-4-carbonyl)glycyl)-1,4-dioxa-7-azaspiro[4.4]nonane-8-carboxylate, and (R)-5-(1-aminoethyl)thiophene-3-carboximidamide based on the procedures set forth in Scheme 150. ¹H NMR (400 MHz, CD₃OD) δ 8.55 (s, 1H), 8.24 (t, J=4.8 Hz, 1H), 7.76 (d, J=8.1 Hz, 1H), 7.69 (d, J=11.5 Hz, 1H), 7.64-7.58 (m, 3H), 7.53 (d, J=7.5 Hz, 1H), 7.22 (t, J=8.8 Hz, 2H), 5.29 (q, J=6.9 Hz, 1H), 4.62-4.53 (m, 1H), 4.24-4.12 (m, 2H), 4.03-3.95 (m, 4H), 3.79 (q, J=10.6 Hz, 2H), 2.44 (dd, J=13.0, 8.9 Hz, 1H), 2.21 (dd, J=13.0, 6.5 Hz, 1H), 1.61 (t, J=18.5 Hz, 3H). LC/MS (ESI) m/z: 598 (M+H)⁺.

### Scheme 167: Synthesis of (S)—N—((R)-1-(4-carbamimidoylthiophen-2-yl)ethyl)-7-((2′-chloro-4′-fluoro-[1,1′-biphenyl]-4-carbonyl)glycyl)-1,4-dioxa-7-azaspiro[4.4]nonane-8-carboxamide (Compound 176)

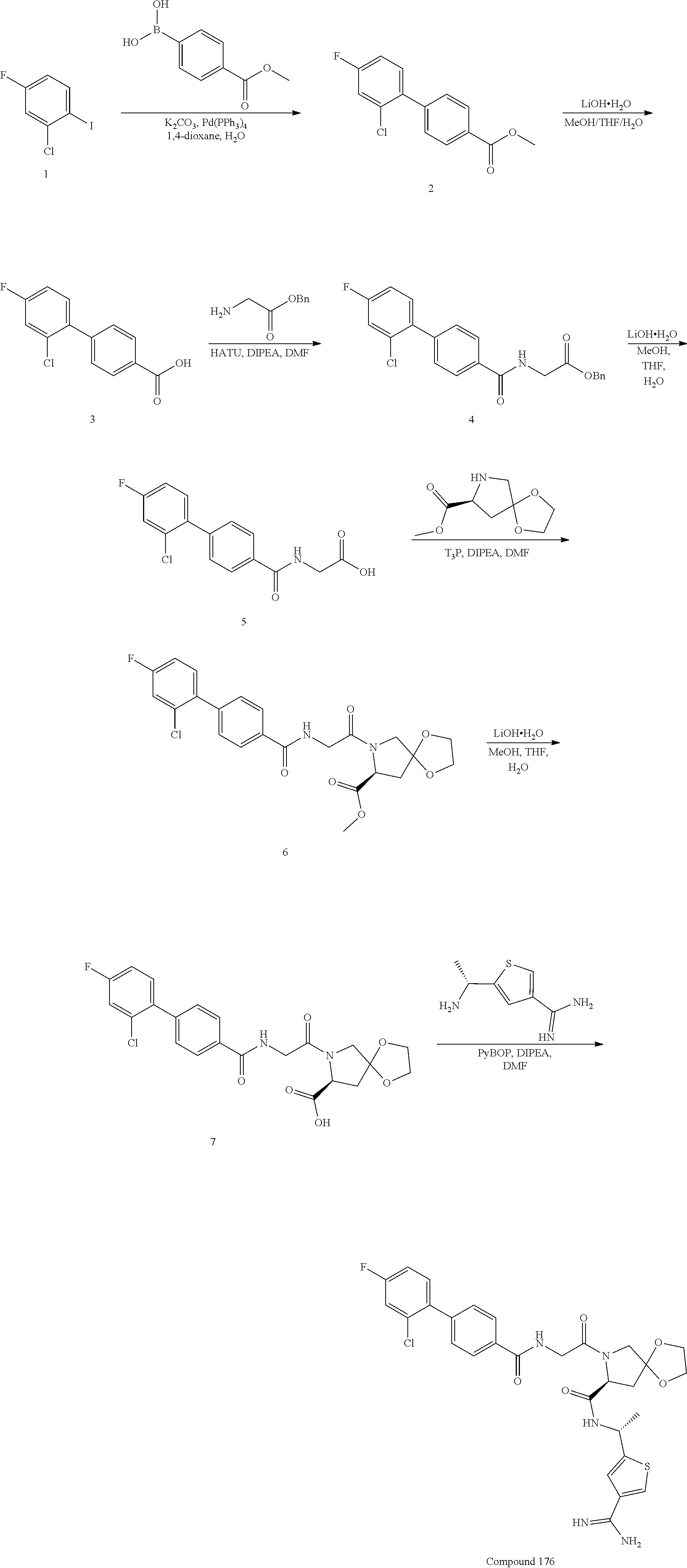

Compound 176 was prepared as a white solid from 2-chloro-4-fluoro-7-iodobenzene, (4-(methoxycarbonyl)phenyl)boronic acid, benzyl glycinate, methyl (S)-1,4-dioxa-7-azaspiro[4.4]nonane-8-carboxylate, and (R)-5-(1-aminoethyl) thiophene-3-carboximidamide based on the procedures set forth in Scheme 165. ¹H NMR (400 MHz, CD₃OD) δ 8.54 (s, 1H), 8.24 (d, J=10.0 Hz, 1H), 7.93 (d, J=8.1 Hz, 2H), 7.55-7.50 (m, 3H), 7.46-7.40 (m, 1H), 7.36 (dd, J=8.9, 2.2 Hz, 1H), 7.19 (t, J=7.1 Hz, 1H), 5.29 (d, J=7.1 Hz, 1H), 4.56 (s, 1H), 4.19 (s, 2H), 3.99 (dd, J=12.5, 8.1 Hz, 4H), 3.79 (t, J=8.3 Hz, 2H), 2.47-2.41 (m, 1H), 2.21 (d, J=6.5 Hz, 1H), 1.58 (d, J=6.8 Hz, 3H). LC/MS (ESI) m/z: 614 (M+H)⁺.

### Scheme 168: Synthesis of (S)—N—((R)-1-(4-carbamimidoylthiophen-2-yl)ethyl)-7-((9,9-difluoro-5-methyl-9H-fluorene-3-carbonyl)glycyl)-1,4-dioxa-7-azaspiro[4.4]nonane-8-carboxamide (Compound 177)

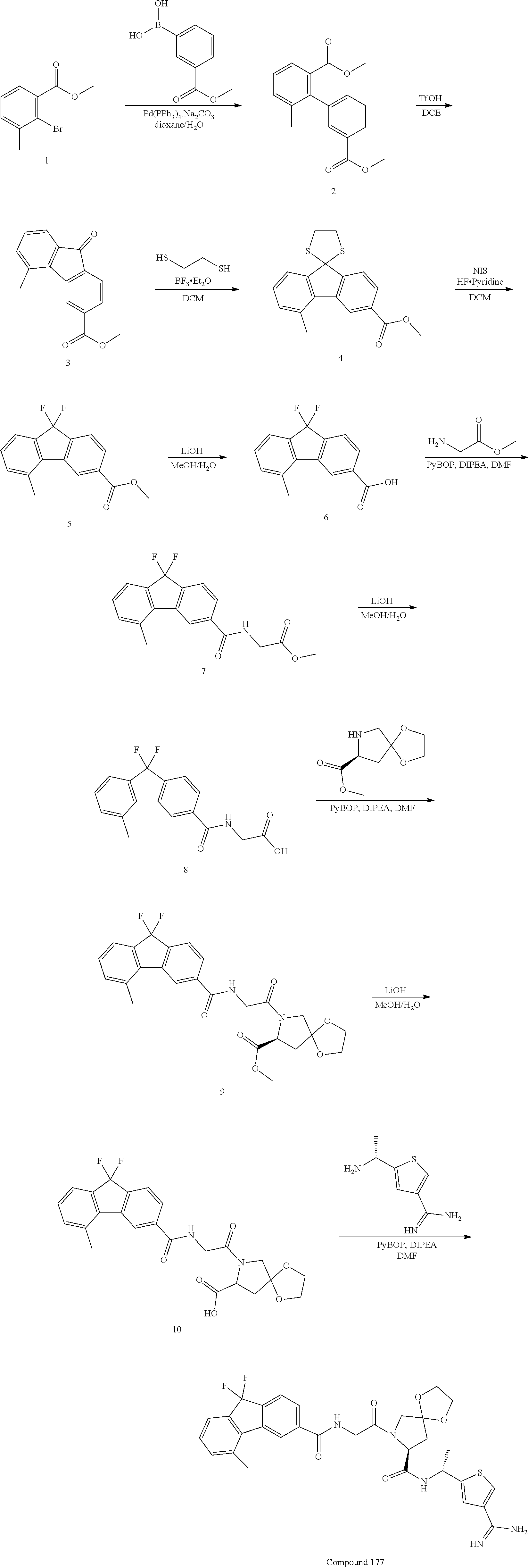

### Step 1: dimethyl 6-methyl-[1,1′-biphenyl]-2,3′-dicarboxylate (2)

To a mixture of methyl 2-bromo-3-methylbenzoate (1.0 g, 4.4 mmol) and (3-(methoxycarbonyl)phenyl)boronic acid (1.6 g, 8.8 mmol) in 1,4-dioxane (10 mL) and water (6 mL) was added Na₂CO₃ (1.0 g, 11 mmol) and Pd(PPh₃)₄ (300 mg, 0.26 mmol) at 25° C. under N₂ atmosphere, degassed under N₂ atmosphere for three times and stirred at 90° C. for 4 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give dimethyl 6-methyl-[1,1′-biphenyl]-2,3′-dicarboxylate (1.2 g, yield 95.5%) as a yellow oil.

### Step 2: methyl 5-methyl-9-oxo-9H-fluorene-3-carboxylate (3)

To a solution of dimethyl 6-methyl-[1,1′-biphenyl]-2,3′-dicarboxylate (1.2 g, 4.2 mmol) in DCE (15 mL) was added TfOH (7.9 g, 53 mmol) at 25° C. under N₂ atmosphere and stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-5% EtOAc in PE) to give methyl 5-methyl-9-oxo-9H-fluorene-3-carboxylate (500 mg, yield 47.6%) as a yellow oil.

### Step 3: methyl 5-methylspiro[fluorene-9,2′-[1,3]dithiolane]-3-carboxylate (4)

To a solution of methyl 5-methyl-9-oxo-9H-fluorene-3-carboxylate (150 mg, 0.60 mmol) in DCM (1.5 mL) was added ethane-1,2-dithiol (282 mg, 3.0 mmol) and BF₃·Et₂O (0.3 mL, 98%) dropwisely at 0° C. under N₂ atmosphere and stirred for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-3% EtOAc in PE) to give methyl 5-methylspiro[fluorene-9,2′-[1,3]dithiolane]-3-carboxylate (160 mg, yield 81.3%) as a colorless oil.

### Step 4: methyl 9,9-difluoro-5-methyl-9H-fluorene-3-carboxylate (5)

To a solution of NIS (765 mg, 3.4 mmol) in DCM (2 mL) was added HF/Pyridine (0.4 mL, 65%) dropwisely at 0° C. under N₂ atmosphere and stirred for 15 minutes. The mixture was added to a solution of methyl 5-methylspiro[fluorene-9,2′-[1,3]dithiolane]-3-carboxylate (160 mg, 0.49 mmol) in DCM (1.0 mL) dropwisely and the reaction mixture was stirred overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give methyl 9,9-difluoro-5-methyl-9H-fluorene-3-carboxylate (70 mg, yield 52.1%) as a yellow solid.

### Step 5: 9,9-difluoro-5-methyl-9H-fluorene-3-carboxylic acid (6)

To a solution of methyl 9,9-difluoro-5-methyl-9H-fluorene-3-carboxylate (70 mg, 0.26 mmol) in MeOH/H₂O (2 mL, 1/1) was added LiOH·H₂O (66 mg, 1.6 mmol) and stirred at room temperature for 2 hours. The mixture was acidified with 1 N aq·HCl to pH˜3 and extracted with EtOAc twice. The combined organic layers were washed with brine, dried over anhydrous Na₂SO₄, filtered, and concentrated to dryness under reduced pressure to give 9,9-difluoro-5-methyl-9H-fluorene-3-carboxylic acid (60 mg, yield 88.8%) as a white solid. LC/MS (ESI) m/z: 261 (M+H)⁺.

### Step 6: methyl (9,9-difluoro-5-methyl-9H-fluorene-3-carbonyl)glycinate (7)

To a mixture of 9,9-difluoro-5-methyl-9H-fluorene-3-carboxylic acid (60 mg, 0.23 mmol) and methyl glycinate (57 mg, 0.46 mmol) in DMF (1 mL) was added PyBop (146 mg, 0.28 mmol) and DIPEA (178 mg, 1.4 mmol) under N₂ atmosphere at room temperature and the reaction mixture was stirred for 2 hours. The reaction mixture was diluted with water and extracted with EtOAc twice. The combined organic layers were washed with brine, dried over anhydrous Na₂SO₄, filtered, and concentrated to dryness under reduced pressure. The residue was purified by column chromatography (silica gel, 0-40% EtOAc in PE) to give methyl (9,9-difluoro-5-methyl-9H-fluorene-3-carbonyl)glycinate (50 mg, yield 65.7%) as a yellow solid. LCMS (ESI) m/z: 332 (M+H)⁺.

### Step 7: (9,9-difluoro-5-methyl-9H-fluorene-3-carbonyl)glycine (6)

To a solution of methyl (9,9-difluoro-5-methyl-9H-fluorene-3-carbonyl)glycinate (50 mg, 0.15 mmol) in MeOH/H₂O (1.5 mL, 1/1) was added LiOH·H₂O (38 mg, 0.91 mmol) and the reaction was stirred at room temperature for 2 hours. The mixture was acidified with 1 N aq·HCl to pH˜3 and extracted with EtOAc twice. The combined organic layers were washed with brine, dried over anhydrous Na₂SO₄, filtered, and concentrated to dryness under reduced pressure to give (9,9-difluoro-5-methyl-9H-fluorene-3-carbonyl)glycine (46 mg, yield 96.7%) as a white solid. LC/MS (ESI) m/z: 318 (M+H)⁺.

### Step 8: methyl (S)-7-((9,9-difluoro-5-methyl-9H-fluorene-3-carbonyl)glycyl)-1,4-dioxa-7-azaspiro[4.4]nonane-6-carboxylate (9)

To a mixture of (9,9-difluoro-5-methyl-9H-fluorene-3-carbonyl)glycine (46 mg, 0.14 mmol) and methyl (S)-1,4-dioxa-7-azaspiro[4.4]nonane-8-carboxylate (27 mg, 0.14 mmol) in DMF (0.6 mL) was added PyBOP (90 mg, 0.17 mmol) and DIPEA (112 mg, 0.87 mmol) under N₂ atmosphere and the reaction mixture was stirred for 2 hours. The reaction mixture was diluted with water and extracted with EtOAc twice. The combined organic layers were washed with brine, dried over anhydrous Na₂SO₄, filtered, and concentrated to dryness under reduced pressure. The residue was purified by column chromatography (silica gel, 0-50% EtOAc in PE) to give methyl (S)-7-((9,9-difluoro-5-methyl-9H-fluorene-3-carbonyl)glycyl)-1,4-dioxa-7-azaspiro[4.4]nonane-8-carboxylate (53 mg, yield 75.2%) as a yellow solid. LCMS (ESI) m/z: 487 (M+H)⁺.

### Step 9: (S)-7-((9,9-difluoro-5-methyl-9H-fluorene-3-carbonyl)glycyl)-1,4-dioxa-7-azaspiro [4.4]nonane-8-carboxylic acid (10)

To a solution of methyl (S)-7-((9,9-difluoro-5-methyl-9H-fluorene-3-carbonyl)glycyl)-1,4-dioxa-7-azaspiro[4.4]nonane-8-carboxylate (53 mg, 0.11 mmol) in MeOH/H₂O (1.5 mL, 1/1) was added LiOH·H₂O (28 mg, 0.66 mmol) and stirred at room temperature for 2 hours. The mixture was acidified with 1 N aq·HCl to pH-3 and extracted with EtOAc twice. The combined organic layers were washed with brine, dried over anhydrous Na₂SO₄, filtered, and concentrated to dryness under reduced pressure to give (S)-7-((9,9-difluoro-5-methyl-9H-fluorene-3-carbonyl)glycyl)-1,4-dioxa-7-azaspiro[4.4]nonane-8-carboxylic acid (48 mg, yield 92.4%) as a white solid. LC/MS (ESI) m/z: 473 (M+H)⁺.

### Step 10: (S)—N—((R)-1-(4-carbamimidoylthiophen-2-yl)ethyl)-7-((9,9-difluoro-5-methyl-9H-fluorene-3-carbonyl)glycyl)-1,4-dioxa-7-azaspiro[4.4]nonane-6-carboxamide (Compound 177)

To a mixture of (S)-7-((9,9-difluoro-5-methyl-9H-fluorene-3-carbonyl)glycyl)-1,4-dioxa-7-azaspiro[4.4]nonane-8-carboxylic acid (48 mg, 0.10 mmol) and (R)-5-(1-aminoethyl)thiophene-3-carboximidamide (34 mg, 0.20 mmol) in DMF (0.6 mL) was added PyBOP (62 mg, 0.12 mmol) and DIPEA (77 mg, 0.60 mmol) under N₂ atmosphere and the reaction mixture was stirred for 2 hours. The reaction mixture was diluted with water and extracted with EtOAc twice. The combined organic layers were washed with brine, dried over anhydrous Na₂SO₄, filtered, and concentrated to dryness under reduced pressure. The residue was purified by column chromatography (silica gel, 0-10% MeOH in DCM) to give Compound 177 (12 mg, yield 19.3%) as a white solid. ¹H NMR (400 MHz, CD₃OD) δ 8.54 (s, 1H), 8.22 (d, J=5.4 Hz, 2H), 7.87 (d, J=7.4 Hz, 1H), 7.73 (d, J=7.8 Hz, 1H), 7.50 (d, J=10.4 Hz, 2H), 7.35 (d, J=7.4 Hz, 2H), 5.28 (d, J=7.2 Hz, 1H), 4.58 (s, 3H), 4.20 (d, J=7.0 Hz, 2H), 4.00 (d, J=4.4 Hz, 2H), 3.81 (d, J=5.4 Hz, 2H), 2.69 (d, J=9.4 Hz, 3H), 2.44 (dd, J=13.0, 8.9 Hz, 1H), 2.21 (dd, J=13.2, 6.5 Hz, 1H), 1.61 (t, J=18.7 Hz, 3H). LCMS (ESI) m/z: 624 (M+H)⁺.

### Scheme 169: Synthesis of (2S,4R)—N—((R)-1-(4-carbamimidoylthiophen-2-yl)ethyl)-1-((9,9-dimethyl-9H-fluorene-3-carbonyl)glycyl)-4-(methylsulfonyl)pyrrolidine-2-carboxamide (Compound 178)

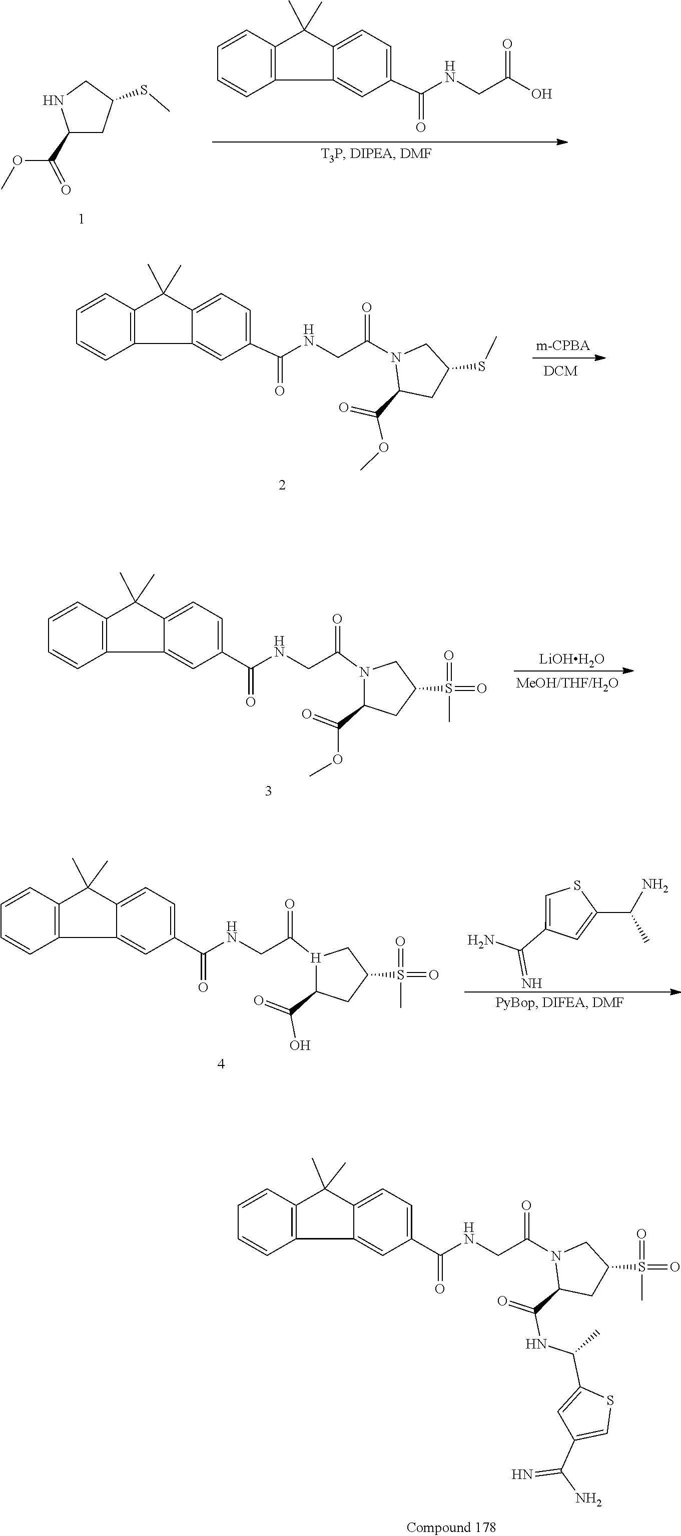

### Step 1: methyl (2S,4R)-1-((9,9-dimethyl-9H-fluorene-3-carbonyl)glycyl)-4-(methylthio) pyrrolidine-2-carboxylate (2)

To a mixture of (9,9-dimethyl-9H-fluorene-3-carbonyl)glycine (522 mg, 1.77 mmol) and methyl (2S,4R)-4-(methylthio)pyrrolidine-2-carboxylate (310 mg, 1.77 mmol) in DMF (10 mL) was added DIPEA (1.4 g, 10.61 mmol) and T₃P (1.7 g, 2.65 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-25% EtOAc in PE) to give methyl (2S,4R)-1-((9,9-dimethyl-9H-fluorene-3-carbonyl)glycyl)-4-(methylthio)pyrrolidine-2-carboxylate (630 mg, yield 78.7%) as a white solid. LC/MS (ESI) m/z: 453 (M+H)⁺.

### Step 2: methyl (2S,4R)-1-((9,9-dimethyl-9H-fluorene-3-carbonyl)glycyl)-4-(methylsulfonyl) pyrrolidine-2-carboxylate (3)

To a solution of methyl (2S,4R)-1-((9,9-dimethyl-9H-fluorene-3-carbonyl)glycyl)-4-(methylthio)pyrrolidine-2-carboxylate (630 mg, 1.39 mmol) in DCM (10 mL) was added m-CPBA (480 mg, 2.78 mmol) under N₂ atmosphere and the reaction mixture was stirred at room temperature for 3 hours. The mixture was diluted with EtOAc and washed with water and brine, dried over anhydrous Na₂SO₄, filtered, and concentrated under reduced pressure to dryness. The residue was purified by flash chromatography (silica gel, 0-5% MeOH in DCM) to give methyl (2S,4R)-1-((9,9-dimethyl-9H-fluorene-3-carbonyl)glycyl)-4-(methylsulfonyl)pyrrolidine-2-carboxylate (660 mg, yield 97.8%) as a white solid. LC/MS (ESI) m/z: 485 (M+H)⁺.

### Step 3: (2S,4R)-1-((9,9-dimethyl-9H-fluorene-3-carbonyl)glycyl)-4-(methylsulfonyl) pyrrolidine-2-carboxylic acid (4)

To a solution of methyl (2S,4R)-1-((9,9-dimethyl-9H-fluorene-3-carbonyl)glycyl)-4-(methylsulfonyl)pyrrolidine-2-carboxylate (660 mg, 1.36 mmol) in THF/MeOH/H₂O (9 mL,4/1/1) was added LiOH·H₂O (114 mg, 2.72 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give (2S,4R)-1-((9,9-dimethyl-9H-fluorene-3-carbonyl)glycyl)-4-(methylsulfonyl)pyrrolidine-2-carboxylic acid (630 mg, yield 98.3%) as a white solid. LC/MS (ESI) m/z:471 (M+H)⁺.

### Step 4: (2S,4R)—N—((R)-1-(4-carbamimidoylthiophen-2-yl)ethyl)-1-((9,9-dimethyl-9H-fluorene-3-carbonyl)glycyl)-4-(methylsulfonyl)pyrrolidine-2-carboxamide (Compound 178)

To a mixture of (2S,4R)-1-((9,9-dimethyl-9H-fluorene-3-carbonyl)glycyl)-4-(methylsulfonyl) pyrrolidine-2-carboxylic acid (80 mg, 0.17 mmol) and (R)-5-(1-aminoethyl)thiophene-3-carboximidamide (35 mg, 0.20 mmol) in DMF (3 mL) was added DIPEA (132 mg, 1.02 mmol) and PyBop (133 mg, 0.26 mmol) under N₂ atmosphere and the reaction mixture was stirred at room temperature for 2 hours. The mixture was diluted with EtOAc and washed with water and brine, dried over anhydrous Na₂SO₄, filtered, and concentrated under reduced pressure to dryness. The residue was purified by prep-TLC (DCM:MeOH=5:1) and further purified by prep-HPLC to give Compound 178 (5 mg, yield 4.7%) as a white solid. ¹H NMR (400 MHz, CD₃OD) δ 8.53 (s, 1H), 8.27-8.20 (m, 2H), 7.81 (dd, J=12.1, 5.9 Hz, 2H), 7.59-7.49 (m, 3H), 7.38-7.34 (m, 2H), 5.36-5.25 (m, 1H), 4.76-4.57 (m, 1H), 4.34-4.06 (m, 5H), 3.07 (d, J=18.2 Hz, 3H), 2.99-2.70 (m, 1H), 2.48 (ddd, J=20.3, 14.2, 7.2 Hz, 1H), 1.64 (dd, J=30.5, 7.0 Hz, 3H), 1.49 (s, 6H). LC/MS (ESI) m/z:622 (M+H)⁺.

### Scheme 170: Synthesis of (S)—N—((R)-1-(4-carbamimidoylthiophen-2-yl)ethyl)-7-((9,9-difluoro-1-methyl-9H-fluorene-3-carbonyl)glycyl)-1,4-dioxa-7-azaspiro[4.4]nonane-8-carboxamide (Compound 179)

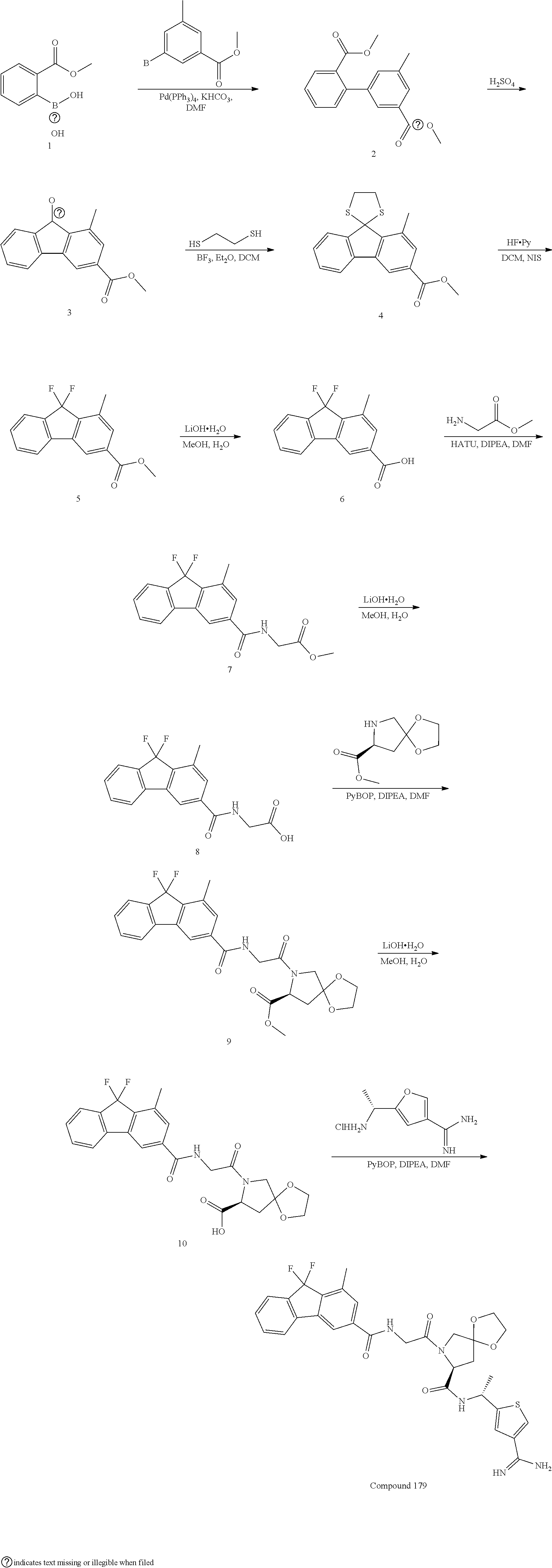

### Step 1: dimethyl 5′-methyl-[1,1′-biphenyl]-2,3′-dicarboxylate (2)

To a mixture of (2-(methoxycarbonyl)phenyl)boronic acid (918 mg, 5.11 mmol) and methyl 3-bromo-5-methylbenzoate (0.9 g, 3.93 mmol) in DMF (15 mL) was added KHCO₃ (1.76 g, 23.0 mmol) and Pd(PPh₃)₄ (135 mg, 0.12 mmol) at 25° C. under N₂ atmosphere, degassed under N₂ atmosphere for three times and stirred at 90° C. for 4 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give dimethyl 3-methyl-[1,1′-biphenyl]-2,3′-dicarboxylate (600 mg, yield 53.5%) as a yellow solid. LCMS (ESI) m/z: 285 (M+H)⁺.

### Step 2: methyl 1-methyl-9-oxo-9H-fluorene-3-carboxylate (3)

To a solution of dimethyl 5′-methyl-[1,1′-biphenyl]-2,3′-dicarboxylate (600 mg, 2.11 mmoL) in H₂SO₄ (12 mL) and stirred at 50° C. for 2 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give methyl 1-methyl-9-oxo-9H-fluorene-3-carboxylate (260 mg, yield 49.1%) as a yellow solid. LC/MS (ESI) m/z: 253 (M+H)⁺.

### Step 3: methyl 1-methylspiro[fluorene-9,2′-[1,3]dithiolane]-3-carboxylate (4)

To a solution of methyl 1-methyl-9-oxo-9H-fluorene-3-carboxylate (100 mg, 0.40 mmol) in DCM (2.0 mL) was added ethane-1,2-dithiol (188 mg, 1.98 mmol) and BF₃·Et₂O (0.2 mL, 98%) drop-wisely at 0° C. under N₂ atmosphere and stirred for 3 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give methyl 1-methylspiro[fluorene-9,2′-[1,3]dithiolane]-3-carboxylate (120 mg, yield 92.3%) as a colorless oil.

### Step 4: methyl 9,9-difluoro-1-methyl-9H-fluorene-3-carboxylate (5)

To a solution of NIS (576 mg, 2.52 mmol) in DCM (1.0 mL) was added HF/Pyridine (0.2 mL, 65%) drop-wisely at 0° C. under N₂ atmosphere and stirred for 15 minutes. The mixture was added drop-wisely a solution of methyl 1-methylspiro[fluorene-9,2′-[1,3]dithiolane]-3-carboxylate (120 mg, 0.36 mmol) in DCM (1.0 mL) and stirred overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-5% EtOAc in PE) to give methyl 9,9-difluoro-1-methyl-9H-fluorene-3-carboxylate (43 mg, yield 43.0%) as a white solid. LC/MS (ESI) m/z: 275 (M+H)⁺.

### Step 5: 9,9-difluoro-1-methyl-9H-fluorene-3-carboxylic acid (6)

To a solution of methyl 9,9-difluoro-1-methyl-9H-fluorene-3-carboxylate (43 mg, 0.16 mmol) in MeOH/H₂O (2 mL, 1/1) was added LiOH·H₂O (20 mg, 0.48 mmol) and stirred at room temperature for 2 hours. The mixture was acidified with 1 N aq·HCl to pH-3 and extracted with EtOAc twice. The combined organic layers were washed with brine, dried over anhydrous Na₂SO₄, filtered, and concentrated to dryness under reduced pressure to give 9,9-difluoro-1-methyl-9H-fluorene-3-carboxylic acid (40 mg, yield 97.6%) as a white solid. LC/MS (ESI) m/z: 261 (M+H)⁺.

### Step 6: methyl (9,9-difluoro-1-methyl-9H-fluorene-3-carbonyl)glycinate (7)

To a mixture of 9,9-difluoro-1-methyl-9H-fluorene-3-carboxylic acid (51 mg, 0.20 mmol) and methyl glycinate (37 mg, 0.30 mmol) in DMF (3 mL) was added HATU (89 mg, 0.24 mmol) and DIPEA (156 mg, 1.20 mmol) under N₂ atmosphere at room temperature and the reaction mixture was stirred for 2 hours. The mixture was diluted with EtOAc and washed with water and brine. The organic layers were dried over anhydrous Na₂SO₄, filtered, and concentrated to dryness under reduced pressure. The residue was purified by column chromatography (silica gel, 0-30% EtOAc in PE) to give methyl (9,9-difluoro-1-methyl-9H-fluorene-3-carbonyl)glycinate (60 mg, yield 92.3%) as a white solid. LCMS (ESI) m/z: 332 (M+H)⁺.

### Step 7: (9,9-difluoro-1-methyl-9H-fluorene-3-carbonyl)glycine (6)

To a solution of methyl (9,9-difluoro-1-methyl-9H-fluorene-3-carbonyl)glycinate (70 mg, 0.21 mmol) in MeOH/H₂O (4 mL, 3/1) was added LiOH·H₂O (27 mg, 0.63 mmol) and stirred at room temperature for 2 hours. The mixture was acidified with 1 N aq·HCl to pH-3 and extracted with EtOAc twice. The combined organic layers were washed with brine, dried over anhydrous Na₂SO₄, filtered, and concentrated to dryness under reduced pressure to give (9,9-difluoro-1-methyl-9H-fluorene-3-carbonyl)glycine (53 mg, yield 79.1%) as a white solid. LC/MS (ESI) m/z: 318 (M+H)⁺.

### Step 8: methyl (S)-7-((9,9-difluoro-1-methyl-9H-fluorene-3-carbonyl)glycyl)-1,4-dioxa-7-azaspiro[4.4]nonane-6-carboxylate (9)

To a mixture of (9,9-difluoro-1-methyl-9H-fluorene-3-carbonyl)glycine (53 mg, 0.16 mmol) and methyl (S)-1,4-dioxa-7-azaspiro[4.4]nonane-8-carboxylate (31 mg, 0.16 mmol) in DMF (3 mL) was added T₃P (319 mg, 0.48 mmol, 50% in EtOAc) and DIPEA (133 mg, 0.96 mmol) under N₂ atmosphere and the reaction mixture was stirred for 2 hours. The mixture was diluted with EtOAc and washed with water and brine. The organic layers were dried over anhydrous Na₂SO₄, filtered, and concentrated to dryness under reduced pressure. The residue was purified by column chromatography (silica gel, 0-5% MeOH in DCM) to give methyl (S)-7-((9,9-difluoro-1-methyl-9H-fluorene-3-carbonyl)glycyl)-1,4-dioxa-7-azaspiro[4.4]nonane-8-carboxylate (35 mg, yield 80.2%) as a colorless oil. LCMS (ESI) m/z: 487 (M+H)⁺.

### Step 9: (S)-7-((9,9-difluoro-1-methyl-9H-fluorene-3-carbonyl)glycyl)-1,4-dioxa-7-azaspiro[4.4]nonane-6-carboxylic acid (10)

To a solution of methyl (S)-7-((9,9-difluoro-1-methyl-9H-fluorene-3-carbonyl)glycyl)-1,4-dioxa-7-azaspiro[4.4]nonane-8-carboxylate (65 mg, 0.13 mmol) in MeOH/H₂O (1.0 mL, 1/1) was added LiOH·H₂O (8 mg, 0.20 mmol) and stirred at room temperature for 2 hours. The mixture was acidified with 1 N aq·HCl to pH-3 and extracted with EtOAc twice. The combined organic layers were washed with brine, dried over anhydrous Na₂SO₄, filtered, and concentrated to dryness under reduced pressure to give (S)-7-((9,9-difluoro-1-methyl-9H-fluorene-3-carbonyl)glycyl)-1,4-dioxa-7-azaspiro[4.4]nonane-8-carboxylic acid (60 mg, yield 95.2%) as a colorless oil. LC/MS (ESI) m/z: 473 (M+H)⁺.

### Step 10: (S)—N—((R)-1-(4-carbamimidoylthiophen-2-yl)ethyl)-7-((9,9-difluoro-1-methyl-9H-fluorene-3-carbonyl)glycyl)-1,4-dioxa-7-azaspiro[4.4]nonane-8-carboxamide (Compound 179)

To a mixture of (S)-7-((9,9-difluoro-1-methyl-9H-fluorene-3-carbonyl)glycyl)-1,4-dioxa-7-azaspiro[4.4]nonane-8-carboxylic acid (60 mg, 0.13 mmol) and (R)-5-(1-aminoethyl)thiophene-3-carboximidamide hydrochloride (46 mg, 0.19 mmol) in DMF (3 mL) was added PyBOP (66.1 mg, 0.13 mmol) and DIPEA (117 mg, 0.78 mmol) under N₂ atmosphere and the reaction mixture was stirred for 2 hours. The mixture was diluted with EtOAc and washed with water and brine. The organic layers were dried over anhydrous Na₂SO₄, filtered, and concentrated to dryness under reduced pressure. The residue was purified by pre-TLC (silica gel, 0-10% MeOH in DCM) and further purified by prep-HPLC to give Compound 179 (13 mg, yield 16.5%) as a white solid. ¹H NMR (400 MHz, CD₃OD) δ 8.55 (s, 1H), 8.23 (d, J=18.6 Hz, 1H), 7.97 (s, 1H), 7.71 (d, J=6.7 Hz, 1H), 7.64 (d, J=9.7 Hz, 2H), 7.55 (dd, J=17.0, 9.3 Hz, 2H), 7.43 (t, J=7.6 Hz, 1H), 5.29 (q, J=6.7 Hz, 1H), 4.63-4.54 (m, 1H), 4.27-4.12 (m, 2H), 4.04-3.96 (m, 4H), 3.80 (q, J=10.8 Hz, 2H), 2.56 (d, J=4.1 Hz, 3H), 2.44 (dd, J=13.1, 8.9 Hz, 1H), 2.21 (dd, J=13.1, 6.6 Hz, 1H), 1.61 (t, J=19.1 Hz, 3H). LCMS (ESI) m/z: 624 (M+H)⁺.

### Scheme 171: Synthesis of (S)—N—((R)-1-(4-carbamimidoylthiophen-2-yl)ethyl)-7-((9,9-difluoro-6-methyl-9H-fluorene-3-carbonyl)glycyl)-1,4-dioxa-7-azaspiro[4.4]nonane-8-carboxamide (Compound 180)

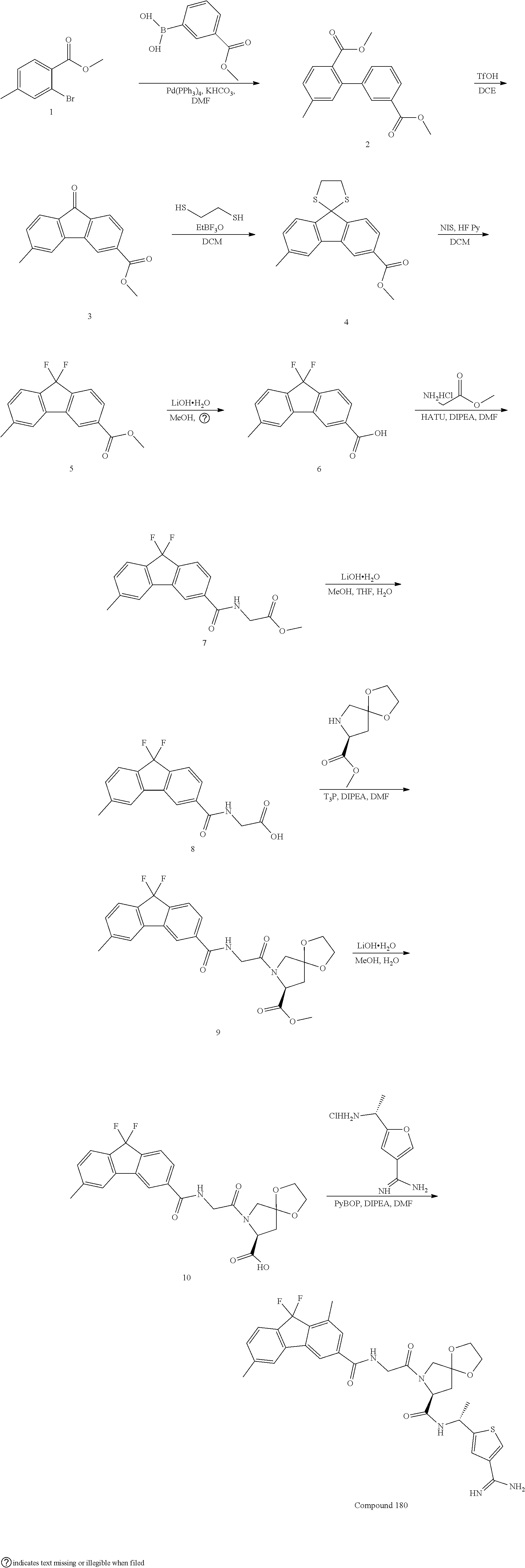

Compound 180 was prepared as a white solid from methyl 2-bromo-4-methylbenzoate, (3-(methoxycarbonyl)phenyl)boronic acid, methyl (S)-1,4-dioxa-7-azaspiro[4.4]nonane-8-carboxylate, and (R)-5-(1-aminoethyl)thiophene-3-carboximidamide hydrochloride based on the procedures set forth in Scheme 170. ¹H NMR (400 MHz, CD₃OD) δ 8.55 (s, 1H), 8.23 (t, J=8.0 Hz, 1H), 8.15 (s, 1H), 7.86 (d, J=7.8 Hz, 1H), 7.69 (d, J=7.9 Hz, 1H), 7.60 (d, J=9.5 Hz, 1H), 7.52 (d, J=6.1 Hz, 2H), 7.27 (d, J=7.7 Hz, 1H), 5.29 (q, J=6.8 Hz, 1H), 4.60-4.55 (m, 1H), 4.20 (t, J=12.3 Hz, 2H), 4.03-3.95 (m, 4H), 3.81 (q, J=11.0 Hz, 2H), 2.47-2.41 (m, 4H), 2.21 (dd, J=12.9, 6.8 Hz, 1H), 1.61 (t, J=18.8 Hz, 3H). LCMS (ESI) m/z: 624 (M+H)⁺.

### Scheme 172: Synthesis of (S)—N—((R)-1-(4-carbamimidoylthiophen-2-yl)ethyl)-7-((4-fluoro-5-(4-fluorophenyl)picolinoyl)glycyl)-1,4-dioxa-7-azaspiro[4.4]nonane-8-carboxamide (Compound 181)

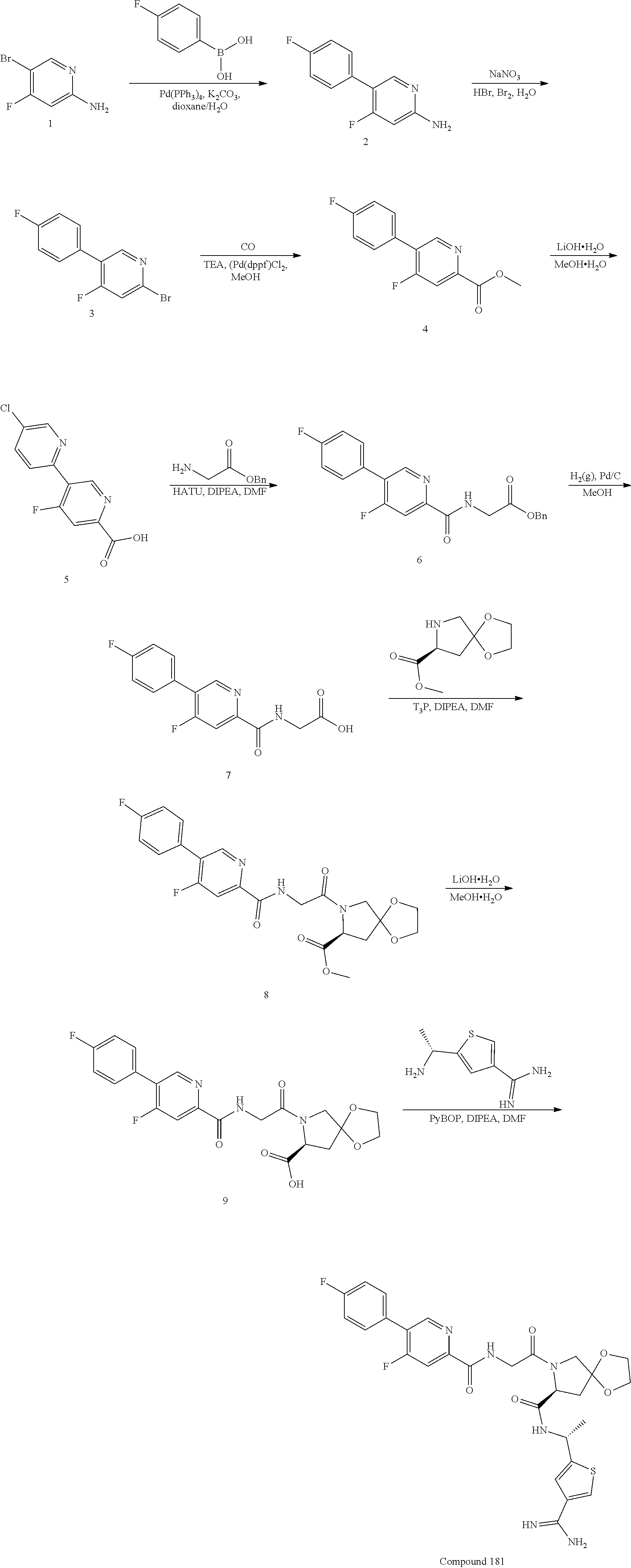

### Step 1: 4-fluoro-5-(4-fluorophenyl)pyridin-2-amine (2)

To a mixture of 5-bromo-4-fluoropyridin-2-amine (316 mg, 1.65 mmol) and (4-fluorophenyl)boronic acid (301 mg, 2.15 mmol) in 1,4-dioxane (4 mL) and water (1 mL) was added K₂CO₃ (685 mg, 4.95 mmol), Pd(PPh₃)₄ (96 mg, 0.08 mmol). The mixture was degassed under N₂ atmosphere and stirred at 100° C. overnight. The mixture was diluted with water and extracted with EtOAc twice. The combined organic layers were washed with brine, dried over anhydrous Na₂SO₄, filtered, and concentrated under reduced pressure to dryness. The residue was purified by column chromatography (silica gel, 0-20% EtOAc in PE) to give 4-fluoro-5-(4-fluorophenyl)pyridin-2-amine (285 mg, yield 83.8%) as a white solid. LCMS (ESI) m/z: 207 (M+H)⁺.

### Step 2: 2-bromo-4-fluoro-5-(4-fluorophenyl)pyridine (3)

To a solution of 4-fluoro-5-(4-fluorophenyl)pyridin-2-amine (185 mg, 0.89 mmol) in HBr (2 mL, 48% in H₂O) was added Br₂ (466 mg, 2.69 mmol) and a solution of NaNO₂ (224 mg, 2.23 mmol) in H₂O dropwise at 0° C. and the mixture was stirred at 0° C. for 0.5 hours under N₂ atmosphere. The mixture was quenched with aq. NaOH and extracted with EtOAc, washed with brine, dried over anhydrous Na₂SO₄, filtered, and concentrated under reduced pressure to dryness. The residue was purified by flash chromatography (silica gel, 0-5% EtOAc in PE) to give 2-bromo-4-fluoro-5-(4-fluorophenyl)pyridine (120 mg, yield 49.8%) as a white solid. LC/MS (ESI) (m/z): 270/272 (M+H)⁺.

### Step 3: methyl 4-fluoro-5-(4-fluorophenyl)picolinate (4)

To a mixture of 2-bromo-4-fluoro-5-(4-fluorophenyl)pyridine (150 mg, 0.56 mmol) and TEA (0.23 mL, 1.68 mmol) in MeOH (6 mL) was added Pd(dppf)Cl₂ (41 mg, 0.06 mmol) and the mixture was stirred under CO atmosphere at 75° C. for 16 hours. The mixture was diluted with water and extracted with DCM twice. The combined organic layers were dried over anhydrous Na₂SO₄, filtered, and concentrated to dryness under reduced pressure. The residue was purified by flash chromatography (silica gel, 0-20% EtOAc in PE) to give methyl 4-fluoro-5-(4-fluorophenyl)picolinate (70 mg, yield 50.7%) as a white solid. LC/MS (ESI) m/z: 250 (M+H)⁺.

### Step 4: 4-fluoro-5-(4-fluorophenyl)picolinic acid (5)

To a solution of methyl 4-fluoro-5-(4-fluorophenyl)picolinate (113 mg, 0.45 mmol) in MeOH (3 mL), water (1 mL) was added LiOH·H₂O (56 mg, 1.35 mmol) at 0° C. and the mixture was stirred at room temperature for 2 hours. The mixture was acidified with 1N aq·HCl to pH˜3, extracted with EtOAc twice. The combined organic layers were washed with brine, dried over anhydrous Na₂SO₄, filtered, and concentrated under reduced pressure to give 4-fluoro-5-(4-fluorophenyl)picolinic acid (105 mg, yield 98.1%) as a white solid, which was used directly in the next reaction. LC/MS (ESI) (m/z): 236 (M+H)⁺.

### Step 5: benzyl (4-fluoro-5-(4-fluorophenyl)picolinoyl)glycinate (6)

To a mixture of 4-fluoro-5-(4-fluorophenyl)picolinic acid (110 mg, 0.47 mmol) and benzyl glycinate (142 g, 0.70 mmol) in DMF (3 mL) was added HATU (213 mg, 0.56 mmol) and DIPEA (0.46 mL, 2.82 mmol) under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-20% EtOAc in PE) to give benzyl (4-fluoro-5-(4-fluorophenyl)picolinoyl)glycinate (142 mg, yield 79.3%) as a white solid. LC/MS (ESI) m/z: 383 (M+H)⁺.

### Step 6: (4-fluoro-5-(4-fluorophenyl)picolinoyl)glycine (7)

To a solution of benzyl (4-fluoro-5-(4-fluorophenyl)picolinoyl)glycinate (142 mg, 0.37 mmol) in MeOH (5 mL) was added Pd/C (10 mg, 10% wt.), the mixture was degassed under N₂ atmosphere for three times and stirred under a H₂ balloon at 25° C. for 2 hours. The mixture was filtered, and the filtrate was concentrated to dryness to give (4-fluoro-5-(4-fluorophenyl)picolinoyl)glycine (108 mg, yield 99.8%) as a white solid. LC/MS (ESI) m/z: 293 (M+H)⁺.

### Step 7: methyl (S)-7-((4-fluoro-5-(4-fluorophenyl)picolinoyl)glycyl)-1,4-dioxa-7-azaspiro[4.4]nonane-8-carboxylate (8)

To a mixture of (4-fluoro-5-(4-fluorophenyl)picolinoyl)glycine (108 mg, 0.37 mmol) and methyl (S)-1,4-dioxa-7-azaspiro[4.4]nonane-8-carboxylate (69 mg, 0.37 mmol) in DMF (3 mL) was added DIPEA (0.37 mL, 2.22 mmol) and T₃P (706 mg, 1.11 mmol, 50% wt. in EtOAc) under N₂ atmosphere and the mixture was stirred at room temperature for 1 hours. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-5% MeOH in DCM) to give methyl (S)-7-((4-fluoro-5-(4-fluorophenyl)picolinoyl)glycyl)-1,4-dioxa-7-azaspiro[4.4]nonane-8-carboxylate (156 mg, yield 91.8%) as a colorless oil. LC/MS (ESI) m/z: 462 (M+H)⁺.

### Step 8: (S)-7-((4-fluoro-5-(4-fluorophenyl)picolinoyl)glycyl)-1,4-dioxa-7-azaspiro[4.4]nonane-8-carboxylic acid (9)

To a solution of methyl (S)-7-((4-fluoro-5-(4-fluorophenyl)picolinoyl)glycyl)-1,4-dioxa-7-azaspiro[4.4]nonane-8-carboxylate (156 mg, 0.34 mmol) in MeOH (3 mL), water (1 mL) was added LiOH·H₂O (21 mg, 0.51 mmol) at 0° C. and the mixture was stirred at room temperature for 2 hours. The mixture was acidified with 1 N aq·HCl to pH˜3, extracted with EtOAc twice. The combined organic layers were washed with brine, dried over anhydrous Na₂SO₄, filtered, and concentrated under reduced pressure to give (S)-7-((4-fluoro-5-(4-fluorophenyl)picolinoyl)glycyl)-1,4-dioxa-7-azaspiro[4.4]nonane-8-carboxylic acid (130 mg, yield 86.1%) as a white solid, which was used directly in the next reaction. LC/MS (ESI) (m/z): 448 (M+H)⁺.

### Step 9: (S)—N—((R)-1-(4-carbamimidoylthiophen-2-yl)ethyl)-7-((4-fluoro-5-(4-fluorophenyl)picolinoyl) glycyl)-1,4-dioxa-7-azaspiro[4.4]nonane-8-carboxamide (Compound 181)

To a solution of (S)-7-((4-fluoro-5-(4-fluorophenyl)picolinoyl)glycyl)-1,4-dioxa-7-azaspiro[4.4]nonane-8-carboxylic acid (80 mg, 0.18 mmol) and (R)-5-(1-aminoethyl)thiophene-3-carboximidamide (55 mg, 0.27 mmol) in DMF (3 mL) was added DIPEA (141 mg, 1.08 mmol) and PyBOP (11 mg, 0.22 mmol) at 0° C. under N₂ atmosphere and the mixture was stirred at 25° C. for 1 hour. The reaction mixture was diluted with water and extracted with EtOAc twice. The combined organic layers were washed with brine, dried over anhydrous Na₂SO₄, filtered, and concentrated to dryness under reduced pressure. The residue was purified by pre-TLC and further purified by prep-HPLC to give Compound 181 (5 mg, yield 4.7%) as a white solid. ¹H NMR (400 MHz, CD₃OD) δ 8.78 (d, J=9.8 Hz, 1H), 8.23 (s, 1H), 7.91 (d, J=10.9 Hz, 1H), 7.68 (s, 2H), 7.56 (s, 1H), 7.29 (t, J=8.7 Hz, 2H), 5.28 (d, J=6.7 Hz, 1H), 4.57 (t, J=7.7 Hz, 1H), 4.26 (q, J=17.2 Hz, 2H), 4.03-3.96 (m, 4H), 3.77 (dd, J=24.0, 10.5 Hz, 2H), 2.49-2.40 (m, 1H), 2.21 (dd, J=12.9, 6.9 Hz, 1H), 1.59 (d, J=7.0 Hz, 3H). LCMS (ESI) (m/z): 599 (M+H)⁺.

### Scheme 173: Synthesis of (S)—N—((R)-1-(4-carbamimidoylthiophen-2-yl)ethyl)-7-((9,9-difluoro-8-methyl-9H-fluorene-3-carbonyl)glycyl)-1,4-dioxa-7-azaspiro[4.4]nonane-8-carboxamide (Compound 182)

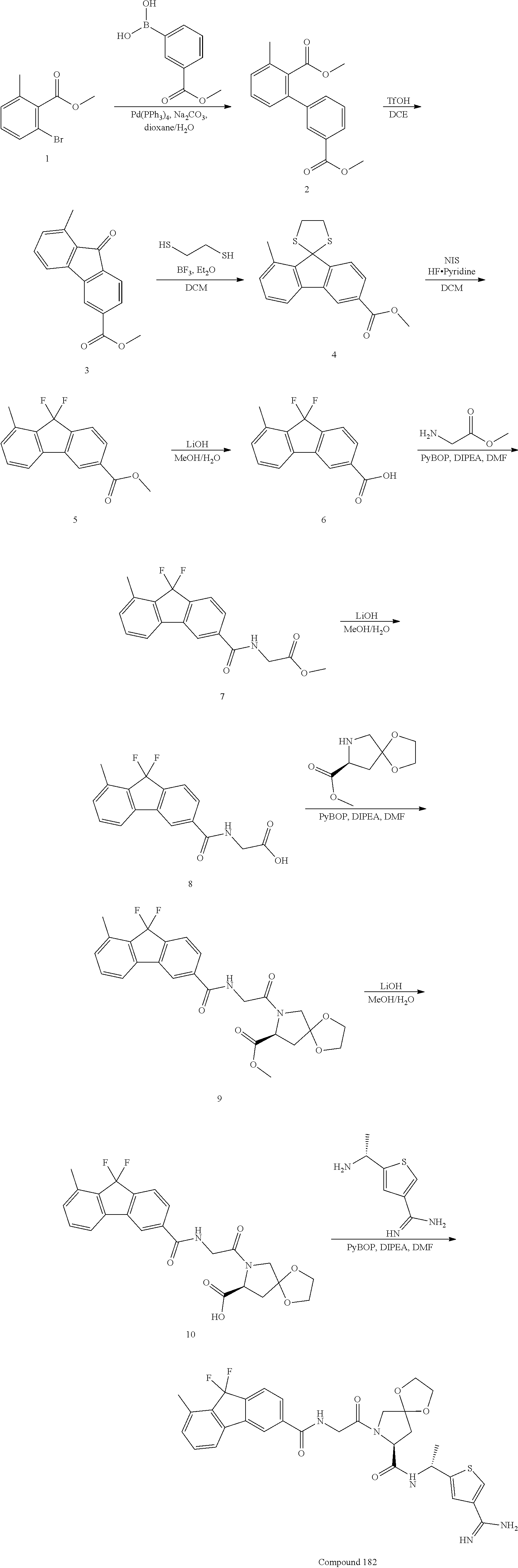

Compound 182 was prepared as a white solid form methyl 2-bromo-6-methylbenzoate, (3-(methoxycarbonyl)phenyl)boronic acid, methyl glycinate, methyl (S)-1,4-dioxa-7-azaspiro[4.4]nonane-8-carboxylate, and (R)-5-(1-aminoethyl)thiophene-3-carboximidamide based on the procedures set forth in Scheme 170. ¹H NMR (400 MHz, CD₃OD) δ 8.23 (s, 1H), 8.15 (s, 1H), 7.86 (d, J=7.6 Hz, 1H), 7.70 (d, J=8.0 Hz, 1H), 7.57-7.51 (m, 2H), 7.44 (t, J=7.7 Hz, 1H), 7.21 (d, J=7.7 Hz, 1H), 5.28 (d, J=7.3 Hz, 1H), 4.57 (t, J=7.4 Hz, 1H), 4.21 (q, J=16.9 Hz, 2H), 4.02-3.95 (m, 4H), 3.80 (q, J=10.8 Hz, 2H), 2.52 (s, 3H), 2.44 (dd, J=13.3, 8.7 Hz, 1H), 2.21 (dd, J=13.1, 6.6 Hz, 1H), 1.58 (d, J=6.9 Hz, 3H). LCMS (ESI) m/z: 624 (M+H)⁺.

### Scheme 174: (S)—N-((4-carbamimidoylthiophen-2-yl)methyl)-7-((4′-(methylsulfonyl)-[1,1′-biphenyl]-4-carbonyl)glycyl)-1,4-dioxa-7-azaspiro[4.4]nonane-8-carboxamide (Compound 183)

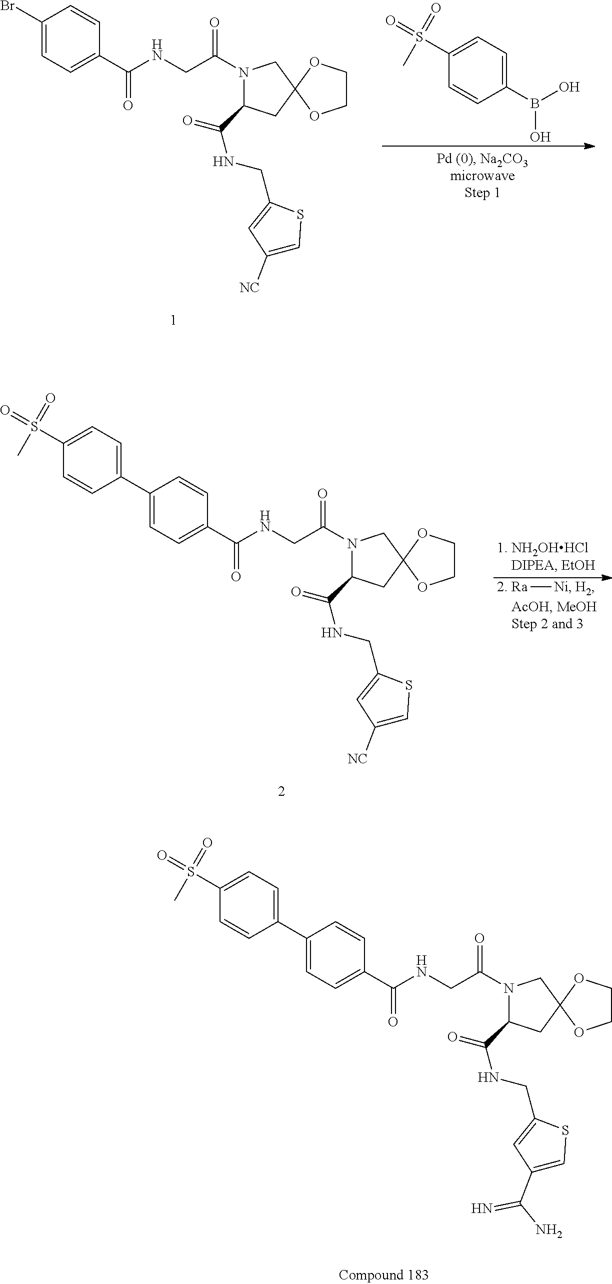

Compound 183 was prepared from (4-methylsulfonylphenyl)boronic acid and (8S)-7-[2-[(4-bromobenzoyl)amino]acetyl]-N-[(4-cyano-2-thienyl)methyl]-1,4-dioxa-7-azaspiro[4.4]nonane-8-carboxamide based on the procedures set forth in Scheme 103. LC/MS (ESI) m/z: 626 (M+H)⁺. ¹H NMR (400 MHz, DMSO) δ 8.80 (s, 1H), 8.65 (s, 1H), 8.44 (s, 1H), 8.01 (d, J=10.3 Hz, 2H), 7.97-7.83 (m, 1H), 7.77 (s, 1H), 7.11 (s, 1H), 4.51-4.45 (m, 3H), 4.41 (t, J=7.7 Hz, 1H), 4.20 (d, J=15.9 Hz, 1H), 3.96-3.92 (m, 4H), 3.81 (d, J=10.7 Hz, 1H), 3.75-3.60 (m, 1H), 3.27 (s, 3H), 2.33 (t, J=11.1 Hz, 1H), 2.07 (dd, J=12.9, 6.9 Hz, 1H).

### Scheme 175: (S)—N-((4-(4-aminothiazol-2-yl)thiophen-2-yl)methyl)-7-((4-(4-fluorophenoxy)benzoyl)glycyl)-1,4-dioxa-7-azaspiro[4.4]nonane-8-carboxamide (Compound 184)

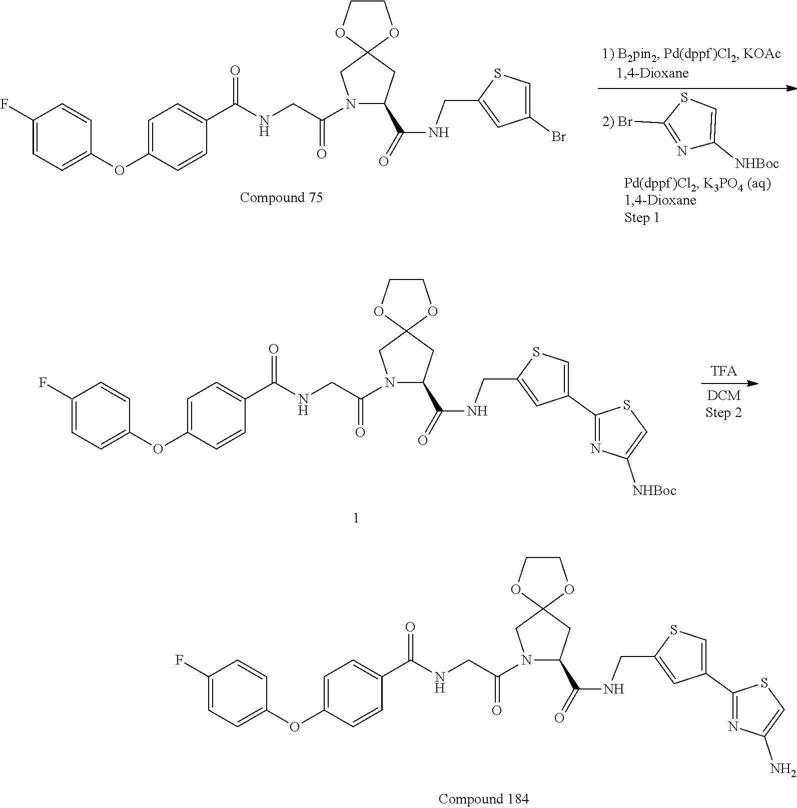

### Step 1: tert-butyl (S)-(2-(5-((7-((4-(4-fluorophenoxy)benzoyl)glycyl)-1,4-dioxa-7-azaspiro[4.4]nonane-6-carboxamido)methyl)thiophen-3-yl)thiazol-4-yl)carbamate (1)

To a solution of Compound 75 (0.040 g, 0.065 mmol, 1.0 equiv.) in 1,4-Dioxane (3.00 mL) was added Bis(pinacolato)diboron (0.035 g, 0.14 mmol, 2.1 equiv.) and Potassium acetate (0.020 g, 0.20 mmol, 3.2 equiv.). Ar gas was passed through the solution for 2 min followed by the addition of Pd(dppf)Cl₂·CH₂Cl₂ (0.010 g, 0.012 mmol, 0.19 equiv.). The mixture was de-gassed once again by purging with Ar. The tube was sealed and placed in a microwave synthesizer for 30 mins at 110° C. LC/MS indicated the presence of the borylated product. To this mixture was added tert-butyl (2-bromothiazol-4-yl)carbamate (0.045 g, 0.16 mmol, 2.5 equiv.) followed by Potassium phosphate tribasic solution in H₂O (0.10 mL, 0.20 mmol, 2.00 mol/L, 3.1 equiv.) and Pd(dppf)Cl₂·CH₂Cl₂ (0.010 g, 0.012 mmol, 0.19 equiv.). It was then further degassed by passing Ar through for 2 mins. The tube was sealed again, and it was irradiated with MW for another 30 mins. LC/MS showed the presence of the desired product. Aqueous phase from the reaction mixture was removed and it was then passed through a pad of celite. The filtrate was concentrated and the residue was purified by column chromatography using 0-10% MeOH in DCM as eluent to obtain the desired product tert-butyl (S)-(2-(5-((7-((4-(4-fluorophenoxy)benzoyl)glycyl)-1,4-dioxa-7-azaspiro[4.4]nonane-8-carboxamido)methyl)thiophen-3-yl)thiazol-4-yl)carbamate, 1 (25.0 mg, 0.0339 mmol, 52% yield) as a white solid.LC/MS (ESI) m/z: 738 (M+H)⁺.

### Step 2: (S)—N-((4-(4-aminothiazol-2-yl)thiophen-2-yl)methyl)-7-((4-(4-fluorophenoxy)benzoyl)glycyl)-1,4-dioxa-7-azaspiro[4.4]nonane-8-carboxamide (Compound 184)

To the solution of 1 (0.025 g, 0.0339 mmol, 1.0 equiv.) in Dichloromethane (5.00 mL) was added Trifluoroacetic acid (2.00 mL, 26.50 mmol, 650.0 equiv.). The mixture was stirred for 2 h. It was then concentrated, and the residue was purified by ISCO column chromatographic purification system (12 g gold) using 0-10% MeOH in DCM as eluent to obtain desired product Compound 184 (0.003 g, 0.005 mmol, 10% yield) as a white solid. LC/MS (ESI) m/z: 638 (M+H)⁺. ¹H NMR (400 MHz, DMSO) δ 2.04 (dd, J=13.0, 6.6 Hz, 1H), 2.29 (d, J=8.6 Hz, OH), 3.59-3.71 (m, 1H), 3.78 (d, J=10.8 Hz, 1H), 3.86-4.01 (m, 6H), 4.15 (dd, J=16.8, 5.6 Hz, 1H), 4.41 (dt, J=16.0, 6.7 Hz, 2H), 4.51 (d, J=6.7 Hz, 1H), 4.71 (s, OH), 5.35 (s, 1H), 6.68 (s, 2H), 7.01 (dd, J=8.3, 4.7 Hz, 2H), 7.15 (dd, J=9.1, 4.6 Hz, 2H), 7.28 (dd, J=16.8, 8.3 Hz, 3H), 7.75 (d, J=12.7 Hz, 1H), 7.88 (d, J=8.5 Hz, 2H), 8.46 (s, 0H), 8.48-8.60 (m, 2H).

### Scheme 176: (S)—N-((4-(5-aminothiazol-2-yl)thiophen-2-yl)methyl)-7-((4-(4-fluorophenoxy)benzoyl)glycyl)-1,4-dioxa-7-azaspiro[4.4]nonane-8-carboxamide (Compound 185)

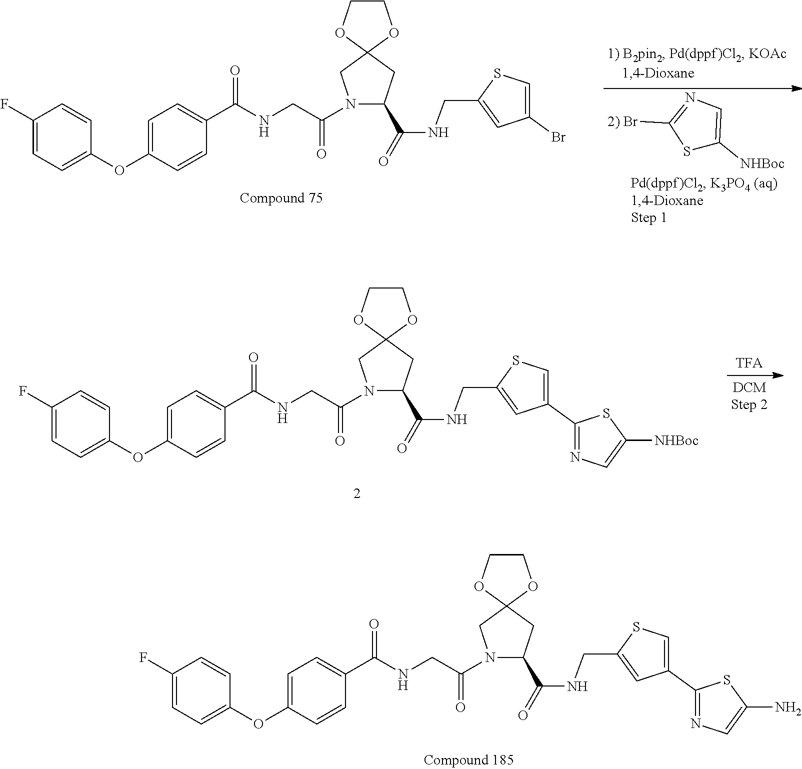

Compound 185 was prepared as a white solid from Compound 75 and and tert-butyl (2-bromothiazol-5-yl)carbamate based on the procedures set forth in Scheme 175. LC/MS (ESI) m/z: 638 (M+H)⁺. ¹H NMR (400 MHz, DMSO) δ 1.98-2.11 (m, 1H), 2.26-2.35 (m, OH), 3.58-3.71 (m, 2H), 3.78 (d, J=10.8 Hz, 1H), 3.94 (dd, J=9.5, 5.7 Hz, 5H), 4.16 (dd, J=16.7, 5.7 Hz, 1H), 4.40 (s, 2H), 4.39-4.52 (m, 1H), 5.74 (s, 2H), 6.49 (s, 1H), 6.70 (s, 1H), 6.97-7.05 (m, 2H), 7.15 (dd, J=9.2, 4.5 Hz, 3H), 7.23-7.31 (m, 4H), 7.51 (d, J=10.7 Hz, 1H), 7.88 (d, J=8.4 Hz, 3H), 8.48 (t, J=6.0 Hz, 1H), 8.57 (d, J=6.2 Hz, 1H).

### Scheme 177: (S)—N-((4-(2-aminothiazol-4-yl)thiophen-2-yl)methyl)-7-((4-(4-fluorophenoxy)benzoyl)glycyl)-1,4-dioxa-7-azaspiro[4.4]nonane-8-carboxamide (Compound 186)

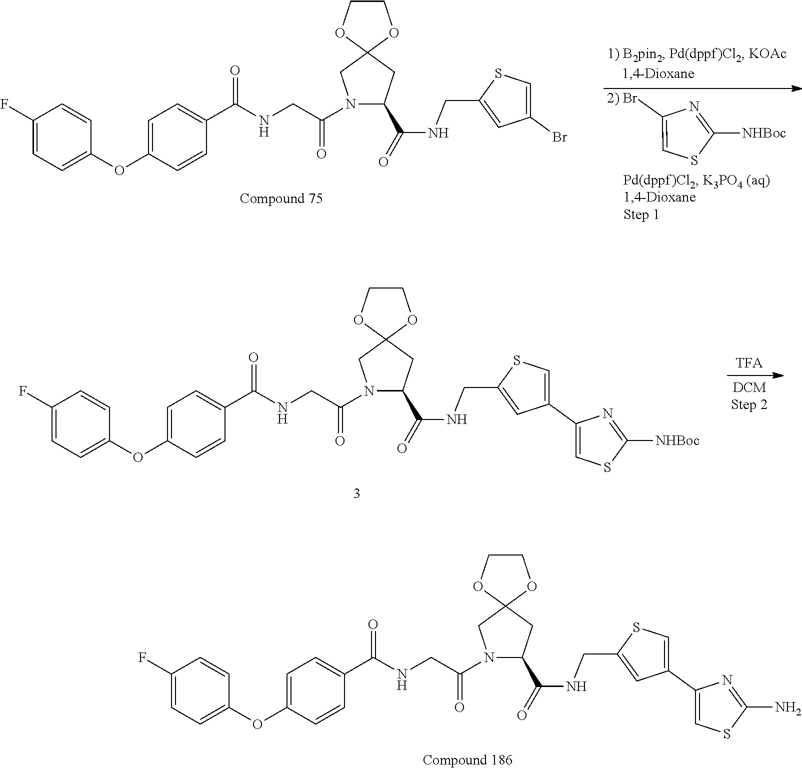

Compound 186 was prepared as a white solid from Compound 75 and tert-butyl (4-bromothiazol-2-yl)carbamate based on the procedures set forth in Scheme 175. LC/MS (ESI) m/z: 638 (M+H)⁺. ¹H NMR (400 MHz, DMSO) δ 2.04 (dd, J=13.1, 7.0 Hz, 1H), 2.26-2.34 (m, 1H), 3.58-3.73 (m, 1H), 3.79 (d, J=10.8 Hz, 1H), 3.94 (tt, J=15.0, 7.9 Hz, 5H), 4.06-4.21 (m, 1H), 4.31-4.52 (m, 3H), 6.59 (s, 1H), 6.68 (s, 1H), 6.93-7.05 (m, 4H), 7.15 (dd, J=9.1, 4.5 Hz, 2H), 7.28 (dd, J=16.5, 7.5 Hz, 3H), 7.42 (d, J=16.8 Hz, 1H), 7.88 (d, J=8.5 Hz, 2H), 8.50 (t, J=5.9 Hz, 1H), 8.59 (t, J=5.7 Hz, 1H).

### Scheme 178: (S)—N-((4-(2-aminothiazol-5-yl)thiophen-2-yl)methyl)-7-((4-(4-fluorophenoxy)benzoyl)glycyl)-1,4-dioxa-7-azaspiro[4.4]nonane-8-carboxamide (Compound 187)

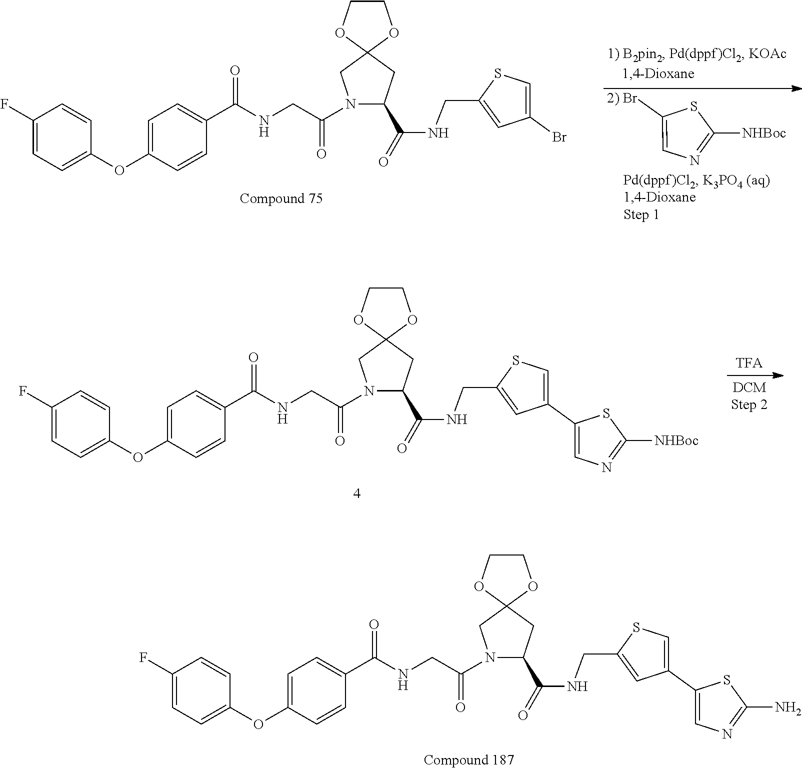

Compound 187 was prepared as a white solid from Compound 75 and tert-butyl (5-bromothiazol-2-yl)carbamate based on the procedures set forth in Scheme 175. LC/MS (ESI) m/z: 638 (M+H)⁺. ¹H NMR (400 MHz, DMSO) δ 2.04 (dd, J=13.1, 6.6 Hz, 1H), 2.31 (d, J=17.3 Hz, OH), 3.64 (t, J=11.6 Hz, 1H), 3.79 (d, J=10.9 Hz, 1H), 3.88-4.01 (m, 5H), 4.06-4.20 (m, 1H), 4.38 (d, J=6.4 Hz, 2H), 6.56 (s, 3H), 6.97-7.04 (m, 4H), 7.15 (p, J=5.1 Hz, 5H), 7.27 (t, J=8.7 Hz, 2H), 7.87 (d, J=8.6 Hz, 2H), 8.43-8.63 (m, 1H).

### Scheme 179: (S)—N-((4-carbamimidoylthiophen-2-yl)methyl)-7-((2′,5′-difluoro-[1,1′-biphenyl]-4-carbonyl)glycyl)-1,4-dioxa-7-azaspiro[4.4]nonane-8-carboxamide (Compound 188)

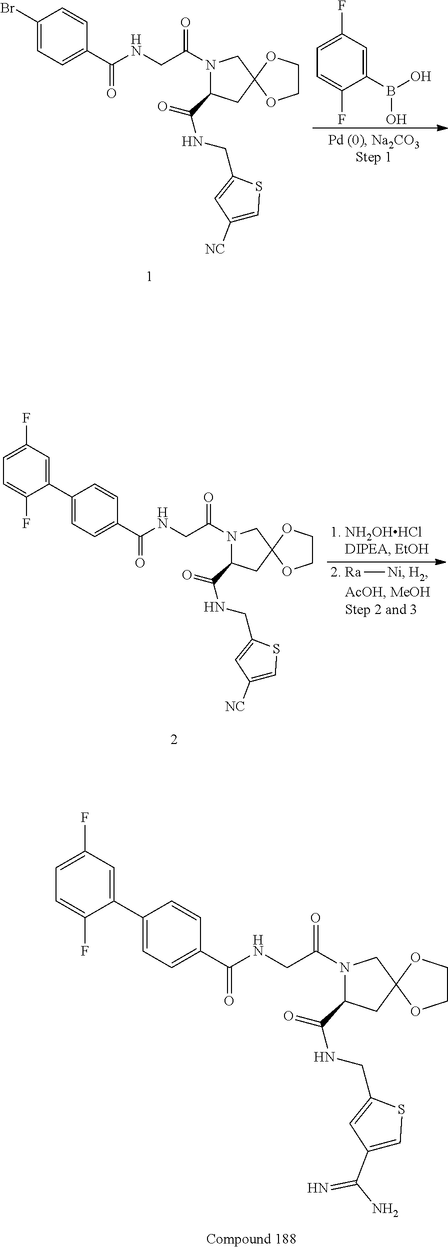

Compound 188 was prepared from (2,5-difluorophenyl)boronic acid and (8S)-7-[2-[(4-bromobenzoyl)amino]acetyl]-N-[(5-cyano-3-thienyl)methyl]-1,4-dioxa-7-azaspiro[4.4]nonane-8-carboxamide based on the procedures set forth in Scheme 103. LC/MS (ESI) m/z: 584 (M+H)⁺. ¹H NMR (400 MHz, DMSO-d₆) δ 9.01 (d, J=6.0 Hz, 1H), 8.77 (t, J=5.6 Hz, 2H), 8.63 (t, J=5.9 Hz, 2H), 8.34 (d, J=13.5 Hz, 2H), 7.97 (d, J=8.2 Hz, 3H), 7.69 (d, J=7.8 Hz, 3H), 7.57-7.45 (m, 3H), 7.31 (dq, J=8.9, 4.3 Hz, 2H), 4.72-470 (m, 4H), 4.52 (d, J=5.6 Hz, 2H), 4.16 (qd, J=16.6, 5.6 Hz, 2H), 3.74-3.56 (m, 3H), 2.32 (dd, J=13.2, 8.8 Hz, 1H), 2.05 (dd, J=13.0, 6.8 Hz, 1H).

### Scheme 180: (S)—N-((4-carbamimidoylthiophen-2-yl)methyl)-7-((3′,5′-difluoro-[1,1′-biphenyl]-4-carbonyl)glycyl)-1,4-dioxa-7-azaspiro[4.4]nonane-8-carboxamide (Compound 189)

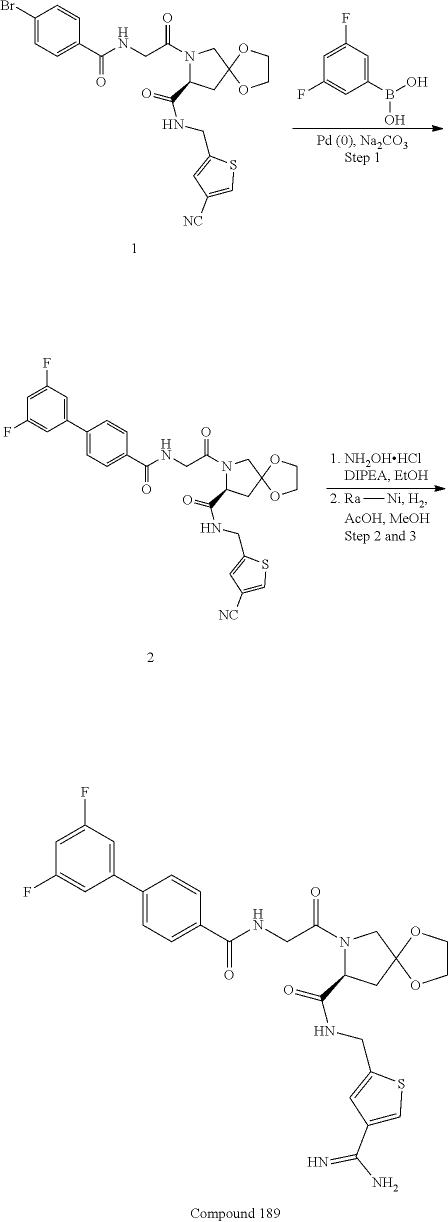

Compound 189 was prepared from (3,5-difluorophenyl)boronic acid and (8S)-7-[2-[(4-bromobenzoyl)amino]acetyl]-N-[(5-cyano-3-thienyl)methyl]-1,4-dioxa-7-azaspiro[4.4]nonane-8-carboxamide based on the procedures set forth in Scheme 103. LC/MS (ESI) m/z: 584 (M+H)⁺. ¹H NMR (400 MHz, DMSO-d₆) δ 8.97 (s, 1H), 8.77 (s, 2H), 8.60 (s, 1H), 8.46 (s, 2H), 8.31 (d, J=13.0 Hz, 1H), 7.97 (d, J=8.1 Hz, 2H), 7.88 (d, J=8.1 Hz, 2H), 7.61-7.49 (m, 2H), 7.46 (s, 1H), 7.28 (t, J=9.4 Hz, 2H), 4.71-4.51 (m, 1H), 4.47-4.34 (m, 2H), 4.19 (dd, J=16.7, 5.4 Hz, 2H), 4.02 (d, J=5.2 Hz, 1H), 4.00-3.83 (m, 2H), 3.80 (d, J=10.8 Hz, 2H), 3.68-3.56 (m, 1H), 2.32 (dd, J=13.5, 8.6 Hz, 1H), 2.11-1.95 (m, 1H).

### Scheme 181: Synthesis of (S)—N—((R)-1-(4-carbamimidoylthiophen-2-yl)ethyl)-7-((9,9-difluoro-7-methyl-9H-fluorene-3-carbonyl)glycyl)-1,4-dioxa-7-azaspiro[4.4]nonane-8-carboxamide (Compound 190)

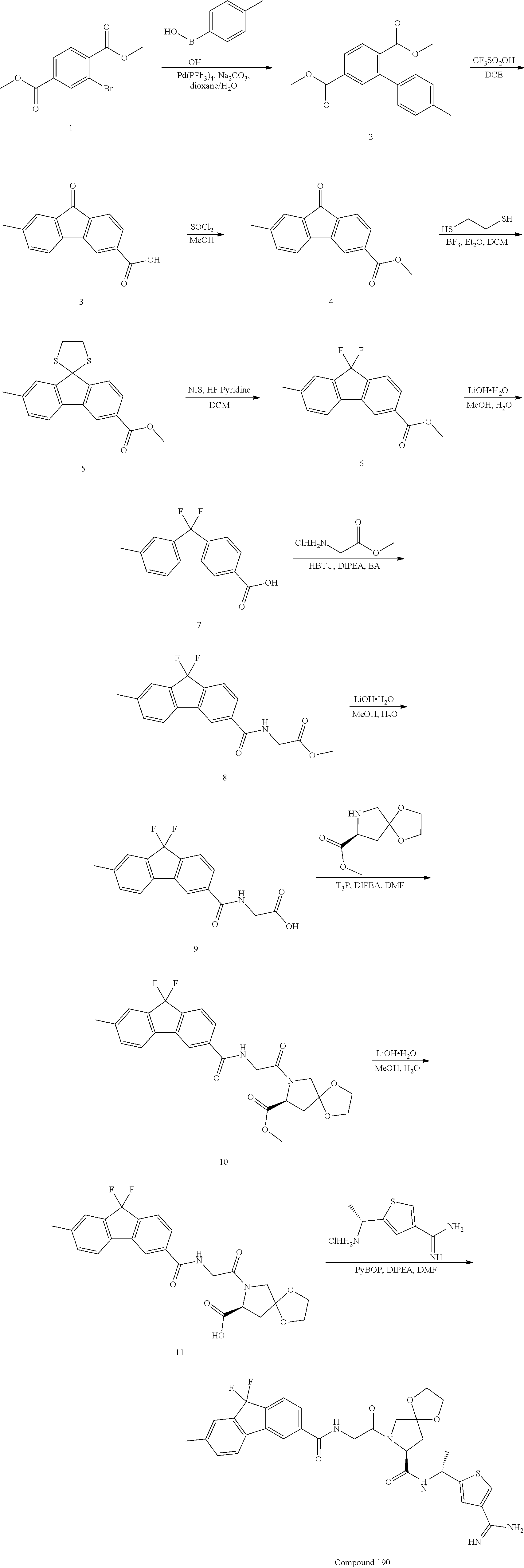

### Step 1: dimethyl 4′-methyl-[1,1′-biphenyl]-2,5-dicarboxylate (2)

To a mixture of dimethyl 2-bromoterephthalate (1.0 g, 3.66 mmol) in dioxane (8 mL) and water (2 mL) was added (4-methylphenyl)boronic acid (1.0 g, 7.32 mmol), Pd(PPh₃)₄ (208 mg, 0.18 mmol) and Na₂CO₃ (0.97 g, 9.15 mmol). The reaction mixture was degassed under N₂ atmosphere for three times and the reaction mixture was stirred at 85° C. for 2 hours. The mixture was diluted with water and extracted with EtOAc twice. The combined organic layers were washed with brine, dried over anhydrous Na₂SO₄, filtered, and concentrated under reduced pressure to dryness. The residue was purified by column chromatography on (silica gel, 0-20% EtOAc in PE) to give dimethyl 4′-methyl-[1,1′-biphenyl]-2,5-dicarboxylate (0.92 g, yield 88.4%) as a white solid. LCMS (ESI) m/z=285 (M+H)⁺.

### Step 2: 7-methyl-9-oxo-9H-fluorene-3-carboxylic acid (3)

To a solution of dimethyl 4′-methyl-[1,1′-biphenyl]-2,5-dicarboxylate (0.9 g, 3.17 mmol) in DCE (10 mL) was added trifluoromethanesulfonic acid (4.8 g, 32 mmol) and the reaction mixture was stirred at 85° C. under N₂ atmosphere overnight. The mixture was diluted with water and extracted with EtOAc twice. The combined organic layers were washed with brine, dried over anhydrous Na₂SO₄, filtered, and concentrated to dryness to give 7-methyl-9-oxo-9H-fluorene-3-carboxylic acid (640 mg, yield 84.7%) as a yellow solid. LCMS (ESI) m/z=239 (M+H)⁺.

### Step 3: methyl 7-methyl-9-oxo-9H-fluorene-3-carboxylate (4)

To a solution of 7-methyl-9-oxo-9H-fluorene-3-carboxylic acid (0.63 g, 2.64 mmol) in MeOH (10 mL) was added sulfurous dichloride (1.57 g, 13.2 mmol) and the reaction mixture was stirred at 85° C. under N₂ atmosphere overnight. The mixture was concentrated to dryness under reduced pressure. The residue was purified by column chromatography (silica gel, 0-30% EtOAc in PE) to give methyl 7-methyl-9-oxo-9H-fluorene-3-carboxylate (0.65 g, yield 97.6%) as a yellow solid. LCMS (ESI) m/z=253 (M+H)⁺.

### Step 4: methyl 2-methylspiro[fluorene-9,2′-[1,3]dithiolane]-6-carboxylate (5)

To a solution of methyl 7-methyl-9-oxo-9H-fluorene-3-carboxylate (0.63 g, 2.5 mmol) in DCM (10 mL) was added ethane-1,2-dithiol (1.18 g, 12.5 mmol) and BF₃·Et₂O (6 mL) at 0° C. The mixture was stirred at room temperature under N₂ atmosphere for 4 hours. The mixture was diluted with water and extracted with DCM. The combined organic layers were washed with brine, dried over anhydrous Na₂SO₄, filtered, and concentrated to dryness under reduced pressure. The residue was purified by flash chromatography (silica gel, 0-2% EtOAc in PE) to give methyl 2-methylspiro[fluorene-9,2′-[1,3]dithiolane]-6-carboxylate (0.62 g, 75.5% yield) as a white solid. LCMS (ESI) m/z=329 (M+H)⁺.

### Step 5: methyl 9,9-difluoro-7-methyl-9H-fluorene-3-carboxylate (6)

To a solution of NIS (2.83 g, 12.6 mmol) in DCM (8 mL) was added HF/Pyridine (6.5 mL, 65%) drop-wisely at 0° C. under N₂ atmosphere and the mixture was stirred at 0° C. under N₂ atmosphere for 30 minutes. To the reaction mixture was added a solution of methyl 2-methylspiro[fluorene-9,2′-[1,3]dithiolane]-6-carboxylate (0.59 g, 1.8 mmol) in DCM (6 mL) drop-wisely at 0° C. under N₂ atmosphere and the mixture was stirred at room temperature under N₂ atmosphere overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-4% EtOAc in PE) to give methyl 9,9-difluoro-7-methyl-9H-fluorene-3-carboxylate (423 mg, yield 85.7%) as a white solid.

### Step 6: 9,9-difluoro-7-methyl-9H-fluorene-3-carboxylic acid (7)

To a solution of methyl 9,9-difluoro-7-methyl-9H-fluorene-3-carboxylate (150 mg, 0.55 mmol) in MeOH (1 mL) and water (0.5 mL) was added LiOH·H₂O (92 mg, 2.2 mmol) at 0° C. and the mixture was stirred at room temperature for 16 hours. The mixture was acidified with 1 N aq·HCl solution to pH<3 and extracted with ethyl acetate twice. The combined organic layers were concentrated to dryness under reduced pressure to give 9,9-difluoro-7-methyl-9H-fluorene-3-carboxylic acid (120 mg, yield 83.8%) as a white solid, which was used directly in next step without further purification. LC/MS (ESI) (m/z): 261 (M+H)⁺.

### Step 7: methyl (9,9-difluoro-7-methyl-9H-fluorene-3-carbonyl)glycinate (8)

To a mixture of 9,9-difluoro-7-methyl-9H-fluorene-3-carboxylic acid (100 mg, 0.38 mmol) and methyl glycinate hydrochloride (97 mg, 0.76 mmol) in EtOAc (3 mL) was added HBTU (216 mg, 0.57 mmol) and DIPEA (156 mg, 1.2 mmol) under N₂ atmosphere at room temperature, the reaction mixture was stirred for 1.5 hours. The mixture was diluted with water and extracted with EtOAc twice. The combined organic layers were washed with brine, dried over anhydrous Na₂SO₄, filtered, and concentrated to dryness under reduced pressure. The residue was purified by column chromatography (silica gel, 0-30% EtOAc in PE) to give methyl (9,9-difluoro-7-methyl-9H-fluorene-3-carbonyl)glycinate (110 mg, yield 87.4%) as a yellow solid. LCMS (ESI) m/z=332 (M+H)⁺.

### Step 6: (9,9-difluoro-7-methyl-9H-fluorene-3-carbonyl)glycine (9)

To a solution of methyl (9,9-difluoro-7-methyl-9H-fluorene-3-carbonyl)glycinate (100 mg, 0.3 mmol) in MeOH (1 mL) and water (0.5 mL) was added LiOH·H₂O (42 mg, 1 mmol) at 0° C., the mixture was stirred at room temperature for 16 hours. The mixture was acidified with 1 N aq·HCl solution to pH<3 and extracted with ethyl acetate twice. The combined organic layers were concentrated to dryness under reduced pressure to give (9,9-difluoro-7-methyl-9H-fluorene-3-carbonyl)glycine (90 mg, yield 94.5%) as a white solid, which was used directly in next step without further purification. LC/MS (ESI) (m/z): 318(M+H)⁺.

### Step 9: methyl (S)-7-((9,9-difluoro-7-methyl-9H-fluorene-3-carbonyl)glycyl)-1,4-dioxa-7-azaspiro[4.4]nonane-6-carboxylate (10)

To a mixture of (9,9-difluoro-7-methyl-9H-fluorene-3-carbonyl)glycine (40 mg, 0.13 mmol) and methyl (S)-1,4-dioxa-7-azaspiro[4.4]nonane-8-carboxylate (38 mg, 0.2 mmol) in DMF (0.5 mL) was added DIPEA (52 mg, 0.4 mmol) and T₃P (127 mg, 0.2 mmol, 50% wt. in EtOAc) under N₂ atmosphere, the mixture was stirred at room temperature for 1 hour. The mixture was diluted with EtOAc, washed with saturated aq. Na₂CO₃ solution and brine, dried over anhydrous Na₂SO₄, filtered, and concentrated under reduced pressure to dryness. The residue was purified by flash chromatography (silica gel, 0-30% EtOAc in PE) to give methyl (S)-7-((9,9-difluoro-7-methyl-9H-fluorene-3-carbonyl)glycyl)-1,4-dioxa-7-azaspiro[4.4]nonane-8-carboxylate (45 mg, yield 71.2%) as a yellow solid. LC/MS (ESI) (m/z): 487 (M+H)⁺.

### Step 10: (S)-7-((9,9-difluoro-7-methyl-9H-fluorene-3-carbonyl)glycyl)-1,4-dioxa-7-azaspiro[4.4]nonane-8-carboxylic acid (11)

To a mixture of methyl (S)-7-((9,9-difluoro-7-methyl-9H-fluorene-3-carbonyl)glycyl)-1,4-dioxa-7-azaspiro [4.4]nonane-8-carboxylate (42 mg, 0.09 mmol) in MeOH (1 mL) and water (0.5 mL) was added LiOH·H₂O (21 mg, 0.5 mmol) at 0° C., the mixture was stirred at room temperature overnight. The mixture was acidified with 1 N aq·HCl to pH=3, extracted with EtOAc twice. The combined organic layers were washed with brine, dried over anhydrous Na₂SO₄, filtered, and concentrated to dryness under reduced pressure to give (S)-7-((9,9-difluoro-7-methyl-9H-fluorene-3-carbonyl)glycyl)-1,4-dioxa-7-azaspiro[4.4]nonane-8-carboxylic acid (35 mg, yield 82.3%) as a yellow solid, which was used in next step without purification LC/MS (ESI) m/z: 473 (M+H)⁺.

### Step 11: (S)—N—((R)-1-(4-carbamimidoylthiophen-2-yl)ethyl)-7-((9,9-difluoro-7-methyl-9H-fluorene-3-carbonyl)glycyl)-1,4-dioxa-7-azaspiro[4.4]nonane-6-carboxamide (Compound 190)

To a solution of (S)-7-((9,9-difluoro-7-methyl-9H-fluorene-3-carbonyl)glycyl)-1,4-dioxa-7-azaspiro[4.4]nonane-8-carboxylic acid (30 mg, 0.06 mmol) and (R)-5-(1-aminoethyl)thiophene-3-carboximidamide hydrochloride (19 mg, 0.09 mmol) in DMF (0.5 mL) was added DIPEA (26 mg, 0.2 mmol) and PyBOP (31 mg, 0.06 mmol) at 0° C. under N₂ atmosphere, the mixture was stirred at 25° C. for 1 hour. The reaction mixture was diluted with water and extracted with EtOAc twice. The combined organic layers were washed with brine, dried over anhydrous Na₂SO₄, filtered, and concentrated to dryness under reduced pressure. The residue was purified by prep-HPLC to give Compound 190 (3.2 mg, yield 8.1%) as a white solid. ¹H NMR (400 MHz, CD₃OD) δ 8.54 (s, 1H), 8.24 (d, J=12.9 Hz, 1H), 8.12 (s, 1H), 7.84 (d, J=7.9 Hz, 1H), 7.68 (d, J=7.9 Hz, 1H), 7.62 (d, J=7.8 Hz, 1H), 7.53 (d, J=10.9 Hz, 1H), 7.48 (s, 1H), 7.39 (d, J=7.8 Hz, 1H), 5.28 (d, J=7.1 Hz, 1H), 4.59-4.54 (m, 1H), 4.20 (q, J=16.6 Hz, 2H), 3.99 (dd, J=12.6, 8.3 Hz, 4H), 3.80 (q, J=11.0 Hz, 2H), 2.48-2.39 (m, 4H), 2.23-2.18 (m, 1H), 1.58 (d, J=6.9 Hz, 3H).LCMS (ESI) (m/z): 624 (M+H)⁺.

### Scheme 182: Synthesis of (S)—N—((R)-1-(4-carbamimidoylthiophen-2-yl)ethyl)-7-((9,9-difluoro-7-methoxy-9H-fluorene-3-carbonyl)glycyl)-1,4-dioxa-7-azaspiro[4.4]nonane-8-carboxamide (Compound 191)

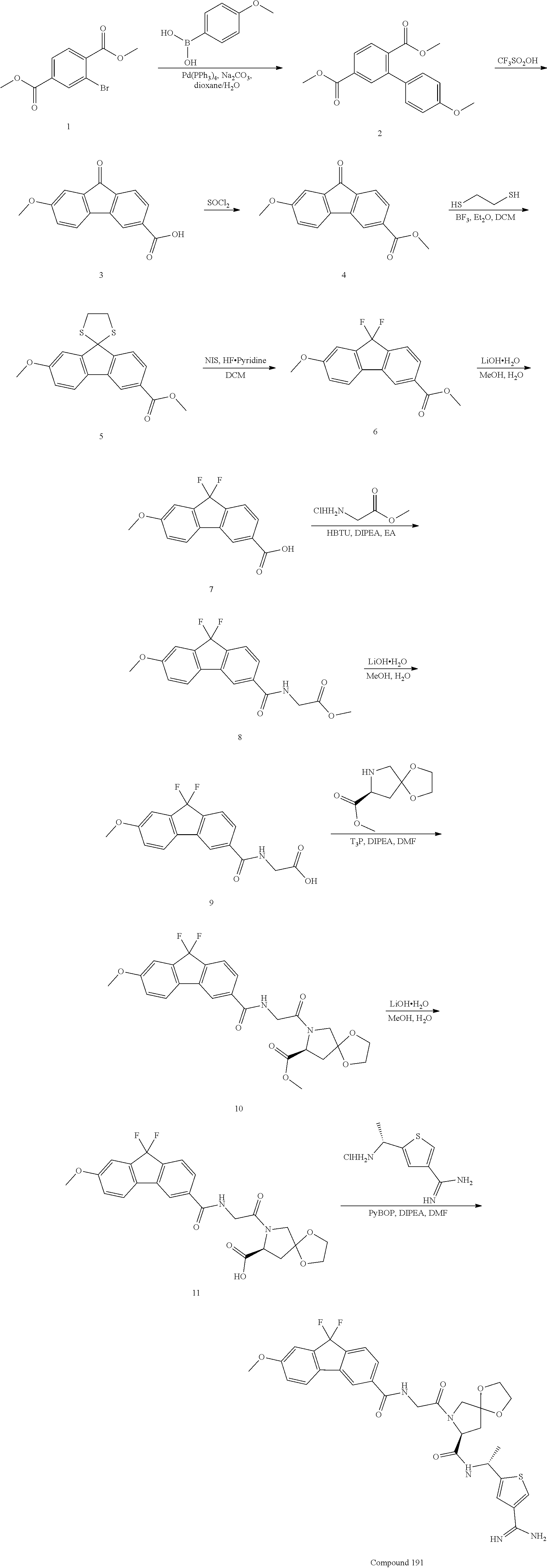

Compound 191 was prepared as a white solid from dimethyl 2-bromoterephthalate, (4-methoxyphenyl)boronic acid, methyl glycinate hydrochloride, methyl (S)-1,4-dioxa-7-azaspiro[4.4]nonane-8-carboxylate, and (R)-5-(1-aminoethyl)thiophene-3-carboximidamide hydrochloride based on the procedures set forth in Scheme 181. ¹H NMR (400 MHz, CD₃OD) δ 8.50 (s, 1H), 8.24 (d, J=14.7 Hz, 1H), 8.06 (s, 1H), 7.79 (d, J=7.8 Hz, 1H), 7.70-7.60 (m, 2H), 7.53 (d, J=10.7 Hz, 1H), 7.22 (s, 1H), 7.12 (d, J=8.4 Hz, 1H), 5.29 (q, J=6.8 Hz, 1H), 4.60-4.55 (m, 1H), 4.27-4.11 (m, 2H), 4.05-3.96 (m, 4H), 3.89 (s, 3H), 3.80 (q, J=10.7 Hz, 2H), 2.45 (dd, J=13.1, 8.9 Hz, 1H), 2.22 (dd, J=13.0, 6.7 Hz, 1H), 1.58 (d, J=6.9 Hz, 3H). LCMS (ESI) (m/z): 640 (M+H)⁺.

### Scheme 183: Synthesis of (8S)—N-[(1R)-1-(4-carbamimidoylthiophen-2-yl)ethyl]-7-{2-[(7-chloro-9,9-difluorofluoren-3-yl)formamido]acetyl}-1,4-dioxa-7-azaspiro[4.4]nonane-8-carboxamide (Compound 192)

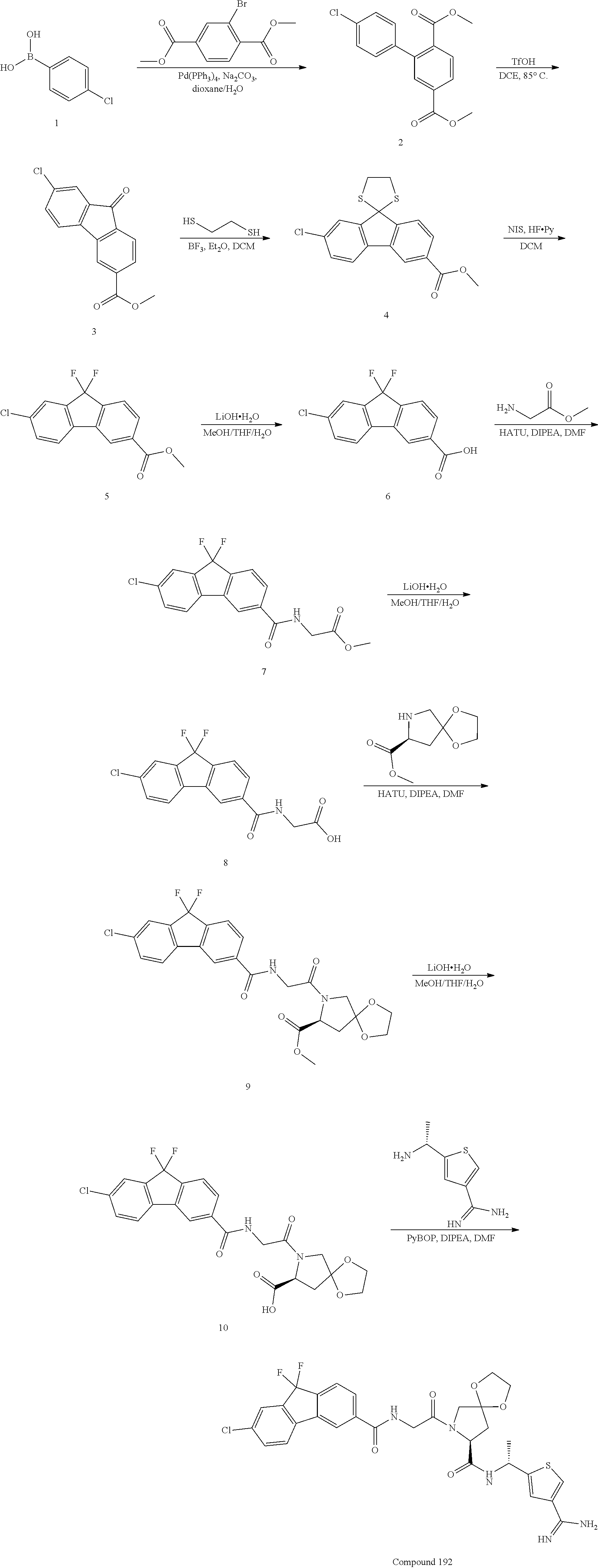

Compound 192 was prepared from (4-chlorophenyl)boronic acid, 1,4-dimethyl 2-bromobenzene-1,4-dicarboxylate, methyl 2-aminoacetate hydrochloride, methyl (8S)-1,4-dioxa-7-azaspiro[4.4]nonane-8-carboxylate, and (5-[(1R)-1-aminoethyl]thiophene-3-carboximidamide based on the procedures set forth in Scheme 173. ¹H NMR (400 MHz, CD₃OD) δ 8.28-8.13 (m, 2H), 7.90 (d, J=7.6 Hz, 1H), 7.80-7.71 (m, 2H), 7.69 (s, 1H), 7.60 (d, J=8.2 Hz, 1H), 7.52 (d, J=10.8 Hz, 1H), 5.40-5.23 (m, 1H), 4.61-4.52 (m, 1H), 4.28-4.11 (m, 2H), 4.04-3.95 (m, 4H), 3.85-3.74 (m, 2H), 2.62-2.39 (m, 1H), 2.36-2.18 (m, 1H), 1.68-1.55 (m, 3H). LC/MS (ESI) m/z: 644 (M+H)⁺.

### Scheme 184: Synthesis of (8S)—N-[(1R)-1-(4-carbamimidoylthiophen-2-yl)ethyl]-7-{2-[(7,9,9-trifluorofluoren-3-yl)formamido]acetyl}-1,4-dioxa-7-azaspiro[4.4]nonane-8-carboxamide (Compound 193)

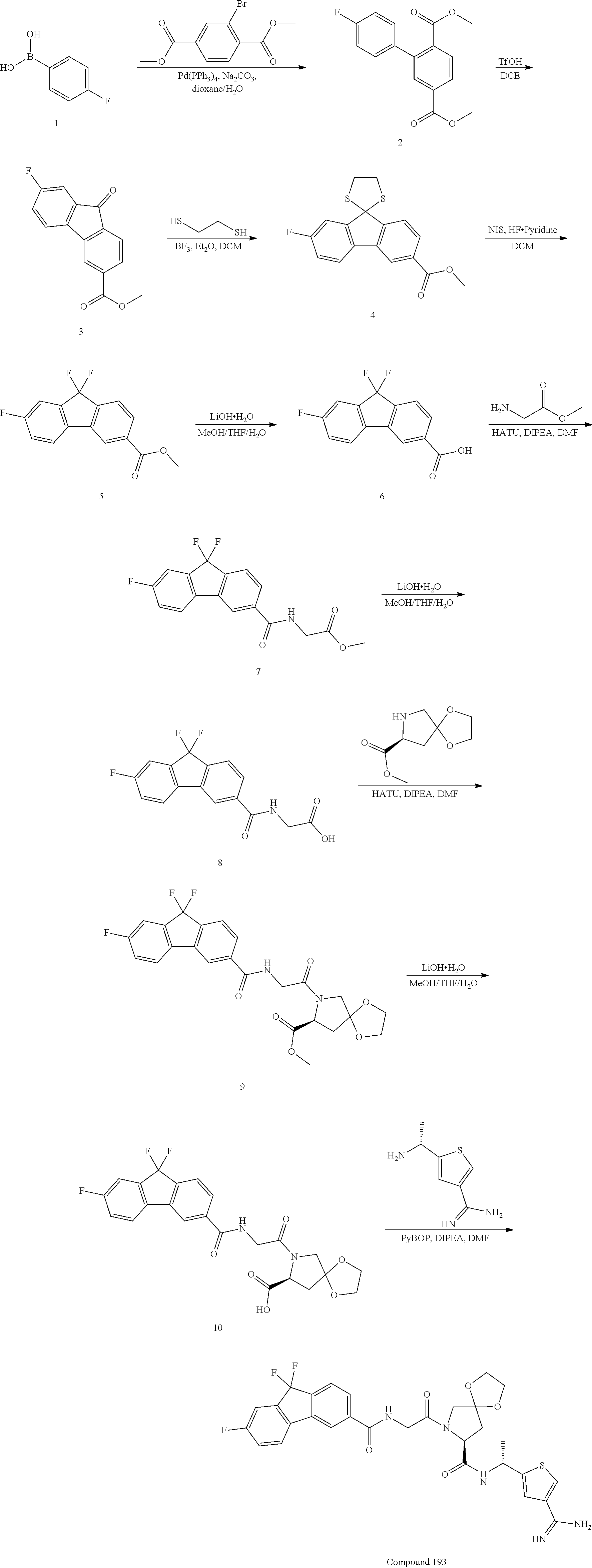

Compound 193 was prepared from 4-fluorophenylboronic acid, 1,4-dimethyl 2-bromobenzene-1,4-dicarboxylate, methyl 2-aminoacetate hydrochloride, methyl (8S)-1,4-dioxa-7-azaspiro[4.4]nonane-8-carboxylate, and (5-[(1R)-1-aminoethyl]thiophene-3-carboximidamide based on the procedures set forth in Scheme 173. ¹H NMR (400 MHz, CD₃OD) δ 8.26-8.19 (m, 1H), 8.16 (s, 1H), 7.87 (d, J=7.6 Hz, 1H), 7.80-7.75 (m, 1H), 7.72 (d, J=7.2 Hz, 1H), 7.52 (d, J=10.8 Hz, 1H), 7.45 (d, J=8.0 Hz, 1H), 7.33 (t, J=8.6 Hz, 1H), 5.40-5.25 (m, 1H), 4.59-4.54 (m, 1H), 4.26-4.15 (m, 2H), 4.02-3.94 (m, 4H), 3.84-3.76 (m, 2H), 2.50-2.40 (m, 1H), 2.26-2.17 (m, 1H), 1.58 (d, J=7.2 Hz, 3H). LC/MS (ESI) m/z: 628 (M+H)⁺.

### Scheme 185: Synthesis of 10-(((3R,5S)-5-(((R)-1-(4-carbamimidoylthiophen-2-yl)ethyl) carbamoyl)-1-((9,9-difluoro-9H-fluorene-3-carbonyl)glycyl)-3-fluoropyrrolidin-3-yl)methoxy) decanoic acid (Compound 194)

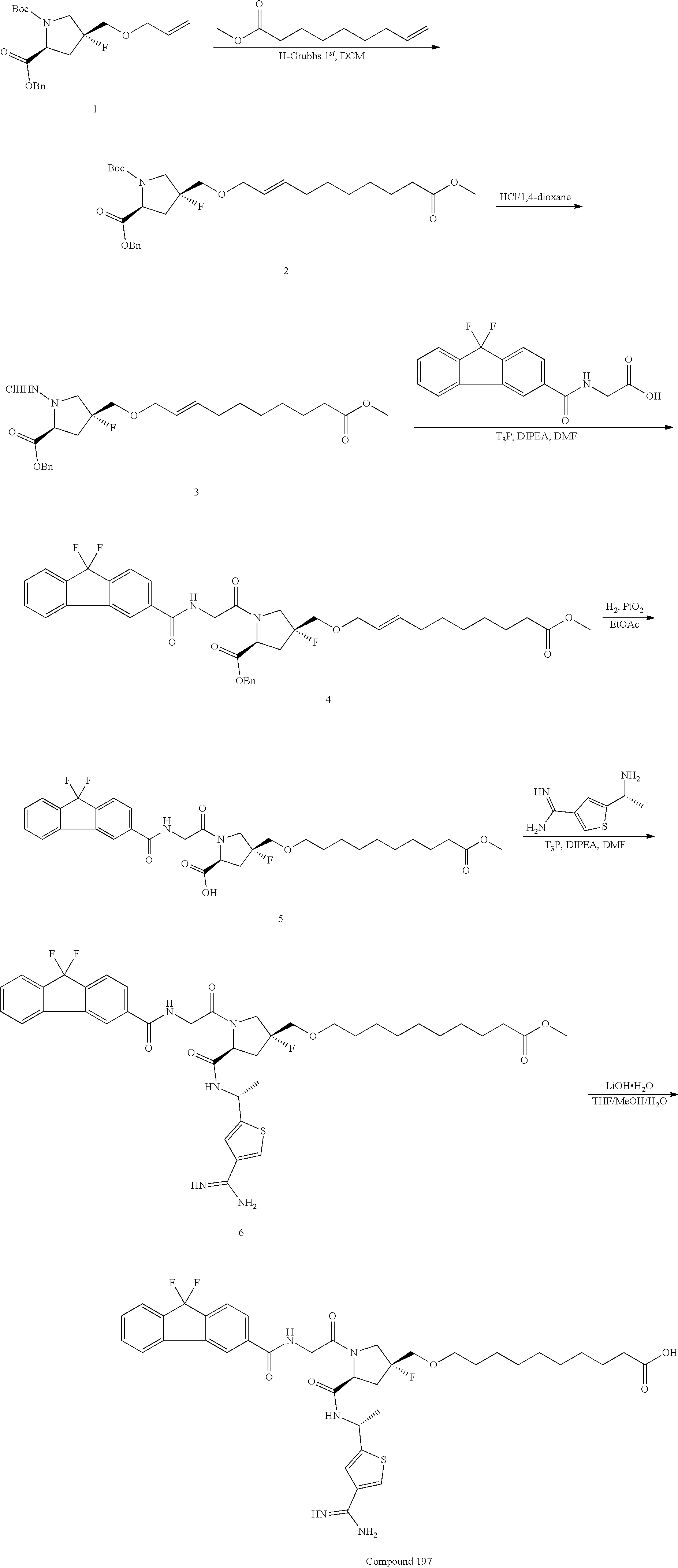

### Step 1: 2-benzyl 1-(tert-butyl) (2S,4R)-4-fluoro-4-((((E)-10-methoxy-10-oxodec-2-en-1-yl)oxy)methyl) pyrrolidine-1,2-dicarboxylate (2)

To a mixture of 2-benzyl 1-(tert-butyl) (2S,4R)-4-((allyloxy)methyl)-4-fluoropyrrolidine-1,2-dicarboxylate (200 mg, 0.5 mmol) and methyl non-8-enoate (170 mg, 1.0 mmol) in dry DCM (2 mL) was added Hoveyda-Grubbs 1^{st} (80 mg, 0.13 mmol) under N₂ atmosphere and the mixture was stirred at 35° C. for 18 hours under N₂ atmosphere. The mixture was concentrated to dryness under reduced pressure and the residue was purified by flash chromatography (silica gel, 0-30% EtOAc in PE) to give 2-benzyl 1-(tert-butyl) (2S,4R)-4-fluoro-4-((((E)-10-methoxy-10-oxodec-2-en-1-yl)oxy)methyl)pyrrolidine-1,2-dicarboxylate (138 mg, yield 51.5%) as a brown oil. LC/MS (ESI) m/z: 536 (M+H)⁺.

### Step 2: benzyl (2S,4R)-4-fluoro-4-((((E)-10-methoxy-10-oxodec-2-en-1-yl)oxy)methyl)pyrrolidine-2-carboxylate hydrochloride (3)

To a solution of 2-benzyl 1-(tert-butyl) (2S,4R)-4-fluoro-4-((((E)-10-methoxy-10-oxodec-2-en-1-yl)oxy)methyl)pyrrolidine-1,2-dicarboxylate (138 mg, 0.26 mmol) in DCM (1.5 mL) was added HCl/1,4-dioxane (1 mL) and the mixture was stirred at room temperature for 2 hours. The reaction mixture was concentrated to dryness under reduced pressure to give benzyl (2S,4R)-4-fluoro-4-((((E)-10-methoxy-10-oxodec-2-en-1-yl)oxy)methyl)pyrrolidine-2-carboxylate hydrochloride (112 mg, crude) as a white solid, which was used directly in the next step without further purification. LC/MS (ESI) m/z: 436 (M+H)⁺.

### Step 3: benzyl (2S,4R)-1-((9,9-difluoro-9H-fluorene-3-carbonyl)glycyl)-4-fluoro-4-((((E)-10-methoxy-10-oxodec-2-en-1-yl)oxy)methyl)pyrrolidine-2-carboxylate (4)

To a mixture of benzyl (2S,4R)-4-fluoro-4-((((E)-10-methoxy-10-oxodec-2-en-1-yl)oxy)methyl)pyrrolidine-2-carboxylate hydrochloride (112 mg, 0.26 mmol) and (9,9-difluoro-9H-fluorene-3-carbonyl)glycine (78 mg, 0.26 mmol) in DMF (2 mL) was added DIPEA (168 mg, 1.3 mmol) and T₃P (248 mg, 0.39 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-10% MeOH in DCM) to give benzyl (2S,4R)-1-((9,9-difluoro-9H-fluorene-3-carbonyl)glycyl)-4-fluoro-4-((((E)-10-methoxy-10-oxodec-2-en-1-yl)oxy)methyl)pyrrolidine-2-carboxylate (60 mg, yield 30.1%) as a colorless oil. LC/MS (ESI) m/z: 721 (M+H)⁺.

### Step 4: (2S,4R)-1-((9,9-difluoro-9H-fluorene-3-carbonyl)glycyl)-4-fluoro-4-(((10-methoxy-10-oxodecyl) oxy)methyl)pyrrolidine-2-carboxylic acid (5)

To a solution of benzyl (2S,4R)-1-((9,9-difluoro-9H-fluorene-3-carbonyl)glycyl)-4-fluoro-4-((((E)-10-methoxy-10-oxodec-2-en-1-yl)oxy)methyl)pyrrolidine-2-carboxylate (60 mg, 0.083 mmol) in EtOAc (2 mL) was added PtO₂ (10 mg, 0.042 mmol) and the mixture was degassed under N₂ atmosphere for three times and stirred under a H₂ balloon at room temperature for 30 mins. The mixture was filtered, and the filtrate was concentrated to dryness under reduced pressure to give (2S,4R)-1-((9,9-difluoro-9H-fluorene-3-carbonyl)glycyl)-4-fluoro-4-(((10-methoxy-10-oxodecyl)oxy)methyl)pyrrolidine-2-carboxylic acid (44 mg, yield 83.8%) as a colorless oil, which was used directly in the next step. LC/MS (ESI) (m/z): 633 (M+H)⁺.

### Step 5: methyl 10-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)decanoate (6)

To a mixture of (2S,4R)-1-((9,9-difluoro-9H-fluorene-3-carbonyl)glycyl)-4-fluoro-4-(((10-methoxy-10-oxodecyl)oxy)methyl)pyrrolidine-2-carboxylic acid (44 mg, 0.07 mmol) and (R)-5-(1-aminoethyl) thiophene-3-carboximidamide hydrochloride (17 mg, 0.084 mmol) in DMF (1 mL) was added DIPEA (45 mg, 0.35 mmol) and PyBOP (40 mg, 0.077 mmol) at 0° C. and the mixture was stirred under N₂ atmosphere at room temperature for 1 hour. The reaction mixture was diluted with water and extracted with EtOAc twice. The combined organic layers were washed with brine, dried over anhydrous Na₂SO₄, filtered, and concentrated to dryness under reduced pressure. The residue was purified by prep-TLC (DCM:MeOH=10:1) to give methyl 10-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)decanoate (23 mg, yield 41.9%) as a white solid. LCMS (ESI) (m/z): 784 (M+H)⁺.

### Step 6: 10-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)decanoic acid (Compound 194)

To a solution of methyl 10-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)decanoate (22 mg, 0.028 mmol) in MeOH (0.75 mL) and water (0.25 mL) was added LiOH·H₂O (3 mg, 0.056 mmol) at 0° C. and the mixture was stirred at room temperature for 3 hours. The mixture was acidified with 1 N aq·HCl to pH˜4 and concentrated to dryness under reduced pressure. The residue was purified by prep-HPLC to give Compound 194 (2.1 mg, yield 9.7%) as a white solid. ¹H NMR (400 MHz, CD₃OD) δ 8.55-8.49 (m, 1H), 8.27-8.16 (m, 2H), 7.87 (t, J=8.8 Hz, 1H), 7.74 (dd, J=17.5, 7.5 Hz, 2H), 7.66 (d, J=7.8 Hz, 1H), 7.57 (dd, J=14.4, 6.5 Hz, 2H), 7.45 (t, J=7.5 Hz, 1H), 5.28 (q, J=6.8 Hz, 1H), 4.62 (dd, J=19.3, 10.6 Hz, 1H), 4.25 (dd, J=50.0, 16.7 Hz, 2H), 4.08-3.88 (m, 2H), 3.75-3.67 (m, 2H), 3.53 (t, J=6.4 Hz, 2H), 2.63-2.44 (m, 1H), 2.32-2.23 (m, 1H), 2.21 (d, J=7.4 Hz, 2H), 1.70-1.54 (m, 8H), 1.31 (s, 9H). LCMS (ESI) (m/z): 770 (M+H)⁺.

### Scheme 186: (S)-7-((4-(1H-pyrrolo[2,3-b]pyridin-5-yl)benzoyl)glycyl)-N-((4-carbamimidoylthiophen-2-yl)methyl)-1,4-dioxa-7-azaspiro[4.4]nonane-8-carboxamide (Compound 195)

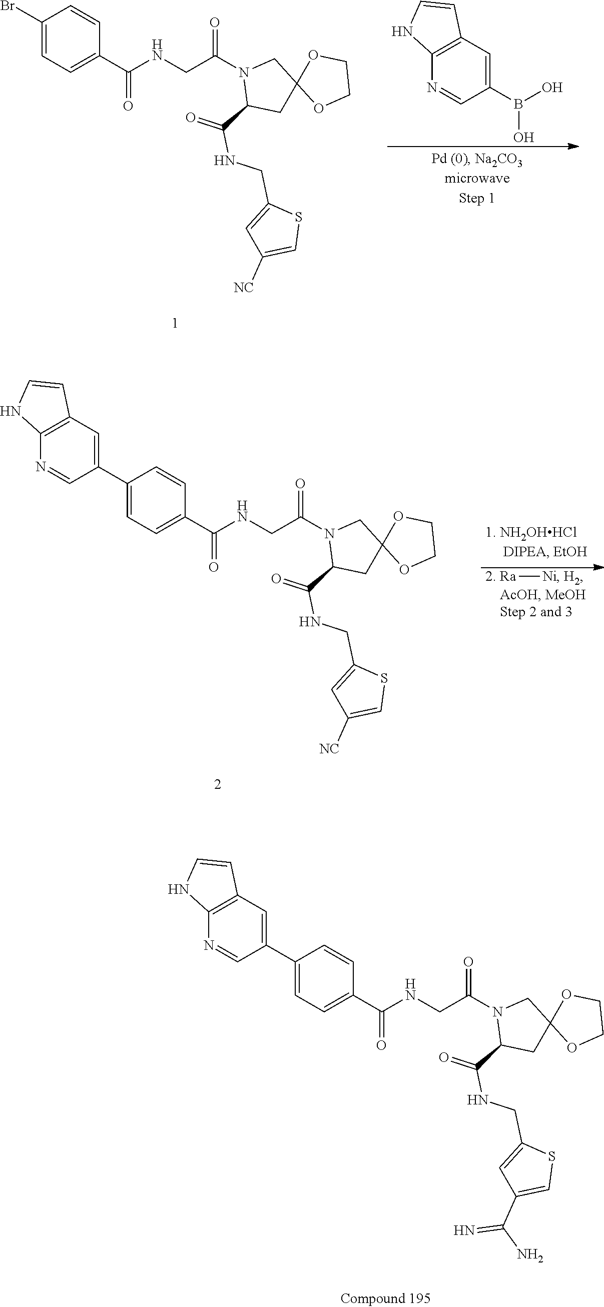

Compound 195 was prepared from 1H-pyrrolo[2,3-b]pyridin-5-ylboronic acid, (8S)-7-[2-[(4-bromobenzoyl)amino]acetyl]-N-[(5-cyano-3-thienyl)methyl]-1,4-dioxa-7-azaspiro[4.4]nonane-8-carboxamide, and NH₂OH·HCl based on the procedures set forth in Scheme 103 LC/MS (ESI) m/z: 588 (M+H)⁺. ¹H NMR (400 MHz, DMSO-d₆) δ 11.75 (s, 1H), 8.98 (s, 1H), 8.71 (t, J=5.7 Hz, 1H), 8.47-8.16 (m, 1H), 7.97 (d, J=8.1 Hz, 2H), 7.84 (d, J=8.2 Hz, 2H), 7.60-7.23 (m, 2H), 6.53 (d, J=3.5 Hz, 2H), 4.53 (d, J=5.7 Hz, 1H), 4.47-4.28 (m, 2H), 4.28-4.10 (m, 2H), 4.06-3.85 (m, 2H), 3.84-3.71 (m, 2H), 3.63 (d, J=10.8 Hz, 2H), 2.39-2.28 (m, 1H), 2.13-2.00 (m, 1H).

### Scheme 187: (S)—N-((4-carbamimidoylthiophen-2-yl)methyl)-7-((2′,6′-difluoro-[1,1′-biphenyl]-4-carbonyl)glycyl)-1,4-dioxa-7-azaspiro[4.4]nonane-8-carboxamide (Compound 196)

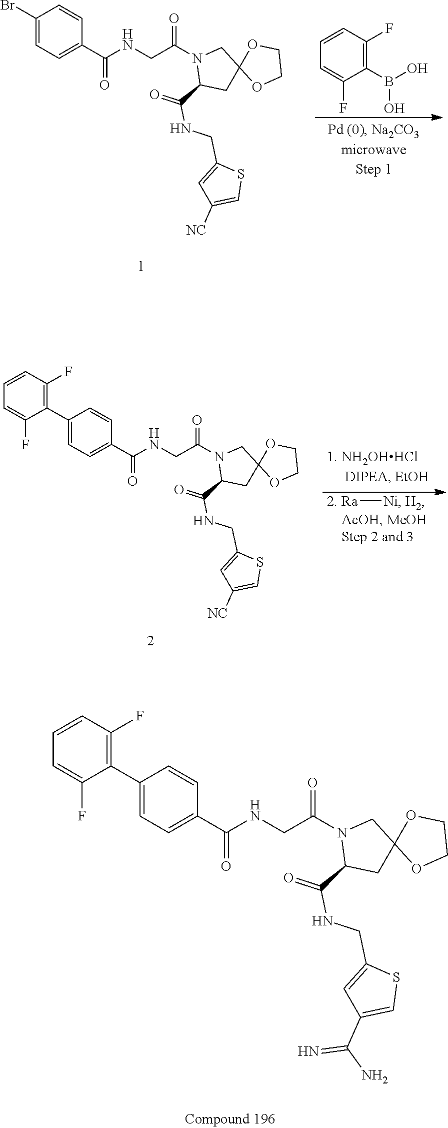

Compound 196 was prepared from (2,6-difluorophenyl)boronic acid, (8S)-7-[2-[(4-bromobenzoyl)amino]acetyl]-N-[(5-cyano-3-thienyl)methyl]-1,4-dioxa-7-azaspiro[4.4]nonane-8-carboxamide, and NH₂OH·HCl based on the procedures set forth in Scheme 103. LC/MS (ESI) m/z: 584 (M+H)+. ¹H NMR (400 MHz, DMSO-d₆) δ 8.96 (s, 1H), 8.75 (q, J=7.9, 6.9 Hz, 1H), 8.54 (t, J=5.8 Hz, 1H), 8.42 (s, 1H), 8.33 (d, J=9.5 Hz, 1H), 7.65 (d, J=7.7 Hz, 1H), 7.56-7.37 (m, 3H), 7.24-7.13 (m, 2H), 7.04 (d, J=8.0 Hz, 2H), 4.63 (td, J=13.2, 12.3, 4.4 Hz, 2H), 4.49-4.29 (m, 4H), 4.02 (dd, J=16.4, 5.9 Hz, 2H), 3.85-3.70 (m, 3H), 2.28 (t, J=12.3 Hz, 1H), 1.98 (dd, J=13.2, 3.0 Hz, 1H).

### Scheme 188: (S)—N-((4-carbamimidoylthiophen-2-yl)methyl)-7-((2′,6′-dimethyl-[1,1′-biphenyl]-4-carbonyl)glycyl)-1,4-dioxa-7-azaspiro[4.4]nonane-8-carboxamide (Compound 197)

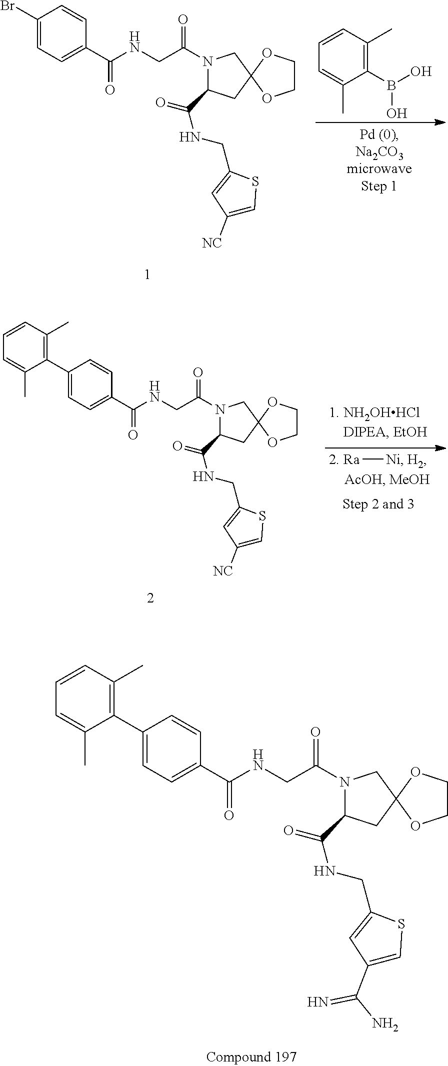

Compound 197 was prepared from (2,6-dimethylphenyl)boronic acid, (8S)-7-[2-[(4-bromobenzoyl)amino]acetyl]-N-[(5-cyano-3-thienyl)methyl]-1,4-dioxa-7-azaspiro[4.4]nonane-8-carboxamide, and NH₂OH·HCl based on the procedures set forth in Scheme 103. LC/MS (ESI) m/z:576 (M+H)⁺. ¹H NMR (400 MHz, DMSO-d₆) 8.71 (d, J=6.9 Hz, 1H), 8.64 (s, 1H), 8.41 (s, 1H), 7.92 (d, J=7.9 Hz, 2H), 7.81-7.69 (m, 2H), 7.40 (d, J=7.8 Hz, 2H), 7.10 (dd, J=14.9, 8.0 Hz, 2H), 4.56-4.48 (m, 3H), 4.40 (t, J=7.8 Hz, 2H), 4.19 (dd, J=16.8, 5.5 Hz, 1H), 3.95-3.85 (m, J=17.4, 6.5 Hz, 4H), 3.81 (d, J=10.8 Hz, 1H), 3.74-3.60 (m, 1H), 2.34 (s, 3H), 2.20 (s, 3H), 2.07-2.0 (m, 2H).

### Scheme 189: (S)-7-((4-(1-benzyl-1H-pyrazol-4-yl)benzoyl)glycyl)-N-((4-carbamimidoylthiophen-2-yl)methyl)-1,4-dioxa-7-azaspiro[4.4]nonane-8-carboxamide (Compound 198)

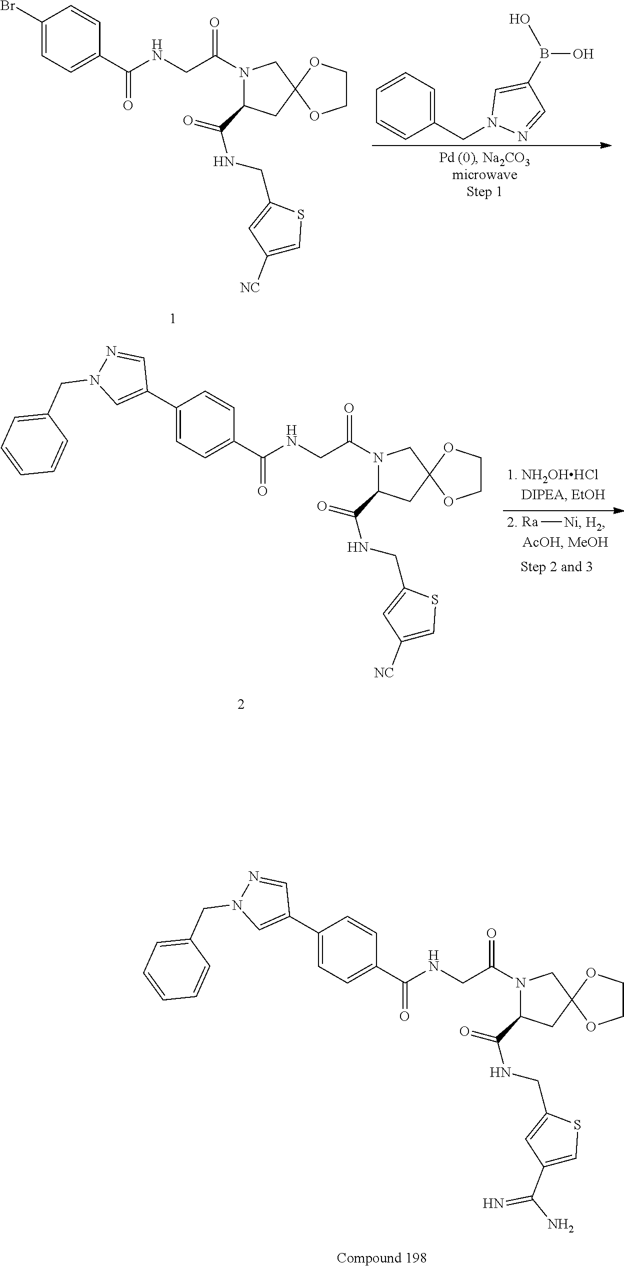

Compound 198 was prepared from (1-benzylpyrazol-4-yl)boronic acid, (8S)-7-[2-[(4-bromobenzoyl)amino]acetyl]-N-[(5-cyano-3-thienyl)methyl]-1,4-dioxa-7-azaspiro[4.4]nonane-8-carboxamide, and NH₂OH·HCl based on the procedures set forth in Scheme 103. LC/MS (ESI) m/z: 628 (M+H)⁺. ¹H NMR (400 MHz, DMSO-d₆) δ 9.01 (d, J=6.1 Hz, 1H), 8.64 (dt, J=12.6, 5.7 Hz, 2H), 8.52-8.32 (m, 3H), 8.03 (s, 1H), 7.88 (t, J=8.0 Hz, 2H), 7.70 (d, J=8.0 Hz, 2H), 7.58-7.46 (m, 1H), 7.42-7.25 (m, 2H), 4.83-4.68 (m, 1H), 4.72-4.28 (m, 2H), 4.16 (dt, J=14.0, 6.9 Hz, 1H), 4.03-3.75 (m, 4H), 2.53 (t, J=2.0 Hz, 4H), 2.42-2.17 (m, 1H), 2.07 (dd, J=13.1, 6.8 Hz, 1H).

### Scheme 190: (S)—N-((4-carbamimidoylthiophen-2-yl)methyl)-7-((4′-fluoro-3′-methyl-[1,1′-biphenyl]-4-carbonyl)glycyl)-1,4-dioxa-7-azaspiro[4.4]nonane-8-carboxamide (Compound 199)

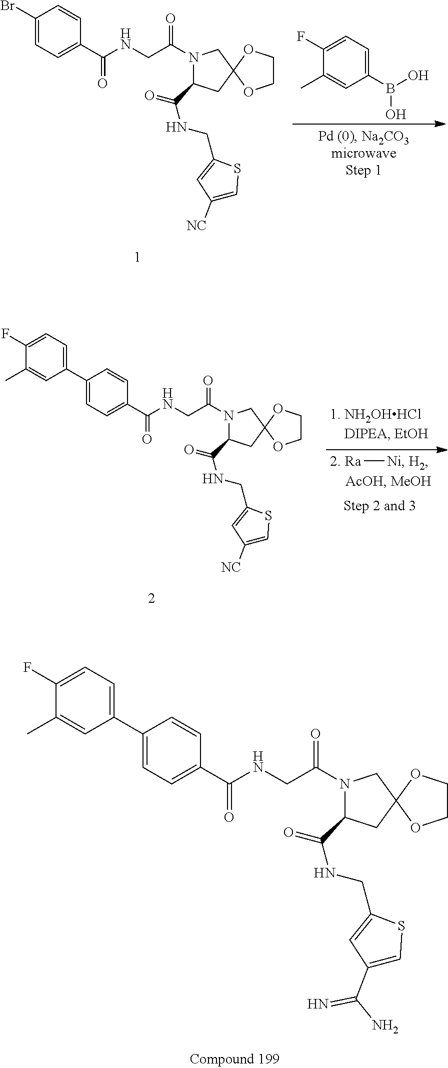

Compound 199 was prepared from (4-fluoro-3-methyl-phenyl)boronic acid (15.59 mg, 0.1013 mmol, 1.2 equiv.), (8S)-7-[2-[(4-bromobenzoyl)amino]acetyl]-N-[(5-cyano-3-thienyl)methyl]-1,4-dioxa-7-azaspiro[4.4]nonane-8-carboxamide, and NH₂OH·HCl based on the procedures set forth in Scheme 103 LC/MS (ESI) m/z: 580 (M+H)⁺. ¹H NMR (400 MHz, DMSO-d₆) δ 8.92 (s, 1H), 8.65 (s, 1H), 8.53-8.39 (m, 1H), 8.24 (s, 1H), 7.87 (d, J=7.8 Hz, 2H), 7.69 (d, J=7.9 Hz, 2H), 7.62 (d, J=7.3 Hz, 2H), 7.52-7.40 (m, 2H), 7.17 (t, J=9.1 Hz, 2H), 4.54-4.19 (m, 2H), 4.11 (d, J=14.2 Hz, 2H), 4.02-3.66 (m, 2H), 3.69-3.41 (m, 3H), 3.10-2.99 (m, 2H), 2.25 (s, 3H), 2.08-2.02 (m, 1H), 1.98 (dd, J=12.5, 6.7 Hz, 1H).

### Scheme 191: Synthesis of (S)—N—((R)-1-(4-carbamoylthiophen-2-yl)ethyl)-7-((9,9-difluoro-9H-fluorene-3-carbonyl)glycyl)-1,4-dioxa-7-azaspiro[4.4]nonane-8-carboxamide (Compound 200)

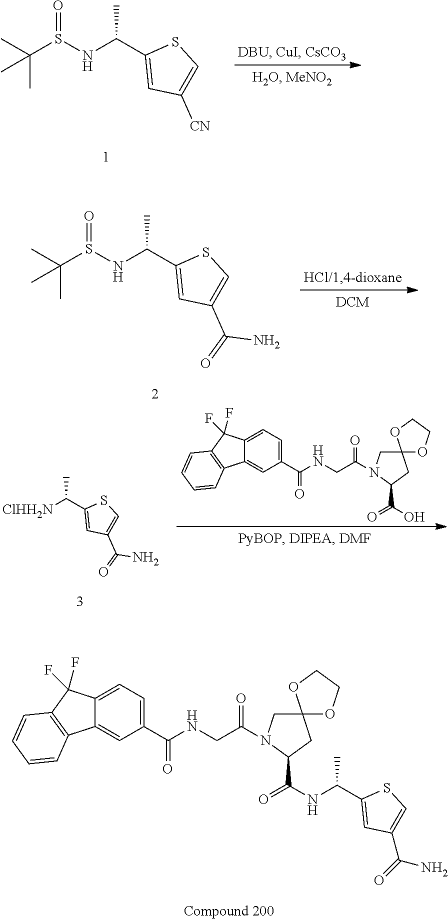

### Step 1: 5-((1R)-1-((tert-butylsulfinyl)amino)ethyl)thiophene-3-carboxamide (2)

To a solution of N—((R)-1-(4-cyanothiophen-2-yl)ethyl)-2-methylpropane-2-sulfinamide (125 mg, 0.5 mmol) in MeNO₂ (1 mL) and water (1 mL) was added DBU (152 mg, 1 mmol), CuI (19 mg, 0.1 mmol) and CSCO₃ (81 mg, 0.25 mmol) at room temperature and the mixture was stirred at 100° C. for 1 hour. The mixture was diluted with water and extracted with EtOAc twice. The combined organic layers were washed with brine, dried over anhydrous Na₂SO₄, filtered, and concentrated under reduced pressure to dryness. The residue was purified by column chromatography on (silica gel, 0-10% MeOH in DCM) to give 5-((1R)-1-((tert-butylsulfinyl)amino)ethyl)thiophene-3-carboxamide (80 mg, yield 58.2%) as a yellow oil. LCMS (ESI) m/z: 275 (M+H)⁺.

### Step 2: (R)-5-(1-aminoethyl)thiophene-3-carboxamide hydrochloride (3)

To a solution of 5-((1R)-1-((tert-butylsulfinyl)amino)ethyl)thiophene-3-carboxamide (80 mg, 0.29 mmol) in DCM (1 mL) was added HCl/1,4-dioxane (1 mL, 4M) and the mixture was stirred under N₂ atmosphere at room temperature for 2 hours. The reaction mixture was concentrated to dryness under reduced pressure to give (R)-5-(1-aminoethyl)thiophene-3-carboxamide hydrochloride (76 mg, crude) as a white solid, which was used directly in the next step without further purification. LC/MS (ESI) m/z: 171 (M+H)⁺.

### Step 3: (S)—N—((R)-1-(4-carbamoylthiophen-2-yl)ethyl)-7-((9,9-difluoro-9H-fluorene-3-carbonyl)glycyl)-1,4-dioxa-7-azaspiro[4.4]nonane-8-carboxamide (Compound 200)

To a mixture of (S)-7-((9,9-difluoro-9H-fluorene-3-carbonyl)glycyl)-1,4-dioxa-7-azaspiro[4.4]nonane-8-carboxylic acid (142 mg, 0.31 mmol) and (R)-5-(1-aminoethyl)thiophene-3-carboxamide hydrochloride (76 mg, 0.37 mmol) in DMF (2 mL) was added DIPEA (200 mg, 1.55 mmol) and PyBOP (177 mg, 0.34 mmol) at room temperature and the mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and prep-HPLC to give Compound 200 (10.0 mg, yield 5.3%) as a white solid. ¹H NMR (400 MHz, CD₃OD) δ 8.16 (s, 1H), 8.01-7.81 (m, 2H), 7.80-7.67 (m, 2H), 7.64 (d, J=6.9 Hz, 1H), 7.57 (t, J=7.3 Hz, 1H), 7.44 (dd, J=18.1, 10.6 Hz, 2H), 5.27 (q, J=6.7 Hz, 1H), 4.59 (dd, J=8.9, 6.0 Hz, 1H), 4.31-4.09 (m, 2H), 3.98 (d, J=16.0 Hz, 4H), 3.79 (s, 2H), 2.43 (dd, J=13.1, 9.1 Hz, 1H), 2.22 (dd, J=13.2, 6.0 Hz, 1H), 1.59 (dd, J=37.0, 6.9 Hz, 3H). LC/MS (ESI) m/z: 611 (M+H)⁺.

### Scheme 192: Synthesis of 6-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)hexanoic acid (Compound 201)

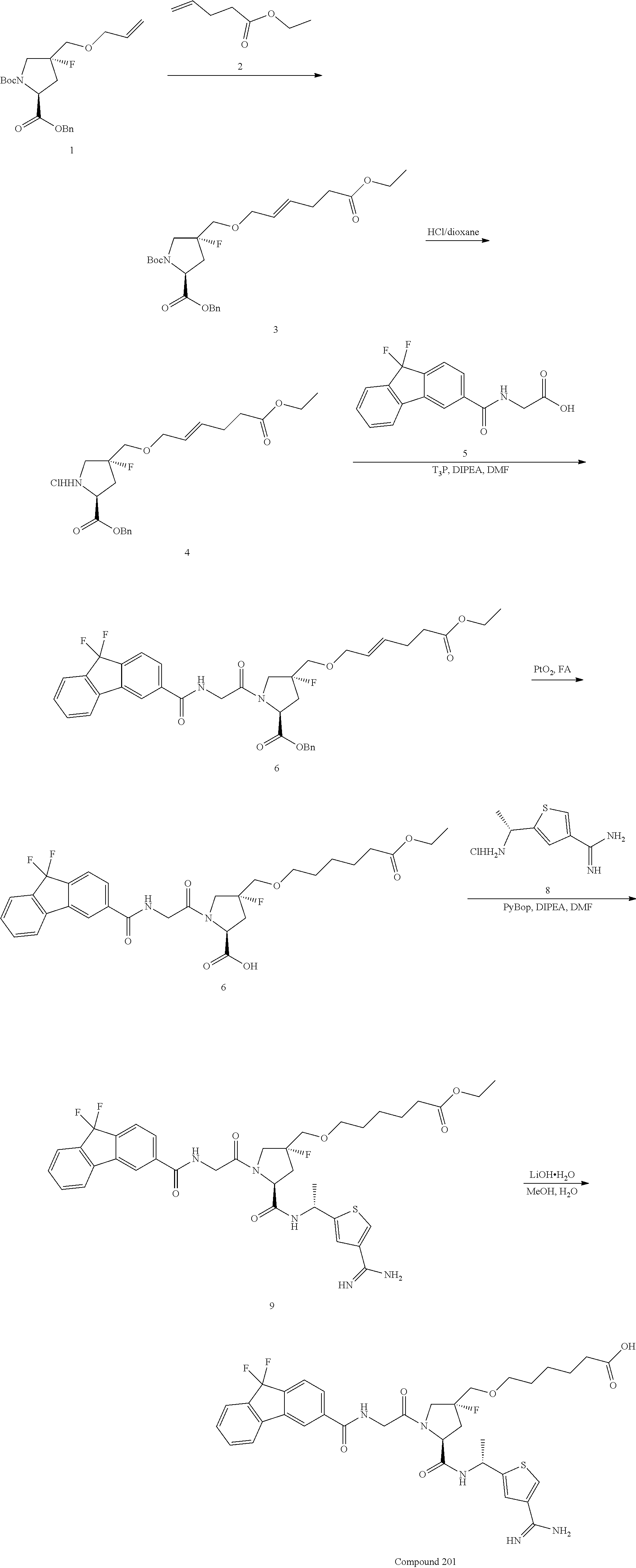

### Step 1: 2-benzyl 1-(tert-butyl) (2S,4R)-4-((((E)-6-ethoxy-6-oxohex-2-en-1-yl)oxy)methyl)-4-fluoropyrrolidine-1,2-dicarboxylate (3)

To a mixture of 2-benzyl 1-(tert-butyl) (2S,4R)-4-((allyloxy)methyl)-4-fluoropyrrolidine-1,2-dicarboxylate (180 mg, 0.46 mmol) and ethyl pent-4-enoate (118 mg, 0.92 mmol) in dry DCM (2 mL) was added Hoveyda-Grubbs 1^{st} (80 mg, 0.13 mmol) under N₂ atmosphere and the mixture was stirred at 35° C. for 18 hours under N₂ atmosphere. The mixture was concentrated to dryness under reduced pressure and the residue was purified by flash chromatography (silica gel, 0-30% EtOAc in PE) to give 2-benzyl 1-(tert-butyl) (2S,4R)-4-((((E)-6-ethoxy-6-oxohex-2-en-1-yl)oxy)methyl)-4-fluoropyrrolidine-1,2-dicarboxylate (110 mg, yield 48.4%) as a yellow oil. LC/MS (ESI) m/z: 494 (M+H)⁺.

### Step 2: benzyl (2S,4R)-4-((((E)-6-ethoxy-6-oxohex-2-en-1-yl)oxy)methyl)-4-fluoropyrrolidine-2-carboxylate hydrochloride (4)

To a solution of 2-benzyl 1-(tert-butyl) (2S,4R)-4-((((E)-6-ethoxy-6-oxohex-2-en-1-yl)oxy)methyl)-4-fluoropyrrolidine-1,2-dicarboxylate (100 mg, 0.20 mmol) in DCM (2.0 mL) was added HCl/1,4-dioxane (1 mL) and the mixture was stirred at room temperature for 2 hours. The reaction mixture was concentrated to dryness under reduced pressure to give benzyl (2S,4R)-4-((((E)-6-ethoxy-6-oxohex-2-en-1-yl)oxy)methyl)-4-fluoropyrrolidine-2-carboxylate hydrochloride (87 mg, crude) as a white solid, which was used directly in the next step without further purification. LC/MS (ESI) m/z: 394 (M+H)⁺.

### Step 3: benzyl (2S,4R)-1-((9,9-difluoro-9H-fluorene-3-carbonyl)glycyl)-4-((((E)-6-ethoxy-6-oxohex-2-en-1-yl)oxy)methyl)-4-fluoropyrrolidine-2-carboxylate (6)

To a mixture of benzyl (2S,4R)-4-((((E)-6-ethoxy-6-oxohex-2-en-1-yl)oxy)methyl)-4-fluoropyrrolidine-2-carboxylate hydrochloride (87 mg, 0.20 mmol) and (9,9-difluoro-9H-fluorene-3-carbonyl)glycine (60 mg, 0.20 mmol) in DMF (0.9 mL) was added DIPEA (130 mg, 1.0 mmol) and T₃P (153 mg, 0.24 mmol, 50% wt. in EtOAc) under N₂ atmosphere and the mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-10% MeOH in DCM) to give benzyl (2S,4R)-1-((9,9-difluoro-9H-fluorene-3-carbonyl)glycyl)-4-((((E)-6-ethoxy-6-oxohex-2-en-1-yl)oxy)methyl)-4-fluoropyrrolidine-2-carboxylate (100 mg, yield 73.7%) as a colorless oil. LC/MS (ESI) m/z: 679 (M+H)⁺.

### Step 4: (2S,4R)-1-((9,9-difluoro-9H-fluorene-3-carbonyl)glycyl)-4-(((6-ethoxy-6-oxohexyl)oxy) methyl)-4-fluoropyrrolidine-2-carboxylic acid (7)

To a solution of benzyl (2S,4R)-1-((9,9-difluoro-9H-fluorene-3-carbonyl)glycyl)-4-((((E)-6-ethoxy-6-oxohex-2-en-1-yl)oxy)methyl)-4-fluoropyrrolidine-2-carboxylate (60 mg, 0.088 mmol) in EtOAc (2 mL) was added PtO₂ (10 mg, 0.042 mmol) and the mixture was degassed under N₂ atmosphere for three times and stirred under a H₂ balloon at room temperature for 30 minutes. The mixture was filtered, and the filtrate was concentrated to dryness under reduced pressure to give (2S,4R)-1-((9,9-difluoro-9H-fluorene-3-carbonyl)glycyl)-4-(((6-ethoxy-6-oxohexyl)oxy) methyl)-4-fluoropyrrolidine-2-carboxylic acid (41 mg, yield 78.5%) as a colorless oil, which was used directly in the next step. LC/MS (ESI) (m/z): 591 (M+H)⁺.

### Step 5: ethyl 6-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)hexanoate (9)

To a mixture of (2S,4R)-1-((9,9-difluoro-9H-fluorene-3-carbonyl)glycyl)-4-(((6-ethoxy-6-oxohexyl)oxy)methyl)-4-fluoropyrrolidine-2-carboxylic acid (40 mg, 0.068 mmol) and (R)-5-(1-aminoethyl) thiophene-3-carboximidamide hydrochloride (17 mg, 0.081 mmol) in DMF (1 mL) was added DIPEA (27 mg, 0.21 mmol) and PyBOP (36 mg, 0.068 mmol) at 0° C. and the mixture was stirred under N₂ atmosphere at room temperature for 1 hour. The reaction mixture was diluted with water and extracted with EtOAc twice. The combined organic layers were washed with brine, dried over anhydrous Na₂SO₄, filtered, and concentrated to dryness under reduced pressure. The residue was purified by prep-TLC (DCM:MeOH=10:1) to give ethyl 6-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)hexanoate (40 mg, yield 79.6%) as a white solid. LCMS (ESI) (m/z): 742 (M+H)⁺.

### Step 6: 6-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)hexanoic acid (Compound 201)

To a solution of ethyl 6-(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methoxy)hexanoate (40 mg, 0.054 mmol) in MeOH (0.75 mL) and water (0.25 mL) was added LiOH·H₂O (5 mg, 0.12 mmol) at 0° C. and the mixture was stirred at room temperature for 3 hours. The mixture was acidified with 1 N aq·HCl to pH˜4 and concentrated to dryness under reduced pressure. The residue was purified by prep-HPLC to give Compound 201 (3.5 mg, yield 9.1%) as a white solid. ¹H NMR (400 MHz, CD₃OD) δ 8.49 (s, 1H), 8.21 (d, J=20.0 Hz, 2H), 7.88 (d, J=7.7 Hz, 1H), 7.74 (dd, J=17.7, 7.7 Hz, 2H), 7.67-7.63 (m, 1H), 7.57 (dd, J=17.1, 9.4 Hz, 2H), 7.45 (t, J=7.4 Hz, 1H), 5.28 (dd, J=7.3, 1.0 Hz, 1H), 4.65-4.60 (m, 1H), 4.32 (d, J=16.9 Hz, 1H), 4.19 (d, J=16.5 Hz, 1H), 4.08-3.95 (m, 2H), 3.76-3.68 (m, 2H), 3.55 (dd, J=9.8, 3.8 Hz, 2H), 2.26-2.22 (m, 2H), 1.61 (dd, J=6.4, 4.7 Hz, 6H), 1.42 (ddd, J=8.6, 3.5, 1.8 Hz, 2H). LCMS (ESI) (m/z): 714 (M+H)⁺.

### Scheme 193: Synthesis of (2S,4R)—N—((R)-1-(4-carbamimidoylthiophen-2-yl)ethyl)-1-((9,9-difluoro-9H-fluorene-3-carbonyl)glycyl)-4-fluoropyrrolidine-2-carboxamide (Compound 202)

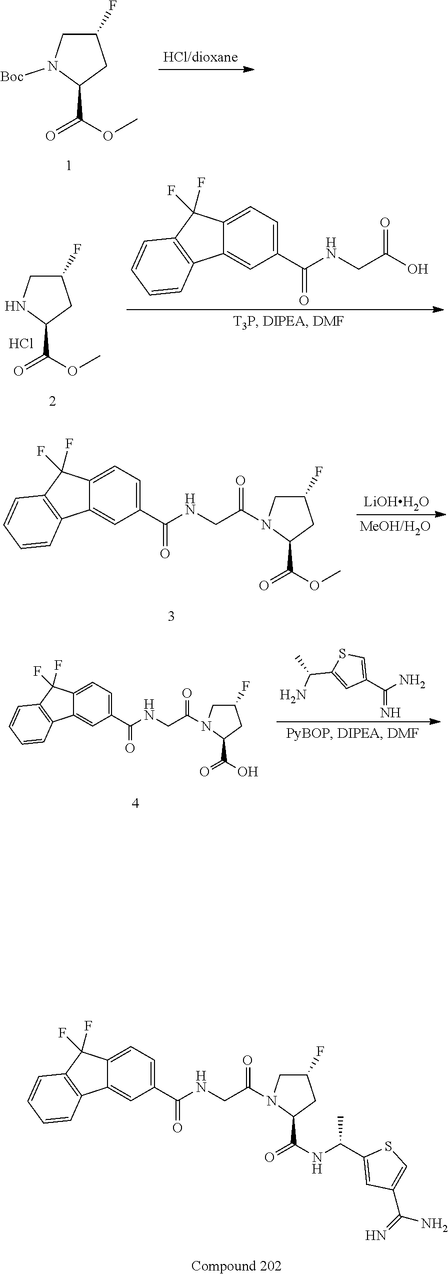

Compound 202 was prepared from 1-(tert-butyl) 2-methyl (2S,4R)-4-fluoropyrrolidine-1,2-dicarboxylate, (9,9-difluoro-9H-fluorene-3-carbonyl)glycine, and (R)-5-(1-aminoethyl)thiophene-3-carboximidamide based on the procedures set forth in Scheme 3. ¹H NMR (400 MHz, CD₃OD) δ 8.53 (s, 1H), 8.21 (dd, J=13.9, 12.5 Hz, 2H), 7.87 (t, J=9.6 Hz, 1H), 7.74 (dd, J=16.7, 7.8 Hz, 2H), 7.65 (d, J=7.4 Hz, 1H), 7.57 (dd, J=14.9, 7.6 Hz, 2H), 7.45 (t, J=7.6 Hz, 1H), 5.42 (d, J=52.5 Hz, 1H), 5.28 (d, J=7.0 Hz, 1H), 4.64-4.55 (m, 1H), 4.37 (d, J=16.8 Hz, 1H), 4.18 (d, J=16.8 Hz, 1H), 4.15-4.05 (m, 1H), 3.91 (dd, J=36.6, 12.5 Hz, 1H), 2.78-2.52 (m, 1H), 2.28-2.07 (m, 1H), 1.62 (dd, J=33.0, 7.0 Hz, 3H). LC/MS (ESI) m/z: 570 (M+H)⁺.

### Scheme 194: Synthesis of (S)—N—((R)-1-(4-carbamimidoylthiophen-2-yl)ethyl)-1-((9,9-difluoro-9H-fluorene-3-carbonyl)glycyl)-4-methylenepyrrolidine-2-carboxamide (Compound 203)

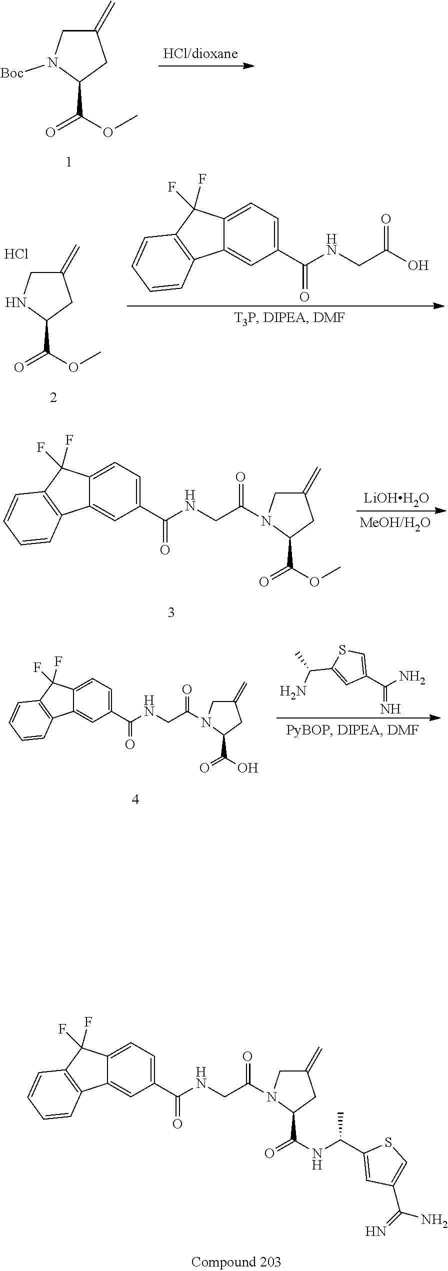

Compound 203 was prepared from 1-(tert-butyl) 2-methyl (S)-4-methylenepyrrolidine-1,2-dicarboxylate, (9,9-difluoro-9H-fluorene-3-carbonyl)glycine, and (R)-5-(1-aminoethyl)thiophene-3-carboximidamide based on the procedures set forth in Scheme 3. ¹H NMR (400 MHz, CD₃OD) δ 8.54 (s, 1H), 8.20 (d, J=7.5 Hz, 2H), 7.88 (t, J=7.8 Hz, 1H), 7.74 (dd, J=17.5, 7.9 Hz, 2H), 7.66 (d, J=7.0 Hz, 1H), 7.58 (t, J=7.5 Hz, 1H), 7.51-7.43 (m, 2H), 5.26 (d, J=7.1 Hz, 1H), 5.16-5.09 (m, 2H), 4.75-4.65 (m, 1H), 4.42 (t, J=10.9 Hz, 2H), 4.30-4.15 (m, 2H), 3.15-2.98 (m, 1H), 2.71 (t, J=21.6 Hz, 1H), 1.62 (dd, J=29.7, 6.9 Hz, 3H). LC/MS (ESI) m/z: 564 (M+H)⁺.

### Scheme 195: Synthesis of ((8S)—N-[(1R)-1-(4-carbamimidoylthiophen-2-yl)ethyl]-7-(2-{[9-(difluoromethoxy)-9H-fluoren-3-yl]formamido}acetyl)-1,4-dioxa-7-azaspiro[4.4]nonane-8-carboxamid (Compound 204)

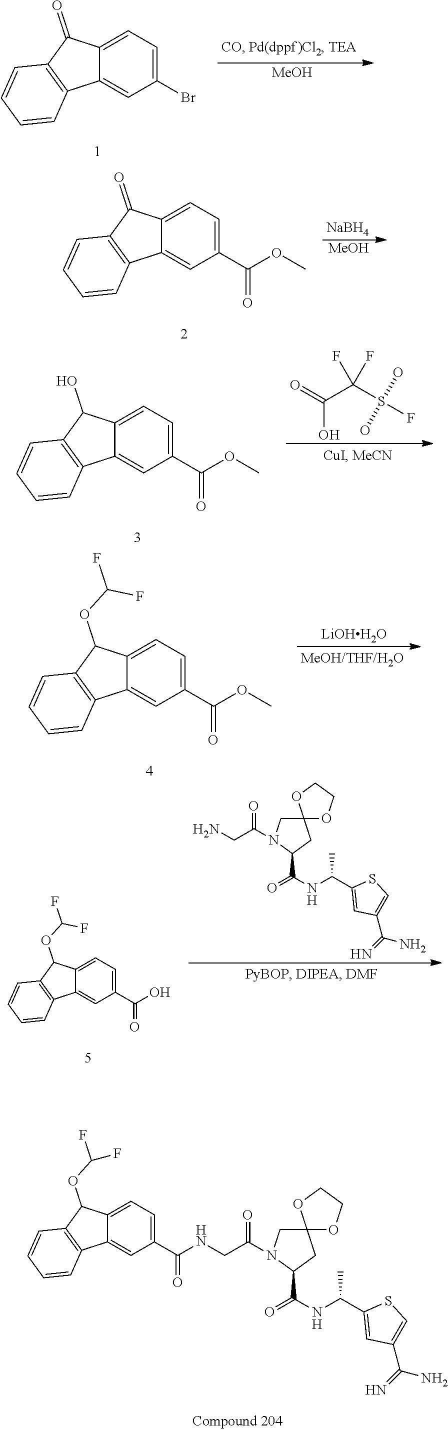

### Step 1: methyl 9-oxofluorene-3-carboxylate (2)

To a mixture of 3-bromofluoren-9-one (2.0 g, 1.54 mmol) and TEA (2.34 g, 23.16 mmol) in MeOH (40 mL) was added Pd(dppf)Cl₂ (1.13 g, 1.54 mmol) and the mixture was stirred under CO atmosphere at 75° C. for 48 hours. The mixture was diluted with water and extracted with DCM twice. The combined organic layers were dried over anhydrous Na₂SO₄, filtered, and concentrated to dryness under reduced pressure. The residue was purified by flash chromatography (silica gel, 0-10% EtOAc in PE) to give methyl 9-oxofluorene-3-carboxylate (1.3 g, yield 70.7%) as a yellow solid. LC/MS (ESI) m/z: 239 (M+H)⁺.

### Step 2: methyl 9-hydroxy-9H-fluorene-3-carboxylate (3)

To a solution of methyl 9-oxofluorene-3-carboxylate (1.0 g, 4.20 mmol) in MeOH (10 mL) was added NaBH₄ (397 mg, 10.49 mmol) at 0° C. and the mixture was stirred at 0° C. for 1 hour. The mixture was quenched with saturated aq·NH₄Cl solution and extracted with EtOAc twice. The combined organic layers were washed with brine, dried over anhydrous Na₂SO₄, filtered, and concentrated under reduced pressure to give methyl 9-hydroxy-9H-fluorene-3-carboxylate (860 mg, yield 85.3%) as a white solid. LC/MS (ESI) m/z: 241 (M+H)⁺.

### Step 3: methyl 9-(difluoromethoxy)-9H-fluorene-3-carboxylate (4)

To a mixture of methyl 9-hydroxy-9H-fluorene-3-carboxylate (300 mg, 1.25 mmol) and CuI (285 mg, 1.50 mmol) in MeCN (7 mL) was added difluoro(fluorosulfonyl)acetic acid (267 mg, 1.50 mmol) dropwisely at 50° C. and the mixture was stirred at 50° C. for 2 hours. The mixture was quenched with ice-water and extracted with EtOAc twice. The combined organic layers were washed with brine, dried with anhydrous Na₂SO₄, filtered, and concentrated under reduce pressure. The residue was purified by prep-TLC (PE:EtOAc=5:1) to give methyl 9-(difluoromethoxy)-9H-fluorene-3-carboxylate (110 mg, yield 30.4%) as a light oil. LC/MS (ESI) m/z: 291 (M+H)⁺.

### Step 4: 9-(difluoromethoxy)-9H-fluorene-3-carboxylic acid (5)

To a solution of methyl 9-(difluoromethoxy)-9H-fluorene-3-carboxylate (60 mg, 0.21 mmol) in MeOH (4 mL) and THF (1 mL) was added a solution of LiOH·H₂O (17 mg, 0.41 mmol) in H₂O (2 mL) at 0° C. and the mixture was stirred at room temperature for 1 hour. The mixture was acidified with 1 N aq·HCl to pH ˜3 and extracted with EtOAc twice. The combined organic layers were washed with brine, dried over anhydrous Na₂SO₄, filtered, and concentrated to dryness under reduced pressure to give 9-(difluoromethoxy)-9H-fluorene-3-carboxylic acid (20 mg, yield 35.0%) as a yellow oil, which was used directly in the next step. LC/MS (ESI) m/z: 275 (M−H)⁻.

### Step 5: ((8S)—N-[(1R)-1-(4-carbamimidoylthiophen-2-yl)ethyl]-7-(2-{[9-(difluoromethoxy)-9H-fluoren-3-yl]formamido)acetyl}-1,4-dioxa-7-azaspiro[4.4]nonane-8-carboxamid (Compound 204)

To a mixture of 9-(difluoromethoxy)-9H-fluorene-3-carboxylic acid (19 mg, 0.068 mmol) and (8S)-7-(2-aminoacetyl)-N-[(1R)-1-(4-carbamimidoylthiophen-2-yl)ethyl]-1,4-dioxa-7-azaspiro[4.4]nonane-8-carboxamide (20 mg, 0.052 mmol) in DMF (2 mL) was added DIPEA (27 mg, 0.021 mmol) and PyBOP (33 mg, 0.063 mmol) at room temperature and the mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and prep-HPLC to give Compound 204 (0.5 mg, yield 1.5%) as a white solid. ¹H NMR (400 MHz, CD₃OD) δ 8.22 (d, J=4.4 Hz, 2H), 7.81 (dd, J=23.2, 23.2 Hz, 2H), 7.65 (dd, J=22.4, 22.4 Hz, 2H), 7.53-7.46 (m, 2H), 7.40 (t, J=7.2 Hz, 1H), 6.78 (t, J=74.2 Hz, 1H), 6.20 (s, 1H), 5.32-5.25 (m, 1H), 4.59-4.56 (m, 1H), 4.26-4.17 (m, 2H), 4.03-3.96 (m, 4H), 3.85-3.77 (m, 2H), 2.49-2.39 (m, 1H), 2.21 (dd, J=13.2, 13.2 Hz, 1H), 1.58 (d, J=6.8 Hz, 3H). LC/MS (ESI) m/z: 640 (M+H)⁺.

### Scheme 196: Synthesis of (2S,4R)—N—((R)-1-(4-carbamimidoylthiophen-2-yl)ethyl)-1-((dibenzo[b,d]furan-2-carbonyl)glycyl)-4-(difluoromethoxy)pyrrolidine-2-carboxamide (Compound 205)

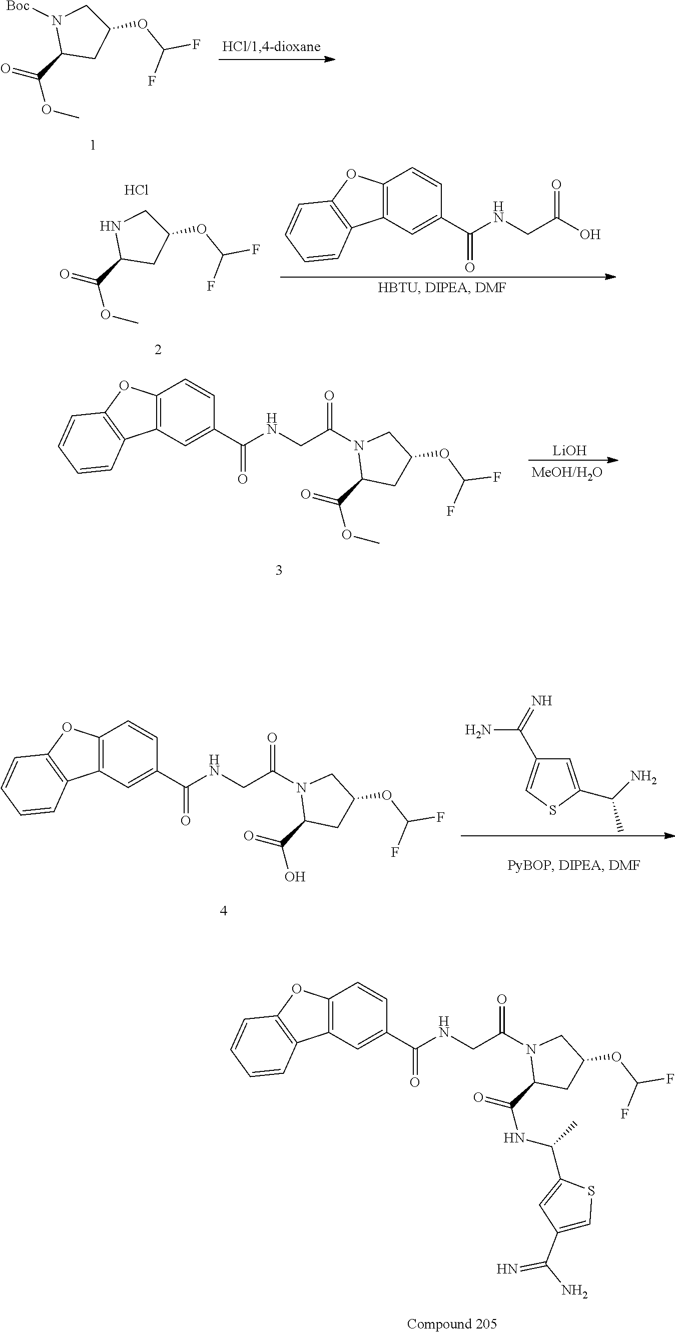

Compound 205 was prepared from 1-(tert-butyl) 2-methyl (2S,4R)-4-(difluoromethoxy)pyrrolidine-1,2-dicarboxylate, (dibenzo[b,d]furan-2-carbonyl)glycine, and (R)-5-(1-aminoethyl)thiophene-3-carboximidamide based on the procedures set forth in Scheme 3. ¹H NMR (400 MHz, CD₃OD) δ 8.61 (d, J=6.3 Hz, 2H), 8.54 (d, J=5.6 Hz, 1H), 8.21 (d, J=17.6 Hz, 1H), 8.10 (dd, J=15.1, 5.7 Hz, 3H), 7.86 (d, J=8.9 Hz, 1H), 7.53 (d, J=4.1 Hz, 3H), 6.74-6.33 (m, 1H), 5.32-5.22 (m, 1H), 5.05 (s, 1H), 4.61 (s, 1H), 4.36 (d, J=16.7 Hz, 1H), 4.24 (d, J=16.7 Hz, 1H), 3.98 (dd, J=16.0, 11.7 Hz, 2H), 2.50 (s, 1H), 2.29-2.23 (m, 1H), 1.59 (d, J=7.0 Hz, 3H). LCMS (ESI) (m/z): 584 (M+H)⁺.

### Scheme 197: Synthesis of (2S,4R)-1-((anthracene-2-carbonyl)glycyl)-N—((R)-1-(4-carbamimidoylthiophen-2-yl)ethyl)-4-(difluoromethoxy)pyrrolidine-2-carboxamide (Compound 206)

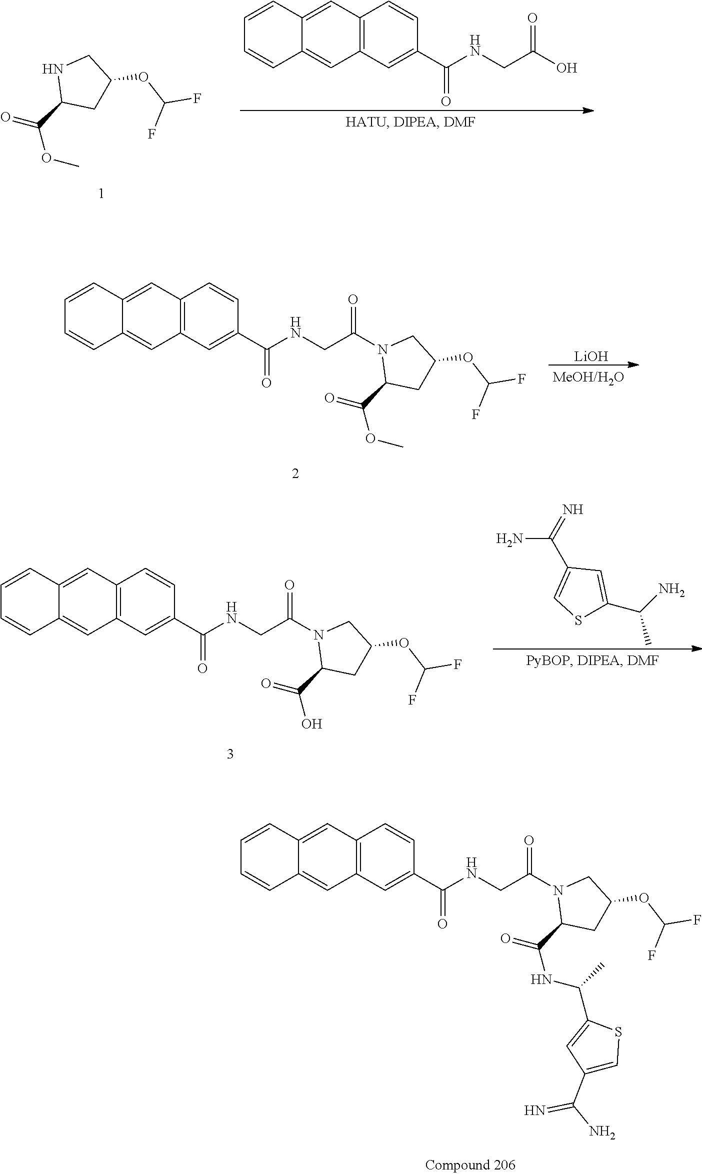

Compound 206 was prepared from methyl (2S,4R)-4-(difluoromethoxy)pyrrolidine-2-carboxylate, (anthracene-2-carbonyl)glycine, and (R)-5-(1-aminoethyl)thiophene-3-carboximidamide based on the procedures set forth in Scheme 3. ¹H NMR (400 MHz, CD₃OD) δ 8.61 (d, J=6.3 Hz, 2H), 8.54 (d, J=5.6 Hz, 1H), 8.21 (d, J=17.6 Hz, 1H), 8.10 (dd, J=15.1, 5.7 Hz, 3H), 7.86 (d, J=8.9 Hz, 1H), 7.53 (d, J=4.1 Hz, 3H), 6.74-6.33 (m, 1H), 5.32-5.22 (m, 1H), 5.05 (s, 1H), 4.61 (s, 1H), 4.36 (d, J=16.7 Hz, 1H), 4.24 (d, J=16.7 Hz, 1H), 3.98 (dd, J=16.0, 11.7 Hz, 2H), 2.50 (s, 1H), 2.29-2.23 (m, 1H), 1.59 (d, J=7.0 Hz, 3H). LCMS (ESI) (m/z): 594 (M+H)⁺.

### Scheme 198: (S)—N-((4-carbamimidoylthiophen-2-yl)methyl)-7-((3′,4′-difluoro-[1,1′-biphenyl]-4-carbonyl)glycyl)-1,4-dioxa-7-azaspiro[4.4]nonane-8-carboxamide (Compound 207)

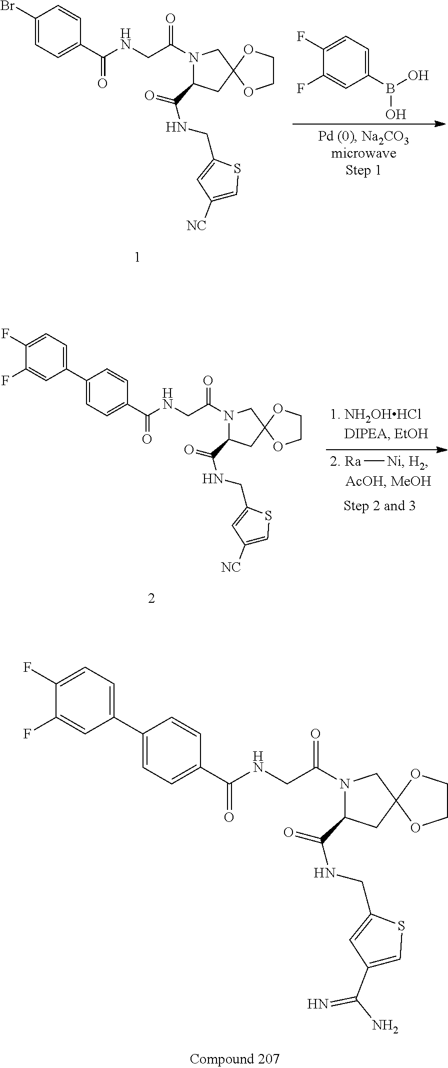

Compound 207 was prepared from (3,4-difluorophenyl)boronic acid, (8S)-7-[2-[(4-bromobenzoyl)amino]acetyl]-N-[(5-cyano-3-thienyl)methyl]-1,4-dioxa-7-azaspiro[4.4]nonane-8-carboxamide, and NH₂OH·HCl based on the procedures set forth in Scheme 103. LC/MS (ESI) m/z: 584 (M+H)+. ¹H NMR (400 MHz, DMSO-d₆) δ 8.92 (s, 1H), 8.64 (s, 1H), 8.51-8.43 (m, 2H), 8.24 (s, 1H), (m, 2H), 8.15 (s, 1H), 7.87 (d, J=7.8 Hz, 2H), 7.37-7.20 (m, 2H), 7.05-7.07 (t, J=9.1 Hz, 1H), 4.54-4.19 (m, 2H), 4.11 (d, J=14.2 Hz, 2H), 4.02-3.66 (m, 2H), 3.69-3.41 (m, 2H), 3.10-2.99 (m, 2H), 2.08-2.06 (m, 1H), 1.98 (dd, J=12.5, 6.7 Hz, 1H).

### Scheme 199: (S)—N-((4-carbamimidoylthiophen-2-yl)methyl)-7-((3′,5′-dichloro-[1,1′-biphenyl]-4-carbonyl)glycyl)-1,4-dioxa-7-azaspiro[4.4]nonane-8-carboxamide (Compound 208)

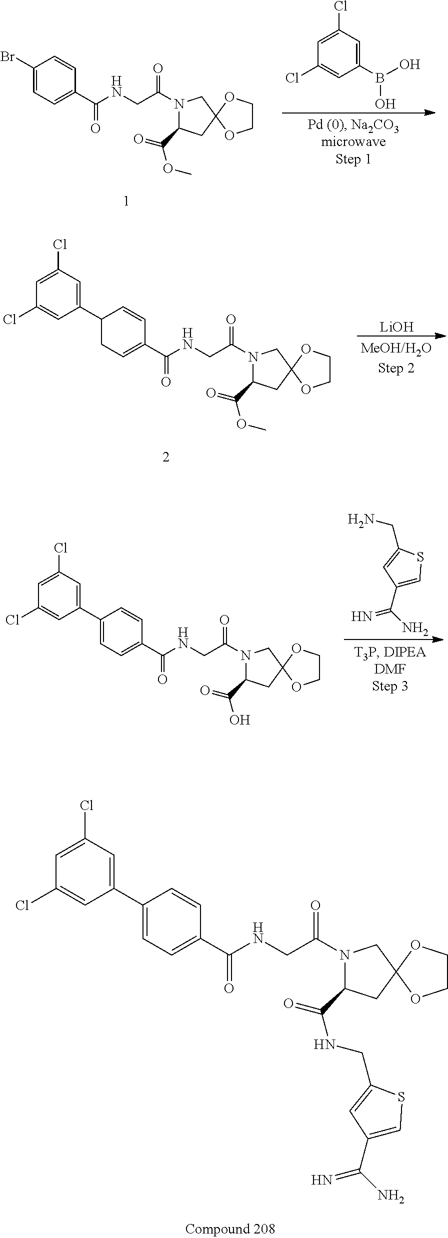

### Step 1: methyl (S)-7-((3,5′-dichloro-[1,1′-biphenyl]-4-carbonyl)glycyl)-1,4-dioxa-7-azaspiro[4.4]nonane-6-carboxylate (2)

A mixture of (3,5-dichlorophenyl)boronic acid (21.44 mg, 0.1124 mmol, 1.2 equiv.), methyl (8S)-7-[2-[(4-bromobenzoyl)amino]acetyl]-1,4-dioxa-7-azaspiro[4.4]nonane-8-carboxylate (40.00 mg, 0.09363 mmol, 1 equiv.), Pd(dppf)Cl₂·CH₂Cl₂ (7.80 mg, 0.00936 mmol, 0.1 equiv.), and Na₂CO₃ (19.85 mg, 0.1873 mmol, 2 equiv.) in 1,4-dioxane (3 mL) and water (0.3 mL) was stirred in a microwave reactor at 110° C. for 30 min. The reaction was cooled to room temperature and then filtered through Celite pad. The filtrate was diluted with water and EtOAc. The two layers were separated, and the aqueous layer was extracted with EtOAc (5 mL×2). Purification by combi-Flash; 4 g column, solvent A=CH₂Cl₂, solvent B=MeOH. 100% A to 5% B gave methyl (8S)-7-[2-[[4-(3,5-dichlorophenyl)benzoyl]amino]acetyl]-1,4-dioxa-7-azaspiro[4.4]nonane-8-carboxylate (32.00 mg, 0.06487 mmol, 69.28% yield). LC/MS (ESI) m/z: 494 (M+H)+.

### Step 2: (S)-7-((3,5′-dichloro-[1,1′-biphenyl]-4-carbonyl)glycyl)-1,4-dioxa-7-azaspiro[4.4]nonane-8-carboxylic acid (3)

To a solution of methyl (8S)-7-[2-[[4-(3,5-dichlorophenyl)benzoyl]amino]acetyl]-1,4-dioxa-7-azaspiro[4.4]nonane-8-carboxylate (32.00 mg, 0.06487 mmol, 1.00 equiv.) in MeOH (1.5 mL) water (0.5 mL) was added LiOH (1.902 mg, 0.07783 mmol, 1.2 equiv.). The reaction was stirred at room temperature overnight. The reaction was then concentrated to remove MeOH and diluted with EtOAc. The pH was adjusted to 1 using 1 N HCl. The two layers were separated, and the aqueous layer was extracted with EtOAc (5 mL×2). The combined organic extracts were washed with brine, dried over Na₂SO₄, and concentrated to give (8S)-7-[2-[[4-(3,5-dichlorophenyl)benzoyl]amino]acetyl]-1,4-dioxa-7-azaspiro[4.4]nonane-8-carboxylic acid (25.00 mg, 0.05216 mmol, 80.41% yield) as a brown solid which was used in the next step without further purification. LC/MS (ESI) m/z: 480 (M+H)⁺.

### Step 3: (S)—N-((4-carbamimidoylthiophen-2-yl)methyl)-7-((3′,5′-dichloro-[1,1′-biphenyl]-4-carbonyl)glycyl)-1,4-dioxa-7-azaspiro[4.4]nonane-8-carboxamide (Compound 208)

To a solution of (8S)-7-[2-[[4-(3,5-dichlorophenyl)benzoyl]amino]acetyl]-1,4-dioxa-7-azaspiro[4.4]nonane-8-carboxylic acid (25.00 mg, 0.05216 mmol 1.0 equiv.) in DMF (1 mL) was added 5-(aminomethyl)thiophene-3-carboximidamide. HCl (12.00 mg, 0.06260 mmol, 1.2 equiv.), T3P, (50 mass %) in EtOAc (46.8 μL, 0.0782 mmol, 1.5 equiv.), and DIPEA (18.2 μL, 0.104 mmol, 3.0 equiv.). The reaction mixture was stirred at room temperature for 30 minutes and then purified directly by HPLC to give Compound 208 (3.00 mg, 0.004866 mmol, 9.329% yield). LC/MS (ESI) m/z: 616 (M)⁺ and 618. ¹H NMR (400 MHz, DMSO) δ 9.01 (s, 1H), 8.81 (s, 1H), 8.64 (s, 2H), 8.53 (s, 1H), 8.32 (d, J=12.9 Hz, 1H), 8.00 (d, J=8.0 Hz, 2H), 7.91 (d, J=8.0 Hz, 2H), 7.68 (s, 1H), 7.49 (s, 1H). 4.71 (d, J=8.2 Hz, 1H), 4.51-4.34 (m, 2H), 4.19 (dd, J=16.7, 5.4 Hz, 2H), 4.02 (d, J=5.2 Hz, 1H), 4.00-3.83 (m, 2H), 3.80 (d, J=10.8 Hz, 2H), 3.68-3.56 (m, 1H), 2.32 (dd, J=13.5, 8.6 Hz, 1H), 2.11-1.95 (m, 1H).

### Scheme 200: (S)—N-((4-carbamimidoylthiophen-2-yl)methyl)-7-((4-(6-fluoropyridin-3-yl)benzoyl)glycyl)-1,4-dioxa-7-azaspiro[4.4]nonane-8-carboxamide (Compound 209)

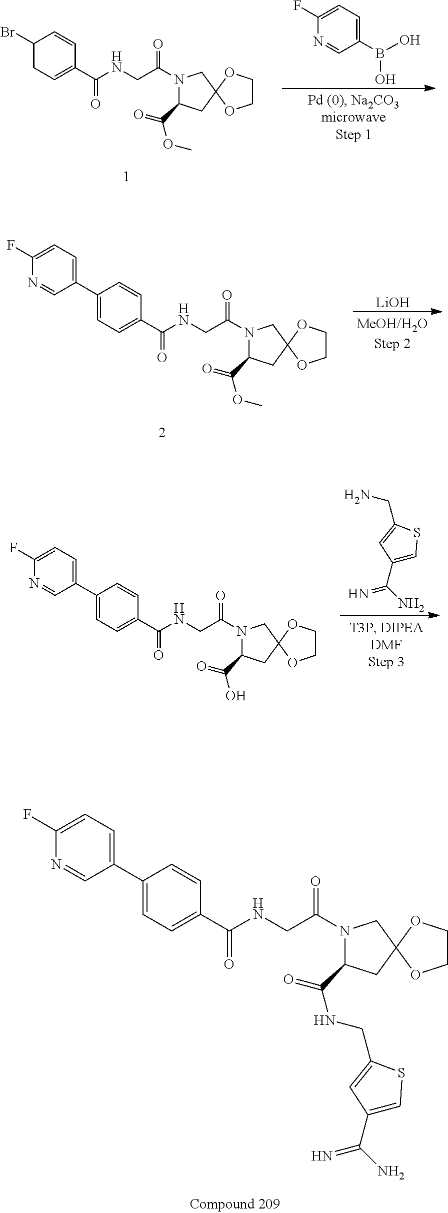

Compound 209 was prepared from (6-fluoro-3-pyridyl)boronic, methyl (8S)-7-[2-[(4-bromobenzoyl)amino]acetyl]-1,4-dioxa-7-azaspiro[4.4]nonane-8-carboxylate, and 5-(aminomethyl)thiophene-3-carboximidamide. HCl based on the procedures set forth in Scheme 199. LC/MS (ESI) m/z: 567 (M+H)⁺. ¹H NMR (400 MHz, DMSO) δ 8.91 (s, 1H), 8.70 (s, 1H), 8.55 (d, J=14.2 Hz, 2H), 8.38-8.22 (m, 2H), 7.91 (d, J=7.5 Hz, 2H), 7.78 (d, J=8.1 Hz, 2H), 7.48-7.37 (m, 1H), 7.25 (d, J=8.5 Hz, 1H), 4.71 (d, J=8.2 Hz, 1H), 4.51-4.34 (m, 2H), 4.19 (dd, J=16.7, 5.4 Hz, 2H), 4.02 (d, J=5.2 Hz, 1H), 4.00-3.83 (m, 2H), 3.80 (d, J=10.8 Hz, 2H), 3.68-3.56 (m, 1H), 2.25-2.22 (m, 1H), 2.01-1.98 (m, 1H).

### Scheme 201: (S)—N-((4-carbamimidoylthiophen-2-yl)methyl)-7-((3′-fluoro-4′-methyl-[1,1′-biphenyl]-4-carbonyl)glycyl)-1,4-dioxa-7-azaspiro[4.4]nonane-8-carboxamide (Compound 210)

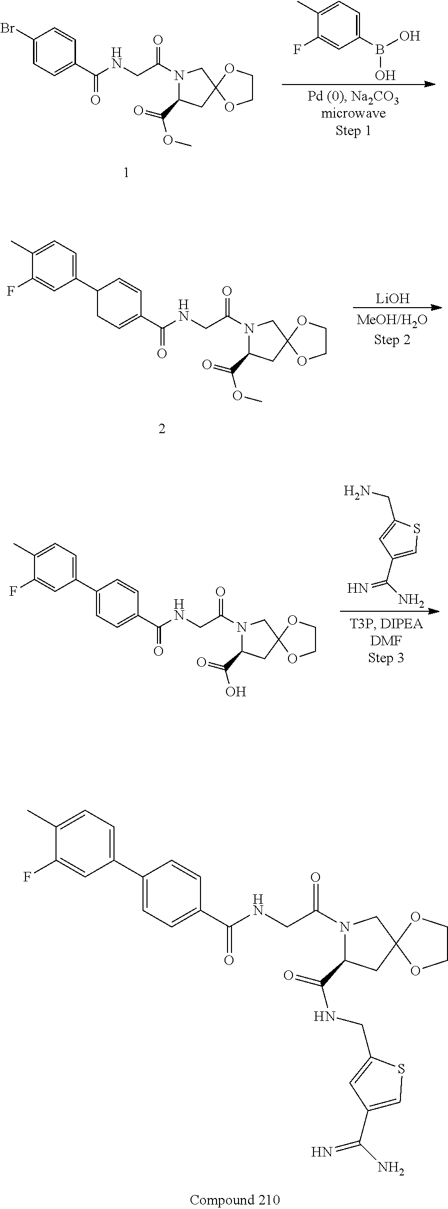

Compound 210 was prepared from (3-fluoro-4-methyl-phenyl)boronic acid, methyl (8S)-7-[2-[(4-bromobenzoyl)amino]acetyl]-1,4-dioxa-7-azaspiro[4.4]nonane-8-carboxylate, and 5-(aminomethyl)thiophene-3-carboximidamide. HCl based on the procedures set forth in Scheme 199. LC/MS (ESI) m/z: 580 (M+H)⁺. ¹H NMR (400 MHz, DMSO) δ 8.66 (s, 1H), 8.54 (s, 1H), 8.41 (s, 1H), 8.23 (s, 1H), 7.88 (d, J=7.9 Hz, 2H), 7.73 (d, J=7.8 Hz, 2H), 7.46 (dd, J=16.9, 9.7 Hz, 2H), 7.33 (t, J=8.0 Hz, 1H), 4.33 (d, J=16.9 Hz, 2H), 4.11 (d, J=16.4 Hz, 2H), 3.96-3.78 (m, 3H), 3.73 (d, J=10.8 Hz, 2H), 3.69-3.53 (m, 3H), 2.21 (s, 3H), 1.98 (dd, J=12.4, 6.7 Hz, 1H), 1.78-1.42 (m, 1H).

### Scheme 202: (S)—N-((4-carbamimidoylthiophen-2-yl)methyl)-7-((4-(6-fluoro-2-methylpyridin-3-yl)benzoyl)glycyl)-1,4-dioxa-7-azaspiro[4.4]nonane-8-carboxamide (Compound 211)

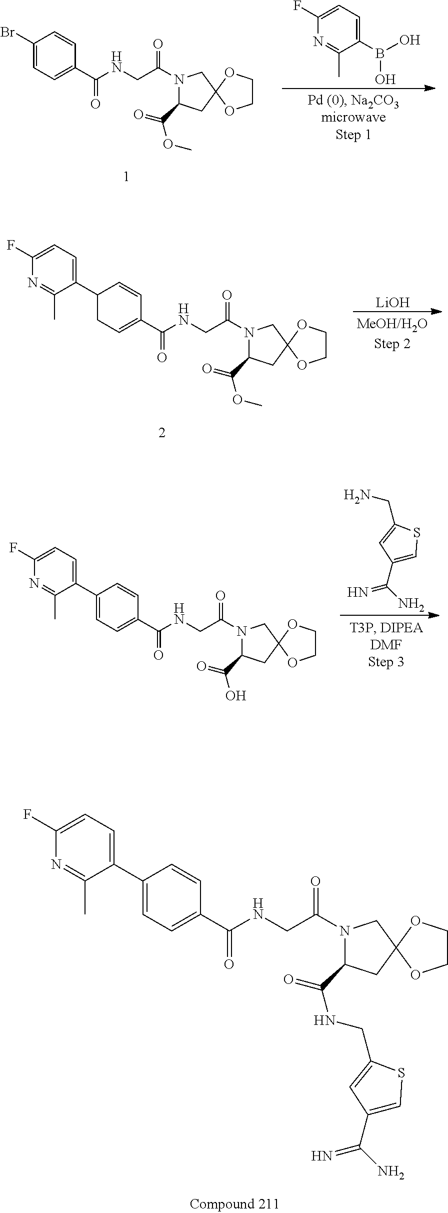

Compound 211 was prepared from (6-fluoro-2-methyl-3-pyridyl)boronic acid, methyl (8S)-7-[2-[(4-bromobenzoyl)amino]acetyl]-1,4-dioxa-7-azaspiro[4.4]nonane-8-carboxylate, and 5-(aminomethyl)thiophene-3-carboximidamide. HCl based on the procedures set forth in Scheme 199. LC/MS (ESI) m/z: 581 (M+H)⁺. H NMR (400 MHz, DMSO) δ 8.75 (t, J=5.4 Hz, 1H), 8.60 (t, J=5.9 Hz, 1H), 8.42 (s, 1H), 8.32 (d, J=13.8 Hz, 1H), 7.94 (d, J=7.9 Hz, 2H), 7.84 (t, J=8.2 Hz, 1H), 7.58-7.38 (m, 3H), 7.08 (dd, J=8.4, 3.1 Hz, 1H), 4.52-4.29 (m, 3H), 4.24-4.06 (m, 3H), 3.84-3.64 (m, 3H), 2.97 (p, J=6.6 Hz, 2H), 2.37 (s, 3H), 2.25-2.22 (m, 1H), 2.04 (dd, J=13.1, 6.9 Hz, 1H).

### Scheme 203: (S)-4′-((2-(8-(((4-carbamimidoylthiophen-2-yl)methyl)carbamoyl)-1,4-dioxa-7-azaspiro[4.4]nonan-7-yl)-2-oxoethyl)carbamoyl)-4-fluoro-[1,1′-biphenyl]-3-carboxylic acid (Compound 212)

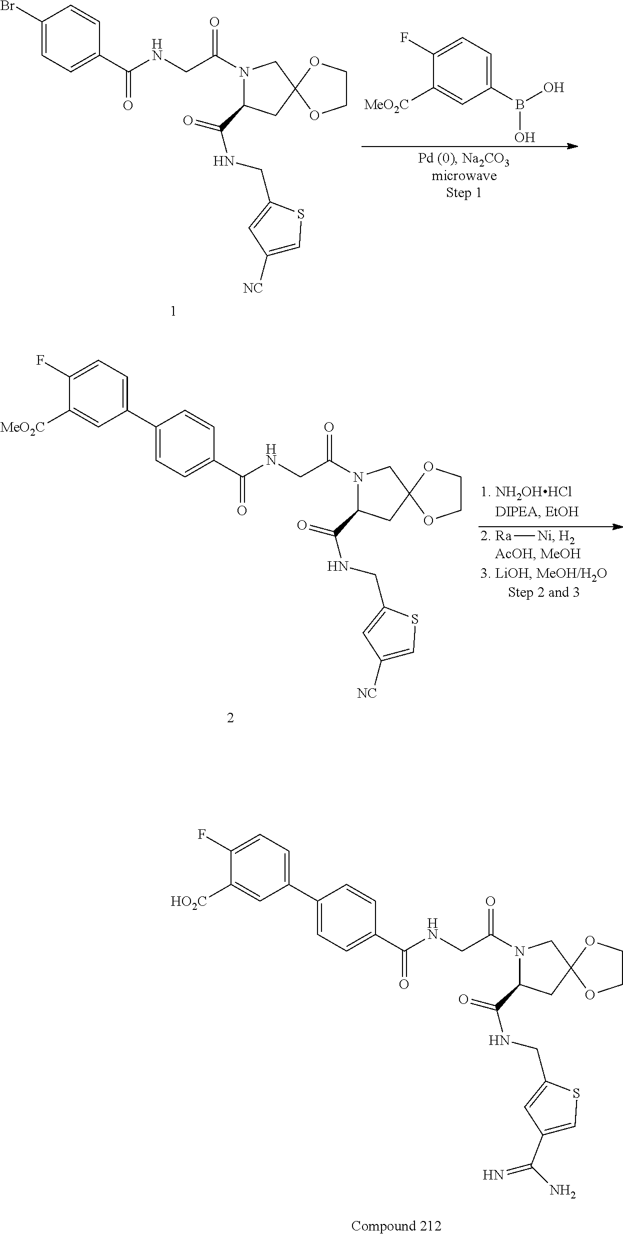

Compound 212 was prepared from (4-fluoro-3-methoxycarbonyl-phenyl)boronic acid, methyl (8S)-7-[2-[(4-bromobenzoyl)amino]acetyl]-1,4-dioxa-7-azaspiro[4.4]nonane-8-carboxylate, and NH₂OH·HCl based on the procedures set forth in Scheme 103. LC/MS (ESI) m/z: 610 (M+H)⁺. ¹H NMR (400 MHz, DMSO) δ 11.53 (s, 1H), 9.25 (s, 1H), 9.00 (s, 1H), 8.81 (s, 1H), 8.70 (s, 1H), 8.60-8.57 (m, 1H), 8.16-8.05 (m, 2H), 8.00-7.67 (m, 3H), 7.38 (s, 1H), 4.69-4.51 (m, 2H), 4.36-3.83 (m, 3H), 2.87-2.60 (m, 3H), 2.58-2.47 (m, 3H), 2.33-2.24 (m, 1H), 1.71-1.24 (m, 1H).

### Scheme 204: (S)-4′-((2-(8-(((4-carbamimidoylthiophen-2-yl)methyl)carbamoyl)-1,4-dioxa-7-azaspiro[4.4]nonan-7-yl)-2-oxoethyl)carbamoyl)-3-fluoro-[1,1′-biphenyl]-4-carboxylic acid (Compound 213)

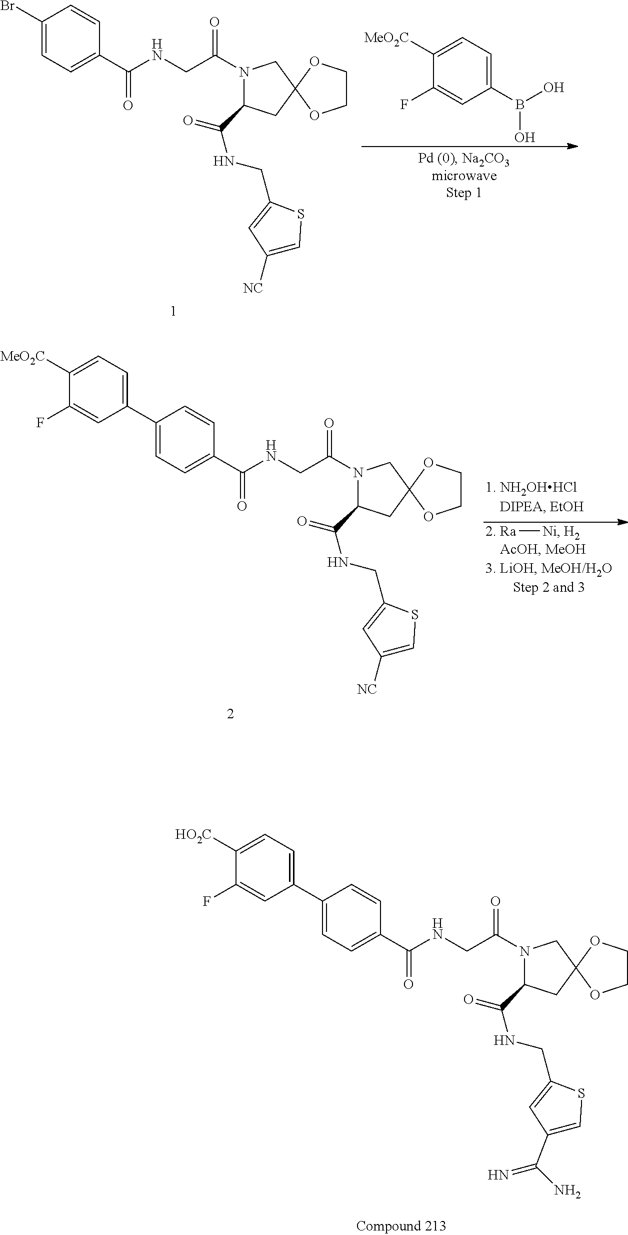

Compound 213 was prepared from (3-fluoro-4-methoxycarbonyl-phenyl)boronic acid, methyl (8S)-7-[2-[(4-bromobenzoyl)amino]acetyl]-1,4-dioxa-7-azaspiro[4.4]nonane-8-carboxylate, and NH₂OH·HCl based on the procedures set forth in Scheme 103. LC/MS (ESI) m/z: 610 (M+H)⁺. ¹H NMR (400 MHz, DMSO) δ 11.97 (s, 1H), 8.89 (s, 1H), 8.88-8.67 (m, 1H), 8.47 (s, 1H), 8.40-8.23 (m, 1H), 7.94-7.80 (m, 1H), 7.75 (t, J=10.7 Hz, 1H), 7.69-7.56 (m, 1H), 7.50 (s, 1H), 7.44-7.17 (m, 1H), 4.68-4.20 (m, 2H), 3.89-3.80 (m, 3H), 3.76-3.56 (m, 3H), 2.87-2.65 (m, 3H), 2.34-1.91 (m, 1H), 1.62-1.52 (m, 1H).

### Scheme 205. (S)-4′-((2-(8-(((4-carbamimidoylthiophen-2-yl)methyl)carbamoyl)-1,4-dioxa-7-azaspiro[4.4]nonan-7-yl)-2-oxoethyl)carbamoyl)-[1,1′-biphenyl]-4-carboxylic acid (Compound 214)

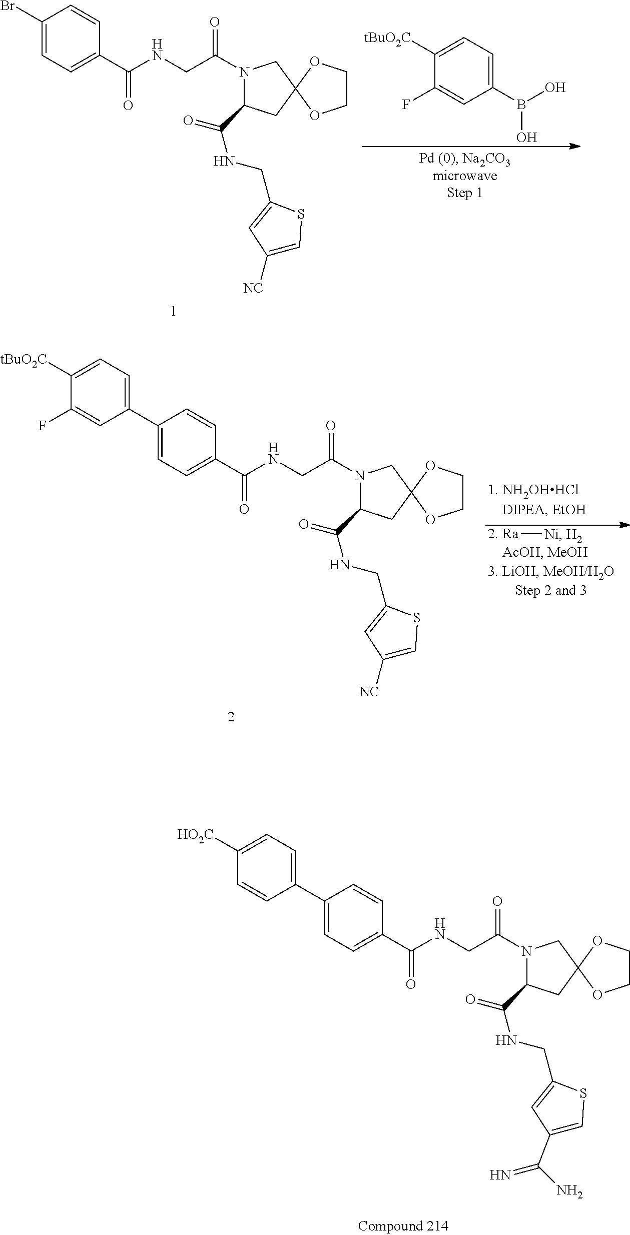

Compound 214 was prepared from (4-tert-butoxycarbonylphenyl)boronic acid, (8S)-7-[2-[(4-bromnobenzoyl)amino]acetyl]-N-[(4-cyano-2-thienyl)methyl]-1,4-dioxa-7-azaspiro[4.4]nonane-8-carboxamide, and NH₂OH·HCl based on the procedures set forth in Scheme 103. LC/MS (ESI) m/z: 602 (M+H)⁺. ¹H NMR (400 MHz, D₂O) δ 8.31 (s, 1H), 7.98-7.80 (m, 1H), 7.76 (d, J=7.6 Hz, 1H), 7.71 (d, J=7.7 Hz, 1H), 7.69-7.64 (m, 1H), 7.61 (d, J=8.4 Hz, 1H), 7.09 (s, 1H), 4.50-4.26 (m, 2H), 4.07 (q, J=13.8 Hz, 1H), 3.91-3.75 (m, 2H), 3.69 (d, J=10.7 Hz, 2H), 3.20 (d, J=1.2 Hz, 3H), 2.58-2.32 (m, 2H), 2.20-2.03 (m, 1H), 1.49-1.21 (m, 1H).

### Scheme 206. (S)—N-((4-carbamimidoylthiophen-2-yl)methyl)-7-((2′-fluoro-4′-(methylsulfonyl)-[1,1′-biphenyl]-4-carbonyl)glycyl)-1,4-dioxa-7-azaspiro[4.4]nonane-8-carboxamide (Compound 215)

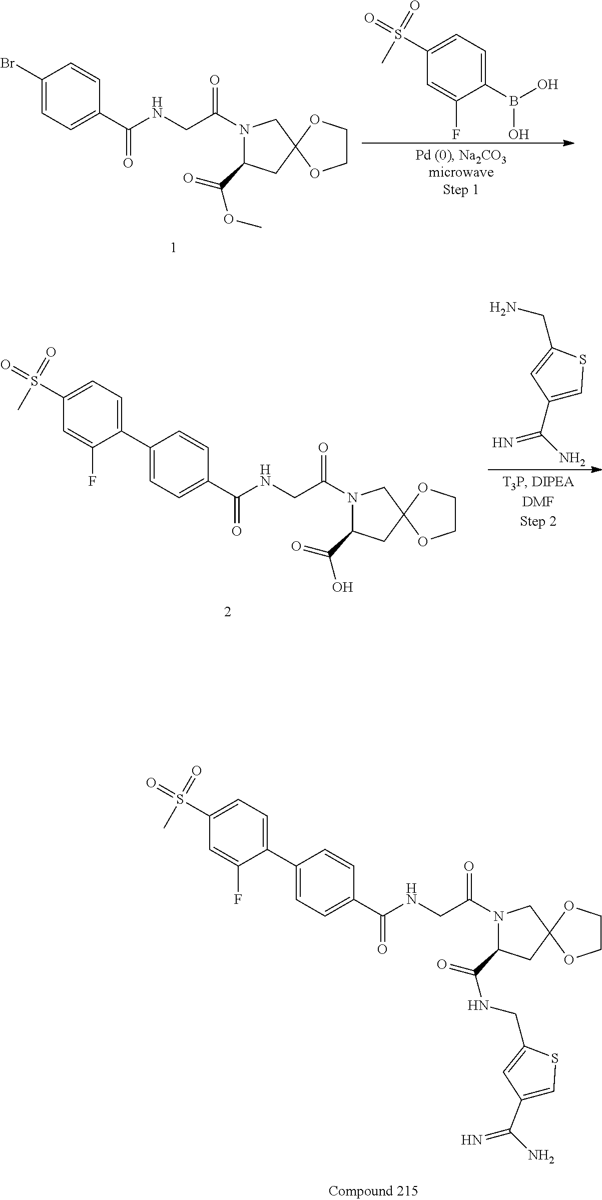 [SMILES: COC(=O)[C@@H]1CC2(CN1C(=O)CNC(=O)c1ccc(Br)cc1)OCCO2 ; CS(=O)(=O)c1ccc(B(O)O)c(F)c1 ; CS(=O)(=O)c1ccc(-c2ccc(C(=O)NCC(=O)N3CC4(C[C@H]3C(=O)O)OCCO4)cc2)c(F)c1 ; N=C(N)c1csc(CN)c1 ; CS(=O)(=O)c1ccc(-c2ccc(C(=O)NCC(=O)N3CC4(C[C@H]3C(=O)NCc3cc(C(=N)N)cs3)OCCO4)cc2)c(F)c1]

### Step 1: (S)-7-((2′-fluoro-4′-(methylsulfonyl)-[1,1′-biphenyl]-4-carbonyl)glycyl)-1,4-dioxa-7-azaspiro[4.4]nonane-6-carboxylic acid (2)

A mixture of (2-fluoro-4-methylsulfonyl-phenyl)boronic acid (27.56 mg, 0.1264 mmol, 1.2 equiv.), methyl (8S)-7-[2-[(4-bromobenzoyl)amino]acetyl]-1,4-dioxa-7-azaspiro[4.4]nonane-8-carboxylate (45 mg, 0.1053 mmol, 0.1 equiv.), Pd(dppf)Cl₂·CH₂Cl₂ (8.78 mg, 0.0105 mmol, 0.1 equiv.), Na₂CO₃ (44.66 mg, 0.4214 mmol, 4 equiv.) in 1,4-dioxane (3 mL) and water (0.3 mL) was stirred in a microwave reactor at 110° C. for 30 min. The reaction was cooled to room temperature and then filtered through celite pad. The filtrate was diluted with water, extracted with EtOAc (5 mL×2) to remove traces of methyl ester product. The pH of the aqueous layer was adjusted to 1 using 1 N HCl and then extracted with EtOAc (5 mL×3). The combined organic extracts were washed with brine, dried over Na₂SO₄, and concentrated to dryness to give (8S)-7-[2-[[4-(2-fluoro-4-methylsulfonyl-phenyl)benzoyl]amino]acetyl]-1,4-dioxa-7-azaspiro[4.4]nonane-8-carboxylic acid (30.00 mg, 0.05923 mmol,56.23% yieldyield) as a brown solid which was used in the next step without further purification. LC/MS (ESI) m/z: 507 (M+H)⁺.

### Step 2. (S)—N-((4-carbamimidoylthiophen-2-yl)methyl)-7-((2′-fluoro-4′-(methylsulfonyl)-[1,1′-biphenyl]-4-carbonyl)glycyl)-1,4-dioxa-7-azaspiro[4.4]nonane-8-carboxamide (Compound 215)

To a solution of (8S)-7-[2-[[4-(2-fluoro-4-methylsulfonyl-phenyl)benzoyl]amino]acetyl]-1,4-dioxa-7-azaspiro[4.4]nonane-8-carboxylic acid (25.00 mg, 0.04936 mmol, 1.0 equiv.) in DMF (1 mL) was added 5-(aminomethyl)thiophene-3-carboximidamide. HCl (11.35 mg, 0.05921 mmol, 1.2 equiv.), T3P, (50 mass %) in EtOAc (44.30 μL, 0.0740 mmol, 1.5 equiv.), and DIPEA (25.80 μL, 0.148 mmol, 3.0 equiv.). The reaction mixture was stirred at room temperature for 30 minutes and then purified directly by HPLC to give Compound 215 (2.8 mg, 0.0043 mmol, 8.8% yield) LC/MS (ESI) m/z: 644 (M+H)⁺. ¹H NMR (400 MHz, DMSO) δ 8.94 (s, 1H), 8.75 (s, 1H), 8.55 (s, 1H), 8.39 (s, 2H), 8.27 (s, 2H), 7.95 (s, 1H), 7.93-7.75 (m, 3H), 7.56 (s, 1H), 7.47 (s, 1H), 7.40 (d, J=12.0 Hz, 1H), 4.66-4.36 (m, 2H), 4.32 (d, J=7.4 Hz, 2H), 4.13 (d, J=15.0 Hz, 2H), 3.92-3.80 (m, 2H), 3.74 (d, J=10.7 Hz, 1H), 3.67-3.51 (m, 2H), 3.25-3.17 (m, 2H), 3.16 (s, 3H), 2.25 (t, J=11.1 Hz, 1H), 2.04-1.96 (m, 1H).

### Scheme 207. (S)—N-((4-carbamimidoylthiophen-2-yl)methyl)-7-((4′-(isopropylsulfonyl)-[1,1′-biphenyl]-4-carbonyl)glycyl)-1,4-dioxa-7-azaspiro[4.4]nonane-8-carboxamide (Compound 216)

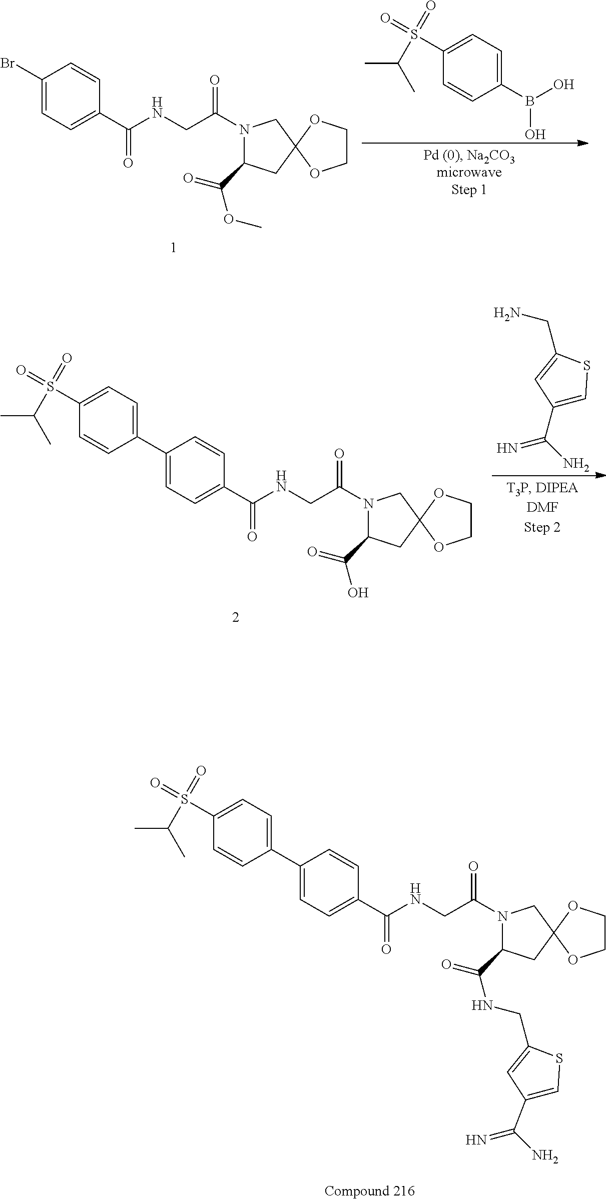 [SMILES: COC(=O)[C@@H]1CC2(CN1C(=O)CNC(=O)c1ccc(Br)cc1)OCCO2 ; CC(C)S(=O)(=O)c1ccc(B(O)O)cc1 ; CC(C)S(=O)(=O)c1ccc(-c2ccc(C(=O)NCC(=O)N3CC4(C[C@H]3C(=O)O)OCCO4)cc2)cc1 ; N=C(N)c1csc(CN)c1 ; CC(C)S(=O)(=O)c1ccc(-c2ccc(C(=O)NCC(=O)N3CC4(C[C@H]3C(=O)NCc3cc(C(=N)N)cs3)OCCO4)cc2)cc1]

Compound 216 was prepared from (4-isopropylsulfonylphenyl)boronic acid, methyl (8S)-7-[2-[(4-bromobenzoyl)amino]acetyl]-1,4-dioxa-7-azaspiro[4.4]nonane-8-carboxylate, and 5-(aminomethyl)thiophene-3-carboximidamide. HCl based on the procedures set forth in Scheme 206. LC/MS (ESI) m/z:654 (M+H)⁺. ¹H NMR (400 MHz, DMSO) δ 8.99 (s, 1H), 8.87-8.68 (m, 1H), 8.61 (s, 1H), 8.47-8.26 (m, 1H), 8.16-8.06 (m, 1H), 8.02 (dd, J=12.6, 8.1 Hz, 2H), 7.95 (d, J=8.2 Hz, 1H), 7.89 (d, J=8.0 Hz, 1H), 7.79 (d, J=7.9 Hz, 1H), 7.69 (d, J=9.0 Hz, 1H), 7.48 (s, 1H), 4.53-4.22 (m, 2H), 4.19 (dd, J=16.6, 5.6 Hz, 2H), 4.07-3.84 (m, 3H), 3.81 (d, J=10.8 Hz, 2H), 3.63 (d, J=10.8 Hz, 1H), 3.51-3.02 (m, 1H), 2.65-2.41 (m, 1H), 2.32 (dd, J=13.2, 8.8 Hz, 1H), 2.13-1.98 (m, 1H), 1.20 (d, J=6.8 Hz, 3H), 1.15 (d, J=6.7 Hz, 3H).

### Scheme 208. (S)—N-((4-carbamimidoylthiophen-2-yl)methyl)-7-((4′-methoxy-[1,1′-biphenyl]-4-carbonyl)glycyl)-1,4-dioxa-7-azaspiro[4.4]nonane-8-carboxamide (Compound 217)

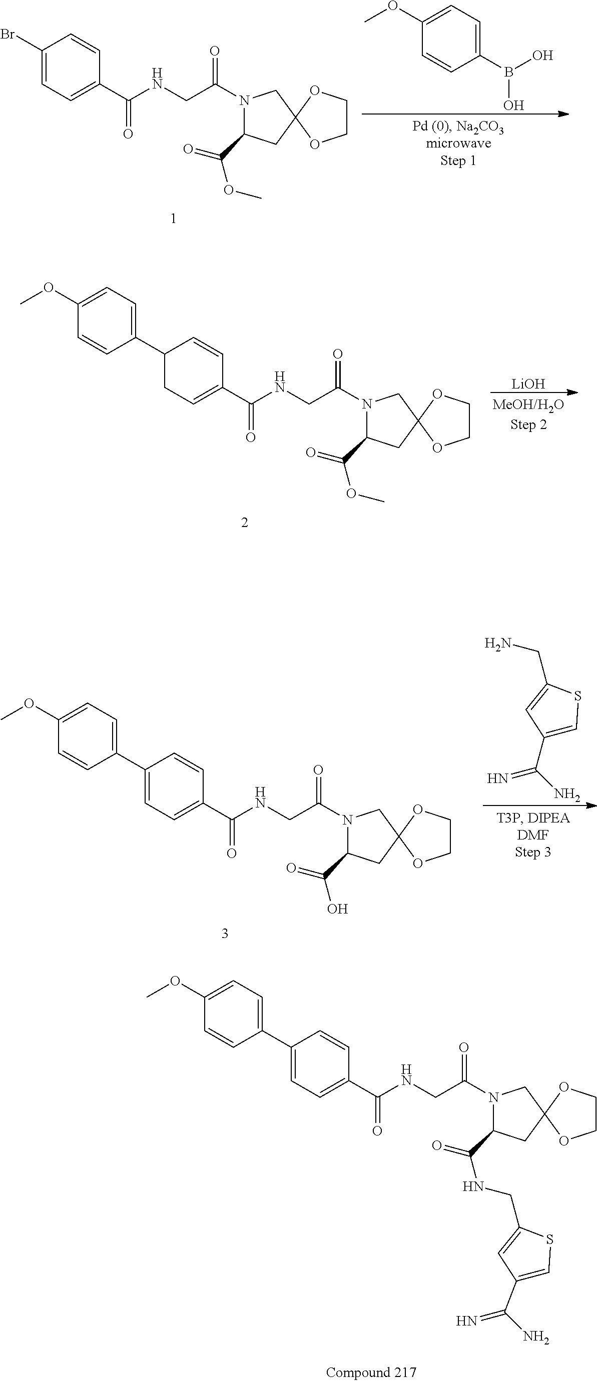

Compound 217 was prepared from (4-methoxyphenyl)boronic acid, methyl (8S)-7-[2-[(4-bromobenzoyl)amino]acetyl]-1,4-dioxa-7-azaspiro[4.4]nonane-8-carboxylate, and 5-(aminomethyl)thiophene-3-carboximidamide. HCl based on the procedures set forth in Scheme 199. LC/MS (ESI) m/z: 578 (M+H)⁺. ¹H NMR (400 MHz, DMSO) δ 8.69 (s, 1H), 8.44 (s, 1H), 7.95-7.89 (m, 2H), 7.70 (d, J=9.3 Hz, 2H), 7.50 (s, 1H), 7.05 (d, J=7.7 Hz, 2H), 4.72-4.66 (m, 3H), 4.40-3.95 (m, 3H), 3.81 (s, 3H), 3.73-3.61 (m, 3H), 2.13-2.06 (m, 1H), 1.75-1.65 (m, 1H)

### Scheme 209: Synthesis of (2S,4S)—N—((R)-1-(4-carbamimidoylthiophen-2-yl)ethyl)-1-((dibenzo[b,d]furan-2-carbonyl)glycyl)-4-phenylpyrrolidine-2-carboxamide (Compound 218)

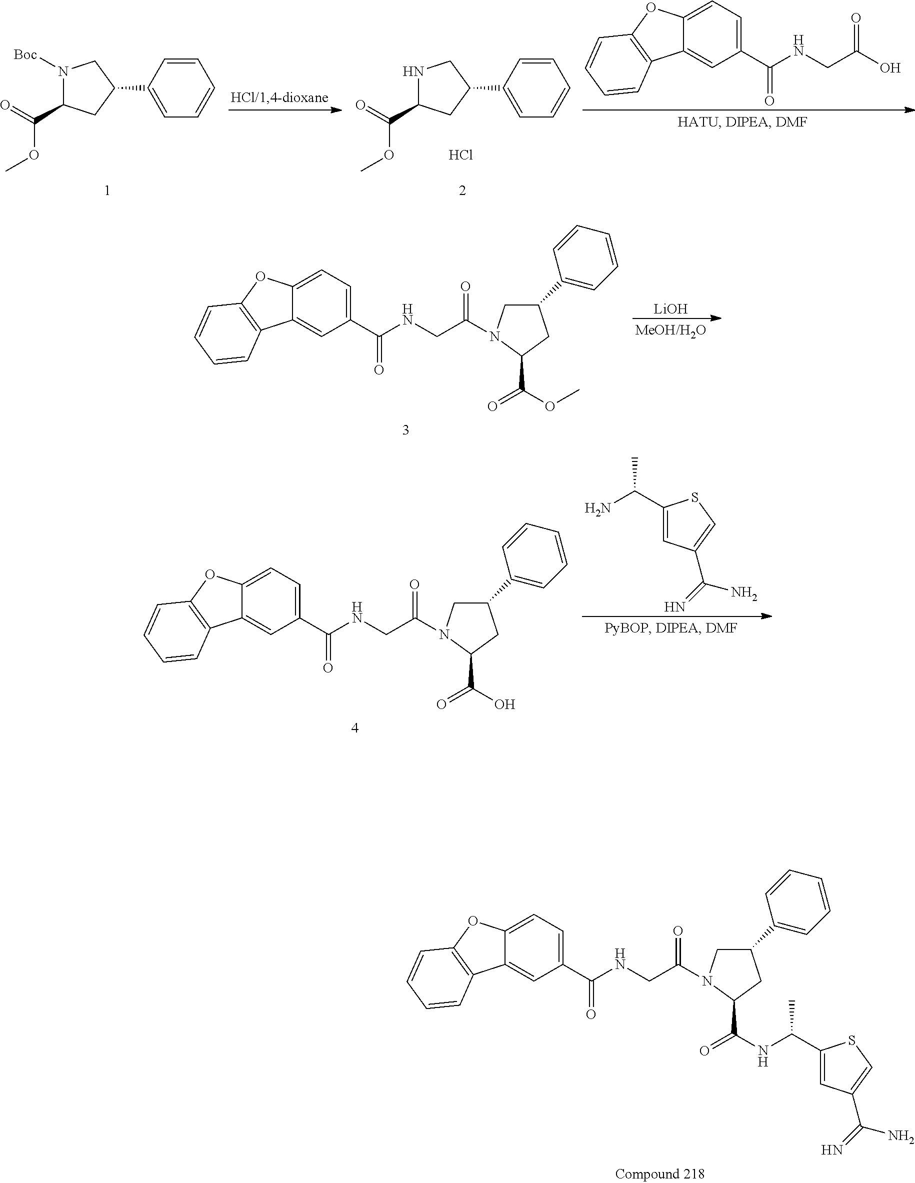

Compound 218 was prepared from 1-(tert-butyl) 2-methyl (2S,4S)-4-phenylpyrrolidine-1,2-dicarboxylate, (dibenzo[b,d]furan-2-carbonyl)glycine, and (R)-5-(1-aminoethyl)thiophene-3-carboximidamide based on the procedures set forth in Scheme 3. ¹H NMR (400 MHz, CD₃OD) δ 8.60 (s, 1H), 8.55 (s, 1H), 8.17 (s, 1H), 8.06 (dd, J=17.5, 8.1 Hz, 2H), 7.65 (dd, J=13.3, 8.5 Hz, 2H), 7.54 (dd, J=16.0, 8.2 Hz, 2H), 7.42 (t, J=7.2 Hz, 1H), 7.32 (dd, J=25.3, 10.4 Hz, 5H), 5.34-5.29 (m, 1H), 4.72 (d, J=6.5 Hz, 1H), 4.27 (d, J=26.5 Hz, 3H), 3.74 (d, J=4.2 Hz, 2H), 2.45 (d, J=9.9 Hz, 1H), 2.15 (dd, J=18.1, 10.6 Hz, 1H), 1.63 (t, J=17.3 Hz, 3H). LCMS (ESI) (m/z): 594 (M+H)⁺.

### Scheme 210: Synthesis of (2S,4S*)—N—((R)-1-(4-carbamimidoylthiophen-2-yl)ethyl)-1-((dibenzo[b,d]furan-2-carbonyl)glycyl)-4-(methoxymethyl)pyrrolidine-2-carboxamide (Compound 219)

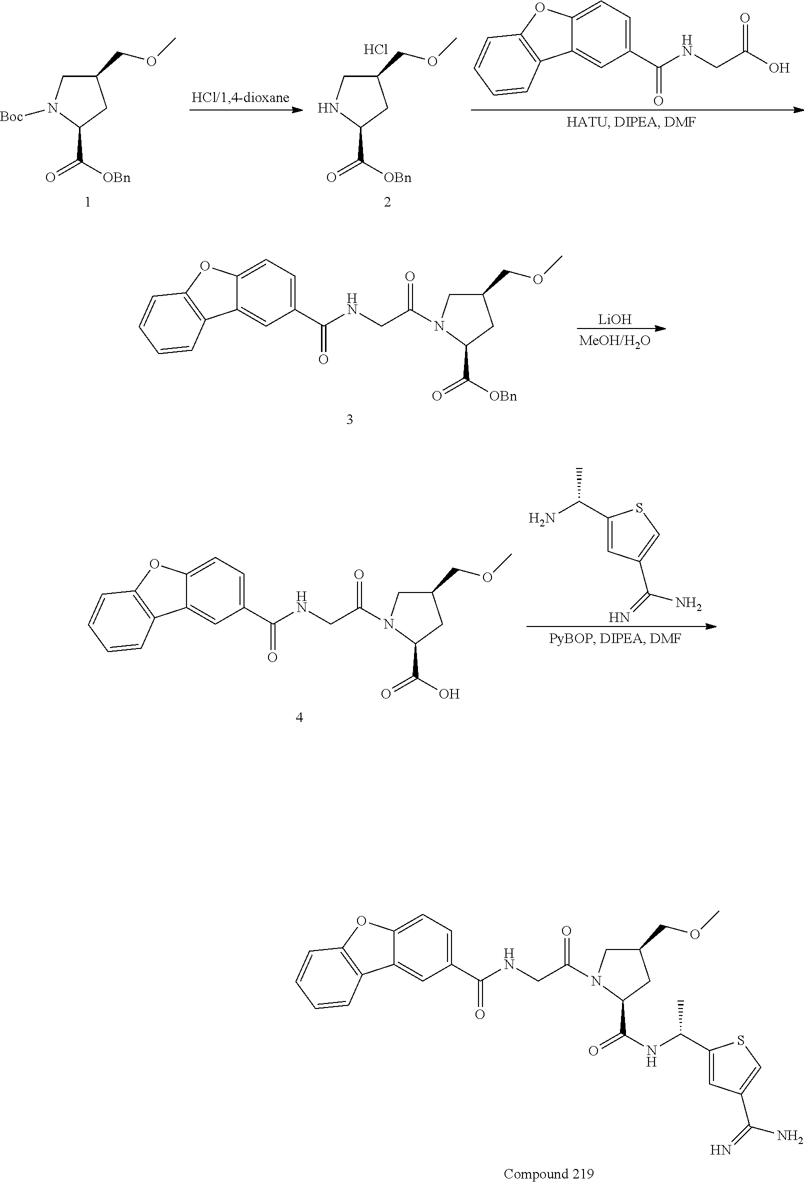

Compound 219 was prepared from 2-benzyl 1-(tert-butyl) (2S,4S*)-4-(methoxymethyl)pyrrolidine-1,2-dicarboxylate, (dibenzo[b,d]furan-2-carbonyl)glycine, and (R)-5-(1-aminoethyl)thiophene-3-carboximidamide based on the procedures set forth in Scheme 3. ¹H NMR (400 MHz, CD₃OD) δ 8.58 (s, 1H), 8.54 (s, 1H), 8.20 (d, J=1.4 Hz, 1H), 8.11-7.99 (m, 2H), 7.65 (dd, J=11.9, 8.4 Hz, 2H), 7.58-7.52 (m, 2H), 7.42 (t, J=7.4 Hz, 1H), 5.28 (d, J=7.3 Hz, 1H), 4.46 (t, J=8.1 Hz, 1H), 4.36 (d, J=16.7 Hz, 1H), 4.17 (d, J=16.7 Hz, 1H), 4.05-3.93 (m, 1H), 3.44 (dd, J=15.0, 7.7 Hz, 3H), 3.35 (s, 3H), 2.66 (d, J=7.7 Hz, 1H), 2.54-2.39 (m, 1H), 1.77 (dd, J=21.5, 9.2 Hz, 1H), 1.61 (t, J=16.5 Hz, 3H). LCMS (ESI) (m/z): 562 (M+H)⁺.

### Scheme 211: Synthesis of (2S,4R)—N—((R)-1-(4-carbamimidoylthiophen-2-yl)ethyl)-1-((dibenzo[b,d]furan-2-carbonyl)glycyl)-4-(methylsulfonyl)pyrrolidine-2-carboxamide (Compound 220)

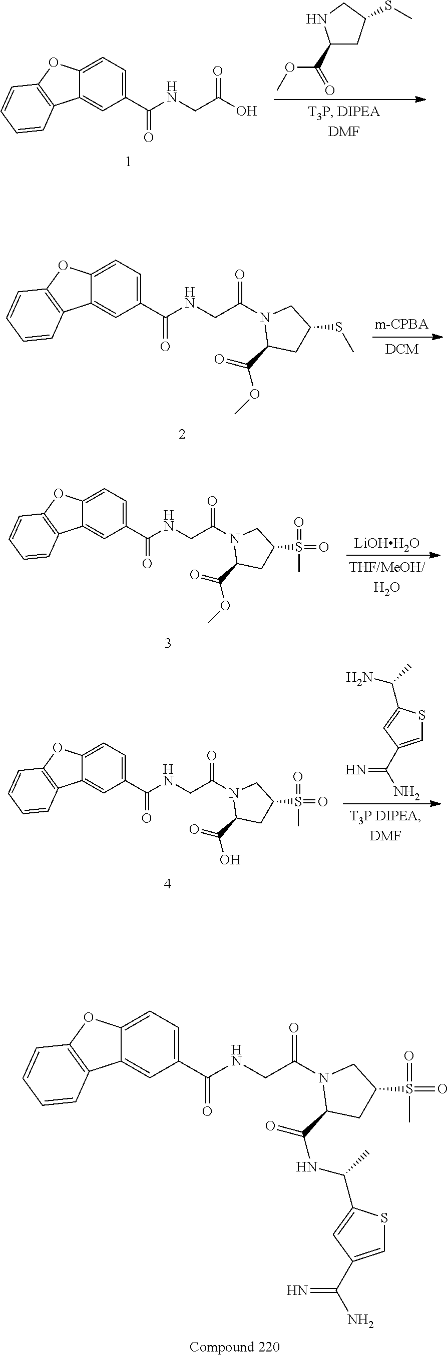

Compound 220 was prepared as a white solid from (dibenzo[b,d]furan-2-carbonyl)glycine, methyl (2S,4R)-4-(methylthio)pyrrolidine-2-carboxylate, and (R)-5-(1-aminoethyl)thiophene-3-carboximidamide based on the procedures set forth in Scheme 169. ¹H NMR (400 MHz, CD₃OD) δ 8.61-8.57 (m, 1H), 8.50 (s, 1H), 8.23 (dd, J=18.8, 1.1 Hz, 1H), 8.09 (t, J=8.1 Hz, 1H), 8.02 (td, J=8.9, 1.7 Hz, 1H), 7.66 (dd, J=12.9, 8.5 Hz, 2H), 7.54 (dd, J=16.9, 8.8 Hz, 2H), 7.43 (t, J=7.5 Hz, 1H), 5.28 (d, J=7.4 Hz, 1H), 4.73 (s, 1H), 4.33 (s, 2H), 4.20 (dd, J=17.8, 7.7 Hz, 3H), 3.08 (d, J=18.7 Hz, 3H), 2.82-2.74 (m, 1H), 2.47-2.41 (m, 1H), 1.61 (s, 3H). LC/MS (ESI) m/z: 596 (M+H)⁺.

### Scheme 212: Synthesis of (2S,4R)—N—((R)-1-(4-carbamimidoylthiophen-2-yl)ethyl)-1-((dibenzo[b,d]furan-2-carbonyl)glycyl)-4-methoxypyrrolidine-2-carboxamide (Compound 221)

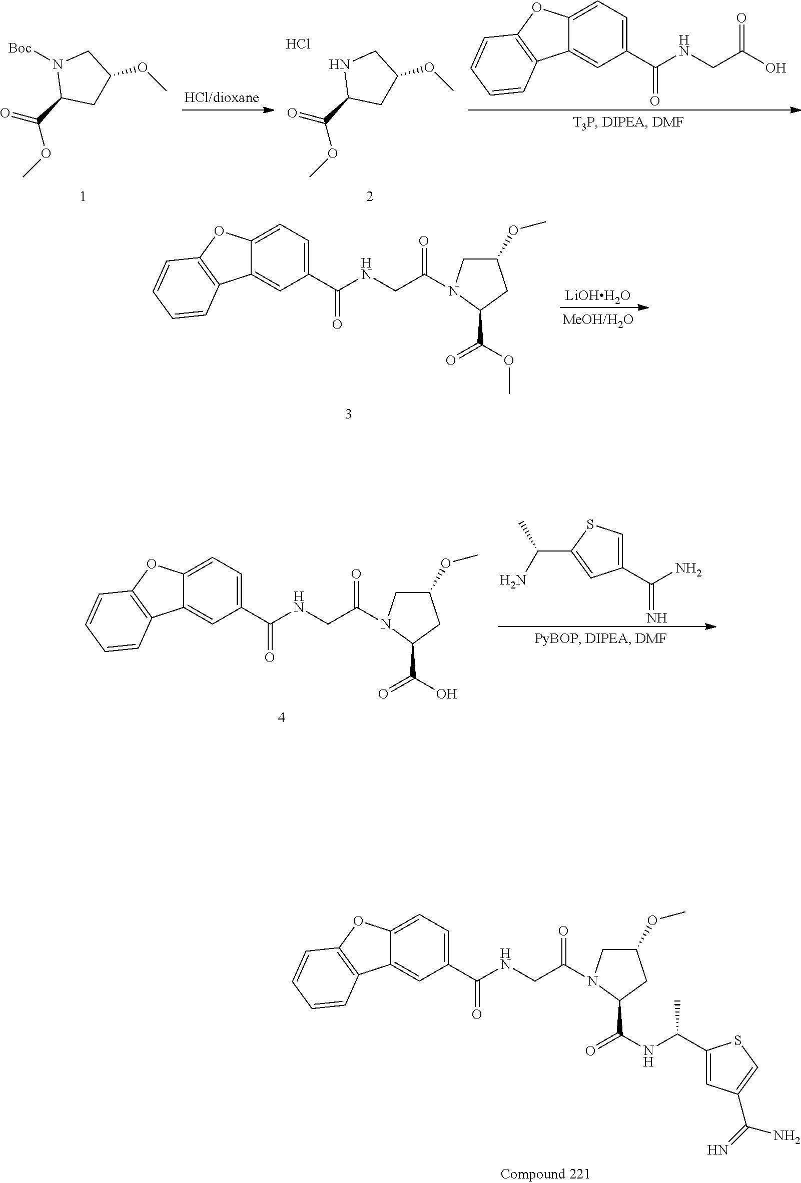

Compound 221 was prepared from 1-(tert-butyl) 2-methyl (2S,4R)-4-methoxypyrrolidine-1,2-dicarboxylate, (dibenzo[b,d]furan-2-carbonyl)glycine, and (R)-5-(1-aminoethyl)thiophene-3-carboximidamide based on procedures set forth in Scheme 3. ¹H NMR (400 MHz, CD₃OD) δ 8.58 (d, J=8.3 Hz, 1H), 8.53 (s, 1H), 8.23 (d, J=18.6 Hz, 1H), 8.08 (d, J=7.7 Hz, 1H), 8.05-8.00 (m, 1H), 7.66 (dd, J=11.6, 8.6 Hz, 2H), 7.55 (t, J=7.6 Hz, 2H), 7.43 (t, J=7.5 Hz, 1H), 5.35-5.21 (m, 1H), 4.51 (t, J=8.1 Hz, 1H), 4.35 (d, J=16.6 Hz, 1H), 4.22 (d, J=16.7 Hz, 1H), 3.84 (dt, J=11.3, 7.9 Hz, 2H), 3.40 (s, 3H), 3.35 (s, 1H), 2.49-2.37 (m, 1H), 2.07 (ddd, J=13.4, 8.6, 5.0 Hz, 1H), 1.61 (t, J=16.8 Hz, 3H). LC/MS (ESI) m/z: 548 (M+H)⁺.

### Scheme 213: Synthesis of (2S,4R)—N—((R)-1-(4-carbamimidoylthiophen-2-yl)ethyl)-1-((dibenzo[b,d]furan-2-carbonyl)glycyl)-4-fluoro-4-(methoxymethyl)pyrrolidine-2-carboxamide (Compound 222)

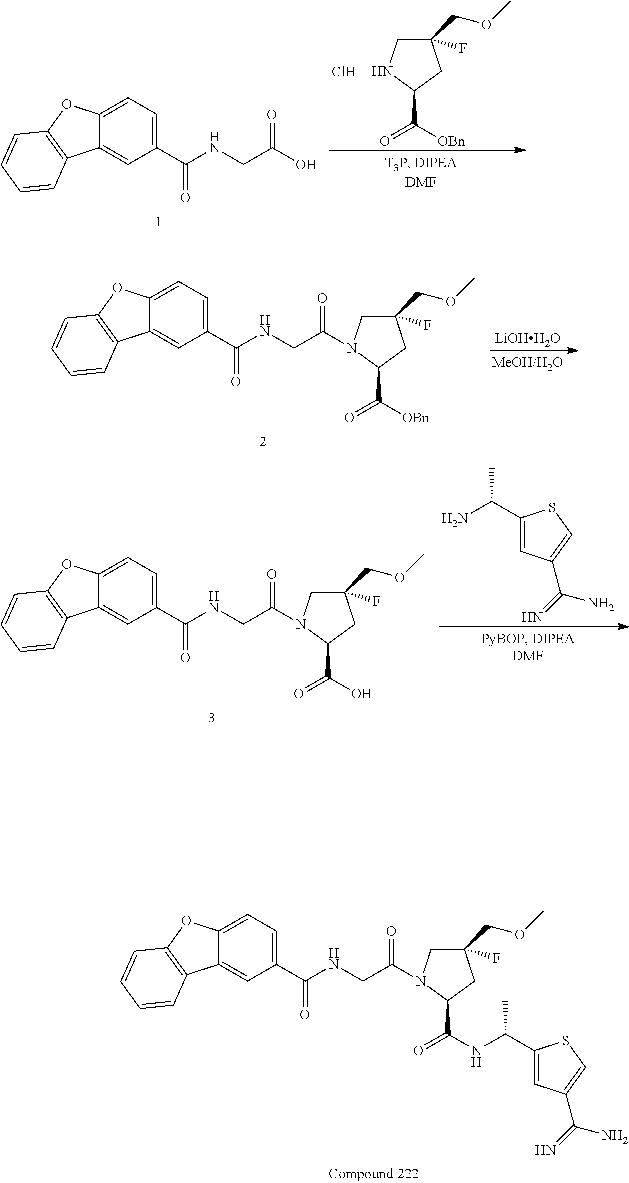

Compound 222 was prepared from (dibenzo[b,d]furan-2-carbonyl)glycine, benzyl (2S,4R)-4-fluoro-4-(methoxymethyl)pyrrolidine-2-carboxylate hydrochloride, and (R)-5-(1-aminoethyl)thiophene-3-carboximidamide based on the procedures set forth in Scheme 3. ¹H NMR (400 MHz, CD₃OD) δ 8.54 (s, 1H), 8.27-8.16 (m, 1H), 8.10-8.00 (m, 2H), 7.70-7.63 (m, 2H), 7.58-7.53 (m, 2H), 7.43 (t, J=7.6 Hz, 1H), 5.32-5.24 (m, 1H), 4.66-4.61 (m, 1H), 4.42-4.33 (m, 1H), 4.22-4.07 (m, 2H), 3.99-3.87 (m, 1H), 3.75-3.68 (m, 2H), 3.44 (s, 3H), 2.80-2.41 (m, 1H), 2.28-2.13 (m, 1H), 1.59 (d, J=6.8 Hz, 3H). LC/MS (ESI) m/z: 580 (M+H)⁺.

### Scheme 214: Synthesis of (2S,4R)—N—((R)-1-(4-carbamimidoylthiophen-2-yl)ethyl)-1-((dibenzo[b,d]furan-2-carbonyl)glycyl)-4-fluoro-4-(fluoromethyl)pyrrolidine-2-carboxamide (Compound 223)

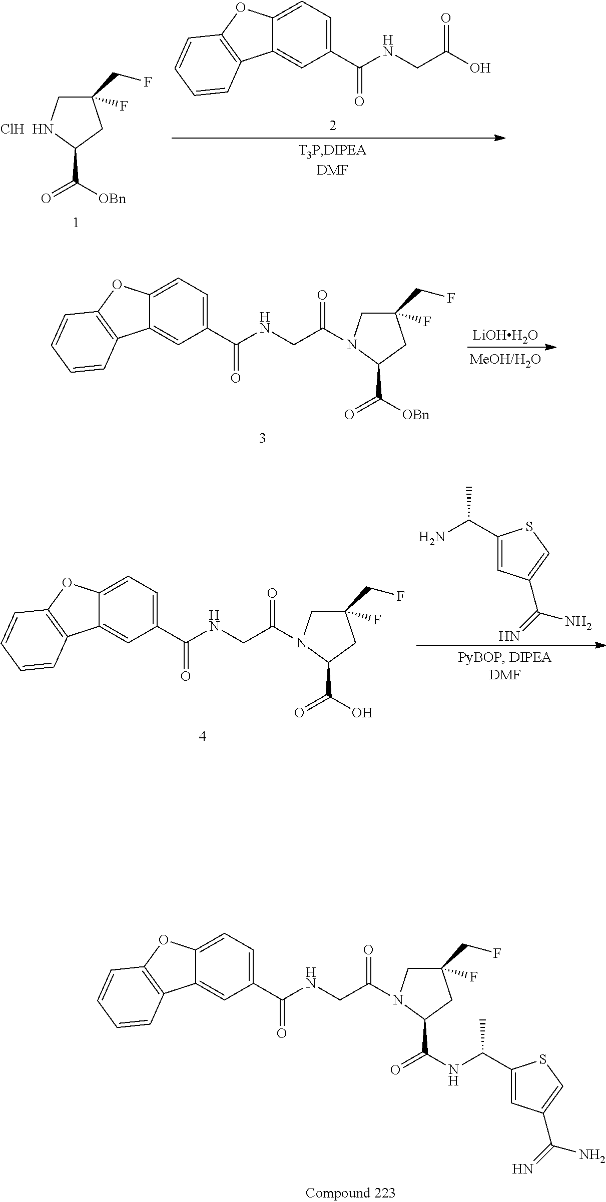

Compound 223 was prepared from benzyl (2S,4R)-4-fluoro-4-(fluoromethyl)pyrrolidine-2-carboxylate hydrochloride, (dibenzo[b,d]furan-2-carbonyl)glycine, and (R)-5-(1-aminoethyl)thiophene-3-carboximidamide based on the procedures set forth in Scheme 3. ¹H NMR (400 MHz, CD₃OD) δ 8.53 (s, 1H), 8.26-8.18 (m, 1H), 8.10-8.00 (m, 2H), 7.69-7.62 (m, 2H), 7.58-7.51 (m, 2H), 7.42 (t, J=7.4 Hz, 1H), 5.39-5.20 (m, 1H), 4.81 (d, J=3.3 Hz, 1H), 4.70-4.60 (m, 2H), 4.44-4.32 (m, 1H), 4.22-4.12 (m, 2H), 4.04-3.90 (m, 1H), 2.78-2.49 (m, 1H), 2.31-2.14 (m, 1H), 1.59 (d, J=6.8 Hz, 3H). LC/MS (ESI) m/z: 568 (M+H)⁺.

### Scheme 215: Synthesis of (2S,4R)-1-[2-(anthracen-2-ylformamido)acetyl]-N-[(1R)-1-(4-carbamimidoylthiophen-2-yl)ethyl]-4-fluoro-4-(methoxymethyl)pyrrolidine-2-carboxamide (Compound 224)

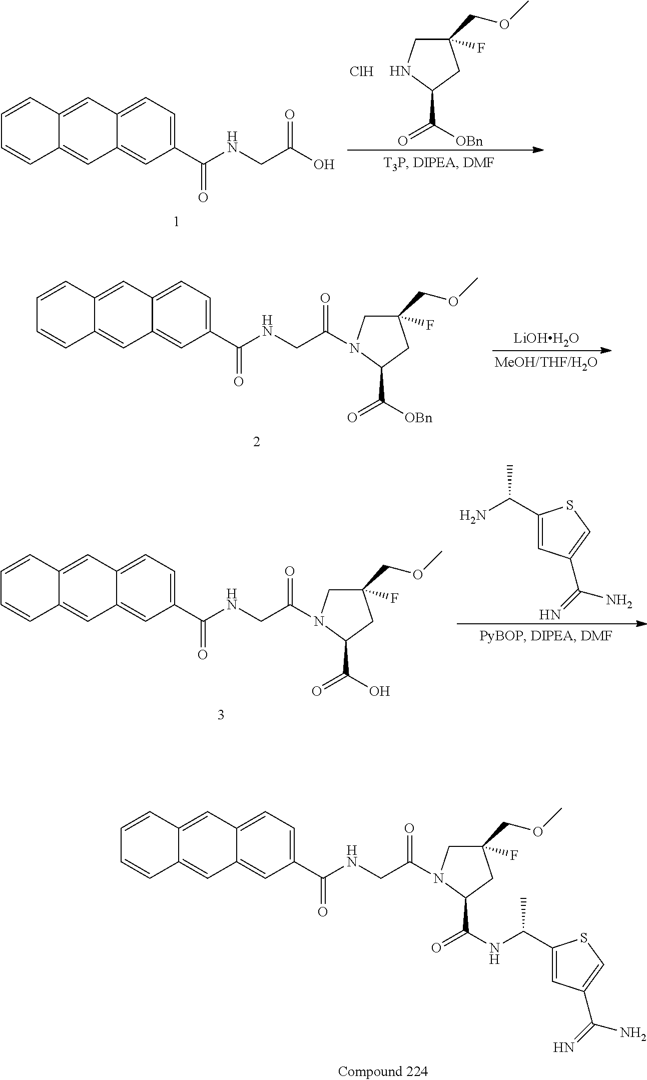

Compound 224 was prepared from (2S,4R)-1-(anthracen-2-ylformamido)acetic acid, benzyl (2S,4R)-4-fluoro-4-(methoxymethyl)pyrrolidine-2-carboxylate hydrochloride, and 5-[(1R)-1-aminoethyl]thiophene-3-carboximidamide based on the procedures set forth in Scheme 3. ¹H NMR (400 MHz, CD₃OD) δ 8.61 (d, J=5.6 Hz, 2H), 8.54 (s, 1H), 8.25-8.03 (m, 4H), 7.86 (d, J=9.6 Hz, 1H), 7.60-7.46 (m, 3H), 5.31-5.23 (m, 1H), 4.67-4.64 (m, 1H), 4.42-4.35 (m, 1H), 4.23-4.18 (m, 1H), 4.11-4.04 (m, 1H), 3.99-3.89 (m, 1H), 3.76-3.67 (m, 2H), 3.44 (s, 3H), 2.62-2.50 (m, 1H), 2.26-2.17 (m, 1H), 1.59 (d, J=6.8 Hz, 3H). LC/MS (ESI) m/z: 590 (M+H)⁺.

### Scheme 216: Synthesis of (S)—N—((R)-1-(4-carbamimidoylthiophen-2-yl)ethyl)-7-((6,9,9-trifluoro-9H-fluorene-3-carbonyl)glycyl)-1,4-dioxa-7-azaspiro[4.4]nonane-8-carboxamide (Compound 225)

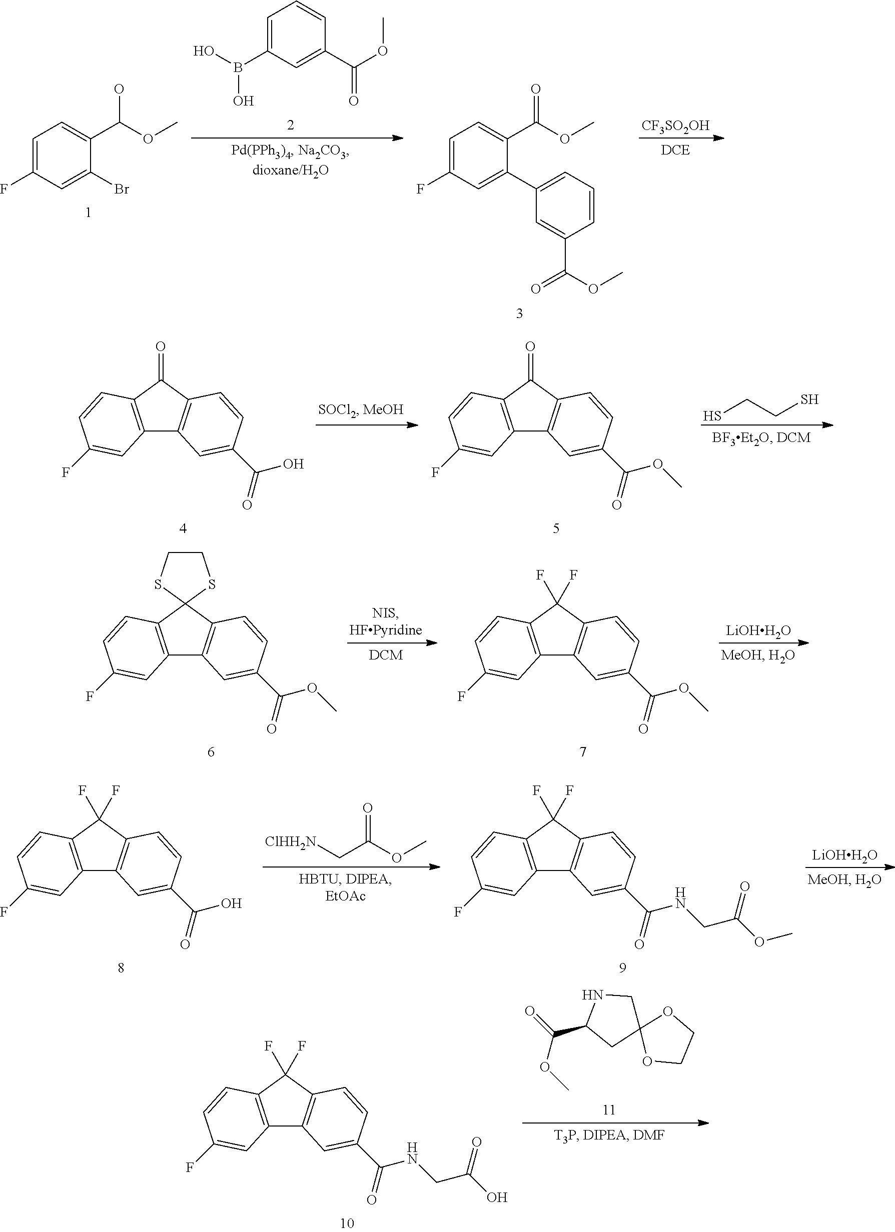

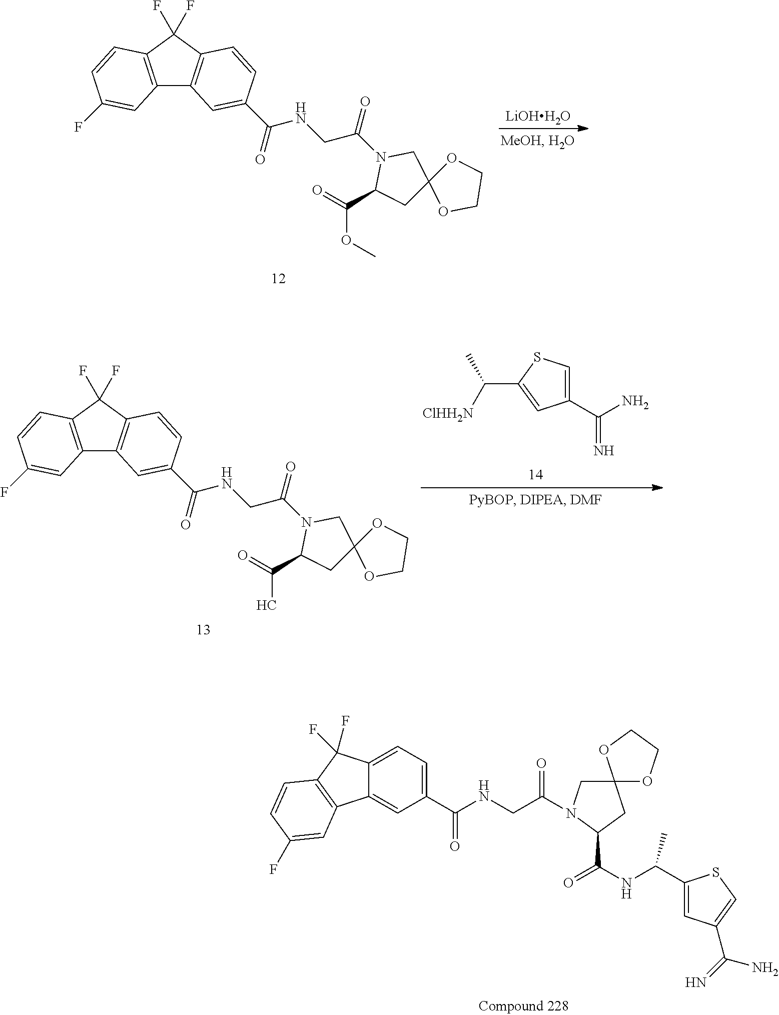

Compound 225 was prepared from methyl 2-bromo-4-fluorobenzoate, (3-(methoxycarbonyl)phenyl)boronic acid, methyl glycinate hydrochloride, methyl (S)-1,4-dioxa-7-azaspiro[4.4]nonane-8-carboxylate, and (R)-5-(1-aminoethyl)thiophene-3-carboximidamide hydrochloride based on the procedures set forth in Scheme 181. ¹H NMR (400 MHz, CD₃OD) δ 8.23 (t, J=12.3 Hz, 2H), 7.92 (d, J=7.3 Hz, 1H), 7.76-7.67 (m, 2H), 7.53 (d, J=15.4 Hz, 2H), 7.18 (s, 1H), 5.30 (s, 1H), 4.57 (s, 1H), 4.22 (d, J=7.1 Hz, 2H), 4.00 (d, J=8.7 Hz, 4H), 3.80 (d, J=7.4 Hz, 2H), 2.19 (s, 1H), 2.04 (s, 1H), 1.58 (d, J=7.0 Hz, 3H). LCMS (ESI) (m/z): 628 (M+H)⁺.

### Scheme 217: Synthesis of (2S,4S)—N—((R)-1-(4-carbamimidoylthiophen-2-yl)ethyl)-1-((dibenzo[b,d]furan-2-carbonyl)glycyl)-4-methylpyrrolidine-2-carboxamide (Compound 226)

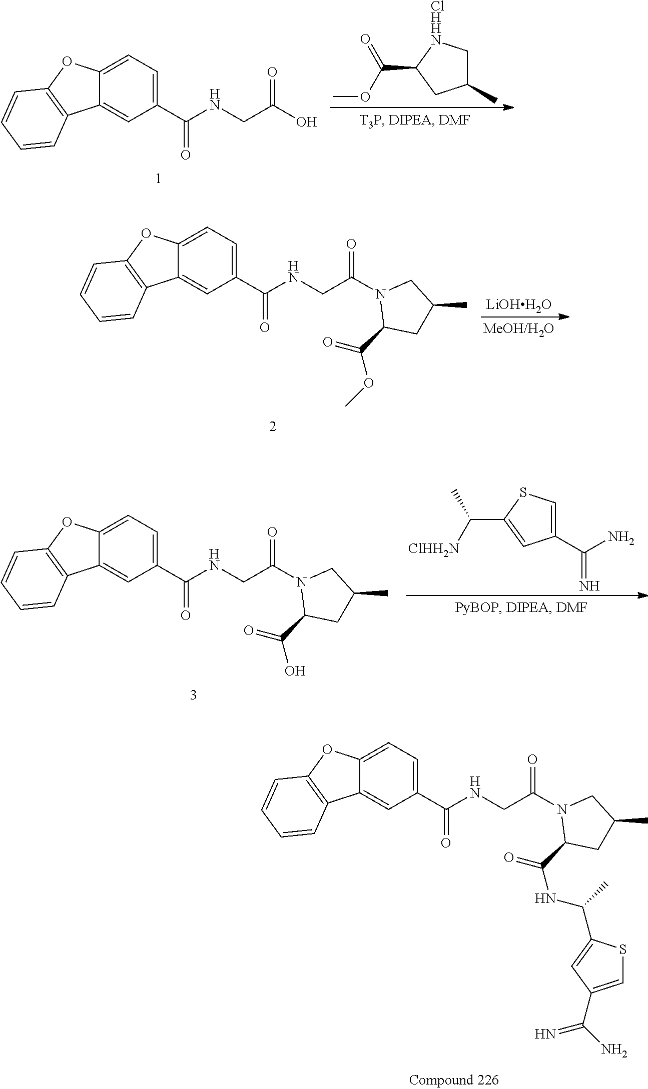

Compound 226 was prepared from (dibenzo[b,d]furan-2-carbonyl)glycine, methyl (2S,4S)-4-methylpyrrolidine-2-carboxylate hydrochloride, and (R)-5-(1-aminoethyl)thiophene-3-carboximidamide hydrochloride based on the procedures set forth in Scheme 3. ¹H NMR (400 MHz, CD₃OD) δ 8.57 (s, 1H), 8.21 (d, J=26.1 Hz, 1H), 8.04 (dd, J=18.2, 8.0 Hz, 2H), 7.67-7.61 (m, 2H), 7.57-7.50 (m, 2H), 7.42 (t, J=7.5 Hz, 1H), 5.27 (d, J=6.8 Hz, 1H), 4.48-4.30 (m, 2H), 4.15 (d, J=16.6 Hz, 1H), 4.05-3.90 (m, 1H), 3.22 (t, J=9.8 Hz, 1H), 2.55-2.34 (m, 2H), 1.57 (t, J=7.1 Hz, 4H), 1.14 (t, J=10.3 Hz, 3H). LCMS (ESI) (m/z): 532 (M+H)⁺.

### Scheme 218. (S)-4′-((2-(8-(((4-carbamimidoylthiophen-2-yl)methyl)carbamoyl)-1,4-dioxa-7-azaspiro[4.4]nonan-7-yl)-2-oxoethyl)carbamoyl)-4-fluoro-[1,1′-biphenyl]-2-carboxylic acid (Compound 227)

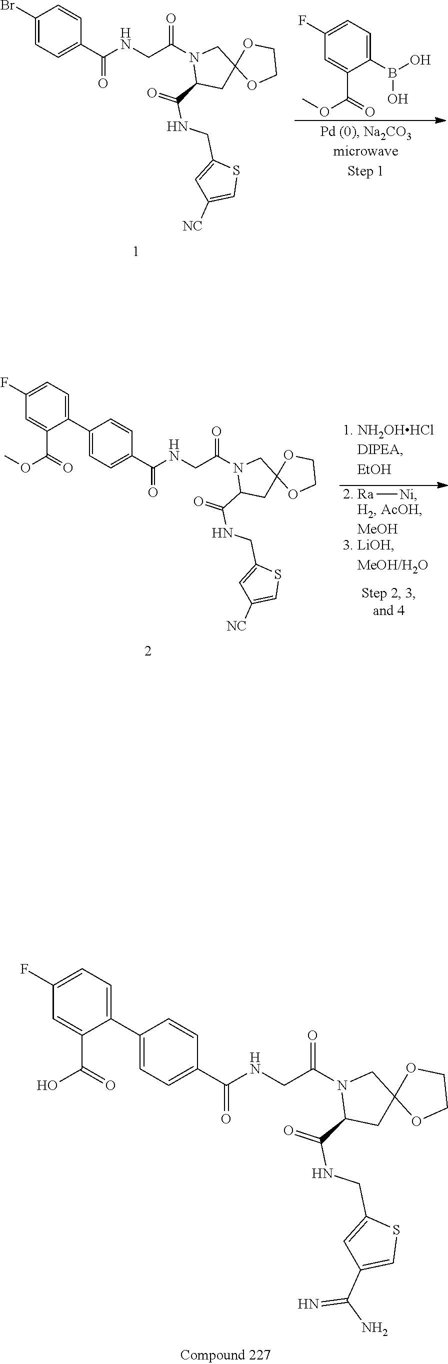

Step 1: methyl (S)-4′-((2-(8-(((4-cyanothiophen-2-yl)methyl)carbamoyl)-1,4-dioxa-7-azaspiro[4.4]nonan-7-yl)-2-oxoethyl)carbamoyl)-4-fluoro-[1,1′-biphenyl]-2-carboxylate (2) A mixture of (4-fluoro-2-methoxycarbonyl-phenyl)boronic acid (20.04 mg, 0.1012 mmol, 1.2 equiv.), (8S)-7-[2-[(4-bromobenzoyl)amino]acetyl]-N-[(5-cyano-3-thienyl)methyl]-1,4-dioxa-7-azaspiro[4.4]nonane-8-carboxamide (0.045 g, 0.084 mmol, 1 equiv.), Pd(dppf)Cl₂—CH₂Cl₂ (7.03 mg, 0.0084 mmol, 0.1 equiv.), and Na₂CO₃ (17.88 mg, 0.169 mmol, 2 equiv.) in 1,4-dioxane (3 mL) and water (0.3 mL) was stirred in a microwave reactor at 110° C. for 30 min. The reaction was cooled to room temperature and then filtered through Celite pad. The filtrate was diluted with water and EtOAc. The two layers were separated, and the aqueous layer was extracted with EtOAc (5 mL×2). The combined organic extracts were washed with brine, dried over Na₂SO₄ and concentrated. Purification by combi-Flash; 4 g column, solvent A=CH₂Cl₂, solvent B=MeOH; 100% A to 5% B gave methyl 2-[4-[[2-[(8S)-8-[2-(4-cyano-2-thienyl)acetyl]-1,4-dioxa-7-azaspiro[4.4]nonan-7-yl]-2-oxo-ethyl]carbamoyl]phenyl]-5-fluoro-benzoate (35.00 mg, 0.05916 mmol, 70.13% yield). LC/MS (ESI) m/z: 592 (M+H)⁺.

### Steps 2,3, and 4: (S)-4′-((2-(8-(((4-carbamimidoylthiophen-2-yl)methyl)carbamoyl)-1,4-dioxa-7-azaspiro[4.4]nonan-7-yl)-2-oxoethyl)carbamoyl)-4-fluoro-[1,1′-biphenyl]-2-carboxylic acid (Compound 227)

To a solution of methyl 2-[4-[[2-[(8S)-8-[2-(4-cyano-2-thienyl)acetyl]-1,4-dioxa-7-azaspiro[4.4]nonan-7-yl]-2-oxo-ethyl]carbamoyl]phenyl]-5-fluoro-benzoate (30.00 mg, 0.05071 mmol, 1 equiv.) in EtOH (2 mL) was added NH₂OH·HCl (8.900 mg, 0.127 mmol, 2.5 equiv.), and DIPEA (26.50 μL, 0.152 mmol, 3 equiv.). The reaction was heated to 90° C. and stirred overnight. The solvent was removed under Rota vamp evaporation. Water (5 mL) and CH₂Cl₂ (5 mL) were added to the residue. The two layers were separated, and the aqueous layer was extracted twice with CH₂Cl₂. The combined organic extracts were washed with brine, dried over Na₂SO₄ and concentered. To a solution of the residue obtained from the previous step in MeOH (1 mL) was added Ra—Ni (50 mass %) in H₂O (1.0 mg, 0.00584 mmol, 0.1 equiv.), and AcOH (10 μL). The flask was evacuated and backfilled with H₂ in a balloon. The reaction was stirred at room temperature for 3 hours. The mixture then filtered through Celite pad. The filtrate was concentrated under reduced pressure to give a brown residue. LiOH (1.457 mg, 0.06084 mmol, 1.2 equiv.) was added to the residue above in MeOH (1.5 mL) and water (0.5 mL). The reaction was stirred at room temperature for 4 hours and then to remove MeOH. The pH was adjusted to 7 using 1 N HCl. Purification by HPLC to give Compound 227 (8.000 mg, 0.01312 mmol, 25.88% yield). LC/MS (ESI) m/z: 610 (M+H)⁺. ¹H NMR (400 MHz, DMSO) δ 10.42 (s, 1H), 8.53 (d, J=6.6 Hz, 1H), 8.29 (t, J=5.6 Hz, 1H), 8.16 (s, 1H), 7.72 (d, J=8.0 Hz, 1H), 7.51-7.37 (m, 2H), 7.31 (dd, J=8.8, 5.7 Hz, 1H), 7.08 (dd, J=8.9, 6.7 Hz, 1H), 4.51 (dd, J=16.3, 6.7 Hz, 2H), 4.36-4.11 (m, 1H), 4.07-3.77 (m, 4H), 3.70 (d, J=10.6 Hz, 2H), 3.54 (d, J=10.7 Hz, 2H), 2.22 (dd, J=12.9, 8.0 Hz, 1H), 1.99 (dd, J=12.9, 8.7 Hz, 1H).

### Scheme 219. (S)—N-((4-carbamimidoylthiophen-2-yl)methyl)-7-(2-(5-(2,4-difluorophenyl)-1-oxoisoindolin-2-yl)acetyl)-1,4-dioxa-7-azaspiro[4.4]nonane-8-carboxamide (Compound 228)

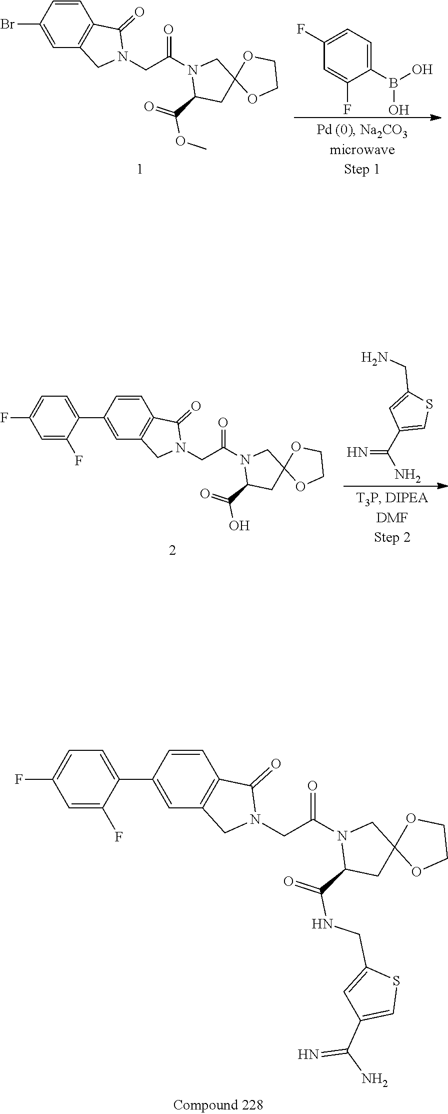 [SMILES: COC(=O)[C@@H]1CC2(CN1C(=O)CN1Cc3cc(Br)ccc3C1=O)OCCO2 ; OB(O)c1ccc(F)cc1F ; O=C(O)[C@@H]1CC2(CN1C(=O)CN1Cc3cc(-c4ccc(F)cc4F)ccc3C1=O)OCCO2 ; N=C(N)c1csc(CN)c1 ; N=C(N)c1csc(CNC(=O)[C@@H]2CC3(CN2C(=O)CN2Cc4cc(-c5ccc(F)cc5F)ccc4C2=O)OCCO3)c1]

Compound 228 was prepared from (2,4-difluorophenyl)boronic acid, methyl (8S)-7-[2-(5-bromo-1-oxo-isoindolin-2-yl)acetyl]-1,4-dioxa-7-azaspiro[4.4]nonane-8-carboxylate, and 5-(aminomethyl)thiophene-3-carboximidamide. HCl based on the procedures set forth in Scheme 206. LC/MS (ESI) m/z: 596 (M+H)⁺. ¹H NMR (400 MHz, DMSO) δ 8.80 (s, 1H), 8.36 (s, 1H), 7.84-7.69 (m, 1H), 7.62 (d, J=8.0 Hz, 2H), 7.39 (t, J=10.2 Hz, 2H), 7.23 (s, 1H), 4.66-4.19 (m, 3H), 4.07-3.70 (m, 2H), 3.66-3.10 (m, 6H), 2.38-1.86 (m, 2H), 0.96 (d, J=6.3 Hz, 1H), 0.87 (dd, J=15.7, 7.9 Hz, 1H).

### Scheme 220. (S)—N-((4-carbamimidoylthiophen-2-yl)methyl)-7-(2-(6-(2,4-difluorophenyl)-1-oxoisoindolin-2-yl)acetyl)-1,4-dioxa-7-azaspiro[4.4]nonane-8-carboxamide (Compound 229)

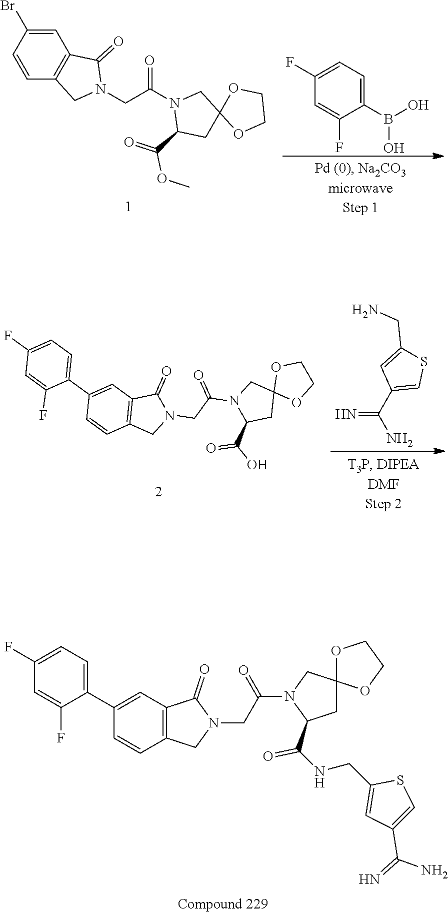 [SMILES: COC(=O)[C@@H]1CC2(CN1C(=O)CN1Cc3ccc(Br)cc3C1=O)OCCO2 ; OB(O)c1ccc(F)cc1F ; O=C(O)[C@@H]1CC2(CN1C(=O)CN1Cc3ccc(-c4ccc(F)cc4F)cc3C1=O)OCCO2 ; N=C(N)c1csc(CN)c1 ; N=C(N)c1csc(CNC(=O)[C@@H]2CC3(CN2C(=O)CN2Cc4ccc(-c5ccc(F)cc5F)cc4C2=O)OCCO3)c1]

Compound 229 was prepared from (2,4-difluorophenyl)boronic acid, methyl (8S)-7-[2-(6-bromo-1-oxo-isoindolin-2-yl)acetyl]-1,4-dioxa-7-azaspiro[4.4]nonane-8-carboxylate, and 5-(aminomethyl)thiophene-3-carboximidamide. HCl based on the procedures set forth in Scheme 206. LC/MS (ESI) m/z:596 (M+H)⁺. ¹H NMR (400 MHz, DMSO) δ 8.70 (s, 1H), 8.36 (s, 1H), 7.79-7.67 (m, 2H), 7.57 (d, J=8.2 Hz, 2H), 7.34 (t, J=10.3 Hz, 2H), 7.18 (s, 1H), 4.64-4.13 (m, 5H), 3.95-3.77 (m, 3H), 3.73 (d, J=10.5 Hz, 2H), 3.57 (t, J=14.7 Hz, 2H), 2.43 (p, J=1.9 Hz, 2H), 2.26-1.96 (m, 1H), 1.28-1.12 (m, 1H).

### Scheme 221. (S)—N-((4-carbamimidoylthiophen-2-yl)methyl)-7-(2-(6-(2,4-difluorophenyl)-1-oxoisoindolin-2-yl)acetyl)-1,4-dioxa-7-azaspiro[4.4]nonane-8-carboxamide (Compound 230)

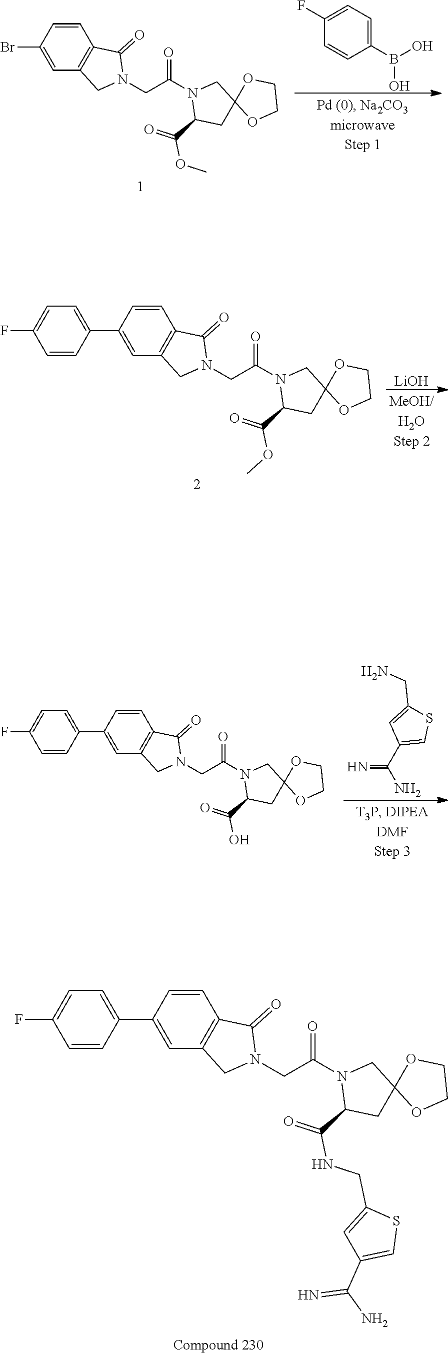

Compound 230 was prepared from (4-fluorophenyl)boronic acid, methyl (8S)-7-[2-(5-bromo-1-oxo-isoindolin-2-yl)acetyl]-1,4-dioxa-7-azaspiro[4.4]nonane-8-carboxylate, and 5-(aminomethyl)thiophene-3-carboximidamide. HCl based on the procedures set forth in Scheme 199. LC/MS (ESI) m/z:578 (M+H)⁺. ¹H NMR (400 MHz, DMSO) δ 8.71 (s, 1H), 8.38 (s, 1H), 7.87 (s, 1H), 7.82-7.73 (m, 3H), 7.33 (t, J=8.6 Hz, 2H), 7.11 (s, 1H), 4.49 (d, J=16.6 Hz, 2H), 4.45-4.34 (m, 3H), 3.98-3.85 (m, 3H), 3.80 (d, J=10.8 Hz, 1H), 3.70-3.57 (m, 3H), 2.99 (p, J=6.6 Hz, 2H), 2.45 (t, J=7.1 Hz, 2H), 2.32 (t, J=10.9 Hz, 1H), 2.04 (dd, J=12.9, 6.9 Hz, 1H).

### Scheme 222. (S)—N-((4-carbamimidoylthiophen-2-yl)methyl)-7-(2-(1-oxo-5-phenylisoindolin-2-yl)acetyl)-1,4-dioxa-7-azaspiro[4.4]nonane-8-carboxamide (Compound 231)

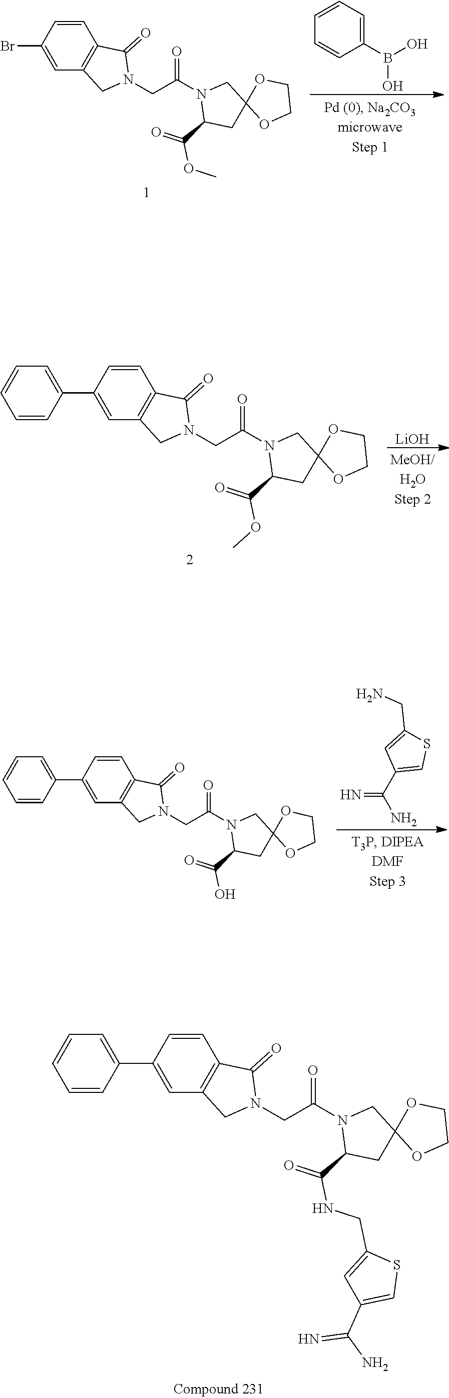

Compound 231 was prepared from phenylboronic acid, methyl (8S)-7-[2-(5-bromo-1-oxo-isoindolin-2-yl)acetyl]-1,4-dioxa-7-azaspiro[4.4]nonane-8-carboxylate, and 5-(aminomethyl)thiophene-3-carboximidamide. HCl based on the procedures set forth in Scheme 199. LC/MS (ESI) m/z: 560 (M+H)⁺. ¹H NMR (400 MHz, DMSO) δ 9.00 (s, 1H), 8.65 (s, 1H), 8.36 (s, 1H), 7.83 (d, J=7.6 Hz, 2H), 7.72 (d, J=4.4 Hz, 1H), 7.68 (d, J=7.8 Hz, 1H), 7.45 (t, J=7.5 Hz, 1H), 7.36 (t, J=7.3 Hz, 1H), 7.03 (s, 1H), 4.77-4.06 (m, 3H), 4.06-3.80 (m, 4H), 3.74 (d, J=10.8 Hz, 3H), 3.66-3.48 (m, 4H), 2.26 (dd, J=13.2, 8.8 Hz, 1H), 1.98 (dd, J=13.1, 6.9 Hz, 1H).

### Scheme 223: Synthesis of methyl ((E)-amino(5-((R)-1-((2S,4R)-1-((9,9-difluoro-9H-fluorene-3-carbonyl)glycyl)-4-fluoro-4-(methoxymethyl)pyrrolidine-2-carboxamido)ethyl)thiophen-3-yl)methylene)carbamate (Compound 232)

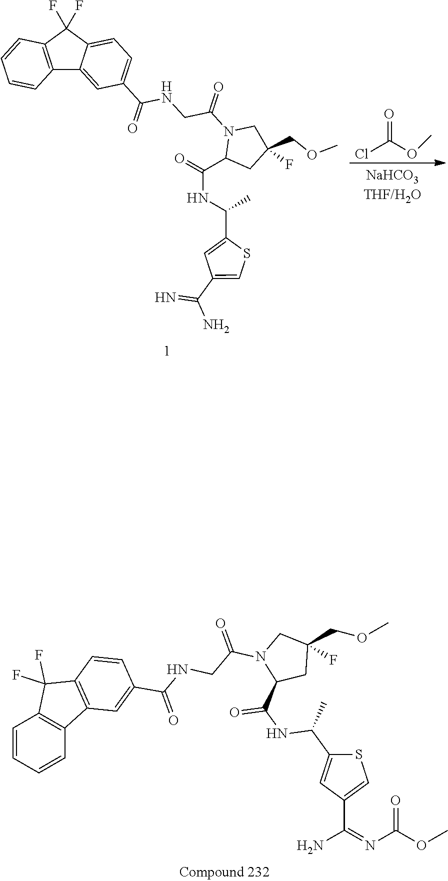

Compound 232 was prepared as a white solid from (2S,4R)—N—((R)-1-(4-carbamimidoylthiophen-2-yl)ethyl)-1-((9,9-difluoro-9H-fluorene-3-carbonyl)glycyl)-4-fluoro-4-(methoxymethyl)pyrrolidine-2-carboxamide and methyl carbonochloridate based on the procedures set forth in Scheme 143. ¹H NMR (400 MHz, CD₃OD) δ 8.16 (d, J=8.3 Hz, 1H), 8.06 (d, J=15.8 Hz, 1H), 7.86 (t, J=8.8 Hz, 1H), 7.80-7.68 (m, 3H), 7.64 (d, J=7.2 Hz, 1H), 7.59-7.53 (m, 1H), 7.43 (t, J=7.5 Hz, 1H), 5.23 (q, J=6.7 Hz, 1H), 4.66 (t, J=8.6 Hz, 1H), 4.38 (d, J=16.8 Hz, 1H), 4.15-3.95 (m, 2H), 3.78-3.63 (m, 3H), 3.46 (t, J=19.2 Hz, 6H), 2.55 (td, J=15.5, 7.9 Hz, 1H), 2.20 (ddd, J=36.7, 13.9, 9.4 Hz, 1H), 1.56 (d, J=7.0 Hz, 3H). LC/MS (ESI) (m/z): 672 (M+H)⁺.

### Scheme 224: Synthesis of methyl ((E)-amino(5-((R)-1-((2S,4R)-4-fluoro-4-(fluoromethyl)-1-((4-(4-fluorophenoxy)benzoyl)glycyl)pyrrolidine-2-carboxamido)ethyl)thiophen-3-yl)methylene)carbamate (Compound 233)

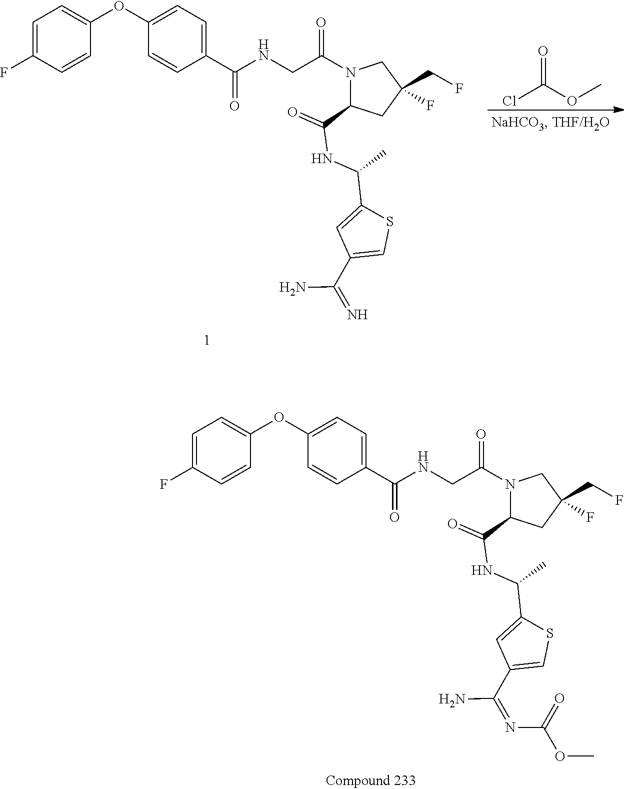

Compound 233 was prepared as a white solid from (2S,4R)—N—((R)-1-(4-carbamimidoylthiophen-2-yl)ethyl)-4-fluoro-4-(fluoromethyl)-1-((4-(4-fluorophenoxy)benzoyl)glycyl)pyrrolidine-2-carboxamide and methyl carbonochloridate based on the procedures set forth in Scheme 143. ¹H NMR (400 MHz, CD₃OD) δ 8.08 (s, 1H), 7.85 (d, J=8.8 Hz, 2H), 7.62 (d, J=58.6 Hz, 1H), 7.18-7.10 (m, 4H), 7.00 (t, J=9.9 Hz, 2H), 5.33-5.19 (m, 1H), 4.80-4.75 (m, 1H), 4.66 (dd, J=17.6, 7.7 Hz, 2H), 4.32 (d, J=16.8 Hz, 1H), 4.11 (dd, J=17.7, 10.2 Hz, 2H), 3.94 (dd, J=32.1, 12.2 Hz, 1H), 3.69 (s, 3H), 2.60 (td, J=15.5, 7.0 Hz, 1H), 2.21 (ddd, J=35.2, 14.3, 9.5 Hz, 1H), 1.56 (d, J=7.0 Hz, 3H). LCMS (ESI) m/z: 646 (M+H)⁺.

### Scheme 225: Synthesis of hexyl ((E)-amino(5-((R)-1-((2S,4R)-4-fluoro-4-(fluoromethyl)-1-((4-(4-fluorophenoxy)benzoyl)glycyl)pyrrolidine-2-carboxamido)ethyl)thiophen-3-yl)methylene)carbamate (Compound 234)

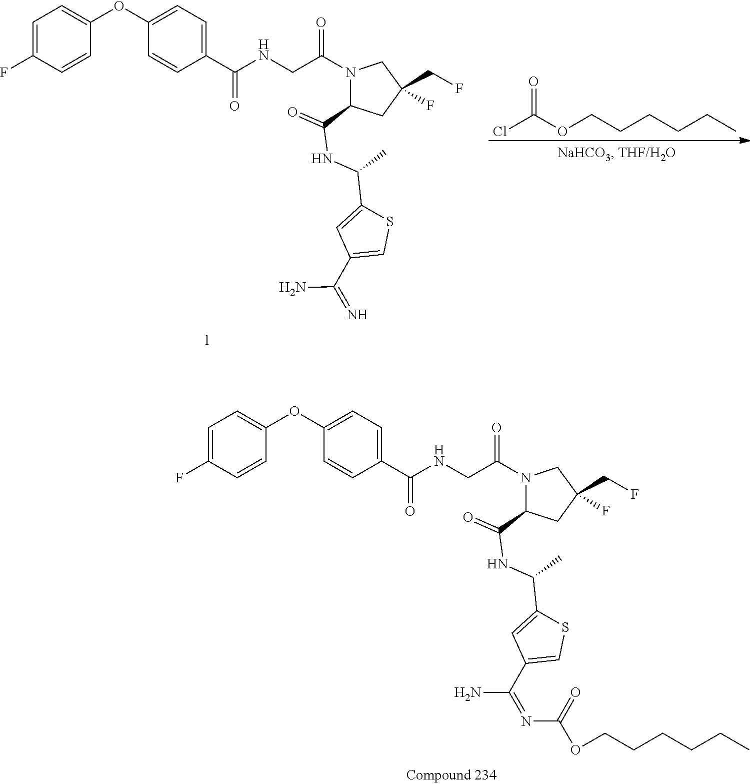

Compound 234 was prepared from (2S,4R)—N—((R)-1-(4-carbamimidoylthiophen-2-yl)ethyl)-4-fluoro-4-(fluoromethyl)-1-((4-(4-fluorophenoxy)benzoyl)glycyl)pyrrolidine-2-carboxamide and hexyl carbonochloridate based on the procedures set forth in Scheme 143. ¹H NMR (400 MHz, CD₃OD) δ 8.09 (s, 1H), 7.86 (d, J=8.8 Hz, 2H), 7.63 (d, J=63.8 Hz, 1H), 7.14 (t, J=6.2 Hz, 4H), 7.02 (d, J=8.8 Hz, 2H), 5.22 (d, J=6.5 Hz, 1H), 4.79 (d, J=7.1 Hz, 1H), 4.67 (dd, J=17.8, 8.6 Hz, 2H), 4.33 (d, J=16.8 Hz, 1H), 4.13-4.04 (m, 4H), 3.94 (dd, J=32.2, 12.5 Hz, 1H), 2.66-2.53 (m, 1H), 2.21 (ddd, J=35.1, 14.0, 8.9 Hz, 1H), 1.68-1.64 (m, 2H), 1.56 (d, J=7.0 Hz, 3H), 1.33 (s, 6H), 0.91 (t, J=6.8 Hz, 3H). LCMS (ESI) m/z: 716 (M+H)⁺. ¹H NMR (400 MHz, CD₃OD) δ 8.09 (s, 1H), 7.86 (d, J=8.8 Hz, 2H), 7.63 (d, J=63.8 Hz, 1H), 7.14 (t, J=6.2 Hz, 4H), 7.02 (d, J=8.8 Hz, 2H), 5.22 (d, J=6.5 Hz, 1H), 4.79 (d, J=7.1 Hz, 1H), 4.67 (dd, J=17.8, 8.6 Hz, 2H), 4.33 (d, J=16.8 Hz, 1H), 4.13-4.04 (m, 4H), 3.94 (dd, J=32.2, 12.5 Hz, 1H), 2.66-2.53 (m, 1H), 2.21 (ddd, J=35.1, 14.0, 8.9 Hz, 1H), 1.68-1.64 (m, 2H), 1.56 (d, J=7.0 Hz, 3H), 1.39-1.30 (m, 6H), 0.91 (t, J=6.8 Hz, 3H). LCMS (ESI) m/z: 716 (M+H)⁺.

### Scheme 226: Synthesis of (2S,4R)-1-((anthracene-2-carbonyl)glycyl)-N—((R)-1-(4-carbamimidoylthiophen-2-yl)ethyl)-4-fluoro-4-(fluoromethyl)pyrrolidine-2-carboxamide (Compound 235)

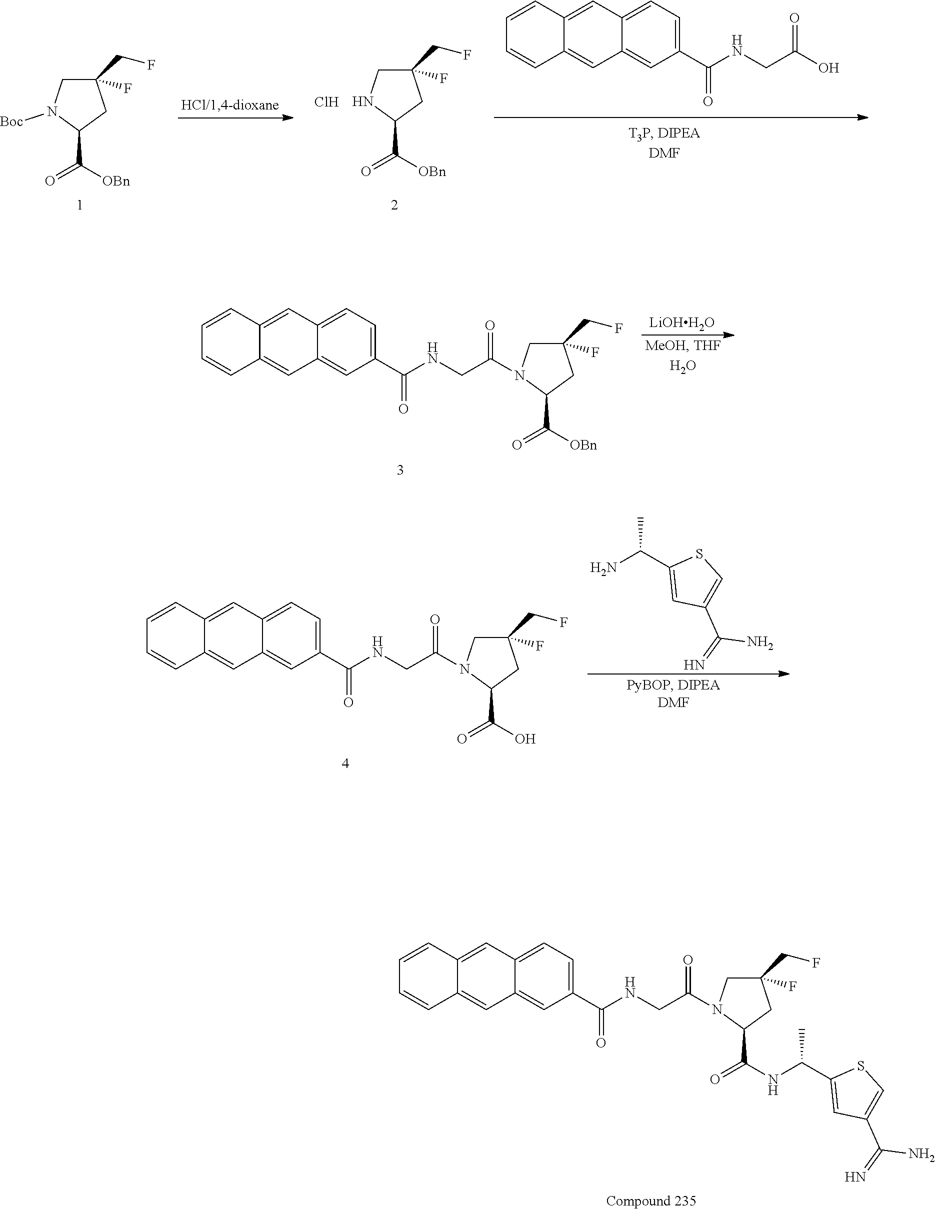

Compound 235 was prepared from 2-benzyl 1-(tert-butyl) (2S,4R)-4-fluoro-4-(fluoromethyl)pyrrolidine-1,2-dicarboxylate, (anthracene-2-carbony)glycine, and (R)-5-(1-aminoethyl)thiophene-3-carboximidamide based on the procedures set forth in Scheme 3. ¹H NMR (400 MHz, CD₃OD) δ 8.62 (d, J=6.6 Hz, 2H), 8.54 (s, 2H), 8.20 (s, 1H), 8.13-8.07 (m, 3H), 7.86 (d, J=9.0 Hz, 1H), 7.57-7.53 (m, 3H), 5.31-5.26 (m, 1H), 4.77-4.65 (m, 3H), 4.43-4.35 (m, 1H), 4.24-4.14 (m, 2H), 4.05-3.94 (m, 1H), 2.71-2.55 (m, 1H), 2.30-2.17 (m, 1H), 1.59 (d, J=7.0 Hz, 3H). LCMS (ESI) (m/z): 578 (M+H)⁺.

### Scheme 227: Synthesis of (2S,4R)—N—((R)-1-(4-carbamimidoylthiophen-2-yl)ethyl)-1-((dibenzo[b,d]furan-2-carbonyl)glycyl)-4-(o-tolyl)pyrrolidine-2-carboxamide (Compound 236)

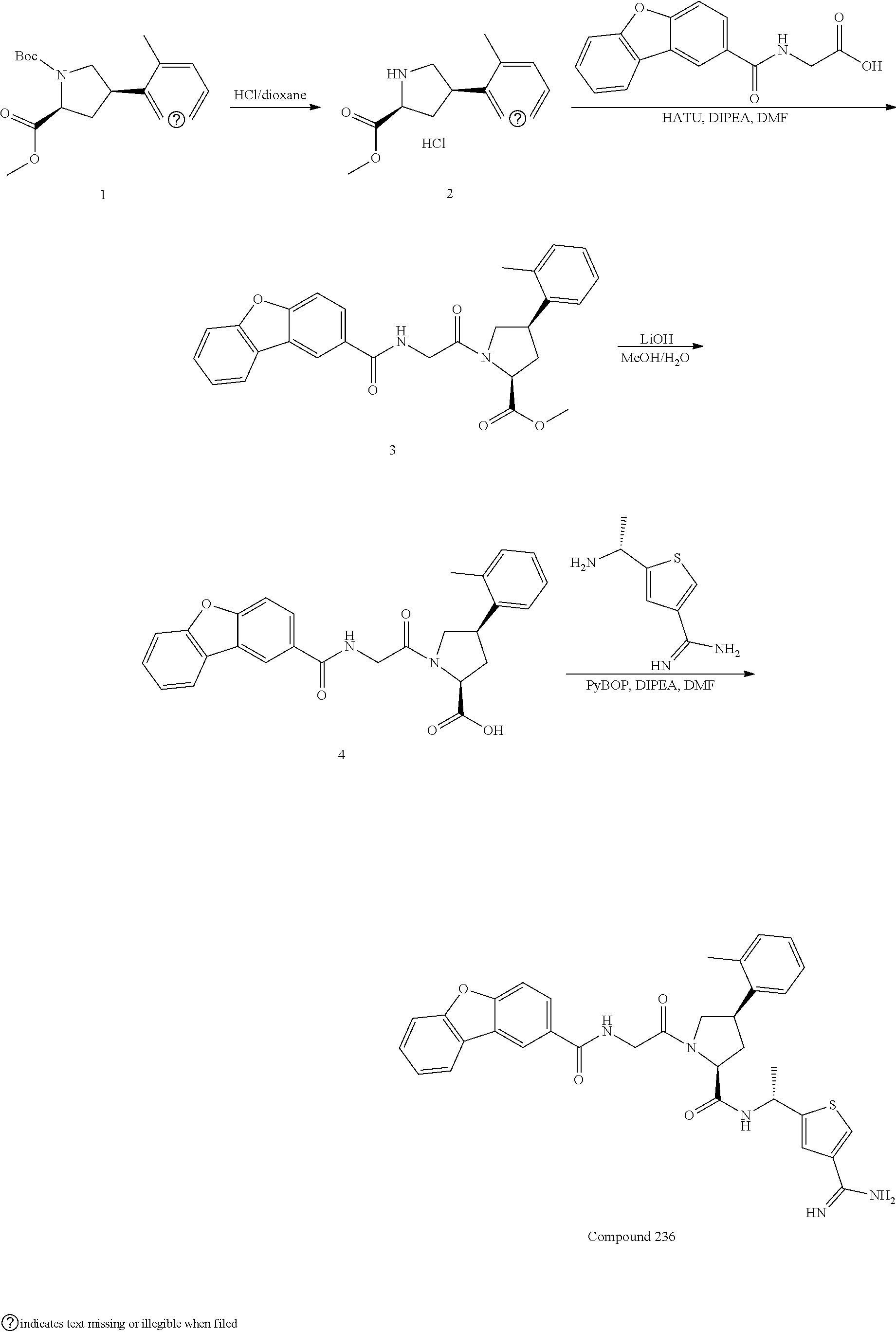

Compound 236 was prepared from 1-(tert-butyl) 2-methyl (S)-4-(o-tolyl)pyrrolidine-1,2-dicarboxylate, methyl (S)-4-(o-tolyl)pyrrolidine-2-carboxylate hydrochloride, and (R)-5-(1-aminoethyl)thiophene-3-carboximidamide based on the procedures set forth in Scheme 3. ¹H NMR (400 MHz, CD₃OD) δ 8.60 (s, 1H), 8.52 (s, 1H), 8.21 (s, 1H), 8.05 (dd, J=17.0, 8.2 Hz, 2H), 7.66 (dd, J=12.4, 8.4 Hz, 2H), 7.58-7.53 (m, 2H), 7.43 (t, J=7.5 Hz, 1H), 7.36 (d, J=7.2 Hz, 1H), 7.19 (dd, J=12.7, 5.9 Hz, 3H), 5.32 (d, J=6.9 Hz, 1H), 4.58 (s, 1H), 4.55-4.41 (m, 1H), 4.21 (dd, J=23.6, 12.3 Hz, 2H), 3.80 (d, J=6.5 Hz, 1H), 3.72-3.66 (m, 1H), 2.65 (dd, J=12.4, 6.3 Hz, 1H), 2.42 (d, J=16.8 Hz, 3H), 2.10 (dd, J=22.6, 12.0 Hz, 1H), 1.62 (t, J=13.3 Hz, 3H). LCMS (ESI) (m/z): 608 (M+H)⁺.

### Scheme 228: Synthesis of (S)—N—((R)-1-(4-carbamimidoylthiophen-2-yl)ethyl)-7-((9,9-difluoro-2-methyl-9H-fluorene-3-carbonyl)glycyl)-1,4-dioxa-7-azaspiro[4.4]nonane-8-carboxamide (Compound 237)

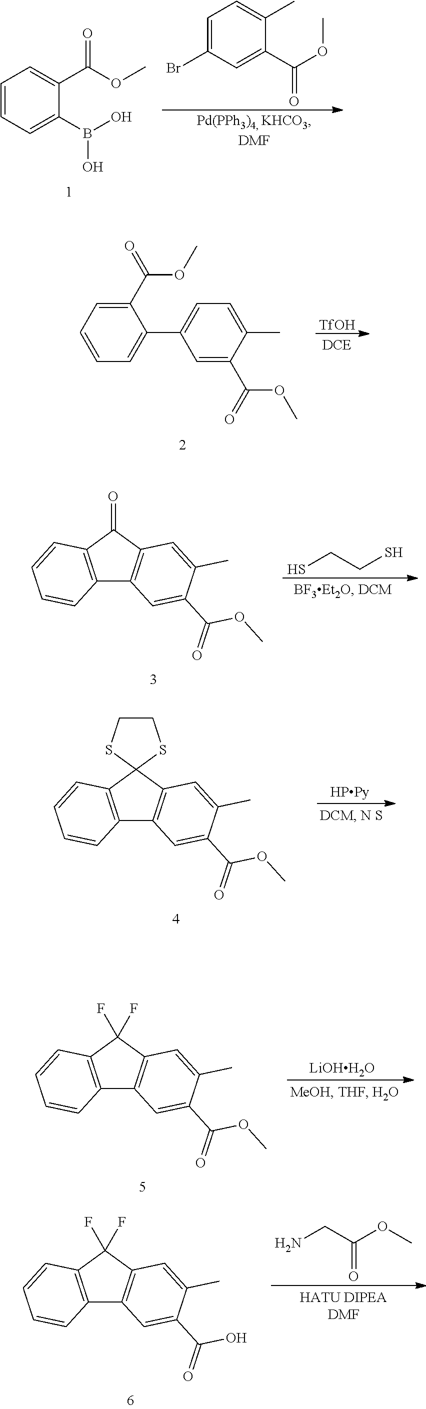

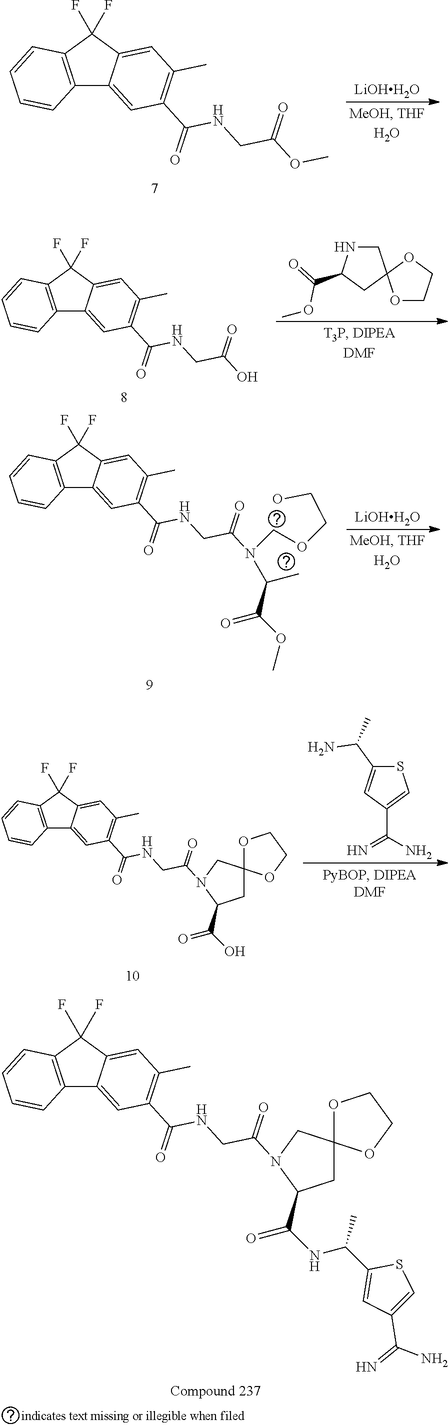

Compound 237 was prepared from methyl 5-bromo-2-methylbenzoate, (2-(methoxycarbonyl)phenyl)boronic acid, methyl glycinate, methyl (S)-1,4-dioxa-7-azaspiro[4.4]nonane-8-carboxylate, and (S)-5-(1-aminoethyl) thiophene-3-carboximidamide based on the procedures set forth in Scheme 170. ¹H NMR (400 MHz, CD₃OD) δ 8.48 (s, 1H), 8.23 (s, 1H), 7.79 (s, 1H), 7.68 (d, J=7.8 Hz, 1H), 7.60 (s, 1H), 7.51 (s, 2H), 7.41 (d, J=7.8 Hz, 1H), 5.30-5.29 (m, 1H), 4.75-4.74 (m, 1H), 4.19 (s, 2H), 3.99 (dd, J=10.2, 5.6 Hz, 4H), 3.79 (d, J=5.8 Hz, 2H), 2.47 (s, 3H), 2.25-2.20 (m, 1H), 1.58 (d, J=6.8 Hz, 3H). LCMS (ESI) (m/z): 624 (M+H)⁺.

### Scheme 229: Synthesis of (2S,4R)—N—((R)-1-(4-carbamimidoylthiophen-2-yl)ethyl)-1-((9,9-difluoro-9H-fluorene-3-carbonyl)glycyl)-4-(ethylsulfonyl)pyrrolidine-2-carboxamide (Compound 238)

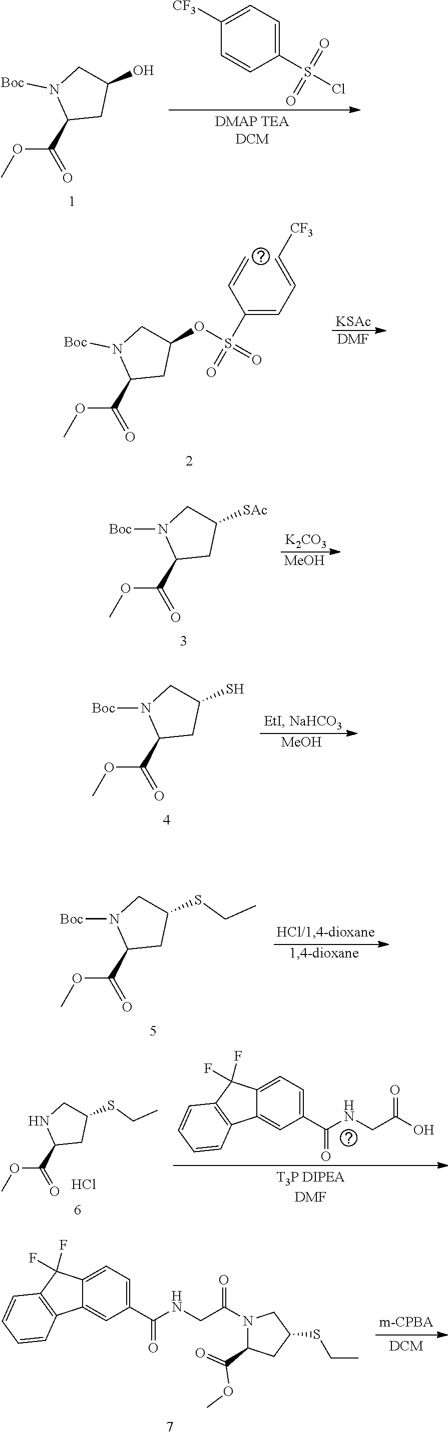

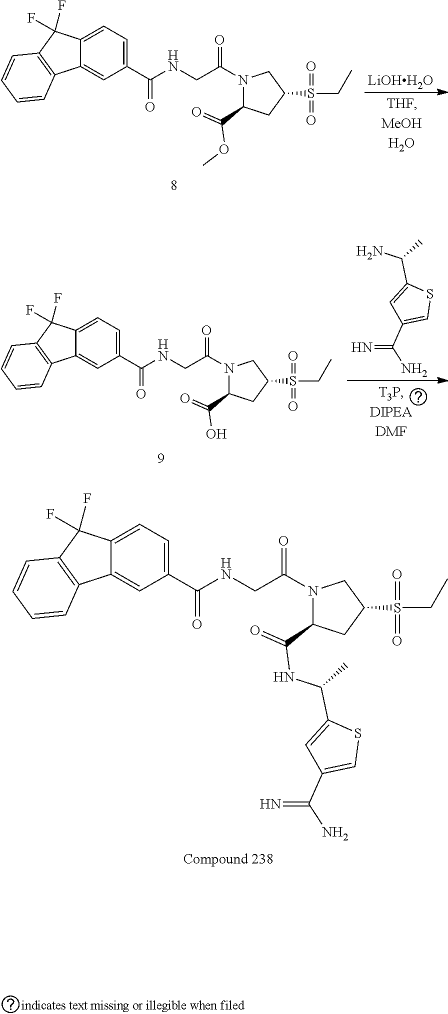

### Step 1: 1-(tert-butyl) 2-methyl (2S,4S)-4-(((4-(trifluoromethyl)phenyl)sulfonyl)oxy) pyrrolidine-1,2-dicarboxylate (2)

To a mixture of 1-(tert-butyl) 2-methyl (2S,4S)-4-hydroxypyrrolidine-1,2-dicarboxylate (10 g, 40.77 mmol) and 4-(trifluoromethyl)benzenesulfonyl chloride (13.0 g, 53.0 mmol) in DCM (50 mL) was added TEA (5.0 g, 48.9 mmol) and DMAP (498 mg, 4.08 mmol) at 0° C. under N₂ atmosphere and the reaction mixture was stirred at room temperature overnight. The mixture was diluted with EtOAc, washed with 1 N aq·HCl solution and brine, dried over anhydrous Na₂SO₄, filtered, and concentrated under reduced pressure to dryness. The residue was purified by flash chromatography (silica gel, 0-5% MeOH in DCM) to give 1-(tert-butyl) 2-methyl (2S,4S)-4-(((4-(trifluoromethyl)phenyl)sulfonyl)oxy)pyrrolidine-1,2-dicarboxylate (18.12 g, yield 98.0%) as a white solid. LC/MS (ESI) m/z: 454 (M+H)⁺.

### Step 2: 1-(tert-butyl) 2-methyl (2S,4R)-4-(acetylthio)pyrrolidine-1,2-dicarboxylate (3)

To a solution of 1-(tert-butyl) 2-methyl (2S,4S)-4-(((4-(trifluoromethyl)phenyl)sulfonyl)oxy) pyrrolidine-1,2-dicarboxylate (18.1 g, 40 mmol) in DMF (100 mL) was added KSAc (9.1 g, 79.9 mmol) and the reaction mixture was stirred under N₂ atmosphere at 40° C. overnight. The mixture was diluted with EtOAc and washed with water and brine, dried over anhydrous Na₂SO₄, filtered, and concentrated under reduced pressure to dryness. The residue was purified by flash chromatography (silica gel, 0-25% EtOAc in PE) to give 1-(tert-butyl) 2-methyl (2R,4S)-4-(acetylthio)pyrrolidine-1,2-dicarboxylate (11.05 g, yield 91.1%) as a white solid. LC/MS (ESI) m/z: 304 (M+H)⁺.

### Step 3: 1-(tert-butyl) 2-methyl (2S,4R)-4-mercaptopyrrolidine-1,2-dicarboxylate (4)

To a solution of 1-(tert-butyl) 2-methyl (2S,4R)-4-(acetylthio)pyrrolidine-1,2-dicarboxylate (1 g, 3.30 mmol) in MeOH/DCM (11 mL, 10/1) was added K₂CO₃ (456 mg, 3.30 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give 1-(tert-butyl) 2-methyl (2S,4R)-4-mercaptopyrrolidine-1,2-dicarboxylate (860 mg, yield 99.8%) as a white solid. LC/MS (ESI) m/z: 262 (M+H)⁺.

### Step 4: 1-(tert-butyl) 2-methyl (2S,4R)-4-(ethylthio)pyrrolidine-1,2-dicarboxylate (5)

To a solution of 1-(tert-butyl) 2-methyl (2S,4R)-4-mercaptopyrrolidine-1,2-dicarboxylate (660 mg, 2.53 mmol) in MeOH (10 mL) was added EtI (4.1 g, 26.01 mmol) and NaHCO₃ (233 mg, 2.78 mmol) under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-25% EtOAc in PE) to give 1-(tert-butyl) 2-methyl (2S,4R)-4-(ethylthio)pyrrolidine-1,2-dicarboxylate (570 mg, yield 78.0%) as a white solid. LC/MS (ESI) m/z: 290 (M+H)⁺.

### Step 5: methyl (2S,4R)-4-(ethylthio)pyrrolidine-2-carboxylate hydrochloride (6)

To a solution of 1-(tert-butyl) 2-methyl (2S,4R)-4-(ethylthio)pyrrolidine-1,2-dicarboxylate (80 mg, 0.28 mmol) in 1,4-dioxane (2 mL) was added HCl/1,4-dioxane (1 mL, 4 M) under N₂ atmosphere and the reaction mixture was stirred at room temperature overnight. The mixture was concentrated under reduced pressure to dryness to give methyl (2S,4R)-4-(ethylthio)pyrrolidine-2-carboxylate hydrochloride (65 mg, yield 100%) as a white solid, which was directly used in the next reaction without purification. LC/MS (ESI) m/z: 190 (M+H)⁺.

### Step 6: methyl (2S,4R)-1-((9,9-difluoro-9H-fluorene-3-carbonyl)glycyl)-4-(ethylthio) pyrrolidine-2-carboxylate (7)

To a mixture of (9,9-difluoro-9H-fluorene-3-carbonyl)glycine (80 mg, 0.26 mmol) and methyl (2S,4R)-4-(ethylthio)pyrrolidine-2-carboxylate hydrochloride (59 mg, 0.26 mmol) in DMF (3 mL) was added DIPEA (205 mg, 1.58 mmol) and T₃P (252 mg, 0.40 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-20% EtOAc in PE) to give methyl (2S,4R)-1-((9,9-difluoro-9H-fluorene-3-carbonyl)glycyl)-4-(ethylthio)pyrrolidine-2-carboxylate (50 mg, yield 39.9%) as a white solid. LC/MS (ESI) m/z: 475 (M+H)⁺.

### Step 7: methyl (2S,4R)-1-((9,9-difluoro-9H-fluorene-3-carbonyl)glycyl)-4-(ethylsulfonyl) pyrrolidine-2-carboxylate (8)

To a solution of methyl (2S,4R)-1-((9,9-difluoro-9H-fluorene-3-carbonyl)glycyl)-4-(ethylthio) pyrrolidine-2-carboxylate (50 mg, 0.11 mmol) in DCM (3 mL) was added m-CPBA (36 mg, 0.21 mmol) under N₂ atmosphere and the reaction mixture was stirred at room temperature for 3 hours. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give methyl (2S,4R)-1-((9,9-difluoro-9H-fluorene-3-carbonyl)glycyl)-4-(ethylsulfonyl)pyrrolidine-2-carboxylate (40 mg, yield 74.9%) as a white solid. LC/MS (ESI) m/z: 507 (M+H)⁺.

### Step 6: (2S,4R)-1-((9,9-difluoro-9H-fluorene-3-carbonyl)glycyl)-4-(ethylsulfonyl)pyrrolidine-2-carboxylic acid (9)

To a solution of methyl (2S,4R)-1-((9,9-difluoro-9H-fluorene-3-carbonyl)glycyl)-4-(ethylsulfonyl)pyrrolidine-2-carboxylate (40 mg, 0.079 mmol) in THF/MeOH/H₂O (3 mL, 4/1/1) was added LiOH·H₂O (7 mg, 0.16 mmol) and the reaction mixture was stirred at room temperature for 2 hours. The mixture was acidified with 1 N aq·HCl to pH ˜3 and extracted with DCM twice. The combined organic layers were washed with brine, dried over anhydrous Na₂SO₄, filtered, and concentrated under reduced pressure to dryness to give (2S,4R)-1-((9,9-difluoro-9H-fluorene-3-carbonyl)glycyl)-4-(ethylsulfonyl)pyrrolidine-2-carboxylic acid (35 mg, yield 90.0%) as a white solid. LC/MS (ESI) m/z: 493 (M+H)⁺.

### Step 9: (2S,4R)—N—((R)-1-(4-carbamimidoylthiophen-2-yl)ethyl)-1-((9,9-difluoro-9H-fluorene-3-carbonyl)glycyl)-4-(ethylsulfonyl)pyrrolidine-2-carboxamide (Compound 238)

To a mixture of (2S,4R)-1-((9,9-difluoro-9H-fluorene-3-carbonyl)glycyl)-4-(ethylsulfonyl) pyrrolidine-2-carboxylic acid (35 mg, 0.071 mmol) and (R)-5-(1-aminoethyl)thiophene-3-carboximidamide (24 mg, 0.14 mmol) in DMF (3 mL) was added DIPEA (55 mg, 0.43 mmol) and T₃P (68 mg, 0.11 mmol, 50% wt. in EtOAc) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=5:1) and further purified by prep-HPLC to give Compound 238 (2 mg, yield 4.4%) as a white solid. ¹H NMR (400 MHz, CD₃OD) δ 8.53 (s, 1H), 8.29-8.14 (m, 2H), 7.87 (t, J=9.5 Hz, 1H), 7.81-7.70 (m, 2H), 7.66 (d, J=7.0 Hz, 1H), 7.61-7.43 (m, 3H), 5.39-5.15 (m, 2H), 4.72 (d, J=4.0 Hz, 1H), 4.22-4.09 (m, 3H), 3.26-3.15 (m, 2H), 2.96-2.74 (m, 1H), 2.57-2.39 (m, 1H), 2.20-2.02 (m, 1H), 1.62 (t, J=15.4 Hz, 3H), 1.40 (t, J=7.5 Hz, 3H). LC/MS (ESI) m/z: 644 (M+H)⁺.

### Scheme 230: Synthesis of (2S,4R)-1-((anthracene-2-carbonyl)glycyl)-N—((R)-1-(4-carbamimidoylthiophen-2-yl)ethyl)-4-(methylsulfonyl)pyrrolidine-2-carboxamide (Compound 239)

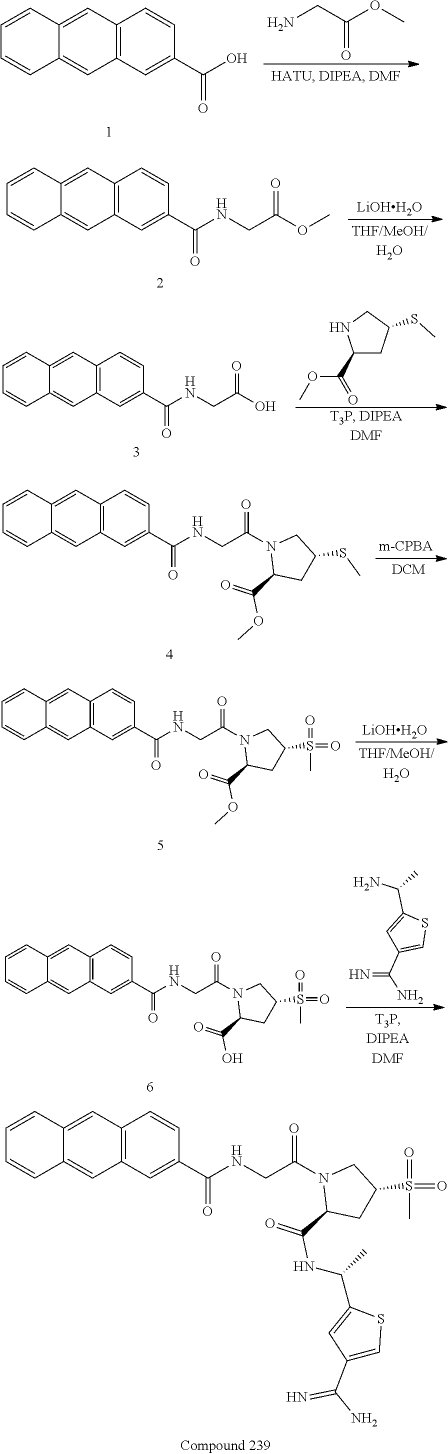

### Step 1: methyl (anthracene-2-carbonyl)glycinate (2)

To a mixture of anthracene-2-carboxylic acid (1.2 g, 5.40 mmol) and methyl glycinate (722 mg, 8.10 mmol) in DMF (30 mL) was added HATU (3.1 g, 8.10 mmol) and DIPEA (4.2 g, 32.40 mmol) under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-10% EtOAc in PE) to give methyl (anthracene-2-carbonyl)glycinate (1.23 g, yield 77.7%) as a white solid. LC/MS (ESI) m/z: 294 (M+H)⁺.

### Step 2: (anthracene-2-carbonyl)glycine (3)

To a solution of methyl (anthracene-2-carbonyl)glycinate (1.23 g, 4.19 mmol) in THF/MeOH/H₂O (18 mL, 4/1/1) was added LiOH·H₂O (352 mg, 8.39 mmol) and the reaction mixture was stirred at room temperature overnight. The mixture was acidified with 1 N aq·HCl to pH ˜4 and extracted with EtOAc three times. The combined organic layers were washed with brine, dried over anhydrous Na₂SO₄, filtered, and concentrated under reduced pressure to dryness to give (anthracene-2-carbonyl)glycine (1.16 g, yield 99.0%) as a white solid, which was directly used in the next reaction without purification. LC/MS (ESI) m/z: 280 (M+H)⁺.

### Step 3: methyl (2S,4R)-1-((anthracene-2-carbonyl)glycyl)-4-(methylthio)pyrrolidine-2-carboxylate (4)

To a mixture of (anthracene-2-carbonyl)glycine (80 mg, 0.29 mmol) and methyl (2S,4R)-4-(methylthio)pyrrolidine-2-carboxylate (50 mg, 0.29 mmol) in DMF (3 mL) was added DIPEA (222 mg, 1.72 mmol) and T₃P (273 mg, 0.43 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-25% EtOAc in PE) to give methyl (2S,4R)-1-((anthracene-2-carbonyl)glycyl)-4-(methylthio)pyrrolidine-2-carboxylate (80 mg, yield 64.0%) as a white solid. LC/MS (ESI) m/z: 437 (M+H)⁺.

### Step 4: methyl (2S,4R)-1-((anthracene-2-carbonyl)glycyl)-4-(methylsulfonyl)pyrrolidine-2-carboxylate (5)

To a solution of methyl (2S,4R)-1-((anthracene-2-carbonyl)glycyl)-4-(methylthio)pyrrolidine-2-carboxylate (80 mg, 0.18 mmol) in DCM (3 mL) was added m-CPBA (63 mg, 0.37 mmol) under N₂ atmosphere and the reaction mixture was stirred at room temperature for 3 hours. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give methyl (2S,4R)-1-((anthracene-2-carbonyl)glycyl)-4-(methylsulfonyl)pyrrolidine-2-carboxylate (60 mg, yield 69.9%) as a white solid. LC/MS (ESI) m/z: 469 (M+H)⁺.

### Step 5: (2S,4R)-1-((anthracene-2-carbonyl)glycyl)-4-(methylsulfonyl)pyrrolidine-2-carboxylic acid (6)

To a solution of methyl (2S,4R)-1-((anthracene-2-carbonyl)glycyl)-4-(methylsulfonyl) pyrrolidine-2-carboxylate (60 mg, 0.13 mmol) in THF/MeOH/H₂O (6 mL, 4/1/1) was added LiOH·H₂O (11 mg, 0.26 mmol) and the reaction mixture was stirred at room temperature overnight. The mixture was acidified with 1 N aq·HCl to pH˜4 and extracted with EtOAc three times. The combined organic layers were washed with brine, dried over anhydrous Na₂SO₄, filtered, and concentrated under reduced pressure to dryness to give (2S,4R)-1-((anthracene-2-carbonyl)glycyl)-4-(methylsulfonyl)pyrrolidine-2-carboxylic acid (55 mg, yield 94.5%) as a white solid. LC/MS (ESI) m/z:455 (M+H)⁺.

### Step 6: (2S,4R)-1-((anthracene-2-carbonyl)glycyl)-N—((R)-1-(4-carbamimidoylthiophen-2-yl) ethyl)-4-(methylsulfonyl)pyrrolidine-2-carboxamide (Compound 239)

To a mixture of (2S,4R)-1-((anthracene-2-carbonyl)glycyl)-4-(methylsulfonyl) pyrrolidine-2-carboxylic acid (55 mg, 0.12 mmol) and (R)-5-(1-aminoethyl)thiophene-3-carboximidamide (25 mg, 0.15 mmol) in DMF (3 mL) was added DIPEA (94 mg, 0.73 mmol) and T₃P (116 mg, 0.18 mmol, 50% wt. in EtOAc) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=5:1) and further purified by prep-HPLC to give Compound 239 (1.5 mg, yield 2.0%) as a white solid. ¹H NMR (400 MHz, CD₃OD) δ 8.63-8.53 (m, 3H), 8.21 (t, J=9.2 Hz, 1H), 8.14-8.05 (m, 3H), 7.85 (dt, J=9.2, 4.6 Hz, 1H), 7.59-7.50 (m, 3H), 5.31 (dt, J=14.0, 6.2 Hz, 2H), 4.25-4.15 (m, 3H), 3.08 (d, J=19.4 Hz, 3H), 2.79 (dd, J=13.3, 7.2 Hz, 1H), 2.58-2.43 (m, 1H), 2.19 (t, J=7.4 Hz, 1H), 2.03 (d, J=6.3 Hz, 1H), 1.64 (d, J=25.8 Hz, 3H). LC/MS (ESI) m/z: 606 (M+H)⁺.

### Scheme 231: (S)—N-((4-carbamimidoylthiophen-2-yl)methyl)-7-(2-(5-(2,4-dimethylphenyl)-1-oxoisoindolin-2-yl)acetyl)-1,4-dioxa-7-azaspiro[4.4]nonane-8-carboxamide (Compound 240)

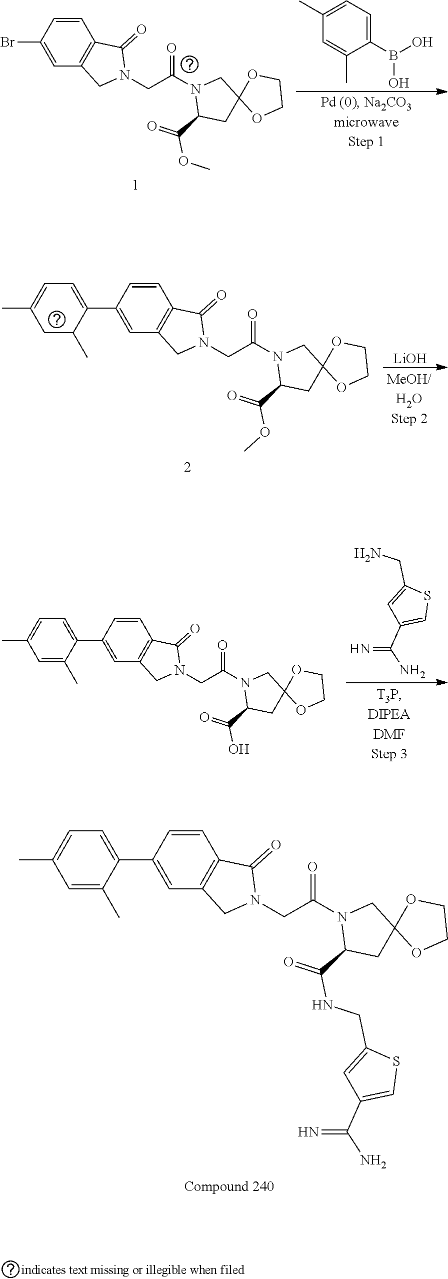

Compound 240 was prepared from (2,4-dimethylphenyl)boronic acidmethyl (8S)-7-[2-(5-bromo-1-oxo-isoindolin-2-yl)acetyl]-1,4-dioxa-7-azaspiro[4.4]nonane-8-carboxylate, and 5-(aminomethyl)thiophene-3-carboximidamide. HCl based on the procedures set forth in Scheme 199. LC/MS (ESI) m/z: 588 (M+H)⁺. ¹H NMR (400 MHz, DMSO) δ 8.66 (s, 1H), 8.34 (s, 1H), 7.68 (d, J=7.8 Hz, 1H), 7.47 (s, 1H), 7.35 (d, J=7.8 Hz, 1H), 7.04 (dd, J=16.6, 7.5 Hz, 2H), 4.54-4.23 (m, 3H), 3.95-3.77 (m, 4H), 3.74 (d, J=10.8 Hz, 3H), 3.67-3.02 (m, 4H), 2.92 (p, J=6.6 Hz, 2H), 2.43 (d, J=3.0 Hz, 3H), 2.25 (s, 3H), 2.14-2.10 (m, 1H), 2.07-1.89 (m, 1H)

### Scheme 232: (S)—N-((4-carbamimidoylthiophen-2-yl)methyl)-7-((2′-fluoro-6′-methoxy-[1,1′-biphenyl]-4-carbonyl)glycyl)-1,4-dioxa-7-azaspiro[4.4]nonane-8-carboxamide (Compound 241)

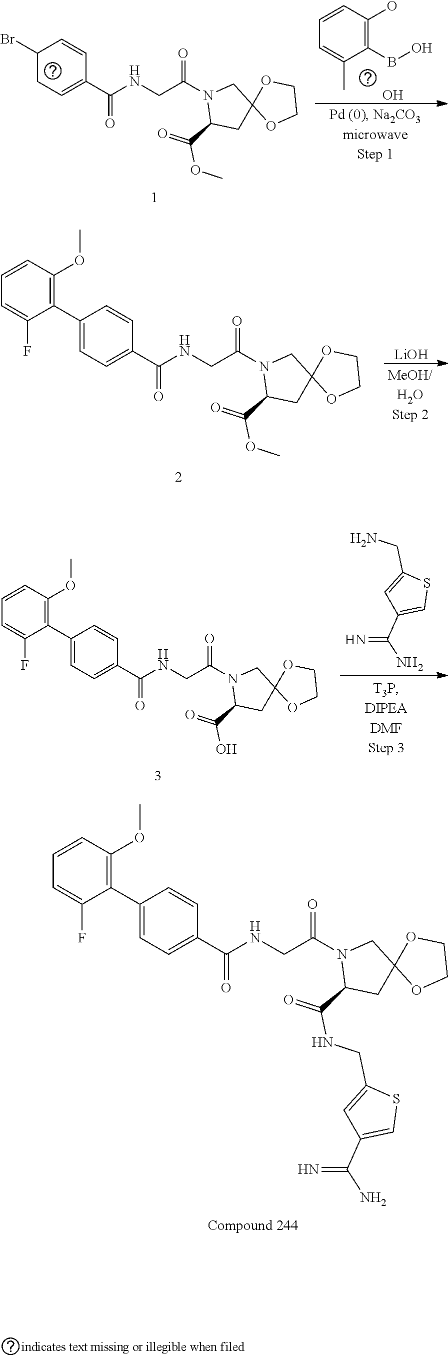

Compound 244 was prepared from (2-fluoro-6-methoxy-phenyl)boronic acid, methyl (8S)-7-[2-[(4-bromobenzoyl)amino]acetyl]-1,4-dioxa-7-azaspiro[4.4]nonane-8-carboxylate, and 5-(aminomethyl)thiophene-3-carboximidamide. HCl based on the procedures set forth in Scheme 199. LC/MS (ESI) m/z: 596 (M+H)⁺. ¹H NMR (400 MHz, DMSO) δ 8.62 (d, J=16.2 Hz, 1H), 8.33 (s, 1H), 7.84 (d, J=7.8 Hz, 1H), 7.81-7.56 (m, 2H), 7.35 (dd, J=12.0, 7.7 Hz, 2H), 7.06 (s, 1H), 6.92 (d, J=8.4 Hz, 1H), 6.85 (t, J=8.9 Hz, 1H), 4.58-4.27 (m, 2H), 4.03-3.77 (m, 4H), 3.68-3.54 (m, 3H), 3.27 (s, 3H), 2.92 (p, J=6.5 Hz, 2H), 2.57-2.29 (m, 1H), 2.25 (dd, J=12.9, 9.1 Hz, 1H).

### Scheme 233: (S)-4′-((2-(8-(((4-carbamimidoylthiophen-2-yl)methyl)carbamoyl)-1,4-dioxa-7-azaspiro[4.4]nonan-7-yl)-2-oxoethyl)carbamoyl)-2-fluoro-[1,1′-biphenyl]-4-carboxylic acid (Compound 242)

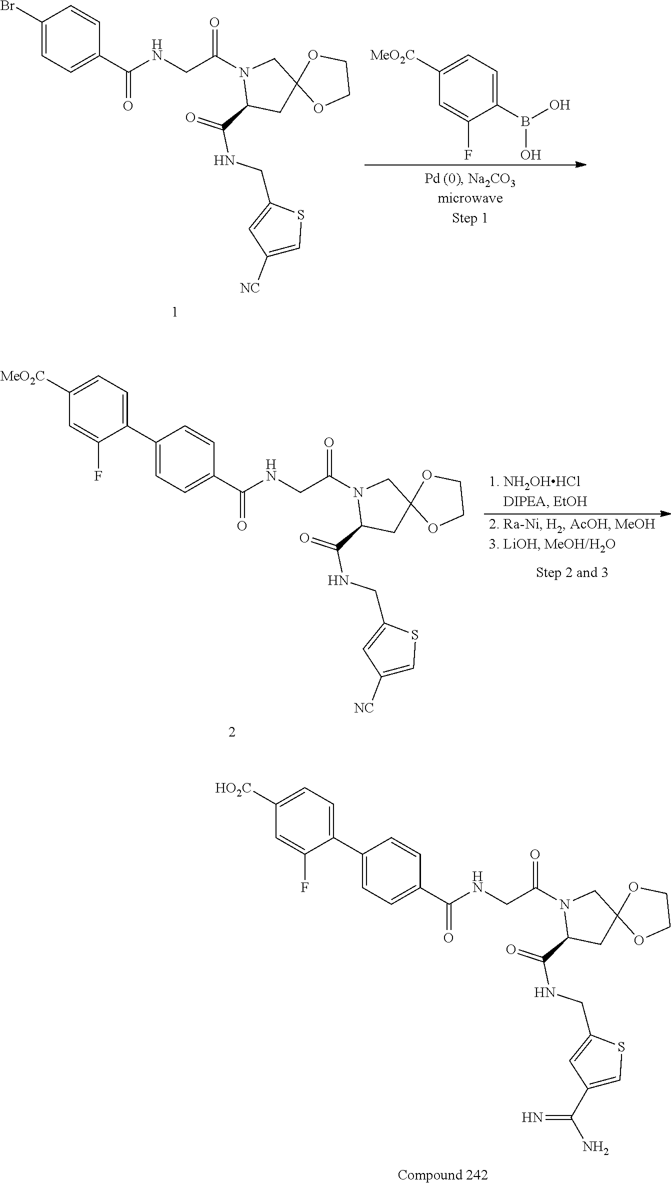

Compound 242 was prepared from (4-acetoxy-2-fluoro-phenyl)boronic acid, (8S)-7-[2-[(4-bromobenzoyl)amino]acetyl]-N-[(5-cyano-3-thienyl)methyl]-1,4-dioxa-7-azaspiro[4.4]nonane-8-carboxamide, and NH₂OH·HCl based on the procedures set forth in Scheme 218. LC/MS (ESI) m/z: 610 (M+H)⁺. ¹H NMR (400 MHz, DMSO) δ 11.89 (s, 1H), 8.92 (s, 1H), 8.77-8.66 (m, 1H), 8.61 (s, 1H), 8.51 (t, J=6.1 Hz, 1H), 8.28 (d, J=9.3 Hz, 1H), 7.88 (dd, J=12.1, 8.0 Hz, 1H), 7.73-7.40 (m, 3H), 7.29 (q, J=7.7 Hz, 1H), 4.59-4.25 (m, 4H), 4.23-3.95 (m, 3H), 3.78-3.57 (m, 4H), 2.30 (dd, J=13.2, 8.9 Hz, 1H), 2.01 (dd, J=13.4, 6.6 Hz, 1H).

### Scheme 234: (S)—N-((4-aminothiophen-2-yl)methyl)-7-((4-(4-fluorophenoxy)benzoyl)glycyl)-1,4-dioxa-7-azaspiro[4.4]nonane-8-carboxamide (Compound 244)

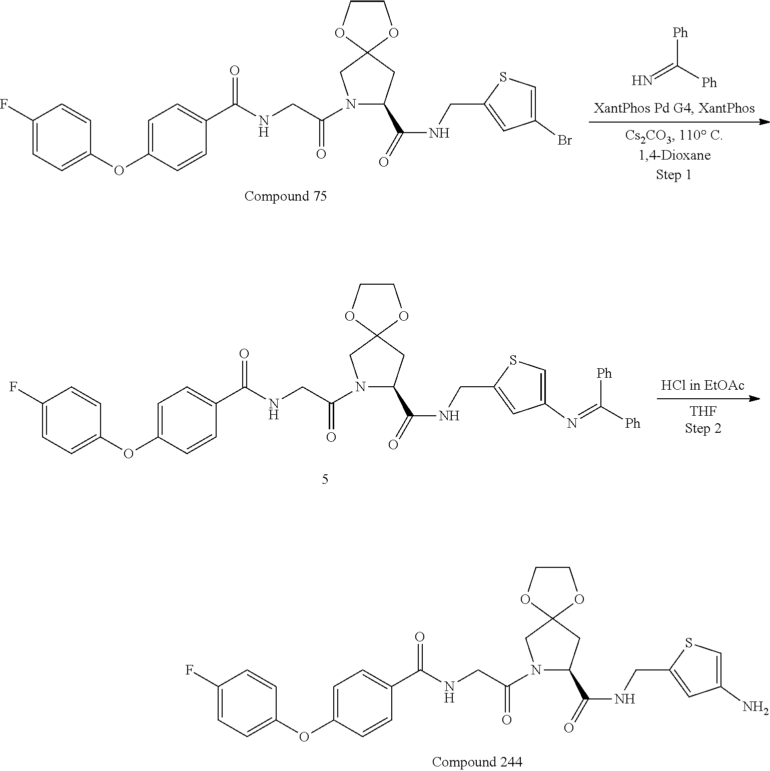 [SMILES: O=C(NCC(=O)N1CC2(C[C@H]1C(=O)NCc1cc(Br)cs1)OCCO2)c1ccc(Oc2ccc(F)cc2)cc1 ; N=C(c1ccccc1)c1ccccc1 ; O=C(NCC(=O)N1CC2(C[C@H]1C(=O)NCc1cc(N=C(c3ccccc3)c3ccccc3)cs1)OCCO2)c1ccc(Oc2ccc(F)cc2)cc1 ; Nc1csc(CNC(=O)[C@@H]2CC3(CN2C(=O)CNC(=O)c2ccc(Oc4ccc(F)cc4)cc2)OCCO3)c1]

### Step 1: (S)—N-((4-((diphenylmethylene)amino)thiophen-2-yl)methyl)-7-((4-(4-fluorophenoxy)benzoyl)glycyl)-1,4-dioxa-7-azaspiro[4.4]nonane-8-carboxamide (5)

A solution of Compound 75 (0.500 g, 0.808 mmol, 1.0 equiv.) in 1,4-Dioxane (4.00 mL) (5.0 mL) was purged with argon, Benzophenone imine (1.0 mL, 6.0 mmol, 7.4 equiv.) was added and purged, Cesium carbonate (0.790 g, 2.42 mmol, 3.0 equiv.), XantPhos Pd G4 (0.198 g, 0.202 mmol, 2.5 equiv.) and XantPhos (0.117 g, 0.202 mmol, 2.5 equiv.) was added and purged. The mixture was stirred at 110° C. for 2 hours. LCMS showed the presence of the desired product. The mixture was passed through a pad of celite, and the filtrate was concentrated. The residue was purified by column chromatography (24 g Gold, SiO₂) using 0-10% MeOH in DCM to obtain the desired product (S)—N-((4-((diphenylmethylene)amino)thiophen-2-yl)methyl)-7-((4-(4-fluorophenoxy)benzoyl)glycyl)-1,4-dioxa-7-azaspiro[4.4]nonane-8-carboxamide, 5 (0.2 g, 0.278 mmol, 34% yield). LC/MS (ESI) m/z: 719 (M+H)⁺.

### Step 2: (S)—N-((4-aminothiophen-2-yl)methyl)-7-((4-(4-fluorophenoxy)benzoyl)glycyl)-1,4-dioxa-7-azaspiro[4.4]nonane-6-carboxamide (Compound 244)

To a solution of 5 (100 mg, 0.1391 mmol, 1.0 equiv.) in THF (5.0 mL) was added Hydrochloric acid (1.0 mol/L) in EtOAc (2.0 mL, 2.0 mmol, 1 mol/L, 14.0 equiv.)). The solution was stirred for 5 h. LC-MS indicated the completion of the reaction. Solvent was removed and the residue was purified by column chromatography (12 g gold, SiO₂) using 0-10% MeOH in DCM to obtain desired product Compound 244 (9.0 mg, 0.016 mmol, 11.7% yield) as a beige solid. LC/MS (ESI) m/z: 555 (M+H)⁺. ¹H NMR (400 MHz, DMSO) δ 8.56 (d, J=6.4 Hz, 2H), 8.35 (t, J=6.0 Hz, 1H), 7.89 (dd, J=8.8, 3.3 Hz, 3H), 7.27 (t, J=8.6 Hz, 3H), 7.19-7.11 (m, 3H), 7.02 (d, J=8.5 Hz, 3H), 6.44 (s, 1H), 5.81-5.73 (m, 2H), 4.67 (s, 3H), 4.41-4.22 (m, 3H), 4.22-4.05 (m, 1H), 3.93 (ddd, J=14.8, 9.8, 5.7 Hz, 7H), 3.78 (d, J=10.9 Hz, 1H), 3.68 (t, J=11.7 Hz, 1H), 3.59 (d, J=11.3 Hz, 1H), 2.28 (dd, J=13.2, 8.7 Hz, 1H), 2.02 (dd, J=13.2, 7.2 Hz, 1H), 1.76 (d, J=6.4 Hz, 1H).

### Scheme 235. Synthesis of (S)—N-((4-carbamimidoylthiophen-2-yl)methyl)-7-(2-(6-(2,4-difluorophenyl)-1-oxo-3,4-dihydroisoquinolin-2(1H)-yl)acetyl)-1,4-dioxa-7-azaspiro[4.4]nonane-8-carboxamide (Compound 245)

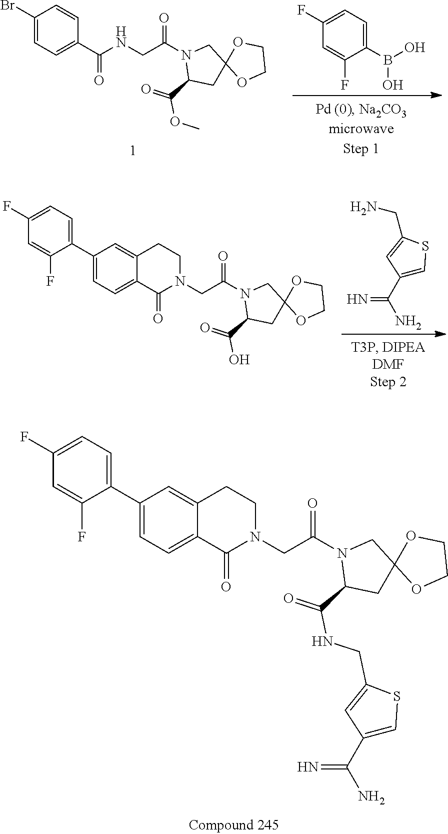 [SMILES: COC(=O)[C@@H]1CC2(CN1C(=O)CNC(=O)c1ccc(Br)cc1)OCCO2 ; OB(O)c1ccc(F)cc1F ; O=C(O)[C@@H]1CC2(CN1C(=O)CN1CCc3cc(-c4ccc(F)cc4F)ccc3C1=O)OCCO2 ; N=C(N)c1csc(CN)c1 ; N=C(N)c1csc(CNC(=O)[C@@H]2CC3(CN2C(=O)CN2CCc4cc(-c5ccc(F)cc5F)ccc4C2=O)OCCO3)c1]

Compound 245 was prepared from (2,4-difluorophenyl)boronic acid, methyl (8S)-7-[2-(6-bromo-1-oxo-3,4-dihydroisoquinolin-2-yl)acetyl]-1,4-dioxa-7-azaspiro[4.4]nonane-8-carboxylate, and 5-(aminomethyl)thiophene-3-carboximidamide. HCl based on the procedures set forth in Scheme 206. LC/MS (ESI) m/z: 610 (M+H)⁺. ¹H NMR (400 MHz, DMSO) δ 9.05 (s, 1H), 8.66 (s, 1H), 8.40 (s, 1H), 7.93 (d, J=7.8 Hz, 1H), 7.65 (s, 1H), 7.51 (d, J=9.9 Hz, 2H), 7.39 (t, J=10.1 Hz, 1H), 7.29-7.00 (m, 2H), 4.77-4.31 (m, 4H), 4.21 (d, J=16.2 Hz, 2H), 4.05-3.82 (m, 3H), 3.83-3.66 (m, 3H), 3.72-2.73 (m, 2H), 2.50 (p, J=1.8 Hz, 2H), 2.33-2.22 (m, 1H), 2.06-1.97 (m, 1H).

### Scheme 236: Synthesis of (S)—N—((R*)-1-(4-carbamimidoylthiophen-2-yl)-2,2,2-trifluoroethyl)-7-((9,9-difluoro-9H-fluorene-3-carbonyl)glycyl)-1,4-dioxa-7-azaspiro[4.4]nonane-8-carboxamide (Compound 246)

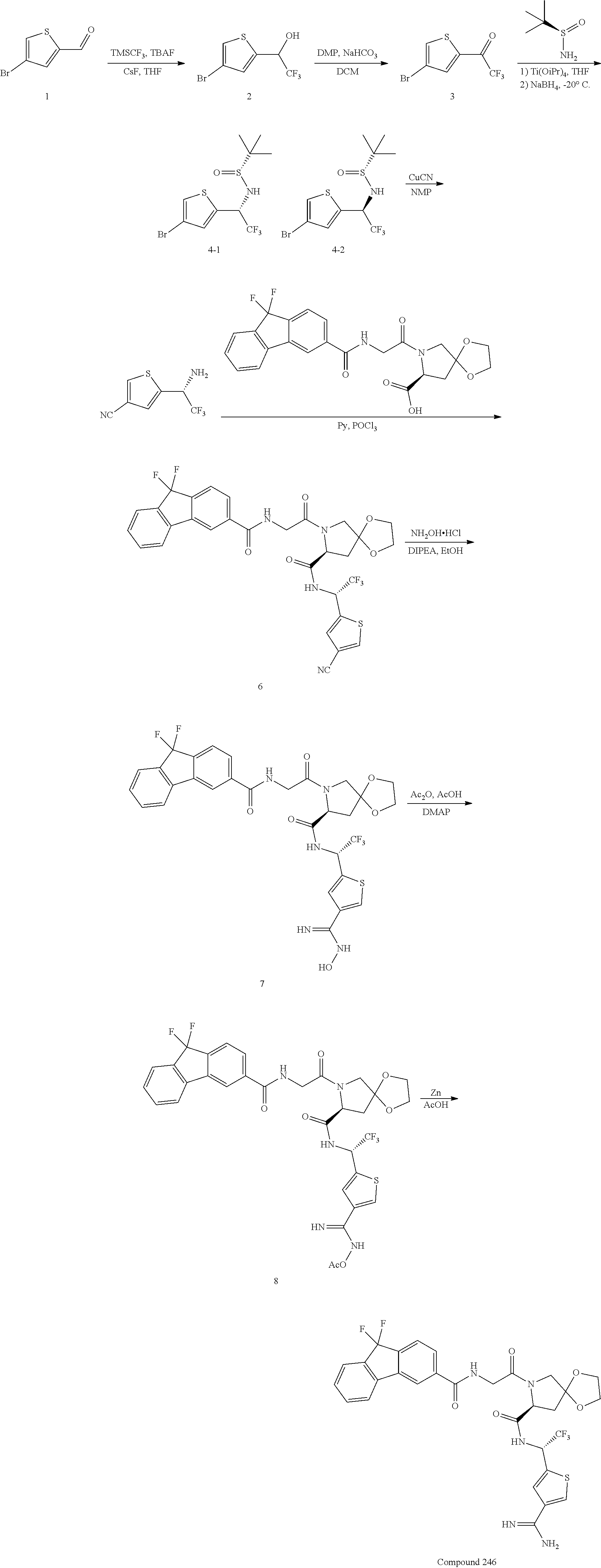

### Step 1: 1-(4-bromothiophen-2-yl)-2,2,2-trifluoroethan-1-ol (2)

To a mixture of 4-bromothiophene-2-carbaldehyde (5 g, 26.17 mmol) and CsF (3.98 g, 26.17 mmol) in THF (50 mL) was added trimethyl(trifluoromethyl)silane (3.98 g, 26.17 mmol) at 0° C. dropwise and the mixture was stirred at room temperature for 6 hours. The reaction mixture was added TBAF (13 mL, 13.09 mmol, 1M, in THF) and the mixture was stirred overnight. The reaction mixture was quenched with saturated aqueous NH₄HCl and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give 1-(4-bromothiophen-2-yl)-2,2,2-trifluoroethan-1-ol (5.72 g, yield 84.1%) as a brown oil. H NMR (400 MHz, CDCl₃) δ 7.28 (d, J=1.1 Hz, 1H), 7.09 (s, 1H), 5.24-5.17 (m, 1H).

### Step 2: 1-(4-bromothiophen-2-yl)-2,2,2-trifluoroethan-1-one (3)

To a mixture of 1-(4-bromothiophen-2-yl)-2,2,2-trifluoroethan-1-ol (5.72 g, 0.022 mol) and NaHCO₃ (5.5 g, 0.066 mol) in DCM (60 mL) was added DMP (14 g, 0.033 mmol) at 0° C. in portions and the mixture was stirred at room temperature for 18 hours. The mixture was quenched with saturated aqueous Na₂S₂O₃, diluted with DCM, washed with water and brine, dried over anhydrous Na₂SO₄, filtered, and concentrated under reduced pressure to dryness. The residue was purified by flash chromatography (silica gel, 0-50% EtOAc in PE) to give 1-(4-bromothiophen-2-yl)-2,2,2-trifluoroethan-1-one (3.6 g, yield 64.3%) as a colorless oil.

### Step 3: (R)—N—((S*)-1-(4-bromothiophen-2-yl)-2,2,2-trifluoroethyl)-2-methylpropane-2-sulfinamide (4-1) and (R)—N—((R*)-1-(4-bromothiophen-2-yl)-2,2,2-trifluoroethyl)-2-methylpropane-2-sulfinamide (4-2)

To a mixture of 1-(4-bromothiophen-2-yl)-2,2,2-trifluoroethan-1-one (3.6 g, 0.014 mol) and (R)-2-methylpropane-2-sulfinamide (3.4 g, 0.028 mol) in THF (35 mL) was added Ti(OiPr)₄ (11.9 g, 0.042 mol). The mixture was stirred under N₂ atmosphere at 50° C. for 3 hours. The mixture was cooled down to −20° C., and the mixture was added NaBH₄ (11.9 g, 0.042 mol) in portions. The mixture was stirred at −20° C. for 3 hours before quenched with saturated aqueous NH₄Cl and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) and further purified by prep-HPLC to give (R)—N—((S*)-1-(4-bromothiophen-2-yl)-2,2,2-trifluoroethyl)-2-methylpropane-2-sulfinamide (1 g, yield 19.6%) as a colorless oil and (R)—N—((R*)-1-(4-bromothiophen-2-yl)-2,2,2-trifluoroethyl)-2-methylpropane-2-sulfinamide (2 g, yield 39.2%) as a colorless oil. LCMS (ESI) m/z: 364 (M+H)⁺.

### Step 4: (R*)-5-(1-amino-2,2,2-trifluoroethyl)thiophene-3-carbonitrile (5)

To a mixture of (R)—N—((R*)-1-(4-bromothiophen-2-yl)-2,2,2-trifluoroethyl)-2-methylpropane-2-sulfinamide (979 mg, 2.71 mmol) in NMP (10 mL) was added CuCN (972 mg, 10.85 mol) and the mixture was stirred at 180° C. for 1 hour. The reaction mixture was diluted with EtOAc, washed with water and brine, dried over anhydrous Na₂SO₄, filtered, and concentrated under reduced pressure to dryness. The residue was purified by flash chromatography (silica gel, 0-70% EtOAc in PE) to give (R*)-5-(1-amino-2,2,2-trifluoroethyl)thiophene-3-carbonitrile (190 mg, yield 33.9%) as a yellow oil. LCMS (ESI) m/z: 207 (M+H)⁺.

### Step 5: (S)—N—((R*)-1-(4-cyanothiophen-2-yl)-2,2,2-trifluoroethyl)-7-((9,9-difluoro-9H-fluorene-3-carbonyl)glycyl)-1,4-dioxa-7-azaspiro[4.4]nonane-6-carboxamide (6)

To a mixture of (S)-7-((9,9-difluoro-9H-fluorene-3-carbonyl)glycyl)-1,4-dioxa-7-azaspiro[4.4]nonane-8-carboxylic acid (260 mg, 0.57 mmol) and (R*)-5-(1-amino-2,2,2-trifluoroethyl)thiophene-3-carbonitrile (153 mg, 0.68 mol) in pyridine (3 mL) was added POCl₃ (0.1 mL, 1.14 mmol) at 0° C. dropwise and the mixture was stirred at room temperature for 2 hours. The reaction mixture was concentrated under reduced pressure to dryness. The residue was purified by flash chromatography (silica gel, 0-70% EtOAc in PE) to give (S)—N—((R*)-1-(4-cyanothiophen-2-yl)-2,2,2-trifluoroethyl)-7-((9,9-difluoro-9H-fluorene-3-carbonyl) glycyl)-1,4-dioxa-7-azaspiro[4.4]nonane-8-carboxamide (170 mg, yield 46.2%) as a yellow oil. LCMS (ESI) m/z: 647 (M+H)⁺.

### Step 6: (S)-7-((9,9-difluoro-9H-fluorene-3-carbonyl)glycyl)-N—((R*)-2,2,2-trifluoro-1-(4-(N-hydroxycarbamimidoyl)thiophen-2-yl)ethyl)-1,4-dioxa-7-azaspiro[4.4]nonane-8-carboxamide (7)

To a mixture of (S)—N—((R*)-1-(4-cyanothiophen-2-yl)-2,2,2-trifluoroethyl)-7-((9,9-difluoro-9H-fluorene-3-carbonyl)glycyl)-1,4-dioxa-7-azaspiro[4.4]nonane-8-carboxamide (170 mg, 0.26 mmol) and DIPEA (102 mg, 0.79 mmol) in EtOH (2 mL) was added hydroxylamine hydrochloride (55 mg, 0.79 mmol) and the mixture was stirred at room temperature for 3 hours. The mixture was diluted with EtOAc, washed with water and brine, dried over anhydrous Na₂SO₄, filtered, and concentrated under reduced pressure to dryness to give (S)-7-((9,9-difluoro-9H-fluorene-3-carbonyl)glycyl)-N—((R*)-2,2,2-trifluoro-1-(4-(N-hydroxycarbamimidoyl) thiophen-2-yl)ethyl)-1,4-dioxa-7-azaspiro[4.4]nonane-8-carboxamide (100 mg, crude) as a yellow oil, which was used directly in the next reaction without further purification. LCMS (ESI) m/z: 680 (M+H)⁺.

### Step 7: (S)—N—((R*)-1-(4-(N-acetoxycarbamimidoyl)thiophen-2-yl)-2,2,2-trifluoroethyl)-7-((9,9-difluoro-9H-fluorene-3-carbonyl)glycyl)-1,4-dioxa-7-azaspiro[4.4]nonane-8-carboxamide (8)

To a mixture of (S)-7-((9,9-difluoro-9H-fluorene-3-carbonyl)glycyl)-N—((R*)-2,2,2-trifluoro-1-(4-(N-hydroxycarbamimidoyl)thiophen-2-yl)ethyl)-1,4-dioxa-7-azaspiro[4.4]nonane-8-carboxamide (100 mg, 0.15 mmol) in Ac₂O (1 mL) and AcOH (1 mL) was added DMAP (2 mg, 0.015 mol) and the reaction mixture was stirred at room temperature for 18 hours. The reaction mixture was concentrated under reduced pressure to dryness. The residue was purified by flash chromatography (silica gel, 0-15% MeOH in DCM) to give (S)—N—((R*)-1-(4-(N-acetoxycarbamimidoyl)thiophen-2-yl)-2,2,2-trifluoroethyl)-7-((9,9-difluoro-9H-fluorene-3-carbonyl)glycyl)-1,4-dioxa-7-azaspiro[4.4]nonane-8-carboxamide (62 mg, yield 57.4%) as a yellow solid. LCMS (ESI) m/z: 722 (M+H)⁺.

### Step 8: (S)—N—((R*)-1-(4-carbamimidoylthiophen-2-yl)-2,2,2-trifluoroethyl)-7-((9,9-difluoro-9H-fluorene-3-carbonyl)glycyl)-1,4-dioxa-7-azaspiro[4.4]nonane-8-carboxamide (Compound 246)

To a mixture of (S)—N—((R*)-1-(4-(N-acetoxycarbamimidoyl)thiophen-2-yl)-2,2,2-trifluoroethyl)-7-((9,9-difluoro-9H-fluorene-3-carbonyl)glycyl)-1,4-dioxa-7-azaspiro[4.4]nonane-8-carboxamide (30 mg, 0.042 mmol) in AcOH (1 mL) was added Zn (3 mg, 0.042 mol) and the reaction mixture was stirred at 50° C. for 18 hours. The reaction mixture was concentrated under reduced pressure to dryness. The residue was purified by prep-HPLC to give Compound 246 (1.1 mg, yield 4.0%) as a white solid. ¹H NMR (400 MHz, CD₃OD) δ 8.56-8.51 (m, 1H), 8.29 (s, 1H), 8.16 (d, J=13.4 Hz, 1H), 7.85 (d, J=8.3 Hz, 1H), 7.75-7.70 (m, 2H), 7.68-7.63 (m, 2H), 7.59 (t, J=7.0 Hz, 1H), 7.46 (t, J=7.2 Hz, 1H), 6.21-6.07 (m, 1H), 4.70 (d, J=5.1 Hz, 1H), 4.27 (d, J=16.3 Hz, 1H), 4.13 (d, J=16.3 Hz, 1H), 4.04-3.96 (m, 4H), 3.90-3.80 (m, 2H), 2.49 (dd, J=13.1, 8.8 Hz, 1H), 2.21 (d, J=6.7 Hz, 1H). LCMS (ESI) m/z: 664 (M+H)⁺.

### Scheme 237: Synthesis of methyl ((E)-amino(5-((R)-1-((2S,4R)-1-((9,9-difluoro-9H-fluorene-3-carbonyl)glycyl)-4-(difluoromethoxy)pyrrolidine-2-carboxamido)ethyl)thiophen-3-yl)methylene)carbamate (Compound 247)

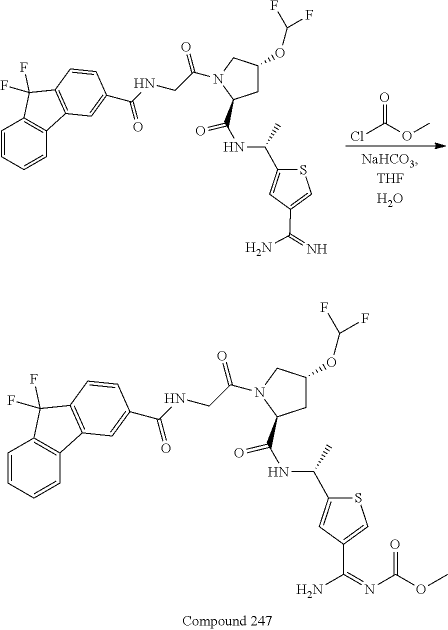

Compound 247 was prepared from (2S,4R)—N—((R)-1-(4-carbamimidoylthiophen-2-yl)ethyl)-1-((9,9-difluoro-9H-fluorene-3-carbonyl)glycyl)-4-(difluoromethoxy)pyrrolidine-2-carboxamide and methyl carbochlorination based on the procedures set forth in Scheme 143. ¹H NMR (400 MHz, CD₃OD) δ 8.46 (s, 1H), 8.17 (d, J=11.4 Hz, 1H), 8.07 (d, J=8.1 Hz, 1H), 7.89 (d, J=7.7 Hz, 1H), 7.74 (dd, J=14.9, 9.6 Hz, 3H), 7.65 (d, J=7.5 Hz, 1H), 7.56 (d, J=7.7 Hz, 1H), 7.44 (t, J=7.4 Hz, 1H), 6.74-6.29 (m, 1H), 5.28 (dd, J=41.7, 7.0 Hz, 1H), 5.04 (s, 1H), 4.62 (t, J=8.0 Hz, 1H), 4.36 (d, J=16.7 Hz, 1H), 4.17 (d, J=16.7 Hz, 1H), 3.98 (dd, J=11.7, 4.5 Hz, 1H), 3.89 (d, J=12.6 Hz, 1H), 3.51 (d, J=19.2 Hz, 3H), 2.54-2.46 (m, 1H), 2.26 (d, J=5.5 Hz, 1H), 1.59 (t, J=16.5 Hz, 3H). LCMS (ESI) m/z: 675 (M+H)⁺.

### Scheme 238: (2S,4R)—N—((R)-1-(4-carbamimidoylthiophen-2-yl)ethyl)-1-((dibenzo[b,d]furan-2-carbonyl)glycyl)-4-phenoxypyrrolidine-2-carboxamide (Compound 248)

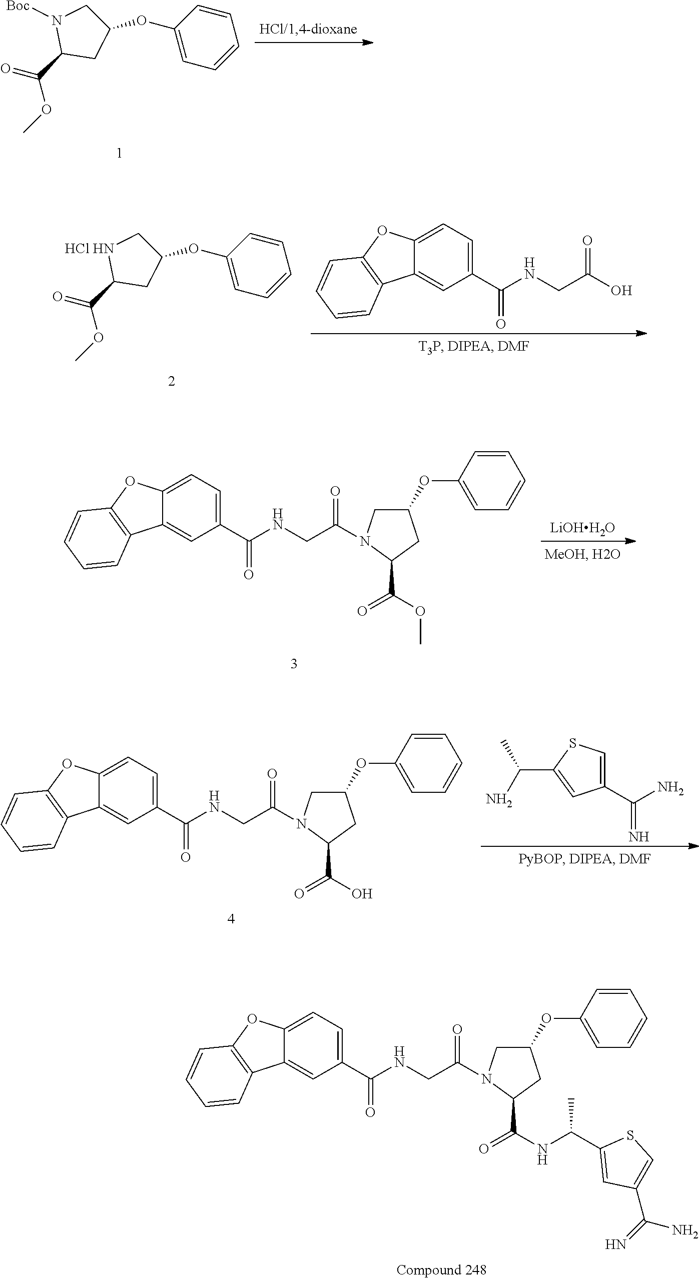

Compound 248 was prepared from 1-(tert-butyl) 2-methyl (2S,4R)-4-phenoxypyrrolidine-1,2-dicarboxylate, (dibenzo[b,d]furan-2-carbonyl)glycine, and (R)-5-(1-aminoethyl)thiophene-3-carboximidamide based on the procedures set forth in Scheme 3. ¹H NMR (400 MHz, CD₃OD) δ 8.57 (d, J=1.5 Hz, 1H), 8.52 (s, 1H), 8.22 (t, J=11.4 Hz, 1H), 8.10-7.99 (m, 2H), 7.65 (dd, J=11.4, 6.0 Hz, 2H), 7.58-7.52 (m, 2H), 7.43 (t, J=7.5 Hz, 1H), 7.31 (q, J=7.6 Hz, 2H), 6.98 (t, J=7.8 Hz, 3H), 5.26 (dd, J=24.0, 16.8 Hz, 2H), 4.64 (t, J=8.3 Hz, 1H), 4.25 (dt, J=23.2, 11.6 Hz, 2H), 4.11-3.95 (m, 2H), 2.59 (dd, J=13.8, 7.4 Hz, 1H), 2.28 (ddd, J=13.7, 8.7, 4.6 Hz, 1H), 1.58 (d, J=6.8 Hz, 3H). LCMS (ESI) (m/z): 610 (M+H)⁺.

### Scheme 239: Synthesis of hexyl ((E)-amino(5-((R)-1-((2S,4R)-1-((9,9-difluoro-9H-fluorene-3-carbonyl)glycyl)-4-fluoro-4-(methoxymethyl)pyrrolidine-2-carboxamido)ethyl)thiophen-3-yl)methylene)carbamate (Compound 249)

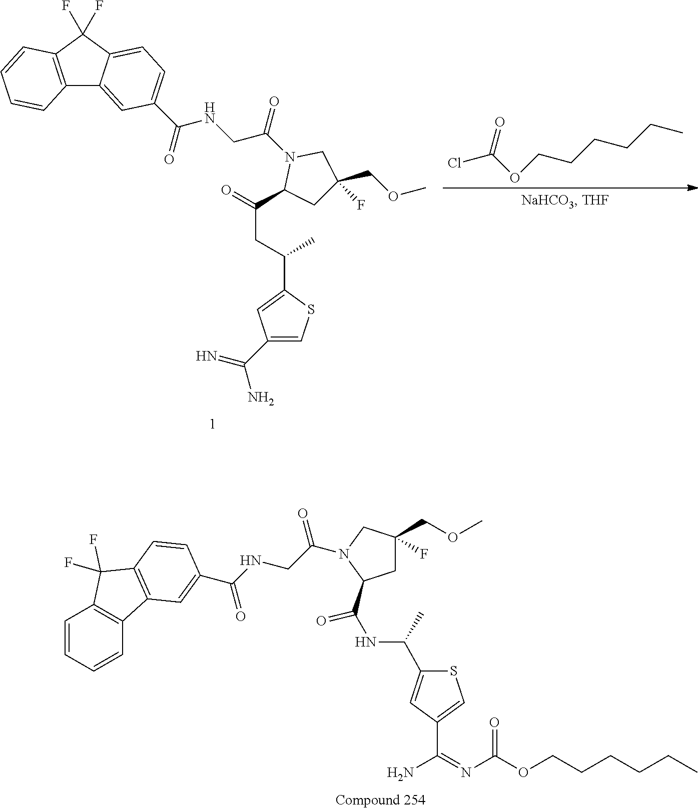

Compound 254 was prepared from (2S,4R)—N—((R)-1-(4-carbamimidoylthiophen-2-yl)ethyl)-1-((9,9-difluoro-9H-fluorene-3-carbonyl)glycyl)-4-fluoro-4-(methoxymethyl)pyrrolidine-2-carboxamide and hexyl carbochlorination based on the procedures set forth in Scheme 143. ¹H NMR (400 MHz, CD₃OD) δ 8.15 (d, J=11.6 Hz, 1H), 8.13-8.00 (m, 1H), 7.92-7.82 (m, 1H), 7.81-7.67 (m, 3H), 7.64 (d, J=6.8 Hz, 1H), 7.56 (t, J=6.8 Hz, 1H), 7.43 (t, J=7.4 Hz, 1H), 5.36-5.18 (m, 1H), 4.72-4.62 (m, 1H), 4.59-4.35 (m, 1H), 4.22-3.93 (m, 3H), 3.89-3.80 (m, 2H), 3.77-3.67 (m, 2H), 3.43 (d, J=8.4 Hz, 3H), 2.70-2.48 (m, 1H), 2.32-2.10 (m, 1H), 1.65-1.51 (m, 5H), 1.34-1.25 (m, 6H), 0.92-0.84 (m, 3H). LCMS (ESI) (m/z): 742 (M+H)⁺.

### Scheme 240: Synthesis of methyl ((E)-amino(5-((R)-1-((2S,4R)-1-((9,9-difluoro-9H-fluorene-3-carbonyl)glycyl)-4-fluoro-4-(fluoromethyl)pyrrolidine-2-carboxamido)ethyl)thiophen-3-yl)methylene)carbamate (Compound 250)

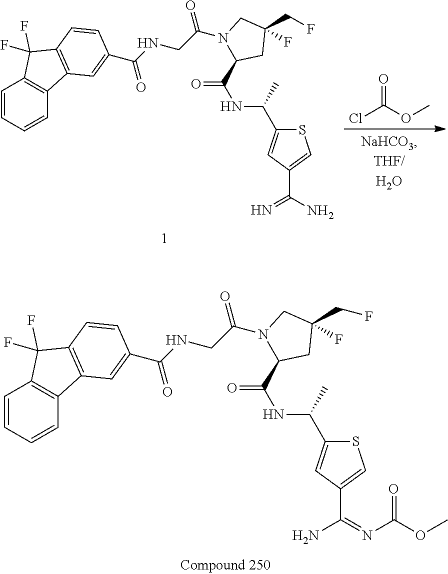

Compound 250 was prepared from (2S,4R)—N—((R)-1-(4-carbamimidoylthiophen-2-yl)ethyl)-1-((9,9-difluoro-9H-fluorene-3-carbonyl)glycyl)-4-fluoro-4-(fluoromethyl)pyrrolidine-2-carboxamide and methyl carbochlorination based on the procedures set forth in Scheme 143. ¹H NMR (400 MHz, CD₃OD) δ 8.16 (d, J=10.2 Hz, 1H), 8.08 (d, J=12.4 Hz, 1H), 7.88 (d, J=7.6 Hz, 1H), 7.74 (dd, J=12.5, 6.4 Hz, 3H), 7.65 (d, J=7.3 Hz, 1H), 7.59-7.55 (m, 1H), 7.44 (t, J=7.4 Hz, 1H), 5.23 (q, J=5.6 Hz, 1H), 4.69 (dd, J=18.0, 9.5 Hz, 2H), 4.40 (d, J=17.0 Hz, 1H), 4.18-4.09 (m, 2H), 3.98 (dd, J=32.1, 12.2 Hz, 1H), 3.67 (d, J=20.6 Hz, 1H), 3.53 (s, 3H), 2.69-2.55 (m, 1H), 2.31-2.15 (m, 1H), 1.60 (d, J=26.3 Hz, 3H). LC/MS (ESI) (m/z): 660 (M+H)⁺.

### Scheme 241: Synthesis of (S)—N-(2-(4-carbamimidoylthiophen-2-yl)ethyl)-7-((9,9-difluoro-9H-fluorene-3-carbonyl)glycyl)-1,4-dioxa-7-azaspiro[4.4]nonane-8-carboxamide (Compound 251)

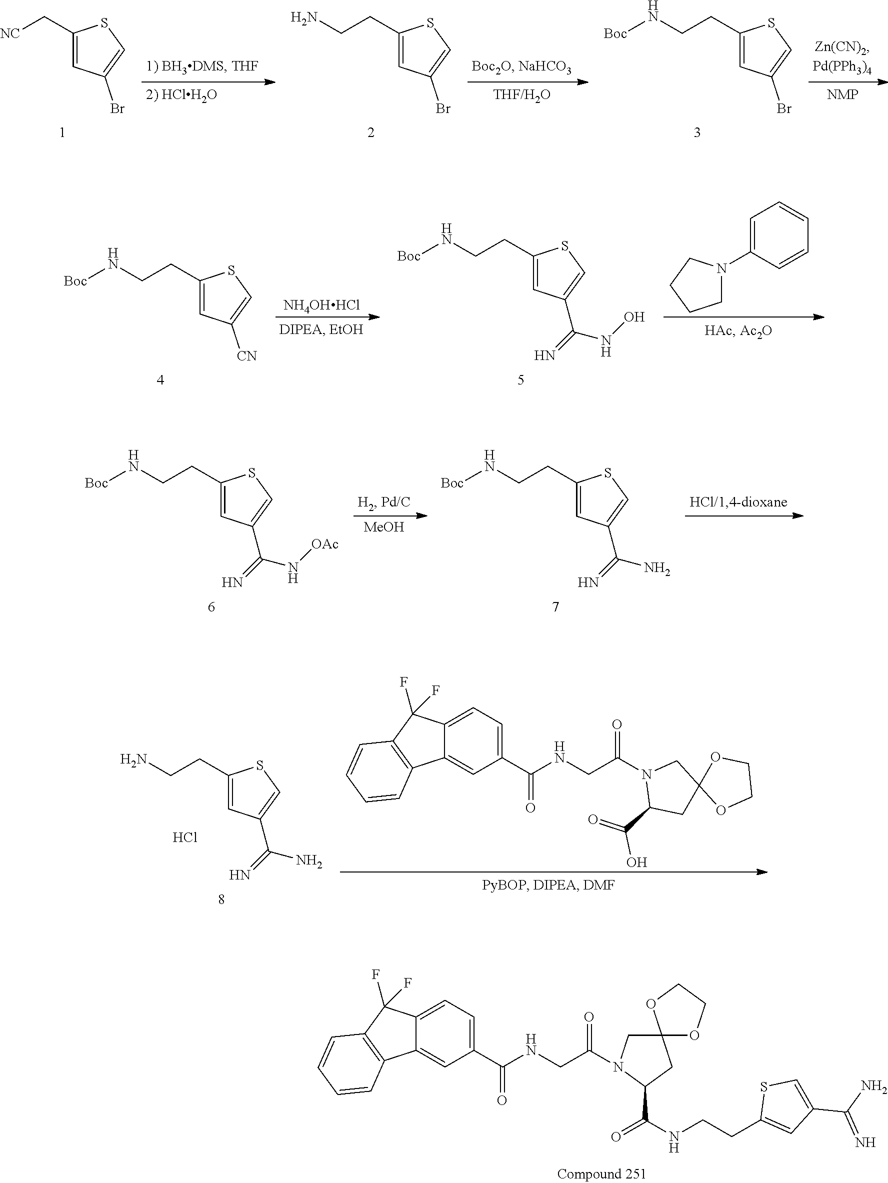

### Step 1: 2-(4-bromothiophen-2-yl)ethan-1-amine (2)

To a solution of 2-(4-bromothiophen-2-yl)acetonitrile (2 g, 10 mmol) in THF (20 mL) was added BH₃·DMS (5 mL, 10 M) dropwise and the solution was stirred at 90° C. under N₂ atmosphere for 2 hours. The mixture was quenched with HCl (10 mL, 3M). The mixture was used directly in the next step without further purification. LCMS (ESI) m/z: 206 (M+H)⁺.

### Step 2: tert-butyl (2-(4-bromothiophen-2-yl)ethyl)carbamate (3)

The mixture of previous step was basified with NaHCO₃ to pH ˜14, and Boc₂O (11.5 g, 52.8 mmol) was added. The reaction mixture was stirred at room temperature under N₂ atmosphere for overnight. The mixture was diluted with water and extracted with EtOAc twice. The combined organic layers were washed with brine, dried over anhydrous Na₂SO₄, filtered, and concentrated under reduced pressure to dryness. The residue was purified by flash column chromatography (silica gel, 0-50% EtOAc in PE) to give tert-butyl (2-(4-bromothiophen-2-yl)ethyl)carbamate (1.9 g, two steps yield 62.0%) as a yellow solid. LCMS (ESI) m/z: 306 (M+H)⁺.

### Step 3: tert-butyl (2-(4-cyanothiophen-2-yl)ethyl)carbamate (4)

To a mixture of tert-butyl (2-(4-bromothiophen-2-yl)ethyl)carbamate (1.9 g, 6.2 mmol) and Zn(CN)₂ (2.2 g, 18.6 mmol) in NMP (15 mL) was added Pd(PPh₃)₄ (855 mg, 0.74 mmol) at 25° C. under N₂ atmosphere and the reaction mixture was degassed under N₂ atmosphere for three times and stirred at 120° C. for 6 hours. The mixture was diluted with water and extracted with EtOAc twice. The combined organic layers were washed with brine, dried over anhydrous Na₂SO₄, filtered, and concentrated under reduced pressure to dryness. The residue was purified by flash column chromatography (silica gel, 0-50% EtOAc in PE) to give tert-butyl (2-(4-cyanothiophen-2-yl)ethyl)carbamate (1.3 g, yield 83.9%) as a yellow oil. LCMS (ESI) m/z: 253 (M+H)⁺.

### Step 4: tert-butyl (2-(4-(N-hydroxycarbamimidoyl)thiophen-2-yl)ethyl)carbamate (5)

To a solution of tert-butyl (2-(4-cyanothiophen-2-yl)ethyl)carbamate (1.3 g, 5.2 mmol) in EtOH (10 mL) was added NH₄OH·HCl (1.1 g, 15.6 mmol) and DIPEA (4.0 g, 31.2 mmol) and the reaction mixture was stirred at room temperature overnight. The mixture was diluted with water and extracted with EtOAc twice. The combined organic layers were washed with brine, dried over anhydrous Na₂SO₄, filtered, and concentrated under reduced pressure to dryness. The residue was purified by flash column chromatography (silica gel, 0-7% MeOH in DCM) to give tert-butyl (2-(4-(N-hydroxycarbamimidoyl) thiophen-2-yl)ethyl)carbamate (1.4 g, yield 96.2%) as a yellow oil. LCMS (ESI) m/z: 286 (M+H)⁺.

### Step 5: tert-butyl (2-(4-(N-acetoxycarbamimidoyl)thiophen-2-yl)ethyl)carbamate (6)

To a solution of tert-butyl (2-(4-(N-hydroxycarbamimidoyl)thiophen-2-yl)ethyl)carbamate (1.4 g, 5.0 mmol) in AcOH (5 mL) and Ac₂O (5 mL) was added 4-(pyrrolidin-1-yl)pyridine (10 mg) and the reaction solution was stirred for 10 minutes. The mixture was diluted with water and extracted with EtOAc twice. The combined organic layers were washed with brine, dried over anhydrous Na₂SO₄, filtered, and concentrated under reduced pressure to dryness. The residue was purified by flash column chromatography (silica gel, 0-40% EtOAc in PE) to give methyl (9,9-difluoro-5-methyl-9H-fluorene-3-carbonyl)glycinate (1.6 g, yield 97.8%) as a yellow solid. LCMS (ESI) m/z: 328 (M+H)⁺.

### Step 6: tert-butyl (2-(4-carbamimidoylthiophen-2-yl)ethyl)carbamate (7)

To a solution of tert-butyl (2-(4-(N-acetoxycarbamimidoyl)thiophen-2-yl)ethyl)carbamate (1.6 g, 1.4 mmol) in MeOH (10 mL) was added Pd/C (300 mg, 10% wt.), and the mixture was degassed under N₂ atmosphere for three times and stirred under a H₂ balloon at 25° C. overnight. The mixture was filtered, and the filtrate was concentrated to dryness under reduced pressure to give tert-butyl (2-(4-carbamimidoylthiophen-2-yl)ethyl)carbamate (1.3 g, yield 98.8%) as a white solid, which was used directly in the next step. LCMS (ESI) (m/z): 300 (M+H)⁺.

### Step 7: 5-(2-aminoethyl)thiophene-3-carboximidamide hydrochloride (8)

A mixture of tert-butyl (2-(4-carbamimidoylthiophen-2-yl)ethyl)carbamate (1.3 g, 4.8 mmol) in HCl/1,4-dioxane (10 mL) was stirred under N₂ atmosphere at room temperature for 2 hours. The reaction mixture was concentrated to dryness under reduced pressure, co-evaporate with DCM again and dried under vacuum to give 5-(2-aminoethyl)thiophene-3-carboximidamide hydrochloride (800 mg, yield 81.1%), which was used directly in the next step without further purification. LCMS (ESI) m/z: 170 (M+H)⁺.

### Step 8: (S)—N-(2-(4-carbamimidoylthiophen-2-yl)ethyl)-7-((9,9-difluoro-9H-fluorene-3-carbonyl)glycyl)-1,4-dioxa-7-azaspiro[4.4]nonane-8-carboxamide (Compound 251)

To a mixture of 5-(2-aminoethyl)thiophene-3-carboximidamide (24 mg, 0.14 mmol) and (S)-7-((9,9-difluoro-9H-fluorene-3-carbonyl)glycyl)-1,4-dioxa-7-azaspiro[4.4]nonane-8-carboxylic acid (40 mg, 0.087 mmol) in DMF (0.7 mL) was added PyBop (50 mg, 0.096 mmol) and DIPEA (67 mg, 0.52 mmol) under N₂ atmosphere at room temperature and stirred for 2 hours. The mixture was diluted with EtOAc, washed with water and brine, dried over anhydrous Na₂SO₄, filtered, and concentrated to dryness under reduced pressure. The residue was purified by chromatography (silica gel, 10% MeOH in DCM) and further purified by prep-HPLC to give Compound 251 (4.4 mg, yield 8.3%) as a white solid. ¹H NMR (400 MHz, CD₃OD) δ 8.54 (s, 1H), 8.15 (s, 1H), 8.00 (d, J=1.4 Hz, 1H), 7.86 (d, J=7.8 Hz, 1H), 7.77-7.72 (m, 2H), 7.66 (d, J=7.4 Hz, 1H), 7.58 (t, J=7.6 Hz, 1H), 7.46 (d, J=7.4 Hz, 1H), 7.32 (s, 1H), 4.95 (s, 1H), 4.27 (d, J=16.6 Hz, 1H), 4.13 (d, J=16.5 Hz, 1H), 4.00 (s, 4H), 3.81 (s, 2H), 3.59-3.54 (m, 1H), 3.43-3.39 (m, 1H), 3.12-3.07 (m, 2H), 2.41 (dd, J=13.1, 9.2 Hz, 1H), 2.20 (dd, J=13.3, 5.3 Hz, 1H). LCMS (ESI) m/z=610 (M+H)⁺.

### Scheme 242: Synthesis of (2S,4R)—N—((R)-1-(4-carbamimidoylthiophen-2-yl)ethyl)-1-((9,9-difluoro-9H-fluorene-3-carbonyl)glycyl)-4-((S)—S-methylsulfonimidoyl)pyrrolidine-2-carboxamide (Compound 252)

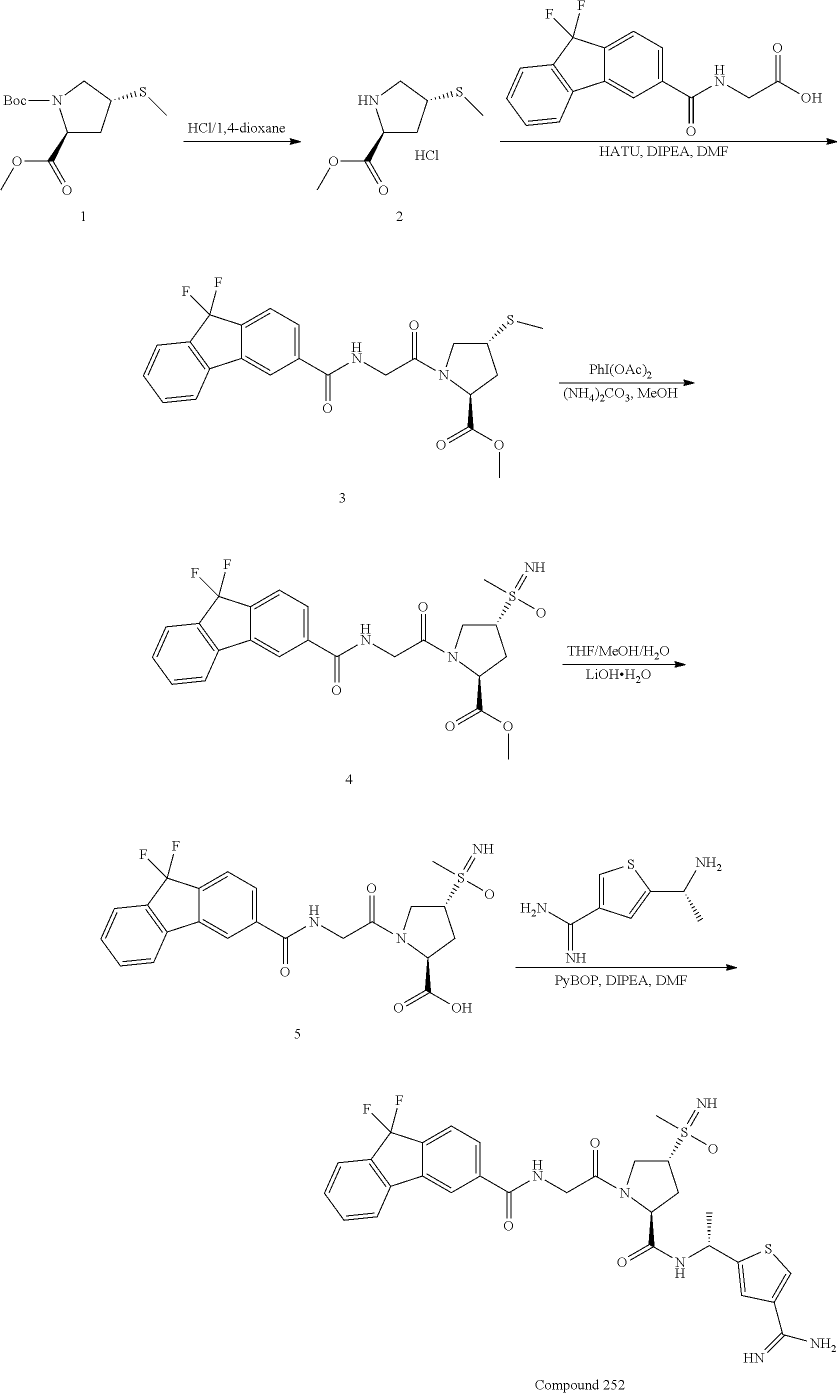

### Step 1: methyl (2S,4R)-4-(methylthio)pyrrolidine-2-carboxylate hydrochloride (2)

A solution of 1-(tert-butyl) 2-methyl (2S,4R)-4-(methylthio)pyrrolidine-1,2-dicarboxylate (140 mg, 0.51 mmol) in HCl/1,4-dioxane (2 mL, 4 M) was stirred under N₂ atmosphere at room temperature for 30 minutes. The reaction mixture was concentrated to dryness under reduced pressure, co-evaporate with DCM again and dried under vacuum to give methyl (2S,4R)-4-(methylthio)pyrrolidine-2-carboxylate hydrochloride (87 mg, yield 80.7%) as a yellow solid which was used directly in the next step without further purification. LC/MS (ESI) m/z: 176 (M+H)⁺.

### Step 2: methyl (2S,4R)-1-((9,9-difluoro-9H-fluorene-3-carbonyl)glycyl)-4-(methylthio)pyrrolidine-2-carboxylate (3)

To a mixture of methyl (2S,4R)-4-(methylthio)pyrrolidine-2-carboxylate hydrochloride (87 mg, 0.50 mmol) and (9,9-difluoro-9H-fluorene-3-carbonyl)glycine (150 mg, 0.50 mmol) in DMF (1.0 mL) was added HATU (285 mg, 0.75 mmol) and DIPEA (387 mg, 3.0 mmol) under N₂ atmosphere at room temperature and the reaction mixture was stirred for 2 hours. The mixture was diluted with EtOAc, washed with water and brine, dried over anhydrous Na₂SO₄, filtered, and concentrated to dryness under reduced pressure. The residue was purified by flash chromatography (silica gel, 0-40% EtOAc in PE) to give methyl (2S,4R)-1-((9,9-difluoro-9H-fluorene-3-carbonyl)glycyl)-4-(methylthio)pyrrolidine-2-carboxylate (150 mg, yield 65.2%) as a yellow oil. LCMS (ESI) m/z=461 (M+H)⁺.

### Step 3: methyl (2S,4R)-1-((9,9-difluoro-9H-fluorene-3-carbonyl)glycyl)-4-((S)—S-methylsulfonimidoyl) pyrrolidine-2-carboxylate (4)

To a mixture of methyl (2S,4R)-1-((9,9-difluoro-9H-fluorene-3-carbonyl)glycyl)-4-(methylthio)pyrrolidine-2-carboxylate (160 mg, 0.35 mmol) and PhI(OAc)₂ (258 mg, 0.80 mmol) in MeOH (3.5 mL) was added (NH₄)₂CO₃ (51 mg, 0.53 mmol) under N₂ atmosphere at room temperature and stirred for 4 hours. The mixture was diluted with EtOAc, washed with water and brine, dried over anhydrous Na₂SO₄, filtered, and concentrated to dryness under reduced pressure. The residue was purified by flash chromatography (silica gel, 0-40% EtOAc in PE) to give methyl (2S,4R)-1-((9,9-difluoro-9H-fluorene-3-carbonyl)glycyl)-4-((S)—S-methylsulfonimidoyl)pyrrolidine-2-carboxylate (70 mg, yield 40.0%) as a yellow oil. LCMS (ESI) m/z=492 (M+H)⁺.

### Step 4: (2S,4R)-1-((9,9-difluoro-9H-fluorene-3-carbonyl)glycyl)-4-((S)—S-methylsulfonimidoyl) pyrrolidine-2-carboxylic acid (5)

To a solution of methyl (2S,4R)-1-((9,9-difluoro-9H-fluorene-3-carbonyl)glycyl)-4-((S)—S-methylsulfonimidoyl)pyrrolidine-2-carboxylate (65 mg, 0.13 mmol) in MeOH (1.0 mL) and water (1 mL) was added LiOH·H₂O (33 mg, 0.78 mmol) at 0° C. and the mixture was stirred at room temperature for 1 hour. The mixture was acidified with 1 N aq·HCl to pH ˜3, extracted with EtOAc twice. The combined organic layers were washed with brine, dried over anhydrous Na₂SO₄, filtered, and concentrated under reduced pressure to dryness to give (2S,4R)-1-((9,9-difluoro-9H-fluorene-3-carbonyl)glycyl)-4-((S)—S-methylsulfonimidoyl)pyrrolidine-2-carboxylic acid (45 mg, yield 72.3%) as a white solid, which was used directly in the next step. LCMS (ESI) (m/z): 478 (M+H)⁺.

### Step 5: (2S,4R)—N—((R)-1-(4-carbamimidoylthiophen-2-yl)ethyl)-1-((9,9-difluoro-9H-fluorene-3-carbonyl) glycyl)-4-((S)—S-methylsulfonimidoyl)pyrrolidine-2-carboxamide (Compound 252)

To a mixture of (2S,4R)-1-((9,9-difluoro-9H-fluorene-3-carbonyl)glycyl)-4-((S)—S-methylsulfonimidoyl) pyrrolidine-2-carboxylic acid (45 mg, 0.094 mmol) and (R)-5-(1-aminoethyl)thiophene-3-carboximidamide (32 mg, 0.19 mmol) in DMF (1.0 mL) was added DIPEA (60 mg, 0.47 mmol) and PyBOP (49 mg, 0.094 mmol) at 0° C. and the mixture was stirred under N₂ atmosphere at room temperature for 2 hours. The mixture was diluted with EtOAc, washed with water and brine, dried over anhydrous Na₂SO₄, filtered, and concentrated to dryness under reduced pressure. The residue was purified by chromatography on silica gel (10% MeOH in DCM) and further purified by prep-HPLC to give Compound 252 (2.6 mg, yield 4.4%) as a white solid. ¹H NMR (400 MHz, CD₃OD) δ 8.52 (s, 1H), 8.20 (d, J=10.5 Hz, 2H), 7.88 (d, J=8.2 Hz, 1H), 7.78-7.71 (m, 2H), 7.65 (d, J=7.5 Hz, 1H), 7.57 (d, J=7.5 Hz, 1H), 7.49-7.44 (m, 2H), 5.27 (d, J=7.5 Hz, 1H), 4.75 (s, 1H), 4.37-4.25 (m, 2H), 4.19 (s, 3H), 3.08 (d, J=15.8 Hz, 3H), 2.73 (s, 1H), 2.45 (s, 1H), 1.60 (d, J=6.9 Hz, 3H). LCMS (ESI) (m/z): 629 (M+H)⁺.

### Scheme 243: Synthesis of hexyl ((E)-amino(5-((R)-1-((2S,4R)-1-((9,9-difluoro-9H-fluorene-3-carbonyl)glycyl)-4-fluoro-4-(fluoromethyl)pyrrolidine-2-carboxamido)ethyl)thiophen-3-yl) methylene)carbamate (Compound 253)

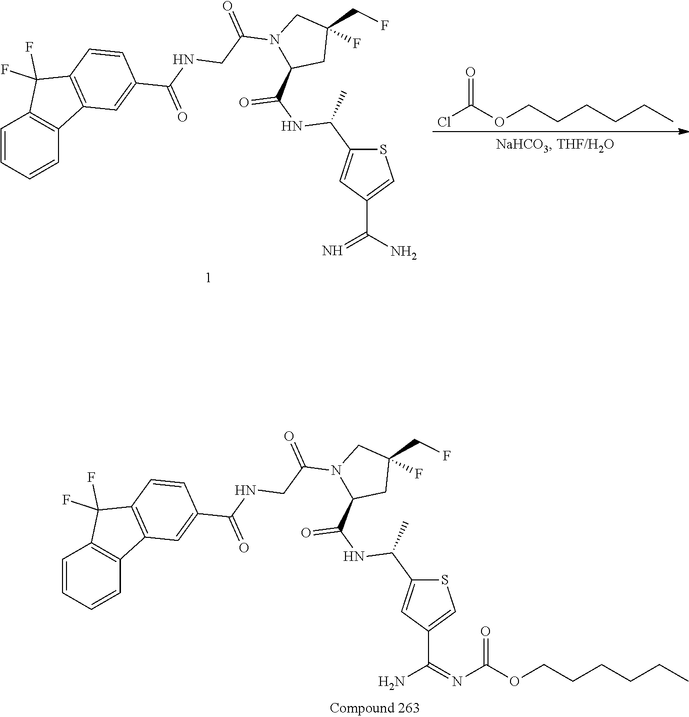

Compound 253 was prepared from (2S,4R)—N—((R)-1-(4-carbamimidoylthiophen-2-yl)ethyl)-1-((9,9-difluoro-9H-fluorene-3-carbonyl)glycyl)-4-fluoro-4-(fluoromethyl)pyrrolidine-2-carboxamide and hexyl carbochlorination based on the procedures set forth in Scheme 143. ¹H NMR (400 MHz, CD₃OD) δ 8.20-8.06 (m, 2H), 7.91-7.84 (m, 1H), 7.74 (dd, J=11.9, 6.1 Hz, 3H), 7.65 (d, J=7.6 Hz, 1H), 7.57 (t, J=6.3 Hz, 1H), 7.44 (t, J=7.5 Hz, 1H), 5.34-5.19 (m, 1H), 4.81-4.76 (m, 1H), 4.69 (dd, J=18.5, 11.0 Hz, 2H), 4.41 (d, J=17.0 Hz, 1H), 4.13 (dd, J=18.0, 12.3 Hz, 2H), 4.04-3.95 (m, 1H), 3.94-3.80 (m, 2H), 2.72-2.53 (m, 1H), 2.32-2.14 (m, 1H), 1.66-1.53 (m, 5H), 1.34-1.27 (m, 6H), 0.90 (t, J=7.0 Hz, 3H). LCMS (ESI) m/z: 716 (M+H)⁺.

### Scheme 244: Synthesis of (S)—N—((R)-1-(4-carbamimidoylthiophen-2-yl)ethyl)-7-((dibenzo[b,d]thiophene-2-carbonyl)glycyl)-1,4-dioxa-7-azaspiro[4.4]nonane-8-carboxamide (Compound 254)

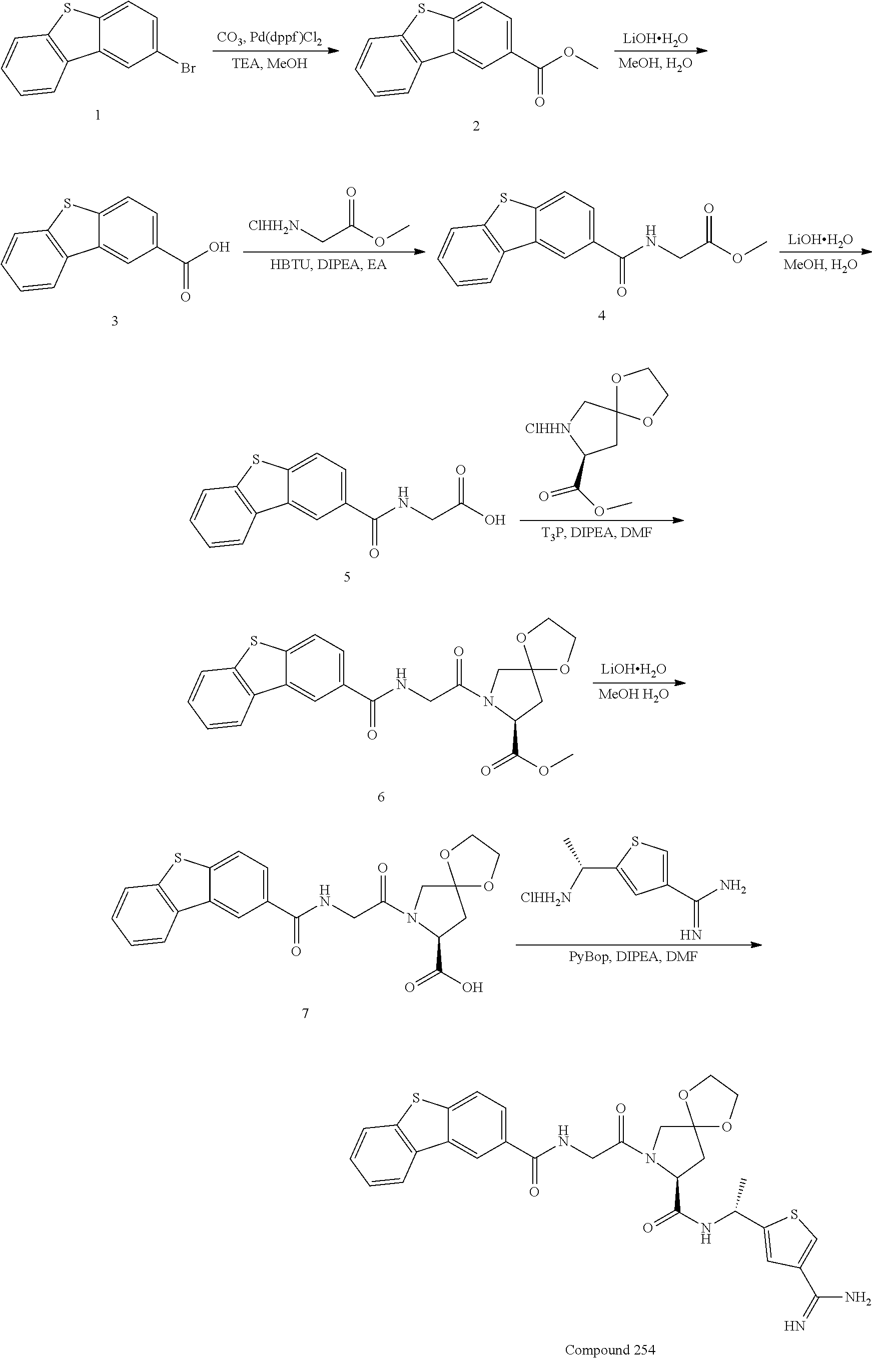

Compound 254 was prepared from 2-bromodibenzo[b,d]thiophene methyl glycinate hydrochloride, methyl (S)-1,4-dioxa-7-azaspiro[4.4]nonane-8-carboxylate, and (R)-5-(1-aminoethyl)thiophene-3-carboximidamide hydrochloride based on the procedures set forth in Scheme 66. ¹H NMR (400 MHz, CD₃OD) δ 8.79 (s, 1H), 8.29 (d, J=3.4 Hz, 1H), 8.23 (d, J=16.7 Hz, 1H), 8.01-7.94 (m, 3H), 7.54 (dd, J=6.3, 3.0 Hz, 3H), 5.36-5.24 (m, 1H), 4.58 (d, J=7.0 Hz, 1H), 4.29-4.17 (m, 2H), 4.04-3.96 (m, 4H), 3.83 (q, J=10.7 Hz, 2H), 2.49-2.40 (m, 1H), 2.22 (dd, J=13.1, 6.7 Hz, 1H), 1.59 (d, J=6.9 Hz, 3H). LCMS (ESI) (m/z): 592 (M+H)⁺.

### Scheme 245: (S)—N-((4-carbamimidoylthiophen-2-yl)methyl)-7-(2-(6-(4-fluorophenyl)-1-oxo-3,4-dihydroisoquinolin-2(1H)-yl)acetyl)-1,4-dioxa-7-azaspiro[4.4]nonane-8-carboxamide (Compound 255)

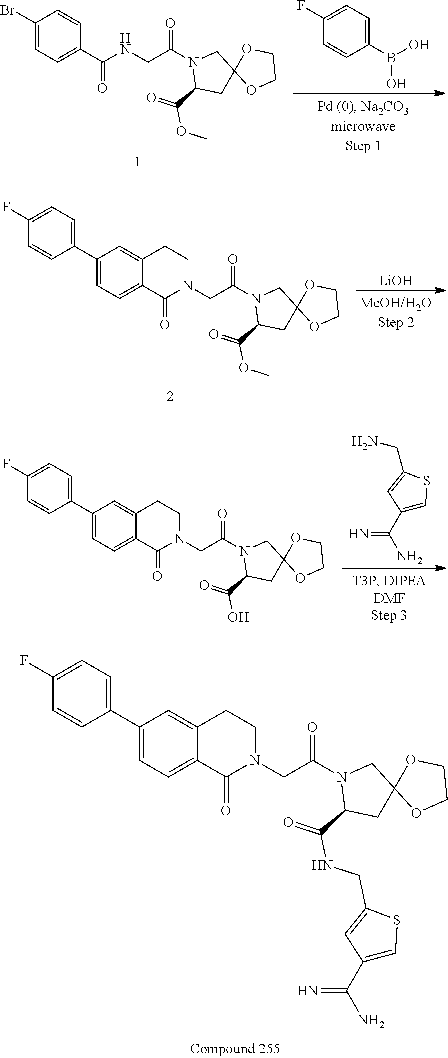

Compound 255 was prepared from (4-fluorophenyl)boronic acid, methyl (8S)-7-[2-(6-bromo-1-oxo-3,4-dihydroisoquinolin-2-yl)acetyl]-1,4-dioxa-7-azaspiro[4.4]nonane-8-carboxylate, and 5-(aminomethyl)thiophene-3-carboximidamide. HCl based on the procedures set forth in Scheme 199. LC/MS (ESI) m/z: 421 (M+H)⁺. ¹H NMR (400 MHz, DMSO) δ 8.60 (s, 1H), 8.32 (s, 1H), 8.13 (s, 2H), 7.91 (q, J=7.0 Hz, 1H), 7.84 (d, J=7.9 Hz, 1H), 7.76 (dd, J=8.2, 6.2 Hz, 2H), 7.70 (s, 1H), 7.64-7.51 (m, 1H), 7.25 (t, J=8.5 Hz, 2H), 7.18-7.00 (m, 2H), 4.52-4.28 (m, 2H), 4.14 (d, J=16.4 Hz, 2H), 3.92-3.81 (m, 3H), 3.73-3.58 (m, 2H), 3.55-3.51 (m, 2H), 3.06-2.98 (m, 2H), 2.26 (dd, J=13.0, 9.2 Hz, 2H), 2.03-1.94 (m, 1H), 1.47-1.39 (m, 1H)

### Scheme 246: (S)—N-((4-carbamimidoylthiophen-2-yl)methyl)-7-(2-(1-oxo-6-phenyl-3,4-dihydroisoquinolin-2(1H)-yl)acetyl)-1,4-dioxa-7-azaspiro[4.4]nonane-8-carboxamide (Compound 256)

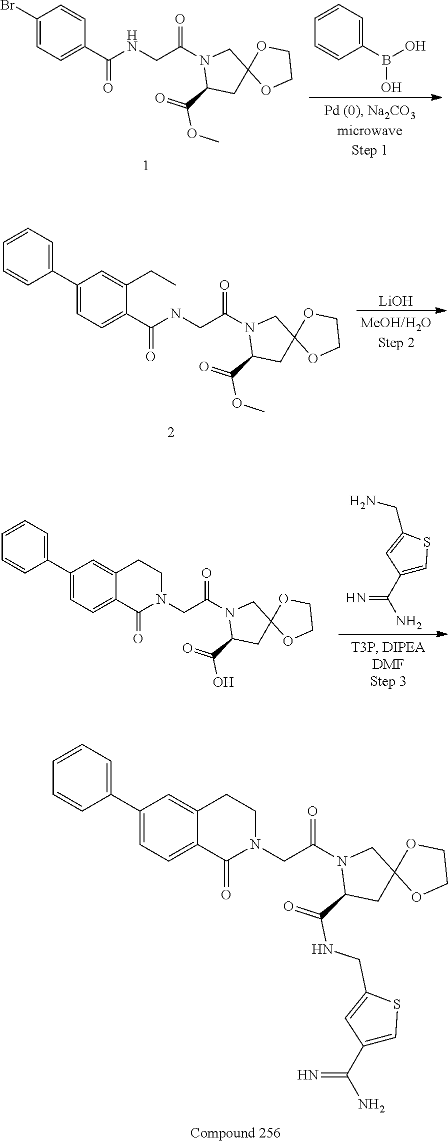

Compound 256 was prepared from phenylboronic acid, methyl (8S)-7-[2-(6-bromo-1-oxo-3,4-dihydroisoquinolin-2-yl)acetyl]-1,4-dioxa-7-azaspiro[4.4]nonane-8-carboxylate, and 5-(aminomethyl)thiophene-3-carboximidamide. HCl based on the procedures set forth in Scheme 199. LC/MS (ESI) m/z: 421 (M+H)+. ¹H NMR (400 MHz, DMSO) δ 8.65 (s, 1H), 8.38 (s, 1H), 7.92 (d, J=8.0 Hz, 1H), 7.73 (d, J=7.4 Hz, 2H), 7.68-7.60 (m, 2H), 7.50 (t, J=7.5 Hz, 2H), 7.41 (t, J=7.0 Hz, 1H), 7.12 (s, 1H), 4.53 (d, J=16.7 Hz, 2H), 4.39 (t, J=7.8 Hz, 1H), 4.20 (d, J=16.5 Hz, 1H), 3.94 (d, J=17.7 Hz, 3H), 3.76 (d, J=10.7 Hz, 1H), 3.73-3.62 (m, 1H), 3.60-3.55 (m, 2H), 3.06-3,01 (m, 2H), 2.99 (p, J=6.6 Hz, 2H), 2.44 (t, J=7.0 Hz, 1H), 2.07-2.0 (m, 1H).

### Scheme 247: (S)—N-((4-carbamimidoylthiophen-2-yl)methyl)-7-((2′,4′-dimethyl-[1,1′-biphenyl]-4-carbonyl)glycyl)-1,4-dioxa-7-azaspiro[4.4]nonane-8-carboxamide (Compound 257)

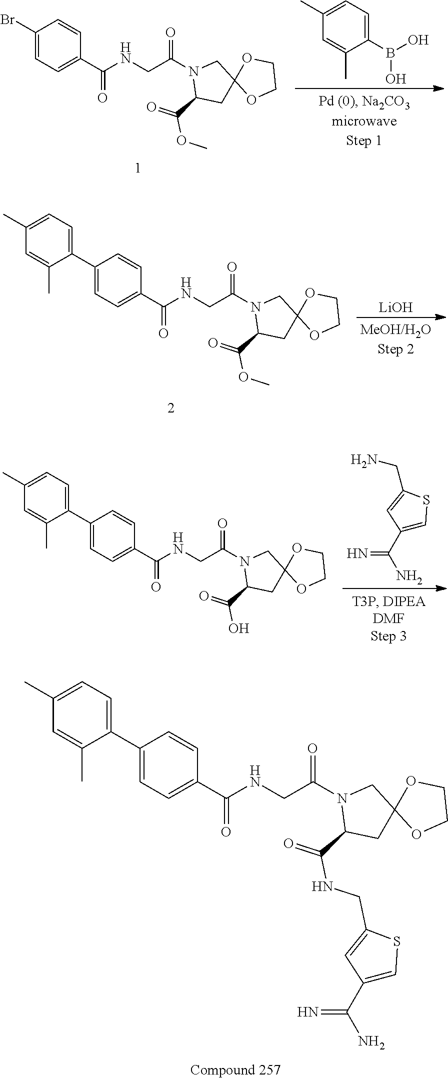

Compound 257 was prepared from (2,4-dimethylphenyl)boronic acid, methyl (8S)-7-[2-[(4-bromobenzoyl)amino]acetyl]-1,4-dioxa-7-azaspiro[4.4]nonane-8-carboxylate, and 5-(aminomethyl)thiophene-3-carboximidamide. HCl based on the procedures set forth in Scheme 199. LC/MS (ESI) m/z: 576 (M+H)⁺. ¹H NMR (400 MHz, DMSO) δ 8.70 (d, J=6.9 Hz, 1H), 8.65 (s, 1H), 8.43 (s, 1H), 7.91 (d, J=7.9 Hz, 2H), 7.82-7.71 (m, 2H), 7.41 (d, J=7.8 Hz, 2H), 7.10 (dd, J=14.9, 8.0 Hz, 2H), 4.56-4.48 (m, 3H), 4.40 (t, J=7.8 Hz, 1H), 4.19 (dd, J=16.8, 5.5 Hz, 1H), 3.95 (tt, J=17.4, 6.5 Hz, 4H), 3.81 (d, J=10.8 Hz, 1H), 3.74-3.60 (m, 1H), 2.32 (s, 3H), 2.21 (s, 3H), 2.07-1.99 (m, 2H).

### Scheme 248: Synthesis of hexyl ((E)-amino(5-((R)-1-((2S,4R)-1-((9,9-difluoro-9H-fluorene-3-carbonyl)glycyl)-4-(difluoromethoxy)pyrrolidine-2-carboxamido)ethyl) thiophen-3-yl)methylene)carbamate (Compound 258)

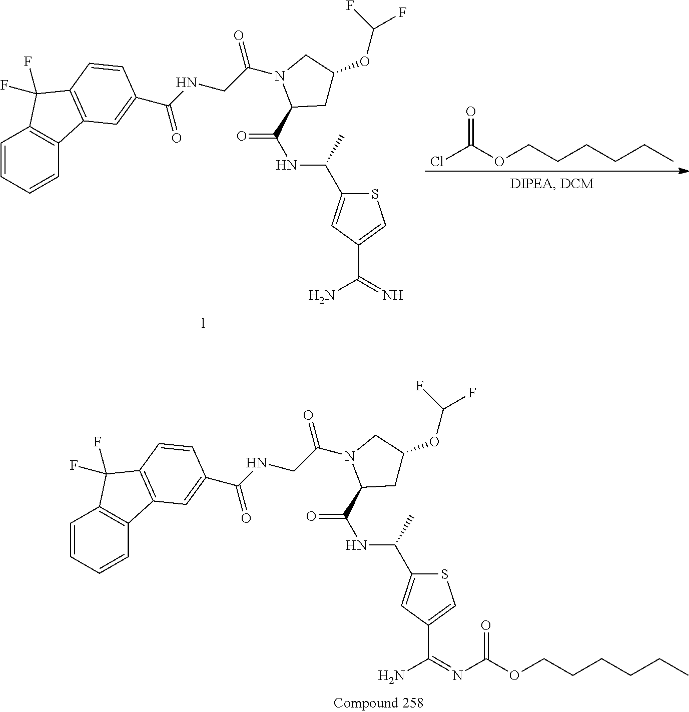

Compound 258 was prepared from (2S,4R)—N—((R)-1-(4-carbamimidoylthiophen-2-yl)ethyl)-1-((9,9-difluoro-9H-fluorene-3-carbonyl)glycyl)-4-(difluoromethoxy)pyrrolidine-2-carboxamide and hexyl carbonochloridate based on the procedures set forth in Scheme 143. ¹H NMR (400 MHz, CD₃OD) δ 8.16 (d, J=10.3 Hz, 1H), 8.07 (d, J=11.1 Hz, 1H), 7.88 (d, J=7.8 Hz, 1H), 7.72 (M, 3H), 7.64 (d, J=7.3 Hz, 1H), 7.56 (t, J=7.0 Hz, 1H), 7.43 (t, J=7.4 Hz, 1H), 6.73-6.30 (m, 1H), 5.33-5.19 (m, 1H), 5.05 (s, 1H), 4.62 (t, J=7.8 Hz, 1H), 4.36 (d, J=16.8 Hz, 1H), 4.16 (d, J=16.8 Hz, 1H), 4.01-3.96 (m, 1H), 3.94-3.80 (m, 3H), 2.54-2.40 (m, 1H), 2.32-2.19 (m, 1H), 1.58 (t, J=17.4 Hz, 5H), 1.34-1.26 (m, 6H), 0.90 (t, J=6.6 Hz, 3H). LCMS (ESI) m/z: 746 (M+H)⁺.

### Scheme 249: Synthesis of (1S,3S,5S)—N-(4-carbamimidoylthiophen-2-yl)-2-((9,9-difluoro-9H-fluorene-3-carbonyl)glycyl)-5-methyl-2-azabicyclo[3.1.0]hexane-3-carboxamide (Compound 259)

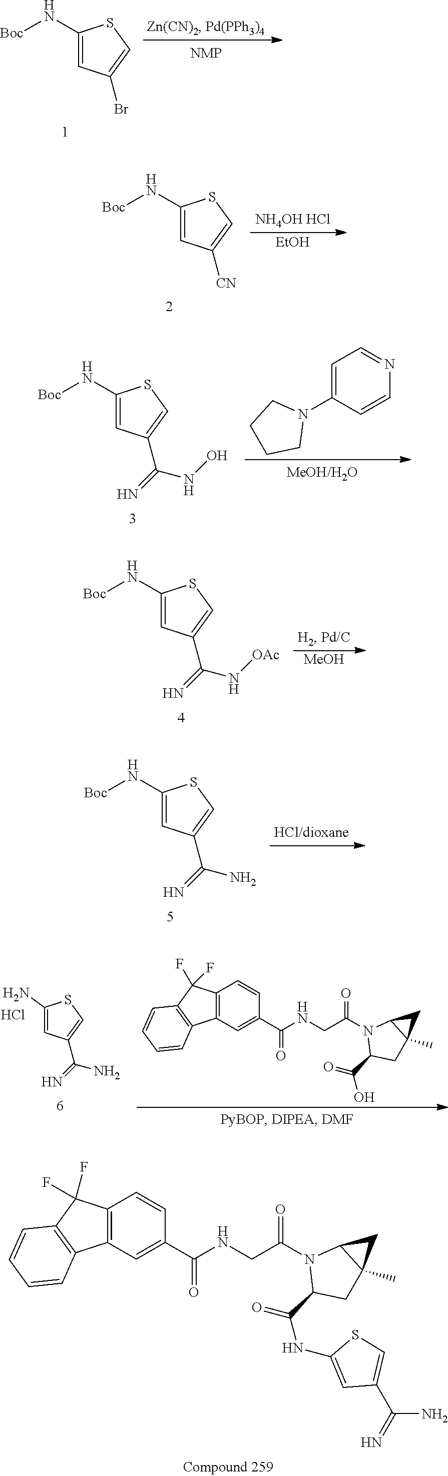

### Step 1: tert-butyl (4-cyanothiophen-2-yl)carbamate (2)

To a mixture of tert-butyl (4-bromothiophen-2-yl)carbamate (430 mg, 1.6 mmol) and Zn(CN)₂ (538 mg, 4.6 mmol) in NMP (4 mL) was added Pd(PPh₃)₄ (220 mg, 0.19 mmol), degassed under N₂ atmosphere and stirred at 120° C. for 6 hours. The mixture was diluted with water and extracted with EtOAc twice. The combined organic layers were washed with brine, dried over anhydrous Na₂SO₄, filtered, and concentrated under reduced pressure to dryness. The residue was purified by flash column chromatography (silica gel, 0-50% EtOAc in PE) to give tert-butyl (4-cyanothiophen-2-yl)carbamate (170 mg, yield 49.0%) as a yellow oil. LCMS (ESI) m/z: 225 (M+H)⁺.

### Step 2: tert-butyl (2-(4-(N-hydroxycarbamimidoyl)thiophen-2-yl)ethyl)carbamate (3)

To a solution of tert-butyl (4-cyanothiophen-2-yl)carbamate (170 mg, 0.76 mmol) in EtOH (2 mL) was added NH₄OH·HCl (160 mg, 2.3 mmol) and DIPEA (593 mg, 4.6 mmol) and the reaction mixture was stirred at room temperature overnight. The mixture was diluted with water and extracted with EtOAc twice. The combined organic layers were washed with brine, dried over anhydrous Na₂SO₄, filtered, and concentrated under reduced pressure to dryness. The residue was purified by flash column chromatography (silica gel, 0-7% MeOH in DCM) to give tert-butyl (4-(N-hydroxycarbamimidoyl)thiophen-2-yl)carbamate (180 mg, yield 92.1%) as a yellow oil. LCMS (ESI) m/z: 258 (M+H)⁺.

### Step 3: tert-butyl (4-(N-acetoxycarbamimidoyl)thiophen-2-yl)carbamate (4)

To a solution of tert-butyl (4-(N-hydroxycarbamimidoyl)thiophen-2-yl)carbamate (180 mg, 0.70 mmol) in AcOH (2 mL) was added Ac₂O (2 mL) and 4-(pyrrolidin-1-yl)pyridine (5.2 mg, 0.04 mmol) and the reaction solution was stirred for 10 minutes. The mixture was diluted with water and extracted with EtOAc twice. The combined organic layers were washed with brine, dried over anhydrous Na₂SO₄, filtered, and concentrated under reduced pressure to dryness. The residue was purified by flash column chromatography (silica gel, 0-40% EtOAc in PE) to give tert-butyl (4-(N-acetoxycarbamimidoyl)thiophen-2-yl)carbamate (190 mg, yield 91.4%) as a yellow solid. LCMS (ESI) m/z: 300 (M+H)⁺.

### Step 4: tert-butyl (4-carbamimidoylthiophen-2-yl)carbamate (5)

To a solution of tert-butyl (4-(N-acetoxycarbamimidoyl)thiophen-2-yl)carbamate (190 mg, 0.64 mmol) in MeOH (4 mL) was added Pd/C (60 mg, 10% wt.) at room temperature and the mixture was degassed under N₂ atmosphere for three times and stirred under a H₂ balloon at 25° C. for 1 hour. The mixture was filtered, and the filtrate was concentrated to dryness under reduced pressure to give tert-butyl (4-carbamimidoylthiophen-2-yl)carbamate (149 mg, yield 96.9%) as a white solid, which was used directly in the next step. LCMS (ESI) (m/z): 242 (M+H)⁺.

### Step 5: 5-aminothiophene-3-carboximidamide hydrochloride (6)

To a mixture of tert-butyl (2-(4-carbamimidoylthiophen-2-yl)ethyl)carbamate (60 mg, 0.25 mmol) in HCl/1,4-dioxane (1 mL) was stirred under N₂ atmosphere at room temperature for 2 hours. The reaction mixture was concentrated to dryness under reduced pressure, co-evaporate with DCM again and dried under vacuum to give 5-aminothiophene-3-carboximidamide hydrochloride (32 mg, yield 92.0%), which was used directly in the next step without further purification. LCMS (ESI) m/z: 142 (M+H)⁺.

### Step 6: (1S,3S,5S)—N-(4-carbamimidoylthiophen-2-yl)-2-((9,9-difluoro-9H-fluorene-3-carbonyl)glycyl)-5-methyl-2-azabicyclo[3.1.0]hexane-3-carboxamide (Compound 259)

To a mixture of 5-aminothiophene-3-carboximidamide hydrochloride (32 mg, 0.23 mmol) and (1S,3S,5S)-2-((9,9-difluoro-9H-fluorene-3-carbonyl)glycyl)-5-methyl-2-azabicyclo[3.1.0]hexane-3-carboxylic acid (50 mg, 0.12 mmol) in DMF (0.7 mL) was added PyBOP (67 mg, 0.13 mmol) and DIPEA (90 mg, 0.70 mmol) under N₂ atmosphere at room temperature and stirred for 2 hours. The mixture was diluted with EtOAc, washed with water and brine, dried over anhydrous Na₂SO₄, filtered, and concentrated to dryness under reduced pressure. The residue was purified by chromatography (silica gel, 0-10% MeOH in DCM) and further purified by prep-HPLC to give Compound 259 (2.5 mg, yield 3.9%) as a white solid. ¹H NMR (400 MHz, CDCl₃) δ 8.02 (d, J=13.2 Hz, 1H), 7.78 (dd, J=15.6, 7.8 Hz, 1H), 7.68-7.62 (m, 3H), 7.51 (d, J=10.1 Hz, 1H), 7.38 (d, J=7.7 Hz, 2H), 7.22 (s, 1H), 6.48 (s, 1H), 5.00 (d, J=13.8 Hz, 1H), 4.33 (d, J=123.0 Hz, 2H), 3.91 (s, 2H), 3.41 (d, J=236.7 Hz, 1H), 2.45 (dd, J=30.5, 19.0 Hz, 2H), 1.31 (s, 3H), 0.90 (d, J=16.4 Hz, 1H), 0.72 (d, J=36.8 Hz, 1H). LCMS (ESI) m/z=550 (M+H)⁺.

### Scheme 250: Synthesis of (2S,4R)-4-((1H-1,2,3-triazol-1-yl)methyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (Compound 260)

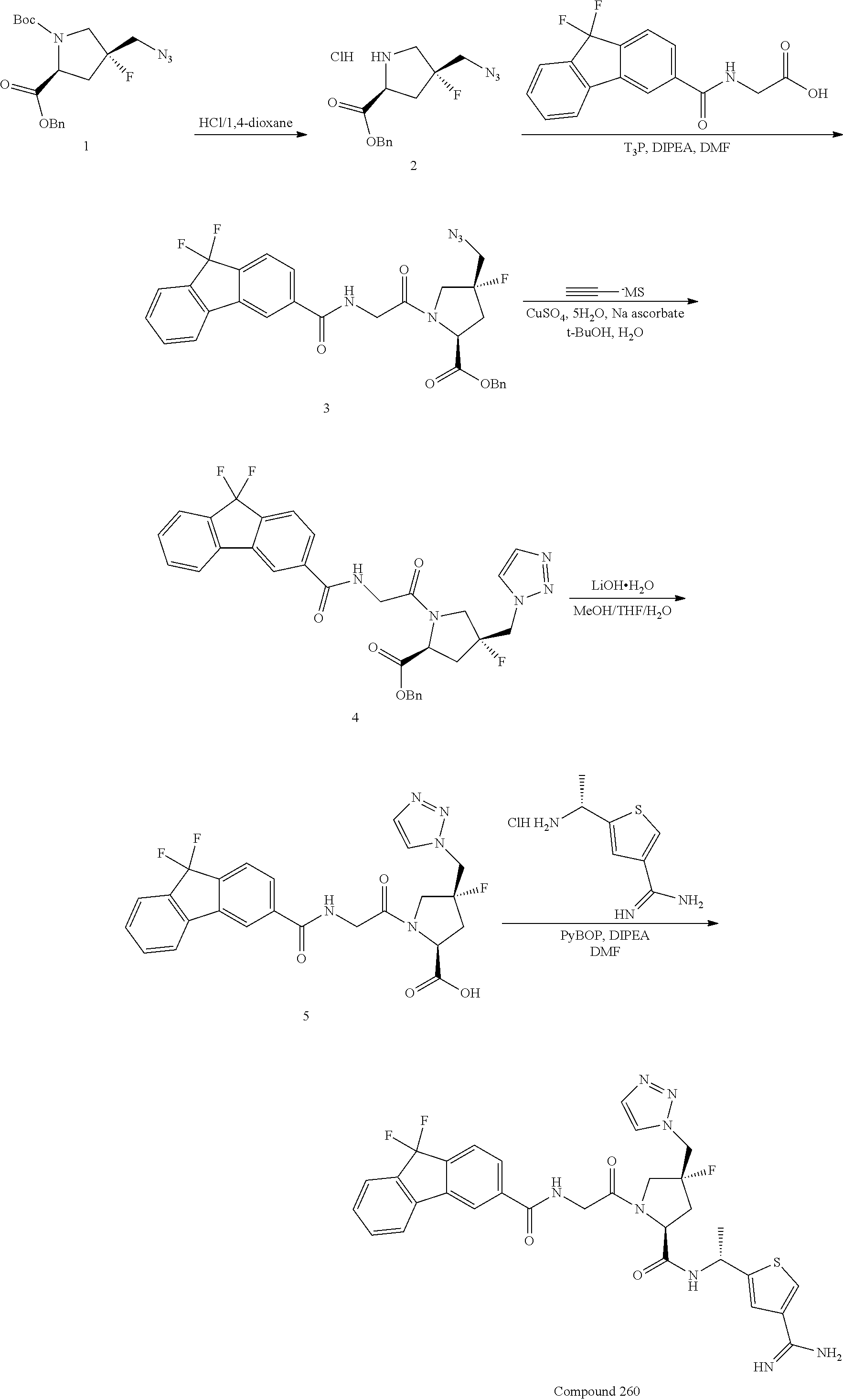

### Step 1: benzyl (2S,4R)-4-(azidomethyl)-4-fluoropyrrolidine-2-carboxylate hydrochloride (2)

A solution of 2-benzyl 1-(tert-butyl) (2S,4R)-4-(azidomethyl)-4-fluoropyrrolidine-1,2-dicarboxylate (100 mg, 0.26 mmol) in HCl/1,4-dioxane (2 mL, 4M) was stirred at room temperature for 1 hour. The mixture was concentrated to dryness under reduced pressure to give benzyl (2S,4R)-4-(azidomethyl)-4-fluoropyrrolidine-2-carboxylate hydrochloride (80 mg, yield 96.2%) as a white solid, which was directly used in the next reaction without further purification. LC/MS (ESI) m/z: 279 (M+H)⁺.

### Step 2: benzyl (2S,4R)-4-(azidomethyl)-1-((9,9-difluoro-9H-fluorene-3-carbonyl) glycyl)-4-fluoropyrrolidine-2-carboxylate (3)

To a mixture of benzyl (2S,4R)-4-(azidomethyl)-4-fluoropyrrolidine-2-carboxylate hydrochloride (80 mg, 0.25 mmol) and (9,9-difluoro-9H-fluorene-3-carbonyl)glycine (77 mg, 0.25 mmol) in DMF (3 mL) was added DIPEA (131 mg, 1.02 mmol) and T₃P (210 mg, 0.33 mmol, 50% wt. in EtOAc) under N₂ atmosphere and the mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-80% EtOAc in PE) to give benzyl (2S,4R)-4-(azidomethyl)-1-((9,9-difluoro-9H-fluorene-3-carbonyl)glycyl)-4-fluoropyrrolidine-2-carboxylate (100 mg, yield 69.2%) as a light oil. LCMS (ESI) m/z: 564 (M+H)⁺.

### Step 3: benzyl (2S,4R)-4-((1H-1,2,3-triazol-1-yl)methyl)-1-((9,9-difluoro-9H-fluorene-3-carbonyl)glycyl)-4-fluoropyrrolidine-2-carboxylate (4)

To a solution of benzyl (2S,4R)-4-(azidomethyl)-1-((9,9-difluoro-9H-fluorene-3-carbonyl)glycyl)-4-fluoropyrrolidine-2-carboxylate (100 mg, 0.18 mmol) in t-BuOH (1.8 mL) and H₂O (3.6 mL) was added trimethylsilyl acetylene (53 mg, 0.54 mmol), CuSO₄ (97 mg, 0.36 mmol) and sodium ascorbate (199 mg, 1.00 mmol) at 0° C. The mixture was stirred at 60° C. for 4 hours in a sealed tube. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-15% DCM in MeOH) to give benzyl (2S,4R)-4-((1H-1,2,3-triazol-1-yl)methyl)-1-((9,9-difluoro-9H-fluorene-3-carbonyl)glycyl)-4-fluoropyrrolidine-2-carboxylate (40 mg, yield 38.1%) as a white solid. LCMS (ESI) m/z: 590 (M+H)⁺.

### Step 4: (2S,4R)-4-((1H-1,2,3-triazol-1-yl)methyl)-1-((9,9-difluoro-9H-fluorene-3-carbonyl)glycyl)-4-fluoropyrrolidine-2-carboxylic acid (5)

To a solution of benzyl (2S,4R)-4-((1H-1,2,3-triazol-1-yl)methyl)-1-((9,9-difluoro-9H-fluorene-3-carbonyl)glycyl)-4-fluoropyrrolidine-2-carboxylate (40 mg, 0.068 mmol) in MeOH (2 mL) and THF (1 mL) was added a solution of LiOH·H₂O (11 mg, 0.27 mmol) in water (1 mL) at 0° C. and the mixture was stirred at room temperature for 1 hour. The mixture was diluted with water and extracted with EtOAc twice. The aqueous layer was acidified with 1 N aq·HCl to pH ˜4 and extracted with EtOAc twice. The combined organic layers were washed with brine, dried over anhydrous Na₂SO₄, filtered, and concentrated to dryness under reduced pressure to give (2S,4R)-4-((1H-1,2,3-triazol-1-yl)methyl)-1-((9,9-difluoro-9H-fluorene-3-carbonyl)glycyl)-4-fluoropyrrolidine-2-carboxylic acid (33 mg, yield 97.4%) as a light oil, which was used directly in the next step. LC/MS (ESI) m/z: 500 (M+H)⁺.

### Step 5: (2S,4R)-4-((1H-1,2,3-triazol-1-yl)methyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (Compound 260)

To a mixture of (2S,4R)-4-((1H-1,2,3-triazol-1-yl)methyl)-1-((9,9-difluoro-9H-fluorene-3-carbonyl)glycyl)-4-fluoropyrrolidine-2-carboxylic acid (33 mg, 0.068 mmol) and (R)-5-(1-aminoethyl)thiophene-3-carboximidamide hydrochloride (28 mg, 0.14 mmol) in DMF (3 mL) was added DIPEA (44 mg, 0.34 mmol) and PyBOP (43 mg, 0.082 mmol) at 0° C. and the mixture was stirred under N₂ atmosphere at room temperature for 1 hour. The mixture was diluted with EtOAc, washed with water and brine, dried over anhydrous Na₂SO₄, filtered, and concentrated to dryness under reduced pressure. The residue was purified by prep-TLC (DCM:MeOH=10:1) and further purified by prep-HPLC to give Compound 260 (8.0 mg, yield 18.1%) as a white solid. ¹H NMR (400 MHz, CD₃OD) δ 8.28-8.13 (m, 2H), 8.07 (d, J=10.4 Hz, 1H), 7.87 (d, J=7.6 Hz, 1H), 7.83-7.79 (m, 1H), 7.76 (d, J=7.6 Hz, 1H), 7.71 (d, J=7.8 Hz, 1H), 7.65 (d, J=7.2 Hz, 1H), 7.61-7.52 (m, 2H), 7.45 (t, J=7.6 Hz, 1H), 5.42-5.25 (m, 1H), 5.09-4.98 (m, 2H), 4.66-4.55 (m, 1H), 4.37-4.24 (m, 1H), 4.20-3.97 (m, 3H), 2.64-2.24 (m, 2H), 1.63 (dd, J=35.2, 35.2 Hz, 3H). LCMS (ESI) (m/z): 651 (M+H)⁺.

### Scheme 251: Synthesis of (S)—N—((R)-1-(4-carbamimidoylthiophen-2-yl)ethyl)-7-((5,5-dioxidodibenzo[b,d]thiophene-2-carbonyl)glycyl)-1,4-dioxa-7-azaspiro[4.4]nonane-8-carboxamide (Compound 261)

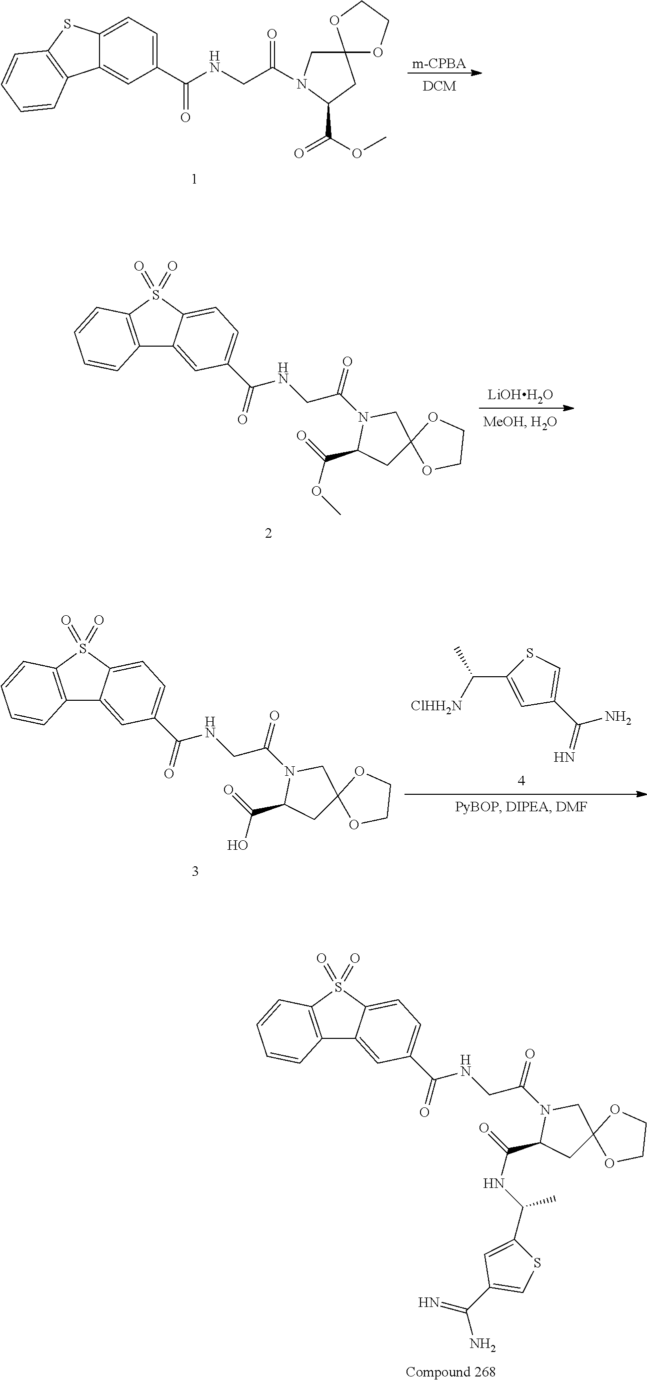

### Step 1: methyl (S)-7-((5,5-dioxidodibenzo[b,d]thiophene-2-carbonyl)glycyl)-1,4-dioxa-7-azaspiro[4.4]nonane-6-carboxylate (2)

To a mixture of methyl (S)-7-((dibenzo[b,d]thiophene-2-carbonyl)glycyl)-1,4-dioxa-7-azaspiro[4.4]nonane-8-carboxylate (130 mg, 0.29 mmol) in DCM (1.5 mL) was added m-CPBA (99 mg, 0.58 mmol) under N₂ atmosphere, and the mixture was stirred at room temperature for 4 hours. The mixture was concentrated under reduced pressure to dryness. The residue was purified by flash chromatography (silica gel, 0-70% EtOAc in PE) to give methyl (S)-7-((5,5-dioxidodibenzo[b,d]thiophene-2-carbonyl)glycyl)-1,4-dioxa-7-azaspiro[4.4]nonane-8-carboxylate (100 mg, yield 71.9%) as a yellow solid. LC/MS (ESI) (m/z): 487 (M+H)⁺.

### Step 2: (S)-7-((5,5-dioxidodibenzo[b,d]thiophene-2-carbonyl)glycyl)-1,4-dioxa-7-azaspiro[4.4]nonane-8-carboxylic acid (3)

To a solution of methyl (S)-7-((5,5-dioxidodibenzo[b,d]thiophene-2-carbonyl)glycyl)-1,4-dioxa-7-azaspiro[4.4]nonane-8-carboxylate (90 mg, 0.18 mmol) in MeOH (0.9 mL) and water (0.3 mL) was added LiOH·H₂O (23 mg, 0.54 mmol) at 0° C. and the mixture was stirred at room temperature for 16 hours. The mixture was acidified with 1 N aq·HCl solution to pH ˜3 and extracted with ethyl acetate. The combined organic layers were concentrated to dryness under reduced pressure to give (S)-7-((5,5-dioxidodibenzo[b,d]thiophene-2-carbonyl)glycyl)-1,4-dioxa-7-azaspiro[4.4]nonane-8-carboxylic acid (76 mg, yield 86.9%) as a white solid, which was used directly in next step without purification. LC/MS (ESI) (m/z): 473(M+H)⁺.

### Step 3: (S)—N—((R)-1-(4-carbamimidoylthiophen-2-yl)ethyl)-7-((5,5-dioxidodibenzo[b,d]thiophene-2-carbonyl)glycyl)-1,4-dioxa-7-azaspiro[4.4]nonane-8-carboxamide (Compound 261)

To a solution of (S)-7-((5,5-dioxidodibenzo[b,d]thiophene-2-carbonyl)glycyl)-1,4-dioxa-7-azaspiro[4.4]nonane-8-carboxylic acid (35 mg, 0.07 mmol) and (R)-5-(1-aminoethyl)thiophene-3-carboximidamide hydrochloride (29 mg, 0.14 mmol) in DMF (0.5 mL) was added DIPEA (39 mg, 0.3 mmol) and PyBOP (36 mg, 0.07 mmol) at 0° C. under N₂ atmosphere, the mixture was stirred at 25° C. for 1 hour. The reaction mixture was diluted with water and extracted with EtOAc twice. The combined organic layers were washed with brine, dried over anhydrous Na₂SO₄, filtered, and concentrated to dryness under reduced pressure. The residue was purified by prep-HPLC to give Compound 261 (1.5 mg, yield 3.3%) as a white solid. ¹H NMR (400 MHz, CD₃OD) δ 8.50 (s, 1H), 8.23 (d, J=14.0 Hz, 1H), 8.07 (t, J=7.4 Hz, 2H), 7.95 (d, J=8.0 Hz, 1H), 7.89 (d, J=7.5 Hz, 1H), 7.80 (t, J=7.5 Hz, 1H), 7.68 (t, J=7.8 Hz, 1H), 7.52 (s, 1H), 5.29 (d, J=6.9 Hz, 1H), 4.57 (d, J=8.1 Hz, 1H), 4.24 (q, J=16.9 Hz, 2H), 4.05-3.97 (m, 4H), 3.81 (q, J=10.6 Hz, 2H), 2.50-2.39 (m, 1H), 2.22 (dd, J=13.3, 6.7 Hz, 1H), 1.59 (d, J=7.0 Hz, 3H). LCMS (ESI) (m/z): 624 (M+H)⁺.

### Scheme 252: Synthesis of (2S,4R)—N—((R)-1-(4-carbamimidoylthiophen-2-yl)ethyl)-1-((9,9-difluoro-9H-fluorene-2-carbonyl)glycyl)-4-(difluoromethoxy)pyrrolidine-2-carboxamide (Compound 262)

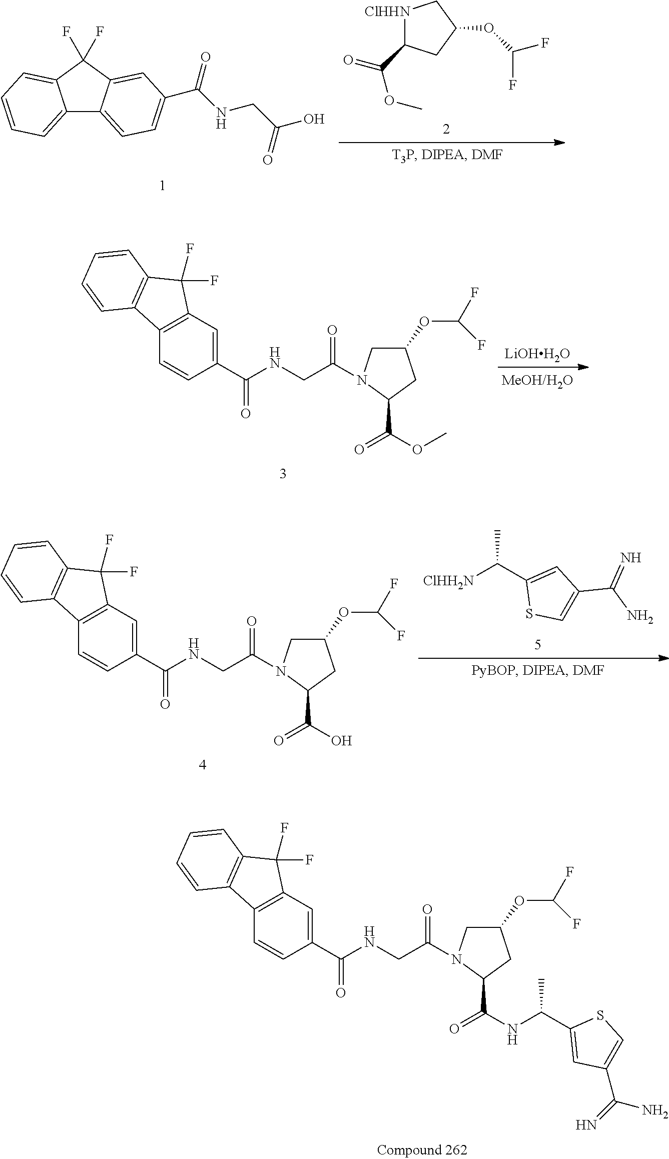

Compound 262 was prepared from (9,9-difluoro-9H-fluorene-2-carbonyl)glycine, methyl (2S,4R)-4-(difluoromethoxy)pyrrolidine-2-carboxylate hydrochloride, and (R)-5-(1-aminoethyl)thiophene-3-carboximidamide hydrochloride based on the procedures set forth in Scheme 3. ¹H NMR (400 MHz, CD₃OD) δ 8.13 (s, 1H), 8.06 (d, J=8.0 Hz, 2H), 7.83-7.79 (m, 2H), 7.66 (d, J=7.5 Hz, 1H), 7.57 (d, J=7.5 Hz, 1H), 7.48 (d, J=11.1 Hz, 2H), 6.51 (dd, J=74.2, 57.8 Hz, 1H), 5.27 (q, J=6.9 Hz, 1H), 5.03 (s, 1H), 4.59 (t, J=7.8 Hz, 1H), 4.30 (d, J=16.7 Hz, 1H), 4.17 (d, J=16.5 Hz, 1H), 3.99-3.94 (m, 1H), 3.88 (d, J=12.4 Hz, 1H), 2.52-2.45 (m, 1H), 2.25 (dd, J=13.3, 5.6 Hz, 1H), 1.58 (d, J=6.9 Hz, 3H). LCMS (ESI) (m/z): 618 (M+H)⁺.

### Scheme 253: Synthesis of (2S,4R)—N—((R)-1-(4-carbamimidoylthiophen-2-yl)ethyl)-1-((9,9-difluoro-9H-fluorene-2-carbonyl)glycyl)-4-fluoro-4-(fluoromethyl)pyrrolidine-2-carboxamide (Compound 263)

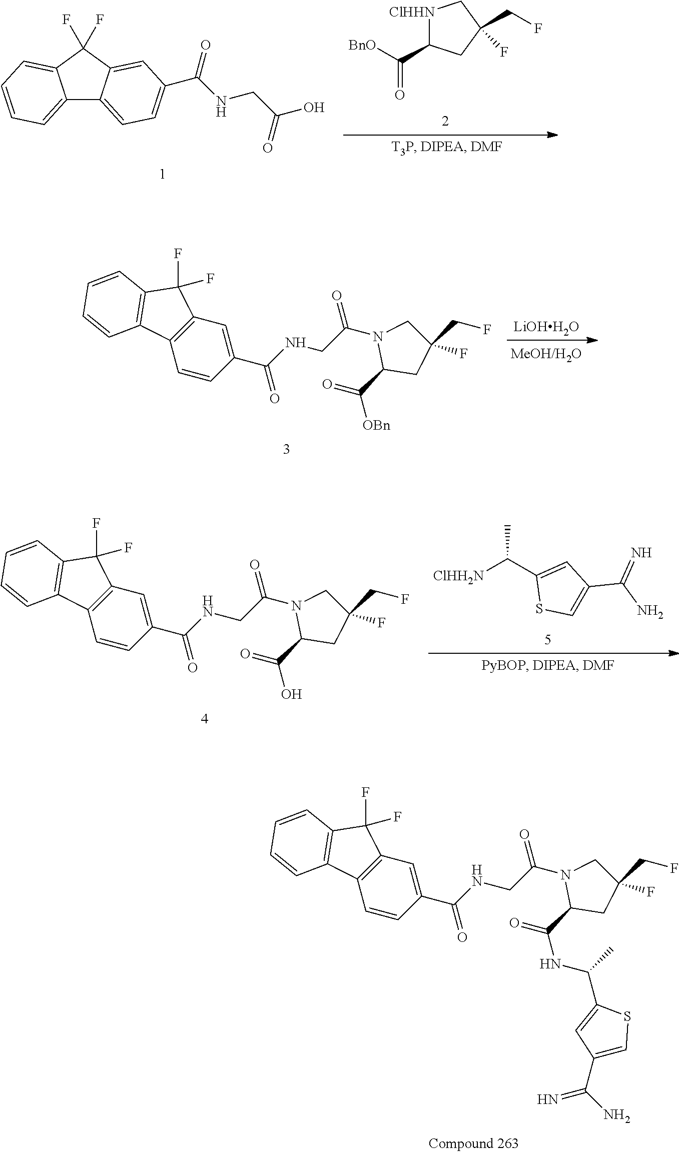

Compound 263 was prepared from (9,9-difluoro-9H-fluorene-2-carbonyl)glycine, benzyl (2S,4R)-4-fluoro-4-(fluoromethyl)pyrrolidine-2-carboxylate hydrochloride, and (R)-5-(1-aminoethyl)thiophene-3-carboximidamide hydrochloride based on the procedures set forth in Scheme 3. ¹H NMR (400 MHz, CD₃OD) δ 8.12 (s, 1H), 8.10-7.92 (m, 2H), 7.83-7.77 (m, 2H), 7.66 (d, J=7.6 Hz, 1H), 7.58 (t, J=7.3 Hz, 1H), 7.47 (dd, J=14.8, 7.3 Hz, 2H), 5.26 (d, J=6.9 Hz, 1H), 4.75 (t, J=10.1 Hz, 1H), 4.66 (dd, J=13.7, 5.2 Hz, 2H), 4.34 (d, J=16.7 Hz, 1H), 4.21-4.06 (m, 2H), 3.96 (dd, J=32.1, 12.2 Hz, 1H), 2.70-2.51 (m, 1H), 2.33-2.12 (m, 1H), 1.57 (d, J=6.9 Hz, 3H). LCMS (ESI) (m/z): 602 (M+H)⁺.

### Scheme 254: Synthesis of (2S,4R)—N—((R)-1-(4-carbamimidoylthiophen-2-yl)ethyl)-1-((9,9-difluoro-9H-fluorene-2-carbonyl)glycyl)-4-fluoro-4-(methoxymethyl)pyrrolidine-2-carboxamide (Compound 264)

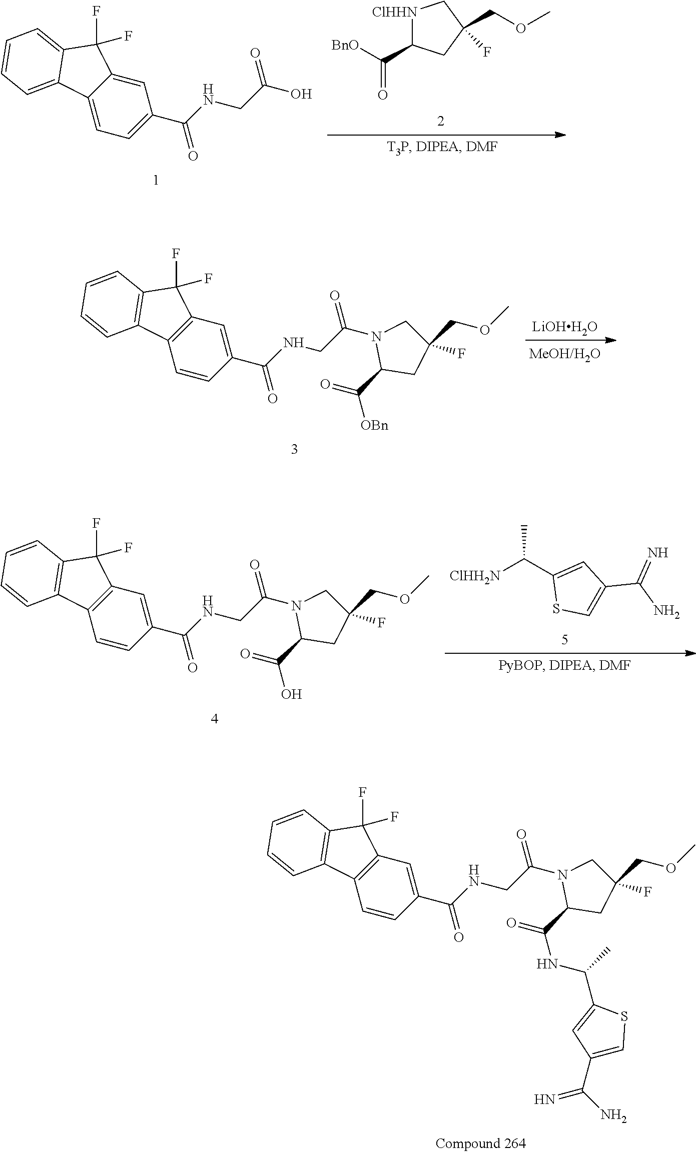

Compound 264 was prepared from (9,9-difluoro-9H-fluorene-2-carbonyl)glycine, benzyl (2S,4R)-4-fluoro-4-(methoxymethyl)pyrrolidine-2-carboxylate hydrochloride, and (R)-5-(1-aminoethyl)thiophene-3-carboximidamide hydrochloride based on the procedures set forth in Scheme 3. ¹H NMR (400 MHz, CD₃OD) δ 8.21 (s, 1H), 8.14-8.01 (m, 2H), 7.86-7.76 (m, 2H), 7.65 (s, 1H), 7.56 (d, J=16.6 Hz, 2H), 7.47 (s, 1H), 5.34 (s, 1H), 4.32 (d, J=16.7 Hz, 1H), 4.15 (d, J=16.4 Hz, 1H), 3.88 (s, 1H), 3.74-3.66 (m, 2H), 2.22-2.16 (m, 2H), 2.03 (s, 2H), 1.58 (d, J=6.9 Hz, 3H), 0.90 (s, 3H). LCMS (ESI) (m/z): 614 (M+H)⁺.

### Scheme 255: Synthesis of (2S,4R)—N—((R)-1-(4-carbamimidoylthiophen-2-yl)ethyl)-1-((9,9-difluoro-9H-fluorene-2-carbonyl)glycyl)-4-(methylsulfonyl)pyrrolidine-2-carboxamide (Compound 265)

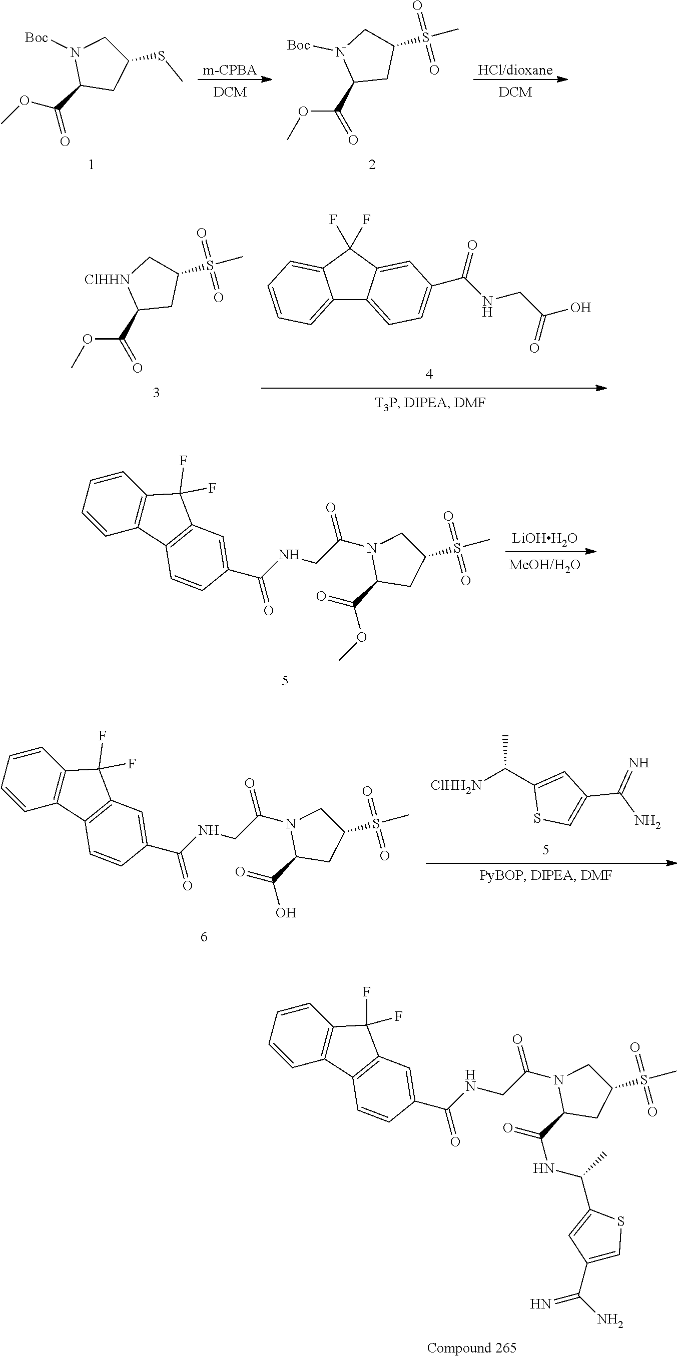

### Step 1: 1-(tert-butyl) 2-methyl (2S,4R)-4-(methylsulfonyl)pyrrolidine-1,2-dicarboxylate (2)

To a mixture of 1-(tert-butyl) 2-methyl (2S,4R)-4-(methylthio)pyrrolidine-1,2-dicarboxylate (100 mg, 0.36 mmol) in DCM (0.5 mL) was added m-CPBA (125 mg, 0.72 mmol) under N₂ atmosphere, the mixture was stirred at room temperature for 4 hours. The mixture was concentrated under reduced pressure to dryness. The residue was purified by flash chromatography (silica gel, 0-70% EtOAc in PE) to give 1-(tert-butyl) 2-methyl (2S,4R)-4-(methylsulfonyl)pyrrolidine-1,2-dicarboxylate (95 mg, yield 85.1%) as a yellow solid. LC/MS (ESI) (m/z): 308 (M+H)⁺.

### Step 2: methyl (2S,4R)-4-(methylsulfonyl)pyrrolidine-2-carboxylate hydrochloride (3)

To a mixture of 1-(tert-butyl) 2-methyl (2S,4R)-4-(methylsulfonyl)pyrrolidine-1,2-dicarboxylate (95 mg, 0.31 mmol) in DCM (1 mL) was added HCl/1,4-dioxane (0.8 mL, 3.2 mmol) at 0° C., and the mixture was stirred at room temperature for 2 hours. The mixture was concentrated to dryness under reduced pressure to give methyl (2S,4R)-4-(methylsulfonyl)pyrrolidine-2-carboxylate hydrochloride (75 mg, yield 98.9%) as a yellow solid, which was used in next step without purification. LC/MS (ESI) m/z: 208 (M+H)⁺.

### Step 3: methyl (2S,4R)-1-((9,9-difluoro-9H-fluorene-2-carbonyl)glycyl)-4-(methylsulfonyl)pyrrolidine-2-carboxylate (5)

To a mixture of (9,9-difluoro-9H-fluorene-2-carbonyl)glycine (80 mg, 0.26 mmol) and methyl (2S,4R)-4-(methylsulfonyl)pyrrolidine-2-carboxylate hydrochloride (76 mg, 0.31 mmol) in DMF (1 mL) was added DIPEA (101 mg, 0.78 mmol) and T₃P (248 mg, 0.39 mmol, 50% wt. in EtOAc) under N₂ atmosphere, the mixture was stirred at room temperature for 1 hour. The mixture was diluted with EtOAc, washed with saturated aq·Na₂CO₃ solution and brine, dried over anhydrous Na₂SO₄, filtered, and concentrated under reduced pressure to dryness. The residue was purified by flash chromatography (silica gel, 0-85% EtOAc in PE) to give methyl (2S,4R)-1-((9,9-difluoro-9H-fluorene-2-carbonyl)glycyl)-4-(methylsulfonyl)pyrrolidine-2-carboxylate (92 mg, yield 71.9%) as a yellow solid. LC/MS (ESI) (m/z): 493 (M+H)⁺.

### Step 4: (2S,4R)-1-((9,9-difluoro-9H-fluorene-2-carbonyl)glycyl)-4-(methylsulfonyl) pyrrolidine-2-carboxylic acid (6)

To a mixture of methyl (2S,4R)-1-((9,9-difluoro-9H-fluorene-2-carbonyl)glycyl)-4-(methylsulfonyl)pyrrolidine-2-carboxylate (80 mg, 0.16 mmol) in MeOH (0.9 mL) and water (0.3 mL) was added LiOH·H₂O (20 mg, 0.48 mmol) at 0° C., and the mixture was stirred at room temperature overnight. The mixture was acidified with 1 N aq·HCl to pH=3 and extracted with EtOAc twice. The combined organic layers were washed with brine, dried over anhydrous Na₂SO₄, filtered, and concentrated to dryness under reduced pressure to give (2S,4R)-1-((9,9-difluoro-9H-fluorene-2-carbonyl)glycyl)-4-(methylsulfonyl)pyrrolidine-2-carboxylic acid (50 mg, yield 65.3%) as a yellow solid, which was used in next step without purification LC/MS (ESI) m/z: 479 (M+H)⁺.

### Step 5: (2S,4R)—N—((R)-1-(4-carbamimidoylthiophen-2-yl)ethyl)-1-((9,9-difluoro-9H-fluorene-2-carbonyl)glycyl)-4-(methylsulfonyl)pyrrolidine-2-carboxamide (Compound 265)

To a solution of (2S,4R)-1-((9,9-difluoro-9H-fluorene-2-carbonyl)glycyl)-4-(methylsulfonyl)pyrrolidine-2-carboxylic acid (40 mg, 0.08 mmol) and (R)-5-(1-aminoethyl)thiophene-3-carboximidamide hydrochloride (29 mg, 0.14 mmol) in DMF (0.4 mL) was added DIPEA (39 mg, 0.3 mmol) and PyBOP (41 mg, 0.08 mmol) at 0° C. under N₂ atmosphere, the mixture was stirred at 25° C. for 1 hour. The reaction mixture was diluted with water and extracted with EtOAc twice. The combined organic layers were washed with brine, dried over anhydrous Na₂SO₄, filtered, and concentrated to dryness under reduced pressure. The residue was purified by prep-HPLC to give Compound 265 (2.9 mg, yield 5.8%) as a white solid. ¹H NMR (400 MHz, CD₃OD) δ 8.22 (s, 1H), 8.13 (s, 1H), 8.07 (d, J=6.8 Hz, 1H), 7.82 (s, 2H), 7.66 (s, 1H), 7.59 (s, 1H), 7.50 (s, 2H), 5.28 (d, J=7.3 Hz, 1H), 4.73-4.70 (m, 1H), 4.30 (s, 1H), 4.16 (s, 4H), 3.48 (s, 1H), 3.09 (s, 3H), 3.04 (s, 1H), 2.45 (s, 1H), 1.60 (d, J=7.1 Hz, 3H). LCMS (ESI) (m/z): 630 (M+H)⁺.

### Scheme 256: Synthesis of (S)—N-((4-carbamimidoylthiophen-2-yl)methyl)-7-((4-(3-fluoropyridin-4-yl)benzoyl)glycyl)-1,4-dioxa-7-azaspiro[4.4]nonane-8-carboxamide (Compound 266)

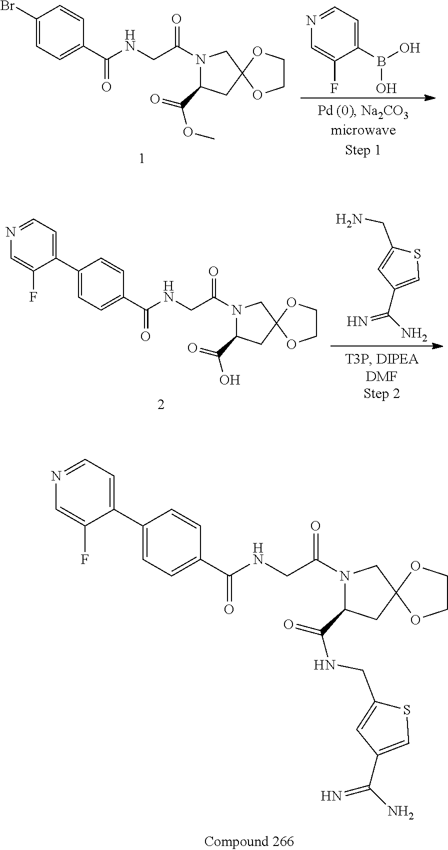

Compound 266 was prepare from (3-fluoro-4-pyridyl)boronic acid, methyl (8S)-7-[2-[(4-bromobenzoyl)amino]acetyl]-1,4-dioxa-7-azaspiro[4.4]nonane-8-carboxylate, and 5-(aminomethyl)thiophene-3-carboximidamide. HCl based on the procedures set forth in Scheme 206. LC/MS (ESI) m/z: 567 (M+H)⁺. ¹H NMR (400 MHz, DMSO) δ 8.84 (s, 1H), 8.69 (d, J=2.4 Hz, 1H), 8.65 (s, 1H), 8.54 (d, J=4.6 Hz, 1H), 8.42 (s, 1H), 8.02 (d, J=7.8 Hz, 2H), 7.79 (d, J=7.8 Hz, 2H), 7.70 (s, 1H), 7.10 (s, 1H), 6.73 (s, 1H), 4.55 (s, 1H), 4.48 (d, J=5.7 Hz, 2H), 4.41 (q, J=7.6 Hz, 1H), 4.21 (dd, J=16.6, 5.5 Hz, 1H), 3.99-3.87 (m, 5H), 3.81 (d, J=10.7 Hz, 1H), 3.74-3.60 (m, 1H), 2.37-2.27 (m, 1H), 2.06 (dd, J=13.2, 6.7 Hz, 1H), 1.49-1.43 (m, 1H).

### Scheme 257: Synthesis of (S)—N-((4-carbamimidoylthiophen-2-yl)methyl)-7-(2-(5-(2,4-difluorophenyl) isoindolin-2-yl)acetyl)-1,4-dioxa-7-azaspiro[4.4]nonane-8-carboxamide (Compound 287)

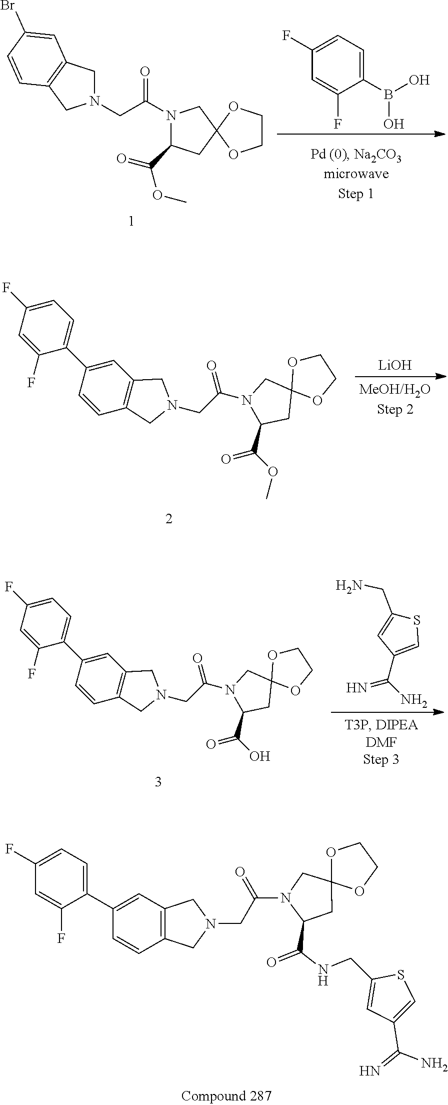

Compound 287 was prepared from (2,4-difluorophenyl)boronic acid, methyl (8S)-7-[2-(5-bromoisoindolin-2-yl)acetyl]-1,4-dioxa-7-azaspiro[4.4]nonane-8-carboxylate, and 5-(aminomethyl)thiophene-3-carboximidamide HCl based on the procedures set forth in Scheme 199. LC/MS (ESI) m/z: 582 (M+H)⁺. ¹H NMR (400 MHz, DMSO) δ 8.69 (t, J=6.0 Hz, 1H), 8.36 (s, 1H), 7.80 (s, 1H), 7.55 (td, J=8.9, 6.6 Hz, 1H), 7.34 (td, J=10.4, 3.4 Hz, 2H), 7.22-7.10 (m, 2H), 4.59-4.33 (m, 3H), 4.10-3.91 (m, 4H), 3.89 (td, J=10.5, 6.2 Hz, 3H), 3.81-3.67 (m, 1H), 3.62-3.48 (m, 3H), 3.03 (p, J=6.5 Hz, 1H), 2.50-2.39 (m, 1H), 2.29 (dd, J=13.3, 8.8 Hz, 1H), 2.02 (dd, J=13.1, 7.2 Hz, 1H), 1.47 (s, 1H), 0.98 (d, J=6.5 Hz, 2H).

### Scheme 258: Synthesis of (S)—N-((4-carbamimidoylthiophen-2-yl)methyl)-7-((2′,4′-difluoro-[1,1′-biphenyl]-4-carbonyl)-L-alanyl)-1,4-dioxa-7-azaspiro[4.4]nonane-8-carboxamide (Compound 291)

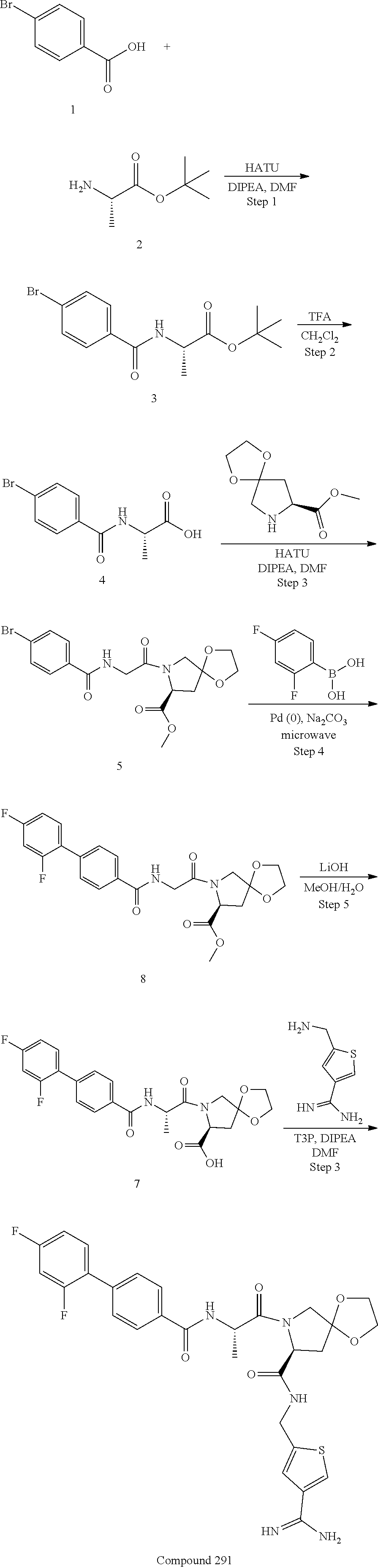

### Step 1: tert-butyl (4-bromobenzoyl)-L-alaninate (3)

To a solution of 4-bromobenzoic acid (1.000 g, 4.975 mmol, 1 equiv.) in DMF (10 mL) was added H-ALA-OTBU HCL (0.9170 g, 5.970 mmol, 1.2 equiv.), HATU (2.459 g, 6.467 mmol, 1.3 equiv.) and DIPEA (2.600 mL, 14.9 mmol, 3 equiv.). The reaction was stirred at room temperature for 1 h. Water (10 mL) was added, and the reaction stirred for 10 minutes. The product crashed out and the solid was collected by filtration and dried to give tert-butyl (4-bromobenzoyl)-L-alaninate (1.514 g, 4.613 mmol, 92.73% yield) as a white solid. LC/MS (ESI) m/z: 328/330 (M+H)⁺.

### Step 2: (4-bromobenzoyl)-L-alanine (4)

TFA (3.456 mL) was added to a solution of tert-butyl (4-bromobenzoyl)-L-alaninate (1.500 g, 4.570 mmol, 1.0 equiv.) in CH₂Cl₂ (10 mL) at ice-bath temperature. The reaction was stirred at room temperature for 3 hours and then concentrated to dryness to give (2S)-2-[(4-bromobenzoyl)amino]propanoic acid (1.000 g, 3.675 mmol, 80.41% yield) as a pale-yellow solid which was used in the next step without further purification. LC/MS (ESI) m/z: 272 (M+H)⁺.

### Step 3: methyl (S)-7-((4-bromobenzoyl)-L-alanyl)-1,4-dioxa-7-azaspiro[4.4]nonane-8-carboxylate (5)

To a solution of (2S)-2-[(4-bromobenzoyl)amino]propanoic acid (0.3 g, 1.103 mmol, 1.0 equiv.) in DMF (5 mL) was added methyl (8S)-1,4-dioxa-7-azaspiro[4.4]nonane-8-carboxylate (0.2477 g, 1.323 mmol, 1.2 equiv.), HATU (0.545 g, 1.433 mmol, 1.3 equiv.) and DIPEA (0.5770 mL, 3.31 mmol, 3.0 equiv.). The reaction was stirred at room temperature for 30 minutes. Water (5 mL) and EtOAc (5 mL) were added, and the two layers separated. The aq. layer was extracted twice with EtOAc, washed with brine, dried over Na₂SO₄, and concentrated. Purification by Combi Flash, 12 g column, solvent A=CH₂Cl₂, solvent B=MeOH, 100% A to 5% B gave methyl (8S)-7-[(2S)-2-[(4-bromobenzoyl)amino]propanoyl]-1,4-dioxa-7-azaspiro[4.4]nonane-8-carboxylate (0.3721 g, 0.8432 mmol, 76.48% yield) as a white solid. LC/MS (ESI) m/z: 442 (M+H)⁺.

### Step 4: methyl (S)-7-((2′,4′-difluoro-[1,1′-biphenyl]-4-carbonyl)-L-alanyl)-1,4-dioxa-7-azaspiro[4.4]nonane-6-carboxylate (6)

A mixture of (2,4-difluorophenyl)boronic acid (19.32 mg, 0.1223 mmol, 1.2 equiv.), methyl (8S)-7-[(2S)-2-[(4-bromobenzoyl)amino]propanoyl]-1,4-dioxa-7-azaspiro[4.4]nonane-8-carboxylate (45 mg, 0.1020 mmol, 1.0 equiv.), Pd(dppf)Cl₂·CH₂Cl₂ (8.410 mg, 0.0102 mmol, 0.1 equiv.), and Na₂CO₃ (21.62 mg, 0.2040 mmol, 2 equiv.) in 1,4-dioxane (3 mL) and water (0.3 mL) was stirred in a microwave reactor at 110° C. for 30 min. The reaction was cooled to room temperature and then filtered through Celite pad. The filtrate extracted with EtOAc, washed with brine, dried over Na₂SO₄ and concentrated. Purification by combi-Flash; 4 g column, solvent A=CH₂Cl₂, solvent B=MeOH; 100% A to 5% B gave methyl (8S)-7-[(2S)-2-[[4-(2,4-difluorophenyl)benzoyl]amino]propanoyl]-1,4-dioxa-7-azaspiro[4.4]nonane-8-carboxylate (34.00 mg, 0.07167 mmol, 70.28% yield). LC/MS (ESI) m/z: 475 (M+H)⁺.

### Step 5: methyl (S)-7-((2′,4′-difluoro-[1,1′-biphenyl]-4-carbonyl)-L-alanyl)-1,4-dioxa-7-azaspiro[4.4]nonane-8-carboxylate (7)

To a solution of methyl (8S)-7-[(2S)-2-[[4-(2,4-difluorophenyl)benzoyl]amino]propanoyl]-1,4-dioxa-7-azaspiro[4.4]nonane-8-carboxylate (30.00 mg, 0.06324 mmol, 1.00 equiv.) in MeOH (1.5 mL) water (0.5 mL) was added LiOH (1.817 mg, 0.07587 mmol, 1.2 equiv.). The reaction was stirred at room temperature overnight. The reaction was then concentrated to remove MeOH and diluted with EtOAc. The pH was adjusted to 1 using 1 N HCl. The two layers were separated, and the aq. layer was extracted with EtOAc twice. The combined organic extracts were washed with brine, dried over Na₂SO₄, and concentrated to give (8S)-7-[(2S)-2-[[4-(2,4-difluorophenyl)benzoyl]amino]propanoyl]-1,4-dioxa-7-azaspiro[4.4]nonane-8-carboxylic acid (26.17 mg, 0.05684 mmol, 89.89% yield) as a white solid. LC/MS (ESI) m/z: 461 (M+H)⁺.

### Step 6: (S)—N-((4-carbamimidoylthiophen-2-yl)methyl)-7-((2′,4′-difluoro-[1,1′-biphenyl]-4-carbonyl)-L-alanyl)-1,4-dioxa-7-azaspiro[4.4]nonane-8-carboxamide (Compound 291)

To a solution of (8S)-7-[(2S)-2-[[4-(2,4-difluorophenyl)benzoyl]amino]propanoyl]-1,4-dioxa-7-azaspiro[4.4]nonane-8-carboxylic acid (25.00 mg, 0.05430 mmol, 1.0 equiv.) in DMF (1 mL) was added 5-(aminomethyl)thiophene-3-carboximidamide. HCl (12.49 mg, 0.06516 mmol, 1.2 equiv.), T3P, (50 mass %) in ehthyl acetate (48.80 μL, 0.0815 mmol, 1.5 equiv.), and DIPEA (28.40 μL, 0.163 mmol, 3.0 equiv.). The reaction mixture was stirred at room temperature for 30 minutes and then purified directly by HPLC to give Compound 291 (7.000 mg, 0.01171 mmol, 21.57% yield) LC/MS (ESI) m/z: 598 (M+H)⁺. ¹H NMR (400 MHz, DMSO) δ 8.87 (s, 1H), 8.63 (d, J=7.1 Hz, 1H), 8.57 (s, 1H), 8.36 (d, J=2.0 Hz, 1H), 8.27 (s, 1H), 7.91 (d, J=8.0 Hz, 1H), 7.81 (d, J=7.6 Hz, 1H), 7.57 (t, J=8.3 Hz, 1H), 7.37-7.27 (m, 1H), 7.16 (d, J=17.8 Hz, 1H), 7.16 (s, 1H), 7.04 (s, 1H), 6.94 (t, J=9.2 Hz, 1H), 4.66 (t, J=7.1 Hz, 2H), 4.44-4.37 (m, 1H), 4.32 (t, J=8.2 Hz, 2H), 3.88 (d, J=11.2 Hz, 1H), 3.73-3.62 (m, 3H), 3.54 (d, J=10.8 Hz, 2H), 2.31 (d, J=12.3 Hz, 1H), 2.22 (d, J=10.0 Hz, 2H), 2.11-2.03 (m, 1H), 1.95 (d, J=12.5 Hz, 2H), 1.26 (d, J=6.9 Hz, 3H).

### Scheme 259: Synthesis of (S)—N-((4-carbamimidoylthiophen-2-yl)methyl)-7-((4′-fluoro-[1,1′-biphenyl]-4-carbonyl)-L-alanyl)-1,4-dioxa-7-azaspiro[4.4]nonane-8-carboxamide (Compound 296)

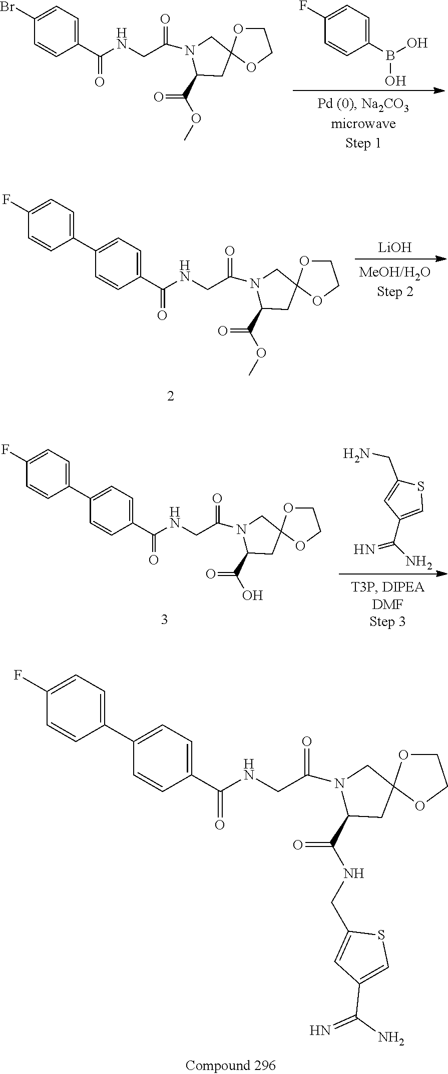

Compound 296 was prepared from (4-fluorophenyl)boronic acid, methyl (8S)-7-[(2S)-2-[(4-bromobenzoyl)amino]propanoyl]-1,4-dioxa-7-azaspiro[4.4]nonane-8-carboxylate, and 5-(aminomethyl)thiophene-3-carboximidamide HCl based on the procedures set forth in Scheme 199. LC/MS (ESI) m/z: 580 (M+H)⁺. ¹H NMR (400 MHz, DMSO) δ 8.80-8.56 (m, 1H), 8.42 (s, 1H), 7.97 (d, J=8.1 Hz, 1H), 7.89-7.60 (m, 3H), 7.32 (t, J=8.7 Hz, 1H), 7.11 (d, J=11.3 Hz, 1H), 4.72 (t, J=7.1 Hz, 1H), 4.60-4.45 (m, 1H), 4.39 (t, J=8.1 Hz, 1H), 4.05-3.81 (m, 4H), 3.81-3.65 (m, 2H), 2.98 (p, J=6.7 Hz, 2H), 2.50 (p, J=1.9 Hz, 3H), 2.37-2.23 (m, 1H), 2.03 (dd, J=13.1, 7.7 Hz, 3H), 1.33 (d, J=7.0 Hz, 2H).

### Scheme 260: Synthesis of (S)—N-((4-carbamimidoylthiophen-2-yl)methyl)-7-((5-phenylfuran-2-carbonyl)glycyl)-1,4-dioxa-7-azaspiro[4.4]nonane-8-carboxamide (Compound 297)

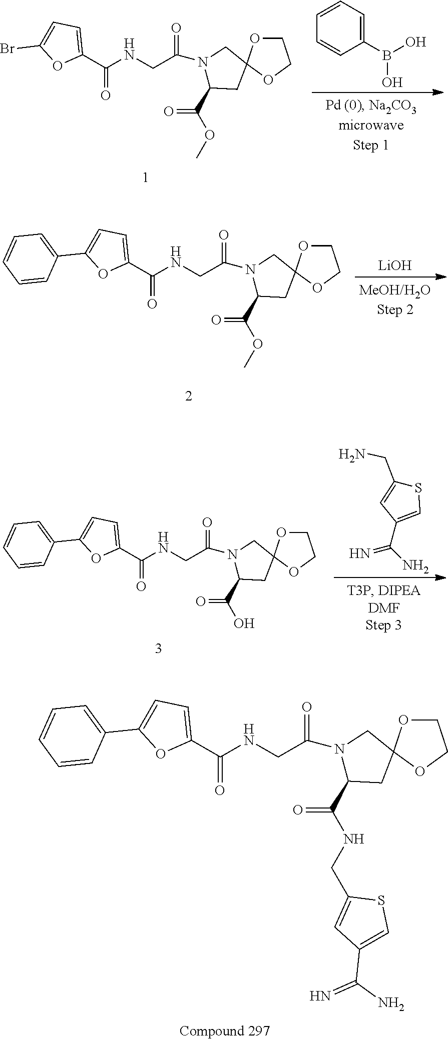

Compound 297 was prepared from phenylboronic acid, methyl (8S)-7-[2-[(5-bromofuran-2-carbonyl)amino]acetyl]-1,4-dioxa-7-azaspiro[4.4]nonane-8-carboxylate, and 5-(aminomethyl)thiophene-3-carboximidamide HCl based on the procedures set forth in Scheme 199. LC/MS (ESI) m/z: 538 (M+H)⁺. ¹H NMR (400 MHz, DMSO) δ 8.64 (s, 1H), 8.32 (s, 1H), 7.55-6.63 (m, 6H), 4.82-4.30 (m, 4H), 4.17 (d, J=15.6 Hz, 2H), 3.76-3.40 (m, 4H), 3.23-2.99 (m, 3H), 2.32-2.07 (m, 2H), 1.48-1.33 (m, 2H).

### Scheme 261: Synthesis of (S)—N-((4-carbamimidoylthiophen-2-yl)methyl)-7-((5-(2,4-difluorophenyl)furan-2-carbonyl)glycyl)-1,4-dioxa-7-azaspiro[4.4]nonane-8-carboxamide (Compound 298)

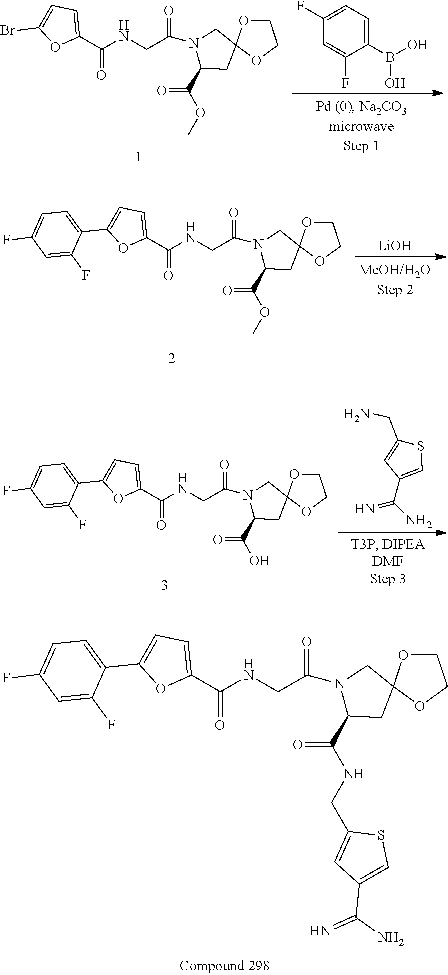

Compound 298 was prepared from (2,4-difluorophenyl)boronic acid, methyl (8S)-7-[2-[(5-bromofuran-2-carbonyl)amino]acetyl]-1,4-dioxa-7-azaspiro[4.4]nonane-8-carboxylate, and 5-(aminomethyl)thiophene-3-carboximidamide HCl based on the procedures set forth in Scheme 199. LC/MS (ESI) m/z: 574 (M+H)⁺. ¹H NMR (400 MHz, DMSO) δ 9.02 (s, 1H), 8.75 (s, 1H), 8.66 (s, 1H), 8.41 (s, 1H), 8.17 (d, J=8.0 Hz, 2H), 7.77 (s, 1H), 7.45 (t, J=10.4 Hz, 1H), 7.37-7.17 (m, 2H), 7.12 (s, 1H), 6.94 (s, 1H), 4.58-4.28 (m, 3H), 4.18 (d, J=16.5 Hz, 1H), 4.08-3.85 (m, 4H), 3.81 (d, J=10.8 Hz, 1H), 3.11-2.83 (m, 2H), 2.50 (p, J=1.8 Hz, 2H), 2.32-2.06 (m, 2H).

### Scheme 262: Synthesis of (S)—N-((4-carbamimidoylthiophen-2-yl)methyl)-7-((5-(4-fluorophenyl)furan-2-carbonyl)glycyl)-1,4-dioxa-7-azaspiro[4.4]nonane-8-carboxamide (Compound 299)

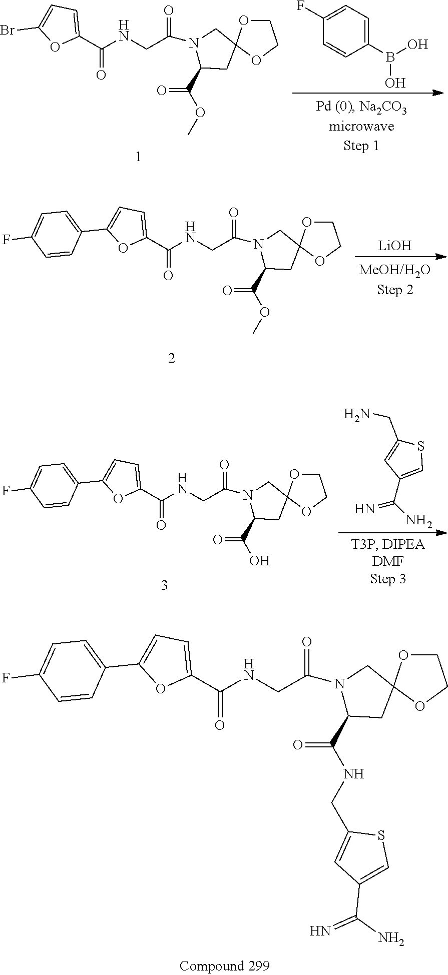

Compound 299 was prepared from (4-fluorophenyl)boronic acid, methyl (8S)-7-[2-[(5-bromofuran-2-carbonyl)amino]acetyl]-1,4-dioxa-7-azaspiro[4.4]nonane-8-carboxylate, and 5-(aminomethyl)thiophene-3-carboximidamide HCl based on the procedures set forth in Scheme 199. LC/MS (ESI) m/z: 556 (M+H)⁺. ¹H NMR (400 MHz, DMSO) δ 8.65 (d, J=7.4 Hz, 1H), 8.43 (s, 1H), 7.97 (dd, J=8.5, 5.3 Hz, 1H), 7.87-7.62 (m, 1H), 7.33 (t, J=8.7 Hz, 1H), 7.20 (d, J=3.6 Hz, 1H), 7.09 (d, J=3.6 Hz, 1H), 4.71 (s, 1H), 4.55 (d, J=5.4 Hz, 1H), 4.47 (d, J=5.8 Hz, 2H), 4.39 (t, J=7.8 Hz, 1H), 4.17 (dd, J=16.8, 5.3 Hz, 2H), 4.04-3.83 (m, 4H), 3.80 (d, J=10.9 Hz, 1H), 3.62 (d, J=10.8 Hz, 1H), 2.32 (dd, J=13.3, 8.6 Hz, 1H), 2.05 (dd, J=13.2, 6.9 Hz, 1H).

### Scheme 263: Synthesis of (S)-7-(([1,1′-biphenyl]-4-carbonyl)-L-alanyl)-N-((4-carbamimidoylthiophen-2-yl)methyl)-1,4-dioxa-7-azaspiro[4.4]nonane-8-carboxamide (Compound 300)

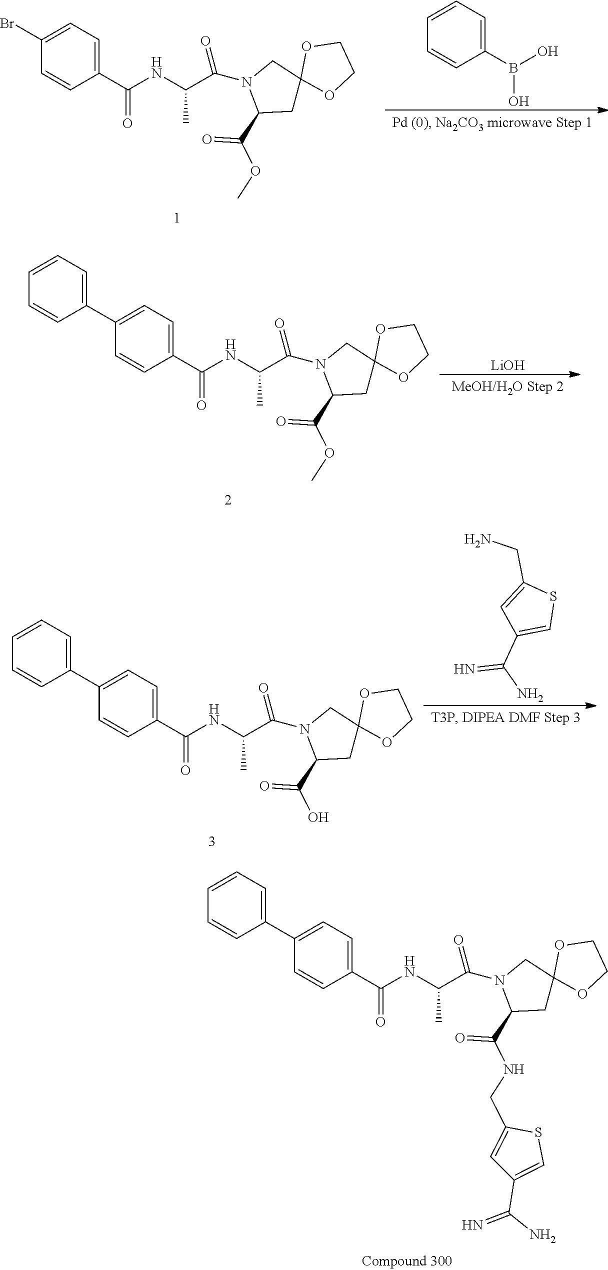

Compound 300 was prepared from phenylboronic acid, methyl (8S)-7-[(2S)-2-[(4-bromobenzoyl)amino]propanoyl]-1,4-dioxa-7-azaspiro[4.4]nonane-8-carboxylate, and 5-(aminomethyl)thiophene-3-carboximidamide HCl based on the procedures set forth in Scheme 199. LC/MS (ESI) m/z: 562 (M+H)⁺. ¹H NMR (400 MHz, DMSO) δ 8.76-8.53 (m, 1H), 8.38 (s, 1H), 8.35 (s, 1H), 7.98 (d, J=8.0 Hz, 1H), 7.93-7.78 (m, 1H), 7.50 (t, J=7.5 Hz, 2H), 7.41 (t, J=7.3 Hz, 2H), 7.12 (s, 1H), 4.72 (t, J=7.1 Hz, 2H), 4.63-4.25 (m, 2H), 3.99-3.78 (m, 4H), 3.75 (d, J=4.6 Hz, 2H), 2.32 (q, J=11.2 Hz, 1H), 2.22-1.95 (m, 2H), 1.34 (d, J=6.9 Hz, 3H), 0.90 (d, J=7.3 Hz, 2H).

### Scheme 264: Synthesis of (S)—N-((4-carbamimidoylthiophen-2-yl)methyl)-7-((2′,4′-difluoro-[1,1′-biphenyl]-4-carbonyl)-D-alanyl)-1,4-dioxa-7-azaspiro[4.4]nonane-8-carboxamide (Compound 301)

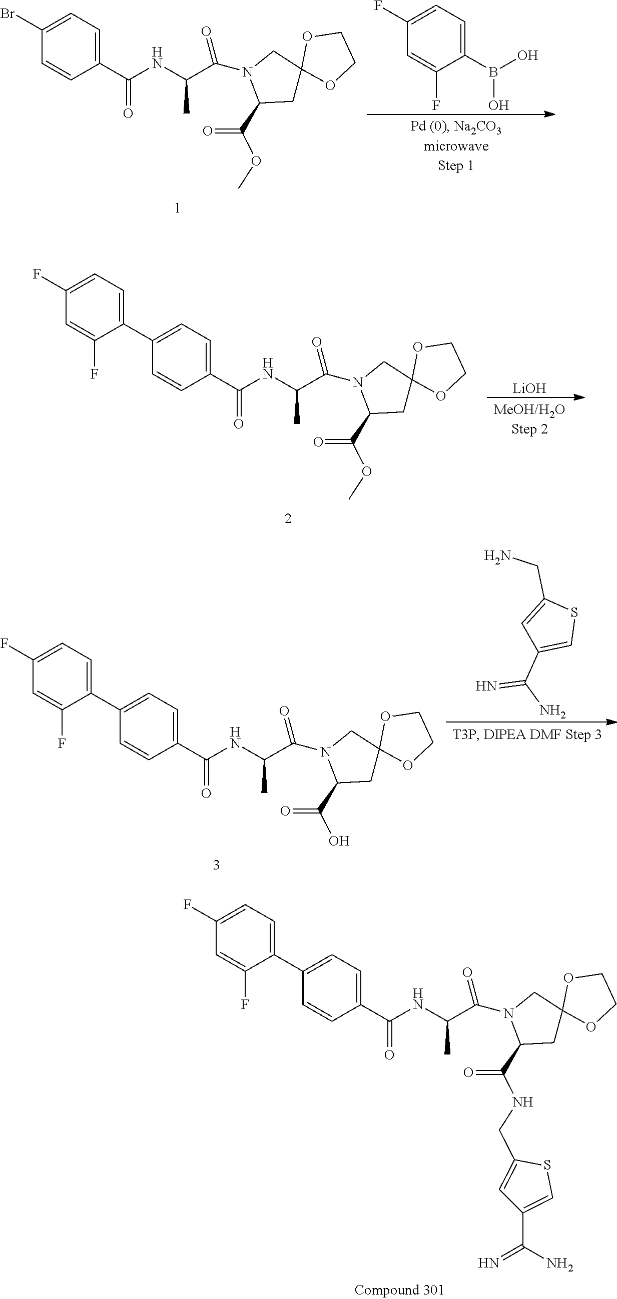

Compound 301 was prepared from (2,4-difluorophenyl)boronic acid, methyl (8S)-7-[(2R)-2-[(4-bromobenzoyl)amino]propanoyl]-1,4-dioxa-7-azaspiro[4.4]nonane-8-carboxylate, and 5-(aminomethyl)thiophene-3-carboximidamide HCl based on the procedures set forth in Scheme 199. LC/MS (ESI) m/z: 598 (M+H)⁺. ¹H NMR (400 MHz, DMSO) δ 9.13-8.82 (m, 1H), 8.72 (s, 1H), 8.63 (d, J=6.9 Hz, 1H), 8.57 (s, 1H), 8.36 (s, 1H), 8.28 (s, 1H), 7.91 (d, J=7.0 Hz, 1H), 7.81 (d, J=7.8 Hz, 1H), 7.54 (t, J=10.3 Hz, 2H), 7.33 (d, J=10.8 Hz, 1H), 7.15 (s, 1H), 7.03 (s, 1H), 4.66 (t, J=7.2 Hz, 1H), 4.60-4.06 (m, 4H), 3.84-3.80 (m, 2H), 3.75-3.45 (m, 2H), 3.47-2.93 (m, 3H), 2.37-2.12 (m, 1H), 1.26 (d, J=6.8 Hz, 3H), 1.14 (d, J=6.8 Hz, 1H).

### Scheme 265: Synthesis of (S)-7-(([1,1′-biphenyl]-4-carbonyl)-D-alanyl)-N-((4-carbamimidoylthiophen-2-yl)methyl)-1,4-dioxa-7-azaspiro[4.4]nonane-8-carboxamide (Compound 302)

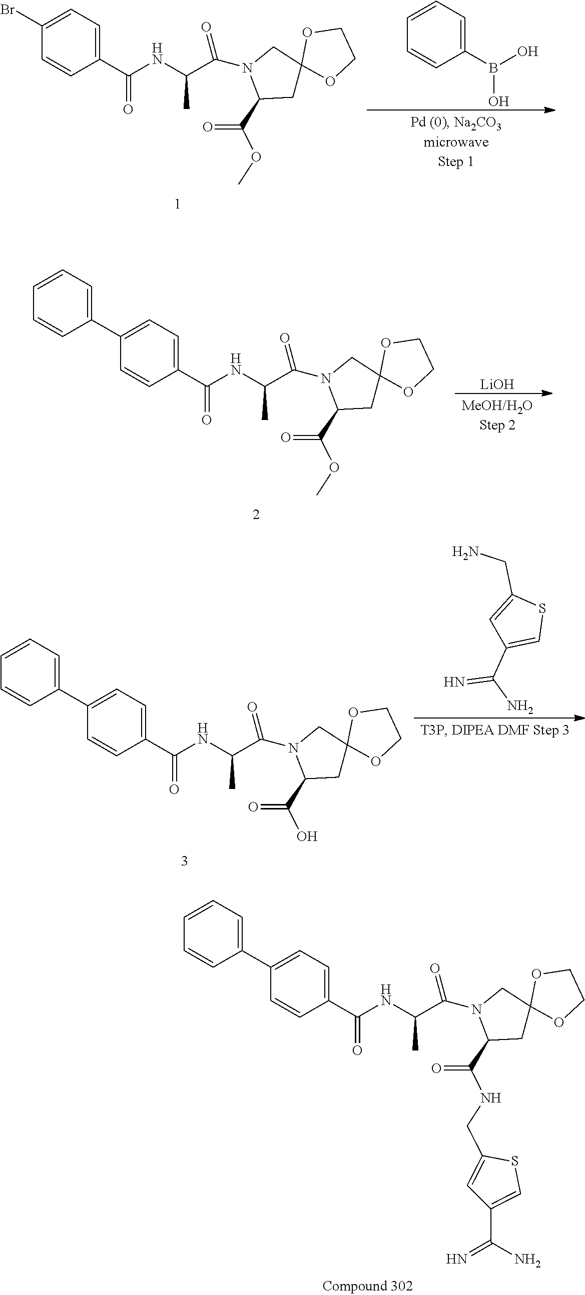

Compound 302 was prepared from phenylboronic acid, methyl (8S)-7-[(2R)-2-benzamidopropanoyl]-1,4-dioxa-7-azaspiro[4.4]nonane-8-carboxylate, and 5-(aminomethyl)thiophene-3-carboximidamide HCl based on the procedures set forth in Scheme 199. LC/MS (ESI) m/z: 562 (M+H)⁺. ¹H NMR (400 MHz, DMSO) δ 9.00 (s, 1H), 8.91 (d, J=5.8 Hz, 1H), 8.77 (d, J=6.6 Hz, 1H), 8.70-8.55 (m, 1H), 8.42 (s, 1H), 8.35 (t, J=6.0 Hz, 1H), 8.04-7.91 (m, 1H), 7.87 (d, J=8.0 Hz, 1H), 7.75 (dd, J=14.7, 7.9 Hz, 2H), 7.50 (t, J=7.6 Hz, 2H), 7.41 (t, J=7.3 Hz, 1H), 7.37-7.24 (m, 1H), 7.13 (d, J=17.0 Hz, 1H), 4.72 (t, J=7.1 Hz, 1H), 4.67-4.42 (m, 2H), 4.39 (t, J=8.0 Hz, 1H), 3.99-3.81 (m, 2H), 3.85-3.53 (m, 1H), 2.49-2.26 (m, 2H), 1.47 (d, J=9.3 Hz, 3H).

### Scheme 266: Synthesis of (S)—N—((R)-1-(4-carbamimidoylthiophen-2-yl)ethyl)-7-(2-(2′,4′-difluoro-[1,1′-biphenyl]-4-yl)acetyl)-1,4-dioxa-7-azaspiro[4.4]nonane-8-carboxamide (Compound 321)

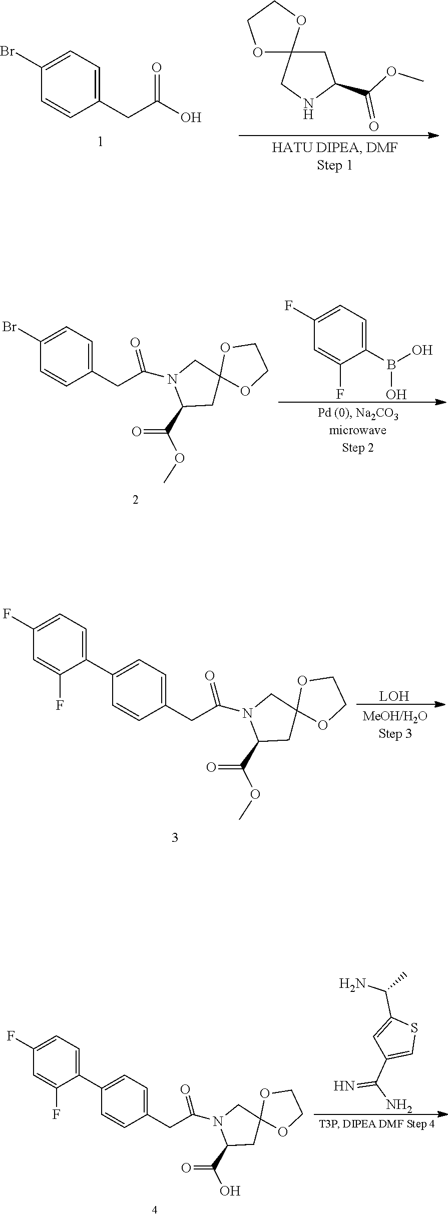

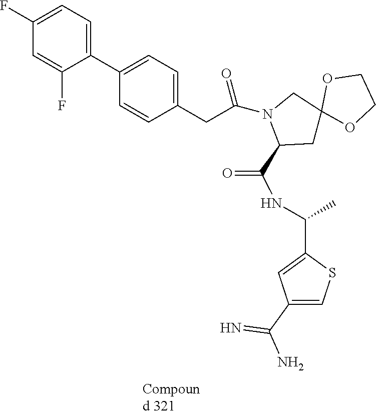 [SMILES: C[C@@H](NC(=O)[C@@H]1CC2(CN1C(=O)Cc1ccc(-c3ccc(F)cc3F)cc1)OCCO2)c1cc(C(=N)N)cs1]

### Step 1: methyl (S)-7-(2-(4-bromophenyl)acetyl)-1,4-dioxa-7-azaspiro[4.4]nonane-8-carboxylate (2)

To a solution of 2-(4-bromophenyl)acetic acid (0.2000 g, 0.9301 mmol, 1.0 equiv.) in DMF (5 mL) was added methyl (8S)-1,4-dioxa-7-azaspiro[4.4]nonane-8-carboxylate (0.2089 g, 1.116 mmol, 1.2 equiv.), HATU (0.4597 g, 1.209 mmol, 1.3 equiv.), and DIPEA (0.4870 mL, 2.79 mmol, 3.0 equiv.). The reaction was stirred at room temperature for 30 minutes. Water (5 mL) was added and the solid collected by filtration and then dried to give methyl (8S)-7-[2-(4-bromophenyl)acetyl]-1,4-dioxa-7-azaspiro[4.4]nonane-8-carboxylate (0.2879 g, 0.7493 mmol, 80.57% yield) as a white solid. LC/MS (ESI) m/z: 385 (M+H)⁺.

### Step 2: methyl (S)-7-(2-(2,4′-difluoro-[1,1′-biphenyl]-4-yl)acetyl)-1,4-dioxa-7-azaspiro[4.4]nonane-8-carboxylate (3)

A mixture of (2,4-difluorophenyl)boronic acid (22.19 mg, 0.1405 mmol, 1.2 equiv.), methyl (8S)-7-[2-(4-bromophenyl)acetyl]-1,4-dioxa-7-azaspiro[4.4]nonane-8-carboxylate (45.00 mg, 0.1171 mmol, 1 equiv.), Pd(dppf)Cl₂·CH₂Cl₂ complex (9.662 mg, 0.01171 mmol, 0.1 equiv.), Na₂CO₃ (24.83 mg, 0.2343 mmol, 2 equiv.) in 1,4-dioxane (3 ml) and water (0.3 mL) was stirred in a microwave reactor at 110° C. for 30 min. The reaction was cooled to room temperature and then filtered through Celite pad. The filtrate extracted with EtOAc, washed with brine, dried over Na₂SO₄, and concentrated. Purification by combi-Flash; 4 g column, solvent A=CH₂Cl₂, solvent B=MeOH. 100% A to 5% B gave methyl (8S)-7-[2-[4-(2,4-difluorophenyl)phenyl]acetyl]-1,4-dioxa-7-azaspiro[4.4]nonane-8-carboxylate (37.45 mg, 0.08972 mmol, 76.60% yield). LC/MS (ESI) m/z: 418 (M+H)⁺.

### Step 3: (S)-7-(2-(2′,4′-difluoro-[1,1′-biphenyl]-4-yl)acetyl)-1,4-dioxa-7-azaspiro[4.4]nonane-8-carboxylic acid (4)

To a solution of methyl (8S)-7-[2-[(4-phenylbenzoyl)amino]acetyl]-1,4-dioxa-7-azaspiro[4.4]nonane-8-carboxylate (30.00 mg, 0.07069 mmol, 1.00 equiv.) in MeOH (1.5 mL) water (0.5 mL) was added LiOH (2.031 mg, 0.08480 mmol, 1.2 equiv.). The reaction was stirred at room temperature overnight. The reaction was concentrated to remove MeOH and then diluted with EtOAc. The pH was adjusted to 1 using 1 N HCl. The two layers were separated, and the aqueous layer was extracted with EtOAc (5 mL×2). The combined organic extracts were washed with brine, dried over Na₂SO₄, and concentrated to give (8S)-7-[2-[(4-phenylbenzoyl)amino]acetyl]-1,4-dioxa-7-azaspiro[4.4]nonane-8-carboxylic acid (26.45 mg, 0.06445 mmol, 91.17% yield) as a white solid. LC/MS (ESI) m/z: 411 (M+H)⁺.

### Step 4: (S)—N—((R)-1-(4-carbamimidoylthiophen-2-yl)ethyl)-7-(2-(2′,4′-difluoro-[1,1′-biphenyl]-4-yl)acetyl)-1,4-dioxa-7-azaspiro[4.4]nonane-8-carboxamide (Compound 321)

To a solution of (8S)-7-[2-[(4-phenylbenzoyl)amino]acetyl]-1,4-dioxa-7-azaspiro[4.4]nonane-8-carboxylic acid (25.00 mg, 0.06092 mmol, 1.0 equiv.) in DMF (1 mL) was added 5-[(1R)-1-aminoethyl]thiophene-3-carboxamidine hydrochloride (15.04 mg, 0.07311 mmol, 1.2 equiv.), T3P, (50 mass %) in ethyl acetate (54.7 μL, 0.0913 mmol, 1.5 equiv.), and DIPEA (31.90 μL, 0.183 mmol, 3.0 equiv.). The reaction mixture was stirred at room temperature for 30 minutes and then purified directly by HPLC to give Compound 321 (4.567 mg, 0.008132 mmol, 13.35% yield). LC/MS (ESI) m/z: 562 (M+H)⁺. ¹H NMR (400 MHz, DMSO) δ 8.45 (d, J=8.4 Hz, 1H), 8.31 (s, 1H), 7.58 (d, J=7.3 Hz, 2H), 7.50-7.38 (m, 2H), 7.33 (d, J=8.1 Hz, 3H), 7.27-7.12 (m, 2H), 5.25-4.99 (m, 1H), 4.61-4.55 (m, 1H), 4.36 (t, J=8.1 Hz, 2H), 3.89-3.80 (m, 2H), 3.78 (d, J=10.9 Hz, 2H), 3.71-3.69 (m, 2H), 3.66-3.54 (m, 2H), 2.39-2.20 (m, 1H), 2.11-1.90 (m, 1H), 1.52 (d, J=6.8 Hz, 3H), 1.27-1.05 (m, 1H).

### Scheme 267: Synthesis of (S)-7-(2-([1,1′-biphenyl]-4-yl)acetyl)-N—((R)-1-(4-carbamimidoylthiophen-2-yl)ethyl)-1,4-dioxa-7-azaspiro[4.4]nonane-8-carboxamide (Compound 338

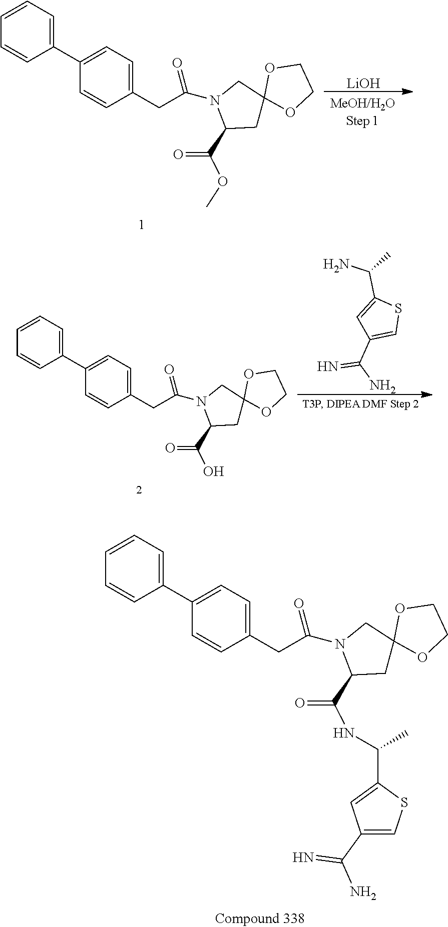

Compound 338 was prepared from methyl (8S)-7-[2-(4-phenylphenyl)acetyl]-1,4-dioxa-7-azaspiro[4.4]nonane-8-carboxylate and 5-[(1R)-1-aminoethyl]thiophene-3-carboxamidine; hydrochloride based on the procedures set forth in Scheme 3. LC/MS (ESI) m/z: 519 (M+H)⁺. ¹H NMR (400 MHz, DMSO) δ 9.03-7.99 (m, 1H), 7.71-7.50 (m, 1H), 7.45 (t, J=7.5 Hz, 1H), 7.34 (dd, J=13.0, 7.3 Hz, 1H), 7.18 (d, J=7.6 Hz, 1H), 5.30-4.61 (m, 1H), 4.36 (t, J=8.1 Hz, 1H), 3.89 (d, J=9.7 Hz, 1H), 3.77-3.70 (m, 1H), 3.60 (d, J=11.1 Hz, 1H), 3.33 (t, J=16.4 Hz, 1H), 2.43-2.13 (m, 1H), 2.03 (dd, J=13.0, 7.6 Hz, 1H), 1.73-1.31 (m, 2H), 1.34-1.10 (m, 1H), 0.88 (d, J=12.2 Hz, 1H).

### Scheme 268: Synthesis of (S)-7-(4-([1,1′-biphenyl]-4-yl)-4-oxobutanoyl)-N—((R)-1-(4-carbamimidoyl thiophen-2-yl)ethyl)-1,4-dioxa-7-azaspiro[4.4]nonane-8-carboxamide (Compound 345)

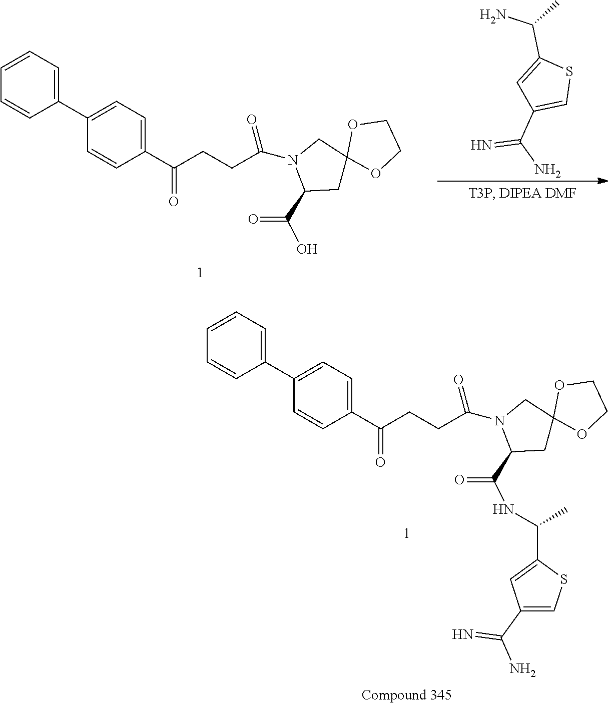 [SMILES: O=C(CCC(=O)N1CC2(C[C@H]1C(=O)O)OCCO2)c1ccc(-c2ccccc2)cc1 ; C[C@@H](N)c1cc(C(=N)N)cs1 ; C[C@@H](NC(=O)[C@@H]1CC2(CN1C(=O)CCC(=O)c1ccc(-c3ccccc3)cc1)OCCO2)c1cc(C(=N)N)cs1]

Compound 345 was prepared from (8S)-7-[4-oxo-4-(4-phenylphenyl)butanoyl]-1,4-dioxa-7-azaspiro[4.4]nonane-8-carboxylic acid and 5-[(1R)-1-aminoethyl]thiophene-3-carboxamidine; hydrochloride based on the procedures set forth in Scheme 66. LC/MS (ESI) m/z: 561 (M+H)⁺. ¹H NMR (400 MHz, DMSO) δ 8.45 (d, J=8.4 Hz, 1H), 8.31 (s, 1H), 7.58 (d, J=7.3 Hz, 2H), 7.50-7.38 (m, 2H), 7.33 (d, J=8.1 Hz, 3H), 7.27-7.12 (m, 2H), 5.25-4.99 (m, 1H), 4.61-4.55 (m, 1H), 4.36 (t, J=8.1 Hz, 2H), 3.89-3.80 (m, 2H), 3.78 (d, J=10.9 Hz, 2H), 3.71-3.69 (m, 2H), 3.66-3.54 (m, 2H), 2.39-2.20 (m, 1H), 2.11-1.90 (m, 1H), 1.52 (d, J=6.8 Hz, 3H), 1.27-1.05 (m, 1H).

### Scheme 269: Synthesis of (S)—N—((R)-1-(4-carbamimidoylthiophen-2-yl)ethyl)-7-(2-(6-(2,4-difluorophenyl)-1-oxoisoindolin-2-yl)acetyl)-1,4-dioxa-7-azaspiro[4.4]nonane-8-carboxamide (Compound 346)

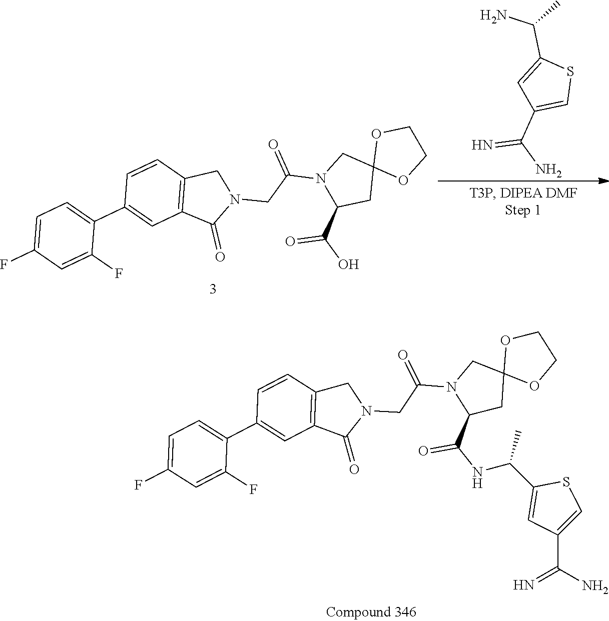 [SMILES: O=C(O)[C@@H]1CC2(CN1C(=O)CN1Cc3ccc(-c4ccc(F)cc4F)cc3C1=O)OCCO2 ; C[C@@H](N)c1cc(C(=N)N)cs1 ; C[C@@H](NC(=O)[C@@H]1CC2(CN1C(=O)CN1Cc3ccc(-c4ccc(F)cc4F)cc3C1=O)OCCO2)c1cc(C(=N)N)cs1]

Compound 346 was prepared from (8S)-7-[2-[6-(2,4-difluorophenyl)-1-oxo-isoindolin-2-yl]acetyl]-1,4-dioxa-7-azaspiro[4.4]nonane-8-carboxylic acid and 5-[(1R)-1-aminoethyl]thiophene-3-carboxamidine; hydrochloride based on the procedures set forth in Scheme 66. LC/MS (ESI) m/z: 610 (M+H)⁺. ¹H NMR (400 MHz, DMSO) δ 8.65 (d, J=7.4 Hz, 1H), 8.43 (s, 1H), 7.97 (dd, J=8.5, 5.3 Hz, 1H), 7.87-7.62 (m, 1H), 7.33 (t, J=8.7 Hz, 1H), 7.20 (d, J=3.6 Hz, 1H), 7.09 (d, J=3.6 Hz, 1H), 4.71 (s, 1H), 4.55 (d, J=5.4 Hz, 1H), 4.47 (d, J=5.8 Hz, 2H), 4.39 (t, J=7.8 Hz, 1H), 4.17 (dd, J=16.8, 5.3 Hz, 2H), 4.04-3.83 (m, 4H), 3.80 (d, J=10.9 Hz, 1H), 3.62 (d, J=10.8 Hz, 1H), 2.32 (dd, J=13.3, 8.6 Hz, 1H), 2.05 (dd, J=13.2, 6.9 Hz, 1H).

### Scheme 270: (S)—N—((R)-1-(4-carbamimidoylthiophen-2-yl)ethyl)-7-((3-fluoro-5-phenylpicolinoyl)glycyl)-1,4-dioxa-7-azaspiro[4.4]nonane-8-carboxamide (Compound 347)

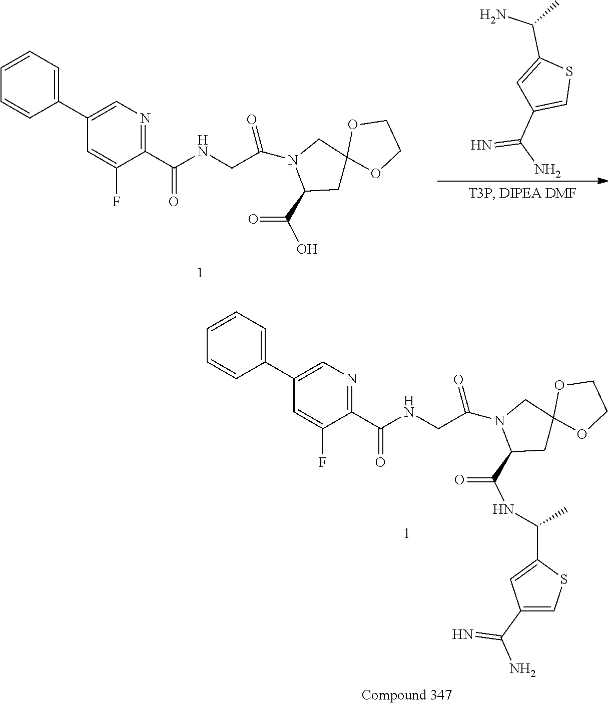 [SMILES: O=C(NCC(=O)N1CC2(C[C@H]1C(=O)O)OCCO2)c1ncc(-c2ccccc2)cc1F ; C[C@@H](N)c1cc(C(=N)N)cs1 ; C[C@@H](NC(=O)[C@@H]1CC2(CN1C(=O)CNC(=O)c1ncc(-c3ccccc3)cc1F)OCCO2)c1cc(C(=N)N)cs1]

Compound 347 was prepared from (8S)-7-[2-[(3-fluoro-5-phenyl-pyridine-2-carbonyl)amino]acetyl]-1,4-dioxa-7-azaspiro[4.4]nonane-8-carboxylic acid and 5-[(1R)-1-aminoethyl]thiophene-3-carboxamidine; hydrochloride based on the procedures set forth in Scheme 66. LC/MS (ESI) m/z:581 (M+H)⁺. ¹H NMR (400 MHz, DMSO) δ 8.81 (s, 1H), 8.65 (s, 1H), 8.38 (s, 1H), 8.33 (d, J=8.1 Hz, 1H), 8.26 (s, 1H), 8.15 (d, J=12.2 Hz, 1H), 7.81 (d, J=7.4 Hz, 1H), 7.47 (dt, J=13.2, 7.2 Hz, 2H), 5.08 (t, J=7.3 Hz, 1H), 4.33 (t, J=7.8 Hz, 1H), 4.20-4.06 (m, 1H), 4.05-3.93 (m, 1H), 3.87 (dd, J=17.7, 6.2 Hz, 3H), 3.75-3.59 (m, 1H), 3.55 (d, J=10.7 Hz, 1H), 2.27 (t, J=11.1 Hz, 1H), 2.08-1.84 (m, 1H), 1.47 (d, J=6.4 Hz, 1H), 1.13-0.95 (m, 1H).

### Scheme 271: Synthesis of (S)—N—((R)-1-(4-carbamimidoylthiophen-2-yl)ethyl)-7-((5-(2,4-difluorophenyl)-3-fluoropicolinoyl)glycyl)-1,4-dioxa-7-azaspiro[4.4]nonane-8-carboxamide (Compound 356)

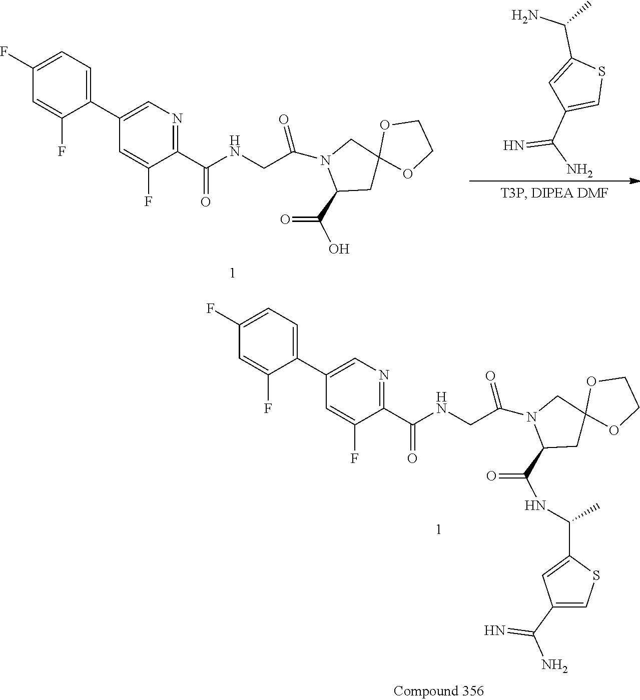 [SMILES: O=C(NCC(=O)N1CC2(C[C@H]1C(=O)O)OCCO2)c1ncc(-c2ccc(F)cc2F)cc1F ; C[C@@H](N)c1cc(C(=N)N)cs1 ; C[C@@H](NC(=O)[C@@H]1CC2(CN1C(=O)CNC(=O)c1ncc(-c3ccc(F)cc3F)cc1F)OCCO2)c1cc(C(=N)N)cs1]

Compound 356 was prepared from (8S)-7-[2-[[5-(2,4-difluorophenyl)-3-fluoro-pyridine-2-carbonyl]amino]acetyl]-1,4-dioxa-7-azaspiro[4.4]nonane-8-carboxylic acid and 5-[(1R)-1-aminoethyl]thiophene-3-carboxamidine; hydrochloride based on the procedures set forth in Scheme 66. LC/MS (ESI) m/z: 617(M+H)⁺. ¹H NMR (400 MHz, DMSO) δ 8.76 (s, 1H), 8.71 (s, 1H), 8.48 (s, 1H), 8.38 (d, J=8.2 Hz, 1H), 8.25 (s, 1H), 8.11 (d, J=11.8 Hz, 1H), 7.81 (q, J=8.2 Hz, 1H), 7.62-7.41 (m, 2H), 7.31 (t, J=8.4 Hz, 1H), 5.24-5.04 (m, 1H), 4.40 (t, J=7.8 Hz, 1H), 4.24-4.00 (m, 1H), 4.02-3.84 (m, 3H), 3.84-3.69 (m, 1H), 3.62 (d, J=10.6 Hz, 1H), 2.39-2.25 (m, 1H), 2.05 (dd, J=13.2, 6.8 Hz, 1H), 1.53 (d, J=6.9 Hz, 1H), 1.46 (d, J=6.9 Hz, 3H), 0.94-0.74 (m, 1H).

### Scheme 272: Synthesis of (S)—N—((R)-1-(4-carbamimidoylthiophen-2-yl)ethyl)-7-(2-(1-oxo-5-phenylisoindolin-2-yl)acetyl)-1,4-dioxa-7-azaspiro[4.4]nonane-8-carboxamide (Compound 365)

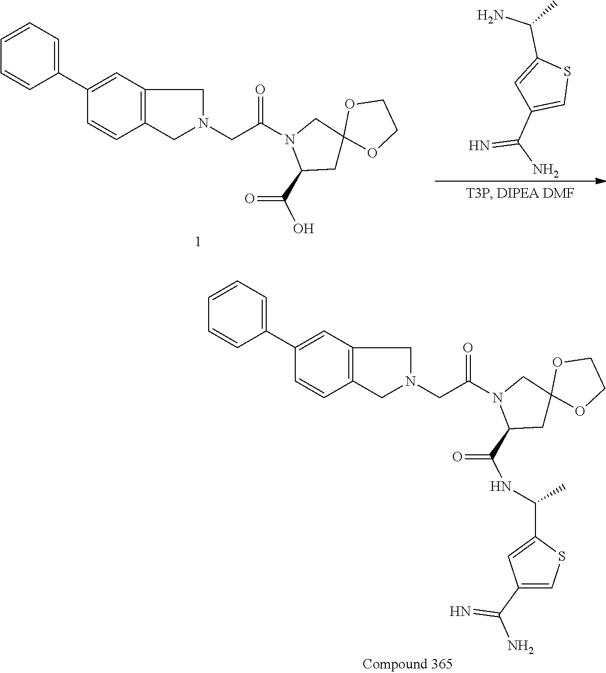 [SMILES: O=C(O)[C@@H]1CC2(CN1C(=O)CN1Cc3ccc(-c4ccccc4)cc3C1)OCCO2 ; C[C@@H](N)c1cc(C(=N)N)cs1 ; C[C@@H](NC(=O)[C@@H]1CC2(CN1C(=O)CN1Cc3ccc(-c4ccccc4)cc3C1)OCCO2)c1cc(C(=N)N)cs1]

Compound 365 was prepared from (8S)-7-[2-(1-oxo-5-phenyl-isoindolin-2-yl)acetyl]-1,4-dioxa-7-azaspiro[4.4]nonane-8-carboxylic acid and 5-[(1R)-1-aminoethyl]thiophene-3-carboxamidine; hydrochloride based on the procedures set forth in Scheme 66. LC/MS (ESI) m/z: 574 (M+H)⁺. ¹H NMR (400 MHz, DMSO) δ 8.46 (d, J=17.0 Hz, 1H), 8.28 (s, 1H), 7.90 (d, J=10.2 Hz, 1H), 7.84-7.70 (m, 1H), 7.51 (t, J=7.5 Hz, 1H), 7.43 (t, J=7.4 Hz, OH), 4.70-4.43 (m, 1H), 4.42-4.23 (m, 1H), 4.11-3.84 (m, 2H), 3.80 (d, J=10.7 Hz, OH), 3.75-3.57 (m, 0H), 2.50 (t, J=2.4 Hz, 18H), 2.43-2.24 (m, 0H), 2.03 (dd, J=13.4, 7.4 Hz, OH), 1.57 (d, J=7.0 Hz, 0H), 1.46 (d, J=6.9 Hz, 1H).

### Scheme 273: Synthesis of (S)—N—((R)-1-(4-carbamimidoylthiophen-2-yl)ethyl)-7-(2-(5-(3,4-difluorophenyl)-1-oxoisoindolin-2-yl)acetyl)-1,4-dioxa-7-azaspiro[4.4]nonane-8-carboxamide (Compound 366)

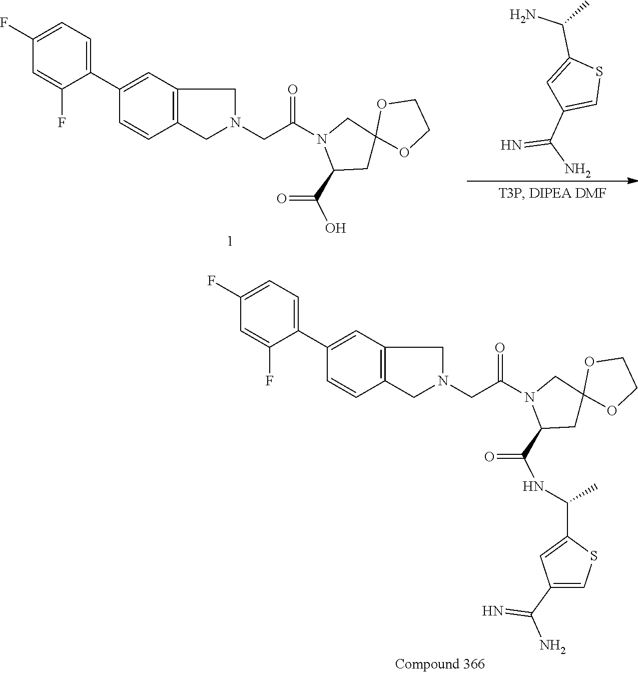 [SMILES: O=C(O)[C@@H]1CC2(CN1C(=O)CN1Cc3ccc(-c4ccc(F)cc4F)cc3C1)OCCO2 ; C[C@@H](N)c1cc(C(=N)N)cs1 ; C[C@@H](NC(=O)[C@@H]1CC2(CN1C(=O)CN1Cc3ccc(-c4ccc(F)cc4F)cc3C1)OCCO2)c1cc(C(=N)N)cs1]

Compound 366 was prepared from (8S)-7-[2-[5-(2,4-difluorophenyl)-1-oxo-isoindolin-2-yl]acetyl]-1,4-dioxa-7-azaspiro[4.4]nonane-8-carboxylic acid and 5-[(1R)-1-aminoethyl]thiophene-3-carboxamidine; hydrochloride based on the procedures set forth in Scheme 66. LC/MS (ESI) m/z: 610 (M+H)⁺. ¹H NMR (400 MHz, DMSO) δ 8.49 (s, 1H), 8.45 (d, J=8.1 Hz, 1H), 8.26 (s, 1H), 7.83-7.76 (m, 1H), 7.65 (t, J=8.1 Hz, 1H), 7.52 (s, 1H), 7.46-7.36 (m, 1H), 7.24 (t, J=8.0 Hz, 1H), 6.89 (s, 1H), 5.13 (t, J=7.4 Hz, 1H), 4.59 (s, 1H), 4.56-4.45 (m, 1H), 4.35 (s, 1H), 4.39-4.29 (m, 1H), 4.25 (s, 1H), 3.97-3.92 (m, 3H), 3.91 (d, J=16.3 Hz, 1H), 3.80 (d, J=10.8 Hz, 1H), 3.70-3.55 (m, 2H), 2.04 (dt, J=13.3, 6.4 Hz, 1H), 1.59-1.48 (m, 3H), 1.46-1.24 (m, 1H), 1.14 (d, J=14.4 Hz, 1H), 0.90-0.86 (m, 1H).

### Scheme 274: Synthesis of (2S,4R)—N—((R)-1-(4-carbamimidoylthiophen-2-yl)ethyl)-4-(difluoromethoxy)-1-(2-(1-oxo-5-phenylisoindolin-2-yl)acetyl)pyrrolidine-2-carboxamide (Compound 385)

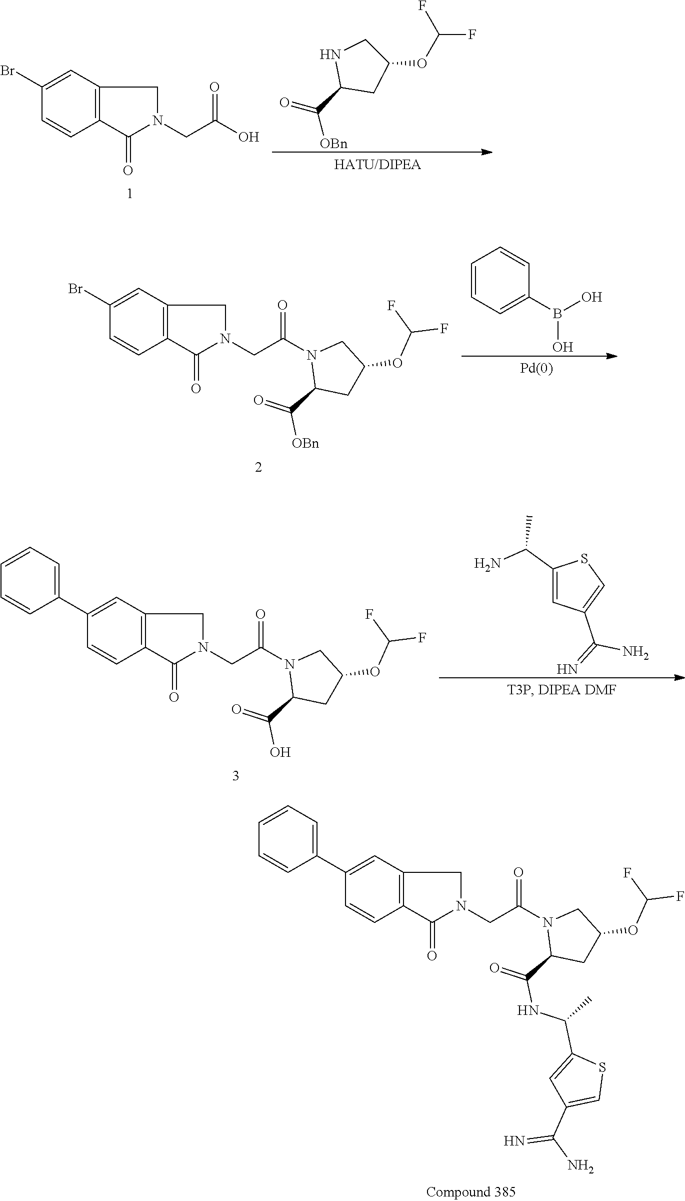

Compound 385 was prepared from 2-(5-bromo-1-oxo-isoindolin-2-yl)acetic acid, benzyl (2S,4R)-4-(difluoromethoxy)pyrrolidine-2-carboxylate, phenylboronic acid, and 5-[(1R)-1-aminoethyl]thiophene-3-carboxamidine; hydrochloride based on the procedures set forth in Scheme 12. LC/MS (ESI) m/z: 582 (M+H)⁺. ¹H NMR (400 MHz, DMSO) δ 9.02-8.21 (m, 1H), 7.91 (s, 1H), 7.77 (d, J=14.7 Hz, 3H), 7.62-7.36 (m, 2H), 7.05-6.58 (m, 1H), 5.20-4.83 (m, 2H), 4.68-4.31 (m, 3H), 4.04-3.52 (m, 1H), 3.46-3.11 (m, 2H), 2.33-1.94 (m, 2H), 1.47-1.35 (m, 2H), 1.27-1.05 (m, 1H), 0.89 (t, J=7.3 Hz, 1H),

### Scheme 275: Synthesis of (S)—N—((R)-1-(4-carbamimidoylthiophen-2-yl)ethyl)-7-(2-(1-oxo-6-phenylisoindolin-2-yl)acetyl)-1,4-dioxa-7-azaspiro[4.4]nonane-8-carboxamide (Compound 386)

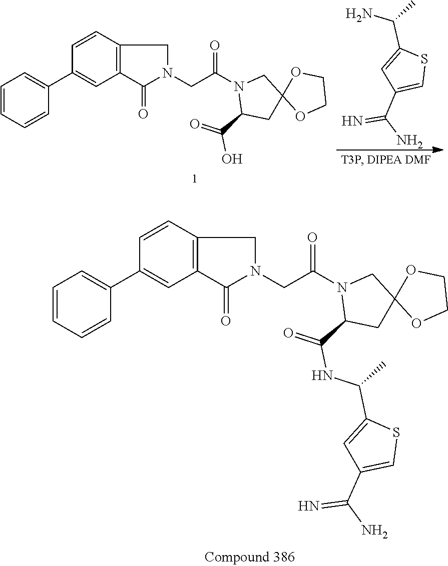

Compound 386 was prepared from (8S)-7-[2-(1-oxo-6-phenyl-isoindolin-2-yl)acetyl]-1,4-dioxa-7-azaspiro[4.4]nonane-8-carboxylic acid and 5-[(1R)-1-aminoethyl]thiophene-3-carboxamidine; hydrochloride based on the procedures set forth in Scheme 66. LC/MS (ESI) m/z: 574 (M+H)⁺. ¹H NMR (400 MHz, DMSO) δ 8.47 (s, 1H), 8.41 (s, 1H), 7.93 (d, J=18.0 Hz, 1H), 7.91-7.61 (m, 2H), 7.51 (t, J=7.4 Hz, 2H), 7.42 (t, J=7.3 Hz, 1H), 7.38 (d, J=7.1 Hz, 1H), 7.32 (t, J=7.2 Hz, 1H), 4.65-4.24 (m, 4H), 4.13-3.85 (m, 4H), 3.79 (d, J=10.7 Hz, 1H), 3.65 (dd, J=16.1, 11.5 Hz, 1H), 3.30-2.95 (m, 4H), 2.33 (t, J=11.0 Hz, 1H), 2.17-1.99 (m, 1H), 1.81-1.19 (m, 3H).

### Scheme 276: Synthesis of (1S,3S,5S)—N-(2,3-dichlorobenzyl)-5-methyl-2-((4-phenoxybenzoyl)glycyl)-2-azabicyclo[3.1.0]hexane-3-carboxamide (Compound 387)

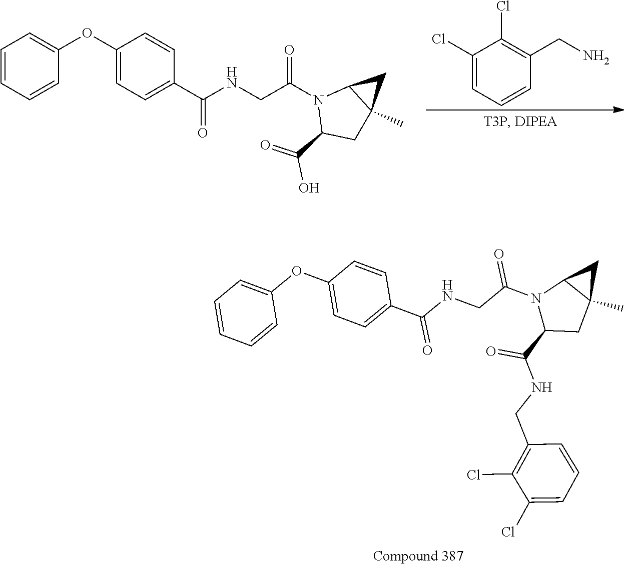 [SMILES: C[C@@]12C[C@@H]1N(C(=O)CNC(=O)c1ccc(Oc3ccccc3)cc1)[C@H](C(=O)O)C2 ; NCc1cccc(Cl)c1Cl ; C[C@@]12C[C@@H]1N(C(=O)CNC(=O)c1ccc(Oc3ccccc3)cc1)[C@H](C(=O)NCc1cccc(Cl)c1Cl)C2]

Compound 387 was prepared from (1 S,3S,5S)-5-methyl-2-[2-[(4-phenoxybenzoyl)amino]acetyl]-2-azabicyclo[3.1.0]hexane-3-carboxylic acid and 2,3-dichlorophenyl)methanamine hydrochloride based on the procedures set forth in Scheme 66. LC/MS (ESI) m/z: 552(M+H)+. ¹H NMR (400 MHz, DMSO) δ 8.60 (t, J=5.7 Hz, 1H), 8.33 (t, J=6.0 Hz, 1H), 7.80 (d, J=8.2 Hz, 2H), 7.44 (d, J=7.6 Hz, 1H), 7.37 (t, J=7.7 Hz, 2H), 7.26 (tq, J=15.4, 7.6 Hz, 2H), 7.15 (t, J=7.4 Hz, 1H), 7.02 (d, J=8.0 Hz, 2H), 6.97 (t, J=6.9 Hz, 1H), 4.67 (dd, J=11.4, 3.2 Hz, 1H), 4.42-4.14 (m, 2H), 3.96 (dd, J=16.5, 5.6 Hz, 1H), 3.50-3.38 (m, 1H), 3.45-3.25 (m, 2H), 2.25 (t, J=12.4 Hz, 1H), 1.95 (dd, J=13.2, 3.2 Hz, 1H), 1.16 (d, J=12.1 Hz, 1H), 0.62 (t, J=5.6 Hz, 1H).

### Scheme 277: Synthesis of (S)—N—((R)-1-(4-carbamimidoylthiophen-2-yl)ethyl)-7-((3-fluoro-5-(p-tolyl)picolinoyl)glycyl)-1,4-dioxa-7-azaspiro[4.4]nonane-8-carboxamide (Compound 388)

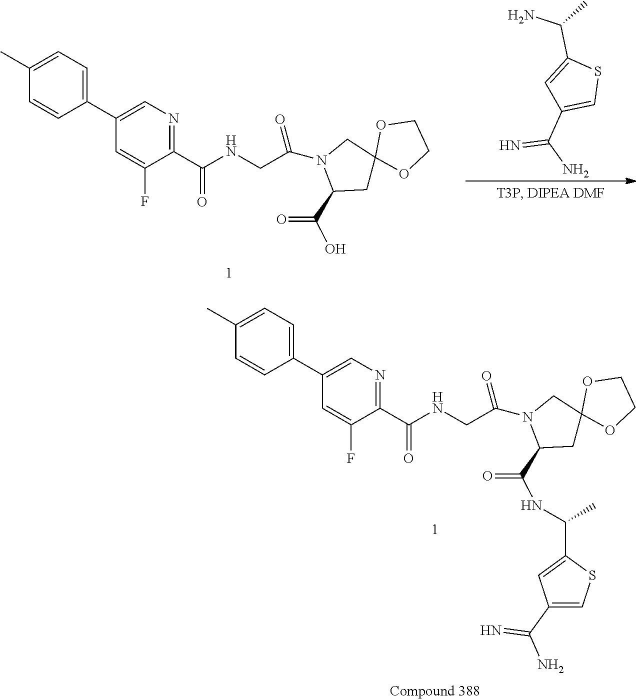 [SMILES: Cc1ccc(-c2cnc(C(=O)NCC(=O)N3CC4(C[C@H]3C(=O)O)OCCO4)c(F)c2)cc1 ; C[C@@H](N)c1cc(C(=N)N)cs1 ; Cc1ccc(-c2cnc(C(=O)NCC(=O)N3CC4(C[C@H]3C(=O)N[C@H](C)c3cc(C(=N)N)cs3)OCCO4)c(F)c2)cc1]

Compound 388 was prepared from (8S)-7-[2-[[3-fluoro-5-(p-tolyl)pyridine-2-carbonyl]amino]acetyl]-1,4-dioxa-7-azaspiro[4.4]nonane-8-carboxylic acid and 5-[(1R)-1-aminoethyl]thiophene-3-carboxamidine; hydrochloride based on the procedures set forth in Scheme 66. LC/MS (ESI) m/z: 595 (M+H)⁺. ¹H NMR (400 MHz, DMSO) δ 8.79 (s, 1H), 8.63 (s, 1H), 8.34 (d, J=8.2 Hz, 1H), 8.11 (d, J=12.3 Hz, 1H), 7.71 (d, J=7.8 Hz, 1H), 7.50 (s, 1H), 7.29 (d, J=7.8 Hz, 1H), 6.20 (s, 1H), 5.37-4.90 (m, 1H), 4.34 (t, J=7.9 Hz, 1H), 4.20-3.90 (m, 2H), 3.98-3.71 (m, 4H), 3.74-3.62 (m, 1H), 3.55 (d, J=10.9 Hz, 1H), 3.31 (d, J=12.0 Hz, 1H), 2.31 (s, 3H), 1.99 (dd, J=13.5, 6.8 Hz, 2H), 1.63-1.35 (m, 3H), 1.17-1.15 (m, 1H).

### Scheme 278: Synthesis of (1S,3S,5S)—N—((R)-1-(4-carbamimidoylthiophen-2-yl)ethyl)-2-((9,9-difluoro-9H-fluorene-3-carbonyl)glycyl)-5-(morpholinomethyl)-2-azabicyclo [3.1.0]hexane-3-carboxamide (Compound 267)

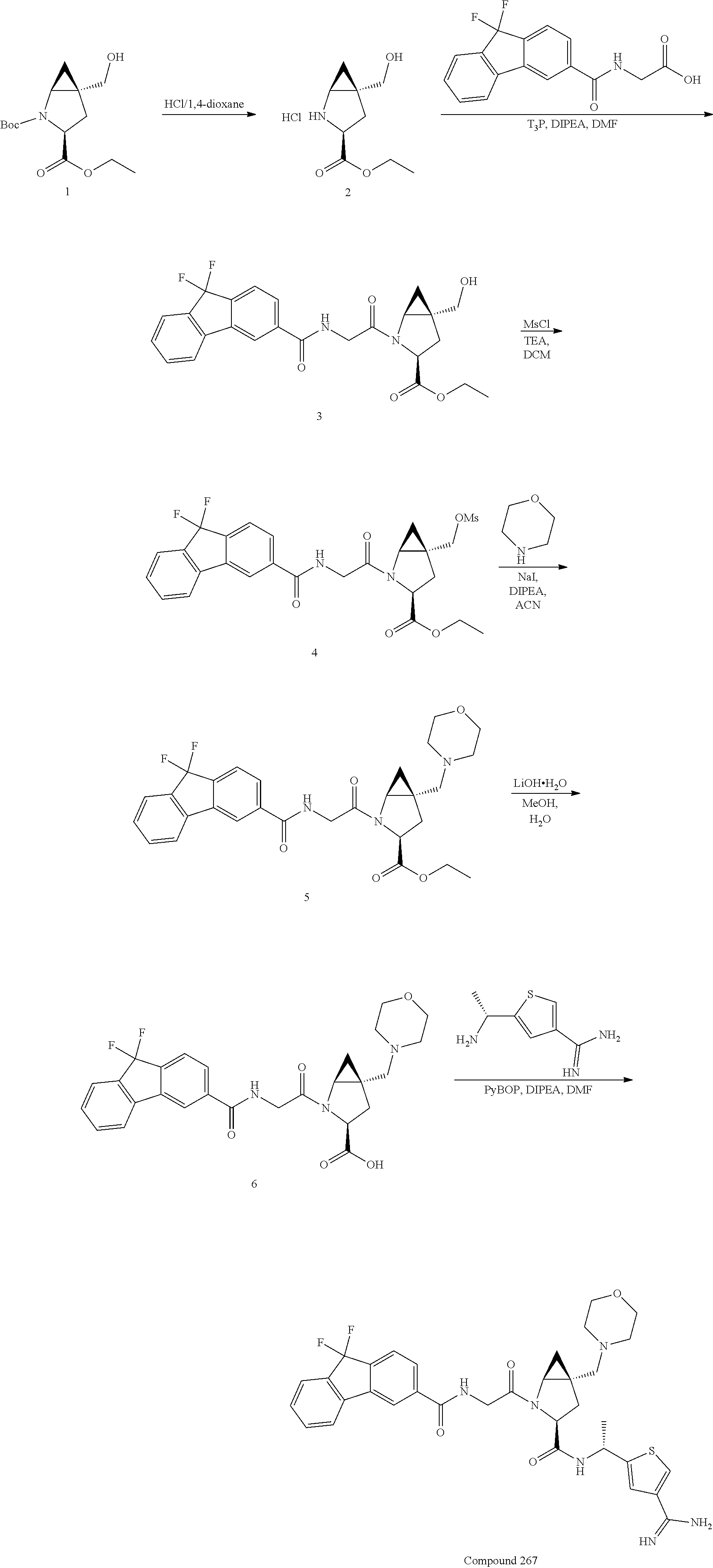

### Step 1: ethyl (1S,3S,5R)-5-(hydroxymethyl)-2-azabicyclo[3.1.0]hexane-3-carboxylate hydrochloride

A solution of 2-(tert-butyl) 3-ethyl (1S,3S,5R)-5-(hydroxymethyl)-2-azabicyclo[3.1.0]hexane-2,3-dicarboxylate (200 mg, 0.70 mmol) in HCl/1,4-dioxane (3 mL, 4M) was stirred at room temperature for 1 hour. The mixture was concentrated to dryness under reduced pressure to give ethyl (1S,3S,5R)-5-(hydroxymethyl)-2-azabicyclo[3.1.0]hexane-3-carboxylate hydrochloride (143 mg, yield 92.3%) as a colorless oil, which was directly used in the next reaction without further purification. LC/MS (ESI) m/z: 186 (M+H)⁺.

### Step 2: ethyl (1S,3S,5R)-2-((9,9-difluoro-9H-fluorene-3-carbonyl)glycyl)-5-(hydroxymethyl)-2-azabicyclo[3.1.0]hexane-3-carboxylate

To a mixture of (9,9-difluoro-9H-fluorene-3-carbonyl)glycine (300 mg, 1.0 mmol) and ethyl (1S,3S,5R)-5-(hydroxymethyl)-2-azabicyclo[3.1.0]hexane-3-carboxylate hydrochloride (222 mg, 1.0 mmol) in DMF (3 mL) was added T₃P (1.81 g, 3.0 mmol, 50% wt. in EtOAc) and DIPEA (0.98 mL, 6.0 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-40% EtOAc in PE) to give ethyl (1S,3S,5R)-2-((9,9-difluoro-9H-fluorene-3-carbonyl)glycyl)-5-(hydroxymethyl)-2-azabicyclo [3.1.0]hexane-3-carboxylate (166 mg, yield 35.7%) as a yellow solid. LCMS (ESI) m/z: 471 (M+H)⁺.

### Step 3: ethyl (1S,3S,5R)-2-((9,9-difluoro-9H-fluorene-3-carbonyl)glycyl)-5-(((methylsulfonyl) oxy)methyl)-2-azabicyclo[3.1.0]hexane-3-carboxylate

To a mixture of ethyl (1S,3S,5R)-2-((9,9-difluoro-9H-fluorene-3-carbonyl)glycyl)-5-(hydroxymethyl)-2-azabicyclo[3.1.0]hexane-3-carboxylate (50 mg, 0.11 mmol) and TEA (86 mg, 0.0.88 mmol) in DCM (2 mL) was added MsCl (37 mg, 0.33 mmol) at 0° C. and the mixture was stirred at room temperature for 1 hour. The mixture was diluted with EtOAc, washed with water and brine, dried over anhydrous Na₂SO₄, filtered, and concentrated under reduced pressure to dryness to give ethyl (1 S,3S,5R)-2-((9,9-difluoro-9H-fluorene-3-carbonyl)glycyl)-5-(((methylsulfonyl)oxy)methyl)-2-azabicyclo [3.1.0]hexane-3-carboxylate (55 mg, yield 94.8%) as a brown oil, which was used directly in the next reaction. LCMS (ESI) (m/z): 549 (M+H)⁺.

### Step 4: ethyl (1S,3S,5S)-2-((9,9-difluoro-9H-fluorene-3-carbonyl)glycyl)-5-(morpholinomethyl)-2-azabicyclo[3.1.0]hexane-3-carboxylate

To a mixture of ethyl (1S,3S,5R)-2-((9,9-difluoro-9H-fluorene-3-carbonyl)glycyl)-5-(((methylsulfonyl)oxy)methyl)-2-azabicyclo[3.1.0]hexane-3-carboxylate (70 mg, 0.13 mmol) and morpholine (33 mg, 0.39 mmol) in CH₃CN (3 mL) was added DIPEA (148 mg, 1.17 mmol) and NaI (21 mg, 0.14 mmol) at 0° C. and the mixture was stirred under N₂ atmosphere at room temperature for 16 hours. The mixture was diluted with EtOAc, washed with water and brine, dried over anhydrous Na₂SO₄, filtered, and concentrated under reduced pressure to dryness. The residue was purified by prep-TLC (DCM:MeOH=20:1) to give ethyl (1S,3S,5S)-2-((9,9-difluoro-9H-fluorene-3-carbonyl)glycyl)-5-(morpholinomethyl)-2-azabicyclo[3.1.0]hexane-3-carboxylate (21 mg, yield 30.9%) as a brown oil. LCMS (ESI) (m/z): 540 (M+H)⁺.

### Step 5: (1S,3S,5S)-2-((9,9-difluoro-9H-fluorene-3-carbonyl)glycyl)-5-(morpholinomethyl)-2-azabicyclo[3.1.0]hexane-3-carboxylic acid

To a solution of ethyl (1 S,3S,5S)-2-((9,9-difluoro-9H-fluorene-3-carbonyl)glycyl)-5-(morpholinomethyl)-2-azabicyclo[3.1.0]hexane-3-carboxylate (21 mg, 0.04 mmol) in MeOH (1.0 mL) and water (0.5 mL) was added LiOH·H₂O (3.3 mg, 0.08 mmol) at 0° C. and the mixture was stirred at room temperature for 1 hour. The mixture was acidified with 1 N aq·HCl to pH ˜3 and extracted with EtOAc twice. The combined organic layers were washed with brine, dried over anhydrous Na₂SO₄, filtered, and concentrated under reduced pressure to dryness to give (1S,3S,5S)-2-((9,9-difluoro-9H-fluorene-3-carbonyl)glycyl)-5-(morpholinomethyl)-2-azabicyclo[3.1.0]hexane-3-carboxylic acid (17 mg, yield 85.0%) as a yellow oil, which was used directly in the next reaction. LCMS (ESI) (m/z): 512 (M+H)⁺.

### Step 6: (1S,3S,5S)—N—((R)-1-(4-carbamimidoylthiophen-2-yl)ethyl)-2-((9,9-difluoro-9H-fluorene-3-carbonyl)glycyl)-5-(morpholinomethyl)-2-azabicyclo[3.1.0]hexane-3-carboxamide (Compound 267)

To a mixture of (1S,3S,5S)-2-((9,9-difluoro-9H-fluorene-3-carbonyl)glycyl)-5-(morpholinomethyl)-2-azabicyclo[3.1.0]hexane-3-carboxylic acid (17 mg, 0.03 mmol) and (R)-5-(1-aminoethyl)thiophene-3-carboximidamide (21 mg, 0.09 mmol) in DMF (2.0 mL) was added DIPEA (39 mg, 0.27 mmol) and PyBOP (26 mg, 0.05 mmol) at 0° C. and the mixture was stirred under N₂ atmosphere at room temperature for 2 hours. The mixture was concentrated under reduced pressure to dryness. The residue was purified by prep-TLC (DCM:MeOH=10:1) and further purified by prep-HPLC to give Compound 267 (2.0 mg, yield 9.1%) as a white solid. ¹H NMR (400 MHz, CD₃OD) δ 8.47 (s, 2H), 8.20 (d, J=7.3 Hz, 2H), 7.88 (d, J=7.9 Hz, 1H), 7.74 (dd, J=13.9, 7.8 Hz, 2H), 7.66 (d, J=7.8 Hz, 1H), 7.58 (t, J=7.6 Hz, 1H), 7.50 (s, 1H), 7.45 (t, J=7.5 Hz, 1H), 5.23 (q, J=6.6 Hz, 1H), 4.91 (s, 1H), 4.51 (d, J=16.5 Hz, 1H), 4.28 (d, J=16.6 Hz, 1H), 3.72 (t, J=4.3 Hz, 4H), 3.53-3.48 (m, 1H), 2.76 (t, J=12.5 Hz, 1H), 2.64 (d, J=12.8 Hz, 1H), 2.58-2.46 (m, 4H), 2.39 (d, J=12.8 Hz, 1H), 2.10 (dd, J=13.7, 3.5 Hz, 1H), 1.58 (d, J=7.0 Hz, 3H), 1.41 (dd, J=5.2, 2.1 Hz, 1H), 0.95 (t, J=5.6 Hz, 1H). LCMS (ESI) (m/z): 663 (M+H)⁺.

### Scheme 279: Synthesis of (2S,4R)—N—((R)-1-(4-carbamimidoylthiophen-2-yl)ethyl)-1-((9,9-difluoro-9H-fluorene-3-carbonyl)glycyl)-4-fluoro-4-methylpyrrolidine-2-carboxamide (Compound 268)

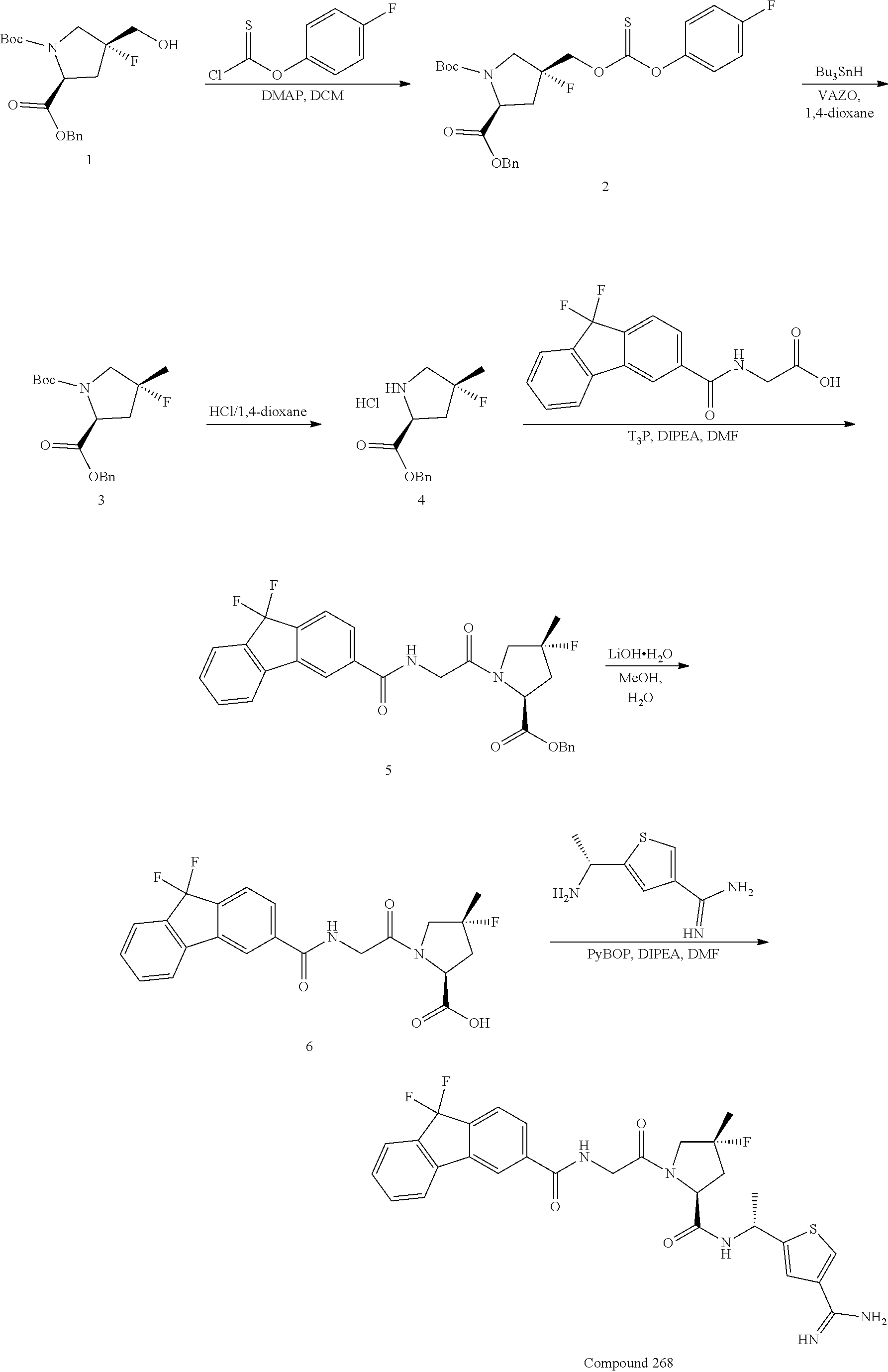

### Step 1: 2-benzyl 1-(tert-butyl) (2S,4R)-4-fluoro-4-((((4-fluorophenoxy)carbonothioyl) oxy)methyl)pyrrolidine-1,2-dicarboxylate

To a mixture of 2-benzyl 1-(tert-butyl) (2S,4R)-4-fluoro-4-(hydroxymethyl)pyrrolidine-1,2-dicarboxylate (500 mg, 1.81 mmol) and O-(4-fluorophenyl) carbonochloridothioate (514 mg, 2.72 mmol) in DCM (6 mL) was added DMAP (661 mg, 5.43 mmol) and the solution was stirred at 25° C. for 5 hours. The mixture was diluted with EtOAc, washed with 0.5N aq·HCl and brine, dried over anhydrous Na₂SO₄, filtered, and concentrated under reduced pressure to dryness. The residue was purified by flash chromatography (silica gel, 0-12% EtOAc in PE) to give 2-benzyl 1-(tert-butyl) (2S,4R)-4-fluoro-4-((((4-fluorophenoxy) carbonothioyl)oxy)methyl) pyrrolidine-1,2-dicarboxylate (573 mg, yield 62.6%) as a colorless oil. LCMS (ESI) m/z: 508 (M+H)⁺.

### Step 2: 2-benzyl 1-(tert-butyl) (2S,4R)-4-fluoro-4-methylpyrrolidine-1,2-dicarboxylate

To a mixture of 2-benzyl 1-(tert-butyl) (2S,4R)-4-fluoro-4-((((4-fluorophenoxy) carbonothioyl) oxy)methyl)pyrrolidine-1,2-dicarboxylate (573 mg, 1.13 mmol) and tributylstannane (0.76 mL, 2.82 mmol) in 1,4-dioxane (6 mL) was added VAZO (93 mg, 0.57 mmol) and the reaction mixture was stirred at 105° C. under N₂ atmosphere for 0.5 hour. The mixture was quenched with saturated aq·KF and extracted with EtOAc twice. The combined organic layers were washed with brine, dried over anhydrous Na₂SO₄, filtered, and concentrated under reduced pressure to dryness. The residue was purified by flash chromatography (silica gel, 0-20% EtOAc in PE) to give 2-benzyl 1-(tert-butyl) (2S,4R)-4-fluoro-4-methylpyrrolidine-1,2-dicarboxylate (200 mg, yield 52.6%) as a light-yellow oil. LCMS (ESI) m/z: 338 (M+H)⁺.

### Step 3: benzyl (2S,4R)-4-fluoro-4-methylpyrrolidine-2-carboxylate hydrochloride

A solution of 2-benzyl 1-(tert-butyl) (2S,4R)-4-fluoro-4-methylpyrrolidine-1,2-dicarboxylate (200 mg, 0.59 mmol) in HCl/1,4-dioxane (3 mL, 4M) was stirred at room temperature for 1 hour. The mixture was concentrated to dryness under reduced pressure to give benzyl (2S,4R)-4-fluoro-4-methylpyrrolidine-2-carboxylate hydrochloride (138 mg, yield 85.7%) as a brown oil, which was directly used in the next reaction without further purification. LC/MS (ESI) m/z: 238 (M+H)⁺.

### Step 4: benzyl (2S,4R)-1-((9,9-difluoro-9H-fluorene-3-carbonyl)glycyl)-4-fluoro-4-methylpyrrolidine-2-carboxylate

To a mixture of (9,9-difluoro-9H-fluorene-3-carbonyl)glycine (100 mg, 0.33 mmol) and benzyl (2S,4R)-4-fluoro-4-methylpyrrolidine-2-carboxylate hydrochloride (137 mg, 0.50 mmol) in DMF (3 mL) was added T₃P (630 mg, 0.99 mmol, 50% wt. in EtOAc) and DIPEA (0.33 mL, 1.98 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-40% EtOAc in PE) to give benzyl (2S,4R)-1-((9,9-difluoro-9H-fluorene-3-carbonyl)glycyl)-4-fluoro-4-methylpyrrolidine-2-carboxylate (48 mg, yield 27.9%) as a yellow oil. LCMS (ESI) m/z: 523 (M+H)⁺.

### Step 5: (2S,4R)-1-((9,9-difluoro-9H-fluorene-3-carbonyl)glycyl)-4-fluoro-4-methylpyrrolidine-2-carboxylic acid

A solution of benzyl (2S,4R)-1-((9,9-difluoro-9H-fluorene-3-carbonyl)glycyl)-4-fluoro-4-methylpyrrolidine-2-carboxylate (48 mg, 0.09 mmol) in MeOH (2.0 mL) and water (0.6 mL) was added LiOH·H₂O (7.7 mg, 0.18 mmol) at 0° C. and the mixture was stirred at room temperature for 1 hour. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (2S,4R)-1-((9,9-difluoro-9H-fluorene-3-carbonyl)glycyl)-4-fluoro-4-methylpyrrolidine-2-carboxylic acid (27 mg, yield 67.5%) as a yellow solid, which was used directly in the next reaction. LCMS (ESI) (m/z): 433 (M+H)⁺.

### Step 6: (2S,4R)—N—((R)-1-(4-carbamimidoylthiophen-2-yl)ethyl)-1-((9,9-difluoro-9H-fluorene-3-carbonyl)glycyl)-4-fluoro-4-methylpyrrolidine-2-carboxamide (Compound 266)

To a mixture of (2S,4R)-1-((9,9-difluoro-9H-fluorene-3-carbonyl)glycyl)-4-fluoro-4-methylpyrrolidine-2-carboxylic acid (27 mg, 0.06 mmol) and (R)-5-(1-aminoethyl)thiophene-3-carboximidamide (39 mg, 0.18 mmol) in DMF (3.0 mL) was added DIPEA (73 mg, 0.54 mmol) and PyBOP (48 mg, 0.09 mmol) at 0° C. and the mixture was stirred under N₂ atmosphere at room temperature for 2 hours. The mixture was concentrated to dryness under reduced pressure. The residue was purified by prep-TLC (DCM:MeOH=10:1) and further purified by prep-HPLC to give Compound 268 (3.0 mg, yield 8.3%) as a white solid. ¹H NMR (400 MHz, CD₃OD) δ 8.53 (s, 1H), 8.20 (d, J=8.8 Hz, 2H), 7.89 (d, J=7.8 Hz, 1H), 7.77 (d, J=7.5 Hz, 1H), 7.72 (d, J=7.6 Hz, 1H), 7.66 (d, J=7.3 Hz, 1H), 7.57 (d, J=13.0 Hz, 2H), 7.45 (t, J=7.6 Hz, 1H), 5.32-5.24 (m, 1H), 4.63-4.58 (m, 1H), 4.35 (d, J=16.9 Hz, 1H), 4.16 (d, J=16.7 Hz, 1H), 4.11-4.02 (m, 1H), 3.84-3.73 (m, 1H), 2.69-2.52 (m, 1H), 2.11-2.03 (m, 1H), 1.60 (dd, J=13.6, 11.4 Hz, 6H). LCMS (ESI) (m/z): 584 (M+H)⁺.

### Scheme 280: Synthesis of (2S,4S)-4-(((2-acetamidoethyl)amino)methyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (Compound 269)

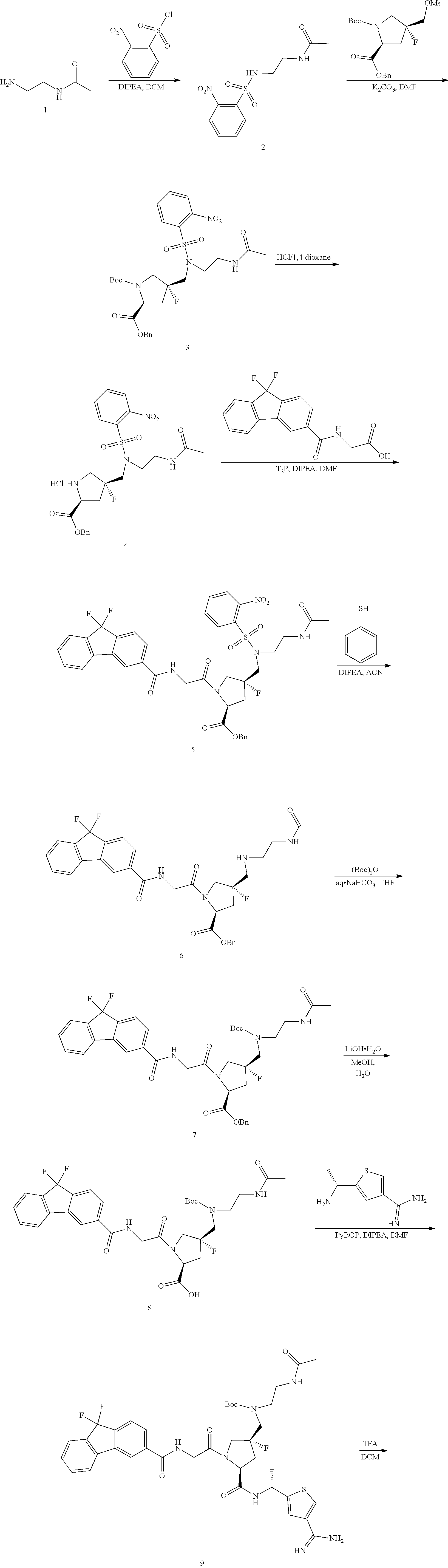

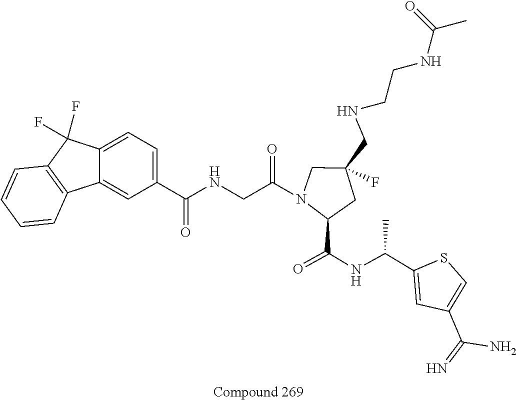 [SMILES: CC(=O)NCCNC[C@@]1(F)C[C@@H](C(=O)N[C@H](C)c2cc(C(=N)N)cs2)N(C(=O)CNC(=O)c2ccc3c(c2)-c2ccccc2C3(F)F)C1]

### Step 1: N-(2-((2-nitrophenyl)sulfonamido)ethyl)acetamide

To a mixture of N-(2-aminoethyl)acetamide (1.0 g, 9.80 mmol) and 2-nitrobenzenesulfonyl chloride (2.2 g, 9.80 mmol) in DCM (15 mL) was added DIPEA (4.85 mL, 29.4 mmol) under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give N-(2-((2-nitrophenyl)sulfonamido) ethyl)acetamide (2.18 g, yield 77.6%) as a light-yellow solid. LC/MS (ESI) m/z: 288 (M+H)⁺.

### Step 2: 2-benzyl 1-(tert-butyl) (2S,4R)-4-(((N-(2-acetamidoethyl)-2-nitrophenyl)sulfonamido) methyl)-4-fluoropyrrolidine-1,2-dicarboxylate

To a mixture of N-(2-((2-nitrophenyl)sulfonamido)ethyl)acetamide (163 mg, 0.57 mmol) and 2-benzyl 1-(tert-butyl) (2S,4R)-4-fluoro-4-(((methylsulfonyl)oxy)methyl)pyrrolidine-1,2-dicarboxylate (164 mg, 0.38 mmol) in DMF (3 mL) was added K₂CO₃ (262 mg, 1.90 mmol) under N₂ atmosphere and the reaction mixture was stirred at 100° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give 2-benzyl 1-(tert-butyl) (2S,4R)-4-(((N-(2-acetamidoethyl)-2-nitrophenyl)sulfonamido)methyl)-4-fluoropyrrolidine-1,2-dicarboxylate (70 mg, yield 29.7%) as a yellow solid. LC/MS (ESI) m/z: 623 (M+H)⁺.

### Step 3: benzyl (2S,4R)-4-(((N-(2-acetamidoethyl)-2-nitrophenyl)sulfonamido) methyl)-4-fluoropyrrolidine-2-carboxylate hydrochloride

A solution of 2-benzyl 1-(tert-butyl) (2S,4R)-4-(((N-(2-acetamidoethyl)-2-nitrophenyl)sulfonamido)methyl)-4-fluoropyrrolidine-1,2-dicarboxylate (70 mg, 0.11 mmol) in HCl/1,4-dioxane (3 mL, 4M) was stirred at room temperature for 1 hour. The mixture was concentrated to dryness under reduced pressure to give benzyl (2S,4R)-4-(((N-(2-acetamidoethyl)-2-nitrophenyl)sulfonamido)methyl)-4-fluoropyrrolidine-2-carboxylate hydrochloride (61 mg, yield 98.9%) as a yellow oil, which was directly used in the next reaction without further purification. LC/MS (ESI) m/z: 523 (M+H)⁺.

### Step 4: benzyl (2S,4R)-4-(((N-(2-acetamidoethyl)-2-nitrophenyl)sulfonamido) methyl)-1-((9,9-difluoro-9H-fluorene-3-carbonyl)glycyl)-4-fluoropyrrolidine-2-carboxylate

To a mixture of (9,9-difluoro-9H-fluorene-3-carbonyl)glycine (34 mg, 0.11 mmol) and benzyl (2S,4R)-4-(((N-(2-acetamidoethyl)-2- nitrophenyl)sulfonamido)methyl)-4-fluoropyrrolidine-2-carboxylate hydrochloride (58 mg, 0.11 mmol) in DMF (3 mL) was added T₃P (212 mg, 0.33 mmol, 50% wt. in EtOAc) and DIPEA (0.11 mL, 0.66 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give benzyl (2S,4R)-4-(((N-(2-acetamidoethyl)-2-nitrophenyl) sulfonamido)methyl)-1-((9,9-difluoro-9H-fluorene-3-carbonyl)glycyl)-4-fluoropyrrolidine-2-carboxylate (40 mg, yield 44.5%) as a yellow oil. LCMS (ESI) m/z: 808 (M+H)⁺.

### Step 5: benzyl (2S,4S)-4-(((2-acetamidoethyl)amino)methyl)-1-((9,9-difluoro-9H-fluorene-3-carbonyl)glycyl)-4-fluoropyrrolidine-2-carboxylate

To a mixture of benzyl (2S,4R)-4-(((N-(2-acetamidoethyl)-2-nitrophenyl)sulfonamido) methyl)-1-((9,9-difluoro-9H-fluorene-3-carbonyl)glycyl)-4-fluoropyrrolidine-2-carboxylate (40 mg, 0.05 mmol) and benzenethiol (11 mg, 0.10 mmol) in acetonitrile (2 mL) was added DIPEA (38 mg, 0.30 mmol) under N₂ atmosphere and the reaction mixture was stirred at room temperature for 16 hours. The mixture was diluted with EtOAc, washed with water and brine, dried over anhydrous Na₂SO₄, filtered, and concentrated under reduced pressure to dryness. The residue was purified by prep-TLC (DCM:MeOH=20:1) to give benzyl (2S,4S)-4-(((2-acetamidoethyl)amino)methyl)-1-((9,9-difluoro-9H-fluorene-3-carbonyl)glycyl)-4-fluoropyrrolidine-2-carboxylate (23 mg, yield 76.7%) as a colorless oil. LCMS (ESI) m/z: 623 (M+H)⁺.

### Step 6: benzyl (2S,4R)-4-(((2-acetamidoethyl)(tert-butoxycarbonyl)amino)methyl)-1-((9,9-difluoro-9H-fluorene-3-carbonyl)glycyl)-4-fluoropyrrolidine-2-carboxylate

To a mixture of benzyl (2S,4S)-4-(((2-acetamidoethyl)amino)methyl)-1-((9,9-difluoro-9H-fluorene-3-carbonyl)glycyl)-4-fluoropyrrolidine-2-carboxylate (23 mg, 0.04 mmol) and NaHCO₃ (16 mg, 0.20 mmol) in THE (1 mL) and H₂O (1 mL) was added (Boc)₂O (12 mg, 0.06 mmol) at 0° C. and the reaction mixture was stirred at room temperature for 16 hours. The mixture was diluted with EtOAc, washed with brine, dried over anhydrous Na₂SO₄, filtered, and concentrated under reduced pressure to dryness. The residue was purified by prep-TLC (DCM:MeOH=20:1) to give benzyl (2S,4R)-4-(((2-acetamidoethyl)(tert-butoxycarbonyl) amino)methyl)-1-((9,9-difluoro-9H-fluorene-3-carbonyl)glycyl)-4-fluoropyrrolidine-2-carboxylate (25 mg, yield 92.6%) as a colorless oil. LC/MS (ESI) m/z: 723 (M+H)⁺.

### Step 7: (2S,4R)-4-(((2-acetamidoethyl)(tert-butoxycarbonyl)amino)methyl)-1-((9,9-difluoro-9H-fluorene-3-carbonyl)glycyl)-4-fluoropyrrolidine-2-carboxylic acid

To a solution of benzyl (2S,4R)-4-(((2-acetamidoethyl)(tert-butoxycarbonyl) amino)methyl)-1-((9,9-difluoro-9H-fluorene-3-carbonyl)glycyl)-4-fluoropyrrolidine-2-carboxylate (30 mg, 0.04 mmol) in MeOH (2 mL) and water (0.4 mL) was added LiOH·H₂O (3.5 mg, 0.08 mmol) at 0° C. and the mixture was stirred at room temperature for 1 hour. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (2S,4R)-4-(((2-acetamidoethyl)(tert-butoxycarbonyl)amino)methyl)-1-((9,9-difluoro-9H-fluorene-3-carbonyl)glycyl)-4-fluoropyrrolidine-2-carboxylic acid (18 mg, yield 69.2%) as a white solid, which was used directly in the next reaction. LCMS (ESI) (m/z): 633 (M+H)⁺.

### Step 8: tert-butyl (2-acetamidoethyl)(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methyl)carbamate

To a mixture of (2S,4R)-4-(((2-acetamidoethyl)(tert-butoxycarbonyl)amino)methyl)-1-((9,9-difluoro-9H-fluorene-3-carbonyl)glycyl)-4-fluoropyrrolidine-2-carboxylic acid (18 mg, 0.03 mmol) and (R)-5-(1-aminoethyl)thiophene-3-carboximidamide (14 mg, 0.09 mmol) in DMF (2.0 mL) was added DIPEA (33 mg, 0.27 mmol) and PyBOP (15 mg, 0.03 mmol) at 0° C. and the mixture was stirred under N₂ atmosphere at room temperature for 2 hours. The mixture was concentrated to dryness under reduced pressure. The residue was purified by prep-TLC (DCM:MeOH=10:1) to give tert-butyl (2-acetamidoethyl)(((3R,5S)-5-(((R)-1-(4-carbamimidoylthiophen-2-yl)ethyl) carbamoyl)-1-((9,9-difluoro-9H-fluorene-3-carbonyl)glycyl)-3-fluoropyrrolidin-3-yl)methyl)carbamate (20 mg, yield 90.9%) as a white solid. LCMS (ESI) (m/z): 784 (M+H)⁺.

### Step 9: (2S,4S)-4-(((2-acetamidoethyl)amino)methyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (Compound 269)

To a solution of tert-butyl (2-acetamidoethyl)(((3R,5S)-5-(((R)-1-(4-carbamimidoylthiophen-2-yl)ethyl)carbamoyl)-1-((9,9-difluoro-9H-fluorene-3-carbonyl)glycyl)-3-fluoropyrrolidin-3-yl)methyl)carbamate (20 mg, 0.03 mmol) in DCM (1 mL) was added TFA (0.5 mL) and the mixture was stirred at room temperature for 1 hour. The mixture was concentrated to dryness under reduced pressure. The residue was purified by prep-HPLC to give Compound 269 (1.2 mg, yield 7.1%) as a white solid. ¹H NMR (400 MHz, CD₃OD) δ 8.45 (s, 1H), 8.21 (d, J=9.4 Hz, 2H), 7.89 (d, J=8.1 Hz, 1H), 7.77 (d, J=7.4 Hz, 1H), 7.73 (d, J=7.9 Hz, 1H), 7.66 (d, J=9.3 Hz, 1H), 7.59-7.54 (m, 2H), 7.46 (d, J=7.0 Hz, 1H), 5.17 (s, 1H), 4.62 (s, 1H), 4.38-4.13 (m, 2H), 3.88 (dd, J=33.2, 14.4 Hz, 2H), 3.48 (d, J=1.9 Hz, 2H), 3.45 (s, 1H), 3.15-2.98 (m, 3H), 2.79 (t, J=6.7 Hz, 2H), 1.96 (s, 3H), 1.61-1.57 (m, 3H). LCMS (ESI) (m/z): 684 (M+H)⁺.

### Scheme 281: Synthesis of (S)—N—((R)-1-(4-carbamimidoylthiophen-2-yl)ethyl)-1-((9,9-difluoro-9H-fluorene-3-carbonyl)glycyl)-4-(difluoromethylene)pyrrolidine-2-carboxamide (Compound 270)

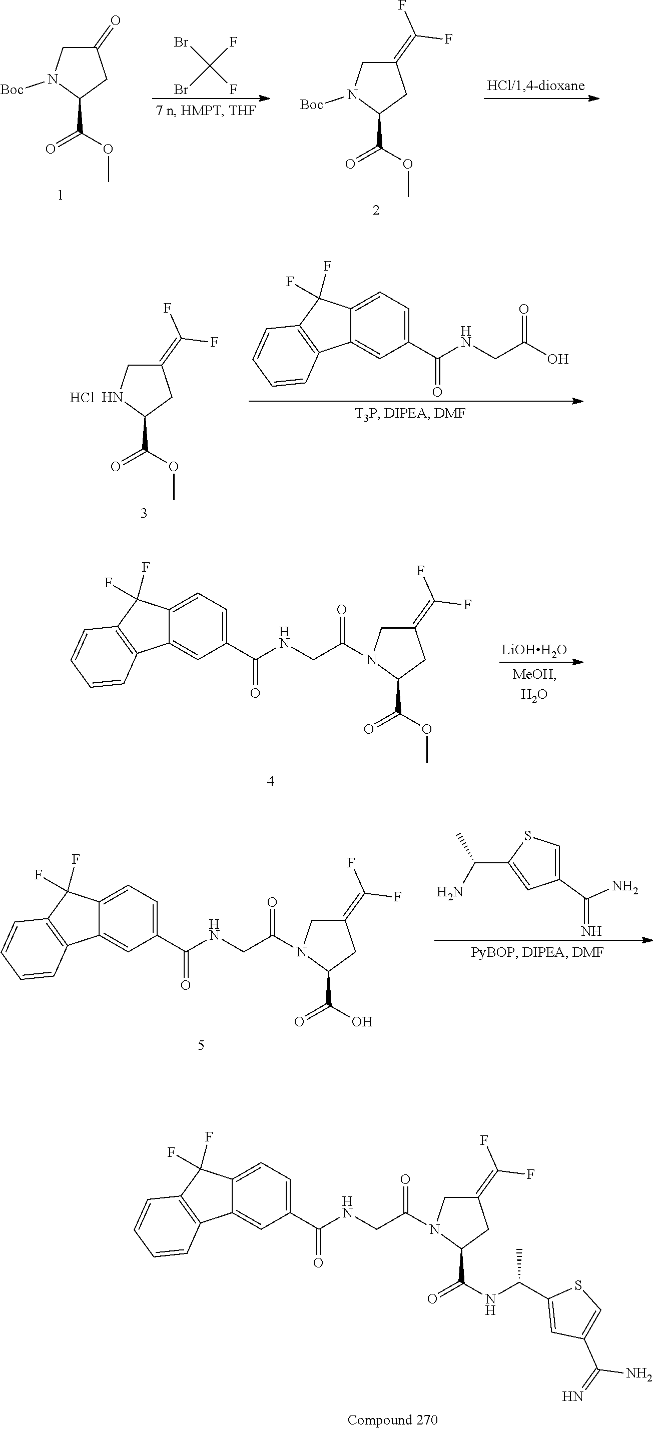

### Step 1: 1-(tert-butyl) 2-methyl (S)-4-(difluoromethylene)pyrrolidine-1,2-dicarboxylate

To a mixture of 1-(tert-butyl) 2-methyl (S)-4-oxopyrrolidine-1,2-dicarboxylate (1.0 g, 4.12 mmol) and dibromodifluoromethane (889 mg, 4.12 mmol) in THE (5 mL) was added HMPT (671 mg, 4.12 mmol) and Zn (267 mg, 4.12 mmol) at 0° C. and the reaction mixture was stirred at 70° C. for 16 hours in a sealed tube. The mixture was diluted with EtOAc, washed with water and brine, dried over anhydrous Na₂SO₄, filtered, and concentrated under reduced pressure to dryness. The residue was purified by flash chromatography (silica gel, 0-5% EtOAc in PE) to give 1-(tert-butyl) 2-methyl (S)-4-(difluoromethylene) pyrrolidine-1,2-dicarboxylate (142 mg, yield 12.5%) as a yellow oil. LC/MS (ESI) m/z: 278 (M+H)⁺.

### Step 2: methyl (S)-4-(difluoromethylene)pyrrolidine-2-carboxylate hydrochloride

A solution of 1-(tert-butyl) 2-methyl (S)-4-(difluoromethylene)pyrrolidine-1,2-dicarboxylate (142 mg, 0.51 mmol) in HCl/1,4-dioxane (3 mL, 4M) was stirred at room temperature for 1 hour. The mixture was concentrated under reduced pressure to dryness to give methyl (S)-4-(difluoromethylene)pyrrolidine-2-carboxylate hydrochloride (90 mg, yield 98.9%) as a yellow oil, which was directly used in the next reaction without further purification. LC/MS (ESI) m/z: 178 (M+H)⁺.

### Step 3: benzyl (2S,4R)-1-((9,9-difluoro-9H-fluorene-3-carbonyl)glycyl)-4-fluoro-4-methylpyrrolidine-2-carboxylate

To a mixture of (9,9-difluoro-9H-fluorene-3-carbonyl)glycine (154 mg, 0.51 mmol) and methyl (S)-4-(difluoromethylene)pyrrolidine-2-carboxylate hydrochloride (90 mg, 0.51 mmol) in DMF (3 mL) was added T₃P (970 mg, 1.53 mmol, 50% wt. in EtOAc) and DIPEA (0.50 mL, 3.06 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-40% EtOAc in PE) to give methyl (S)-1-((9,9-difluoro-9H-fluorene-3-carbonyl) glycyl)-4-(difluoromethylene)pyrrolidine-2-carboxylate (126 mg, yield 53.6%) as a yellow oil. LCMS (ESI) m/z: 463 (M+H)⁺.

### Step 4: (S)-1-((9,9-difluoro-9H-fluorene-3-carbonyl)glycyl)-4-(difluoromethylene) pyrrolidine-2-carboxylic acid

To a solution of methyl (S)-1-((9,9-difluoro-9H-fluorene-3-carbonyl)glycyl)-4-(difluoromethylene)pyrrolidine-2-carboxylate (126 mg, 0.27 mmol) in MeOH (3.0 mL) and water (1.0 mL) was added LiOH·H₂O (17 mg, 0.68 mmol) at 0° C. and the mixture was stirred at room temperature for 1 hour. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (S)-1-((9,9-difluoro-9H-fluorene-3-carbonyl)glycyl)-4-(difluoromethylene)pyrrolidine-2-carboxylic acid (100 mg, yield 82.0%) as a yellow oil, which was used directly in the next reaction. LCMS (ESI) (m/z): 449 (M+H)⁺.

### Step 5: (S)—N—((R)-1-(4-carbamimidoylthiophen-2-yl)ethyl)-1-((9,9-difluoro-9H-fluorene-3-carbonyl)glycyl)-4-(difluoromethylene)pyrrolidine-2-carboxamide (Compound 270)

To a mixture of (S)-1-((9,9-difluoro-9H-fluorene-3-carbonyl)glycyl)-4-(difluoromethylene)pyrrolidine-2-carboxylic acid (80 mg, 0.19 mmol) and (R)-5-(1-aminoethyl)thiophene-3-carboximidamide (91 mg, 0.57 mmol) in DMF (3.0 mL) was added DIPEA (141 mg, 1.14 mmol) and PyBOP (111 mg, 0.23 mmol) at 0° C. and the mixture was stirred under N₂ atmosphere at room temperature for 2 hours. The mixture was concentrated to dryness under reduced pressure. The residue was purified by prep-TLC (DCM:MeOH=10:1) and further purified by prep-HPLC to give Compound 270 (10 mg, yield 9.3%) as a white solid. ¹H NMR (400 MHz, CD₃OD) δ 8.52 (s, 1H), 8.22 (t, J=12.2 Hz, 2H), 7.88 (t, J=7.6 Hz, 1H), 7.74 (dd, J=16.9, 7.8 Hz, 2H), 7.66 (d, J=7.1 Hz, 1H), 7.58 (t, J=7.4 Hz, 1H), 7.54-7.41 (m, 2H), 5.28 (dd, J=13.3, 6.4 Hz, 1H), 4.75 (d, J=9.0 Hz, 1H), 4.48 (s, 2H), 4.26 (dt, J=29.6, 14.8 Hz, 2H), 3.15-2.95 (m, 1H), 2.72 (d, J=15.9 Hz, 1H), 1.59 (d, J=6.9 Hz, 3H). LCMS (ESI) (m/z): 600 (M+H)⁺.

### Scheme 282: (S)—N—((R)-1-(4-carbamimidoylthiophen-2-yl)ethyl)-7-((phenoxathiine-3-carbonyl)glycyl)-1,4-dioxa-7-azaspiro[4.4]nonane-8-carboxamide (Compound 271)

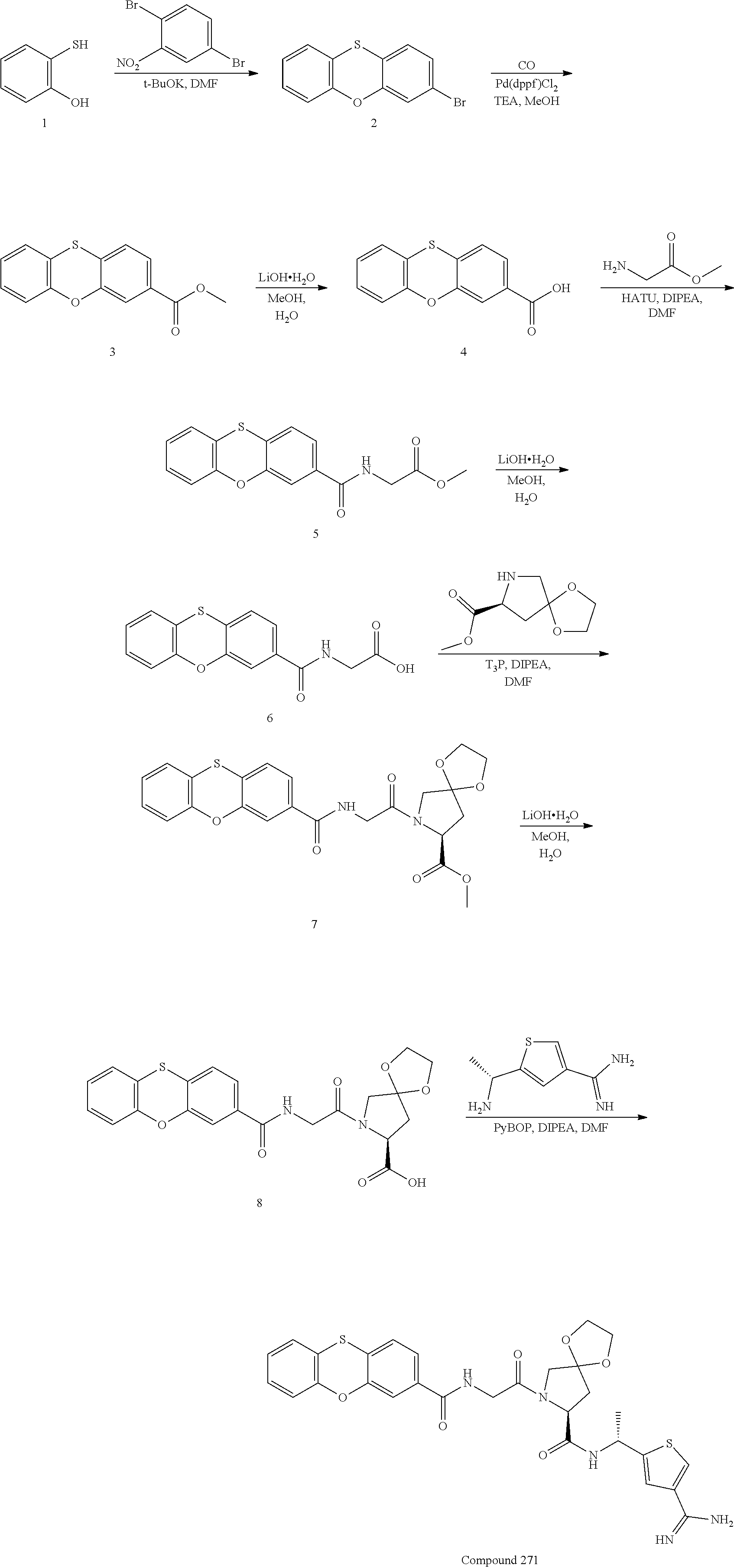

### Step 1: 3-bromophenoxathiine (2)

To a solution of 2-mercaptophenol (3.0 g, 23.81 mmol) in DMF (71 mL) was added 1,4-dibromo-2-nitrobenzene (6.64 g, 23.81 mmol) and t-BuOK (5.33 g, 47.62 mmol) at 0° C. and the mixture was stirred at 160° C. for 8 hours. The mixture was diluted with EtOAc and washed with water twice. The combined organic layers were dried over anhydrous Na₂SO₄, filtered, and concentrated to dryness under reduced pressure. The residue was purified by flash chromatography (silica gel, 100% PE) to give 3-bromophenoxathiine (4.24 g, yield 64.1%) as a white solid. ¹H NMR (400 MHz, CDCl₃) δ 7.16 (d, J=0.7 Hz, 1H), 7.12 (d, J=7.7 Hz, 2H), 7.08 (d, J=7.6 Hz, 1H), 7.03 (d, J=7.4 Hz, 1H), 6.98 (d, J=8.1 Hz, 1H), 6.94 (d, J=8.2 Hz, 1H).

### Step 2: methyl phenoxathiine-3-carboxylate (3)

To a solution of 3-bromophenoxathiine (1.0 g, 3.6 mmol) in MeOH (10 mL) was added TEA (1.5 mL, 10.8 mmol) and Pd(dppt)Cl₂ (526 mg, 0.72 mmol) and the mixture was stirred under CO atmosphere at 85° C. for 16 hours. The mixture was diluted with water and extracted with DCM twice. The combined organic layers were dried over anhydrous Na₂SO₄, filtered, and concentrated under reduced pressure to dryness. The residue was purified by flash chromatography (silica gel, 0-8% EtOAc in PE) to give methyl phenoxathiine-3-carboxylate (647 mg, yield 69.7%) as a white solid. ¹H NMR (400 MHz, CDCl₃) δ 8.09 (d, J=8.1 Hz, 1H), 8.05 (s, 1H), 7.58-7.53 (m, 2H), 7.50 (d, J=7.2 Hz, 1H), 7.44 (dd, J=10.7, 8.2 Hz, 2H).

### Step 3: phenoxathiine-3-carboxylic acid (4)

To a solution of methyl phenoxathiine-3-carboxylate (300 mg, 1.16 mmol) in MeOH (2 mL)/THF (1 mL)/H₂O (1 mL) was added LiOH·H₂O (146 mg, 3.48 mmol) at 0° C. and the mixture was stirred at room temperature for 1 hour. The mixture was acidified with 1 N aq·HCl to pH ˜3 and extracted with EtOAc twice. The combined organic layers were washed with brine, dried over anhydrous Na₂SO₄, filtered, and concentrated under reduced pressure to dryness to give phenoxathiine-3-carboxylic acid (280 mg, yield 98.7%) as a yellow solid, which was used directly in the next step without purification. LCMS (ESI) (m/z): 245 (M+H)⁺.

### Step 4: methyl (phenoxathiine-3-carbonyl)glycinate (5)

To a mixture of phenoxathiine-3-carboxylic acid (329 mg, 1.35 mmol) and methyl glycinate (338 mg, 2.69 mmol) in DMF (5.0 mL) was added DIPEA (1.04 g, 8.09 mmol) and HATU (615 mg, 1.62 mmol) at 0° C. under N₂ atmosphere and the mixture was stirred at 25° C. for 2 hours. The mixture was diluted with EtOAc and washed with water twice. The combined organic layers were dried over anhydrous Na₂SO₄, filtered, and concentrated under reduced pressure to dryness. The residue was purified by flash chromatography (silica gel, 0-13% EtOAc in PE) to give methyl (phenoxathiine-3-carbonyl)glycinate (370 mg, yield 87.1%) as a yellow solid. LC/MS (ESI) m/z: 316 (M+H)⁺.

### Step 5: (phenoxathiine-3-carbonyl)glycine (6)

To a solution of methyl (phenoxathiine-3-carbonyl)glycinate (370 mg, 1.17 mmol) in MeOH (2 mL)/THF (1 mL)/H₂O (1 mL) was added LiOH·H₂O (148 mg, 3.51 mmol) at 0° C. and the mixture was stirred at room temperature for 1 hour. The mixture was acidified with 1 N aq·HCl to pH ˜3 and extracted with EtOAc twice. The combined organic layers were washed with brine, dried over anhydrous Na₂SO₄, filtered, and concentrated under reduced pressure to dryness to give (phenoxathiine-3-carbonyl)glycine (345 mg, yield 97.6%) as a yellow solid, which was used directly in the next step. LCMS (ESI) (m/z): 302 (M+H)⁺.

### Step 6: methyl (S)-7-((phenoxathiine-3-carbonyl)glycyl)-1,4-dioxa-7-azaspiro[4.4]nonane-8-carboxylate (7)

To a mixture of (phenoxathiine-3-carbonyl)glycine (160 mg, 0.53 mmol) and methyl (S)-1,4-dioxa-7-azaspiro[4.4]nonane-8-carboxylate (150 mg, 0.8 mmol) in DMF (3.0 mL) was added DIPEA (411 mg, 3.20 mmol) and T₃P (1.02 g, 3.20 mmol) at 0° C. under N₂ atmosphere and the mixture was stirred at 25° C. for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-5% EtOAc in PE) to give methyl (S)-7-((phenoxathiine-3-carbonyl)glycyl)-1,4-dioxa-7-azaspiro[4.4]nonane-8-carboxylate (200 mg, yield 80.1%) as a white solid. LC/MS (ESI) m/z: 471 (M+H)⁺.

### Step 7: (S)-7-((phenoxathiine-3-carbonyl)glycyl)-1,4-dioxa-7-azaspiro[4.4]nonane-8-carboxylic acid (8)

To a solution of methyl (S)-7-((phenoxathiine-3-carbonyl)glycyl)-1,4-dioxa-7-azaspiro[4.4]nonane-8-carboxylate (200 mg, 0.43 mmol) in MeOH (3 mL) and H₂O (1 mL) was added LiOH·H₂O (53.6 mg, 1.29 mmol) at 0° C. and the mixture was stirred at room temperature for 1 hour. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (S)-7-((phenoxathiine-3-carbonyl)glycyl)-1,4-dioxa-7-azaspiro[4.4]nonane-8-carboxylic acid (190 mg, yield 97.9%) as a yellow solid, which was used directly in the next step. LCMS (ESI) (m/z): 457 (M+H)⁺.

### Step 8: (S)—N—((R)-1-(4-carbamimidoylthiophen-2-yl)ethyl)-7-((phenoxathiine-3-carbonyl)glycyl)-1,4-dioxa-7-azaspiro[4.4]nonane-8-carboxamide (Compound 271)

To a mixture of (S)-7-((phenoxathiine-3-carbonyl)glycyl)-1,4-dioxa-7-azaspiro [4.4]nonane-8-carboxylic acid (80 mg, 0.18 mmol) and (R)-5-(1-aminoethyl)thiophene-3-carboximidamide (60 mg, 0.36 mmol) in DMF (3 mL) was added DIPEA (135 mg, 1.08 mmol) and PyBOP (91.2 mg, 0.18 mmol) at 0° C. and the mixture was stirred under N₂ atmosphere at room temperature for 1 hour. The mixture was concentrated under reduced pressure to dryness. The residue was purified by prep-TLC (DCM:MeOH=10:1) and further purified by prep-HPLC to give Compound 271 (30 mg, yield 28.2%) as a white solid. ¹H NMR (400 MHz, CD₃OD) δ 8.11 (s, 1H), 7.53 (d, J=8.1 Hz, 1H), 7.49 (s, 2H), 7.23 (d, J=8.2 Hz, 1H), 7.19 (d, J=8.2 Hz, 1H), 7.15 (d, J=6.9 Hz, 1H), 7.08 (d, J=7.6 Hz, 1H), 7.04 (d, J=8.1 Hz, 1H), 5.27 (d, J=7.0 Hz, 1H), 4.58-4.54 (m, 1H), 4.14 (d, J=4.4 Hz, 2H), 4.02-3.97 (m, 4H), 3.76 (t, J=7.3 Hz, 2H), 2.42 (dd, J=12.9, 8.9 Hz, 1H), 2.21 (dd, J=13.1, 6.6 Hz, 1H), 1.57 (d, J=7.0 Hz, 3H). LCMS (ESI) (m/z): 608 (M+H)⁺.

### Scheme 283: Synthesis of (S)—N—((R)-1-(4-carbamimidoylthiophen-2-yl)ethyl)-7-((4-hydroxybenzoyl)glycyl)-1,4-dioxa-7-azaspiro[4.4]nonane-8-carboxamide (Compound 272)

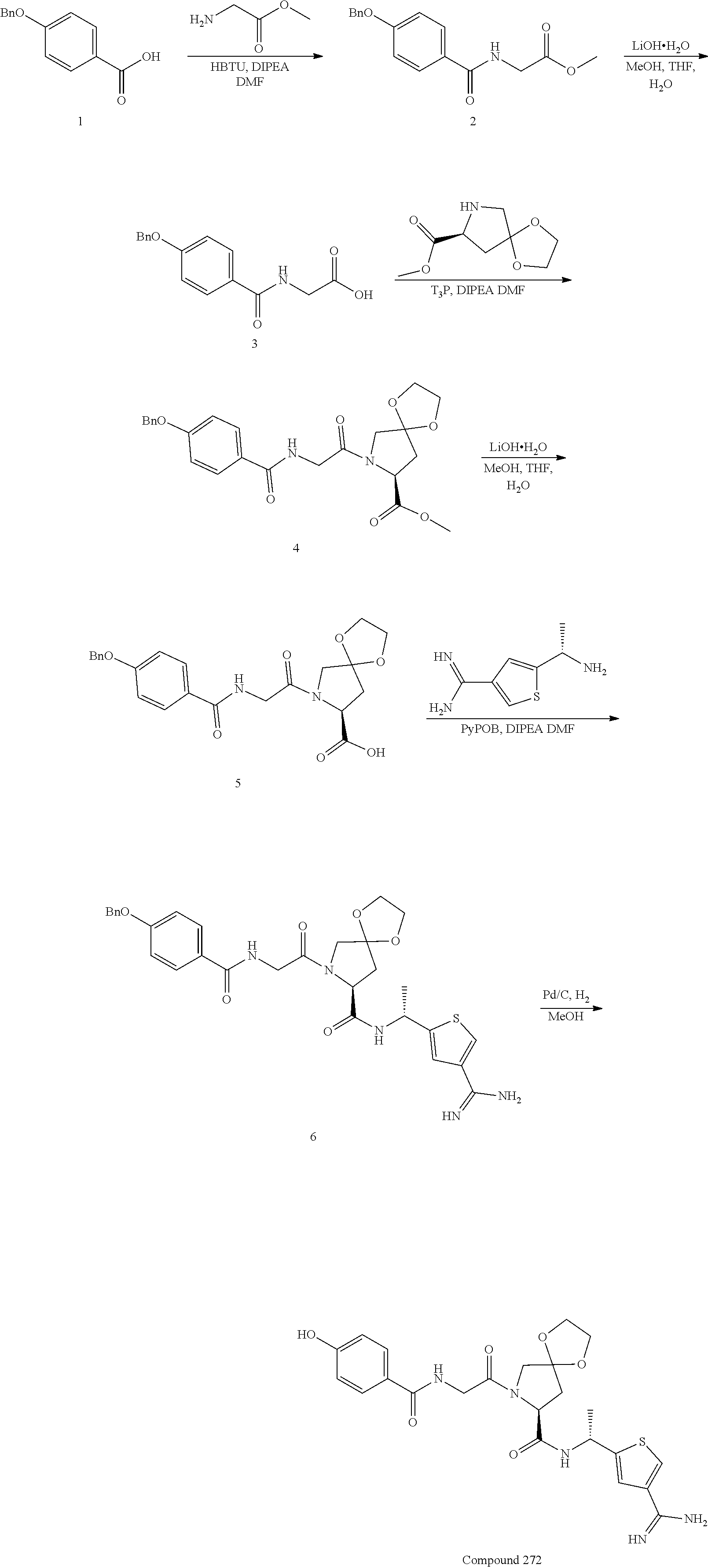

### Step 1: methyl (4-(benzyloxy)benzoyl)glycinate

To a mixture of 4-(benzyloxy)benzoic acid (500 mg, 2.19 mmol) and methyl glycinate (234 mg, 2.63 mmol) in DMF (8 mL) was added DIPEA (1.4 g, 10.95 mmol) and HBTU (1.2 g, 3.29 mmol) under N₂ atmosphere and the reaction mixture was stirred at room temperature for 16 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give methyl (4-(benzyloxy)benzoyl)glycinate (600 mg, yield 91.5%) as a yellow oil. LCMS (ESI) m/z: 300 (M+H)⁺.

### Step 2: (4-(benzyloxy)benzoyl)glycine

To a solution of methyl (4-(benzyloxy)benzoyl)glycinate (590 mg, 1.97 mmol) in MeOH (6 mL), THE (3 mL) and water (3 mL) was added LiOH·H₂O (248 mg, 5.91 mmol) at 0° C. and the mixture was stirred at room temperature for 2 hours. The mixture was acidified with 1 N aq·HCl solution to pH ˜3 and extracted with EtOAc twice. The combined organic layers were washed with brine, dried over anhydrous Na₂SO₄, filtered, and concentrated to dryness to give (4-(benzyloxy)benzoyl)glycine (500 mg, yield 88.9%) as a white solid, which was used directly in next reaction without further purification. LC/MS (ESI) (m/z): 286 (M−H)⁻.

### Step 3: methyl (S)-7-((4-(benzyloxy)benzoyl)glycyl)-1,4-dioxa-7-azaspiro[4.4]nonane-8-carboxylate

To a mixture of (4-(benzyloxy)benzoyl)glycine (100 mg, 0.35 mmol) and methyl (S)-1,4-dioxa-7-azaspiro[4.4]nonane-8-carboxylate (79 mg, 0.42 mmol) in DMF (3 mL) was added DIPEA (226 mg, 1.75 mmol) and T₃P (335 mg, 0.53 mmol, 50% wt. in EtOAc) under N₂ atmosphere and the mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-50% EtOAc in PE) to give methyl (S)-7-((4-(benzyloxy)benzoyl)glycyl)-1,4-dioxa-7-azaspiro[4.4]nonane-8-carboxylate (150 mg, yield 94.2%) as a colorless oil. LC/MS (ESI) (m/z): 455 (M+H)⁺.

### Step 4: (S)-7-((4-(benzyloxy)benzoyl)glycyl)-1,4-dioxa-7-azaspiro[4.4]nonane-8-carboxylic acid

To a solution of methyl (S)-7-((4-(benzyloxy)benzoyl)glycyl)-1,4-dioxa-7-azaspiro[4.4]nonane-8-carboxylate (150 mg, 0.33 mmol) in MeOH (2 mL), THF (1 mL) and water (1 mL) was added LiOH·H₂O (42 mg, 0.99 mmol) at 0° C. and the mixture was stirred at room temperature for 1 hour. The mixture was acidified with 1 N aq·HCl solution to pH ˜3 and extracted with EtOAc twice. The combined organic layers were washed with brine, dried over anhydrous Na₂SO₄, filtered, and concentrated under reduced pressure to dryness to give (S)-7-((4-(benzyloxy)benzoyl)glycyl)-1,4-dioxa-7-azaspiro[4.4]nonane-8-carboxylic acid (132 mg, yield 90.8%) as a white solid, which was used directly in next reaction without further purification. LC/MS (ESI) (m/z): 441 (M+H)⁺.

### Step 5: (S)-7-((4-(benzyloxy)benzoyl)glycyl)-N—((R)-1-(4-carbamimidoylthiophen-2-yl)ethyl)-1,4-dioxa-7-azaspiro[4.4]nonane-8-carboxamide

To a solution of (S)-7-((4-(benzyloxy)benzoyl)glycyl)-1,4-dioxa-7-azaspiro[4.4]nonane-8-carboxylic acid (60 mg, 0.14 mmol) and (S)-5-(1-aminoethyl)thiophene-3-carboximidamide (46 mg, 0.27 mmol) in DMF (3 mL) was added DIPEA (88 mg, 0.68 mmol) and PyBOP (106 mg, 0.20 mmol) at 0° C. under N₂ atmosphere and the mixture was stirred at 25° C. for 1 hour. The reaction mixture was diluted with water and extracted with EtOAc twice. The combined organic layers were washed with brine, dried over anhydrous Na₂SO₄, filtered, and concentrated under reduced pressure to dryness. The residue was purified by prep-TLC (DCM:MeOH=20:1) to give (S)-7-((4-(benzyloxy)benzoyl)glycyl)-N—((R)-1-(4-carbamimidoylthiophen-2-yl)ethyl)-1,4-dioxa-7-azaspiro[4.4]nonane-8-carboxamide (40 mg, yield 49.6%) as a colorless oil. LCMS (ESI) (m/z): 592 (M+H)⁺.

### Step 6: (S)—N—((R)-1-(4-carbamimidoylthiophen-2-yl)ethyl)-7-((4-hydroxybenzoyl) glycyl)-1,4-dioxa-7-azaspiro[4.4]nonane-6-carboxamide (Compound 272)

To a mixture of (S)-7-((4-(benzyloxy)benzoyl)glycyl)-N—((R)-1-(4-carbamimidoylthiophen-2-yl)ethyl)-1,4-dioxa-7-azaspiro[4.4]nonane-8-carboxamide (20 mg, 0.03 mmol) in MeOH (2 mL) was added Pd/C (5 mg, 10% wt.), the mixture was degassed under N₂ atmosphere for three times and stirred under a H₂ balloon at 25° C. for 0.5 hours. The mixture was filtered, and the filtrate was concentrated under reduced pressure to dryness. The residue was purified by prep-HPLC to give Compound 272 (1.7 mg, yield 10.0%) as a white solid. ¹H NMR (400 MHz, CD₃OD) δ 8.53 (s, 1H), 8.24 (d, J=6.1 Hz, 1H), 7.73 (d, J=8.7 Hz, 2H), 7.52 (s, 1H), 6.83 (d, J=8.7 Hz, 2H), 5.27 (d, J=7.1 Hz, 1H), 4.56 (d, J=1.4 Hz, 1H), 4.12 (s, 2H), 3.97 (dd, J=11.8, 8.0 Hz, 4H), 3.77 (t, J=7.5 Hz, 2H), 2.42 (dd, J=12.9, 9.2 Hz, 1H), 2.20 (dd, J=13.0, 6.6 Hz, 1H), 1.59 (t, J=17.7 Hz, 3H). LC/MS (ESI) m/z: 502 (M+H)⁺.

### Scheme 284: Synthesis of (S)—N-((4-carbamimidoylthiophen-2-yl)methyl)-6-((4-phenoxybutanoyl) glycyl)-2,6-diazaspiro[3.4]octane-7-carboxamide (Compound 273)

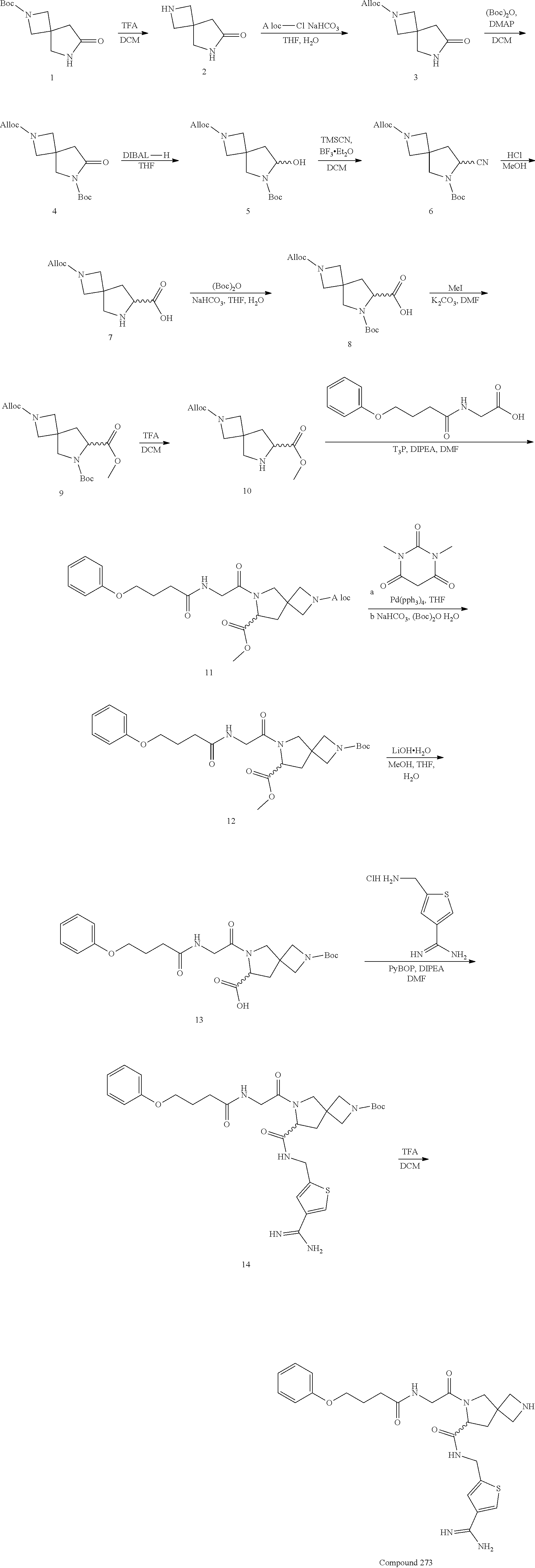

### Step 1: 2,6-diazaspiro[3.4]octan-7-one

A solution of tert-butyl 7-oxo-2,6-diazaspiro[3.4]octane-2-carboxylate (2.5 g, 11.05 mmol) in DCM (25 mL) and TFA (8 mL) was stirred at 25° C. for 2 hours. The mixture was concentrated to dryness under reduced pressure to give 2,6-diazaspiro[3.4]octan-7-one (1.3 g, yield 93.3%) as a white yellow oil, which was directly used in the next reaction without further purification. LC/MS (ESI) m/z: 127 (M+H)⁺.

### Step 2: allyl 7-oxo-2,6-diazaspiro[3.4]octane-2-carboxylate

To a solution of 2,6-diazaspiro[3.4]octan-7-one (1.3 g, 10.30 mmol) in THF (13 mL) and H₂O (4 mL) was added NaHCO₃ (8.7 g, 103.05 mmol) and AllocCl (1.4 g, 11.34 mmol) at 0° C. and the mixture was stirred at 25° C. for 16 hours. The mixture was diluted with EtOAc, washed with water and brine, dried over anhydrous Na₂SO₄, filtered, and concentrated under reduced pressure to give allyl 7-oxo-2,6-diazaspiro[3.4]octane-2-carboxylate (2.1 g, yield 96.9%) as a colorless oil, which was used directly in the next reaction. LC/MS (ESI) (m/z): 211 (M+H)⁺.

### Step 3: 2-allyl 6-(tert-butyl) 7-oxo-2,6-diazaspiro[3.4]octane-2,6-dicarboxylate

To a mixture of allyl 7-oxo-2,6-diazaspiro[3.4]octane-2-carboxylate (2.1 g, 10.0 mmol) in DCM (25 mL) was added (Boc)₂O (6.5 g, 30.0 mmol) followed by DMAP (61 mg, 0.50 mmol) at 0° C., and the mixture was stirred at 25° C. for 3 hours. The mixture was washed with water and brine, dried over anhydrous Na₂SO₄, filtered, and concentrated to dryness. The residue was purified by flash chromatography (silica gel, 0-5% MeOH in DCM) to give 2-allyl 6-(tert-butyl) 7-oxo-2,6-diazaspiro[3.4]octane-2,6-dicarboxylate (1.7 g, yield 54.8%) as a yellow oil. LC/MS (ESI) (m/z): 311 (M+H)⁺.

### Step 4: 2-allyl 6-(tert-butyl) 7-hydroxy-2,6-diazaspiro[3.4]octane-2,6-dicarboxylate

To a solution of 2-allyl 6-(tert-butyl) 7-oxo-2,6-diazaspiro[3.4]octane-2,6-dicarboxylate (1.7 g, 5.48 mmol) in THE (20 mL) was added DIBAL-H (11 mL, 11 mmol, 1 M in hexane) drop-wisely at −70° C. and the mixture was stirred at −70° C. for 1.5 hours. The mixture was quenched with ice-water and extracted with EtOAc twice. The combined organic layers were washed with brine, dried with anhydrous Na₂SO₄, filtered, and concentrated under reduce pressure to give 2-allyl 6-(tert-butyl) 7-hydroxy-2,6-diazaspiro[3.4]octane-2,6-dicarboxylate (1.4 g, yield 81.8%) as a yellow oil, which was used directly in the next reaction without further purification. LC/MS (ESI) (m/z): 335 (M+Na)⁺.

### Step 5: 2-allyl 6-(tert-butyl) 7-cyano-2,6-diazaspiro[3.4]octane-2,6-dicarboxylate

To a mixture of 2-allyl 6-(tert-butyl) 7-hydroxy-2,6-diazaspiro[3.4]octane-2,6-dicarboxylate (1.4 g, 4.48 mmol) in DCM (15 mL) was added TMSCN (1.1 g, 11.21 mmol) followed by BF₃·Et₂O (1.4 g, 9.86 mmol) at −70° C. and the mixture was stirred at −70° C. for 1.5 hours. The mixture was quenched with saturated aq·NaHCO₃ solution and extracted with EtOAc twice. The combined organic layers were washed with brine, dried over anhydrous Na₂SO₄, filtered, and concentrated to dryness. The residue was purified by flash chromatography (silica gel, 0-10% EtOAc in PE) to give 2-allyl 6-(tert-butyl) 7-cyano-2,6-diazaspiro[3.4]octane-2,6-dicarboxylate (840 mg, yield 58.3%) as a colorless oil. LC/MS (ESI) (m/z): 322 (M+H)⁺.

### Step 6: 2-((allyloxy)carbonyl)-2,6-diazaspiro[3.4]octane-7-carboxylic acid

A solution of 2-allyl 6-(tert-butyl) 7-cyano-2,6-diazaspiro[3.4]octane-2,6-dicarboxylate (300 mg, 0.93 mmol) in HCl/MeOH (5 mL, 1 M) was stirred at 25° C. for 2 hours. The mixture was neutralized with 2 N aq·NaOH solution to pH ˜6 and extracted with EtOAc twice. The combined organic layers were concentrated under reduced pressure to dryness to give 2-((allyloxy)carbonyl)-2,6-diazaspiro[3.4]octane-7-carboxylic acid (200 mg, yield 60.5%) as a white solid, which was directly used in the next reaction without further purification. LC/MS (ESI) (m/z): 241 (M+H)⁺.

### Step 7: 2-((allyloxy)carbonyl)-6-(tert-butoxycarbonyl)-2,6-diazaspiro[3.4]octane-7-carboxylic acid

To a solution of 2-((allyloxy)carbonyl)-2,6-diazaspiro[3.4]octane-7-carboxylic acid (200 mg, 0.83 mmol) in THE (6 mL) and H₂O (2 mL) was added NaHCO₃ (699 mg, 8.92 mmol) and (Boc)₂O (550 mg, 2.50 mmol) at 0° C. and the mixture was stirred at 25° C. for 2 hours. The mixture was diluted with H₂O and washed with EtOAc twice. The aqueous layers were acidified with 0.5 M aq·HCl to pH ˜3 and extracted with EtOAc twice. The combined organic layers were washed with brine, dried with anhydrous Na₂SO₄, filtered, and concentrated under reduced pressure to dryness to give 2-((allyloxy)carbonyl)-6-(tert-butoxycarbonyl)-2,6-diazaspiro[3.4]octane-7-carboxylic acid (200 mg, yield 70.6%) as a colorless oil, which was used directly in the next step without purification. LC/MS (ESI) (m/z): 341 (M+H)⁺.

### Step 8: 2-allyl 6-(tert-butyl) 7-methyl 2,6-diazaspiro[3.4]octane-2,6,7-tricarboxylate

To a solution of 2-((allyloxy)carbonyl)-6-(tert-butoxycarbonyl)-2,6-diazaspiro[3.4]octane-7-carboxylic acid (200 mg, 0.59 mmol) in DMF (3 mL) was added Iodomethane (125 mg, 0.88 mmol) and K₂CO₃ (244 mg, 1.76 mmol) at 0° C. under N₂ atmosphere and the mixture was stirred at 25° C. for 1 hour. The mixture was quenched with water and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give 2-allyl 6-(tert-butyl) 7-methyl 2,6-diazaspiro[3.4]octane-2,6,7-tricarboxylate (140 mg, yield 67.2%) as a colorless oil. LC/MS (ESI) m/z: 355 (M+H)⁺.

### Step 9: 2-allyl 7-methyl 2,6-diazaspiro[3.4]octane-2,7-dicarboxylate

A solution of 2-allyl 6-(tert-butyl) 7-methyl 2,6-diazaspiro[3.4]octane-2,6,7-tricarboxylate (70 mg, 0.20 mmol) in DCM (2 mL) and TFA (1 mL) was stirred at room temperature for 2 hours. The mixture was concentrated to dryness under reduced pressure to give 2-allyl 7-methyl 2,6-diazaspiro[3.4]octane-2,7-dicarboxylate (50 mg yield 99.6%) as a colorless oil, which was directly used in the next reaction without further purification. LC/MS (ESI) m/z:255 (M+H)⁺.

### Step 10: 2-allyl 7-methyl-6-((4-phenoxybutanoyl)glycyl)-2,6-diazaspiro[3.4]octane-2,7-dicarboxylate

To a mixture of 2-allyl 7-methyl 2,6-diazaspiro[3.4]octane-2,7-dicarboxylate (50 mg, 0.20 mmol) and (4-phenoxybutanoyl)glycine (42 mg, 0.18 mmol) in DMF (3 mL) was added DIPEA (127 mg, 0.98 mmol) and T₃P (94 mg, 0.30 mmol, 50% wt. in EtOAc) under N₂ atmosphere and the mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (PE:EtOAc=1:1) to give 2-allyl 7-methyl-6-((4-phenoxybutanoyl)glycyl)-2,6-diazaspiro[3.4]octane-2,7-dicarboxylate (50 mg, yield 59.7%) as a colorless oil. LC/MS (ESI) m/z:474 (M+H)⁺.

### Step 11: 2-(tert-butyl) 7-methyl-6-((4-phenoxybutanoyl)glycyl)-2,6-diazaspiro[3.4]octane-2,7-dicarboxylate

To a solution of 2-allyl 7-methyl (S)-6-((4-phenoxybutanoyl)glycyl)-2,6-diazaspiro[3.4]octane-2,7-dicarboxylate (50 mg, 0.11 mmol) in THF (4 mL) was added 1,3-dimethylpyrimidine-2,4,6(1H,3H,5H)-trione (20 mg, 0.13 mmol) and Pd(PPh₃)₄ (6 mg, 0.01 mmol) under N₂ atmosphere and the mixture was stirred at room temperature for 0.5 hour. The mixture was diluted with water (3 mL), NaHCO₃ (89 mg, 1.06 mmol) and (Boc)₂O (69 mg, 0.32 mmol) were added. The resulting mixture was stirred at 25° C. for 2 hours. The mixture was diluted with EtOAc, washed with water and brine, dried over Na₂SO₄, filtered, and concentrated to dryness. The residue was purified by prep-TLC (PE:EtOAc=1:1) to give 2-allyl 7-methyl-6-((4-phenoxybutanoyl)glycyl)-2,6-diazaspiro[3.4]octane-2,7-dicarboxylate (27 mg, yield 52.2%) as a yellow solid. LC/MS (ESI) m/z: 490 (M+H)⁺.

### Step 12: 2-(tert-butoxycarbonyl)-6-((4-phenoxybutanoyl)glycyl)-2,6-diazaspiro[3.4]octane-7-carboxylic acid

To a solution of 2-(tert-butyl) 7-methyl-6-((4-phenoxybutanoyl)glycyl)-2,6-diazaspiro[3.4]octane-2,7-dicarboxylate (27 mg, 0.06 mmol) in MeOH (1 mL) and THF (0.5 mL) was added a solution of LiOH·H₂O (7 mg, 0.17 mmol) in H₂O (0.5 mL) at 0° C. and the mixture was stirred at 25° C. for 4 hours. The mixture was concentrated to dryness under reduced pressure to give 2-(tert-butoxycarbonyl)-6-((4-phenoxybutanoyl) glycyl)-2,6-diazaspiro[3.4]octane-7-carboxylic acid (23 mg, yield 87.7%) as a white solid, which was used directly in the next step. LC/MS (ESI) m/z: 476 (M+H)⁺.

### Step 13: tert-butyl-7-(((4-carbamimidoylthiophen-2-yl)methyl)carbamoyl)-6-((4-phenoxybutanoyl)glycyl)-2,6-diazaspiro[3.4]octane-2-carboxylate

To a mixture of 2-(tert-butoxycarbonyl)-6-((4-phenoxybutanoyl)glycyl)-2,6-diazaspiro [3.4]octane-7-carboxylic acid (20 mg, 0.04 mmol) and 5-(aminomethyl)thiophene-3-carboximidamide hydrochloride (12 mg, 0.06 mmol) in DMF (2 mL) was added DIPEA (27 mg, 0.21 mmol) and PyBOP (33 mg, 0.06 mmol) at 0° C. under N₂ atmosphere and the mixture was stirred at 25° C. for 30 minutes. The mixture was quenched with water and extracted with EtOAc twice. The combined organic layers were washed with brine, dried over anhydrous Na₂SO₄, filtered, and concentrated to dryness under reduced pressure. The residue was purified by pre-TLC (DCM:MeOH=10:1) to give tert-butyl (S)-7-(((4-carbamimidoylthiophen-2-yl)methyl)carbamoyl)-6-((4-phenoxybutanoyl) glycyl)-2,6-diazaspiro[3.4]octane-2-carboxylate (10 mg, yield 38.8%) as a colorless oil. LC/MS (ESI) m/z: 613 (M+H)⁺.

### Step 14: (S)—N-((4-carbamimidoylthiophen-2-yl)methyl)-6-((4-phenoxybutanoyl) glycyl)-2,6-diazaspiro[3.4]octane-7-carboxamide (Compound 273)

A solution of tert-butyl (S)-7-(((4-carbamimidoylthiophen-2-yl)methyl)carbamoyl)-6-((4-phenoxybutanoyl)glycyl)-2,6-diazaspiro[3.4]octane-2-carboxylate (10 mg, 0.02 mmol) in DCM (1 mL) and TFA (0.5 mL) was stirred at 25° C. for 0.5 hour. The mixture was concentrated under reduced pressure to dryness. The residue was purified by prep-HPLC to give Compound 273 (2.1 mg, yield 25.1%) as a white solid. ¹H NMR (400 MHz, CD₃OD) δ 8.53 (s, 2H), 8.24 (s, 1H), 7.46 (d, J=23.3 Hz, 1H), 7.24 (t, J=7.7 Hz, 2H), 6.89 (d, J=6.0 Hz, 3H), 4.58-4.48 (m, 3H), 4.11-3.93 (m, 9H), 3.86 (d, J=10.7 Hz, 1H), 2.45 (dd, J=24.6, 16.9 Hz, 4H), 2.12-2.01 (m, 2H). LC/MS (ESI) m/z: 513 (M+H)⁺.

### Scheme 285: Synthesis of (1S,3S,5S)-5-((1H-1,2,3-triazol-1-yl)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 274)

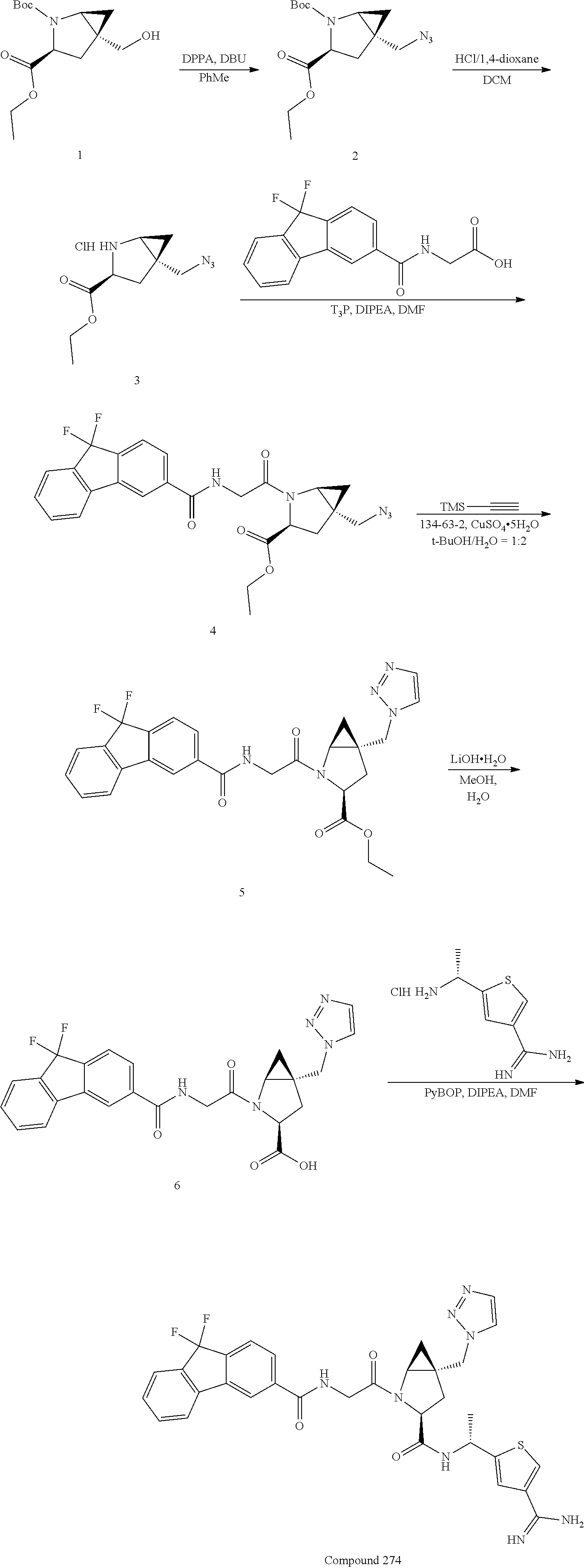

### Step 1: 2-(tert-butyl) 3-ethyl (1S,3S,5S)-5-(azidomethyl)-2-azabicyclo[3.1.0]hexane-2,3-dicarboxylate

To a mixture of 2-(tert-butyl) 3-ethyl (1S,3S,5R)-5-(hydroxymethyl)-2-azabicyclo [3.1.0]hexane-2,3-dicarboxylate (125 mg, 0.44 mmol) and DBU (200 mg, 1.32 mol) in toluene (3 mL) was added DPPA (182 mg, 0.66 mmol) and the mixture was stirred at room temperature for 1 hour. The mixture was stirred at 100° C. overnight. The reaction mixture was concentrated under reduced pressure to dryness. The residue was purified by flash chromatography (silica gel, 0-30% EtOAc in PE) to give 2-(tert-butyl) 3-ethyl (1 S,3S,5S)-5-(azidomethyl)-2-azabicyclo[3.1.0]hexane-2,3-dicarboxylate (75 mg, yield 54.3%) as a yellow oil. LCMS (ESI) m/z: 311 (M+H)⁺.

### Step 2: ethyl (1S,3S,5S)-5-(azidomethyl)-2-azabicyclo[3.1.0]hexane-3-carboxylate hydrochloride

To a solution of 2-(tert-butyl) 3-ethyl (1 S,3S,5S)-5-(azidomethyl)-2-azabicyclo [3.1.0]hexane-2,3-dicarboxylate (75 mg, 0.24 mmol) in DCM (1 mL) was added HCl/1,4-dioxane (1 mL, 4M) and the mixture was stirred at room temperature for 2 hours. The mixture was concentrated to dryness under reduced pressure to give ethyl (1 S,3S,5S)-5-(azidomethyl)-2-azabicyclo[3.1.0]hexane-3-carboxylate hydrochloride (65 mg, crude) as a white solid, which was directly used in the next reaction without further purification. LC/MS (ESI) m/z: 211 (M+H)⁺.

### Step 3: ethyl (1S,3S,5S)-5-(azidomethyl)-2-((9,9-difluoro-9H-fluorene-3-carbonyl) glycyl)-2-azabicyclo [3.1.0]hexane-3-carboxylate

To a mixture of ethyl (1S,3S,5S)-5-(azidomethyl)-2-azabicyclo[3.1.0]hexane-3-carboxylate hydrochloride (76 mg, 0.31 mmol) and (9,9-difluoro-9H-fluorene-3-carbonyl)glycine (79 mg, 0.26 mmol) in DMF (2 mL) was added DIPEA (168 mg, 1.3 mmol) and T₃P (248 mg, 0.39 mmol, 50% wt. in EtOAc) under N₂ atmosphere and the mixture was stirred at room temperature for 1.5 hours. The reaction mixture was diluted with EtOAc, washed with water and brine, dried over anhydrous Na₂SO₄, filtered, and concentrated under reduced pressure to dryness. The residue was purified by flash chromatography (silica gel, 0-60% EtOAc in PE) to give ethyl (1S,3S,5S)-5-(azidomethyl)-2-((9,9-difluoro-9H-fluorene-3-carbonyl)glycyl)-2-azabicyclo[3.1.0]hexane-3-carboxylate (124 mg, yield 96.1%) as a yellow oil. LCMS (ESI) m/z: 496 (M+H)⁺.

### Step 4: ethyl (1S,3S,5S)-5-((1H-1,2,3-triazol-1-yl)methyl)-2-((9,9-difluoro-9H-fluorene-3-carbonyl)glycyl)-2-azabicyclo[3.1.0]hexane-3-carboxylate

To a mixture of ethyl (1S,3S,5S)-5-(azidomethyl)-2-((9,9-difluoro-9H-fluorene-3-carbonyl)glycyl)-2-azabicyclo[3.1.0]hexane-3-carboxylate (60 mg, 0.12 mmol), ethynyltrimethylsilane (24 mg, 0.24 mmol), sodium ascorbate (134 mg, 0.68 mmol) in t-BuOH (1 mL) and H₂O (1 mL) was added CuSO₄.5H₂O (65 mg, 0.24 mmol) and the reaction mixture was stirred under N₂ atmosphere at 60° C. for 6 hours. The reaction mixture was diluted with EtOAc, washed with water and brine, dried over anhydrous Na₂SO₄, filtered, and concentrated under reduced pressure to dryness. The residue was purified by flash chromatography (silica gel, 0-60% EtOAc in PE) to give ethyl (1S,3S,5S)-5-((1H-1,2,3-triazol-1-yl)methyl)-2-((9,9-difluoro-9H-fluorene-3-carbonyl) glycyl)-2-azabicyclo[3.1.0]hexane-3-carboxylate (30 mg, yield 47.9%) as a yellow oil. LCMS (ESI) m/z: 522 (M+H)⁺.

### Step 5: (1S,3S,5S)-5-((1H-1,2,3-triazol-1-yl)methyl)-2-((9,9-difluoro-9H-fluorene-3-carbonyl)glycyl)-2-azabicyclo[3.1.0]hexane-3-carboxylic acid

To a solution of ethyl (1S,3S,5S)-5-((1H-1,2,3-triazol-1-yl)methyl)-2-((9,9-difluoro-9H-fluorene-3-carbonyl)glycyl)-2-azabicyclo[3.1.0]hexane-3-carboxylate (30 mg, 0.058 mmol) in MeOH (1 mL) and water (0.5 mL) was added LiOH·H₂O (5 mg, 0.12 mmol) at 0° C. and the mixture was stirred at room temperature for 1 hour. The mixture was acidified with 1 N aq·HCl solution to pH˜3 and extracted with EtOAc twice. The combined organic layers were washed with brine, dried over anhydrous Na₂SO₄, filtered, and concentrated under reduced pressure to dryness to give (1S,3S,5S)-5-((1H-1,2,3-triazol-1-yl)methyl)-2-((9,9-difluoro-9H-fluorene-3-carbonyl)glycyl)-2-azabicyclo [3.1.0]hexane-3-carboxylic acid (28 mg, crude) as a white solid, which was used directly in next reaction without further purification. LC/MS (ESI) (m/z): 494(M+H)⁺.

### Step 6: (1S,3S,5S)-5-((1H-1,2,3-triazol-1-yl)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 274)

To a mixture of (1S,3S,5S)-5-((1H-1,2,3-triazol-1-yl)methyl)-2-((9,9-difluoro-9H-fluorene-3-carbonyl)glycyl)-2-azabicyclo[3.1.0]hexane-3-carboxylic acid (28 mg, 0.057 mmol) and (R)-5-(1-aminoethyl)thiophene-3-carboximidamide hydrochloride (24 mg, 0.11 mmol) in DMF (2 mL) was added DIPEA (33 mg, 0.063 mmol) and PyBOP (37 mg, 0.29 mmol) at 0° C. under N₂ atmosphere and the mixture was stirred at room temperature for 2 hours. The reaction mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 274(2.6 mg, yield 4.5%) as a white solid. ¹H NMR (400 MHz, CD₃OD) δ 8.54 (s, 1H), 8.18 (d, J=9.3 Hz, 2H), 8.07 (d, J=20.9 Hz, 1H), 7.88 (d, J=7.7 Hz, 1H), 7.76 (dd, J=16.2, 7.4 Hz, 3H), 7.66 (d, J=7.3 Hz, 1H), 7.58 (t, J=7.6 Hz, 1H), 7.48-7.42 (m, 2H), 5.20 (dd, J=13.7, 6.7 Hz, 1H), 4.81 (d, J=8.1 Hz, 1H), 4.68 (d, J=14.4 Hz, 1H), 4.56 (dd, J=23.1, 15.6 Hz, 2H), 4.33 (d, J=16.6 Hz, 1H), 3.91 (dd, J=6.1, 2.6 Hz, 1H), 2.58 (t, J=12.4 Hz, 1H), 2.09 (dd, J=13.4, 3.4 Hz, 1H), 1.57 (t, J=13.1 Hz, 3H), 1.50 (dd, J=6.1, 2.6 Hz, 1H), 1.23 (t, J=5.6 Hz, 1H). LCMS (ESI) (m/z): 645 (M+H)⁺.

### Scheme 286: Synthesis of (S)—N—((S*)-1-(4-carbamimidoylthiophen-2-yl)-2,2,2-trifluoroethyl)-7-((9,9-difluoro-9H-fluorene-3-carbonyl)glycyl)-1,4-dioxa-7-azaspiro[4.4]nonane-8-carboxamide (Compound 275)

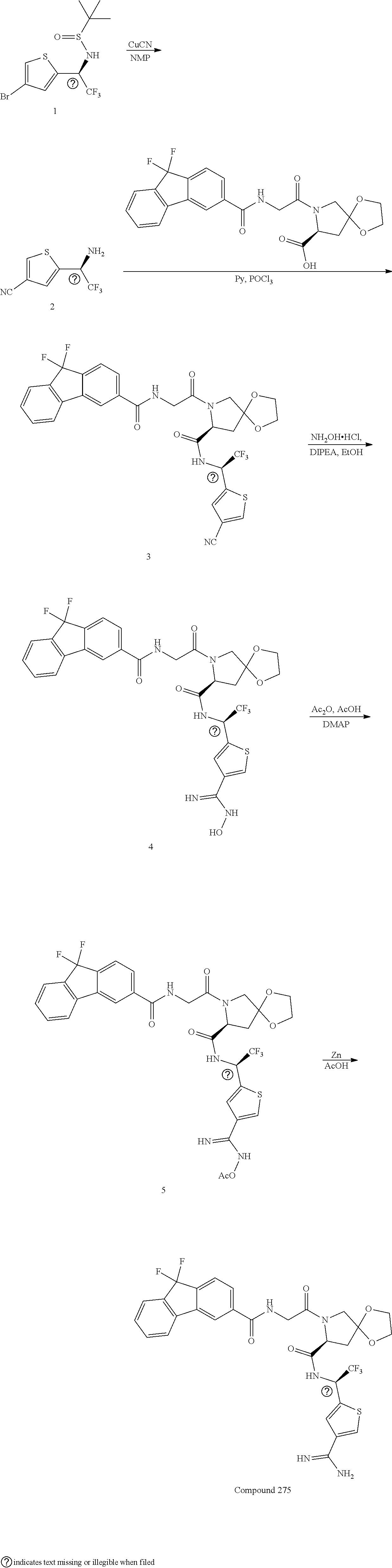

Compound 275 was prepared from (R)—N—((S*)-1-(4-bromothiophen-2-yl)-2,2,2-trifluoroethyl)-2-methylpropane-2-sulfinamide, (S)-7-((9,9-difluoro-9H-fluorene-3-carbonyl)glycyl)-1,4-dioxa-7-azaspiro[4.4]nonane-8-carboxylic acid, and hydroxylamine hydrochloride based on the procedures set forth in Scheme 236. ¹H NMR (400 MHz, CD₃OD) δ 8.35 (d, J=58.7 Hz, 1H), 8.15 (d, J=13.2 Hz, 1H), 7.86-7.82 (m, 1H), 7.72 (dd, J=13.2, 7.7 Hz, 2H), 7.67-7.62 (m, 2H), 7.58 (t, J=7.4 Hz, 1H), 7.45 (t, J=7.2 Hz, 1H), 6.14 (dd, J=15.4, 8.0 Hz, 1H), 4.69 (dd, J=9.1, 5.4 Hz, 1H), 4.27 (d, J=16.4 Hz, 1H), 4.12 (d, J=16.5 Hz, 1H), 4.03-3.95 (m, 4H), 3.85 (q, J=10.8 Hz, 2H), 2.48 (dd, J=13.2, 9.2 Hz, 1H), 2.19 (dd, J=13.4, 5.5 Hz, 1H). LCMS (ESI) m/z: 664 (M+H)⁺.

### Scheme 286: Synthesis of (1S,3S,5S)—N—((R)-1-(4-(2-amino-2-iminoethyl)thiophen-2-yl)ethyl)-5-methyl-2-((4-phenoxybenzoyl)glycyl)-2-azabicyclo[3.1.0]hexane-3-carboxamide (Compound 276)

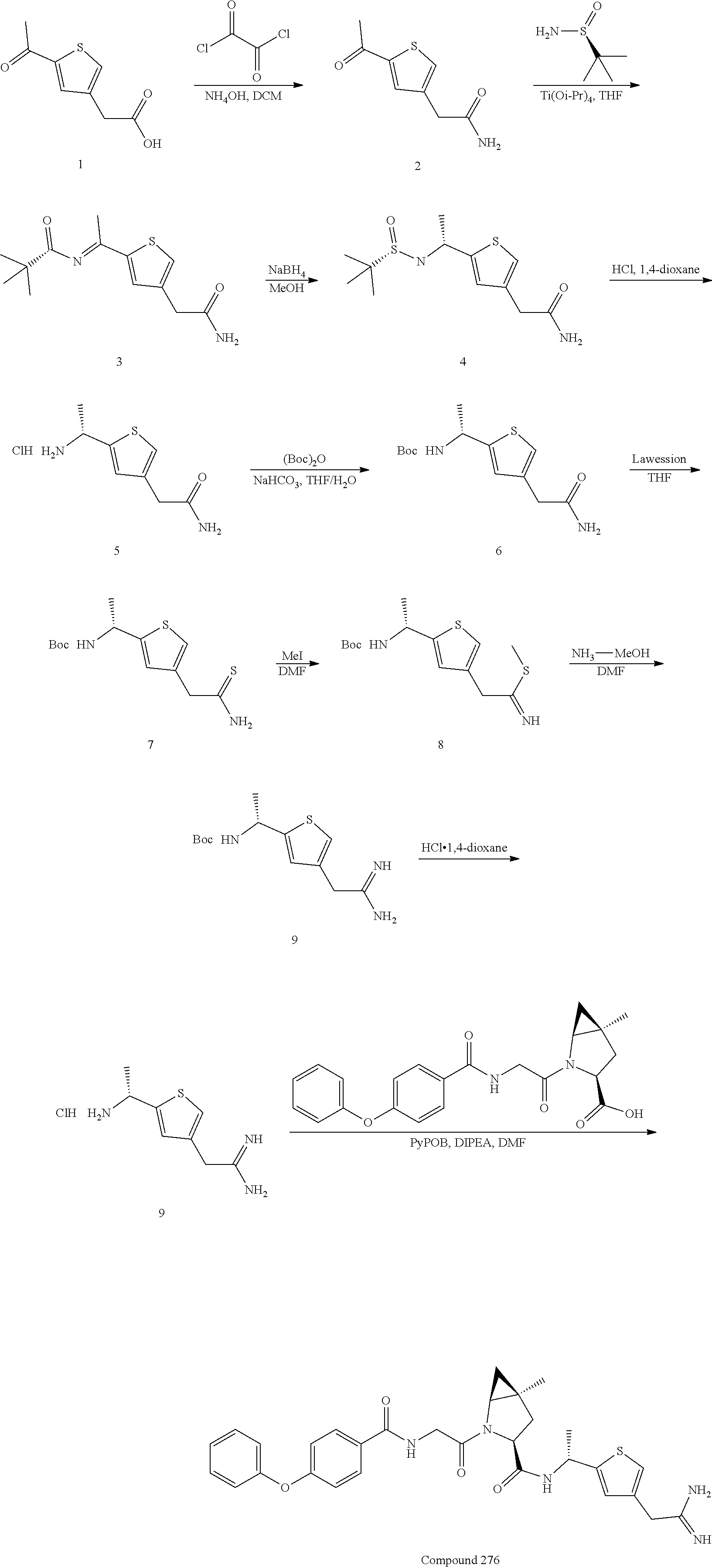

### Step 1: 2-(5-acetylthiophen-3-yl)acetamide

To a solution of 2-(5-acetylthiophen-3-yl)acetic acid (2.0 g, 10.86 mmol) in DCM (24 mL) was added oxalyl chloride (2.07 g, 16.29 mmol) under N₂ atmosphere and the mixture was stirred at room temperature overnight. Then NH₃·H₂O (7.61 g, 217.14 mmol) was added to the solution drop-wise and the reaction stirred at room temperature for 2 hours. The mixture was washed with saturated aq. NaHCO₃ solution and extracted with CHCl₃/i-PrOH (3/1) three times. The combined organic layers were washed with brine, dried over anhydrous Na₂SO₄, filtered, and concentrated to dryness under reduced pressure to give 2-(5-acetylthiophen-3-yl)acetamide (1.8 g, yield 90.5%) as a yellow solid, which was directly used in the next without further purification. LC/MS (ESI) (m/z): 184 (M+H)⁺.

### Step 2: (E)-2-(5-(1-((tert-butylsulfinyl)imino)ethyl)thiophen-3-yl)acetamide

To a solution of 2-(5-acetylthiophen-3-yl)acetamide (1.6 g, 8.73 mmol) in THE (25 mL) was added titanium isopropoxide (14.89 g, 52.39 mmol) followed by (S)-2-methylpropane-2-sulfinamide (3.18 g, 26.20 mmol) and the mixture was stirred at 70° C. for 24 hours. The mixture was washed with water and filtered, the aqueous was extracted with EtOAc twice. The combined organic layers were washed with brine, dried over anhydrous Na₂SO₄, filtered, and concentrated to dryness. The residue was purified by flash chromatography (0-14% MeOH in DCM) to give (E)-2-(5-(1-((tert-butylsulfinyl)imino)ethyl)thiophen-3-yl)acetamide (1.2 g, yield 48.0%) as a yellow solid. LC/MS (ESI) (m/z): 287 (M+H)⁺.

### Step 3: 2-(5-((1R)-1-((tert-butylsulfinyl)amino)ethyl)thiophen-3-yl)acetamide and 2-(5-((1S)-1-((tert-butylsulfinyl)amino)ethyl)thiophen-3-yl)acetamide

To a solution of (E)-2-(5-(1-((tert-butylsulfinyl)imino)ethyl)thiophen-3-yl)acetamide (1.2 g, 4.19 mmol) in MeOH (15 mL) was added NaBH₄ (476 mg, 12.57 mmol) at 0° C., and the mixture was stirred at room temperature for 5 hours. The mixture was diluted with water and extracted with DCM twice. The combined organic layers were washed with brine, dried with anhydrous Na₂SO₄, filtered, and concentrated to dryness. The residue was purified by prep-HPLC to give 2-(5-((1R)-1-((tert-butylsulfinyl)amino) ethyl)thiophen-3-yl)acetamide (700 mg, yield 58.0%) as a light oil and 2-(5-((1 S)-1-((tert-butylsulfinyl)amino)ethyl)thiophen-3-yl)acetamide (350 mg, yield 29.0%) as a yellow oil. LC/MS (ESI) m/z: 289 (M+H)⁺.

### Step 4: (R)-2-(5-(1-aminoethyl)thiophen-3-yl)acetamide hydrochloride

To a solution of 2-(5-((1R)-1-((tert-butylsulfinyl)amino)ethyl)thiophen-3-yl)acetamide (700 mg, 2.43 mmol) in DCM (2 mL) was added HCl/1,4-dioxane (5 mL, 4M) was stirred at room temperature for 1 hour. The mixture was concentrated to dryness under reduced pressure to give (R)-2-(5-(1-aminoethyl)thiophen-3-yl)acetamide hydrochloride (530 mg, yield 98.9%) as a yellow solid, which was directly used in the next step without further purification. LC/MS (ESI) m/z: 185 (M+H)⁺.

### Step 5: tert-butyl (R)-(1-(4-(2-amino-2-oxoethyl)thiophen-2-yl)ethyl)carbamate

To a solution of (R)-2-(5-(1-aminoethyl)thiophen-3-yl)acetamide hydrochloride (530 mg, 2.41 mmol) and NaHCO₃ (2.02 g, 24.1 mmol) in THE (9 mL)/H₂O (3 mL) was added (Boc)₂O (1.58 g, 7.23 mmol) at 0° C. and the mixture was stirred at room temperature overnight. The mixture was diluted with water and extracted with EtOAc twice. The combined organic layers were washed with brine, dried with anhydrous Na₂SO₄, filtered, and concentrated to dryness, The residue was purified by flash chromatography (0-8% MeOH in DCM) to give tert-butyl (R)-(1-(4-(2-amino-2-oxoethyl)thiophen-2-yl)ethyl)carbamate (400 mg, yield 58.5%) as a white solid. LC/MS (ESI) m/z: 285 (M+H)⁺.

### Step 6: tert-butyl (R)-(1-(4-(2-amino-2-thioxoethyl)thiophen-2-yl)ethyl)carbamate

To a solution of tert-butyl (R)-(1-(4-(2-amino-2-oxoethyl)thiophen-2-yl)ethyl) carbamate (250 mg, 0.88 mmol) in THF(6 mL) was added Lawesson's reagent (711 mg, 1.76 mmol) and the mixture was stirred at room temperature for 3 hours. The mixture was concentrated to dryness. The residue was purified by flash chromatography (0-10% MeOH in DCM) to give tert-butyl (R)-(1-(4-(2-amino-2-thioxoethyl)thiophen-2-yl)ethyl)carbamate (168 mg, yield 63.6%) as a light oil. LC/MS (ESI) m/z: 301 (M+H)⁺.

### Step 7: methyl (R)-2-(5-(1-((tert-butoxycarbonyl)amino)ethyl)thiophen-3-yl) ethanimidothioate

To a solution of tert-butyl (R)-(1-(4-(2-amino-2-thioxoethyl)thiophen-2-yl)ethyl) carbamate (160 mg, 0.53 mmol) in DMF (3 mL) was added MeI (0.35 mL) and the mixture was stirred at 0° C. for 1 hour. The mixture was concentrated to dryness under reduced pressure to give methyl (R)-2-(5-(1-((tert-butoxycarbonyl)amino)ethyl) thiophen-3-yl)ethanimidothioate (165 mg, yield 98.5%) as a light oil, which was directly used in the next reaction without further purification. LC/MS (ESI) m/z: 315 (M+H)⁺.

### Step 8: tert-butyl (R)-(1-(4-(2-amino-2-iminoethyl)thiophen-2-yl)ethyl)carbamate

To a solution of methyl (R)-2-(5-(1-((tert-butoxycarbonyl)amino)ethyl)thiophen-3-yl)ethanimidothioate (165 mg, 0.53 mmol) in DMF (3 mL) was added NH₃·MeOH (0.8 mL, 7 M), the mixture was stirred at 0° C. for 1 hour. The mixture was concentrated to dryness. The residue was purified by prep-TLC (DCM:MeOH=10:1) to give tert-butyl (R)-(1-(4-(2-amino-2-iminoethyl)thiophen-2-yl)ethyl)carbamate (60 mg, yield 40.4%) as a white solid. LC/MS (ESI) m/z: 284 (M+H)⁺.

### Step 9: (R)-2-(5-(1-aminoethyl)thiophen-3-yl)acetimidamide hydrochloride

A solution of tert-butyl (R)-(1-(4-(2-amino-2-iminoethyl)thiophen-2-yl)ethyl) carbamate (50 mg, 0.18 mmol) in HCl/1,4-dioxane (3 mL, 4 M) was stirred under N₂ atmosphere at room temperature for 1 hour. The reaction mixture was concentrated to dryness under reduced pressure, co-evaporated with DCM and dried under vacuum to give (R)-2-(5-(1-aminoethyl)thiophen-3-yl)acetimidamide hydrochloride (38 mg, yield 98.2%) as a yellow solid, which was used directly in the next reaction without further purification. LC/MS (ESI) m/z: 184 (M+H)⁺.

### Step 10: (1S,3S,5S)—N—((R)-1-(4-(2-amino-2-iminoethyl)thiophen-2-yl)ethyl)-5-methyl-2-((4-phenoxybenzoyl)glycyl)-2-azabicyclo[3.1.0]hexane-3-carboxamide (Compound 276)

To a mixture of(1 S,3S,5S)-5-methyl-2-((4-phenoxybenzoyl)glycyl)-2-azabicyclo[3.1.0]hexane-3-carboxylic acid (67 mg, 0.17 mmol) and (R)-2-(5-(1-aminoethyl)thiophen-3-yl)acetimidamide hydrochloride (38 mg, 0.17 mmol) in DMF (3 mL) was added DIPEA (88 mg, 0.68 mmol) and PyBOP (97 mg, 0.19 mmol) at 0° C. and the mixture was stirred under N₂ atmosphere at room temperature for 1 hour. The mixture was diluted with EtOAc, washed with water and brine, dried over anhydrous Na₂SO₄, filtered, and concentrated to dryness under reduced pressure. The residue was purified by prep-TLC (DCM:MeOH=10:1) and further purified by prep-HPLC to give (1S,3S,5S)—N—((R)-1-(4-(2-amino-2-iminoethyl)thiophen-2-yl)ethyl)-5-methyl-2-((4-phenoxybenzoyl) glycyl)-2-azabicyclo[3.1.0]hexane-3-carboxamide (8.0 mg, yield 8.7%) as a white solid. ¹H NMR (400 MHz, CD₃OD) δ 7.89-7.83 (m, 2H), 7.45-7.38 (m, 2H), 7.24-7.17 (m, 2H), 7.09-7.04 (m, 2H), 7.03-6.99 (m, 3H), 5.28-5.14 (m, 1H), 4.83-4.80 (m, 1H), 4.44-4.23 (m, 2H), 3.76-3.62 (m, 2H), 3.42-3.34 (m, 1H), 2.65-2.36 (m, 1H), 2.30-2.11 (m, 1H), 1.54 (dd, J=14.8 Hz, 14.8 Hz, 3H), 1.28 (d, J=11.6 Hz, 3H), 1.22 (dd, J=5.6, 5.6 Hz, 1H), 0.76 (t, J=5.2 Hz, 1H). LC/MS (ESI) m/z: 560 (M+H)⁺.

### Scheme 287: Synthesis of (8S)—N—((R)-1-(4-carbamimidoylthiophen-2-yl)ethyl)-7-((9-methoxy-9H-fluorene-3-carbonyl)glycyl)-1,4-dioxa-7-azaspiro[4.4]nonane-8-carboxamide (Compound 277)

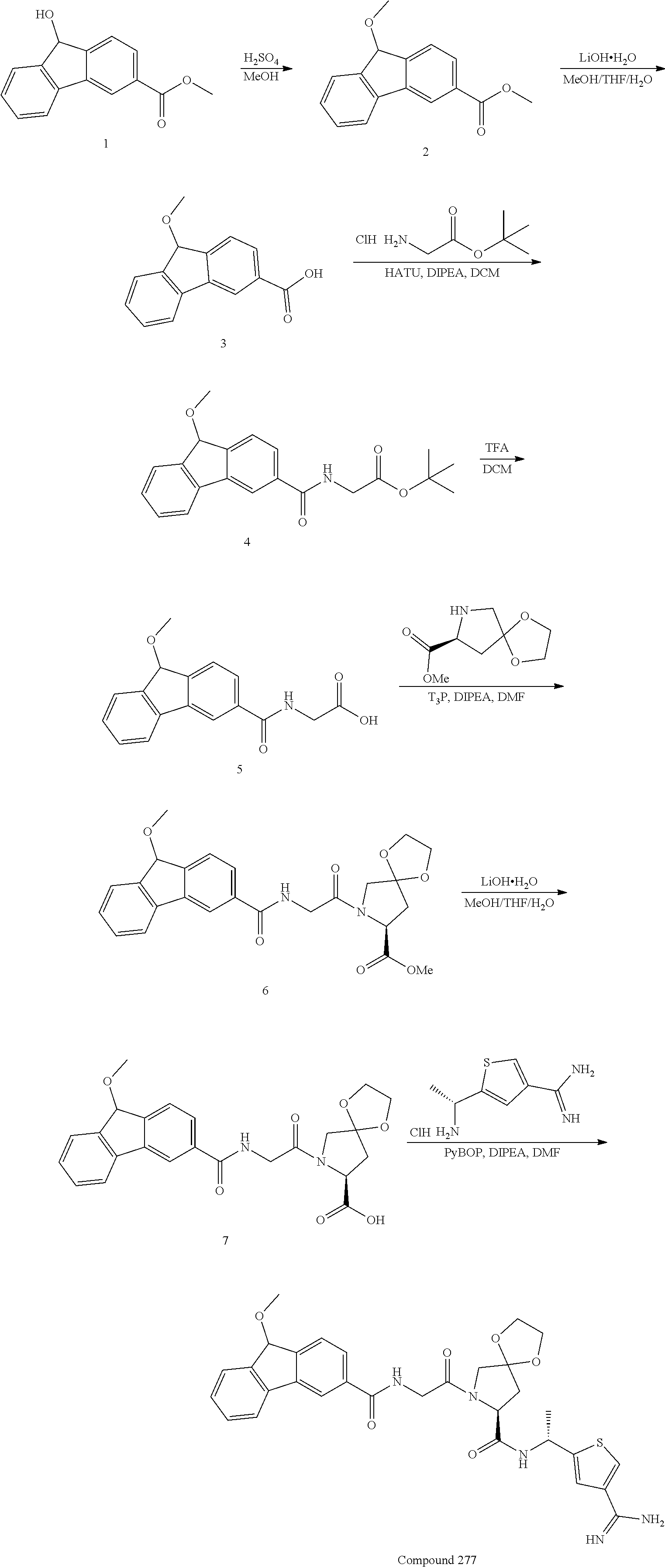

### Step 1: methyl 9-methoxy-9H-fluorene-3-carboxylate

To a solution of methyl 9-hydroxy-9H-fluorene-3-carboxylate (770 mg, 3.21 mmol) in MeOH (9 mL) was added H₂SO₄ (1 mL) at 0° C. and the mixture was stirred at 80° C. for 16 hours. The mixture was quenched with saturated aq·NaHCO₃ solution and extracted with EtOAc twice. The combined organic layers were washed with brine, dried over anhydrous Na₂SO₄, filtered, and concentrated under reduced pressure. The residue was purified by flash chromatography (silica gel, 0-25% EtOAc in PE) to give methyl 9-methoxy-9H-fluorene-3-carboxylate (650 mg, yield 79.8%) as a white solid. ¹H NMR (400 MHz, CDCl3) δ 8.33 (s, 1H), 8.03 (dd, J=7.8, 1.2 Hz, 1H), 7.75 (d, J=7.5 Hz, 1H), 7.65 (dd, J=20.0, 20.0 Hz, 2H), 7.44 (t, J=7.4 Hz, 1H), 7.37 (t, J=7.4 Hz, 1H), 5.64 (s, 1H), 3.97 (s, 3H), 3.07 (s, 3H). LC/MS (ESI) m/z: 255 (M+H)⁺.

### Step 2: 9-methoxy-9H-fluorene-3-carboxylic acid

To a solution of methyl 9-methoxy-9H-fluorene-3-carboxylate (650 mg, 2.56 mmol) in MeOH (4 mL) and THE (2 mL) was added a solution of LiOH·H₂O (430 mg, 10.23 mmol) in water (2 mL) at 0° C. and the mixture was stirred at room temperature for 2 hours. The mixture was diluted with water and extracted with EtOAc twice. The aqueous layer was acidified with 1 N aq·HCl to pH ˜4 and extracted with EtOAc twice. The combined organic layers were washed with brine, dried over anhydrous Na₂SO₄, filtered, and concentrated to dryness under reduced pressure to give 9-methoxy-9H-fluorene-3-carboxylic acid (300 mg, yield 48.8%) as a white solid, which was used directly in the next step. LC/MS (ESI) m/z: 241 (M+H)⁺.

### Step 3: tert-butyl (9-methoxy-9H-fluorene-3-carbonyl)glycinate

To a mixture of 9-methoxy-9H-fluorene-3-carboxylic acid (200 mg, 0.83 mmol) and tert-butyl glycinate hydrochloride (167 mg, 1.0 mmol) in DCM (5 mL) was added HATU (475 mg, 1.25 mmol) and DIPEA (430 mg, 3.32 mmol) under N₂ atmosphere and the reaction was stirred at room temperature for 2 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-80% EtOAc in PE) to give tert-butyl (9-methoxy-9H-fluorene-3-carbonyl)glycinate (300 mg, yield 93.0%) as a light oil. LC/MS (ESI) m/z: 354 (M+H)⁺.

### Step 4: (9-methoxy-9H-fluorene-3-carbonyl)glycine

To a solution of tert-butyl (9-methoxy-9H-fluorene-3-carbonyl)glycinate (100 mg, 0.28 mmol) in DCM (2 mL) was added TFA (1 mL) and the solution was stirred under N₂ atmosphere at room temperature for 2 hours. The reaction mixture was concentrated to dryness under reduced pressure to give (9-methoxy-9H-fluorene-3-carbonyl)glycine (80 mg, yield 95.1%) as a yellow oil, which was used directly in the next step without further purification. LC/MS (ESI) m/z: 298 (M+H)⁺.

### Step 5: methyl (8S)-7-((9-methoxy-9H-fluorene-3-carbonyl)glycyl)-1,4-dioxa-7-azaspiro[4.4]nonane-8-carboxylate

To a mixture of (9-methoxy-9H-fluorene-3-carbonyl)glycine (80 mg, 0.27 mmol) and methyl (S)-1,4-dioxa-7-azaspiro[4.4]nonane-8-carboxylate (100 mg, 0.54 mmol) in DMF (3 mL) was added DIPEA (139 mg, 1.08 mmol) and T₃P (257 mg, 0.40 mmol, 50% wt. in EtOAc) under N₂ atmosphere and the mixture was stirred at room temperature for 3 hours. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-80% EtOAc in PE) to give methyl (8S)-7-((9-methoxy-9H-fluorene-3-carbonyl)glycyl)-1,4-dioxa-7-azaspiro[4.4]nonane-8-carboxylate (90 mg, yield 71.7%) as a light oil. LC/MS (ESI) m/z: 467 (M+H)⁺.

### Step 6: (8S)-7-((9-methoxy-9H-fluorene-3-carbonyl)glycyl)-1,4-dioxa-7-azaspiro[4.4]nonane-8-carboxylic acid

To a solution of methyl (8S)-7-((9-methoxy-9H-fluorene-3-carbonyl)glycyl)-1,4-dioxa-7-azaspiro[4.4]nonane-8-carboxylate (90 mg, 0.19 mmol) in MeOH (2 mL) and THE (1 mL) was added a solution of LiOH·H₂O (32 mg, 0.77 mmol) in water (1 mL) at 0° C. and the mixture was stirred at room temperature for 0.5 hour. The mixture was diluted with water and extracted with EtOAc twice. The aqueous layer was acidified with 1 N aq·HCl to pH¬4 and extracted with EtOAc twice. The combined organic layers were washed with brine, dried over anhydrous Na₂SO₄, filtered, and concentrated to dryness under reduced pressure. The residue was purified by prep-HPLC to give (8S)-7-((9-methoxy-9H-fluorene-3-carbonyl)glycyl)-1,4-dioxa-7-azaspiro[4.4]nonane-8-carboxylic acid (60 mg, yield 68.7%) as a light oil. LC/MS (ESI) m/z: 453 (M+H)⁺.

### Step 7: (8S)—N—((R)-1-(4-carbamimidoylthiophen-2-yl)ethyl)-7-((9-methoxy-9H-fluorene-3-carbonyl)glycyl)-1,4-dioxa-7-azaspiro[4.4]nonane-8-carboxamide (Compound 277)

To a mixture of (8S)-7-((9-methoxy-9H-fluorene-3-carbonyl)glycyl)-1,4-dioxa-7-azaspiro[4.4]nonane-8-carboxylic acid (30 mg, 0.066 mmol) and (R)-5-(1-aminoethyl)thiophene-3-carboximidamide hydrochloride (27 mg, 0.13 mmol) in DMF (2 mL) was added DIPEA (34 mg, 0.27 mmol) and PyBOP (38 mg, 0.073 mmol) at 0° C. and the mixture was stirred under N₂ atmosphere at room temperature for 1 hour. The mixture was diluted with EtOAc, washed with water and brine, dried over anhydrous Na₂SO₄, filtered, and concentrated to dryness under reduced pressure. The residue was purified by prep-TLC (DCM:MeOH=10:1) and further purified by prep-HPLC to give (8S)—N—((R)-1-(4-carbamimidoylthiophen-2-yl)ethyl)-7-((9-methoxy-9H-fluorene-3-carbonyl)glycyl)-1,4-dioxa-7-azaspiro[4.4]nonane-8-carboxamide (6.0 mg, yield 15.0%) as a white solid. ¹H NMR (400 MHz, CD₃OD) δ 8.26-8.18 (m, 2H), 7.83 (d, J=8.0 Hz, 1H), 7.77 (d, J=7.2 Hz, 1H), 7.68 (d, J=8.0 Hz, 1H), 7.63 (d, J=7.6 Hz, 1H), 7.53 (d, J=10.4 Hz, 1H), 7.45 (t, J=7.4 Hz, 1H), 7.38 (t, J=7.4 Hz, 1H), 5.60 (s, 1H), 5.28 (q, J=6.8 Hz, 1H), 4.60-4.55 (m, 1H), 4.25-4.15 (m, 2H), 4.02-3.95 (m, 4H), 3.84-3.77 (m, 2H), 3.16 (d, J=4.8 Hz, 3H), 2.49-2.39 (m, 1H), 2.25-2.15 (m, 1H), 1.63 (dd, J=38.0, 38.0 Hz, 3H). LC/MS (ESI) m/z: 453 (M+H)⁺.

### Scheme 288: Synthesis of (2S,4R)-1-((anthracene-2-carbonyl)glycyl)-N—((R)-1-(4-carbamimidoylthiophen-2-yl)ethyl)-4-methoxypyrrolidine-2-carboxamide (Compound 278)

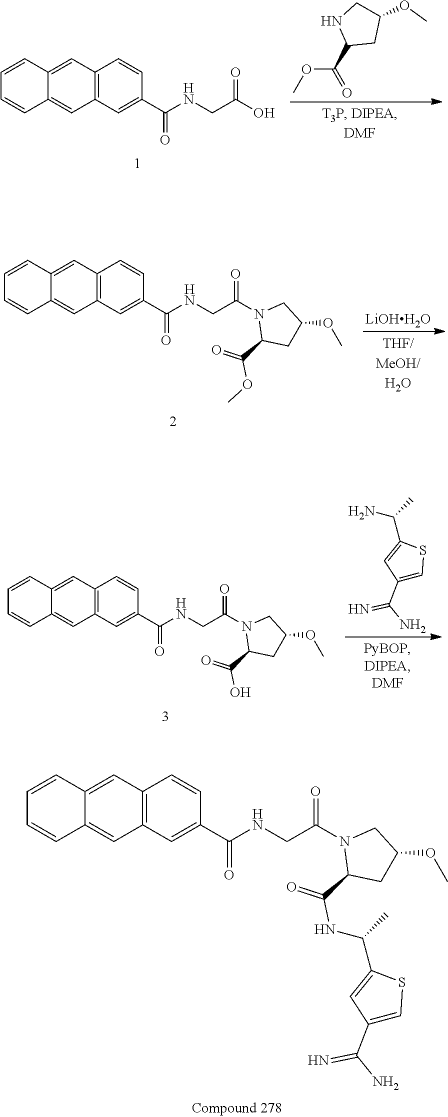

Compound 278 was prepared from (anthracene-2-carbonyl)glycine, methyl (2S,4R)-4-methoxypyrrolidine-2-carboxylate, and (R)-5-(1-aminoethyl)thiophene-3-carboximidamide based on the procedures set forth in Scheme 3. ¹H NMR (400 MHz, CD₃OD) δ 8.61 (d, J=7.6 Hz, 3H), 8.53 (s, 1H), 8.22 (dd, J=20.8, 1.2 Hz, 1H), 8.13-8.05 (m, 3H), 7.85 (dt, J=9.8, 4.9 Hz, 1H), 7.58-7.50 (m, 3H), 5.31 (dq, J=13.7, 6.7 Hz, 1H), 4.52 (t, J=8.1 Hz, 1H), 4.38 (d, J=16.8 Hz, 1H), 4.21 (dd, J=22.8, 9.8 Hz, 2H), 3.96-3.80 (m, 2H), 3.40 (s, 3H), 2.46 (dd, J=36.4, 25.1 Hz, 1H), 2.23-2.04 (m, 1H), 1.61 (t, J=17.3 Hz, 3H). LC/MS (ESI) m/z:558 (M+H)⁺.

### Scheme 289: Synthesis of (S)—N—((R)-1-(4-carbamimidoylthiophen-2-yl)ethyl)-7-((dibenzo[b,d]furan-3-carbonyl)glycyl)-1,4-dioxa-7-azaspiro[4.4]nonane-8-carboxamide (Compound 279)

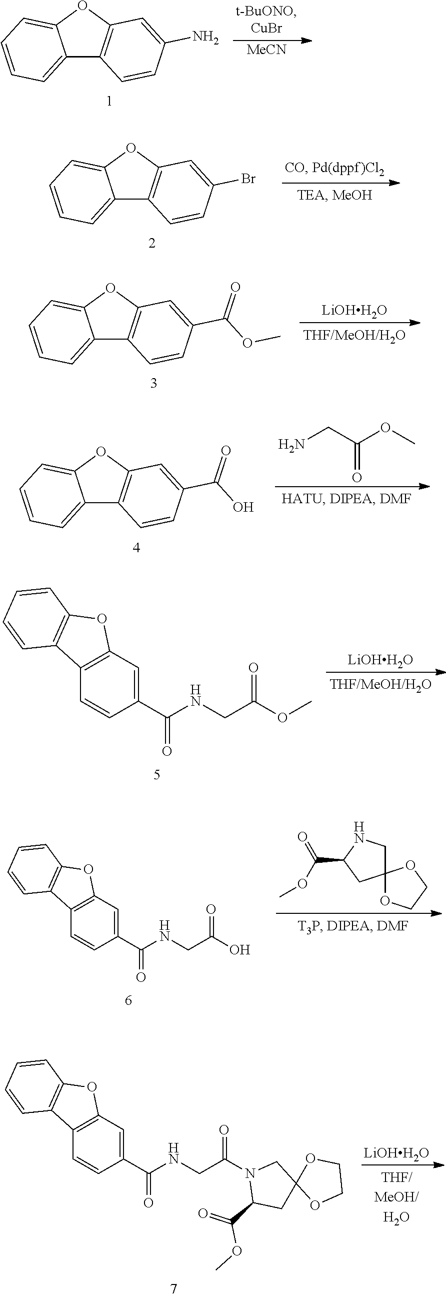

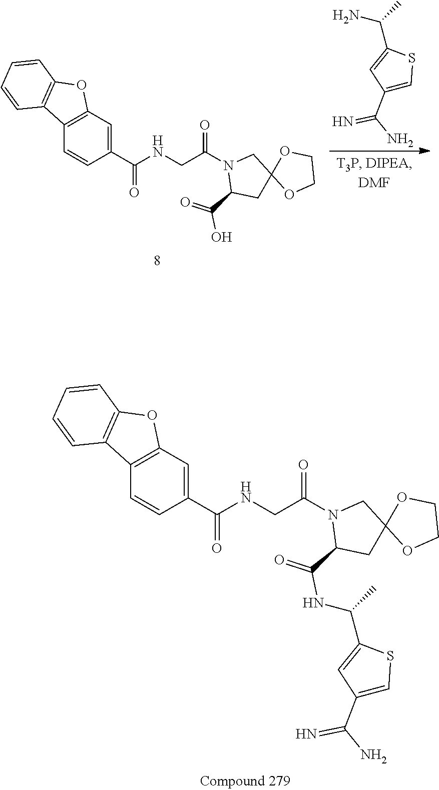 [SMILES: O=C(NCC(=O)N1CC2(C[C@H]1C(=O)O)OCCO2)c1ccc2c(c1)oc1ccccc12 ; C[C@@H](N)c1cc(C(=N)N)cs1 ; C[C@@H](NC(=O)[C@@H]1CC2(CN1C(=O)CNC(=O)c1ccc3c(c1)oc1ccccc13)OCCO2)c1cc(C(=N)N)cs1]

### Step 1: 3-bromodibenzo[b,d]furan (2)

To a solution of dibenzo[b,d]furan-3-amine (2 g, 10.92 mmol) in MeCN (50 mL) was added tert-butyl nitrite (1.1 g, 10.92 mmol) and CuBr (1.9 g, 13.10 mmol) and the reaction mixture was stirred at 90° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give 3-bromodibenzo[b,d]furan (430 mg, yield 15.9%) as a white solid. LC/MS (ESI) m/z:247 (M+H)⁺.

### Step 2: methyl dibenzo[b,d]furan-3-carboxylate (3)

To a solution of 3-bromodibenzo[b,d]furan (400 mg, 1.62 mmol) in MeOH (10 mL) was added TEA (491 mg, 4.86 mmol) and Pd(dppf)Cl₂ (59 mg, 0.081 mmol) and the mixture was degassed under N₂ atmosphere for three times and stirred under a CO balloon at 80° C. overnight. The mixture was diluted with EtOAc and washed with water and brine, dried over anhydrous Na₂SO₄, filtered, and concentrated under reduced pressure to dryness. The residue was purified by flash chromatography (silica gel, 0-25% EtOAc in PE) to give methyl dibenzo[b,d]furan-3-carboxylate (310 mg, yield 84.6%) as a white solid. LC/MS (ESI) m/z:227 (M+H)⁺.

### Step 3: dibenzo[b,d]furan-3-carboxylic acid (4)

To a solution of methyl dibenzo[b,d]furan-3-carboxylate (310 mg, 1.37 mmol) in THF/MeOH/H₂O (6 mL,4/1/1) was added LiOH (115 mg, 2.74 mmol) under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give dibenzo[b,d]furan-3-carboxylic acid (290 mg, yield 99.7%) as a white solid. LC/MS (ESI) m/z:213 (M+H)⁺.

### Step 4: methyl (dibenzo[b,d]furan-3-carbonyl)glycinate (5)

To a mixture of dibenzo[b,d]furan-3-carboxylic acid (290 mg, 1.37 mmol) and methyl glycinate (183 mg, 2.05 mmol) in DMF (10 mL) was added HATU (779 mg, 2.05 mmol) and DIPEA (1.1 g, 8.20 mmol) under N₂ atmosphere and the reaction mixture was stirred at room temperature for 2 hours. The mixture was diluted with EtOAc and washed with water and brine, dried over anhydrous Na₂SO₄, filtered, and concentrated under reduced pressure to dryness. The residue was purified by flash chromatography (silica gel, 0-18% EtOAc in PE) to give methyl (dibenzo[b,d]furan-3-carbonyl)glycinate (170 mg, yield 43.9%) as a white solid. LC/MS (ESI) m/z:284 (M+H)⁺.

### Step 5: (dibenzo[b,d]furan-3-carbonyl)glycine (6)

To a solution of methyl (dibenzo[b,d]furan-3-carbonyl)glycinate (170 mg, 0.60 mmol) in THF/MeOH/H₂O (6 mL, 4/1/1) was added LiOH H₂O (50 mg, 1.20 mmol) under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give (dibenzo[b,d]furan-3-carbonyl)glycine (110 mg, yield 68.1%) as a white solid. LC/MS (ESI) m/z:270 (M+H)⁺.

### Step 6: methyl (S)-7-((dibenzo[b,d]furan-3-carbonyl)glycyl)-1,4-dioxa-7-azaspiro[4.4]nonane-8-carboxylate (7)

To a mixture of (dibenzo[b,d]furan-3-carbonyl)glycine (110 mg, 0.41 mmol) and methyl (S)-1,4-dioxa-7-azaspiro[4.4]nonane-8-carboxylate (76 mg, 0.41 mmol) in DMF (5 mL) was added DIPEA (317 mg, 2.45 mmol) and T₃P (390 mg, 0.61 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-25% EtOAc in PE) to give methyl (S)-7-((dibenzo[b,d]furan-3-carbonyl)glycyl)-1,4-dioxa-7-azaspiro [4.4]nonane-8-carboxylate (100 mg, yield 55.8%) as a white solid. LC/MS (ESI) m/z: 439 (M+H)⁺.

### Step 7: (S)-7-((dibenzo[b,d]furan-3-carbonyl)glycyl)-1,4-dioxa-7-azaspiro[4.4]nonane-6-carboxylic acid (8)

To a solution of methyl (S)-7-((dibenzo[b,d]furan-3-carbonyl)glycyl)-1,4-dioxa-7-azaspiro[4.4]nonane-8-carboxylate (100 mg, 0.23 mmol) in THF/MeOH/H₂O (6 mL,4/1/1) was added LiOH·H₂O (19 mg, 0.46 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give (S)-7-((dibenzo[b,d]furan-3-carbonyl)glycyl)-1,4-dioxa-7-azaspiro[4.4]nonane-8-carboxylic acid (90 mg, yield 93.0%) as a white solid. LC/MS (ESI) m/z:425 (M+H)⁺.

### Step 8: (S)—N—((R)-1-(4-carbamimidoylthiophen-2-yl)ethyl)-7-((dibenzo[b,d]furan-3-carbonyl)glycyl)-1,4-dioxa-7-azaspiro[4.4]nonane-8-carboxamide (Compound 279)

To a mixture of (S)-7-((dibenzo[b,d]furan-3-carbonyl)glycyl)-1,4-dioxa-7-azaspiro[4.4]nonane-8-carboxylic acid (45 mg, 0.11 mmol) and (R)-5-(1-aminoethyl)thiophene-3-carboximidamide (36 mg, 0.21 mmol) in DMF (5 mL) was added DIPEA (82 mg, 0.64 mmol) and T₃P (101 mg, 0.16 mmol, 50% wt. in EtOAc) under N₂ atmosphere and the reaction mixture was stirred at room temperature for 2 hours. The mixture was diluted with EtOAc and washed with saturated aq·NaHCO₃ solution and brine, dried over anhydrous Na₂SO₄, filtered, and concentrated under reduced pressure to dryness. The residue was purified by prep-TLC (DCM:MeOH=5:1) and further purified by prep-HPLC to give Compound 279 (5 mg, yield 8.2%) as a white solid. ¹H NMR (400 MHz, CD₃OD) δ 8.54 (s, 1H), 8.27-8.21 (m, 1H), 8.11 (dd, J=15.4, 6.6 Hz, 3H), 7.93-7.87 (m, 1H), 7.65 (d, J=8.5 Hz, 1H), 7.56 (dd, J=15.1, 7.6 Hz, 2H), 7.43 (t, J=7.3 Hz, 1H), 5.29 (d, J=6.5 Hz, 1H), 4.60 (d, J=3.8 Hz, 1H), 4.24 (d, J=16.6 Hz, 2H), 4.04-3.96 (m, 4H), 3.81 (q, J=10.0 Hz, 2H), 2.44 (dd, J=13.2, 8.4 Hz, 1H), 2.22 (dd, J=13.5, 6.6 Hz, 1H), 1.61 (t, J=18.7 Hz, 3H). LC/MS (ESI) m/z:576 (M+H)⁺.

### Scheme 290: Synthesis of (2S,4R)—N—((R)-1-(4-carbamimidoylthiophen-2-yl)ethyl)-1-((9,9-difluoro-9H-fluorene-3-carbonyl)glycyl)-4-(isopropylsulfonyl)pyrrolidine-2-carboxamide (Compound 280)

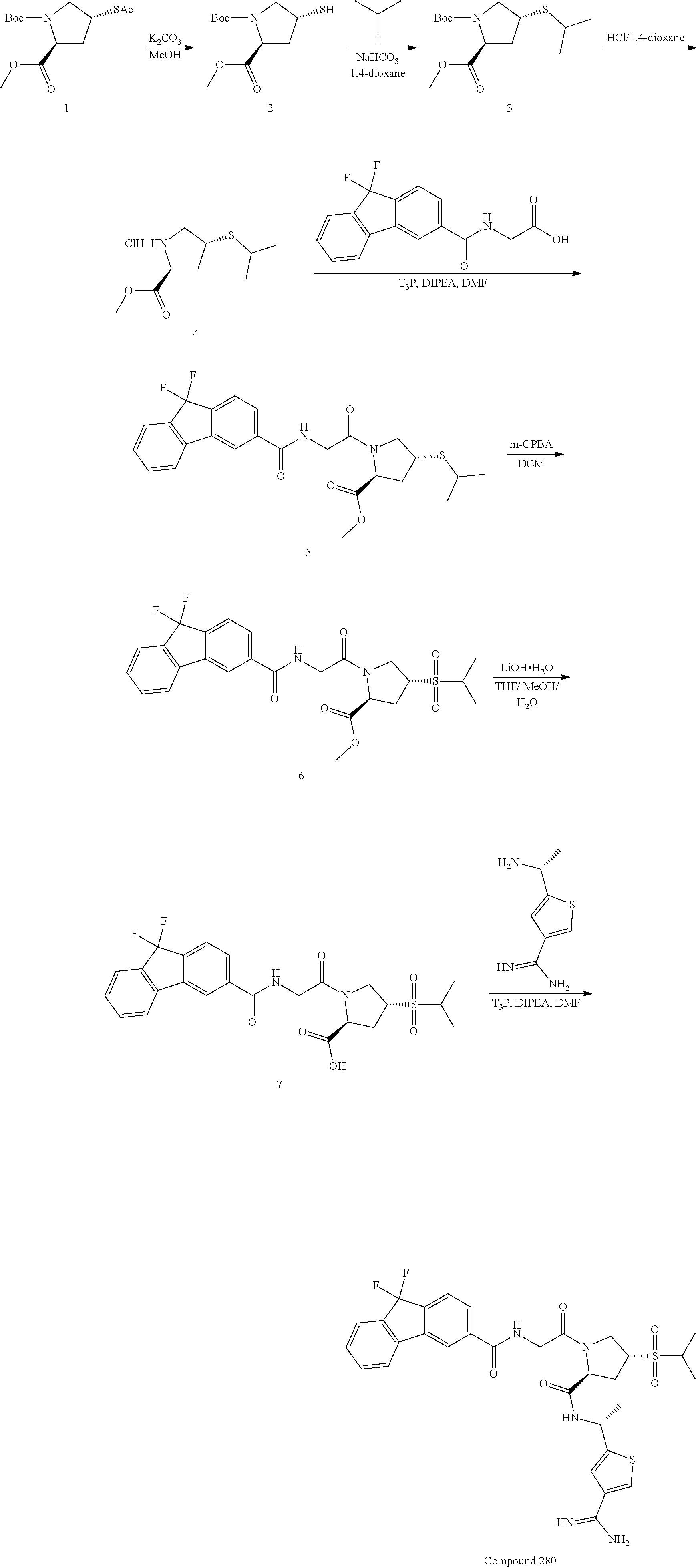

### Step 1: 1-(tert-butyl) 2-methyl (2S,4R)-4-mercaptopyrrolidine-1,2-dicarboxylate (2)

To a solution of 1-(tert-butyl) 2-methyl (2S,4R)-4-(acetylthio)pyrrolidine-1,2-dicarboxylate (2 g, 6.59 mmol) in MeOH/DCM (33 ml, 10/1) was added K₂CO₃ (911 mg, 6.59 mmol) under N₂ atmosphere and the reaction mixture was stirred at room temperature for 2 hours. The mixture was diluted with EtOAc and washed with water and brine, dried over anhydrous Na₂SO₄, filtered, and concentrated under reduced pressure to dryness. The residue was purified by flash chromatography (silica gel, 0-30% EtOAc in PE) to give 1-(tert-butyl) 2-methyl (2S,4R)-4-mercaptopyrrolidine-1,2-dicarboxylate (1.7 g, yield 98.7%) as a white solid. LC/MS (ESI) m/z:262 (M+H)⁺.

### Step 2: 1-(tert-butyl) 2-methyl (2S,4R)-4-(isopropylthio)pyrrolidine-1,2-dicarboxylate (3)

To a solution of 1-(tert-butyl) 2-methyl (2S,4R)-4-mercaptopyrrolidine-1,2-dicarboxylate (1.7 g, 6.51 mmol) in dioxane (20 ml) was added 2-iodopropane (11.4 g, 67.00 mmol) and NaHCO₃ (1.6 g, 19.52 mmol) under N₂ atmosphere and the reaction mixture was stirred at room temperature overnight. The mixture was diluted with EtOAc and washed with water and brine, dried over anhydrous Na₂SO₄, filtered, and concentrated under reduced pressure to dryness. The residue was purified by flash chromatography (silica gel, 0-25% EtOAc in PE) to give 1-(tert-butyl) 2-methyl (2S,4R)-4-(isopropylthio)pyrrolidine-1,2-dicarboxylate (890 mg, yield 45.1%) as a white solid. LC/MS (ESI) m/z:304 (M+H)⁺.

### Step 3: methyl (2S,4R)-4-(isopropylthio)pyrrolidine-2-carboxylate hydrochloride (4)

To a solution of 1-(tert-butyl) 2-methyl (2S,4R)-4-(isopropylthio)pyrrolidine-1,2-dicarboxylate (120 mg, 0.40 mmol) in 1,4-dioxane (2 mL) was added HCl/1,4-dioxane (1 mL) under N₂ atmosphere and the reaction mixture was stirred at room temperature for 3 hours. The mixture was concentrated to dryness under reduced pressure to give methyl (2S,4R)-4-(isopropylthio)pyrrolidine-2-carboxylate hydrochloride (80 mg, yield 84.4%) as a white solid, which was used in next step without purification. LC/MS (ESI) m/z:204 (M+H)⁺.

### Step 4: methyl (2S,4R)-1-((9,9-difluoro-9H-fluorene-3-carbonyl)glycyl)-4-(isopropylthio)pyrrolidine-2-carboxylate (5)

To a mixture of (9,9-difluoro-9H-fluorene-3-carbonyl)glycine (60 mg, 0.20 mmol) and methyl (2S,4R)-4-(isopropylthio)pyrrolidine-2-carboxylate methyl (2S,4R)-4-(isopropylthio)pyrrolidine-2-carboxylate hydrochloride (40 mg, 0.20 mmol) in DMF (5 mL) was added DIPEA (153 mg, 1.19 mmol) and TP (189 mg, 0.30 mmol, 50% wt. in EtOAc) under N₂ atmosphere and the mixture was stirred at room temperature overnight. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-25% EtOAc in PE) to give methyl (2S,4R)-1-((9,9-difluoro-9H-fluorene-3-carbonyl)glycyl)-4-(isopropylthio)pyrrolidine-2-carboxylate (60 mg, yield 62.1%) as a white solid. LC/MS (ESI) m/z: 489 (M+H)⁺.

### Step 5: methyl (2S,4R)-1-((9,9-difluoro-9H-fluorene-3-carbonyl)glycyl)-4-(isopropylsulfonyl)pyrrolidine-2-carboxylate (6)

To a solution of methyl (2S,4R)-1-((9,9-difluoro-9H-fluorene-3-carbonyl)glycyl)-4-(isopropylthio)pyrrolidine-2-carboxylate (60 mg, 0.12 mmol) in DCM (3 mL) was added m-CPBA (42 mg, 0.25 mmol) under N₂ atmosphere and the reaction mixture was stirred at room temperature for 3 hours. The mixture was diluted with EtOAc and washed with water and brine, dried over anhydrous Na₂SO₄, filtered, and concentrated under reduced pressure to dryness. The residue was purified by flash chromatography (silica gel, 0-10% MeOH in DCM) to give methyl (2S,4R)-1-((9,9-difluoro-9H-fluorene-3-carbonyl)glycyl)-4-(isopropylsulfonyl)pyrrolidine-2-carboxylate (40 mg, yield 62.6%) as a white solid. LC/MS (ESI) m/z: 521 (M+H)⁺.

### Step 6: (2S,4R)-1-((9,9-difluoro-9H-fluorene-3-carbonyl)glycyl)-4-(isopropylsulfonyl) pyrrolidine-2-carboxylic acid (7)

To a solution of methyl (2S,4R)-1-((9,9-difluoro-9H-fluorene-3-carbonyl)glycyl)-4-(isopropylsulfonyl)pyrrolidine-2-carboxylate (40 mg, 0.077 mmol) in THF/MeOH/H₂O (3 mL,4/1/1) was added LiOH·H₂O (6 mg, 0.15 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to give (2S,4R)-1-((9,9-difluoro-9H-fluorene-3-carbonyl)glycyl)-4-(isopropylsulfonyl)pyrrolidine-2-carboxylic acid (35 mg, yield 90.0%) as a white solid which was used in next step without purification LC/MS (ESI) m/z:507 (M+H)⁺.

### Step 7: (2S,4R)—N—((R)-1-(4-carbamimidoylthiophen-2-yl)ethyl)-1-((9,9-difluoro-9H-fluorene-3-carbonyl)glycyl)-4-(isopropylsulfonyl)pyrrolidine-2-carboxamide (Compound 280)

To a mixture of (2S,4R)-1-((9,9-difluoro-9H-fluorene-3-carbonyl)glycyl)-4-(isopropylsulfonyl)pyrrolidine-2-carboxylic acid (35 mg, 0.069 mmol) and (R)-5-(1-aminoethyl)thiophene-3-carboximidamide (23 mg, 0.14 mmol) in DMF (3 mL) was added DIPEA (54 mg, 0.42 mmol) and T₃P (66 mg, 0.10 mmol, 50% wt. in EtOAc) under N₂ atmosphere and the reaction mixture was stirred at room temperature for 2 hours. The mixture was diluted with EtOAc and washed with saturated aq·NaHCO₃ solution and brine, dried over anhydrous Na₂SO₄, filtered, and concentrated under reduced pressure to dryness. The residue was purified by prep-TLC (DCM:MeOH=5:1) and further purified by prep-HPLC to give Compound 280 (8 mg, yield 17.6%) as a white solid. ¹H NMR (400 MHz, CD₃OD) δ 8.23 (dd, J=16.6, 1.6 Hz, 1H), 8.18 (d, J=7.5 Hz, 1H), 7.90-7.83 (m, 1H), 7.81-7.75 (m, 1H), 7.72 (d, J=8.1 Hz, 1H), 7.66 (d, J=7.4 Hz, 1H), 7.51 (tt, J=24.4, 7.5 Hz, 3H), 5.30 (dq, J=7.1, 6.6 Hz, 1H), 4.75 (ddd, J=13.3, 8.7, 3.9 Hz, 1H), 4.32-4.15 (m, 4H), 4.04 (ddd, J=23.9, 12.8, 5.6 Hz, 1H), 3.40 (ddd, J=16.6, 13.7, 6.8 Hz, 1H), 2.95-2.70 (m, 1H), 2.47 (dddd, J=18.3, 13.6, 7.4, 4.1 Hz, 1H), 1.64 (dd, J=31.1, 7.0 Hz, 3H), 1.39 (td, J=7.2, 3.0 Hz, 6H). LC/MS (ESI) m/z: 658 (M+H)⁺.

### Scheme 291: Synthesis of (1S,3S,5S)-5-((1H-1,2,4-triazol-1-yl)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 281)

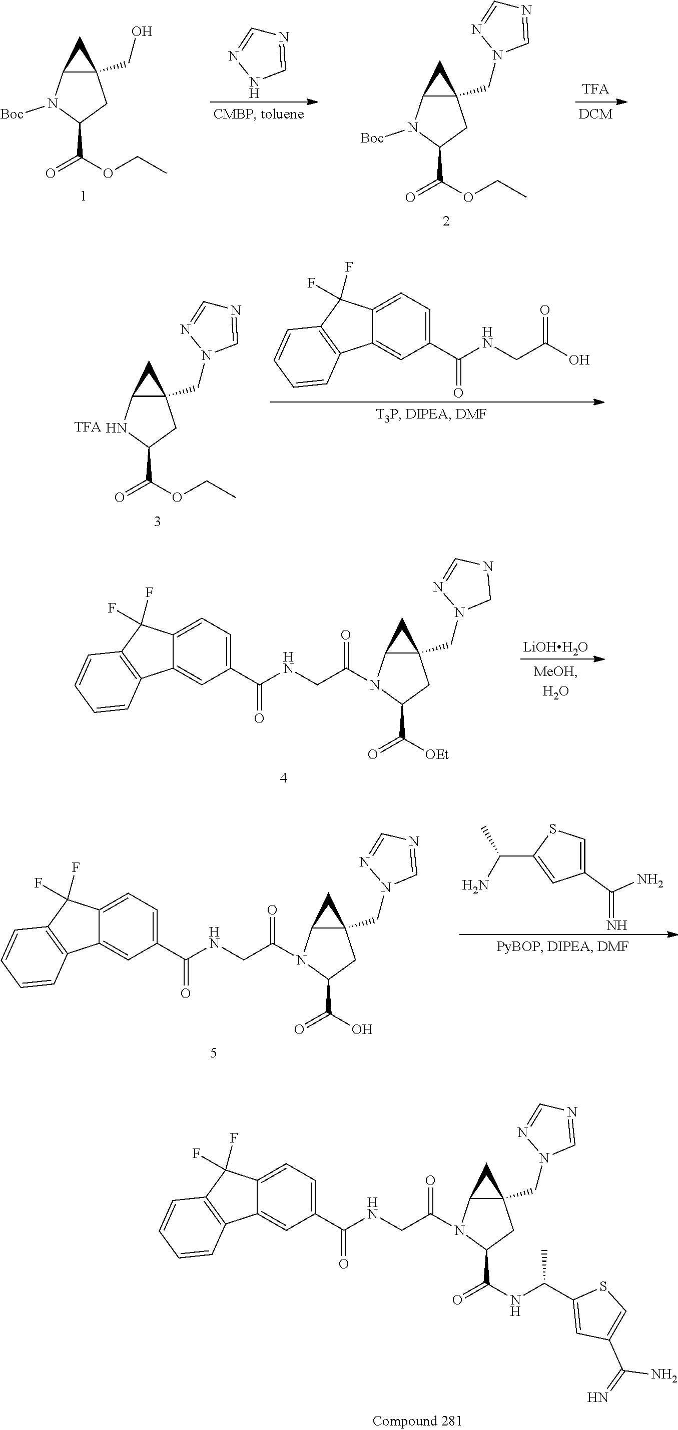

### Step 1: 2-(tert-butyl) 3-ethyl (1S,3S,5S)-5-((1H-1,2,4-triazol-1-yl)methyl)-2-azabicyclo[3.1.0]hexane-2,3-dicarboxylate

To a mixture of 2-(tert-butyl) 3-ethyl (1S,3S,5R)-5-(hydroxymethyl)-2-azabicyclo[3.1.0]hexane-2,3-dicarboxylate (200 mg, 0.70 mmol) and 1H-1,2,4-triazole (97 mg, 1.40 mmol) in toluene (3 mL) was added CMBP (338 mg, 1.40 mmol) under N₂ atmosphere and the reaction mixture was stirred at 100° C. for 16 hours. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give 2-(tert-butyl) 3-ethyl (1S,3S,5S)-5-((1H-1,2,4-triazol-1-yl)methyl)-2-azabicyclo[3.1.0]hexane-2,3-dicarboxylate (120 mg, yield 50.8%) as a yellow oil. LCMS (ESI) m/z: 337 (M+H)⁺.

### Step 2: ethyl (1S,3S,5S)-5-((1H-1,2,4-triazol-1-yl)methyl)-2-azabicyclo[3.1.0]hexane-3-carboxylate 2,2,2-trifluoroacetate

To a solution of 2-(tert-butyl) 3-ethyl (1 S,3S,5S)-5-((1H-1,2,4-triazol-1-yl)methyl)-2-azabicyclo[3.1.0]hexane-2,3-dicarboxylate (120 mg, 0.36 mmol) in DCM (2 mL) was added TFA (1 mL) and the mixture was stirred at room temperature for 1 hour. The mixture was concentrated to dryness under reduced pressure to give ethyl (1S,3S,5S)-5-((1H-1,2,4-triazol-1-yl)methyl)-2-azabicyclo[3.1.0]hexane-3-carboxylate 2,2,2-trifluoroacetate (100 mg, yield 80.0%) as a brown oil, which was directly used in the next reaction without further purification. LC/MS (ESI) m/z: 237 (M+H)⁺.

### Step 3: ethyl (1S,3S,5S)-5-((1H-1,2,4-triazol-1-yl)methyl)-2-((9,9-difluoro-9H-fluorene-3-carbonyl)glycyl)-2-azabicyclo[3.1.0]hexane-3-carboxylate

To a mixture of (9,9-difluoro-9H-fluorene-3-carbonyl)glycine (100 mg, 0.33 mmol) and ethyl (1S,3S,5S)-5-((1H-1,2,4-triazol-1-yl)methyl)-2-azabicyclo[3.1.0]hexane-3-carboxylate 2,2,2-trifluoroacetate (100 mg, 0.43 mmol) in DMF (3 mL) was added T₃P (624 mg, 0.99 mmol, 50% wt. in EtOAc) and DIPEA (0.33 mL, 1.98 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give ethyl (1S,3S,5S)-5-((1H-1,2,4-triazol-1-yl)methyl)-2-((9,9-difluoro-9H-fluorene-3-carbonyl)glycyl)-2-azabicyclo[3.1.0]hexane-3-carboxylate (140 mg, yield 81.4%) as a brown oil. LCMS (ESI) m/z: 522 (M+H)⁺.

### Step 4: (1S,3S,5S)-5-((1H-1,2,4-triazol-1-yl)methyl)-2-((9,9-difluoro-9H-fluorene-3-carbonyl)glycyl)-2-azabicyclo[3.1.0]hexane-3-carboxylic acid

To a solution of ethyl (1S,3S,5S)-5-((1H-1,2,4-triazol-1-yl)methyl)-2-((9,9-difluoro-9H-fluorene-3-carbonyl)glycyl)-2-azabicyclo[3.1.0]hexane-3-carboxylate (140 mg, 0.27 mmol) in MeOH (3.0 mL) and water (1.0 mL) was added LiOH·H₂O (34 mg, 0.41 mmol) at 0° C. and the mixture was stirred at room temperature for 1 hour. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (1S,3S,5S)-5-((1H-1,2,4-triazol-1-yl)methyl)-2-((9,9-difluoro-9H-fluorene-3-carbonyl)glycyl)-2-azabicyclo[3.1.0]hexane-3-carboxylic acid (120 mg, yield 90.9%) as a brown oil, which was used directly in the next reaction. LCMS (ESI) (m/z): 494 (M+H)⁺.

### Step 5: (1S,3S,5S)-5-((1H-1,2,4-triazol-1-yl)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 281)

To a mixture of (1S,3S,5S)-5-((1H-1,2,4-triazol-1-yl)methyl)-2-((9,9-difluoro-9H-fluorene-3-carbonyl)glycyl)-2-azabicyclo[3.1.0]hexane-3-carboxylic acid (80 mg, 0.16 mmol) and (R)-5-(1-aminoethyl)thiophene-3-carboximidamide (67 mg, 0.32 mmol) in DMF (3.0 mL) was added DIPEA (0.16 mL, 0.96 mmol) and PyBOP (101 mg, 0.19 mmol) at 0° C. and the mixture was stirred under N₂ atmosphere at room temperature for 2 hours. The mixture was concentrated to dryness under reduced pressure. The residue was purified by prep-TLC (DCM:MeOH=10:1) and further purified by prep-HPLC to give Compound 281 (15.0 mg, yield 14.3%) as a white solid. ¹H NMR (400 MHz, CD₃OD) δ 8.55 (s, 1H), 8.51 (s, 1H), 8.18 (t, J=1.6 Hz, 2H), 8.03 (s, 1H), 7.88 (d, J=7.8 Hz, 1H), 7.75 (d, J=7.6 Hz, 1H), 7.72 (d, J=7.8 Hz, 1H), 7.65 (d, J=7.3 Hz, 1H), 7.58 (t, J=7.5 Hz, 1H), 7.49-7.43 (m, 2H), 5.20 (q, J=6.8 Hz, 1H), 4.82 (dd, J=11.4, 3.6 Hz, 1H), 4.51 (dd, J=16.9, 8.8 Hz, 1H), 4.41 (dd, J=16.0, 6.4 Hz, 2H), 4.33 (d, J=16.5 Hz, 1H), 3.88 (dd, J=6.2, 2.8 Hz, 1H), 2.60 (t, J=12.1 Hz, 1H), 2.12 (dd, J=13.6, 3.6 Hz, 1H), 1.55 (d, J=7.0 Hz, 3H), 1.47 (dd, J=6.0, 2.8 Hz, 1H), 1.18 (t, J=5.6 Hz, 1H). LCMS (ESI) (m/z): 645 (M+H)⁺.

### Scheme 292: Synthesis of (2S,4R)—N—((R)-1-(4-carbamimidoylthiophen-2-yl)ethyl)-1-((9,9-difluoro-9H-fluorene-3-carbonyl)glycyl)-4-fluoro-4-(hydroxymethyl)pyrrolidine-2-carboxamide (Compound 292)

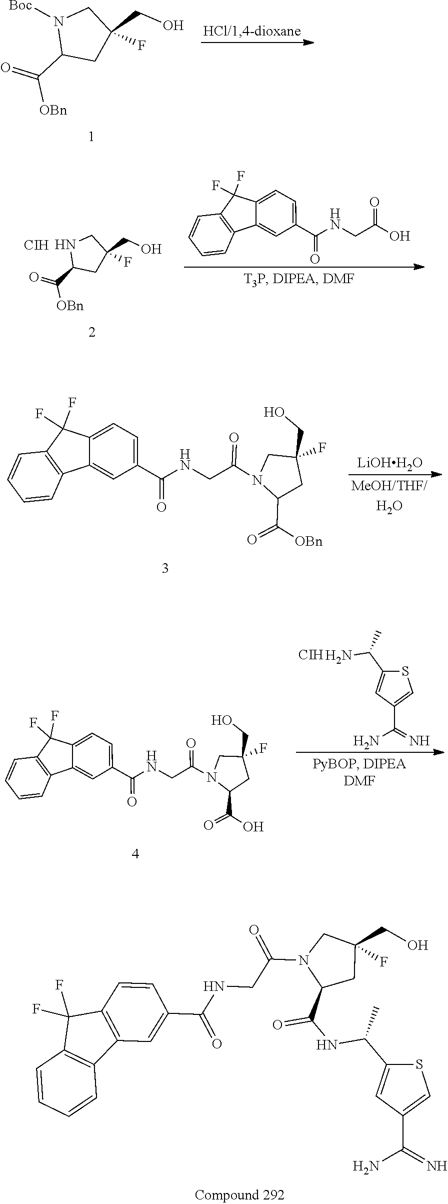

Compound 292 was prepared from 2-benzyl 1-(tert-butyl) (2S,4R)-4-fluoro-4-(hydroxymethyl)pyrrolidine-1,2-dicarboxylate (9,9-difluoro-9H-fluorene-3-carbonyl)glycine, and (R)-5-(1-aminoethyl)thiophene-3-carboximidamide hydrochloride based on the procedures set forth in Scheme 3. ¹H NMR (400 MHz, CD₃OD) δ 8.26-8.15 (m, 2H), 7.89-7.84 (m, 1H), 7.80-7.74 (m, 1H), 7.71 (d, J=7.6 Hz, 1H), 7.65 (d, J=7.2 Hz, 1H), 7.61-7.52 (m, 2H), 7.45 (t, J=7.4 Hz, 1H), 5.41-5.24 (m, 1H), 4.68-4.58 (m, 1H), 4.39-4.31 (m, 1H), 4.20-4.13 (m, 1H), 4.12-4.00 (m, 1H), 3.98-3.86 (m, 1H), 3.86-3.75 (m, 2H), 2.77-2.47 (m, 1H), 2.34-2.11 (m, 1H), 1.62 (dd, J=32.8, 32.8 Hz, 3H). LCMS (ESI) (m/z): 600 (M+H)⁺.

### Scheme 293: Synthesis of(1S,3S,5S)—N-((7-amino-4,5-dihydrothieno[2,3-c]pyridin-2-yl)methyl)-5-methyl-2-((4-phenoxybenzoyl)glycyl)-2-azabicyclo[3.1.0]hexane-3-carboxamide (Compound 293)

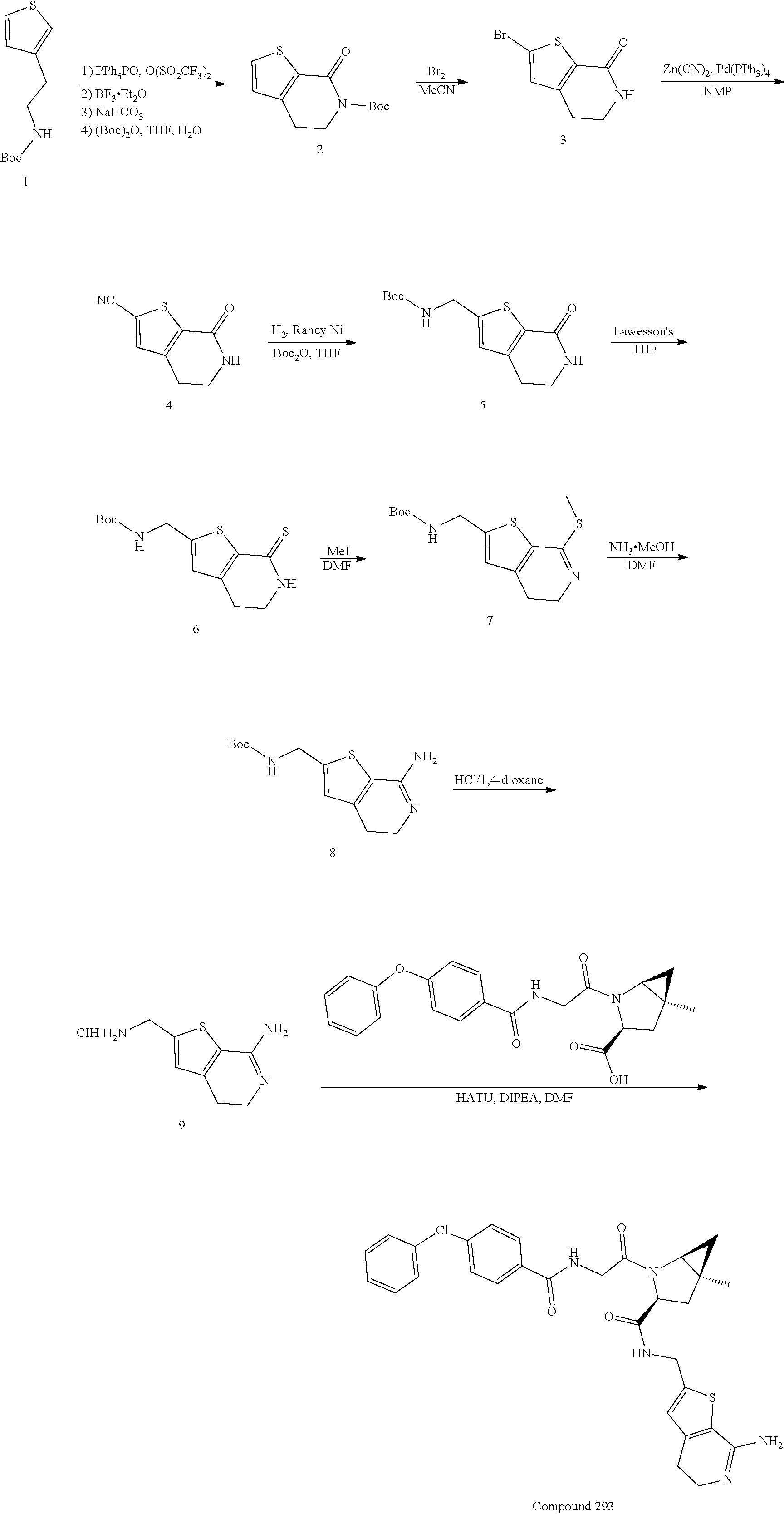

### Step 1: tert-butyl 7-oxo-4,7-dihydrothieno[2,3-c]pyridine-6(5H)-carboxylate

To a solution of PPh₃ (1.91 g, 6.86 mmol) in DCM (10 mL) was added Tf₂O (970 mg, 3.43 mmol) drop-wise at −5° C. and stirred at −5° C. for 0.5 hours under N₂ atmosphere. A solution of tert-butyl (2-(thiophen-3-yl)ethyl)carbamate (650 mg, 2.86 mmol) in DCM (10 mL) was added to the stirring reaction mixture and stirred at −5° C. for 15 minutes, then BF₃-Et₂O (2.03 g, 14.30 mmol) was added and stirred at −5° C. for 1 hours. The mixture was quenched with saturated aq·NaHCO₃ solution and diluted with DCM and washed with brine, dried over anhydrous Na₂SO₄, filtered, and concentrated to dryness. The residue was dissolved in THF (10 mL) was added (Boc)₂O (1.87 g, 8.58 mmol) and DMAP (105 mg, 0.86 mmol), the mixture was stirred at room temperature overnight. The mixture was diluted with water and extracted with EtOAc twice. The combined organic layers were washed with brine, dried with anhydrous Na₂SO₄, filtered, and concentrated to dryness, The residue was purified by flash chromatography (0-12% EtOAc in PE) to give tert-butyl 7-oxo-4,7-dihydrothieno[2,3-c]pyridine-6(5H)-carboxylate (600 mg, yield 82.8%) as a white solid. LC/MS (ESI) (m/z): 254 (M+H)⁺.

### Step 2: 2-bromo-5,6-dihydrothieno[2,3-c]pyridin-7(4H)-one

To a solution of tert-butyl 7-oxo-4,7-dihydrothieno[2,3-c]pyridine-6(5H)-carboxylate (600 mg, 2.37 mmol) in MeCN (6 mL) was added a solution of Br₂(568 mg, 3.55 mmol) in MeCN (2 mL) drop-wise and the mixture was stirred at 0° C. for 8 hours. The mixture was quenched with aq·Na₂S₂O₃ solution and extracted with EtOAc twice. The combined organic layers were washed with brine, dried with anhydrous Na₂SO₄, filtered, and concentrated to dryness, The residue was purified by flash chromatography (0-50% EtOAc in PE) to give 2-bromo-5,6-dihydrothieno[2,3-c]pyridin-7(4H)-one (300 mg, yield 54.6%) as a white solid. LC/MS (ESI) (m/z): 232/234 (M+H)⁺.

### Step 3: 7-oxo-4,5,6,7-tetrahydrothieno[2,3-c]pyridine-2-carbonitrile

To a solution of 2-bromo-5,6-dihydrothieno[2,3-c]pyridin-7(4H)-one (100 mg, 0.43 mmol) in NMP (3 mL) was added Zn(CN)₂ (127 mg, 1.08 mmol) and Pd(PPh₃)₄ at 0° C., and the mixture was stirred at 120° C. for 6 hours. The mixture was diluted with water and extracted with EtOAc twice. The combined organic layers were washed with brine, dried with anhydrous Na₂SO₄, filtered, and concentrated to dryness. The residue was purified by flash chromatography (0-60% EtOAc in PE) to give 7-oxo-4,5,6,7-tetrahydrothieno[2,3-c]pyridine-2-carbonitrile (65 mg, yield 84.7%) as a light oil. LC/MS (ESI) m/z: 179 (M+H)⁺.

### Step 4: tert-butyl ((7-oxo-4,5,6,7-tetrahydrothieno[2,3-c]pyridin-2-yl)methyl) carbamate

To a solution of 7-oxo-4,5,6,7-tetrahydrothieno[2,3-c]pyridine-2-carbonitrile (65 mg, 0.37 mmol) in THF (3 mL) was added (Boc)₂O (159 mg, 0.73 mmol) at 0° C. followed by Raney Ni (30 mg) and the reaction mixture was degassed under N₂ atmosphere for three times and stirred under a H₂ balloon at room temperature for 4 hours. The mixture was filtered and concentrated to dryness under reduced pressure. The residue was purified by pre-TLC (DCM:MeOH=20:1) to give tert-butyl ((7-oxo-4,5,6,7-tetrahydrothieno[2,3-c]pyridin-2-yl)methyl)carbamate (49 mg, yield 47.6%) as a light oil. LC/MS (ESI) m/z: 283 (M+H)⁺.

### Step 5: tert-butyl ((7-thioxo-4,5,6,7-tetrahydrothieno[2,3-c]pyridin-2-yl)methyl) carbamate

To a solution of tert-butyl ((7-oxo-4,5,6,7-tetrahydrothieno[2,3-c]pyridin-2-yl)methyl)carbamate (49 mg, 0.17 mmol) in THF(3 mL) was added Lawesson's Reagent (140 mg, 0.35 mmol) and the mixture was stirred at room temperature for 3 hours. The mixture was concentrated to dryness. The residue was purified by prep-TLC (DCM:MeOH=20:1) to give tert-butyl ((7-thioxo-4,5,6,7-tetrahydrothieno[2,3-c]pyridin-2-yl)methyl)carbamate (25 mg, yield 48.3%) as a light oil. LC/MS (ESI) m/z: 299 (M+H)⁺.

### Step 6: tert-butyl ((7-(methylthio)-4,5-dihydrothieno[2,3-c]pyridin-2-yl)methyl) carbamate

To a solution of tert-butyl ((7-thioxo-4,5,6,7-tetrahydrothieno[2,3-c]pyridin-2-yl)methyl)carbamate (25 mg, 0.084 mmol) in DMF (2 mL) was added MeI (0.4 mL) and the mixture was stirred at 0° C. for 2 hours. The mixture was concentrated to dryness under reduced pressure to give tert-butyl ((7-(methylthio)-4,5-dihydrothieno[2,3-c]pyridin-2-yl)methyl)carbamate (26 mg, yield 99.3%) as a yellow oil, which was directly used in the next reaction without further purification. LC/MS (ESI) m/z: 313 (M+H)⁺.

### Step 7: tert-butyl ((7-amino-4,5-dihydrothieno[2,3-c]pyridin-2-yl)methyl)carbamate

To a solution of tert-butyl ((7-(methylthio)-4,5-dihydrothieno[2,3-c]pyridin-2-yl)methyl)carbamate (26 mg, 0.083 mmol) in DMF (2 mL) was added NH₃·MeOH (1 mL, 7 M), the mixture was stirred at 50° C. for 24 hours. The mixture was concentrated to dryness. The residue was purified by prep-TLC (DCM:MeOH=10:1) to give tert-butyl ((7-amino-4,5-dihydrothieno[2,3-c]pyridin-2-yl)methyl)carbamate (20 mg, yield 85.4%) as a light oil. LC/MS (ESI) m/z: 282 (M+H)⁺.

### Step 6: 2-(aminomethyl)-4,7-dihydrothieno[2,3-c]pyridin-7-amine hydrochloride

A solution of tert-butyl ((7-amino-4,5-dihydrothieno[2,3-c]pyridin-2-yl)methyl) carbamate (20 mg, 0.071 mmol) in HCl/1,4-dioxane (2 mL, 4 M) was stirred under N₂ atmosphere at room temperature for 4 hours. The reaction mixture was concentrated to dryness under reduced pressure, co-evaporated with DCM and dried under vacuum to give 2-(aminomethyl)-4,7-dihydrothieno[2,3-c]pyridin-7-amine hydrochloride (15 mg, yield 96.9%) as a yellow solid, which was used directly in the next reaction without further purification. LC/MS (ESI) m/z: 182 (M+H)⁺.

### Step 9: (1S,3S,5S)—N-((7-amino-4,5-dihydrothieno[2,3-c]pyridin-2-yl)methyl)-5-methyl-2-((4-phenoxybenzoyl)glycyl)-2-azabicyclo[3.1.0]hexane-3-carboxamide (Compound 293)

To a mixture of 2-(aminomethyl)-4,7-dihydrothieno[2,3-c]pyridin-7-amine hydrochloride (15 mg, 0.069 mmol) and (1S,3S,5S)-5-methyl-2-((4-phenoxybenzoyl) glycyl)-2-azabicyclo[3.1.0]hexane-3-carboxylic acid (27 mg, 0.069 mmol) in DMF (2 mL) was added DIPEA (45 mg, 0.68 mmol) and HATU (31 mg, 0.083 mmol) at 0° C. and the mixture was stirred under N₂ atmosphere at room temperature for 1 hour. The mixture was diluted with EtOAc, washed with water and brine, dried over anhydrous Na₂SO₄, filtered, and concentrated to dryness under reduced pressure. The residue was purified by prep-TLC (DCM:MeOH=10:1) and further purified by prep-HPLC to give Compound 293 (3.0 mg, yield 7.8%) as a white solid. ¹H NMR (400 MHz, CD₃OD) δ 7.88-7.78 (m, 2H), 7.43 (t, J=8.0 Hz, 2H), 7.21 (t, J=7.6 Hz, 1H), 7.11-7.02 (m, 3H), 7.02-6.95 (m, 2H), 4.84-4.81 (m, 1H), 4.68-4.57 (m, 2H), 4.44-4.27 (m, 2H), 3.61-3.53 (m, 2H), 3.46-3.36 (m, 1H), 3.00-2.86 (m, 2H), 2.59-2.39 (m, 1H), 2.24-2.05 (m, 1H), 1.31 (s, 3H), 1.19-0.99 (m, 1H), 0.92-0.79 (m, 1H).LC/MS (ESI) m/z: 558 (M+H)⁺.

### Scheme 294: Synthesis of N-(2-((S)-8-(((R)-1-(4-carbamimidoylthiophen-2-yl)ethyl) carbamoyl)-1,4-dioxa-7-azaspiro[4.4]nonan-7-yl)-2-oxoethyl)-10H-phenoxazine-3-carboxamide (Compound 294)

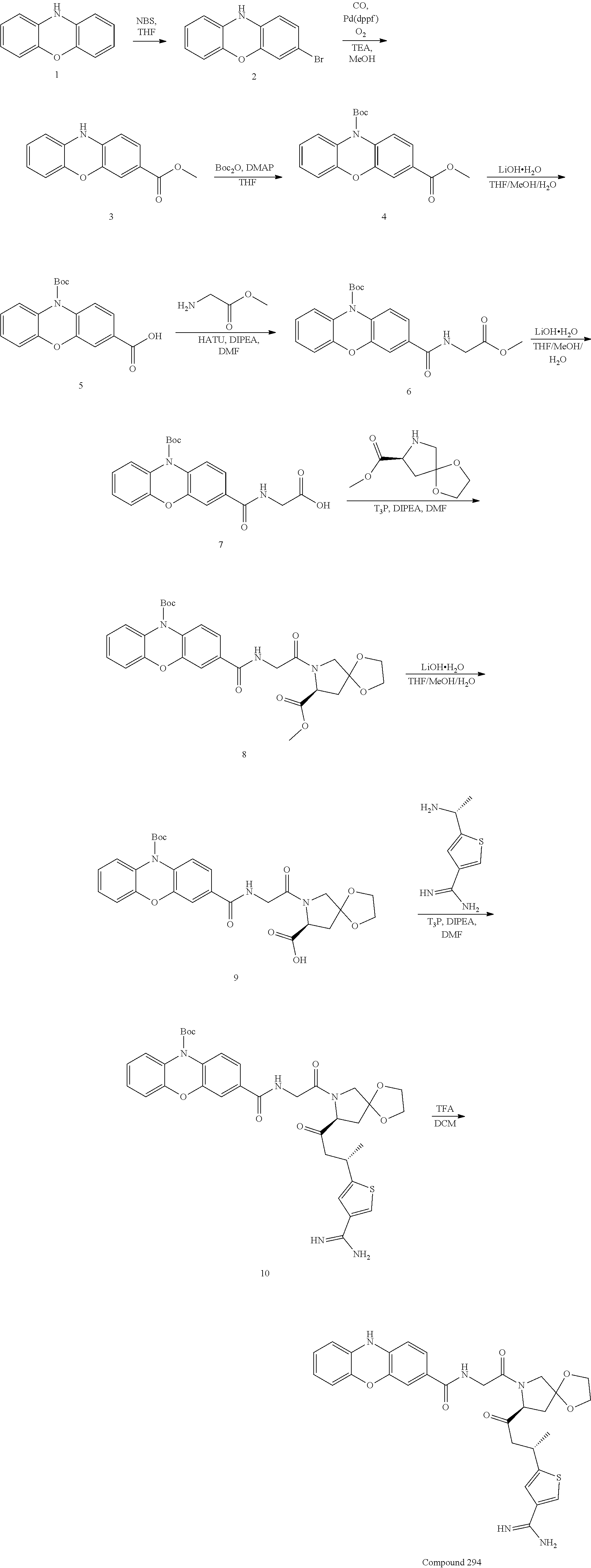

### Step 1: 3-bromo-10H-phenoxazine (2)

To a solution of 10H-phenoxazine (2 g, 10.92 mmol) in THE (30 mL) was added NBS (1.9 g, 10.92 mmol) and the reaction mixture was stirred at 0° C. for 2 hours. The mixture was diluted with EtOAc and washed with water and brine, dried over anhydrous Na₂SO₄, filtered, and concentrated under reduced pressure to dryness. The residue was purified by flash chromatography (silica gel, 0-10% EtOAc in PE) to give 3-bromo-10H-phenoxazine (1.5 g, yield 52.4%) as a white solid. LC/MS (ESI) m/z:262 (M+H)⁺.

### Step 2: methyl 10H-phenoxazine-3-carboxylate (3)

To a solution of 3-bromo-10H-phenoxazine (1.5 g, 5.72 mmol) in MeOH (30 mL) was added TEA (1.7 g, 17.17 mmol) and Pd(dppf)Cl₂ (209 mg, 0.29 mmol) and the reaction mixture was stirred under a CO balloon at 80° C. overnight. The mixture was diluted with EtOAc and washed with water and brine, dried over anhydrous Na₂SO₄, filtered, and concentrated under reduced pressure to dryness. The residue was purified by flash chromatography (silica gel, 0-30% EtOAc in PE) to give methyl 10H-phenoxazine-3-carboxylate (200 mg, yield 14.5%) as a white solid. LC/MS (ESI) m/z:242 (M+H)⁺.

### Step 3: 10-(tert-butyl) 3-methyl 10H-phenoxazine-3,10-dicarboxylate (4)

To a solution of methyl 10H-phenoxazine-3-carboxylate (200 mg, 0.83 mmol) in THE (10 ml) was added Boc₂O (905 mg, 4.15 mmol) and DMAP (10 mg, 4.15 mmol) under N₂ atmosphere and the reaction mixture was stirred at room temperature for 3 hours. The mixture was diluted with EtOAc and washed with water and brine, dried over anhydrous Na₂SO₄, filtered, and concentrated under reduced pressure to dryness. The residue was purified by flash chromatography (silica gel, 0-30% EtOAc in PE) to give 10-(tert-butyl) 3-methyl 10H-phenoxazine-3,10-dicarboxylate (200 mg, yield 70.7%) as a white solid. LC/MS (ESI) m/z:342 (M+H)⁺.

### Step 4: 10-(tert-butoxycarbonyl)-10H-phenoxazine-3-carboxylic acid (5)

To a solution of 10-(tert-butyl) 3-methyl 10H-phenoxazine-3,10-dicarboxylate (200 mg, 0.59 mmol) in THF/MeOH/H₂O (6 mL,4/1/1) was added LiOH·H₂O (49 mg, 1.17 mmol) under N₂ atmosphere and the reaction mixture was stirred at room temperature for 2 hours. The mixture was acidified with 1 N aq·HCl to pH˜3 and extracted with EtOAc twice. The combined organic layers were washed with brine, dried over anhydrous Na₂SO₄, filtered, and concentrated under reduced pressure to give 10-(tert-butoxycarbonyl)-10H-phenoxazine-3-carboxylic acid (190 mg, yield 99.1%) as a white solid, which was used in next step without purification. LC/MS (ESI) m/z:328 (M+H)⁺.

### Step 5: tert-butyl 3-((2-methoxy-2-oxoethyl)carbamoyl)-10H-phenoxazine-10-carboxylate (6)

To a mixture of 10-(tert-butoxycarbonyl)-10H-phenoxazine-3-carboxylic acid (190 mg, 0.58 mmol) and methyl glycinate (78 mg, 0.87 mmol) in DMF (10 mL) was added HATU (331 mg, 0.87 mmol) and DIPEA (450 mg, 3.48 mmol) and the reaction mixture was stirred at room temperature for 2 hours. The mixture was diluted with EtOAc and washed with water and brine, dried over anhydrous Na₂SO₄, filtered, and concentrated under reduced pressure to dryness. The residue was purified by flash chromatography (silica gel, 0-20% EtOAc in PE) to give tert-butyl 3-((2-methoxy-2-oxoethyl)carbamoyl)-10H-phenoxazine-10-carboxylate (210 mg, yield 90.8%) as a white solid. LC/MS (ESI) m/z:399 (M+H)⁺.

### Step 6: (10-(tert-butoxycarbonyl)-10H-phenoxazine-3-carbonyl)glycine (7)

To a solution of tert-butyl 3-((2-methoxy-2-oxoethyl)carbamoyl)-10H-phenoxazine-10-carboxylate (210 mg, 0.53 mmol) in THF/MeOH/H₂O (6 mL,4/1/1) was added LiOH·H₂O (44 mg, 1.05 mmol) and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give (10-(tert-butoxycarbonyl)-10H-phenoxazine-3-carbonyl)glycine (190 mg, yield 93.8%) as a white solid. LC/MS (ESI) m/z:385 (M+H)⁺.

### Step 7: tert-butyl (S)-3-((2-(8-(methoxycarbonyl)-1,4-dioxa-7-azaspiro[4.4]nonan-7-yl)-2-oxoethyl)carbamoyl)-10H-phenoxazine-10-carboxylate (8)

To a mixture of (10-(tert-butoxycarbonyl)-10H-phenoxazine-3-carbonyl)glycine (95 mg, 0.25 mmol) and methyl (S)-1,4-dioxa-7-azaspiro[4.4]nonane-8-carboxylate (46 mg, 0.25 mmol) in DMF (5 mL) was added DIPEA (192 mg, 1.48 mmol) and T₃P (236 mg, 0.37 mmol, 50% wt. in EtOAc) and the mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-20% EtOAc in PE) to give tert-butyl (S)-3-((2-(8-(methoxycarbonyl)-1,4-dioxa-7-azaspiro[4.4]nonan-7-yl)-2-oxoethyl)carbamoyl)-10H-phenoxazine-10-carboxylate (90 mg, yield 65.8%) as a white solid. LC/MS (ESI) m/z: 554 (M+H)⁺.

### Step 8: (S)-7-((10-(tert-butoxycarbonyl)-10H-phenoxazine-3-carbonyl)glycyl)-1,4-dioxa-7-azaspiro[4.4]nonane-8-carboxylic acid (9)

To a solution of tert-butyl (S)-3-((2-(8-(methoxycarbonyl)-1,4-dioxa-7-azaspiro [4.4]nonan-7-yl)-2-oxoethyl)carbamoyl)-10H-phenoxazine-10-carboxylate (90 mg, 0.16 mmol) in THF/MeOH/H₂O (3 mL,4/1/1) was added LiOH·H₂O (14 mg, 0.33 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give (S)-7-((10-(tert-butoxycarbonyl)-10H-phenoxazine-3-carbonyl)glycyl)-1,4-dioxa-7-azaspiro [4.4]nonane-8-carboxylic acid (85 mg, yield 96.9%) as a white solid. LC/MS (ESI) m/z:540 (M+H)⁺.

### Step 9: tert-butyl 3-((2-((S)-6-(((R)-1-(4-carbamimidoylthiophen-2-yl)ethyl) carbamoyl)-1,4-dioxa-7-azaspiro[4.4]nonan-7-yl)-2-oxoethyl)carbamoyl)-10H-phenoxazine-10-carboxylate (10)

To a mixture of (S)-7-((10-(tert-butoxycarbonyl)-10H-phenoxazine-3-carbonyl)glycyl)-1,4-dioxa-7-azaspiro[4.4]nonane-8-carboxylic acid (45 mg, 0.083 mmol) and (R)-5-(1-aminoethyl)thiophene-3-carboximidamide (28 mg, 0.17 mmol) in DMF (5 mL) was added DIPEA (65 mg, 0.50 mmol) and T₃P (80 mg, 0.13 mmol, 50% wt. in EtOAc) under N₂ atmosphere and the mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-15% MeOH in DCM) to give tert-butyl 3-((2-((S)-8-(((R)-1-(4-carbamimidoylthiophen-2-yl)ethyl)carbamoyl)-1,4-dioxa-7-azaspiro[4.4]nonan-7-yl)-2-oxoethyl)carbamoyl)-10H-phenoxazine-10-carboxylate (30 mg, yield 52.1%) as a white solid. LC/MS (ESI) m/z: 691 (M+H)⁺.

### Step 10: N-(2-((S)-8-(((R)-1-(4-carbamimidoylthiophen-2-yl)ethyl)carbamoyl)-1,4-dioxa-7-azaspiro[4.4]nonan-7-yl)-2-oxoethyl)-10H-phenoxazine-3-carboxamide (Compound 294)

To a solution of tert-butyl 3-((2-((S)-8-(((R)-1-(4-carbamimidoylthiophen-2-yl)ethyl)carbamoyl)-1,4-dioxa-7-azaspiro[4.4]nonan-7-yl)-2-oxoethyl)carbamoyl)-10H-phenoxazine-10-carboxylate (30 mg, 0.043 mmol) in DCM (2 mL) was added TFA (1 mL) and the reaction mixture was stirred at room temperature for 2 hours. The mixture was concentrated to dryness under reduced pressure and further purified by prep-HPLC to give Compound 294 (4 mg, yield 15.6%) as a white solid. ¹H NMR (400 MHz, CD₃OD) δ 8.24 (dd, J=6.3, 1.6 Hz, 1H), 7.55-7.51 (m, 1H), 7.26 (dd, J=8.2, 1.9 Hz, 1H), 7.04 (d, J=1.9 Hz, 1H), 6.72 (t, J=6.7 Hz, 1H), 6.64-6.56 (m, 2H), 6.43 (t, J=7.7 Hz, 2H), 5.27 (q, J=7.0 Hz, 1H), 4.56 (dd, J=8.8, 6.4 Hz, 1H), 4.09 (s, 2H), 4.02-3.94 (m, 4H), 3.81-3.74 (m, 2H), 2.42 (dd, J=13.2, 9.1 Hz, 1H), 2.20 (dd, J=13.2, 6.5 Hz, 1H), 1.57 (d, J=7.0 Hz, 3H). LC/MS (ESI) m/z:591 (M+H)⁺.

### Scheme 295: Alternative Synthesis of (1S,3S,5R)—N—((R)-1-(4-carbamimidoylthiophen-2-yl)ethyl)-2-((9,9-difluoro-9H-fluorene-3-carbonyl)glycyl)-5-(hydroxymethyl)-2-azabicyclo[3.1.0]hexane-3-carboxamide (Compound 53)

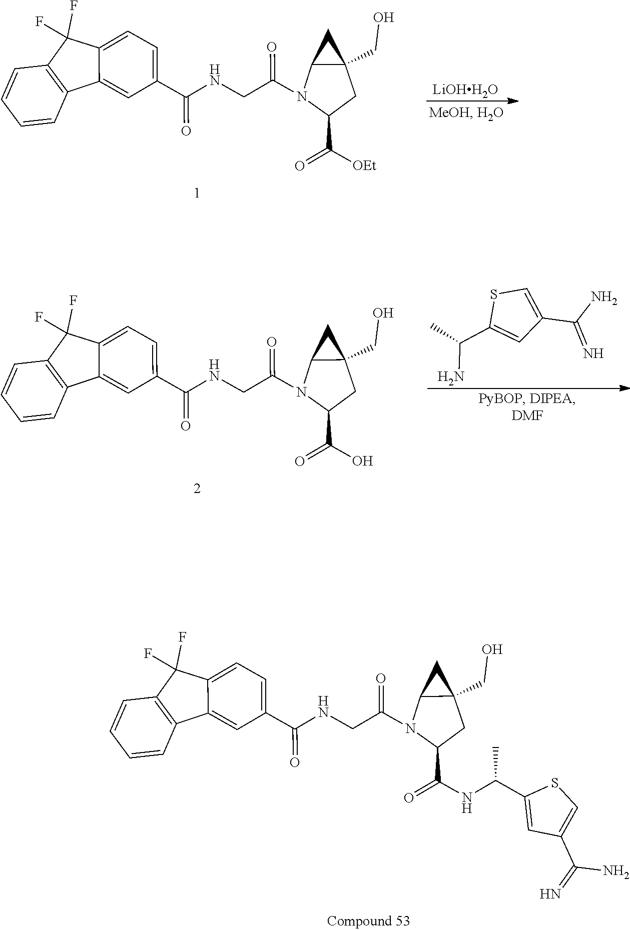 [SMILES: CCOC(=O)[C@@H]1C[C@]2(CO)C[C@@H]2N1C(=O)CNC(=O)c1ccc2c(c1)-c1ccccc1C2(F)F ; "LiOH·H2O" ; O=C(NCC(=O)N1[C@H]2C[C@@]2(CO)C[C@H]1C(=O)O)c1ccc2c(c1)-c1ccccc1C2(F)F ; C[C@@H](N)c1cc(C(=N)N)cs1 ; C[C@@H](NC(=O)[C@@H]1C[C@]2(CO)C[C@@H]2N1C(=O)CNC(=O)c1ccc2c(c1)-c1ccccc1C2(F)F)c1cc(C(=N)N)cs1]

Compound 53 was prepared from ethyl (1 S,3S,5R)-2-((9,9-difluoro-9H-fluorene-3-carbonyl)glycyl)-5-(hydroxymethyl)-2-azabicyclo[3.1.0]hexane-3-carboxylate and (R)-5-(1-aminoethyl)thiophene-3-carboximidamide based on the procedures set forth in Scheme 3. ¹H NMR (400 MHz, CD₃OD) δ 8.53 (s, 1H), 8.20-8.18 (m, 2H), 7.88 (dd, J=7.8, 1.5 Hz, 1H), 7.75 (d, J=7.6 Hz, 1H), 7.72 (d, J=7.9 Hz, 1H), 7.66 (d, J=7.4 Hz, 1H), 7.58 (t, J=7.6 Hz, 1H), 7.51-7.50 (m, 1H), 7.44 (d, J=7.5 Hz, 1H), 5.24 (q, J=6.6 Hz, 1H), 4.89 (d, J=3.7 Hz, 1H), 4.49 (d, J=16.5 Hz, 1H), 4.32 (d, J=16.6 Hz, 1H), 3.68 (d, J=11.7 Hz, 1H), 3.54 (dd, J=11.4, 7.7 Hz, 2H), 2.73 (t, J=12.4 Hz, 1H), 2.11 (dd, J=13.4, 3.7 Hz, 1H), 1.59 (d, J=7.0 Hz, 3H), 1.33 (dd, J=5.6, 2.7 Hz, 1H), 1.01 (t, J=5.4 Hz, 1H). LCMS (ESI) (m/z): 594 (M+H)⁺.

### Scheme 296: Synthesis of (2S,4R*)—N—((R)-1-(4-carbamimidoylthiophen-2-yl)ethyl)-1-((dibenzo[b,d]furan-2-carbonyl)glycyl)-4-(methoxymethyl)pyrrolidine-2-carboxamide (Compound 295)

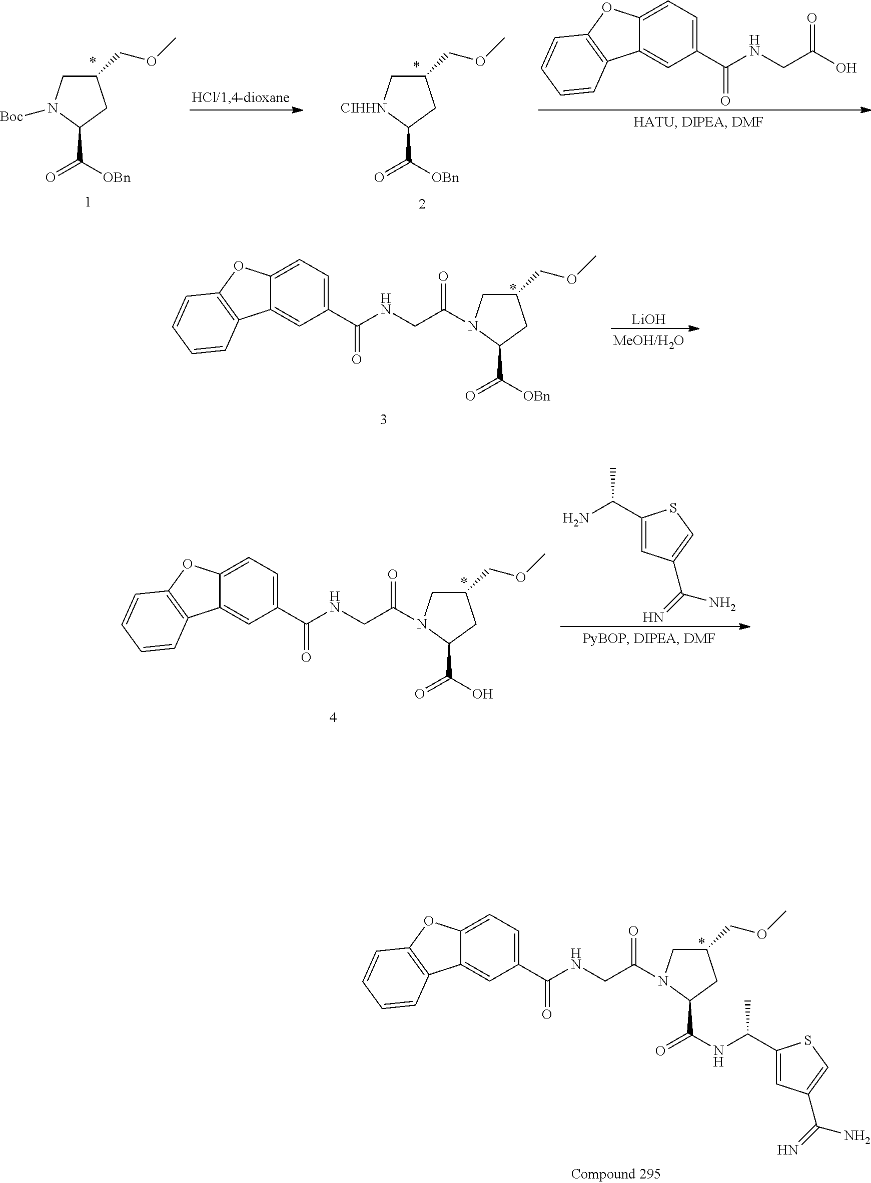

Compound 295 was prepared from 2-benzyl 1-(tert-butyl) (2S,4R*)-4-(methoxymethyl)pyrrolidine-1,2-dicarboxylate, (dibenzo[b,d]furan-2-carbonyl)glycine, and (R)-5-(1-aminoethyl)thiophene-3-carboximidamide based on the procedures set forth in Scheme 3. ¹H NMR (400 MHz, CD₃OD) δ 8.59 (dd, J=7.4, 1.6 Hz, 1H), 8.53 (s, 1H), 8.23 (dd, J=13.6, 1.6 Hz, 1H), 8.08 (d, J=7.7 Hz, 1H), 8.03 (dd, J=8.6, 1.9 Hz, 1H), 7.66 (dd, J=12.6, 8.5 Hz, 2H), 7.55 (dd, J=7.7, 6.6 Hz, 1H), 7.52-7.47 (m, 1H), 7.43 (t, J=7.5 Hz, 1H), 5.29 (t, J=6.5 Hz, 1H), 4.56 (d, J=4.5 Hz, 1H), 4.26 (d, J=3.2 Hz, 2H), 3.93 (dd, J=9.9, 7.6 Hz, 1H), 3.54-3.42 (m, 3H), 3.37 (d, J=8.1 Hz, 3H), 2.84-2.67 (m, 1H), 2.11 (dd, J=10.3, 6.9 Hz, 2H), 1.63 (dd, J=33.7, 7.0 Hz, 3H). LCMS (ESI) (m/z): 562 (M+H)⁺.

### Scheme 297: Synthesis of (1S,3S,5S)-5-(aminomethyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 303)

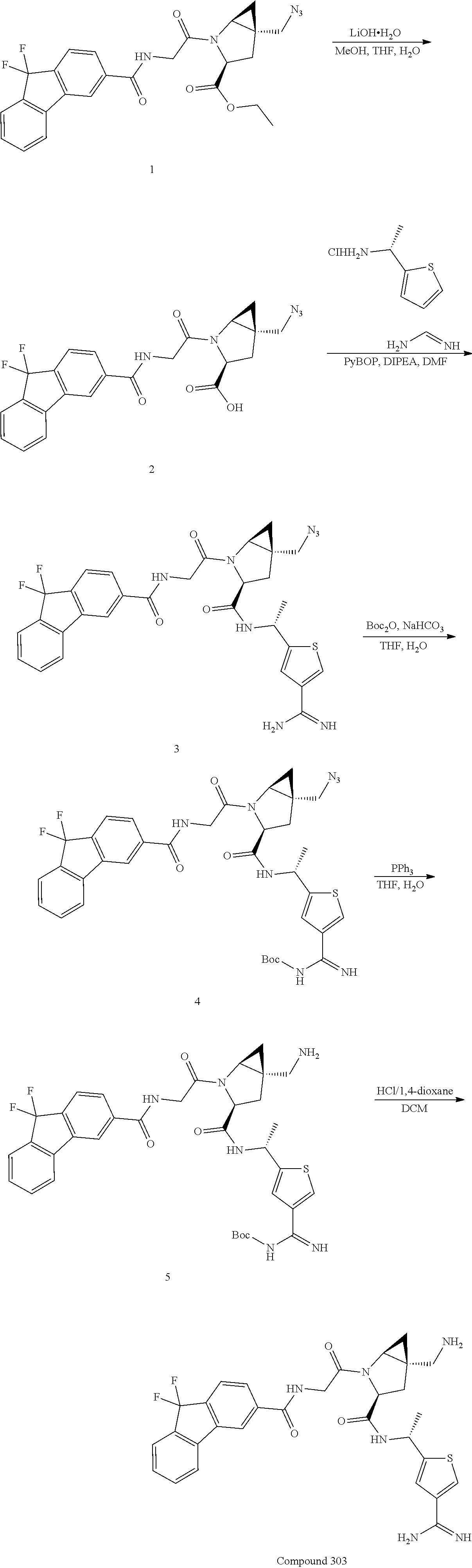

### Step 1: (1S,3S,5S)-5-(azidomethyl)-2-((9,9-difluoro-9H-fluorene-3-carbonyl)glycyl)-2-azabicyclo[3.1.0]hexane-3-carboxylic acid

To a solution of ethyl (1S,3S,5S)-5-(azidomethyl)-2-((9,9-difluoro-9H-fluorene-3-carbonyl)glycyl)-2-azabicyclo[3.1.0]hexane-3-carboxylate (200 mg, 0.41 mmol) in MeOH (2.0 mL) and water (0.6 mL) was added LiOH·H₂O (34 mg, 0.82 mmol) at 0° C. and the mixture was stirred at room temperature for 1 hour. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (1 S,3S,5S)-5-(azidomethyl)-2-((9,9-difluoro-9H-fluorene-3-carbonyl)glycyl)-2-azabicyclo[3.1.0]hexane-3-carboxylic acid (159 mg, yield 83.2%) as a yellow oil, which was used directly in the next reaction. LCMS (ESI) (m/z): 468 (M+H)⁺.

### Step 2: (1S,3S,5S)-5-(azidomethyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide

To a mixture of (1S,3S,5S)-5-(azidomethyl)-2-((9,9-difluoro-9H-fluorene-3-carbonyl) glycyl)-2-azabicyclo[3.1.0]hexane-3-carboxylic acid (159 mg, 0.34 mmol) and (R)-5-(1-aminoethyl)thiophene-3-carboximidamide hydrochloride (88 mg, 0.51 mmol) in DMF (3.0 mL) was added DIPEA (220 mg, 1.70 mmol) and PyBOP (213 mg, 0.41 mmol) at 0° C. and the mixture was stirred under N₂ atmosphere at room temperature for 2 hours. The mixture was concentrated to dryness under reduced pressure give (1S,3S,5S)-5-(azidomethyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (210 mg, crude) as a brown oil, which was used directly in the next reaction. LCMS (ESI) (m/z): 619 (M+H)⁺.

### Step 3: tert-butyl ((5-((R)-1-((1S,3S,5S)-5-(azidomethyl)-2-((9,9-difluoro-9H-fluorene-3-carbonyl)glycyl)-2-azabicyclo[3.1.0]hexane-3-carboxamido)ethyl)thiophen-3-yl)(imino)methyl)carbamate

To a solution of (1 S,3S,5S)-5-(azidomethyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (210 mg, 0.34 mmol) in THE (2.0 mL) and H₂O (0.5 mL) was added Boc₂O (148 mg, 0.68 mmol) and NaHCO₃ (57 mg, 0.68 mmol) and the mixture was stirred at room temperature for 3 hours. The reaction mixture was concentrated to dryness under reduced pressure. The residue was purified by flash chromatography (silica gel, 0-10% MeOH in DCM) to give tert-butyl ((5-((R)-1-((1S,3S,5S)-5-(azidomethyl)-2-((9,9-difluoro-9H-fluorene-3-carbonyl)glycyl)-2-azabicyclo[3.1.0]hexane-3-carboxamido) ethyl)thiophen-3-yl)(imino)methyl)carbamate (200 mg, yield 82.0%) as a yellow oil. LCMS (ESI) m/z: 719 (M+H)⁺.

### Step 4: tert-butyl ((5-((R)-1-((1S,3S,5S)-5-(aminomethyl)-2-((9,9-difluoro-9H-fluorene-3-carbonyl)glycyl)-2-azabicyclo[3.1.0]hexane-3-carboxamido)ethyl) thiophen-3-yl)(imino)methyl)carbamate

To a solution of tert-butyl ((5-((R)-1-((1 S,3S,5S)-5-(azidomethyl)-2-((9,9-difluoro-9H-fluorene-3-carbonyl)glycyl)-2-azabicyclo[3.1.0]hexane-3-carboxamido)ethyl) thiophen-3-yl)(imino)methyl)carbamate (25 mg, 0.035 mmol) in THE (1.0 mL) and H₂O (0.5 mL) was added PPh₃ (14 mg, 0.053 mmol) and the mixture was stirred at room temperature for 3 hours. The reaction mixture was concentrated to dryness under reduced pressure. The residue was purified by prep-TLC (DCM:MeOH=8:1) to give tert-butyl((5-((R)-1-((1S,3S,5S)-5-(aminomethyl)-2-((9,9-difluoro-9H-fluorene-3-carbonyl)glycyl)-2-azabicyclo[3.1.0]hexane-3-carboxamido)ethyl)thiophen-3-yl)(imino) methyl)carbamate (15 mg, yield 62.5%) as a yellow oil. LCMS (ESI) m/z: 693 (M+H)⁺.

### Step 5: (1S,3S,5S)-5-(aminomethyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 303)

To a solution of tert-butyl ((5-((R)-1-((1 S,3S,5S)-5-(aminomethyl)-2-((9,9-difluoro-9H-fluorene-3-carbonyl)glycyl)-2-azabicyclo[3.1.0]hexane-3-carboxamido)ethyl)thiophen-3-yl)(imino)methyl)carbamate (15 mg, 0.022 mmol) in DCM (1.0 mL) was added HCl/1,4-dioxane (1.0 mL, 4 M) and the mixture was stirred at room temperature for 2 hours. The reaction mixture was concentrated to dryness under reduced pressure. The residue was purified by prep-HPLC to give Compound 303 (1.2 mg, yield 9.2%) as a white solid. ¹H NMR (400 MHz, CD₃OD) δ 8.23-8.16 (m, 2H), 7.89 (dd, J=7.8, 1.5 Hz, 1H), 7.75 (dd, J=10.9, 7.8 Hz, 2H), 7.67 (d, J=7.4 Hz, 1H), 7.59 (t, J=7.3 Hz, 1H), 7.51 (dd, J=8.4, 7.2 Hz, 1H), 7.46 (t, J=7.4 Hz, 1H), 5.24 (qd, J=7.0, 1.2 Hz, 1H), 4.94 (dd, J=11.4, 3.5 Hz, 1H), 4.51 (d, J=16.5 Hz, 1H), 4.29 (d, J=16.6 Hz, 1H), 3.81 (dd, J=6.4, 2.9 Hz, 1H), 3.34 (s, 1H), 3.03 (d, J=13.4 Hz, 1H), 2.65 (t, J=12.9 Hz, 1H), 2.29 (dd, J=13.6, 3.5 Hz, 1H), 1.66-1.56 (m, 4H), 1.15 (t, J=6.2 Hz, 1H). LCMS (ESI) (m/z): 593 (M+H)⁺.

### Scheme 298: Synthesis of (1S,3S,5S)—N—((R)-1-(4-carbamimidoylthiophen-2-yl)ethyl)-2-((9,9-difluoro-9H-fluorene-3-carbonyl)glycyl)-5-((dimethylamino)methyl)-2-azabicyclo[3.1.0]hexane-3-carboxamide (Compound 304)

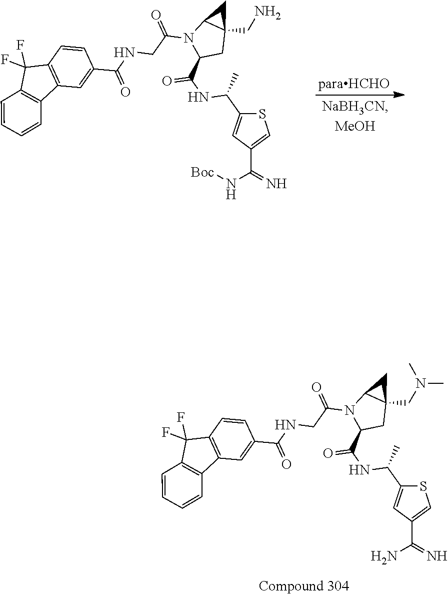 [SMILES: C[C@@H](NC(=O)[C@@H]1C[C@]2(CN)C[C@@H]2N1C(=O)CNC(=O)c1ccc2c(c1)-c1ccccc1C2(F)F)c1cc(C(=N)NC(=O)OC(C)(C)C)cs1 ; C[C@@H](NC(=O)[C@@H]1C[C@]2(CN(C)C)C[C@@H]2N1C(=O)CNC(=O)c1ccc2c(c1)-c1ccccc1C2(F)F)c1cc(C(=N)N)cs1]

To a solution of tert-butyl ((5-((R)-1-((1 S,3S,5S)-5-(aminomethyl)-2-((9,9-difluoro-9H-fluorene-3-carbonyl)glycyl)-2-azabicyclo[3.1.0]hexane-3-carboxamido)ethyl)thiophen-3-yl)(imino)methyl)carbamate (15 mg, 0.022 mmol) in MeOH (1.0 mL) was added Paraformaldehyde (50 mg) and NaBH₃CN (28 mg, 0.44 mmol) and the mixture was stirred at room temperature overnight. The mixture was concentrated to dryness under reduced pressure. The residue was purified by prep-HPLC to give Compound 304 (1.3 mg, yield 9.6%) as a white solid. ¹H NMR (400 MHz, CD₃OD) δ 8.22-8.17 (m, 2H), 7.89 (dd, J=7.8, 1.4 Hz, 1H), 7.75 (dd, J=10.9, 8.2 Hz, 2H), 7.67 (d, J=7.6 Hz, 1H), 7.59 (t, J=7.5 Hz, 1H), 7.51-7.44 (m, 2H), 5.24 (q, J=7.6 Hz, 1H), 4.98 (dd, J=11.2, 3.7 Hz, 1H), 4.51 (d, J=16.5 Hz, 1H), 4.30 (d, J=16.3 Hz, 1H), 3.82 (dd, J=6.6, 3.4 Hz, 1H), 3.72 (d, J=13.6 Hz, 1H), 3.11 (d, J=14.1 Hz, 1H), 2.95 (s, 6H), 2.70 (t, J=12.2 Hz, 1H), 2.36 (dd, J=13.5, 3.5 Hz, 1H), 1.71 (dd, J=6.1, 4.3 Hz, 1H), 1.61 (t, J=14.7 Hz, 3H), 1.29 (t, J=6.1 Hz, 1H). LCMS (ESI) (m/z): 621 (M+H)⁺.

### Scheme 299: Synthesis of (2S,4R*)—N—((R)-1-(4-carbamimidoylthiophen-2-yl)ethyl)-1-((9,9-difluoro-9H-fluorene-3-carbonyl)glycyl)-4-(hydroxymethyl)pyrrolidine-2-carboxamide (Compound 305)

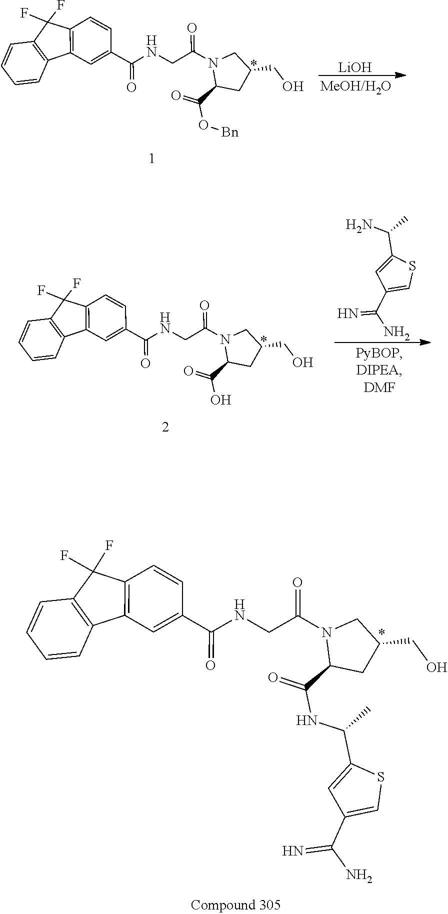 [SMILES: O=C(NCC(=O)N1C[C@H](CO)C[C@H]1C(=O)OCc1ccccc1)c1ccc2c(c1)-c1ccccc1C2(F)F ; "LiOH" ; O=C(NCC(=O)N1C[C@H](CO)C[C@H]1C(=O)O)c1ccc2c(c1)-c1ccccc1C2(F)F ; C[C@@H](N)c1cc(C(=N)N)cs1 ; C[C@@H](NC(=O)[C@@H]1C[C@@H](CO)CN1C(=O)CNC(=O)c1ccc2c(c1)-c1ccccc1C2(F)F)c1cc(C(=N)N)cs1]

Compound 305 was prepared from benzyl (2S,4R*)-1-((9,9-difluoro-9H-fluorene-3-carbonyl)glycyl)-4-(hydroxymethyl)pyrrolidine-2-carboxylate and (R)-5-(1-aminoethyl) thiophene-3-carboximidamide based on the procedures set forth in Scheme 3. ¹H NMR (400 MHz, CD₃OD) δ 8.68 (d, J=7.9 Hz, 1H), 8.24-8.17 (m, 2H), 7.89 (dd, J=7.8, 1.4 Hz, 1H), 7.78-7.71 (m, 2H), 7.65 (d, J=7.5 Hz, 1H), 7.58 (t, J=7.5 Hz, 1H), 7.46 (dd, J=15.1, 7.5 Hz, 2H), 5.33-5.26 (m, 1H), 4.66-4.48 (m, 1H), 4.31-4.21 (m, 2H), 3.90 (dd, J=10.0, 7.7 Hz, 1H), 3.64-3.51 (m, 3H), 2.74-2.59 (m, 1H), 2.09 (dd, J=10.4, 5.8 Hz, 2H), 1.61 (t, J=16.4 Hz, 3H). LCMS (ESI) (m/z): 582 (M+H)⁺.

### Scheme 300: Synthesis of (2S,4S*)—N—((R)-1-(4-carbamimidoylthiophen-2-yl)ethyl)-1-((9,9-difluoro-9H-fluorene-3-carbonyl)glycyl)-4-(hydroxymethyl)pyrrolidine-2-carboxamide (Compound 306)

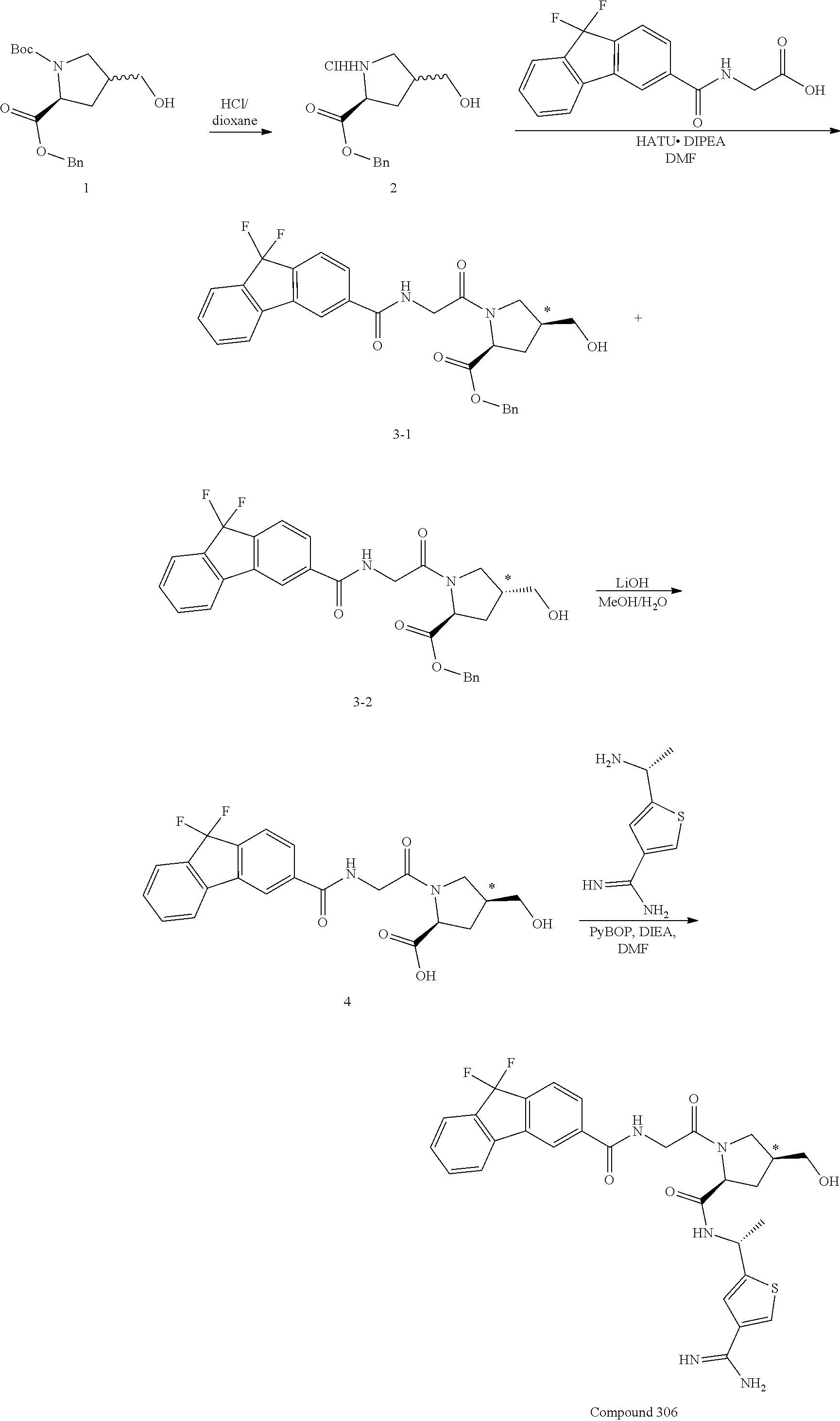

Compound 306 was prepared from 2-benzyl-1-(tert-butyl) (2S)-4-(hydroxymethyl)pyrrolidine-1,2-dicarboxylate, (9,9-difluoro-9H-fluorene-3-carbonyl)glycine, and (R)-5-(1-aminoethyl)thiophene-3-carboximidamide based on the procedures set forth in Scheme 3. ¹H NMR (400 MHz, CD₃OD) δ 8.21 (dd, J=12.2, 10.5 Hz, 2H), 7.87 (dt, J=10.3, 5.2 Hz, 1H), 7.74 (dd, J=17.2, 7.9 Hz, 2H), 7.65 (d, J=7.5 Hz, 1H), 7.60-7.52 (m, 2H), 7.45 (t, J=7.5 Hz, 1H), 5.28 (d, J=7.0 Hz, 1H), 4.49-4.30 (m, 2H), 4.16 (d, J=16.6 Hz, 1H), 3.96 (dd, J=10.0, 7.6 Hz, 1H), 3.63 (t, J=6.0 Hz, 2H), 3.49-3.44 (m, 1H), 2.57 (d, J=7.0 Hz, 1H), 2.47-2.40 (m, 1H), 1.81-1.73 (m, 1H), 1.58 (d, J=7.0 Hz, 3H). LCMS (ESI) (m/z): 582 (M+H)⁺.

### Scheme 301: Synthesis of (2S,4R)—N—((R)-1-(4-carbamimidoylthiophen-2-yl)ethyl)-1-((4-(4-fluorophenoxy)benzoyl)glycyl)-4-(trifluoromethyl)pyrrolidine-2-carboxamide (Compound 307)

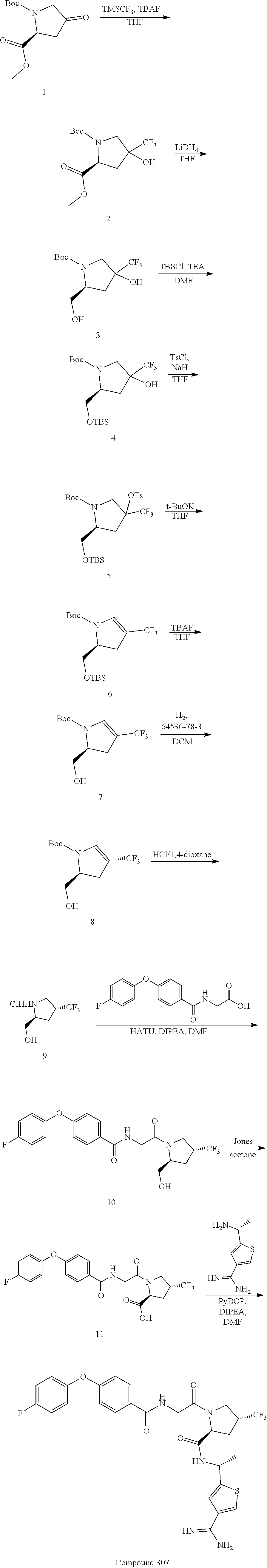

### Step 1: 1-(tert-butyl) 2-methyl (2S)-4-hydroxy-4-(trifluoromethyl)pyrrolidine-1,2-dicarboxylate

To a solution of 1-(tert-butyl) 2-methyl (S)-4-oxopyrrolidine-1,2-dicarboxylate (10 g, 41 mmol) in THE (100 mL) was added TMSCF₃ (12 g, 82 mmol) and TBAF (2 mL, 1 M) under N₂ atmosphere and the reaction solution was stirred at room temperature for 20 hours. The mixture was quenched by drop-wise addition of saturated aq·NH₄Cl solution (50 mL) and TBAF (66 mL, 1 M) at 0° C. The mixture was diluted with EtOAc, and the layers were separated. The organic layers were washed with water and brine, dried over anhydrous Na₂SO₄, filtered, and concentrated under reduced pressure to dryness. The residue was purified by flash chromatography (silica gel, 0-40% EtOAc in PE) to give 1-(tert-butyl) 2-methyl (2S)-4-hydroxy-4-(trifluoromethyl)pyrrolidine-1,2-dicarboxylate (11 g, yield 85.4%) as a yellow oil. LCMS (ESI) m/z: 314 (M+H)⁺.

### Step 2: tert-butyl (2S)-4-hydroxy-2-(hydroxymethyl)-4-(trifluoromethyl)pyrrolidine-1-carboxylate

To a solution of 1-(tert-butyl) 2-methyl (2S)-4-hydroxy-4-(trifluoromethyl)pyrrolidine-1,2-dicarboxylate (5 g, 16 mmol) in THF (50 mL) was added LiBH₄ (18 mL, 1 M in THF) drop-wisely under N₂ atmosphere at 0° C. The mixture was stirred at room temperature for 2 hours. The mixture was quenched with ice-water and extracted with EtOAc twice. The combined organic layers were washed with water and brine, dried over anhydrous Na₂SO₄, filtered, and concentrated under reduced pressure to dryness. The residue was purified by flash chromatography (silica gel, 0-50% EtOAc in PE) to give tert-butyl (2S)-4-hydroxy-2-(hydroxymethyl)-4-(trifluoromethyl)pyrrolidine-1-carboxylate (3 g, yield 65.6%) as a yellow oil. LCMS (ESI) m/z: 286 (M+H)⁺.

### Step 3: tert-butyl (2S)-2-(((tert-butyldimethylsilyl)oxy)methyl)-4-hydroxy-4-(trifluoromethyl)pyrrolidine-1-carboxylate

To a solution of tert-butyl (2S)-4-hydroxy-2-(hydroxymethyl)-4-(trifluoromethyl) pyrrolidine-1-carboxylate (3 g, 10.5 mmol) in DCM (30 mL) was added TBSCl (2.4 g, 16 mmol), TEA (4.2 g, 42 mmol) and DMAP (13 mg, 0.01 mmol) and the mixture was stirred at room temperature overnight. The mixture was diluted with EtOAc, washed with water and brine, dried over anhydrous Na₂SO₄, filtered, and concentrated to dryness under reduced pressure. The residue was purified by flash chromatography (silica gel, 0-20% EtOAc in PE) to give tert-butyl (2S)-2-(((tert-butyldimethylsilyl)oxy)methyl)-4-hydroxy-4-(trifluoromethyl)pyrrolidine-1-carboxylate (2.9 g, yield 69.5%) as a light-white solid. LCMS (ESI) m/z: 400 (M+H)⁺.

### Step 4: tert-butyl (2S)-2-(((tert-butyldimethylsilyl)oxy)methyl)-4-(tosyloxy)-4-(trifluoromethyl)pyrrolidine-1-carboxylate

To a solution of tert-butyl (2S)-2-(((tert-butyldimethylsilyl)oxy)methyl)-4-hydroxy-4-(trifluoromethyl)pyrrolidine-1-carboxylate (2.9 g, 7.3 mmol) in THF (20 mL) was added NaH (455 mg, 11.4 mmol, 60% dispersion in mineral oil) in portions at 0° C. and the mixture was stirred at 0° C. for 30 minutes. A solution of TosCl (2 g, 10.9 mmol) in THF (10 mL) was added drop-wisely to the mixture at room temperature and the resulting mixture was a and stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-10% EtOAc in PE) to give tert-butyl (2S)-2-(((tert-butyldimethylsilyl)oxy)methyl)-4-(tosyloxy)-4-(trifluoromethyl)pyrrolidine-1-carboxylate (3.4 g, yield 83.6%) as a yellow oil. LCMS (ESI) m/z: 554 (M+H)⁺.

### Step 5: tert-butyl (S)-2-(((tert-butyldimethylsilyl)oxy)methyl)-4-(trifluoromethyl)-2,3-dihydro-1H-pyrrole-1-carboxylate

To a solution of tert-butyl (2S)-2-(((tert-butyldimethylsilyl)oxy)methyl)-4-(tosyloxy)-4-(trifluoromethyl)pyrrolidine-1-carboxylate (3.4 g, 6.1 mmol) in THF (30 mL) was added t-BuOK (12 mL, 1 M in THF) drop-wisely at −40° C. under N₂ atmosphere and the mixture was stirred at −40° C. for 1 hour. The mixture was diluted with EtOAc, washed with water and brine, dried over anhydrous Na₂SO₄, filtered, and concentrated under reduced pressure to dryness. The residue was purified by flash chromatography (silica gel, 0-10% EtOAc in PE) to give tert-butyl (S)-2-(((tert-butyldimethylsilyl)oxy)methyl)-4-(trifluoromethyl)-2,3-dihydro-1H-pyrrole-1-carboxylate (1.3 g, yield 55.7%) as a yellow oil. LCMS (ESI) m/z: 382 (M+H)⁺.

### Step 6: tert-butyl (S)-2-(hydroxymethyl)-4-(trifluoromethyl)-2,3-dihydro-1H-pyrrole-1-carboxylate

To a solution of tert-butyl (S)-2-(((tert-butyldimethylsilyl)oxy)methyl)-4-(trifluoromethyl)-2,3-dihydro-1H-pyrrole-1-carboxylate (1 g, 2.6 mmol) in THE (10 mL) was added TBAF (10 mL, 1 M) at 0° C. under N₂ atmosphere and the mixture was stirred at room temperature for 1 hour. The mixture was diluted with EtOAc, washed with saturated aq·NH₄Cl solution and brine, dried over anhydrous Na₂SO₄, filtered, and concentrated under reduced pressure to dryness. The residue was purified by flash chromatography (silica gel, 0-30% EtOAc in PE) to give tert-butyl (S)-2-(hydroxymethyl)-4-(trifluoromethyl)-2,3-dihydro-1H-pyrrole-1-carboxylate (500 mg, yield 73.1%) as a yellow oil. LCMS (ESI) m/z: 268 (M+H)⁺.

### Step 7: tert-butyl (2S,4R)-2-(hydroxymethyl)-4-(trifluoromethyl)pyrrolidine-1-carboxylate

To a solution of tert-butyl (S)-2-(hydroxymethyl)-4-(trifluoromethyl)-2,3-dihydro-1H-pyrrole-1-carboxylate (250 mg, 0.93 mmol) in DCM (5 mL) was added (tricyclohexylphosphine)(1,5-cyclooctadiene)(pyridine)iridium(I) hexafluorophosphate (120 mg, 0.15 mmol) under N₂ atmosphere, the mixture was degassed under N₂ atmosphere for three times and stirred under a H₂ balloon overnight. The mixture was concentrated to dryness and the residue was purified by flash chromatography (silica gel, 0-30% EtOAc in PE) to give tert-butyl (2S,4R)-2-(hydroxymethyl)-4-(trifluoromethyl)pyrrolidine-1-carboxylate (200 mg, yield 79.9%) as orange oil. LCMS (ESI) (m/z): 270 (M+H)⁺.

### Step 8: ((2S,4R)-4-(trifluoromethyl)pyrrolidin-2-yl)methanol hydrochloride

A mixture of tert-butyl (2S,4R)-2-(hydroxymethyl)-4-(trifluoromethyl)pyrrolidine-1-carboxylate (30 mg, 0.25 mmol) in HCl/1,4-dioxane (1 mL) was stirred under N₂ atmosphere at room temperature for 2 hours. The reaction mixture was concentrated to dryness under reduced pressure, co-evaporated with DCM twice and dried under vacuum to give 5-aminothiophene-3-carboximidamide hydrochloride (20 mg, yield 88.7%) as a yellow solid, which was used directly in the next step without further purification. LCMS (ESI) m/z: 170 (M+H)⁺.

### Step 9: 4-(4-fluorophenoxy)-N-(2-((2S,4R)-2-(hydroxymethyl)-4-(trifluoromethyl) pyrrolidin-1-yl)-2-oxoethyl)benzamide

To a mixture of ((2S,4R)-4-(trifluoromethyl)pyrrolidin-2-yl)methanol hydrochloride (20 mg, 0.098 mmol) and (4-(4-fluorophenoxy)benzoyl)glycine (32 mg, 0.11 mmol) in DMF (0.6 mL) was added HATU (50 mg, 0.13 mmol) and DIPEA (85 mg, 0.66 mmol) under N₂ atmosphere and the mixture was stirred at room temperature for 2 hours. The mixture was diluted with EtOAc, washed with water and brine, dried over anhydrous Na₂SO₄, filtered, and concentrated under reduced pressure to dryness. The residue was purified by chromatography (silica gel, 50% EtOAc in PE) to give 4-(4-fluorophenoxy)-N-(2-((2S,4R)-2-(hydroxymethyl)-4-(trifluoromethyl)pyrrolidin-1-yl)-2-oxoethyl)benzamide (36 mg, yield 83.5%) as a yellow oil. LCMS (ESI) m/z: 441 (M+H)⁺.

### Step 10: (2S,4R)-1-((4-(4-fluorophenoxy)benzoyl)glycyl)-4-(trifluoromethyl) pyrrolidine-2-carboxylic acid

To a solution of 4-(4-fluorophenoxy)-N-(2-((2S,4R)-2-(hydroxymethyl)-4-(trifluoromethyl)pyrrolidin-1-yl)-2-oxoethyl)benzamide (35 mg, 0.079 mmol) in acetone (0.5 mL) was added Jone's reagent (4 drops, 2.7 M) at room temperature and the mixture was stirred at room temperature for 10 minutes. The mixture was diluted with EtOAc, washed with water and brine, dried over anhydrous Na₂SO₄, filtered, and concentrated under reduced pressure to dryness. The residue was purified by prep-HPLC to give (2S,4R)-1-((4-(4-fluorophenoxy)benzoyl)glycyl)-4-(trifluoromethyl)pyrrolidine-2-carboxylic acid (20 mg, yield 55.7%) as a white solid. LCMS (ESI) m/z: 455 (M+H)⁺.

### Step 11: Synthesis of (2S,4R)—N—((R)-1-(4-carbamimidoylthiophen-2-yl)ethyl)-1-((4-(4-fluorophenoxy)benzoyl)glycyl)-4-(trifluoromethyl)pyrrolidine-2-carboxamide (Compound 307)

To a mixture of (2S,4R)-1-((4-(4-fluorophenoxy)benzoyl)glycyl)-4-(trifluoromethyl) pyrrolidine-2-carboxylic acid (20 mg, 0.044 mmol) and (R)-5-(1-aminoethyl)thiophene-3-carboximidamide (11 mg, 0.066 mmol) in DMF (0.6 mL) was added PyBOP (25 mg, 0.05 mmol) and DIPEA (34 mg, 0.26 mmol) under N₂ atmosphere and the mixture was stirred at room temperature for 2 hours. The mixture was diluted with EtOAc, washed with water and brine, dried over anhydrous Na₂SO₄, filtered, and concentrated under reduced pressure to dryness. The residue was purified by chromatography (silica gel, 10% MeOH in DCM) and further purified by prep-HPLC to give (2S,4R)—N—((R)-1-(4-carbamimidoylthiophen-2-yl)ethyl)-1-((4-(4-fluorophenoxy)benzoyl)glycyl)-4-(trifluoromethyl)pyrrolidine-2-carboxamide (2 mg, yield 7.5%) as a white solid. ¹H NMR (400 MHz, CD₃OD) δ 8.54 (s, 1H), 8.23 (dd, J=8.6, 1.6 Hz, 1H), 7.87-7.83 (m, 2H), 7.51 (d, J=21.8 Hz, 1H), 7.15 (dd, J=11.5, 5.8 Hz, 2H), 7.09 (dd, J=5.8, 3.4 Hz, 2H), 6.99 (d, J=8.9 Hz, 2H), 5.26 (d, J=6.1 Hz, 1H), 4.65 (s, 1H), 4.26-4.02 (m, 3H), 3.94-3.72 (m, 2H), 3.39 (d, J=8.9 Hz, 1H), 2.45-2.38 (m, 1H), 2.31-2.24 (m, 1H), 1.60 (t, J=15.1 Hz, 3H). LCMS (ESI) m/z: 606 (M+H)⁺.

### Scheme 302: Synthesis of (S)—N—((R)-1-(4-carbamimidoylthiophen-2-yl)ethyl)-7-((phenoxathiine-2-carbonyl)glycyl)-1,4-dioxa-7-azaspiro[4.4]nonane-8-carboxamide (Compound 312)

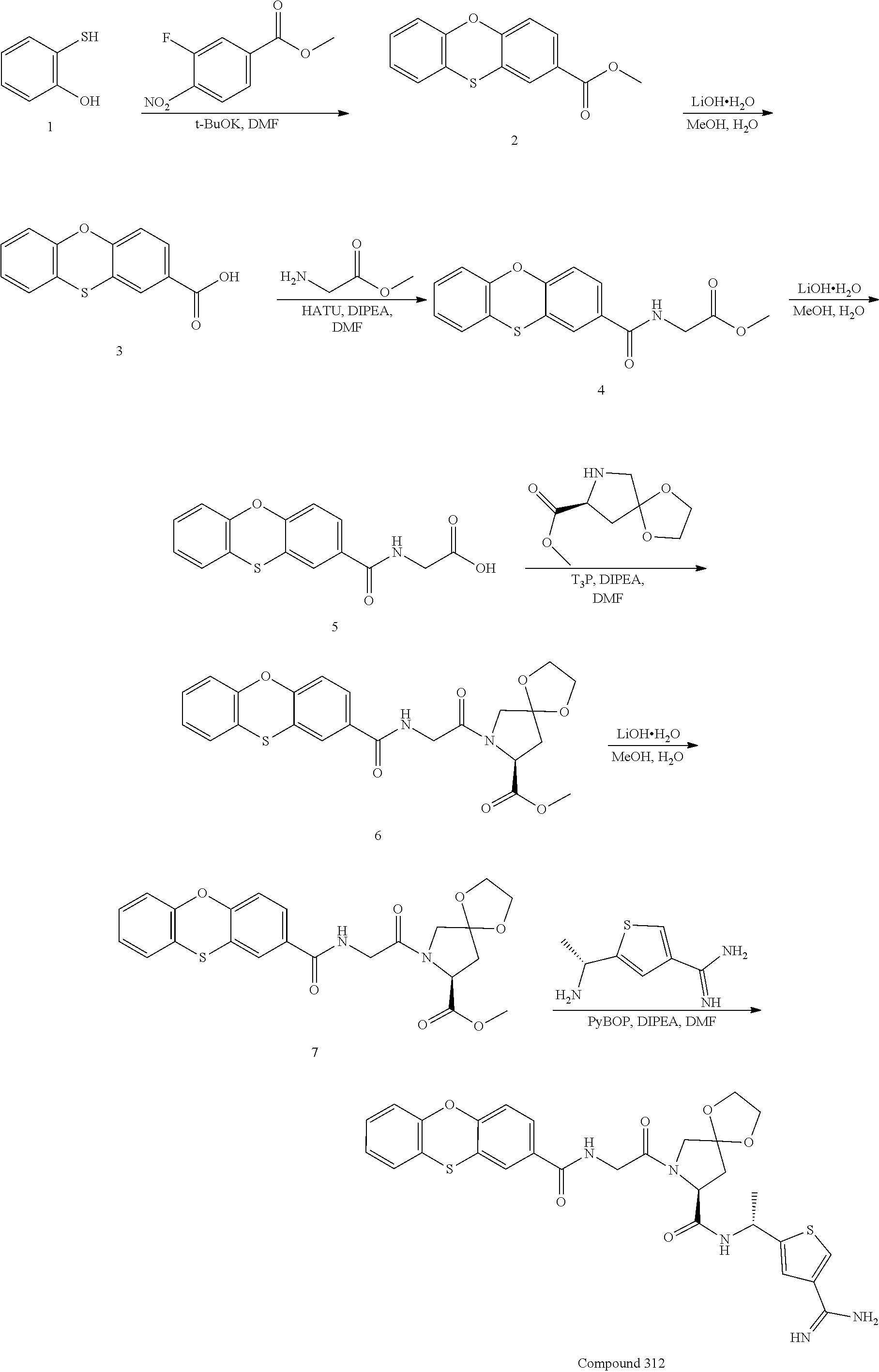

### Step 1: methyl phenoxathiine-2-carboxylate (2)

To a mixture of 2-mercaptophenol (633 mg, 5.02 mmol) and methyl 3-fluoro-4-nitrobenzoate (1 g, 5.02 mmol) in DMF (15 mL) was added t-BuOK (1.13 g, 10.04 mmol) at 0° C. and the mixture was stirred at 160° C. for 8 hours. The mixture was diluted with EtOAc, washed with water and brine, dried over anhydrous Na₂SO₄, filtered, and concentrated to give methyl phenoxathiine-2-carboxylate (1.05 g, yield 81.0%) as a yellow solid. LCMS (ESI) (m/z): 259 (M+H)⁺.

### Step 2: phenoxathiine-3-carboxylic acid (3)

To a solution of methyl phenoxathiine-2-carboxylate (1.05 g, 4.07 mmol) in MeOH (9 mL)/THF (1 mL)/H₂O (3 mL) was added LiOH·H₂O (172 mg, 12.21 mmol) at 0° C. and the mixture was stirred at room temperature for 1 hour. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give phenoxathiine-3-carboxylic acid (870 mg, yield 87.6%) as a yellow solid, which was used directly in the next step without purification. LCMS (ESI) (m/z): 245 (M+H)⁺.

### Step 3: methyl (phenoxathiine-2-carbonyl)glycinate (4)

To a mixture of methyl (phenoxathiine-2-carbonyl)glycinate (870 mg, 3.56 mmol) and methyl glycinate (895 g, 7.13 mmol) in DMF (10 mL) was added DIPEA (2.76 g, 21.36 mmol) and HATU (1.63 g, 4.29 mmol) at 0° C. under N₂ atmosphere and the mixture was stirred at 25° C. for 2 hours. The mixture was diluted with EtOAc and washed with water twice. The combined organic layers were dried over anhydrous Na₂SO₄, filtered, and concentrated under reduced pressure to dryness. The residue was purified by flash chromatography (silica gel, 0-45% EtOAc in PE) to give methyl (phenoxathiine-2-carbonyl)glycinate (870 mg, yield 77.5%) as a yellow solid. LC/MS (ESI) m/z: 316 (M+H)⁺.

### Step 4: (phenoxathiine-2-carbonyl)glycine (5)

To a solution of methyl (phenoxathiine-2-carbonyl)glycinate (870 mg, 2.76 mmol) in MeOH (6 mL)/THF (1 mL)/H₂O (2 mL) was added LiOH·H₂O (348 mg, 8.28 mmol) at 0° C. and the mixture was stirred at room temperature for 1 hour. The mixture was acidified with 1 N aq·HCl to pH˜3 extracted with EtOAc twice. The combined organic layers were washed with brine, dried over anhydrous Na₂SO₄, filtered, and concentrated under reduced pressure to dryness to give (phenoxathiine-2-carbonyl)glycine (760 mg, yield 91.4%) as a white solid, which was used directly in the next step. LCMS (ESI) (m/z): 302 (M+H)⁺.

### Step 5: methyl (S)-7-((phenoxathiine-2-carbonyl)glycyl)-1,4-dioxa-7-azaspiro[4.4]nonane-6-carboxylate (6)

To a mixture of (phenoxathiine-2-carbonyl)glycine (130 mg, 0.43 mmol) and methyl (S)-1,4-dioxa-7-azaspiro[4.4]nonane-8-carboxylate (121 mg, 0.65 mmol) in DMF (3.0 mL) was added DIPEA (334.3 mg, 2.58 mmol) and T₃P (824 mg, 2.58 mmol) at 0° C. under N₂ atmosphere and the mixture was stirred at 25° C. for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-3% MeOH in DCM) to give methyl (S)-7-((phenoxathiine-2-carbonyl)glycyl)-1,4-dioxa-7-azaspiro[4.4]nonane-8-carboxylate (200 mg, yield 98.5%) as a yellow oil. LC/MS (ESI) m/z: 471 (M+H)⁺.

### Step 6: (S)-7-((phenoxathiine-2-carbonyl)glycyl)-1,4-dioxa-7-azaspiro[4.4]nonane-6-carboxylic acid (7)

To a solution of methyl (S)-7-((phenoxathiine-2-carbonyl)glycyl)-1,4-dioxa-7-azaspiro [4.4]nonane-8-carboxylate (200 mg, 0.43 mmol) in MeOH (3 mL) and H₂O (1 mL) was added LiOH·H₂O (53.6 mg, 1.29 mmol) at 0° C. and the mixture was stirred at room temperature for 1 hour. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (S)-7-((phenoxathiine-2-carbonyl)glycyl)-1,4-dioxa-7-azaspiro[4.4]nonane-8-carboxylic acid (180 mg, yield 92.7%) as a yellow solid, which was used directly in the next step. LCMS (ESI) (m/z): 457 (M+H)⁺.

### Step 7: (S)—N—((R)-1-(4-carbamimidoylthiophen-2-yl)ethyl)-7-((phenoxathiine-2-carbonyl)glycyl)-1,4-dioxa-7-azaspiro[4.4]nonane-8-carboxamide (Compound 312)

To a mixture of (S)-7-((phenoxathiine-2-carbonyl)glycyl)-1,4-dioxa-7-azaspiro[4.4]nonane-8-carboxylic acid (80 mg, 0.18 mmol) and (R)-5-(1-aminoethyl)thiophene-3-carboximidamide (60 mg, 0.36 mmol) in DMF (3 mL) was added DIPEA (135 mg, 1.08 mmol) and PyBOP (91.2 mg, 0.18 mmol) at 0° C. and the mixture was stirred under N₂ atmosphere at room temperature for 1 hour. The mixture was concentrated under reduced pressure to dryness. The residue was purified by prep-TLC (DCM:MeOH=10:1) and further purified by prep-HPLC to give Compound 312 (15 mg, yield 14.1%) as a white solid. ¹H NMR (400 MHz, CD₃OD) δ 8.53 (s, 1H), 8.23 (d, J=1.6 Hz, 1H), 7.66 (d, J=8.4 Hz, 1H), 7.64 (d, J=2.0 Hz, 1H), 7.52-7.51 (m, 1H), 7.22-7.18 (m, 1H), 7.14 (d, J=1.6 Hz, 1H), 7.10-7.07 (m, 2H), 7.05 (d, J=1.1 Hz, 1H), 5.27 (q, J=7.4 Hz, 1H), 4.55 (dd, J=8.8, 6.7 Hz, 1H), 4.13 (d, J=3.1 Hz, 2H), 4.00 (s, 2H), 3.99-3.95 (m, 2H), 3.77 (d, J=4.7 Hz, 2H), 2.45-2.39 (m, 1H), 2.20 (dd, J=13.1, 6.6 Hz, 1H), 1.57 (d, J=7.0 Hz, 3H). LCMS (ESI) (m/z): 608 (M+H)⁺.

### Scheme 303: Synthesis of (1S,3S,5R)-5-((2-aminoethoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 313)

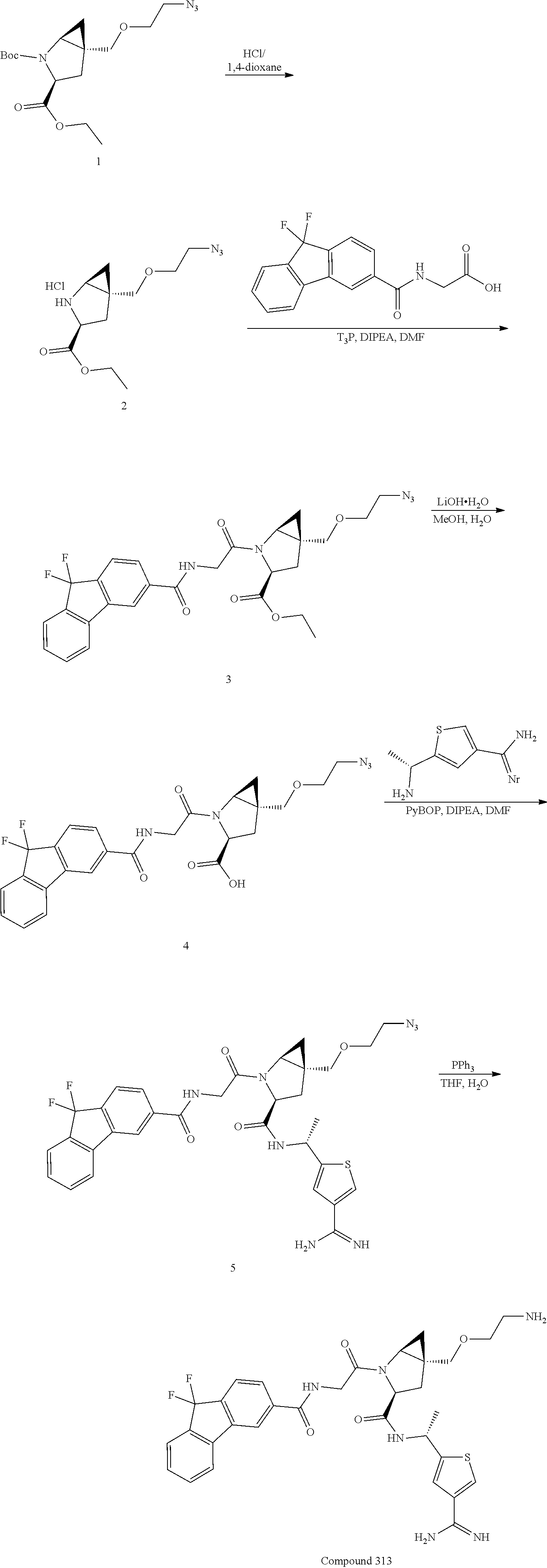

### Step 1: ethyl (1S,3S,5R)-5-((2-azidoethoxy)methyl)-2-azabicyclo[3.1.0]hexane-3-carboxylate hydrochloride (2)

To a solution of 2-(tert-butyl) 3-ethyl (1S,3S,5R)-5-((2-azidoethoxy)methyl)-2-azabicyclo[3.1.0]hexane-2,3-dicarboxylate (300 mg, 0.85 mmol) in HCl/1,4-dioxane (3 mL, 4M) was stirred at room temperature for 1 hour. The mixture was concentrated to dryness under reduced pressure to give ethyl (1 S,3S,5R)-5-((2-azidoethoxy)methyl)-2-azabicyclo[3.1.0]hexane-3-carboxylate hydrochloride (215 mg, yield 99.9%) as a yellow oil, which was directly used in the next reaction without purification. LC/MS (ESI) m/z: 255 (M+H)⁺.

### Step 2: ethyl (1S,3S,5R)-5-((2-azidoethoxy)methyl)-2-((9,9-difluoro-9H-fluorene-3-carbonyl)glycyl)-2-azabicyclo[3.1.0]hexane-3-carboxylate (3)

To a mixture of ethyl (1S,3S,5R)-5-((2-azidoethoxy)methyl)-2-azabicyclo[3.1.0]hexane-3-carboxylate hydrochloride (215 mg, 0.85 mmol) and (9,9-difluoro-9H-fluorene-3-carbonyl)glycine (213.7 mg, 0.71 mmol) in DMF (3 mL) was added DIPEA (545.9 mg, 4.24 mmol) and T₃P (1.35 g, 4.24 mmol, 50% wt. in EtOAc) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give ethyl (1 S,3S,5R)-5-((2-azidoethoxy)methyl)-2-((9,9-difluoro-9H-fluorene-3-carbonyl)glycyl)-2-azabicyclo[3.1.0]hexane-3-carboxylate (290 mg, yield 76.3%) as a yellow oil. LCMS (ESI) m/z: 540 (M+H)⁺.

### Step 3: (1S,3S,5R)-5-((2-azidoethoxy)methyl)-2-((9,9-difluoro-9H-fluorene-3-carbonyl)glycyl)-2-azabicyclo[3.1.0]hexane-3-carboxylic acid (4)

To a solution of ethyl (1S,3S,5R)-5-((2-azidoethoxy)methyl)-2-((9,9-difluoro-9H-fluorene-3-carbonyl)glycyl)-2-azabicyclo[3.1.0]hexane-3-carboxylate (290 mg, 0.54 mmol) in MeOH (3.0 mL) and water (1.0 mL) was added LiOH·H₂O (68 mg, 1.62 mmol) at 0° C. and the mixture was stirred at room temperature for 1 hour. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (1 S,3S,5R)-5-((2-azidoethoxy)methyl)-2-((9,9-difluoro-9H-fluorene-3-carbonyl)glycyl)-2-azabicyclo [3.1.0]hexane-3-carboxylic acid (260 mg, yield 94.6%) as a yellow oil, which was used directly in the next reaction. LCMS (ESI) (m/z): 512 (M+H)⁺.

### Step 4: (1S,3S,5R)-5-((2-azidoethoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (5)

To a mixture of (1 S,3S,5R)-5-((2-azidoethoxy)methyl)-2-((9,9-difluoro-9H-fluorene-3-carbonyl)glycyl)-2-azabicyclo[3.1.0]hexane-3-carboxylic acid (260 mg, 0.51 mmol) and (R)-5-(1-aminoethyl)thiophene-3-carboximidamide (172 mg, 1.02 mmol) in DMF (3 mL) was added DIPEA (393.8 mg, 3.06 mmol) and PyBOP (317.5 mg, 0.61 mmol) at 0° C. and the mixture was stirred under N₂ atmosphere at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) to give (1S,3S,5R)-5-((2-azidoethoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (150 mg, yield 44.5%) as a white solid. LCMS (ESI) (m/z): 663 (M+H)⁺.

### Step 5: (1S,3S,5R)-5-((2-aminoethoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 313)

To a solution of (1 S,3S,5R)-5-((2-azidoethoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (50 mg, 0.076 mmol) in THE (5.0 mL) and water (0.5 mL) was added PPh₃ (39.8 mg, 0.15 mmol) at 0° C. and the mixture was stirred under N₂ atmosphere at room temperature for 16 hours. The mixture was concentrated under reduced pressure to dryness. The residue was purified by prep-TLC (DCM:MeOH=10:1) and further purified by prep-HPLC to give (1S,3S,5R)-5-((2-aminoethoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (3.0 mg, yield 6.25%) as a white solid. ¹H NMR (400 MHz, CD₃OD) δ 8.52 (s, 2H), 8.23-8.18 (m, 2H), 7.88 (dd, J=7.9, 1.4 Hz, 1H), 7.76 (d, J=7.3 Hz, 2H), 7.67 (d, J=7.7 Hz, 1H), 7.59 (t, J=7.7 Hz, 1H), 7.51 (s, 1H), 7.46 (t, J=7.6 Hz, 1H), 5.24 (d, J=7.7 Hz, 1H), 4.53 (d, J=16.6 Hz, 1H), 4.27 (d, J=16.4 Hz, 1H), 3.72-3.68 (m, 2H), 3.66 (d, J=3.1 Hz, 2H), 3.59-3.48 (m, 2H), 3.16-3.13 (m, 2H), 2.73 (t, J=12.7 Hz, 1H), 2.16 (dd, J=13.4, 3.6 Hz, 1H), 1.58 (d, J=7.0 Hz, 3H), 1.42 (s, 1H), 1.07 (t, J=6.4 Hz, 1H). LCMS (ESI) (m/z): 637 (M+H)⁺.

### Scheme 304: Synthesis of (S)—N—((R)-1-(4-carbamoylthiophen-2-yl)ethyl)-7-((9,9-difluoro-9H-fluorene-3-carbonyl)glycyl)-1,4-dioxa-7-azaspiro[4.4]nonane-8-carboxamide (Compound 314)

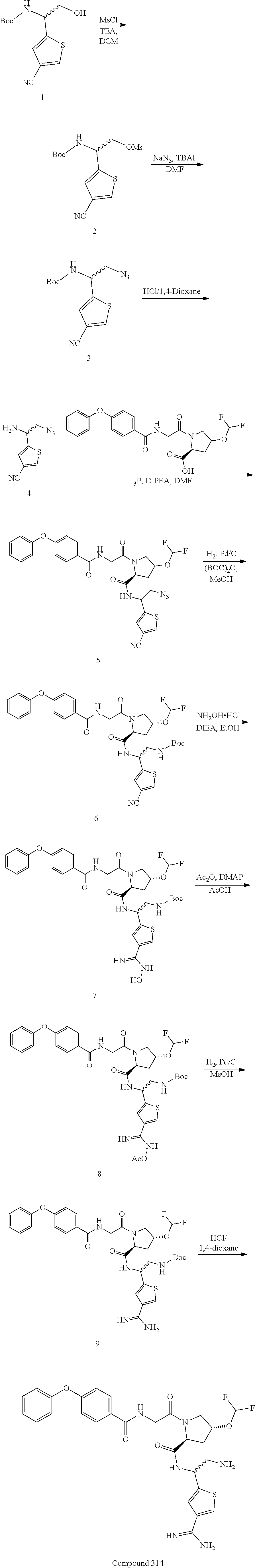

### Step 1: 2-((tert-butoxycarbonyl)amino)-2-(4-cyanothiophen-2-yl)ethyl methanesulfonate

To a mixture of (9,9-difluoro-9H-fluorene-2-carbonyl)glycine (300 mg, 1.1 mmol) in DMF (3 mL) was added TEA (1 g, 10 mmol) and methanesulfonyl chloride (0.384 g, 3.4 mmol) at 0° C. under N₂ atmosphere, and the mixture was stirred at 25° C. for 4 hours. The reaction mixture was diluted with water and extracted with DCM twice. The combined organic layers were washed with brine, dried over anhydrous Na₂SO₄, filtered, and concentrated to dryness under reduced pressure to give 2-((tert-butoxycarbonyl)amino)-2-(4-cyanothiophen-2-yl)ethyl methanesulfonate (386 mg, yield 99.7%) as a white solid. LC/MS (ESI) (m/z): 347 (M+H)⁺.

### Step 2: tert-butyl (2-azido-1-(4-cyanothiophen-2-yl)ethyl)carbamate

To a mixture of 2-((tert-butoxycarbonyl)amino)-2-(4-cyanothiophen-2-yl)ethyl methanesulfonate (385 mg, 1.1 mmol) in DMF (5 ml) was added sodium azide (289 mg, 4.4 mmol) and TBAI (205 mg,0.56 mmol) under N₂ atmosphere and the mixture was stirred at room temperature for 2 days. The reaction mixture was diluted with water and extracted with EtOAc twice. The combined organic layers were washed with brine, dried over anhydrous Na₂SO₄, filtered, and concentrated to dryness under reduced pressure. The residue was purified by flash chromatography (silica gel, 0-50% EtOAc in PE) to give tert-butyl (2-azido-1-(4-cyanothiophen-2-yl)ethyl)carbamate (231 mg, yield 70.9%) as a white solid. LC/MS (ESI) m/z: 294 (M+H)⁺.

### Step 3: 5-(1-amino-2-azidoethyl)thiophene-3-carbonitrile

To a solution of tert-butyl (2-azido-1-(4-cyanothiophen-2-yl)ethyl)carbamate (99 mg, 0.4 mmol) in DCM (1 mL) was added HCl/1,4-dioxane (2 mL, 4M) and the mixture was stirred at room temperature for 1 hour. The reaction mixture was concentrated under reduced pressure to dryness to give 5-(1-amino-2-azidoethyl)thiophene-3-carbonitrile (61 mg, crude) as a white solid, which was used directly in the next step without further purification. LC/MS (ESI) m/z: 194 (M+H)⁺.

### Step 4: (2S,4R)—N-(2-azido-1-(4-cyanothiophen-2-yl)ethyl)-4-(difluoromethoxy)-1-((4-phenoxybenzoyl)glycyl)pyrrolidine-2-carboxamide

To a mixture of 5-(1-amino-2-azidoethyl)thiophene-3-carbonitrile (120 mg, 0.44 mmol) and (2S,4R)-4-(difluoromethoxy)-1-((4-phenoxybenzoyl)glycyl)pyrrolidine-2-carboxylic acid (86 mg, 0.44 mmol) in DMF (3 mL) was added DIPEA (229 mg, 1.8 mmol) and T₃P (211 mg, 0.66 mmol, 50% wt. in EtOAc) under N₂ atmosphere and the mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-40% EtOAc in PE) to give (2S,4R)—N-(2-azido-1-(4-cyanothiophen-2-yl)ethyl)-4-(difluoromethoxy)-1-((4-phenoxybenzoyl) glycyl)pyrrolidine-2-carboxamide (176 mg, yield 88.7%) as a yellow oil. LC/MS (ESI) m/z: 610 (M+H)⁺.

### Step 5: tert-butyl (2-(4-cyanothiophen-2-yl)-2-((2S,4R)-4-(difluoromethoxy)-1-((4-phenoxybenzoyl)glycyl)pyrrolidine-2-carboxamido)ethyl)carbamate

To a solution of (2S,4R)—N-(2-azido-1-(4-cyanothiophen-2-yl)ethyl)-4-(difluoromethoxy)-1-((4-phenoxybenzoyl)glycyl)pyrrolidine-2-carboxamide (40 mg, 0.07 mmol) in MeOH (2 mL) was added Pd/C (15 mg, 10% wt.) and (Boc)₂O (57 mg, 0.26 mmol) at room temperature and the mixture was degassed under N₂ atmosphere for three times and stirred under a H₂ balloon at 25° C. for 2 hours. The mixture was filtered, and the filtrate was concentrated to dryness under reduced pressure to give tert-butyl (2-(4-cyanothiophen-2-yl)-2-((2S,4R)-4-(difluoromethoxy)-1-((4-phenoxybenzoyl) glycyl)pyrrolidine-2-carboxamido)ethyl)carbamate (42 mg, yield 93.6%) as a white solid, which was used directly in the next step. LCMS (ESI) (m/z): 684 (M+H)⁺.

### Step 6: tert-butyl (2-((2S,4R)-4-(difluoromethoxy)-1-((4-phenoxybenzoyl)glycyl) pyrrolidine-2-carboxamido)-2-(4-(N-hydroxycarbamimidoyl)thiophen-2-yl)ethyl) carbamate

To a solution of tert-butyl (2-(4-cyanothiophen-2-yl)-2-((2S,4R)-4-(difluoromethoxy)-1-((4-phenoxybenzoyl)glycyl)pyrrolidine-2-carboxamido)ethyl)carbamate (41 mg, 0.06 mmol) in EtOH (3 mL) was added NH₂OH·HCl (10 mg, 0.15 mmol) and DIPEA (39 mg, 0.3 mmol) at 0° C., and the mixture was stirred at room temperature for 2 hours. The mixture was diluted with water and extracted with EtOAc twice. The combined organic layers were washed with brine, dried over anhydrous Na₂SO₄, filtered, and concentrated under reduced pressure to dryness. The residue was purified by flash column chromatography (silica gel, 0-10% MeOH in DCM) to give tert-butyl (2-((2S,4R)-4-(difluoromethoxy)-1-((4-phenoxybenzoyl)glycyl)pyrrolidine-2-carboxamido)-2-(4-(N-hydroxycarbamimidoyl)thiophen-2-yl)ethyl)carbamate (180 mg, yield 97.7%) as a white solid. LCMS (ESI) m/z: 717 (M+H)⁺.

### Step 7: tert-butyl (2-(4-(N-acetoxycarbamimidoyl)thiophen-2-yl)-2-((2S,4R)-4-(difluoromethoxy)-1-((4-phenoxybenzoyl)glycyl)pyrrolidine-2-carboxamido)ethyl) carbamate

To a solution of tert-butyl (2-((2S,4R)-4-(difluoromethoxy)-1-((4-phenoxybenzoyl)glycyl)pyrrolidine-2-carboxamido)-2-(4-(N-hydroxycarbamimidoyl) thiophen-2-yl)ethyl)carbamate (42 mg, 0.06 mmol) in AcOH (1 mL) was added Ac₂O (1 mL) and DMAP (4 mg, 0.03 mmol) at 0° C. under N₂ atmosphere and the mixture was stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash column chromatography (silica gel, 0-10% MeOH in DCM) to give tert-butyl (2-(4-(N-acetoxycarbamimidoyl)thiophen-2-yl)-2-((2S,4R)-4-(difluoromethoxy)-1-((4-phenoxybenzoyl)glycyl)pyrrolidine-2-carboxamido) ethyl)carbamate (44 mg, yield 97.6%) as a yellow oil. LCMS (ESI) m/z: 759 (M+H)⁺.

### Step 8: tert-butyl (2-(4-carbamimidoylthiophen-2-yl)-2-((2S,4R)-4-(difluoromethoxy)-1-((4-phenoxybenzoyl)glycyl)pyrrolidine-2-carboxamido)ethyl)carbamate

To a solution of tert-butyl (2-(4-(N-acetoxycarbamimidoyl)thiophen-2-yl)-2-((2S,4R)-4-(difluoromethoxy)-1-((4-phenoxybenzoyl)glycyl)pyrrolidine-2-carboxamido)ethyl) carbamate (20 mg, 0.03 mmol) in MeOH (2 mL) was added Pd/C (10 mg, 10% wt.) at room temperature and the mixture was degassed under N₂ atmosphere for three times and stirred under a H₂ balloon at room temperature for 6 hours. The mixture was filtered, and the filtrate was concentrated to dryness under reduced pressure to give tert-butyl (2-(4-carbamimidoylthiophen-2-yl)-2-((2S,4R)-4-(difluoromethoxy)-1-((4-phenoxybenzoyl)glycyl)pyrrolidine-2-carboxamido)ethyl)carbamate (17 mg, yield 92%) as a yellow oil, which was used directly in the next step. LCMS (ESI) (m/z): 701 (M+H)⁺.

### Step 9: (2S,4R)—N-(2-amino-1-(4-carbamimidoylthiophen-2-yl)ethyl)-4-(difluoromethoxy)-1-((4-phenoxybenzoyl)glycyl)pyrrolidine-2-carboxamide (Compound 315)

A solution of tert-butyl (2-(4-carbamimidoylthiophen-2-yl)-2-((2S,4R)-4-(difluoromethoxy)-1-((4-phenoxybenzoyl)glycyl)pyrrolidine-2-carboxamido)ethyl) carbamate (20 mg, 0.03 mmol) in HCl/1,4-dioxane (2 mL, 4M) was stirred under N₂ atmosphere at room temperature for 1 hour. The reaction mixture was concentrated to dryness under reduced pressure and further purified by prep-HPLC to give (2S,4R)-4-(difluoromethoxy)pyrrolidine-2-carboxylate hydrochloride (2.5 mg, yield 14.6%) as a white solid. ¹H NMR (400 MHz, CD₃OD) δ 8.50 (s, 2H), 8.35-8.20 (m, 1H), 7.86 (dd, J=8.6, 4.9 Hz, 2H), 7.58 (d, J=24.3 Hz, 1H), 7.42 (t, J=7.9 Hz, 2H), 7.21 (t, J=7.4 Hz, 1H), 7.06 (d, J=7.8 Hz, 2H), 7.01 (d, J=8.6 Hz, 2H), 6.51 (dd, J=85.1, 63.8 Hz, 1H), 5.45 (ddd, J=9.7, 8.8, 4.2 Hz, 1H), 5.05-5.02 (m, 1H), 4.59-4.53 (m, 1H), 4.30-4.08 (m, 2H), 4.05-3.95 (m, 1H), 3.93-3.84 (m, 1H), 3.69 (ddd, J=11.8, 9.7, 4.8 Hz, 1H), 3.42-3.35 (m, 1H), 2.60-2.41 (m, 1H), 2.31-2.15 (m, 1H).LC/MS (ESI) m/z: 601 (M+H)⁺.

### Scheme 305: Synthesis of (1S,3S,5S)-5-(((2-acetamidoethyl)amino)methyl)-N—((R)-1-(4-carbamimidoylthiophen-2-yl)ethyl)-2-((9,9-difluoro-9H-fluorene-3-carbonyl) glycyl)-2-azabicyclo[3.1.0]hexane-3-carboxamide (Compound 315)

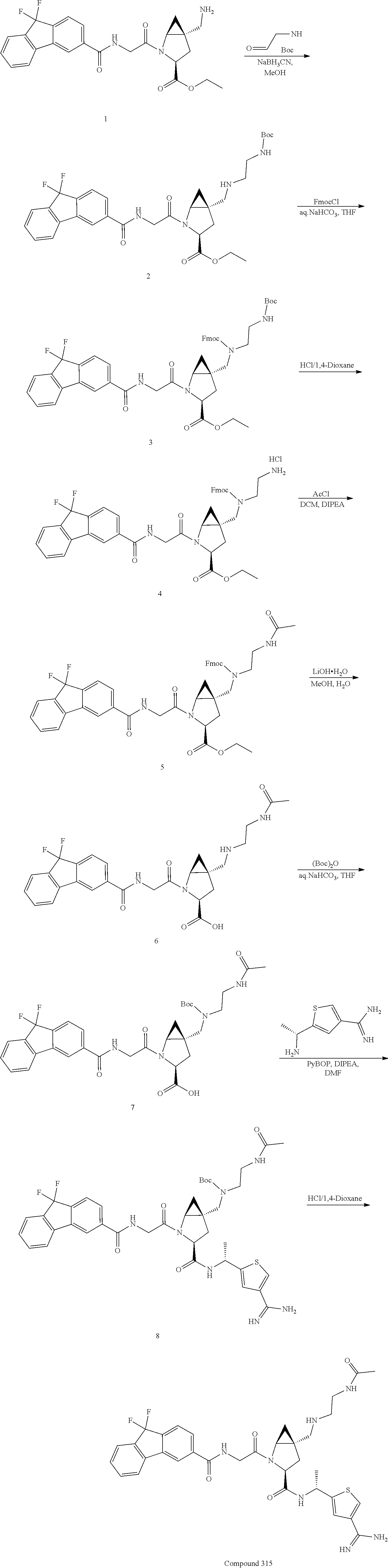

Step 1: ethyl (1S,3S,5S)-5-(((2-((tert-butoxycarbonyl)amino)ethyl)amino)methyl)-2-((9,9-difluoro-9H-fluorene-3-carbonyl)glycyl)-2-azabicyclo[3.1.0]hexane-3-carboxylate

To a mixture of ethyl (1S,3S,5S)-5-(aminomethyl)-2-((9,9-difluoro-9H-fluorene-3-carbonyl)glycyl)-2-azabicyclo[3.1.0]hexane-3-carboxylate (100 mg, 0.21 mmol) and tert-butyl (2-oxoethyl)carbamate (34 mg, 0.21 mmol) in MeOH (3 mL) was added NaBH₃CN (67 mg, 1.05 mmol) and the reaction mixture was stirred at room temperature for 16 hours. The mixture was concentrated under reduced pressure to dryness. The residue was purified by flash chromatography (silica gel, 0-7% MeOH in DCM) to give ethyl (1 S,3S,5S)-5-(((2-((tert-butoxycarbonyl)amino)ethyl)amino) methyl)-2-((9,9-difluoro-9H-fluorene-3-carbonyl)glycyl)-2-azabicyclo[3.1.0]hexane-3-carboxylate (107 mg, yield 82.3%) as a white solid. LCMS (ESI) m/z: 613 (M+H)⁺.

### Step 2: ethyl (1S,3S,5S)-5-(((((9H-fluoren-9-yl)methoxy)carbonyl)(2-((tert-butoxycarbonyl)amino)ethyl)amino)methyl)-2-((9,9-difluoro-9H-fluorene-3-carbonyl)glycyl)-2-azabicyclo[3.1.0]hexane-3-carboxylate

To a mixture of ethyl (1S,3S,5S)-5-(((2-((tert-butoxycarbonyl)amino)ethyl) amino)methyl)-2-((9,9-difluoro-9H-fluorene-3-carbonyl)glycyl)-2-azabicyclo[3.1.0]hexane-3-carboxylate (106 mg, 0.17 mmol) and NaHCO₃ (69 mg, 0.85 mmol) in THE (2 mL) and water (2 mL) was added FmocCl (51 mg, 0.20 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give ethyl (1 S,3S,5S)-5-(((((9H-fluoren-9-yl)methoxy)carbonyl)(2-((tert-butoxycarbonyl)amino) ethyl)amino)methyl)-2-((9,9-difluoro-9H-fluorene-3-carbonyl)glycyl)-2-azabicyclo [3.1.0]hexane-3-carboxylate (100 mg, yield 69.4%) as a white solid. LCMS (ESI) m/z: 835 (M+H)⁺.

### Step 3: ethyl (1S,3S,5S)-5-(((((9H-fluoren-9-yl)methoxy)carbonyl)(2-aminoethyl) amino)methyl)-2-((9,9-difluoro-9H-fluorene-3-carbonyl)glycyl)-2-azabicyclo[3.1.0]hexane-3-carboxylate hydrochloride

A solution of ethyl (1 S,3S,5S)-5-(((((9H-fluoren-9-yl)methoxy)carbonyl)(2-((tert-butoxycarbonyl)amino)ethyl)amino)methyl)-2-((9,9-difluoro-9H-fluorene-3-carbonyl) glycyl)-2-azabicyclo[3.1.0]hexane-3-carboxylate (100 mg, 0.12 mmol) in HCl/1,4-dioxane (3 mL, 4M) was stirred at room temperature for 1 hour. The mixture was concentrated to dryness under reduced pressure to give ethyl (1 S,3S,5S)-5-(((((9H-fluoren-9-yl)methoxy)carbonyl)(2-aminoethyl)amino)methyl)-2-((9,9-difluoro-9H-fluorene-3-carbonyl)glycyl)-2-azabicyclo[3.1.0]hexane-3-carboxylate hydrochloride (90 mg, yield 97.8%) as a white solid, which was directly used in the next reaction without further purification. LC/MS (ESI) m/z: 735 (M+H)⁺.

### Step 4: ethyl (1S,3S,5S)-5-(((((9H-fluoren-9-yl)methoxy)carbonyl)(2-acetamidoethyl) amino)methyl)-2-((9,9-difluoro-9H-fluorene-3-carbonyl)glycyl)-2-azabicyclo[3.1.0]hexane-3-carboxylate

To a mixture of ethyl (1S,3S,5S)-5-(((((9H-fluoren-9-yl)methoxy)carbonyl)(2-aminoethyl)amino)methyl)-2-((9,9-difluoro-9H-fluorene-3-carbonyl)glycyl)-2-azabicyclo[3.1.0]hexane-3-carboxylate hydrochloride (100 mg, 0.14 mmol) and DIPEA (88 mg, 0.70 mmol) in DCM (3 mL) was added AcCl (16 mg, 0.21 mmol) at 0° C. and the mixture was stirred under N₂ atmosphere at room temperature for 1 hour. The mixture was diluted with EtOAc, washed with water and brine, dried over anhydrous Na₂SO₄, filtered, and concentrated under reduced pressure to give ethyl (1S,3S,5S)-5-(((((9H-fluoren-9-yl)methoxy)carbonyl)(2-acetamidoethyl)amino)methyl)-2-((9,9-difluoro-9H-fluorene-3-carbonyl)glycyl)-2-azabicyclo[3.1.0]hexane-3-carboxylate (100 mg, yield 94.3%) as a yellow solid. LCMS (ESI) (m/z): 777 (M+H)⁺.

### Step 5: (1S,3S,5S)-5-(((2-acetamidoethyl)amino)methyl)-2-((9,9-difluoro-9H-fluorene-3-carbonyl)glycyl)-2-azabicyclo[3.1.0]hexane-3-carboxylic acid

To a solution of ethyl (1 S,3S,5S)-5-(((((9H-fluoren-9-yl)methoxy)carbonyl)(2-acetamidoethyl)amino)methyl)-2-((9,9-difluoro-9H-fluorene-3-carbonyl)glycyl)-2-azabicyclo[3.1.0]hexane-3-carboxylate (115 mg, 0.15 mmol) in MeOH (3.0 mL) and water (1 mL) was added LiOH·H₂O (12.5 mg, 0.30 mmol) at 0° C. and the mixture was stirred at room temperature for 1 hour. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (1S,3S,5S)-5-(((2-acetamidoethyl)amino)methyl)-2-((9,9-difluoro-9H-fluorene-3-carbonyl)glycyl)-2-azabicyclo[3.1.0]hexane-3-carboxylic acid (75 mg, yield 96.1%) as a yellow solid, which was used directly in the next reaction. LCMS (ESI) (m/z): 527 (M+H)⁺.

### Step 6: (1S,3S,5S)-5-(((2-acetamidoethyl)(tert-butoxycarbonyl)amino)methyl)-2-((9,9-difluoro-9H-fluorene-3-carbonyl)glycyl)-2-azabicyclo[3.1.0]hexane-3-carboxylic acid

To a mixture of (1 S,3S,5S)-5-(((2-acetamidoethyl)amino)methyl)-2-((9,9-difluoro-9H-fluorene-3-carbonyl)glycyl)-2-azabicyclo[3.1.0]hexane-3-carboxylic acid (75 mg, 0.14 mmol) and NaHCO₃ (60 mg, 0.70 mmol) in THF (2 mL) and water (2 mL) was added Boc₂O (46 mg, 0.21 mmol) under N₂ atmosphere and the reaction mixture was stirred at room temperature for 2 hours. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (1S,3S,5S)-5-(((2-acetamidoethyl)(tert-butoxycarbonyl)amino) methyl)-2-((9,9-difluoro-9H-fluorene-3-carbonyl)glycyl)-2-azabicyclo[3.1.0]hexane-3-carboxylic acid (60 mg, yield 67.4%) as a white solid, which was used directly in the next reaction. LCMS (ESI) (m/z): 627 (M+H)⁺.

### Step 7: tert-butyl (2-acetamidoethyl)(((1S,3S,5S)-3-(((R)-1-(4-carbamimidoylthiophen-2-yl)ethyl)carbamoyl)-2-((9,9-difluoro-9H-fluorene-3-carbonyl)glycyl)-2-azabicyclo[3.1.0]hexan-5-yl)methyl)carbamate

To a mixture of (1 S,3S,5S)-5-(((2-acetamidoethyl)(tert-butoxycarbonyl)amino) methyl)-2-((9,9-difluoro-9H-fluorene-3-carbonyl)glycyl)-2-azabicyclo[3.1.0]hexane-3-carboxylic acid (60 mg, 0.10 mmol) and (R)-5-(1-aminoethyl)thiophene-3-carboximidamide (32 mg, 0.20 mmol) in DMF (2.0 mL) was added DIPEA (78 mg, 0.60 mmol) and PyBOP (78 mg, 0.15 mmol) at 0° C. and the mixture was stirred under N₂ atmosphere at room temperature for 2 hours. The mixture was concentrated under reduced pressure to dryness. The residue was purified by prep-TLC (DCM:MeOH=10:1) to give tert-butyl (2-acetamidoethyl)(((1S,3S,5S)-3-(((R)-1-(4-carbamimidoylthiophen-2-yl)ethyl)carbamoyl)-2-((9,9-difluoro-9H-fluorene-3-carbonyl)glycyl)-2-azabicyclo[3.1.0]hexan-5-yl)methyl)carbamate (65 mg, yield 87.8%) as a white solid. LCMS (ESI) (m/z): 778 (M+H)⁺.

### Step 8: (1S,3S,5S)-5-(((2-acetamidoethyl)amino)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 315)

A solution of tert-butyl (2-acetamidoethyl)(((1 S,3S,5S)-3-(((R)-1-(4-carbamimidoylthiophen-2-yl)ethyl)carbamoyl)-2-((9,9-difluoro-9H-fluorene-3-carbonyl)glycyl)-2-azabicyclo[3.1.0]hexan-5-yl)methyl)carbamate (65 mg, 0.08 mmol) in HCl/1,4-dioxane (3 mL, 4M) was stirred at room temperature for 1 hour. The mixture was concentrated to dryness under reduced pressure. The residue was purified by prep-HPLC to give Compound 315 (15.0 mg, yield 26.3%) as a white solid. ¹H NMR (400 MHz, CD₃OD) δ 8.21-8.00 (m, 2H), 7.87 (d, J=7.9 Hz, 1H), 7.73 (dd, J=14.0, 7.8 Hz, 2H), 7.65 (d, J=7.3 Hz, 1H), 7.58 (t, J=7.5 Hz, 1H), 7.50-7.42 (m, 2H), 5.23 (q, J=6.9 Hz, 1H), 4.89 (s, 1H), 4.48 (d, J=16.6 Hz, 1H), 4.31 (d, J=16.5 Hz, 1H), 3.56 (d, J=3.8 Hz, 1H), 3.33 (s, 1H), 3.27 (s, 1H), 2.90-2.61 (m, 5H), 2.16 (dd, J=13.5, 3.7 Hz, 1H), 1.96 (s, 3H), 1.58 (d, J=6.9 Hz, 3H), 1.32 (d, J=3.1 Hz, 1H), 0.97 (t, J=5.7 Hz, 1H). LCMS (ESI) (m/z): 678 (M+H)⁺.

### Scheme 306: Synthesis of (2S,4S)—N—((R)-1-(4-carbamimidoylthiophen-2-yl)ethyl)-1-((9,9-difluoro-9H-fluorene-3-carbonyl)glycyl)-4-(trifluoromethyl)pyrrolidine-2-carboxamide

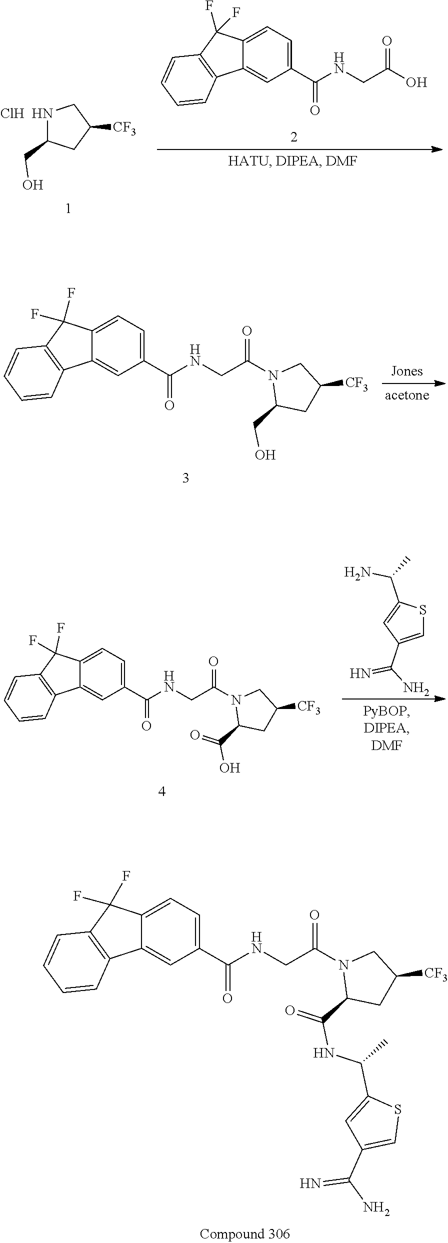

### Step 1: 9,9-difluoro-N-(2-((2S,4S)-2-(hydroxymethyl)-4-(trifluoromethyl)pyrrolidin-1-yl)-2-oxoethyl)-9H-fluorene-3-carboxamide

To a mixture of ((2S,4S)-4-(trifluoromethyl)pyrrolidin-2-yl)methanol hydrochloride (56 mg, 0.33 mmol) and (9,9-difluoro-9H-fluorene-3-carbonyl)glycine (130 mg, 0.43 mmol) in DMF (1 mL) was added HATU (163 mg, 0.43 mmol) and DIPEA (255 mg, 2.0 mmol) under N₂ atmosphere at room temperature and the reaction solution was stirred for 2 hours. The mixture was diluted with EtOAc, washed with water and brine, dried over anhydrous Na₂SO₄, filtered, and concentrated to dryness under reduced pressure. The residue was purified by chromatography (silica gel, 0-50% EtOAc in PE) to give 9,9-difluoro-N-(2-((2S,4S)-2-(hydroxymethyl)-4-(trifluoromethyl)pyrrolidin-1-yl)-2-oxoethyl)-9H-fluorene-3-carboxamide (110 mg, yield 73.4%) as a yellow oil. LCMS (ESI) m/z=455 (M+H)⁺.

### Step 2: (2S,4S)-1-((9,9-difluoro-9H-fluorene-3-carbonyl)glycyl)-4-(trifluoromethyl) pyrrolidine-2-carboxylic acid

To a solution of 9,9-difluoro-N-(2-((2S,4S)-2-(hydroxymethyl)-4-(trifluoromethyl) pyrrolidin-1-yl)-2-oxoethyl)-9H-fluorene-3-carboxamide (110 mg, 0.24 mmol) in acetone (3 mL) was added Jones reagent (12 drops, 2.7 M) under N₂ atmosphere at room temperature and the reaction was stirred for 10 minutes. The mixture was diluted with EtOAc, washed with water and brine, dried over anhydrous Na₂SO₄, filtered, and concentrated to dryness under reduced pressure. The residue was purified by prep-HPLC to give (2S,4S)-1-((9,9-difluoro-9H-fluorene-3-carbonyl)glycyl)-4-(trifluoromethyl)pyrrolidine-2-carboxylic acid (50 mg, yield 45.8%) as a white solid. LCMS (ESI) m/z=469 (M+H)⁺.

### Step 3: Synthesis of (2S,4S)—N—((R)-1-(4-carbamimidoylthiophen-2-yl)ethyl)-1-((9,9-difluoro-9H-fluorene-3-carbonyl)glycyl)-4-(trifluoromethyl)pyrrolidine-2-carboxamide (Compound 322)

To a mixture of (2S,4S)-1-((9,9-difluoro-9H-fluorene-3-carbonyl)glycyl)-4-(trifluoromethyl)pyrrolidine-2-carboxylic acid (20 mg, 0.043 mmol) and (R)-5-(1-aminoethyl)thiophene-3-carboximidamide (15 mg, 0.086 mmol) in DMF (0.5 mL) was added PyBOP (27 mg, 0.052 mmol) and DIPEA (33 mg, 0.26 mmol) under N₂ atmosphere at room temperature and the reaction solution was stirred for 2 hours. The mixture was diluted with EtOAc, washed with water and brine, dried over anhydrous Na₂SO₄, filtered, and concentrated to dryness under reduced pressure. The residue was purified by chromatography (silica gel, 10% MeOH in DCM) and further purified by prep-HPLC to give Compound 322 (7 mg, yield 26.3%) as a white solid. ¹H NMR (400 MHz, CD₃OD) δ 8.52 (s, 1H), 8.26-8.17 (m, 2H), 7.87 (dt, J=8.2, 4.1 Hz, 1H), 7.74 (dd, J=15.7, 7.8 Hz, 2H), 7.65 (d, J=7.8 Hz, 1H), 7.58 (t, J=7.5 Hz, 1H), 7.51 (s, 1H), 7.45 (t, J=7.3 Hz, 1H), 5.28 (q, J=7.0 Hz, 1H), 4.57-4.52 (m, 2H), 4.38 (d, J=16.7 Hz, 1H), 4.18 (dd, J=21.7, 13.4 Hz, 2H), 3.74 (t, J=10.2 Hz, 1H), 2.69-2.58 (m, 1H), 2.07 (dd, J=21.7, 10.8 Hz, 1H), 1.61 (t, J=15.8 Hz, 3H). LCMS (ESI) m/z=620 (M+H)⁺.

### Scheme 307: Synthesis of (2S,4S)—N—((R)-1-(4-carbamimidoylthiophen-2-yl)ethyl)-1-((4-(4-fluorophenoxy)benzoyl)glycyl)-4-(trifluoromethyl)pyrrolidine-2-carboxamide (Compound 323)

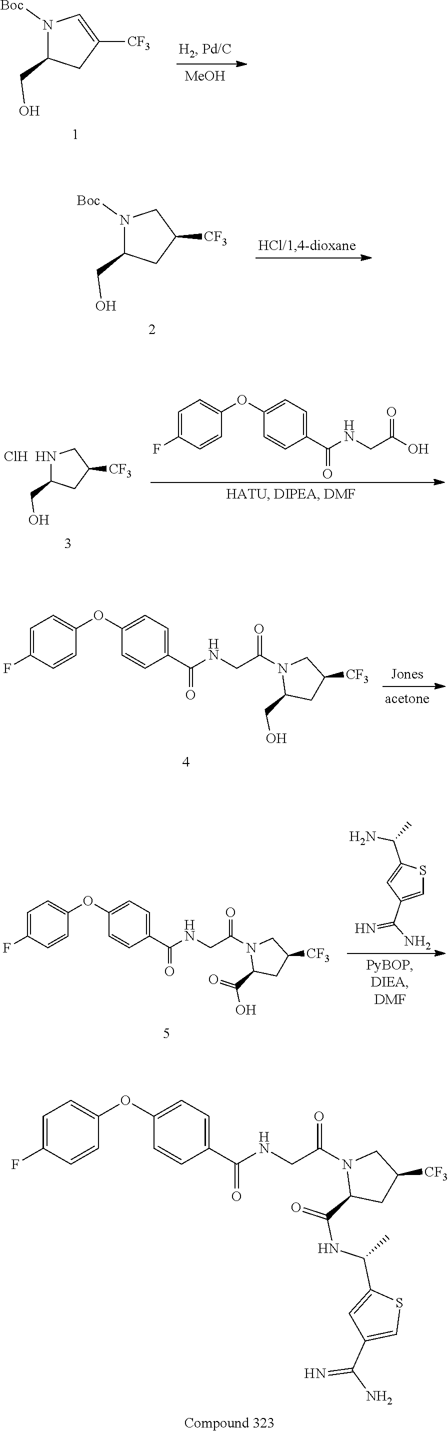

### Step 1: tert-butyl (2S,4S)-2-(hydroxymethyl)-4-(trifluoromethyl)pyrrolidine-1-carboxylate

To a solution of tert-butyl (S)-2-(hydroxymethyl)-4-(trifluoromethyl)-2,3-dihydro-1H-pyrrole-1-carboxylate (250 mg, 0.90 mmol) in MeOH (4 mL) was added Pd/C (60 mg, 10% wt.) at room temperature, the mixture was degassed under N₂ atmosphere for three times and stirred under a H₂ balloon at 25° C. overnight. The mixture was filtered, and the filtrate was concentrated to dryness under reduced pressure to give tert-butyl (2S,4S)-2-(hydroxymethyl)-4-(trifluoromethyl)pyrrolidine-1-carboxylate (200 mg, yield 79.1%) as a yellow oil, which was used directly in the next step. LCMS (ESI) (m/z): 270 (M+H)⁺.

### Step 2: ((2S,4S)-4-(trifluoromethyl)pyrrolidin-2-yl)methanol hydrochloride

To a mixture of tert-butyl (2S,4S)-2-(hydroxymethyl)-4-(trifluoromethyl)pyrrolidine-1-carboxylate (200 mg, 0.74 mmol) in HCl/1,4-dioxane (3 mL) was stirred under N₂ atmosphere at room temperature for 2 hours. The reaction mixture was concentrated to dryness under reduced pressure, co-evaporate with DCM again and dried under vacuum to give ((2S,4S)-4-(trifluoromethyl)pyrrolidin-2-yl)methanol hydrochloride (148 mg, yield 97.3%), which was used directly in the next step without further purification. LCMS (ESI) m/z: 170 (M+H)⁺.

### Step 3: 4-(4-fluorophenoxy)-N-(2-((2S,4S)-2-(hydroxymethyl)-4-(trifluoromethyl) pyrrolidin-1-yl)-2-oxoethyl)benzamide

To a mixture of ((2S,4S)-4-(trifluoromethyl)pyrrolidin-2-yl)methanol hydrochloride (72 mg, 0.35 mmol) and (4-(4-fluorophenoxy)benzoyl)glycine (150 mg, 0.52 mmol) in DMF (1 mL) was added HATU (171 mg, 0.45 mmol) and DIPEA (271 mg, 2.1 mmol) under N₂ atmosphere at room temperature and the reaction was stirred for 2 hours. The mixture was diluted with EtOAc, washed with water and brine, dried over anhydrous Na₂SO₄, filtered, and concentrated to dryness under reduced pressure. The residue was purified by flash chromatography (silica gel, 0-50% EtOAc in PE) and further purified by SFC to give 4-(4-fluorophenoxy)-N-(2-((2S,4S)-2-(hydroxymethyl)-4-(trifluoromethyl)pyrrolidin-1-yl)-2-oxoethyl)benzamide (124 mg, yield 80.5%) as a yellow oil. LCMS (ESI) m/z=441 (M+H)⁺.

### Step 4: (2S,4S)-1-((4-(4-fluorophenoxy)benzoyl)glycyl)-4-(trifluoromethyl) pyrrolidine-2-carboxylic acid

To a solution of 4-(4-fluorophenoxy)-N-(2-((2S,4S)-2-(hydroxymethyl)-4-(trifluoromethyl)pyrrolidin-1-yl)-2-oxoethyl)benzamide (72 mg, 0.16 mmol) in acetone (2 mL) was added Jones reagent (9 drops, 2.7 M) under N₂ atmosphere at room temperature and stirred for 10 minutes. The mixture was diluted with EtOAc, washed with water and brine, dried over anhydrous Na₂SO₄, filtered, and concentrated to dryness under reduced pressure. The residue was purified by prep-HPLC to give (2S,4S)-1-((4-(4-fluorophenoxy)benzoyl)glycyl)-4-(trifluoromethyl)pyrrolidine-2-carboxylic acid (27 mg, yield 37.2%) as a white solid. LCMS (ESI) m/z=455 (M+H)⁺.

### Step 5: (2S,4S)—N—((R)-1-(4-carbamimidoylthiophen-2-yl)ethyl)-1-((4-(4-fluorophenoxy)benzoyl)glycyl)-4-(trifluoromethyl)pyrrolidine-2-carboxamide (Compound 323)

To a mixture of (2S,4S)-1-((4-(4-fluorophenoxy)benzoyl)glycyl)-4-(trifluoromethyl) pyrrolidine-2-carboxylic acid (27 mg, 0.059 mmol) and (R)-5-(1-aminoethyl)thiophene-3-carboximidamide (20 mg, 0.12 mmol) in DMF (0.5 mL) was added PyBOP (37 mg, 0.071 mmol) and DIPEA (46 mg, 0.35 mmol) under N₂ atmosphere at room temperature and stirred for 2 hours. The mixture was diluted with EtOAc, washed with water and brine, dried over anhydrous Na₂SO₄, filtered, and concentrated to dryness under reduced pressure. The residue was purified by chromatography (silica gel, 10% MeOH in DCM) and further purified by prep-HPLC to give (2S,4S)—N—((R)-1-(4-carbamimidoylthiophen-2-yl)ethyl)-1-((4-(4-fluorophenoxy)benzoyl)glycyl)-4-(trifluoromethyl)pyrrolidine-2-carboxamide (8 mg, yield 22.4%) as a white solid. ¹H NMR (400 MHz, CD₃OD) δ 8.22 (t, J=4.0 Hz, 1H), 7.84 (t, J=5.8 Hz, 2H), 7.53 (d, J=9.5 Hz, 1H), 7.18-7.13 (m, 2H), 7.11-7.07 (m, 2H), 6.99 (d, J=8.8 Hz, 2H), 5.27 (q, J=6.8 Hz, 1H), 4.53 (t, J=8.5 Hz, 2H), 4.30 (d, J=16.6 Hz, 1H), 4.13 (dd, J=23.9, 12.4 Hz, 2H), 3.72 (dd, J=21.5, 11.3 Hz, 1H), 2.80-2.58 (m, 1H), 2.05 (d, J=13.5 Hz, 1H), 1.60 (t, J=14.5 Hz, 3H). LCMS (ESI) m/z=606 (M+H)⁺.

### Scheme 308: Synthesis of (2S,4R)—N—((R)-1-(4-carbamimidoylthiophen-2-yl)ethyl)-1-((9,9-difluoro-9H-fluorene-3-carbonyl)glycyl)-4-(trifluoromethyl)pyrrolidine-2-carboxamide (Compound 324)

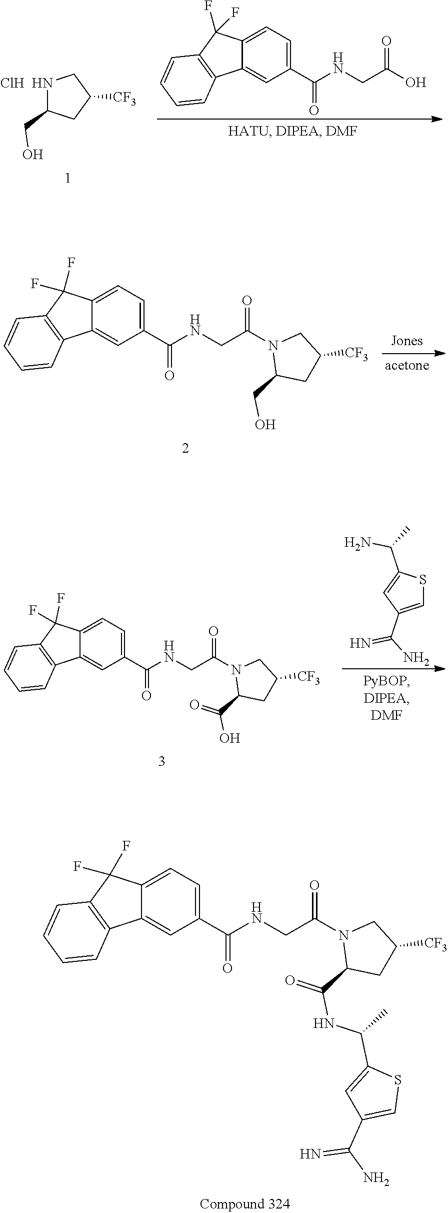

Compound 324 was prepared from ((2S,4R)-4-(trifluoromethyl)pyrrolidin-2-yl)methanol hydrochloride, (9,9-difluoro-9H-fluorene-3-carbonyl)glycine, and (R)-5-(1-aminoethyl)thiophene-3-carboximidamide based on the procedures set forth in Scheme 306. ¹H NMR (400 MHz, CD₃OD) δ 8.53 (s, 1H), 8.21 (dd, J=14.0, 12.4 Hz, 2H), 7.88 (dd, J=7.8, 1.4 Hz, 1H), 7.74 (dd, J=17.6, 7.7 Hz, 2H), 7.65 (d, J=7.5 Hz, 1H), 7.57 (d, J=7.5 Hz, 1H), 7.46 (dd, J=14.1, 6.6 Hz, 2H), 5.29 (dd, J=19.4, 12.9 Hz, 1H), 4.66 (dd, J=8.7, 3.3 Hz, 1H), 4.31-4.05 (m, 3H), 3.88 (dd, J=10.6, 7.6 Hz, 1H), 3.41 (dd, J=16.4, 8.1 Hz, 1H), 2.44 (dd, J=9.0, 4.4 Hz, 1H), 2.29 (ddd, J=13.3, 7.4, 3.5 Hz, 1H), 1.61 (t, J=15.9 Hz, 3H) LCMS (ESI) m/z=620 (M+H)⁺.

### Scheme 309: Synthesis of (S)—N—((R)-1-(4-carbamimidoylthiophen-2-yl)ethyl)-7-((10-methylanthracene-2-carbonyl)glycyl)-1,4-dioxa-7-azaspiro[4.4]nonane-8-carboxamide (Compound 325)

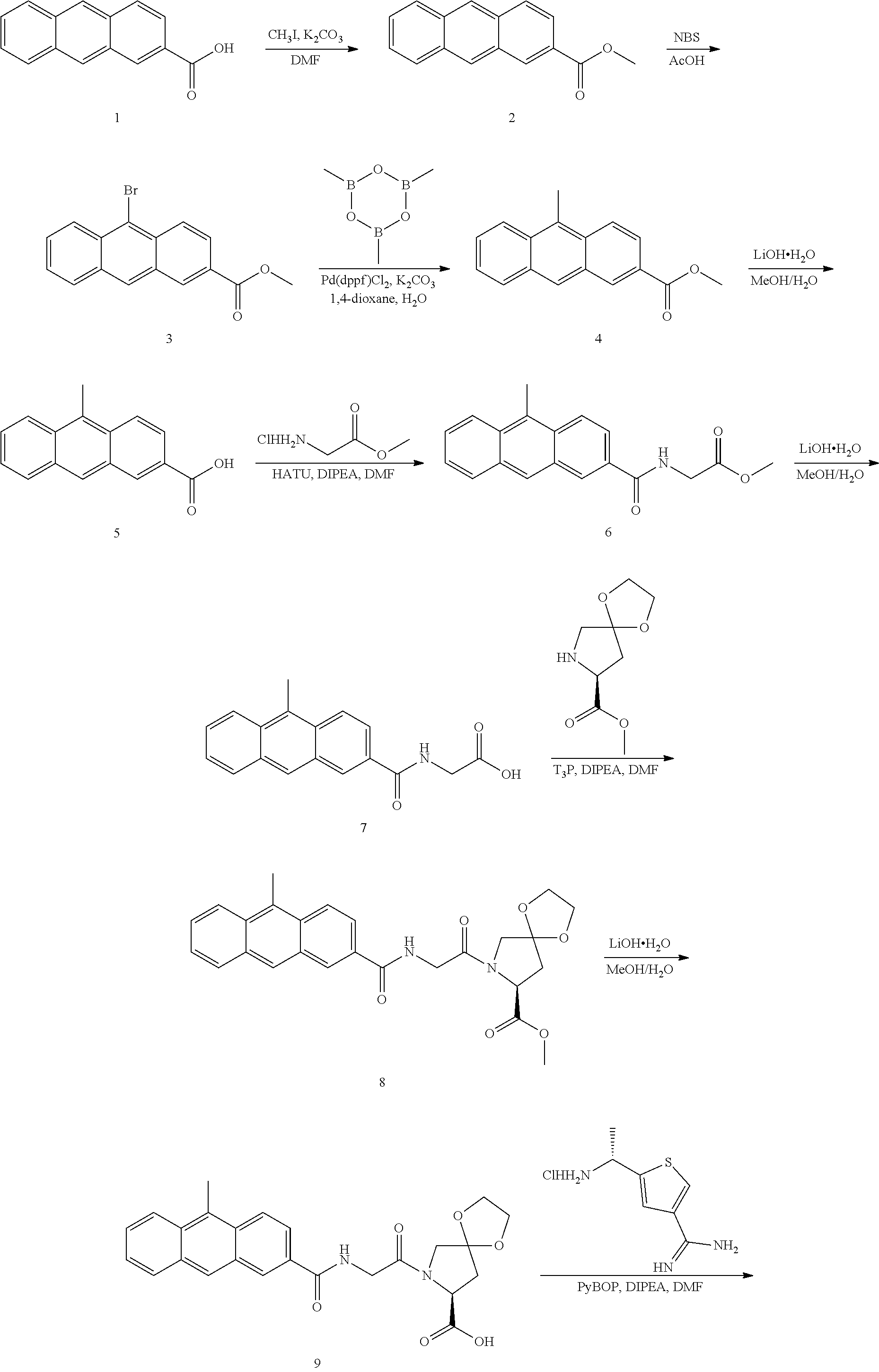

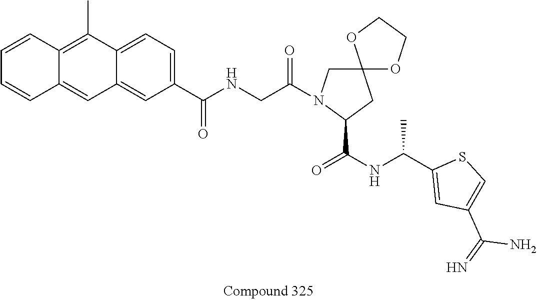 [SMILES: Cc1c2ccccc2cc2cc(C(=O)NCC(=O)N3CC4(C[C@H]3C(=O)N[C@H](C)c3cc(C(=N)N)cs3)OCCO4)ccc12]

### Step 1: methyl anthracene-2-carboxylate

To a mixture of anthracene-2-carboxylic acid (360 mg, 1.62 mmol) and K₂CO₃ (672 mg, 4.86 mmol) in DMF (3 mL) was added iodomethane (1.38 g, 9.72 mmol) and the mixture was stirred at room temperature overnight. The reaction mixture was diluted with EtOAc, washed with brine, dried over anhydrous Na₂SO₄, filtered, and concentrated under reduced pressure to dryness. The residue was purified by flash chromatography (silica gel, 0-30% EtOAc in PE) to give methyl anthracene-2-carboxylate (280 mg, yield 72.9%) as a yellow solid. LC/MS (ESI) m/z: 237 (M+H)⁺.

### Step 2: methyl 10-bromoanthracene-2-carboxylate

A mixture of methyl anthracene-2-carboxylate (280 mg, 1.19 mmol) and NBS (211 mg, 1.19 mmol) in AcOH (3 mL) was stirred at room temperature overnight. The reaction mixture was concentrated under reduced pressure to dryness. The residue was purified by flash chromatography (silica gel, 0-30% EtOAc in PE) and further purified by prep-HPLC to give methyl 10-bromoanthracene-2-carboxylate (90 mg, yield 26.7%) as a yellow solid. LC/MS (ESI) m/z: 315 (M+H)⁺.

### Step 3: methyl 10-methylanthracene-2-carboxylate

To a mixture of methyl 10-bromoanthracene-2-carboxylate (80 mg, 0.25 mmol), Pd(dppf)Cl₂ (21 mg, 0.025 mmol) and K₂CO₃ (105 mg, 0.72 mmol) in 1,4-dioxane (1 mL) and H₂O (0.25 mL) was added 2,4,6-trimethyl-1,3,5,2,4,6-trioxatriborinane (16 mg, 0.13 mmol) under N₂ atmosphere and the mixture was stirred at 80° C. overnight. The reaction mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give methyl 10-methylanthracene-2-carboxylate (50 mg, yield 78.7%) as a yellow solid. ¹H NMR (400 MHz, CDCl₃) δ 8.78 (d, J=1.6 Hz, 1H), 8.46 (s, 1H), 8.33-8.27 (m, 2H), 8.06-7.99 (m, 2H), 7.60-7.54 (m, 1H), 7.53-7.47 (m, 1H), 4.01 (s, 3H), 3.10 (s, 3H).

### Step 4: 10-methylanthracene-2-carboxylic acid

To a solution of methyl 10-methylanthracene-2-carboxylate (46 mg, 0.18 mmol) in MeOH (1 mL) and H₂O (0.3 mL) was added LiOH·H₂O (23 mg, 0.54 mmol) at 0° C. and the mixture was stirred at room temperature for 2 hours. The reaction mixture was acidified with 1 N aq·HCl to pH ˜3 and extracted with EtOAc twice. The combined organic layers were washed with brine, dried over anhydrous Na₂SO₄, filtered, and concentrated to dryness under reduced pressure to give 10-methylanthracene-2-carboxylic acid (36 mg, yield 84.4%) as a yellow solid, which was used in next step without purification. LC/MS (ESI) m/z:237 (M+H)⁺.

### Step 5: methyl (10-methylanthracene-2-carbonyl)glycinate

To a mixture of 10-methylanthracene-2-carboxylic acid (36 mg, 0.15 mmol) and methyl glycinate hydrochloride (23 mg, 0.18 mmol) in DMF (2 mL) was added HATU (87 mg, 0.23 mmol) and DIPEA (59 mg, 0.46 mmol) and the mixture was stirred at room temperature for 2 hours. The reaction mixture was diluted with EtOAc and washed with water and brine, dried over anhydrous Na₂SO₄, filtered, and concentrated under reduced pressure to dryness. The residue was purified by flash chromatography (silica gel, 0-30% EtOAc in PE) to give methyl (10-methylanthracene-2-carbonyl)glycinate (45 mg, yield 97.4%) as a yellow solid. LC/MS (ESI) m/z:308 (M+H)⁺.

### Step 6: (10-methylanthracene-2-carbonyl)glycine

To a solution of methyl (10-methylanthracene-2-carbonyl)glycinate (45 mg, 0.15 mmol) in MeOH (1 mL) and H₂O (0.3 mL) was added LiOH·H₂O (12 mg, 0.29 mmol) at 0° C. and the mixture was stirred at room temperature for 2 hours. The reaction mixture was acidified with 1 N aq·HCl to pH ˜3 and extracted with EtOAc twice. The combined organic layers were washed with brine, dried over anhydrous Na₂SO₄, filtered, and concentrated to dryness under reduced pressure to give (10-methylanthracene-2-carbonyl)glycine (44 mg, yield 99.8%) as a yellow solid, which was used in next step without purification. LC/MS (ESI) m/z:294 (M+H)⁺.

### Step 7: methyl (S)-7-((10-methylanthracene-2-carbonyl)glycyl)-1,4-dioxa-7-azaspiro [4.4]nonane-8-carboxylate

To a mixture of (10-methylanthracene-2-carbonyl)glycine (44 mg, 0.15 mmol) and methyl (S)-1,4-dioxa-7-azaspiro[4.4]nonane-8-carboxylate (42 mg, 0.23 mmol) in DMF (2 mL) was added DIPEA (58 mg, 0.45 mmol) and T₃P (143 mg, 0.23 mmol, 50% wt. in EtOAc) and the mixture was stirred at room temperature for 2 hours. The reaction mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-40% EtOAc in PE) to give methyl (S)-7-((10-methylanthracene-2-carbonyl)glycyl)-1,4-dioxa-7-azaspiro[4.4]nonane-8-carboxylate (59 mg, yield 85.0%) as a yellow solid. LC/MS (ESI) m/z: 463 (M+H)⁺.

### Step 8: (S)-7-((10-methylanthracene-2-carbonyl)glycyl)-1,4-dioxa-7-azaspiro[4.4]nonane-8-carboxylic acid

To a solution of methyl (S)-7-((10-methylanthracene-2-carbonyl)glycyl)-1,4-dioxa-7-azaspiro[4.4]nonane-8-carboxylate (59 mg, 0.13 mmol) in MeOH (1 mL) and H₂O (0.3 mL) was added LiOH·H₂O (11 mg, 0.26 mmol) at 0° C. and the mixture was stirred at room temperature for 2 hours. The reaction mixture was acidified with 1 N aq·HCl to pH ˜3 and extracted with EtOAc twice. The combined organic layers were washed with brine, dried over anhydrous Na₂SO₄, filtered, and concentrated to dryness under reduced pressure to give (S)-7-((10-methylanthracene-2-carbonyl)glycyl)-1,4-dioxa-7-azaspiro[4.4]nonane-8-carboxylic acid (53 mg, yield 91.4%) as a yellow solid, which was used in next step without purification. LC/MS (ESI) m/z:449 (M+H)⁺.

### Step 9: (S)—N—((R)-1-(4-carbamimidoylthiophen-2-yl)ethyl)-7-((10-methylanthracene-2-carbonyl)glycyl)-1,4-dioxa-7-azaspiro[4.4]nonane-8-carboxamide (Compound 325)

To a mixture of (S)-7-((10-methylanthracene-2-carbonyl)glycyl)-1,4-dioxa-7-azaspiro[4.4]nonane-8-carboxylic acid (20 mg, 0.045 mmol) and (R)-5-(1-aminoethyl)thiophene-3-carboximidamide hydrochloride (19 mg, 0.09 mmol) in DMF (2 mL) was added DIPEA (29 mg, 0.23 mmol) and PyBOP (28 mg, 0.054 mmol) at 0° C. and the mixture was stirred at room temperature for 2 hours. The reaction mixture was concentrated under reduced pressure to dryness. The residue was purified by prep-TLC (DCM:MeOH=8:1) and further purified by prep-HPLC to give (S)—N—((R)-1-(4-carbamimidoylthiophen-2-yl)ethyl)-7-((10-methylanthracene-2-carbonyl)glycyl)-1,4-dioxa-7-azaspiro[4.4]nonane-8-carboxamide (2.2 mg, yield 8.1%) as a white solid. ¹H NMR (400 MHz, CD₃OD) δ 8.59 (d, J=1.6 Hz, 1H), 8.50 (d, J=9.0 Hz, 1H), 8.40 (d, J=9.3 Hz, 1H), 8.36 (d, J=8.7 Hz, 1H), 8.07 (t, J=4.8 Hz, 2H), 7.89 (dd, J=9.3, 1.8 Hz, 1H), 7.61-7.57 (m, 1H), 7.55-7.51 (m, 2H), 5.29 (q, J=6.6 Hz, 1H), 4.60 (dd, J=8.8, 6.5 Hz, 1H), 4.22 (dd, J=19.0, 2.3 Hz, 2H), 4.04-3.98 (m, 4H), 3.81 (dd, J=17.2, 6.5 Hz, 2H), 3.11 (s, 3H), 2.45 (dd, J=13.1, 8.9 Hz, 1H), 2.23 (dd, J=13.1, 6.5 Hz, 1H), 1.61 (t, J=19.0 Hz, 3H). LCMS (ESI) (m/z): 600 (M+H)⁺.

### Scheme 310: Synthesis of methyl ((E)-amino(5-((R)-1-((2S,4R)-1-((9,9-difluoro-9H-fluorene-3-carbonyl)glycyl)-4-fluoro-4-(methoxymethyl)pyrrolidine-2-carboxamido) ethyl)thiophen-3-yl)methylene)carbamate (Compound 232)

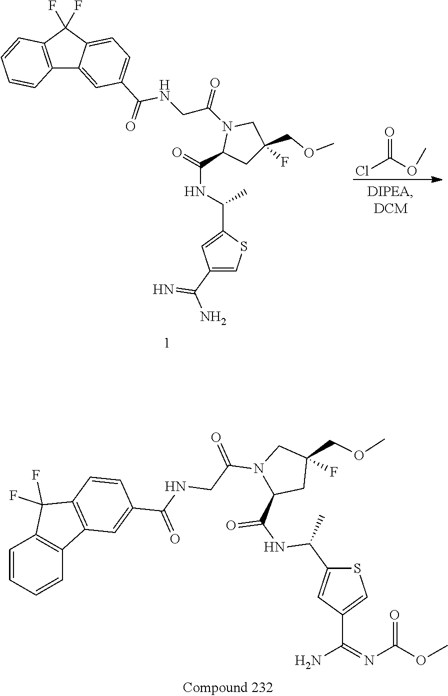

Compound 232 was prepared from (2S,4R)—N—((R)-1-(4-carbamimidoylthiophen-2-yl)ethyl)-1-((9,9-difluoro-9H-fluorene-3-carbonyl)glycyl)-4-fluoro-4-(methoxymethyl)pyrrolidine-2-carboxamide and methyl carbonochloridate based on the procedures set forth in Scheme 143. ¹H NMR (400 MHz, CD₃OD) δ 8.16 (d, J=11.7 Hz, 1H), 8.13-8.02 (m, 1H), 7.87 (t, J=9.9 Hz, 1H), 7.82-7.67 (m, 3H), 7.65 (d, J=7.3 Hz, 1H), 7.58 (dd, J=9.7, 4.8 Hz, 1H), 7.44 (t, J=7.5 Hz, 1H), 5.38-5.18 (m, 1H), 4.74-4.62 (m, 1H), 4.53-4.26 (m, 1H), 4.17-4.10 (m, 1H), 4.07-3.97 (m, 1H), 3.79-3.63 (m, 3H), 3.46 (dd, J=24.9, 21.6 Hz, 6H), 2.81-2.49 (m, 1H), 2.36-2.11 (m, 1H), 1.59 (t, J=17.0 Hz, 3H). LC/MS (ESI) (m/z): 672 (M+H)⁺.

### Scheme 311: Synthesis of N-(2-((2S,4R)-2-(((R)-1-(4-carbamimidoylthiophen-2-yl)ethyl)carbamoyl)-4-(difluoromethoxy)pyrrolidin-1-yl)-2-oxoethyl)-9H-carbazole-3-carboxamide (Compound 326)

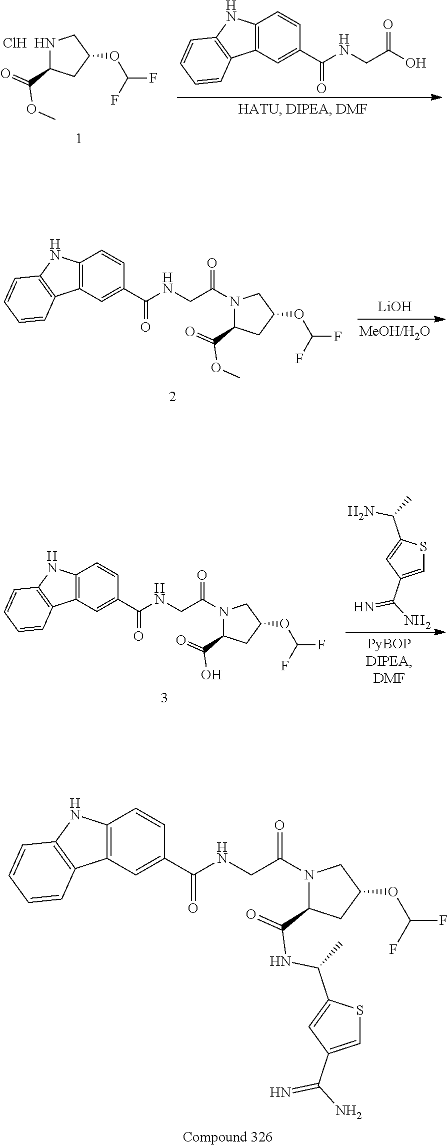

Compound 326 was prepared from methyl (2S,4R)-4-(difluoromethoxy)pyrrolidine-2-carboxylate hydrochloride, (9H-carbazole-3-carbonyl)glycine, and (R)-5-(1-aminoethyl)thiophene-3-carboximidamide based on the procedures set forth in Scheme 3. ¹H NMR (400 MHz, CD₃OD) δ 8.65 (t, J=4.4 Hz, 1H), 8.53 (s, 1H), 8.22 (dd, J=19.8, 1.5 Hz, 1H), 8.10 (d, J=7.9 Hz, 1H), 7.92 (dt, J=12.2, 6.1 Hz, 1H), 7.55 (s, 1H), 7.49 (d, J=8.4 Hz, 2H), 7.42 (d, J=7.2 Hz, 1H), 7.22 (t, J=7.4 Hz, 1H), 6.51 (td, J=74.5, 16.5 Hz, 1H), 5.31-5.24 (m, 1H), 4.64-4.59 (m, 2H), 4.25 (dt, J=29.7, 14.8 Hz, 2H), 4.01-3.90 (m, 2H), 2.54-2.41 (m, 1H), 2.29-2.20 (m, 1H), 1.61 (t, J=15.8 Hz, 3H). LCMS (ESI) m/z=583 (M+H)⁺.

### Scheme 312: Synthesis of (2S,4R)—N—((R)-1-(4-carbamimidoylthiophen-2-yl)ethyl)-4-fluoro-4-(fluoromethyl)-1-((phenoxathiine-3-carbonyl)glycyl)pyrrolidine-2-carboxamide (Compound 327)

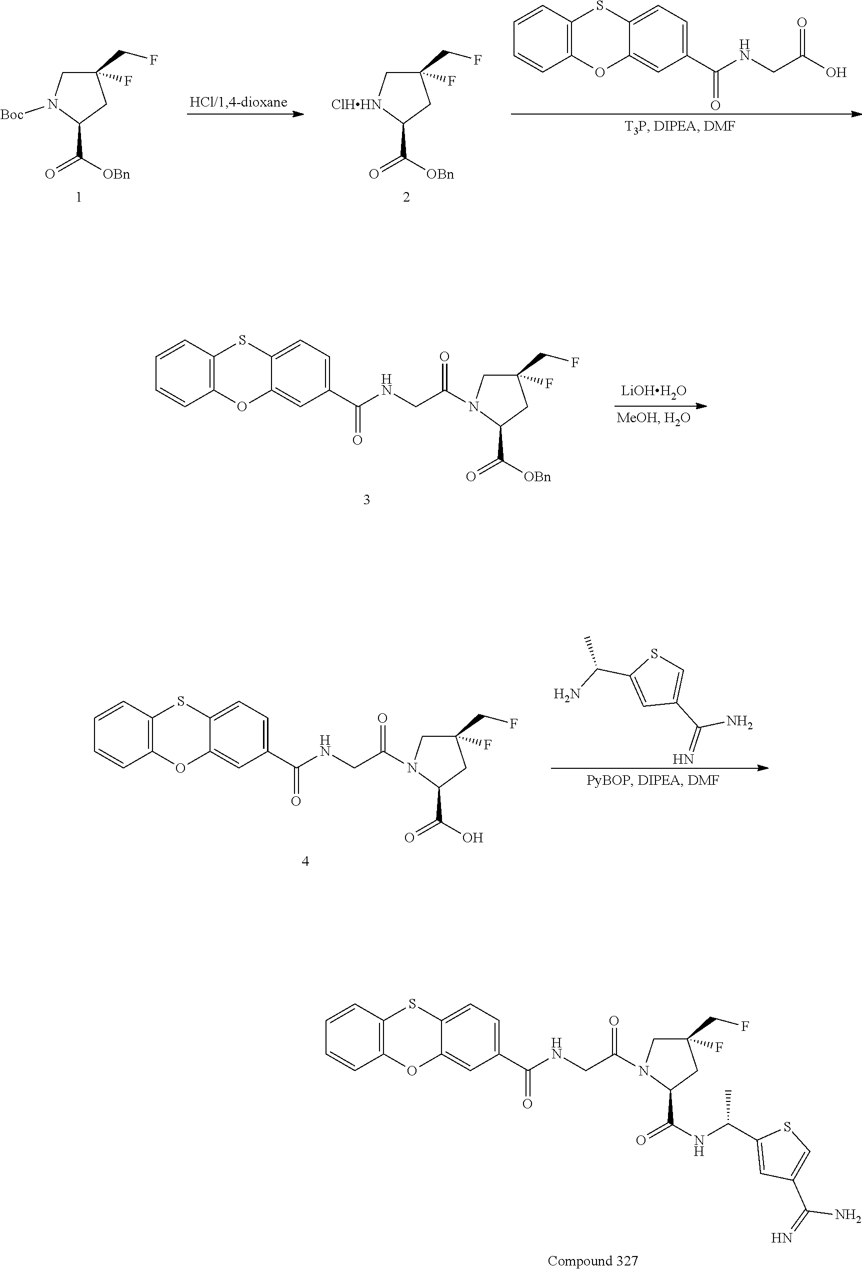

Compound 327 was prepared from 2-benzyl 1-(tert-butyl) (2S,4R)-4-fluoro-4-(fluoromethyl)pyrrolidine-1,2-dicarboxylate, (phenoxathiine-3-carbonyl)glycine, and (R)-5-(1-aminoethyl)thiophene-3-carboximidamide hydrochloride based on the procedures set forth in Scheme 3. ¹H NMR (400 MHz, CD₃OD) δ 8.49 (s, 1H), 8.20 (d, J=1.6 Hz, 1H), 7.51 (ddd, J=16.2, 8.5, 1.6 Hz, 3H), 7.20 (dt, J=6.4, 2.1 Hz, 2H), 7.17-7.13 (m, 1H), 7.11-7.06 (m, 1H), 7.03 (dd, J=8.1, 1.1 Hz, 1H), 5.27 (q, J=7.2 Hz, 1H), 4.81-4.75 (m, 1H), 4.65 (dt, J=13.6, 7.6 Hz, 2H), 4.29 (d, J=16.7 Hz, 1H), 4.12 (t, J=16.3 Hz, 2H), 4.02-3.87 (m, 1H), 2.82-2.56 (m, 1H), 2.22 (ddd, J=35.0, 14.2, 9.2 Hz, 1H), 1.58 (d, J=7.0 Hz, 3H). LCMS (ESI) (m/z): 600 (M+H)⁺.

### Scheme 313: Synthesis of (2S,4R)—N—((R)-1-(4-carbamimidoylthiophen-2-yl)ethyl)-4-(difluoromethoxy)-1-((phenoxathiine-3-carbonyl)glycyl)pyrrolidine-2-carboxamide (Compound 328)

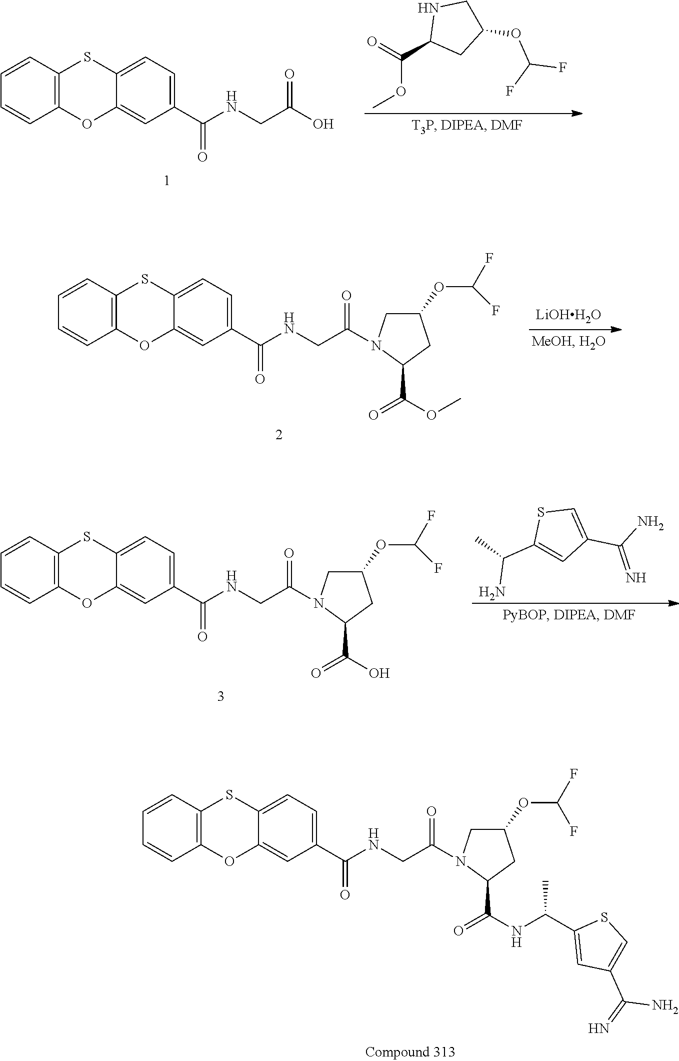

Compound 313 was prepared from (phenoxathiine-3-carbonyl)glycine, methyl (2S,4R)-4-(difluoromethoxy)pyrrolidine-2-carboxylate, and (R)-5-(1-aminoethyl)thiophene-3-carboximidamide based on th procedures set forth in Scheme 3. ¹H NMR (400 MHz, CD₃OD) δ 8.48 (s, 1H), 8.22 (d, J=1.6 Hz, 1H), 7.53 (t, J=1.5 Hz, 2H), 7.50 (d, J=1.8 Hz, 1H), 7.24 (d, J=8.1 Hz, 1H), 7.21-7.18 (m, 1H), 7.15 (s, 1H), 7.09 (dd, J=7.4, 1.3 Hz, 1H), 7.04 (d, J=8.1 Hz, 1H), 6.62 (d, J=74.4 Hz, 1H), 5.27 (d, J=6.9 Hz, 1H), 5.03 (s, 1H), 4.57 (d, J=7.9 Hz, 1H), 4.26 (d, J=16.7 Hz, 1H), 4.14 (d, J=16.7 Hz, 1H), 3.97-3.93 (m, 1H), 3.89-3.85 (m, 1H), 2.47 (d, J=8.7 Hz, 1H), 2.26-2.20 (m, 1H), 1.58 (d, J=7.0 Hz, 3H). LCMS (ESI) (m/z): 616 (M+H)⁺.

### Scheme 314: Synthesis of (2S,4R)—N—((R)-1-(4-carbamimidoylthiophen-2-yl)ethyl)-4-fluoro-1-((4′-fluoro-[1,1′-biphenyl]-3-carbonyl)glycyl)-4-(fluoromethyl)pyrrolidine-2-carboxamide (Compound 329)

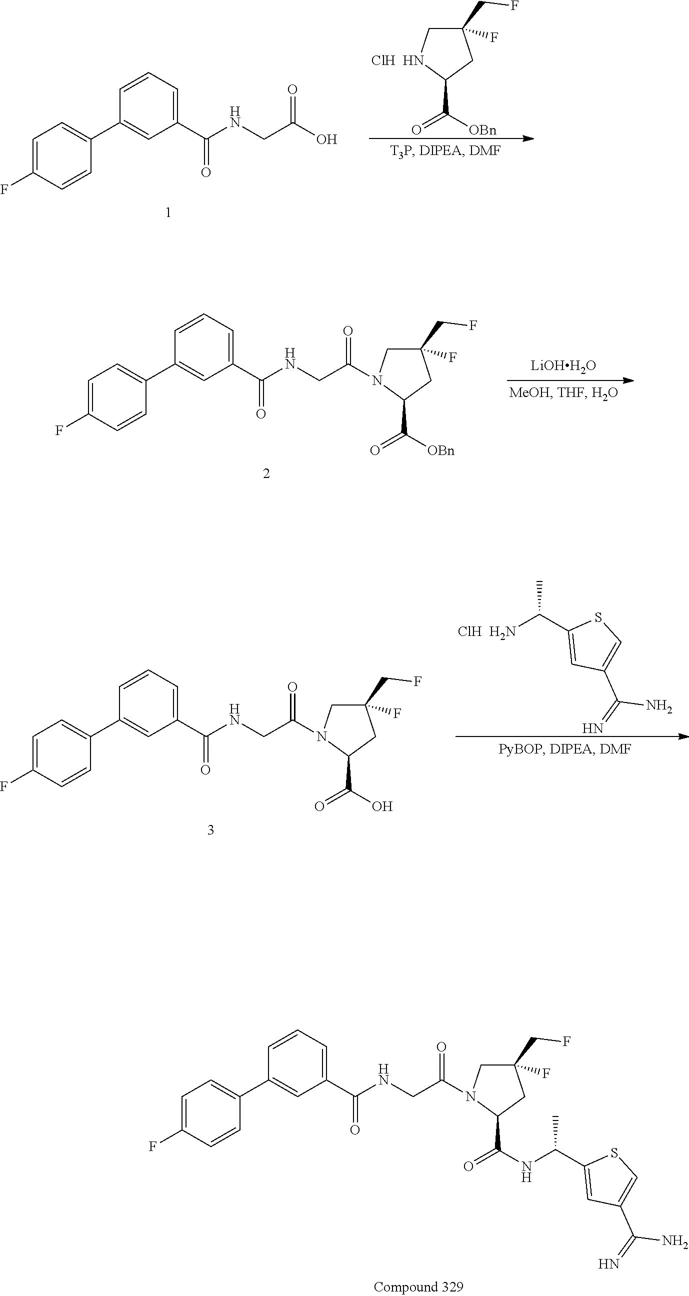

Compound 329 was prepared from (4′-fluoro-[1,1′-biphenyl]-3-carbonyl)glycine, benzyl (2S,4R)-4-fluoro-4-(fluoromethyl)pyrrolidine-2-carboxylate hydrochloride, and (R)-5-(1-aminoethyl)thiophene-3-carboximidamide hydrochloride based on the procedures set forth in Scheme 3. ¹H NMR (400 MHz, CD₃OD) δ 8.22 (dd, J=21.0, 1.6 Hz, 1H), 8.11-8.07 (m, 1H), 7.82 (dd, J=12.4, 7.9 Hz, 2H), 7.72-7.68 (m, 2H), 7.57 (d, J=7.8 Hz, 1H), 7.54 (s, 1H), 7.20 (dd, J=12.2, 5.4 Hz, 2H), 5.27 (q, J=6.9 Hz, 1H), 4.78-4.73 (m, 1H), 4.66 (dd, J=13.7, 5.3 Hz, 2H), 4.34 (d, J=16.7 Hz, 1H), 4.15 (dd, J=18.1, 10.8 Hz, 2H), 3.95 (t, J=13.3 Hz, 1H), 2.66-2.55 (m, 1H), 2.20 (dd, J=12.3, 8.5 Hz, 1H), 1.66-1.58 (m, 3H). LCMS (ESI) (m/z): 572 (M+H)⁺.

### Scheme 315: Synthesis of (2S,4R)—N—((R)-1-(4-carbamimidoylthiophen-2-yl)ethyl)-4-(difluoromethoxy)-1-((4′-fluoro-[1,1′-biphenyl]-3-carbonyl)glycyl)pyrrolidine-2-carboxamide (Compound 330)

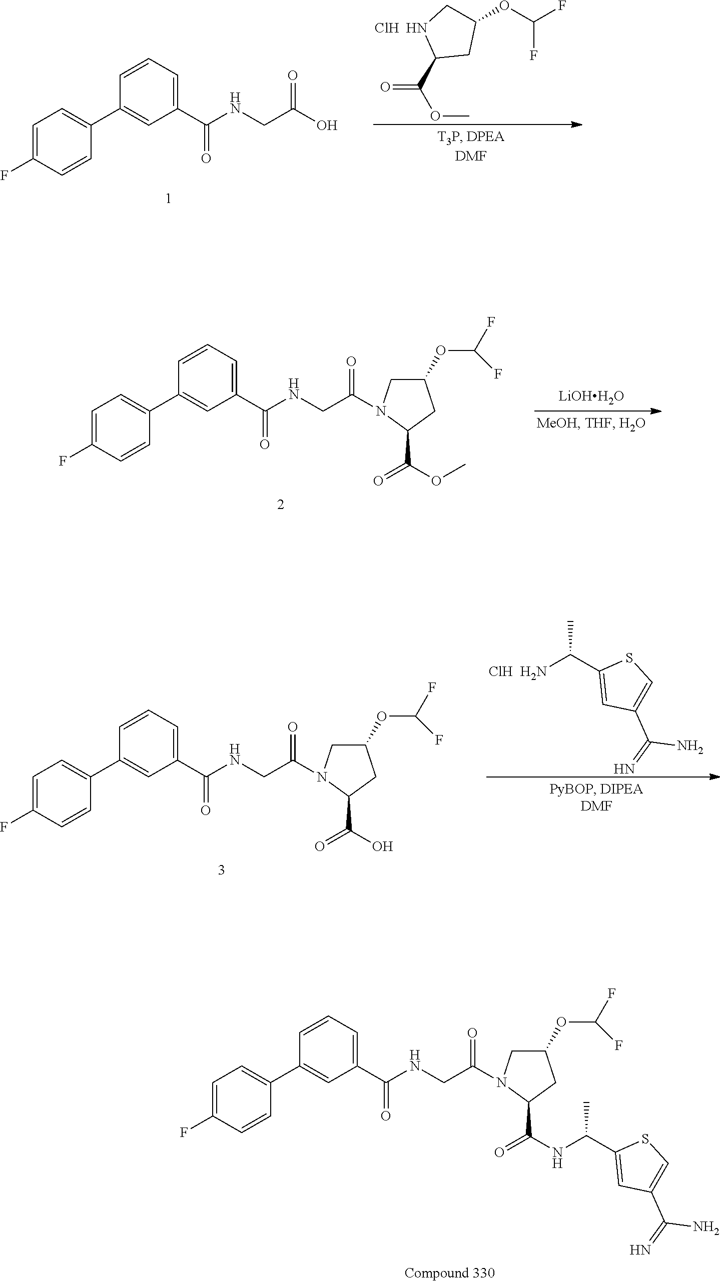

Compound 330 was prepared from (4′-fluoro-[1,1′-biphenyl]-3-carbonyl)glycine, methyl (2S,4R)-4-(difluoromethoxy)pyrrolidine-2-carboxylate hydrochloride, and (R)-5-(1-aminoethyl)thiophene-3-carboximidamide hydrochloride based on the procedures set forth in Scheme 3. ¹H NMR (400 MHz, CD₃OD) δ 8.52 (d, J=5.9 Hz, 1H), 8.22 (dd, J=18.9, 1.6 Hz, 1H), 8.09 (dt, J=12.5, 1.7 Hz, 1H), 7.82 (dd, J=12.8, 8.4 Hz, 2H), 7.72-7.68 (m, 2H), 7.58-7.52 (m, 2H), 7.23-7.18 (m, 2H), 6.51 (td, J=74.4, 16.5 Hz, 1H), 5.37-5.24 (m, 1H), 5.07-4.99 (m, 1H), 4.60 (d, J=7.9 Hz, 1H), 4.31 (d, J=16.7 Hz, 1H), 4.19 (d, J=16.6 Hz, 1H), 3.97 (dd, J=11.5, 4.4 Hz, 1H), 3.90 (d, J=11.5 Hz, 1H), 2.54-2.44 (m, 1H), 2.24 (ddd, J=13.2, 7.9, 5.1 Hz, 1H), 1.62 (dd, J=31.7, 7.0 Hz, 3H). LCMS (ESI) (m/z): 588 (M+H)⁺.

### Scheme 316: Synthesis of (S)-7-((acridine-3-carbonyl)glycyl)-N—((R)-1-(4-carbamimidoylthiophen-2-yl)ethyl)-1,4-dioxa-7-azaspiro[4.4]nonane-8-carboxamide (Compound 331)

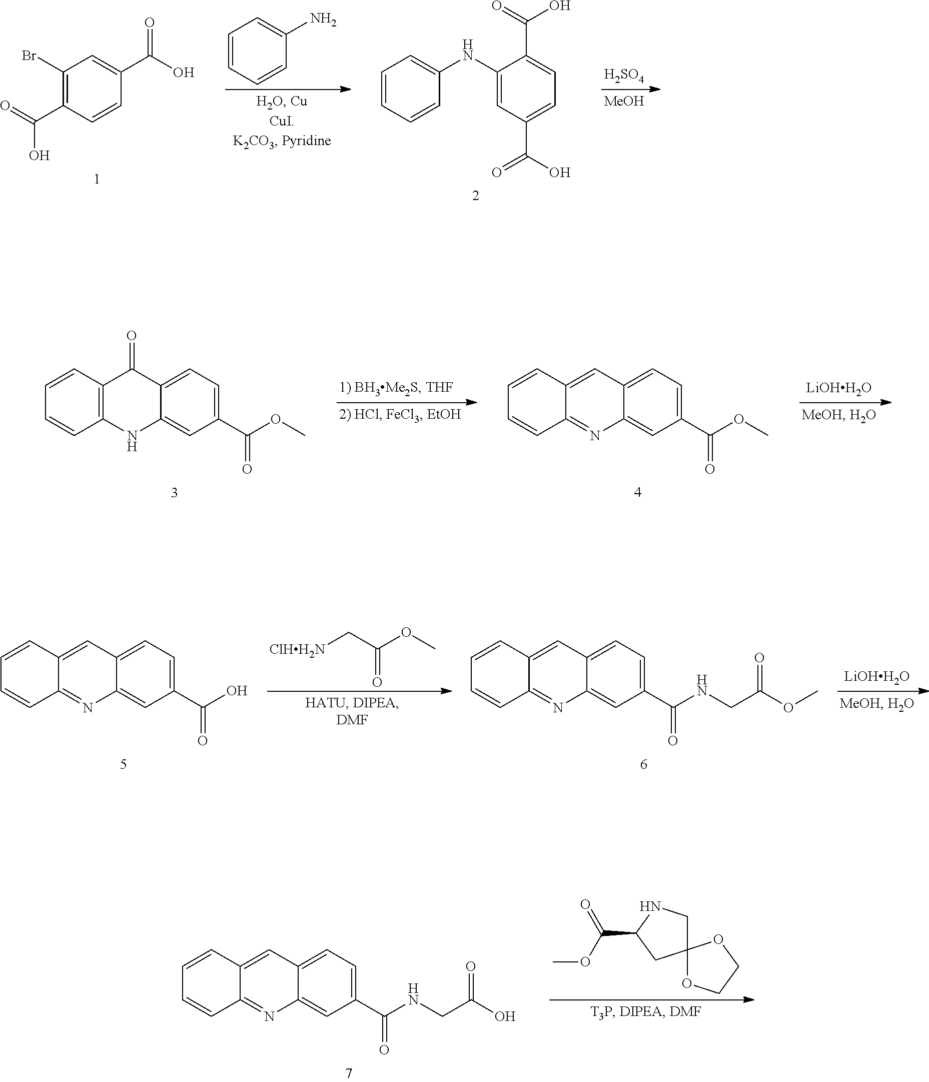

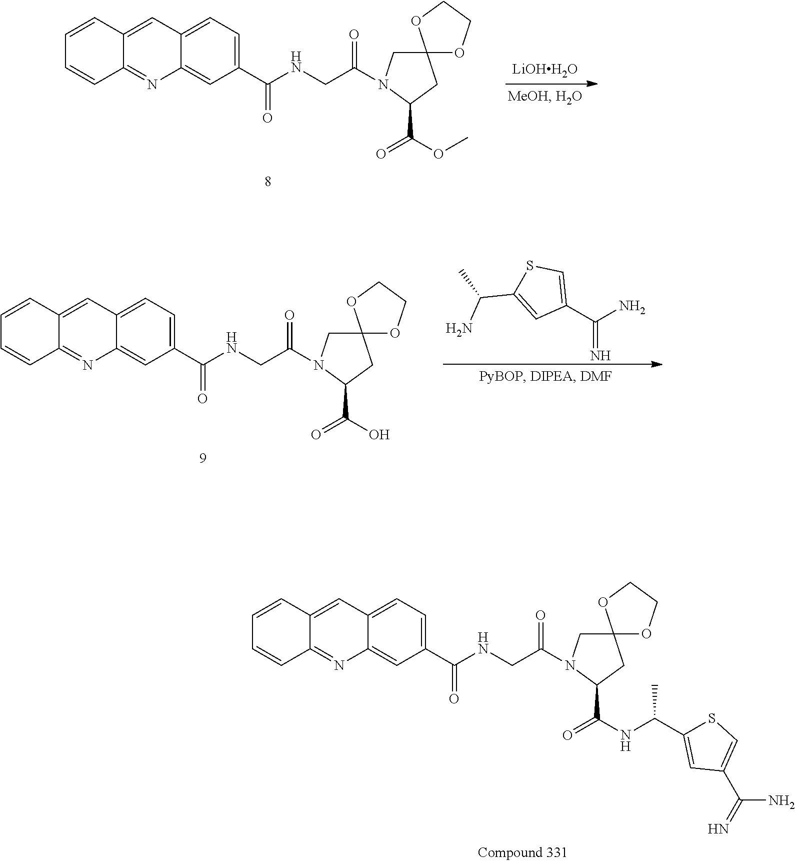

### Step 1: 2-(phenylamino)terephthalic acid

To a solution of K₂CO₃ (2.53 g, 18.37 mmol) in H₂O (60 mL) was added 2-bromoterephthalic acid (3.0 g, 12.24 mmol), aniline (2.3 g, 24.48 mmol), pyridine (0.18 mL, 3.31 mmol), Cu (77.8 mg, 1.84 mmol) and CuI (116 mg, 0.92 mmol). The mixture was stirred at 100° C. for 5 hours. The mixture was acidified with 1 N aq·HCl to pH ˜1 and extracted with EtOAc twice, dried over anhydrous Na₂SO₄, filtered, and concentrated to give 2-(phenylamino)terephthalic acid (1.9 g, yield 60.3%) as a yellow solid. LC/MS (ESI) (m/z): 258 (M+H)⁺.

### Step 2: methyl 9-oxo-9,10-dihydroacridine-3-carboxylate

To a solution of 2-(phenylamino)terephthalic acid (1.9 g, 7.39 mmol) in H₂SO₄ (20 mL) and stirred at 80° C. for 2 hours. The reaction was cooled down to room temperature and MeOH (10 mL) was added. The mixture was stirred at 80° C. for 2 hours. The mixture was basified with aq·NaHCO₃ to pH˜9 and extracted with EtOAc twice. The combined organic layers were dried with anhydrous Na₂SO₄, filtered, and concentrated to dryness, The residue was purified by flash chromatography (0-50% EtOAc in PE) to give methyl 9-oxo-9,10-dihydroacridine-3-carboxylate (300 mg, yield 16.0%) as a yellow solid. LC/MS (ESI) (m/z): 254 (M+H)⁺.

### Step 3: methyl acridine-3-carboxylate

To a solution of methyl 9-oxo-9,10-dihydroacridine-3-carboxylate (200 mg, 0.79 mmol) in THF (5 mL) was added BH₃·Me₂S (0.16 mL, 1.58 mmol, 10 M) at 0° C., and the mixture was stirred at 40° C. for 16 hours. The mixture was quenched with 1 N HCl and extracted with DCM twice. The combined organic layers were dried with anhydrous Na₂SO₄, filtered, and concentrated to dryness. The residue was added EtOH (5 mL), H₂O (1 mL), FeCl₃ (256 mg, 1.58 mmol) and the mixture was stirred at 50° C. for 1 hour. The mixture was basified with aq·NaHCO₃ to pH˜9 and extracted with EtOAc twice. The combined organic layers were dried with anhydrous Na₂SO₄, filtered, and concentrated to give methyl acridine-3-carboxylate (120 mg, yield 64.2%) as a brown oil. LC/MS (ESI) (m/z): 238 (M+H)⁺.

### Step 4: acridine-3-carboxylic acid

To a solution of methyl acridine-3-carboxylate (120 mg, 0.51 mmol) in MeOH (3.0 mL) and water (1.0 mL) was added LiOH·H₂O (42.5 mg, 1.02 mmol) at 0° C. and the mixture was stirred at room temperature for 2 hours. The mixture was acidified with 1 N aq·HCl to pH ˜3 and concentrated under reduced pressure to dryness to give acridine-3-carboxylic acid (100 mg, yield 88.5%) as a yellow solid, which was used directly in the next reaction. LCMS (ESI) (m/z): 224 (M+H)⁺.

### Step 5: methyl (acridine-3-carbonyl)glycinate

To a mixture of acridine-3-carboxylic acid (100 mg, 0.45 mmol) and methyl glycinate hydrochloride (113 mg, 0.90 mmol) in DMF (3 mL) was added HATU (340 mg, 0.90 mmol) and DIPEA (578 mg, 4.50 mmol) and the reaction mixture was stirred at room temperature for 2 hours. The mixture was diluted with EtOAc and washed with water and brine, dried over anhydrous Na₂SO₄, filtered, and concentrated under reduced pressure to dryness. The residue was purified by flash chromatography (silica gel, 0-50% EtOAc in PE) to give methyl (acridine-3-carbonyl)glycinate (70 mg, yield 53.0%) as a colorless oil. LC/MS (ESI) m/z: 295 (M+H)⁺.

### Step 6: (acridine-3-carbonyl)glycine

To a solution of methyl (acridine-3-carbonyl)glycinate (70 mg, 0.24 mmol) in MeOH (3.0 mL) and water (1.0 mL) was added LiOH·H₂O (20 mg, 0.48 mmol) at 0° C. and the mixture was stirred at room temperature for 2 hours. The mixture was acidified with 1 N aq·HCl to pH˜3 and concentrated under reduced pressure to dryness to give acridine-3-carboxylic acid (65 mg, yield 98.5%) as a white solid, which was used directly in the next reaction. LCMS (ESI) (m/z): 281 (M+H)⁺.

### Step 7: methyl (S)-7-((acridine-3-carbonyl)glycyl)-1,4-dioxa-7-azaspiro[4.4]nonane-8-carboxylate

To a mixture of (acridine-3-carbonyl)glycine (65 mg, 0.23 mmol) and methyl (S)-1,4-dioxa-7-azaspiro[4.4]nonane-8-carboxylate (87 mg, 0.46 mmol) in DMF (3 mL) was added DIPEA (178 mg, 1.38 mmol) and T₃P (438 mg, 0.69 mmol, 50% wt. in EtOAc) and the mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-5% MeOH in DCM) to give methyl (S)-7-((acridine-3-carbonyl)glycyl)-1,4-dioxa-7-azaspiro[4.4]nonane-8-carboxylate (60 mg, yield 57.7%) as a brown oil. LC/MS (ESI) m/z: 450 (M+H)⁺.

### Step 8: (S)-7-((acridine-3-carbonyl)glycyl)-1,4-dioxa-7-azaspiro[4.4]nonane-6-carboxylic acid

To a solution of methyl (S)-7-((acridine-3-carbonyl)glycyl)-1,4-dioxa-7-azaspiro[4.4]nonane-8-carboxylate (60 mg, 0.13 mmol) in MeOH (3.0 mL) and water (1.0 mL) was added LiOH·H₂O (11.2 mg, 0.26 mmol) at 0° C. and the mixture was stirred at room temperature for 2 hours. The mixture was acidified with 1 N aq·HCl to pH ˜3 and concentrated under reduced pressure to dryness to give (S)-7-((acridine-3-carbonyl)glycyl)-1,4-dioxa-7-azaspiro[4.4]nonane-8-carboxylic acid (50 mg, yield 86.2%) as a brown solid, which was used directly in the next reaction. LCMS (ESI) (m/z): 436 (M+H)⁺.

### Step 9: (S)-7-((acridine-3-carbonyl)glycyl)-N—((R)-1-(4-carbamimidoylthiophen-2-yl)ethyl)-1,4-dioxa-7-azaspiro[4.4]nonane-6-carboxamide (Compound 331)

To a mixture of (S)-7-((acridine-3-carbonyl)glycyl)-1,4-dioxa-7-azaspiro[4.4]nonane-8-carboxylic acid (50 mg, 0.11 mmol) and (R)-5-(1-aminoethyl)thiophene-3-carboximidamide (37.2 mg, 0.22 mmol) in DMF (3 mL) was added DIPEA (84.1 mg, 0.66 mmol) and PyBOP (85.8 mg, 0.16 mmol) under N₂ atmosphere and the mixture was stirred at room temperature for 2 hours. The mixture was concentrated to dryness under reduced pressure and purified by prep-TLC (DCM:MeOH=10:1) and further purified by prep-HPLC to give Compound 331 (2.0 mg, yield 3.0%) as a white solid. ¹H NMR (400 MHz, CD₃OD) δ 9.13 (s, 1H), 8.68 (s, 1H), 8.51 (s, 1H), 8.26-8.17 (m, 4H), 8.00 (dd, J=8.8, 1.6 Hz, 1H), 7.95-7.90 (m, 1H), 7.71-7.66 (m, 1H), 7.56 (s, 1H), 5.29 (q, J=7.1 Hz, 1H), 4.62-4.59 (m, 1H), 4.27 (q, J=16.6 Hz, 2H), 4.05-3.96 (m, 4H), 3.84 (q, J=10.8 Hz, 2H), 2.45 (dd, J=13.0, 8.9 Hz, 1H), 2.23 (dd, J=13.1, 6.7 Hz, 1H), 1.60 (d, J=7.0 Hz, 3H). LC/MS (ESI) m/z: 587 (M+H)⁺.

### Scheme 317: Synthesis of hexyl ((E)-amino(5-((R)-1-((2S,4R)-1-((9,9-difluoro-9H-fluorene-3-carbonyl)glycyl)-4-fluoro-4-(methoxymethyl)pyrrolidine-2-carboxamido)ethyl)thiophen-3-yl)methylene)carbamate (Compound 249)

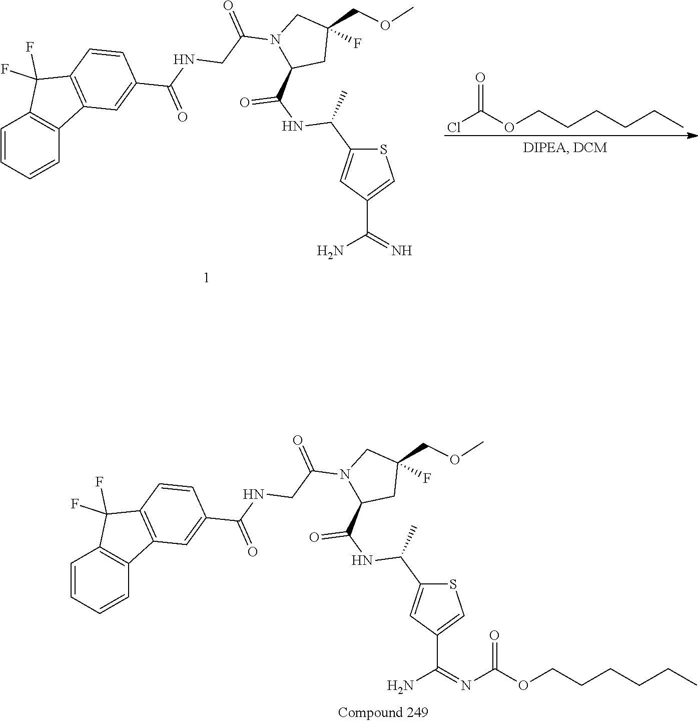

Compound 249 was prepared from (2S,4R)—N—((R)-1-(4-carbamimidoylthiophen-2-yl)ethyl)-1-((9,9-difluoro-9H-fluorene-3-carbonyl)glycyl)-4-fluoro-4-(methoxymethyl)pyrrolidine-2-carboxamide and methyl hexyl carbonochloridate based on the procedures set forth in Scheme 143. ¹H NMR (400 MHz, CD₃OD) δ 8.20-8.13 (m, 1H), 8.11-7.99 (m, 1H), 7.91-7.83 (m, 1H), 7.79-7.62 (m, 4H), 7.56 (s, 1H), 7.43 (t, J=6.8 Hz, 1H), 5.26 (dt, J=20.1, 7.0 Hz, 1H), 4.69-4.60 (m, 1H), 4.39 (d, J=16.8 Hz, 1H), 4.17-3.94 (m, 3H), 3.90-3.81 (m, 2H), 3.72 (dd, J=17.8, 5.7 Hz, 2H), 3.43 (d, J=8.4 Hz, 3H), 2.55 (td, J=14.9, 7.9 Hz, 1H), 2.28-2.11 (m, 1H), 1.61-1.47 (m, 5H), 1.30 (dd, J=16.1, 8.2 Hz, 6H), 0.90 (t, J=6.9 Hz, 3H). LC/MS (ESI) (m/z): 742 (M+H)⁺.

### Scheme 318: Synthesis of (2S,4R)—N—((R)-1-(4-carbamimidoylthiophen-2-yl)ethyl)-1-((dibenzo[b,d]thiophene-3-carbonyl)glycyl)-4-(methylsulfonyl)pyrrolidine-2-carboxamide (Compound 332)

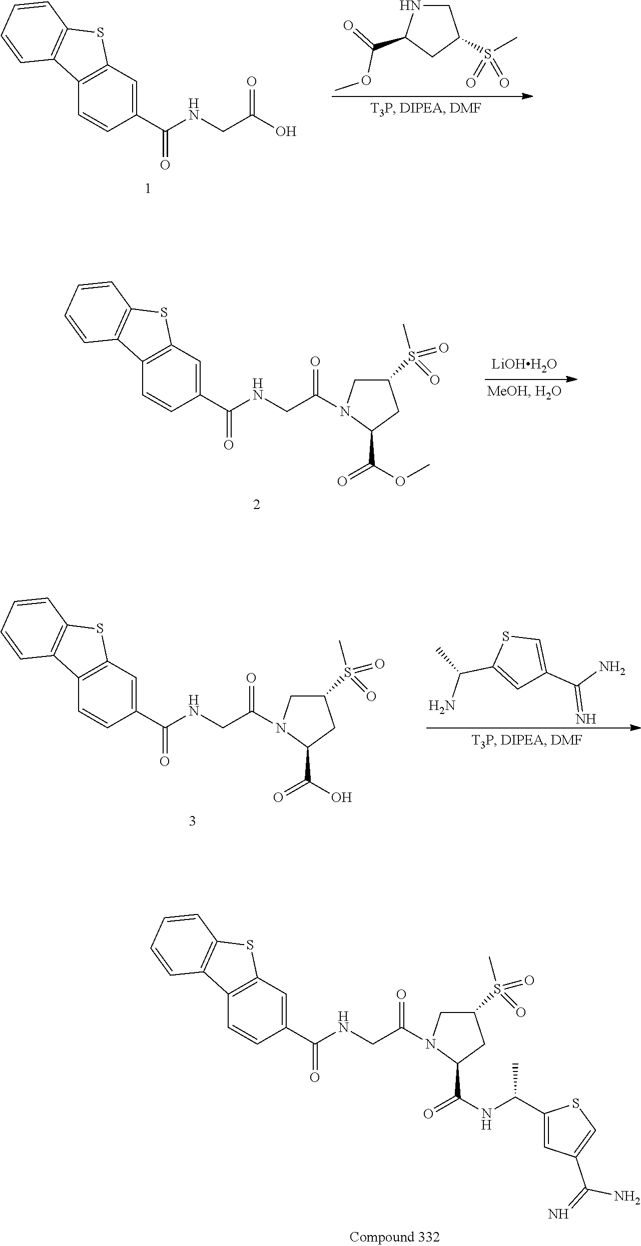

Compound 332 was prepared from (dibenzo[b,d]thiophene-3-carbonyl)glycine, methyl (2S,4R)-4-(methylsulfonyl)pyrrolidine-2-carboxylate, and (R)-5-(1-aminoethyl)thiophene-3-carboximidamide based on the procedures set forth in Scheme 3. ¹H NMR (400 MHz, CD₃OD) δ 8.71 (s, 1H), 8.47 (s, 1H), 8.21 (dd, J=44.3, 19.9 Hz, 2H), 7.90 (s, 3H), 7.57-7.46 (m, 3H), 5.31 (dt, J=19.6, 6.5 Hz, 1H), 4.74 (dd, J=8.6, 4.9 Hz, 1H), 4.32 (d, J=8.3 Hz, 2H), 4.23-4.17 (m, 2H), 4.16-3.92 (m, 1H), 3.08 (d, J=20.6 Hz, 3H), 2.82 (dd, J=30.7, 23.5 Hz, 1H), 2.62-2.40 (m, 1H), 1.69-1.55 (m, 3H). LCMS (ESI) (m/z): 612 (M+H)⁺.

### Scheme 319: (2S,4R)—N—((R)-1-(4-carbamimidoylthiophen-2-yl)ethyl)-4-(methylsulfonyl)-1-((phenoxathiine-3-carbonyl)glycyl)pyrrolidine-2-carboxamide (Compound 333)

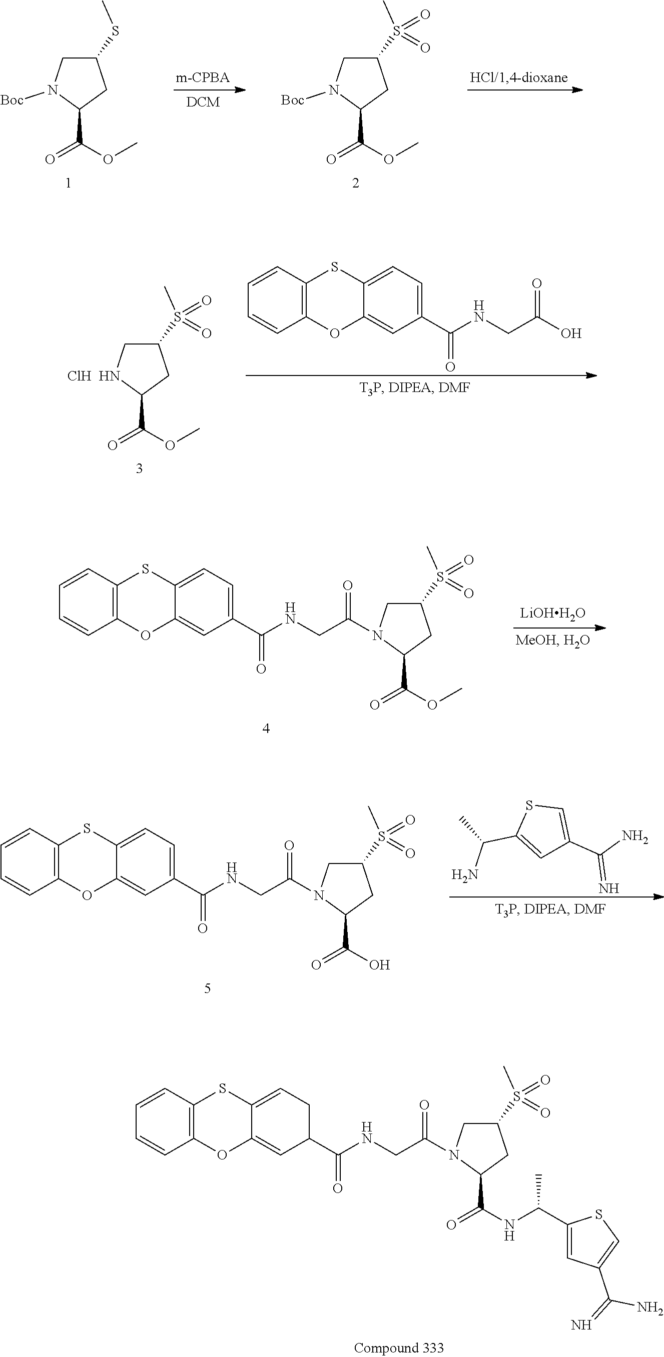

Compound 333 was prepared from 1-(tert-butyl) 2-methyl (2S,4R)-4-(methylthio)pyrrolidine-1,2-dicarboxylate, (phenoxathiine-3-carbonyl)glycine, and (R)-5-(1-aminoethyl)thiophene-3-carboximidamide based on the procedures set forth in Scheme 255. ¹H NMR (400 MHz, CD₃OD) δ 8.50 (s, 1H), 8.25-8.19 (m, 1H), 7.54-7.51 (m, 1H), 7.48 (dt, J=4.0, 1.7 Hz, 2H), 7.23-7.17 (m, 2H), 7.14 (s, 1H), 7.08 (d, J=7.6 Hz, 1H), 7.02 (d, J=8.1 Hz, 1H), 5.26 (d, J=7.0 Hz, 1H), 4.70 (dd, J=8.7, 4.9 Hz, 1H), 4.24 (d, J=4.1 Hz, 1H), 4.16 (d, J=4.4 Hz, 3H), 4.09-3.91 (m, 1H), 3.06 (d, J=18.7 Hz, 3H), 2.76 (d, J=3.4 Hz, 1H), 2.45 (d, J=5.1 Hz, 1H), 1.62 (dd, J=30.8, 7.0 Hz, 3H). LCMS (ESI) (m/z): 628 (M+H)⁺.

### Scheme 320: Synthesis of (2S,4R)—N—((R)-1-(4-carbamimidoylthiophen-2-yl)ethyl)-1-((4′-fluoro-[1,1′-biphenyl]-3-carbonyl)glycyl)-4-(methylsulfonyl)pyrrolidine-2-carboxamide (Compound 334)

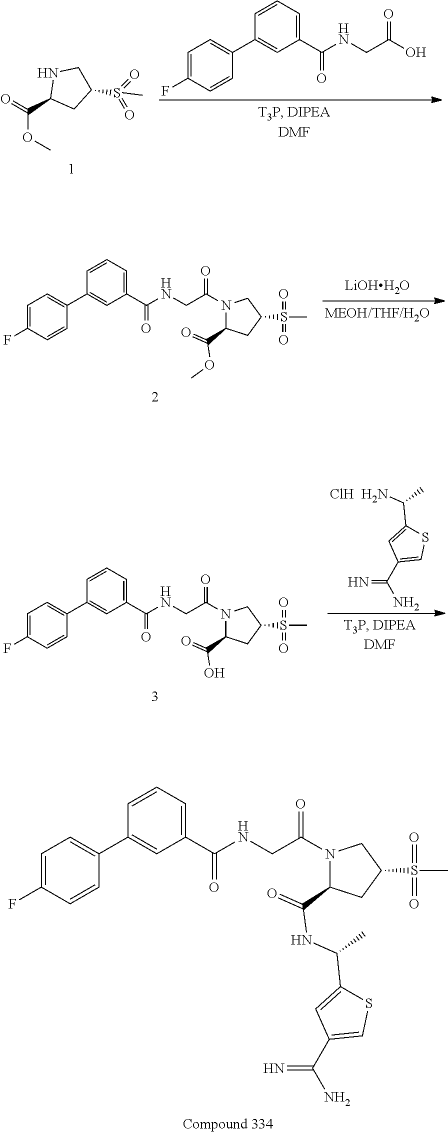

Compound 334 was prepared from methyl (2S,4R)-4-(methylsulfonyl)pyrrolidine-2-carboxylate, (4′-fluoro-[1,1′-biphenyl]-3-carbonyl)glycine, and (R)-5-(1-aminoethyl)thiophene-3-carboximidamide hydrochloride based on the procedures set forth in Scheme 3. ¹H NMR (400 MHz, CD₃OD) δ 8.54 (s, 1H), 8.25-8.07 (m, 2H), 7.82 (dd, J=12.6, 8.0 Hz, 2H), 7.72-7.67 (m, 2H), 7.58-7.48 (m, 2H), 7.21 (ddd, J=8.8, 5.5, 2.1 Hz, 2H), 5.31 (ddd, J=20.9, 9.9, 4.7 Hz, 1H), 4.81-4.57 (m, 1H), 4.32-4.01 (m, 5H), 3.13-3.04 (m, 3H), 2.95-2.65 (m, 1H), 2.61-2.38 (m, 1H), 1.63 (dd, J=31.4, 7.0 Hz, 3H). LC/MS (ESI) m/z: 600 (M+H)⁺.

### Scheme 321: Synthesis of (2S,4R)—N—((R)-1-(4-carbamimidoylthiophen-2-yl)ethyl)-1-((4′-methyl-[1,1′-biphenyl]-3-carbonyl)glycyl)-4-(methylsulfonyl)pyrrolidine-2-carboxamide (Compound 335)

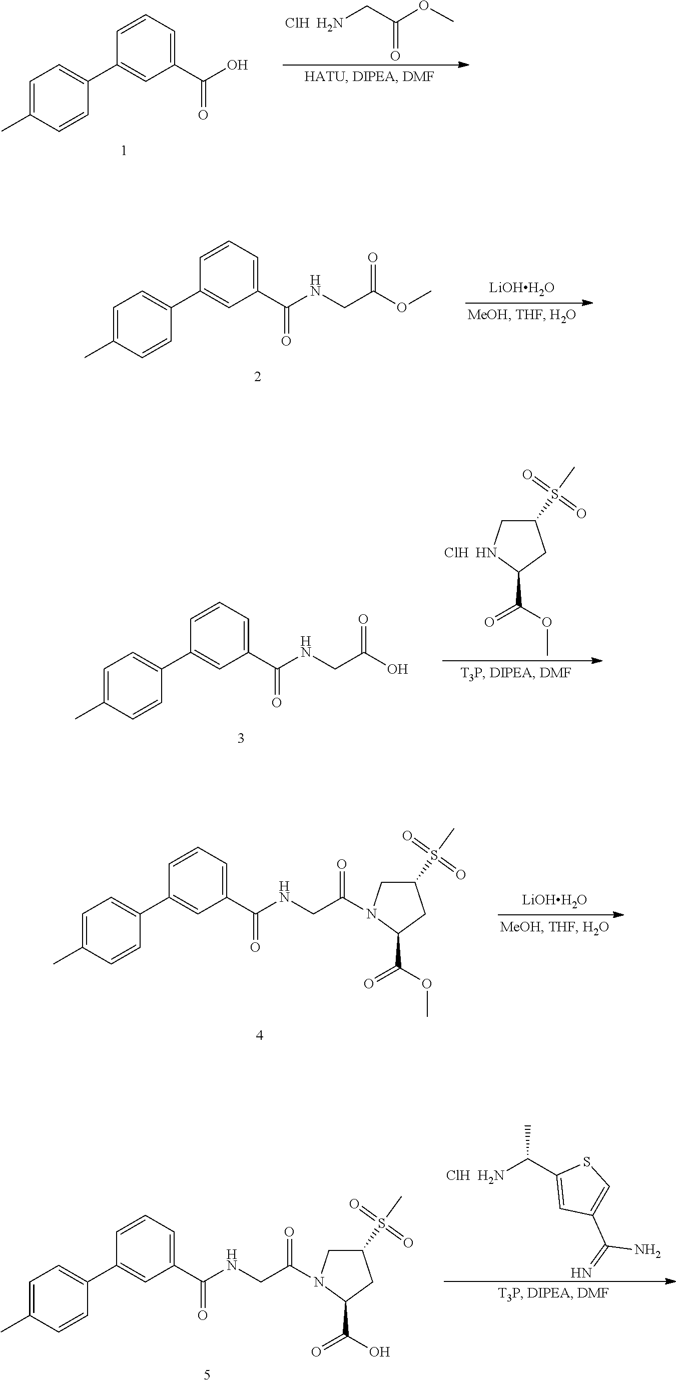

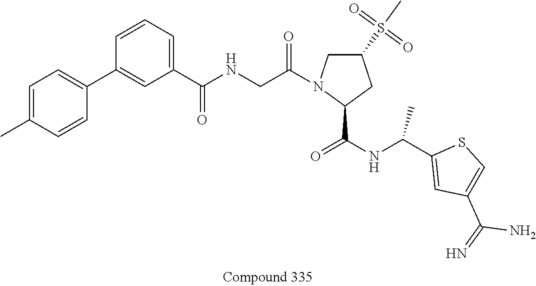 [SMILES: Cc1ccc(-c2cccc(C(=O)NCC(=O)N3C[C@H](S(C)(=O)=O)C[C@H]3C(=O)N[C@H](C)c3cc(C(=N)N)cs3)c2)cc1]

Compound 335 was prepared from 4′-methyl-[1,1′-biphenyl]-3-carboxylic acid, methyl glycinate hydrochloride, methyl (2S,4R)-4-(methylsulfonyl)pyrrolidine-2-carboxylate hydrochloride, and (R)-5-(1-aminoethyl)thiophene-3-carboximidamide hydrochloride based on the procedures set forth in Scheme 66. ¹H NMR (400 MHz, CD₃OD) δ 8.51 (d, J=1.7 Hz, 1H), 8.22 (d, J=22.3 Hz, 1H), 8.09 (d, J=9.0 Hz, 1H), 7.80 (d, J=7.6 Hz, 2H), 7.55 (t, J=7.7 Hz, 3H), 7.49 (s, 1H), 7.28 (d, J=8.0 Hz, 2H), 5.29 (dt, J=20.2, 6.9 Hz, 1H), 4.72 (dd, J=8.7, 4.8 Hz, 1H), 4.36-4.23 (m, 2H), 4.23-4.13 (m, 3H), 3.06 (d, J=18.5 Hz, 3H), 2.88-2.72 (m, 1H), 2.43 (dd, J=13.7, 5.2 Hz, 1H), 2.39 (s, 3H), 1.63 (dd, J=32.0, 7.0 Hz, 3H). LCMS (ESI) (m/z): 596 (M+H)⁺.

### Scheme 322: Synthesis of (2S,4R)—N—((R)-1-(4-carbamimidoylthiophen-2-yl)ethyl)-1-((5,5-dioxidodibenzo[b,d]thiophene-2-carbonyl)glycyl)-4-fluoro-4-(fluoromethyl) pyrrolidine-2-carboxamide (Compound 336)

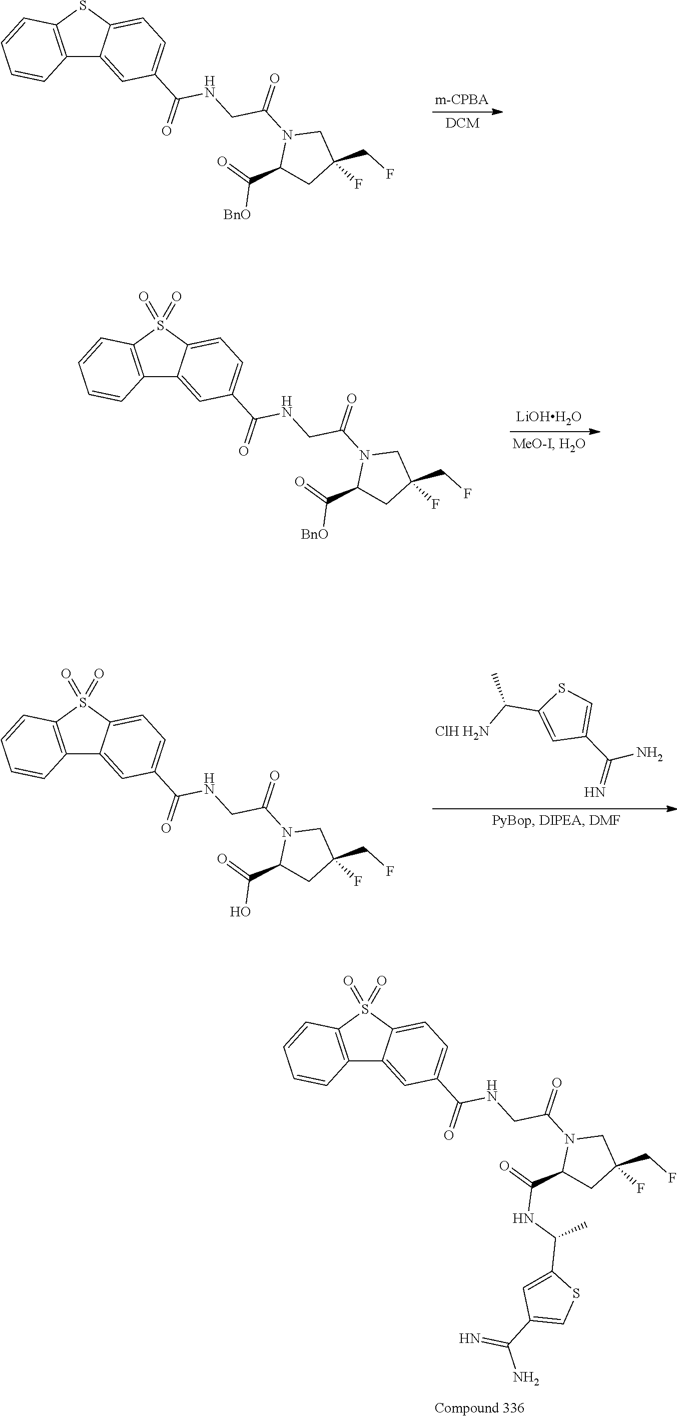

Compound 336 was prepared from benzyl (2S,4R)-1-((dibenzo[b,d]thiophene-2-carbonyl)glycyl)-4-fluoro-4-(fluoromethyl)pyrrolidine-2-carboxylate and (R)-5-(1-aminoethyl)thiophene-3-carboximidamide hydrochloride based on the procedures set forth in Scheme 251. ¹H NMR (400 MHz, CD₃OD) δ 8.49 (s, 2H), 8.19 (s, 1H), 8.06 (d, J=5.5 Hz, 2H), 7.94 (d, J=7.9 Hz, 1H), 7.87 (s, 1H), 7.79 (s, 1H), 7.67 (s, 1H), 7.53 (s, 1H), 5.27 (d, J=6.9 Hz, 1H), 4.76 (s, 1H), 4.70-4.64 (m, 2H), 4.38 (d, J=16.6 Hz, 1H), 4.15 (dd, J=20.2, 10.5 Hz, 2H), 3.98 (dd, J=32.2, 12.3 Hz, 1H), 2.59 (d, J=16.4 Hz, 1H), 2.22 (ddd, J=35.2, 14.4, 9.3 Hz, 1H), 1.58 (d, J=6.8 Hz, 3H). LCMS (ESI) (m/z): 616 (M+H)⁺.

### Scheme 323: Synthesis of (2S,4R)—N—((R)-1-(4-carbamimidoylthiophen-2-yl)ethyl)-4-(difluoromethoxy)-1-((5,5-dioxidodibenzo[b,d]thiophene-2-carbonyl)glycyl) pyrrolidine-2-carboxamide (Compound 337)

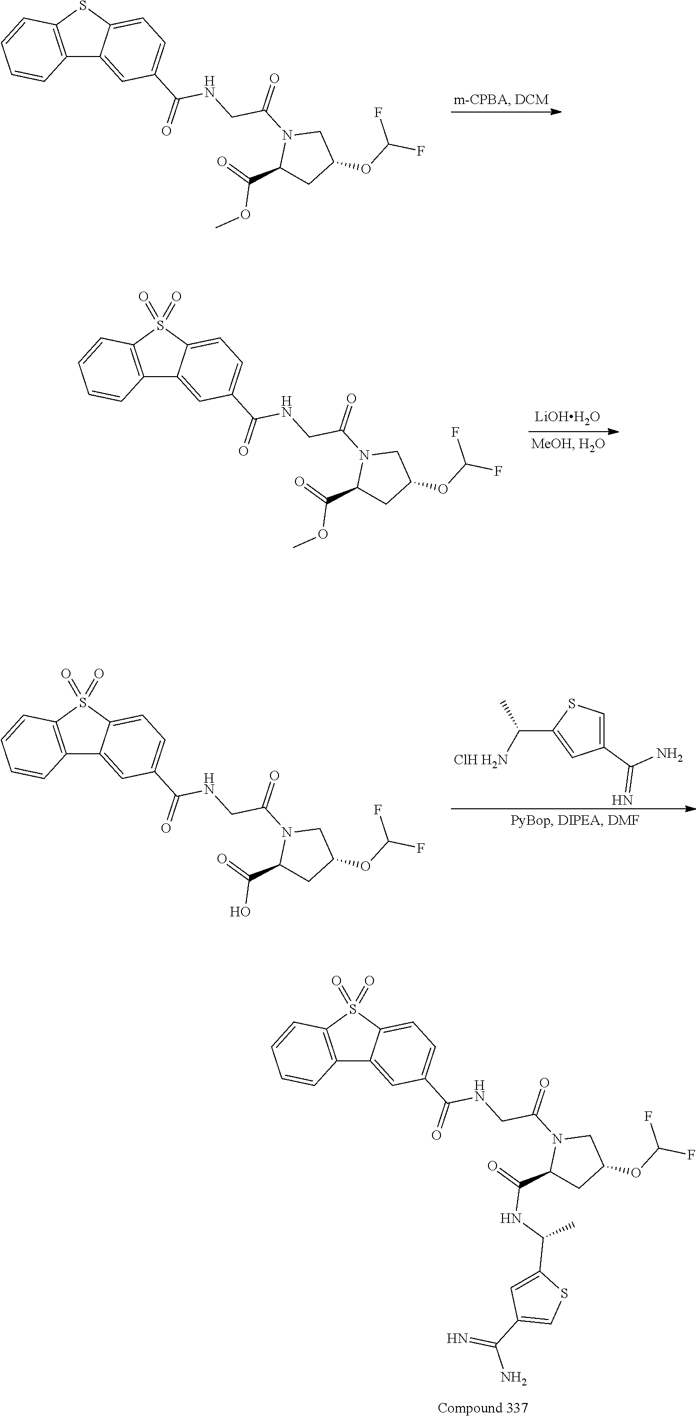

Compound 337 was prepared from methyl (2S,4R)-1-((dibenzo[b,d]thiophene-2-carbonyl)glycyl)-4-(difluoromethoxy)pyrrolidine-2-carboxylate and (R)-5-(1-aminoethyl)thiophene-3-carboximidamide hydrochloride based on the procedures set forth in Scheme 251. ¹H NMR (400 MHz, CD₃OD) δ 8.45 (t, J=6.7 Hz, 2H), 8.20 (dd, J=35.1, 1.5 Hz, 1H), 8.08-7.99 (m, 2H), 7.94-7.84 (m, 2H), 7.77 (dd, J=7.5, 6.6 Hz, 1H), 7.66 (td, J=7.6, 4.0 Hz, 1H), 7.53 (d, J=21.1 Hz, 1H), 6.75-6.29 (m, 1H), 5.30 (dq, J=14.0, 7.1 Hz, 1H), 5.05 (s, 1H), 4.69-4.57 (m, 1H), 4.37-4.12 (m, 2H), 4.01-3.87 (m, 2H), 2.69-2.45 (m, 1H), 2.38-2.18 (m, 1H), 1.62 (dd, J=35.8, 7.0 Hz, 3H). LCMS (ESI) (m/z): 632 (M+H)⁺.

### Scheme 324: Synthesis of N-(2-((2S,4R)-2-(((R)-1-(4-carbamimidoylthiophen-2-yl)ethyl)carbamoyl)-4-(methylsulfonyl)pyrrolidin-1-yl)-2-oxoethyl)-3-fluoro-5-phenylpicolinamide (Compound 339)

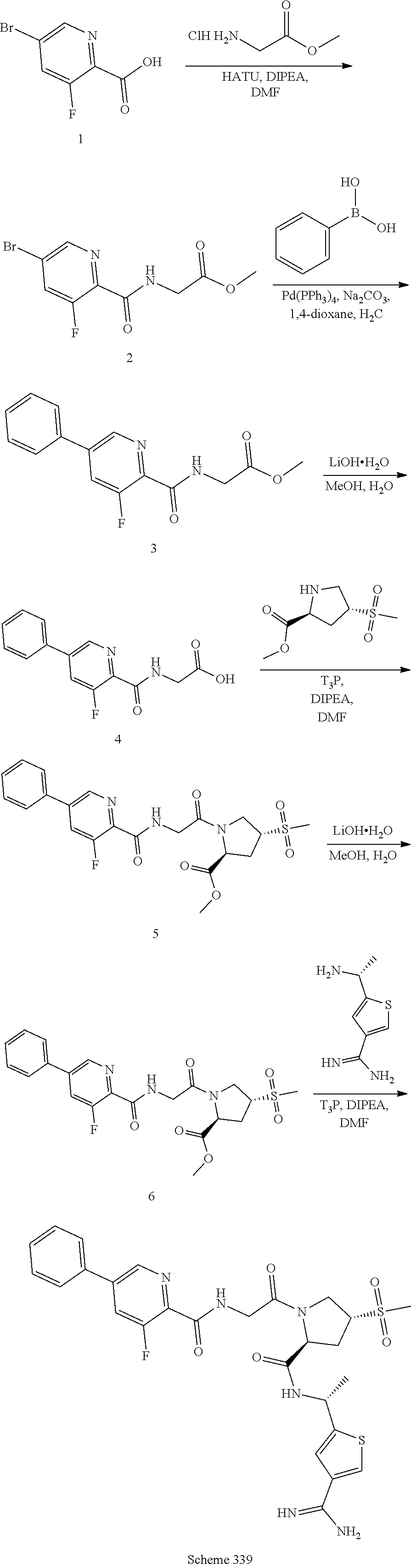

### Step 1: methyl (5-bromo-3-fluoropicolinoyl)glycinate

To a mixture of 5-bromo-3-fluoropicolinic acid (1.0 g, 4.59 mmol) and methyl glycinate hydrochloride (1.16 g, 9.18 mmol) in DMF (10 mL) was added HATU (2.60 g, 6.89 mmol) and DIPEA (4.50 mL, 27.54 mmol) and the reaction mixture was stirred at room temperature for 16 hours. The mixture was diluted with EtOAc and washed with water and brine, dried over anhydrous Na₂SO₄, filtered, and concentrated under reduced pressure to dryness. The residue was purified by flash chromatography (silica gel, 0-30% EtOAc in PE) to give methyl (5-bromo-3-fluoropicolinoyl)glycinate (455 mg, yield 34.2%) as a white solid. LC/MS (ESI) m/z: 291 (M+H)⁺.

### Step 2: methyl (3-fluoro-5-phenylpicolinoyl)glycinate

To a mixture of methyl (5-bromo-3-fluoropicolinoyl)glycinate (250 mg, 0.86 mmol) and phenylboronic acid (137 mg, 1.12 mmol) in 1,4-dioxane (4 mL) and water (1 mL) was added Na₂CO₃ (457 mg, 4.30 mmol), Pd(PPh₃)₄ (100 mg, 0.086 mmol). The mixture was degassed under N₂ atmosphere and stirred at 90° C. overnight. The mixture was diluted with water and extracted with EtOAc twice. The combined organic layers were washed with brine, dried over anhydrous Na₂SO₄, filtered, and concentrated under reduced pressure to dryness. The residue was purified by column chromatography (silica gel, 0-50% EtOAc in PE) to give methyl (3-fluoro-5-phenylpicolinoyl)glycinate (200 mg, yield 80.6%) as a white solid. LCMS (ESI) m/z: 289 (M+H)⁺.

### Step 3: (3-fluoro-5-phenylpicolinoyl)glycine

To a solution of methyl (3-fluoro-5-phenylpicolinoyl)glycinate (200 mg, 0.69 mmol) in MeOH (3 mL) and water (1 mL) was added LiOH·H₂O (58 mg, 1.38 mmol) at 0° C. and the mixture was stirred at room temperature for 2 hours. The mixture was acidified with 1 N aq·HCl to pH ˜4 and extracted with EtOAc twice. The combined organic layers were washed with brine, dried over anhydrous Na₂SO₄, filtered, and concentrated to give (3-fluoro-5-phenylpicolinoyl)glycine (162 mg, yield 85.3%) as a brown solid. LCMS (ESI) (m/z): 275 (M+H)⁺.

### Step 4: methyl (2S,4R)-1-((3-fluoro-5-phenylpicolinoyl)glycyl)-4-(methylsulfonyl) pyrrolidine-2-carboxylate

To a mixture of (3-fluoro-5-phenylpicolinoyl)glycine (50 mg, 0.18 mmol) and methyl (2S,4R)-4-(methylsulfonyl)pyrrolidine-2-carboxylate hydrochloride (75 mg, 0.36 mmol) in DMF (3 mL) was added T₃P (348 mg, 0.54 mmol, 50% wt. in EtOAc) and DIPEA (0.18 mL, 1.08 mmol) and the reaction mixture was stirred at room temperature for 2 hours. The mixture was diluted with EtOAc and washed with water and brine, dried over anhydrous Na₂SO₄, filtered, and concentrated under reduced pressure to dryness. The residue was purified by flash chromatography (silica gel, 0-80% EtOAc in PE) to give methyl (2S,4R)-1-((3-fluoro-5-phenylpicolinoyl)glycyl)-4-(methylsulfonyl)pyrrolidine-2-carboxylate (70 mg, yield 83.3%) as a colorless oil. LC/MS (ESI) m/z: 464 (M+H)⁺.

### Step 5: (2S,4R)-1-((3-fluoro-5-phenylpicolinoyl)glycyl)-4-(methylsulfonyl)pyrrolidine-2-carboxylic acid

To a solution of methyl (2S,4R)-1-((3-fluoro-5-phenylpicolinoyl)glycyl)-4-(methylsulfonyl)pyrrolidine-2-carboxylate (70 mg, 0.15 mmol) in MeOH (3.0 mL) and water (1.0 mL) was added LiOH·H₂O (13 mg, 0.30 mmol) at 0° C. and the mixture was stirred at room temperature for 2 hours. The mixture was acidified with 1 N aq·HCl to pH˜3 and extracted with EtOAc twice. The combined organic layers were washed with brine, dried over anhydrous Na₂SO₄, filtered, and concentrated to give (2S,4R)-1-((3-fluoro-5-phenylpicolinoyl)glycyl)-4-(methylsulfonyl)pyrrolidine-2-carboxylic acid (50 mg, yield 73.5%) as a white solid. LCMS (ESI) (m/z): 450 (M+H)⁺.

### Step 6: N-(2-((2S,4R)-2-(((R)-1-(4-carbamimidoylthiophen-2-yl)ethyl)carbamoyl)-4-(methylsulfonyl)pyrrolidin-1-yl)-2-oxoethyl)-3-fluoro-5-phenylpicolinamide (Compound 339)

To a mixture of (2S,4R)-1-((3-fluoro-5-phenylpicolinoyl)glycyl)-4-(methylsulfonyl) pyrrolidine-2-carboxylic acid (50 mg, 0.11 mmol) and (R)-5-(1-aminoethyl)thiophene-3-carboximidamide (38 mg, 0.22 mmol) in DMF (3 mL) was added DIPEA (86 mg, 0.66 mmol) and T₃P (106 mg, 0.17 mmol, 50% wt. in EtOAc) and the mixture was stirred at room temperature for 2 hours. The mixture was quenched with aq·NaHCO₃ and extracted with CHCl₃/i-PrOH(3/1) five times, dried over anhydrous Na₂SO₄, filtered, and concentrated under reduced pressure to dryness. The residue was purified by prep-TLC (DCM:MeOH=10:1) and further purified by prep-HPLC to give Compound 339 (10.0 mg, yield 14.9%) as a white solid. ¹H NMR (400 MHz, CD₃OD) δ 8.80 (s, 1H), 8.53 (s, 1H), 8.24 (dd, J=10.9, 1.5 Hz, 1H), 8.02 (dd, J=12.1, 1.6 Hz, 1H), 7.77 (d, J=7.0 Hz, 2H), 7.58-7.48 (m, 4H), 5.29 (dt, J=20.8, 7.0 Hz, 1H), 4.72 (dd, J=8.5, 4.9 Hz, 1H), 4.35-3.97 (m, 5H), 3.07 (d, J=17.5 Hz, 3H), 2.93-2.71 (m, 1H), 2.59-2.39 (m, 1H), 1.60 (d, J=7.0 Hz, 3H). LC/MS (ESI) m/z: 601 (M+H)⁺.

### Scheme 325: Synthesis of N-(2-((2S,4R)-2-(((R)-1-(4-carbamimidoylthiophen-2-yl)ethyl)carbamoyl)-4-(methylsulfonyl)pyrrolidin-1-yl)-2-oxoethyl)-9H-carbazole-3-carboxamide (Compound 340)

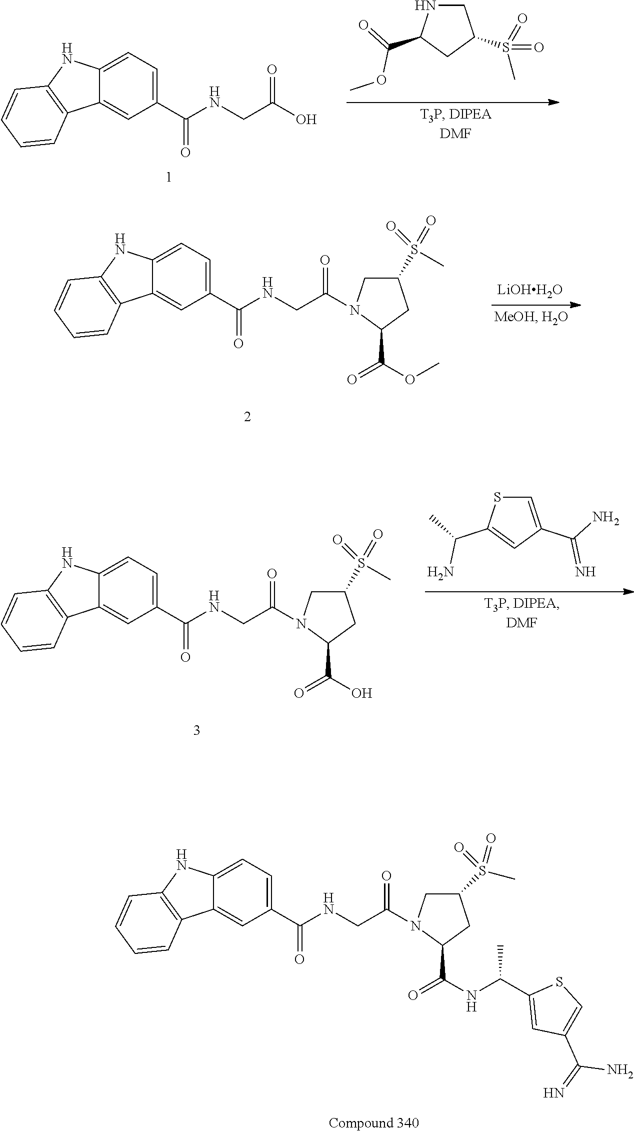

Compound 340 was prepared from (9H-carbazole-3-carbonyl)glycine, methyl (2S,4R)-4-(methylsulfonyl)pyrrolidine-2-carboxylate, and (R)-5-(1-aminoethyl)thiophene-3-carboximidamide based on the procedures set forth in Scheme 66. ¹H NMR (400 MHz, CD₃OD) δ 8.65 (s, 1H), 8.48 (s, 1H), 8.25-8.17 (m, 1H), 8.09 (d, J=7.6 Hz, 1H), 7.91 (d, J=1.5 Hz, 1H), 7.57-7.47 (m, 3H), 7.43 (d, J=0.9 Hz, 1H), 7.22 (t, J=7.4 Hz, 1H), 5.27 (d, J=6.9 Hz, 1H), 4.74 (dd, J=8.7, 4.9 Hz, 1H), 4.32 (s, 2H), 4.17 (d, J=7.1 Hz, 2H), 4.07 (s, 1H), 3.07 (d, J=18.5 Hz, 3H), 2.90-2.74 (m, 1H), 2.44 (dd, J=10.7, 4.2 Hz, 1H), 1.63 (dd, J=32.4, 7.0 Hz, 3H). LCMS (ESI) (m/z): 595 (M+H)⁺.

### Scheme 326: Synthesis of N-(2-((2S,4R)-2-(((R)-1-(4-carbamimidoylthiophen-2-yl)ethyl)carbamoyl)-4-fluoro-4-(fluoromethyl)pyrrolidin-1-yl)-2-oxoethyl)-9H-carbazole-3-carboxamide (Compound 341)

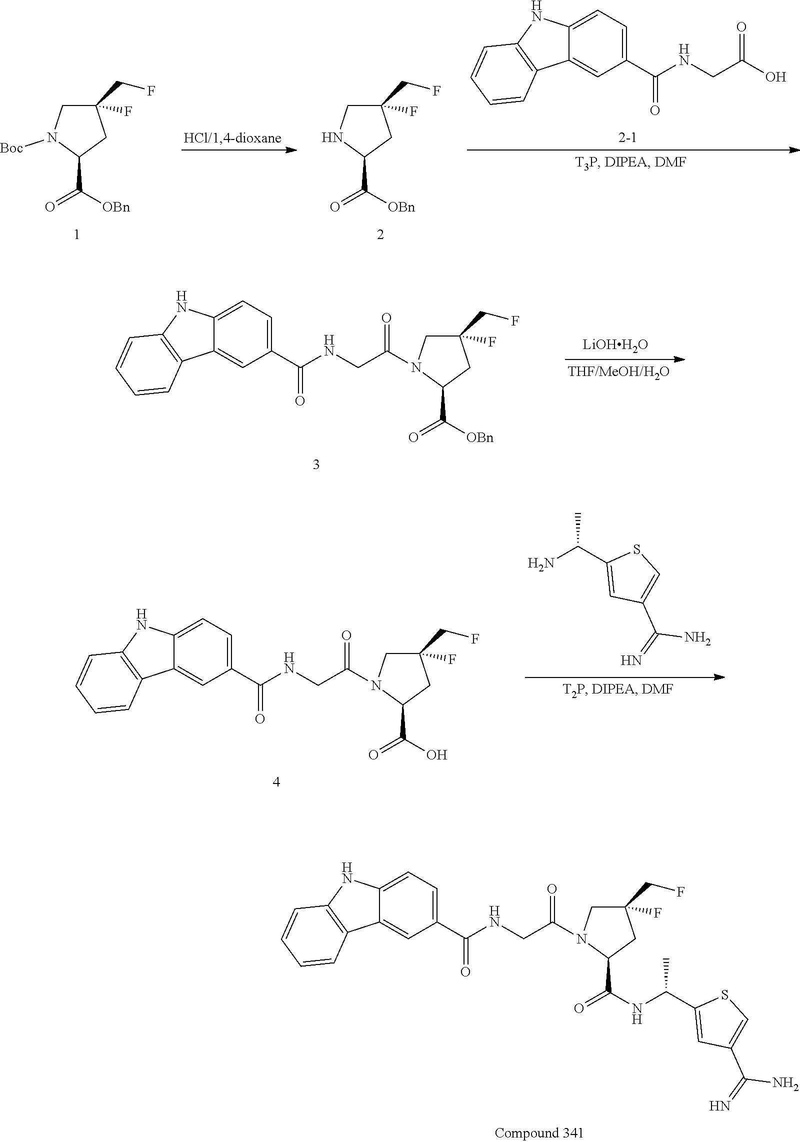

Compound 341 was prepared from 2-benzyl 1-(tert-butyl) (2S,4R)-4-fluoro-4-(fluoromethyl)pyrrolidine-1,2-dicarboxylate, (9H-carbazole-3-carbonyl)glycine, and (R)-5-(1-aminoethyl)thiophene-3-carboximidamide based on the procedures set forth in Scheme 3. ¹H NMR (400 MHz, CD₃OD) δ 8.65 (dd, J=8.9, 1.5 Hz, 1H), 8.52 (s, 1H), 8.23 (dd, J=22.0, 1.6 Hz, 1H), 8.12 (dd, J=12.1, 7.8 Hz, 1H), 7.96-7.89 (m, 1H), 7.57 (dd, J=6.8, 5.4 Hz, 1H), 7.50 (d, J=8.7 Hz, 2H), 7.46-7.41 (m, 1H), 7.25-7.20 (m, 1H), 5.28 (q, J=6.8 Hz, 1H), 4.79-4.64 (m, 3H), 4.24 (dt, J=18.9, 15.2 Hz, 3H), 4.04-3.91 (m, 1H), 2.87-2.56 (m, 1H), 2.23 (ddd, J=35.0, 14.3, 9.4 Hz, 1H), 1.63 (dd, J=32.1, 7.0 Hz, 3H). LC/MS (ESI) m/z:567 (M+H)⁺.

### Scheme 327: Synthesis of (2S,4R)—N—((R)-1-(4-carbamimidoylthiophen-2-yl)ethyl)-1-((5,5-dioxidodibenzo[b,d]thiophene-2-carbonyl)glycyl)-4-(methylsulfonyl) pyrrolidine-2-carboxamide (Compound 342)

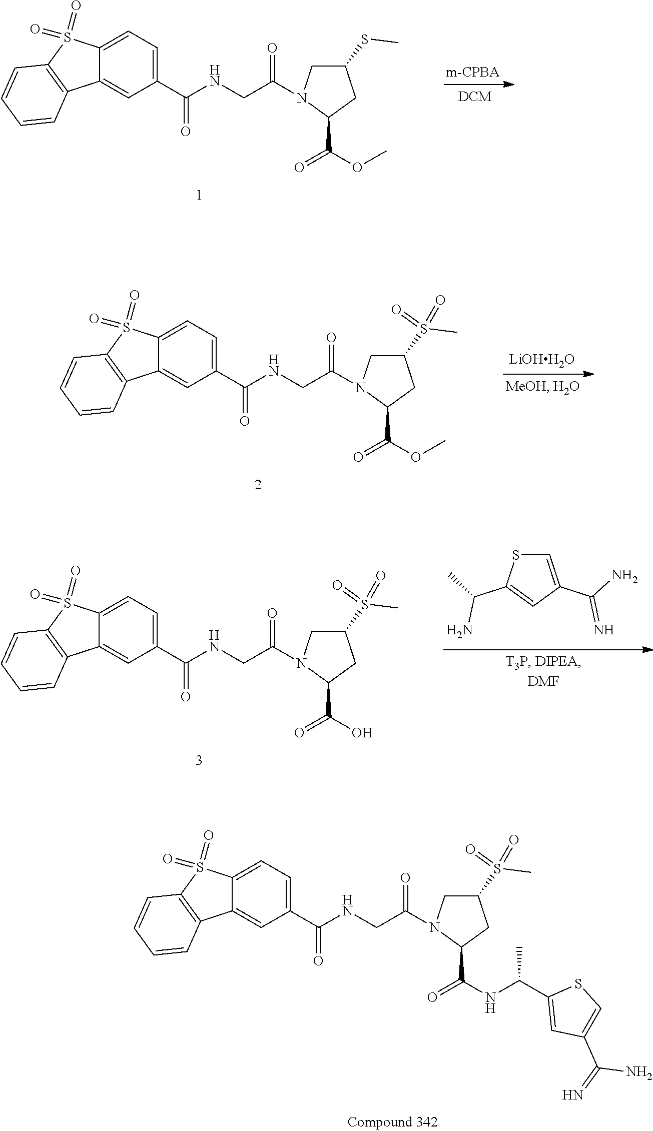

Compound 342 was prepared from methyl (2S,4R)-1-((dibenzo[b,d]thiophene-2-carbonyl)glycyl)-4-(methylsulfonyl)pyrrolidine-2-carboxylate and (R)-5-(1-aminoethyl)thiophene-3-carboximidamide based on the procedures set forth in Scheme 229. ¹H NMR (400 MHz, CD₃OD) δ 8.47 (s, 2H), 8.21 (dd, J=27.1, 1.5 Hz, 1H), 8.10-8.01 (m, 2H), 7.93 (dd, J=8.0, 5.3 Hz, 1H), 7.87 (d, J=7.6 Hz, 1H), 7.78 (dd, J=7.6, 6.7 Hz, 1H), 7.69-7.64 (m, 1H), 7.56-7.47 (m, 1H), 5.28 (q, J=7.0 Hz, 1H), 4.72 (dd, J=8.6, 5.0 Hz, 1H), 4.36-4.05 (m, 5H), 3.08 (d, J=19.7 Hz, 3H), 2.95-2.75 (m, 1H), 2.43 (dt, J=13.4, 6.2 Hz, 1H), 1.59 (d, J=7.0 Hz, 3H). LCMS (ESI) (m/z): 644 (M+H)⁺.

### Scheme 328: Synthesis of (2S,4R)—N—((R)-1-(4-carbamimidoylthiophen-2-yl)ethyl)-1-((dibenzo[b,d]thiophene-3-carbonyl)glycyl)-4-fluoro-4-(methoxymethyl)pyrrolidine-2-carboxamide (Compound 343)

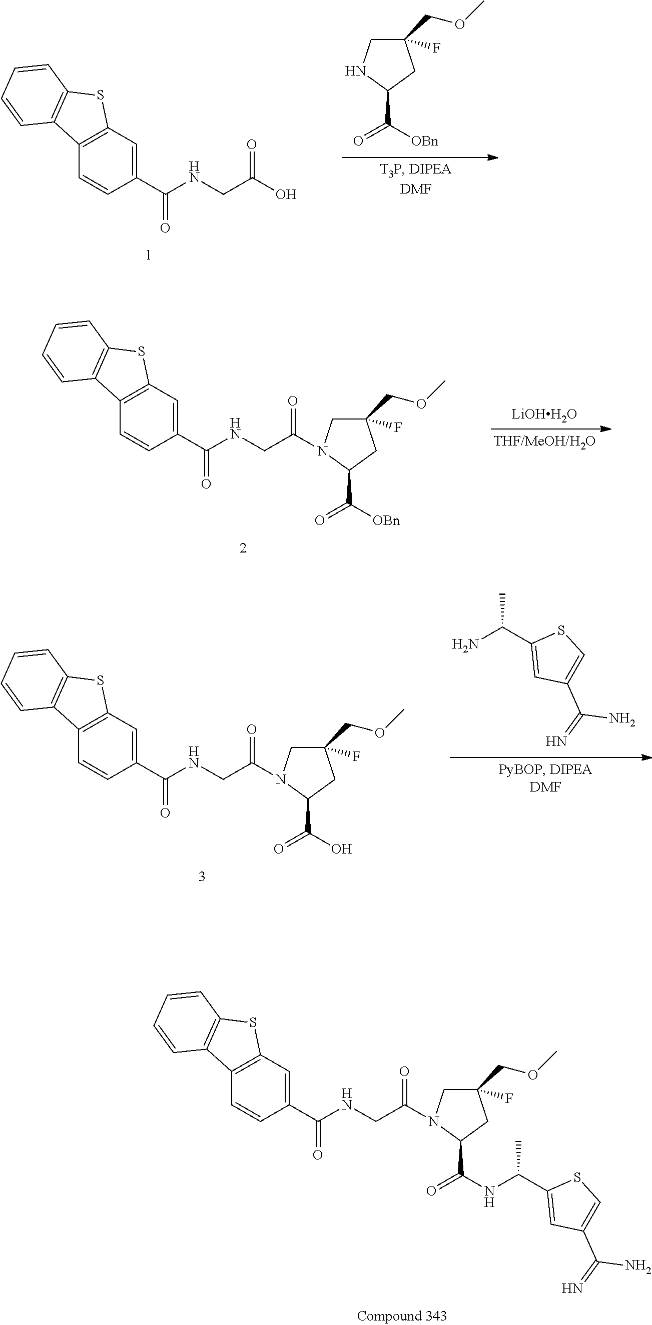

Compound 343 was prepared from (dibenzo[b,d]thiophene-3-carbonyl)glycine, benzyl (2S,4R)-4-fluoro-4-(methoxymethyl)pyrrolidine-2-carboxylate, and (R)-5-(1-aminoethyl)thiophene-3-carboximidamide based on the procedures set forth in Scheme 3. ¹H NMR (400 MHz, CD₃OD) δ 8.54 (s, 1H), 8.41 (d, J=1.2 Hz, 1H), 8.35-8.30 (m, 2H), 8.23 (dd, J=20.2, 1.6 Hz, 1H), 7.98-7.92 (m, 2H), 7.58-7.52 (m, 3H), 5.33-5.23 (m, 1H), 4.67-4.60 (m, 1H), 4.37-4.03 (m, 3H), 3.98-3.86 (m, 1H), 3.75-3.66 (m, 2H), 3.43 (d, J=5.2 Hz, 3H), 2.79-2.50 (m, 1H), 2.32-2.11 (m, 1H), 1.63 (dd, J=33.2, 7.0 Hz, 3H). LCMS (ESI) (m/z): 596 (M+H)⁺.

### Scheme 329: Synthesis of (2S,4R)—N—((R)-1-(4-carbamimidoylthiophen-2-yl)ethyl)-1-((9,9-dimethyl-9H-fluorene-2-carbonyl)glycyl)-4-fluoro-4-(methoxymethyl) pyrrolidine-2-carboxamide (Compound 344)

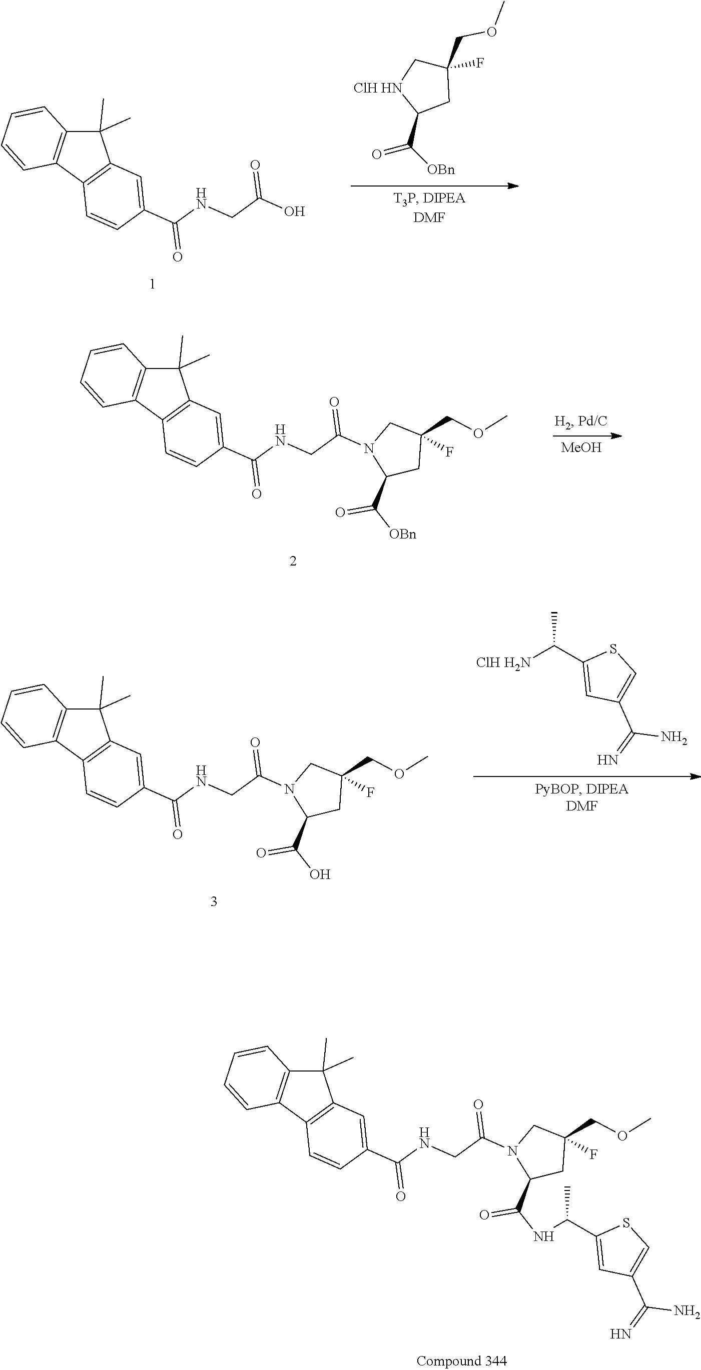

Compound 344 was prepared from methyl (9,9-dimethyl-9H-fluorene-2-carbonyl)glycine, benzyl (2S,4R)-4-fluoro-4-(methoxymethyl)pyrrolidine-2-carboxylate hydrochloride, and (R)-5-(1-aminoethyl)thiophene-3-carboximidamide hydrochloride based on the procedures set forth in Scheme 12. ¹H NMR (400 MHz, CD₃OD) δ 8.53 (s, 1H), 8.23 (dd, J=17.0, 1.6 Hz, 1H), 7.99 (d, J=10.0 Hz, 1H), 7.87-7.81 (m, 3H), 7.56-7.49 (m, 2H), 7.39-7.35 (m, 2H), 5.31 (dt, J=20.7, 7.3 Hz, 1H), 4.66-4.58 (m, 1H), 4.33 (d, J=16.6 Hz, 1H), 4.15 (d, J=16.7 Hz, 1H), 4.12-4.03 (m, 1H), 3.97-3.85 (m, 1H), 3.74-3.66 (m, 2H), 3.43 (d, J=4.8 Hz, 3H), 2.55 (td, J=14.6, 7.2 Hz, 1H), 2.19 (ddd, J=36.9, 14.0, 9.6 Hz, 1H), 1.61 (t, J=16.3 Hz, 3H), 1.51 (d, J=3.1 Hz, 6H). LCMS (ESI) (m/z): 606 (M+H)⁺.

### Scheme 330: Synthesis of (2S,4R)—N—((R)-1-(4-carbamimidoylthiophen-2-yl)ethyl)-4-(difluoromethoxy)-1-((9,9-dimethyl-9H-fluorene-2-carbonyl)glycyl)pyrrolidine-2-carboxamide (Compound 248)

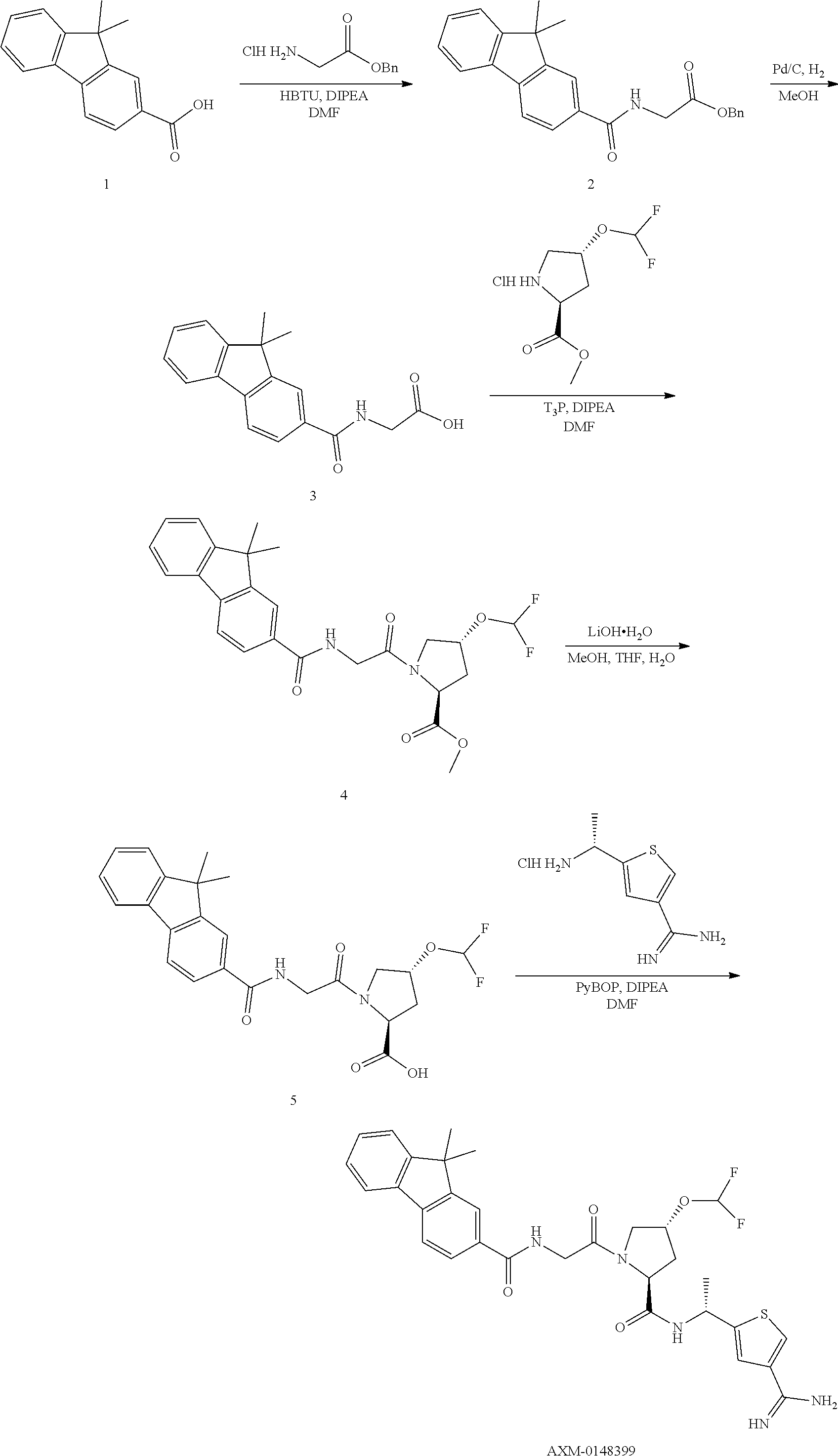

Compound 330 was prepared from 9,9-dimethyl-9H-fluorene-2-carboxylic acid, benzyl glycinate hydrochloride, methyl (2S,4R)-4-(difluoromethoxy)pyrrolidine-2-carboxylate hydrochloride, and (R)-5-(1-aminoethyl) thiophene-3-carboximidamide hydrochloride based on the procedures set forth in Scheme 172. ¹H NMR (400 MHz, CD₃OD) δ 8.50 (s, 1H), 8.26-8.20 (m, 1H), 8.00 (d, J=10.0 Hz, 1H), 7.85 (dt, J=11.1, 4.9 Hz, 3H), 7.53 (dd, J=12.7, 8.2 Hz, 2H), 7.39-7.35 (m, 2H), 6.51 (td, J=74.5, 16.5 Hz, 1H), 5.31 (dt, J=13.2, 6.3 Hz, 1H), 5.04 (s, 1H), 4.59 (d, J=7.7 Hz, 1H), 4.31 (d, J=16.7 Hz, 1H), 4.19 (d, J=16.6 Hz, 1H), 3.98 (dd, J=11.5, 4.4 Hz, 1H), 3.92 (s, 1H), 2.58-2.41 (m, 1H), 2.30-2.18 (m, 1H), 1.61 (t, J=16.1 Hz, 3H), 1.51 (d, J=3.5 Hz, 6H). LCMS (ESI) (m/z): 610 (M+H)⁺.

### Scheme 331: Synthesis of (2S,4R)—N—((R)-1-(4-carbamimidoylthiophen-2-yl)ethyl)-1-((dibenzo[b,d]thiophene-3-carbonyl)glycyl)-4-fluoro-4-(fluoromethyl)pyrrolidine-2-carboxamide (Compound 349)

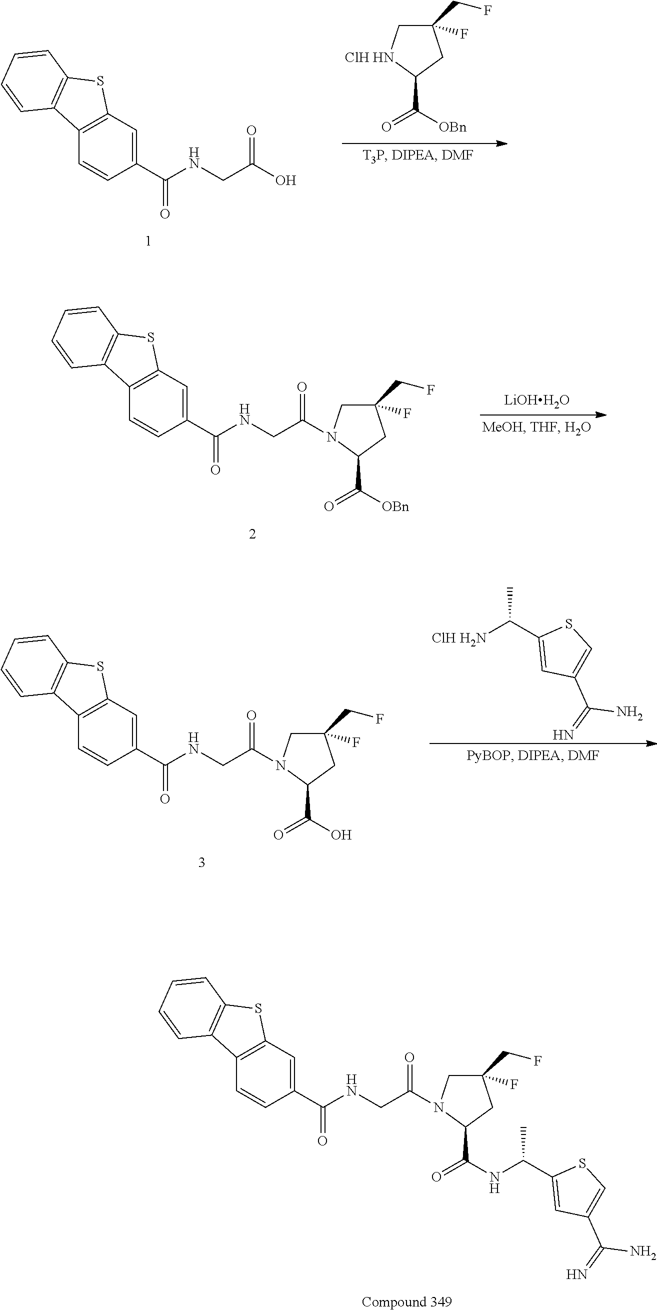

Compound 349 was prepared from (dibenzo[b,d]thiophene-3-carbonyl)glycine, benzyl (2S,4R)-4-fluoro-4-(fluoromethyl)pyrrolidine-2-carboxylate hydrochloride, and (R)-5-(1-aminoethyl)thiophene-3-carboximidamide hydrochloride based on the procedures set forth in Scheme 3. ¹H NMR (400 MHz, CD₃OD) δ 8.54 (s, 1H), 8.41 (dd, J=5.2, 1.4 Hz, 1H), 8.36-8.31 (m, 2H), 8.23 (dd, J=17.6, 1.6 Hz, 1H), 7.96 (ddd, J=8.6, 5.7, 1.2 Hz, 2H), 7.56 (dt, J=4.7, 1.5 Hz, 2H), 7.54-7.51 (m, 1H), 5.39-5.25 (m, 1H), 4.81-4.76 (m, 1H), 4.70-4.62 (m, 2H), 4.37 (d, J=16.7 Hz, 1H), 4.21-4.12 (m, 2H), 3.97 (dd, J=32.2, 12.2 Hz, 1H), 2.70-2.52 (m, 1H), 2.23 (ddd, J=35.0, 14.4, 9.1 Hz, 1H), 1.63 (dd, J=32.3, 7.0 Hz, 3H). LCMS (ESI) (m/z): 584 (M+H)⁺.

### Scheme 332: Synthesis of (2S,4R)—N—((R)-1-(4-carbamimidoylthiophen-2-yl)ethyl)-1-((5,5-dioxidodibenzo[b,d]thiophene-3-carbonyl)glycyl)-4-fluoro-4-(methoxymethyl) pyrrolidine-2-carboxamide (Compound 350)

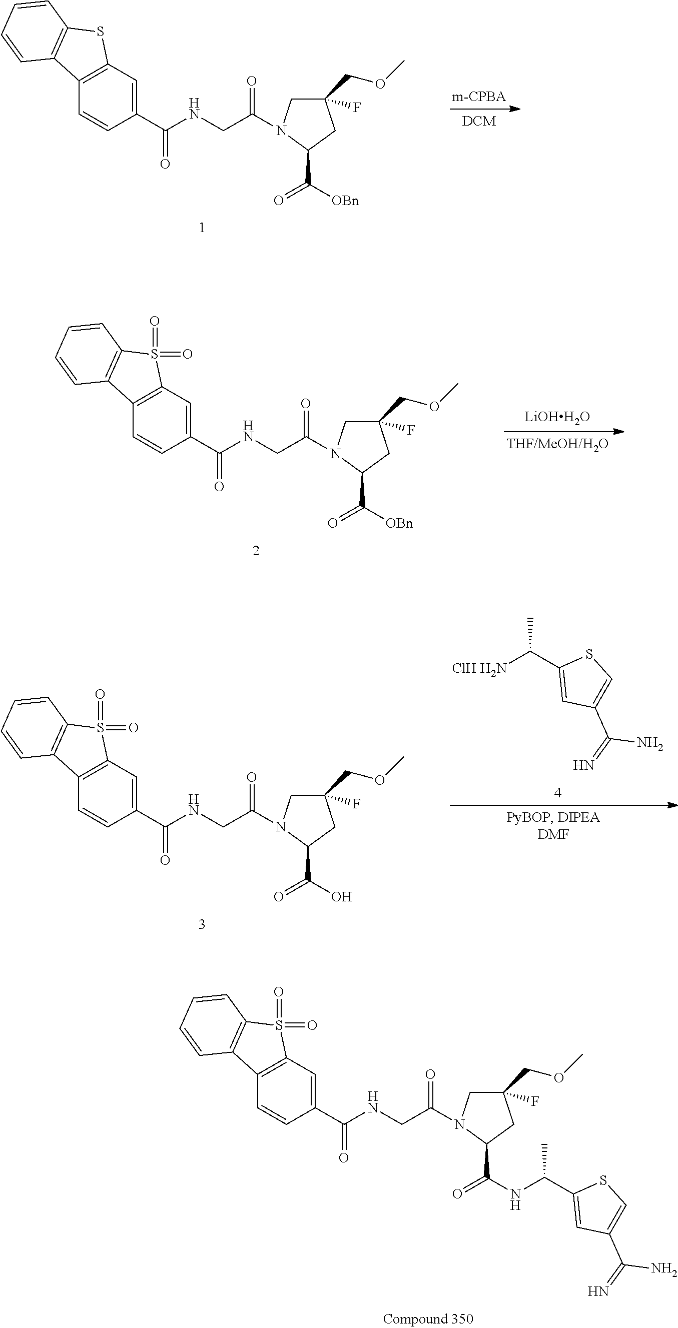

Compound 350 was prepared from benzyl (2S,4R)-1-((dibenzo[b,d]thiophene-3-carbonyl)glycyl)-4-fluoro-4-(methoxymethyl)pyrrolidine-2-carboxylate and (R)-5-(1-aminoethyl)thiophene-3-carboximidamide hydrochloride based on the procedures set forth in Scheme 251. ¹H NMR (400 MHz, CD₃OD) δ 8.53 (s, 1H), 8.30 (dd, J=18.3, 4.0 Hz, 1H), 8.26-8.19 (m, 2H), 8.13 (dd, J=14.4, 7.9 Hz, 2H), 7.89 (d, J=7.7 Hz, 1H), 7.80 (t, J=7.6 Hz, 1H), 7.69 (t, J=7.6 Hz, 1H), 7.55 (d, J=5.7 Hz, 1H), 5.39-5.18 (m, 1H), 4.67-4.57 (m, 1H), 4.18 (ddd, J=38.8, 36.4, 11.3 Hz, 3H), 3.97-3.84 (m, 1H), 3.75-3.65 (m, 2H), 3.43 (d, J=6.6 Hz, 3H), 2.63-2.44 (m, 1H), 2.31-2.10 (m, 1H), 1.61 (dd, J=24.4, 7.1 Hz, 3H). LCMS (ESI) (m/z): 628 (M+H)⁺.

### Scheme 333: Synthesis of (2S,4R)—N—((R)-1-(4-carbamimidoylthiophen-2-yl)ethyl)-4-(cyclopropylsulfonyl)-1-((9,9-difluoro-9H-fluorene-3-carbonyl)glycyl)pyrrolidine-2-carboxamide (Compound 351)

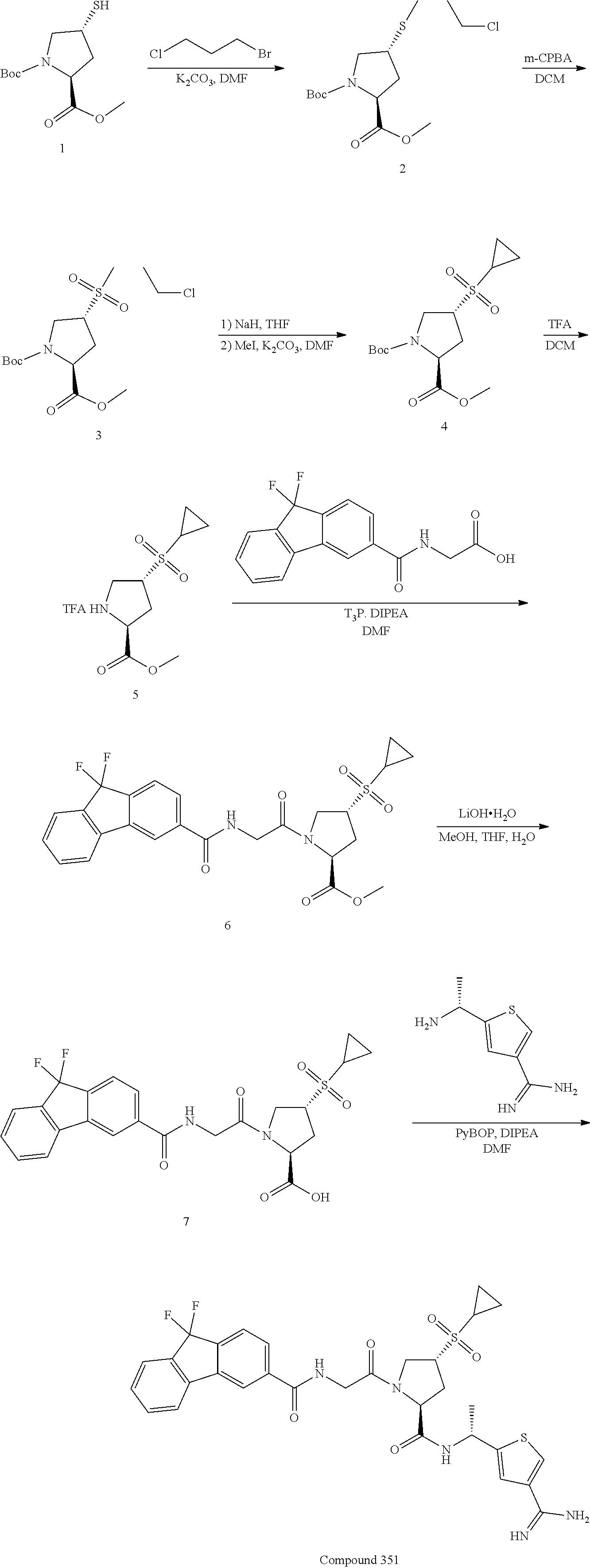

### Step 1: 1-(tert-butyl) 2-methyl (2S,4R)-4-((3-chloropropyl)thio)pyrrolidine-1,2-dicarboxylate

To a solution of 1-(tert-butyl) 2-methyl (2S,4R)-4-mercaptopyrrolidine-1,2-dicarboxylate (1.86 g, 7.13 mmol) and K₂CO₃ (2.95 g, 21.37 mmol) in DMF (20 mL) was added 1-bromo-3-chloropropane (2.21 g, 14.26 mmol) and the mixture was stirred at room temperature overnight. The reaction mixture was diluted with EtOAc, washed with brine, dried over anhydrous Na₂SO₄, filtered, and concentrated under reduced pressure to dryness. The residue was purified by flash chromatography (silica gel, 0-25% EtOAc in PE) to give 1-(tert-butyl) 2-methyl (2S,4R)-4-((3-chloropropyl)thio) pyrrolidine-1,2-dicarboxylate (2.10 g, yield 87.5%) as a light-yellow oil. LC/MS (ESI) m/z: 338 (M+H)⁺.

### Step 2: 1-(tert-butyl) 2-methyl (2S,4R)-4-((3-chloropropyl)sulfonyl)pyrrolidine-1,2-dicarboxylate

To a solution of 1-(tert-butyl) 2-methyl (2S,4R)-4-((3-chloropropyl)thio)pyrrolidine-1,2-dicarboxylate (2.10 g, 6.23 mmol) in DCM (20 mL) was added m-CPBA (2.14 g, 12.46 mmol) at 0° C. and the mixture was stirred at room temperature overnight. The mixture was diluted with water and extracted with DCM twice. The combined organic layers were washed with 5% aq·NaHCO₃ solution and brine, dried over anhydrous Na₂SO₄, filtered, and concentrated under reduced pressure to dryness. The residue was purified by flash chromatography (silica gel, 0-20% EtOAc in PE) to give 1-(tert-butyl) 2-methyl (2S,4R)-4-((3-chloropropyl)sulfonyl)pyrrolidine-1,2-dicarboxylate (1.90 g, yield 82.6%) as a light-yellow oil. LCMS (ESI) (m/z): 370 (M+H)⁺.

### Step 3: 1-(tert-butyl) 2-methyl (2S,4R)-4-(cyclopropylsulfonyl)pyrrolidine-1,2-dicarboxylate

To a solution of 1-(tert-butyl) 2-methyl (2S,4R)-4-((3-chloropropyl)sulfonyl)pyrrolidine-1,2-dicarboxylate (500 mg, 1.36 mmol) in THE (6 mL) was added NaH (163 mg, 4.07 mmol, 60% dispersion in mineral oil) at 0° C. under N₂ atmosphere and the mixture was stirred at 0° C. to room temperature overnight. The reaction mixture was quenched with ice-water and washed with EtOAc. The aqueous layer was acidified with 1 N aq·HCl to pH ˜3 and extracted with EtOAc twice. The combined organic layers were washed with brine, dried over anhydrous Na₂SO₄, filtered, and concentrated under reduced pressure to dryness. The residue was dissolved in DMF (5 mL) and K₂CO₃ (745 mg, 5.40 mmol) and MeI (390 mg, 2.75 mmol) were added. The resulting mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give 1-(tert-butyl) 2-methyl (2S,4R)-4-(cyclopropylsulfonyl)pyrrolidine-1,2-dicarboxylate (330 mg, yield 73.3%) as a light-yellow oil. LC/MS (ESI) m/z: 334 (M+H)⁺.

### Step 4: methyl (2S,4R)-4-(cyclopropylsulfonyl)-1-(2,2,2-trifluoroacetyl)-114-pyrrolidine-2-carboxylate

To a solution of 1-(tert-butyl) 2-methyl (2S,4R)-4-(cyclopropylsulfonyl)pyrrolidine-1,2-dicarboxylate (100 mg, 0.30 mmol) in DCM (2 mL) was added TFA (2 mL) and the reaction mixture was stirred at room temperature for 2 hours. The mixture was concentrated to dryness under reduced pressure to give methyl (2S,4R)-4-(cyclopropylsulfonyl)-1-(2,2,2-trifluoroacetyl)-114-pyrrolidine-2-carboxylate (99 mg, yield 99.9%) as a yellow oil, which was used in next reaction without purification. LC/MS (ESI) m/z: 234 (M+H)⁺.

### Step 5: methyl (2S,4R)-4-(cyclopropylsulfonyl)-1-((9,9-difluoro-9H-fluorene-3-carbonyl)glycyl)pyrrolidine-2-carboxylate

To a mixture of methyl (2S,4R)-4-(cyclopropylsulfonyl)-1-(2,2,2-trifluoroacetyl)-114-pyrrolidine-2-carboxylate (99 mg, 0.30 mmol) and (9,9-difluoro-9H-fluorene-3-carbonyl)glycine (90 mg, 0.30 mmol) in DMF (3 mL) was added DIPEA (155 mg, 1.20 mmol) and T₃P (286 mg, 0.45 mmol, 50% wt. in EtOAc) under N₂ atmosphere and the mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-5% MeOH in DCM) to give methyl (2S,4R)-4-(cyclopropylsulfonyl)-1-((9,9-difluoro-9H-fluorene-3-carbonyl)glycyl) pyrrolidine-2-carboxylate (90 mg, yield 58.1%) as a yellow oil. LC/MS (ESI) m/z: 519 (M+H)⁺.

### Step 6: (2S,4R)-4-(cyclopropylsulfonyl)-1-((9,9-difluoro-9H-fluorene-3-carbonyl) glycyl)pyrrolidine-2-carboxylic acid

To a solution of methyl (2S,4R)-4-(cyclopropylsulfonyl)-1-((9,9-difluoro-9H-fluorene-3-carbonyl)glycyl)pyrrolidine-2-carboxylate (90 mg, 0.17 mmol) in MeOH (2 mL) and THF (1 mL) was added a solution of LiOH·H₂O (29 mg, 0.69 mmol) in water (1 mL) at 0° C. and the mixture was stirred at room temperature for 1 hour. The mixture was diluted with water and washed with EtOAc twice. The aqueous layer was acidified with 1 N aq·HCl to pH˜4 and extracted with EtOAc twice. The combined organic layers were washed with brine, dried over anhydrous Na₂SO₄, filtered, and concentrated to dryness under reduced pressure to give (2S,4R)-4-(cyclopropylsulfonyl)-1-((9,9-difluoro-9H-fluorene-3-carbonyl)glycyl)pyrrolidine-2-carboxylic acid (85 mg, yield 97.7%) as a white solid. LCMS (ESI) (m/z): 505 (M+H)⁺.

### Step 7: (2S,4R)—N—((R)-1-(4-carbamimidoylthiophen-2-yl)ethyl)-4-(cyclopropylsulfonyl)-1-((9,9-difluoro-9H-fluorene-3-carbonyl)glycyl)pyrrolidine-2-carboxamide (Compound 351)

To a mixture of (2S,4R)-4-(cyclopropylsulfonyl)-1-((9,9-difluoro-9H-fluorene-3-carbonyl)glycyl)pyrrolidine-2-carboxylic acid (85 mg, 0.17 mmol) and (R)-5-(1-aminoethyl)thiophene-3-carboximidamide hydrochloride (52 mg, 0.25 mmol) in DMF (3 mL) was added DIPEA (110 mg, 0.85 mmol) and PyBOP (133 mg, 0.26 mmol) at 0° C. under N₂ atmosphere, and the mixture was stirred at room temperature for 1 hour. The reaction mixture was diluted with water and extracted with EtOAc twice. The combined organic layers were washed with brine, dried over anhydrous Na₂SO₄, filtered, and concentrated under reduced pressure to dryness. The residue was purified by prep-HPLC and chiral SFC to give Compound 351 (11 mg, yield 10.0%) as a white solid. ¹H NMR (400 MHz, CD₃OD) δ 8.23-7.99 (m, 2H), 7.87 (t, J=8.3 Hz, 1H), 7.81-7.70 (m, 2H), 7.65 (d, J=7.6 Hz, 1H), 7.57 (t, J=7.6 Hz, 1H), 7.46 (dd, J=16.6, 8.7 Hz, 2H), 5.40-5.23 (m, 1H), 4.72 (dd, J=8.6, 4.5 Hz, 1H), 4.32-4.29 (m, 1H), 4.27-3.92 (m, 4H), 2.85-2.61 (m, 2H), 2.60-2.40 (m, 1H), 1.63 (ddd, J=30.1, 7.0, 2.5 Hz, 3H), 1.21-1.13 (m, 4H). LCMS (ESI) (m/z): 656 (M+H)⁺.

### Scheme 334: Synthesis of (2S,4R)—N—((R)-1-(4-carbamimidoylthiophen-2-yl)ethyl)-1-((dibenzo[b,d]thiophene-3-carbonyl)glycyl)-4-(difluoromethoxy)pyrrolidine-2-carboxamide (Compound 352)

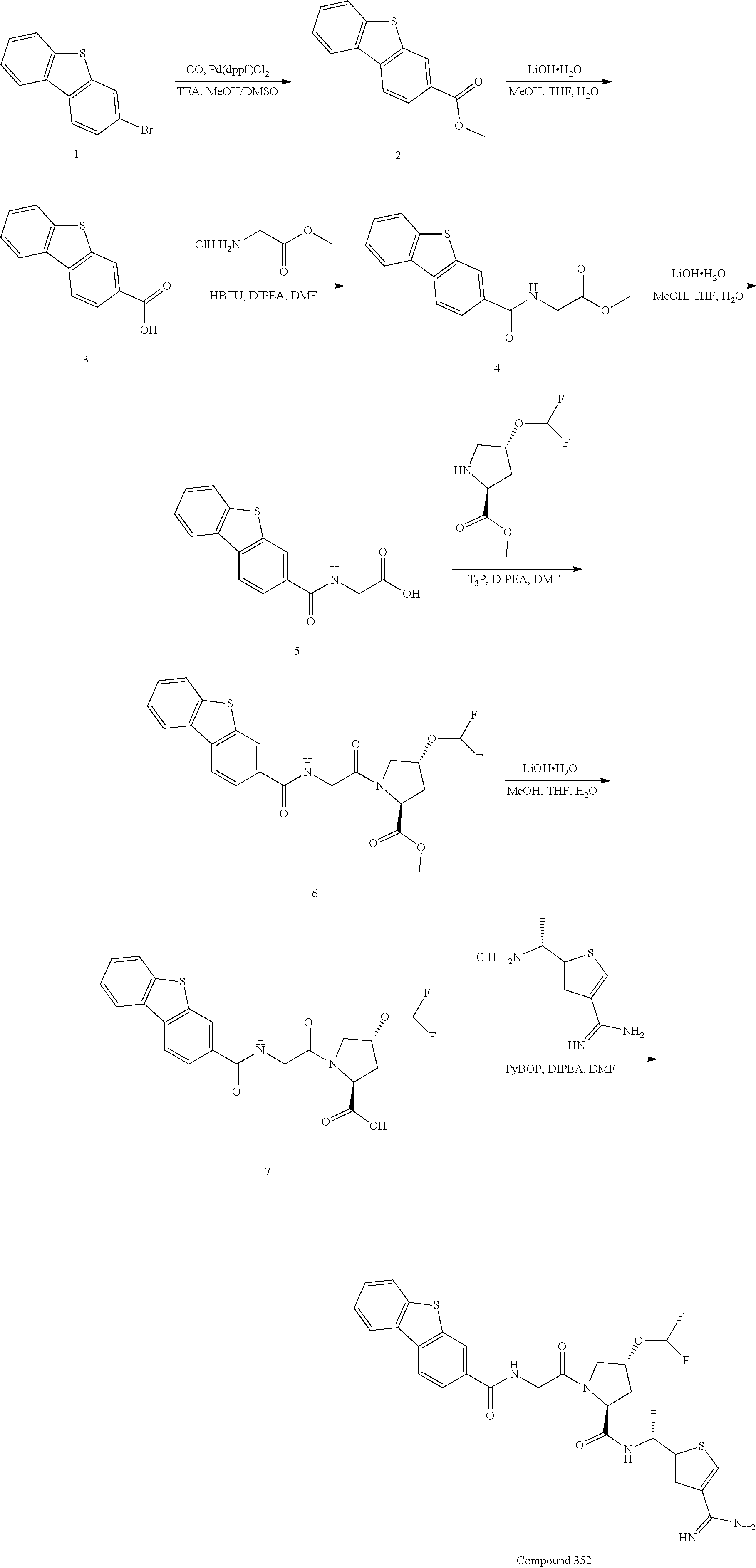

Compound 352 was prepared from 3-bromodibenzo[b,d]thiophene, methyl glycinate hydrochloride, methyl (2S,4R)-4-(difluoromethoxy)pyrrolidine-2-carboxylate, and (R)-5-(1-aminoethyl)thiophene-3-carboximidamide hydrochloride based on the procedures set forth in Scheme 21. ¹H NMR (400 MHz, CD₃OD) δ 8.54 (s, 1H), 8.41 (dd, J=6.0, 1.2 Hz, 1H), 8.34 (dd, J=7.9, 5.9 Hz, 2H), 8.23 (dd, J=14.5, 1.6 Hz, 1H), 7.99-7.94 (m, 2H), 7.58-7.55 (m, 1H), 7.55-7.53 (m, 2H), 6.51 (td, J=74.5, 17.1 Hz, 1H), 5.38-5.25 (m, 1H), 5.04 (s, 1H), 4.60 (t, J=7.9 Hz, 1H), 4.33 (d, J=16.6 Hz, 1H), 4.21 (d, J=16.7 Hz, 1H), 3.98 (dd, J=11.5, 4.5 Hz, 1H), 3.93-3.89 (m, 1H), 2.53-2.46 (m, 1H), 2.28-2.21 (m, 1H), 1.63 (dd, J=31.9, 6.9 Hz, 3H). LCMS (ESI) (m/z): 600 (M+H)⁺.

### Scheme 335: Synthesis of (2S,4R)—N—((R)-1-(4-carbamimidoylthiophen-2-yl)ethyl)-1-((dibenzo[b,d]thiophene-2-carbonyl)glycyl)-4-(difluoromethoxy)pyrrolidine-2-carboxamide (Compound 353)

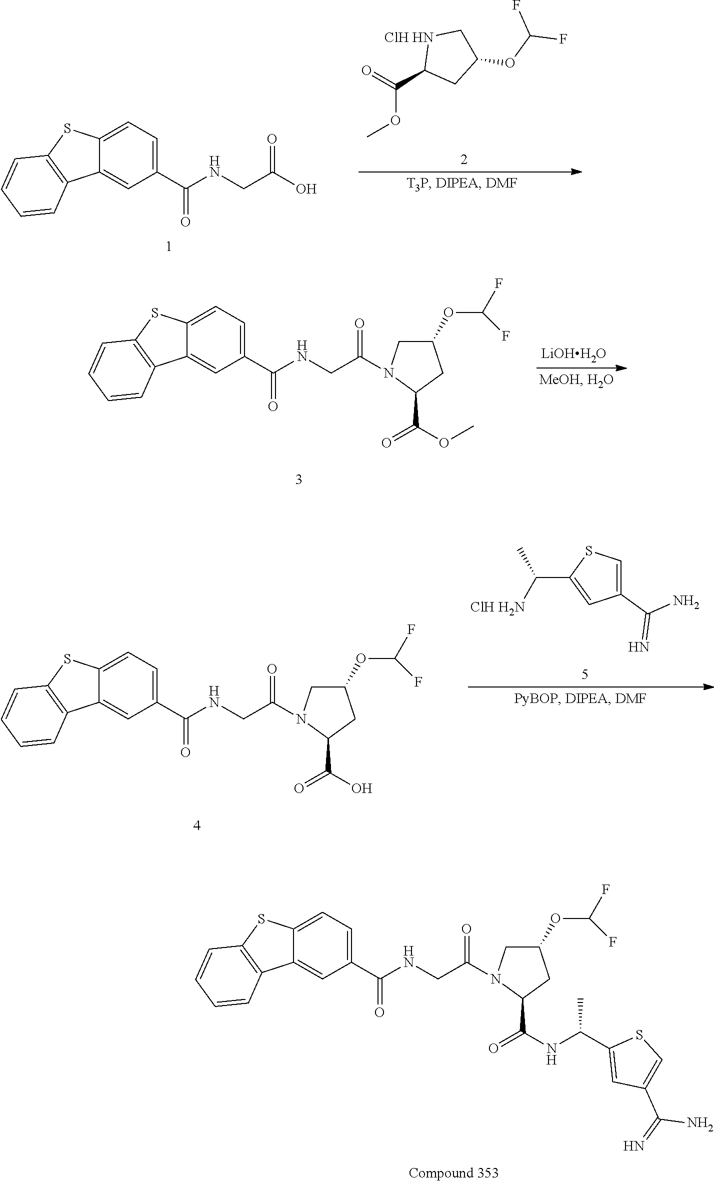

Compound 353 was prepared from (dibenzo[b,d]thiophene-3-carbonyl)glycine, methyl (2S,4R)-4-(difluoromethoxy)pyrrolidine-2-carboxylate hydrochloride, and (R)-5-(1-aminoethyl)thiophene-3-carboximidamide based on the procedures set forth in Scheme 3. ¹H NMR (400 MHz, CD₃OD) δ 8.76 (s, 1H), 8.33-8.24 (m, 1H), 8.17 (t, J=14.5 Hz, 1H), 8.00-7.89 (m, 3H), 7.58-7.49 (m, 3H), 6.52 (td, J=74.5, 18.4 Hz, 1H), 5.35-5.26 (m, 1H), 5.05 (s, 1H), 4.61 (t, J=7.8 Hz, 1H), 4.35 (d, J=16.7 Hz, 1H), 4.25-4.18 (m, 1H), 4.00 (dt, J=10.5, 5.3 Hz, 1H), 3.92 (d, J=11.8 Hz, 1H), 2.54-2.45 (m, 1H), 2.29-2.22 (m, 1H), 1.58 (d, J=7.0 Hz, 3H). LCMS (ESI) (m/z): 600 (M+H)⁺.

### Scheme 336: Synthesis of (2S,4R)—N—((R)-1-(4-carbamimidoylthiophen-2-yl)ethyl)-1-((dibenzo[b,d]thiophene-2-carbonyl)glycyl)-4-fluoro-4-(fluoromethyl)pyrrolidine-2-carboxamide (Compound 354)

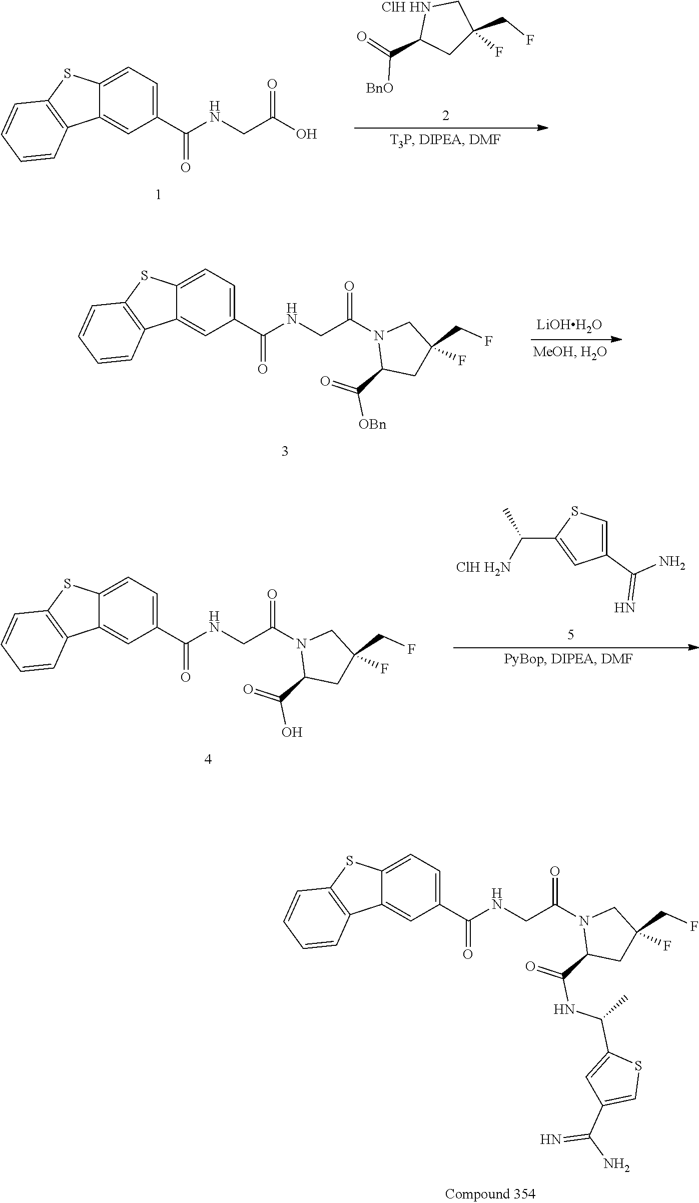

Compound 354 was prepared from (dibenzo[b,d]thiophene-2-carbonyl)glycine, benzyl (2S,4R)-4-fluoro-4-(fluoromethyl)pyrrolidine-2-carboxylate hydrochloride, and (R)-5-(1-aminoethyl)thiophene-3-carboximidamide hydrochloride based on the procedures set forth in Scheme 3. ¹H NMR (400 MHz, CD₃OD) δ 8.73 (s, 1H), 8.55 (s, 1H), 8.24 (dd, J=6.1, 3.0 Hz, 1H), 8.04 (s, 1H), 7.96-7.87 (m, 3H), 7.56-7.50 (m, 3H), 5.34-5.24 (m, 1H), 4.79-4.64 (m, 3H), 4.38 (d, J=16.7 Hz, 1H), 4.16 (dd, J=17.9, 9.4 Hz, 2H), 4.00 (dd, J=32.0, 12.2 Hz, 1H), 2.70-2.55 (m, 1H), 2.30-2.19 (m, 1H), 1.57 (d, J=7.0 Hz, 3H). LCMS (ESI) (m/z): 584 (M+H)⁺.

### Scheme 337: Synthesis of (2S,4R)—N—((R)-1-(4-carbamimidoylthiophen-2-yl)ethyl)-1-((9,9-difluoro-9H-fluorene-3-carbonyl)glycyl)-4-(pyridin-3-yl)pyrrolidine-2-carboxamide (Compound 355)

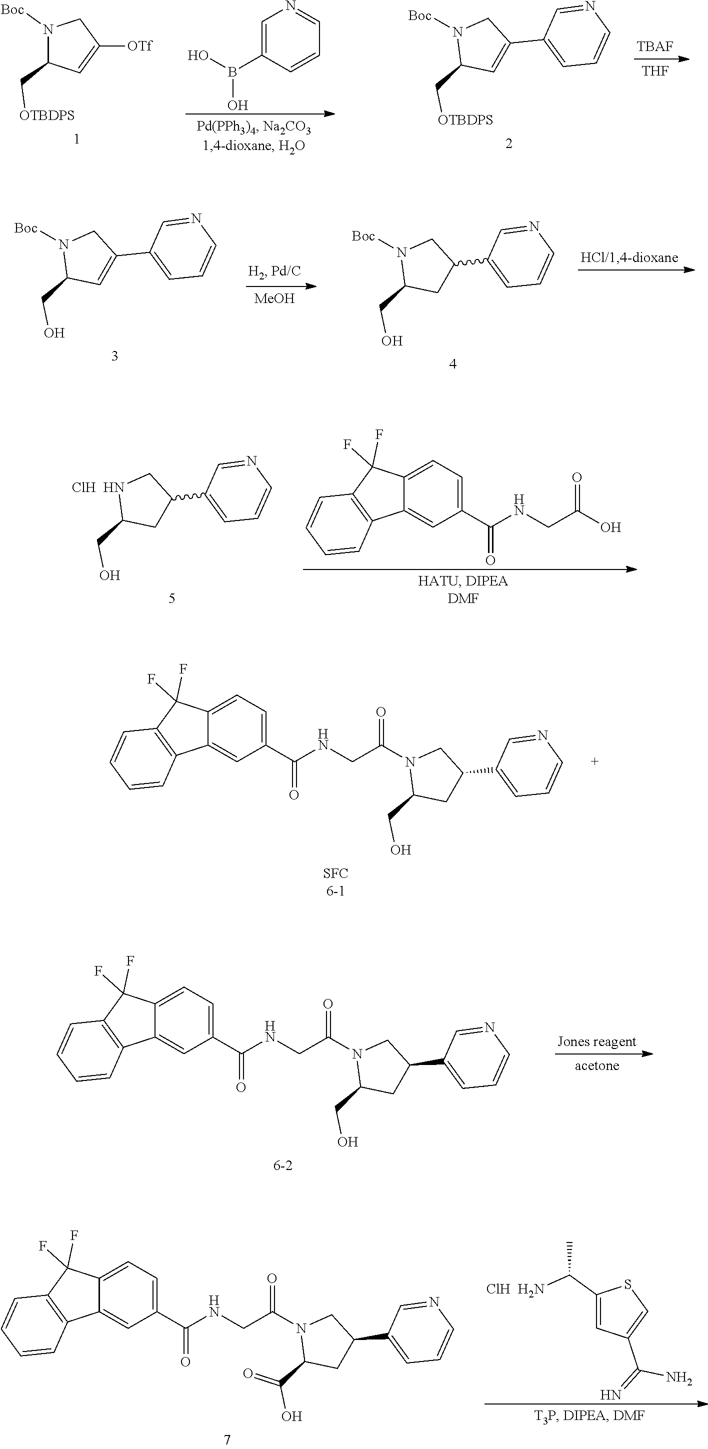

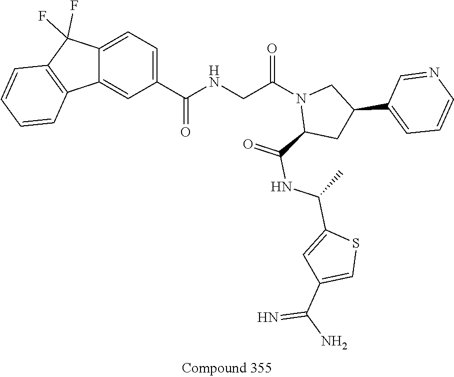 [SMILES: C[C@@H](NC(=O)[C@@H]1C[C@H](c2cccnc2)CN1C(=O)CNC(=O)c1ccc2c(c1)-c1ccccc1C2(F)F)c1cc(C(=N)N)cs1]

### Step 1: tert-butyl (S)-2-(((tert-butyldiphenylsilyl)oxy)methyl)-4-(pyridin-3-yl)-2,5-dihydro-1H-pyrrole-1-carboxylate (2)

To a mixture of tert-butyl (S)-2-(((tert-butyldiphenylsilyl)oxy)methyl)-4-(((trifluoromethyl)sulfonyl)oxy)-2,5-dihydro-1H-pyrrole-1-carboxylate (2.0 g, 3.42 mmol) and pyridin-3-ylboronic acid (841 mg, 6.84 mmol) in 1,4-dioxane (20 mL) and water (7 mL) was added Na₂CO₃ (1.45 g, 13.7 mmol) and Pd(PPh₃)₄ (395 mg, 0.34 mmol) under N₂ atmosphere and the reaction mixture was stirred under N₂ atmosphere at 80° C. for 4 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give tert-butyl (S)-2-(((tert-butyldiphenylsilyl)oxy)methyl)-4-(pyridin-3-yl)-2,5-dihydro-1H-pyrrole-1-carboxylate (1.38 g, yield 78.4%) as a colorless oil. LC/MS (ESI) m/z: 515 (M+H)⁺.

### Step 2: tert-butyl (S)-2-(hydroxymethyl)-4-(pyridin-3-yl)-2,5-dihydro-1H-pyrrole-1-carboxylate (3)

To a solution of tert-butyl (S)-2-(((tert-butyldiphenylsilyl)oxy)methyl)-4-(pyridin-3-yl)-2,5-dihydro-1H-pyrrole-1-carboxylate (1.0 g, 3.62 mmol) in THE (10 mL) was added TBAF (3.6 mL, 3.62 mmol) and the reaction mixture was stirred at room temperature for 2 hours. The mixture was diluted with EtOAc, washed with saturated aq·NH₄Cl solution and brine, dried over anhydrous Na₂SO₄, filtered, and concentrated under reduced pressure to dryness. The residue was purified by flash chromatography (silica gel, 0-10% MeOH in DCM) to give tert-butyl (S)-2-(hydroxymethyl)-4-(pyridin-3-yl)-2,5-dihydro-1H-pyrrole-1-carboxylate (536 mg, yield 53.7%) as a colorless oil. LC/MS (ESI) m/z: 277 (M+H)⁺.

### Step 3: tert-butyl (S)-2-(hydroxymethyl)-4-(pyridin-3-yl)pyrrolidine-1-carboxylate (4)

To a solution of tert-butyl (S)-2-(hydroxymethyl)-4-(pyridin-3-yl)-2,5-dihydro-1H-pyrrole-1-carboxylate (536 mg, 1.94 mmol) in MeOH (10 mL) was added Pd/C (40 mg, 10% wt.), the mixture was degassed under N₂ atmosphere for three times and stirred under a H₂ balloon at 25° C. for 0.5 hour. The mixture was filtered, and the filtrate was concentrated to dryness. The residue was purified by prep-HPLC to give tert-butyl (S)-2-(hydroxymethyl)-4-(pyridin-3-yl)pyrrolidine-1-carboxylate (170 mg, yield 31.5%) as a colorless oil. LC/MS (ESI) m/z: 279 (M+H)⁺.

### Step 4: (S)-(4-(pyridin-3-yl)pyrrolidin-2-yl) methanol hydrochloride (5)

To a solution of tert-butyl (S)-2-(hydroxymethyl)-4-(pyridin-3-yl)pyrrolidine-1-carboxylate (170 mg, 0.61 mmol) in HCl/1,4-dioxane (3 mL, 4 M) and the mixture was stirred at room temperature for 2 hours. The reaction mixture was concentrated under reduced pressure to dryness to give (S)-(4-(pyridin-3-yl)pyrrolidin-2-yl)methanol hydrochloride (130 mg, yield 99.4%) as a colorless oil, which was used directly in the next reaction without further purification. LC/MS (ESI) m/z: 179 (M+H)⁺.

### Step 5: 9,9-difluoro-N-(2-((2S,4S)-2-(hydroxymethyl)-4-(pyridin-3-yl)pyrrolidin-1-yl)-2-oxoethyl)-9H-fluorene-3-carboxamide (6)

To a mixture of (9,9-difluoro-9H-fluorene-3-carbonyl)glycine (156 mg, 0.51 mmol) and (S)-(4-(pyridin-3-yl)pyrrolidin-2-yl)methanol hydrochloride (130 mg, 0.61 mmol) in DMF (3 mL) was added HATU (352 mg, 0.93 mmol) and DIPEA (187 mg, 1.45 mmol) and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) and further purified by SFC to give 9,9-difluoro-N-(2-((2S,4S)-2-(hydroxymethyl)-4-(pyridin-3-yl)pyrrolidin-1-yl)-2-oxoethyl)-9H-fluorene-3-carboxamide (160 mg, yield 55.9%) and 9,9-difluoro-N-(2-((2S,4R)-2-(hydroxymethyl)-4-(pyridin-3-yl)pyrrolidin-1-yl)-2-oxoethyl)-9H-fluorene-3-carboxamide (33 mg, yield 11.5%) as a colorless oil. LC/MS (ESI) m/z: 464 (M+H)⁺.

### Step 6: (2S,4R)-1-((9,9-difluoro-9H-fluorene-3-carbonyl)glycyl)-4-(pyridin-3-yl)pyrrolidine-2-carboxylic acid (7)

To a solution of 9,9-difluoro-N-(2-((2S,4S)-2-(hydroxymethyl)-4-(pyridin-3-yl) pyrrolidin-1-yl)-2-oxoethyl)-9H-fluorene-3-carboxamide (120 mg, 0.26 mmol) in Acetone (3 mL) was added newly prepared Jones reagent (2 mL) at 0° C. and the reaction mixture was stirred at room temperature for 2 hours. The mixture was neutralized with 1 N aq·NaOH to pH ˜6 and purified by reversed flash chromatography (C₁₈, 0-60% MeOH in H₂O with 0.1% FA) to give (2S,4R)-1-((9,9-difluoro-9H-fluorene-3-carbonyl)glycyl)-4-(pyridin-3-yl)pyrrolidine-2-carboxylic acid (30 mg, yield 27.5%) as a yellow oil. LC/MS (ESI) m/z: 478 (M+H)⁺.

### Step 7: (2S,4R)—N—((R)-1-(4-carbamimidoylthiophen-2-yl)ethyl)-1-((9,9-difluoro-9H-fluorene-3-carbonyl)glycyl)-4-(pyridin-3-yl)pyrrolidine-2-carboxamide (Compound 355)

To a mixture of (2S,4R)-1-((9,9-difluoro-9H-fluorene-3-carbonyl)glycyl)-4-(pyridin-3-yl)pyrrolidine-2-carboxylic acid (30 mg, 0.06 mmol) and (R)-5-(1-aminoethyl)thiophene-3-carboximidamide hydrochloridein (14 mg, 0.08 mmol) in DMF (2 mL) was added DIPEA (41 mg, 0.31 mmol) and T₃P (60 mg, 0.09 mmol, 50% wt. in EtOAc) under N₂ atmosphere and the mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=10:1) and further purified by prep-HPLC to give Compound 355 (0.7 mg, yield 1.8%) as a white solid. ¹H NMR (400 MHz, CD₃OD) δ 8.58 (s, 1H), 8.48 (d, J=4.9 Hz, 2H), 8.23-8.20 (m, 2H), 7.92-7.88 (m, 2H), 7.75 (dd, J=14.4, 7.2 Hz, 2H), 7.66 (d, J=6.9 Hz, 1H), 7.60-7.55 (m, 2H), 7.46 (t, J=6.1 Hz, 2H), 5.31 (d, J=5.8 Hz, 1H), 5.20 (s, 1H), 4.59-4.56 (m, 1H), 4.45 (d, J=16.8 Hz, 1H), 4.18 (d, J=16.8 Hz, 1H), 3.76-3.67 (m, 2H), 2.14-2.03 (m, 2H), 1.60 (d, J=7.1 Hz, 3H). LCMS (ESI) (m/z): 629 (M+H)⁺.

### Scheme 338: Synthesis of (2S,4R)—N—((R)-1-(4-carbamimidoylthiophen-2-yl)ethyl)-4-(difluoromethoxy)-1-((9-methyl-9H-fluorene-2-carbonyl)glycyl)pyrrolidine-2-carboxamide (Compound 357)

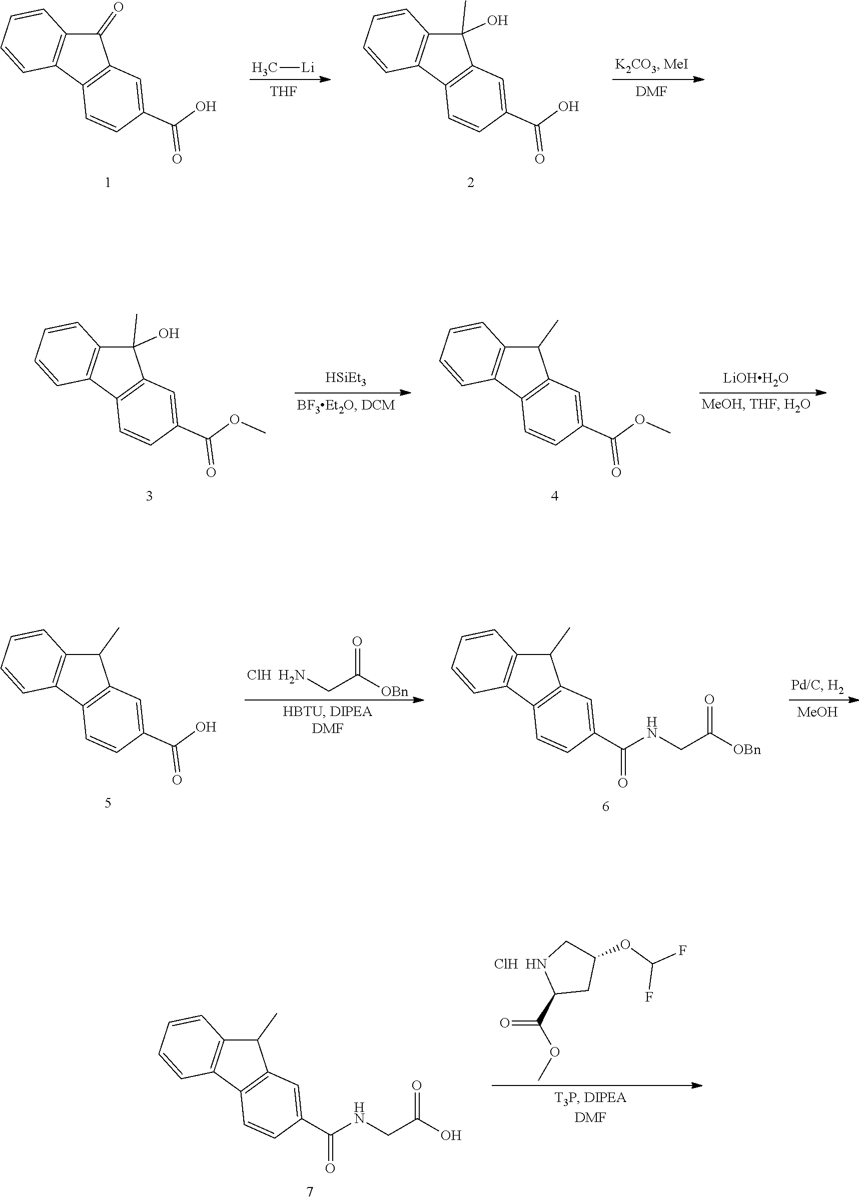

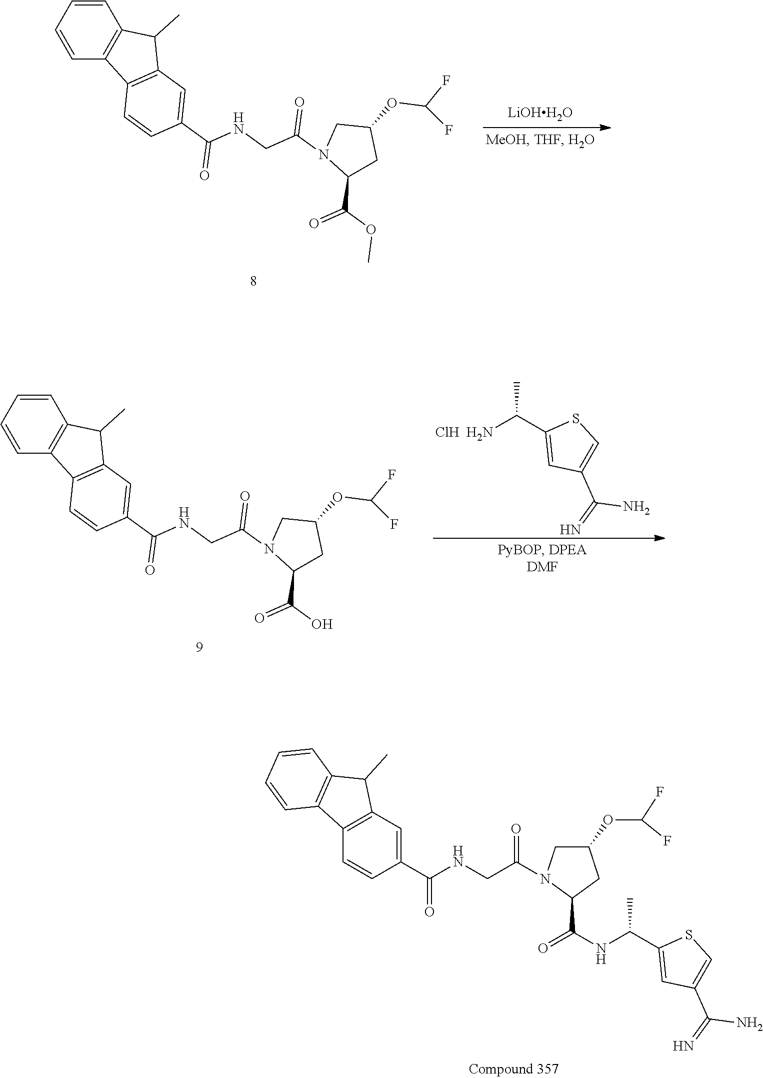

### Step 1: 9-hydroxy-9-methyl-9H-fluorene-2-carboxylic acid (2)

To a solution of 9-oxo-9H-fluorene-2-carboxylic acid (1.0 g, 4.5 mmol) in THE (20 mL) was added methyllithium (10.3 mL, 13.4 mmol) and the reaction mixture was stirred at room temperature for 16 hours. The reaction mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give 9-hydroxy-9-methyl-9H-fluorene-2-carboxylic acid (1.0 g, yield 91.7%) as a white solid. LC/MS (ESI) m/z:241 (M+H)⁺.

### Step 2: methyl 9-hydroxy-9-methyl-9H-fluorene-2-carboxylate (3)

To a solution of 9-hydroxy-9-methyl-9H-fluorene-2-carboxylic acid (1.0 g, 4.2 mmol) in DMF (15 mL) was added K₂CO₃ (2.3 g, 16.7 mmol) and Iodomethane (1.2 g, 8.3 mmol) and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give methyl 9-hydroxy-9-methyl-9H-fluorene-2-carboxylate (860 mg, yield 81.1%) as a white solid. LC/MS (ESI) m/z:255 (M+H)⁺.

### Step 3: methyl 9-methyl-9H-fluorene-2-carboxylate (4)

To a solution of methyl 9-hydroxy-9-methyl-9H-fluorene-2-carboxylate (800 mg, 3.2 mmol) in DCM (15 mL) was added HSiEt₃ (1.8 g, 15.8 mmol) and BF₃·Et₂O (2.2 g, 15.8 mmol) and the reaction mixture was stirred at room temperature for 16 hours. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give methyl 9-methyl-9H-fluorene-2-carboxylate (480 mg, yield 64.0%) as a colorless oil. LC/MS (ESI) m/z:239 (M+H)⁺.

### Step 4: 9-methyl-9H-fluorene-2-carboxylic acid (5)

To a solution of methyl 9-methyl-9H-fluorene-2-carboxylate (480 mg, 2.0 mmol) in MeOH (4 mL), THF (2 mL) and water (2 mL) was added LiOH·H₂O (254 mg, 6.1 mmol) at 0° C. and the mixture was stirred at room temperature for 2 hours. The reaction mixture was acidified with 1 N aq·HCl to pH ˜3 and extracted with EtOAc twice. The combined organic layers were washed with brine, dried over anhydrous Na₂SO₄, filtered, and concentrated under reduced pressure to dryness to give 9-methyl-9H-fluorene-2-carboxylic acid (440 mg, yield 97.4%) as a white solid, which was used in next reaction without purification. LC/MS (ESI) m/z: 225 (M+H)⁺.

### Step 5: benzyl (9-methyl-9H-fluorene-2-carbonyl)glycinate (6)

To a mixture of 9-methyl-9H-fluorene-2-carboxylic acid (440 mg, 2.0 mmol) and benzyl glycinate hydrochloride (594 mg, 3.0 mmol) in DMF (8 mL) was added HBTU (1.1 g, 3.0 mmol) and DIPEA (1.3 g, 9.8 mmol) and the reaction mixture was stirred at room temperature for 16 hours. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give benzyl (9-methyl-9H-fluorene-2-carbonyl)glycinate (659 mg, yield 90.4%) as a colorless oil. LC/MS (ESI) m/z: 372 (M+H)⁺.

### Step 6: (9-methyl-9H-fluorene-2-carbonyl)glycine (7)

To a solution of benzyl (9-methyl-9H-fluorene-2-carbonyl)glycinate (650 mg, 1.8 mmol) in MeOH (8 mL) was added Pd/C (60 mg, 10% wt.), the mixture was degassed under N₂ atmosphere for three times and stirred under a H₂ balloon at 25° C. for 1 hour. The mixture was filtered, and the filtrate was concentrated to dryness to give (9-methyl-9H-fluorene-2-carbonyl)glycine (460 mg, yield 93.5%) as a white solid, which was used in next reaction without purification. LC/MS (ESI) m/z: 282 (M+H)⁺.

### Step 7: methyl (2S,4R)-4-(difluoromethoxy)-1-((9-methyl-9H-fluorene-2-carbonyl) glycyl)pyrrolidine-2-carboxylate (8)

To a mixture of (9-methyl-9H-fluorene-2-carbonyl)glycine (70 mg, 0.25 mmol) and methyl (2S,4R)-4-(difluoromethoxy)pyrrolidine-2-carboxylate hydrochloride (86 mg, 0.37 mmol) in DMF (2 mL) was added DIPEA (160.7 mg, 1.25 mmol) and T₃P (119 mg, 0.37 mmol, 50% wt. in EtOAc) under N₂ atmosphere and the mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-60% EtOAc in PE) to give methyl (2S,4R)-4-(difluoromethoxy)-1-((9-methyl-9H-fluorene-2-carbonyl)glycyl)pyrrolidine-2-carboxylate (85 mg, yield 74.5%) as a yellow oil. LC/MS (ESI) m/z: 459 (M+H)⁺.

### Step 8: (2S,4R)-4-(difluoromethoxy)-1-((9-methyl-9H-fluorene-2-carbonyl)glycyl) pyrrolidine-2-carboxylic acid (9)

To a solution of methyl (2S,4R)-4-(difluoromethoxy)-1-((9-methyl-9H-fluorene-2-carbonyl)glycyl)pyrrolidine-2-carboxylate (85 mg, 0.2 mmol) in MeOH (1 mL), THE (0.5 mL) and water (0.5 mL) was added LiOH·H₂O (24 mg, 0.56 mmol) at 0° C. and the mixture was stirred at room temperature for 2 hours. The reaction mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (2S,4R)-4-(difluoromethoxy)-1-((9-methyl-9H-fluorene-2-carbonyl)glycyl)pyrrolidine-2-carboxylic acid (75 mg, yield 91.0%) as a white solid, which was used in next reaction without purification. LC/MS (ESI) m/z: 445 (M+H)⁺.

### Step 9: (2S,4R)—N—((R)-1-(4-carbamimidoylthiophen-2-yl)ethyl)-4-(difluoromethoxy)-1-((9-methyl-9H-fluorene-2-carbonyl)glycyl)pyrrolidine-2-carboxamide (Compound 357)

To a mixture of (2S,4R)-4-(difluoromethoxy)-1-((9-methyl-9H-fluorene-2-carbonyl) glycyl)pyrrolidine-2-carboxylic acid (50 mg, 0.11 mmol) and (R)-5-(1-aminoethyl)thiophene-3-carboximidamide hydrochloride (30 mg, 0.15 mmol) in DMF (2 mL) was added DIPEA (73 mg, 0.56 mmol) and PyBOP (88 mg, 0.17 mmol) at 0° C. under N₂ atmosphere and the mixture was stirred at room temperature for 1 hour. The reaction mixture was diluted with water and extracted with EtOAc twice. The combined organic layers were washed with brine, dried over anhydrous Na₂SO₄, filtered, and concentrated under reduced pressure to dryness. The residue was purified by prep-TLC (DCM:MeOH=10:1) and further purified by prep-HPLC to give Compound 357 (2.0 mg, yield 3.0%) as a white solid. ¹H NMR (400 MHz, CD₃OD) δ 8.55 (s, 1H), 8.23 (d, J=13.9 Hz, 1H), 8.04 (d, J=10.3 Hz, 1H), 7.90-7.85 (m, 3H), 7.60-7.54 (m, 2H), 7.41-7.37 (m, 2H), 6.52 (dd, J=82.7, 66.1 Hz, 1H), 5.35-5.27 (m, 1H), 5.03 (s, 1H), 4.61 (d, J=7.9 Hz, 1H), 4.31 (d, J=16.7 Hz, 1H), 4.17 (s, 1H), 3.98 (dd, J=16.2, 5.4 Hz, 2H), 3.90 (d, J=11.6 Hz, 1H), 2.52-2.46 (m, 1H), 2.23 (dd, J=7.8, 5.1 Hz, 1H), 1.59 (d, J=7.0 Hz, 3H), 1.55-1.53 (m, 3H). LCMS (ESI) (m/z): 596 (M+H)⁺.

### Scheme 339: Synthesis of (2S,4S)—N—((R)-1-(4-carbamimidoylthiophen-2-yl)ethyl)-1-((9,9-difluoro-9H-fluorene-3-carbonyl)glycyl)-4-(pyridin-2-yl)pyrrolidine-2-carboxamide (Compound 358)

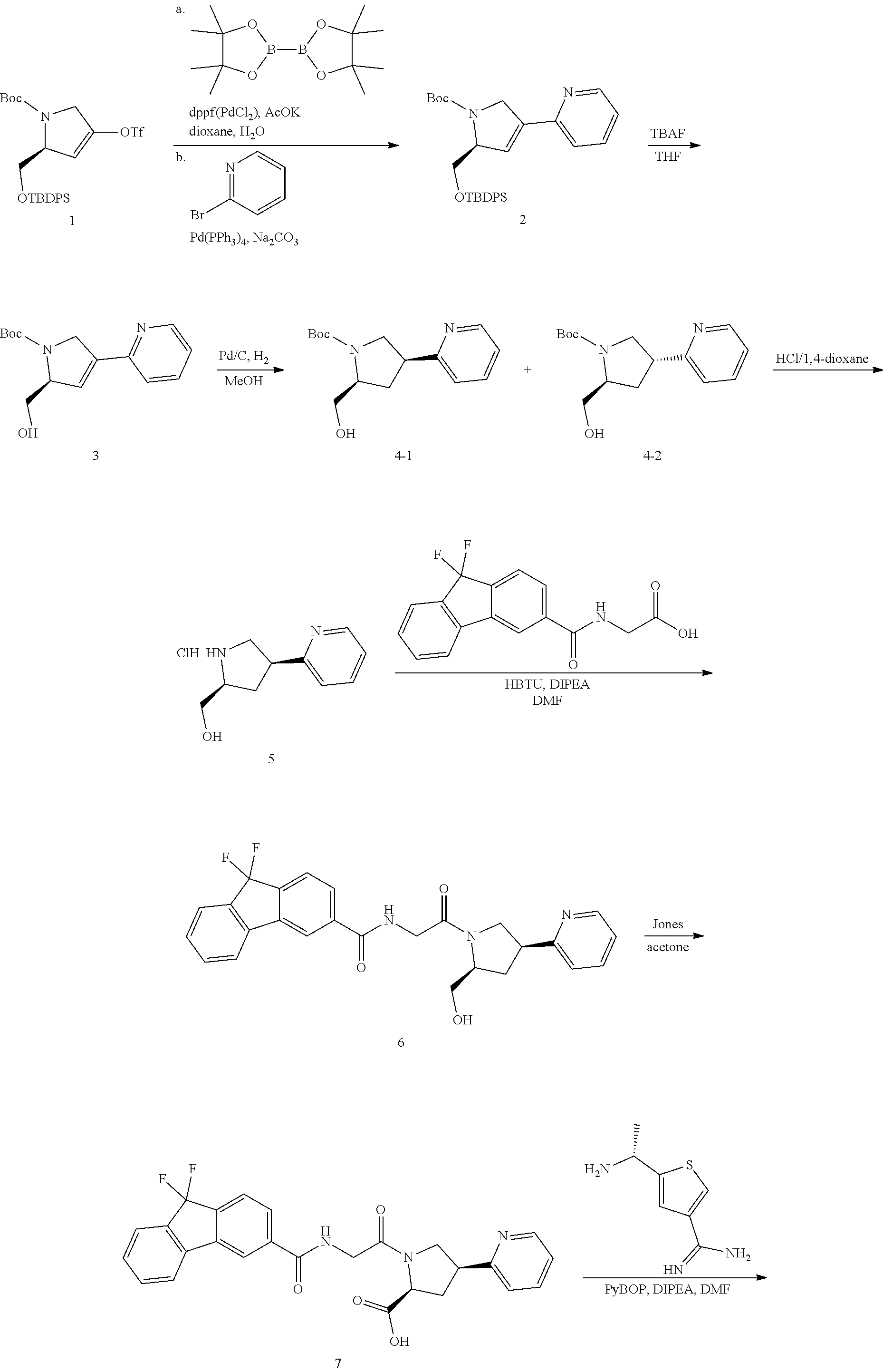

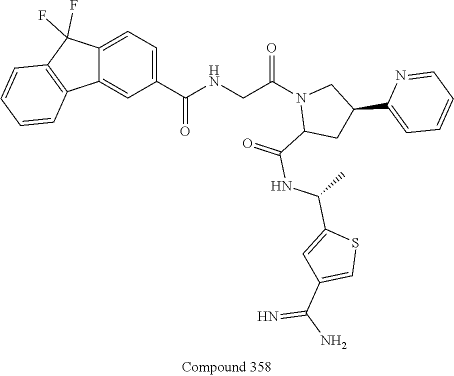 [SMILES: C[C@@H](NC(=O)C1C[C@H](c2ccccn2)CN1C(=O)CNC(=O)c1ccc2c(c1)-c1ccccc1C2(F)F)c1cc(C(=N)N)cs1]

### Step 1: tert-butyl (S)-2-(((tert-butyldiphenylsilyl)oxy)methyl)-4-(pyridin-2-yl)-2,5-dihydro-1H-pyrrole-1-carboxylate (2)

To a mixture of tert-butyl (S)-2-(((tert-butyldiphenylsilyl)oxy)methyl)-4-(((trifluoromethyl)sulfonyl)oxy)-2,5-dihydro-1H-pyrrole-1-carboxylate (2.0 g, 3.42 mmol) and Pin₂B₂ (1.7 g, 6.84 mmol) in 1,4-dioxane (20 mL) and water (6 mL) was added Pd(dppf)Cl₂ (250 mg, 0.34 mmol) and AcOK (1.3 g, 13.6 mmol) under N₂ atmosphere and the reaction mixture was stirred under N₂ atmosphere at 80° C. for 4 hours. The mixture was added Na₂CO₃ (1.1 g, 10.3 mmol), Pd(PPh₃)₄ (197 mg, 0.17 mmol) and 2-bromopyridine (810 mg, 5.13 mmol) under N₂ atmosphere and the reaction mixture was stirred under N₂ atmosphere at 80° C. for 16 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give tert-butyl (S)-2-(((tert-butyldiphenylsilyl) oxy)methyl)-4-(pyridin-2-yl)-2,5-dihydro-1H-pyrrole-1-carboxylate (751 mg, yield 42.7%) as a colorless oil. LC/MS (ESI) m/z: 515 (M+H)⁺.

### Step 2: tert-butyl (S)-2-(hydroxymethyl)-4-(pyridin-2-yl)-2,5-dihydro-1H-pyrrole-1-carboxylate (3)

To a solution of tert-butyl (S)-2-(((tert-butyldiphenylsilyl)oxy)methyl)-4-(pyridin-3-yl)-2,5-dihydro-1H-pyrrole-1-carboxylate (750 mg, 1.5 mmol) in THF (8 mL) was added TBAF (1.5 mL, 1.5 mmol) and the reaction mixture was stirred at room temperature for 2 hours. The mixture was diluted with EtOAc, washed with saturated aq·NH₄Cl solution and brine, dried over anhydrous Na₂SO₄, filtered, and concentrated under reduced pressure to dryness. The residue was purified by flash chromatography (silica gel, 0-10% MeOH in DCM) to give tert-butyl (S)-2-(hydroxymethyl)-4-(pyridin-2-yl)-2,5-dihydro-1H-pyrrole-1-carboxylate (370 mg, yield 92.0%) as a colorless oil. LC/MS (ESI) m/z: 277 (M+H)⁺.

### Step 3: tert-butyl (2S,4S)-2-(hydroxymethyl)-4-(pyridin-2-yl)pyrrolidine-1-carboxylate (4)

To a solution of tert-butyl (S)-2-(hydroxymethyl)-4-(pyridin-2-yl)-2,5-dihydro-1H-pyrrole-1-carboxylate (370 mg, 1.3 mmol) MeOH (8 mL) was added Pd/C (15 mg, 10% wt.), the mixture was degassed under N₂ atmosphere for three times and stirred under a H₂ balloon at 25° C. for 0.5 hour. The mixture was filtered, and the filtrate was concentrated to dryness. The residue was purified by prep-HPLC and purified by prep-SFC to give tert-butyl (2S,4S)-2-(hydroxymethyl)-4-(pyridin-2-yl)pyrrolidine-1-carboxylate (160 mg, yield 42.9%) and tert-butyl (2S,4R)-2-(hydroxymethyl)-4-(pyridin-2-yl)pyrrolidine-1-carboxylate (30 mg, yield 8.0%) as a colorless oil. LC/MS (ESI) m/z: 279 (M+H)⁺.

### Step 4: ((2S,4S)-4-(pyridin-2-yl)pyrrolidin-2-yl)methanol hydrochloride (5)

To a solution of tert-butyl (2S,4S)-2-(hydroxymethyl)-4-(pyridin-2-yl)pyrrolidine-1-carboxylate (150 mg, 0.54 mmol) in HCl/1,4-dioxane (3 mL, 4M) and the mixture was stirred at room temperature for 2 hours. The reaction mixture was concentrated under reduced pressure to dryness to give ((2S,4S)-4-(pyridin-2-yl)pyrrolidin-2-yl)methanol hydrochloride (100 mg, yield 86.5%) as a colorless oil, which was used directly in the next reaction without further purification. LC/MS (ESI) m/z: 179 (M+H)⁺.

### Step 5: 9,9-difluoro-N-(2-((2S,4S)-2-(hydroxymethyl)-4-(pyridin-2-yl)pyrrolidin-1-yl)-2-oxoethyl)-9H-fluorene-3-carboxamide (6)

To a mixture of ((2S,4S)-4-(pyridin-2-yl)pyrrolidin-2-yl)methanol hydrochloride (90 mg, 0.51 mmol) and (9,9-difluoro-9H-fluorene-3-carbonyl)glycine (128 mg, 0.42 mmol) in DMF (3 mL) was added HBTU (240 mg, 0.64 mmol) and DIPEA (272 mg, 2.10 mmol) and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) to give 9,9-difluoro-N-(2-((2S,4S)-2-(hydroxymethyl)-4-(pyridin-2-yl)pyrrolidin-1-yl)-2-oxoethyl)-9H-fluorene-3-carboxamide (140 mg, yield 71.6%) as a colorless oil. LC/MS (ESI) m/z: 464 (M+H)⁺.

### Step 6: (2S,4S)-1-((9,9-difluoro-9H-fluorene-3-carbonyl)glycyl)-4-(pyridin-2-yl) pyrrolidine-2-carboxylic acid (7)

To a solution of 9,9-difluoro-N-(2-((2S,4S)-2-(hydroxymethyl)-4-(pyridin-2-yl)pyrrolidine-1-yl)-2-oxoethyl)-9H-fluorene-3-carboxamide (140 mg, 0.30 mmol) in acetone (4 mL) was added Jones reagent (2 mL) at 0° C. and the reaction mixture was stirred at room temperature for 2 hours. The mixture was neutralized with 1 N aq·NaOH to pH ˜6 and extracted with EtOAc twice. The combined organic layers were washed with brine, dried over anhydrous Na₂SO₄, filtered, and concentrated under reduced pressure to dryness to give (2S,4S)-1-((9,9-difluoro-9H-fluorene-3-carbonyl)glycyl)-4-(pyridin-2-yl)pyrrolidine-2-carboxylic acid (50 mg, yield 34.7%) as a yellow oil. LC/MS (ESI) m/z: 478 (M+H)⁺.

### Step 7: (2S,4S)—N—((R)-1-(4-carbamimidoylthiophen-2-yl)ethyl)-1-((9,9-difluoro-9H-fluorene-3-carbonyl)glycyl)-4-(pyridin-2-yl)pyrrolidine-2-carboxamide (Compound 358)

To a mixture of (2S,4S)-1-((9,9-difluoro-9H-fluorene-3-carbonyl)glycyl)-4-(pyridin-2-yl)pyrrolidine-2-carboxylic acid (45 mg, 0.09 mmol) and (R)-5-(1-aminoethyl) thiophene-3-carboximidamide hydrochloride (25 mg, 0.12 mmol) in DMF (2 mL) was added DIPEA (60.8 mg, 0.47 mmol) and PyBOP (73.6 mg, 0.14 mmol) under N₂ atmosphere and the mixture was stirred at room temperature for 1 hour. The reaction mixture was diluted with water and extracted with EtOAc twice. The combined organic layers were washed with brine, dried over anhydrous Na₂SO₄, filtered, and concentrated under reduced pressure to dryness. The residue was purified by prep-TLC (DCM:MeOH=10:1) and further purified by prep-HPLC to give Compound 358 (1.1 mg, yield 1.9%) as a white solid. ¹H NMR (400 MHz, CD₃OD) δ 8.57-8.51 (m, 2H), 8.22 (d, J=1.6 Hz, 1H), 8.20 (s, 1H), 7.89 (d, J=7.8 Hz, 1H), 7.84-7.80 (m, 1H), 7.79-7.71 (m, 3H), 7.66 (d, J=6.9 Hz, 1H), 7.60 (d, J=6.8 Hz, 1H), 7.55 (s, 1H), 7.45 (s, 2H), 7.33-7.30 (m, 1H), 5.30 (d, J=7.5 Hz, 1H), 4.60-4.56 (m, 2H), 4.42 (d, J=16.9 Hz, 1H), 4.29 (dd, J=9.9, 7.3 Hz, 1H), 4.20 (d, J=16.7 Hz, 1H), 3.91 (d, J=10.6 Hz, 1H), 2.79-2.71 (m, 1H), 2.34-2.26 (m, 1H), 1.63 (d, J=20.9 Hz, 3H). LCMS (ESI) (m/z): 629 (M+H)⁺.

### Scheme 340: Synthesis of (2S,4S)-4-((1H-imidazol-1-yl)methyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (Compound 359)

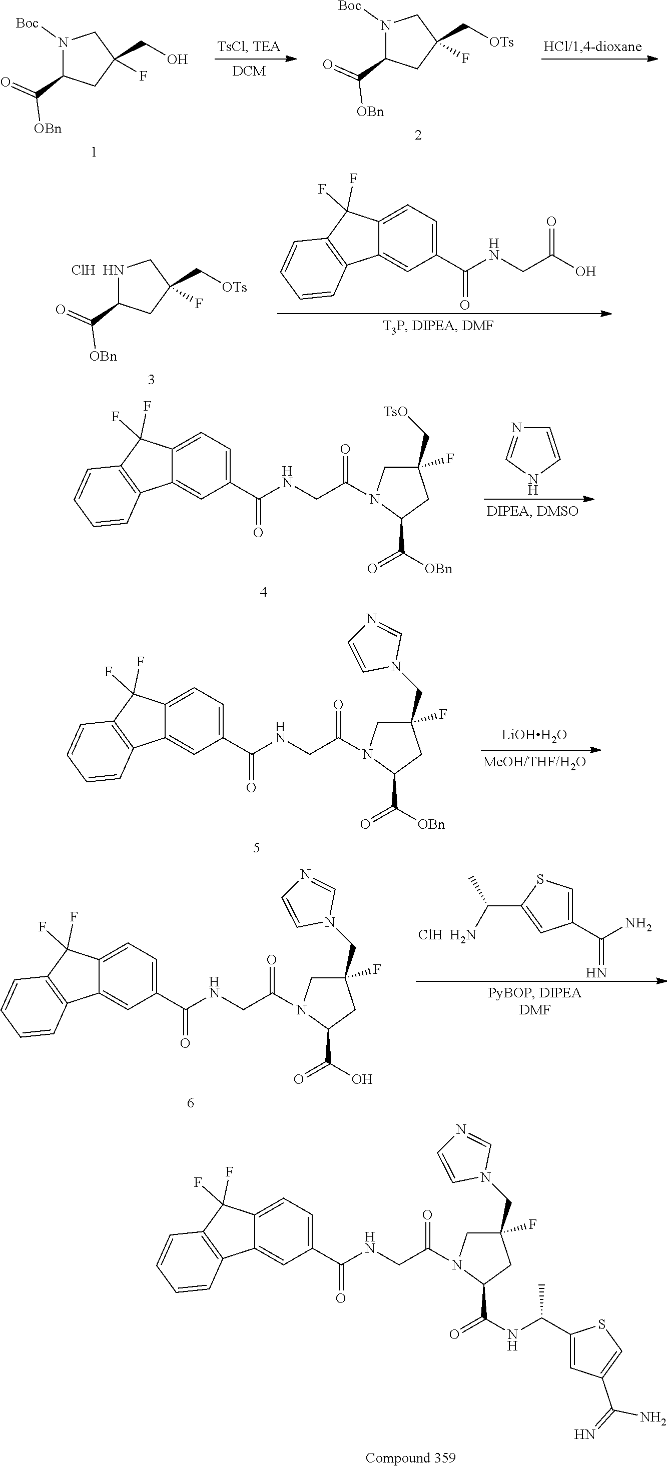

### Step 1: 2-benzyl 1-(tert-butyl) (2S,4R)-4-fluoro-4-((tosyloxy)methyl)pyrrolidine-1,2-dicarboxylate

To a mixture of 2-benzyl 1-(tert-butyl) (2S,4R)-4-fluoro-4-(hydroxymethyl)pyrrolidine-1,2-dicarboxylate (800 mg, 2.27 mmol) and TEA (3.1 mL, 22.66 mmol) in DCM (15 mL) was added TsCl (1.73 g, 9.06 mmol) slowly at 0° C. under N₂ atmosphere and the mixture was stirred at room temperature overnight. The mixture was diluted with DCM and washed with water, dried over anhydrous Na₂SO₄, filtered, and concentrated to dryness. The residue was purified by flash chromatography (silica gel, 0-50% EtOAc in PE) to give 2-benzyl 1-(tert-butyl) (2S,4R)-4-fluoro-4-((tosyloxy)methyl)pyrrolidine-1,2-dicarboxylate (860 mg, yield 74.8%) as a light oil. LC/MS (ESI) m/z: 508 (M+H)⁺.

### Step 2: benzyl (2S,4R)-4-fluoro-4-((tosyloxy)methyl)pyrrolidine-2-carboxylate hydrochloride

To a solution of 2-benzyl 1-(tert-butyl) (2S,4R)-4-fluoro-4-((tosyloxy)methyl) pyrrolidine-1,2-dicarboxylate (700 mg, 1.38 mmol) in DCM (4 mL) was added HCl/1,4-dioxane (6 mL, 4M) and the reaction mixture was stirred at room temperature for 3 hours. The mixture was concentrated to dryness under reduced pressure to give benzyl (2S,4R)-4-fluoro-4-((tosyloxy)methyl)pyrrolidine-2-carboxylate hydrochloride (560 mg, yield 91.7%) as a white solid, which was used in next step without purification. LC/MS (ESI) m/z: 408 (M+H)⁺.

### Step 3: benzyl (2S,4R)-1-((9,9-difluoro-9H-fluorene-3-carbonyl)glycyl)-4-fluoro-4-((tosyloxy)methyl)pyrrolidine-2-carboxylate

To a mixture of benzyl (2S,4R)-4-fluoro-4-((tosyloxy)methyl)pyrrolidine-2-carboxylate hydrochloride (560 mg, 1.26 mmol) and (9,9-difluoro-9H-fluorene-3-carbonyl)glycine (421 mg, 1.39 mmol) in DMF (7 mL) was added DIPEA (813 mg, 6.30 mmol) and T₃P (1.20 g, 1.89 mmol, 50% wt. in EtOAc) and the mixture was stirred at room temperature for 2 hours. The reaction mixture was diluted with EtOAc, washed with saturated aq. NaHCO₃ solution and brine, dried over anhydrous Na₂SO₄, filtered, and concentrated under reduced pressure to dryness. The residue was purified by flash chromatography (silica gel, 0-80% EtOAc in PE) to give benzyl (2S,4R)-1-((9,9-difluoro-9H-fluorene-3-carbonyl)glycyl)-4-fluoro-4-((tosyloxy)methyl)pyrrolidine-2-carboxylate (700 mg, yield 80.1%) as a light oil. LC/MS (ESI) m/z: 693 (M+H)⁺.

### Step 4: benzyl (2S,4S)-4-((1H-imidazol-1-yl)methyl)-1-((9,9-difluoro-9H-fluorene-3-carbonyl)glycyl)-4-fluoropyrrolidine-2-carboxylate

To a solution of benzyl (2S,4R)-1-((9,9-difluoro-9H-fluorene-3-carbonyl)glycyl)-4-fluoro-4-((tosyloxy)methyl)pyrrolidine-2-carboxylate (650 mg, 0.94 mmol) in DMSO (10 mL) was added 1H-imidazole (319 mg, 4.70 mmol) and DIPEA (1.21 g, 9.39 mmol) and the mixture was stirred at 120° C. for 20 hours. The reaction mixture was diluted with EtOAc, washed with brine, dried over anhydrous Na₂SO₄, filtered, and concentrated under reduced pressure to dryness. The residue was purified by flash chromatography (silica gel, 0-2% MeOH in DCM) to give benzyl (2S,4S)-4-((1H-imidazol-1-yl)methyl)-1-((9,9-difluoro-9H-fluorene-3-carbonyl)glycyl)-4-fluoropyrrolidine-2-carboxylate (140 mg, yield 25.4%) as a yellow solid. ¹H NMR (400 MHz, CDCl₃) δ 8.02 (s, 1H), 7.79-7.74 (m, 1H), 7.69-7.61 (m, 3H), 7.54-7.46 (m, 2H), 7.40-7.35 (m, 5H), 7.22 (t, J=3.9 Hz, 1H), 7.10 (d, J=14.7 Hz, 1H), 6.92 (d, J=1.1 Hz, 1H), 5.29-5.18 (m, 2H), 4.76 (t, J=8.1 Hz, 1H), 4.36-4.28 (m, 2H), 4.27-4.13 (m, 2H), 3.86-3.77 (m, 1H), 2.54 (ddd, J=17.4, 14.2, 8.4 Hz, 1H), 2.32-2.26 (m, 1H), 2.11-1.98 (m, 1H). LC/MS (ESI) m/z: 589 (M+H)⁺.

### Step 5: (2S,4S)-4-((1H-imidazol-1-yl)methyl)-1-((9,9-difluoro-9H-fluorene-3-carbonyl) glycyl)-4-fluoropyrrolidine-2-carboxylic acid

To a solution of benzyl (2S,4S)-4-((1H-imidazol-1-yl)methyl)-1-((9,9-difluoro-9H-fluorene-3-carbonyl)glycyl)-4-fluoropyrrolidine-2-carboxylate (60 mg, 0.10 mmol) in MeOH (2 mL) and THF (1 mL) was added a solution of LiOH·H₂O (17 mg, 0.41 mmol) in water (1 mL) at 0° C. and the mixture was stirred at room temperature for 1 hour. The mixture was acidified with 1 N aq·HCl to pH˜4 concentrated to dryness under reduced pressure to give (2S,4S)-4-((1H-imidazol-1-yl)methyl)-1-((9,9-difluoro-9H-fluorene-3-carbonyl)glycyl)-4-fluoropyrrolidine-2-carboxylic acid (46 mg, yield 90.5%) as a yellow solid. LCMS (ESI) (m/z): 499 (M+H)⁺.

### Step 6: (2S,4S)-4-((1H-imidazol-1-yl)methyl)-N—((R)-1-(4-carbamimidoylthiophen-2-yl)ethyl)-1-((9,9-difluoro-9H-fluorene-3-carbonyl)glycyl)-4-fluoropyrrolidine-2-carboxamide (Compound 359)

To a mixture of (2S,4S)-4-((1H-imidazol-1-yl)methyl)-1-((9,9-difluoro-9H-fluorene-3-carbonyl)glycyl)-4-fluoropyrrolidine-2-carboxylic acid (46 mg, 0.092 mmol) and ((R)-5-(1-aminoethyl)thiophene-3-carboximidamide hydrochloride (30 mg, 0.15 mmol) in DMF (3 mL) was added DIPEA (60 mg, 0.47 mmol) and PyBOP (0.14 mg, 72 mmol) at 0° C. under N₂ atmosphere and the mixture was stirred at room temperature for 1 hour. The reaction mixture was diluted with water and extracted with EtOAc twice. The combined organic layers were washed with brine, dried over anhydrous Na₂SO₄, filtered, and concentrated under reduced pressure to dryness. The residue was purified by prep-HPLC to give Compound 359 (4.9 mg, yield 8.2%) as a white solid. ¹H NMR (400 MHz, CD₃OD) δ 8.27-8.09 (m, 2H), 7.89-7.82 (m, 1H), 7.81-7.70 (m, 3H), 7.65 (d, J=7.5 Hz, 1H), 7.61-7.53 (m, 2H), 7.45 (t, J=7.2 Hz, 1H), 7.21 (dd, J=8.5, 1.0 Hz, 1H), 7.04 (d, J=4.9 Hz, 1H), 5.41-5.24 (m, 1H), 4.67-4.52 (m, 3H), 4.20 (dt, J=34.1, 17.0 Hz, 2H), 4.05-3.86 (m, 2H), 2.59-2.37 (m, 1H), 2.22 (ddd, J=35.6, 13.9, 9.3 Hz, 1H), 1.62 (dd, J=33.5, 7.0 Hz, 3H). LCMS (ESI) (m/z): 650 (M+H)⁺.

### Scheme 341: Synthesis of (2S,4R)—N—((R)-1-(4-carbamimidoylthiophen-2-yl)ethyl)-1-((dibenzo[b,d]furan-3-carbonyl)glycyl)-4-fluoro-4-(methoxymethyl)pyrrolidine-2-carboxamide (Compound 360)

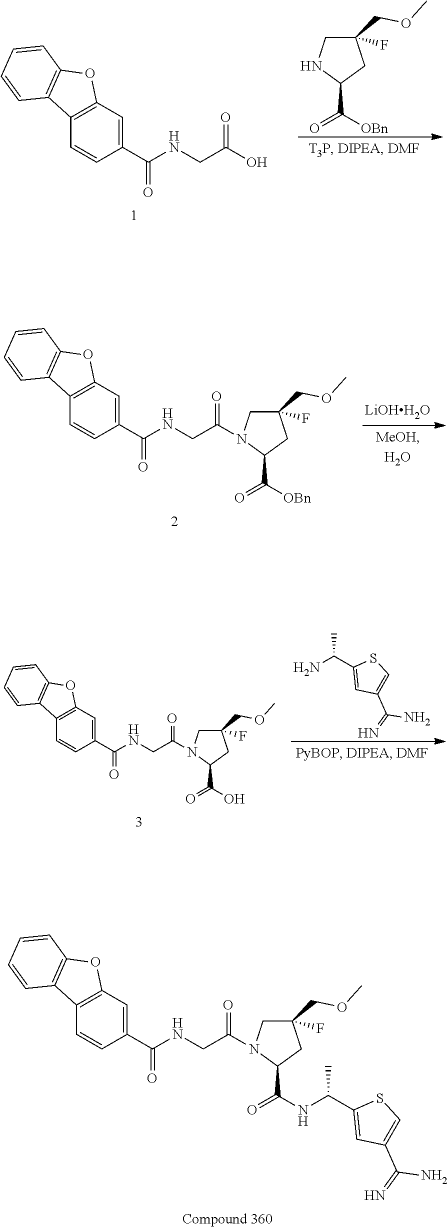

Compound 360 was prepared from (dibenzo[b,d]furan-3-carbonyl)glycine, benzyl (2S,4R)-4-fluoro-4-(methoxymethyl)pyrrolidine-2-carboxylate, and (R)-5-(1-aminoethyl)thiophene-3-carboximidamide based on the procedures set forth in Scheme 3. ¹H NMR (400 MHz, CD₃OD) δ 8.55 (s, 1H), 8.20 (d, J=1.6 Hz, 1H), 8.10 (dd, J=11.1, 4.4 Hz, 3H), 7.89 (dd, J=8.1, 1.5 Hz, 1H), 7.64 (d, J=8.3 Hz, 1H), 7.56 (ddd, J=5.3, 4.7, 1.6 Hz, 2H), 7.43 (dd, J=10.9, 4.1 Hz, 1H), 5.36-5.24 (m, 1H), 4.68-4.61 (m, 1H), 4.35 (d, J=16.7 Hz, 1H), 4.19-4.04 (m, 2H), 3.98-3.86 (m, 1H), 3.73 (d, J=4.5 Hz, 1H), 3.69 (d, J=7.0 Hz, 1H), 3.43 (d, J=5.3 Hz, 3H), 2.63-2.49 (m, 1H), 2.20 (ddd, J=36.8, 14.2, 9.5 Hz, 1H), 1.58 (d, J=7.0 Hz, 3H). LCMS (ESI) (m/z): 580 (M+H)⁺.

### Scheme 342: Synthesis of (2S,4R)—N—((R)-1-(4-carbamimidoylthiophen-2-yl)ethyl)-1-((dibenzo[b,d]furan-3-carbonyl)glycyl)-4-(difluoromethoxy)pyrrolidine-2-carboxamide (Compound 361)

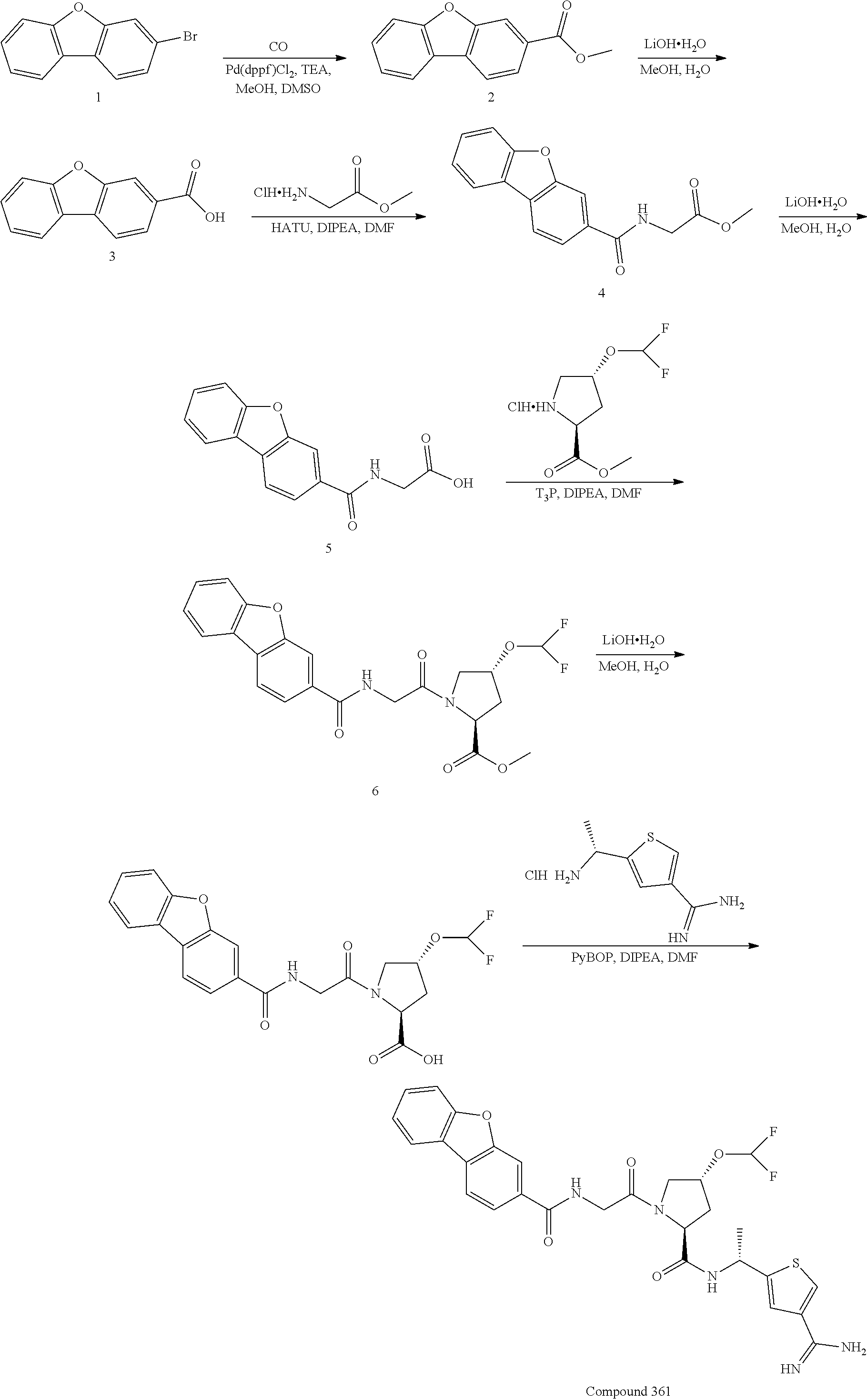

Compound 361 was prepared from 3-bromodibenzo[b,d]furan, methyl glycinate hydrochloride, methyl (2S,4R)-4-(difluoromethoxy)pyrrolidine-2-carboxylate hydrochloride, and (R)-5-(1-aminoethyl)thiophene-3-carboximidamide based on the procedures set forth in Scheme 21. ¹H NMR (400 MHz, CD₃OD) δ 8.52 (s, 1H), 8.23 (dd, J=16.8, 1.6 Hz, 1H), 8.14-8.08 (m, 3H), 7.88 (td, J=8.4, 1.5 Hz, 1H), 7.64 (d, J=8.3 Hz, 1H), 7.56 (ddd, J=5.0, 4.5, 1.3 Hz, 2H), 7.44-7.40 (m, 1H), 6.51 (td, J=74.4, 17.2 Hz, 1H), 5.27 (q, J=6.8 Hz, 1H), 5.04 (m, 1H), 4.60 (t, J=7.8 Hz, 1H), 4.26 (dd, J=48.9, 16.7 Hz, 2H), 3.95 (ddd, J=21.8, 11.6, 4.9 Hz, 2H), 2.66-2.45 (m, 1H), 2.40-2.21 (m, 1H), 1.59 (d, J=7.0 Hz, 3H). LCMS (ESI) (m/z): 584 (M+H)⁺.

### Scheme 343: Synthesis of (2S,4R)—N—((R)-1-(4-carbamimidoylthiophen-2-yl)ethyl)-4-(difluoromethoxy)-1-((5,5-dioxidodibenzo[b,d]thiophene-3-carbonyl)glycyl) pyrrolidine-2-carboxamide (Compound 362)

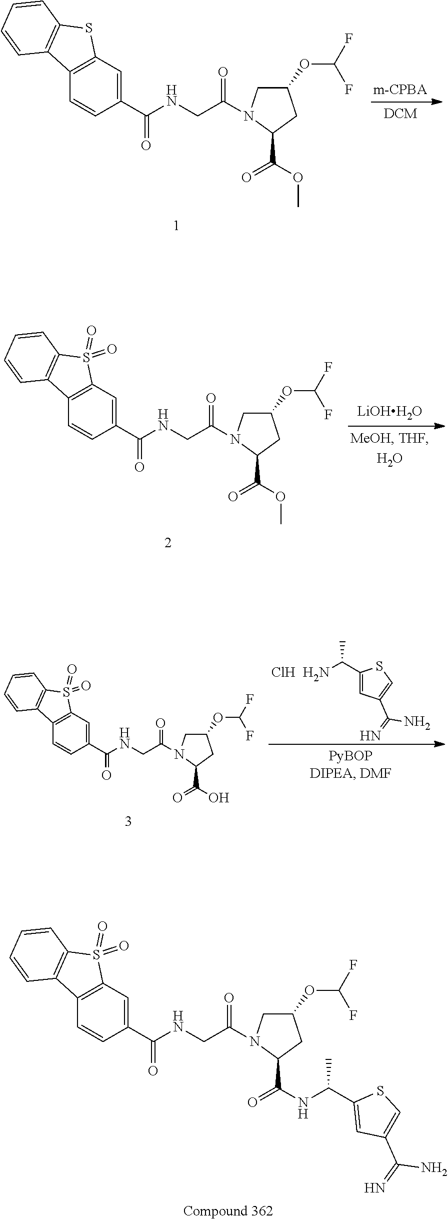

Compound 362 was prepared from methyl (2S,4R)-1-((dibenzo[b,d]thiophene-3-carbonyl)glycyl)-4-(difluoromethoxy)pyrrolidine-2-carboxylate and (R)-5-(1-aminoethyl)thiophene-3-carboximidamide hydrochloride based on the procedures set forth in Scheme 251. ¹H NMR (400 MHz, CD₃OD) δ 8.49 (s, 1H), 8.31 (dd, J=13.9, 1.3 Hz, 1H), 8.25-8.20 (m, 2H), 8.14 (dd, J=14.6, 7.9 Hz, 2H), 7.90 (d, J=7.7 Hz, 1H), 7.80 (td, J=7.6, 1.0 Hz, 1H), 7.69 (t, J=7.6 Hz, 1H), 7.54 (dd, J=5.5, 4.2 Hz, 1H), 6.51 (td, J=74.4, 17.3 Hz, 1H), 5.37-5.24 (m, 1H), 5.07-5.00 (m, 1H), 4.62 (dt, J=15.7, 7.7 Hz, 1H), 4.21 (dt, J=24.3, 16.7 Hz, 2H), 3.97 (dd, J=11.4, 4.4 Hz, 1H), 3.90 (t, J=9.8 Hz, 1H), 2.47 (ddd, J=28.6, 16.5, 7.2 Hz, 1H), 2.33-2.15 (m, 1H), 1.73-1.58 (m, 3H). LCMS (ESI) (m/z): 632 (M+H)⁺.

### Scheme 344: Synthesis of (2S,4R)—N—((R)-1-(4-carbamimidoylthiophen-2-yl)ethyl)-4-fluoro-4-(methoxymethyl)-1-((9-methyl-9H-fluorene-2-carbonyl)glycyl)pyrrolidine-2-carboxamide (Compound 363)

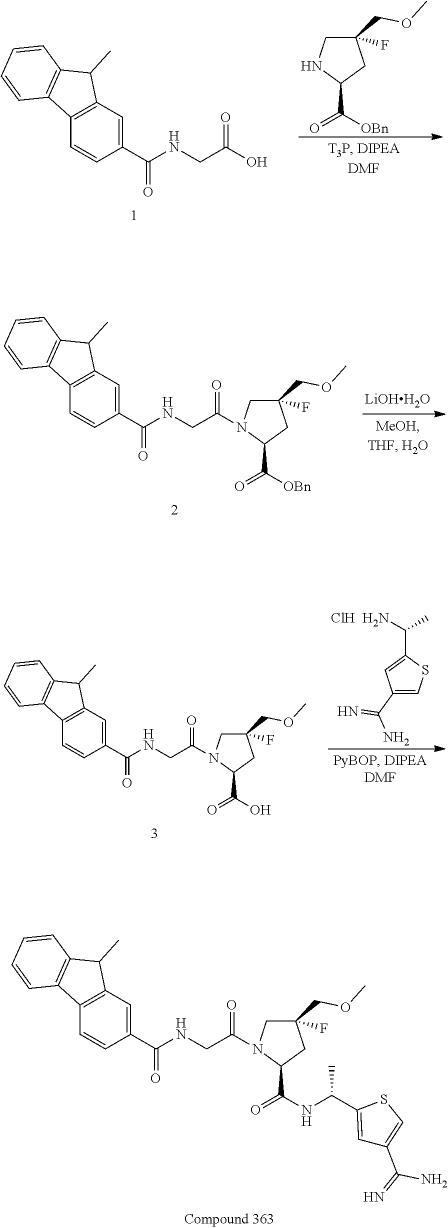

Compound 363 was prepared from (9-methyl-9H-fluorene-2-carbonyl)glycine, benzyl (2S,4R)-4-fluoro-4-(methoxymethyl)pyrrolidine-2-carboxylate hydrochloride, and (R)-5-(1-aminoethyl)thiophene-3-carboximidamide hydrochloride based on the procedures set forth in Scheme 3. ¹H NMR (400 MHz, CD₃OD) δ 8.53 (s, 1H), 8.26-8.20 (m, 1H), 8.06-8.03 (m, 1H), 7.89-7.84 (m, 3H), 7.60-7.55 (m, 2H), 7.41-7.37 (m, 2H), 5.36-5.24 (m, 1H), 4.63 (dd, J=9.2, 8.1 Hz, 1H), 4.40-4.25 (m, 1H), 4.19-4.06 (m, 2H), 3.95 (ddd, J=21.4, 8.2, 7.4 Hz, 2H), 3.75-3.67 (m, 2H), 3.43 (d, J=5.0 Hz, 3H), 2.64-2.46 (m, 1H), 2.20 (ddd, J=37.1, 14.2, 9.6 Hz, 1H), 1.60-1.52 (m, 6H). LC/MS (ESI) (m/z): 592(M+H)⁺.

### Scheme 345: Synthesis of (2S,4R)—N—((R)-1-(4-carbamimidoylthiophen-2-yl)ethyl)-4-fluoro-1-((4′-fluoro-[1,1′-biphenyl]-3-carbonyl)glycyl)-4-(methoxymethyl)pyrrolidine-2-carboxamide (Compound 364)

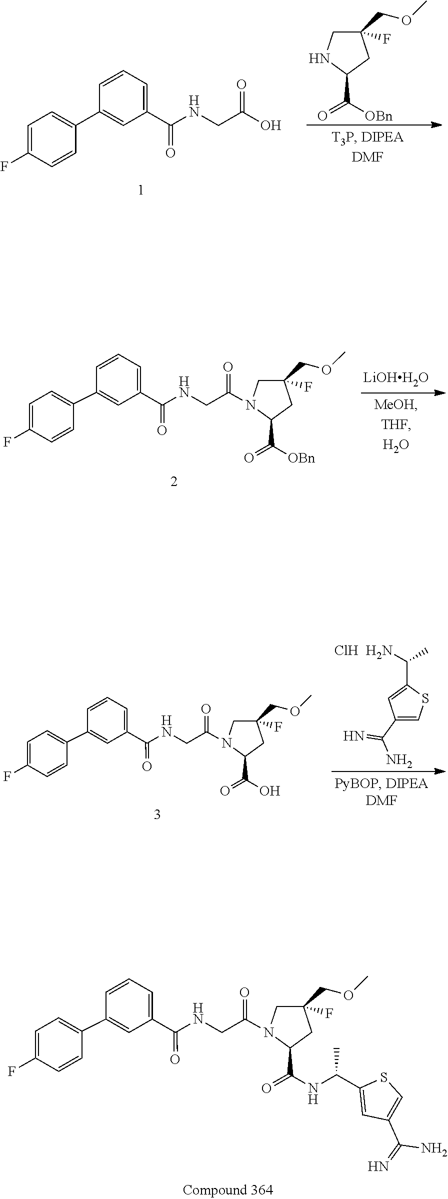

Compound 364 was prepared from methyl (9,9-dimethyl-9H-fluorene-2-carbonyl)glycine, benzyl (2S,4R)-4-fluoro-4-(methoxymethyl)pyrrolidine-2-carboxylate hydrochloride, and (R)-5-(1-aminoethyl)thiophene-3-carboximidamide hydrochloride based on the procedures set forth in Scheme 3. ¹H NMR (400 MHz, CD₃OD) δ 8.55 (s, 1H), 8.25-8.07 (m, 2H), 7.84-7.78 (m, 2H), 7.69 (ddd, J=5.3, 4.6, 2.0 Hz, 2H), 7.58-7.52 (m, 2H), 7.20 (t, J=8.7 Hz, 2H), 5.38-5.23 (m, 1H), 4.66-4.58 (m, 1H), 4.33 (d, J=16.7 Hz, 1H), 4.18-4.12 (m, 1H), 4.07 (ddd, J=13.7, 12.4, 4.0 Hz, 1H), 3.97-3.85 (m, 1H), 3.74-3.66 (m, 2H), 3.43 (d, J=4.5 Hz, 3H), 2.61-2.48 (m, 1H), 2.19 (ddd, J=36.8, 14.2, 9.5 Hz, 1H), 1.58 (d, J=7.0 Hz, 3H). LC/MS (ESI) (m/z): 584 (M+H)⁺.

### Scheme 346: Synthesis of (1S,3S,5R)—N—((R)-1-(4-carbamimidoylthiophen-2-yl)ethyl)-2-((9,9-difluoro-9H-fluorene-3-carbonyl)glycyl)-5-((2-(dimethylamino)ethoxy)methyl)-2-azabicyclo[3.1.0]hexane-3-carboxamide (Compound 367)

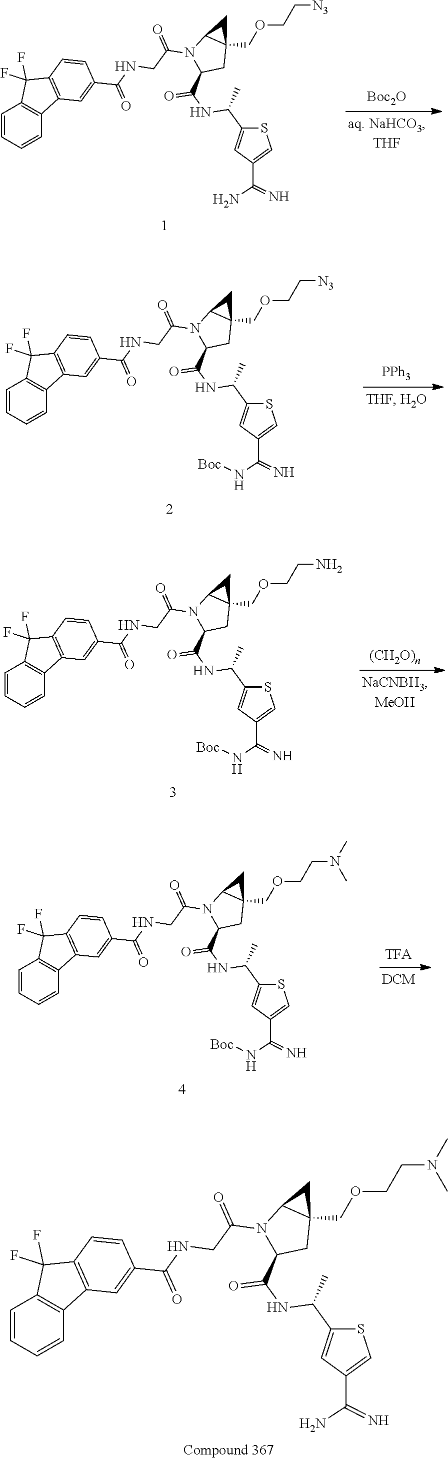

Step 1: tert-butyl ((5-((R)-1-((1S,3S,5R)-5-((2-azidoethoxy)methyl)-2-((9,9-difluoro-9H-fluorene-3-carbonyl)glycyl)-2-azabicyclo[3.1.0]hexane-3-carboxamido)ethyl) thiophen-3-yl)(imino)methyl)carbamate

To a mixture of (1S,3S,5R)-5-((2-azidoethoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (108 mg, 0.16 mmol) and NaHCO₃ (137 mg, 1.60 mmol) in THF (2 mL) and H₂O (2 mL) was added Boc₂O (52 mg, 0.24 mmol) and the reaction mixture was stirred at room temperature for 16 hours. The mixture was diluted with EtOAc, washed with water and brine, dried over anhydrous Na₂SO₄, filtered, and concentrated under reduced pressure to dryness. The residue was purified by flash chromatography (silica gel, 0-7% MeOH in DCM) to give tert-butyl ((5-((R)-1-((1 S,3S,5R)-5-((2-azidoethoxy)methyl)-2-((9,9-difluoro-9H-fluorene-3-carbonyl)glycyl)-2-azabicyclo[3.1.0]hexane-3-carboxamido)ethyl)thiophen-3-yl)(imino)methyl) carbamate (65 mg, yield 52.4%) as a white solid. LC/MS (ESI) m/z: 763 (M+H)⁺.

### Step 2: tert-butyl ((5-((R)-1-((1S,3S,5R)-5-((2-aminoethoxy)methyl)-2-((9,9-difluoro-9H-fluorene-3-carbonyl)glycyl)-2-azabicyclo[3.1.0]hexane-3-carboxamido)ethyl) thiophen-3-yl)(imino)methyl)carbamate

To a solution of tert-butyl ((5-((R)-1-((1 S,3S,5R)-5-((2-azidoethoxy)methyl)-2-((9,9-difluoro-9H-fluorene-3-carbonyl)glycyl)-2-azabicyclo[3.1.0]hexane-3-carboxamido) ethyl)thiophen-3-yl)(imino)methyl)carbamate (65 mg, 0.08 mmol) in THE (5 mL) and H₂O (0.5 mL) was added PPh₃ (45 mg, 0.16 mmol), the mixture was degassed under N₂ atmosphere for five times and stirred at 45° C. for 16 hours. The reaction mixture was concentrated under reduced pressure to dryness. The residue was purified by prep-TLC (DCM:MeOH=7:1) to give tert-butyl ((5-((R)-1-((1S,3S,5R)-5-((2-aminoethoxy) methyl)-2-((9,9-difluoro-9H-fluorene-3-carbonyl)glycyl)-2-azabicyclo [3.1.0]hexane-3-carboxamido)ethyl)thiophen-3-yl)(imino)methyl)carbamate (30 mg, yield 47.6%) as a white solid. LC/MS (ESI) m/z: 737 (M+H)⁺.

### Step 3: tert-butyl ((5-((R)-1-((1S,3S,5R)-2-((9,9-difluoro-9H-fluorene-3-carbonyl) glycyl)-5-((2-(dimethylamino)ethoxy)methyl)-2-azabicyclo[3.1.0]hexane-3-carboxamido)ethyl)thiophen-3-yl)(imino)methyl)carbamate

To a mixture of tert-butyl ((5-((R)-1-((1S,3S,5R)-5-((2-aminoethoxy)methyl)-2-((9,9-difluoro-9H-fluorene-3-carbonyl)glycyl)-2-azabicyclo[3.1.0]hexane-3-carboxamido) ethyl)thiophen-3-yl)(imino)methyl)carbamate (45 mg, 0.06 mmol) and (CH₂O)ₙ (5.3 mg, 0.30 mmol) in MeOH (3 mL) was added NaBH₃CN (23 mg, 0.36 mmol) at 0° C. and the mixture was stirred at room temperature for 16 hours. The mixture was quenched with ice-water and extracted with EtOAc, washed with brine, dried over anhydrous Na₂SO₄, filtered, and concentrated under reduced pressure to give tert-butyl ((5-((R)-1-((1 S,3S,5R)-2-((9,9-difluoro-9H-fluorene-3-carbonyl)glycyl)-5-((2-(dimethylamino) ethoxy)methyl)-2-azabicyclo[3.1.0]hexane-3-carboxamido)ethyl)thiophen-3-yl)(imino) methyl)carbamate (45 mg, yield 95.7%) as a white solid. LC/MS (ESI) m/z: 765 (M+H)⁺.

### Step 4: (1S,3S,5R)—N—((R)-1-(4-carbamimidoylthiophen-2-yl)ethyl)-2-((9,9-difluoro-9H-fluorene-3-carbonyl)glycyl)-5-((2-(dimethylamino)ethoxy)methyl)-2-azabicyclo [3.1.0]hexane-3-carboxamide (Compound 367)

To a solution of tert-butyl ((5-((R)-1-((1 S,3S,5R)-2-((9,9-difluoro-9H-fluorene-3-carbonyl)glycyl)-5-((2-(dimethylamino)ethoxy)methyl)-2-azabicyclo[3.1.0]hexane-3-carboxamido)ethyl)thiophen-3-yl)(imino)methyl)carbamate (45 mg, 0.06 mmol) in DCM (2 mL) was added TFA (1 mL) at 0° C. and the mixture was stirred at room temperature for 1 hour. The mixture was concentrated under reduced pressure to dryness. The residue was purified by prep-HPLC to give Compound 367 (4.0 mg, yield 10.3%) as a white solid. ¹H NMR (400 MHz, CD₃OD) δ 8.50 (s, 1H), 8.22-8.18 (m, 2H), 7.88 (dd, J=7.8, 1.5 Hz, 1H), 7.74 (dd, J=12.3, 7.7 Hz, 2H), 7.66 (d, J=7.3 Hz, 1H), 7.59 (t, J=7.6 Hz, 1H), 7.51 (d, J=1.3 Hz, 1H), 7.47 (d, J=7.8 Hz, 1H), 7.36 (dd, J=7.6, 5.9 Hz, 1H), 5.24 (q, J=7.3 Hz, 1H), 4.89 (d, J=3.7 Hz, 1H), 4.51 (d, J=16.5 Hz, 1H), 4.29 (d, J=16.5 Hz, 1H), 3.77-3.74 (m, 2H), 3.65 (dd, J=9.6, 5.3 Hz, 2H), 3.57 (d, J=10.5 Hz, 1H), 3.16 (s, 2H), 2.76 (s, 6H), 2.70 (d, J=12.9 Hz, 1H), 2.15 (dd, J=13.4, 3.6 Hz, 1H), 1.58 (d, J=7.0 Hz, 3H), 1.41 (dd, J=5.7, 2.6 Hz, 1H), 1.05 (t, J=5.8 Hz, 1H). LCMS (ESI) (m/z): 665 (M+H)⁺.

### Scheme 347: Synthesis of (2S,4R)—N—((R)-1-(4-carbamimidoylthiophen-2-yl)ethyl)-1-((9,9-dimethyl-9H-fluorene-2-carbonyl)glycyl)-4-fluoro-4-(fluoromethyl)pyrrolidine-2-carboxamide (Compound 368)

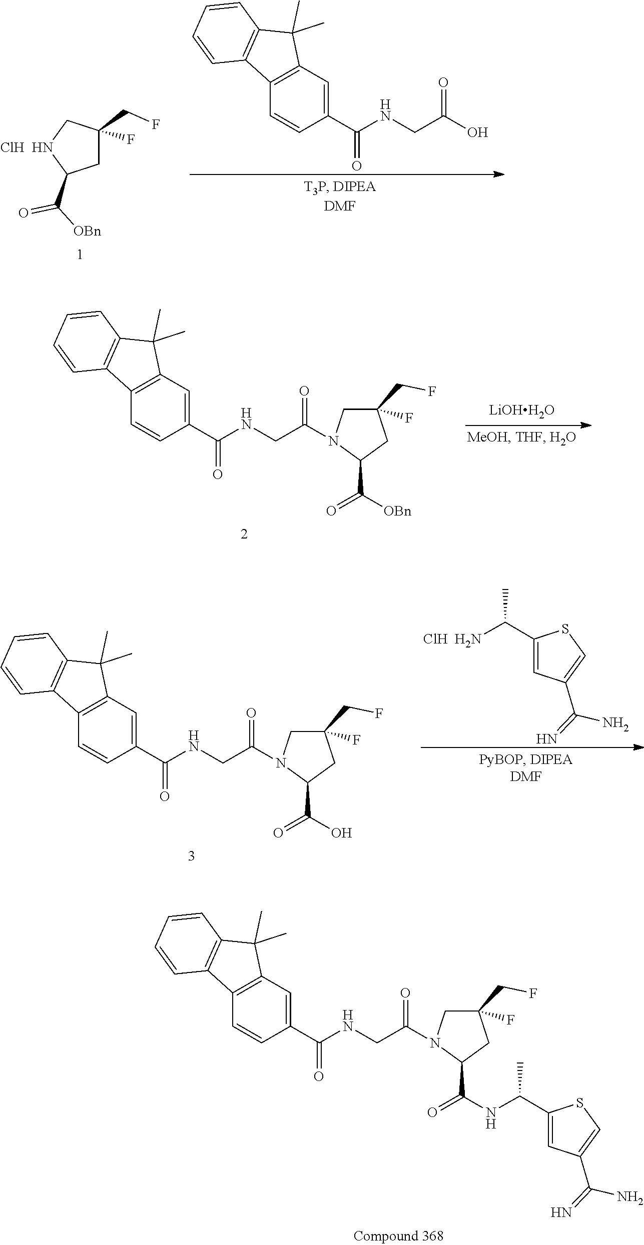

Compound 368 was prepared from benzyl (2S,4R)-4-fluoro-4-(fluoromethyl)pyrrolidine-2-carboxylate hydrochloride, (9,9-dimethyl-9H-fluorene-2-carbonyl)glycine, and (R)-5-(1-aminoethyl)thiophene-3-carboximidamide hydrochloride based on the procedures set forth in Scheme 3. ¹H NMR (400 MHz, CD₃OD) δ 8.52 (s, 1H), 8.23 (t, J=9.5 Hz, 1H), 8.00 (d, J=10.0 Hz, 1H), 7.88-7.82 (m, 3H), 7.58-7.51 (m, 2H), 7.39-7.35 (m, 2H), 5.28 (q, J=7.0 Hz, 1H), 4.76 (s, 1H), 4.66 (dd, J=13.7, 5.2 Hz, 2H), 4.35 (d, J=16.6 Hz, 1H), 4.17 (dd, J=14.3, 7.1 Hz, 2H), 3.97 (dd, J=32.0, 12.4 Hz, 1H), 2.78-2.39 (m, 1H), 2.22 (ddd, J=35.1, 14.2, 9.4 Hz, 1H), 1.61 (t, J=16.3 Hz, 3H), 1.51 (d, J=3.2 Hz, 6H). LCMS (ESI) (m/z): 594 (M+H)⁺.

### Scheme 348: Synthesis of (2S,4R)—N—((R)-1-(4-carbamimidoylthiophen-2-yl)ethyl)-1-((9,9-dimethyl-9H-fluorene-2-carbonyl)glycyl)-4-(methylsulfonyl)pyrrolidine-2-carboxamide (Compound 369)

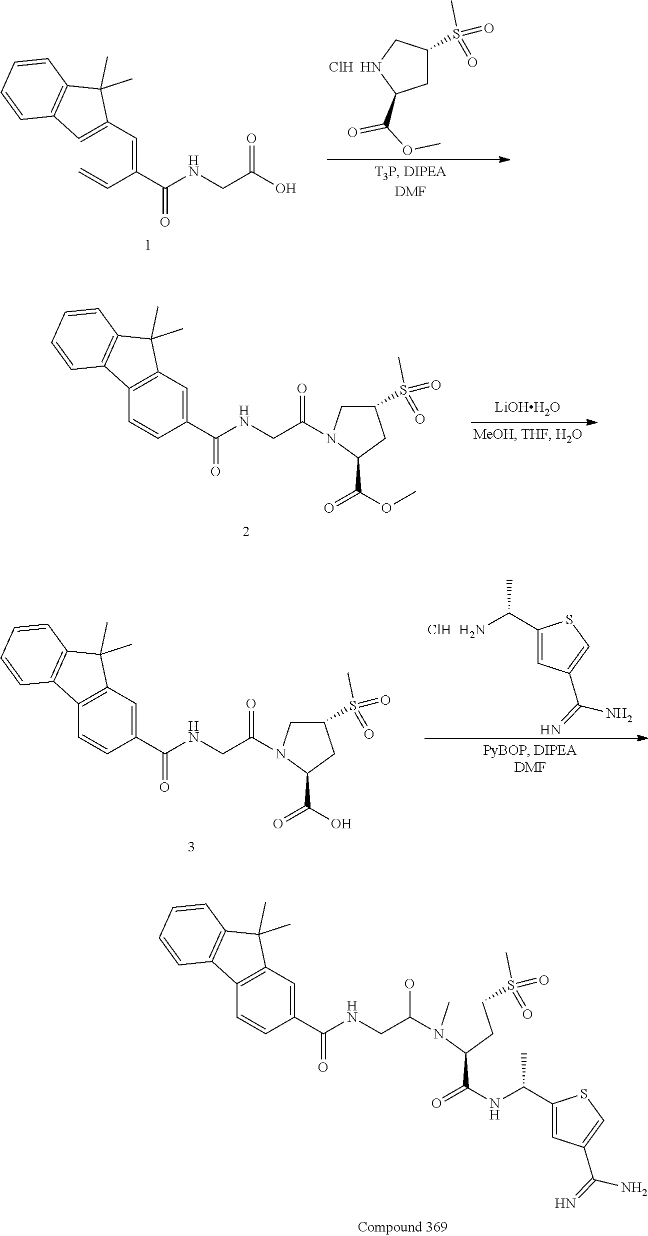

Compound 369 was prepared from (9,9-dimethyl-9H-fluorene-2-carbonyl)glycine, methyl (2S,4R)-4-(methylsulfonyl)pyrrolidine-2-carboxylate hydrochloride, and (R)-5-(1-aminoethyl)thiophene-3-carboximidamide hydrochloride based on the procedures set forth in Scheme 66. ¹H NMR (400 MHz, CD₃OD) δ 8.50 (s, 1H), 8.24 (dd, J=15.8, 1.6 Hz, 1H), 8.00 (d, J=8.8 Hz, 1H), 7.88-7.81 (m, 3H), 7.57-7.50 (m, 2H), 7.39-7.35 (m, 2H), 5.30 (dt, J=13.4, 6.5 Hz, 1H), 4.81-4.71 (m, 1H), 4.30 (s, 1H), 4.26-3.93 (m, 4H), 3.07 (d, J=18.1 Hz, 3H), 2.94-2.73 (m, 1H), 2.60-2.40 (m, 1H), 1.64 (dd, J=31.2, 7.0 Hz, 3H), 1.51 (d, J=3.5 Hz, 6H). LCMS (ESI) (m/z): 622 (M+H)⁺.

### Scheme 349: Synthesis of (2S,4R)—N—((R)-1-(4-carbamimidoylthiophen-2-yl)ethyl)-1-((9,9-difluoro-9H-fluorene-3-carbonyl)glycyl)-4-(pyridin-4-yl)pyrrolidine-2-carboxamide (Compound 370)

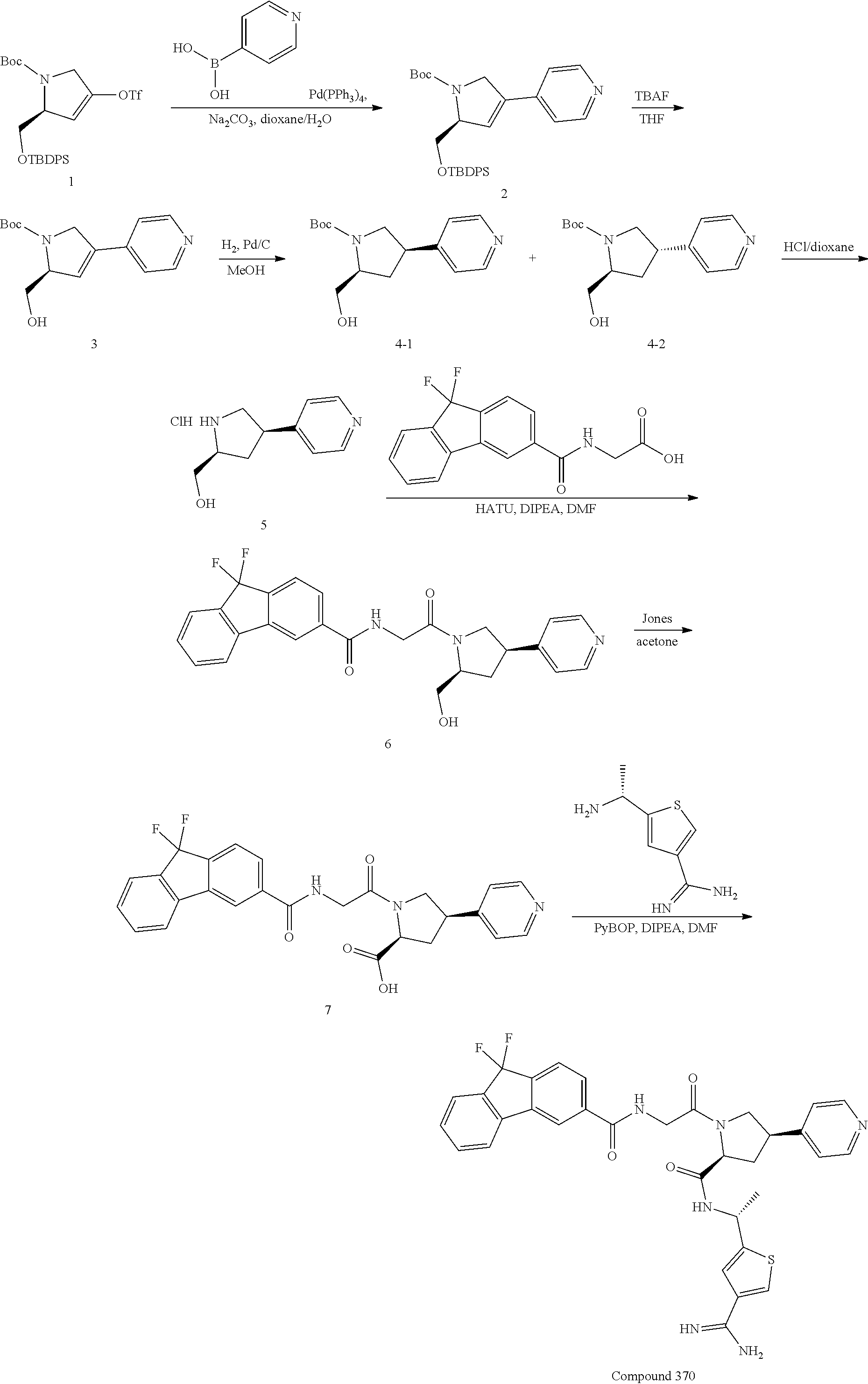

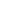

Compound 370 was prepared from tert-butyl (S)-2-(((tert-butyldiphenylsilyl)oxy)methyl)-4-(((trifluoromethyl)sulfonyl)oxy)-2,5-dihydro-1H-pyrrole-1-carboxylate, pyridin-4-ylboronic acid, (9,9-difluoro-9H-fluorene-3-carbonyl)glycine, and (R)-5-(1-aminoethyl)thiophene-3-carboximidamide based on the procedures set forth in Scheme 337. ¹H NMR (400 MHz, CD₃OD) δ 8.52 (s, 1H), 8.50 (s, 1H), 8.26-8.17 (m, 3H), 7.90 (d, J=9.1 Hz, 1H), 7.78-7.72 (m, 2H), 7.65 (s, 1H), 7.57 (d, J=15.7 Hz, 2H), 7.46 (t, J=7.2 Hz, 3H), 5.30 (d, J=7.6 Hz, 1H), 4.59-4.58 (m, 1H), 4.44 (d, J=16.7 Hz, 1H), 4.35 (s, 1H), 4.19 (d, J=16.8 Hz, 1H), 3.77-3.66 (m, 2H), 2.80 (d, J=6.3 Hz, 1H), 2.19-2.07 (m, 1H), 1.60 (d, J=7.0 Hz, 3H). LCMS (ESI) m/z=629 (M+H)⁺.

### Scheme 350: Synthesis of (2S,4R)—N—((R)-1-(4-carbamimidoylthiophen-2-yl)ethyl)-4-fluoro-4-(fluoromethyl)-1-((9-methyl-9H-fluorene-2-carbonyl)glycyl)pyrrolidine-2-carboxamide (Compound 371)

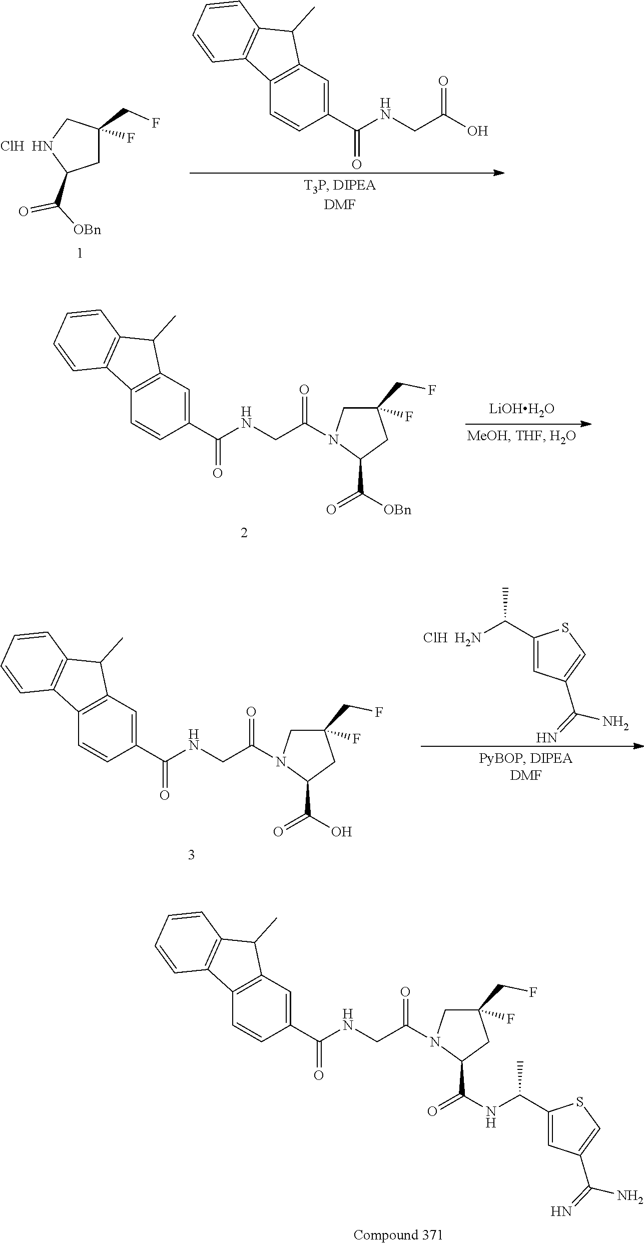

Compound 371 was prepared from benzyl (2S,4R)-4-fluoro-4-(fluoromethyl)pyrrolidine-2-carboxylate hydrochloride, (9-methyl-9H-fluorene-2-carbonyl)glycine, and (R)-5-(1-aminoethyl)thiophene-3-carboximidamide hydrochloride based on the procedures set forth in Scheme 3. ¹H NMR (400 MHz, CD₃OD) δ 8.51 (s, 1H), 8.22 (dd, J=9.8, 8.5 Hz, 1H), 8.04 (d, J=9.2 Hz, 1H), 7.90-7.84 (m, 3H), 7.60-7.54 (m, 2H), 7.41-7.36 (m, 2H), 5.31 (dq, J=13.9, 7.1 Hz, 1H), 4.81-4.73 (m, 1H), 4.71-4.61 (m, 2H), 4.34 (d, J=16.7 Hz, 1H), 4.15 (dd, J=17.7, 10.0 Hz, 2H), 4.03-3.90 (m, 2H), 2.74-2.46 (m, 1H), 2.23 (ddd, J=34.7, 14.2, 9.1 Hz, 1H), 1.61 (t, J=16.0 Hz, 3H), 1.54 (dd, J=7.4, 4.0 Hz, 3H). LCMS (ESI) (m/z): 580 (M+H)⁺.

### Scheme 351: Synthesis of (2S,4R)—N—((R)-1-(4-carbamimidoylthiophen-2-yl)ethyl)-1-((dibenzo[b,d]furan-3-carbonyl)glycyl)-4-fluoro-4-(fluoromethyl)pyrrolidine-2-carboxamide (Compound 372)

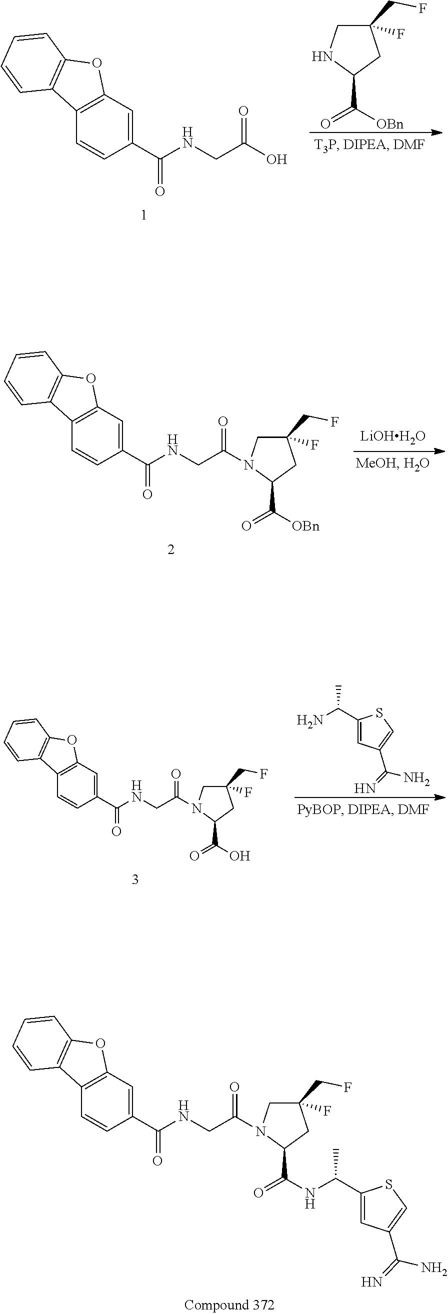

Compound 372 was prepared from dibenzo[b,d]furan-3-carbonyl)glycine, benzyl (2S,4R)-4-fluoro-4-(fluoromethyl)pyrrolidine-2-carboxylate, and (R)-5-(1-aminoethyl)thiophene-3-carboximidamide based on the procedures provided in Scheme 3. ¹H NMR (400 MHz, CD₃OD) δ 8.23 (dd, J=23.3, 1.6 Hz, 1H), 8.10 (d, J=10.6 Hz, 3H), 7.91-7.86 (m, 1H), 7.62 (s, 1H), 7.59-7.54 (m, 2H), 7.42 (t, J=7.5 Hz, 1H), 5.28 (d, J=6.6 Hz, 1H), 4.81-4.76 (m, 1H), 4.71-4.61 (m, 2H), 4.36 (d, J=16.7 Hz, 1H), 4.21-4.11 (m, 2H), 3.97 (dd, J=32.1, 12.2 Hz, 1H), 2.79-2.55 (m, 1H), 2.23 (ddd, J=34.8, 14.2, 9.2 Hz, 1H), 1.59 (d, J=6.9 Hz, 3H). LCMS (ESI) (m/z): 568 (M+H)⁺.

### Scheme 352: Synthesis of (2S,4R)—N—((R)-1-(4-carbamimidoylthiophen-2-yl)ethyl)-4-fluoro-4-(methoxymethyl)-1-((phenoxathiine-3-carbonyl)glycyl)pyrrolidine-2-carboxamide (Compound 373)

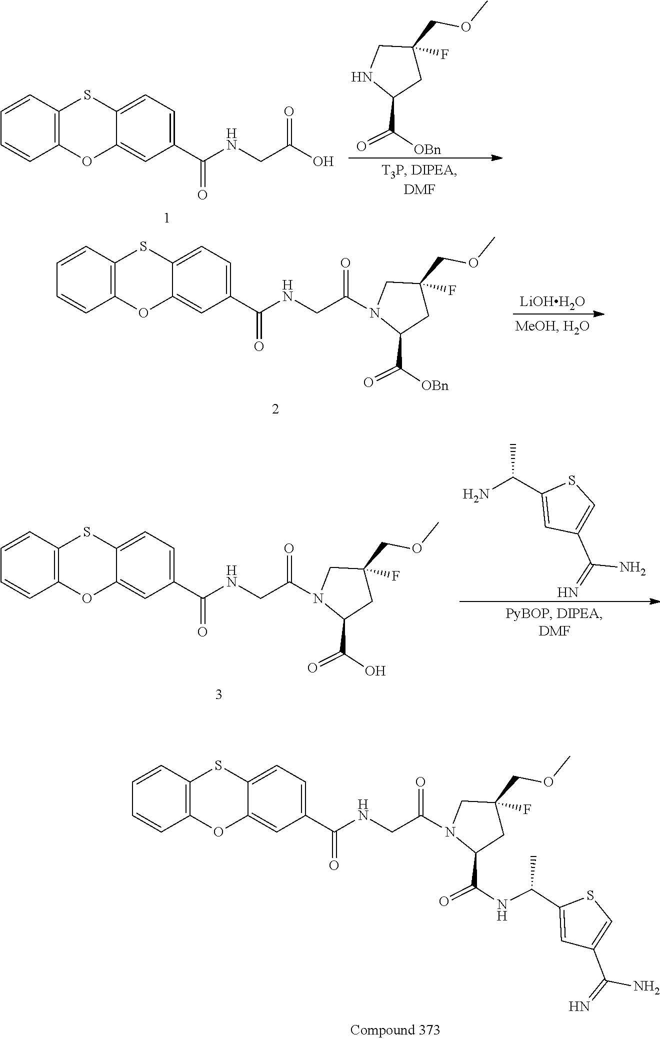

Compound 373 was prepared from (phenoxathiine-3-carbonyl)glycine, benzyl (2S,4R)-4-fluoro-4-(methoxymethyl)pyrrolidine-2-carboxylate, and (R)-5-(1-aminoethyl)thiophene-3-carboximidamide based on the procedures set forth in Scheme 3. ¹H NMR (400 MHz, CD₃OD) δ 8.48 (s, 1H), 8.21 (d, J=1.6 Hz, 1H), 7.54 (dd, J=5.4, 3.9 Hz, 2H), 7.49 (d, J=1.8 Hz, 1H), 7.23 (d, J=8.0 Hz, 1H), 7.21-7.14 (m, 2H), 7.10-7.02 (m, 2H), 5.27 (d, J=7.1 Hz, 1H), 4.64-4.58 (m, 1H), 4.28 (d, J=16.7 Hz, 1H), 4.10 (d, J=16.7 Hz, 2H), 3.89 (dd, J=33.6, 12.1 Hz, 1H), 3.70 (dd, J=17.6, 5.6 Hz, 2H), 3.43 (d, J=5.4 Hz, 3H), 2.60-2.45 (m, 1H), 2.19 (ddd, J=36.8, 14.1, 9.6 Hz, 1H), 1.58 (d, J=7.0 Hz, 3H). LC/MS (ESI) m/z: 612 (M+H)⁺.

### Scheme 353: Synthesis of (S)-7-((9H-carbazole-2-carbonyl)glycyl)-N—((R)-1-(4-carbamimidoylthiophen-2-yl)ethyl)-1,4-dioxa-7-azaspiro[4.4]nonane-8-carboxamide (Compound 374)

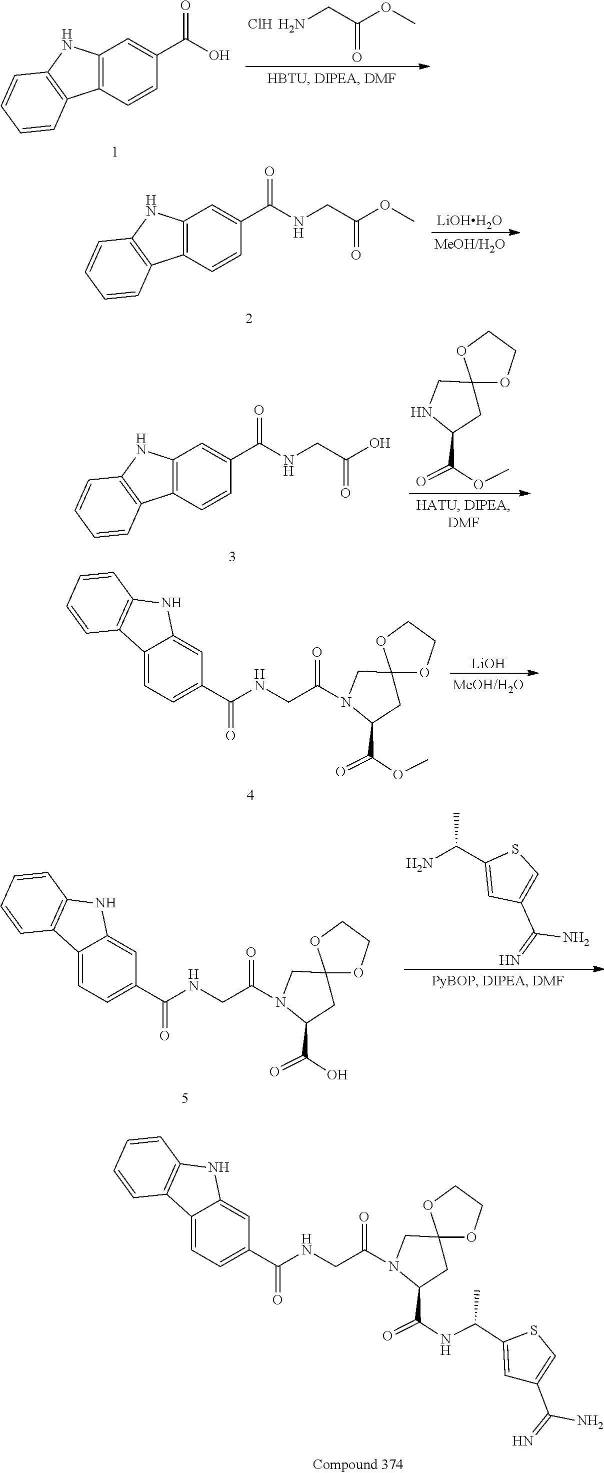

Compound 374 was prepared from 9H-carbazole-2-carboxylic acid, methyl glycinate hydrochloride, methyl (S)-1,4-dioxa-7-azaspiro[4.4]nonane-8-carboxylate, and (R)-5-(1-aminoethyl)thiophene-3-carboximidamide based on the procedures set forth in Scheme 4. ¹H NMR (400 MHz, CD₃OD) δ 8.53 (s, 1H), 8.23 (dd, J=14.2, 1.6 Hz, 1H), 8.12 (t, J=8.1 Hz, 2H), 7.99 (t, J=3.5 Hz, 1H), 7.66 (dd, J=8.2, 1.5 Hz, 1H), 7.54-7.41 (m, 3H), 7.21 (dd, J=10.8, 4.0 Hz, 1H), 5.28 (q, J=7.1 Hz, 1H), 4.57 (d, J=5.4 Hz, 1H), 4.21 (d, J=2.9 Hz, 1H), 4.04-3.96 (m, 4H), 3.85-3.77 (m, 2H), 2.43 (dt, J=18.3, 9.2 Hz, 1H), 2.21 (dd, J=13.2, 6.6 Hz, 1H), 1.58 (d, J=7.0 Hz, 3H). LCMS (ESI) m/z=575 (M+H)⁺.

### Scheme 354: Synthesis of (2S,4R)—N—((R)-1-(4-carbamimidoylthiophen-2-yl)ethyl)-1-((5,5-dioxidodibenzo[b,d]thiophene-3-carbonyl)glycyl)-4-fluoro-4-(fluoromethyl)pyrrolidine-2-carboxamide (Compound 375)

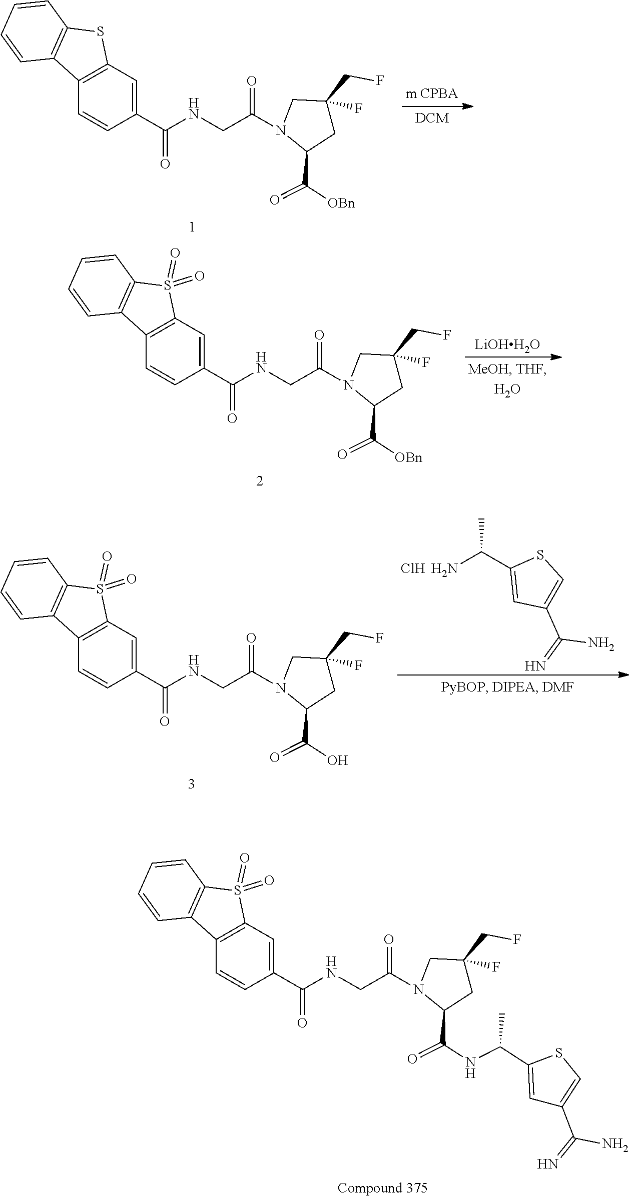

Compound 375 was prepared from benzyl (2S,4R)-1-((dibenzo[b,d]thiophene-3-carbonyl)glycyl)-4-fluoro-4-(fluoromethyl)pyrrolidine-2-carboxylate and (R)-5-(1-aminoethyl)thiophene-3-carboximidamide hydrochloride based on the procedures set forth in Scheme 251. ¹H NMR (400 MHz, CD₃OD) δ 8.48 (s, 1H), 8.31 (dd, J=13.0, 1.3 Hz, 1H), 8.26-8.20 (m, 2H), 8.13 (dd, J=13.2, 7.9 Hz, 2H), 7.89 (d, J=7.6 Hz, 1H), 7.80 (td, J=7.6, 1.0 Hz, 1H), 7.69 (td, J=7.6, 0.7 Hz, 1H), 7.57-7.53 (m, 1H), 5.39-5.25 (m, 1H), 4.81-4.76 (m, 1H), 4.65 (dd, J=20.9, 12.7 Hz, 2H), 4.36 (d, J=16.8 Hz, 1H), 4.19-4.08 (m, 2H), 4.03-3.90 (m, 1H), 2.82-2.55 (m, 1H), 2.23 (ddd, J=35.2, 14.3, 9.3 Hz, 1H), 1.63 (dd, J=31.9, 7.0 Hz, 3H). LCMS (ESI) (m/z): 616 (M+H)⁺.

### Scheme 355: Synthesis of (S)—N—((R)-1-(4-carbamimidoylthiophen-2-yl)-2-hydroxyethyl)-7-((9,9-difluoro-9H-fluorene-3-carbonyl)glycyl)-1,4-dioxa-7-azaspiro [4.4]nonane-8-carboxamide (Compound 376)

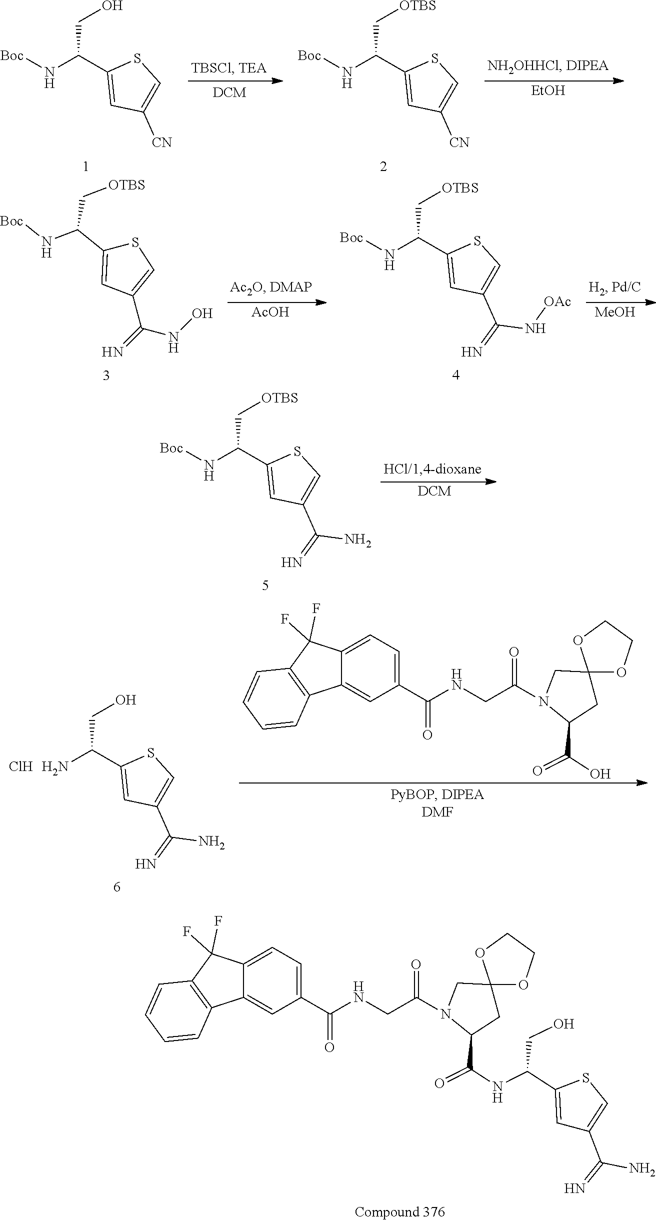

### Step 1: tert-butyl (R)-(2-((tert-butyldimethylsilyl)oxy)-1-(4-cyanothiophen-2-yl)ethyl)carbamate

To a solution of tert-butyl (R)-(1-(4-cyanothiophen-2-yl)-2-hydroxyethyl)carbamate (3.0 g, 11.15 mmol) in DCM (30 mL) was added TBSCl (2.53 g, 16.73 mmol), DMAP (272 mg, 2.23 mmol) and TEA (6.20 mL, 44.60 mmol) at 0° C., and the mixture was stirred at 28° C. for 16 hours. The mixture was diluted with water and extracted with DCM twice. The combined organic layers were washed with brine, dried with anhydrous Na₂SO₄, filtered, and concentrated to dryness. The residue was purified by flash chromatography on silica gel (0-50% EtOAc in PE) to give tert-butyl (R)-(2-((tert-butyldimethylsilyl)oxy)-1-(4-cyanothiophen-2-yl)ethyl)carbamate (3.9 g, yield 91.2%) as a white solid. LC/MS (ESI) m/z: 383(M+H)⁺.

### Step 2: tert-butyl (R)-(2-((tert-butyldimethylsilyl)oxy)-1-(4-(N-hydroxycarbamimidoyl)thiophen-2-yl)ethyl)carbamate

To a solution of tert-butyl (R)-(2-((tert-butyldimethylsilyl)oxy)-1-(4-cyanothiophen-2-yl)ethyl)carbamate (3.0 g, 7.85 mmol) in EtOH (30 mL) was added NH₂OH·HCl (1.36 g, 19.63 mmol) and DIPEA (3.9 mL, 23.56 mmol) and the reaction mixture was stirred at room temperature for 16 hours. The mixture was diluted with EtOAc, washed with water and brine, dried over anhydrous Na₂SO₄, filtered, and concentrated under reduced pressure to dryness. The residue was purified by flash chromatography on silica gel (0-7% MeOH in DCM) to give tert-butyl (R)-(2-((tert-butyldimethylsilyl)oxy)-1-(4-(N-hydroxycarbamimidoyl)thiophen-2-yl)ethyl)carbamate (2.76 g, yield 84.7%) as a light-yellow oil. LC/MS (ESI) m/z: 416 (M+H)⁺.

### Step 3: tert-butyl (R)-(1-(4-(N-acetoxycarbamimidoyl)thiophen-2-yl)-2-((tert-butyldimethylsilyl)oxy)ethyl)carbamate

To a solution of tert-butyl (R)-(2-((tert-butyldimethylsilyl)oxy)-1-(4-(N-hydroxycarbamimidoyl)thiophen-2-yl)ethyl)carbamate (2.76 g, 6.65 mmol) in AcOH (20 mL) was added Ac₂O (20 mL) and DMAP (243 mg, 2.00 mmol) and the reaction mixture was stirred at room temperature for 16 hours. The mixture was diluted with EtOAc, washed with water and brine, dried over anhydrous Na₂SO₄, filtered, and concentrated under reduced pressure to dryness. The residue was purified by flash chromatography on silica gel (0-4% MeOH in DCM) to give tert-butyl (R)-(1-(4-(N-acetoxycarbamimidoyl)thiophen-2-yl)-2-((tert-butyldimethylsilyl)oxy)ethyl)carbamate (2.69 g, yield 88.8%) as a yellow oil. LC/MS (ESI) m/z: 458 (M+H)⁺.

### Step 4: tert-butyl (R)-(2-((tert-butyldimethylsilyl)oxy)-1-(4-carbamimidoylthiophen-2-yl)ethyl)carbamate

To a solution of tert-butyl (R)-(1-(4-(N-acetoxycarbamimidoyl)thiophen-2-yl)-2-((tert-butyldimethylsilyl)oxy)ethyl)carbamate (2.69 g, 5.89 mmol) in MeOH (40 mL) was added Pd/C (300 mg, 10% wt.) under N₂ atmosphere and the reaction mixture was stirred under a H₂ balloon at room temperature for 16 hours. The mixture was filtered, and the filtrate was concentrated under reduced pressure to dryness to give tert-butyl (R)-(2-((tert-butyldimethylsilyl)oxy)-1-(4-carbamimidoylthiophen-2-yl)ethyl)carbamate (2.29 g, yield 97.5%) as a white solid. LC/MS (ESI) m/z: 400 (M+H)⁺.

### Step 5: (R)-5-(1-amino-2-hydroxyethyl)thiophene-3-carboximidamide hydrochloride

To a solution of tert-butyl (R)-(2-((tert-butyldimethylsilyl)oxy)-1-(4-carbamimidoylthiophen-2-yl)ethyl)carbamate (2.29 g, 5.74 mmol) in EtOAc (20 mL) was added HCl/1,4-dioxane (20 mL, 4M) under N₂ atmosphere and the reaction mixture was stirred at room temperature for 2 hours. The mixture was concentrated under reduced pressure to dryness to give (R)-5-(1-amino-2-hydroxyethyl)thiophene-3-carboximidamide hydrochloride (1.14 g, yield 89.9%) as a white solid. LC/MS (ESI) m/z: 186 (M+H)⁺.

### Step 6: (S)—N—((R)-1-(4-carbamimidoylthiophen-2-yl)-2-hydroxyethyl)-7-((9,9-difluoro-9H-fluorene-3-carbonyl)glycyl)-1,4-dioxa-7-azaspiro[4.4]nonane-8-carboxamide (Compound 376)

To a mixture of (S)-7-((9,9-difluoro-9H-fluorene-3-carbonyl)glycyl)-1,4-dioxa-7-azaspiro[4.4]nonane-8-carboxylic acid (40 mg, 0.087 mmol) and (R)-5-(1-amino-2-hydroxyethyl)thiophene-3-carboximidamide hydrochloride (24 mg, 0.11 mmol) in DMF (3 mL) was added DIPEA (56 mg, 0.43 mmol) and PyBOP (68 mg, 0.13 mmol) under N₂ atmosphere and the reaction mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 376 (5.1 mg, yield 9.3%) as a white solid. ¹H NMR (400 MHz, CD₃OD) δ 8.22 (d, J=21.6 Hz, 2H), 7.89 (d, J=7.7 Hz, 1H), 7.75 (dd, J=16.4, 7.7 Hz, 2H), 7.67 (d, J=8.2 Hz, 1H), 7.62-7.56 (m, 2H), 7.46 (t, J=7.5 Hz, 1H), 5.39-5.18 (m, 1H), 4.66-4.60 (m, 1H), 4.28-4.13 (m, 2H), 4.09-3.93 (m, 5H), 3.86-3.81 (m, 3H), 2.53-2.39 (m, 1H), 2.24 (dd, J=13.5, 6.4 Hz, 1H). LC/MS (ESI) m/z: 626 (M+H)⁺.

### Scheme 356: Synthesis of (2S,4S)—N—((R)-1-(4-carbamimidoylthiophen-2-yl)ethyl)-1-((9,9-difluoro-9H-fluorene-3-carbonyl)glycyl)-4-(pyridin-4-yl)pyrrolidine-2-carboxamide (Compound 377)

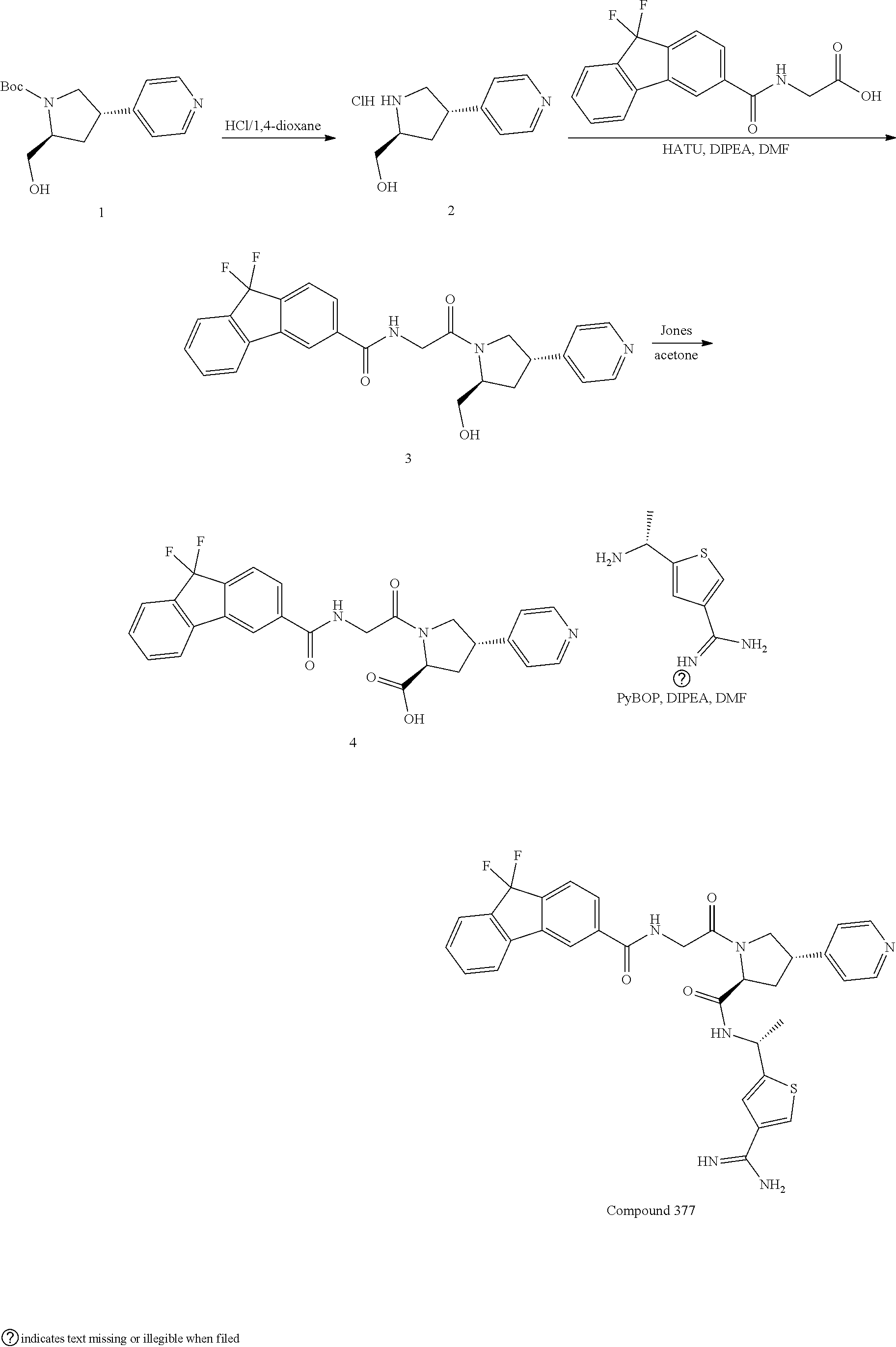

Compound 377 was prepared from tert-butyl (2S,4S)-2-(hydroxymethyl)-4-(pyridin-4-yl)pyrrolidine-1-carboxylate, (9,9-difluoro-9H-fluorene-3-carbonyl)glycine, and (R)-5-(1-aminoethyl)thiophene-3-carboximidamide based on the procedures set forth in Scheme 307. ¹H NMR (400 MHz, CD₃OD) δ 8.53 (s, 2H), 8.27-8.18 (m, 2H), 7.89 (dt, J=9.3, 4.6 Hz, 1H), 7.75 (dd, J=17.2, 7.7 Hz, 2H), 7.66 (d, J=7.3 Hz, 1H), 7.59 (t, J=7.5 Hz, 1H), 7.47 (dd, J=17.6, 10.9 Hz, 4H), 5.35-5.30 (m, 1H), 4.71 (t, J=5.5 Hz, 1H), 4.37-4.16 (m, 3H), 3.80 (d, J=3.2 Hz, 2H), 2.47 (s, 1H), 2.04 (s, 1H), 1.63 (t, J=16.7 Hz, 3H). LCMS (ESI) m/z: 629 (M+H)⁺.

Scheme 357: Synthesis of (2S,4R)—N—((R)-1-(4-carbamimidoylthiophen-2-yl)ethyl)-1-((dibenzo[b,d]thiophene-3-carbonyl)glycyl)-4-(methylsulfonyl)pyrrolidine-2-carboxamide (Compound 378)

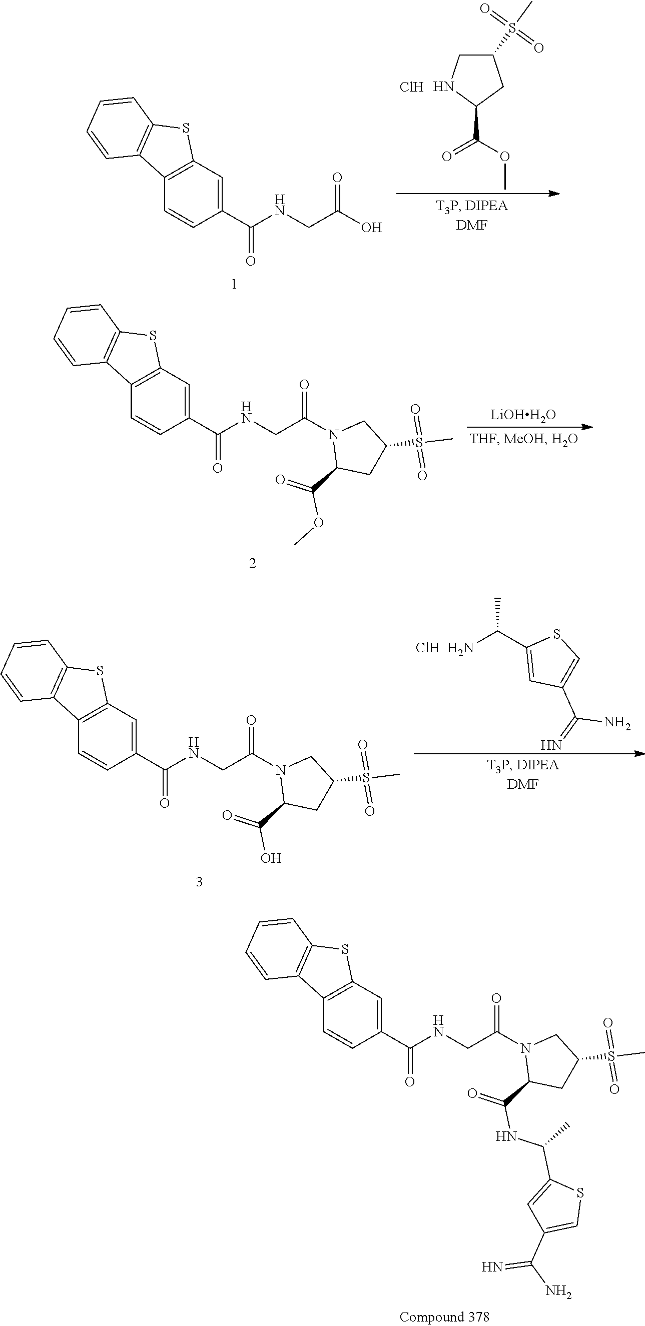

Compound 378 was prepared from (dibenzo[b,d]thiophene-3-carbonyl)glycine, methyl (2S,4R)-4-(methylsulfonyl)pyrrolidine-2-carboxylate hydrochloride, and (R)-5-(1-aminoethyl)thiophene-3-carboximidamide hydrochloride based on the procedures set forth in Scheme 66. ¹H NMR (400 MHz, CD₃OD) δ 8.51 (s, 1H), 8.40 (dd, J=5.1, 1.3 Hz, 1H), 8.33 (dt, J=6.4, 1.9 Hz, 2H), 8.23 (dd, J=15.6, 1.6 Hz, 1H), 7.95 (ddd, J=8.5, 5.7, 1.3 Hz, 2H), 7.58-7.50 (m, 3H), 5.40-5.22 (m, 1H), 4.73 (dd, J=8.7, 4.9 Hz, 1H), 4.32 (s, 2H), 4.18 (dt, J=22.5, 12.8 Hz, 3H), 3.07 (d, J=18.9 Hz, 3H), 2.89-2.75 (m, 1H), 2.56-2.41 (m, 1H), 1.62 (t, J=15.9 Hz, 3H). LC/MS (ESI) m/z: 612 (M+H)⁺.

### Scheme 358: Synthesis of (2S,4R)—N—((R)-1-(4-carbamimidoylthiophen-2-yl)ethyl)-1-((dibenzo[b,d]furan-3-carbonyl)glycyl)-4-(methylsulfonyl)pyrrolidine-2-carboxamide (Compound 379)

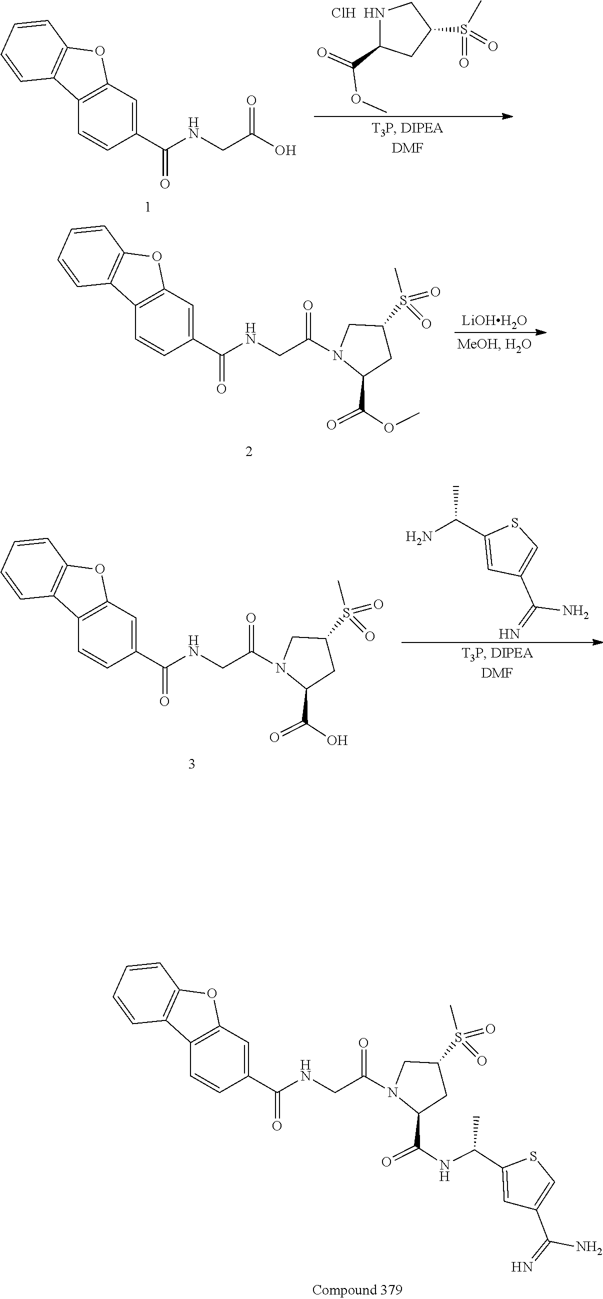

Compound 379 was prepared from (dibenzo[b,d]furan-3-carbonyl)glycine, methyl (2S,4R)-4-(methylsulfonyl)pyrrolidine-2-carboxylate hydrochloride, and (R)-5-(1-aminoethyl)thiophene-3-carboximidamide based on the procedures set forth in Scheme 66. ¹H NMR (400 MHz, CD₃OD) δ 8.52 (d, J=2.8 Hz, 1H), 8.23 (d, J=14.1 Hz, 1H), 8.11 (d, J=7.0 Hz, 3H), 7.92-7.88 (m, 1H), 7.64 (d, J=8.2 Hz, 1H), 7.58 (d, J=7.3 Hz, 1H), 7.51 (s, 1H), 7.42 (t, J=7.5 Hz, 1H), 5.27 (d, J=6.6 Hz, 1H), 4.73 (dd, J=8.8, 4.8 Hz, 1H), 4.32 (s, 2H), 4.19 (dd, J=19.2, 7.9 Hz, 3H), 3.07 (d, J=18.8 Hz, 3H), 2.81-2.73 (m, 1H), 2.50-2.38 (m, 1H), 1.64 (dd, J=31.7, 6.9 Hz, 3H). LCMS (ESI) (m/z): 596 (M+H)⁺.

### Scheme 359: Synthesis of (2S,4R)—N—((R)-1-(4-carbamimidoylthiophen-2-yl)-2-hydroxyethyl)-1-((9,9-dimethyl-9H-fluorene-3-carbonyl)glycyl)-4-(methylsulfonyl) pyrrolidine-2-carboxamide (Compound 380)

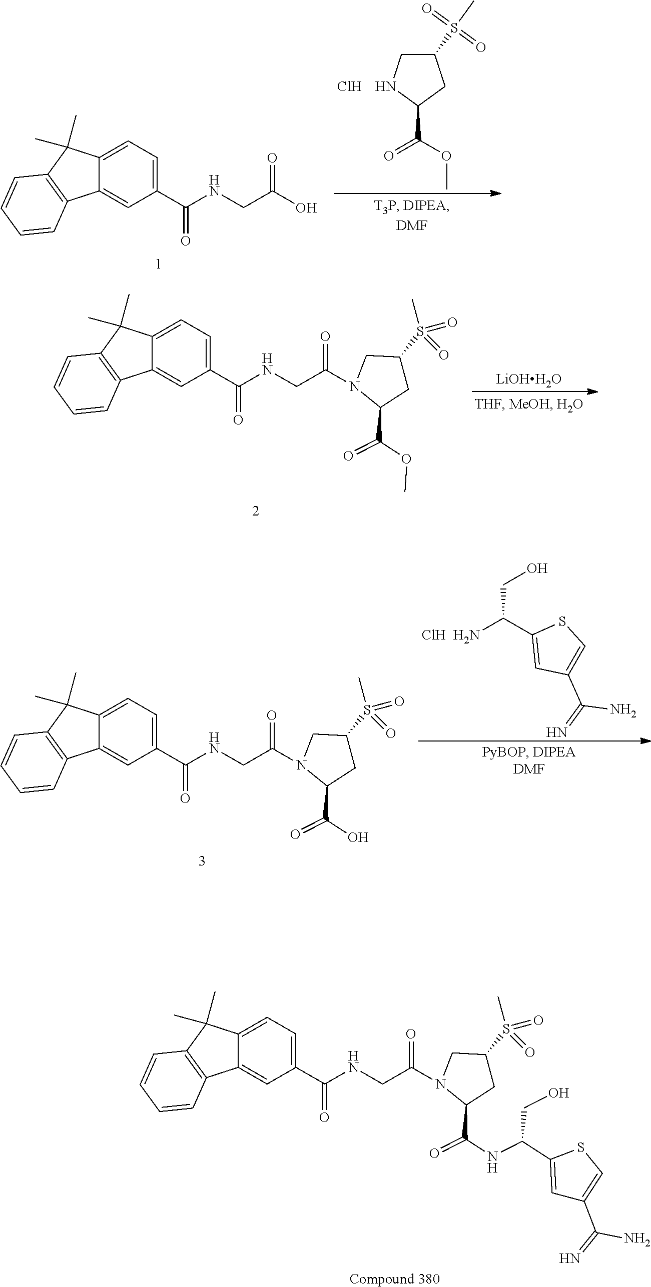

Compound 380 was prepared from (dibenzo[b,d]thiophene-3-carbonyl)glycine, methyl (2S,4R)-4-(methylsulfonyl)pyrrolidine-2-carboxylate hydrochloride, and (R)-5-(1-amino-2-hydroxyethyl)thiophene-3-carboximidamide hydrochloride based on the procedures set forth in Scheme 3. ¹H NMR (400 MHz, CD₃OD) δ 8.51 (s, 1H), 8.25 (ddd, J=6.2, 5.6, 1.5 Hz, 2H), 7.81 (ddd, J=11.3, 6.7, 2.1 Hz, 2H), 7.60-7.54 (m, 2H), 7.52-7.49 (m, 1H), 7.36 (dd, J=5.5, 3.2 Hz, 2H), 5.29 (dt, J=41.3, 5.8 Hz, 1H), 4.80 (dd, J=8.7, 4.7 Hz, 1H), 4.36-4.22 (m, 2H), 4.20-4.02 (m, 3H), 3.95-3.80 (m, 2H), 3.07 (d, J=18.6 Hz, 3H), 2.93-2.73 (m, 1H), 2.65-2.47 (m, 1H), 1.49 (s, 6H). LC/MS (ESI) m/z: 638 (M+H)⁺.

### Scheme 360: Synthesis of (2S,4R)—N—((R)-1-(4-carbamimidoylthiophen-2-yl)ethyl)-1-((5,5-dioxidodibenzo[b,d]thiophene-3-carbonyl)glycyl)-4-(methylsulfonyl) pyrrolidine-2-carboxamide (Compound 381)

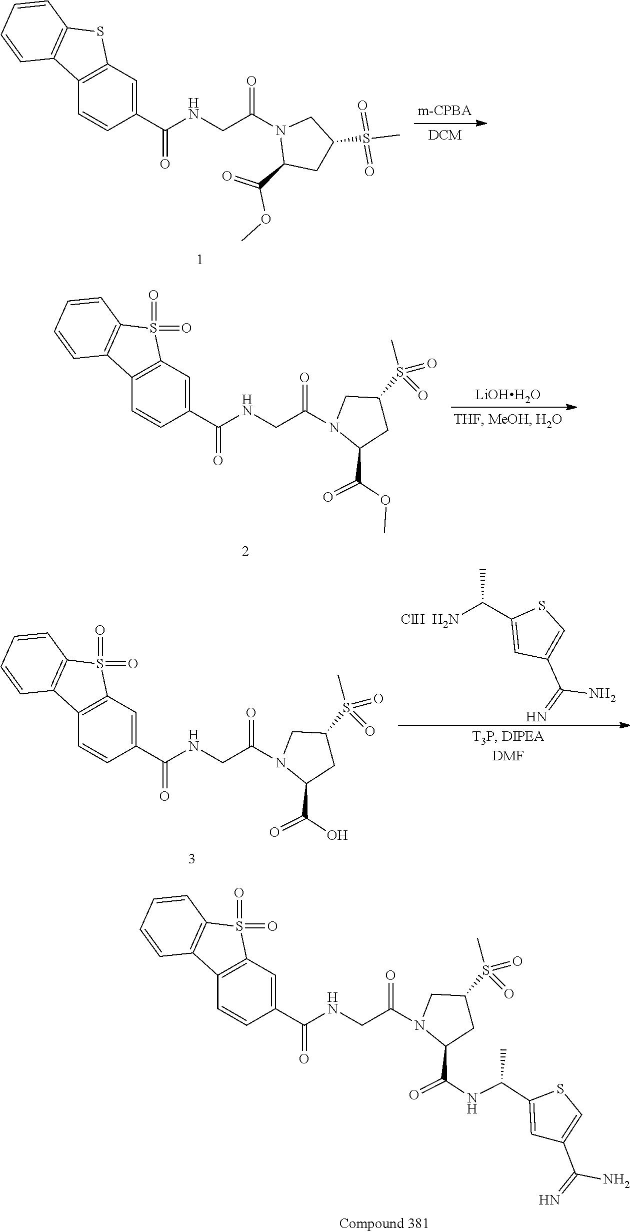

Compound 381 was prepared from methyl (2S,4R)-1-((dibenzo[b,d]thiophene-3-carbonyl)glycyl)-4-(methylsulfonyl)pyrrolidine-2-carboxylate and (R)-5-(1-aminoethyl)thiophene-3-carboximidamide hydrochloride based on the procedures set forth in Scheme 251. ¹H NMR (400 MHz, CD₃OD) δ 8.50 (s, 1H), 8.30 (dd, J=11.1, 1.2 Hz, 1H), 8.25-8.18 (m, 2H), 8.12 (dd, J=12.0, 7.9 Hz, 2H), 7.89 (d, J=7.6 Hz, 1H), 7.79 (td, J=7.6, 0.9 Hz, 1H), 7.71-7.66 (m, 1H), 7.52 (d, J=19.5 Hz, 1H), 5.31 (dq, J=13.5, 6.5 Hz, 1H), 4.79-4.70 (m, 1H), 4.32-3.98 (m, 5H), 3.07 (d, J=19.6 Hz, 3H), 2.92-2.73 (m, 1H), 2.59-2.40 (m, 1H), 1.63 (dd, J=32.1, 7.0 Hz, 3H). LC/MS (ESI) m/z: 644 (M+H)⁺.

### Scheme 361: Synthesis of (1S,3S,5R)-5-((3-aminopropoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 382)

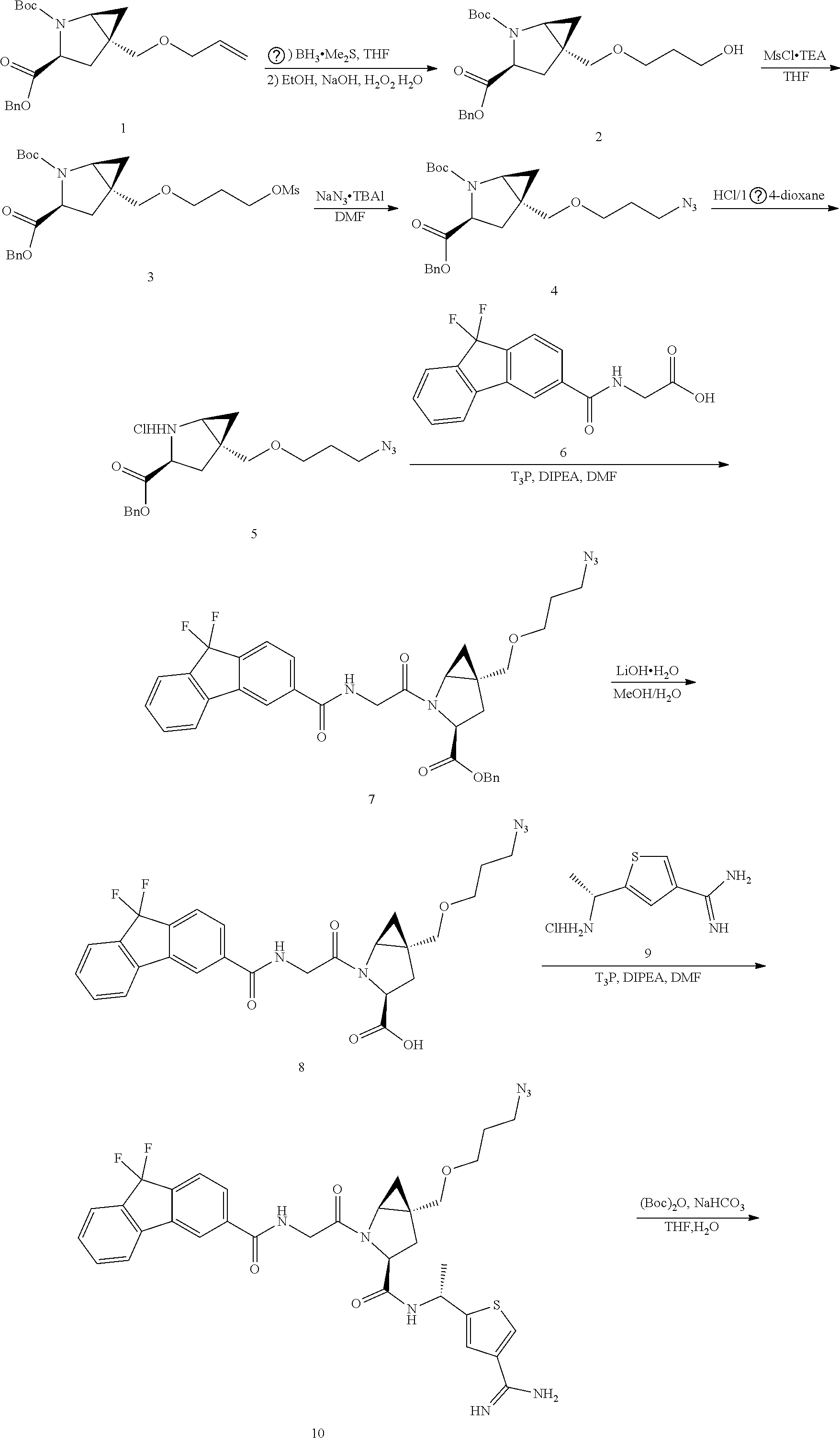

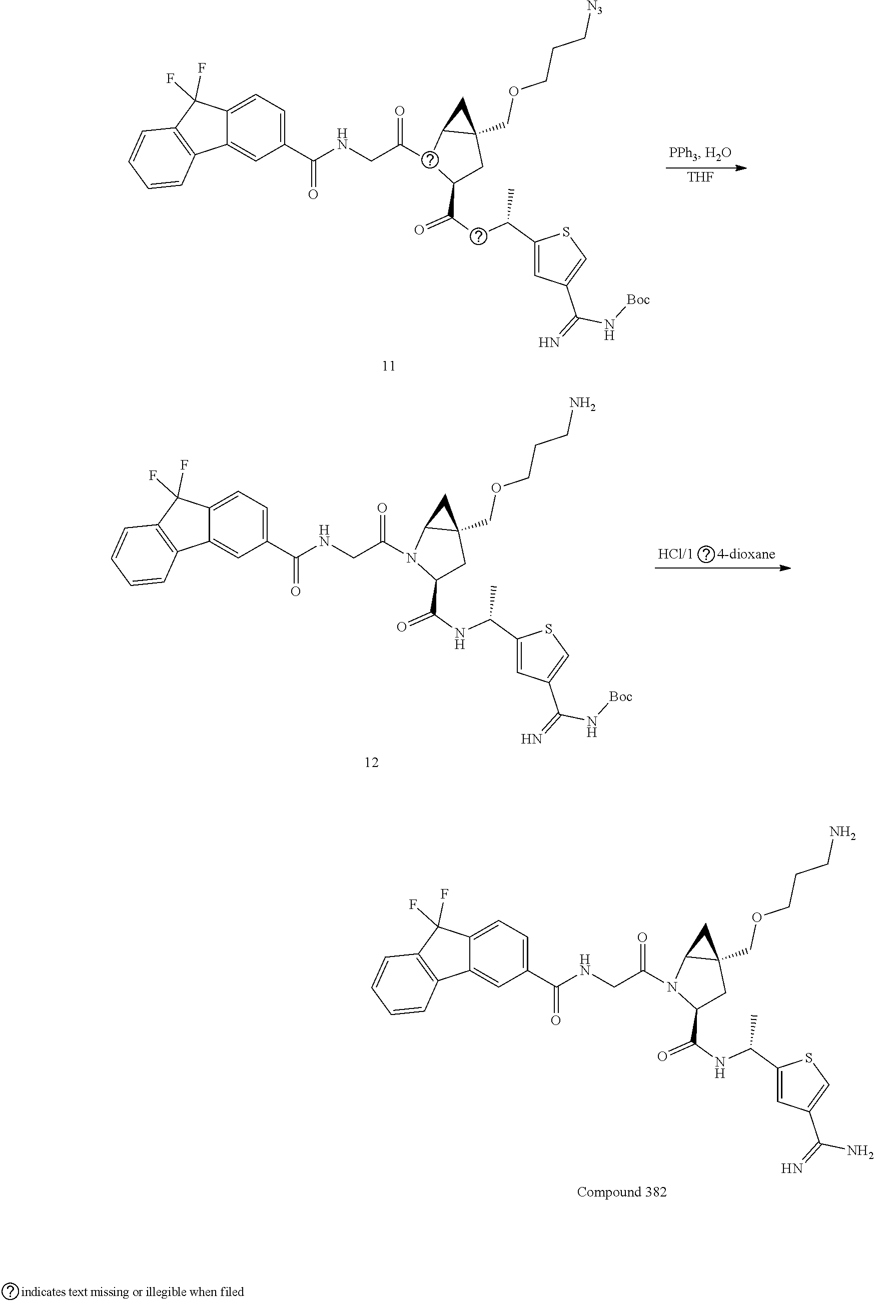 [SMILES: "THF" ; C[C@@H](CC(=O)[C@@H]1C[C@]2(COCCCN=[N+]=[N-])C[C@@H]2C1C(=O)CNC(=O)c1ccc2c(c1)-c1ccccc1C2(F)F)c1cc(C(=N)NC(=O)OC(C)(C)C)cs1 ; C[C@@H](NC(=O)[C@@H]1C[C@]2(COCCCN)C[C@@H]2N1C(=O)CNC(=O)c1ccc2c(c1)-c1ccccc1C2(F)F)c1cc(C(=N)NC(=O)OC(C)(C)C)cs1 ; C[C@@H](NC(=O)[C@@H]1C[C@]2(COCCCN)C[C@@H]2N1C(=O)CNC(=O)c1ccc2c(c1)-c1ccccc1C2(F)F)c1cc(C(=N)N)cs1]

### Step 1: 3-benzyl 2-(tert-butyl) (1S,3S,5R)-5-((3-hydroxypropoxy)methyl)-2-azabicycto[3.1.0]hexane-2,3-dicarboxylate

To a solution of 2-methyl-2-butene (146 mg, 2.08 mmol) in THF (2 mL) was added BH₃·Me₂S (1.1 mL, 1.1 mmol) drop-wisely at 0° C. and the reaction solution was stirred at 0° C. for 2 hours. The above mixture was added drop-wisely to a solution of 3-benzyl 2-(tert-butyl) (1 S,3S,5R)-5-((allyloxy)methyl)-2-azabicyclo[3.1.0]hexane-2,3-dicarboxylate (200 mg, 0.52 mmol) in THF (4 mL) at 0° C. and the resulting mixture was stirred at room temperature for 20 hours. To the above mixture, Ethanol (1.4 mL) was added at 0° C. followed by drop-wise addition of aq·NaOH solution (3M, 0.9 mL) and H₂O₂ (1.5 mL, 30% w.t.) successively at −10° C. The resulting mixture was stirred at room temperature for 3 hours. The layers were separated, and the aqueous layer was extracted with MTBE twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give 3-benzyl 2-(tert-butyl) (1 S,3S,5R)-5-((3-hydroxypropoxy)methyl)-2-azabicyclo[3.1.0]hexane-2,3-dicarboxylate (130 mg, yield 53.8%) as a colorless oil. LCMS (ESI) m/z: 336 (M+H)⁺.

### Step 2: 3-benzyl 2-(tert-butyl) (1S,3S,5R)-5-((3-((methylsulfonyl)oxy)propoxy)methyl)-2-azabicyclo[3.1.0]hexane-2,3-dicarboxylate

To a solution of 3-benzyl 2-(tert-butyl) (1S,3S,5R)-5-((3-hydroxypropoxy)methyl)-2-azabicyclo[3.1.0]hexane-2,3-dicarboxylate (130 mg, 0.32 mmol) and TEA (130 mg, 1.0 mmol) in THE (2 mL) added MsCl (73 mg, 0.64 mmol) at 0° C. under N₂ atmosphere and the reaction mixture was stirred at room temperature for 16 hours. The mixture was diluted with EtOAc, washed with water and brine, dried over anhydrous Na₂SO₄, filtered, and concentrated under reduced pressure to dryness to give 3-benzyl 2-(tert-butyl) (1S,3S,5R)-5-((3-((methylsulfonyl)oxy)propoxy)methyl)-2-azabicyclo[3.1.0]hexane-2,3-dicarboxylate (150 mg, yield 96.6%) as a yellow solid, which was used directly in the next reaction without further purification. LCMS (ESI) m/z: 484 (M+H)⁺.

### Step 3: 3-benzyl 2-(tert-butyl) (1S,3S,5R)-5-((3-azidopropoxy)methyl)-2-azabicyclo [3.1.0]hexane-2,3-dicarboxylate

To a solution of 3-benzyl 2-(tert-butyl) (1 S,3S,5R)-5-((3-((methylsulfonyl) oxy)propoxy)methyl)-2-azabicyclo[3.1.0]hexane-2,3-dicarboxylate (150 mg, 0.31 mmol) in DMF (1.5 mL) was added NaN₃ (80 mg, 1.24 mmol) and the mixture was stirred at 60° C. overnight. The reaction mixture was diluted water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give 3-benzyl 2-(tert-butyl) (1 S,3S,5R)-5-((3-azidopropoxy)methyl)-2-azabicyclo[3.1.0]hexane-2,3-dicarboxylate (120 mg, yield 89.9%) as a colorless oil. LCMS (ESI) m/z: 431 (M+H)⁺.

### Step 4: benzyl (1S,3S,5R)-5-((3-azidopropoxy)methyl)-2-azabicyclo[3.1.0]hexane-3-carboxylate hydrochloride

A solution of 3-benzyl 2-(tert-butyl) (1S,3S,5R)-5-((3-azidopropoxy)methyl)-2-azabicyclo[3.1.0]hexane-2,3-dicarboxylate (120 mg, 0.28 mmol) in HCl/1,4-dioxane (2 mL) was stirred under N₂ atmosphere at room temperature for 2 hours. The reaction mixture was concentrated under reduced pressure to dryness, co-evaporated with DCM twice and dried under vacuum to give benzyl (1S,3S,5R)-5-((3-azidopropoxy)methyl)-2-azabicyclo[3.1.0]hexane-3-carboxylate hydrochloride (100 mg, yield 97.5%) as a yellow solid, which was used directly in the next reaction without further purification. LCMS (ESI) m/z: 331 (M+H)⁺.

### Step 5: benzyl (1S,3S,5R)-5-((3-azidopropoxy)methyl)-2-((9,9-difluoro-9H-fluorene-3-carbonyl)glycyl)-2-azabicyclo[3.1.0]hexane-3-carboxylate

To a mixture of benzyl (1 S,3S,5R)-5-((3-azidopropoxy)methyl)-2-azabicyclo [3.1.0]hexane-3-carboxylate hydrochloride (180 mg, 0.54 mmol) and (9,9-difluoro-9H-fluorene-3-carbonyl)glycine (160 mg, 0.53 mmol) in DMF (2 mL) was added DIPEA (260 mg, 2.0 mmol) and T₃P (504 mg, 0.79 mmol, 50% wt. in EtOAc) under N₂ atmosphere and the mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-80% EtOAc in PE) to give benzyl (1 S,3S,5R)-5-((3-azidopropoxy)methyl)-2-((9,9-difluoro-9H-fluorene-3-carbonyl) glycyl)-2-azabicyclo[3.1.0]hexane-3-carboxylate (190 mg, yield 95.2%) as a yellow oil. LC/MS (ESI) m/z: 616 (M+H)⁺.

### Step 6: (1S,3S,5R)-5-((3-azidopropoxy)methyl)-2-((9,9-difluoro-9H-fluorene-3-carbonyl)glycyl)-2-azabicyclo[3.1.0]hexane-3-carboxylic acid

To a solution of benzyl (1S,3S,5R)-5-((3-azidopropoxy)methyl)-2-((9,9-difluoro-9H-fluorene-3-carbonyl)glycyl)-2-azabicyclo[3.1.0]hexane-3-carboxylate (190 mg, 0.31 mmol) in MeOH (1 mL), THE (0.5 mL) and water (0.5 mL) was added LiOH·H₂O (42 mg, 1.0 mmol) at 0° C. and the mixture was stirred at room temperature for 4 hours. The reaction mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (1 S,3S,5R)-5-((3-azidopropoxy)methyl)-2-((9,9-difluoro-9H-fluorene-3-carbonyl)glycyl)-2-azabicyclo [3.1.0]hexane-3-carboxylic acid (160 mg, yield 98%) as a white solid, which was used in next reaction without purification. LC/MS (ESI) m/z: 526 (M+H)⁺.

### Step 7: (1S,3S,5R)-5-((3-azidopropoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide

To a mixture of (R)-5-(1-aminoethyl)thiophene-3-carboximidamide hydrochloride (85 mg, 0.41 mmol) and (1 S,3S,5R)-5-((3-azidopropoxy)methyl)-2-((9,9-difluoro-9H-fluorene-3-carbonyl)glycyl)-2-azabicyclo[3.1.0]hexane-3-carboxylic acid (180 mg, 0.34 mmol) in DMF (2 mL) was added DIPEA (153 mg, 1.19 mmol) and T₃P (226 mg, 0.36 mmol, 50% wt. in EtOAc) under N₂ atmosphere and the mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-20% MeOH in DCM) to give (1 S,3S,5R)-5-((3-azidopropoxy)methyl)-N—((R)-1-(4-carbamimidoylthiophen-2-yl)ethyl)-2-((9,9-difluoro-9H-fluorene-3-carbonyl) glycyl)-2-azabicyclo[3.1.0]hexane-3-carboxamide (230 mg, yield 94.3%) as a yellow oil. LC/MS (ESI) m/z: 677 (M+H)⁺.

### Step 8: tert-butyl ((5-((R)-1-((1S,3S,5R)-5-((3-azidopropoxy)methyl)-2-((9,9-difluoro-9H-fluorene-3-carbonyl)glycyl)-2-azabicyclo[3.1.0]hexane-3-carboxamido)ethyl) thiophen-3-yl)(imino)methyl)carbamate

To a solution of (1 S,3S,5R)-5-((3-azidopropoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (220 mg, 0.32 mmol) in THE (2.2 mL) and saturated aq·NaHCO₃ (2.2 mL) was added (Boc)₂O (210 mg, 0.96 mmol) and the mixture was stirred at room temperature overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-20% MeOH in DCM) to give tert-butyl ((5-((R)-1-((1S,3S,5R)-5-((3-azidopropoxy)methyl)-2-((9,9-difluoro-9H-fluorene-3-carbonyl)glycyl)-2-azabicyclo[3.1.0]hexane-3-carboxamido)ethyl)thiophen-3-yl)(imino) methyl)carbamate (90 mg, yield 35.0%) as a yellow oil. LC/MS (ESI) m/z: 777 (M+H)⁺.

### Step 9: tert-butyl ((5-((R)-1-((1S,3S,5R)-5-((3-aminopropoxy)methyl)-2-((9,9-difluoro-9H-fluorene-3-carbonyl)glycyl)-2-azabicyclo[3.1.0]hexane-3-carboxamido) ethyl)thiophen-3-yl)(imino)methyl)carbamate

To a mixture of tert-butyl ((5-((R)-1-((1S,3S,5R)-5-((3-azidopropoxy)methyl)-2-((9,9-difluoro-9H-fluorene-3-carbonyl)glycyl)-2-azabicyclo[3.1.0]hexane-3-carboxamido) ethyl)thiophen-3-yl)(imino)methyl)carbamate (80 mg, 0.10 mmol) in THE (1 mL) and water (1 mL) was added PPh₃ (23 mg, 0.1 mmol) and the mixture was stirred at room temperature for 16 hours. The mixture concentrated to dryness and the residue was purified by prep-HPLC to give tert-butyl ((5-((R)-1-((1 S,3S,5R)-5-((3-aminopropoxy)methyl)-2-((9,9-difluoro-9H-fluorene-3-carbonyl)glycyl)-2-azabicyclo[3.1.0]hexane-3-carboxamido)ethyl)thiophen-3-yl)(imino) methyl)carbamate (22 mg, yield 28.5%) as a white solid. LC/MS (ESI) m/z: 751 (M+H)⁺.

### Step 10: (1S,3S,5R)-5-((3-aminopropoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 362)

A mixture of tert-butyl ((5-((R)-1-((1S,3S,5R)-5-((3-aminopropoxy)methyl)-2-((9,9-difluoro-9H-fluorene-3-carbonyl)glycyl)-2-azabicyclo[3.1.0]hexane-3-carboxamido) ethyl)thiophen-3-yl)(imino)methyl)carbamate (20 mg, 0.026 mmol) in HCl/1,4-dioxane (1 mL) was stirred under N₂ atmosphere at room temperature overnight. The reaction mixture was concentrated under reduced pressure to dryness. The residue was purified by prep-HPLC to give Compound 382 (1.5 mg, yield 8.6%) as a white solid. ¹H NMR (400 MHz, CD₃OD) δ 8.52 (s, 3H), 8.26-8.17 (m, 2H), 7.89 (dd, J=7.8, 1.4 Hz, 1H), 7.75 (dd, J=12.1, 7.7 Hz, 2H), 7.67 (d, J=7.4 Hz, 1H), 7.59 (t, J=7.6 Hz, 1H), 7.48 (dd, J=18.0, 10.4 Hz, 2H), 5.24 (q, J=6.5 Hz, 1H), 4.91-4.88 (m, 1H), 4.52 (d, J=16.5 Hz, 1H), 4.29 (d, J=16.5 Hz, 1H), 3.65-3.58 (m, 4H), 3.53 (d, J=10.4 Hz, 1H), 3.07 (t, J=7.2 Hz, 2H), 2.70 (t, J=12.4 Hz, 1H), 2.18-2.11 (m, 1H), 1.96 (dt, J=12.8, 6.2 Hz, 2H), 1.59 (d, J=7.0 Hz, 3H), 1.40 (dd, J=5.8, 2.6 Hz, 1H), 1.04 (t, J=5.9 Hz, 1H). LCMS (ESI) (m/z): 651 (M+H)⁺.

### Scheme 362: Synthesis of N-(2-((2S,4R)-2-(((R)-1-(4-carbamimidoylthiophen-2-yl)-2-hydroxyethyl)carbamoyl)-4-(methylsulfonyl)pyrrolidin-1-yl)-2-oxoethyl)-3-fluoro-5-phenylpicolinamide (Compound 383)

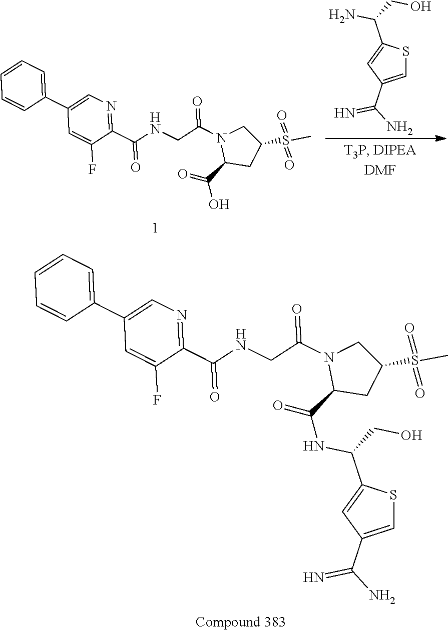 [SMILES: CS(=O)(=O)[C@@H]1C[C@@H](C(=O)O)N(C(=O)CNC(=O)c2ncc(-c3ccccc3)cc2F)C1 ; N=C(N)c1csc([C@H](N)CO)c1 ; CS(=O)(=O)[C@@H]1C[C@@H](C(=O)N[C@H](CO)c2cc(C(=N)N)cs2)N(C(=O)CNC(=O)c2ncc(-c3ccccc3)cc2F)C1]

Compound 383 was prepared from (2S,4R)-1-((3-fluoro-5-phenylpicolinoyl)glycyl)-4-(methylsulfonyl)pyrrolidine-2-carboxylic acid and (R)-5-(1-amino-2-hydroxyethyl)thiophene-3-carboximidamide based on the procedures set forth in Scheme 66. ¹H NMR (400 MHz, CD₃OD) δ 8.79 (d, J=7.4 Hz, 1H), 8.54 (s, 1H), 8.25 (dd, J=8.7, 7.1 Hz, 1H), 8.00 (t, J=10.3 Hz, 1H), 7.77 (d, J=5.4 Hz, 2H), 7.55 (dt, J=13.5, 7.1 Hz, 4H), 5.28 (dt, J=48.8, 5.8 Hz, 1H), 4.80 (dd, J=8.5, 4.5 Hz, 1H), 4.33 (d, J=5.2 Hz, 1H), 4.17 (dd, J=11.4, 7.0 Hz, 3H), 4.10-3.82 (m, 3H), 3.07 (d, J=17.6 Hz, 3H), 2.92-2.73 (m, 1H), 2.63-2.45 (m, 1H). LC/MS (ESI) m/z: 617 (M+H)⁺.

### Scheme 363: Synthesis of N-(2-((2S,4R)-2-(((R)-1-(4-carbamimidoylthiophen-2-yl)ethyl)carbamoyl)-4-fluoro-4-(methoxymethyl)pyrrolidin-1-yl)-2-oxoethyl)-9H-carbazole-3-carboxamide (Compound 389)

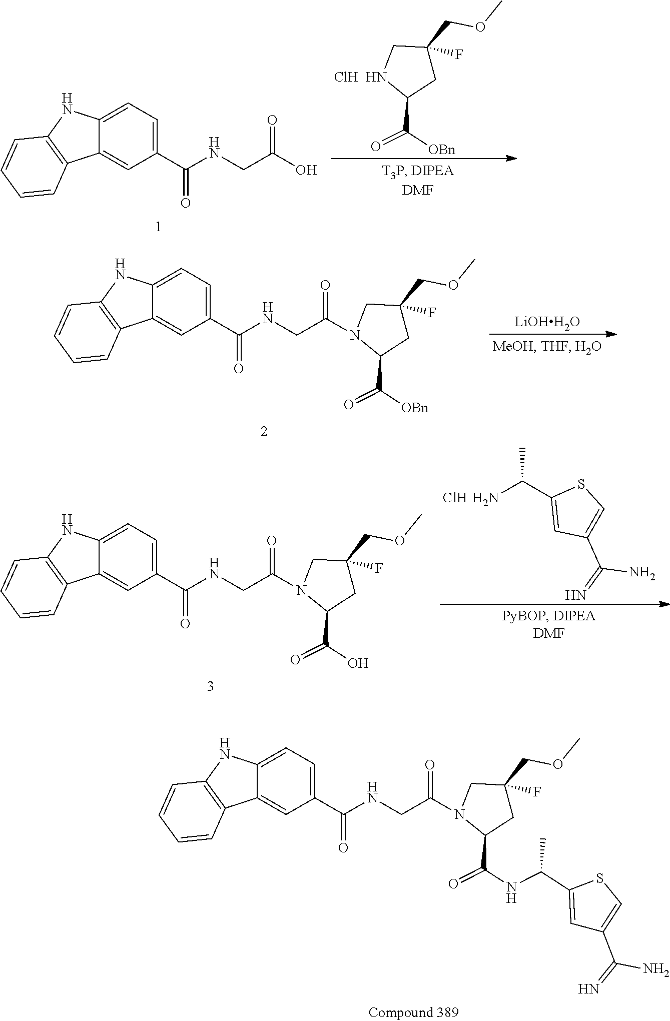

Compound 389 was prepared from (9H-carbazole-3-carbonyl)glycine, benzyl (2S,4R)-4-fluoro-4-(methoxymethyl)pyrrolidine-2-carboxylate hydrochloride, and (R)-5-(1-aminoethyl)thiophene-3-carboximidamide hydrochloride based on the procedures set forth in Scheme 3. ¹H NMR (400 MHz, CD₃OD) δ 8.65 (s, 1H), 8.53 (s, 1H), 8.22 (d, J=25.0 Hz, 1H), 8.11 (t, J=11.4 Hz, 1H), 7.91 (t, J=8.6 Hz, 1H), 7.58 (d, J=10.7 Hz, 1H), 7.49 (d, J=7.8 Hz, 2H), 7.43 (t, J=7.6 Hz, 1H), 7.22 (t, J=7.4 Hz, 1H), 5.31 (dt, J=21.6, 6.6 Hz, 1H), 4.65 (t, J=8.6 Hz, 1H), 4.35 (d, J=16.4 Hz, 1H), 4.22-4.06 (m, 2H), 3.99-3.86 (m, 1H), 3.75-3.66 (m, 2H), 3.44 (s, 3H), 2.56 (td, J=15.1, 7.6 Hz, 1H), 2.20 (ddd, J=23.4, 14.3, 9.7 Hz, 1H), 1.63 (dd, J=33.6, 6.8 Hz, 3H). LC/MS (ESI) m/z: 579 (M+H)⁺.

### Scheme 364: Synthesis of (2S,4R)—N—((R)-1-(4-carbamimidoylthiophen-2-yl)ethyl)-1-((9-methyl-9H-fluorene-2-carbonyl)glycyl)-4-(methylsulfonyl)pyrrolidine-2-carboxamide (Compound 390)

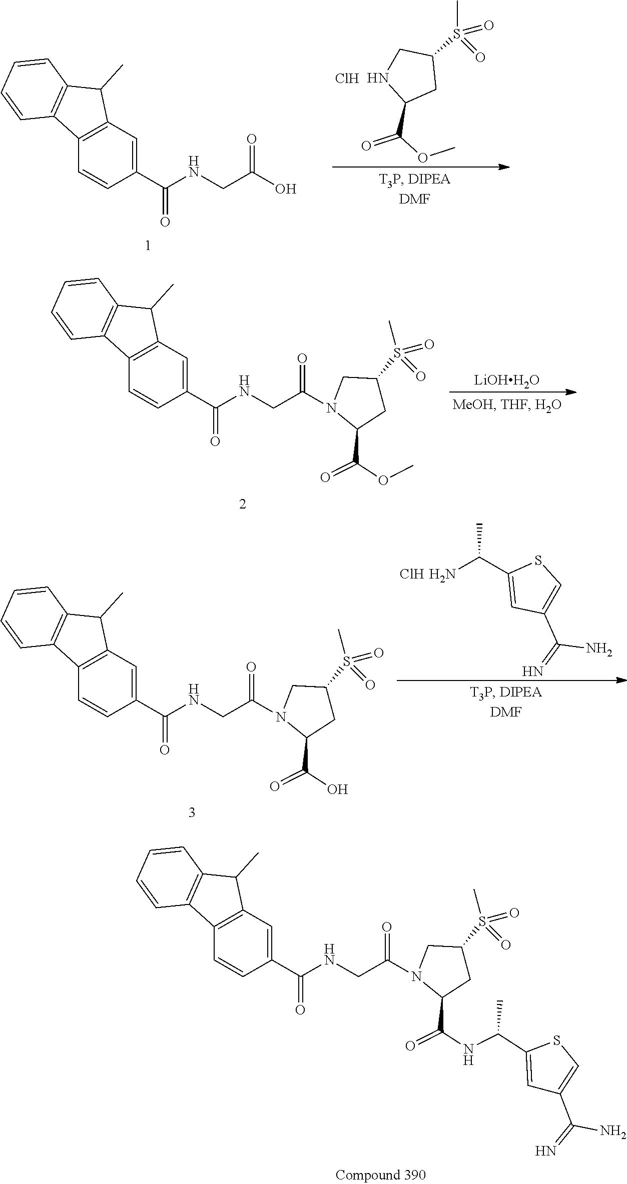

Compound 390 was prepared from a mixture of (9-methyl-9H-fluorene-2-carbonyl)glycine, methyl (2S,4R)-4-(methylsulfonyl)pyrrolidine-2-carboxylate hydrochloride, and (R)-5-(1-aminoethyl)thiophene-3-carboximidamide hydrochloride based on the procedures set forth in Scheme 66. ¹H NMR (400 MHz, CD₃OD) δ 8.51 (s, 1H), 8.23 (d, J=15.1 Hz, 1H), 8.04 (d, J=9.0 Hz, 1H), 7.86 (dd, J=11.4, 4.0 Hz, 3H), 7.60-7.50 (m, 2H), 7.41-7.37 (m, 2H), 5.40-5.25 (m, 1H), 4.80-4.71 (m, 1H), 4.30 (s, 1H), 4.24-3.94 (m, 5H), 3.07 (d, J=18.4 Hz, 3H), 2.93-2.74 (m, 1H), 2.50 (ddd, J=18.7, 11.4, 5.1 Hz, 1H), 1.64 (dd, J=31.4, 7.0 Hz, 3H), 1.55-1.52 (m, 3H). LCMS (ESI) (m/z): 608 (M+H)⁺.

### Scheme 365: Synthesis of (S)-7-((acridine-2-carbonyl)glycyl)-N—((R)-1-(4-carbamimidoylthiophen-2-yl)ethyl)-1,4-dioxa-7-azaspiro[4.4]nonane-8-carboxamide (Compound 391)

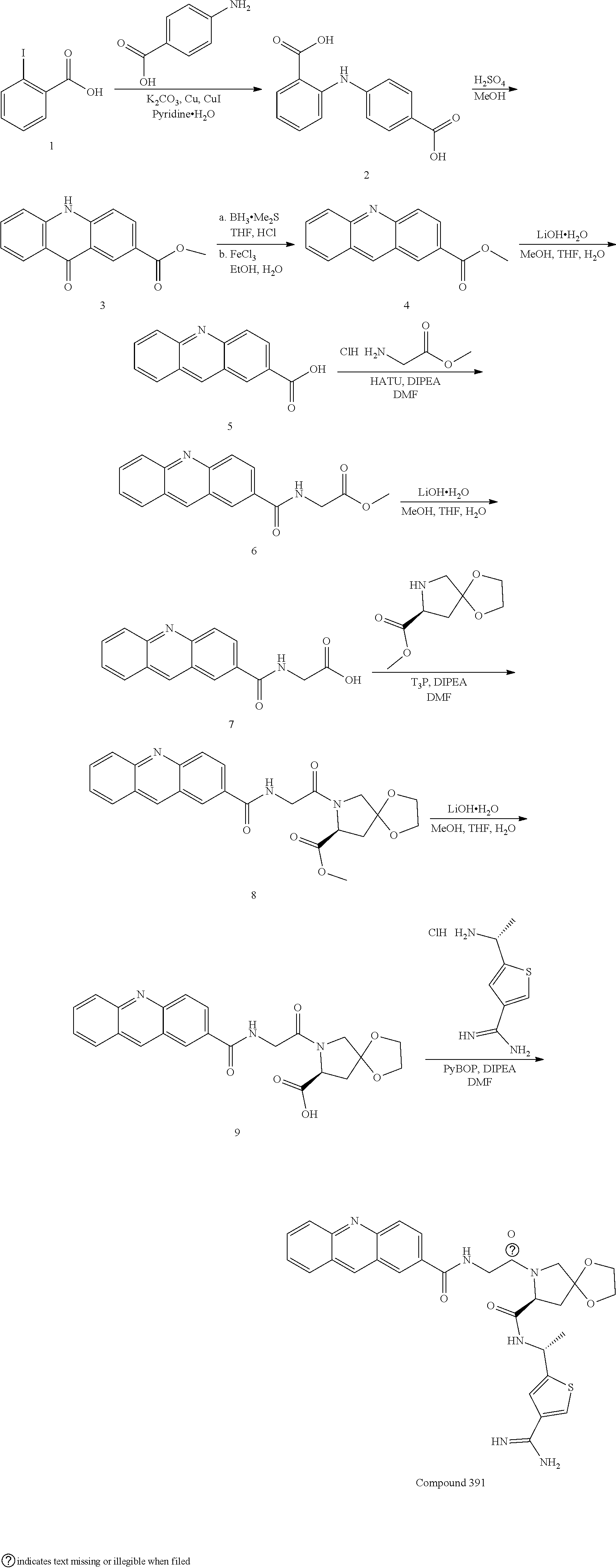

### Step 1: 2-((4-carboxyphenyl)amino)benzoic acid (2)

To a solution of K₂CO₃ (2.5 g, 18.1 mmol) in H₂O (70 mL) was added 2-iodobenzoic acid (3.0 g, 12.1 mmol), 4-aminobenzoic acid (3.3 g, 24.0 mmol), Pyridine (0.2 mL), Cu (70 mg) and CuI (70 mg) and the mixture was stirred at 100° C. for 48 hours. The mixture was acidified with 1 N aq·HCl to pH ˜1 and extracted with EtOAc twice. The combined organic layers were dried over anhydrous Na₂SO₄, filtered, and concentrated to give 2-((4-carboxyphenyl)amino) benzoic acid (1.2 g, yield 38.5%) as a yellow solid, which was used directly in the next reaction without further purification. LC/MS (ESI) (m/z): 258 (M+H)⁺.

### Step 2: methyl 9-oxo-9,10-dihydroacridine-2-carboxylate (3)

To a solution of 2-((4-carboxyphenyl)amino)benzoic acid (1.2 g, 4.67 mmol) in MeOH (10 mL) was added H₂SO₄ (0.1 mL) and the mixture was stirred at 80° C. for 16 hours. The mixture was basified with aq·NaHCO₃ to pH ˜9 and extracted with EtOAc twice. The combined organic layers were dried over anhydrous Na₂SO₄, filtered, and concentrated to dryness. The residue was purified by flash chromatography (silica gel, 0-10% MeOH in DCM) to give methyl 9-oxo-9,10-dihydroacridine-2-carboxylate (563 mg, yield 47.7%) as a yellow solid. LC/MS (ESI) (m/z): 254 (M+H)⁺.

### Step 3: methyl acridine-2-carboxylate (4)

To a solution of methyl 9-oxo-9,10-dihydroacridine-2-carboxylate (100 mg, 0.40 mmol) in THF (6 mL) was added BH₃·Me₂S (0.08 mL, 0.80 mmol, 10 M) at 0° C., and the mixture was stirred at 40° C. for 16 hours. The mixture was quenched with 1 N aq·HCl at 0° C. and extracted with DCM twice. The combined organic layers were dried over anhydrous Na₂SO₄, filtered, and concentrated to dryness. The residue was diluted with a solution of FeCl₃ (65 mg, 0.40 mmol) in EtOH (5 mL)/H₂O (1 mL) and the resulting mixture was stirred at 50° C. for 1 hour. The mixture was basified with aq·NaHCO₃ to pH ˜9 and extracted with EtOAc twice. The combined organic layers were dried with anhydrous Na₂SO₄, filtered, and concentrated under reduced pressure to dryness to give methyl acridine-2-carboxylate (80 mg, yield 85.4%) as a brown oil, which was used directly in the next reaction. LC/MS (ESI) (m/z): 238 (M+H)⁺.

### Step 4: acridine-2-carboxylic acid (5)

To a solution of methyl acridine-2-carboxylate (80 mg, 0.34 mmol) in MeOH/THF/H₂O (2 mL, v/v/v=2/1/1) was added LiOH·H₂O (42 mg, 1.01 mmol) and the mixture was stirred at room temperature for 2 hours. The mixture was acidified with 1 N aq·HCl to pH ˜3 and concentrated under reduced pressure to dryness to give acridine-2-carboxylic acid (75 mg, yield 99.7%) as a yellow solid, which was used directly in the next reaction. LCMS (ESI) (m/z): 224 (M+H)⁺.

### Step 5: methyl (acridine-2-carbonyl)glycinate (6)

To a mixture of acridine-2-carboxylic acid (75 mg, 0.34 mmol) and methyl glycinate hydrochloride (50 mg, 0.40 mmol) in DMF (3 mL) was added HATU (191 mg, 0.50 mmol) and DIPEA (217 mg, 1.68 mmol) and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give methyl (acridine-2-carbonyl)glycinate (43 mg, yield 43.5%) as a colorless oil. LC/MS (ESI) m/z: 295 (M+H)⁺.

### Step 6: (acridine-2-carbonyl)glycine (7)

To a solution of methyl acridine-2-carboxylate (40 mg, 0.14 mmol) in MeOH/THF/H₂O (2 mL, v/v/v=2/1/1) was added LiOH·H₂O (17 mg, 0.41 mmol) and the mixture was stirred at room temperature for 2 hours. The mixture was acidified with 1 N aq·HCl to pH ˜3 and concentrated under reduced pressure to dryness to give (acridine-2-carbonyl)glycine (40 mg, yield 97.7%) as a yellow solid, which was used directly in the next reaction. LCMS (ESI) (m/z): 281 (M+H)⁺.

### Step 7: methyl (S)-7-((acridine-2-carbonyl)glycyl)-1,4-dioxa-7-azaspiro[4.4]nonane-8-carboxylate (8)

To a mixture of (acridine-2-carbonyl)glycine (40 mg, 0.14 mmol) and methyl (S)-1,4-dioxa-7-azaspiro[4.4]nonane-8-carboxylate (53 mg, 0.29 mmol) in DMF (3 mL) was added DIPEA (92 mg, 0.71 mmol) and T₃P (136 mg, 0.21 mmol, 50% in EtOAc wt.) under N₂ atmosphere at 0° C. and the mixture was stirred at room temperature for 1 hour. The mixture was quenched with saturated aq·NaHCO₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-5% MeOH in DCM) to give methyl (S)-7-((acridine-2-carbonyl)glycyl)-1,4-dioxa-7-azaspiro[4.4]nonane-8-carboxylate (42 mg, yield 65.6%) as a colorless oil. LCMS (ESI) (m/z): 450 (M+H)⁺.

### Step 6: (S)-7-((acridine-2-carbonyl)glycyl)-1,4-dioxa-7-azaspiro[4.4]nonane-6-carboxylic acid (9)

To a solution of methyl (S)-7-((acridine-2-carbonyl)glycyl)-1,4-dioxa-7-azaspiro[4.4]nonane-8-carboxylate (40 mg, 0.09 mmol) in MeOH/THF/H₂O (2 mL, v/v/v=2/1/1) was added LiOH—H₂O (11 mg, 0.27 mmol) and the mixture was stirred at room temperature for 2 hours. The mixture was acidified with 1 N aq·HCl to pH˜3 and concentrated under reduced pressure to dryness to give (S)-7-((acridine-2-carbonyl)glycyl)-1,4-dioxa-7-azaspiro[4.4]nonane-8-carboxylic acid (30 mg, yield 77.3%) as a yellow solid, which was used directly in the next reaction. LCMS (ESI) (m/z): 436 (M+H)⁺.

### Step 9: (S)-7-((acridine-2-carbonyl)glycyl)-N—((R)-1-(4-carbamimidoylthiophen-2-yl)ethyl)-1,4-dioxa-7-azaspiro[4.4]nonane-6-carboxamide (Compound 391)

To a mixture of (S)-7-((acridine-2-carbonyl)glycyl)-1,4-dioxa-7-azaspiro[4.4]nonane-8-carboxylic acid (30 mg, 0.07 mmol) and (R)-5-(1-aminoethyl)thiophene-3-carboximidamide hydrochloride (28 mg, 0.14 mmol) in DMF (2 mL) was added DIPEA (44 mg, 0.34 mmol) and PyBop (54 mg, 0.10 mmol) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=8:1) and further purified by prep-HPLC to give Compound 391 (0.2 mg, yield 0.5%) as a white solid. 1H NMR (400 MHz, CD₃OD) δ 9.21 (s, 1H), 8.72 (t, J=2.0 Hz, 1H), 8.46 (t, J=3.8 Hz, 1H), 8.25-8.22 (m, 4H), 8.20 (s, 1H), 7.97-7.93 (m, 1H), 7.71-7.66 (m, 1H), 7.53 (t, J=1.3 Hz, 1H), 5.29 (dd, J=7.1, 1.3 Hz, 1H), 4.58 (dd, J=8.4, 1.8 Hz, 1H), 4.27 (d, J=5.2 Hz, 1H), 4.05-3.97 (m, 5H), 3.82 (t, J=9.0 Hz, 2H), 2.51-2.38 (m, 1H), 2.23 (dd, J=13.2, 6.1 Hz, 1H), 1.64 (d, J=34.3 Hz, 3H).LC/MS (ESI) m/z: 587 (M+H)⁺.

### Scheme 366: Synthesis of (2S,4R)—N—((R)-1-(4-carbamimidoylthiophen-2-yl)ethyl)-1-((9-(difluoromethoxy)-9H-fluorene-3-carbonyl)glycyl)-4-(methylsulfonyl)pyrrolidine-2-carboxamide (Compound 392)

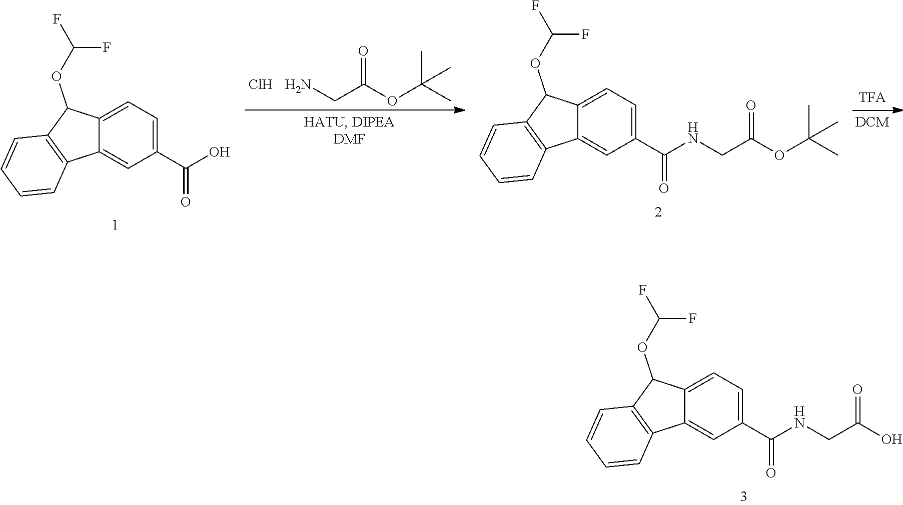

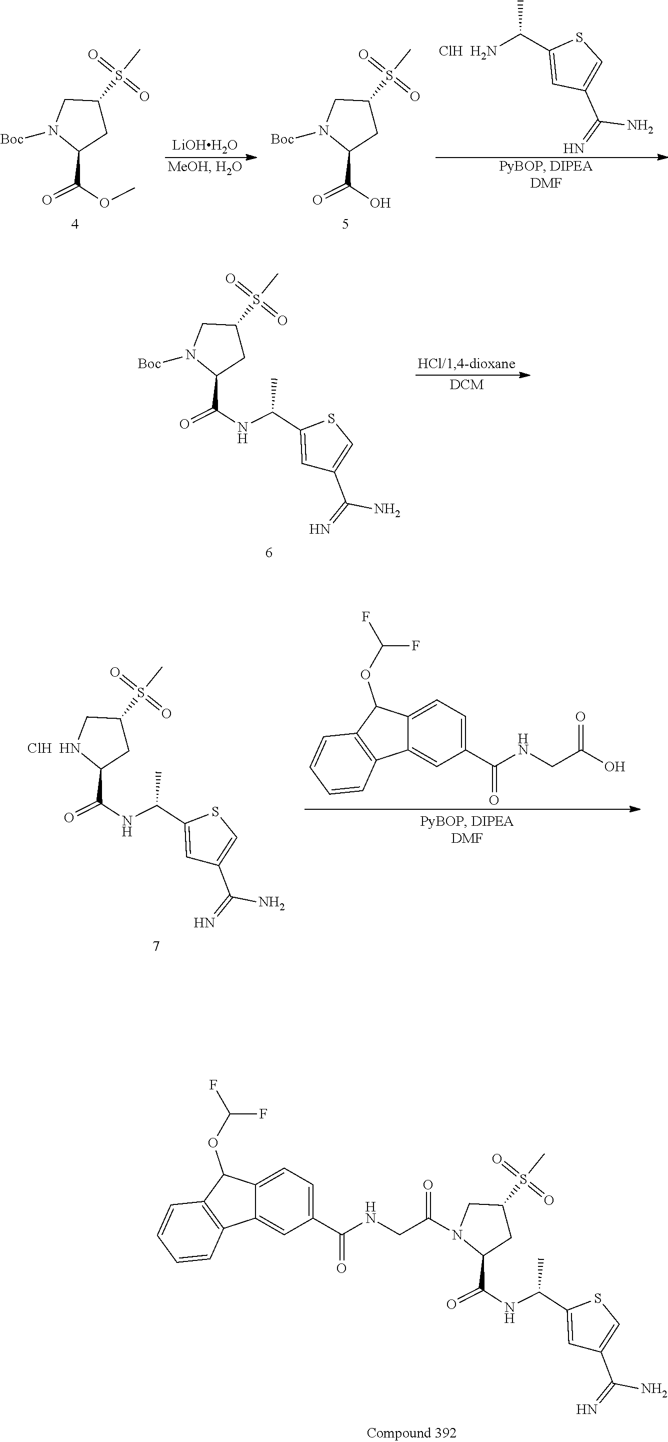

### Step 1: tert-butyl (9-(difluoromethoxy)-9H-fluorene-3-carbonyl)glycinate

To a mixture of 9-(difluoromethoxy)-9H-fluorene-3-carboxylic acid (30 mg, 0.11 mmol) and tert-butyl glycinate hydrochloride (22 mg, 0.13 mmol) in DMF (3 mL) was added DIPEA (70 mg, 0.54 mmol) and HATU (57 mg, 0.15 mmol) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-25% EtOAc in PE) to give tert-butyl (9-(difluoromethoxy)-9H-fluorene-3-carbonyl)glycinate (32 mg, yield 76.2%) as a light-yellow oil. LCMS (ESI) m/z: 390 (M+H)⁺.

### Step 2: (9-(difluoromethoxy)-9H-fluorene-3-carbonyl)glycine

To a solution of tert-butyl (9-(difluoromethoxy)-9H-fluorene-3-carbonyl)glycinate (32 mg, 0.065 mmol) in DCM (1 mL) was added TFA (1 mL) and the mixture was stirred under N₂ atmosphere at room temperature for 2 hours. The reaction mixture was concentrated under reduced pressure to dryness to give (9-(difluoromethoxy)-9H-fluorene-3-carbonyl)glycine (27 mg, yield 98.6%) as a yellow solid, which was used directly in the next step without further purification. LCMS (ESI) m/z: 334 (M+H)⁺.

### Step 3: (2S,4R)-1-(tert-butoxycarbonyl)-4-(methylsulfonyl)pyrrolidine-2-carboxylic acid

To a solution of 1-(tert-butyl) 2-methyl (2S,4R)-4-(methylsulfonyl)pyrrolidine-1,2-dicarboxylate (210 mg, 0.68 mmol) in THF/MeOH/H₂O (4 mL, v/v/v=1/2/1) was added LiOH·H₂O (115 mg, 2.74 mmol) and the mixture was stirred at room temperature for 1 hour. The mixture was acidified with 1 N aq·HCl to pH ˜3 and extracted with EtOAc twice. The combined organic layers were washed with brine, dried over anhydrous Na₂SO₄, filtered, and concentrated under reduced pressure to dryness to give (2S,4R)-1-(tert-butoxycarbonyl)-4-(methylsulfonyl)pyrrolidine-2-carboxylic acid (200 mg, yield 99.8%) as a light-yellow oil, which was used directly in the next step without further purification. LC/MS (ESI) m/z: 294 (M+H)⁺.

### Step 4: tert-butyl (2S,4R)-2-(((R)-1-(4-carbamimidoylthiophen-2-yl)ethyl)carbamoyl)-4-(methylsulfonyl)pyrrolidine-1-carboxylate

To a mixture of (2S,4R)-1-(tert-butoxycarbonyl)-4-(methylsulfonyl)pyrrolidine-2-carboxylic acid (100 mg, 0.34 mmol) and (R)-5-(1-aminoethyl)thiophene-3-carboximidamide hydrochloride (105 mg, 0.51 mmol) in DMF (5 mL) was added DIPEA (220 mg, 1.71 mmol) and PyBop (266 mg, 0.51 mmol) under N₂ atmosphere and the reaction mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=8:1) and to give tert-butyl (2S,4R)-2-(((R)-1-(4-carbamimidoylthiophen-2-yl)ethyl)carbamoyl)-4-(methylsulfonyl) pyrrolidine-1-carboxylate (140 mg, yield 92.4%) as a white solid. LC/MS (ESI) m/z: 445 (M+H)⁺.

### Step 5: (2S,4R)—N—((R)-1-(4-carbamimidoylthiophen-2-yl)ethyl)-4-(methylsulfonyl) pyrrolidine-2-carboxamide hydrochloride

To a solution of tert-butyl (2S,4R)-2-(((R)-1-(4-carbamimidoylthiophen-2-yl)ethyl)carbamoyl)-4-(methylsulfonyl)pyrrolidine-1-carboxylate (140 mg, 0.32 mmol) in DCM (1 mL) was added HCl/1,4-dioxane (3 mL, 4 M) and the mixture was stirred under N₂ atmosphere at room temperature for 2 hours. The reaction mixture was concentrated under reduced pressure to dryness to give (2S,4R)—N—((R)-1-(4-carbamimidoylthiophen-2-yl)ethyl)-4-(methylsulfonyl)pyrrolidine-2-carboxamide hydrochloride (100 mg, yield 92.2%) as a yellow solid, which was used directly in the next reaction without further purification. LCMS (ESI) m/z: 345 (M+H)⁺.

### Step 6: (2S,4R)—N—((R)-1-(4-carbamimidoylthiophen-2-yl)ethyl)-1-((9-(difluoromethoxy)-9H-fluorene-3-carbonyl)glycyl)-4-(methylsulfonyl)pyrrolidine-2-carboxamide (Compound 392)

To a mixture of (2S,4R)—N—((R)-1-(4-carbamimidoylthiophen-2-yl)ethyl)-4-(methylsulfonyl)pyrrolidine-2-carboxamide hydrochloride (37 mg, 0.097 mmol) and (9-(difluoromethoxy)-9H-fluorene-3-carbonyl)glycine (22 mg, 0.066 mmol) in DMF (2 mL) was added DIPEA (43 mg, 0.33 mmol) and PyBop (41 mg, 0.079 mmol) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=5:1) and further purified by prep-HPLC to give Compound 392 (0.5 mg, yield 1.1%) as a white solid. ¹H NMR (400 MHz, CD₃OD) δ 8.28-8.15 (m, 2H), 7.86-7.78 (m, 2H), 7.68 (d, J=7.6 Hz, 1H), 7.62 (d, J=7.7 Hz, 1H), 7.56-7.45 (m, 2H), 7.40 (t, J=6.9 Hz, 1H), 6.78 (t, J=73.9 Hz, 1H), 6.20 (s, 1H), 5.34-5.24 (m, 1H), 4.72 (dd, J=8.6, 4.7 Hz, 1H), 4.31-4.30 (m, 1H), 4.23-4.14 (m, 3H), 3.52-3.44 (m, 1H), 3.08 (d, J=7.0 Hz, 3H), 2.89-2.66 (m, 1H), 2.49-2.37 (m, 1H), 1.59 (d, J=7.0 Hz, 3H). LCMS (ESI) m/z: 660 (M+H)⁺.

### Scheme 367: Synthesis of (2S,4R)—N—((R)-1-(4-carbamimidoylthiophen-2-yl)ethyl)-1-((5,5-dioxidodibenzo[b,d]thiophene-2-carbonyl)glycyl)-4-fluoro-4-(methoxymethyl) pyrrolidine-2-carboxamide (Compound 393)

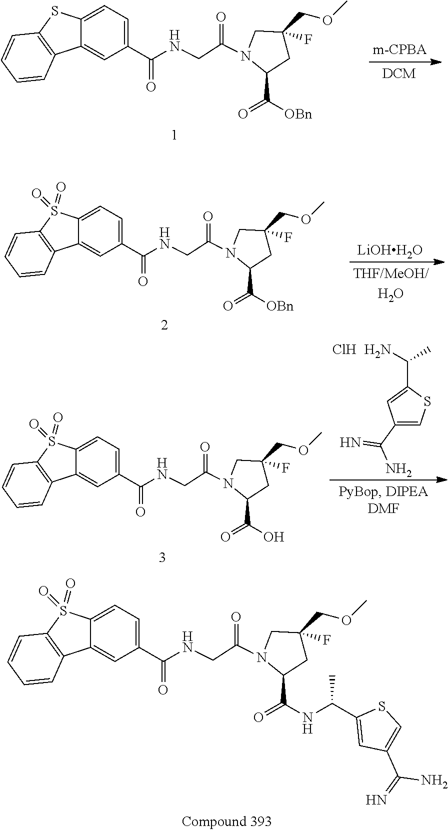

To a solution of benzyl (2S,4R)-1-((5,5-dioxidodibenzo[b,d]thiophene-2-carbonyl)glycyl)-4-fluoro-4-(methoxymethyl)pyrrolidine-2-carboxylate and (R)-5-(1-aminoethyl)thiophene-3-carboximidamide hydrochloride based on the procedures set forth in Scheme 251. ¹H NMR (400 MHz, CD₃OD) δ 8.50-8.47 (m, 2H), 8.26-8.18 (m, 1H), 8.08 (dd, J=13.2, 5.7 Hz, 2H), 7.96-7.93 (m, 1H), 7.90-7.87 (m, 1H), 7.82-7.78 (m, 1H), 7.70-7.66 (m, 1H), 7.57-7.52 (m, 1H), 5.31-5.24 (m, 1H), 4.66-4.60 (m, 1H), 4.34 (dd, J=47.5, 30.7 Hz, 2H), 4.11-4.03 (m, 1H), 3.98-3.87 (m, 1H), 3.74-3.66 (m, 2H), 3.45 (s, 3H), 2.56 (dddd, J=14.6, 7.8, 4.6, 1.5 Hz, 1H), 2.29-2.11 (m, 1H), 1.59 (d, J=7.0 Hz, 3H). LC/MS (ESI) m/z: 628 (M+H)⁺.

### Scheme 368: Synthesis of (2S,4R)—N—((R)-1-(4-carbamimidoylthiophen-2-yl)ethyl)-1-((dibenzo[b,d]thiophene-2-carbonyl)glycyl)-4-fluoro-4-(methoxymethyl)pyrrolidine-2-carboxamide (Compound 394)

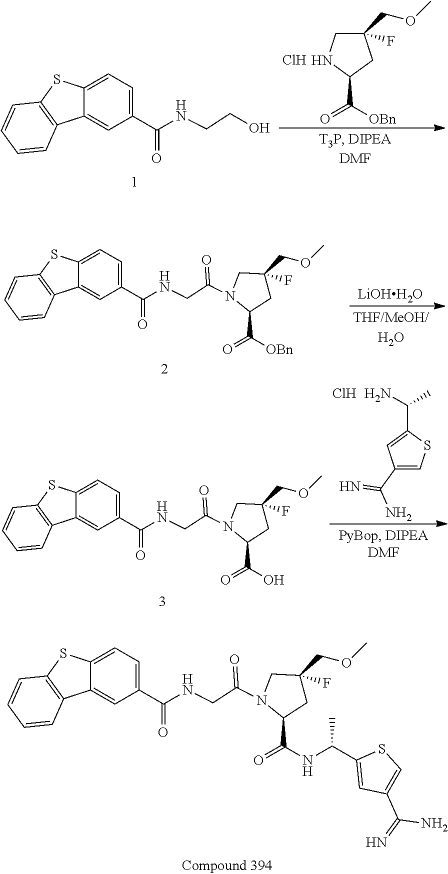

Compound 394 was prepared from (dibenzo[b,d]thiophene-2-carbonyl)glycine, benzyl (2S,4R)-4-fluoro-4-(methoxymethyl)pyrrolidine-2-carboxylate hydrochloride, and (R)-5-(1-amino-2-hydroxyethyl)thiophene-3-carboximidamide hydrochloride based on the procedures set forth in Scheme 3. ¹H NMR (400 MHz, CD₃OD) δ 8.76 (s, 1H), 8.54 (s, 1H), 8.29-8.12 (m, 2H), 7.97-7.91 (m, 3H), 7.54 (dt, J=4.1, 2.5 Hz, 3H), 5.36-5.24 (m, 1H), 4.68-4.61 (m, 1H), 4.40-4.16 (m, 2H), 4.09 (ddd, J=19.1, 12.2, 1.1 Hz, 1H), 4.00-3.88 (m, 1H), 3.72 (dd, J=17.6, 5.8 Hz, 2H), 3.44 (d, J=6.0 Hz, 3H), 2.56 (td, J=14.8, 7.7 Hz, 1H), 2.21 (ddd, J=37.0, 14.2, 9.6 Hz, 1H), 1.62 (dd, J=29.7, 6.1 Hz, 3H). LC/MS (ESI) m/z: 596 (M+H)⁺.

### Scheme 369: Synthesis of (2S,4R)—N—((R)-1-(4-carbamimidoylthiophen-2-yl)-2-hydroxyethyl)-1-((9,9-dimethyl-9H-fluorene-3-carbonyl)glycyl)-4-methoxypyrrolidine-2-carboxamide (Compound 395)

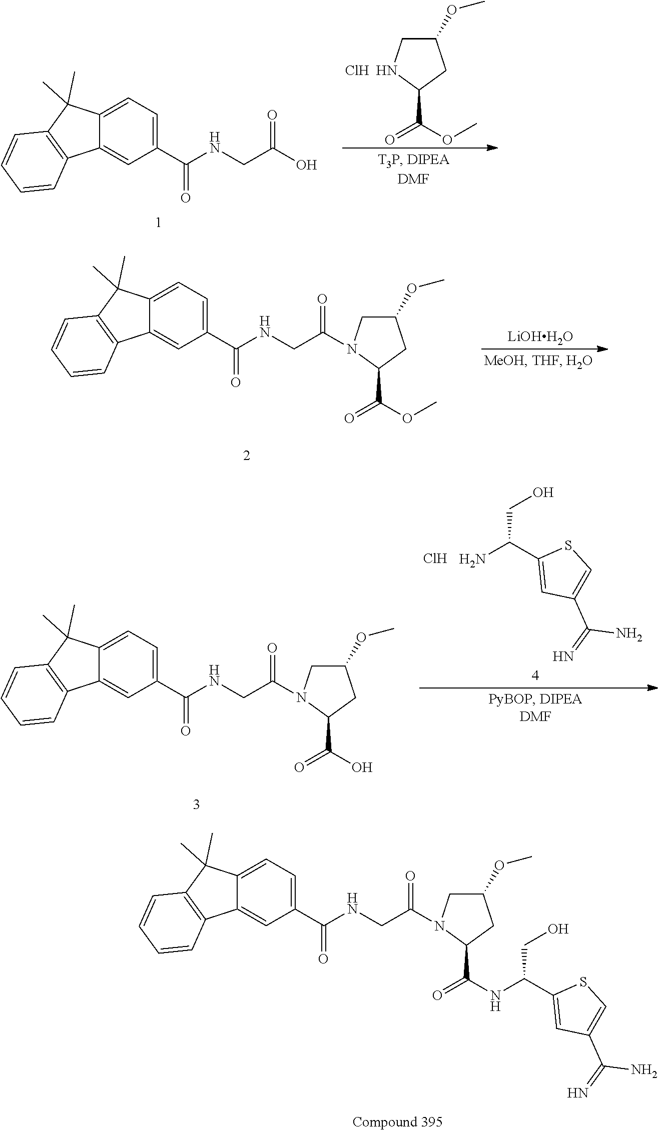

Compound 395 was prepared from (9,9-dimethyl-9H-fluorene-3-carbonyl)glycine, methyl (2S,4R)-4-methoxypyrrolidine-2-carboxylate hydrochloride, and (R)-5-(1-amino-2-hydroxyethyl)thiophene-3-carboximidamide hydrochloride based on the procedures set forth in Scheme 3. ¹H NMR (400 MHz, CD₃OD) δ 8.52 (s, 1H), 8.32-8.18 (m, 2H), 7.85-7.78 (m, 2H), 7.58 (d, J=8.2 Hz, 2H), 7.53-7.49 (m, 1H), 7.36 (dd, J=5.2, 3.4 Hz, 2H), 5.23 (t, J=5.8 Hz, 1H), 4.62-4.55 (m, 2H), 4.33 (d, J=16.8 Hz, 1H), 4.18 (s, 1H), 3.96-3.80 (m, 4H), 3.40 (s, 3H), 2.43 (dd, J=10.4, 2.9 Hz, 1H), 2.10 (ddd, 1H), 1.50 (s, 6H). LC/MS (ESI) m/z: 590 (M+H)⁺.

### Scheme 370: Synthesis of (2S,4R)—N—((R)-1-(4-carbamimidoylthiophen-2-yl)-2-hydroxyethyl)-1-((9,9-difluoro-9H-fluorene-2-carbonyl)glycyl)-4-(difluoromethoxy) pyrrolidine-2-carboxamide (Compound 396)

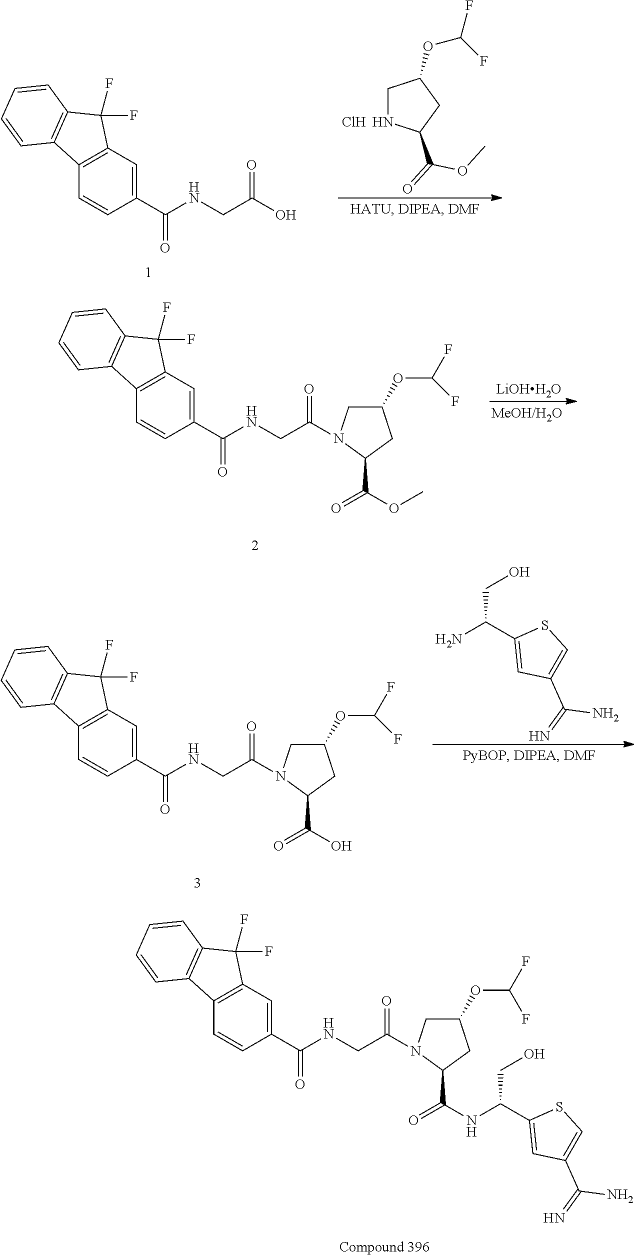

Compound 396 was prepared from (9,9-difluoro-9H-fluorene-2-carbonyl)glycine, methyl (2S,4R)-4-(difluoromethoxy)pyrrolidine-2-carboxylate hydrochloride, and (R)-5-(1-amino-2-hydroxyethyl)thiophene-3-carboximidamide based on the procedures set forth in Scheme 3. ¹H NMR (400 MHz, CD₃OD) δ 8.53 (s, 1H), 8.25 (dd, J=13.1, 1.6 Hz, 1H), 8.13-8.04 (m, 2H), 7.81 (dd, J=10.7, 7.9 Hz, 2H), 7.66 (d, J=7.6 Hz, 1H), 7.61-7.57 (m, 2H), 7.47 (t, J=7.5 Hz, 1H), 6.53 (t, J=74.4 Hz, 1H), 5.26 (dd, J=27.8, 22.0 Hz, 1H), 5.04 (s, 1H), 4.66 (t, J=7.8 Hz, 1H), 4.31 (d, J=16.7 Hz, 1H), 4.18 (d, J=16.7 Hz, 1H), 3.99-3.82 (m, 4H), 2.50 (ddd, J=19.5, 11.4, 7.1 Hz, 1H), 2.28 (s, 1H). LCMS (ESI) m/z=634 (M+H)⁺.

### Scheme 371: Synthesis of (2S,4R)—N—((R)-1-(4-carbamimidoylthiophen-2-yl)-2-hydroxyethyl)-1-((9,9-difluoro-9H-fluorene-2-carbonyl)glycyl)-4-fluoro-4-(methoxymethyl)pyrrolidine-2-carboxamide (Compound 397)

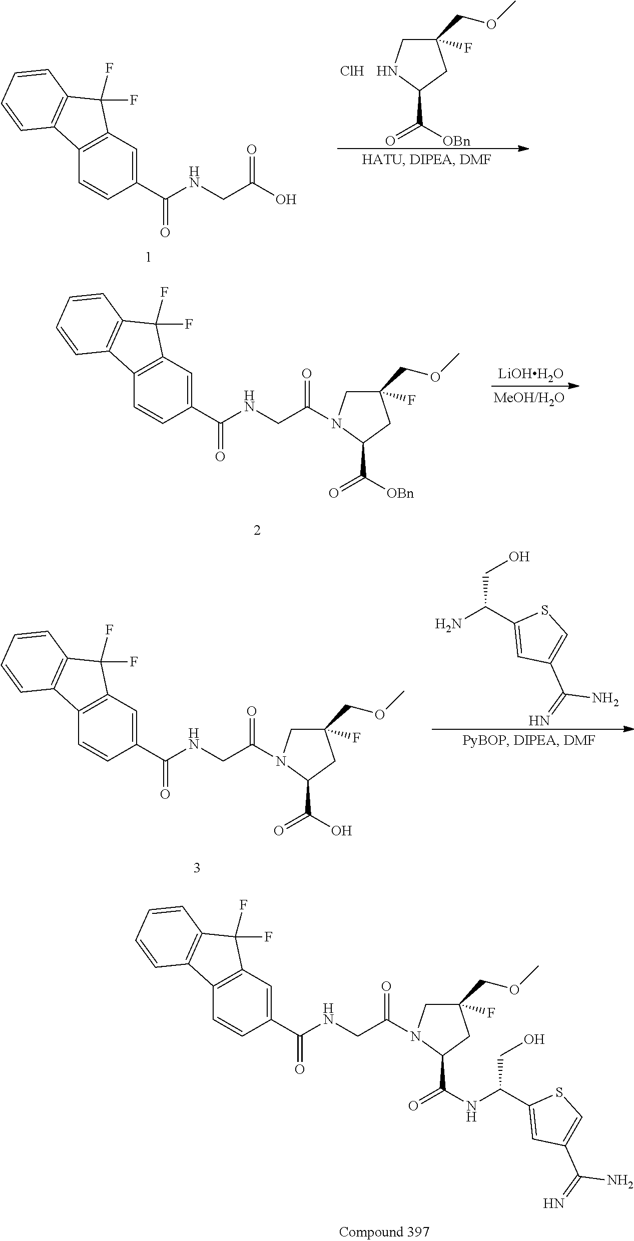

Compound 397 was prepared from (9,9-difluoro-9H-fluorene-2-carbonyl)glycine, benzyl (2S,4R)-4-fluoro-4-(methoxymethyl)pyrrolidine-2-carboxylate hydrochloride, and (R)-5-(1-amino-2-hydroxyethyl)thiophene-3-carboximidamide based on the procedures set forth in Scheme 3. ¹H NMR (400 MHz, CD₃OD) δ 8.53 (s, 1H), 8.25 (dd, J=14.3, 1.6 Hz, 1H), 8.12-8.04 (m, 2H), 7.83-7.78 (m, 2H), 7.66 (d, J=7.3 Hz, 1H), 7.60-7.56 (m, 2H), 7.47 (t, J=7.2 Hz, 1H), 5.23 (t, J=5.7 Hz, 1H), 4.72-4.65 (m, 1H), 4.33 (d, J=16.6 Hz, 1H), 4.17-4.02 (m, 2H), 3.96-3.81 (m, 3H), 3.74-3.66 (m, 2H), 3.43 (d, J=7.5 Hz, 3H), 2.57 (td, J=14.8, 7.9 Hz, 1H), 2.30-2.15 (m, 1H). LCMS (ESI) m/z=630 (M+H)⁺.

### Scheme 372: Synthesis of (2S,4R)—N—((R)-1-(4-carbamimidoylthiophen-2-yl)-2-hydroxyethyl)-1-((9,9-difluoro-9H-fluorene-3-carbonyl)glycyl)-4-fluoro-4-(fluoromethyl)pyrrolidine-2-carboxamide (Compound 398)

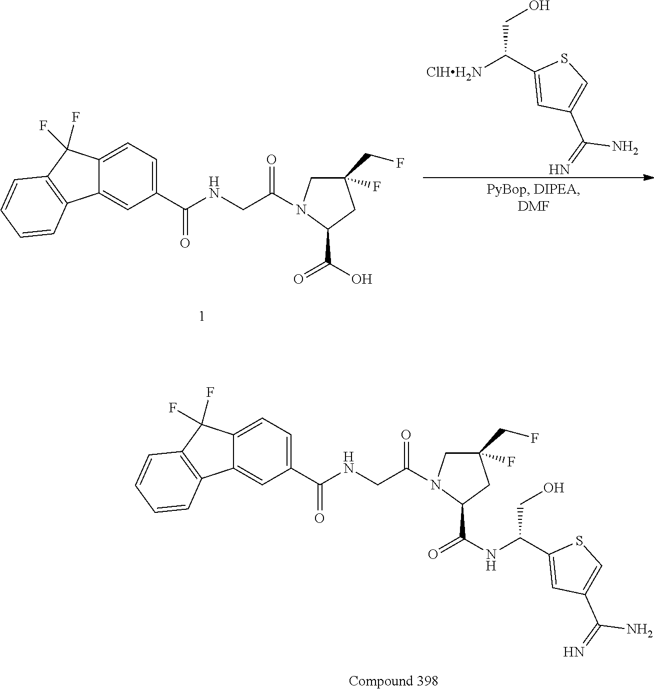

Compound 398 was prepared from (2S,4R)-1-((9,9-difluoro-9H-fluorene-3-carbonyl)glycyl)-4-fluoro-4-(fluoromethyl)pyrrolidine-2-carboxylic acid and (R)-5-(1-amino-2-hydroxyethyl)thiophene-3-carboximidamide hydrochloride based on the procedures set forth in Scheme 3. ¹H NMR (400 MHz, CD₃OD) δ 8.49 (s, 1H), 8.29-8.13 (m, 2H), 7.87 (dt, J=8.2, 4.1 Hz, 1H), 7.80-7.69 (m, 2H), 7.65 (d, J=7.3 Hz, 1H), 7.61-7.53 (m, 2H), 7.45 (t, J=7.4 Hz, 1H), 5.29 (dt, J=44.6, 5.8 Hz, 1H), 4.83-4.63 (m, 3H), 4.37 (d, J=16.8 Hz, 1H), 4.22-4.06 (m, 2H), 4.04-3.79 (m, 3H), 2.87-2.58 (m, 1H), 2.26 (ddd, J=34.9, 14.3, 9.1 Hz, 1H). LC/MS (ESI) m/z: 618 (M+H)⁺.

### Scheme 373: Synthesis of (S)—N—((R)-1-(4-carbamimidoylthiophen-2-yl)-2-hydroxyethyl)-7-((9,9-dimethyl-9H-fluorene-3-carbonyl)glycyl)-1,4-dioxa-7-azaspiro[4.4]nonane-8-carboxamide (Compound 399)

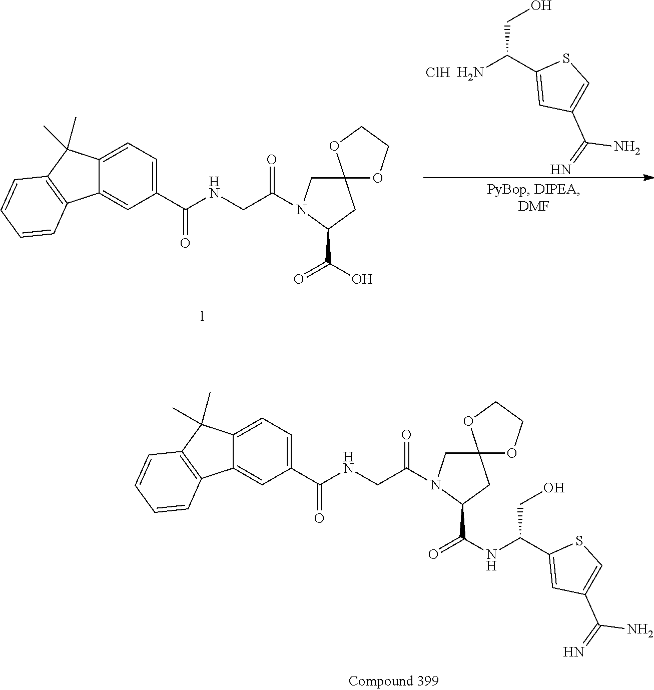 [SMILES: CC1(C)c2ccccc2-c2cc(C(=O)NCC(=O)N3CC4(C[C@H]3C(=O)O)OCCO4)ccc21 ; Cl ; N=C(N)c1csc([C@H](N)CO)c1 ; CC1(C)c2ccccc2-c2cc(C(=O)NCC(=O)N3CC4(C[C@H]3C(=O)N[C@H](CO)c3cc(C(=N)N)cs3)OCCO4)ccc21]

Compound 399 was prepared from (S)—N-(2-(8-(dihydroxymethyl)-1,4-dioxa-7-azaspiro[4.4]nonan-7-yl)-2-oxoethyl)-9,9-dimethyl-9H-fluorene-3-carboxamide and (R)-5-(1-amino-2-hydroxyethyl)thiophene-3-carboximidamide hydrochloride based on the procedures set forth in Scheme 3. ¹H NMR (400 MHz, CD₃OD) δ 8.47 (s, 1H), 8.27-8.12 (m, 2H), 7.83-7.75 (m, 2H), 7.57 (dd, J=12.8, 7.5 Hz, 2H), 7.51 (dd, J=5.8, 2.7 Hz, 1H), 7.42-7.35 (m, 2H), 5.39-5.17 (m, 1H), 4.64 (dd, J=8.9, 6.6 Hz, 1H), 4.21 (s, 1H), 4.04-3.90 (m, 6H), 3.86-3.82 (m, 3H), 2.46 (dd, J=13.2, 9.0 Hz, 1H), 2.30-2.21 (m, 1H), 1.50 (s, 6H). LC/MS (ESI) m/z: 618 (M+H)⁺.

### Scheme 374: Synthesis of (2S,4R)—N—((R)-1-(4-carbamimidoylthiophen-2-yl)-2-hydroxyethyl)-1-((9,9-difluoro-9H-fluorene-3-carbonyl)glycyl)-4-(methylsulfonyl)pyrrolidine-2-carboxamide (Compound 400)

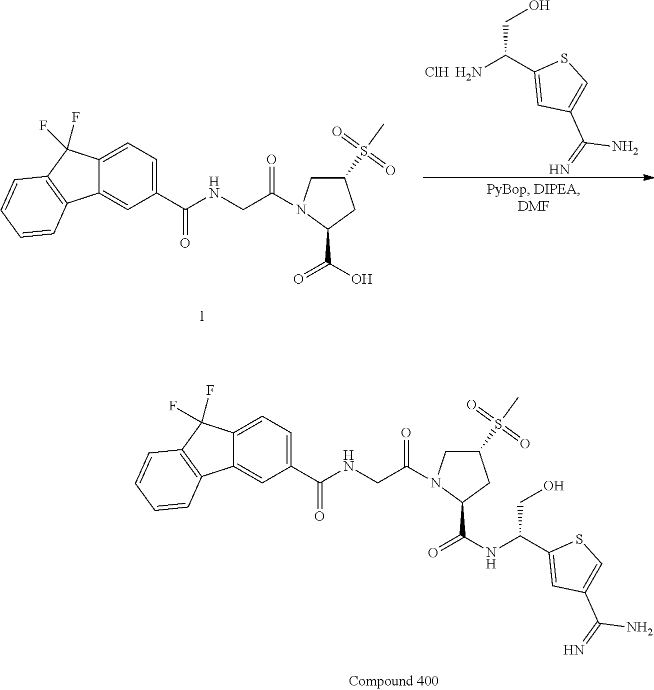 [SMILES: CS(=O)(=O)[C@@H]1C[C@@H](C(=O)O)N(C(=O)CNC(=O)c2ccc3c(c2)-c2ccccc2C3(F)F)C1 ; Cl ; N=C(N)c1csc([C@H](N)CO)c1 ; CS(=O)(=O)[C@@H]1C[C@@H](C(=O)N[C@H](CO)c2cc(C(=N)N)cs2)N(C(=O)CNC(=O)c2ccc3c(c2)-c2ccccc2C3(F)F)C1]

Compound 400 was prepared from (2S,4R)-1-((9,9-difluoro-9H-fluorene-3-carbonyl)glycyl)-4-(methylsulfonyl)pyrrolidine-2-carboxylic acid and (R)-5-(1-amino-2-hydroxyethyl)thiophene-3-carboximidamide hydrochloride based on the procedures set forth in Scheme 3. ¹H NMR (400 MHz, CD₃OD) δ 8.54 (s, 1H), 8.23 (dd, J=23.9, 1.5 Hz, 1H), 8.15 (s, 1H), 7.88-7.82 (m, 1H), 7.78-7.67 (m, 2H), 7.64 (d, J=7.4 Hz, 1H), 7.55 (dd, J=13.7, 6.3 Hz, 2H), 7.43 (dd, J=9.6, 4.9 Hz, 1H), 5.29 (dt, J=39.4, 5.7 Hz, 1H), 4.80 (dd, J=8.6, 4.8 Hz, 1H), 4.29 (dt, J=29.3, 14.6 Hz, 2H), 4.21-4.06 (m, 3H), 3.94-3.80 (m, 2H), 3.07 (d, J=19.9 Hz, 3H), 2.92-2.74 (m, 1H), 2.64-2.46 (m, 1H). LC/MS (ESI) m/z: 646 (M+H)⁺.

### Scheme 375: Synthesis of (2S,4R)—N—((R)-1-(4-carbamimidoylthiophen-2-yl)-2-hydroxyethyl)-1-((9,9-dimethyl-9H-fluorene-3-carbonyl)glycyl)-4-fluoro-4-(fluoromethyl)pyrrolidine-2-carboxamide (Compound 401)

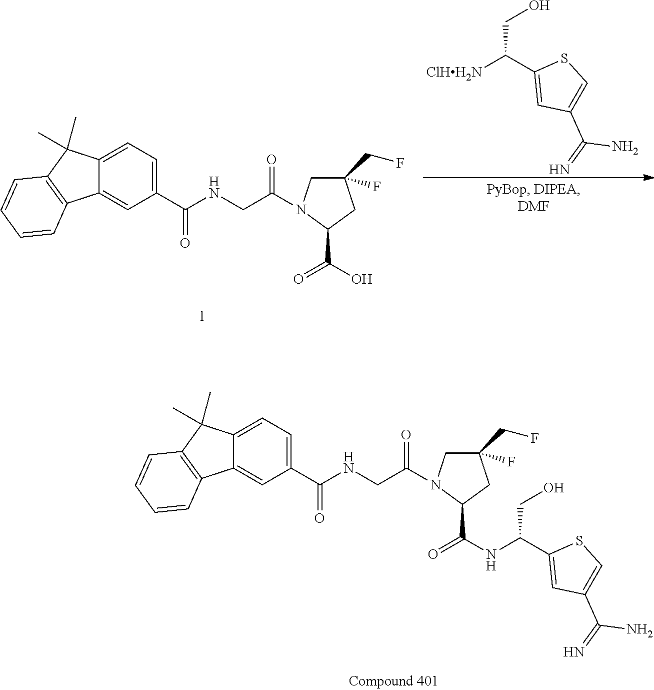

Compound 401 was prepared from (2S,4R)-1-((9,9-dimethyl-9H-fluorene-3-carbonyl)glycyl)-4-fluoro-4-(fluoromethyl)pyrrolidine-2-carboxylic acid and (R)-5-(1-amino-2-hydroxyethyl)thiophene-3-carboximidamide hydrochloride based on the procedures set forth in Scheme 3. ¹H NMR (400 MHz, CD₃OD) δ 8.52 (s, 1H), 8.28-8.22 (m, 2H), 7.81 (ddd, J=12.3, 6.8, 2.1 Hz, 2H), 7.58 (d, J=8.2 Hz, 2H), 7.51 (dd, J=5.9, 2.7 Hz, 1H), 7.36 (dd, J=5.2, 3.5 Hz, 2H), 5.24 (t, J=5.7 Hz, 1H), 4.77 (dd, J=19.8, 11.7 Hz, 2H), 4.66 (dd, J=13.4, 7.3 Hz, 1H), 4.36 (d, J=16.7 Hz, 1H), 4.21-4.11 (m, 2H), 3.91 (ddd, J=19.0, 9.5, 8.0 Hz, 3H), 2.63 (dd, J=24.4, 16.3 Hz, 1H), 2.26 (ddd, J=34.7, 14.3, 9.2 Hz, 1H), 1.50 (s, 6H). LC/MS (ESI) m/z: 610 (M+H)⁺.

### Scheme 376: Synthesis of (2S,4R)—N—((R)-1-(4-carbamimidoylthiophen-2-yl)-2-hydroxyethyl)-1-((4′-fluoro-[1,1′-biphenyl]-3-carbonyl)glycyl)-4-(methylsulfonyl)pyrrolidine-2-carboxamide (Compound 402)

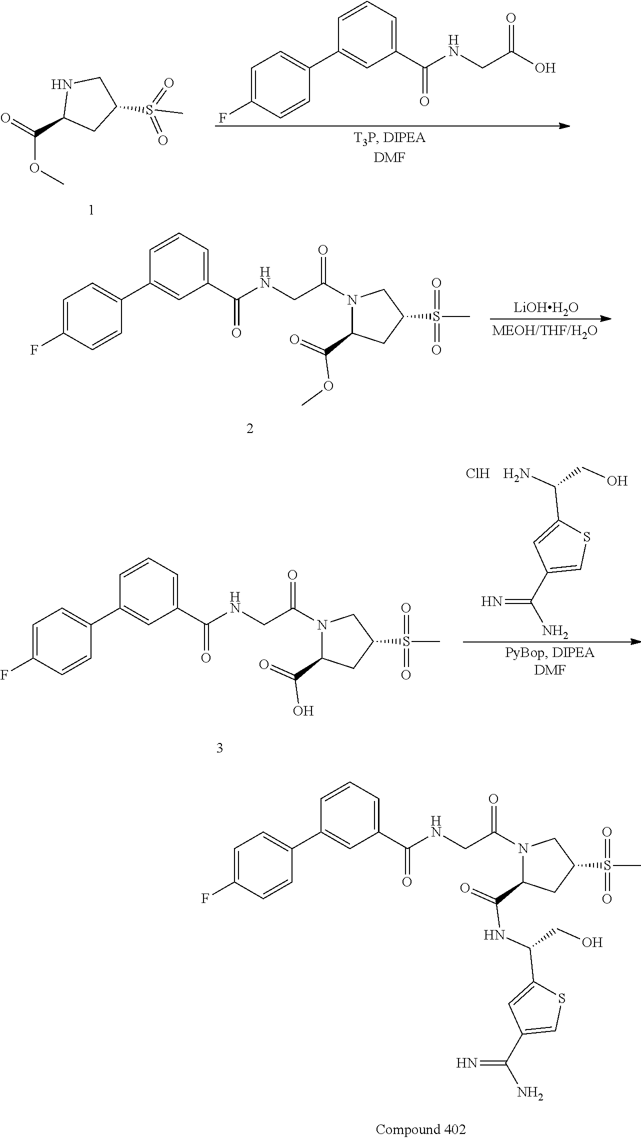

Compound 402 was prepared from methyl (2S,4R)-4-(methylsulfonyl)pyrrolidine-2-carboxylate, (4′-fluoro-[1,1′-biphenyl]-3-carbonyl)glycine, and (R)-5-(1-amino-2-hydroxyethyl)thiophene-3-carboximidamide hydrochloride based on procedures set forth in Scheme 3. ¹H NMR (400 MHz, CD₃OD) δ 8.54 (s, 1H), 8.27-8.20 (m, 1H), 8.09 (dd, J=8.0, 1.9 Hz, 1H), 7.84-7.78 (m, 2H), 7.72-7.67 (m, 2H), 7.58-7.53 (m, 2H), 7.23-7.17 (m, 2H), 5.36-5.19 (m, 1H), 4.79 (dd, J=8.6, 4.7 Hz, 1H), 4.30 (d, J=1.7 Hz, 1H), 4.22-4.11 (m, 3H), 4.09-3.78 (m, 3H), 3.09-3.04 (m, 3H), 2.80-2.45 (m, 2H). LC/MS (ESI) m/z: 616 (M+H)⁺.

### Scheme 377: Synthesis of (2S,4R)—N—((R)-1-(4-carbamimidoylthiophen-2-yl)-2-hydroxyethyl)-1-((9,9-difluoro-9H-fluorene-3-carbonyl)glycyl)-4-(difluoromethoxy) pyrrolidine-2-carboxamide (Compound 403)

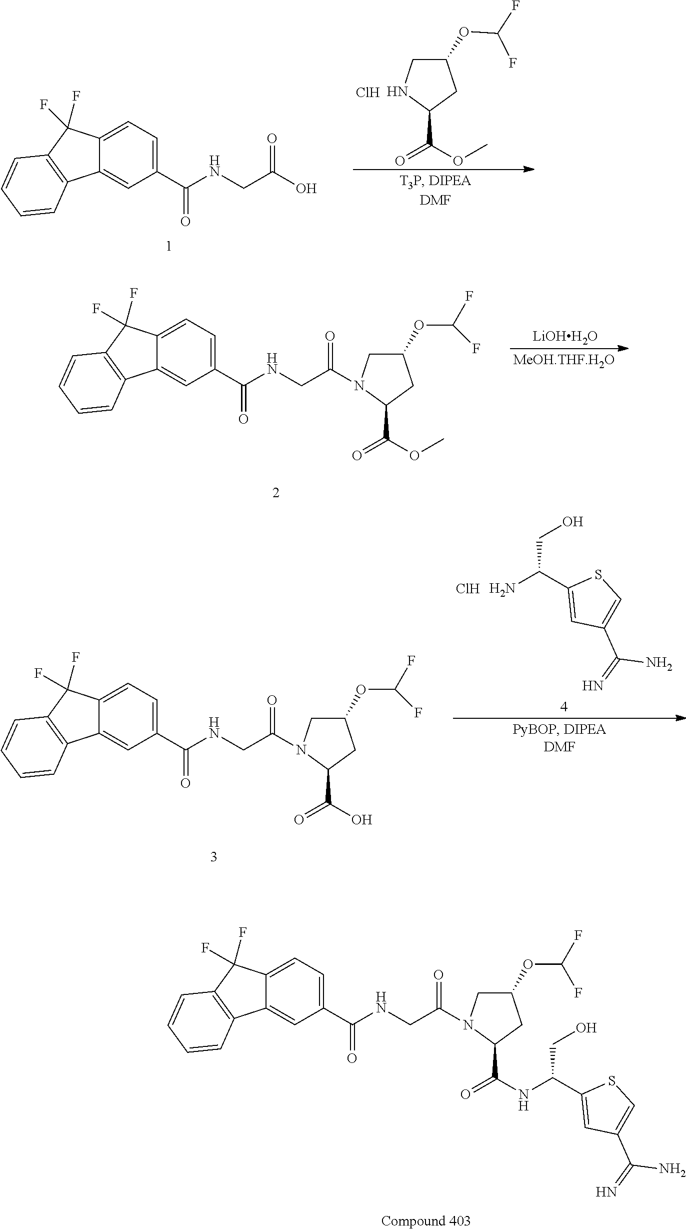

Compound 406 was prepared from (9,9-difluoro-9H-fluorene-3-carbonyl)glycine, methyl (2S,4R)-4-(difluoromethoxy)pyrrolidine-2-carboxylate hydrochloride, and (R)-5-(1-amino-2-hydroxyethyl)thiophene-3-carboximidamide hydrochloride based on the procedures set forth in Scheme 3. ¹H NMR (400 MHz, CD₃OD) δ 8.49 (s, 1H), 8.24 (t, J=7.7 Hz, 1H), 8.17 (d, J=8.3 Hz, 1H), 7.88 (d, J=7.9 Hz, 1H), 7.79-7.74 (m, 1H), 7.72 (d, J=7.8 Hz, 1H), 7.65 (d, J=7.4 Hz, 1H), 7.58 (d, J=4.1 Hz, 2H), 7.46 (d, J=7.5 Hz, 1H), 6.72-6.33 (m, 1H), 5.29 (dt, J=44.5, 5.9 Hz, 1H), 5.01 (d, 1H), 4.77-4.65 (m, 1H), 4.33 (d, J=16.8 Hz, 1H), 4.22-4.15 (m, 1H), 4.00-3.95 (m, 1H), 3.91 (s, 1H), 3.87-3.76 (m, 2H), 2.51 (dd, J=11.4, 7.4 Hz, 1H), 2.33-2.23 (m, 1H). LC/MS (ESI) m/z: 634 (M+H)⁺.

### Scheme 378: Synthesis of (2S,4R)—N—((R)-1-(4-carbamimidoylthiophen-2-yl)-2-hydroxyethyl)-1-((4′-methyl-[1,1′-biphenyl]-3-carbonyl)glycyl)-4-(methylsulfonyl) pyrrolidine-2-carboxamide (Compound 404)

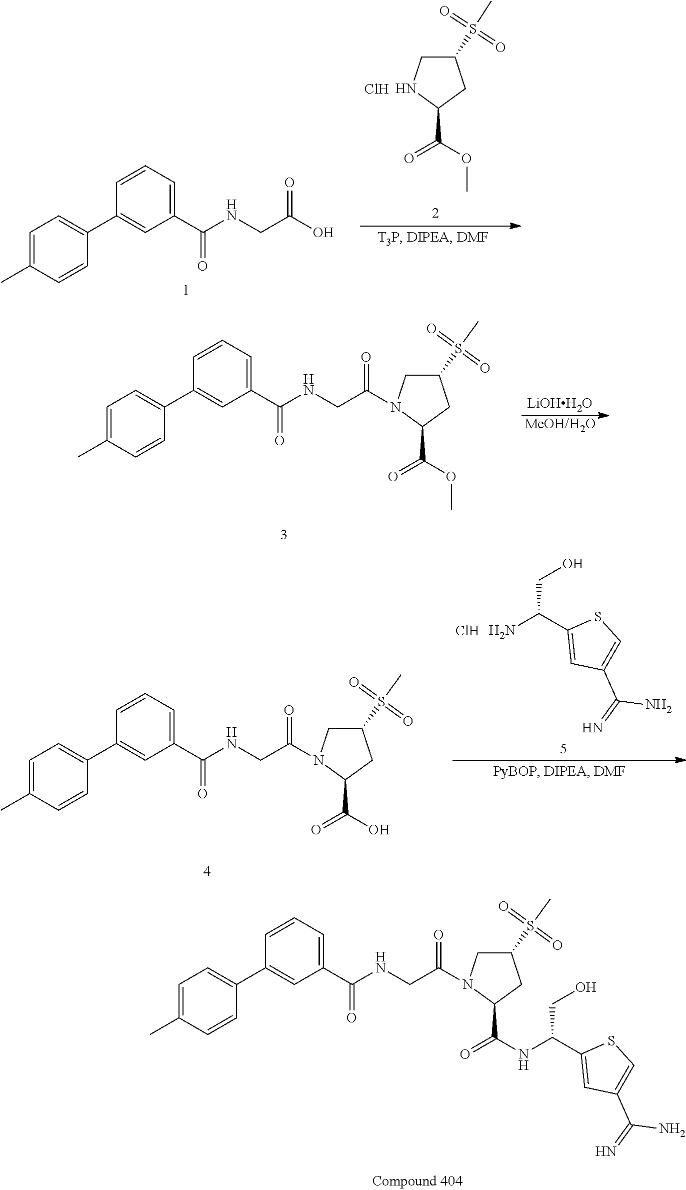

Compound 404 was prepared from (4′-methyl-[1,1′-biphenyl]-3-carbonyl)glycine, methyl (2S,4R)-4-(methylsulfonyl)pyrrolidine-2-carboxylate hydrochloride, and (R)-5-(1-amino-2-hydroxyethyl)thiophene-3-carboximidamide hydrochloride based on the procedures set forth in Scheme 3. ¹H NMR (400 MHz, CD₃OD) δ 8.54 (s, 1H), 8.27-8.16 (m, 1H), 8.09 (d, J=6.7 Hz, 1H), 7.79 (dd, J=9.8, 3.9 Hz, 2H), 7.57-7.51 (m, 4H), 7.28 (d, J=7.8 Hz, 2H), 5.28 (dt, J=40.6, 5.9 Hz, 1H), 4.79 (dd, J=8.7, 4.7 Hz, 1H), 4.25 (d, J=31.6 Hz, 2H), 4.20-4.08 (m, 3H), 3.94-3.81 (m, 2H), 3.06 (d, J=18.9 Hz, 3H), 2.83 (ddd, J=19.9, 12.3, 5.2 Hz, 1H), 2.61-2.45 (m, 1H), 2.38 (s, 3H). LC/MS (ESI) m/z: 612 (M+H)⁺.

### Scheme 379: (2S,4R)—N—((R)-1-(4-carbamimidoylthiophen-2-yl)-2-hydroxyethyl)-1-((9,9-difluoro-9H-fluorene-2-carbonyl)glycyl)-4-fluoro-4-(fluoromethyl)pyrrolidine-2-carboxamide (Compound 405)

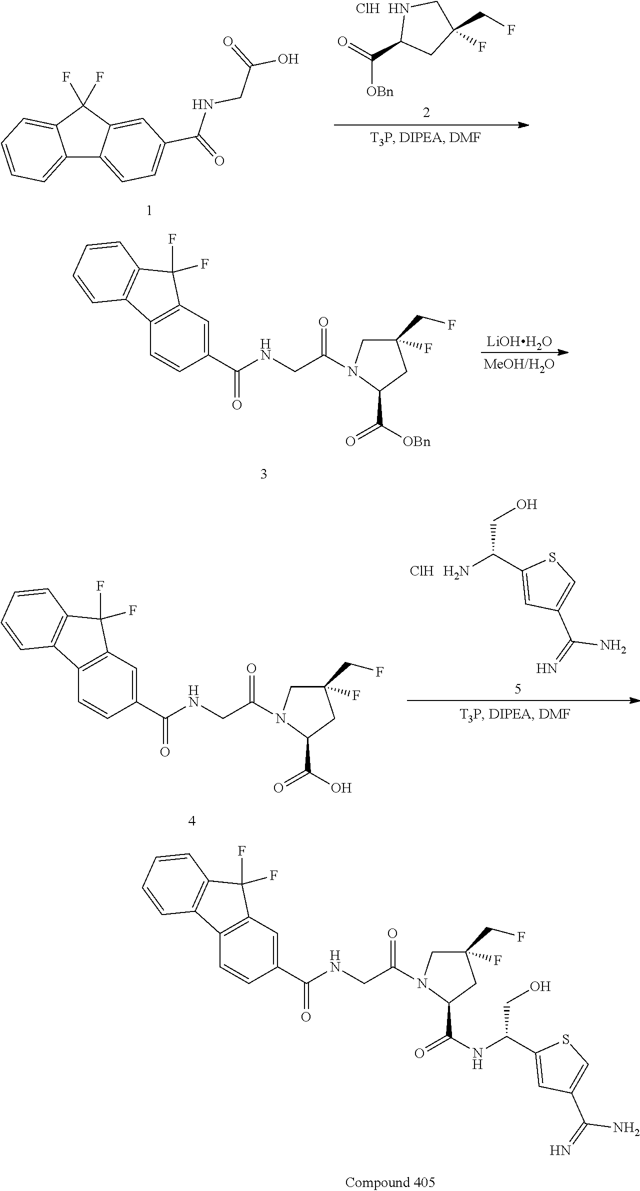

Compound 405 was prepared from (9,9-difluoro-9H-fluorene-2-carbonyl)glycine, benzyl (2S,4R)-4-fluoro-4-(fluoromethyl)pyrrolidine-2-carboxylate hydrochloride, and (R)-5-(1-amino-2-hydroxyethyl)thiophene-3-carboximidamide hydrochloride based on the procedures set forth in Scheme 66. ¹H NMR (400 MHz, CD₃OD) δ 8.54 (s, 1H), 8.25 (t, J=9.2 Hz, 1H), 8.11 (d, J=6.4 Hz, 1H), 8.05 (d, J=7.8 Hz, 1H), 7.81 (t, J=8.0 Hz, 2H), 7.67 (d, J=7.5 Hz, 1H), 7.61-7.55 (m, 2H), 7.47 (t, J=7.4 Hz, 1H), 5.38-5.21 (m, 1H), 4.82-4.74 (m, 2H), 4.73-4.60 (m, 2H), 4.35 (d, J=16.7 Hz, 1H), 4.14 (dd, J=17.4, 10.8 Hz, 2H), 3.95-3.84 (m, 2H), 2.74-2.54 (m, 1H), 2.26 (ddd, J=34.7, 14.2, 9.1 Hz, 1H). LCMS (ESI) m/z: 618 (M+H)⁺.

### Scheme 380: Synthesis of (2S,4R)—N—((R)-1-(4-carbamimidoylthiophen-2-yl)-2-hydroxyethyl)-1-((9,9-difluoro-9H-fluorene-2-carbonyl)glycyl)-4-(methylsulfonyl)pyrrolidine-2-carboxamide (Compound 406)

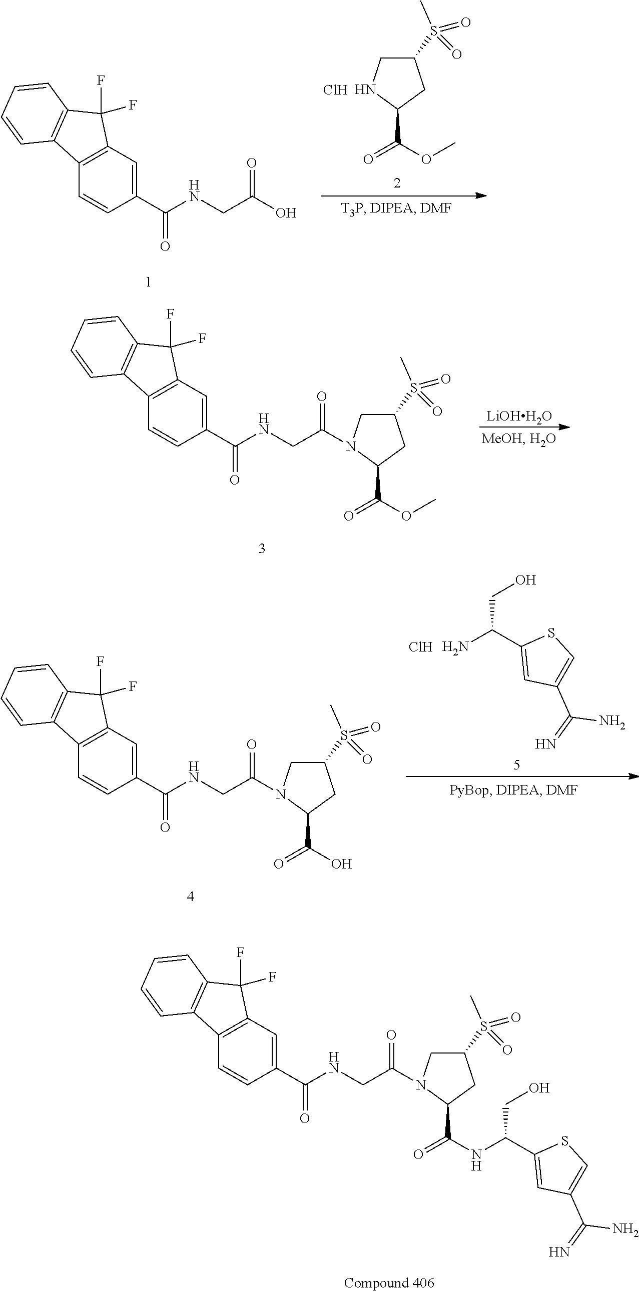

Compound 406 was prepared from (9,9-difluoro-9H-fluorene-2-carbonyl)glycine, methyl (2S,4R)-4-(methylsulfonyl)pyrrolidine-2-carboxylate hydrochloride, and (R)-5-(1-amino-2-hydroxyethyl)thiophene-3-carboximidamide hydrochloride based on the procedures set forth in Scheme 3. ¹H NMR (400 MHz, CD₃OD) δ 8.55 (s, 1H), 8.25 (dd, J=17.8, 1.4 Hz, 1H), 8.11 (d, J=6.2 Hz, 1H), 8.04 (t, J=6.5 Hz, 1H), 7.80 (dd, J=13.1, 5.5 Hz, 2H), 7.66 (d, J=7.4 Hz, 1H), 7.57 (dd, J=14.6, 7.0 Hz, 2H), 7.47 (t, J=7.5 Hz, 1H), 5.37-5.22 (m, 1H), 4.80 (dd, J=8.6, 4.7 Hz, 1H), 4.34-4.15 (m, 4H), 4.13-3.96 (m, 1H), 3.95-3.79 (m, 2H), 3.08 (d, J=19.8 Hz, 3H), 2.94-2.73 (m, 1H), 2.66-2.46 (m, 1H). LCMS (ESI) m/z: 646 (M+H)⁺.

### Scheme 381: Synthesis of (2S,4R)—N—((R)-1-(4-carbamimidoylthiophen-2-yl)-2-hydroxyethyl)-1-((9,9-difluoro-9H-fluorene-3-carbonyl)glycyl)-4-methoxypyrrolidine-2-carboxamide (Compound 407)

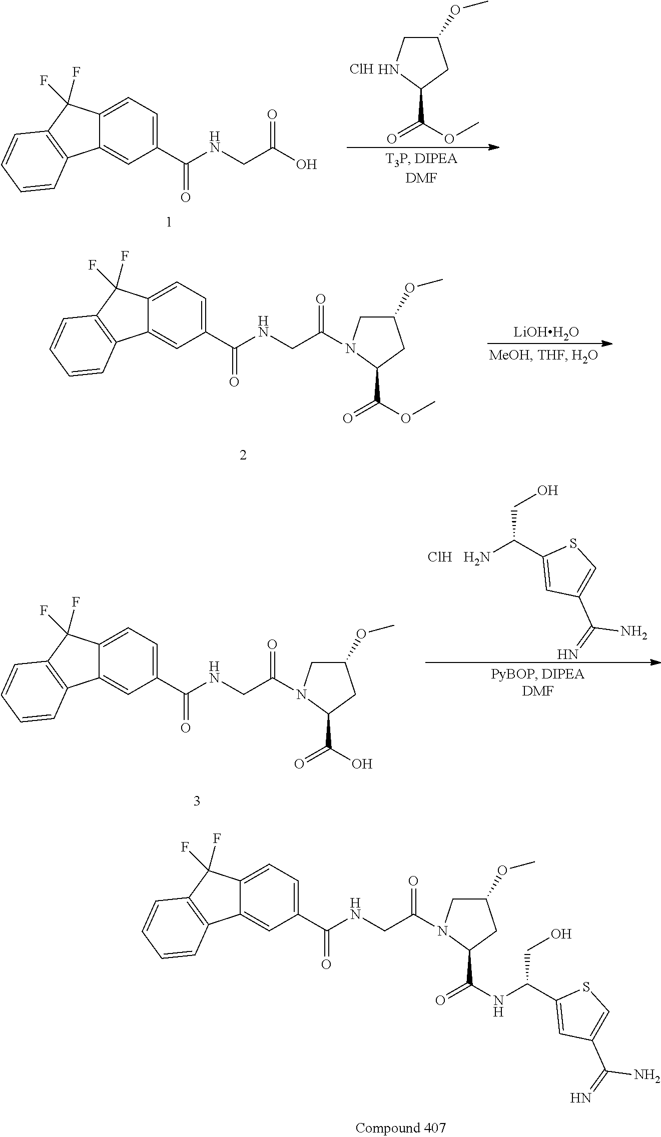

Compound 407 was prepared from (9,9-difluoro-9H-fluorene-3-carbonyl)glycine, methyl (2S,4R)-4-methoxypyrrolidine-2-carboxylate hydrochloride, and (R)-5-(1-amino-2-hydroxyethyl)thiophene-3-carboximidamide hydrochloride based on the procedures set forth in Scheme 3. ¹H NMR (400 MHz, CD₃OD) δ 8.53 (s, 1H), 8.22 (dd, J=17.6, 16.2 Hz, 2H), 7.86 (t, J=7.6 Hz, 1H), 7.78-7.70 (m, 2H), 7.65 (d, J=7.6 Hz, 1H), 7.60-7.55 (m, 2H), 7.45 (t, J=7.4 Hz, 1H), 5.28 (dt, J=11.2, 6.1 Hz, 1H), 4.57 (t, J=8.1 Hz, 1H), 4.34 (d, J=16.7 Hz, 1H), 4.23-4.15 (m, 2H), 3.93-3.80 (m, 4H), 3.37 (d, J=20.4 Hz, 3H), 2.49-2.40 (m, 1H), 2.11 (ddd, J=13.3, 10.2, 6.0 Hz, 1H). LC/MS (ESI) m/z: 598 (M+H)⁺.

### Scheme 382: Synthesis of (2S,4R)—N—((R)-1-(4-carbamimidoylthiophen-2-yl)-2-hydroxyethyl)-1-((9,9-difluoro-9H-fluorene-3-carbonyl)glycyl)-4-fluoro-4-(methoxymethyl)pyrrolidine-2-carboxamide (Compound 408)

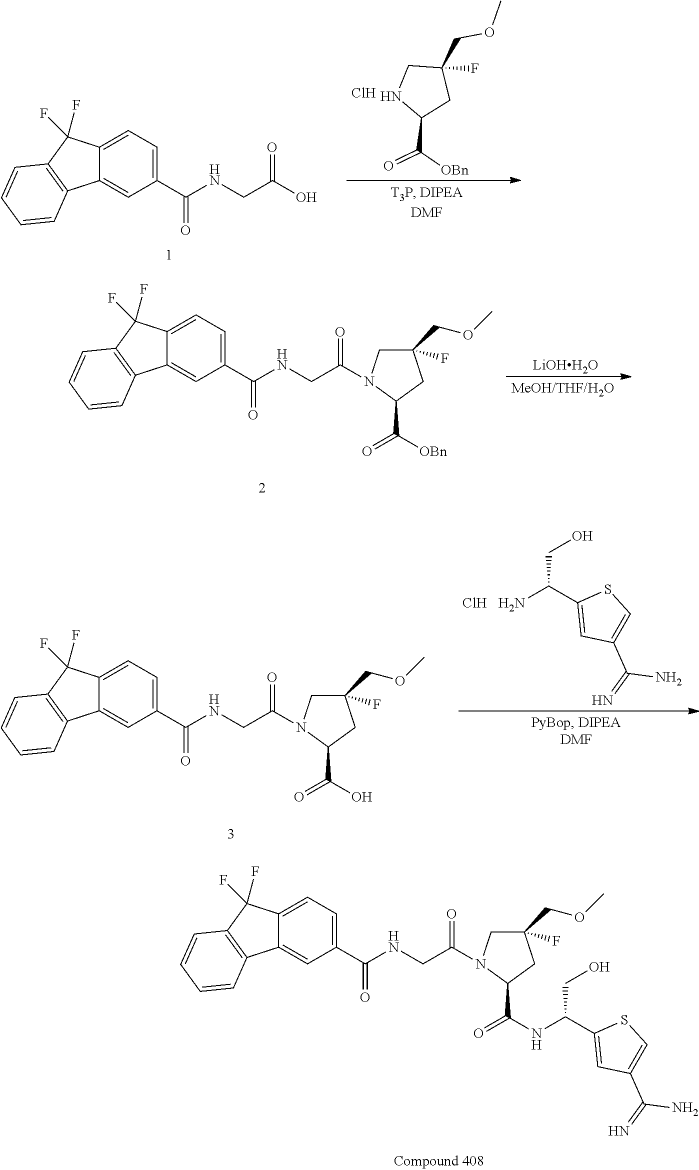

Compound 408 was prepared from (9,9-difluoro-9H-fluorene-3-carbonyl)glycine, benzyl (2S,4R)-4-fluoro-4-(methoxymethyl)pyrrolidine-2-carboxylate, and (R)-5-(1-amino-2-hydroxyethyl)thiophene-3-carboximidamide hydrochloride based on the procedures set forth in Scheme 3. ¹H NMR (400 MHz, CD₃OD) δ 8.50 (s, 1H), 8.27-8.17 (m, 2H), 7.90-7.86 (m, 1H), 7.74 (dd, J=16.5, 7.8 Hz, 2H), 7.66 (d, J=7.2 Hz, 1H), 7.58 (dd, J=12.0, 2.7 Hz, 2H), 7.47-7.43 (m, 1H), 5.27-5.20 (m, 1H), 4.70 (dd, J=9.3, 8.2 Hz, 1H), 4.36 (d, J=16.8 Hz, 1H), 4.16 (d, J=16.7 Hz, 1H), 4.12-4.02 (m, 1H), 3.98-3.90 (m, 1H), 3.87-3.81 (m, 2H), 3.74-3.66 (m, 2H), 3.44 (s, 3H), 2.65-2.51 (m, 1H), 2.31-2.14 (m, 1H). LC/MS (ESI) m/z: 630 (M+H)⁺.

### Scheme 383: Synthesis of (2S,4R)—N—((R)-1-(4-carbamimidoylthiophen-2-yl)-2-hydroxyethyl)-1-((9,9-dimethyl-9H-fluorene-3-carbonyl)glycyl)-4-fluoro-4-(methoxymethyl)pyrrolidine-2-carboxamide (Compound 409)

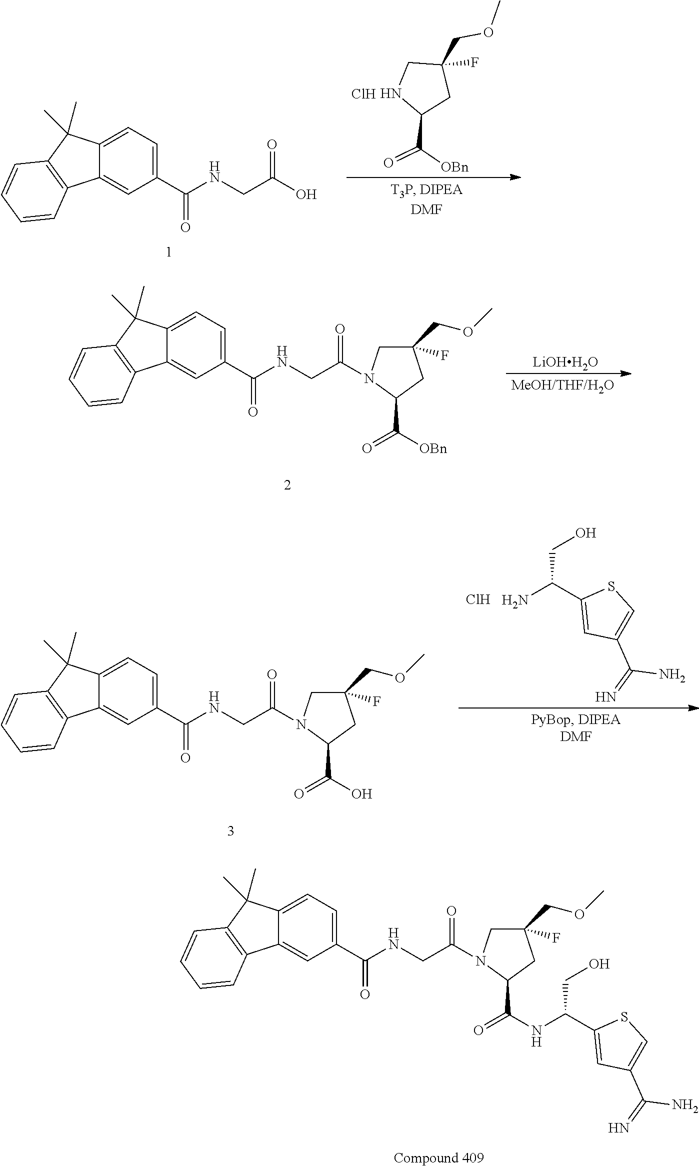

Compound 409 was prepared from (9,9-dimethyl-9H-fluorene-3-carbonyl)glycine, benzyl (2S,4R)-4-fluoro-4-(methoxymethyl)pyrrolidine-2-carboxylate hydrochloride, and (R)-5-(1-amino-2-hydroxyethyl)thiophene-3-carboximidamide hydrochloride based on the procedures set forth in Scheme 3. ¹H NMR (400 MHz, CD₃OD) δ 8.52 (s, 1H), 8.27-8.22 (m, 2H), 7.84-7.79 (m, 2H), 7.61-7.57 (m, 2H), 7.52-7.49 (m, 1H), 7.36 (dd, J=5.2, 3.4 Hz, 2H), 5.26 (dd, J=26.5, 20.5 Hz, 1H), 4.74-4.67 (m, 1H), 4.34 (d, J=16.7 Hz, 1H), 4.16 (d, J=16.5 Hz, 1H), 4.12-4.04 (m, 1H), 3.98-3.83 (m, 3H), 3.70 (dt, J=13.7, 5.6 Hz, 2H), 3.43 (d, J=5.9 Hz, 3H), 2.65-2.51 (m, 1H), 2.31-2.15 (m, 1H), 1.49 (s, 6H). LC/MS (ESI) m/z: 622 (M+H)⁺.

### Scheme 384: Synthesis of (1S,5R)—N—((R)-1-(4-carbamimidoylthiophen-2-yl)ethyl)-2-((9,9-difluoro-9H-fluorene-3-carbonyl)glycyl)-5-(2,2,2-trifluoroethyl)-3λ³-2-azabicyclo[3.1.0]hexane-3-carboxamide (Compound 410)

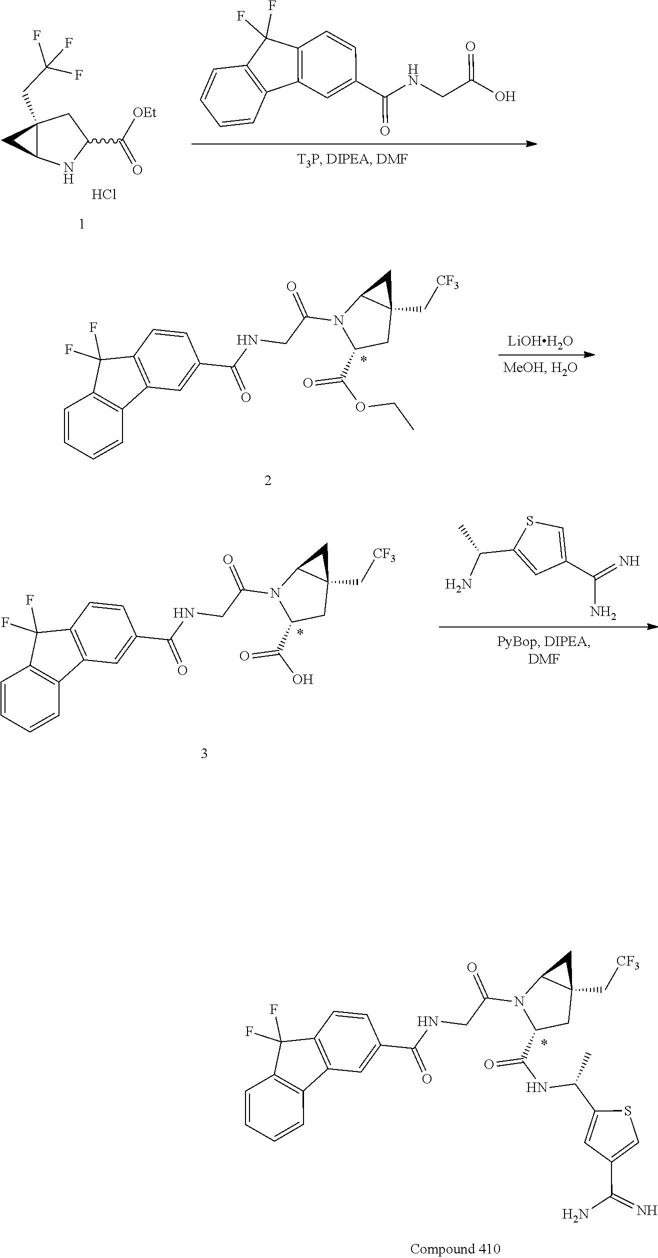

Compound 410 was prepared from (9,9-difluoro-9H-fluorene-3-carbonyl)glycine, ethyl (1S,5S)-5-(2,2,2-trifluoroethyl)-2-azabicyclo[3.1.0]hexane-3-carboxylate hydrochloride, and (R)-5-(1-aminoethyl)thiophene-3-carboximidamide based on the procedures set forth in Scheme 3. ¹H NMR (400 MHz, CD₃OD) δ 8.51 (s, 1H), 8.34-8.14 (m, 2H), 7.97-7.63 (m, 5H), 7.60-7.55 (m, 1H), 7.47-7.43 (m, 1H), 5.79-5.63 (m, 1H), 5.32 (dd, J=20.0, 15.3 Hz, 1H), 4.55 (dd, J=14.6, 6.6 Hz, 2H), 4.45-4.36 (m, 1H), 4.03 (s, 1H), 3.74-3.44 (m, 1H), 2.30-2.16 (m, 1H), 2.03 (d, J=5.1 Hz, 1H), 1.69-1.57 (m, 3H), 1.54 (d, J=6.7 Hz, 1H), 0.90 (t, J=6.9 Hz, 1H). LCMS (ESI) (m/z): 646 (M+H)⁺.

### Scheme 385: Synthesis of (1S,5R)—N—((R)-1-(4-carbamimidoylthiophen-2-yl)ethyl)-2-((9,9-difluoro-9H-fluorene-3-carbonyl)glycyl)-5-(2,2,2-trifluoroethyl)-3λ³-2-azabicyclo[3.1.0]hexane-3-carboxamide (Compound 411)

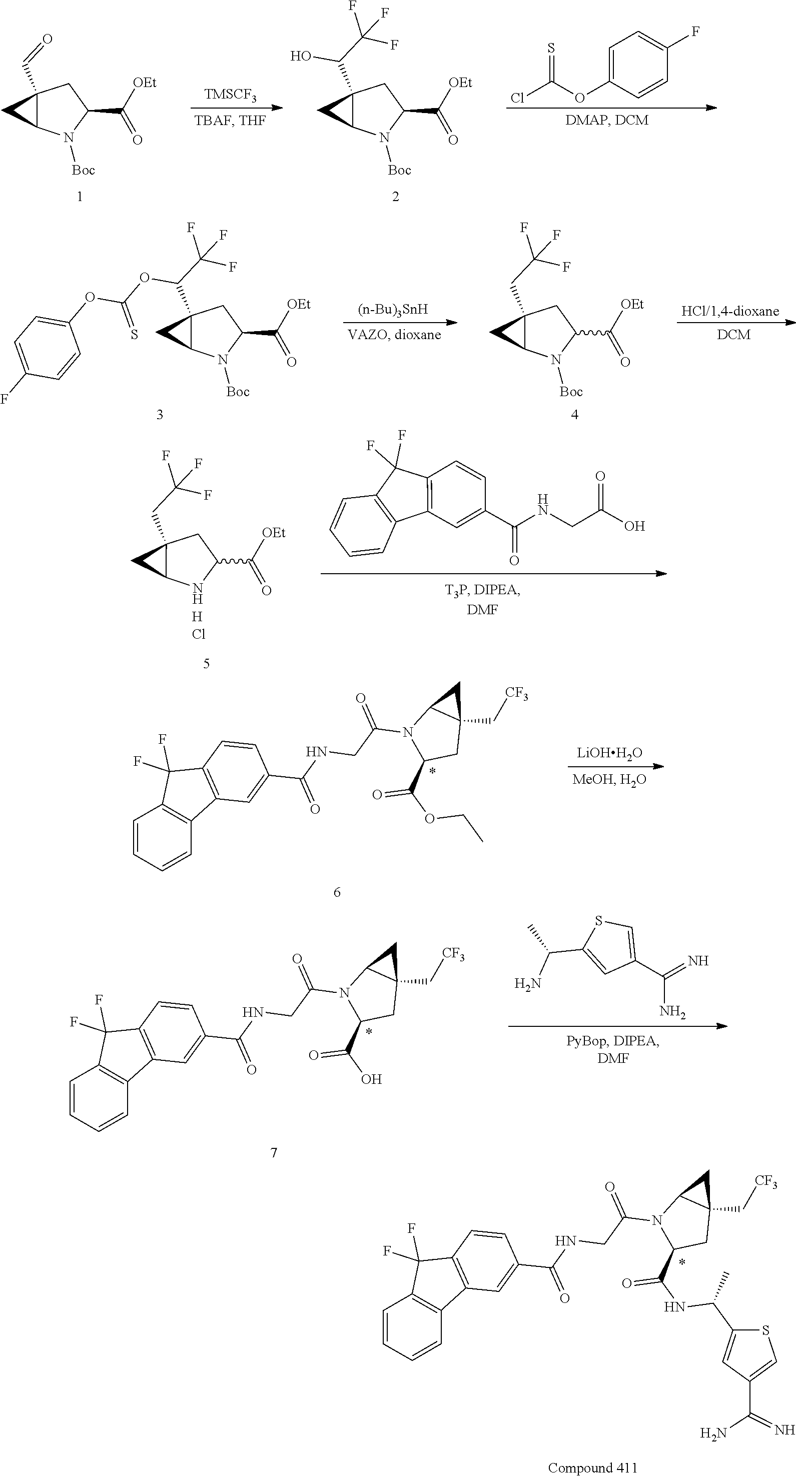

### Step 1: 2-(tert-butyl) 3-ethyl (1S,3S,5R)-5-((R)-2,2,2-trifluoro-1-hydroxyethyl)-2-azabicyclo[3.1.0]hexane-2,3-dicarboxylate

To a mixture of 2-(tert-butyl) 3-ethyl (1S,3S,5R)-5-formyl-2-azabicyclo[3.1.0]hexane-2,3-dicarboxylate (283 mg, 1.00 mmol) and TBAF (0.5 mL, 0.05 mmol, 1 N in THF) in THF (3 mL) was added TMSCF₃ (284 mg, 2.0 mmol) under N₂ atmosphere at 0° C. and the mixture was stirred at room temperature for 16 hours. The mixture was diluted with EtOAc, washed with saturated aq·NH₄Cl solution twice and brine, dried over anhydrous Na₂SO₄, filtered, and concentrated under reduced pressure to dryness. The residue was purified by flash chromatography (silica gel, 0-25% EtOAc in PE) to give 2-(tert-butyl) 3-ethyl (1 S,3S,5R)-5-((R)-2,2,2-trifluoro-1-hydroxyethyl)-2-azabicyclo[3.1.0]hexane-2,3-dicarboxylate (205 mg, yield 58.1%) as a colorless oil. LCMS (ESI) (m/z): 354 (M+H)⁺.

### Step 2: 2-(tert-butyl) 3-ethyl (1S,3S,5R)-5-((R)-2,2,2-trifluoro-1-(((4-fluorophenoxy) carbonothioyl)oxy)ethyl)-2-azabicyclo[3.1.0]hexane-2,3-dicarboxylate

To a mixture of 2-(tert-butyl) 3-ethyl (1S,3S,5R)-5-((R)-2,2,2-trifluoro-1-hydroxyethyl)-2-azabicyclo[3.1.0]hexane-2,3-dicarboxylate (200 mg, 0.57 mmol) and O-(4-fluorophenyl) carbonochloridothioate (108 mg, 0.57 mmol) in DCM (5 mL) was added DMAP (140 mg, 1.14 mmol) and the mixture was stirred at 25° C. for 5 hours. The mixture was diluted with EtOAc, washed with 0.5N aq·HCl solution, dried over anhydrous Na₂SO₄, filtered, and concentrated under reduced pressure to dryness. The residue was purified by flash chromatography (silica gel, 0-15% EtOAc in PE) to give 2-(tert-butyl) 3-ethyl (1 S,3S,5R)-5-((R)-2,2,2-trifluoro-1-(((4-fluorophenoxy)carbonothioyl)oxy)ethyl)-2-azabicyclo[3.1.0]hexane-2,3-dicarboxylate (189 mg, yield 65.8%) as a colorless oil. LCMS (ESI) m/z: 508 (M+H)⁺.

### Step 3: 2-(tert-butyl) 3-ethyl (1S,5S)-5-(2,2,2-trifluoroethyl)-2-azabicyclo[3.1.0]hexane-2,3-dicarboxylate

To a mixture of 2-(tert-butyl) 3-ethyl (1S,3S,5R)-5-((R)-2,2,2-trifluoro-1-(((4-fluorophenoxy)carbonothioyl)oxy)ethyl)-2-azabicyclo[3.1.0]hexane-2,3-dicarboxylate (190 mg, 0.37 mmol) and (n-Bu)₃SnH (273 mg, 0.93 mmol) in 1,4-dioxane (4 mL) was added VAZO (31 mg, 0.18 mmol) and the reaction mixture was stirred at 105° C. under N₂ atmosphere for 0.5 hour. The mixture was quenched with saturated aq·KF solution and extracted with EtOAc twice. The combined organic layers were washed with brine, dried over anhydrous Na₂SO₄, filtered, and concentrated under reduced pressure to dryness. The residue was purified by flash chromatography (silica gel, 0-20% EtOAc in PE) to give 2-(tert-butyl) 3-ethyl (1S,5S)-5-(2,2,2-trifluoroethyl)-2-azabicyclo[3.1.0]hexane-2,3-dicarboxylate (98 mg, yield 77.8%) as a colorless oil. LCMS (ESI) m/z: 338 (M+H)⁺.

### Step 4: ethyl (1S,5S)-5-(2,2,2-trifluoroethyl)-2-azabicyclo[3.1.0]hexane-3-carboxylate hydrochloride

To a solution of 2-(tert-butyl) 3-ethyl (1S,5S)-5-(2,2,2-trifluoroethyl)-2-azabicyclo [3.1.0]hexane-2,3-dicarboxylate (98 mg, 0.29 mmol) in DCM (1 mL) was added HCl/1,4-dioxane (3 mL, 4M) and the mixture was stirred under N₂ atmosphere at room temperature for 2 hours. The reaction mixture was concentrated to give ethyl (1 S,5S)-5-(2,2,2-trifluoroethyl)-2-azabicyclo[3.1.0]hexane-3-carboxylate hydrochloride (60 mg, yield 89.5%) as a brown oil. LCMS (ESI) (m/z): 238 (M+H)⁺.

### Step 5: ethyl (1S,5R)-2-((9,9-difluoro-9H-fluorene-3-carbonyl)glycyl)-5-(2,2,2-trifluoroethyl)-3λ³-2-azabicyclo[3.1.0]hexane-3-carboxylate

To a mixture of (9,9-difluoro-9H-fluorene-3-carbonyl)glycine (86 mg, 0.28 mmol) and ethyl (1S,5S)-5-(2,2,2-trifluoroethyl)-2-azabicyclo[3.1.0]hexane-3-carboxylate hydrochloride (67 mg, 0.28 mmol) in DMF (3 mL) was added DIPEA (220 mg, 1.68 mmol) and T₃P (542 mg, 0.84 mmol, 50% wt. in EtOAc) under N₂ atmosphere at 0° C. and the mixture was stirred at room temperature for 2 hours. The mixture was quenched with saturated aq·NaHCO₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give ethyl (1S,5R)-2-((9,9-difluoro-9H-fluorene-3-carbonyl)glycyl)-5-(2,2,2-trifluoroethyl)-3λ³-2-azabicyclo[3.1.0]hexane-3-carboxylate (19 mg, yield 12.3%) as a colorless oil. LCMS (ESI) (m/z): 523 (M+H)⁺.

### Step 6: (1S,5R)-2-((9,9-difluoro-9H-fluorene-3-carbonyl)glycyl)-5-(2,2,2-trifluoroethyl)-3λ³-2-azabicyclo[3.1.0]hexane-3-carboxylic acid

To a solution of ethyl (1 S,5R)-2-((9,9-difluoro-9H-fluorene-3-carbonyl)glycyl)-5-(2,2,2-trifluoroethyl)-3λ³-2-azabicyclo[3.1.0]hexane-3-carboxylate (19 mg, 0.03 mmol) in MeOH (3 mL) and H₂O (1 mL) was added LiOH·H₂O (3 mg, 0.06 mmol) and the mixture was stirred at room temperature for 2 hours. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (1S,5R)-2-((9,9-difluoro-9H-fluorene-3-carbonyl)glycyl)-5-(2,2,2-trifluoroethyl)-3λ³-2-azabicyclo[3.1.0]hexane-3-carboxylic acid (15 mg, yield 88.2%) as a brown solid, which was used directly in the next step without further purification. LC/MS (ESI) m/z: 495 (M+H)⁺.

### Step 7: (1S,5R)—N—((R)-1-(4-carbamimidoylthiophen-2-yl)ethyl)-2-((9,9-difluoro-9H-fluorene-3-carbonyl)glycyl)-5-(2,2,2-trifluoroethyl)-3λ³-2-azabicyclo[3.1.0]hexane-3-carboxamide (Compound 411)

To a mixture of (1S,5R)-2-((9,9-difluoro-9H-fluorene-3-carbonyl)glycyl)-5-(2,2,2-trifluoroethyl)-3λ³-2-azabicyclo[3.1.0]hexane-3-carboxylic acid (15 mg, 0.03 mmol) and (R)-5-(1-aminoethyl)thiophene-3-carboximidamide (10 mg, 0.06 mmol) in DMF (3 mL) was added DIPEA (23 mg, 0.18 mmol) and PyBop (23 mg, 0.05 mmol) under N₂ atmosphere at 0° C. and the mixture was stirred at room temperature for 2 hours. The mixture was diluted with EtOAc, washed with saturated aq·NH₄Cl solution and brine, dried over anhydrous Na₂SO₄, filtered, and concentrated under reduced pressure to dryness. The residue was purified by prep-TLC (DCM:MeOH=8:1) and further purified by prep-HPLC to give Compound 411 (1.8 mg, yield 9.5%) as a white solid. ¹H NMR (400 MHz, CD₃OD) δ 8.53 (s, 1H), 8.27-8.07 (m, 2H), 7.88 (dd, J=18.1, 7.4 Hz, 1H), 7.78 (t, J=8.8 Hz, 1H), 7.75-7.60 (m, 3H), 7.58 (t, J=7.6 Hz, 1H), 7.45 (t, J=7.6 Hz, 1H), 5.88-5.52 (m, 1H), 5.35 (t, J=5.1 Hz, 1H), 4.57 (d, J=10.0 Hz, 2H), 4.47 (s, 1H), 4.03 (d, J=12.1 Hz, 1H), 3.70 (t, J=15.2 Hz, 1H), 2.27-2.12 (m, 1H), 2.03 (d, J=5.2 Hz, 1H), 1.65-1.58 (m, 3H), 1.32 (d, J=3.1 Hz, 1H), 0.90 (t, J=6.9 Hz, 1H). LCMS (ESI) (m/z): 646 (M+H)⁺.

### Scheme 386: Synthesis of (2S,4R)—N—((R)-1-(4-carbamimidoylthiophen-2-yl)ethyl)-1-((4-(4-fluorophenoxy)benzoyl)glycyl)-4-(thiazol-2-yl)pyrrolidine-2-carboxamide (Compound 412)

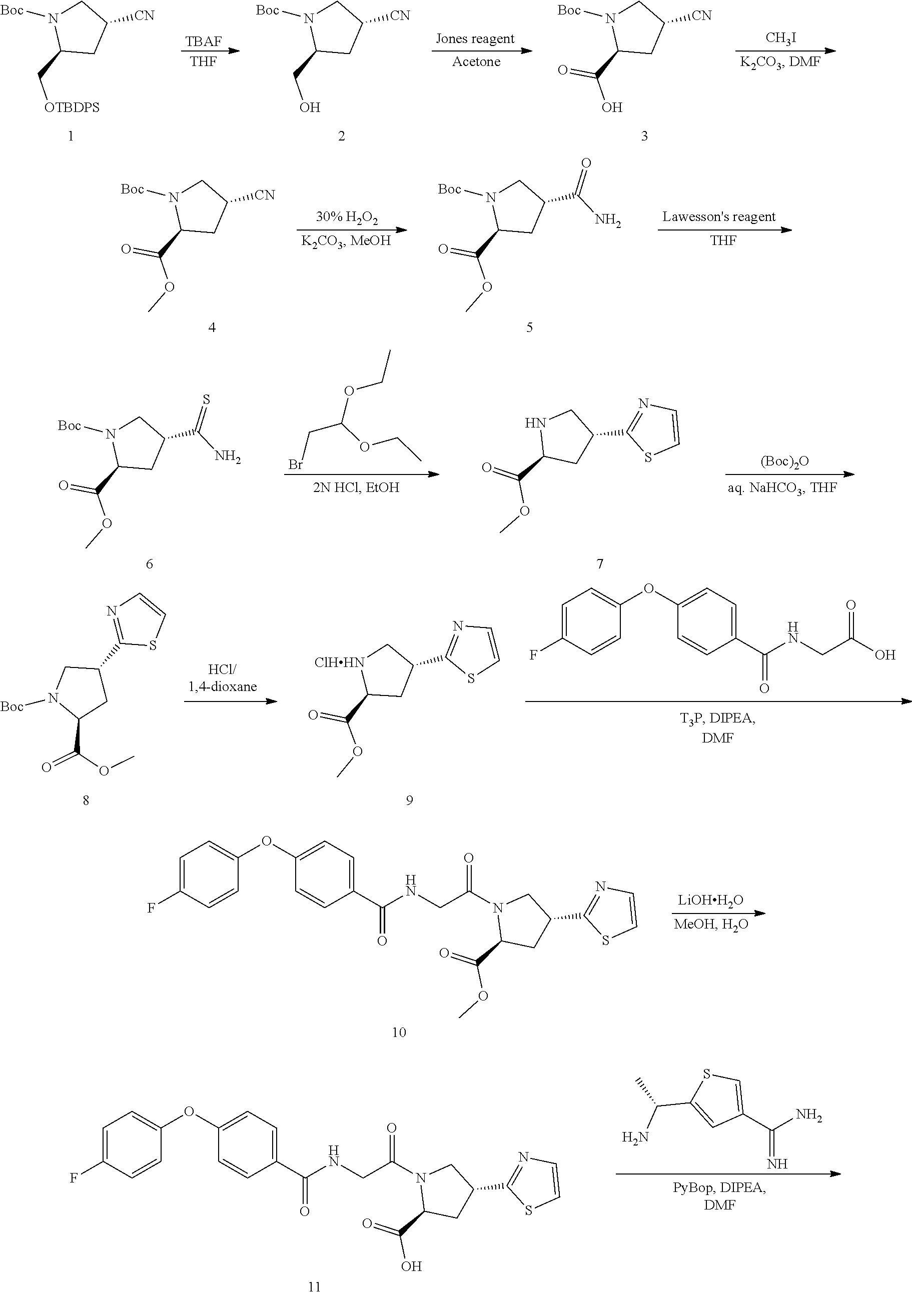

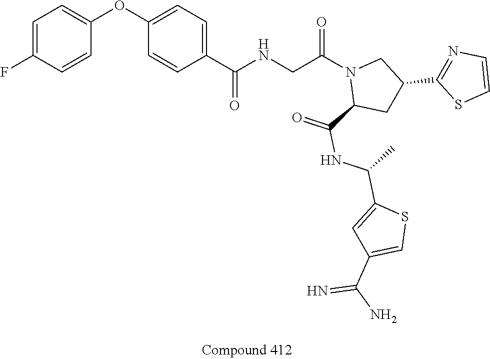 [SMILES: C[C@@H](NC(=O)[C@@H]1C[C@@H](c2nccs2)CN1C(=O)CNC(=O)c1ccc(Oc2ccc(F)cc2)cc1)c1cc(C(=N)N)cs1]

### Step 1: tert-butyl (2S,4R)-4-cyano-2-(hydroxymethyl)pyrrolidine-1-carboxylate

To a solution of tert-butyl (2S,4R)-2-(((tert-butyldiphenylsilyl)oxy)methyl)-4-cyanopyrrolidine-1-carboxylate (8.10 g, 17.46 mmol) in THE (50 mL) was added TBAF (26.2 mL, 26.2 mmol, 1 M in THF) drop-wisely at 0° C. under N₂ atmosphere and the reaction mixture was stirred at room temperature for 2 hours. The mixture was diluted with EtOAc, washed with saturated aq·NH₄Cl solution five times, dried over anhydrous Na₂SO₄, filtered, and concentrated under reduced pressure to dryness. The residue was purified by flash chromatography (silica gel, 0-45% EtOAc in PE) to give tert-butyl (2S,4R)-4-cyano-2-(hydroxymethyl)pyrrolidine-1-carboxylate (3.58 g, yield 90.8%) as a colorless oil. LC/MS (ESI) m/z: 227 (M+H)⁺.

### Step 2: (2S,4R)-1-(tert-butoxycarbonyl)-4-cyanopyrrolidine-2-carboxylic acid

To a solution of tert-butyl (2S,4R)-4-cyano-2-(hydroxymethyl)pyrrolidine-1-carboxylate (3.50 g, 15.49 mmol) in acetone (35 mL) was added Jones reagent (9 mL, newly prepared) at 0° C. and the reaction mixture was stirred at 25° C. for 2 hours. The mixture was poured into ice-water and extracted with EtOAc twice. The combined organic layers were washed with saturated aq·Na₂S₂O₃ solution and brine, dried over anhydrous Na₂SO₄, filtered, and concentrated to dryness to give (2S,4R)-1-(tert-butoxycarbonyl)-4-cyanopyrrolidine-2-carboxylic acid (3.10 g, yield 83.8%) as a colorless oil. LC/MS (ESI) m/z: 241 (M+H)⁺.

### Step 3: 1-(tert-butyl) 2-methyl (2S,4R)-4-cyanopyrrolidine-1,2-dicarboxylate

To a mixture of (2S,4R)-1-(tert-butoxycarbonyl)-4-cyanopyrrolidine-2-carboxylic acid (3.06 g, 12.75 mmol) and K₂CO₃ (5.28 g, 38.25 mmol) in DMF (30 mL) was added CH₃I (2.72 g, 19.13 mmol) at 0° C. and the mixture was stirred at room temperature for 16 hours. The mixture was diluted with EtOAc, washed with saturated aq·NH₄Cl solution and brine, dried over anhydrous Na₂SO₄, filtered, and concentrated under reduced pressure to dryness. The residue was purified by flash chromatography (silica gel, 0-50% EtOAc in PE) to give 1-(tert-butyl) 2-methyl (2S,4R)-4-cyanopyrrolidine-1,2-dicarboxylate (2.80 g, yield 85.4%) as a colorless oil. LC/MS (ESI) m/z: 255 (M+H)⁺.

### Step 4: 1-(tert-butyl) 2-methyl (2S,4R)-4-carbamoylpyrrolidine-1,2-dicarboxylate

To a solution of 1-(tert-butyl) 2-methyl (2S,4R)-4-cyanopyrrolidine-1,2-dicarboxylate (2.80 g, 11.02 mmol) in MeOH (25 mL) was added K₂CO₃ (152 mg, 1.10 mmol) and H₂O₂ (25 mL, 30% wt.) at 0° C. and the mixture was stirred at room temperature for 16 hours. The mixture was diluted with water and extracted with CHCl₃/i-PrOH (3/1, v/v) twice. The combined organic layers were washed with brine, dried over anhydrous Na₂SO₄, filtered, and concentrated to give 1-(tert-butyl) 2-methyl (2S,4R)-4-carbamoylpyrrolidine-1,2-dicarboxylate (1.57 g, yield 52.3%) as a colorless oil. LC/MS (ESI) m/z: 273 (M+H)⁺.

### Step 5: 1-(tert-butyl) 2-methyl (2S,4R)-4-carbamothioylpyrrolidine-1,2-dicarboxylate

To a solution of 1-(tert-butyl) 2-methyl (2S,4R)-4-carbamoylpyrrolidine-1,2-dicarboxylate (500 mg, 1.84 mmol) in THF (10 mL) was added Lawesson's reagent (1.48 g, 3.68 mmol) at 0° C. and the mixture was stirred at 0° C. 2 hours. The mixture was diluted with EtOAc, washed with saturated aq·NaHCO₃ solution ten times, dried over anhydrous Na₂SO₄, filtered, and concentrated to dryness to give 1-(tert-butyl) 2-methyl (2S,4R)-4-carbamothioylpyrrolidine-1,2-dicarboxylate (250 mg, yield 47.3%) as a white solid, which was directly used in the next reaction without purification. LC/MS (ESI) m/z: 289 (M+H)⁺.

### Step 6: methyl (2S,4R)-4-(thiazol-2-yl)pyrrolidine-2-carboxylate

To a mixture of 1-(tert-butyl) 2-methyl (2S,4R)-4-carbamothioylpyrrolidine-1,2-dicarboxylate (250 mg, 0.89 mmol) and 2-bromo-1,1-diethoxyethane (342 mg, 1.78 mmol) in EtOH (5 mL) was added 2 N aq·HCl (0.5 mL) and the reaction mixture was stirred at 90° C. for 16 hours. The mixture was concentrated under reduced pressure to dryness to give methyl (2S,4R)-4-(thiazol-2-yl)pyrrolidine-2-carboxylate (150 mg, yield 81.5%) as a brown solid. LC/MS (ESI) m/z: 213 (M+H)⁺.

### Step 7: 1-(tert-butyl) 2-methyl (2S,4R)-4-(thiazol-2-yl)pyrrolidine-1,2-dicarboxylate

To a mixture of methyl (2S,4R)-4-(thiazol-2-yl)pyrrolidine-2-carboxylate (150 mg, 0.71 mmol) and (Boc)₂O (231 mg, 1.07 mmol) in THF (5 mL) and H₂O (3 mL) was added NaHCO₃ (594 mg, 7.10 mmol) at 0° C. and the mixture was stirred at 25° C. for 16 hours. The mixture was diluted with water and extracted with EtOAc twice. The combined organic layers were washed with brine, dried over anhydrous Na₂SO₄, filtered, and concentrated to dryness. The residue was purified by flash chromatography (silica gel, 0-5% MeOH in DCM) to give 1-(tert-butyl) 2-methyl (2S,4R)-4-(thiazol-2-yl)pyrrolidine-1,2-dicarboxylate (100 mg, yield 45.5%) as a colorless oil. LC/MS (ESI) m/z: 313 (M+H)⁺.

### Step 8: methyl (2S,4R)-4-(thiazol-2-yl)pyrrolidine-2-carboxylate hydrochloride

A solution of 1-(tert-butyl) 2-methyl (2S,4R)-4-(thiazol-2-yl)pyrrolidine-1,2-dicarboxylate (100 mg, 0.32 mmol) in HCl/1,4-dioxane (2 mL, 4M) was stirred under N₂ atmosphere at room temperature for 2 hours. The reaction mixture was concentrated under reduced pressure to dryness to give methyl (2S,4R)-4-(thiazol-2-yl)pyrrolidine-2-carboxylate hydrochloride (65 mg, yield 95.6%) as a yellow solid, which was used directly in the next reaction without further purification. LCMS (ESI) m/z: 213 (M+H)⁺.

### Step 9: methyl (2S,4R)-1-((4-(4-fluorophenoxy)benzoyl)glycyl)-4-(thiazol-2-yl)pyrrolidine-2-carboxylate

To a mixture of (4-(4-fluorophenoxy)benzoyl)glycine (93 mg, 0.32 mmol) and methyl (2S,4R)-4-(thiazol-2-yl)pyrrolidine-2-carboxylate hydrochloride (68 mg, 0.32 mmol) in DMF (3 mL) was added DIPEA (249 mg, 1.92 mmol) and T₃P (614 mg, 0.96 mmol, 50% wt. in EtOAc) at 0° C. under N₂ atmosphere and the mixture was stirred at room temperature for 1 hour. The mixture was quenched with saturated aq·NaHCO₃ solution and extracted with EtOAc twice. The combined organic layers were washed with brine, dried over anhydrous Na₂SO₄, filtered, and concentrated under reduced pressure to dryness. The residue was purified by prep-TLC (PE:EtOAc=1:1) to give methyl (2S,4R)-1-((4-(4-fluorophenoxy)benzoyl)glycyl)-4-(thiazol-2-yl)pyrrolidine-2-carboxylate (54 mg, yield 34.8%) as a brown oil. LC/MS (ESI) m/z: 248 (M+H)⁺.

### Step 10: (2S,4R)-1-((4-(4-fluorophenoxy)benzoyl)glycyl)-4-(thiazol-2-yl)pyrrolidine-2-carboxylic acid

To a solution of methyl (2S,4R)-1-((4-(4-fluorophenoxy)benzoyl)glycyl)-4-(thiazol-2-yl)pyrrolidine-2-carboxylate (54 mg, 0.11 mmol) in MeOH (3 mL) and H₂O (1 mL) was added LiOH·H₂O (8 mg, 0.17 mmol) and the reaction mixture was stirred at room temperature for 2 hours. The mixture was acidified with 1 N aq·HCl to pH ˜3 and extracted with EtOAc twice. The combined organic layers were dried over anhydrous Na₂SO₄, filtered, and concentrated under reduced pressure to dryness to give (2S,4R)-1-((4-(4-fluorophenoxy)benzoyl)glycyl)-4-(thiazol-2-yl)pyrrolidine-2-carboxylic acid (50 mg, yield 96.2%) as a yellow solid, which was used directly in the next step without further purification. LC/MS (ESI) m/z: 470 (M+H)⁺.

### Step 11: (2S,4R)—N—((R)-1-(4-carbamimidoylthiophen-2-yl)ethyl)-1-((4-(4-fluorophenoxy)benzoyl)glycyl)-4-(thiazol-2-yl)pyrrolidine-2-carboxamide (Compound 412)

To a mixture of (2S,4R)-1-((4-(4-fluorophenoxy)benzoyl)glycyl)-4-(thiazol-2-yl) pyrrolidine-2-carboxylic acid (50 mg, 0.11 mmol) and (R)-5-(1-amino-2-hydroxyethyl)thiophene-3-carboximidamide hydrochloride (37 mg, 0.22 mmol) in DMF (3 mL) was added DIPEA (83 mg, 0.66 mmol) and PyBop (70 mg, 0.13 mmol) under N₂ atmosphere and the reaction mixture was stirred at room temperature for 2 hours. The mixture was diluted with EtOAc and washed with saturated aq·NaHCO₃ solution and brine, dried over anhydrous Na₂SO₄, filtered, and concentrated under reduced pressure to dryness. The residue was purified by prep-TLC (DCM:MeOH=8:1) and further purified by prep-HPLC to give (2S,4R)—N—((R)-1-(4-carbamimidoylthiophen-2-yl)ethyl)-1-((4-(4-fluorophenoxy)benzoyl)glycyl)-4-(thiazol-2-yl)pyrrolidine-2-carboxamide (5 mg, yield 7.6%) as a white solid. ¹H NMR (400 MHz, CD₃OD) δ 8.52 (s, 1H), 8.23 (d, J=1.5 Hz, 1H), 7.86 (d, J=8.8 Hz, 2H), 7.76 (d, J=3.3 Hz, 1H), 7.57-7.49 (m, 2H), 7.16 (t, J=8.7 Hz, 2H), 7.09 (dd, J=9.1, 4.5 Hz, 2H), 7.00 (d, J=8.8 Hz, 2H), 5.29 (q, J=7.0 Hz, 1H), 4.68 (dd, J=8.4, 3.7 Hz, 1H), 4.25-4.22 (m, 2H), 4.20-3.72 (m, 3H), 2.76-2.47 (m, 2H), 1.59 (d, J=7.0 Hz, 3H). LC/MS (ESI) m/z: 621 (M+H)⁺.

### Scheme 387: Synthesis of (2S,4R)—N—((R)-1-(4-carbamimidoylthiophen-2-yl)-2-hydroxyethyl)-4-(difluoromethoxy)-1-((9,9-dimethyl-9H-fluorene-3-carbonyl)glycyl) pyrrolidine-2-carboxamide (Compound 413)

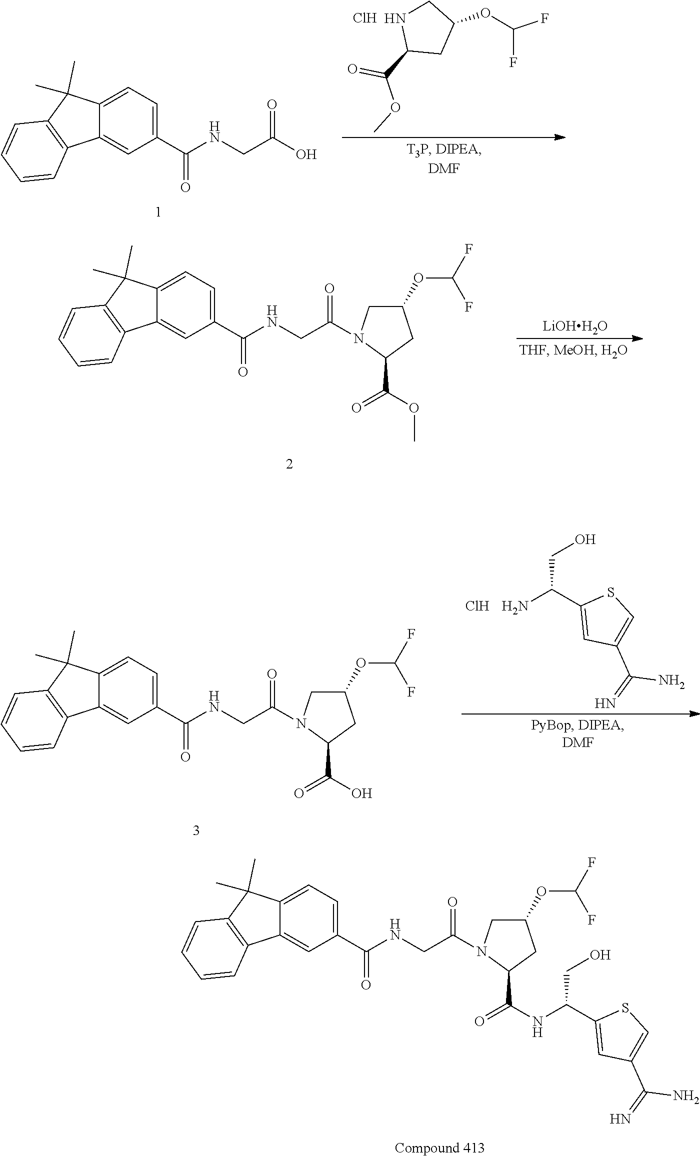

Compound 413 was prepared from (9,9-dimethyl-9H-fluorene-3-carbonyl)glycine, methyl (2S,4R)-4-(difluoromethoxy)pyrrolidine-2-carboxylate hydrochloride, and (R)-5-(1-amino-2-hydroxyethyl)thiophene-3-carboximidamide hydrochloride based on the procedures set forth in Scheme 3. ¹H NMR (400 MHz, CD₃OD) δ 8.44 (s, 1H), 8.24 (d, J=12.1 Hz, 2H), 7.85-7.77 (m, 2H), 7.52 (dd, J=19.0, 14.6 Hz, 3H), 7.36 (dd, J=5.2, 3.2 Hz, 2H), 6.51 (td, J=74.3, 16.9 Hz, 1H), 5.29 (dt, J=11.5, 6.0 Hz, 1H), 5.04 (s, 1H), 4.67 (t, J=7.6 Hz, 1H), 4.24 (dt, J=22.9, 11.5 Hz, 2H), 4.02-3.91 (m, 2H), 3.84 (tt, J=11.5, 5.7 Hz, 2H), 2.53 (ddd, J=34.6, 30.0, 12.2 Hz, 1H), 2.44-2.24 (m, 1H), 1.49 (s, 6H). LC/MS (ESI) m/z: 626 (M+H)⁺.

### Scheme 388: Synthesis of (1S,3S,5R)-5-((4-aminobutoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 414)

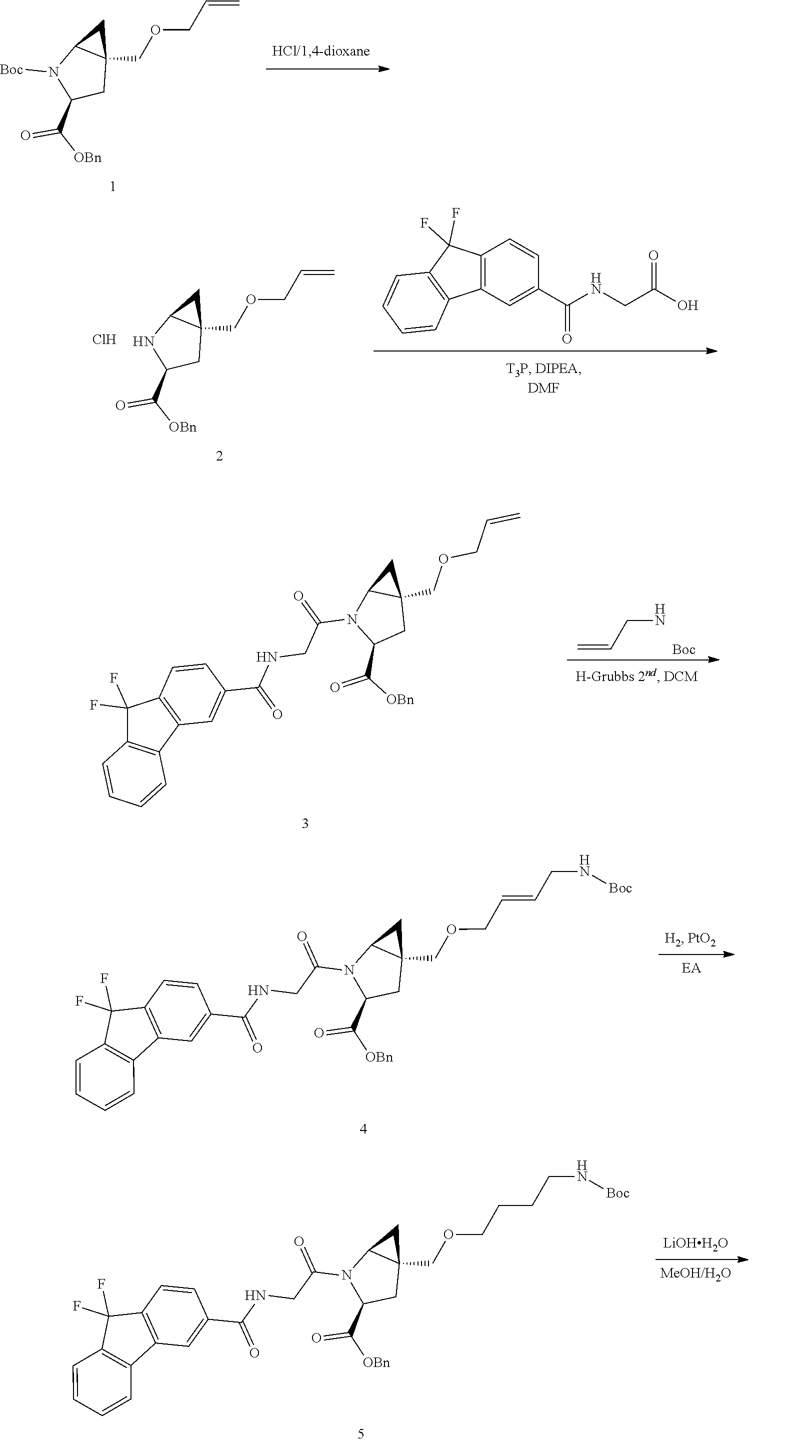

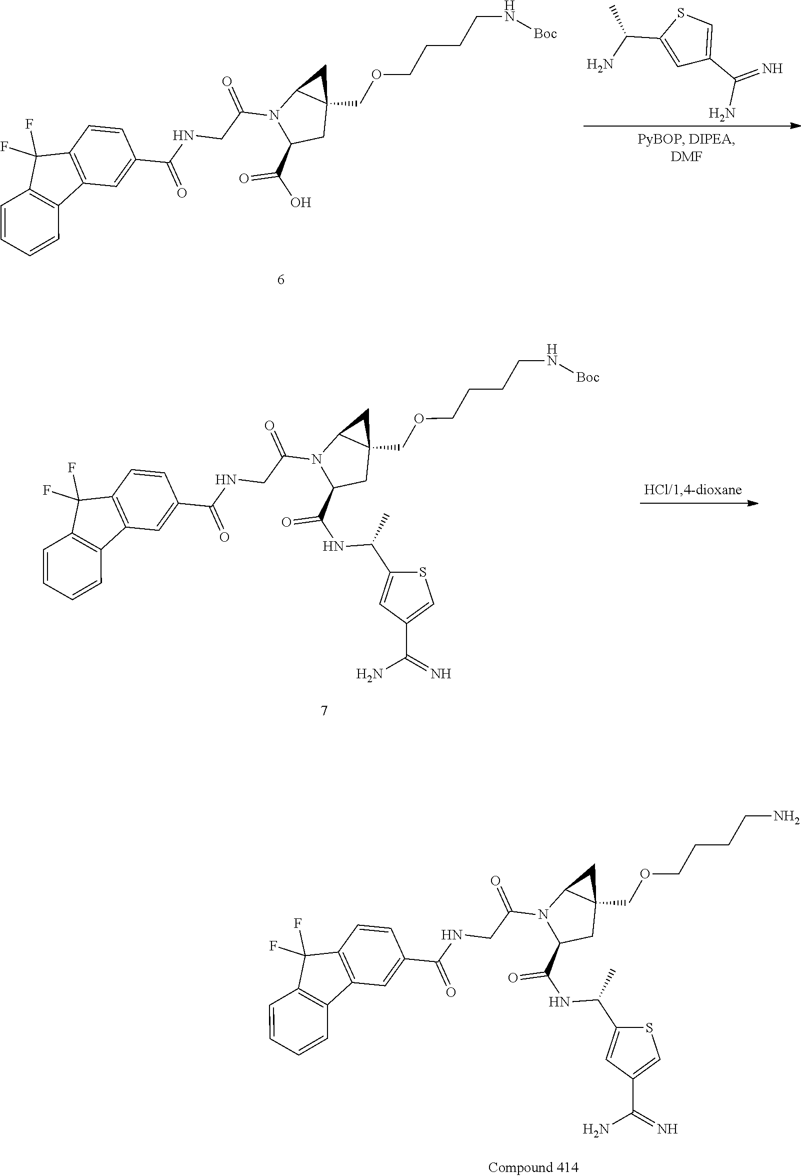 [SMILES: CC(C)(C)OC(=O)NCCCCOC[C@@]12C[C@@H]1N(C(=O)CNC(=O)c1ccc3c(c1)-c1ccccc1C3(F)F)[C@H](C(=O)O)C2 ; C[C@@H](N)c1cc(C(=N)N)cs1 ; C[C@@H](NC(=O)[C@@H]1C[C@]2(COCCCCNC(=O)OC(C)(C)C)C[C@@H]2N1C(=O)CNC(=O)c1ccc2c(c1)-c1ccccc1C2(F)F)c1cc(C(=N)N)cs1 ; C[C@@H](NC(=O)[C@@H]1C[C@]2(COCCCCN)C[C@@H]2N1C(=O)CNC(=O)c1ccc2c(c1)-c1ccccc1C2(F)F)c1cc(C(=N)N)cs1]

### Step 1: Benzyl (1S,3S,5R)-5-((allyloxy)methyl)-2-azabicyclo[3.1.0]hexane-3-carboxylate hydrochloride

A solution of 3-benzyl 2-(tert-butyl) (1 S,3S,5R)-5-((allyloxy)methyl)-2-azabicyclo[3.1.0]hexane-2,3-dicarboxylate (350 mg, 0.91 mmol) in HCl/1,4-dioxane (2 mL, 4M) was stirred under N₂ atmosphere at room temperature for 2 hours. The reaction mixture was concentrated under reduced pressure to dryness to give benzyl (1S,3S,5R)-5-((allyloxy)ethyl)-2-azabicyclo[3.1.0]hexane-3-carboxylate hydrochloride (360 mg, crude) as a white solid, which was used directly in the next reaction without further purification. LCMS (ESI) m/z: 288 (M+H)⁺.

### Step 2: Benzyl (1S,3S,5R)-5-((allyloxy)methyl)-2-((9,9-difluoro-9H-fluorene-3-carbonyl)glycyl)-2-azabicyclo[3.1.0]hexane-3-carboxylate

To a mixture of (9,9-difluoro-9H-fluorene-3-carbonyl)glycine (236 mg, 0.78 mmol) and benzyl(1 S,3S,5R)-5-((allyloxy)methyl)-2-azabicyclo[3.1.0]hexane-3-carboxylate hydrochloride (267 mg, 0.93 mmol) in DMF (5 mL) was added DIPEA (504 mg, 3.90 mmol) and T₃P (744 mg, 1.17 mmol, 50% wt. in EtOAc) at 0° C. under N₂ atmosphere and the mixture was stirred at room temperature for 1 hour. The mixture was quenched with saturated aq·NaHCO₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give benzyl (1S,3S,5R)-5-((allyloxy)methyl)-2-((9,9-difluoro-9H-fluorene-3-carbonyl)glycyl)-2-azabicyclo[3.1.0]hexane-3-carboxylate (200 mg, yield 44.7%) as a colorless oil. LCMS (ESI) (m/z): 573 (M+H)⁺.

### Step 3: Benzyl (1S,3S,5R)-5-((((E)-4-((tert-butoxycarbonyl)amino)but-2-en-1-yl)oxy)methyl)-2-((9,9-difluoro-9H-fluorene-3-carbonyl)glycyl)-2-azabicyclo[3.1.0]hexane-3-carboxylate

To a mixture of benzyl (1 S,3S,5R)-5-((allyloxy)methyl)-2-((9,9-difluoro-9H-fluorene-3-carbonyl)glycyl)-2-azabicyclo[3.1.0]hexane-3-carboxylate (200 mg, 0.35 mmol) and tert-butyl allylcarbamate (66 mg, 0.42 mmol) in anhydrous DCM (5 mL) was added Hoveyda-Grubbs 2^{nd} (44 mg, 0.07 mmol) at room temperature under N₂ atmosphere, the reaction mixture was degassed under N₂ atmosphere for three times and stirred at room temperature overnight. The mixture was concentrated under reduced pressure to dryness. The residue was purified by flash chromatography (silica gel, 0-60% EtOAc in PE) and further purified by prep-TLC (DCM:MeOH=20:1) to give benzyl (1S,3S,5R)-5-((((E)-4-((tert-butoxycarbonyl)amino)but-2-en-1-yl)oxy)methyl)-2-((9,9-difluoro-9H-fluorene-3-carbonyl)glycyl)-2-azabicyclo[3.1.0]hexane-3-carboxylate (40 mg, yield 16.3%) as a brown oil. LC/MS (ESI) m/z: 702 (M+H)⁺.

### Step 4: Benzyl (1S,3S,5R)-5-((4-((tert-butoxycarbonyl)amino)butoxy)methyl)-2-((9,9-difluoro-9H-fluorene-3-carbonyl)glycyl)-2-azabicyclo[3.1.0]hexane-3-carboxylate

To a solution of benzyl (1S,3S,5R)-5-((((E)-4-((tert-butoxycarbonyl)amino)but-2-en-1-yl)oxy)methyl)-2-((9,9-difluoro-9H-fluorene-3-carbonyl)glycyl)-2-azabicyclo[3.1.0]hexane-3-carboxylate (40 mg, 0.057 mmol) in EtOAc (2 mL) was added PtO₂ (13 mg, 0.057 mmol), the mixture was degassed under N₂ atmosphere for three times and stirred under a H₂ balloon at room temperature for 10 mins. The mixture was filtered, and the filtrate was concentrated to dryness to give benzyl (1 S,3S,5R)-5-((4-((tert-butoxycarbonyl)amino)butoxy)methyl)-2-((9,9-difluoro-9H-fluorene-3-carbonyl)glycyl)-2-azabicyclo[3.1.0]hexane-3-carboxylate (40 mg, yield 99.7%) as a brown oil, which was used directly in the next step without further purification. LC/MS (ESI) m/z: 704 (M+H)⁺.

### Step 5: (1S,3S,5R)-5-((4-((tert-butoxycarbonyl)amino)butoxy)methyl)-2-((9,9-difluoro-9H-fluorene-3-carbonyl)glycyl)-2-azabicyclo[3.1.0]hexane-3-carboxylic acid

To a solution of benzyl (1S,3S,5R)-5-((4-((tert-butoxycarbonyl)amino)butoxy)methyl)-2-((9,9-difluoro-9H-fluorene-3-carbonyl)glycyl)-2-azabicyclo[3.1.0]hexane-3-carboxylate (40 mg, 0.057 mmol) in THF/MeOH/H₂O (1.5 mL, 4/1/1) was added LiOH·H₂O (7 mg, 0.17 mmol) under N₂ atmosphere and the reaction mixture was stirred at room temperature for 2 hours. The mixture was acidified with 1 N aq·HCl to pH˜3 and extracted with EtOAc twice. The combined organic layers were dried over anhydrous Na₂SO₄, filtered, and concentrated under reduced pressure to give (1 S,3S,5R)-5-((4-((tert-butoxycarbonyl)amino)butoxy)methyl)-2-((9,9-difluoro-9H-fluorene-3-carbonyl)glycyl)-2-azabicyclo[3.1.0]hexane-3-carboxylic acid (33 mg, yield 94.3%) as a white solid, which was used directly in the next reaction without further purification. LC/MS (ESI) m/z: 614 (M+H)⁺.

### Step 6: Tert-butyl (4-(((1S,3S,5R)-3-(((R)-1-(4-carbamimidoylthiophen-2-yl)ethyl) carbamoyl)-2-((9,9-difluoro-9H-fluorene-3-carbonyl)glycyl)-2-azabicyclo[3.1.0]hexan-5-yl)methoxy)butyl)carbamate

To a mixture of (1S,3S,5R)-5-((4-((tert-butoxycarbonyl)amino)butoxy)methyl)-2-((9,9-difluoro-9H-fluorene-3-carbonyl)glycyl)-2-azabicyclo[3.1.0]hexane-3-carboxylic acid (33 mg, 0.054 mmol) and (R)-5-(1-aminoethyl)thiophene-3-carboximidamide hydrochloride (22 mg, 0.11 mmol) in DMF (2 mL) was added DIPEA (35 mg, 0.27 mmol) and PyBop (37 mg, 0.072 mmol) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=10:1) to give tert-butyl (4-(((1S,3S,5R)-3-(((R)-1-(4-carbamimidoylthiophen-2-yl)ethyl)carbamoyl)-2-((9,9-difluoro-9H-fluorene-3-carbonyl)glycyl)-2-azabicyclo[3.1.0]hexan-5-yl)methoxy)butyl)carbamate (17 mg, yield 38.6%) as a white solid. LC/MS (ESI) m/z: 765 (M+H)⁺.

### Step 7: (1S,3S,5R)-5-((4-aminobutoxy)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 414)

A solution of tert-butyl (4-(((1 S,3S,5R)-3-(((R)-1-(4-carbamimidoylthiophen-2-yl)ethyl) carbamoyl)-2-((9,9-difluoro-9H-fluorene-3-carbonyl)glycyl)-2-azabicyclo[3.1.0]hexan-5-yl)methoxy)butyl)carbamate (17 mg, 0.022 mmol) in HCl/1,4-dioxane (0.5 mL, 4M) was stirred under N₂ atmosphere at room temperature for 2 hours. The reaction mixture was concentrated under reduced pressure to dryness. The residue was purified by prep-HPLC to give Compound 414 (4.0 mg, yield 27.4%) as a white solid. ¹H NMR (400 MHz, CD₃OD) δ 8.52 (s, 2H), 8.23 (t, J=7.3 Hz, 1H), 8.19 (s, 1H), 7.88 (dd, J=7.8, 1.5 Hz, 1H), 7.77-7.72 (m, 2H), 7.66 (d, J=7.3 Hz, 1H), 7.58 (d, J=7.0 Hz, 1H), 7.51 (s, 1H), 7.45 (d, J=7.6 Hz, 1H), 5.24 (d, J=6.9 Hz, 1H), 4.52 (d, J=16.6 Hz, 1H), 4.28 (d, J=16.5 Hz, 1H), 3.63-3.47 (m, 6H), 3.00-2.95 (m, 2H), 2.69 (t, J=12.0 Hz, 1H), 2.13 (dd, J=13.4, 3.6 Hz, 1H), 1.79-1.73 (m, 2H), 1.72-1.67 (m, 2H), 1.60 (t, J=13.3 Hz, 3H), 1.38 (s, 1H), 1.03 (d, J=5.4 Hz, 1H). LCMS (ESI) (m/z): 665 (M+H)⁺.

### Scheme 389: Synthesis of (1S,3S,5R)—N—((R)-1-(4-carbamimidoylthiophen-2-yl)ethyl)-2-((9,9-difluoro-9H-fluorene-3-carbonyl)glycyl)-5-((4-(dimethylamino)butoxy)methyl)-2-azabicyclo[3.1.0]hexane-3-carboxamide (Compound 415)

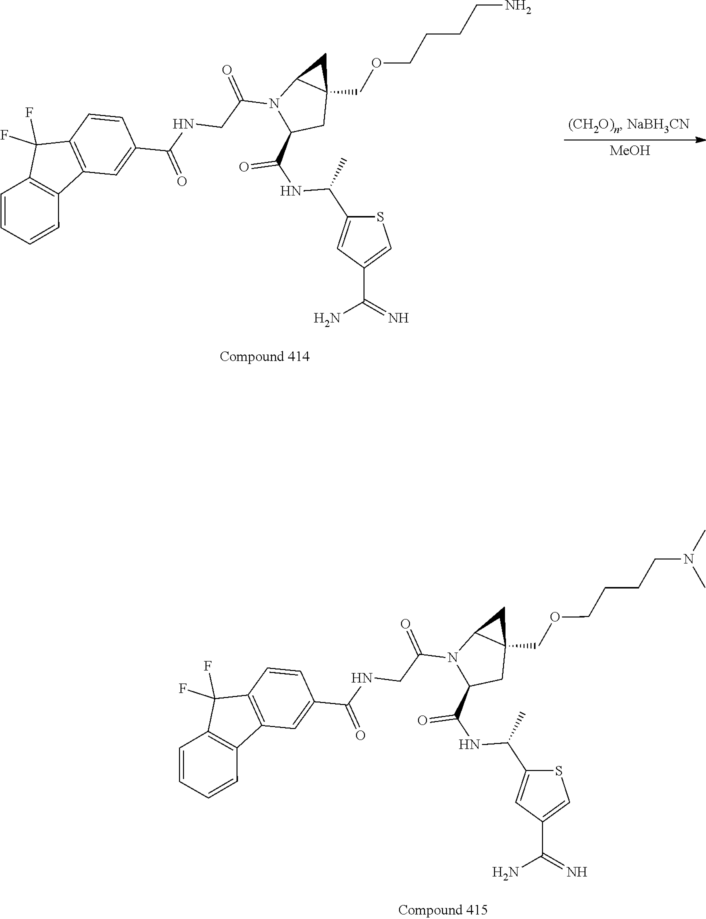 [SMILES: "MeOH" ; C[C@@H](NC(=O)[C@@H]1C[C@]2(COCCCCN)C[C@@H]2N1C(=O)CNC(=O)c1ccc2c(c1)-c1ccccc1C2(F)F)c1cc(C(=N)N)cs1 ; C[C@@H](NC(=O)[C@@H]1C[C@]2(COCCCCN(C)C)C[C@@H]2N1C(=O)CNC(=O)c1ccc2c(c1)-c1ccccc1C2(F)F)c1cc(C(=N)N)cs1]

To solution of Compound 414 (2 mg, 0.003 mmol) in MeOH (1 mL) was added (CH₂O)ₙ (2 mg) and NaBH₃CN (2 mg, 0.032 mmol) and the reaction mixture was stirred under N₂ atmosphere at room temperature overnight. The reaction mixture was concentrated under reduced pressure to dryness. The residue was purified by prep-HPLC to give Compound 415 (0.5 mg, yield 24.1%) as a white solid. ¹H NMR (400 MHz, CD₃OD) δ 8.53 (s, 1H), 8.25-8.15 (m, 2H), 7.88 (dd, J=7.8, 1.3 Hz, 1H), 7.74 (dd, J=12.2, 7.8 Hz, 2H), 7.66 (d, J=7.4 Hz, 1H), 7.58 (t, J=7.5 Hz, 1H), 7.52 (d, J=8.3 Hz, 1H), 7.46 (t, J=7.4 Hz, 1H), 5.34-5.21 (m, 1H), 4.88 (d, J=3.9 Hz, 1H), 4.51 (d, J=16.6 Hz, 1H), 4.29 (d, J=16.5 Hz, 1H), 3.60 (dd, J=11.6, 6.4 Hz, 2H), 3.55-3.47 (m, 3H), 2.90 (dd, J=14.8, 7.1 Hz, 2H), 2.69 (s, 6H), 2.18-2.09 (m, 1H), 1.74 (dd, J=14.6, 6.9 Hz, 2H), 1.68-1.63 (m, 2H), 1.60 (t, J=9.0 Hz, 3H), 1.38-1.30 (m, 2H), 1.02 (t, J=6.0 Hz, 1H). LCMS (ESI) (m/z): 693 (M+H)⁺.

### Scheme 390: (S)—N—((R)-1-(4-carbamimidoylthiophen-2-yl)ethyl)-1-((9,9-difluoro-9H-fluorene-3-carbonyl)glycyl)-4-(oxazol-2-yl)pyrrolidine-2-carboxamide (Compound 416)

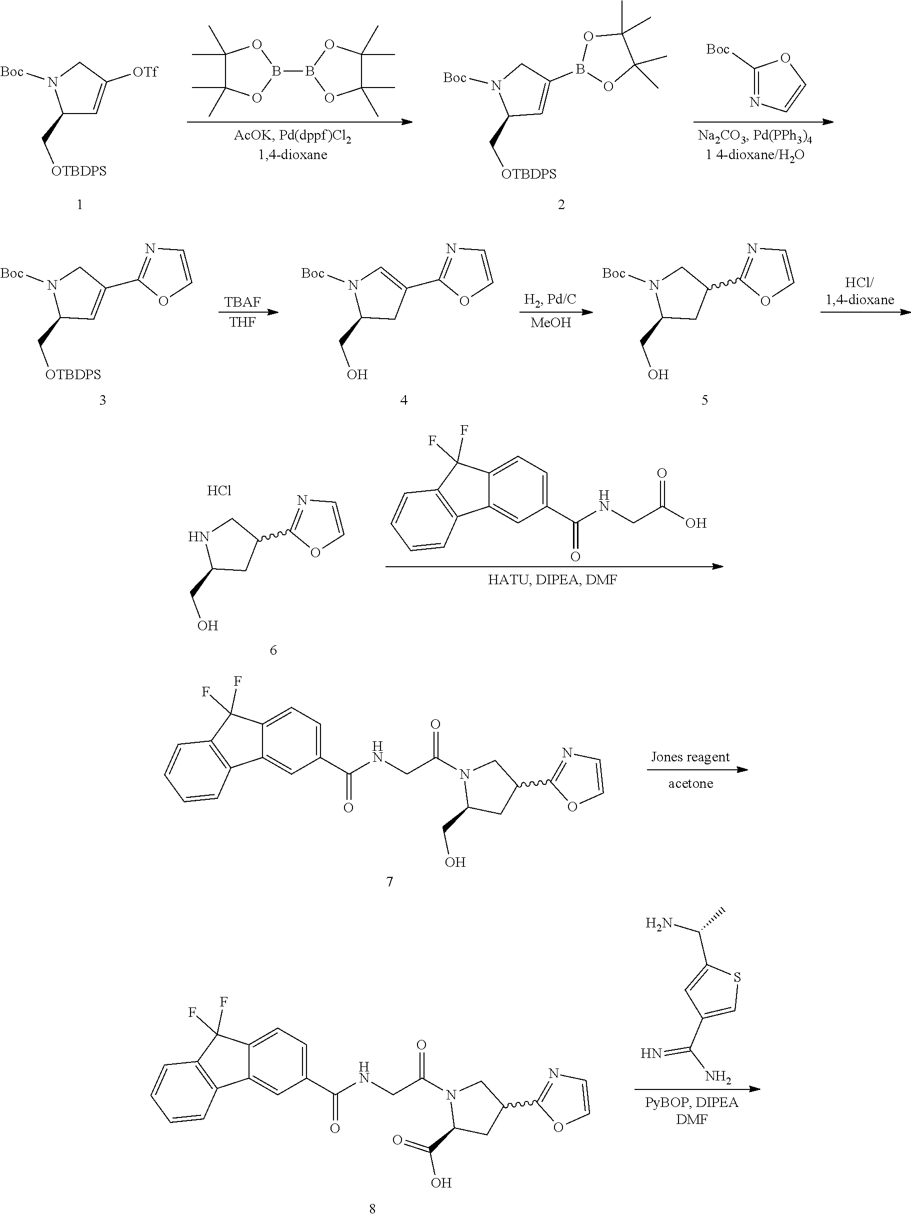

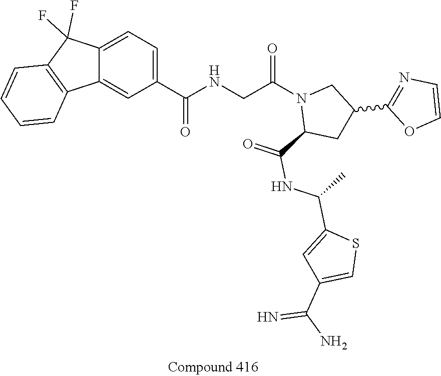 [SMILES: C[C@@H](NC(=O)[C@@H]1CC(c2ncco2)CN1C(=O)CNC(=O)c1ccc2c(c1)-c1ccccc1C2(F)F)c1cc(C(=N)N)cs1]

Compound 416 was prepared from tert-butyl (S)-2-(((tert-butyldiphenylsilyl)oxy)methyl)-4-(((trifluoromethyl)sulfonyl)oxy)-2,5-dihydro-1H-pyrrole-1-carboxylate, 2-bromooxazole, (9,9-difluoro-9H-fluorene-3-carbonyl)glycine, and (R)-5-(1-aminoethyl) thiophene-3-carboximidamide based on the procedures set forth in Scheme 339. ¹H NMR (400 MHz, CD₃OD) δ 8.51 (s, 1H), 8.19 (dd, J=10.0, 4.4 Hz, 2H), 7.89-7.85 (m, 2H), 7.74 (dd, J=16.8, 7.7 Hz, 2H), 7.65 (d, J=7.4 Hz, 1H), 7.58 (t, J=7.5 Hz, 1H), 7.51 (d, J=5.6 Hz, 1H), 7.45 (t, J=7.4 Hz, 1H), 7.10 (d, J=10.3 Hz, 1H), 5.25 (q, J=6.6 Hz, 1H), 4.57 (d, J=7.0 Hz, 1H), 4.38 (t, J=15.4 Hz, 1H), 4.30 (d, J=7.7 Hz, 1H), 4.20 (d, J=16.6 Hz, 1H), 3.97 (t, J=9.7 Hz, 1H), 3.83 (t, J=8.6 Hz, 1H), 2.84-2.75 (m, 1H), 2.42-2.28 (m, 1H), 1.60 (t, J=14.1 Hz, 3H). LC/MS (ESI) m/z: 619 (M+H)⁺.

### Scheme 391: Synthesis of (2S,4R)—N—((R)-1-(4-carbamimidoylthiophen-2-yl)ethyl)-4-fluoro-4-(fluoromethyl)-1-((9-methyl-9H-fluorene-3-carbonyl)glycyl)pyrrolidine-2-carboxamide (Compound 417)

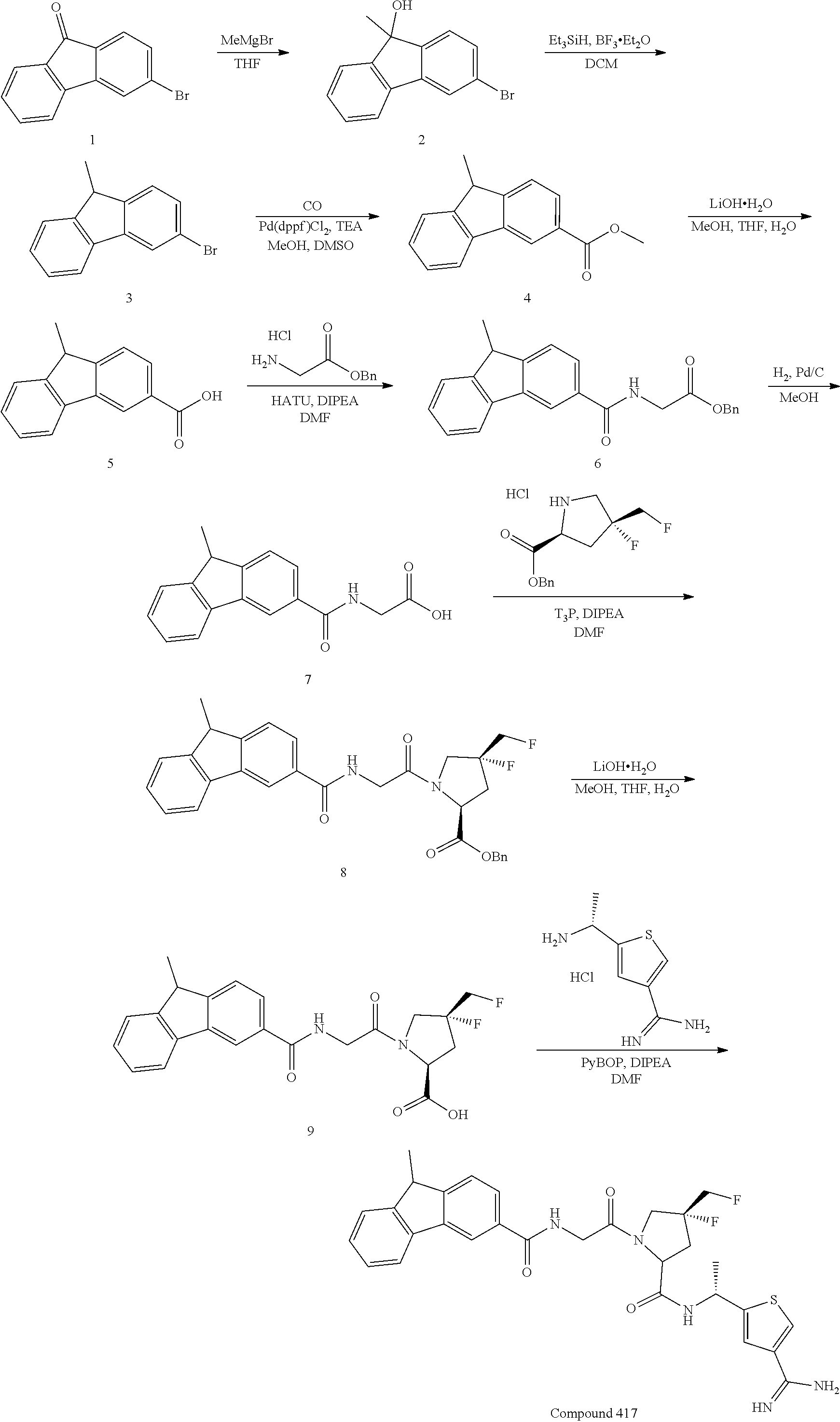

### Step 1: 3-Bromo-9-methyl-9H-fluoren-9-ol (2)

To a solution of 3-bromo-9H-fluoren-9-one (2.0 g, 7.70 mmol) in THF (20 mL) was added MeMgBr (25.7 mL, 77.2 mmol, 3M in THF) drop-wisely at 0° C. and the mixture was stirred at room temperature for 3 hours. The mixture was quenched with ice-cooled saturated aq·NH₄Cl solution and extracted with EtOAc twice. The combined organic layers were washed with brine, dried over anhydrous Na₂SO₄, filtered, and concentrated to dryness. The residue was purified by flash chromatography (silica gel, 0-10% EtOAc in PE) to give 3-bromo-9-methyl-9H-fluoren-9-ol (2.0 g, yield 94.3%) as a white solid. LC/MS (ESI) m/z: 257 (M−OH)⁺.

### Step 2: 3-Bromo-9-methyl-9H-fluorene (3)

To a solution of 3-bromo-9-methyl-9H-fluoren-9-ol (1.0 g, 3.65 mmol) in DCM (10 mL) was added Et₃SiH (2.1 g, 18.25 mmol) followed by BF₃·Et₂O (2.6 g, 18.25 mmol) at 0° C. and the mixture was stirred at room temperature for 3 hours. The mixture was quenched with ice-cooled saturated aq·NH₄Cl solution and extracted with EtOAc twice. The combined organic layers were washed with brine, dried over anhydrous Na₂SO₄, filtered, and concentrated to dryness. The residue was purified by flash chromatography (silica gel, 0-10% EtOAc in PE) to give 3-bromo-9-methyl-9H-fluorene (560 mg, yield 59.5%) as a colorless oil.

### Step 3: Methyl 9-methyl-9H-fluorene-3-carboxylate (4)

To a mixture of 3-bromo-9-methyl-9H-fluorene (500 mg, 1.94 mmol) in MeOH (3 mL) and DMSO (3 mL) was added TEA (587 mg, 5.81 mmol) and Pd(dppt)Cl₂ (284 mg, 0.39 mmol) under N₂ atmosphere. The mixture was degassed under N₂ atmosphere for three times and stirred under CO balloon at 85° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-10% EtOAc in PE) to give methyl 9-methyl-9H-fluorene-3-carboxylate (205 mg, yield 44.4%) as a colorless oil. LC/MS (ESI) m/z: 239 (M+H)⁺.

### Step 4: 9-Methyl-9H-fluorene-3-carboxylic acid (5)

To a solution of methyl 9-methyl-9H-fluorene-3-carboxylate (200 mg, 0.84 mmol) in THF/MeOH/H₂O (4 mL, 2/1/1) was added LiOH H₂O (106 mg, 2.52 mmol) and the mixture was stirred at 30° C. for 1 hour. The mixture was acidified with 1 N aq·HCl to pH ˜3 and extracted with EtOAc twice. The combined organic layers were washed with brine, dried over anhydrous Na₂SO₄, filtered, and concentrated under reduced pressure to dryness to give 9-methyl-9H-fluorene-3-carboxylic acid (150 mg, yield 79.8%) as a white solid, which was used directly in the next reaction without further purification. LC/MS (ESI) m/z: 223 (M−H)⁻.

### Step 5: Benzyl (9-methyl-9H-fluorene-3-carbonyl)glycinate (6)

To a mixture of 9-methyl-9H-fluorene-3-carboxylic acid (150 mg, 0.67 mmol) and benzyl glycinate hydrochloride (203 mg, 1.00 mmol) in DMF (2 mL) was added DIPEA (432 mg, 3.35 mmol) and HATU (382 mg, 1.00 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give benzyl (9-methyl-9H-fluorene-3-carbonyl)glycinate (150 mg, yield 60.4%) as a colorless oil. LC/MS (ESI) m/z: 372 (M+H)⁺.

### Step 6: (9-Methyl-9H-fluorene-3-carbonyl)glycine (7)

To a solution of benzyl (9-methyl-9H-fluorene-3-carbonyl)glycinate (150 mg, 0.40 mmol) in MeOH (4 mL) was added Pd/C (20 mg, 10% wt.), the mixture was degassed under N₂ atmosphere for three times and stirred under a H₂ balloon at room temperature for 2 hours. The mixture was filtered, and the filtrate was concentrated to dryness to give (9-methyl-9H-fluorene-3-carbonyl)glycine (110 mg, yield 96.8%) as a white solid, which was used directly in the next reaction without further purification. LC/MS (ESI) m/z: 282 (M+H)⁺.

### Step 7: Benzyl (2S,4R)-4-fluoro-4-(fluoromethyl)-1-((9-methyl-9H-fluorene-3-carbonyl)glycyl)pyrrolidine-2-carboxylate (6)

To a mixture of (9-methyl-9H-fluorene-3-carbonyl)glycine (35 mg, 0.12 mmol) and benzyl (2S,4R)-4-fluoro-4-(fluoromethyl)pyrrolidine-2-carboxylate hydrochloride (41 mg, 0.16 mmol) in DMF (2 mL) was added DIPEA (80 mg, 0.62 mmol) and T₃P (119 mg, 0.19 mmol, 50% wt. in EtOAc) under N₂ atmosphere at 0° C. and the mixture was stirred at room temperature for 1 hour. The mixture was quenched with saturated aq·NaHCO₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-80% EtOAc in PE) to give benzyl (2S,4R)-4-fluoro-4-(fluoromethyl)-1-((9-methyl-9H-fluorene-3-carbonyl)glycyl)pyrrolidine-2-carboxylate (50 mg, yield 77.5%) as a white solid. LCMS (ESI) (m/z): 519 (M+H)⁺.

### Step 6: (2S,4R)-4-fluoro-4-(fluoromethyl)-1-((9-methyl-9H-fluorene-3-carbonyl) glycyl)pyrrolidine-2-carboxylic acid (9)

To a solution of benzyl (2S,4R)-4-fluoro-4-(fluoromethyl)-1-((9-methyl-9H-fluorene-3-carbonyl)glycyl)pyrrolidine-2-carboxylate (50 mg, 0.10 mmol) in THF/MeOH/H₂O (2 mL, 2/1/1) was added LiOH·H₂ (12 mg, 0.30 mmol) and the mixture was stirred at room temperature for 0.5 hour. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (2S,4R)-4-fluoro-4-(fluoromethyl)-1-((9-methyl-9H-fluorene-3-carbonyl)glycyl)pyrrolidine-2-carboxylic acid (30 mg, yield 72.6%) as a white solid, which was used directly in the next step without further purification. LC/MS (ESI) m/z: 429 (M+H)⁺.

### Step 9: (2S,4R)—N—((R)-1-(4-carbamimidoylthiophen-2-yl)ethyl)-4-fluoro-4-(fluoromethyl)-1-((9-methyl-9H-fluorene-3-carbonyl)glycyl)pyrrolidine-2-carboxamide (Compound 417)

To a mixture of (2S,4R)-4-fluoro-4-(fluoromethyl)-1-((9-methyl-9H-fluorene-3-carbonyl)glycyl)pyrrolidine-2-carboxylic acid (30 mg, 0.07 mmol) and (R)-5-(1-aminoethyl)thiophene-3-carboximidamide hydrochloride (15 mg, 0.09 mmol) in DMF (2 mL) was added DIPEA (45 mg, 0.35 mmol) and PyBOP (55 mg, 0.11 mmol) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=10:1) and further purified by prep-HPLC to give Compound 417 (7.0 mg, yield 17.2%) as a white solid. H NMR (400 MHz, CD₃OD) δ 8.49 (s, 1H), 8.29-8.25 (m, 1H), 8.20 (s, 1H), 7.82 (d, J=7.7 Hz, 2H), 7.63 (d, J=7.9 Hz, 1H), 7.56 (d, J=10.6 Hz, 2H), 7.37 (t, J=6.1 Hz, 2H), 5.39-5.25 (m, 1H), 4.76 (dd, J=11.1, 9.1 Hz, 1H), 4.70-4.61 (m, 2H), 4.35 (d, J=16.7 Hz, 1H), 4.20-4.12 (m, 2H), 3.96 (dd, J=23.3, 8.9 Hz, 2H), 2.69-2.56 (m, 1H), 2.23 (ddd, 1H), 1.63 (dd, J=7.0 Hz, 3H), 1.52 (d, J=7.4 Hz, 3H). LC/MS (ESI) m/z: 580 (M+H)⁺.

### Scheme 392: Synthesis of (2S,4R)—N—((R)-1-(4-carbamimidoylthiophen-2-yl)ethyl)-4-fluoro-4-(methoxymethyl)-1-((9-methyl-9H-fluorene-3-carbonyl)glycyl)pyrrolidine-2-carboxamide (Compound 418)

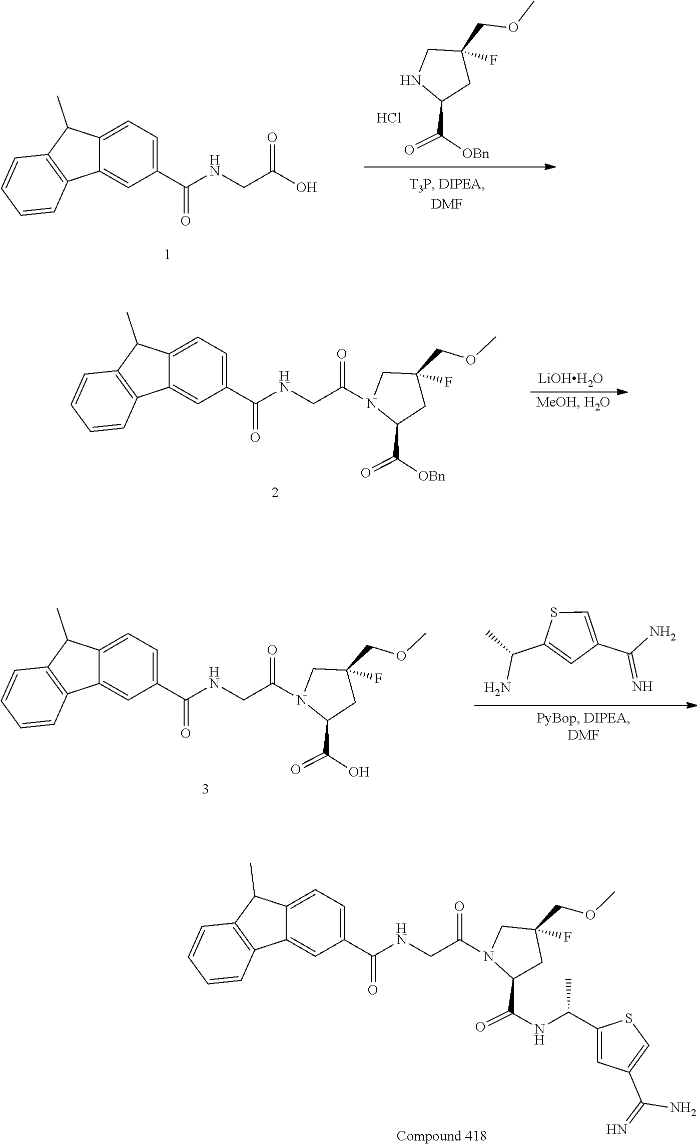

Compound 418 was prepared from (9-methyl-9H-fluorene-3-carbonyl)glycine, benzyl (2S,4R)-4-fluoro-4-(methoxymethyl)pyrrolidine-2-carboxylate hydrochloride, and (R)-5-(1-aminoethyl)thiophene-3-carboximidamide based on the procedures set forth in Scheme 3. ¹H NMR (400 MHz, CD₃OD) δ 8.50 (s, 1H), 8.29-8.25 (m, 1H), 8.20 (s, 1H), 7.82 (d, J=6.5 Hz, 2H), 7.64 (d, J=7.8 Hz, 1H), 7.57 (d, J=8.6 Hz, 2H), 7.37 (t, J=6.2 Hz, 2H), 5.28 (q, J=6.8 Hz, 1H), 4.67-4.61 (m, 1H), 4.34 (d, J=16.7 Hz, 1H), 4.17 (d, J=17.0 Hz, 1H), 4.09-3.97 (m, 2H), 3.96-3.84 (m, 1H), 3.73 (d, J=4.4 Hz, 1H), 3.69 (d, J=6.8 Hz, 1H), 3.43 (d, J=4.6 Hz, 3H), 2.62-2.49 (m, 1H), 2.20 (ddd, J=36.8, 14.3, 9.6 Hz, 1H), 1.61 (t, J=16.1 Hz, 3H), 1.52 (d, J=7.5 Hz, 3H). LC/MS (ESI) m/z: 592 (M+H)⁺.

### Scheme 393: Synthesis of (2S,4R)—N—((R)-1-(4-carbamimidoylthiophen-2-yl)ethyl)-1-((9-methyl-9H-fluorene-3-carbonyl)glycyl)-4-(methylsulfonyl)pyrrolidine-2-carboxamide (Compound 419)

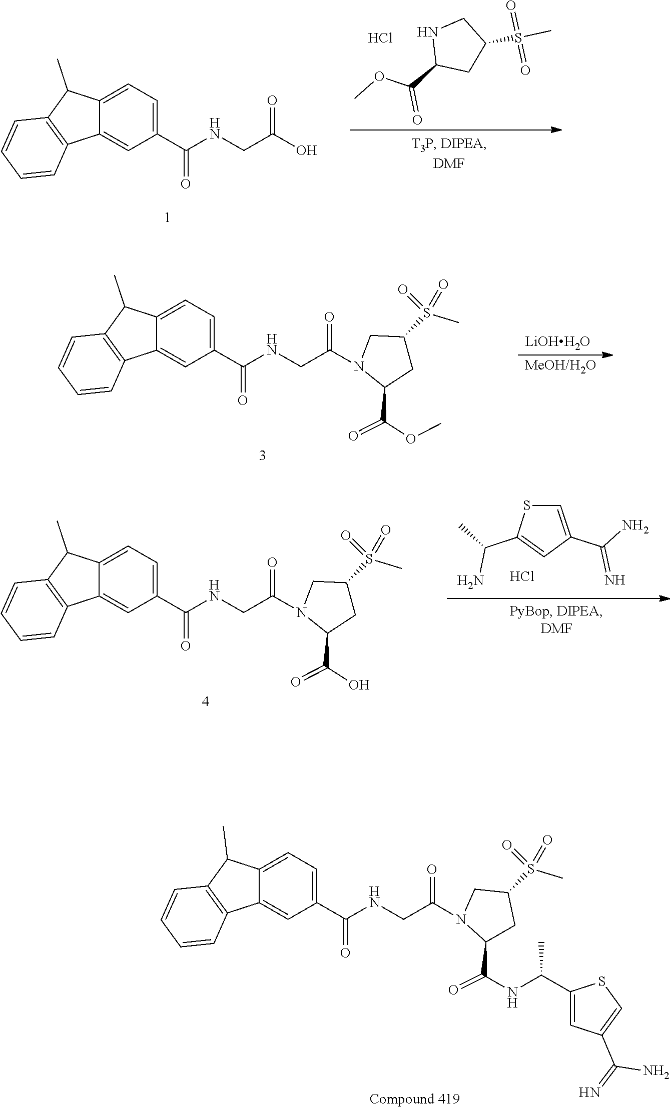

Compound 419 was prepared from (9-methyl-9H-fluorene-3-carbonyl)glycine, methyl (2S,4R)-4-(methylsulfonyl)pyrrolidine-2-carboxylate hydrochloride, and (R)-5-(1-aminoethyl)thiophene-3-carboximidamide hydrochloride based on the procedures set forth in Scheme 3. ¹H NMR (400 MHz, CD₃OD) 8.52 (s, 1H), 8.31-8.15 (m, 2H), 7.90-7.76 (m, 2H), 7.63 (d, J=7.9 Hz, 1H), 7.59-7.47 (m, 2H), 7.41-7.33 (m, 2H), 5.36-5.23 (m, 1H), 4.82-4.77 (m, 1H), 4.73 (dd, J=8.7, 4.9 Hz, 1H), 4.18 (dt, J=23.5, 11.1 Hz, 3H), 4.00 (d, J=7.8 Hz, 1H), 3.07 (d, J=18.4 Hz, 3H), 2.93-2.72 (m, 1H), 2.58-2.39 (m, 1H), 1.63 (dd, J=31.3, 7.0 Hz, 3H), 1.52 (d, J=7.4 Hz, 3H). LC/MS (ESI) m/z: 608 (M+H)⁺.

### Scheme 394: Synthesis of (1S,3S,5S)-5-((1,2,4-oxadiazol-5-yl)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 420)

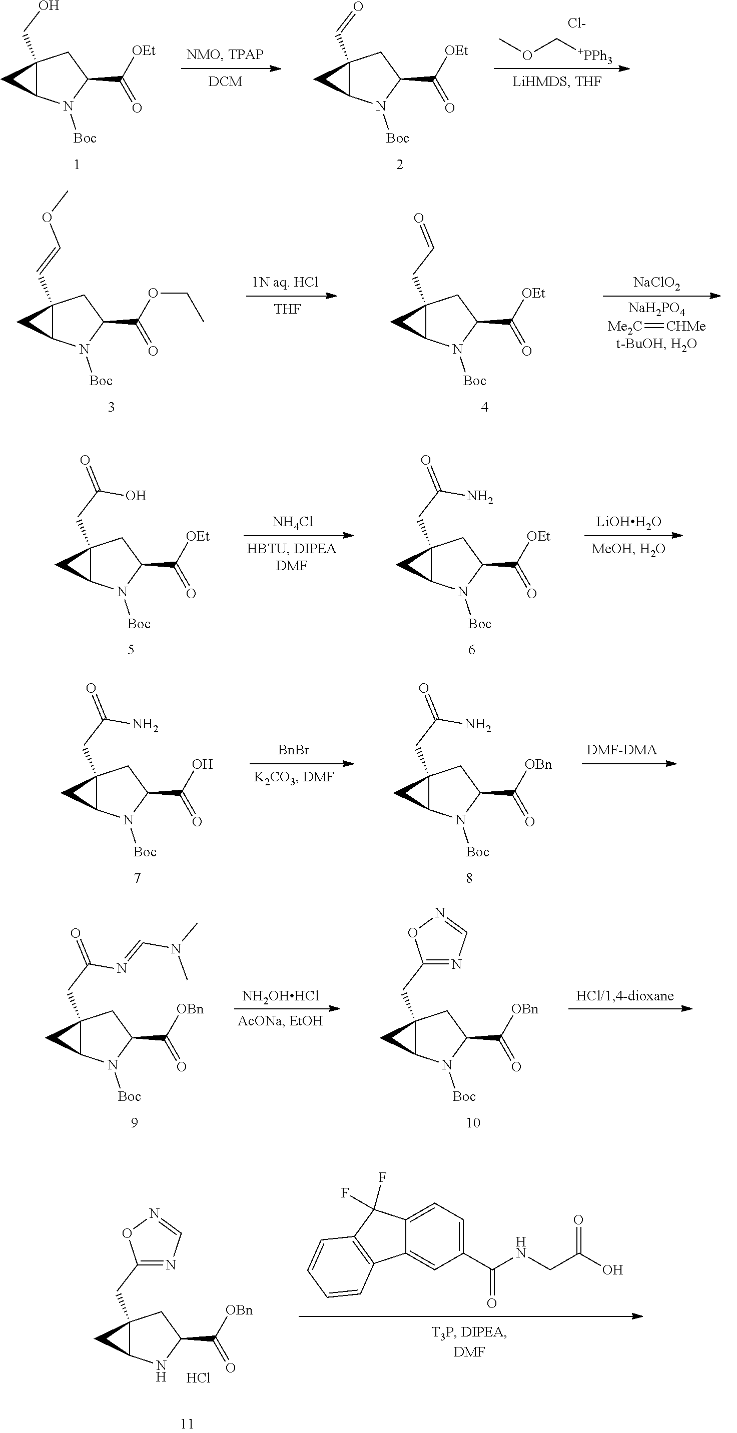

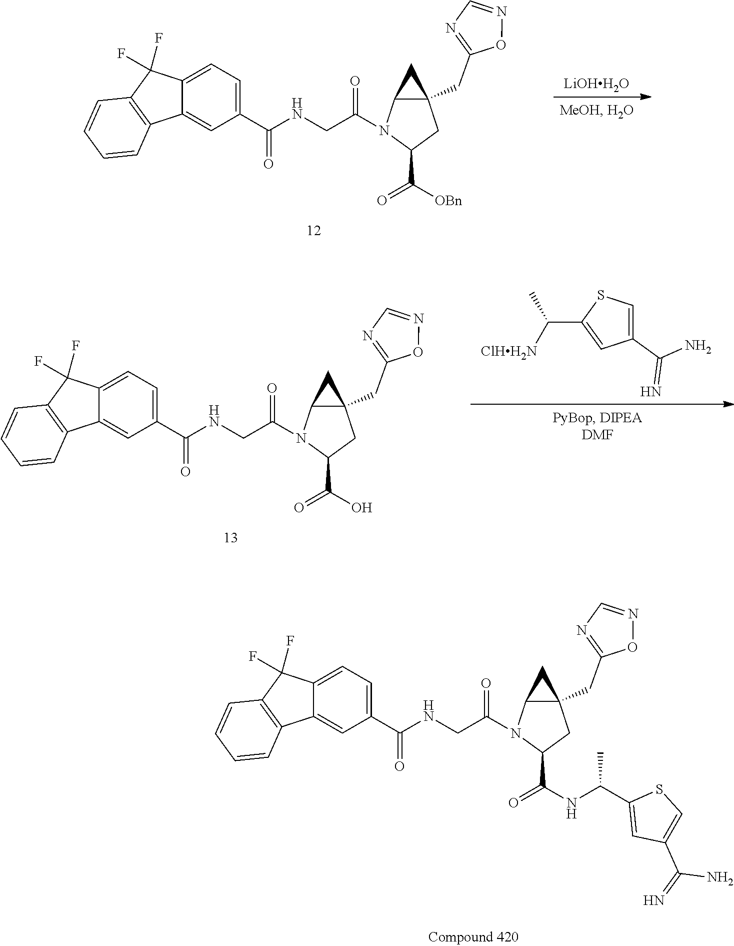

### Step 1: 2-(Tert-butyl) 3-ethyl (1S,3S,5R)-5-formyl-2-azabicyclo[3.1.0]hexane-2,3-dicarboxylate

To a mixture of 2-(tert-butyl) 3-ethyl (1S,3S,5R)-5-(hydroxymethyl)-2-azabicyclo [3.1.0]hexane-2,3-dicarboxylate (500 mg, 1.75 mmol) and TPAP (62 mg, 0.18 mmol) in DCM (10 mL) was added NMO (308 mg, 2.63 mmol) at 0° C. and the reaction mixture was stirred at 25° C. 1 hour. Silica gel was added, and the mixture was stirred at 25° C. for 1 hour. The mixture was filtered, and the filtrate was concentrated under reduced pressure to dryness to give 2-(tert-butyl) 3-ethyl (1S,3S,5R)-5-formyl-2-azabicyclo[3.1.0]hexane-2,3-dicarboxylate (383 mg, yield 77.2%) as black oil. LC/MS (ESI) m/z: 284 (M+H)⁺.

### Step 2: 2-(Tert-butyl) 3-ethyl (1S,3S,5S)-5-((E)-2-methoxyvinyl)-2-azabicyclo[3.1.0]hexane-2,3-dicarboxylate

To a solution of (methoxymethyl)triphenylphosphonium chloride (2.12 g, 6.18 mmol) in THF (6 mL) was added LiHMDS (5.7 mL, 5.68 mmol, 1 N in THF) drop-wisely at −5° C. and the mixture was stirred at −5° C. for 0.5 hour. Then a solution of 2-(tert-butyl) 3-ethyl (1S,3S,5S)-5-((E)-2-methoxyvinyl)-2-azabicyclo[3.1.0]hexane-2,3-dicarboxylate (700 mg, 2.47 mmol in THE (3 mL) was added and the resulting mixture was stirred at 25° C. for 1 hour. The mixture was quenched with saturated aq·NH₄Cl solution and extracted with EtOAc twice. The combined organic layers were washed with brine, dried over anhydrous Na₂SO₄, filtered, and concentrated to dryness. The residue was purified by flash chromatography (silica gel, 0-30% EtOAc in PE) to give 2-(tert-butyl) 3-ethyl (1S,3S,5S)-5-((E)-2-methoxyvinyl)-2-azabicyclo[3.1.0]hexane-2,3-dicarboxylate (488 mg, yield 63.5%) as a yellow oil. LC/MS (ESI) m/z: 312 (M+H)⁺.

### Step 3: 2-(Tert-butyl) 3-ethyl (1S,3S,5S)-5-(2-oxoethyl)-2-azabicyclo[3.1.0]hexane-2,3-dicarboxylate

To a solution of 2-(tert-butyl) 3-ethyl (1 S,3S,5S)-5-((E)-2-methoxyvinyl)-2-azabicyclo[3.1.0]hexane-2,3-dicarboxylate (488 mg, 1.57 mmol) in THF (3 mL) was added 1 N aq·HCl (3 mL) at 0° C. and the mixture was stirred at 25° C. for 16 hours. The mixture was diluted with EtOAc, washed with saturated aq·NaHCO₃ solution, dried over anhydrous Na₂SO₄, filtered, and concentrated to dryness to give 2-(tert-butyl) 3-ethyl (1 S,3S,5S)-5-(2-oxoethyl)-2-azabicyclo[3.1.0]hexane-2,3-dicarboxylate (393 mg, yield 84.3%) as a light-yellow oil, which was used directly in the next reaction without further purification. LCMS (ESI) m/z: 298 (M+H)⁺.

### Step 4: 2-((1S,3S,5S)-2-(tert-butoxycarbonyl)-3-(ethoxycarbonyl)-2-azabicyclo[3.1.0]hexan-5-yl)acetic acid

To a mixture of 2-(tert-butyl) 3-ethyl (1S,3S,5S)-5-(2-oxoethyl)-2-azabicyclo[3.1.0]hexane-2,3-dicarboxylate (393 mg, 1.32 mmol) and 2-methyl-2-butene (2.6 g, 36.96 mmol) in t-BuOH (5 mL) and H₂O (2 mL) was added NaH₂PO₄ (79 mg, 0.66 mmol) and NaClO₂ (300 mg, 3.30 mmol) and the reaction mixture was stirred at 25° C. for 2 hours. The mixture was diluted with EtOAc, washed with water and brine, dried over anhydrous Na₂SO₄, filtered, and concentrated to give 2-((1S,3S,5S)-2-(tert-butoxycarbonyl)-3-(ethoxycarbonyl)-2-azabicyclo[3.1.0]hexan-5-yl)acetic acid (400 mg, yield 96.6%) as a yellow oil, which was used directly in the next reaction without further purification. LC/MS (ESI) m/z: 314 (M+H)⁺.

### Step 5: 2-(Tert-butyl) 3-ethyl (1S,3S,5S)-5-(2-amino-2-oxoethyl)-2-azabicyclo[3.1.0]hexane-2,3-dicarboxylate

To a mixture of 2-((1S,3S,5S)-2-(tert-butoxycarbonyl)-3-(ethoxycarbonyl)-2-azabicyclo [3.1.0]hexan-5-yl)acetic acid (612 mg, 1.96 mmol) and NH₄Cl (155 mg, 2.93 mmol) in DMF (5 mL) was added DIPEA (1.51 g, 9.80 mmol) and HBTU (892 mg, 2.35 mmol) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-10% MeOH in DCM) to give 2-(tert-butyl) 3-ethyl (1S,3S,5S)-5-(2-amino-2-oxoethyl)-2-azabicyclo[3.1.0]hexane-2,3-dicarboxylate (580 mg, yield 95.1%) as a yellow oil. LC/MS (ESI) m/z: 313 (M+H)⁺.

### Step 6: (1S,3S,5S)-5-(2-amino-2-oxoethyl)-2-(tert-butoxycarbonyl)-2-azabicyclo [3.1.0]hexane-3-carboxylic acid

To a solution of 2-(tert-butyl) 3-ethyl (1S,3S,5S)-5-(2-amino-2-oxoethyl)-2-azabicyclo[3.1.0]hexane-2,3-dicarboxylate (250 mg, 0.80 mmol) in MeOH (3 mL) and H₂O (1 mL) was added LiOH·H₂O (67 mg, 1.60 mmol) and the mixture was stirred at room temperature for 2 hours. The mixture was acidified with 1 N aq·HCl to pH˜3 and extracted with EtOAc twice. The combined organic layers were washed with brine, dried over anhydrous Na₂SO₄, filtered, and concentrated under reduced pressure to dryness to give (1 S,3S,5S)-5-(2-amino-2-oxoethyl)-2-(tert-butoxycarbonyl)-2-azabicyclo[3.1.0]hexane-3-carboxylic acid (200 mg, yield 88.1%) as a yellow solid, which was used directly in the next step without further purification. LC/MS (ESI) m/z: 285 (M+H)⁺.

### Step 7: 3-Benzyl 2-(tert-butyl) (1S,3S,5S)-5-(2-amino-2-oxoethyl)-2-azabicyclo[3.1.0]hexane-2,3-dicarboxylate

To a mixture of (1S,3S,5S)-5-(2-amino-2-oxoethyl)-2-(tert-butoxycarbonyl)-2-azabicyclo[3.1.0]hexane-3-carboxylic acid (200 mg, 0.70 mmol) and K₂CO₃ (292 mg, 2.10 mmol) in DMF (3 mL) was added BnBr (144 mg, 0.84 mmol) at 0° C. and the mixture was stirred at room temperature for 2 hours. The mixture was diluted with EtOAc, washed with saturated aq·NH₄Cl solution and brine, dried over anhydrous Na₂SO₄, filtered, and concentrated under reduced pressure to dryness. The residue was purified by flash chromatography (silica gel, 0-5% MeOH in DCM) to give 3-benzyl 2-(tert-butyl) (1S,3S,5S)-5-(2-amino-2-oxoethyl)-2-azabicyclo[3.1.0]hexane-2,3-dicarboxylate (70 mg, yield 26.6%) as a yellow oil. LCMS (ESI) (m/z): 375 (M+H)⁺.

### Step 6: 3-Benzyl 2-(tert-butyl) (1S,3S,5S)-5-(2-(((E)-(dimethylamino)methylene) amino)-2-oxoethyl)-2-azabicyclo[3.1.0]hexane-2,3-dicarboxylate

A solution of 3-benzyl 2-(tert-butyl) (1 S,3S,5S)-5-(2-amino-2-oxoethyl)-2-azabicyclo [3.1.0]hexane-2,3-dicarboxylate (70 mg, 0.19 mmol) in DMF-DMA (3 mL) was stirred under N₂ atmosphere at 50° C. for 2 hours. The reaction mixture was concentrated under reduced pressure to dryness to give 3-benzyl 2-(tert-butyl) (1S,3S,5S)-5-(2-(((E)-(dimethylamino)methylene)amino)-2-oxoethyl)-2-azabicyclo[3.1.0]hexane-2,3-dicarboxylate (78 mg, yield 97.5%) as a brown solid, which was used directly in the next step without further purification. LCMS (ESI) m/z: 430 (M+H)⁺.

### Step 9: 3-Benzyl 2-(tert-butyl) (1S,3S,5S)-5-((1,2,4-oxadiazol-5-yl)methyl)-2-azabicyclo[3.1.0]hexane-2,3-dicarboxylate

To a mixture of 3-benzyl 2-(tert-butyl) (1S,3S,5S)-5-(2-(((E)-(dimethylamino)methylene) amino)-2-oxoethyl)-2-azabicyclo[3.1.0]hexane-2,3-dicarboxylate (80 mg, 0.19 mmol) and NH₂OH·HCl (19 mg, 0.28 mmol) in EtOH (3 mL) was added AcONa (31 mg, 0.38 mmol) and the mixture was stirred at 85° C. for 16 hours. The mixture was diluted with EtOAc, washed with water and brine, dried over anhydrous Na₂SO₄, filtered, and concentrated under reduced pressure to dryness. The residue was purified by flash chromatography (silica gel, 0-25% EtOAc in PE) to give 3-benzyl 2-(tert-butyl) (1S,3S,5S)-5-((1,2,4-oxadiazol-5-yl)methyl)-2-azabicyclo[3.1.0]hexane-2,3-dicarboxylate (32 mg, yield 43.2%) as a colorless oil. LCMS (ESI) (m/z): 400 (M+H)⁺.

### Step 10: Benzyl (1S,3S,5S)-5-((1,2,4-oxadiazol-5-yl)methyl)-2-azabicyclo[3.1.0]hexane-3-carboxylate hydrochloride

A solution of 3-benzyl 2-(tert-butyl) (1 S,3S,5S)-5-((1,2,4-oxadiazol-5-yl)methyl)-2-azabicyclo[3.1.0]hexane-2,3-dicarboxylate (30 mg, 0.08 mmol) in HCl/1,4-dioxane (2 mL, 4M) was stirred under N₂ atmosphere at room temperature for 2 hours. The reaction mixture was concentrated under reduced pressure to dryness to give benzyl (1S,3S,5S)-5-((1,2,4-oxadiazol-5-yl)methyl)-2-azabicyclo[3.1.0]hexane-3-carboxylate hydrochloride (23 mg, yield 90.9%) as a yellow solid, which was used directly in the next reaction without further purification. LCMS (ESI) m/z: 300 (M+H)⁺.

### Step 11: Benzyl (1S,3S,5S)-5-((1,2,4-oxadiazol-5-yl)methyl)-2-((9,9-difluoro-9H-fluorene-3-carbonyl)glycyl)-2-azabicyclo[3.1.0]hexane-3-carboxylate

To a mixture of (9,9-difluoro-9H-fluorene-3-carbonyl)glycine (20 mg, 0.07 mmol) and benzyl (1S,3S,5S)-5-((1,2,4-oxadiazol-5-yl)methyl)-2-azabicyclo[3.1.0]hexane-3-carboxylate hydrochloride (23 mg, 0.07 mmol) in DMF (3 mL) was added DIPEA (51 mg, 0.42 mmol) and T₃P (126 mg, 0.21 mmol, 50% wt. in EtOAc) at 0° C. under N₂ atmosphere and the mixture was stirred at room temperature for 1 hour. The mixture was quenched with saturated aq·NaHCO₃ solution and extracted with EtOAc twice. The combined organic layers were washed with brine, dried over anhydrous Na₂SO₄, filtered, and concentrated under reduced pressure to dryness. The residue was purified by prep-TLC (DCM:MeOH=10:1) to give benzyl (1S,3S,5S)-5-((1,2,4-oxadiazol-5-yl)methyl)-2-((9,9-difluoro-9H-fluorene-3-carbonyl)glycyl)-2-azabicyclo [3.1.0]hexane-3-carboxylate (23 mg, yield 60.5%) as a yellow solid. LC/MS (ESI) m/z: 585 (M+H)⁺.

### Step 12: (1S,3S,5S)-5-((1,2,4-oxadiazol-5-yl)methyl)-2-((9,9-difluoro-9H-fluorene-3-carbonyl)glycyl)-2-azabicyclo[3.1.0]hexane-3-carboxylic acid

To a solution of benzyl (1S,3S,5S)-5-((1,2,4-oxadiazol-5-yl)methyl)-2-((9,9-difluoro-9H-fluorene-3-carbonyl)glycyl)-2-azabicyclo[3.1.0]hexane-3-carboxylate (23 mg, 0.04 mmol) in MeOH (1 mL) and H₂O (0.3 mL) was added LiOH·H₂O (3.3 mg, 0.08 mmol) and the reaction mixture was stirred at room temperature for 2 hours. The mixture was acidified with 1 N aq·HCl to pH˜3 and extracted with EtOAc twice. The combined organic layers were dried over anhydrous Na₂SO₄, filtered, and concentrated under reduced pressure to dryness to give (1S,3S,5S)-5-((1,2,4-oxadiazol-5-yl)methyl)-2-((9,9-difluoro-9H-fluorene-3-carbonyl)glycyl)-2-azabicyclo[3.1.0]hexane-3-carboxylic acid (14 mg, yield 73.7%) as a yellow solid, which was used directly in the next step without further purification. LC/MS (ESI) m/z: 495 (M+H)⁺.

### Step 13: (1S,3S,5S)-5-((1,2,4-oxadiazol-5-yl)methyl)-N—((R)-1-(4-carbamimidoylthiophen-2-yl)ethyl)-2-((9,9-difluoro-9H-fluorene-3-carbonyl)glycyl)-2-azabicyclo[3.1.0]hexane-3-carboxamide (Compound 420)

To a mixture of (1 S,3S,5S)-5-((1,2,4-oxadiazol-5-yl)methyl)-2-((9,9-difluoro-9H-fluorene-3-carbonyl)glycyl)-2-azabicyclo[3.1.0]hexane-3-carboxylic acid (20 mg, 0.04 mmol) and (R)-5-(1-aminoethyl)thiophene-3-carboximidamide hydrochloride (14 mg, 0.08 mmol) in DMF (3 mL) was added DIPEA (31 mg, 0.24 mmol) and PyBop (25 mg, 0.05 mmol) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=8:1) and further purified by prep-HPLC to give Compound 420 (3 mg, yield 11.5%) as a white solid. ¹H NMR (400 MHz, CD₃OD) δ 8.49 (s, 1H), 8.18 (s, 2H), 7.87 (d, J=7.1 Hz, 1H), 7.76 (d, J=7.1 Hz, 1H), 7.71 (d, J=8.4 Hz, 1H), 7.65 (d, J=7.2 Hz, 1H), 7.58 (t, J=7.5 Hz, 1H), 7.46 (dd, J=18.0, 10.0 Hz, 2H), 5.34 (t, J=4.6 Hz, 1H), 4.58 (s, 1H), 4.40 (dd, J=28.2, 16.4 Hz, 2H), 3.59-3.54 (m, 1H), 2.41 (s, 2H), 2.19 (t, J=7.6 Hz, 2H), 1.58 (d, J=7.1 Hz, 3H), 1.24-1.19 (m, 1H), 0.95 (t, J=5.2 Hz, 1H). LC/MS (ESI) m/z: 646 (M+H)⁺.

### Scheme 395: Synthesis of (2S,4R)—N—((R)-1-(4-carbamimidoylthiophen-2-yl)ethyl)-4-(difluoromethoxy)-1-((9-methyl-9H-fluorene-3-carbonyl)glycyl)pyrrolidine-2-carboxamide (Compound 421)

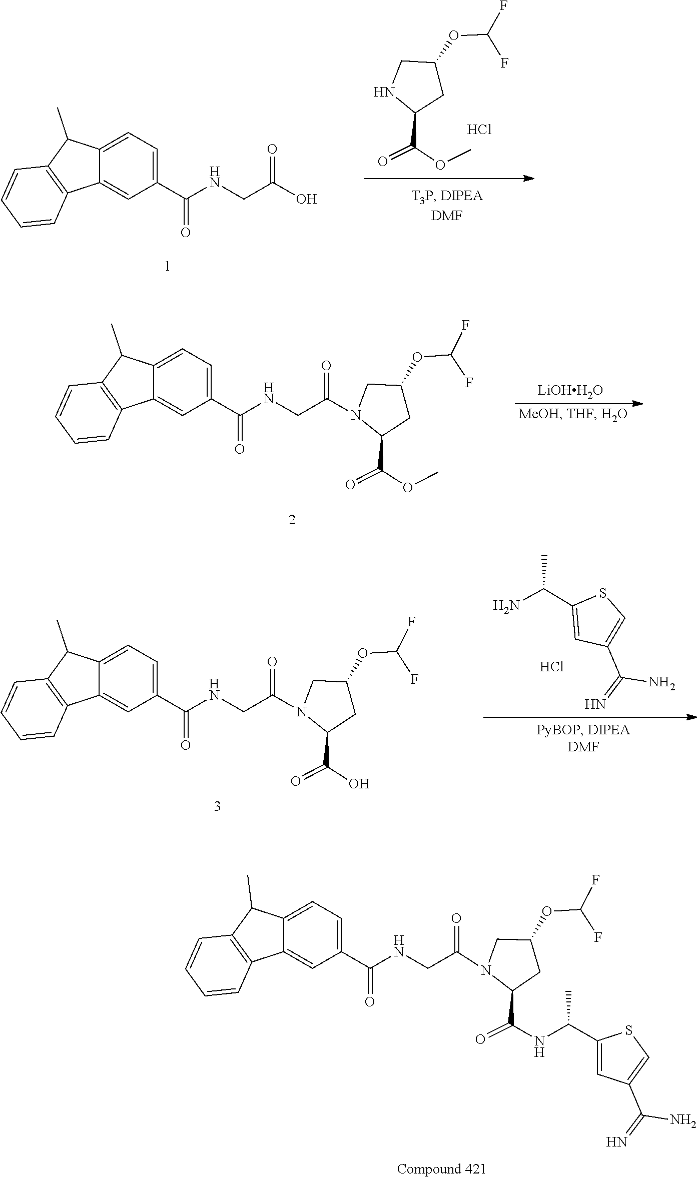

Compound 421 was prepared from (9-methyl-9H-fluorene-3-carbonyl)glycine, methyl (2S,4R)-4-(difluoromethoxy)pyrrolidine-2-carboxylate hydrochloride, and (R)-5-(1-aminoethyl) thiophene-3-carboximidamide hydrochloride based on the procedures set forth in Scheme 3. ¹H NMR (400 MHz, CD₃OD) δ 8.53 (s, 1H), 8.28 (d, J=9.2 Hz, 1H), 8.21 (s, 1H), 7.83 (d, J=7.8 Hz, 2H), 7.64 (d, J=7.7 Hz, 1H), 7.57 (d, J=6.5 Hz, 1H), 7.54 (s, 1H), 7.38 (t, J=6.1 Hz, 2H), 6.51 (dd, 1H), 5.31 (dd, 1H), 5.04 (s, 1H), 4.71-4.59 (m, 1H), 4.32 (d, J=16.6 Hz, 1H), 4.20 (d, J=16.6 Hz, 1H), 3.99 (dd, J=13.4, 6.1 Hz, 2H), 3.91 (d, J=10.4 Hz, 1H), 2.54-2.45 (m, 1H), 2.30-2.21 (m, 1H), 1.63 (dd, J=6.9 Hz, 3H), 1.53 (d, J=7.4 Hz, 3H). LC/MS (ESI) m/z: 596 (M+H)⁺.

### Scheme 396: Synthesis of (1S,3S,5R)—N—((R)-1-(4-carbamimidoylthiophen-2-yl)ethyl)-2-((9,9-difluoro-9H-fluorene-3-carbonyl)glycyl)-5-((3-(dimethylamino)propoxy) methyl)-2-azabicyclo[3.1.]hexane-3-carboxamide (Compound 422)

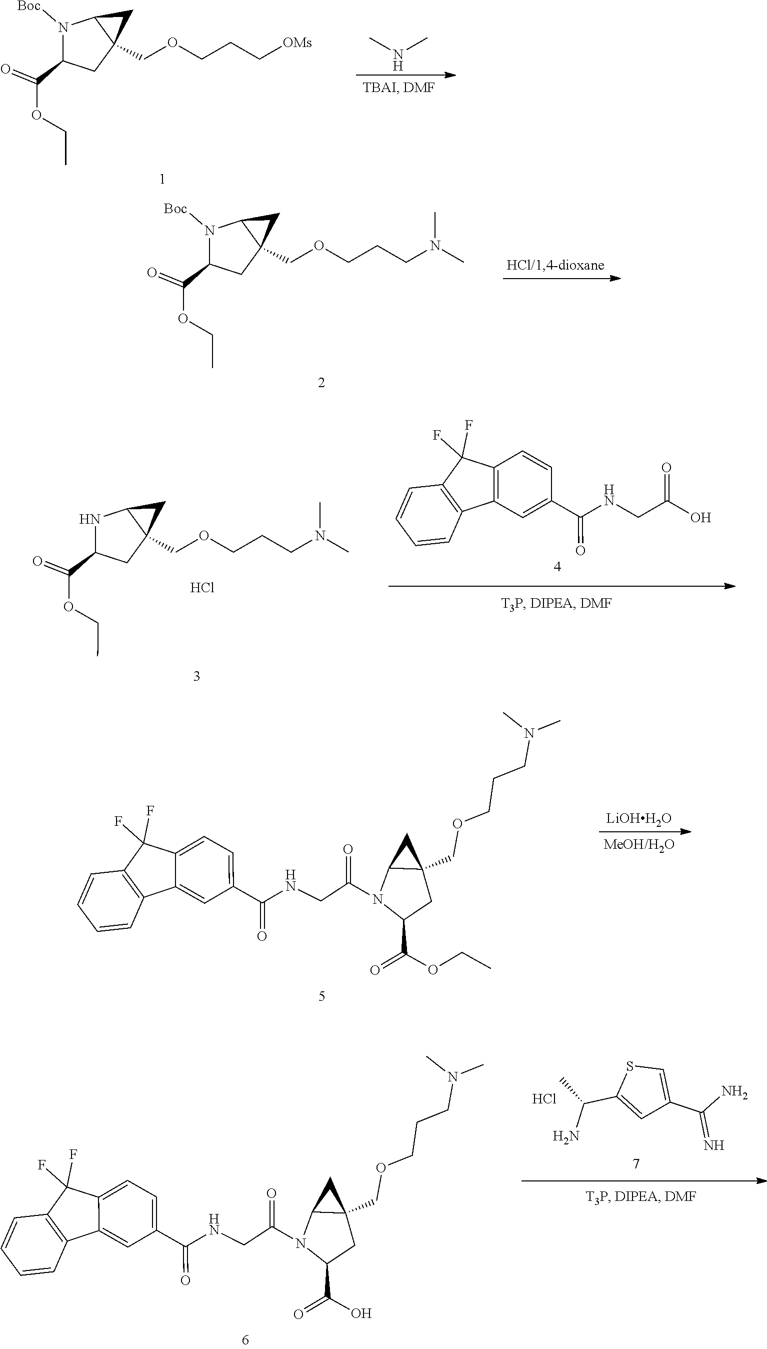

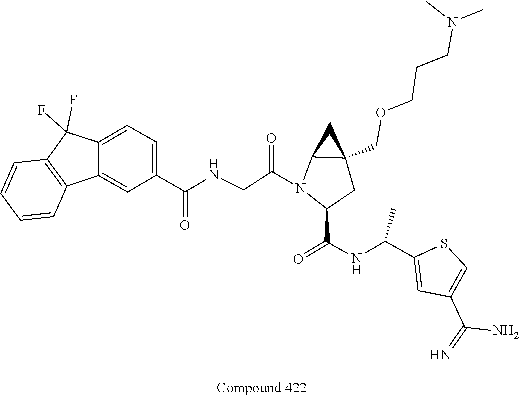 [SMILES: C[C@@H](NC(=O)[C@@H]1C[C@]2(COCCCN(C)C)C[C@@H]2N1C(=O)CNC(=O)c1ccc2c(c1)-c1ccccc1C2(F)F)c1cc(C(=N)N)cs1]

### Step 1: 2-(Tert-butyl) 3-ethyl (1S,3S,5R)-5-((3-(dimethylamino)propoxy)methyl)-2-azabicyclo[3.1.0]hexane-2,3-dicarboxylate (1)

To a mixture of 2-(tert-butyl) 3-ethyl (1S,3S,5R)-5-((3-((methylsulfonyl)oxy)propoxy) methyl)-2-azabicyclo[3.1.0]hexane-2,3-dicarboxylate (235 mg, 0.56 mmol) and dimethylamine (51 mg, 1.13 mmol) in DMF (2.5 mL) was added TBAI (5 mg, 0.014 mmol) at 25° C. under N₂ atmosphere and the reaction mixture was degassed under N₂ atmosphere for three times and stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by prep-TLC (DCM:MeOH=10:1) to give 2-(tert-butyl) 3-ethyl (1 S,3S,5R)-5-((3-(dimethylamino)propoxy)methyl)-2-azabicyclo [3.1.0]hexane-2,3-dicarboxylate (160 mg, yield 77.1%) as a colorless oil. LC/MS (ESI) m/z: 371 (M+H)⁺.

### Step 2: Ethyl (1S,3S,5R)-5-((3-(dimethylamino)propoxy)methyl)-2-azabicyclo[3.1.0]hexane-3-carboxylate hydrochloride (2)

A solution of 2-(tert-butyl) 3-ethyl (1S,3S,5R)-5-((3-(dimethylamino)propoxy)methyl)-2-azabicyclo[3.1.0]hexane-2,3-dicarboxylate (160 mg, 0.43 mmol) in HCl/1,4-dioxane (2 mL, 4M) was stirred under N₂ atmosphere at room temperature for 2 hours. The reaction mixture was concentrated under reduced pressure to dryness to give ethyl (1S,3S,5R)-5-((3-(dimethylamino)propoxy)methyl)-2-azabicyclo[3.1.0]hexane-3-carboxylate hydrochloride (130 mg, yield 98.6%) as a yellow solid, which was used directly in the next step without further purification. LCMS (ESI) m/z: 271 (M+H)⁺.

### Step 3: Ethyl (1S,3S,5R)-2-((9,9-difluoro-9H-fluorene-3-carbonyl)glycyl)-5-((3-(dimethylamino)propoxy)methyl)-2-azabicyclo[3.1.0]hexane-3-carboxylate (3)

To a mixture of (9,9-difluoro-9H-fluorene-3-carbonyl)glycine (95 mg, 0.31 mmol) and ethyl (1S,3S,5R)-5-((3-(dimethylamino)propoxy)methyl)-2-azabicyclo[3.1.0]hexane-3-carboxylate hydrochloride (133 mg, 0.43 mmol) in DMF (1 mL) was added DIPEA (129 mg, 1.0 mmol) and T₃P (286 mg, 0.45 mmol, 50% wt. in EtOAc) at 0° C. under N₂ atmosphere and the mixture was stirred at room temperature for 1 hour. The mixture was quenched with saturated aq·NaHCO₃ solution and extracted with CHCl₃/i-PrOH (3/1, v/v) twice. The combined organic layers were washed with brine, dried over anhydrous Na₂SO₄, filtered, and concentrated under reduced pressure to dryness. The residue was purified by prep-TLC (DCM:MeOH=10:1) to give ethyl (1S,3S,5R)-2-((9,9-difluoro-9H-fluorene-3-carbonyl)glycyl)-5-((3-(dimethylamino)propoxy)methyl)-2-azabicyclo[3.1.0]hexane-3-carboxylate (110 mg, yield 63.9%) as a white solid. LCMS (ESI) (m/z): 556 (M+H)⁺.

### Step 4: (1S,3S,5R)-2-((9,9-difluoro-9H-fluorene-3-carbonyl)glycyl)-5-((3-(dimethylamino)propoxy)methyl)-2-azabicyclo[3.1.0]hexane-3-carboxylic acid (4)

To a solution of ethyl (1S,3S,5R)-2-((9,9-difluoro-9H-fluorene-3-carbonyl)glycyl)-5-((3-(dimethylamino)propoxy)methyl)-2-azabicyclo[3.1.0]hexane-3-carboxylate (90 mg, 0.16 mmol) in MeOH/H₂O (3 mL, 2/1) was added LiOH·H₂O (24 mg, 0.60 mmol) and the reaction mixture was stirred at room temperature for 4 hours. The mixture was acidified with 1 N aq·HCl to pH˜3 and extracted with EtOAc twice. The combined organic layers were dried over anhydrous Na₂SO₄, filtered, and concentrated under reduced pressure to give (1S,3S,5R)-2-((9,9-difluoro-9H-fluorene-3-carbonyl)glycyl)-5-((3-(dimethylamino)propoxy)methyl)-2-azabicyclo[3.1.0]hexane-3-carboxylic acid (70 mg, yield 82.4%) as a white solid, which was used directly in the next step without further purification. LC/MS (ESI) m/z: 528 (M+H)⁺.

### Step 5: (1S,3S,5R)—N—((R)-1-(4-carbamimidoylthiophen-2-yl)ethyl)-2-((9,9-difluoro-9H-fluorene-3-carbonyl)glycyl)-5-((3-(dimethylamino)propoxy)methyl)-2-azabicyclo[3.1.0]hexane-3-carboxamide (Compound 422)

To a mixture of (1S,3S,5R)-2-((9,9-difluoro-9H-fluorene-3-carbonyl)glycyl)-5-((3-(dimethylamino)propoxy)methyl)-2-azabicyclo[3.1.0]hexane-3-carboxylic acid (35 mg, 0.066 mmol) and (R)-5-(1-aminoethyl)thiophene-3-carboximidamide hydrochloride (28 mg, 0.13 mmol) in DMF (0.5 mL) was added DIPEA (43 mg, 0.33 mmol) and PyBop (34 mg, 0.066 mmol) under N₂ atmosphere and the reaction mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=5:1) and further purified by prep-HPLC to give Compound 422 (3.7 mg, yield 8.3%) as a white solid. ¹H NMR (400 MHz, CD₃OD) δ 8.61 (s, 1H), 8.22 (d, J=1.5 Hz, 1H), 8.19 (s, 1H), 7.89 (d, J=7.8 Hz, 1H), 7.75 (dd, J=12.0, 7.8 Hz, 2H), 7.67 (d, J=7.4 Hz, 1H), 7.59 (t, J=7.6 Hz, 1H), 7.51 (s, 1H), 7.46 (t, J=7.6 Hz, 1H), 5.24 (q, J=7.0 Hz, 1H), 4.52 (d, J=16.5 Hz, 1H), 4.29 (d, J=16.5 Hz, 1H), 3.64-3.56 (m, 4H), 3.51 (d, J=10.2 Hz, 1H), 3.19-3.06 (m, 3H), 2.82 (s, 6H), 2.69 (t, J=12.2 Hz, 1H), 2.13 (dd, J=13.5, 3.7 Hz, 1H), 2.02-1.96 (m, 2H), 1.59 (d, J=7.0 Hz, 3H), 1.41-1.37 (m, 1H), 1.03 (t, J=5.3 Hz, 1H). LC/MS (ESI) m/z: 679 (M+H)⁺.

### Example 2. Non-Limiting Examples of Compounds of the Present Disclosure

Table 1 shows illustrative complement pathway with characterizing data. The assay of Example 3 was used to determine the IC₅₀'s of the compounds. Other standard complement assays are also available. Three ***s are used to denote compounds with an IC₅₀ less than 100 nanomolar; two **s indicates a compound with an IC₅₀ greater than 100 nanomolar and less than 1 micromolar, and one * denotes compounds with an IC₅₀ greater than 1 micromolar.

TABLE 1
Non-limiting Examples of Compounds of the Present Disclosure

| Cmp | | | IC₅₀ | RT min | MS |
|---|---|---|---|---|---|
| No. | Structure | Name | (Stars) | (Method A) | (M + 1) |
| 1 | 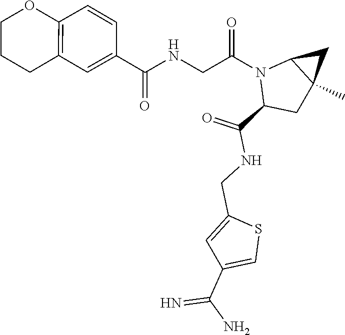 [SMILES: C[C@@]12C[C@@H]1N(C(=O)CNC(=O)c1ccc3c(c1)CCCO3)[C@H](C(=O)NCc1cc(C(=N)N)cs1)C2] | (1S,3S,5S)-N-((4- carbamimidoylthiophen- 2-yl)methyl)-2- ((chroman-6- carbonyl)glycyl)-5- methyl-2- azabicyclo[3.1.0]hexane- 3-carboxamide | *** | 1.08 | 496 |
| 2 | 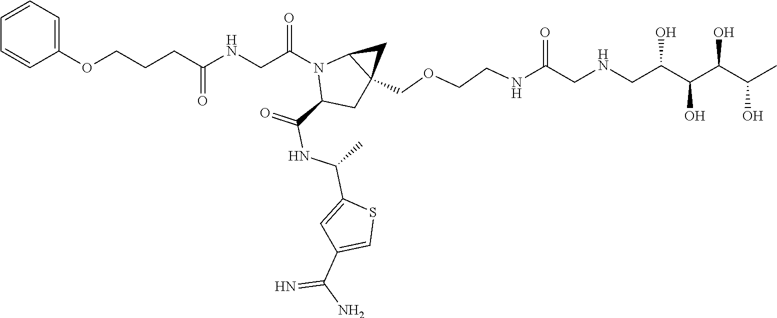 [SMILES: C[C@H](O)[C@H](O)[C@@H](O)[C@@H](O)CNCC(=O)NCCOC[C@@]12C[C@@H]1N(C(=O)CNC(=O)CCCOc1ccccc1)[C@H](C(=O)N[C@H](C)c1cc(C(=N)N)cs1)C2] | (1S,3S,5R)-N-((R)-1-(4- carbamimidoylthiophen- 2-yl)ethyl)-2-((4- phenoxybutanoyl)glycyl)- 5-((2-(2- (((2S,3S,4S,5S)- 2,3,4,5- tetrahydroxyhexyl)amino) acetamido)ethoxy) methyl)-2- azabicyclo[3.1.0]hexane- 3-carboxamide | *** | 0.57 | 776 |
| 3 | 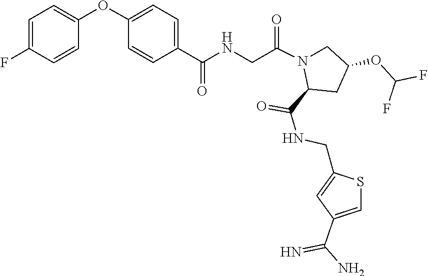 [SMILES: N=C(N)c1csc(CNC(=O)[C@@H]2C[C@@H](OC(F)F)CN2C(=O)CNC(=O)c2ccc(Oc3ccc(F)cc3)cc2)c1] | (2S,4R)-N-((4- carbamimidoylthiophen- 2-yl)methyl)-4- (difluoromethoxy)-1-((4- (4- fluorophenoxy)benzoyl) glycyl)pyrrolidine-2- carboxamide | *** | 1.46 | 590 |
| 4 | 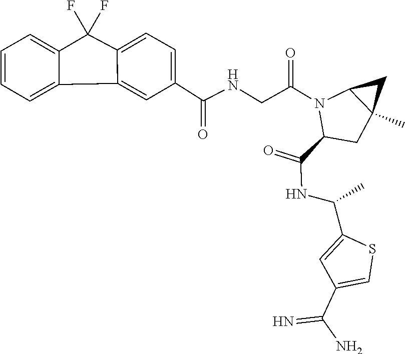 [SMILES: C[C@@H](NC(=O)[C@@H]1C[C@]2(C)C[C@@H]2N1C(=O)CNC(=O)c1ccc2c(c1)-c1ccccc1C2(F)F)c1cc(C(=N)N)cs1] | (1S,3S,5S)-N-((R)-1-(4- carbamimidoylthiophen- 2-yl)ethyl)-2-((9,9- difluoro-9H-fluorene-3- carbonyl)glycyl)-5- methyl-2- azabicyclo[3.1.0]hexane- 3-carboxamide | *** | 1.63 | 578 |
| 5 | 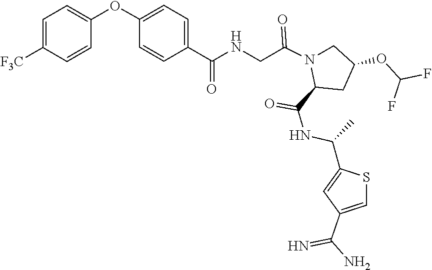 [SMILES: C[C@@H](NC(=O)[C@@H]1C[C@@H](OC(F)F)CN1C(=O)CNC(=O)c1ccc(Oc2ccc(C(F)(F)F)cc2)cc1)c1cc(C(=N)N)cs1] | (2S,4R)-N-((R)-1-(4- carbamimidoylthiophen- 2-yl)ethyl)-4- (difluoromethoxy)-1-((4- (4- (trifluoromethyl)phenoxy) benzoyl)glycyl) pyrrolidine-2- carboxamide | *** | 1.78 | 654 |
| 6 | 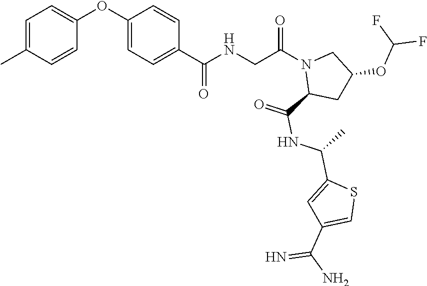 [SMILES: Cc1ccc(Oc2ccc(C(=O)NCC(=O)N3C[C@H](OC(F)F)C[C@H]3C(=O)N[C@H](C)c3cc(C(=N)N)cs3)cc2)cc1] | (2S,4R)-N-((R)-1-(4- carbamimidoylthiophen- 2-yl)ethyl)-4- (difluoromethoxy)-1-((4- (p- tolyloxy)benzoyl)glycyl) pyrrolidine-2- carboxamide | *** | 1.66 | 600 |
| 7 | 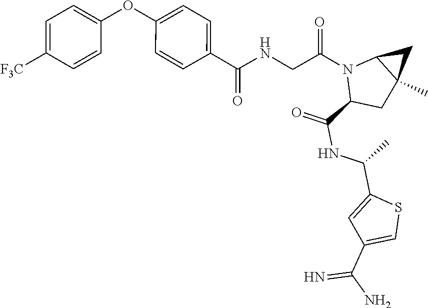 [SMILES: C[C@@H](NC(=O)[C@@H]1C[C@]2(C)C[C@@H]2N1C(=O)CNC(=O)c1ccc(Oc2ccc(C(F)(F)F)cc2)cc1)c1cc(C(=N)N)cs1] | (1S,3S,5S)-N-((R)-1-(4- carbamimidoylthiophen- 2-yl)ethyl)-5-methyl-2- ((4-(p- tolyloxy)benzoyl)glycyl)- 2- azabicyclo[3.1.0]hexane- 3-carboxamide | *** | 1.61 | 560 |
| 8 | 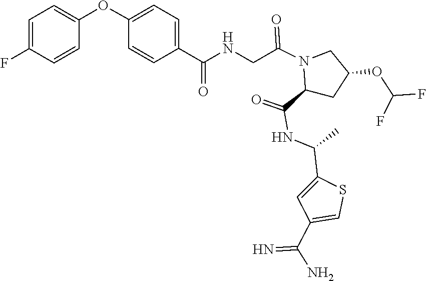 [SMILES: C[C@@H](NC(=O)[C@@H]1C[C@@H](OC(F)F)CN1C(=O)CNC(=O)c1ccc(Oc2ccc(F)cc2)cc1)c1cc(C(=N)N)cs1] | (2S,4R)-N-((R)-1-(4- carbamimidoylthiophen- 2-yl)ethyl)-4- (difluoromethoxy)-1-((4- (4- fluorophenoxy)benzoyl) glycyl)pyrrolidine-2- carboxamide | *** | 1.51 | 604 |
| 9 | 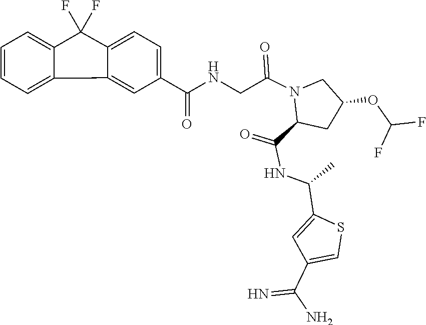 [SMILES: C[C@@H](NC(=O)[C@@H]1C[C@@H](OC(F)F)CN1C(=O)CNC(=O)c1ccc2c(c1)-c1ccccc1C2(F)F)c1cc(C(=N)N)cs1] | (2S,4R)-N-((R)-1-(4- carbamimidoylthiophen- 2-yl)ethyl)-1-((9,9- difluoro-9H-fluorene-3- carbonyl)glycyl)-4- (difluoromethoxy) pyrrolidine-2- carboxamide | *** | 1.59 | 618 |
| 10 | 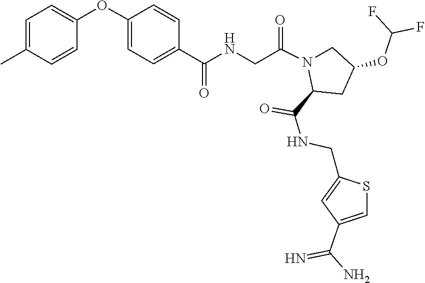 [SMILES: Cc1ccc(Oc2ccc(C(=O)NCC(=O)N3C[C@H](OC(F)F)C[C@H]3C(=O)NCc3cc(C(=N)N)cs3)cc2)cc1] | (2S,4R)-N-((4- carbamimidoylthiophen- 2-yl)methyl)-4- (difluoromethoxy)-1-((4- (p- tolyloxy)benzoyl)glycyl) pyrrolidine-2- carboxamide | *** | 1.61 | 586 |
| 11 | 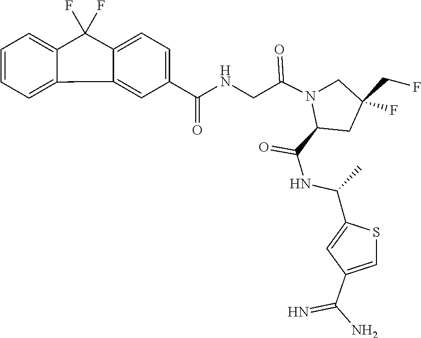 [SMILES: C[C@@H](NC(=O)[C@@H]1C[C@](F)(CF)CN1C(=O)CNC(=O)c1ccc2c(c1)-c1ccccc1C2(F)F)c1cc(C(=N)N)cs1] | (2S,4R)-N-((R)-1-(4- carbamimidoylthiophen- 2-yl)ethyl)-1-((9,9- difluoro-9H-fluorene-3- carbonyl)glycyl)-4- fluoro-4- (fluoromethyl)pyrrolidine- 2-carboxamide | *** | 1.54 | 602 |
| 12 | 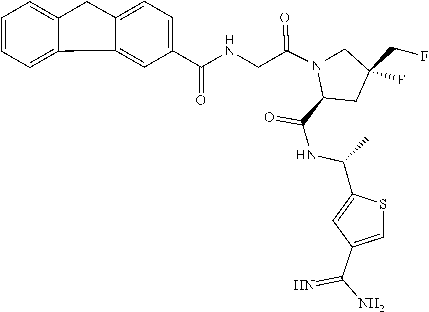 [SMILES: C[C@@H](NC(=O)[C@@H]1C[C@](F)(CF)CN1C(=O)CNC(=O)c1ccc2c(c1)-c1ccccc1C2)c1cc(C(=N)N)cs1] | (2S,4R)-1-((9H- fluorene-3- carbonyl)glycyl)-N-((R)- 1-(4- carbamimidoylthiophen- 2-yl)ethyl)-4-fluoro-4- (fluoromethyl)pyrrolidine- 2-carboxamide | *** | 1.42 | 566 |
| 13 | 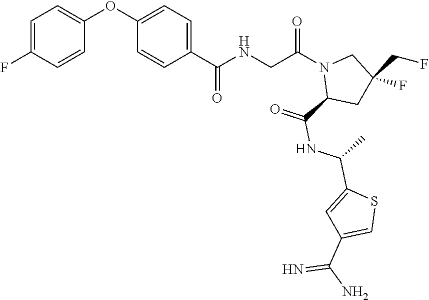 [SMILES: C[C@@H](NC(=O)[C@@H]1C[C@](F)(CF)CN1C(=O)CNC(=O)c1ccc(Oc2ccc(F)cc2)cc1)c1cc(C(=N)N)cs1] | (2S,4R)-N-((R)-1-(4- carbamimidoylthiophen- 2-yl)ethyl)-4-fluoro-4- (fluoromethyl)-1-((4-(4- fluorophenoxy)benzoyl) glycyl)pyrrolidine-2- carboxamide | *** | 1.46 | 588 |
| 14 | 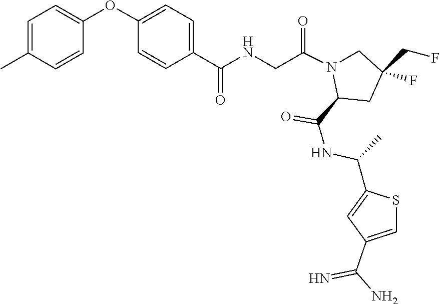 [SMILES: Cc1ccc(Oc2ccc(C(=O)NCC(=O)N3C[C@@](F)(CF)C[C@H]3C(=O)N[C@H](C)c3cc(C(=N)N)cs3)cc2)cc1] | (2S,4R)-N-((R)-1-(4- carbamimidoylthiophen- 2-yl)ethyl)-4-fluoro-4- (fluoromethyl)-1-((4-(p- tolyloxy)benzoyl)glycyl) pyrrolidine-2- carboxamide | *** | 1.57 | 584 |
| 15 | 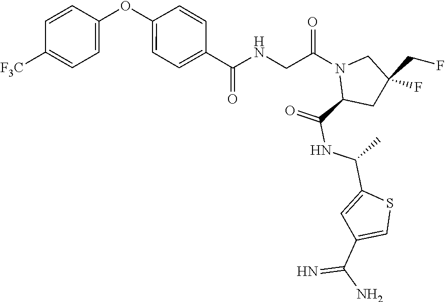 [SMILES: C[C@@H](NC(=O)[C@@H]1C[C@](F)(CF)CN1C(=O)CNC(=O)c1ccc(Oc2ccc(C(F)(F)F)cc2)cc1)c1cc(C(=N)N)cs1] | (2S,4R)-N-((R)-1-(4- carbamimidoylthiophen- 2-yl)ethyl)-4-fluoro-4- (fluoromethyl)-1-((4-(4- (trifluoromethyl)phenoxy) benzoyl)glycyl) pyrrolidine-2- carboxamide | *** | 1.73 | 638 |
| 16 | 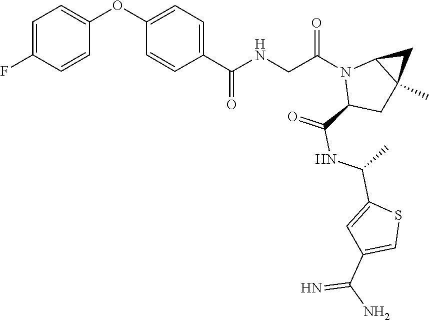 [SMILES: C[C@@H](NC(=O)[C@@H]1C[C@]2(C)C[C@@H]2N1C(=O)CNC(=O)c1ccc(Oc2ccc(F)cc2)cc1)c1cc(C(=N)N)cs1] | (1S,3S,5S)-N-((R)-1-(4- carbamimidoylthiophen- 2-yl)ethyl)-2-((4-(4- fluorophenoxy)benzoyl) glycyl)-5-methyl-2- azabicyclo[3.1.0]hexane- 3-carboxamide | *** | 1.52 | 564 |
| 17 | 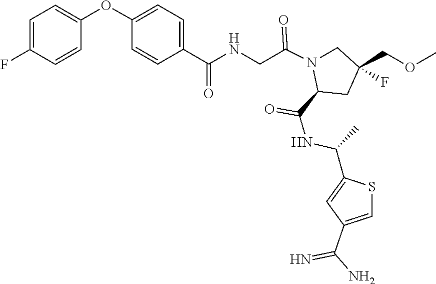 [SMILES: COC[C@@]1(F)C[C@@H](C(=O)N[C@H](C)c2cc(C(=N)N)cs2)N(C(=O)CNC(=O)c2ccc(Oc3ccc(F)cc3)cc2)C1] | (2S,4R)-N-((R)-1-(4- carbamimidoylthiophen- 2-yl)ethyl)-4-fluoro-1- ((4-(4- fluorophenoxy)benzoyl) glycyl)-4- (methoxymethyl) pyrrolidine-2- carboxamide | *** | 1.44 | 600 |
| 18 | 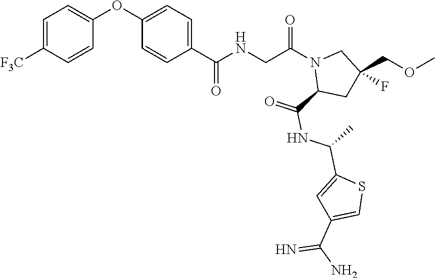 [SMILES: COC[C@@]1(F)C[C@@H](C(=O)N[C@H](C)c2cc(C(=N)N)cs2)N(C(=O)CNC(=O)c2ccc(Oc3ccc(C(F)(F)F)cc3)cc2)C1] | (2S,4R)-N-((R)-1-(4- carbamimidoylthiophen- 2-yl)ethyl)-4-fluoro-4- (methoxymethyl)-1-((4- (4- (trifluoromethyl)phenoxy) benzoyl)glycyl) pyrrolidine-2- carboxamide | *** | 1.71 | 650 |
| 19 | 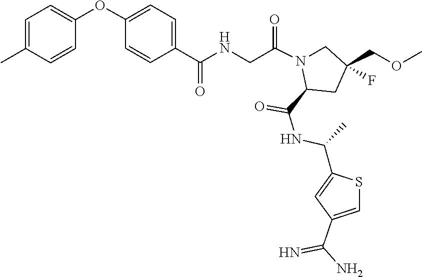 | (2S,4R)-N-((R)-1-(4- carbamimidoylthiophen- 2-yl)ethyl)-4-fluoro-4- (methoxymethyl)-1-((4- (p- tolyloxy)benzoyl)glycyl) pyrrolidine-2- carboxamide | *** | 1.54 | 596 |
| 20 | 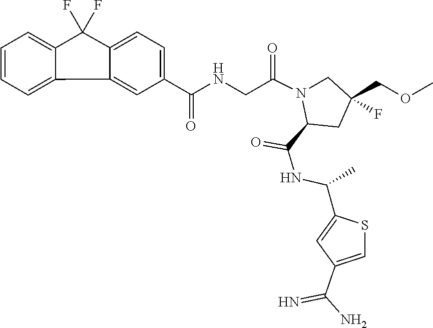 [SMILES: COC[C@@]1(F)C[C@@H](C(=O)N[C@H](C)c2cc(C(=N)N)cs2)N(C(=O)CNC(=O)c2ccc3c(c2)-c2ccccc2C3(F)F)C1] | (2S,4R)-N-((R)-1-(4- carbamimidoylthiophen- 2-yl)ethyl)-1-((9,9- difluoro-9H-fluorene-3- carbonyl)glycyl)-4- fluoro-4- (methoxymethyl) pyrrolidine-2- carboxamide | *** | 1.51 | 614 |
| 21 | 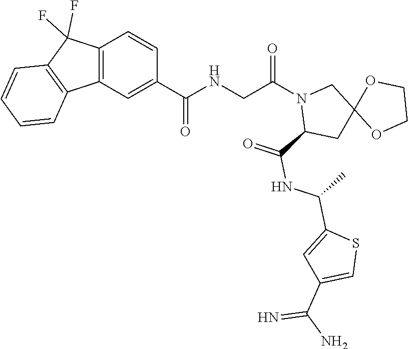 [SMILES: C[C@@H](NC(=O)[C@@H]1CC2(CN1C(=O)CNC(=O)c1ccc3c(c1)-c1ccccc1C3(F)F)OCCO2)c1cc(C(=N)N)cs1] | (S)-N-(R)-1-(4- carbamimidoylthiophen- 2-yl)ethyl)-7-((9,9- difluoro-9H-fluorene-3- carbonyl)glycyl)-1,4- dioxa-7- azaspiro[4.4]nonane-8- carboxamide | *** | 1.42 | 610 |
| 22 | 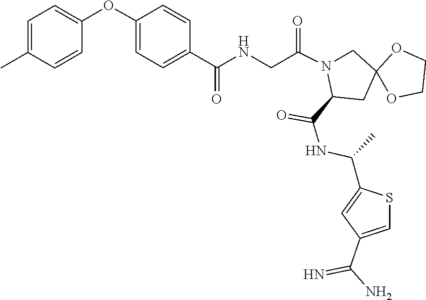 [SMILES: Cc1ccc(Oc2ccc(C(=O)NCC(=O)N3CC4(C[C@H]3C(=O)N[C@H](C)c3cc(C(=N)N)cs3)OCCO4)cc2)cc1] | (S)-N-((R)-1-(4- carbamimidoylthiophen- 2-yl)ethyl)-7-((4-(p- tolyloxy)benzoyl)glycyl)- 1,4-dioxa-7- azaspiro[4.4]nonane-8- carboxamide | *** | 1.50 | 592 |
| 23 | 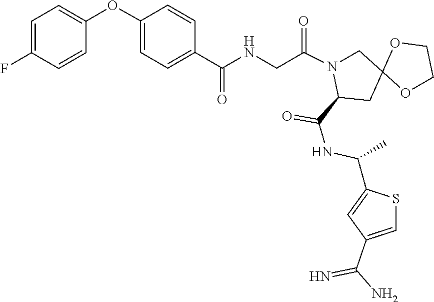 [SMILES: C[C@@H](NC(=O)[C@@H]1CC2(CN1C(=O)CNC(=O)c1ccc(Oc3ccc(F)cc3)cc1)OCCO2)c1cc(C(=N)N)cs1] | (S)-N-((R)-1-(4- carbamimidoylthiophen- 2-yl)ethyl)-7-((4-(4- fluorophenoxy)benzoyl) glycyl)-1,4-dioxa-7- azaspiro[4.4]nonane-8- carboxamide | *** | 1.43 | 596 |
| 24 | 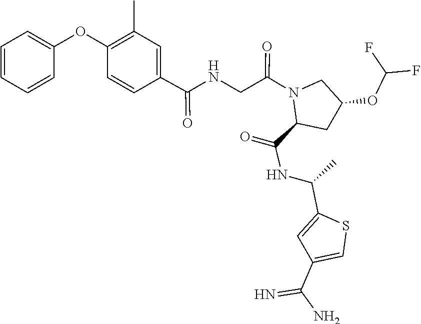 [SMILES: Cc1cc(C(=O)NCC(=O)N2C[C@H](OC(F)F)C[C@H]2C(=O)N[C@H](C)c2cc(C(=N)N)cs2)ccc1Oc1ccccc1] | (2S,4R)-N-((R)-1-(4- carbamimidoylthiophen- 2-yl)ethyl)-4- (difluoromethoxy)-1-((3- methyl-4- phenoxybenzoyl)glycyl) pyrrolidine-2- carboxamide | *** | 1.61 | 600 |
| 25 | 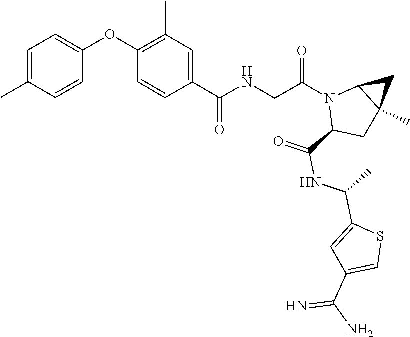 | (1S,3S,5S)-N-((R)-1-(4- carbamimidoylthiophen- 2-yl)ethyl)-5-methyl-2- ((3-methyl-4-(p- tolyloxy)benzoyl)glycyl)- 2- azabicyclo[3.1.0]hexane- 3-carboxamide | *** | 1.72 | 574 |
| 26 | 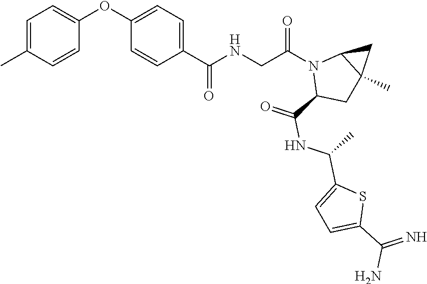 [SMILES: Cc1ccc(Oc2ccc(C(=O)NCC(=O)N3[C@H]4C[C@@]4(C)C[C@H]3C(=O)N[C@H](C)c3ccc(C(=N)N)s3)cc2)cc1] | (1S,3S,5S)-N-((R)-1-(5- carbamimidoylthiophen- 2-yl)ethyl)-5-methyl-2- ((4-(p- tolyloxy)benzoyl)glycyl)- 2- azabicyclo[3.1.0]hexane- 3-carboxamide | *** | 1.58 | 560 |
| 27 | 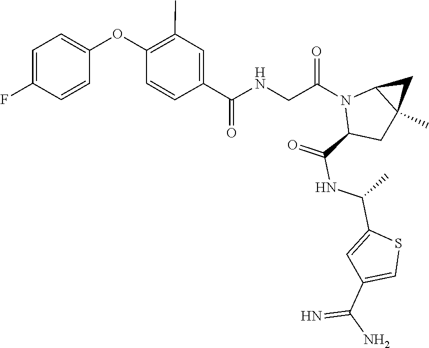 [SMILES: Cc1cc(C(=O)NCC(=O)N2[C@H]3C[C@@]3(C)C[C@H]2C(=O)N[C@H](C)c2cc(C(=N)N)cs2)ccc1Oc1ccc(F)cc1] | (1S,3S,5S)-N-((R)-1-(4- carbamimidoylthiophen- 2-yl)ethyl)-2-((4-(4- fluorophenoxy)-3- methylbenzoyl)glycyl)- 5-methyl-2- azabicyclo[3.1.0]hexane- 3-carboxamide | *** | 1.61 | 578 |
| 28 | 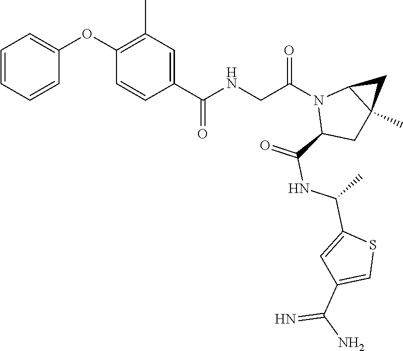 [SMILES: Cc1cc(C(=O)NCC(=O)N2[C@H]3C[C@@]3(C)C[C@H]2C(=O)N[C@H](C)c2cc(C(=N)N)cs2)ccc1Oc1ccccc1] | (1S,3S,5S)-N-((R)-1-(4- carbamimidoylthiophen- 2-yl)ethyl)-5-methyl-2- ((3-methyl-4- phenoxybenzoyl)glycyl)- 2- azabicyclo[3.1.0]hexane- 3-carboxamide | *** | 1.58 | 560 |
| 29 | 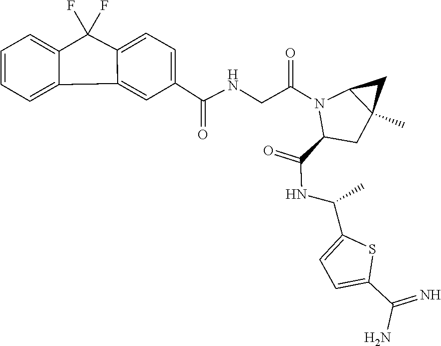 [SMILES: C[C@@H](NC(=O)[C@@H]1C[C@]2(C)C[C@@H]2N1C(=O)CNC(=O)c1ccc2c(c1)-c1ccccc1C2(F)F)c1ccc(C(=N)N)s1] | (1S,3S,5S)-N-((R)-1-(5- carbamimidoylthiophen- 2-yl)ethyl)-2-((9,9- difluoro-9H-fluorene-3- carbonyl)glycyl)-5- methyl-2- azabicyclo[3.1.0]hexane- 3-carboxamide | *** | 1.58 | 578 |
| 30 | 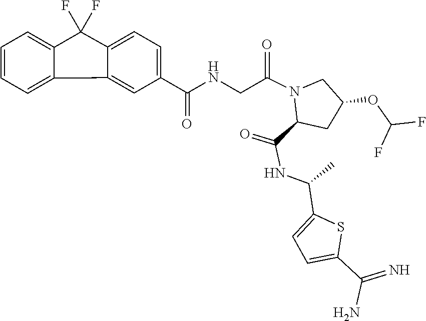 [SMILES: C[C@@H](NC(=O)[C@@H]1C[C@@H](OC(F)F)CN1C(=O)CNC(=O)c1ccc2c(c1)-c1ccccc1C2(F)F)c1ccc(C(=N)N)s1] | (2S,4R)-N-((R)-1-(5- carbamimidoylthiophen- 2-yl)ethyl)-1-((9,9- difluoro-9H-fluorene-3- carbonyl)glycyl)-4- (difluoromethoxy) pyrrolidine-2- carboxamide | *** | 1.56 | 618 |
| 31 | 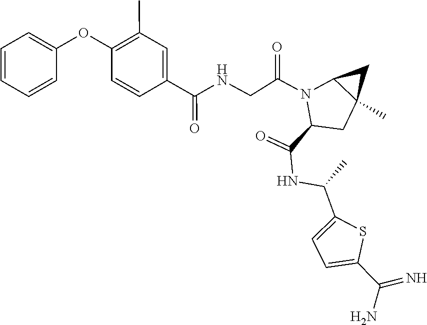 [SMILES: Cc1cc(C(=O)NCC(=O)N2[C@H]3C[C@@]3(C)C[C@H]2C(=O)N[C@H](C)c2ccc(C(=N)N)s2)ccc1Oc1ccccc1] | (1S,3S,5S)-N-((R)-1-(5- carbamimidoylthiophen- 2-yl)ethyl)-5-methyl-2- ((3-methyl-4- phenoxybenzoyl)glycyl)- 2- azabicyclo[3.1.0]hexane- 3-carboxamide | *** | 1.56 | 560 |
| 32 | 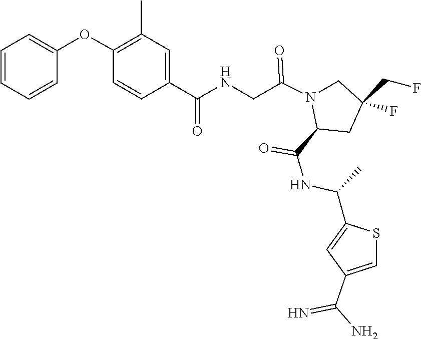 [SMILES: Cc1cc(C(=O)NCC(=O)N2C[C@@](F)(CF)C[C@H]2C(=O)N[C@H](C)c2cc(C(=N)N)cs2)ccc1Oc1ccccc1] | (2S,4R)-N-((R)-1-(4- carbamimidoylthiophen- 2-yl)ethyl)-4-fluoro-4- (fluoromethyl)-1-((3- methyl-4- phenoxybenzoyl)glycyl) pyrrolidine-2- carboxamide | *** | 1.54 | 584 |
| 33 | 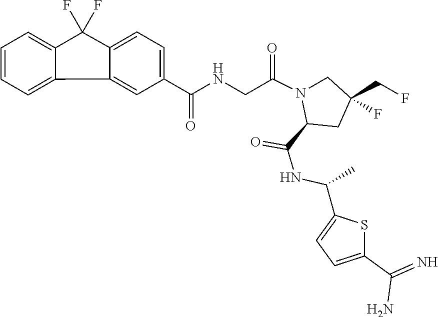 [SMILES: C[C@@H](NC(=O)[C@@H]1C[C@](F)(CF)CN1C(=O)CNC(=O)c1ccc2c(c1)-c1ccccc1C2(F)F)c1ccc(C(=N)N)s1] | (2S,4R)-N-((R)-1-(5- carbamimidoylthiophen- 2-yl)ethyl)-1-((9,9- difluoro-9H-fluorene-3- carbonyl)glycyl)-4- fluoro-4- (fluoromethyl)pyrrolidine- 2-carboxamide | *** | 1.54 | 602 |
| 34 | 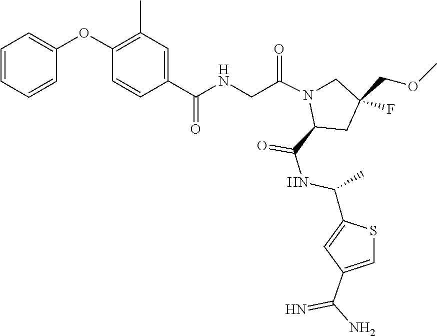 [SMILES: COC[C@@]1(F)C[C@@H](C(=O)N[C@H](C)c2cc(C(=N)N)cs2)N(C(=O)CNC(=O)c2ccc(Oc3ccccc3)c(C)c2)C1] | (2S,4R)-N-((R)-1-(4- carbamimidoylthiophen- 2-yl)ethyl)-4-fluoro-4- (methoxymethyl)-1-((3- methyl-4- phenoxybenzoyl)glycyl) pyrrolidine-2- carboxamide | *** | 1.50 | 596 |
| 35 | 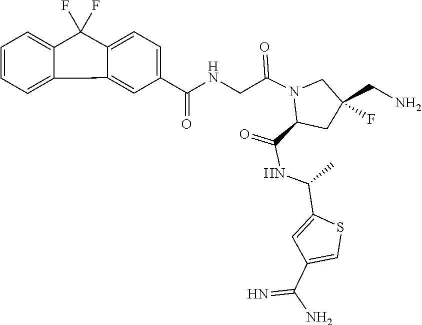 [SMILES: C[C@@H](NC(=O)[C@@H]1C[C@](F)(CN)CN1C(=O)CNC(=O)c1ccc2c(c1)-c1ccccc1C2(F)F)c1cc(C(=N)N)cs1] | (2S,4S)-4- (aminomethyl)-N-((R)- 1-(4- carbamimidoylthiophen- 2-yl)ethyl)-1-((9,9- difluoro-9H-fluorene-3- carbonyl)glycyl)-4- fluoropyrrolidine-2- carboxamide | *** | 0.96 | 599 |
| 36 | 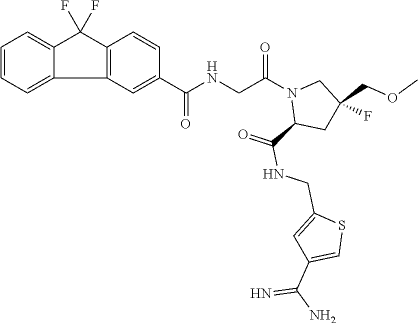 [SMILES: COC[C@@]1(F)C[C@@H](C(=O)NCc2cc(C(=N)N)cs2)N(C(=O)CNC(=O)c2ccc3c(c2)-c2ccccc2C3(F)F)C1] | (2S,4S)-N-((R)-1-(4- carbamimidoylthiophen- 2-yl)ethyl)-1-((9,9- difluoro-9H-fluorene-3- carbonyl)glycyl)-4- ((dimethylamino)methyl)- 4-fluoropyrrolidine-2- carboxamide | *** | 1.05 | 627 |
| 37 | 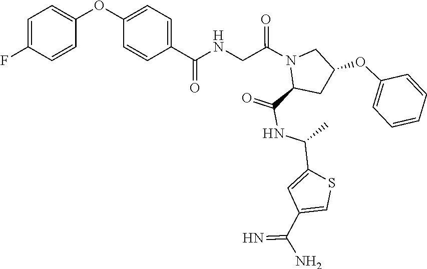 [SMILES: C[C@@H](NC(=O)[C@@H]1C[C@@H](Oc2ccccc2)CN1C(=O)CNC(=O)c1ccc(Oc2ccc(F)cc2)cc1)c1cc(C(=N)N)cs1] | (2S,4R)-N-((R)-1-(4- carbamimidoylthiophen- 2-yl)ethyl)-1-((4-(4- fluorophenoxy)benzoyl) glycyl)-4- phenoxypyrrolidine-2- carboxamide | *** | 1.74 | 630 |
| 38 | 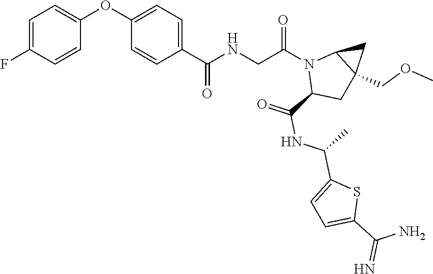 [SMILES: COC[C@@]12C[C@@H]1N(C(=O)CNC(=O)c1ccc(Oc3ccc(F)cc3)cc1)[C@H](C(=O)N[C@H](C)c1ccc(C(=N)N)s1)C2] | (1S,3S,5R)-N-(R)-1-(5- carbamimidoylthiophen- 2-yl)ethyl)-2-((4-(4- fluorophenoxy)benzoyl) glycyl)-5- (methoxymethyl)-2- azabicyclo[3.1.0]hexane- 3-carboxamide | *** | 1.43 | 594 |
| 39 | 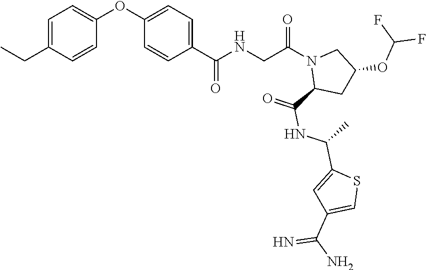 [SMILES: CCc1ccc(Oc2ccc(C(=O)NCC(=O)N3C[C@H](OC(F)F)C[C@H]3C(=O)N[C@H](C)c3cc(C(=N)N)cs3)cc2)cc1] | (2S,4R)-N-((R)-1-(4- carbamimidoylthiophen- 2-yl)ethyl)-4- (difluoromethoxy)-1-((4- (4- ethylphenoxy)benzoyl) glycyl)pyrrolidine-2- carboxamide | *** | 1.78 | 614 |
| 40 | 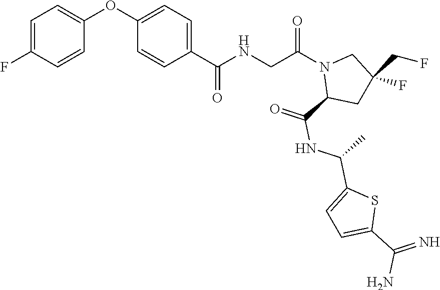 [SMILES: C[C@@H](NC(=O)[C@@H]1C[C@](F)(CF)CN1C(=O)CNC(=O)c1ccc(Oc2ccc(F)cc2)cc1)c1ccc(C(=N)N)s1] | (2S,4R)-N-((R)-1-(5- carbamimidoylthiophen- 2-yl)ethyl)-4-fluoro-4- (fluoromethyl)-1-((4-(4- fluorophenoxy)benzoyl) glycyl)pyrrolidine-2- carboxamide | *** | 1.46 | 588 |
| 41 | 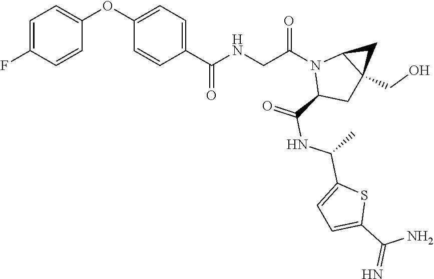 [SMILES: C[C@@H](NC(=O)[C@@H]1C[C@]2(CO)C[C@@H]2N1C(=O)CNC(=O)c1ccc(Oc2ccc(F)cc2)cc1)c1ccc(C(=N)N)s1] | (1S,3S,5R)-N-((R)-1-(5- carbamimidoylthiophen- 2-yl)ethyl)-2-((4-(4- fluorophenoxy)benzoyl) glycyl)-5- (hydroxymethyl)-2- azabicyclo[3.1.0]hexane- 3-carboxamide | *** | 1.31 | 580 |
| 42 | 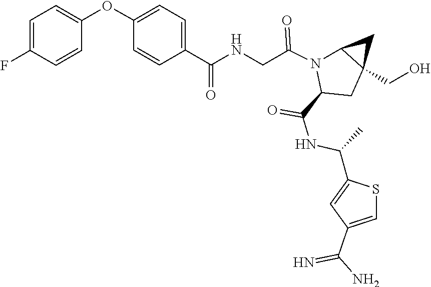 [SMILES: C[C@@H](NC(=O)[C@@H]1C[C@]2(CO)C[C@@H]2N1C(=O)CNC(=O)c1ccc(Oc2ccc(F)cc2)cc1)c1cc(C(=N)N)cs1] | (1S,3S,5R)-N-((R)-1-(4- carbamimidoylthiophen- 2-yl)ethyl)-2-((4-(4- fluorophenoxy)benzoyl) glycyl)-5- (hydroxymethyl)-2- azabicyclo[3.1.0]hexane- 3-carboxamide | *** | 1.33 | 580 |
| 43 | 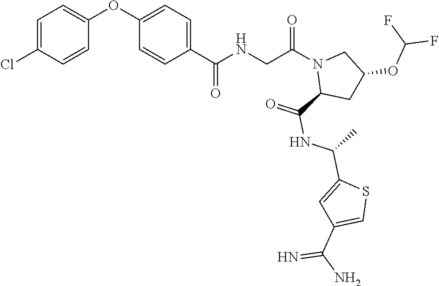 [SMILES: C[C@@H](NC(=O)[C@@H]1C[C@@H](OC(F)F)CN1C(=O)CNC(=O)c1ccc(Oc2ccc(Cl)cc2)cc1)c1cc(C(=N)N)cs1] | (2S,4R)-N-((R)-1-(4- carbamimidoylthiophen- 2-yl)ethyl)-1-((4-(4- chlorophenoxy)benzoyl) glycyl)-4- (difluoromethoxy) pyrrolidine-2- carboxamide | *** | 1.73 | 620 |
| 44 | 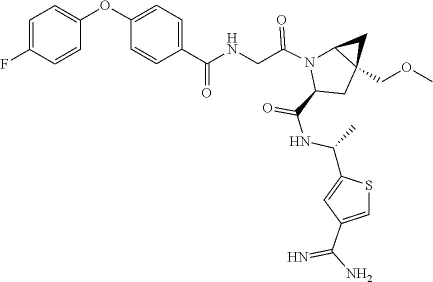 [SMILES: COC[C@@]12C[C@@H]1N(C(=O)CNC(=O)c1ccc(Oc3ccc(F)cc3)cc1)[C@H](C(=O)N[C@H](C)c1cc(C(=N)N)cs1)C2] | (1S,3S,5R)-N-((R)-1-(4- carbamimidoylthiophen- 2-yl)ethyl)-2-((4-(4- fluorophenoxy)benzoyl) glycyl)-5- (methoxymethyl)-2- azabicyclo[3.1.0]hexane- 3-carboxamide | *** | 1.49 | 594 |
| 45 | 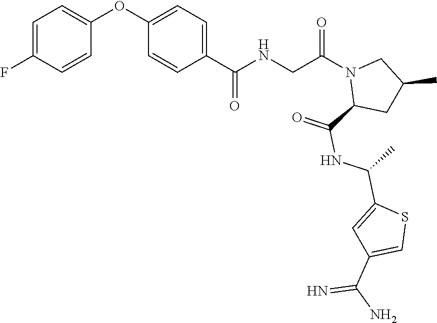 [SMILES: C[C@H]1C[C@@H](C(=O)N[C@H](C)c2cc(C(=N)N)cs2)N(C(=O)CNC(=O)c2ccc(Oc3ccc(F)cc3)cc2)C1] | (2S,4S)-N-((R)-1-(4- carbamimidoylthiophen- 2-yl)ethyl)-1-((4-(4- fluorophenoxy)benzoyl) glycyl)-4- methylpyrrolidine-2- carboxamide | *** | 1.50 | 552 |
| 46 | 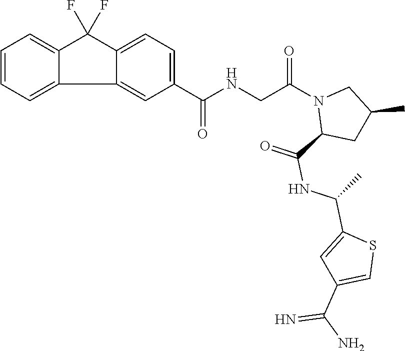 [SMILES: C[C@H]1C[C@@H](C(=O)N[C@H](C)c2cc(C(=N)N)cs2)N(C(=O)CNC(=O)c2ccc3c(c2)-c2ccccc2C3(F)F)C1] | (2S,4S)-N-((R)-1-(4- carbamimidoylthiophen- 2-yl)ethyl)-1-((9,9- difluoro-9H-fluorene-3- carbonyl)glycyl)-4- methylpyrrolidine-2- carboxamide | *** | 1.60 | 566 |
| 47 | 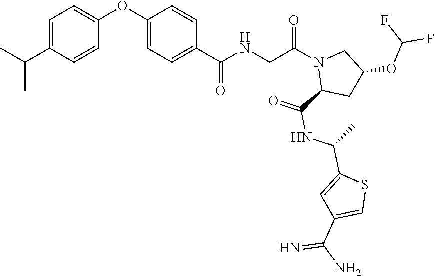 [SMILES: CC(C)c1ccc(Oc2ccc(C(=O)NCC(=O)N3C[C@H](OC(F)F)C[C@H]3C(=O)N[C@H](C)c3cc(C(=N)N)cs3)cc2)cc1] | (2S,4R)-N-((R)-1-(4- carbamimidoylthiophen- 2-yl)ethyl)-4- (difluoromethoxy)-1-((4- (4- isopropylphenoxy) benzoyl)glycyl) pyrrolidine-2- carboxamide | *** | 1.95 | 628 |
| 48 | 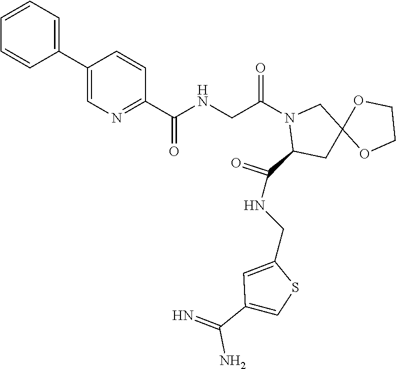 [SMILES: N=C(N)c1csc(CNC(=O)[C@@H]2CC3(CN2C(=O)CNC(=O)c2ccc(-c4ccccc4)cn2)OCCO3)c1] | (S)-N-((4- carbamimidoylthiophen- 2-yl)methyl)-7-((5- phenylpicolinoyl)glycyl)- 1,4-dioxa-7- azaspiro[4.4]nonane-8- carboxamide | *** | 1.29 | 549 |
| 49 | 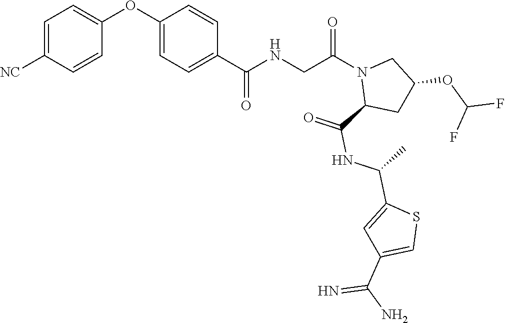 [SMILES: C[C@@H](NC(=O)[C@@H]1C[C@@H](OC(F)F)CN1C(=O)CNC(=O)c1ccc(Oc2ccc(C#N)cc2)cc1)c1cc(C(=N)N)cs1] | (2S,4R)-N-((R)-1-(4- carbamimidoylthiophen- 2-yl)ethyl)-1-((4-(4- cyanophenoxy)benzoyl) glycyl)-4- (difluoromethoxy) pyrrolidine-2- carboxamide | *** | 1.47 | 611 |
| 50 | 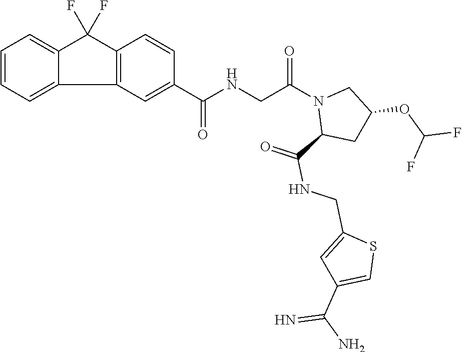 [SMILES: N=C(N)c1csc(CNC(=O)[C@@H]2C[C@@H](OC(F)F)CN2C(=O)CNC(=O)c2ccc3c(c2)-c2ccccc2C3(F)F)c1] | (2S,4R)-N-((4- carbamimidoylthiophen- 2-yl)methyl)-1-((9,9- difluoro-9H-fluorene-3- carbonyl)glycyl)-4- (difluoromethoxy) pyrrolidine-2- carboxamide | *** | 1.63 | 604 |
| 51 | 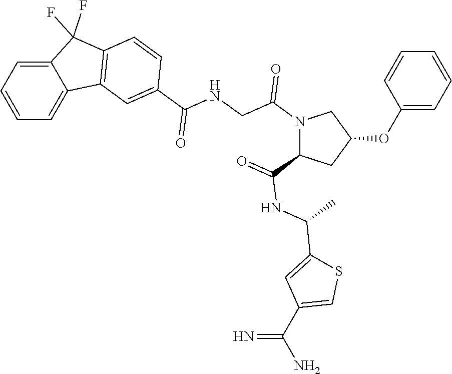 [SMILES: C[C@@H](NC(=O)[C@@H]1C[C@@H](Oc2ccccc2)CN1C(=O)CNC(=O)c1ccc2c(c1)-c1ccccc1C2(F)F)c1cc(C(=N)N)cs1] | (2S,4R)-N-((S)-1-(4- carbamimidoylthiophen- 2-yl)ethyl)-1-((9,9- difluoro-9H-fluorene-3- carbonyl)glycyl)-4- phenoxypyrrolidine-2- carboxamide | *** | 1.88 | 644 |
| 52 | 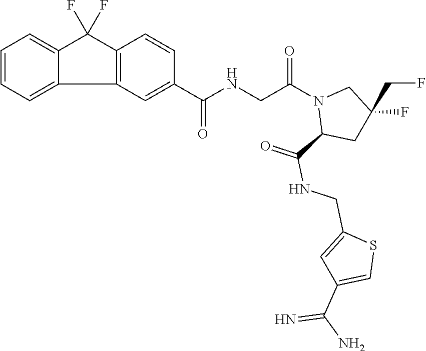 [SMILES: N=C(N)c1csc(CNC(=O)[C@@H]2C[C@](F)(CF)CN2C(=O)CNC(=O)c2ccc3c(c2)-c2ccccc2C3(F)F)c1] | (2S,4R)-N-((4- carbamimidoylthiophen- 2-yl)methyl)-1-((9,9- difluoro-9H-fluorene-3- carbonyl)glycyl)-4- fluoro-4- (fluoromethyl)pyrrolidine- 2-carboxamide | *** | 1.55 | 588 |
| 53 | 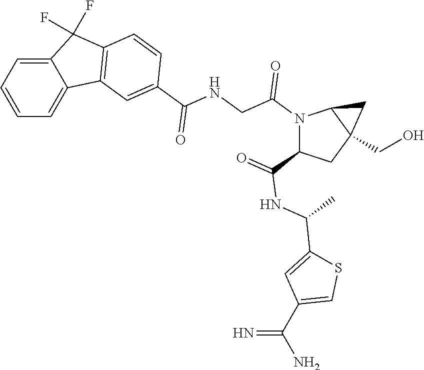 [SMILES: C[C@@H](NC(=O)[C@@H]1C[C@]2(CO)C[C@@H]2N1C(=O)CNC(=O)c1ccc2c(c1)-c1ccccc1C2(F)F)c1cc(C(=N)N)cs1] | (1S,3S,5R)-N-((R)-1-(4- carbamimidoylthiophen- 2-yl)ethyl)-2-((9,9- difluoro-9H-fluorene-3- carbonyl)glycyl)-5- (hydroxymethyl)-2- azabicyclo[3.1.0]hexane- 3-carboxamide | *** | 1.44 | 594 |
| 54 | 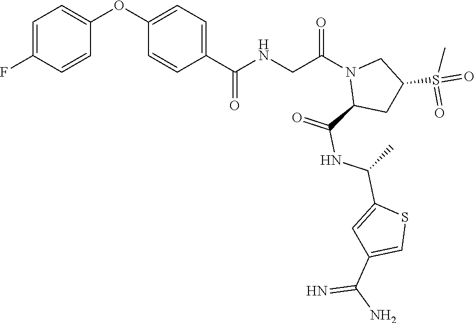 [SMILES: C[C@@H](NC(=O)[C@@H]1C[C@@H](S(C)(=O)=O)CN1C(=O)CNC(=O)c1ccc(Oc2ccc(F)cc2)cc1)c1cc(C(=N)N)cs1] | (2S,4R)-N-((R)-1-(4- carbamimidoylthiophen- 2-yl)ethyl)-1-((4-(4- fluorophenoxy)benzoyl) glycyl)-4- (methylsulfonyl) pyrrolidine- 2-carboxamide | *** | 1.38 | 616 |
| 55 | 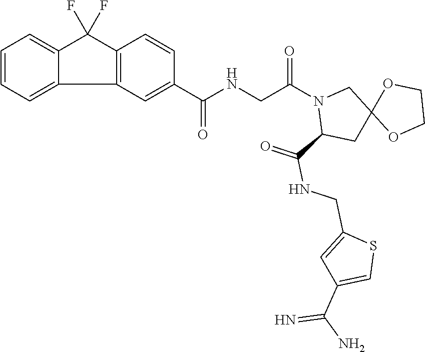 [SMILES: N=C(N)c1csc(CNC(=O)[C@@H]2CC3(CN2C(=O)CNC(=O)c2ccc4c(c2)-c2ccccc2C4(F)F)OCCO3)c1] | (S)-N-((4- carbamimidoylthiophen- 2-yl)methyl)-7-((9,9- difluoro-9H-fluorene-3- carbonyl)glycyl)-1,4- dioxa-7- azaspiro[4.4]nonane-8- carboxamide | *** | 1.48 | 596 |
| 56 | 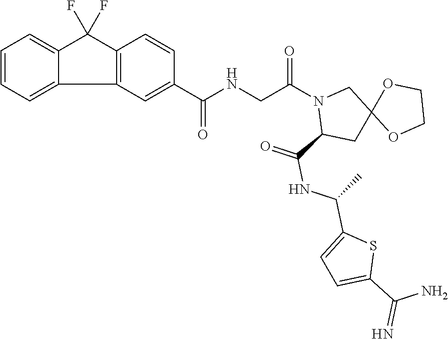 [SMILES: C[C@@H](NC(=O)[C@@H]1CC2(CN1C(=O)CNC(=O)c1ccc3c(c1)-c1ccccc1C3(F)F)OCCO2)c1ccc(C(=N)N)s1] | (S)-N-((R)-1-(5- carbamimidoylthiophen- 2-yl)ethyl)-7-((9,9- difluoro-9H-fluorene-3- carbonyl)glycyl)-1,4- dioxa-7- azaspiro[4.4]nonane-8- carboxamide | *** | 1.50 | 610 |
| 57 | 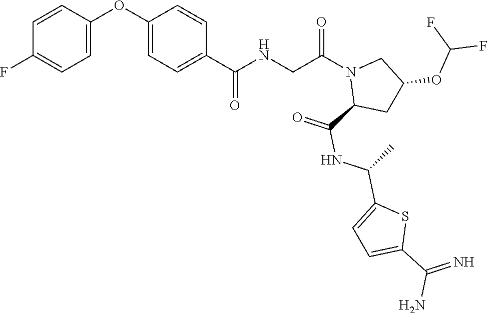 [SMILES: C[C@@H](NC(=O)[C@@H]1C[C@@H](OC(F)F)CN1C(=O)CNC(=O)c1ccc(Oc2ccc(F)cc2)cc1)c1ccc(C(=N)N)s1] | (2S,4R)-N-((R)-1-(5- carbamimidoylthiophen- 2-yl)ethyl)-4- (difluoromethoxy)-1-((4- (4- fluorophenoxy)benzoyl) glycyl)pyrrolidine-2- carboxamide | *** | 1.59 | 604 |
| 58 | 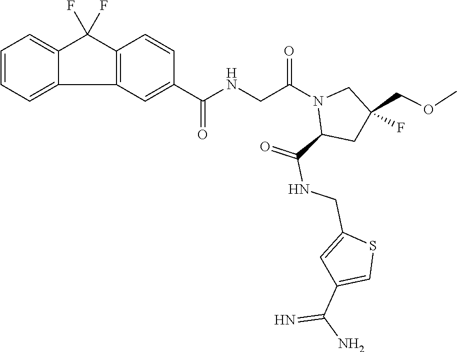 [SMILES: COC[C@@]1(F)C[C@@H](C(=O)NCc2cc(C(=N)N)cs2)N(C(=O)CNC(=O)c2ccc3c(c2)-c2ccccc2C3(F)F)C1] | (2S,4R)-N-((4- carbamimidoylthiophen- 2-yl)methyl)-1-((9,9- difluoro-9H-fluorene-3- carbonyl)glycyl)-4- fluoro-4- (methoxymethyl) pyrrolidine-2- carboxamide | *** | 1.54 | 600 |
| 59 | 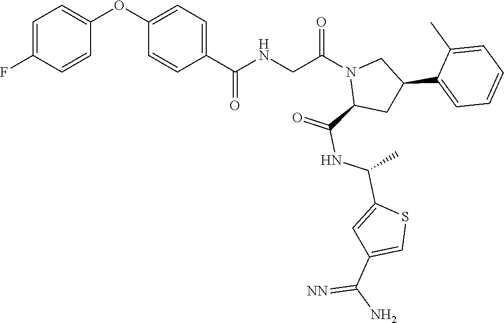 | (2S,4R)-N-((R)-1-(4- carbamimidoylthiophen- 2-yl)ethyl)-1-((4-(4- fluorophenoxy)benzoyl) glycyl)-4-(o- tolyl)pyrrolidine-2- carboxamide | *** | 1.90 | 628 |
| 60 | 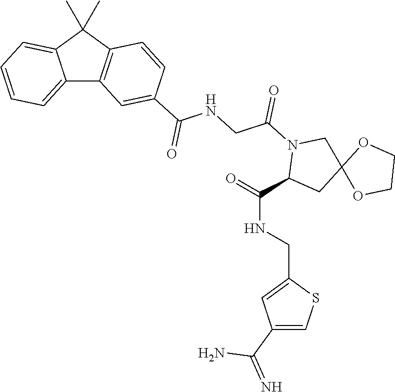 [SMILES: CC1(C)c2ccccc2-c2cc(C(=O)NCC(=O)N3CC4(C[C@H]3C(=O)NCc3cc(C(=N)N)cs3)OCCO4)ccc21] | (S)-N-((4- carbamimidoylthiophen- 2-yl)methyl)-7-((9,9- dimethyl-9H-fluorene-3- carbonyl)glycyl)-1,4- dioxa-7- azaspiro[4.4]nonane-8- carboxamide | *** | 1.57 | 588 |
| 61 | 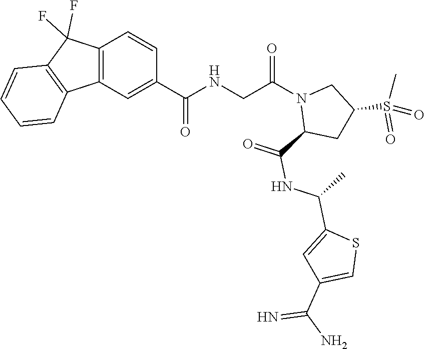 [SMILES: C[C@@H](NC(=O)[C@@H]1C[C@@H](S(C)(=O)=O)CN1C(=O)CNC(=O)c1ccc2c(c1)-c1ccccc1C2(F)F)c1cc(C(=N)N)cs1] | (2S,4R)-N-((R)-1-(4- carbamimidoylthiophen- 2-yl)ethyl)-1-((9,9- difluoro-9H-fluorene-3- carbonyl)glycyl)-4- (methylsulfonyl) pyrrolidine-2- carboxamide | *** | 1.42 | 630 |
| 62 | 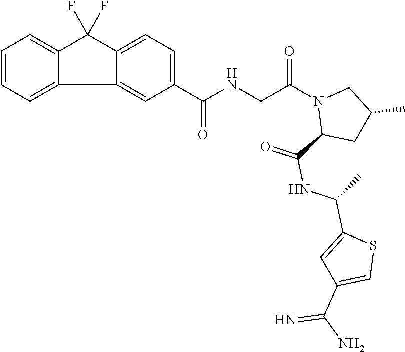 [SMILES: C[C@@H]1C[C@@H](C(=O)N[C@H](C)c2cc(C(=N)N)cs2)N(C(=O)CNC(=O)c2ccc3c(c2)-c2ccccc2C3(F)F)C1] | (2S,4R)-N-((R)-1-(4- carbamimidoylthiophen- 2-yl)ethyl)-1-((9,9- difluoro-9H-fluorene-3- carbonyl)glycyl)-4- methylpyrrolidine-2- carboxamide | *** | 1.61 | 566 |
| 63 | 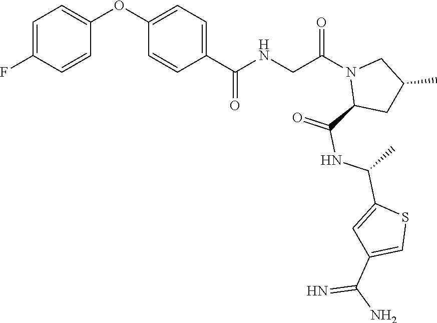 [SMILES: C[C@@H]1C[C@@H](C(=O)N[C@H](C)c2cc(C(=N)N)cs2)N(C(=O)CNC(=O)c2ccc(Oc3ccc(F)cc3)cc2)C1] | (2S,4R)-N-((R)-1-(4- carbamimidoylthiophen- 2-yl)ethyl)-1-((4-(4- fluorophenoxy)benzoyl) glycyl)-4- methylpyrrolidine-2- carboxamide | *** | 1.52 | 552 |
| 64 | 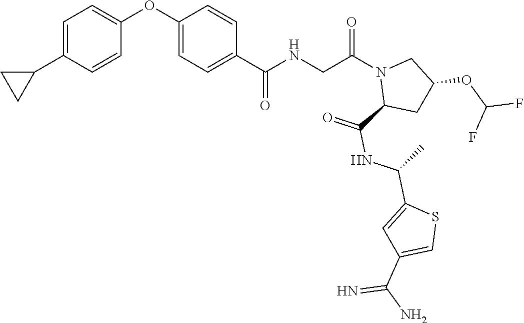 [SMILES: C[C@@H](NC(=O)[C@@H]1C[C@@H](OC(F)F)CN1C(=O)CNC(=O)c1ccc(Oc2ccc(C3CC3)cc2)cc1)c1cc(C(=N)N)cs1] | (2S,4R)-N-((R)-1-(4- carbamimidoylthiophen- 2-yl)ethyl)-1-((4-(4- cyclopropylphenoxy) benzoyl)glycyl)-4- (difluoromethoxy) pyrrolidine-2- carboxamide | *** | 1.82 | 626 |
| 65 | 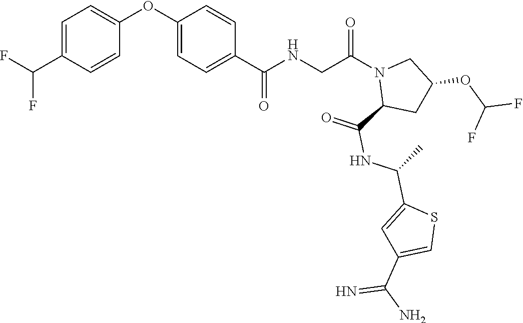 [SMILES: C[C@@H](NC(=O)[C@@H]1C[C@@H](OC(F)F)CN1C(=O)CNC(=O)c1ccc(Oc2ccc(C(F)F)cc2)cc1)c1cc(C(=N)N)cs1] | (2S,4R)-N-((R)-1-(4- carbamimidoylthiophen- 2-yl)ethyl)-4- (difluoromethoxy)-1-((4- (4- (difluoromethyl)phenoxy) benzoyl)glycyl) pyrrolidine-2- carboxamide | *** | 1.63 | 636 |
| 66 | 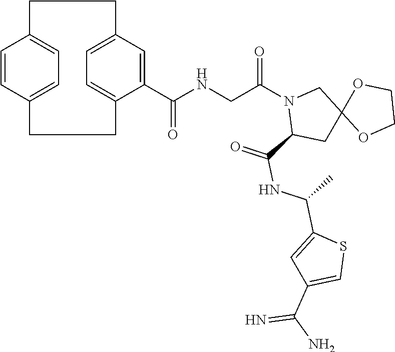 [SMILES: C[C@@H](NC(=O)[C@@H]1CC2(CN1C(=O)CNC(=O)c1cc3ccc1CCc1ccc(cc1)CC3)OCCO2)c1cc(C(=N)N)cs1] | (S)-7-((1,4(1,4)- dibenzenacyclo- hexaphane-12- carbonyl)glycyl)-N-((R)- 1-(4- carbamimidoylthiophen- 2-yl)ethyl)-1,4-dioxa-7- azaspiro[4.4]nonane-8- carboxamide | *** | 1.46 | 616 |
| 67 | 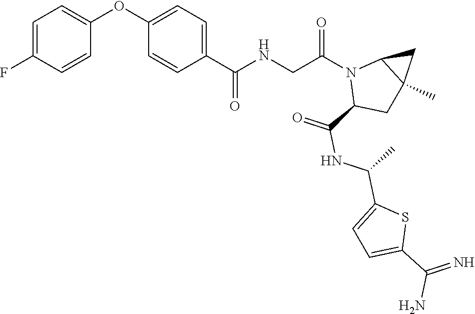 [SMILES: C[C@@H](NC(=O)[C@@H]1C[C@]2(C)C[C@@H]2N1C(=O)CNC(=O)c1ccc(Oc2ccc(F)cc2)cc1)c1ccc(C(=N)N)s1] | (1S,3S,5S)-N-((R)-1-(5- carbamimidoylthiophen- 2-yl)ethyl)-2-((4-(4- fluorophenoxy)benzoyl) glycyl)-5-methyl-2- azabicyclo[3.1.0]hexane- 3-carboxamide | *** | 1.56 | 564 |
| 68 | 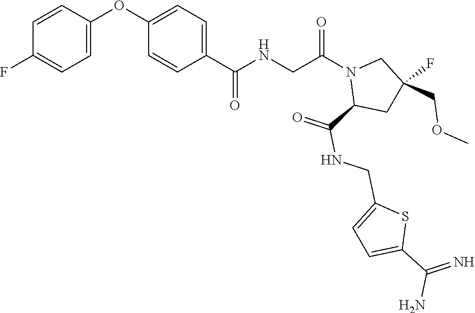 [SMILES: COC[C@@]1(F)C[C@@H](C(=O)NCc2ccc(C(=N)N)s2)N(C(=O)CNC(=O)c2ccc(Oc3ccc(F)cc3)cc2)C1] | (2S,4R)-N-((5- carbamimidoylthiophen- 2-yl)methyl)-4-fluoro-1- ((4-(4- fluorophenoxy)benzoyl) glycyl)-4- (methoxymethyl) pyrrolidine-2- carboxamide | *** | 1.40 | 586 |
| 69 | 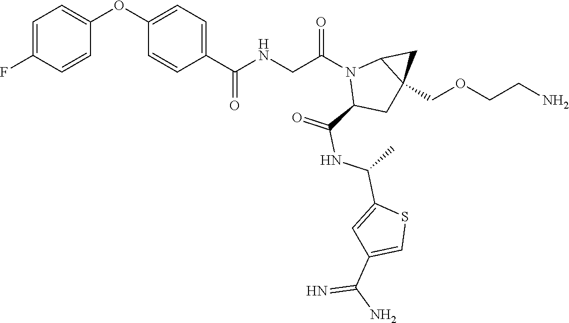 | (1S,3S,5R)-5-((2- aminoethoxy)methyl)- N-((R)-1-(4- carbamimidoylthiophen- 2-yl)ethyl)-2-((4-(4- fluorophenoxy)benzoyl) glycyl)-2- azabicyclo[3.1.0]hexane- 3-carboxamide | *** | 1.00 | 623 |
| 70 | 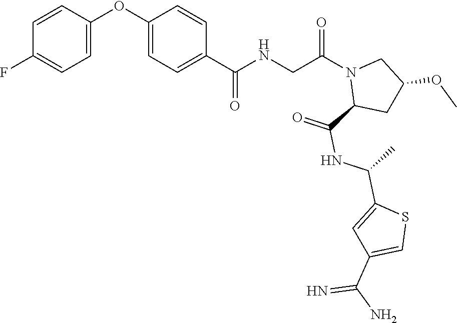 [SMILES: CO[C@@H]1C[C@@H](C(=O)N[C@H](C)c2cc(C(=N)N)cs2)N(C(=O)CNC(=O)c2ccc(Oc3ccc(F)cc3)cc2)C1] | (2S,4R)-N-((R)-1-(4- carbamimidoylthiophen- 2-yl)ethyl)-1-((4-(4- fluorophenoxy)benzoyl) glycyl)-4- methoxypyrrolidine-2- carboxamide | *** | 1.38 | 568 |
| 71 | 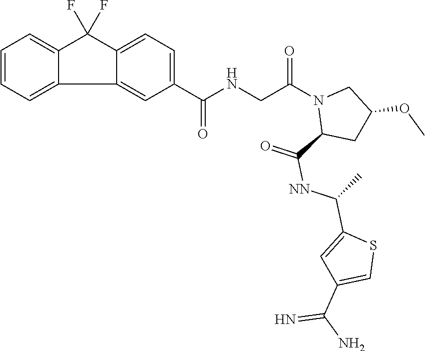 | (2S,4R)-N-((R)-1-(4- carbamimidoylthiophen- 2-yl)ethyl)-1-((9,9- difluoro-9H-fluorene-3- carbonyl)glycyl)-4- methoxypyrrolidine-2- carboxamide | *** | 1.45 | 582 |
| 72 | 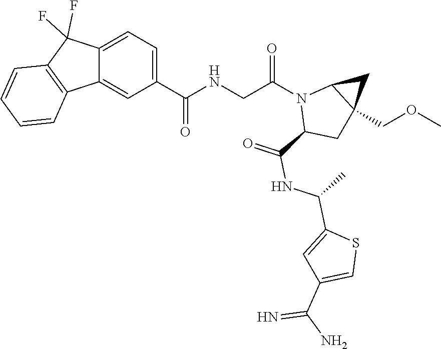 [SMILES: COC[C@@]12C[C@@H]1N(C(=O)CNC(=O)c1ccc3c(c1)-c1ccccc1C3(F)F)[C@H](C(=O)N[C@H](C)c1cc(C(=N)N)cs1)C2] | (1S,3S,5R)-N-((R)-1-(4- carbamimidoylthiophen- 2-yl)ethyl)-2-((9,9- difluoro-9H-fluorene-3- carbonyl)glycyl)-5- (methoxymethyl)-2- azabicyclo[3.1.0]hexane- 3-carboxamide | *** | 1.57 | 608 |
| 73 | 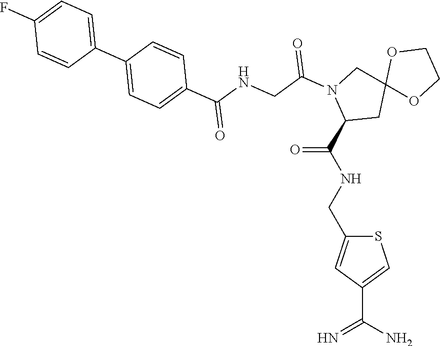 [SMILES: N=C(N)c1csc(CNC(=O)[C@@H]2CC3(CN2C(=O)CNC(=O)c2ccc(-c4ccc(F)cc4)cc2)OCCO3)c1] | (S)-N-((4- carbamimidoylthiophen- 2-yl)methyl)-7-((4′- fluoro-[1,1′-biphenyl]-4- carbonyl)glycyl)-1,4- dioxa-7- azaspiro[4.4]nonane-8- carboxamide | *** | 1.49 | 566 |
| 74 | 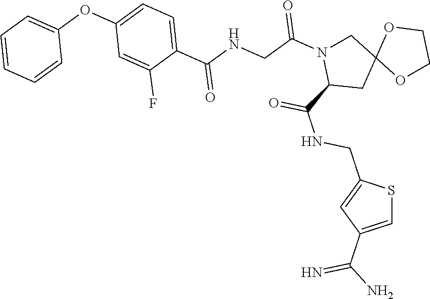 [SMILES: N=C(N)c1csc(CNC(=O)[C@@H]2CC3(CN2C(=O)CNC(=O)c2ccc(Oc4ccccc4)cc2F)OCCO3)c1] | (S)-N-((4- carbamimidoylthiophen- 2-yl)methyl)-7-((2- fluoro-4- phenoxybenzoyl)glycyl)- 1,4-dioxa-7- azaspiro[4.4]nonane-8- carboxamide | *** | 1.52 | 582 |
| 75 | 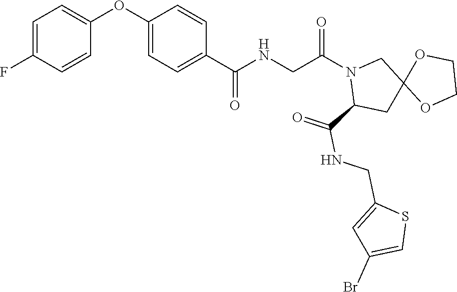 [SMILES: O=C(NCC(=O)N1CC2(C[C@H]1C(=O)NCc1cc(Br)cs1)OCCO2)c1ccc(Oc2ccc(F)cc2)cc1] | (S)-N-((4- bromothiophen-2- yl)methyl)-7-((4-(4- fluorophenoxy)benzoyl) glycyl)-1,4-dioxa-7- azaspiro[4.4]nonane-8- carboxamide | * | 2.64 | 620 |
| 76 | 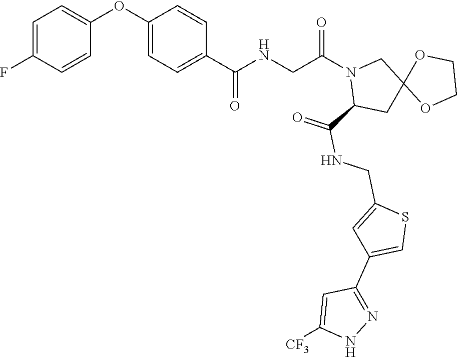 [SMILES: O=C(NCC(=O)N1CC2(C[C@H]1C(=O)NCc1cc(-c3cc(C(F)(F)F)[nH]n3)cs1)OCCO2)c1ccc(Oc2ccc(F)cc2)cc1] | (S)-7-((4-(4- fluorophenoxy)benzoyl) glycyl)-N-((4-(5- (trifluoromethyl)-1H- pyrazol-3-yl)thiophen-2- yl)methyl)-1,4-dioxa-7- azaspiro[4.4]nonane-8- carboxamide | * | 2.66 | 674 |
| 77 | 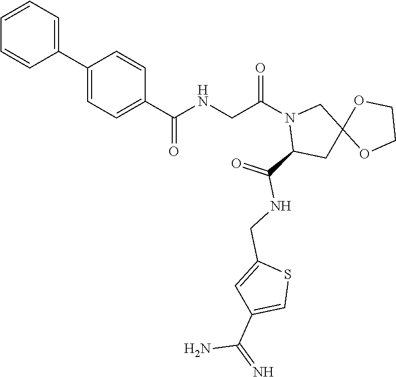 [SMILES: N=C(N)c1csc(CNC(=O)[C@@H]2CC3(CN2C(=O)CNC(=O)c2ccc(-c4ccccc4)cc2)OCCO3)c1] | (S)-7-(([1,1′-biphenyl]- 4-carbonyl)glycyl)-N- ((4- carbamimidoylthiophen- 2-yl)methyl)-1,4-dioxa- 7-azaspiro[4.4]nonane- 8-carboxamide | *** | 1.42 | 548 |
| 78 | 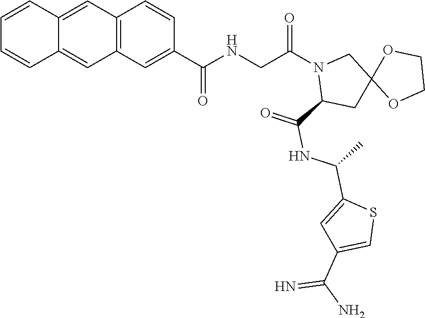 [SMILES: C[C@@H](NC(=O)[C@@H]1CC2(CN1C(=O)CNC(=O)c1ccc3cc4ccccc4cc3c1)OCCO2)c1cc(C(=N)N)cs1] | (S)-7-((anthracene-2- carbonyl)glycyl)-N-((R)- 1-(4- carbamimidoylthiophen- 2-yl)ethyl)-1,4-dioxa-7- azaspiro[4.4]nonane-8- carboxamide | *** | 1.51 | 586 |
| 79 | 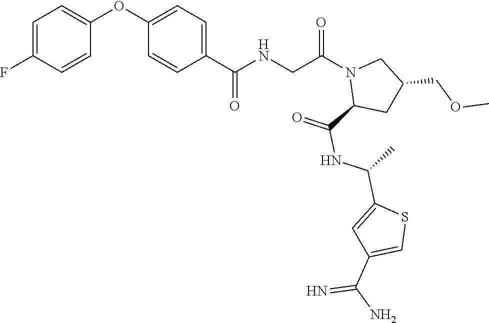 [SMILES: COC[C@@H]1C[C@@H](C(=O)N[C@H](C)c2cc(C(=N)N)cs2)N(C(=O)CNC(=O)c2ccc(Oc3ccc(F)cc3)cc2)C1] | (2S,4R)-N-((R)-1-(4- carbamimidoylthiophen- 2-yl)ethyl)-1-((4-(4- fluorophenoxy)benzoyl) glycyl)-4- (methoxymethyl) pyrrolidine-2- carboxamide | *** | 1.34 | 582 |
| 80 | 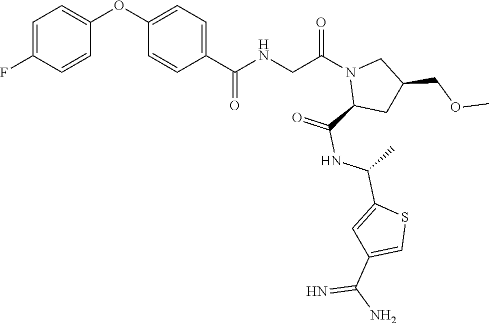 [SMILES: COC[C@H]1C[C@@H](C(=O)N[C@H](C)c2cc(C(=N)N)cs2)N(C(=O)CNC(=O)c2ccc(Oc3ccc(F)cc3)cc2)C1] | (2S,4S)-N-((R)-1-(4- carbamimidoylthiophen- 2-yl)ethyl)-1-((4-(4- fluorophenoxy)benzoyl) glycyl)-4- (methoxymethyl) pyrrolidine-2- carboxamide | *** | 1.37 | 582 |
| 81 | 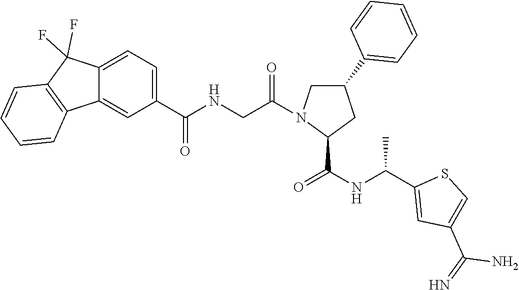 [SMILES: C[C@@H](NC(=O)[C@@H]1C[C@@H](c2ccccc2)CN1C(=O)CNC(=O)c1ccc2c(c1)-c1ccccc1C2(F)F)c1cc(C(=N)N)cs1] | (2S,4S)-N-((R)-1-(4- carbamimidoylthiophen- 2-yl)ethyl)-1-((9,9- difluoro-9H-fluorene-3- carbonyl)glycyl)-4- phenylpyrrolidine-2- carboxamide | *** | 1.87 | 628 |
| 82 | 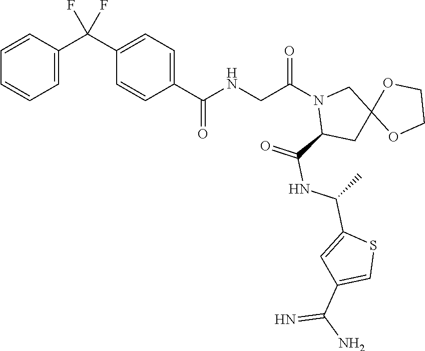 [SMILES: C[C@@H](NC(=O)[C@@H]1CC2(CN1C(=O)CNC(=O)c1ccc(C(F)(F)c3ccccc3)cc1)OCCO2)c1cc(C(=N)N)cs1] | (S)-N-((R)-1-(4- carbamimidoylthiophen- 2-yl)ethyl)-7-((4- (difluoro(phenyl)methyl) benzoyl)glycyl)-1,4- dioxa-7- azaspiro[4.4]nonane-8- carboxamide | *** | 1.47 | 612 |
| 83 | 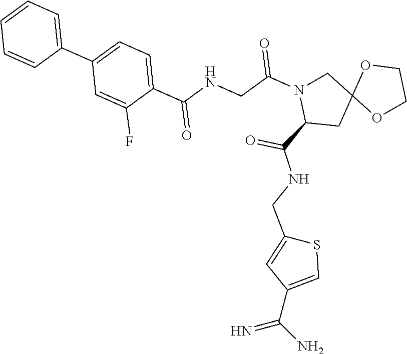 [SMILES: N=C(N)c1csc(CNC(=O)[C@@H]2CC3(CN2C(=O)CNC(=O)c2ccc(-c4ccccc4)cc2F)OCCO3)c1] | (S)-N-((4- carbamimidoylthiophen- 2-yl)methyl)-7-((3- fluoro-[1,1′-biphenyl]-4- carbonyl)glycyl)-1,4- dioxa-7- azaspiro[4.4]nonane-8- carboxamide | *** | 1.47 | 566 |
| 84 | 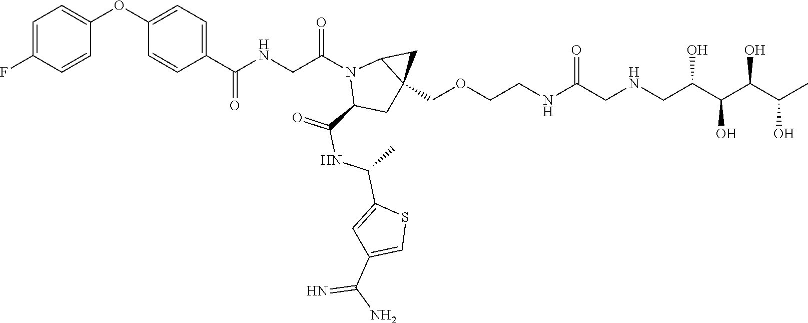 | (1S,3S,5R)-N-((R)-1-(4- carbamimidoylthiophen- 2-yl)ethyl)-2-((4-(4- fluorophenoxy)benzoyl) glycyl)-5-((2-(2- (((2S,3S,4S,5S)- 2,3,4,5- tetrahydroxyhexyl)amino) acetamido)ethoxy) methyl)-2- azabicyclo[3.1.0]hexane- 3-carboxamide | *** | 0.87 | 828 |
| 85 | 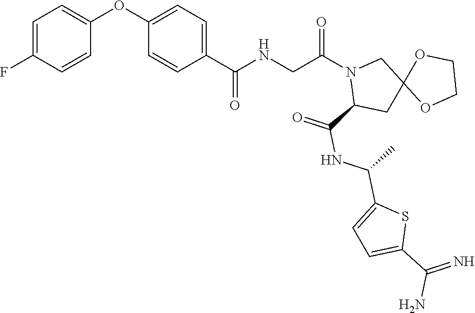 [SMILES: C[C@@H](NC(=O)[C@@H]1CC2(CN1C(=O)CNC(=O)c1ccc(Oc3ccc(F)cc3)cc1)OCCO2)c1ccc(C(=N)N)s1] | (S)-N-((R)-1-(5- carbamimidoylthiophen- 2-yl)ethyl)-7-((4-(4- fluorophenoxy)benzoyl) glycyl)-1,4-dioxa-7- azaspiro[4.4]nonane-8- carboxamide | *** | 1.33 | 596 |
| 86 | 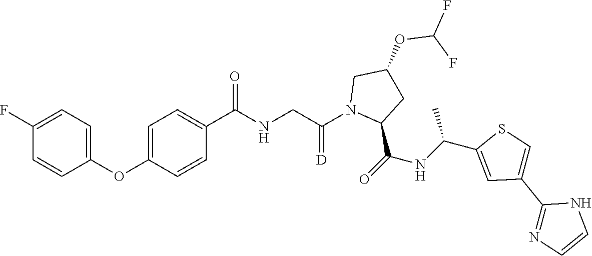 | (2S,4R)-N-((R)-1-(4- (1H-imidazol-2- yl)thiophen-2-yl)ethyl)- 4-(difluoromethoxy)-1- ((4-(4- fluorophenoxy)benzoyl) glycyl)pyrrolidine-2- carboxamide | *** | 1.53 | 628 |
| 87 | 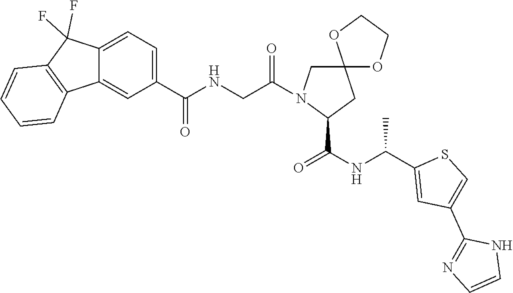 [SMILES: C[C@@H](NC(=O)[C@@H]1CC2(CN1C(=O)CNC(=O)c1ccc3c(c1)-c1ccccc1C3(F)F)OCCO2)c1cc(-c2ncc[nH]2)cs1] | (S)-N-((R)-1-(4-(1H- imidazol-2-yl)thiophen- 2-yl)ethyl)-7-((9,9- difluoro-9H-fluorene-3- carbonyl)glycyl)-1,4- dioxa-7- azaspiro[4.4]nonane-8- carboxamide | *** | 1.45 | 634 |
| 88 | 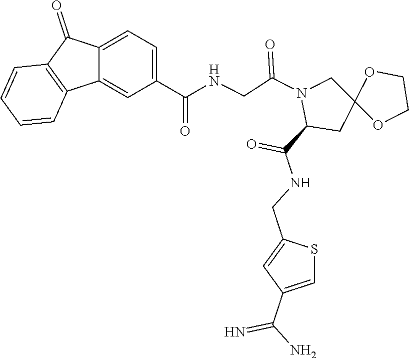 [SMILES: N=C(N)c1csc(CNC(=O)[C@@H]2CC3(CN2C(=O)CNC(=O)c2ccc4c(c2)-c2ccccc2C4=O)OCCO3)c1] | (S)-N-((4- carbamimidoylthiophen- 2-yl)methyl)-7-((9-oxo- 9H-fluorene-3- carbonyl)glycyl)-1,4- dioxa-7- azaspiro[4.4]nonane-8- carboxamide | *** | 1.10 | 574 |
| 89 | 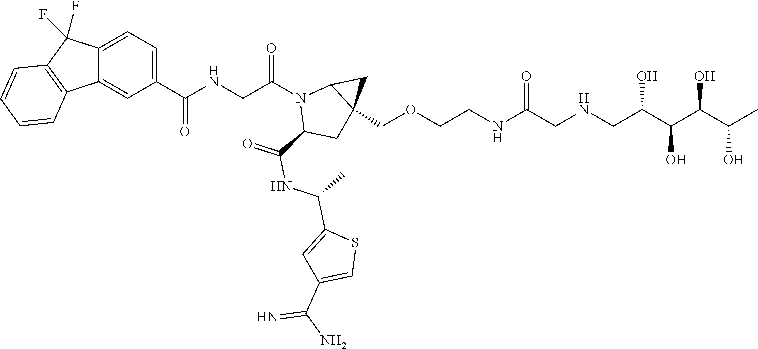 | (1S,3S,5R)-N-((R)-1-(4- carbamimidoylthiophen- 2-yl)ethyl)-2-((9,9- difluoro-9H-fluorene-3- carbonyl)glycyl)-5-((2- (2-(((2S,3S,4S,5S)- 2,3,4,5- tetrahydroxyhexyl)amino) acetamido)ethoxy) methyl)-2- azabicyclo[3.1.0]hexane- 3-carboxamide | *** | 0.94 | 842 |
| 90 | 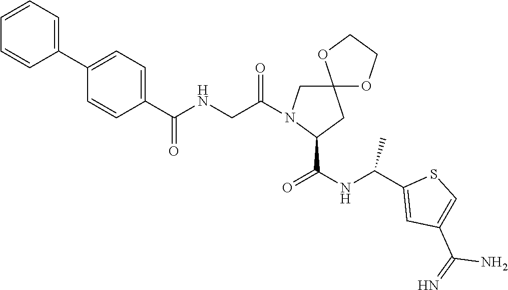 [SMILES: C[C@@H](NC(=O)[C@@H]1CC2(CN1C(=O)CNC(=O)c1ccc(-c3ccccc3)cc1)OCCO2)c1cc(C(=N)N)cs1] | (S)-7-(([1,1′-biphenyl]- 4-carbonyl)glycyl)-N- ((R)-1-(4- carbamimidoylthiophen- 2-yl)ethyl)-1,4-dioxa-7- azaspiro[4.4]nonane-8- carboxamide | *** | 1.20 (A) | 562 |
| 91 | 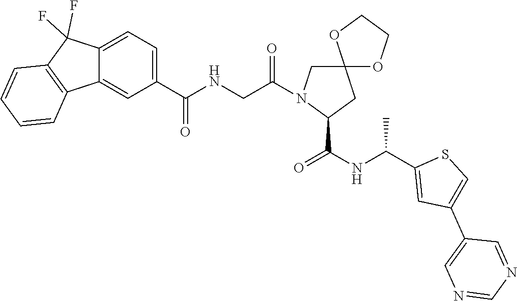 [SMILES: C[C@@H](NC(=O)[C@@H]1CC2(CN1C(=O)CNC(=O)c1ccc3c(c1)-c1ccccc1C3(F)F)OCCO2)c1cc(-c2cncnc2)cs1] | (S)-7-((9,9-difluoro-9H- fluorene-3- carbonyl)glycyl)-N-((R)- 1-(4-(pyrimidin-5- yl)thiophen-2-yl)ethyl)- 1,4-dioxa-7- azaspiro[4.4]nonane-8- carboxamide | * | 2.12 | 646 |
| 92 | 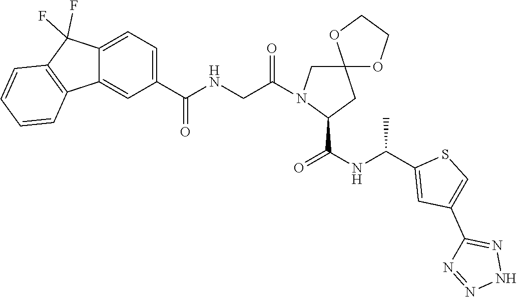 [SMILES: C[C@@H](NC(=O)[C@@H]1CC2(CN1C(=O)CNC(=O)c1ccc3c(c1)-c1ccccc1C3(F)F)OCCO2)c1cc(-c2nn[nH]n2)cs1] | (S)-N-((R)-1-(4-(2H- tetrazol-5-yl)thiophen- 2-yl)ethyl)-7-((9,9- difluoro-9H-fluorene-3- carbonyl)glycyl)-1,4- dioxa-7- azaspiro[4.4]nonane-8- carboxamide | * | 2.03 | 636 |
| 93 | 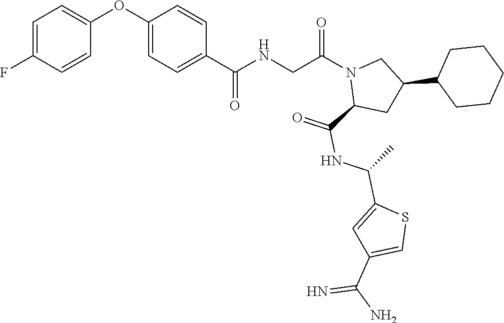 [SMILES: C[C@@H](NC(=O)[C@@H]1C[C@H](C2CCCCC2)CN1C(=O)CNC(=O)c1ccc(Oc2ccc(F)cc2)cc1)c1cc(C(=N)N)cs1] | (2S,4R)-N-((R)-1-(4- carbamimidoylthiophen- 2-yl)ethyl)-4- cyclohexyl-1-((4-(4- fluorophenoxy)benzoyl) glycyl)pyrrolidine-2- carboxamide | *** | 1.83 | 620 |
| 94 | 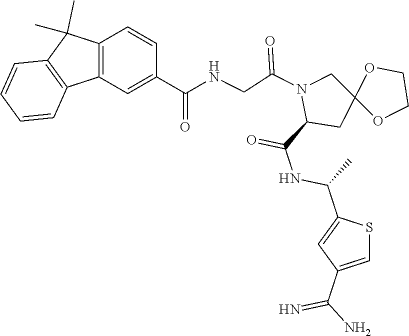 [SMILES: C[C@@H](NC(=O)[C@@H]1CC2(CN1C(=O)CNC(=O)c1ccc3c(c1)-c1ccccc1C3(C)C)OCCO2)c1cc(C(=N)N)cs1] | (S)-N-((R)-1-(4- carbamimidoylthiophen- 2-yl)ethyl)-7-((9,9- dimethyl-9H-fluorene-3- carbonyl)glycyl)-1,4- dioxa-7- azaspiro[4.4]nonane-8- carboxamide | *** | 1.47 | 602 |
| 95 | 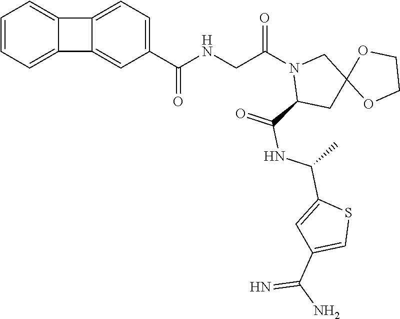 [SMILES: C[C@@H](NC(=O)[C@@H]1CC2(CN1C(=O)CNC(=O)c1ccc3c(c1)-c1ccccc1-3)OCCO2)c1cc(C(=N)N)cs1] | (S)-7-((biphenylene-2- carbonyl)glycyl)-N-((R)- 1-(4- carbamimidoylthiophen- 2-yl)ethyl)-1,4-dioxa-7- azaspiro[4.4]nonane-8- carboxamide | *** | 1.18 (A) | 560 |
| 96 | 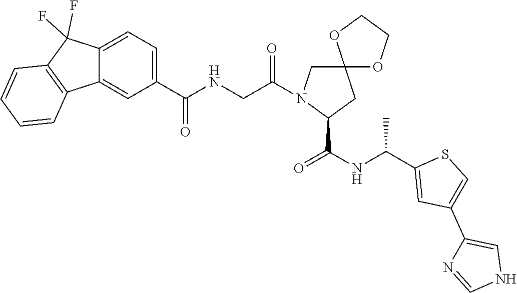 [SMILES: C[C@@H](NC(=O)[C@@H]1CC2(CN1C(=O)CNC(=O)c1ccc3c(c1)-c1ccccc1C3(F)F)OCCO2)c1cc(-c2c[nH]cn2)cs1] | (S)-N-((R)-1-(4-(1H- imidazol-4-yl)thiophen- 2-yl)ethyl)-7-((9,9- difluoro-9H-fluorene-3- carbonyl)glycyl)-1,4- dioxa-7- azaspiro[4.4]nonane-8- carboxamide | ** | 1.42 | 634 |
| 97 | 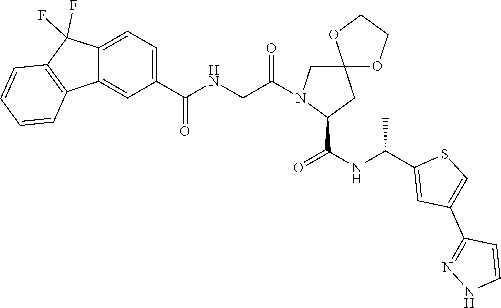 [SMILES: C[C@@H](NC(=O)[C@@H]1CC2(CN1C(=O)CNC(=O)c1ccc3c(c1)-c1ccccc1C3(F)F)OCCO2)c1cc(-c2cc[nH]n2)cs1] | (S)-N-((R)-1-(4-(1H- pyrazol-3-yl)thiophen-2- yl)ethyl)-7-((9,9- difluoro-9H-fluorene-3- carbonyl)glycyl)-1,4- dioxa-7- azaspiro[4.4]nonane-8- carboxamide | * | 2.17 | 634 |
| 98 | 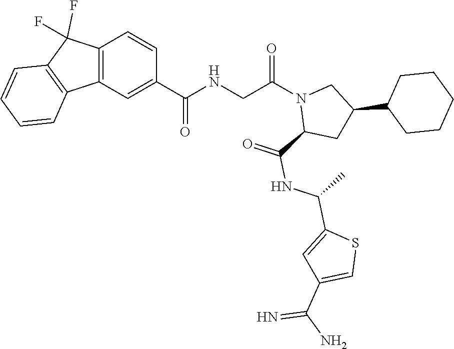 [SMILES: C[C@@H](NC(=O)[C@@H]1C[C@H](C2CCCCC2)CN1C(=O)CNC(=O)c1ccc2c(c1)-c1ccccc1C2(F)F)c1cc(C(=N)N)cs1] | (2S,4R)-N-((R)-1-(4- carbamimidoylthiophen- 2-yl)ethyl)-4- cyclohexyl-1-((9,9- difluoro-9H-fluorene-3- carbonyl)glycyl) pyrrolidine-2- carboxamide | *** | 1.91 | 634 |
| 99 | 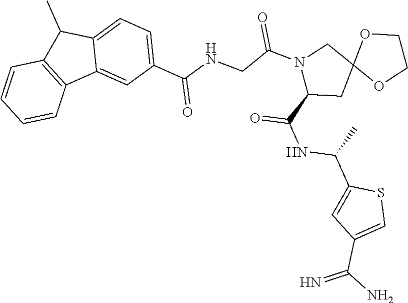 [SMILES: CC1c2ccccc2-c2cc(C(=O)NCC(=O)N3CC4(C[C@H]3C(=O)N[C@H](C)c3cc(C(=N)N)cs3)OCCO4)ccc21] | (8S)-N-((R)-1-(4- carbamimidoylthiophen- 2-yl)ethyl)-7-((9- methyl-9H-fluorene-3- carbonyl)glycyl)-1,4- dioxa-7- azaspiro[4.4]nonane-8- carboxamide | *** | 1.41 | 588 |
| 100 | 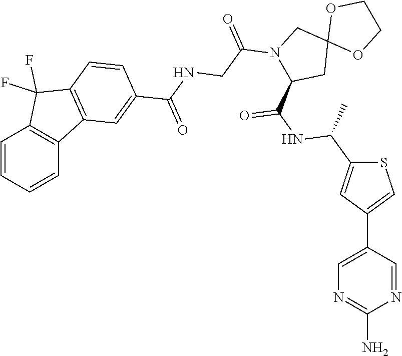 [SMILES: C[C@@H](NC(=O)[C@@H]1CC2(CN1C(=O)CNC(=O)c1ccc3c(c1)-c1ccccc1C3(F)F)OCCO2)c1cc(-c2cnc(N)nc2)cs1] | (S)-N-((R)-1-(4-(2- aminopyrimidin-5- yl)thiophen-2-yl)ethyl)- 7-((9,9-difluoro-9H- fluorene-3- carbonyl)glycyl)-1,4- dioxa-7- azaspiro[4.4]nonane-8- carboxamide | * | 2.01 | 661 |
| 101 | 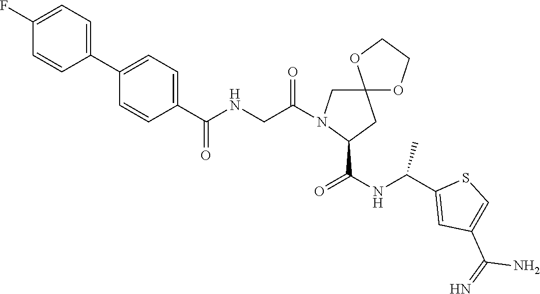 [SMILES: C[C@@H](NC(=O)[C@@H]1CC2(CN1C(=O)CNC(=O)c1ccc(-c3ccc(F)cc3)cc1)OCCO2)c1cc(C(=N)N)cs1] | (S)-N-((R)-1-(4- carbamimidoylthiophen- 2-yl)ethyl)-7-((4′-fluoro- [1,1′-biphenyl]-4- carbonyl)glycyl)-1,4- dioxa-7- azaspiro[4.4]nonane-8- carboxamide | *** | 1.31 | 580 |
| 102 | 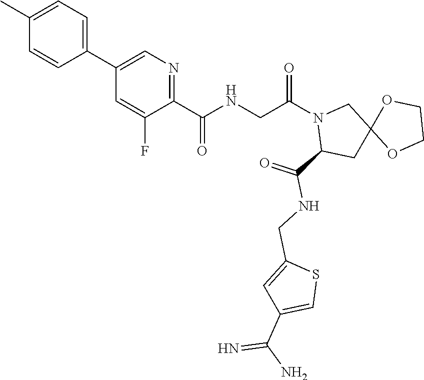 [SMILES: Cc1ccc(-c2cnc(C(=O)NCC(=O)N3CC4(C[C@H]3C(=O)NCc3cc(C(=N)N)cs3)OCCO4)c(F)c2)cc1] | (S)-N-((4- carbamimidoylthiophen- 2-yl)methyl)-7-((3- fluoro-5-(p- tolyl)picolinoyl)glycyl)- 1,4-dioxa-7- azaspiro[4.4]nonane-8- carboxamide | *** | 1.45 | 581 |
| 103 | 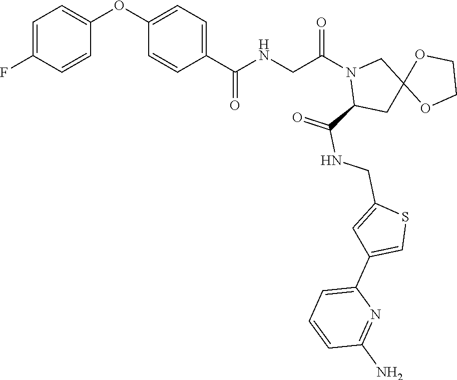 [SMILES: Nc1cccc(-c2csc(CNC(=O)[C@@H]3CC4(CN3C(=O)CNC(=O)c3ccc(Oc5ccc(F)cc5)cc3)OCCO4)c2)n1] | (S)-N-((4-(6- aminopyridin-2- yl)thiophen-2- yl)methyl)-7-((4-(4- fluorophenoxy)benzoyl) glycyl)-1,4-dioxa-7- azaspiro[4.4]nonane-8- carboxamide | * | | |
| 104 | 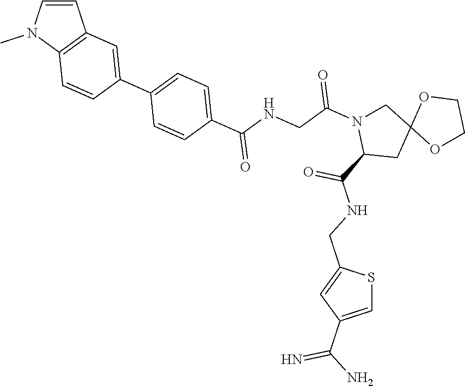 [SMILES: Cn1ccc2cc(-c3ccc(C(=O)NCC(=O)N4CC5(C[C@H]4C(=O)NCc4cc(C(=N)N)cs4)OCCO5)cc3)ccc21] | (S)-N-((4- carbamimidoylthiophen- 2-yl)methyl)-7-((4-(1- methyl-1H-indol-5- yl)benzoyl)glycyl)-1,4- dioxa-7- azaspiro[4.4]nonane-8- carboxamide | *** | 1.49 | 601 |
| 105 | 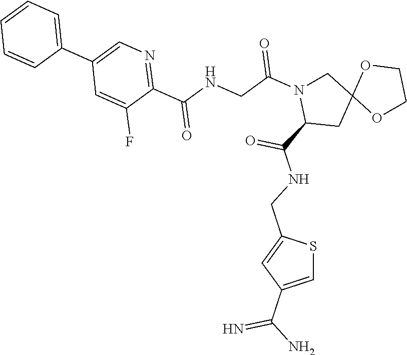 [SMILES: N=C(N)c1csc(CNC(=O)[C@@H]2CC3(CN2C(=O)CNC(=O)c2ncc(-c4ccccc4)cc2F)OCCO3)c1] | (S)-N-((4- carbamimidoylthiophen- 2-yl)methyl)-7-((3- fluoro-5- phenylpicolinoyl)glycyl)- 1,4-dioxa-7- azaspiro[4.4]nonane-8- carboxamide | *** | 1.26 | 567 |
| 106 | 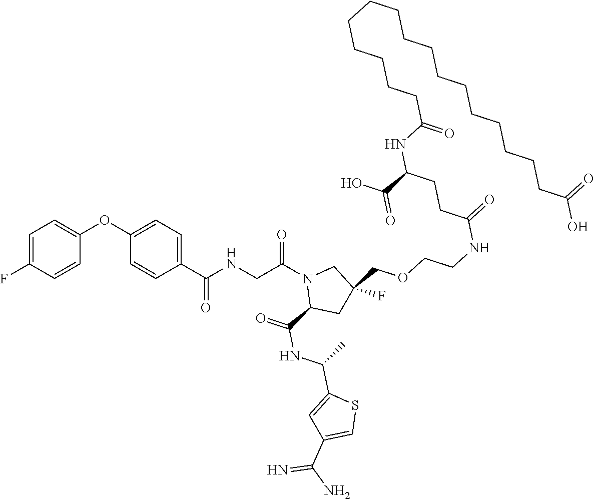 [SMILES: C[C@@H](NC(=O)[C@@H]1C[C@](F)(COCCNC(=O)CC[C@H](NC(=O)CCCCCCCCCCCCCCCCC(=O)O)C(=O)O)CN1C(=O)CNC(=O)c1ccc(Oc2ccc(F)cc2)cc1)c1cc(C(=N)N)cs1] | 18-(((S)-4-((2- (((3R,5S)-5-(((R)-1-(4- carbamimidoylthiophen- 2-yl)ethyl)carbamoyl)- 3-fluoro-1-((4-(4- fluorophenoxy)benzoyl) glycyl)pyrrolidin-3- yl)methoxy)ethyl)amino)- 1-carboxy-4- oxobutyl)amino)-18- oxooctadecanoic acid | *** | 2.28 | 1055 |
| 107 | 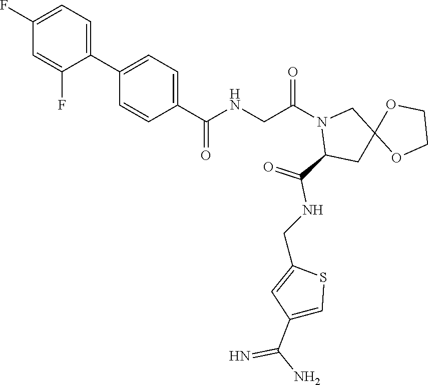 [SMILES: N=C(N)c1csc(CNC(=O)[C@@H]2CC3(CN2C(=O)CNC(=O)c2ccc(-c4ccc(F)cc4F)cc2)OCCO3)c1] | (S)-N-((4- carbamimidoylthiophen- 2-yl)methyl)-7-((2′,4′- difluoro-[1,1′-biphenyl]- 4-carbonyl)glycyl)-1,4- dioxa-7- azaspiro[4.4]nonane-8- carboxamide | *** | 1.46 | 584 |
| 108 | 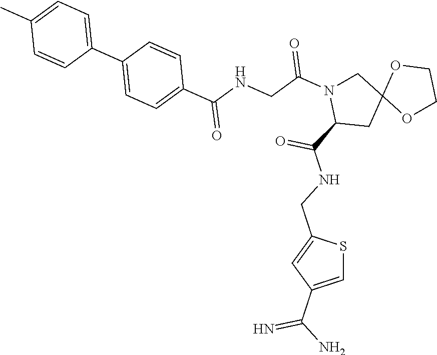 [SMILES: Cc1ccc(-c2ccc(C(=O)NCC(=O)N3CC4(C[C@H]3C(=O)NCc3cc(C(=N)N)cs3)OCCO4)cc2)cc1] | (S)-N-((4- carbamimidoylthiophen- 2-yl)methyl)-7-((4′- methyl-[1,1′-biphenyl]- 4-carbonyl)glycyl)-1,4- dioxa-7- azaspiro[4.4]nonane-8- carboxamide | *** | 1.58 | 584 |
| 109 | 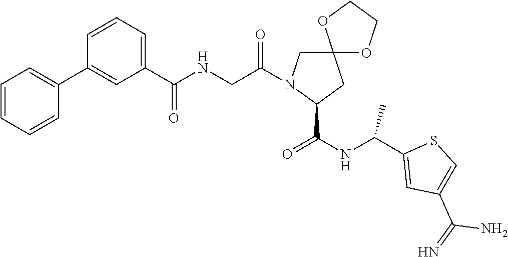 [SMILES: C[C@@H](NC(=O)[C@@H]1CC2(CN1C(=O)CNC(=O)c1cccc(-c3ccccc3)c1)OCCO2)c1cc(C(=N)N)cs1] | (S)-7-(([1,1′-biphenyl]- 3-carbonyl)glycyl)-N- ((R)-1-(4- carbamimidoylthiophen- 2-yl)ethyl)-1,4-dioxa-7- azaspiro[4.4]nonane-8- carboxamide | *** | 1.27 | 562 |
| 110 | 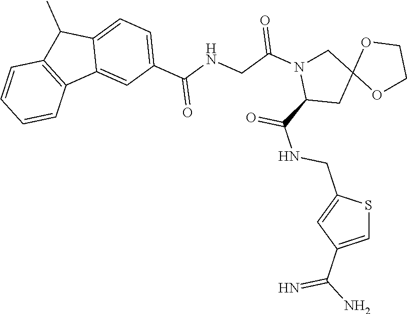 [SMILES: CC1c2ccccc2-c2cc(C(=O)NCC(=O)N3CC4(C[C@H]3C(=O)NCc3cc(C(=N)N)cs3)OCCO4)ccc21] | (8S)-N-((4- carbamimidoylthiophen- 2-yl)methyl)-7-((9- methyl-9H-fluorene-3- carbonyl)glycyl)-1,4- dioxa-7- azaspiro[4.4]nonane-8- carboxamide | *** | 1.36 | 574 |
| 111 | 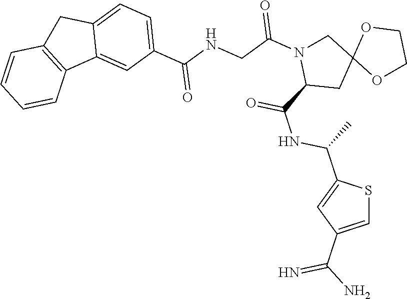 [SMILES: C[C@@H](NC(=O)[C@@H]1CC2(CN1C(=O)CNC(=O)c1ccc3c(c1)-c1ccccc1C3)OCCO2)c1cc(C(=N)N)cs1] | (S)-7-((9H-fluorene-3- carbonyl)glycyl)-N-((R)- 1-(4- carbamimidoylthiophen- 2-yl)ethyl)-1,4-dioxa-7- azaspiro[4.4]nonane-8- carboxamide | *** | 1.27 | 574 |
| 112 | 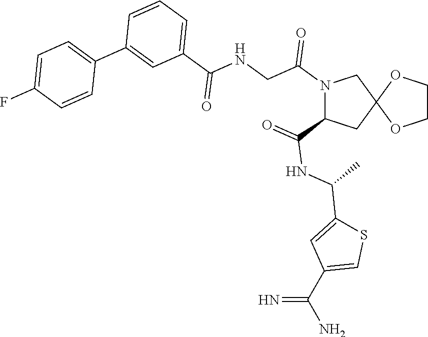 [SMILES: C[C@@H](NC(=O)[C@@H]1CC2(CN1C(=O)CNC(=O)c1cccc(-c3ccc(F)cc3)c1)OCCO2)c1cc(C(=N)N)cs1] | (S)-N-((R)-1-(4- carbamimidoylthiophen- 2-yl)ethyl)-7-((4′-fluoro- [1,1′-biphenyl]-3- carbonyl)glycyl)-1,4- dioxa-7- azaspiro[4.4]nonane-8- carboxamide | *** | 1.28 | 580 |
| 113 | 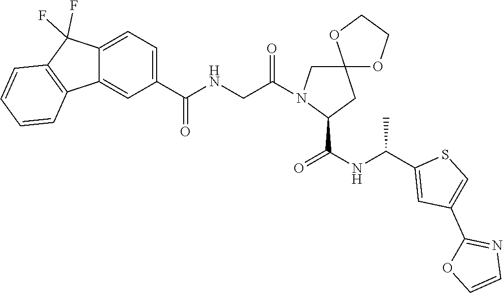 [SMILES: C[C@@H](NC(=O)[C@@H]1CC2(CN1C(=O)CNC(=O)c1ccc3c(c1)-c1ccccc1C3(F)F)OCCO2)c1cc(-c2ncco2)cs1] | (S)-7-((9,9-difluoro-9H- fluorene-3- carbonyl)glycyl)-N-((R)- 1-(4-(oxazol-2- yl)thiophen-2-yl)ethyl)- 1,4-dioxa-7- azaspiro[4.4]nonane-8- carboxamide | * | 2.32 | 635 |
| 114 | 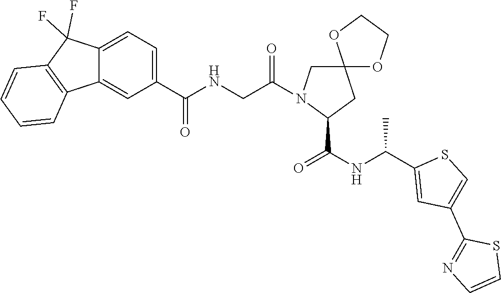 [SMILES: C[C@@H](NC(=O)[C@@H]1CC2(CN1C(=O)CNC(=O)c1ccc3c(c1)-c1ccccc1C3(F)F)OCCO2)c1cc(-c2nccs2)cs1] | (S)-7-((9,9-difluoro-9H- fluorene-3- carbonyl)glycyl)-N-((R)- 1-(4-(thiazol-2- yl)thiophen-2-yl)ethyl)- 1,4-dioxa-7- azaspiro[4.4]nonane-8- carboxamide | * | 2.42 | 651 |
| 115 | 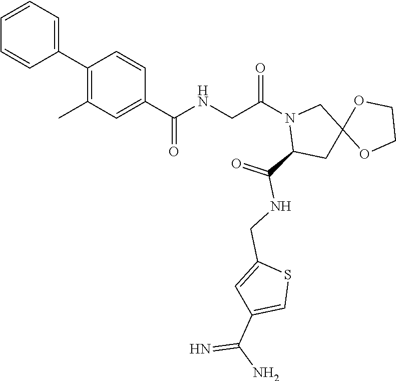 [SMILES: Cc1cc(C(=O)NCC(=O)N2CC3(C[C@H]2C(=O)NCc2cc(C(=N)N)cs2)OCCO3)ccc1-c1ccccc1] | (S)-N-((4- carbamimidoylthiophen- 2-yl)methyl)-7-((2- methyl-[1,1′-biphenyl]- 4-carbonyl)glycyl)-1,4- dioxa-7- azaspiro[4.4]nonane-8- carboxamide | *** | 1.41 | 562 |
| 116 | 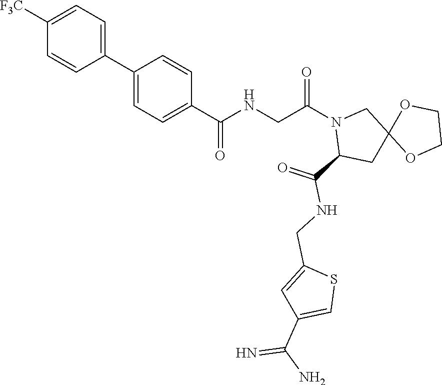 [SMILES: N=C(N)c1csc(CNC(=O)[C@@H]2CC3(CN2C(=O)CNC(=O)c2ccc(-c4ccc(C(F)(F)F)cc4)cc2)OCCO3)c1] | (S)-N-((4- carbamimidoylthiophen- 2-yl)methyl)-7-((4′- (trifluoromethyl)-[1,1′- biphenyl]-4- carbonyl)glycyl)-1,4- dioxa-7- azaspiro[4.4]nonane-8- carboxamide | *** | 1.65 | 616 |
| 117 | 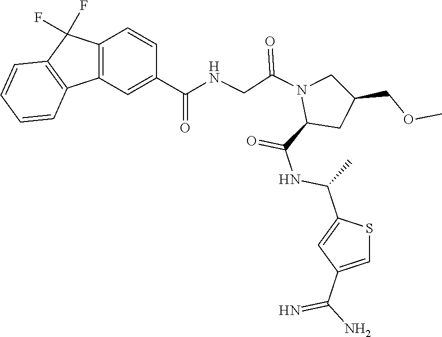 [SMILES: COC[C@H]1C[C@@H](C(=O)N[C@H](C)c2cc(C(=N)N)cs2)N(C(=O)CNC(=O)c2ccc3c(c2)-c2ccccc2C3(F)F)C1] | (2S,4S)-N-((R)-1-(4- carbamimidoylthiophen- 2-yl)ethyl)-1-((9,9- difluoro-9H-fluorene-3- carbonyl)glycyl)-4- (methoxymethyl) pyrrolidine-2- carboxamide | *** | 1.37 | 596 |
| 118 | 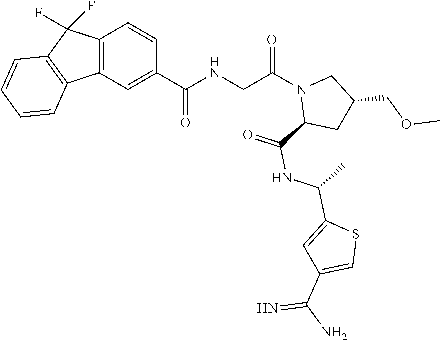 [SMILES: COC[C@@H]1C[C@@H](C(=O)N[C@H](C)c2cc(C(=N)N)cs2)N(C(=O)CNC(=O)c2ccc3c(c2)-c2ccccc2C3(F)F)C1] | (2S,4R)-N-((R)-1-(4- carbamimidoylthiophen- 2-yl)ethyl)-1-((9,9- difluoro-9H-fluorene-3- carbonyl)glycyl)-4- (methoxymethyl) pyrrolidine-2- carboxamide | *** | 1.39 | 596 |
| 119 | 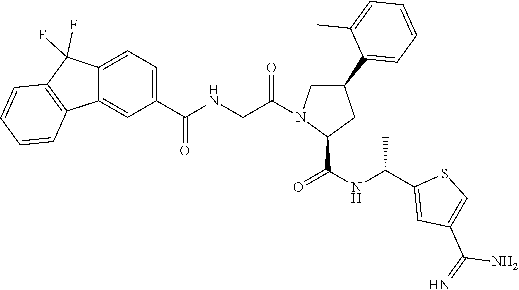 [SMILES: Cc1ccccc1[C@H]1C[C@@H](C(=O)N[C@H](C)c2cc(C(=N)N)cs2)N(C(=O)CNC(=O)c2ccc3c(c2)-c2ccccc2C3(F)F)C1] | (2S,4R)-N-((R)-1-(4- carbamimidoylthiophen- 2-yl)ethyl)-1-((9,9- difluoro-9H-fluorene-3- carbonyl)glycyl)-4-(o- tolyl)pyrrolidine-2- carboxamide | *** | 1.78 | 642 |
| 120 | 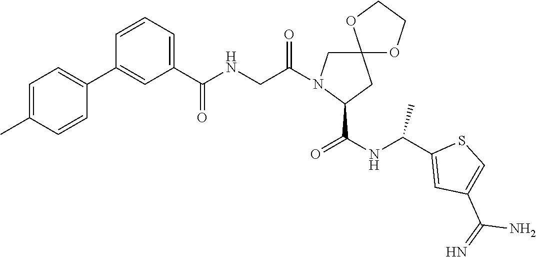 [SMILES: Cc1ccc(-c2cccc(C(=O)NCC(=O)N3CC4(C[C@H]3C(=O)N[C@H](C)c3cc(C(=N)N)cs3)OCCO4)c2)cc1] | (S)-N-((R)-1-(4- carbamimidoylthiophen- 2-yl)ethyl)-7-((4′- methyl-[1,1′-biphenyl]- 3-carbonyl)glycyl)-1,4- dioxa-7- azaspiro[4.4]nonane-8- carboxamide | *** | 1.37 | 576 |
| 121 | 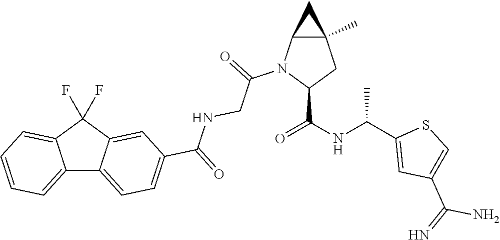 [SMILES: C[C@@H](NC(=O)[C@@H]1C[C@]2(C)C[C@@H]2N1C(=O)CNC(=O)c1ccc2c(c1)C(F)(F)c1ccccc1-2)c1cc(C(=N)N)cs1] | (1S,3S,5S)-N-((R)-1-(4- carbamimidoylthiophen- 2-yl)ethyl)-2-((9,9- difluoro-9H-fluorene-2- carbonyl)glycyl)-5- methyl-2- azabicyclo[3.1.0]hexane- 3-carboxamide | *** | 1.50 | 578 |
| 122 | 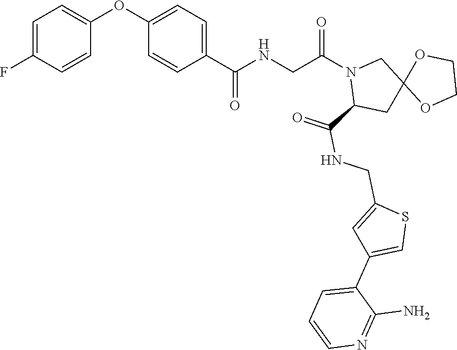 [SMILES: Nc1ncccc1-c1csc(CNC(=O)[C@@H]2CC3(CN2C(=O)CNC(=O)c2ccc(Oc4ccc(F)cc4)cc2)OCCO3)c1] | (S)-N-((4-(2- aminopyridin-3- yl)thiophen-2- yl)methyl)-7-((4-(4- fluorophenoxy)benzoyl) glycyl)-1,4-dioxa-7- azaspiro[4.4]nonane-8- carboxamide | * | 1.53 | 570 |
| 123 | 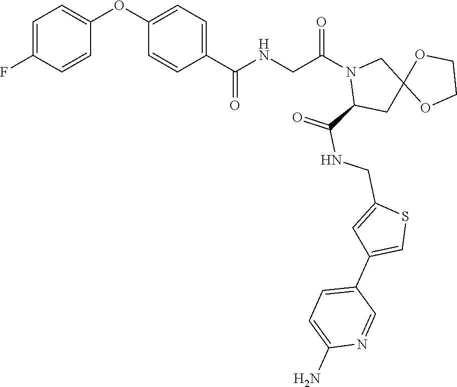 [SMILES: Nc1ccc(-c2csc(CNC(=O)[C@@H]3CC4(CN3C(=O)CNC(=O)c3ccc(Oc5ccc(F)cc5)cc3)OCCO4)c2)cn1] | (S)-N-((4-(6- aminopyridin-3- yl)thiophen-2- yl)methyl)-7-((4-(4- fluorophenoxy)benzoyl) glycyl)-1,4-dioxa-7- azaspiro[4.4]nonane-8- carboxamide | * | 1.52 | 632 |
| 124 | 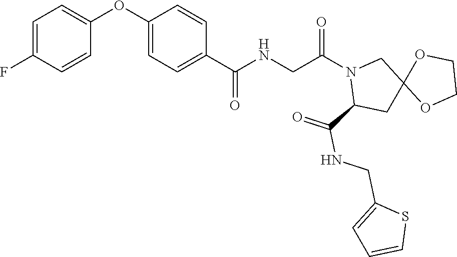 [SMILES: O=C(NCC(=O)N1CC2(C[C@H]1C(=O)NCc1cccs1)OCCO2)c1ccc(Oc2ccc(F)cc2)cc1] | (S)-7-((4-(4- fluorophenoxy)benzoyl) glycyl)-N-(thiophen-2- ylmethyl)-1,4-dioxa-7- azaspiro[4.4]nonane-8- carboxamide | * | 2.25 | 540 |
| 125 | 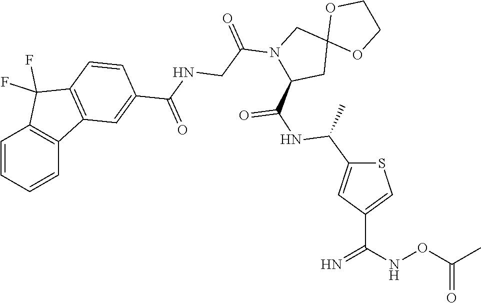 [SMILES: CC(=O)ONC(=N)c1csc([C@@H](C)NC(=O)[C@@H]2CC3(CN2C(=O)CNC(=O)c2ccc4c(c2)-c2ccccc2C4(F)F)OCCO3)c1] | (S)-N-((R)-1-(4-(N- acetoxycarbamimidoyl) thiophen-2-yl)ethyl)-7- ((9,9-difluoro-9H- fluorene-3- carbonyl)glycyl)-1,4- dioxa-7- azaspiro[4.4]nonane-8- carboxamide | *** | 2.01 | 668 |
| 126 | 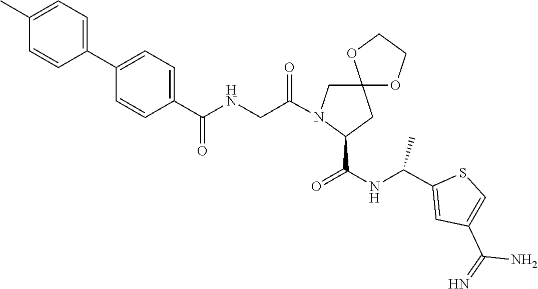 [SMILES: Cc1ccc(-c2ccc(C(=O)NCC(=O)N3CC4(C[C@H]3C(=O)N[C@H](C)c3cc(C(=N)N)cs3)OCCO4)cc2)cc1] | (S)-N-((R)-1-(4- carbamimidoylthiophen- 2-yl)ethyl)-7-((4′- methyl-[1,1′-biphenyl]- 4-carbonyl)glycyl)-1,4- dioxa-7- azaspiro[4.4]nonane-8- carboxamide | *** | 1.33 | 576 |
| 127 | 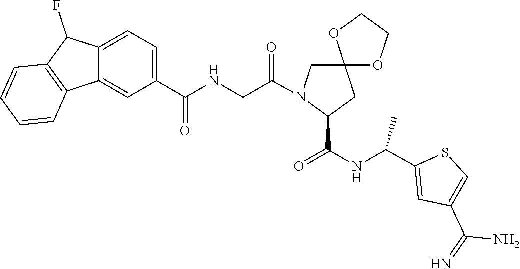 [SMILES: C[C@@H](NC(=O)[C@@H]1CC2(CN1C(=O)CNC(=O)c1ccc3c(c1)-c1ccccc1C3F)OCCO2)c1cc(C(=N)N)cs1] | (8S)-N-((R)-1-(4- carbamimidoylthiophen- 2-yl)ethyl)-7-((9-fluoro- 9H-fluorene-3- carbonyl)glycyl)-1,4- dioxa-7- azaspiro[4.4]nonane-8- carboxamide | *** | 1.16 | 592 |
| 128 | 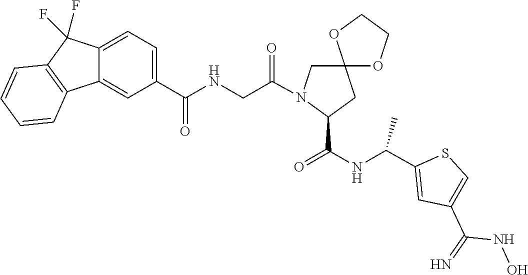 [SMILES: C[C@@H](NC(=O)[C@@H]1CC2(CN1C(=O)CNC(=O)c1ccc3c(c1)-c1ccccc1C3(F)F)OCCO2)c1cc(C(=N)NO)cs1] | (S)-7-((9,9-difluoro-9H- fluorene-3- carbonyl)glycyl)-N-((R)- 1-(4-(N- hydroxycarbamimidoyl) thiophen-2-yl)ethyl)-1,4- dioxa-7- azaspiro[4.4]nonane-8- carboxamide | *** | 1.55 | 626 |
| 129 | 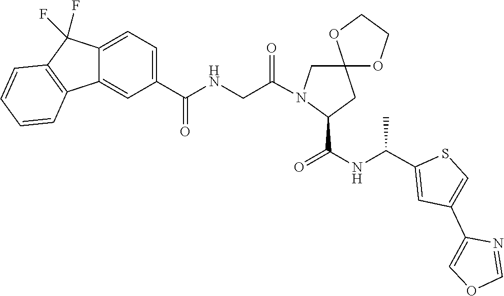 [SMILES: C[C@@H](NC(=O)[C@@H]1CC2(CN1C(=O)CNC(=O)c1ccc3c(c1)-c1ccccc1C3(F)F)OCCO2)c1cc(-c2cocn2)cs1] | (S)-7-((9,9-difluoro-9H- fluorene-3- carbonyl)glycyl)-N-((R)- 1-(4-(oxazol-4- yl)thiophen-2-yl)ethyl)- 1,4-dioxa-7- azaspiro[4.4]nonane-8- carboxamide | * | 2.26 | 635 |
| 130 | 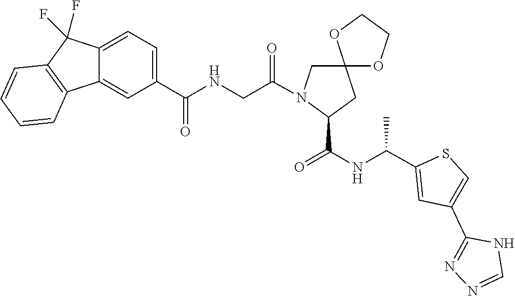 [SMILES: C[C@@H](NC(=O)[C@@H]1CC2(CN1C(=O)CNC(=O)c1ccc3c(c1)-c1ccccc1C3(F)F)OCCO2)c1cc(-c2nnc[nH]2)cs1] | (S)-N-((R)-1-(4-(4H- 1,2,4-triazol-3- yl)thiophen-2-yl)ethyl)- 7-((9,9-difluoro-9H- fluorene-3- carbonyl)glycyl)-1,4- dioxa-7- azaspiro[4.4]nonane-8- carboxamide | ** | 1.89 | 635 |
| 131 | 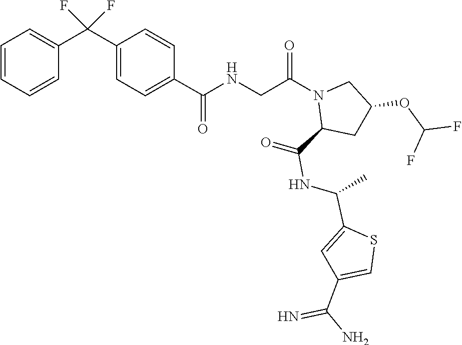 [SMILES: C[C@@H](NC(=O)[C@@H]1C[C@@H](OC(F)F)CN1C(=O)CNC(=O)c1ccc(C(F)(F)c2ccccc2)cc1)c1cc(C(=N)N)cs1] | (2S,4R)-N-((R)-1-(4- carbamimidoylthiophen- 2-yl)ethyl)-1-((4- (difluoro(phenyl)methyl) benzoyl)glycyl)-4- (difluoromethoxy) pyrrolidine-2- carboxamide | *** | 1.44 | 620 |
| 132 | 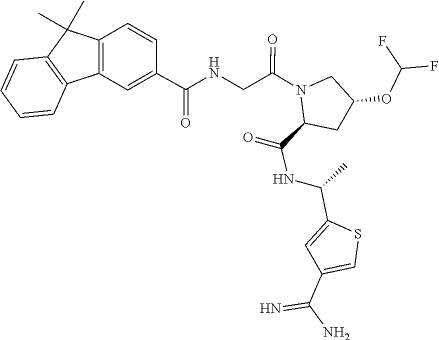 [SMILES: C[C@@H](NC(=O)[C@@H]1C[C@@H](OC(F)F)CN1C(=O)CNC(=O)c1ccc2c(c1)-c1ccccc1C2(C)C)c1cc(C(=N)N)cs1] | (2S,4R)-N-((R)-1-(4- carbamimidoylthiophen- 2-yl)ethyl)-4- (difluoromethoxy)-1- ((9,9-dimethyl-9H- fluorene-3- carbonyl)glycyl) pyrrolidine-2- carboxamide | *** | 1.54 | 610 |
| 133 | 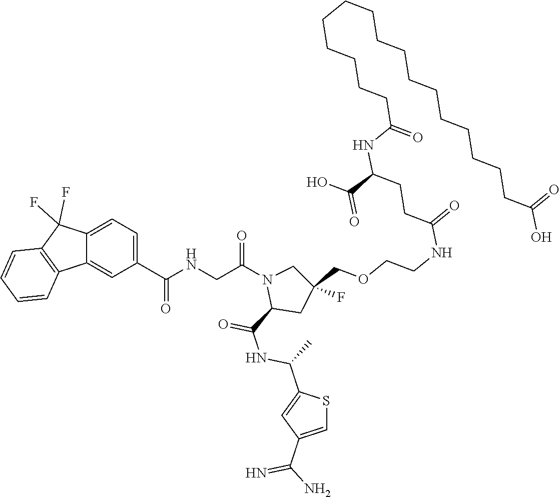 [SMILES: C[C@@H](NC(=O)[C@@H]1C[C@](F)(COCCNC(=O)CC[C@H](NC(=O)CCCCCCCCCCCCCCCCC(=O)O)C(=O)O)CN1C(=O)CNC(=O)c1ccc2c(c1)-c1ccccc1C2(F)F)c1cc(C(=N)N)cs1] | 18-(((S)-4-((2- (((3R,5S)-5-(((R)-1-(4- carbamimidoylthiophen- 2-yl)ethyl)carbamoyl)- 1-((9,9-difluoro-9H- fluorene-3- carbonyl)glycyl)-3- fluoropyrrolidin-3- yl)methoxy)ethyl)amino)- 1-carboxy-4- oxobutyl)amino)-18- oxooctadecanoic acid | *** | 2.24 | 1068 |
| 134 | 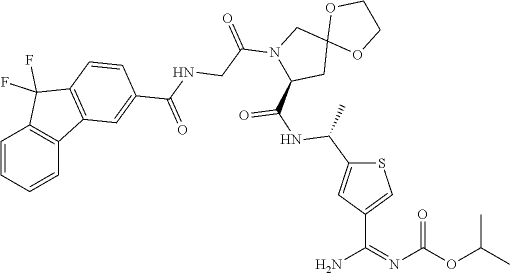 [SMILES: CC(C)OC(=O)/N=C(/N)c1csc([C@@H](C)NC(=O)[C@@H]2CC3(CN2C(=O)CNC(=O)c2ccc4c(c2)-c2ccccc2C4(F)F)OCCO3)c1] | (E)-isopropyl (amino(5- ((R)-1-((S)-7-((9,9- difluoro-9H-fluorene-3- carbonyl)glycyl)-1,4- dioxa-7- azaspiro[4.4]nonane-8- carboxamido)ethyl) thiophen-3- yl)methylene)carbamate | *** | 1.61 | 696 |
| 135 | 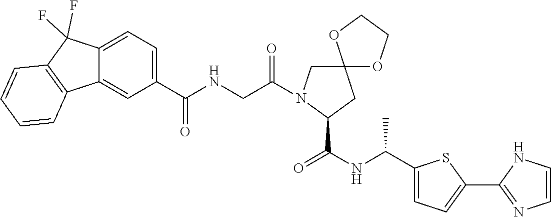 [SMILES: C[C@@H](NC(=O)[C@@H]1CC2(CN1C(=O)CNC(=O)c1ccc3c(c1)-c1ccccc1C3(F)F)OCCO2)c1ccc(-c2ncc[nH]2)s1] | (S)-N-((R)-1-(5-(1H- imidazol-2-yl)thiophen- 2-yl)ethyl)-7-((9,9- difluoro-9H-fluorene-3- carbonyl)glycyl)-1,4- dioxa-7- azaspiro[4.4]nonane-8- carboxamide | * | 1.35 | 634 |
| 136 | 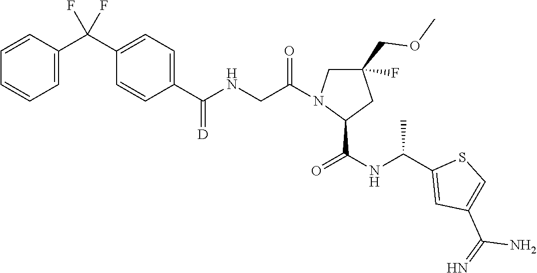 | (2S,4R)-N-((R)-1-(4- carbamimidoylthiophen- 2-yl)ethyl)-1-((4- (difluoro(phenyl)methyl) benzoyl)glycyl)-4- fluoro-4- (methoxymethyl) pyrrolidine-2- carboxamide | *** | 1.37 | 616 |
| 137 | 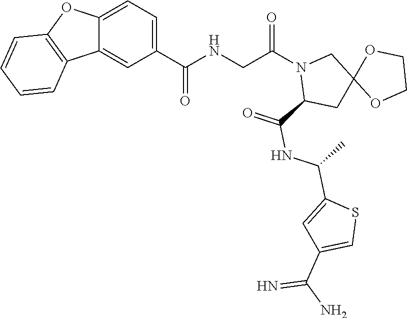 [SMILES: C[C@@H](NC(=O)[C@@H]1CC2(CN1C(=O)CNC(=O)c1ccc3oc4ccccc4c3c1)OCCO2)c1cc(C(=N)N)cs1] | (S)-N-((R)-1-(4- carbamimidoylthiophen- 2-yl)ethyl)-7- ((dibenzo[b,d]furan-2- carbonyl)glycyl)-1,4- dioxa-7- azaspiro[4.4]nonane-8- carboxamide | *** | 1.16 | 576 |
| 138 | 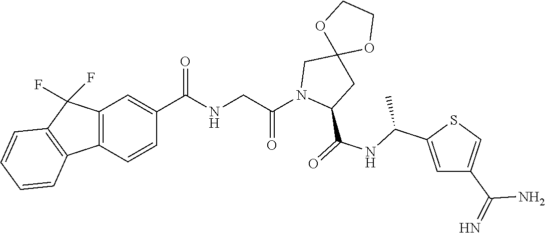 [SMILES: C[C@@H](NC(=O)[C@@H]1CC2(CN1C(=O)CNC(=O)c1ccc3c(c1)C(F)(F)c1ccccc1-3)OCCO2)c1cc(C(=N)N)cs1] | (S)-N-((R)-1-(4- carbamimidoylthiophen- 2-yl)ethyl)-7-((9,9- difluoro-9H-fluorene-2- carbonyl)glycyl)-1,4- dioxa-7- azaspiro[4.4]nonane-8- carboxamide | *** | 1.28 | 610 |
| 139 | 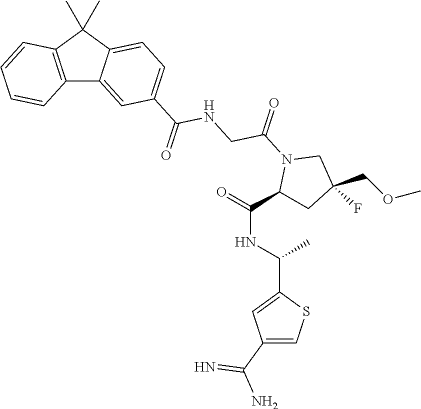 [SMILES: COC[C@@]1(F)C[C@@H](C(=O)N[C@H](C)c2cc(C(=N)N)cs2)N(C(=O)CNC(=O)c2ccc3c(c2)-c2ccccc2C3(C)C)C1] | (2S,4R)-N-((R)-1-(4- carbamimidoylthiophen- 2-yl)ethyl)-1-((9,9- dimethyl-9H-fluorene-3- carbonyl)glycyl)-4- fluoro-4- (methoxymethyl) pyrrolidine-2- carboxamide | *** | 1.43 | 606 |
| 140 | 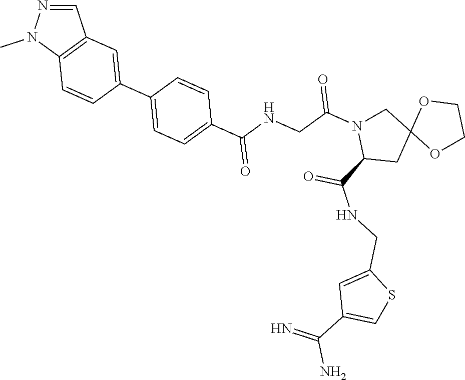 [SMILES: Cn1ncc2cc(-c3ccc(C(=O)NCC(=O)N4CC5(C[C@H]4C(=O)NCc4cc(C(=N)N)cs4)OCCO5)cc3)ccc21] | (S)-N-((4- carbamimidoylthiophen- 2-yl)methyl)-7-((4-(1- methyl-1H-indazol-5- yl)benzoyl)glycyl)-1,4- dioxa-7- azaspiro[4.4]nonane-8- carboxamide | *** | 1.14 | 602 |
| 141 | 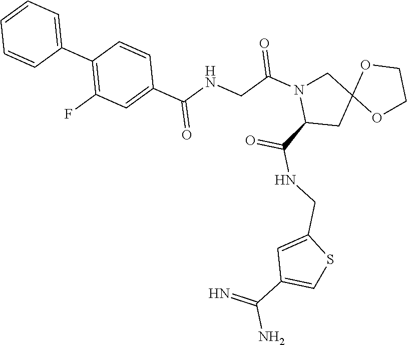 [SMILES: N=C(N)c1csc(CNC(=O)[C@@H]2CC3(CN2C(=O)CNC(=O)c2ccc(-c4ccccc4)c(F)c2)OCCO3)c1] | (S)-N-((4- carbamimidoylthiophen- 2-yl)methyl)-7-((2- fluoro-[1,1′-biphenyl]-4- carbonyl)glycyl)-1,4- dioxa-7- azaspiro[4.4]nonane-8- carboxamide | *** | 1.32 | 566 |
| 142 | 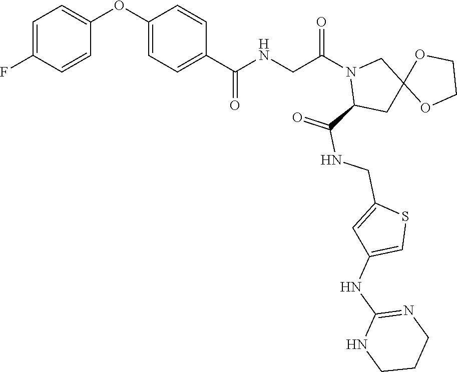 [SMILES: O=C(NCC(=O)N1CC2(C[C@H]1C(=O)NCc1cc(NC3=NCCCN3)cs1)OCCO2)c1ccc(Oc2ccc(F)cc2)cc1] | (S)-7-((4-(4- fluorophenoxy)benzoyl) glycyl)-N-((4-((1,4,5,6- tetrahydropyrimidin-2- yl)amino)thiophen-2- yl)methyl)-1,4-dioxa-7- azaspiro[4.4]nonane-8- carboxamide | * | 1.47 | 637 |
| 143 | 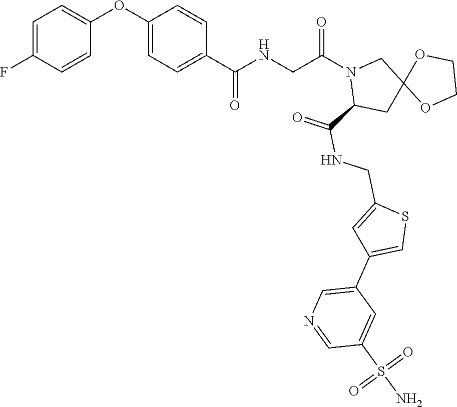 [SMILES: NS(=O)(=O)c1cncc(-c2csc(CNC(=O)[C@@H]3CC4(CN3C(=O)CNC(=O)c3ccc(Oc5ccc(F)cc5)cc3)OCCO4)c2)c1] | (S)-7-((4-(4- fluorophenoxy)benzoyl) glycyl)-N-((4-(5- sulfamoylpyridin-3- yl)thiophen-2- yl)methyl)-1,4-dioxa-7- azaspiro[4.4]nonane-8- carboxamide | * | 1.99 | 696 |
| 144 | 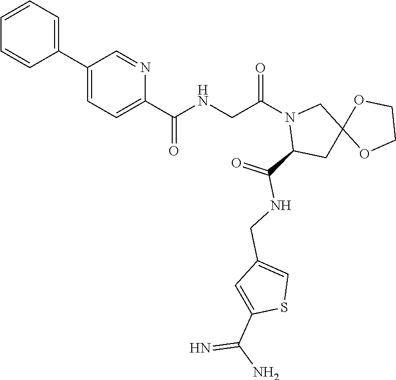 [SMILES: N=C(N)c1cc(CNC(=O)[C@@H]2CC3(CN2C(=O)CNC(=O)c2ccc(-c4ccccc4)cn2)OCCO3)cs1] | (S)-N-((5- carbamimidoylthiophen- 3-yl)methyl)-7-((5- phenylpicolinoyl)glycyl)- 1,4-dioxa-7- azaspiro[4.4]nonane-8- carboxamide | *** | 1.28 | 548 |
| 145 | 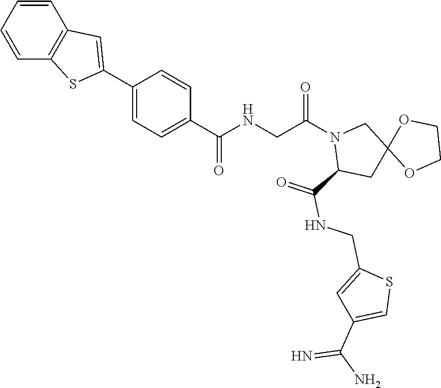 [SMILES: N=C(N)c1csc(CNC(=O)[C@@H]2CC3(CN2C(=O)CNC(=O)c2ccc(-c4cc5ccccc5s4)cc2)OCCO3)c1] | (S)-7-((4- (benzo[b]thiophen-2- yl)benzoyl)glycyl)-N- ((4- carbamimidoylthiophen- 2-yl)methyl)-1,4-dioxa- 7-azaspiro[4.4]nonane- 8-carboxamide | *** | 1.62 | 604 |
| 146 | 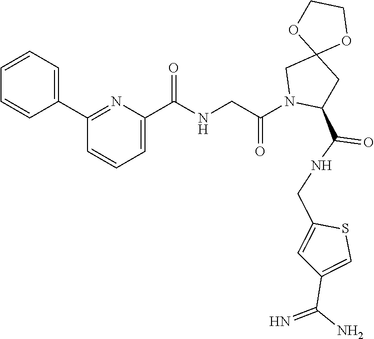 [SMILES: N=C(N)c1csc(CNC(=O)[C@@H]2CC3(CN2C(=O)CNC(=O)c2cccc(-c4ccccc4)n2)OCCO3)c1] | (S)-N-((4- carbamimidoylthiophen- 2-yl)methyl)-7-((6- phenylpicolinoyl)glycyl)- 1,4-dioxa-7- azaspiro[4.4]nonane-8- carboxamide | *** | 1.20 | 549 |
| 147 | 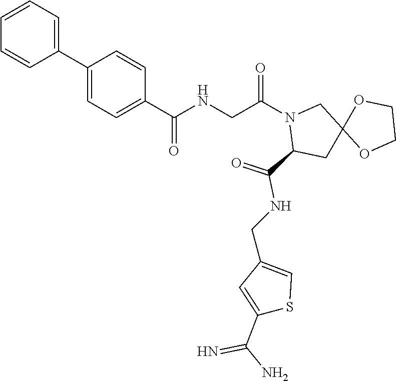 [SMILES: N=C(N)c1cc(CNC(=O)[C@@H]2CC3(CN2C(=O)CNC(=O)c2ccc(-c4ccccc4)cc2)OCCO3)cs1] | (S)-7-(([1,1′-biphenyl]- 4-carbonyl)glycyl)-N- ((5- carbamimidoylthiophen- 3-yl)methyl)-1,4-dioxa- 7-azaspiro[4.4]nonane- 8-carboxamide | *** | 1.28 | 548 |
| 148 | 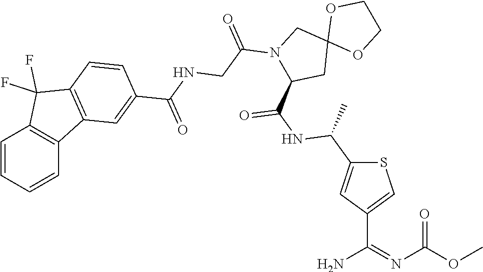 [SMILES: COC(=O)/N=C(/N)c1csc([C@@H](C)NC(=O)[C@@H]2CC3(CN2C(=O)CNC(=O)c2ccc4c(c2)-c2ccccc2C4(F)F)OCCO3)c1] | (E)-methyl (amino(5- ((R)-1-((S)-7-((9,9- difluoro-9H-fluorene-3- carbonyl)glycyl)-1,4- dioxa-7- azaspiro[4.4]nonane-8- carboxamido)ethyl) thiophen-3- yl)methylene)carbamate | *** | 1.16 | 668 |
| 149 | 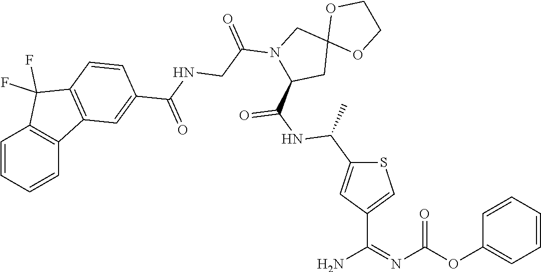 [SMILES: C[C@@H](NC(=O)[C@@H]1CC2(CN1C(=O)CNC(=O)c1ccc3c(c1)-c1ccccc1C3(F)F)OCCO2)c1cc(/C(N)=N\C(=O)Oc2ccccc2)cs1] | (E)-phenyl (amino(5- ((R)-1-((S)-7-((9,9- difluoro-9H-fluorene-3- carbonyl)glycyl)-1,4- dioxa-7- azaspiro[4.4]nonane-8- carboxamido)ethyl) thiophen-3- yl)methylene)carbamate | *** | 2.17 | 730 |
| 150 | 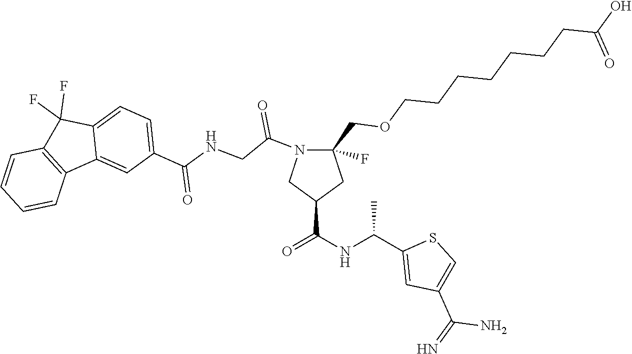 [SMILES: C[C@@H](NC(=O)[C@H]1CN(C(=O)CNC(=O)c2ccc3c(c2)-c2ccccc2C3(F)F)[C@@](F)(COCCCCCCCC(=O)O)C1)c1cc(C(=N)N)cs1] | 8-(((3R,5S)-5-(((R)-1- (4- carbamimidoylthiophen- 2-yl)ethyl)carbamoyl)- 1-((9,9-difluoro-9H- fluorene-3- carbonyl)glycyl)-3- fluoropyrrolidin-3- yl)methoxy)octanoic acid | *** | 1.66 | 742 |
| 151 | 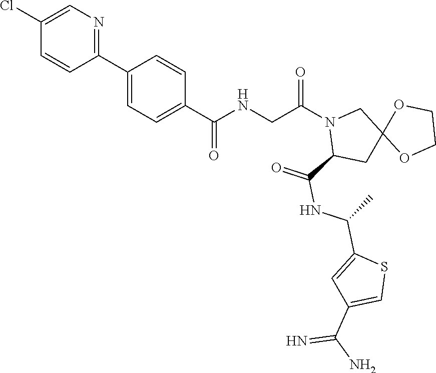 [SMILES: C[C@@H](NC(=O)[C@@H]1CC2(CN1C(=O)CNC(=O)c1ccc(-c3ccc(Cl)cn3)cc1)OCCO2)c1cc(C(=N)N)cs1] | (S)-N-((R)-1-(4- carbamimidoylthiophen- 2-yl)ethyl)-7-((4-(5- chloropyridin-2- yl)benzoyl)glycyl)-1,4- dioxa-7- azaspiro[4.4]nonane-8- carboxamide | *** | 1.12 | 598 |
| 152 | 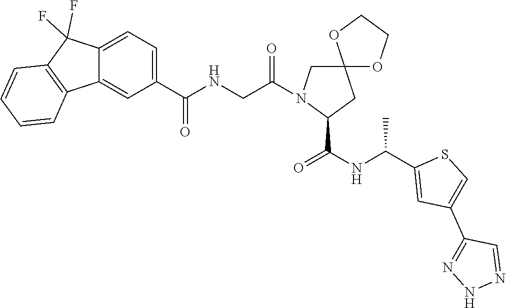 [SMILES: C[C@@H](NC(=O)[C@@H]1CC2(CN1C(=O)CNC(=O)c1ccc3c(c1)-c1ccccc1C3(F)F)OCCO2)c1cc(-c2cn[nH]n2)cs1] | (S)-N-((R)-1-(4-(2H- 1,2,3-triazol-4- yl)thiophen-2-yl)ethyl)- 7-((9,9-difluoro-9H- fluorene-3- carbonyl)glycyl)-1,4- dioxa-7- azaspiro[4.4]nonane-8- carboxamide | * | 2.01 | 635 |
| 153 | 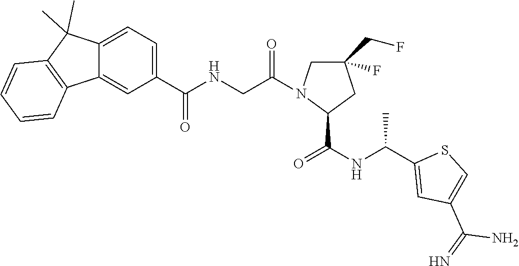 [SMILES: C[C@@H](NC(=O)[C@@H]1C[C@](F)(CF)CN1C(=O)CNC(=O)c1ccc2c(c1)-c1ccccc1C2(C)C)c1cc(C(=N)N)cs1] | (2S,4R)-N-((R)-1-(4- carbamimidoylthiophen- 2-yl)ethyl)-1-((9,9- dimethyl-9H-fluorene-3- carbonyl)glycyl)-4- fluoro-4- (fluoromethyl)pyrrolidine- 2-carboxamide | *** | 1.52 | 594 |
| 154 | 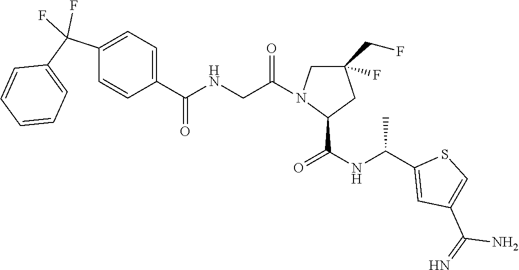 [SMILES: C[C@@H](NC(=O)[C@@H]1C[C@](F)(CF)CN1C(=O)CNC(=O)c1ccc(C(F)(F)c2ccccc2)cc1)c1cc(C(=N)N)cs1] | (2S,4R)-N-((R)-1-(4- carbamimidoylthiophen- 2-yl)ethyl)-1-((4- (difluoro(phenyl)methyl) benzoyl)glycyl)-4- fluoro-4- (fluoromethyl)pyrrolidine- 2-carboxamide | *** | 1.19 | 604 |
| 155 | 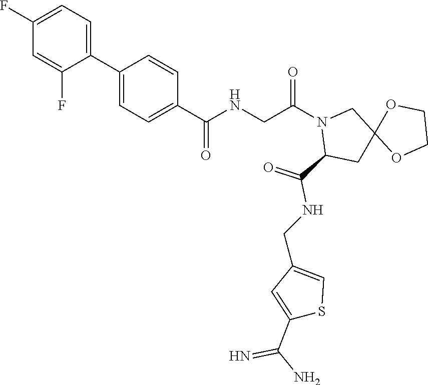 [SMILES: N=C(N)c1cc(CNC(=O)[C@@H]2CC3(CN2C(=O)CNC(=O)c2ccc(-c4ccc(F)cc4F)cc2)OCCO3)cs1] | (S)-N-((5- carbamimidoylthiophen- 3-yl)methyl)-7-((2′,4′- difluoro-[1,1′-biphenyl]- 4-carbonyl)glycyl)-1,4- dioxa-7- azaspiro[4.4]nonane-8- carboxamide | *** | 1.36 | 584 |
| 156 | 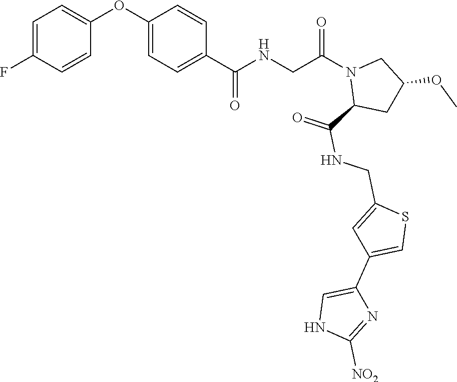 [SMILES: CO[C@@H]1C[C@@H](C(=O)NCc2cc(-c3c[nH]c([N+](=O)[O-])n3)cs2)N(C(=O)CNC(=O)c2ccc(Oc3ccc(F)cc3)cc2)C1] | (2S,4R)-1-((4-(4- fluorophenoxy)benzoyl) glycyl)-4-methoxy-N- ((4-(2-nitro-1H- imidazol-4-yl)thiophen- 2-yl)methyl)pyrrolidine- 2-carboxamide | * | 2.00 | 623 |
| 157 | 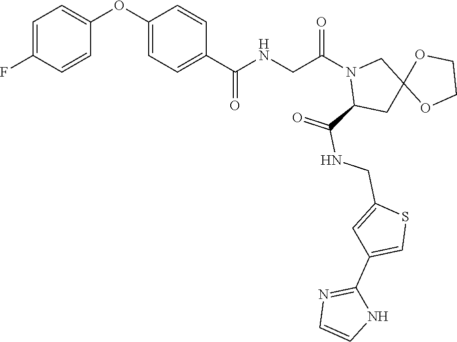 [SMILES: O=C(NCC(=O)N1CC2(C[C@H]1C(=O)NCc1cc(-c3ncc[nH]3)cs1)OCCO2)c1ccc(Oc2ccc(F)cc2)cc1] | (S)-N-((4-(1H-imidazol- 2-yl)thiophen-2- yl)methyl)-7-((4-(4- fluorophenoxy)benzoyl) glycyl)-1,4-dioxa-7- azaspiro[4.4]nonane-8- carboxamide | * | 1.36 | 606 |
| 158 | 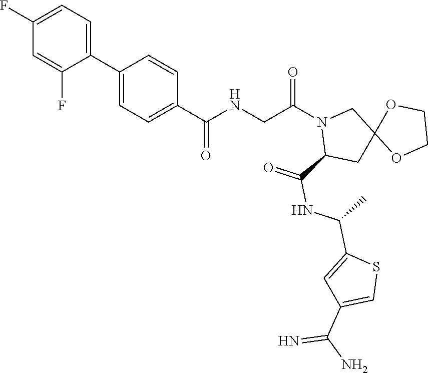 [SMILES: C[C@@H](NC(=O)[C@@H]1CC2(CN1C(=O)CNC(=O)c1ccc(-c3ccc(F)cc3F)cc1)OCCO2)c1cc(C(=N)N)cs1] | (S)-N-((R)-1-(4- carbamimidoylthiophen- 2-yl)ethyl)-7-((2′,4′- difluoro-[1,1′-biphenyl]- 4-carbonyl)glycyl)-1,4- dioxa-7- azaspiro[4.4]nonane-8- carboxamide | *** | 1.27 | 598 |
| 159 | 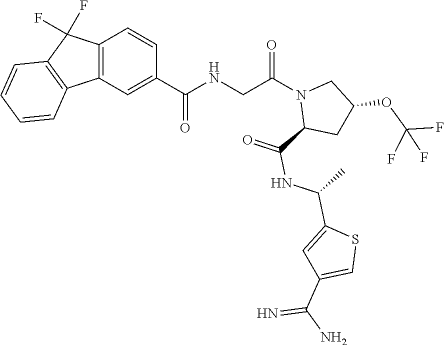 [SMILES: C[C@@H](NC(=O)[C@@H]1C[C@@H](OC(F)(F)F)CN1C(=O)CNC(=O)c1ccc2c(c1)-c1ccccc1C2(F)F)c1cc(C(=N)N)cs1] | (2S,4R)-N-((R)-1-(4- carbamimidoylthiophen- 2-yl)ethyl)-1-((9,9- difluoro-9H-fluorene-3- carbonyl)glycyl)-4- (trifluoromethoxy) pyrrolidine-2- carboxamide | *** | 1.61 | 636 |
| 160 | 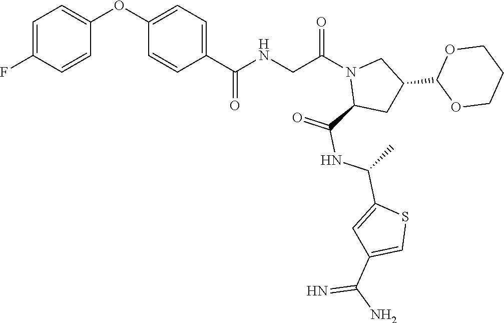 [SMILES: C[C@@H](NC(=O)[C@@H]1C[C@@H](C2OCCCO2)CN1C(=O)CNC(=O)c1ccc(Oc2ccc(F)cc2)cc1)c1cc(C(=N)N)cs1] | (2S,4R)-N-((R)-1-(4- carbamimidoylthiophen- 2-yl)ethyl)-4-(1,3- dioxan-2-yl)-1-((4-(4- fluorophenoxy)benzoyl) glycyl)pyrrolidine-2- carboxamide | *** | 1.35 | 624 |
| 161 | 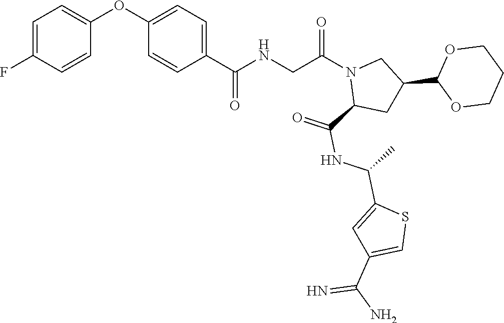 [SMILES: C[C@@H](NC(=O)[C@@H]1C[C@H](C2OCCCO2)CN1C(=O)CNC(=O)c1ccc(Oc2ccc(F)cc2)cc1)c1cc(C(=N)N)cs1] | (2S,4S)-N-((R)-1-(4- carbamimidoylthiophen- 2-yl)ethyl)-4-(1,3- dioxan-2-yl)-1-((4-(4- fluorophenoxy)benzoyl) glycyl)pyrrolidine-2- carboxamide | *** | 1.31 | 624 |
| 162 | 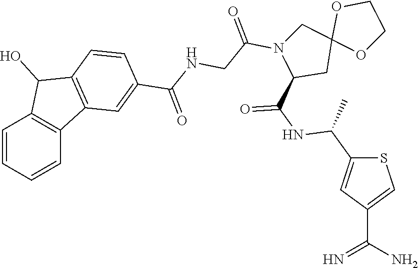 [SMILES: C[C@@H](NC(=O)[C@@H]1CC2(CN1C(=O)CNC(=O)c1ccc3c(c1)-c1ccccc1C3O)OCCO2)c1cc(C(=N)N)cs1] | (8S)-N-((R)-1-(4- carbamimidoylthiophen- 2-yl)ethyl)-7-((9- hydroxy-9H-fluorene-3- carbonyl)glycyl)-1,4- dioxa-7- azaspiro[4.4]nonane-8- carboxamide | *** | 1.09 | 590 |
| 163 | 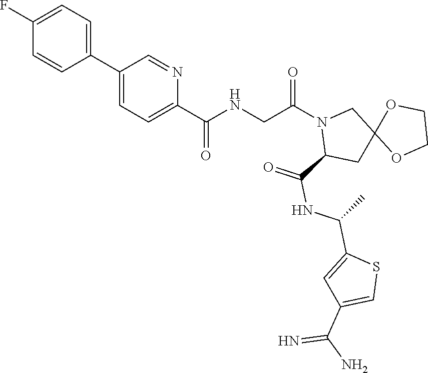 [SMILES: C[C@@H](NC(=O)[C@@H]1CC2(CN1C(=O)CNC(=O)c1ccc(-c3ccc(F)cc3)cn1)OCCO2)c1cc(C(=N)N)cs1] | (S)-N-((R)-1-(4- carbamimidoylthiophen- 2-yl)ethyl)-7-((5-(4- fluorophenyl)picolinoyl) glycyl)-1,4-dioxa-7- azaspiro[4.4]nonane-8- carboxamide | *** | 1.19 | 581 |
| 164 | 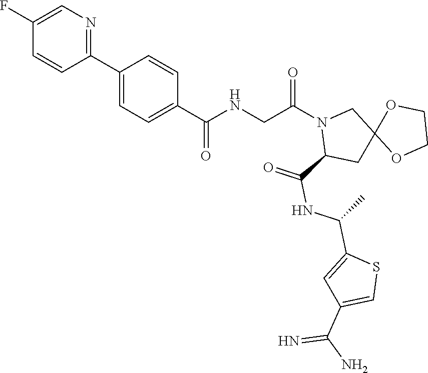 [SMILES: C[C@@H](NC(=O)[C@@H]1CC2(CN1C(=O)CNC(=O)c1ccc(-c3ccc(F)cn3)cc1)OCCO2)c1cc(C(=N)N)cs1] | (S)-N-((R)-1-(4- carbamimidoylthiophen- 2-yl)ethyl)-7-((4-(5- fluoropyridin-2- yl)benzoyl)glycyl)-1,4- dioxa-7- azaspiro[4.4]nonane-8- carboxamide | *** | 0.93 | 581 |
| 165 | 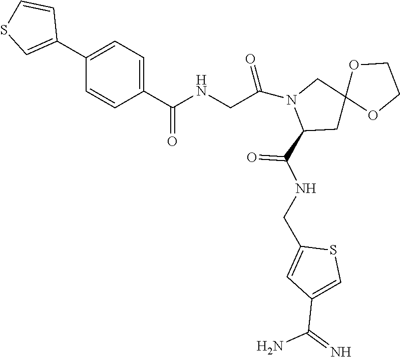 [SMILES: N=C(N)c1csc(CNC(=O)[C@@H]2CC3(CN2C(=O)CNC(=O)c2ccc(-c4ccsc4)cc2)OCCO3)c1] | (S)-N-((4- carbamimidoylthiophen- 2-yl)methyl)-7-((4- (thiophen-3- yl)benzoyl)glycyl)-1,4- dioxa-7- azaspiro[4.4]nonane-8- carboxamide | *** | 1.18 | 554 |
| 166 | 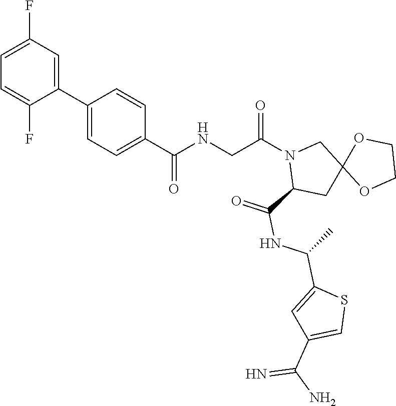 [SMILES: C[C@@H](NC(=O)[C@@H]1CC2(CN1C(=O)CNC(=O)c1ccc(-c3cc(F)ccc3F)cc1)OCCO2)c1cc(C(=N)N)cs1] | (S)-N-((R)-1-(4- carbamimidoylthiophen- 2-yl)ethyl)-7-((2′,5′- difluoro-[1,1′-biphenyl]- 4-carbonyl)glycyl)-1,4- dioxa-7- azaspiro[4.4]nonane-8- carboxamide | *** | 1.30 | 598 |
| 167 | 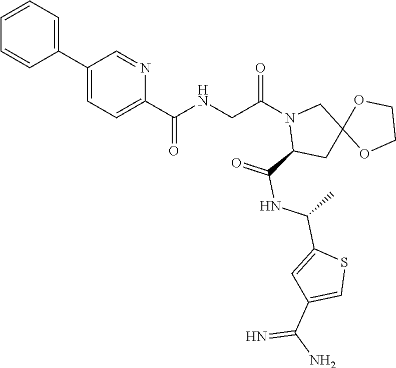 [SMILES: C[C@@H](NC(=O)[C@@H]1CC2(CN1C(=O)CNC(=O)c1ccc(-c3ccccc3)cn1)OCCO2)c1cc(C(=N)N)cs1] | (S)-N-((R)-1-(4- carbamimidoylthiophen- 2-yl)ethyl)-7-((5- phenylpicolinoyl)glycyl)- 1,4-dioxa-7- azaspiro[4.4]nonane-8- carboxamide | *** | 1.20 | 563 |
| 168 | 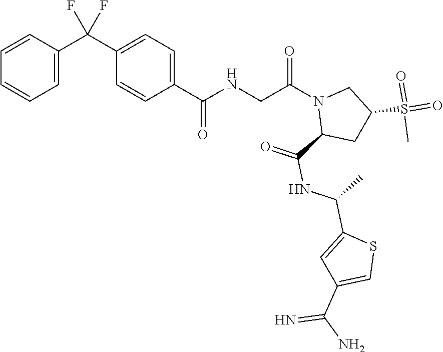 [SMILES: C[C@@H](NC(=O)[C@@H]1C[C@@H](S(C)(=O)=O)CN1C(=O)CNC(=O)c1ccc(C(F)(F)c2ccccc2)cc1)c1cc(C(=N)N)cs1] | (2S,4R)-N-((R)-1-(4- carbamimidoylthiophen- 2-yl)ethyl)-1-((4- (difluoro(phenyl)methyl) benzoyl)glycyl)-4- (methylsulfonyl) pyrrolidine-2- carboxamide | *** | 1.36 ( | 632 |
| 169 | 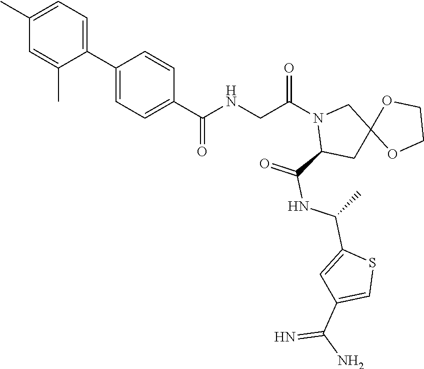 [SMILES: Cc1ccc(-c2ccc(C(=O)NCC(=O)N3CC4(C[C@H]3C(=O)N[C@H](C)c3cc(C(=N)N)cs3)OCCO4)cc2)c(C)c1] | (S)-N-((R)-1-(4- carbamimidoylthiophen- 2-yl)ethyl)-7-((2′,4′- dimethyl-[1,1′- biphenyl]-4- carbonyl)glycyl)-1,4- dioxa-7- azaspiro[4.4]nonane-8- carboxamide | *** | 1.54 | 590 |
| 170 | 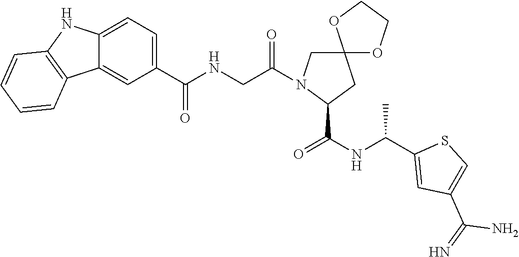 [SMILES: C[C@@H](NC(=O)[C@@H]1CC2(CN1C(=O)CNC(=O)c1ccc3[nH]c4ccccc4c3c1)OCCO2)c1cc(C(=N)N)cs1] | (S)-7-((9H-carbazole-3- carbonyl)glycyl)-N-((R)- 1-(4- carbamimidoylthiophen- 2-yl)ethyl)-1,4-dioxa-7- azaspiro[4.4]nonane-8- carboxamide | *** | 1.05 | 575 |
| 171 | 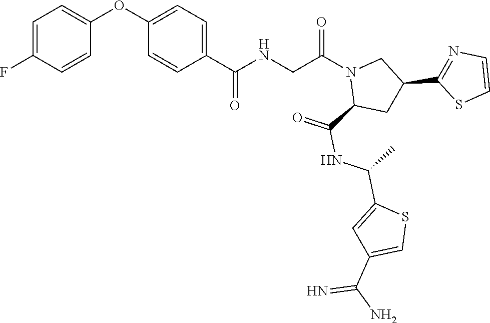 [SMILES: C[C@@H](NC(=O)[C@@H]1C[C@H](c2nccs2)CN1C(=O)CNC(=O)c1ccc(Oc2ccc(F)cc2)cc1)c1cc(C(=N)N)cs1] | (2S,4S)-N-((R)-1-(4- carbamimidoylthiophen- 2-yl)ethyl)-1-((4-(4- fluorophenoxy)benzoyl) glycyl)-4-(thiazol-2- yl)pyrrolidine-2- carboxamide | *** | 1.37 | 621 |
| 172 | 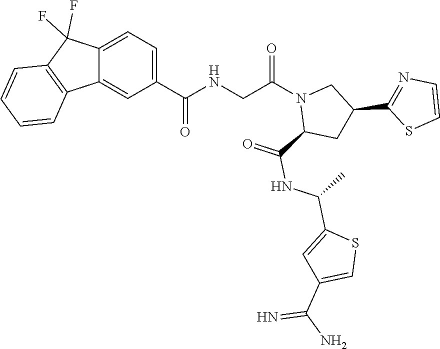 [SMILES: C[C@@H](NC(=O)[C@@H]1C[C@H](c2nccs2)CN1C(=O)CNC(=O)c1ccc2c(c1)-c1ccccc1C2(F)F)c1cc(C(=N)N)cs1] | (2S,4S)-N-((R)-1-(4- carbamimidoylthiophen- 2-yl)ethyl)-1-((9,9- difluoro-9H-fluorene-3- carbonyl)glycyl)-4- (thiazol-2-yl)pyrrolidine- 2-carboxamide | *** | 1.44 | 635 |
| 173 | 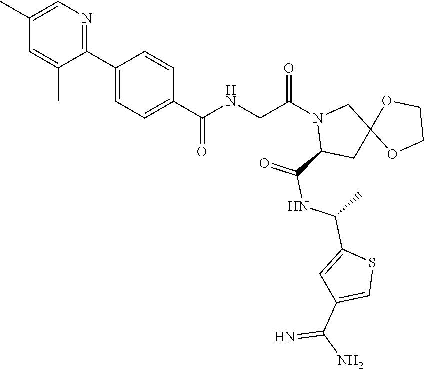 [SMILES: Cc1cnc(-c2ccc(C(=O)NCC(=O)N3CC4(C[C@H]3C(=O)N[C@H](C)c3cc(C(=N)N)cs3)OCCO4)cc2)c(C)c1] | (S)-N-((R)-1-(4- carbamimidoylthiophen- 2-yl)ethyl)-7-((4-(3,5- dimethylpyridin-2- yl)benzoyl)glycyl)-1,4- dioxa-7- azaspiro[4.4]nonane-8- carboxamide | *** | 0.47 | 591 |
| 174 | 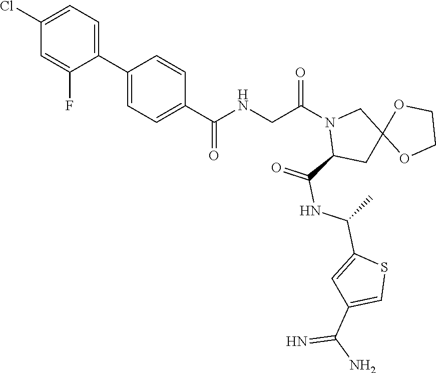 [SMILES: C[C@@H](NC(=O)[C@@H]1CC2(CN1C(=O)CNC(=O)c1ccc(-c3ccc(Cl)cc3F)cc1)OCCO2)c1cc(C(=N)N)cs1] | (S)-N-((R)-1-(4- carbamimidoylthiophen- 2-yl)ethyl)-7-((4′- chloro-2′-fluoro-[1,1′- biphenyl]-4- carbonyl)glycyl)-1,4- dioxa-7- azaspiro[4.4]nonane-8- carboxamide | *** | 1.40 | 614 |
| 175 | 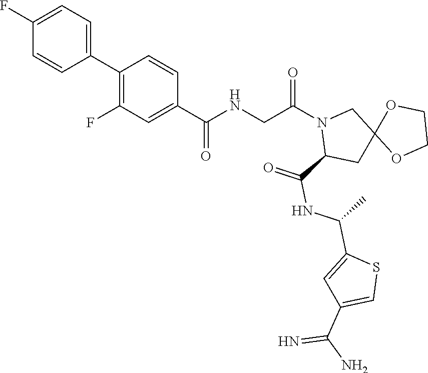 [SMILES: C[C@@H](NC(=O)[C@@H]1CC2(CN1C(=O)CNC(=O)c1ccc(-c3ccc(F)cc3)c(F)c1)OCCO2)c1cc(C(=N)N)cs1] | (S)-N-((R)-1-(4- carbamimidoylthiophen- 2-yl)ethyl)-7-((2,4′- difluoro-[1,1′-biphenyl]- 4-carbonyl)glycyl)-1,4- dioxa-7- azaspiro[4.4]nonane-8- carboxamide | *** | 1.27 | 598 |
| 176 | 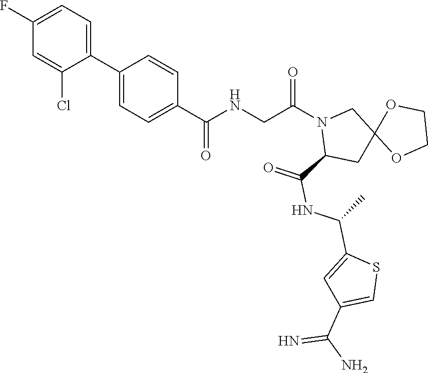 [SMILES: C[C@@H](NC(=O)[C@@H]1CC2(CN1C(=O)CNC(=O)c1ccc(-c3ccc(F)cc3Cl)cc1)OCCO2)c1cc(C(=N)N)cs1] | (S)-N-((R)-1-(4- carbamimidoylthiophen- 2-yl)ethyl)-7-((2′- chloro-4′-fluoro-[1,1′- biphenyl]-4- carbonyl)glycyl)-1,4- dioxa-7- azaspiro[4.4]nonane-8- carboxamide | *** | 1.36 | 614 |
| 177 | 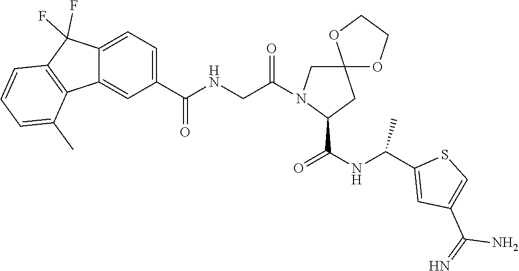 [SMILES: Cc1cccc2c1-c1cc(C(=O)NCC(=O)N3CC4(C[C@H]3C(=O)N[C@H](C)c3cc(C(=N)N)cs3)OCCO4)ccc1C2(F)F] | (S)-N-((R)-1-(4- carbamimidoylthiophen- 2-yl)ethyl)-7-((9,9- difluoro-5-methyl-9H- fluorene-3- carbonyl)glycyl)-1,4- dioxa-7- azaspiro[4.4]nonane-8- carboxamide | *** | 1.39 | 624 |
| 178 | 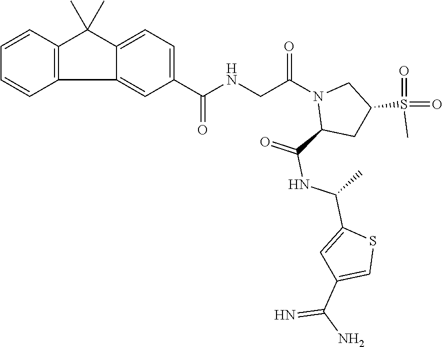 [SMILES: C[C@@H](NC(=O)[C@@H]1C[C@@H](S(C)(=O)=O)CN1C(=O)CNC(=O)c1ccc2c(c1)-c1ccccc1C2(C)C)c1cc(C(=N)N)cs1] | (2S,4R)-N-((R)-1-(4- carbamimidoylthiophen- 2-yl)ethyl)-1-((9,9- dimethyl-9H-fluorene-3- carbonyl)glycyl)-4- (methylsulfonyl) pyrrolidine-2- carboxamide | *** | 1.37 | 622 |
| 179 | 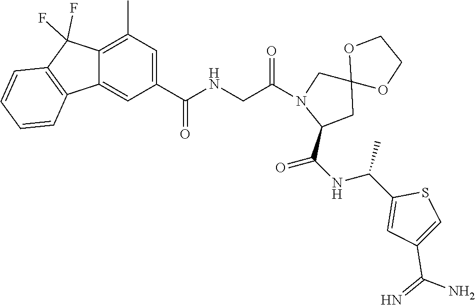 [SMILES: Cc1cc(C(=O)NCC(=O)N2CC3(C[C@H]2C(=O)N[C@H](C)c2cc(C(=N)N)cs2)OCCO3)cc2c1C(F)(F)c1ccccc1-2] | (S)-N-((R)-1-(4- carbamimidoylthiophen- 2-yl)ethyl)-7-((9,9- difluoro-1-methyl-9H- fluorene-3- carbonyl)glycyl)-1,4- dioxa-7- azaspiro[4.4]nonane-8- carboxamide | *** | 1.42 | 624 |
| 180 | 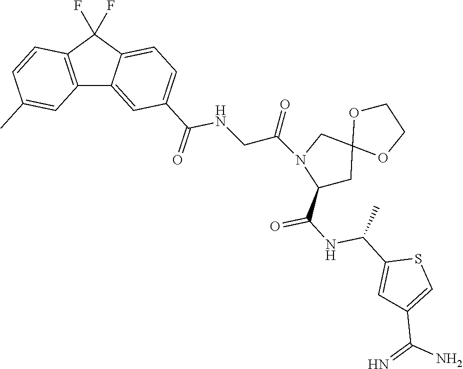 [SMILES: Cc1ccc2c(c1)-c1cc(C(=O)NCC(=O)N3CC4(C[C@H]3C(=O)N[C@H](C)c3cc(C(=N)N)cs3)OCCO4)ccc1C2(F)F] | (S)-N-((R)-1-(4- carbamimidoylthiophen- 2-yl)ethyl)-7-((9,9- difluoro-6-methyl-9H- fluorene-3- carbonyl)glycyl)-1,4- dioxa-7- azaspiro[4.4]nonane-8- carboxamide | *** | 1.45 | 624 |
| 181 | 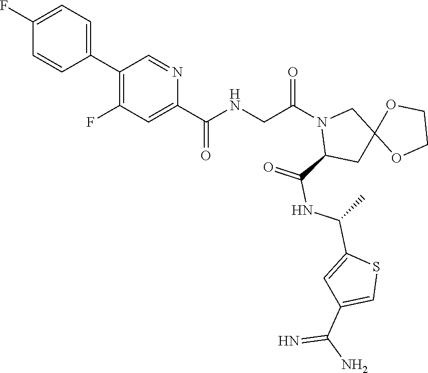 [SMILES: C[C@@H](NC(=O)[C@@H]1CC2(CN1C(=O)CNC(=O)c1cc(F)c(-c3ccc(F)cc3)cn1)OCCO2)c1cc(C(=N)N)cs1] | (S)-N-((R)-1-(4- carbamimidoylthiophen- 2-yl)ethyl)-7-((4-fluoro- 5-(4- fluorophenyl)picolinoyl) glycyl)-1,4-dioxa-7- azaspiro[4.4]nonane-8- carboxamide | *** | 1.28 | 599 |
| 182 | 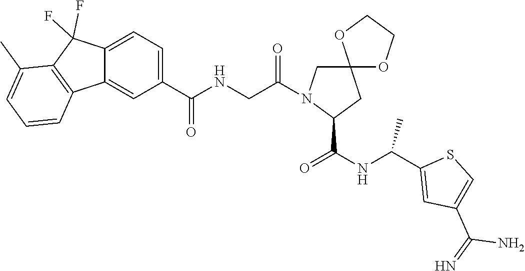 [SMILES: Cc1cccc2c1C(F)(F)c1ccc(C(=O)NCC(=O)N3CC4(C[C@H]3C(=O)N[C@H](C)c3cc(C(=N)N)cs3)OCCO4)cc1-2] | (S)-N-((R)-1-(4- carbamimidoylthiophen- 2-yl)ethyl)-7-((9,9- difluoro-8-methyl-9H- fluorene-3- carbonyl)glycyl)-1,4- dioxa-7- azaspiro[4.4 ]nonane-8- carboxamide | *** | 1.49 | 624 |
| 183 | 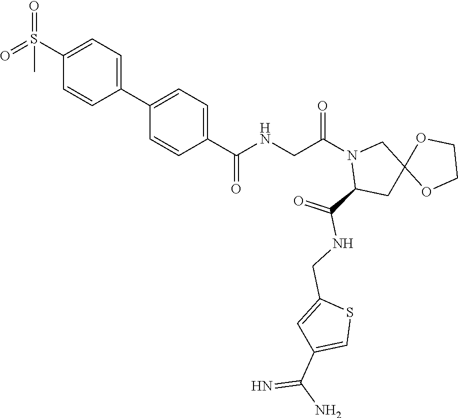 [SMILES: CS(=O)(=O)c1ccc(-c2ccc(C(=O)NCC(=O)N3CC4(C[C@H]3C(=O)NCc3cc(C(=N)N)cs3)OCCO4)cc2)cc1] | (S)-N-((4- carbamimidoylthiophen- 2-yl)methyl)-7-((4′- (methylsulfonyl)-[1,1′- biphenyl]-4- carbonyl)glycyl)-1,4- dioxa-7- azaspiro[4.4]nonane-8- carboxamide | *** | 0.90 | 626 |
| 184 | 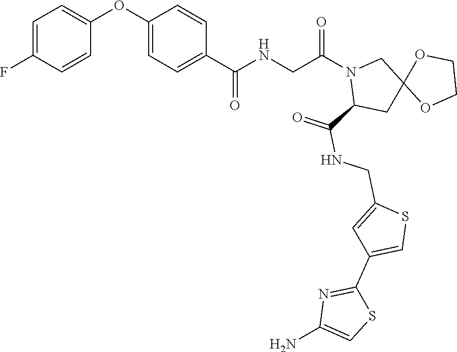 [SMILES: Nc1csc(-c2csc(CNC(=O)[C@@H]3CC4(CN3C(=O)CNC(=O)c3ccc(Oc5ccc(F)cc5)cc3)OCCO4)c2)n1] | (S)-N-((4-(4- aminothiazol-2- yl)thiophen-2- yl)methyl)-7-((4-(4- fluorophenoxy)benzoyl) glycyl)-1,4-dioxa-7- azaspiro[4.4]nonane-8- carboxamide | * | 1.96 | 638 |
| 185 | 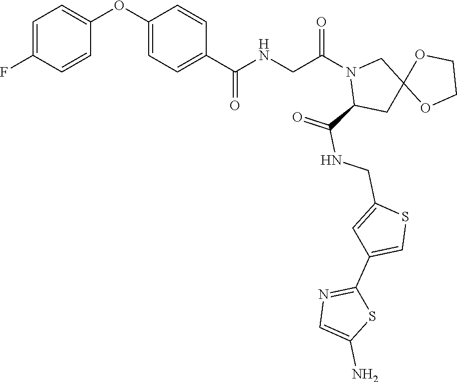 [SMILES: Nc1cnc(-c2csc(CNC(=O)[C@@H]3CC4(CN3C(=O)CNC(=O)c3ccc(Oc5ccc(F)cc5)cc3)OCCO4)c2)s1] | (S)-N-((4-(5- aminothiazol-2- yl)thiophen-2- yl)methyl)-7-((4-(4- fluorophenoxy)benzoyl) glycyl)-1,4-dioxa-7- azaspiro[4.4]nonane-8- carboxamide | *** | 1.86 | 638 |
| 186 | 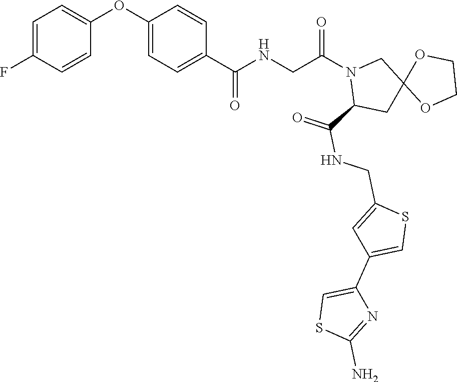 [SMILES: Nc1nc(-c2csc(CNC(=O)[C@@H]3CC4(CN3C(=O)CNC(=O)c3ccc(Oc5ccc(F)cc5)cc3)OCCO4)c2)cs1] | (S)-N-((4-(2- aminothiazol-4- yl)thiophen-2- yl)methyl)-7-((4-(4- fluorophenoxy)benzoyl) glycyl)-1,4-dioxa-7- azaspiro[4.4]nonane-8- carboxamide | * | 1.73 | 638 |
| 187 | 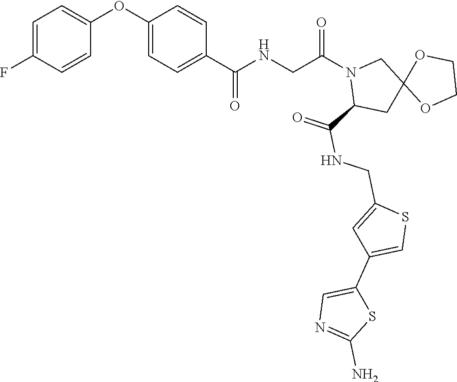 [SMILES: Nc1ncc(-c2csc(CNC(=O)[C@@H]3CC4(CN3C(=O)CNC(=O)c3ccc(Oc5ccc(F)cc5)cc3)OCCO4)c2)s1] | (S)-N-((4-(2- aminothiazol-5- yl)thiophen-2- yl)methyl)-7-((4-(4- fluorophenoxy)benzoyl) glycyl)-1,4-dioxa-7- azaspiro[4.4]nonane-8- carboxamide | * | 1.58 | 638 |
| 188 | 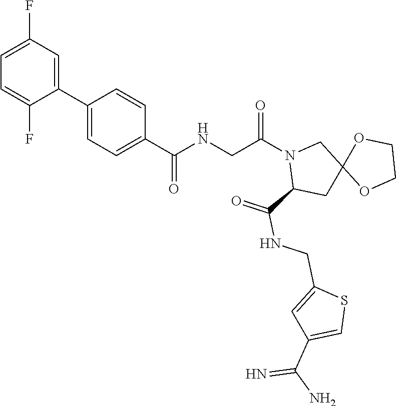 [SMILES: N=C(N)c1csc(CNC(=O)[C@@H]2CC3(CN2C(=O)CNC(=O)c2ccc(-c4cc(F)ccc4F)cc2)OCCO3)c1] | (S)-N-((4- carbamimidoylthiophen- 2-yl)methyl)-7-((2′,5′- difluoro-[1,1′-biphenyl]- 4-carbonyl)glycyl)-1,4- dioxa-7- azaspiro[4.4]nonane-8- carboxamide | *** | 1.29 | 584 |
| 189 | 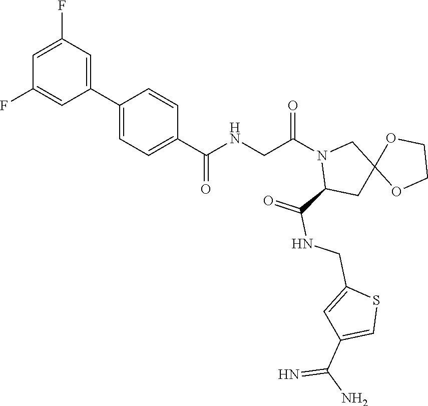 [SMILES: N=C(N)c1csc(CNC(=O)[C@@H]2CC3(CN2C(=O)CNC(=O)c2ccc(-c4cc(F)cc(F)c4)cc2)OCCO3)c1] | (S)-N-((4- carbamimidoylthiophen- 2-yl)methyl)-7-((3′,5′- difluoro-[1,1′-biphenyl]- 4-carbonyl)glycyl)-1,4- dioxa-7- azaspiro[4.4]nonane-8- carboxamide | *** | 1.37 | 584 |
| 190 | 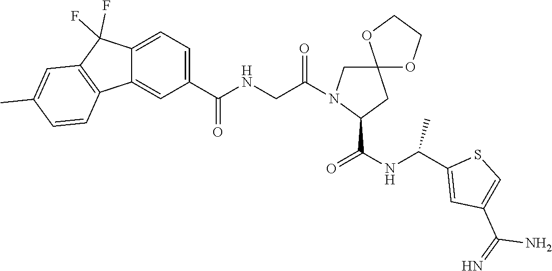 [SMILES: Cc1ccc2c(c1)C(F)(F)c1ccc(C(=O)NCC(=O)N3CC4(C[C@H]3C(=O)N[C@H](C)c3cc(C(=N)N)cs3)OCCO4)cc1-2] | (S)-N-((R)-1-(4- carbamimidoylthiophen- 2-yl)ethyl)-7-((9,9- difluoro-7-methyl-9H- fluorene-3- carbonyl)glycyl)-1,4- dioxa-7- azaspiro[4.4]nonane-8- carboxamide | *** | 1.45 | 624 |
| 191 | 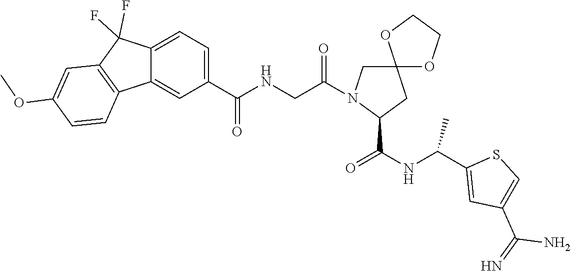 [SMILES: COc1ccc2c(c1)C(F)(F)c1ccc(C(=O)NCC(=O)N3CC4(C[C@H]3C(=O)N[C@H](C)c3cc(C(=N)N)cs3)OCCO4)cc1-2] | (S)-N-((R)-1-(4- carbamimidoylthiophen- 2-yl)ethyl)-7-((9,9- difluoro-7-methoxy-9H- fluorene-3- carbonyl)glycyl)-1,4- dioxa-7- azaspiro[4.4]nonane-8- carboxamide | *** | 1.33 | 640 |
| 192 | 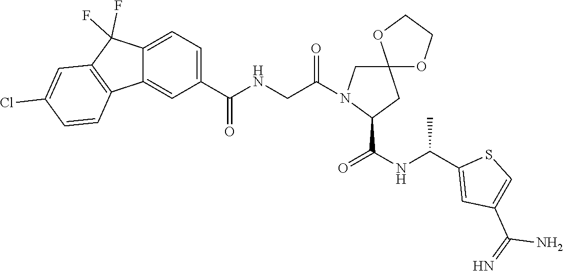 [SMILES: C[C@@H](NC(=O)[C@@H]1CC2(CN1C(=O)CNC(=O)c1ccc3c(c1)-c1ccc(Cl)cc1C3(F)F)OCCO2)c1cc(C(=N)N)cs1] | (S)-N-((R)-1-(4- carbamimidoylthiophen- 2-yl)ethyl)-7-((7-chloro- 9,9-difluoro-9H- fluorene-3- carbonyl)glycyl)-1,4- dioxa-7- azaspiro[4.4]nonane-8- carboxamide | *** | 1.49 | 644 |
| 193 | 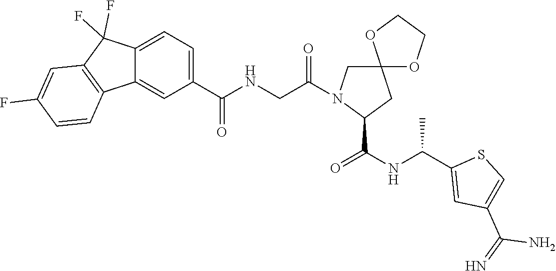 [SMILES: C[C@@H](NC(=O)[C@@H]1CC2(CN1C(=O)CNC(=O)c1ccc3c(c1)-c1ccc(F)cc1C3(F)F)OCCO2)c1cc(C(=N)N)cs1] | (S)-N-((R)-1-(4- carbamimidoylthiophen- 2-yl)ethyl)-7-((7,9,9- trifluoro-9H-fluorene-3- carbonyl)glycyl)-1,4- dioxa-7- azaspiro[4.4]nonane-8- carboxamide | *** | 1.34 | 628 |
| 194 | 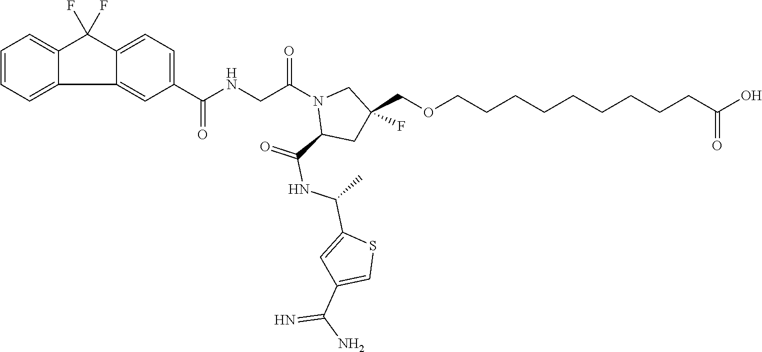 [SMILES: C[C@@H](NC(=O)[C@@H]1C[C@](F)(COCCCCCCCCCC(=O)O)CN1C(=O)CNC(=O)c1ccc2c(c1)-c1ccccc1C2(F)F)c1cc(C(=N)N)cs1] | 10-(((3R,5S)-5-(((R)-1- (4- carbamimidoylthiophen- 2-yl)ethyl)carbamoyl)- 1-((9,9-difluoro-9H- fluorene-3- carbonyl)glycyl)-3- fluoropyrrolidin-3- yl)methoxy)decanoic acid | *** | 1.84 | 770 |
| 195 | 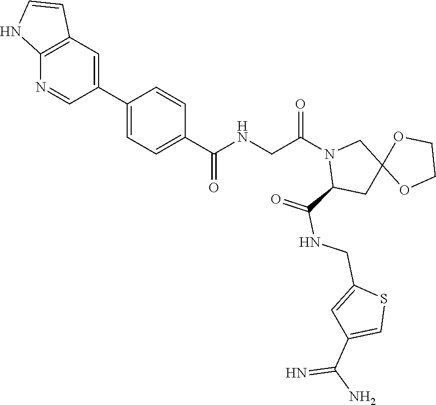 [SMILES: N=C(N)c1csc(CNC(=O)[C@@H]2CC3(CN2C(=O)CNC(=O)c2ccc(-c4cnc5[nH]ccc5c4)cc2)OCCO3)c1] | (S)-7-((4-(1H- pyrrolo[2,3-b]pyridin-5- yl)benzoyl)glycyl)-N- ((4- carbamimidoylthiophen- 2-yl)methyl)-1,4-dioxa- 7-azaspiro[4.4]nonane- 8-carboxamide | *** | 0.91 | 588 |
| 196 | 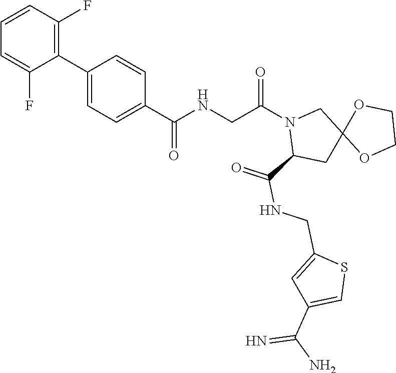 [SMILES: N=C(N)c1csc(CNC(=O)[C@@H]2CC3(CN2C(=O)CNC(=O)c2ccc(-c4c(F)cccc4F)cc2)OCCO3)c1] | (S)-N-((4- carbamimidoylthiophen- 2-yl)methyl)-7-((2′,6′- difluoro-[1,1′-biphenyl]- 4-carbonyl)glycyl)-1,4- dioxa-7- azaspiro[4.4]nonane-8- carboxamide | *** | 1.24 | 584 |
| 197 | 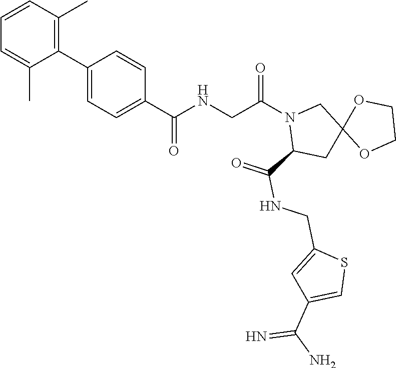 [SMILES: Cc1cccc(C)c1-c1ccc(C(=O)NCC(=O)N2CC3(C[C@H]2C(=O)NCc2cc(C(=N)N)cs2)OCCO3)cc1] | (S)-N-((4- carbamimidoylthiophen- 2-yl)methyl)-7-((2′,6′- dimethyl-[1,1′- biphenyl]-4- carbonyl)glycyl)-1,4- dioxa-7- azaspiro[4.4]nonane-8- carboxamide | *** | 1.48 | 576 |
| 198 | 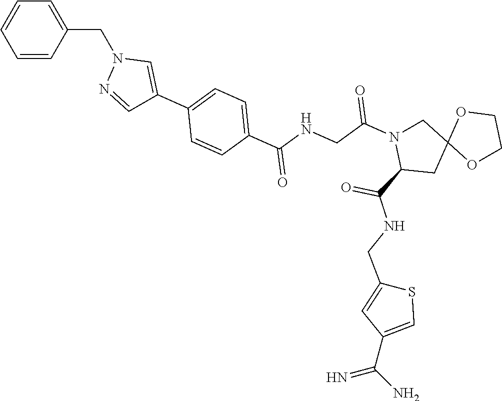 [SMILES: N=C(N)c1csc(CNC(=O)[C@@H]2CC3(CN2C(=O)CNC(=O)c2ccc(-c4cnn(Cc5ccccc5)c4)cc2)OCCO3)c1] | (S)-7-((4-(1-benzyl-1H- pyrazol-4- yl)benzoyl)glycyl)-N- ((4- carbamimidoylthiophen- 2-yl)methyl)-1,4-dioxa- 7-azaspiro[4.4]nonane- 8-carboxamide | *** | 1.19 | 628 |
| 199 | 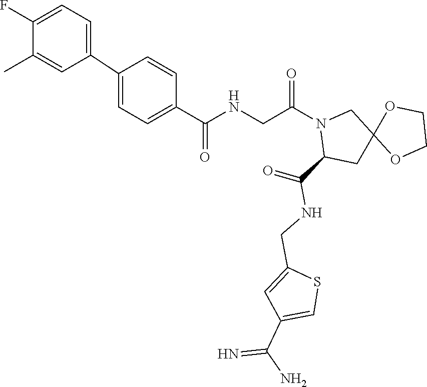 [SMILES: Cc1cc(-c2ccc(C(=O)NCC(=O)N3CC4(C[C@H]3C(=O)NCc3cc(C(=N)N)cs3)OCCO4)cc2)ccc1F] | (S)-N-((4- carbamimidoylthiophen- 2-yl)methyl)-7-((4′- fluoro-3′-methyl-[1,1′- biphenyl]-4- carbonyl)glycyl)-1,4- dioxa-7- azaspiro[4.4]nonane-8- carboxamide | *** | 1.45 | 580 |
| 200 | 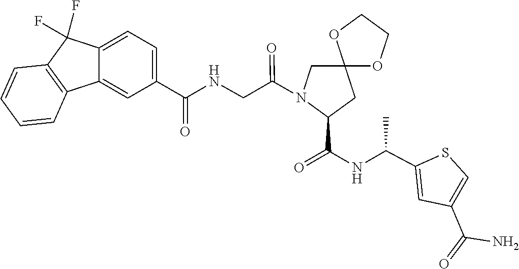 [SMILES: C[C@@H](NC(=O)[C@@H]1CC2(CN1C(=O)CNC(=O)c1ccc3c(c1)-c1ccccc1C3(F)F)OCCO2)c1cc(C(N)=O)cs1] | (S)-N-((R)-1-(4- carbamoylthiophen-2- yl)ethyl)-7-((9,9- difluoro-9H-fluorene-3- carbonyl)glycyl)-1,4- dioxa-7- azaspiro[4.4]nonane-8- carboxamide | * | 1.75 | 611 |
| 201 | 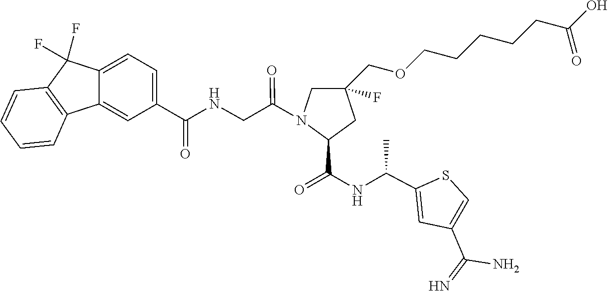 [SMILES: C[C@@H](NC(=O)[C@@H]1C[C@](F)(COCCCCCC(=O)O)CN1C(=O)CNC(=O)c1ccc2c(c1)-c1ccccc1C2(F)F)c1cc(C(=N)N)cs1] | 6-(((3R,5S)-5-(((R)-1- (4- carbamimidoylthiophen- 2-yl)ethyl)carbamoyl)- 1-((9,9-difluoro-9H- fluorene-3- carbonyl)glycyl)-3- fluoropyrrolidin-3- yl)methoxy)hexanoic acid | *** | 1.38 | 714 |
| 202 | 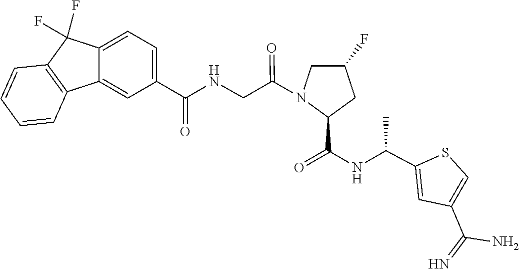 [SMILES: C[C@@H](NC(=O)[C@@H]1C[C@@H](F)CN1C(=O)CNC(=O)c1ccc2c(c1)-c1ccccc1C2(F)F)c1cc(C(=N)N)cs1] | (2S,4R)-N-((R)-1-(4- carbamimidoylthiophen- 2-yl)ethyl)-1-((9,9- difluoro-9H-fluorene-3- carbonyl)glycyl)-4- fluoropyrrolidine-2- carboxamide | *** | 1.17 | 570 |
| 203 | 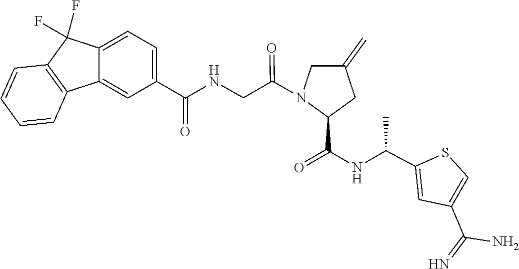 [SMILES: C=C1C[C@@H](C(=O)N[C@H](C)c2cc(C(=N)N)cs2)N(C(=O)CNC(=O)c2ccc3c(c2)-c2ccccc2C3(F)F)C1] | (S)-N-((R)-1-(4- carbamimidoylthiophen- 2-yl)ethyl)-1-((9,9- difluoro-9H-fluorene-3- carbonyl)glycyl)-4- methylenepyrrolidine-2- carboxamide | *** | 1.24 | 564 |
| 204 | 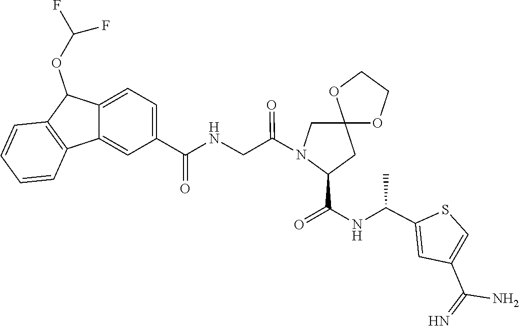 [SMILES: C[C@@H](NC(=O)[C@@H]1CC2(CN1C(=O)CNC(=O)c1ccc3c(c1)-c1ccccc1C3OC(F)F)OCCO2)c1cc(C(=N)N)cs1] | (8S)-N-((R)-1-(4- carbamimidoylthiophen- 2-yl)ethyl)-7-((9- (difluoromethoxy)-9H- fluorene-3- carbonyl)glycyl)-1,4- dioxa-7- azaspiro[4.4]nonane-8- carboxamide | *** | 1.29 | 640 |
| 205 | 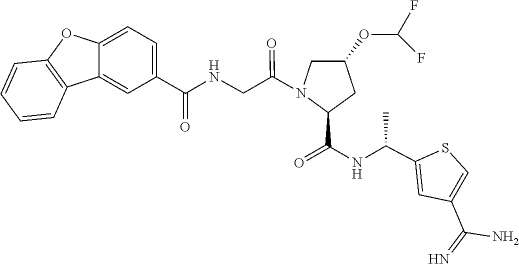 [SMILES: C[C@@H](NC(=O)[C@@H]1C[C@@H](OC(F)F)CN1C(=O)CNC(=O)c1ccc2oc3ccccc3c2c1)c1cc(C(=N)N)cs1] | (2S,4R)-N-((R)-1-(4- carbamimidoylthiophen- 2-yl)ethyl)-1- ((dibenzo[b,d]furan-2- carbonyl)glycyl)-4- (difluoromethoxy) pyrrolidine-2- carboxamide | *** | 1.19 | 584 |
| 206 | 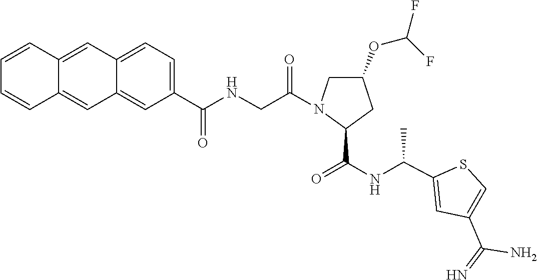 [SMILES: C[C@@H](NC(=O)[C@@H]1C[C@@H](OC(F)F)CN1C(=O)CNC(=O)c1ccc2cc3ccccc3cc2c1)c1cc(C(=N)N)cs1] | (2S,4R)-1- ((anthracene-2- carbonyl)glycyl)-N-((R)- 1-(4- carbamimidoylthiophen- 2-yl)ethyl)-4- (difluoromethoxy) pyrrolidine-2- carboxamide | *** | 1.33 | 594 |
| 207 | 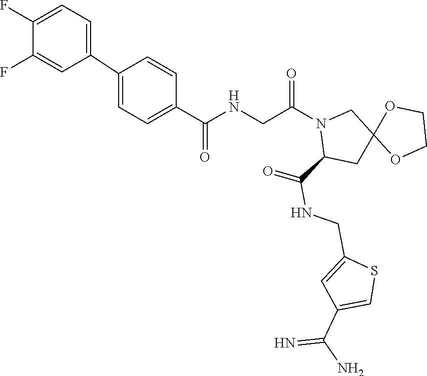 [SMILES: N=C(N)c1csc(CNC(=O)[C@@H]2CC3(CN2C(=O)CNC(=O)c2ccc(-c4ccc(F)c(F)c4)cc2)OCCO3)c1] | (S)-N-((4- carbamimidoylthiophen- 2-yl)methyl)-7-((3′,4′- difluoro-[1,1′-biphenyl]- 4-carbonyl)glycyl)-1,4- dioxa-7- azaspiro[4.4]nonane-8- carboxamide | *** | 1.34 | 584 |
| 208 | 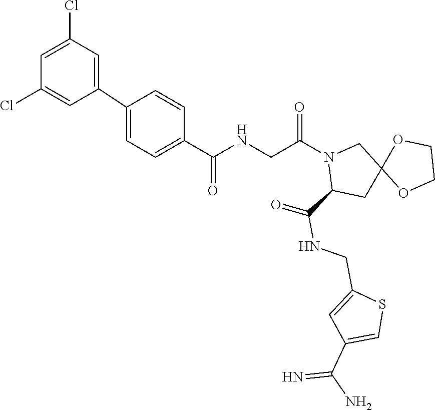 [SMILES: N=C(N)c1csc(CNC(=O)[C@@H]2CC3(CN2C(=O)CNC(=O)c2ccc(-c4cc(Cl)cc(Cl)c4)cc2)OCCO3)c1] | (S)-N-((4- carbamimidoylthiophen- 2-yl)methyl)-7-((3′,5′- dichloro-[1,1′-biphenyl]- 4-carbonyl)glycyl)-1,4- dioxa-7- azaspiro[4.4]nonane-8- carboxamide | *** | 1.67 | 616 |
| 209 | 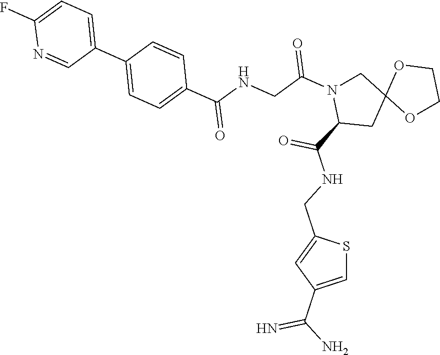 [SMILES: N=C(N)c1csc(CNC(=O)[C@@H]2CC3(CN2C(=O)CNC(=O)c2ccc(-c4ccc(F)nc4)cc2)OCCO3)c1] | (S)-N-((4- carbamimidoylthiophen- 2-yl)methyl)-7-((4-(6- fluoropyridin-3- yl)benzoyl)glycyl)-1,4- dioxa-7- azaspiro[4.4]nonane-8- carboxamide | *** | 0.93 | 567 |
| 210 | 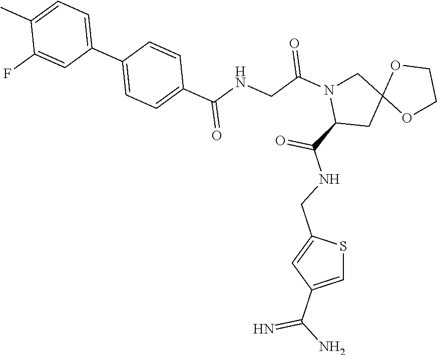 [SMILES: Cc1ccc(-c2ccc(C(=O)NCC(=O)N3CC4(C[C@H]3C(=O)NCc3cc(C(=N)N)cs3)OCCO4)cc2)cc1F] | (S)-N-((4- carbamimidoylthiophen- 2-yl)methyl)-7-((3′- fluoro-4′-methyl-[1,1′- biphenyl]-4- carbonyl)glycyl)-1,4- dioxa-7- azaspiro[4.4]nonane-8- carboxamide | *** | 1.48 | 580 |
| 211 | 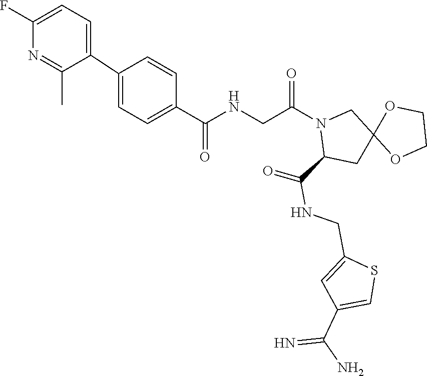 [SMILES: Cc1nc(F)ccc1-c1ccc(C(=O)NCC(=O)N2CC3(C[C@H]2C(=O)NCc2cc(C(=N)N)cs2)OCCO3)cc1] | (S)-N-((4- carbamimidoylthiophen- 2-yl)methyl)-7-((4-(6- fluoro-2-methylpyridin- 3-yl)benzoyl)glycyl)- 1,4-dioxa-7- azaspiro[4.4]nonane-8- carboxamide | *** | 0.92 | 581 |
| 212 | 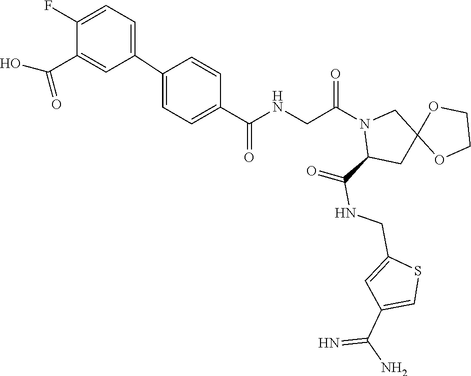 [SMILES: N=C(N)c1csc(CNC(=O)[C@@H]2CC3(CN2C(=O)CNC(=O)c2ccc(-c4ccc(F)c(C(=O)O)c4)cc2)OCCO3)c1] | (S)-4′-((2-(8-(((4- carbamimidoylthiophen- 2- yl)methyl)carbamoyl)- 1,4-dioxa-7- azaspiro[4.4]nonan-7- yl)-2- oxoethyl)carbamoyl)-4- fluoro-[1,1′-biphenyl]-3- carboxylic acid | *** | 0.80 | 610 |
| 213 | 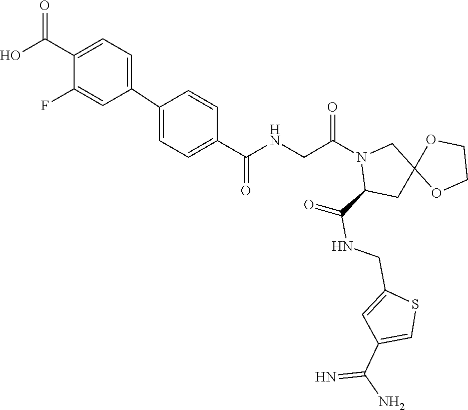 [SMILES: N=C(N)c1csc(CNC(=O)[C@@H]2CC3(CN2C(=O)CNC(=O)c2ccc(-c4ccc(C(=O)O)c(F)c4)cc2)OCCO3)c1] | (S)-4′-((2-(8-(((4- carbamimidoylthiophen- 2- yl)methyl)carbamoyl)- 1,4-dioxa-7- azaspiro[4.4]nonan-7- yl)-2- oxoethyl)carbamoyl)-3- fluoro-[1,1′-biphenyl]-4- carboxylic acid | * | 0.75 | 610 |
| 214 | 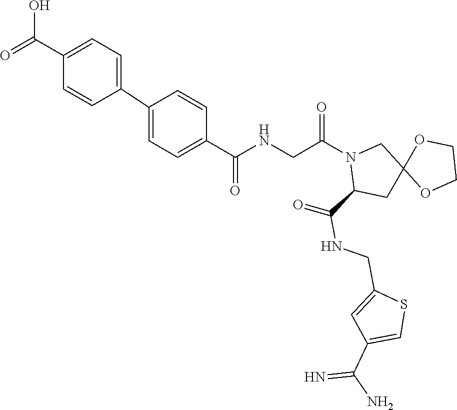 [SMILES: N=C(N)c1csc(CNC(=O)[C@@H]2CC3(CN2C(=O)CNC(=O)c2ccc(-c4ccc(C(=O)O)cc4)cc2)OCCO3)c1] | (S)-4′-((2-(8-(((4- carbamimidoylthiophen- 2- yl)methyl)carbamoyl)- 1,4-dioxa-7- azaspiro[4.4]nonan-7- yl)-2- oxoethyl) carbamoyl)- [1,1′-biphenyl]-4- carboxylic acid | *** | 0.78 | 592 |
| 215 | 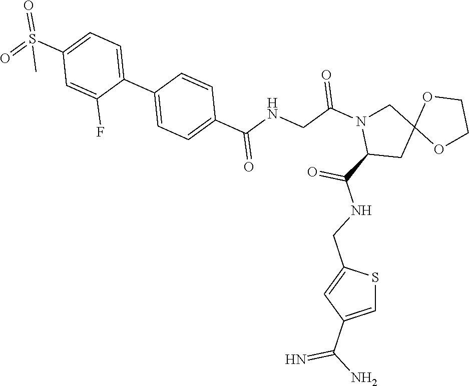 [SMILES: CS(=O)(=O)c1ccc(-c2ccc(C(=O)NCC(=O)N3CC4(C[C@H]3C(=O)NCc3cc(C(=N)N)cs3)OCCO4)cc2)c(F)c1] | (S)-N-((4- carbamimidoylthiophen- 2-yl)methyl)-7-((2′- fluoro-4′- (methylsulfonyl)-[1,1′- biphenyl]-4- carbonyl)glycyl)-1,4- dioxa-7- azaspiro[4.4]nonane-8- carboxamide | *** | 0.87 | 644 |
| 216 | 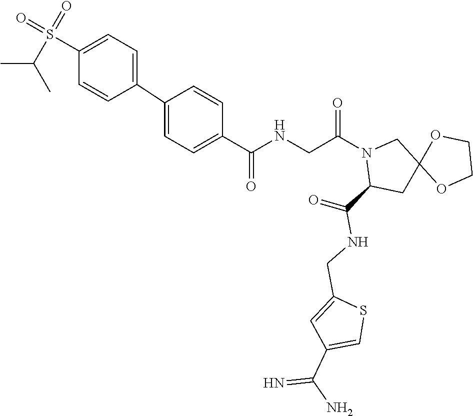 [SMILES: CC(C)S(=O)(=O)c1ccc(-c2ccc(C(=O)NCC(=O)N3CC4(C[C@H]3C(=O)NCc3cc(C(=N)N)cs3)OCCO4)cc2)cc1] | (S)-N-((4- carbamimidoylthiophen- 2-yl)methyl)-7-((4′- (isopropylsulfonyl)- [1,1′-biphenyl]-4- carbonyl)glycyl)-1,4- dioxa-7- azaspiro[4.4]nonane-8- carboxamide | *** | 1.02 | 654 |
| 217 | 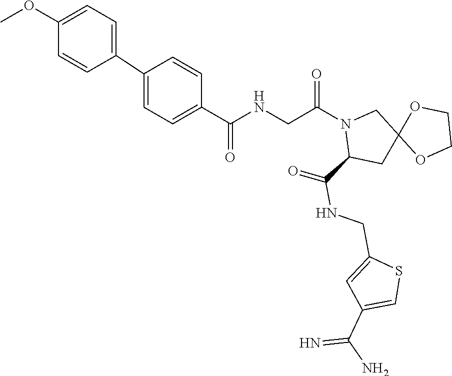 [SMILES: COc1ccc(-c2ccc(C(=O)NCC(=O)N3CC4(C[C@H]3C(=O)NCc3cc(C(=N)N)cs3)OCCO4)cc2)cc1] | (S)-N-((4- carbamimidoylthiophen- 2-yl)methyl)-7-((4′- methoxy-[1,1′- biphenyl]-4- carbonyl)glycyl)-1,4- dioxa-7- azaspiro[4.4]nonane-8- carboxamide | *** | 1.12 | 578 |
| 218 | 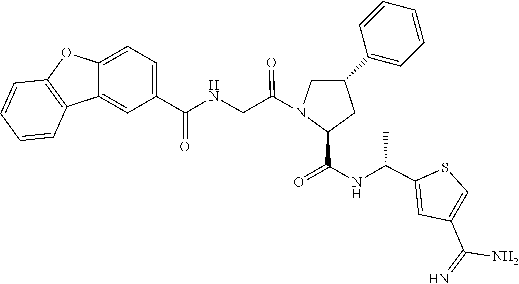 [SMILES: C[C@@H](NC(=O)[C@@H]1C[C@@H](c2ccccc2)CN1C(=O)CNC(=O)c1ccc2oc3ccccc3c2c1)c1cc(C(=N)N)cs1] | (2S,4S)-N-((R)-1-(4- carbamimidoylthiophen- 2-yl)ethyl)-1- ((dibenzo[b,d]furan-2- carbonyl)glycyl)-4- phenylpyrrolidine-2- carboxamide | *** | 1.45 | 594 |
| 219 | 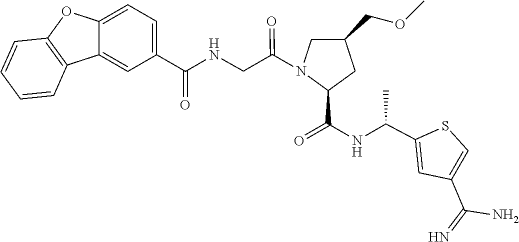 [SMILES: COC[C@H]1C[C@@H](C(=O)N[C@H](C)c2cc(C(=N)N)cs2)N(C(=O)CNC(=O)c2ccc3oc4ccccc4c3c2)C1] | (2S,4S)-N-((R)-1-(4- carbamimidoylthiophen- 2-yl)ethyl)-1- ((dibenzo[b,d]furan-2- carbonyl)glycyl)-4- (methoxymethyl) pyrrolidine-2- carboxamide | *** | 1.09 | 562 |
| 220 | 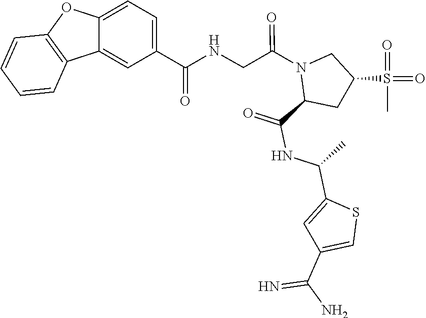 [SMILES: C[C@@H](NC(=O)[C@@H]1C[C@@H](S(C)(=O)=O)CN1C(=O)CNC(=O)c1ccc2oc3ccccc3c2c1)c1cc(C(=N)N)cs1] | (2S,4R)-N-((R)-1-(4- carbamimidoylthiophen- 2-yl)ethyl)-1- ((dibenzo[b,d]furan-2- carbonyl)glycyl)-4- (methylsulfonyl) pyrrolidine-2- carboxamide | *** | 1.02 | 596 |
| 221 | 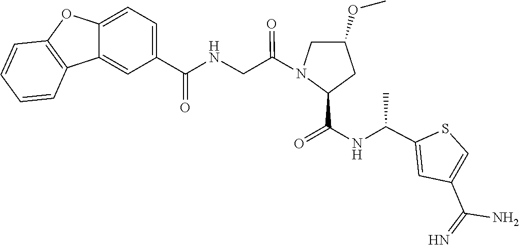 [SMILES: CO[C@@H]1C[C@@H](C(=O)N[C@H](C)c2cc(C(=N)N)cs2)N(C(=O)CNC(=O)c2ccc3oc4ccccc4c3c2)C1] | (2S,4R)-N-((R)-1-(4- carbamimidoylthiophen- 2-yl)ethyl)-1- ((dibenzo[b,d]furan-2- carbonyl)glycyl)-4- methoxypyrrolidine-2- carboxamide | *** | 1.02 | 548 |
| 222 | 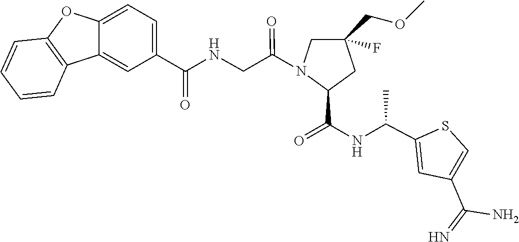 [SMILES: COC[C@@]1(F)C[C@@H](C(=O)N[C@H](C)c2cc(C(=N)N)cs2)N(C(=O)CNC(=O)c2ccc3oc4ccccc4c3c2)C1] | (2S,4R)-N-((R)-1-(4- carbamimidoylthiophen- 2-yl)ethyl)-1- ((dibenzo[b,d]furan-2- carbonyl)glycyl)-4- fluoro-4- (methoxymethyl) pyrrolidine-2- carboxamide | *** | 1.12 | 580 |
| 223 | 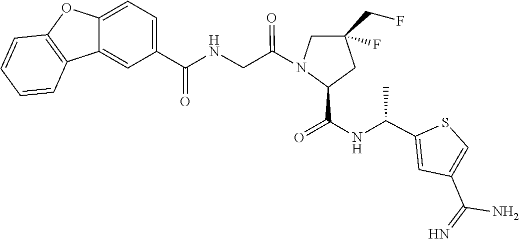 [SMILES: C[C@@H](NC(=O)[C@@H]1C[C@](F)(CF)CN1C(=O)CNC(=O)c1ccc2oc3ccccc3c2c1)c1cc(C(=N)N)cs1] | (2S,4R)-N-((R)-1-(4- carbamimidoylthiophen- 2-yl)ethyl)-1- ((dibenzo[b,d]furan-2- carbonyl)glycyl)-4- fluoro-4- (fluoromethyl)pyrrolidine- 2-carboxamide | *** | 1.14 | 568 |
| 224 | 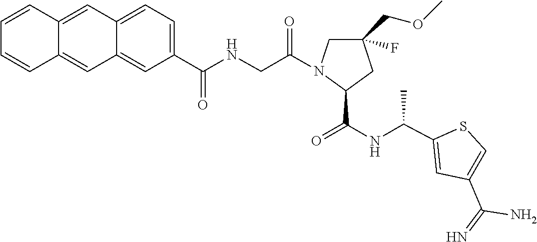 [SMILES: COC[C@@]1(F)C[C@@H](C(=O)N[C@H](C)c2cc(C(=N)N)cs2)N(C(=O)CNC(=O)c2ccc3cc4ccccc4cc3c2)C1] | (2S,4R)-1- ((anthracene-2- carbonyl)glycyl)-N-((R)- 1-(4- carbamimidoylthiophen- 2-yl)ethyl)-4-fluoro-4- (methoxymethyl) pyrrolidine-2- carboxamide | *** | 1.28 | 590 |
| 225 | 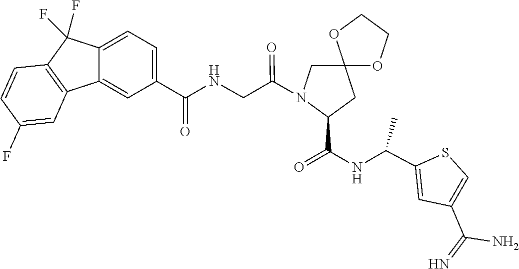 [SMILES: C[C@@H](NC(=O)[C@@H]1CC2(CN1C(=O)CNC(=O)c1ccc3c(c1)-c1cc(F)ccc1C3(F)F)OCCO2)c1cc(C(=N)N)cs1] | (S)-N-((R)-1-(4- carbamimidoylthiophen- 2-yl)ethyl)-7-((6,9,9- trifluoro-9H-fluorene-3- carbonyl)glycyl)-1,4- dioxa-7- azaspiro[4.4]nonane-8- carboxamide | *** | 1.21 | 628 |
| 226 | 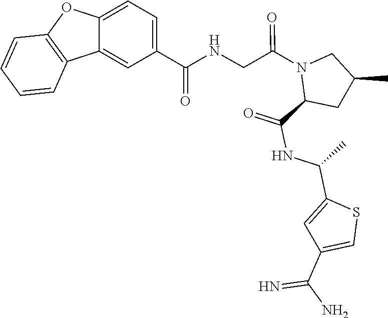 [SMILES: C[C@H]1C[C@@H](C(=O)N[C@H](C)c2cc(C(=N)N)cs2)N(C(=O)CNC(=O)c2ccc3oc4ccccc4c3c2)C1] | (2S,4S)-N-((R)-1-(4- carbamimidoylthiophen- 2-yl)ethyl)-1- ((dibenzo[b,d]furan-2- carbonyl)glycyl)-4- methylpyrrolidine-2- carboxamide | *** | 1.06 | 532 |
| 227 | 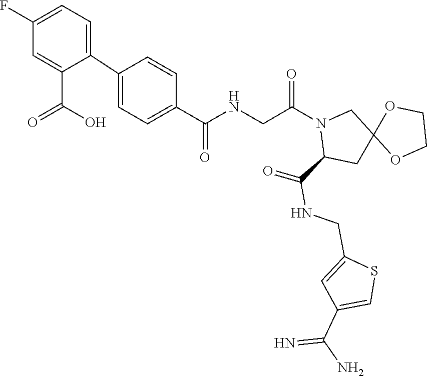 [SMILES: N=C(N)c1csc(CNC(=O)[C@@H]2CC3(CN2C(=O)CNC(=O)c2ccc(-c4ccc(F)cc4C(=O)O)cc2)OCCO3)c1] | (S)-4′-((2-(8-(((4- carbamimidoylthiophen- 2- yl)methyl)carbamoyl)- 1,4-dioxa-7- azaspiro[4.4]nonan-7- yl)-2- oxoethyl)carbamoyl)-4- fluoro-[1,1′-biphenyl]-2- carboxylic acid | *** | 0.91 (A) | 610 |
| 228 | 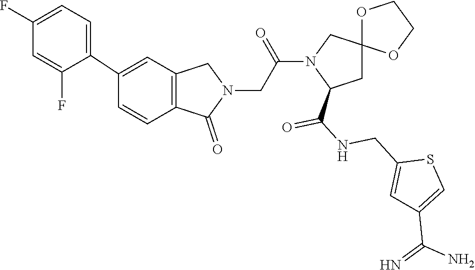 [SMILES: N=C(N)c1csc(CNC(=O)[C@@H]2CC3(CN2C(=O)CN2Cc4cc(-c5ccc(F)cc5F)ccc4C2=O)OCCO3)c1] | (S)-N-((4- carbamimidoylthiophen- 2-yl)methyl)-7-(2-(5- (2,4-difluorophenyl)-1- oxoisoindolin-2- yl)acetyl)-1,4-dioxa-7- azaspiro[4.4]nonane-8- carboxamide | *** | 1.21 | 596 |
| 229 | 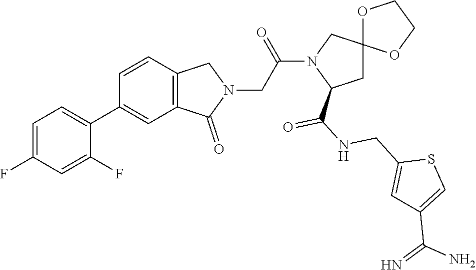 [SMILES: N=C(N)c1csc(CNC(=O)[C@@H]2CC3(CN2C(=O)CN2Cc4ccc(-c5ccc(F)cc5F)cc4C2=O)OCCO3)c1] | (S)-N-((4- carbamimidoylthiophen- 2-yl)methyl)-7-(2-(6- (2,4-difluorophenyl)-1- oxoisoindolin-2- yl)acetyl)-1,4-dioxa-7- azaspiro[4.4]nonane-8- carboxamide | *** | 1.21 | 596 |
| 230 | 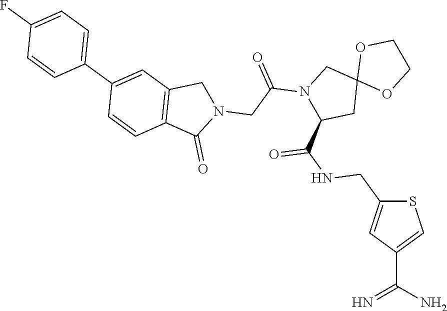 [SMILES: N=C(N)c1csc(CNC(=O)[C@@H]2CC3(CN2C(=O)CN2Cc4cc(-c5ccc(F)cc5)ccc4C2=O)OCCO3)c1] | (S)-N-((4- carbamimidoylthiophen- 2-yl)methyl)-7-(2-(5-(4- fluorophenyl)-1- oxoisoindolin-2- yl)acetyl)-1,4-dioxa-7- azaspiro[4.4]nonane-8- carboxamide | *** | 1.15 | 578 |
| 231 | 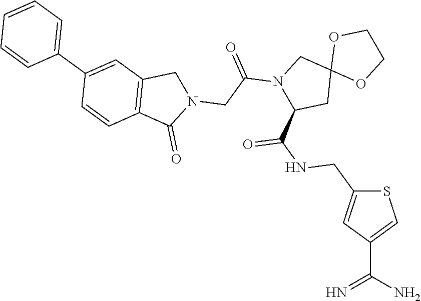 [SMILES: N=C(N)c1csc(CNC(=O)[C@@H]2CC3(CN2C(=O)CN2Cc4cc(-c5ccccc5)ccc4C2=O)OCCO3)c1] | (S)-N-((4- carbamimidoylthiophen- 2-yl)methyl)-7-(2-(1- oxo-5-phenylisoindolin- 2-yl)acetyl)-1,4-dioxa-7- azaspiro[4.4]nonane-8- carboxamide | *** | 1.11 | 560 |
| 232 | 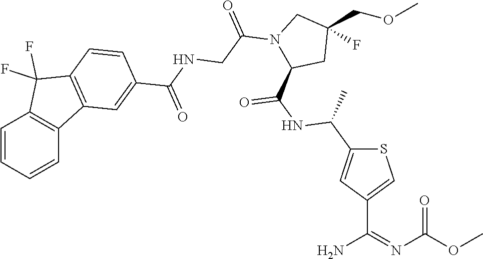 [SMILES: COC[C@@]1(F)C[C@@H](C(=O)N[C@H](C)c2cc(/C(N)=N\C(=O)OC)cs2)N(C(=O)CNC(=O)c2ccc3c(c2)-c2ccccc2C3(F)F)C1] | (E)-methyl (amino(5- ((R)-1-((2S,4R)-1-((9,9- difluoro-9H-fluorene-3- carbonyl)glycyl)-4- fluoro-4- (methoxymethyl) pyrrolidine-2- carboxamido)ethyl) thiophen-3- yl)methylene)carbamate | *** | 1.41 | 672 |
| 233 | 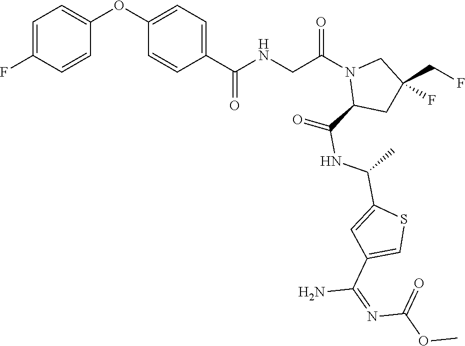 [SMILES: COC(=O)/N=C(/N)c1csc([C@@H](C)NC(=O)[C@@H]2C[C@](F)(CF)CN2C(=O)CNC(=O)c2ccc(Oc3ccc(F)cc3)cc2)c1] | (E)-methyl (amino(5- ((R)-1-((2S,4R)-4- fluoro-4-(fluoromethyl)- 1-((4-(4- fluorophenoxy)benzoyl) glycyl)pyrrolidine-2- carboxamido)ethyl) thiophen-3- yl)methylene)carbamate | *** | 1.38 | 646 |
| 234 | 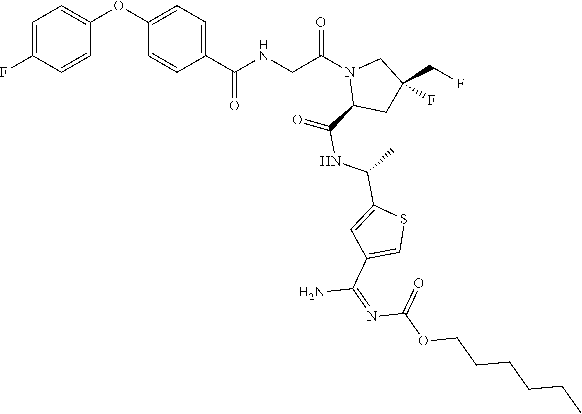 [SMILES: CCCCCCOC(=O)/N=C(/N)c1csc([C@@H](C)NC(=O)[C@@H]2C[C@](F)(CF)CN2C(=O)CNC(=O)c2ccc(Oc3ccc(F)cc3)cc2)c1] | (E)-hexyl (amino(5- ((R)-1-((2S,4R)-4- fluoro-4-(fluoromethyl)- 1-((4-(4- fluorophenoxy)benzoyl) glycyl)pyrrolidine-2- carboxamido)ethyl) thiophen-3- yl)methylene)carbamate | *** | 2.11 | 716 |
| 235 | 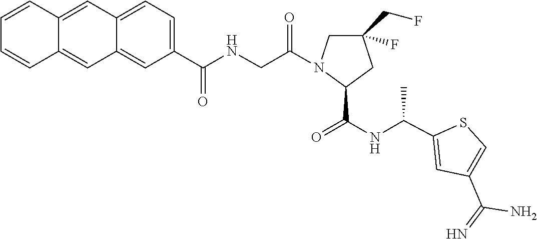 [SMILES: C[C@@H](NC(=O)[C@@H]1C[C@](F)(CF)CN1C(=O)CNC(=O)c1ccc2cc3ccccc3cc2c1)c1cc(C(=N)N)cs1] | (2S,4R)-1- ((anthracene-2- carbonyl)glycyl)-N-((R)- 1-(4- carbamimidoylthiophen- 2-yl)ethyl)-4-fluoro-4- (fluoromethyl)pyrrolidine- 2-carboxamide | *** | 1.29 | 578 |
| 236 | 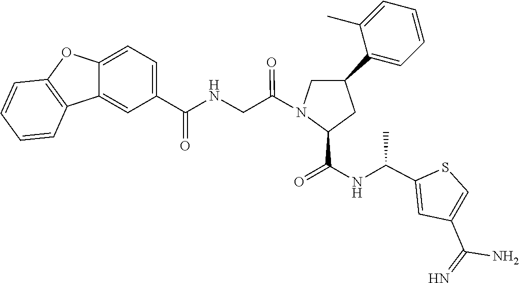 [SMILES: Cc1ccccc1[C@H]1C[C@@H](C(=O)N[C@H](C)c2cc(C(=N)N)cs2)N(C(=O)CNC(=O)c2ccc3oc4ccccc4c3c2)C1] | (2S,4R)-N-((R)-1-(4- carbamimidoylthiophen- 2-yl)ethyl)-1- ((dibenzo[b,d]furan-2- carbonyl)glycyl)-4-(o- tolyl)pyrrolidine-2- carboxamide | *** | 1.52 | 608 |
| 237 | 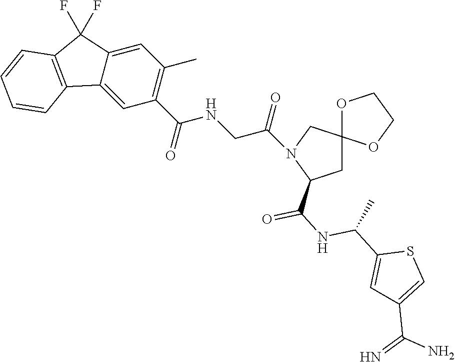 [SMILES: Cc1cc2c(cc1C(=O)NCC(=O)N1CC3(C[C@H]1C(=O)N[C@H](C)c1cc(C(=N)N)cs1)OCCO3)-c1ccccc1C2(F)F] | (S)-N-((R)-1-(4- carbamimidoylthiophen- 2-yl)ethyl)-7-((9,9- difluoro-2-methyl-9H- fluorene-3- carbonyl)glycyl)-1,4- dioxa-7- azaspiro[4.4]nonane-8- carboxamide | *** | 1.29 | 624 |
| 238 | 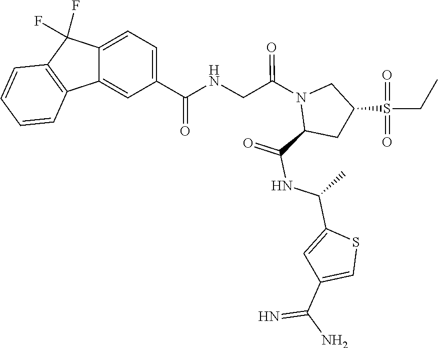 [SMILES: CCS(=O)(=O)[C@@H]1C[C@@H](C(=O)N[C@H](C)c2cc(C(=N)N)cs2)N(C(=O)CNC(=O)c2ccc3c(c2)-c2ccccc2C3(F)F)C1] | (2S,4R)-N-((R)-1-(4- carbamimidoylthiophen- 2-yl)ethyl)-1-((9,9- difluoro-9H-fluorene-3- carbonyl)glycyl)-4- (ethylsulfonyl)pyrrolidine- 2-carboxamide | *** | 1.20 | 644 |
| 239 | 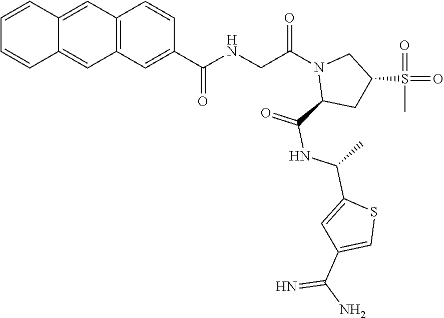 [SMILES: C[C@@H](NC(=O)[C@@H]1C[C@@H](S(C)(=O)=O)CN1C(=O)CNC(=O)c1ccc2cc3ccccc3cc2c1)c1cc(C(=N)N)cs1] | (2S,4R)-1- ((anthracene-2- carbonyl)glycyl)-N-((R)- 1-(4- carbamimidoylthiophen- 2-yl)ethyl)-4- (methylsulfonyl) pyrrolidine-2- carboxamide | *** | 1.17 | 606 |
| 240 | 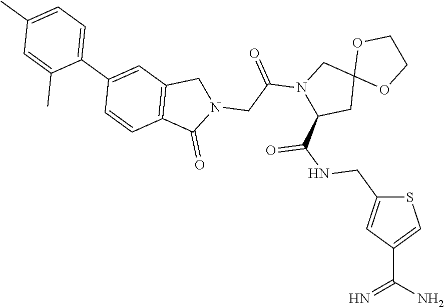 [SMILES: Cc1ccc(-c2ccc3c(c2)CN(CC(=O)N2CC4(C[C@H]2C(=O)NCc2cc(C(=N)N)cs2)OCCO4)C3=O)c(C)c1] | (S)-N-((4- carbamimidoylthiophen- 2-yl)methyl)-7-(2-(5- (2,4-dimethylphenyl)-1- oxoisoindolin-2- yl)acetyl)-1,4-dioxa-7- azaspiro[4.4]nonane-8- carboxamide | *** | 1.43 | 588 |
| 241 | 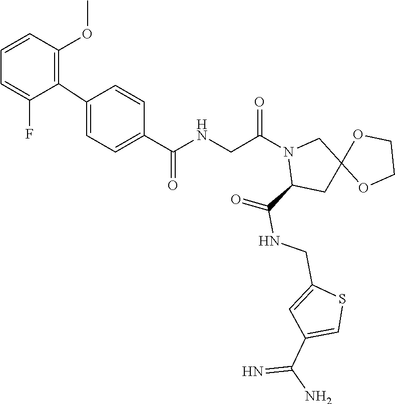 [SMILES: COc1cccc(F)c1-c1ccc(C(=O)NCC(=O)N2CC3(C[C@H]2C(=O)NCc2cc(C(=N)N)cs2)OCCO3)cc1] | (S)-N-((4- carbamimidoylthiophen- 2-yl)methyl)-7-((2′- fluoro-6′-methoxy-[1,1′- biphenyl]-4- carbonyl)glycyl)-1,4- dioxa-7- azaspiro[4.4]nonane-8- carboxamide | *** | 1.14 | 596 |
| 242 | 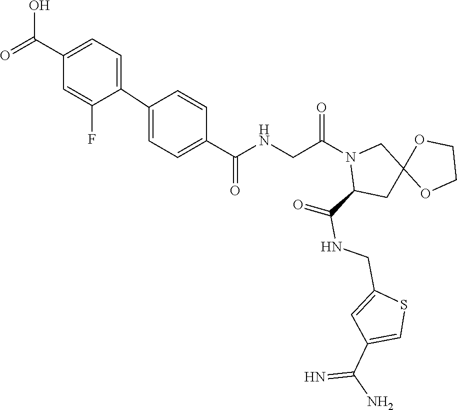 [SMILES: N=C(N)c1csc(CNC(=O)[C@@H]2CC3(CN2C(=O)CNC(=O)c2ccc(-c4ccc(C(=O)O)cc4F)cc2)OCCO3)c1] | (S)-4′-((2-(8-(((4- carbamimidoylthiophen- 2- yl)methyl)carbamoyl)- 1,4-dioxa-7- azaspiro[4.4]nonan-7- yl)-2- oxoethyl)carbamoyl)-2- fluoro-[1,1′-biphenyl]-4- carboxylic acid | *** | 0.82 | 610 |
| 243 | 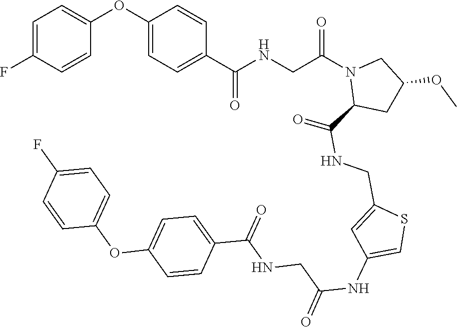 [SMILES: CO[C@@H]1C[C@@H](C(=O)NCc2cc(NC(=O)CNC(=O)c3ccc(Oc4ccc(F)cc4)cc3)cs2)N(C(=O)CNC(=O)c2ccc(Oc3ccc(F)cc3)cc2)C1] | (2S,4R)-N-((4-(2-(4-(4- fluorophenoxy)benzamido) acetamido)thiophen- 2-yl)methyl)-1-((4-(4- fluorophenoxy)benzoyl) glycyl)-4- methoxypyrrolidine-2- carboxamide | * | 2.42 | 799 |
| 244 | 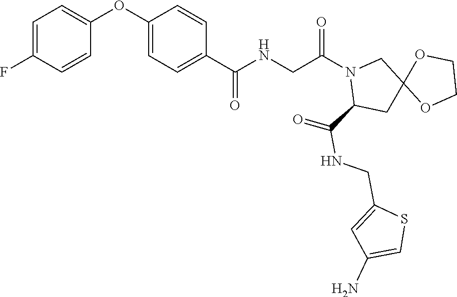 [SMILES: Nc1csc(CNC(=O)[C@@H]2CC3(CN2C(=O)CNC(=O)c2ccc(Oc4ccc(F)cc4)cc2)OCCO3)c1] | (S)-N-((4- aminothiophen-2- yl)methyl)-7-((4-(4- fluorophenoxy)benzoyl) glycyl)-1,4-dioxa-7- azaspiro[4.4]nonane-8- carboxamide | * | 1.38 | 555 |
| 245 | 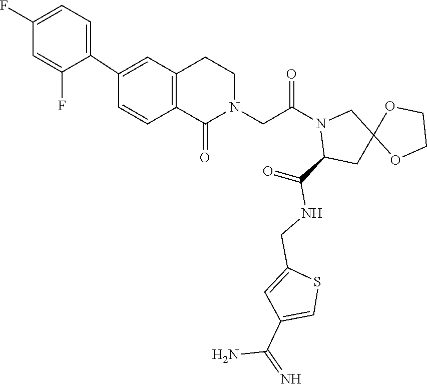 [SMILES: N=C(N)c1csc(CNC(=O)[C@@H]2CC3(CN2C(=O)CN2CCc4cc(-c5ccc(F)cc5F)ccc4C2=O)OCCO3)c1] | (S)-N-((4- carbamimidoylthiophen- 2-yl)methyl)-7-(2-(6- (2,4-difluorophenyl)-1- oxo-3,4- dihydroisoquinolin- 2(1H)-yl)acetyl)-1,4- dioxa-7- azaspiro[4.4]nonane-8- carboxamide | *** | 1.30 | 610 |
| 246 | 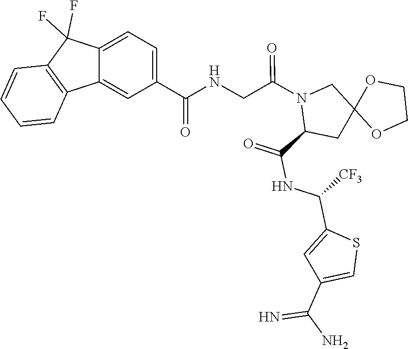 [SMILES: N=C(N)c1csc([C@H](NC(=O)[C@@H]2CC3(CN2C(=O)CNC(=O)c2ccc4c(c2)-c2ccccc2C4(F)F)OCCO3)C(F)(F)F)c1] | (S)-N-((R)-1-(4- carbamimidoylthiophen- 2-yl)-2,2,2- trifluoroethyl)-7-((9,9- difluoro-9H-fluorene-3- carbonyl)glycyl)-1,4- dioxa-7- azaspiro[4.4]nonane-8- carboxamide | *** | 1.43 | 664 |
| 247 | 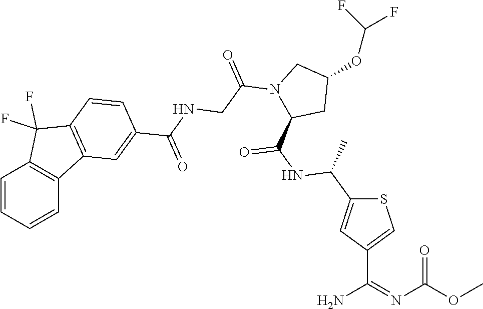 [SMILES: COC(=O)/N=C(/N)c1csc([C@@H](C)NC(=O)[C@@H]2C[C@@H](OC(F)F)CN2C(=O)CNC(=O)c2ccc3c(c2)-c2ccccc2C3(F)F)c1] | (E)-methyl (amino(5- ((R)-1-((2S,4R)-1-((9,9- difluoro-9H-fluorene-3- carbonyl)glycyl)-4- (difluoromethoxy) pyrrolidine-2- carboxamido)ethyl) thiophen-3- yl)methylene)carbamate | *** | 1.50 | 676 |
| 248 | 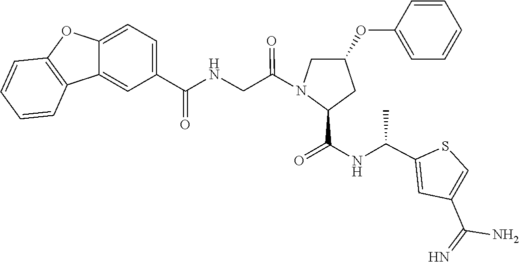 [SMILES: C[C@@H](NC(=O)[C@@H]1C[C@@H](Oc2ccccc2)CN1C(=O)CNC(=O)c1ccc2oc3ccccc3c2c1)c1cc(C(=N)N)cs1] | (2S,4R)-N-((R)-1-(4- carbamimidoylthiophen- 2-yl)ethyl)-1- ((dibenzo[b,d]furan-2- carbonyl)glycyl)-4- phenoxypyrrolidine-2- carboxamide | *** | 1.46 | 610 |
| 249 | 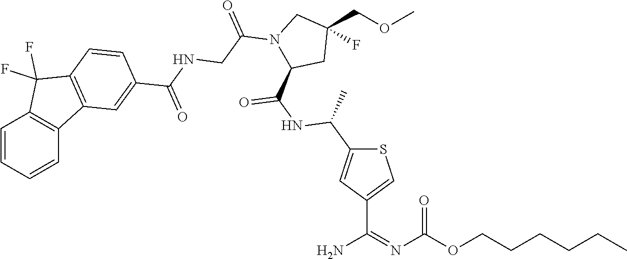 [SMILES: CCCCCCOC(=O)/N=C(/N)c1csc([C@@H](C)NC(=O)[C@@H]2C[C@](F)(COC)CN2C(=O)CNC(=O)c2ccc3c(c2)-c2ccccc2C3(F)F)c1] | (E)-hexyl (amino(5- ((R)-1-((2S,4R)-1-((9,9- difluoro-9H-fluorene-3- carbonyl)glycyl)-4- fluoro-4- (methoxymethyl) pyrrolidine-2- carboxamido)ethyl) thiophen-3- yl)methylene)carbamate | *** | 2.07 | 742 |
| 250 | 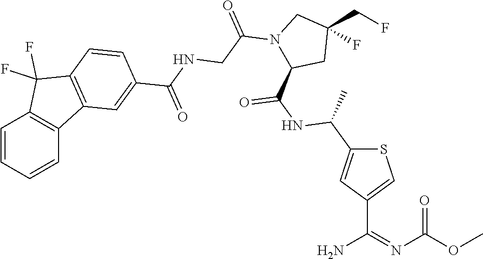 [SMILES: COC(=O)/N=C(/N)c1csc([C@@H](C)NC(=O)[C@@H]2C[C@](F)(CF)CN2C(=O)CNC(=O)c2ccc3c(c2)-c2ccccc2C3(F)F)c1] | (E)-methyl (amino(5- ((R)-1-((2S,4R)-1-((9,9- difluoro-9H-fluorene-3- carbonyl)glycyl)-4- fluoro-4- (fluoromethyl) pyrrolidine-2- carboxamido)ethyl) thiophen-3- yl)methylene)carbamate | *** | 1.43 | 660 |
| 251 | 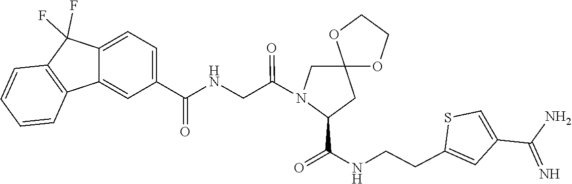 [SMILES: N=C(N)c1csc(CCNC(=O)[C@@H]2CC3(CN2C(=O)CNC(=O)c2ccc4c(c2)-c2ccccc2C4(F)F)OCCO3)c1] | (S)-N-(2-(4- carbamimidoylthiophen- 2-yl)ethyl)-7-((9,9- difluoro-9H-fluorene-3- carbonyl)glycyl)-1,4- dioxa-7- azaspiro[4.4]nonane-8- carboxamide | *** | 1.21 | 610 |
| 252 | 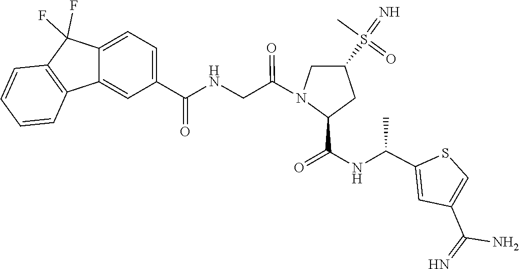 [SMILES: C[C@@H](NC(=O)[C@@H]1C[C@@H](S(C)(=N)=O)CN1C(=O)CNC(=O)c1ccc2c(c1)-c1ccccc1C2(F)F)c1cc(C(=N)N)cs1] | (2S,4R)-N-((R)-1-(4- carbamimidoylthiophen- 2-yl)ethyl)-1-((9,9- difluoro-9H-fluorene-3- carbonyl)glycyl)-4-((S)- S- methylsulfonimidoyl) pyrrolidine-2- carboxamide | *** | 1.08 | 629 |
| 253 | 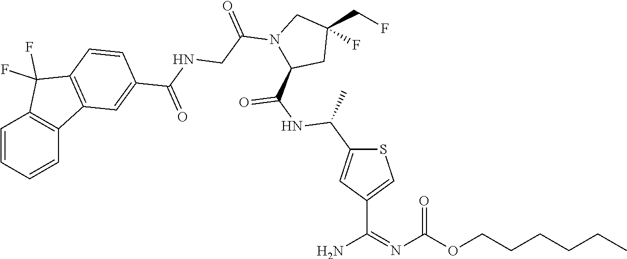 [SMILES: CCCCCCOC(=O)/N=C(/N)c1csc([C@@H](C)NC(=O)[C@@H]2C[C@](F)(CF)CN2C(=O)CNC(=O)c2ccc3c(c2)-c2ccccc2C3(F)F)c1] | (E)-hexyl (amino(5- ((R)-1-((2S,4R)-1-((9,9- difluoro-9H-fluorene-3- carbonyl)glycyl)-4- fluoro-4- (fluoromethyl) pyrrolidine-2- carboxamido)ethyl) thiophen-3- yl)methylene)carbamate | *** | 2.11 | 730 |
| 254 | 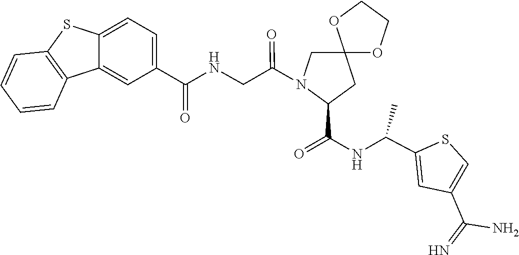 [SMILES: C[C@@H](NC(=O)[C@@H]1CC2(CN1C(=O)CNC(=O)c1ccc3sc4ccccc4c3c1)OCCO2)c1cc(C(=N)N)cs1] | (S)-N-((R)-1-(4- carbamimidoylthiophen- 2-yl)ethyl)-7- ((dibenzo[b,d]thiophene- 2-carbonyl)glycyl)-1,4- dioxa-7- azaspiro[4.4]nonane-8- carboxamide | *** | 1.24 | 592 |
| 255 | 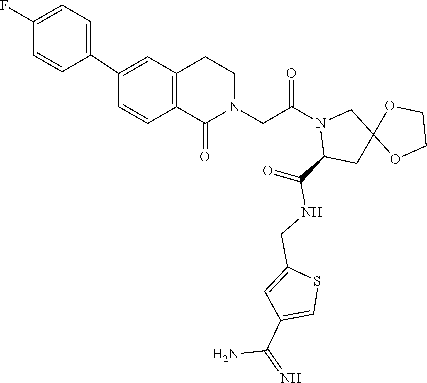 [SMILES: N=C(N)c1csc(CNC(=O)[C@@H]2CC3(CN2C(=O)CN2CCc4cc(-c5ccc(F)cc5)ccc4C2=O)OCCO3)c1] | (S)-N-((4- carbamimidoylthiophen- 2-yl)methyl)-7-(2-(6-(4- fluorophenyl)-1-oxo- 3,4-dihydroisoquinolin- 2(1H)-yl)acetyl)-1,4- dioxa-7- azaspiro[4.4]nonane-8- carboxamide | * | 1.26 | 592 |
| 256 | 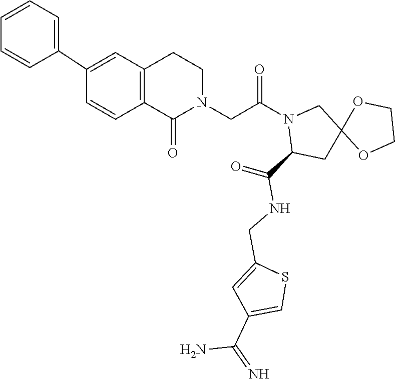 [SMILES: N=C(N)c1csc(CNC(=O)[C@@H]2CC3(CN2C(=O)CN2CCc4cc(-c5ccccc5)ccc4C2=O)OCCO3)c1] | (S)-N-((4- carbamimidoylthiophen- 2-yl)methyl)-7-(2-(1- oxo-6-phenyl-3,4- dihydroisoquinolin- 2(1H)-yl)acetyl)-1,4- dioxa-7- azaspiro[4.4]nonane-8- carboxamide | *** | 1.24 | 574 |
| 257 | 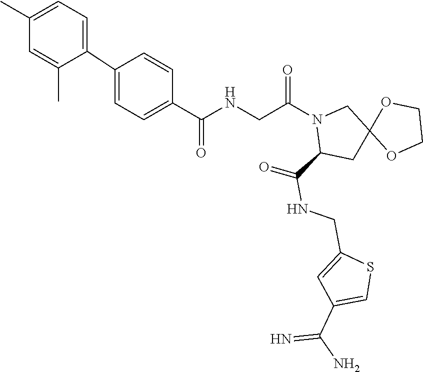 [SMILES: Cc1ccc(-c2ccc(C(=O)NCC(=O)N3CC4(C[C@H]3C(=O)NCc3cc(C(=N)N)cs3)OCCO4)cc2)c(C)c1] | (S)-N-((4- carbamimidoylthiophen- 2-yl)methyl)-7-((2′,4′- dimethyl-[1,1′- biphenyl]-4- carbonyl)glycyl)-1,4- dioxa-7- azaspiro[4.4]nonane-8- carboxamide | *** | 1.40 | 576 |
| 258 | 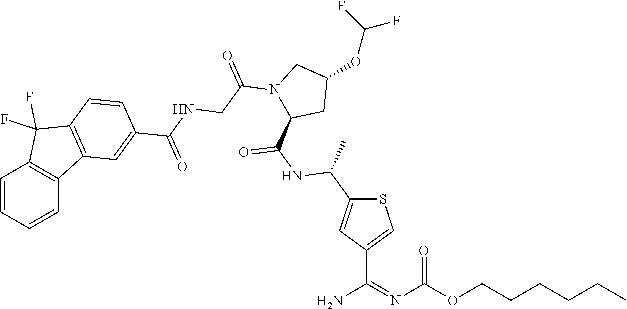 [SMILES: CCCCCCOC(=O)/N=C(/N)c1csc([C@@H](C)NC(=O)[C@@H]2C[C@@H](OC(F)F)CN2C(=O)CNC(=O)c2ccc3c(c2)-c2ccccc2C3(F)F)c1] | (E)-hexyl (amino(5- ((R)-1-((2S,4R)-1-((9,9- difluoro-9H-fluorene-3- carbonyl)glycyl)-4- (difluoromethoxy) pyrrolidine-2- carboxamido)ethyl) thiophen-3- yl)methylene)carbamate | *** | 2.16 | 746 |
| 259 | 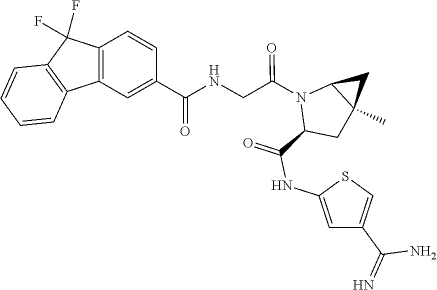 [SMILES: C[C@@]12C[C@@H]1N(C(=O)CNC(=O)c1ccc3c(c1)-c1ccccc1C3(F)F)[C@H](C(=O)Nc1cc(C(=N)N)cs1)C2] | (1S,3S,5S)-N-(4- carbamimidoylthiophen- 2-yl)-2-((9,9-difluoro- 9H-fluorene-3- carbonyl)glycyl)-5- methyl-2- azabicyclo[3.1.0]hexane- 3-carboxamide | * | 2.16 | 550 |
| 260 | 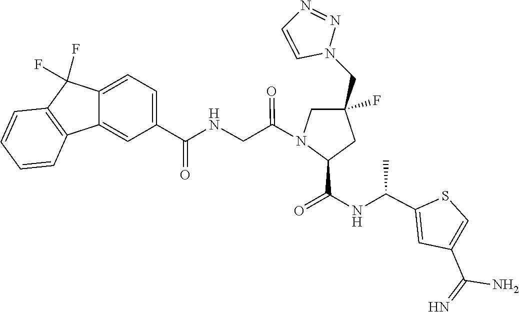 [SMILES: C[C@@H](NC(=O)[C@@H]1C[C@](F)(Cn2ccnn2)CN1C(=O)CNC(=O)c1ccc2c(c1)-c1ccccc1C2(F)F)c1cc(C(=N)N)cs1] | (2S,4R)-4-((1H-1,2,3- triazol-1-yl)methyl)-N- ((R)-1-(4- carbamimidoylthiophen- 2-yl)ethyl)-1-((9,9- difluoro-9H-fluorene-3- carbonyl)glycyl)-4- fluoropyrrolidine-2- carboxamide | *** | 1.19 | 651 |
| 261 | 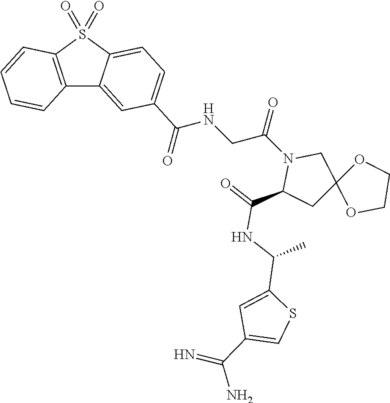 [SMILES: C[C@@H](NC(=O)[C@@H]1CC2(CN1C(=O)CNC(=O)c1ccc3c(c1)-c1ccccc1S3(=O)=O)OCCO2)c1cc(C(=N)N)cs1] | (S)-N-((R)-1-(4- carbamimidoylthiophen- 2-yl)ethyl)-7-((5,5- dioxidodibenzo[b,d] thiophene-2- carbonyl)glycyl)-1,4- dioxa-7- azaspiro[4.4]nonane-8- carboxamide | *** | 0.89 | 624 |
| 262 | 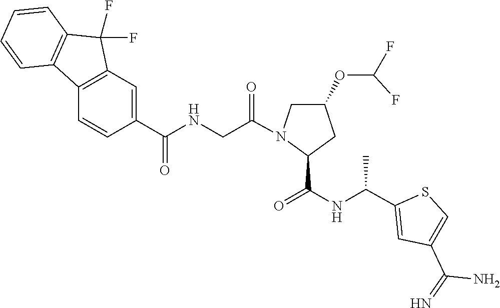 [SMILES: C[C@@H](NC(=O)[C@@H]1C[C@@H](OC(F)F)CN1C(=O)CNC(=O)c1ccc2c(c1)C(F)(F)c1ccccc1-2)c1cc(C(=N)N)cs1] | (2S,4R)-N-((R)-1-(4- carbamimidoylthiophen- 2-yl)ethyl)-1-((9,9- difluoro-9H-fluorene-2- carbonyl)glycyl)-4- (difluoromethoxy) pyrrolidine-2- carboxamide | *** | 1.33 | 618 |
| 263 | 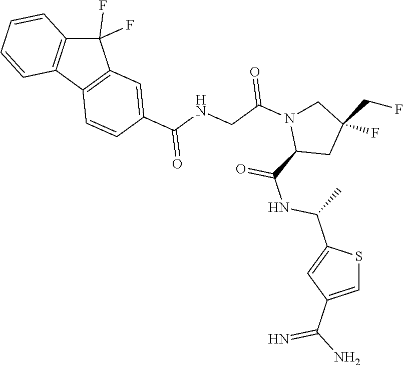 [SMILES: C[C@@H](NC(=O)[C@@H]1C[C@](F)(CF)CN1C(=O)CNC(=O)c1ccc2c(c1)C(F)(F)c1ccccc1-2)c1cc(C(=N)N)cs1] | (2S,4R)-N-((R)-1-(4- carbamimidoylthiophen- 2-yl)ethyl)-1-((9,9- difluoro-9H-fluorene-2- carbonyl)glycyl)-4- fluoro-4- (fluoromethyl)pyrrolidine- 2-carboxamide | *** | 1.27 | 602 |
| 264 | 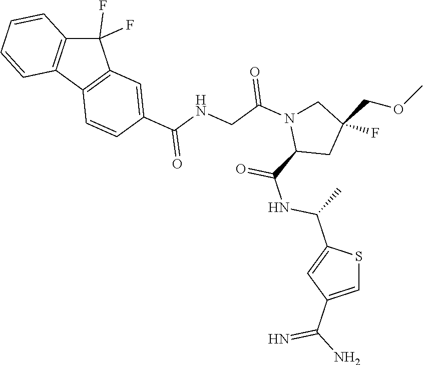 [SMILES: COC[C@@]1(F)C[C@@H](C(=O)N[C@H](C)c2cc(C(=N)N)cs2)N(C(=O)CNC(=O)c2ccc3c(c2)C(F)(F)c2ccccc2-3)C1] | (2S,4R)-N-((R)-1-(4- carbamimidoylthiophen- 2-yl)ethyl)-1-((9,9- difluoro-9H-fluorene-2- carbonyl)glycyl)-4- fluoro-4- (methoxymethyl) pyrrolidine-2- carboxamide | *** | 1.32 | 614 |
| 265 | 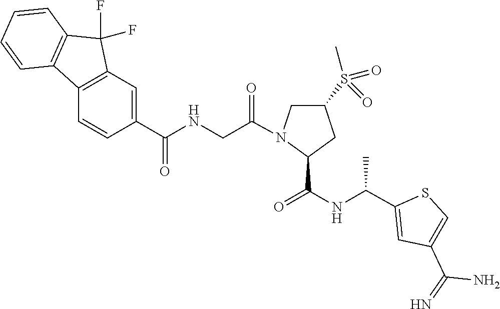 [SMILES: C[C@@H](NC(=O)[C@@H]1C[C@@H](S(C)(=O)=O)CN1C(=O)CNC(=O)c1ccc2c(c1)C(F)(F)c1ccccc1-2)c1cc(C(=N)N)cs1] | (2S,4R)-N-((R)-1-(4- carbamimidoylthiophen- 2-yl)ethyl)-1-((9,9- difluoro-9H-fluorene-2- carbonyl)glycyl)-4- (methylsulfonyl) pyrrolidine-2- carboxamide | *** | 1.18 | 630 |
| 266 | 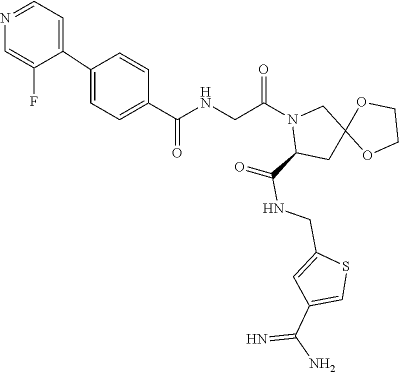 [SMILES: N=C(N)c1csc(CNC(=O)[C@@H]2CC3(CN2C(=O)CNC(=O)c2ccc(-c4ccncc4F)cc2)OCCO3)c1] | (S)-N-((4- carbamimidoylthiophen- 2-yl)methyl)-7-((4-(3- fluoropyridin-4- yl)benzoyl)glycyl)-1,4- dioxa-7- azaspiro[4.4]nonane-8- carboxamide | *** | 0.69 | 567 |
| 267 | 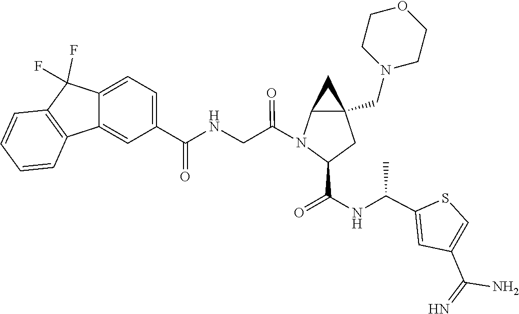 [SMILES: C[C@@H](NC(=O)[C@@H]1C[C@]2(CN3CCOCC3)C[C@@H]2N1C(=O)CNC(=O)c1ccc2c(c1)-c1ccccc1C2(F)F)c1cc(C(=N)N)cs1] | (1S,3S,5S)-N-((R)-1-(4- carbamimidoylthiophen- 2-yl)ethyl)-2-((9,9- difluoro-9H-fluorene-3- carbonyl)glycyl)-5- (morpholinomethyl)-2- azabicyclo[3.1.0]hexane- 3-carboxamide | *** | 0.78 | 663 |
| 268 | 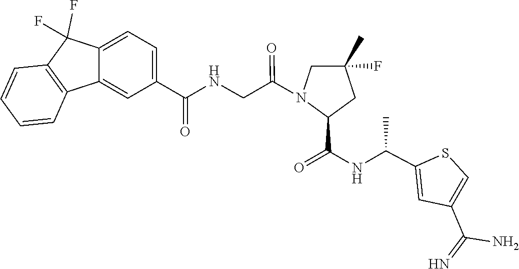 [SMILES: C[C@@H](NC(=O)[C@@H]1C[C@@](C)(F)CN1C(=O)CNC(=O)c1ccc2c(c1)-c1ccccc1C2(F)F)c1cc(C(=N)N)cs1] | (2S,4R)-N-((R)-1-(4- carbamimidoylthiophen- 2-yl)ethyl)-1-((9,9- difluoro-9H-fluorene-3- carbonyl)glycyl)-4- fluoro-4- methylpyrrolidine-2- carboxamide | *** | 1.30 | 584 |
| 269 | 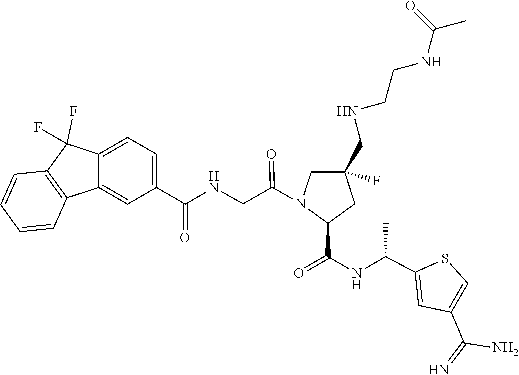 [SMILES: CC(=O)NCCNC[C@@]1(F)C[C@@H](C(=O)N[C@H](C)c2cc(C(=N)N)cs2)N(C(=O)CNC(=O)c2ccc3c(c2)-c2ccccc2C3(F)F)C1] | (2S,4S)-4-(((2- acetamidoethyl)amino) methyl)-N-((R)-1-(4- carbamimidoylthiophen- 2-yl)ethyl)-1-((9,9- difluoro-9H-fluorene-3- carbonyl)glycyl)-4- fluoropyrrolidine-2- carboxamide | *** | 0.83 | 684 |
| 270 | 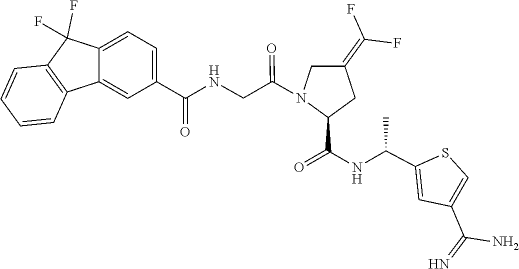 [SMILES: C[C@@H](NC(=O)[C@@H]1CC(=C(F)F)CN1C(=O)CNC(=O)c1ccc2c(c1)-c1ccccc1C2(F)F)c1cc(C(=N)N)cs1] | (S)-N-((R)-1-(4- carbamimidoylthiophen- 2-yl)ethyl)-1-((9,9- difluoro-9H-fluorene-3- carbonyl)glycyl)-4- (difluoromethylene) pyrrolidine-2- carboxamide | *** | 1.35 | 600 |
| 271 | 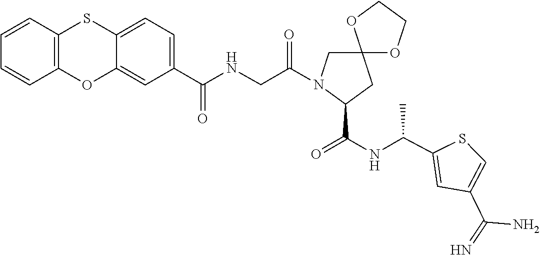 [SMILES: C[C@@H](NC(=O)[C@@H]1CC2(CN1C(=O)CNC(=O)c1ccc3c(c1)Oc1ccccc1S3)OCCO2)c1cc(C(=N)N)cs1] | (S)-N-((R)-1-(4- carbamimidoylthiophen- 2-yl)ethyl)-7- ((phenoxathiine-3- carbonyl)glycyl)-1,4- dioxa-7- azaspiro[4.4]nonane-8- carboxamide | *** | 1.23 | 608 |
| 272 | 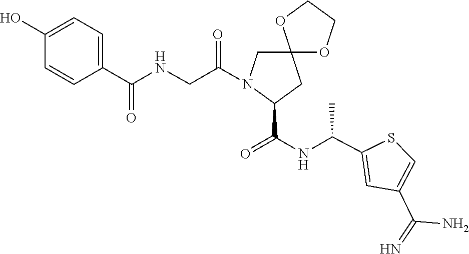 [SMILES: C[C@@H](NC(=O)[C@@H]1CC2(CN1C(=O)CNC(=O)c1ccc(O)cc1)OCCO2)c1cc(C(=N)N)cs1] | (S)-N-((R)-1-(4- carbamimidoylthiophen- 2-yl)ethyl)-7-((4- hydroxybenzoyl)glycyl)- 1,4-dioxa-7- azaspiro[4.4]nonane-8- carboxamide | *** | 0.26 | 502 |
| 273 | 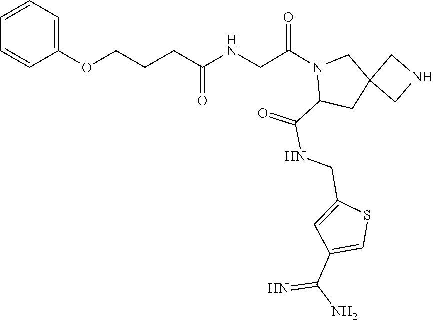 [SMILES: N=C(N)c1csc(CNC(=O)C2CC3(CNC3)CN2C(=O)CNC(=O)CCCOc2ccccc2)c1] | N-((4- carbamimidoylthiophen- 2-yl)methyl)-6-((4- phenoxybutanoyl)glycyl)- 2,6- diazaspiro[3.4]octane- 7-carboxamide | *** | 0.24 | 513 |
| 274 | 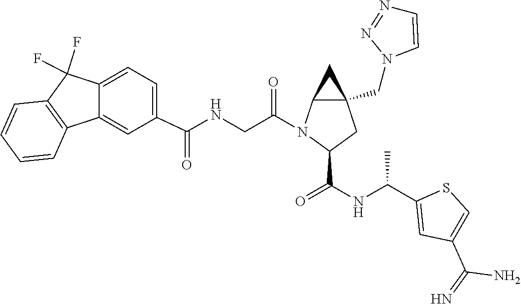 [SMILES: C[C@@H](NC(=O)[C@@H]1C[C@]2(Cn3ccnn3)C[C@@H]2N1C(=O)CNC(=O)c1ccc2c(c1)-c1ccccc1C2(F)F)c1cc(C(=N)N)cs1] | (1S,3S,5S)-5-((1H- 1,2,3-triazol-1- yl)methyl)-N-((R)-1-(4- carbamimidoylthiophen- 2-yl)ethyl)-2-((9,9- difluoro-9H-fluorene-3- carbonyl)glycyl)-2- azabicyclo[3.1.0]hexane- 3-carboxamide | *** | 1.18 | 645 |
| 275 | 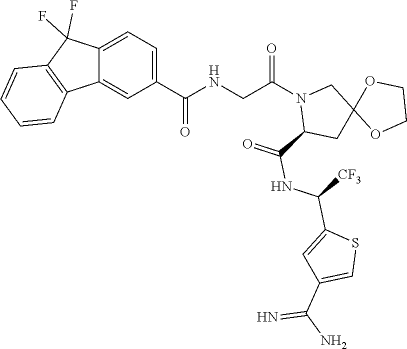 [SMILES: N=C(N)c1csc([C@@H](NC(=O)[C@@H]2CC3(CN2C(=O)CNC(=O)c2ccc4c(c2)-c2ccccc2C4(F)F)OCCO3)C(F)(F)F)c1] | (S)-N-((S)-1-(4- carbamimidoylthiophen- 2-yl)-2,2,2- trifluoroethyl)-7-((9,9- difluoro-9H-fluorene-3- carbonyl)glycyl)-1,4- dioxa-7- azaspiro[4.4]nonane-8- carboxamide | *** | 1.41 | 664 |
| 276 | 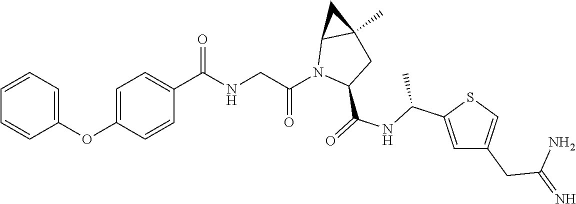 [SMILES: C[C@@H](NC(=O)[C@@H]1C[C@]2(C)C[C@@H]2N1C(=O)CNC(=O)c1ccc(Oc2ccccc2)cc1)c1cc(CC(=N)N)cs1] | (1S,3S,5S)-N-((R)-1-(4- (2-amino-2- iminoethyl)thiophen-2- yl)ethyl)-5-methyl-2-((4- phenoxybenzoyl)glycyl)- 2- azabicyclo[3.1.0]hexane- 3-carboxamide | *** | 1.28 | 560 |
| 277 | 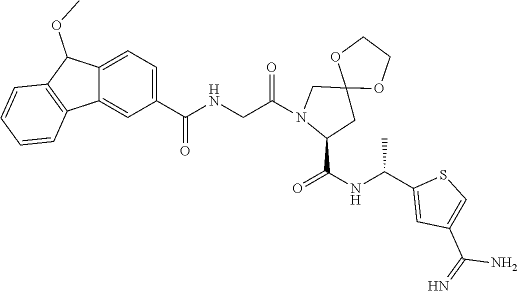 [SMILES: COC1c2ccccc2-c2cc(C(=O)NCC(=O)N3CC4(C[C@H]3C(=O)N[C@H](C)c3cc(C(=N)N)cs3)OCCO4)ccc21] | (8S)-N-((R)-1-(4- carbamimidoylthiophen- 2-yl)ethyl)-7-((9- methoxy-9H-fluorene-3- carbonyl)glycyl)-1,4- dioxa-7- azaspiro[4.4]nonane-8- carboxamide | *** | 1.04 | 604 |
| 278 | 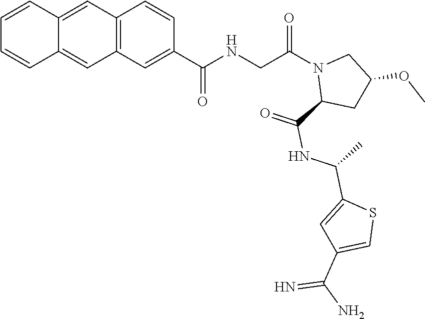 [SMILES: CO[C@@H]1C[C@@H](C(=O)N[C@H](C)c2cc(C(=N)N)cs2)N(C(=O)CNC(=O)c2ccc3cc4ccccc4cc3c2)C1] | (2S,4R)-1- ((anthracene-2- carbonyl)glycyl)-N-((R)- 1-(4- carbamimidoylthiophen- 2-yl)ethyl)-4- methoxypyrrolidine-2- carboxamide | *** | 1.20 | 558 |
| 279 | 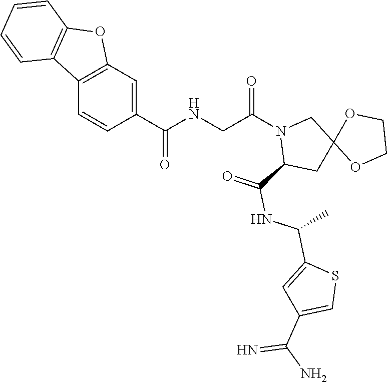 [SMILES: C[C@@H](NC(=O)[C@@H]1CC2(CN1C(=O)CNC(=O)c1ccc3c(c1)oc1ccccc13)OCCO2)c1cc(C(=N)N)cs1] | (S)-N-((R)-1-(4- carbamimidoylthiophen- 2-yl)ethyl)-7- ((dibenzo[b,d]furan-3- carbonyl)glycyl)-1,4- dioxa-7- azaspiro[4.4]nonane-8- carboxamide | *** | 1.08 | 576 |
| 280 | 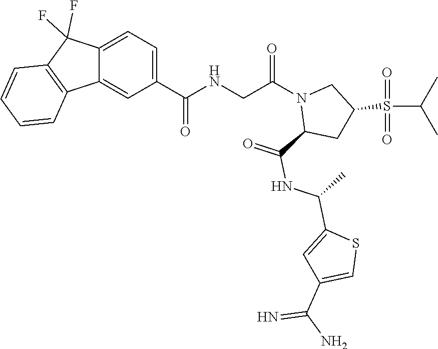 [SMILES: CC(C)S(=O)(=O)[C@@H]1C[C@@H](C(=O)N[C@H](C)c2cc(C(=N)N)cs2)N(C(=O)CNC(=O)c2ccc3c(c2)-c2ccccc2C3(F)F)C1] | (2S,4R)-N-((R)-1-(4- carbamimidoylthiophen- 2-yl)ethyl)-1-((9,9- difluoro-9H-fluorene-3- carbonyl)glycyl)-4- (isopropylsulfonyl) pyrrolidine-2- carboxamide | *** | 1.28 | 658 |
| 281 | 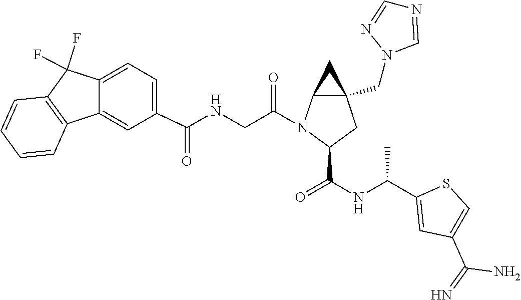 [SMILES: C[C@@H](NC(=O)[C@@H]1C[C@]2(Cn3cncn3)C[C@@H]2N1C(=O)CNC(=O)c1ccc2c(c1)-c1ccccc1C2(F)F)c1cc(C(=N)N)cs1] | (1S,3S,5S)-5-((1H- 1,2,4-triazol-1- yl)methyl)-N-((R)-1-(4- carbamimidoylthiophen- 2-yl)ethyl)-2-((9,9- difluoro-9H-fluorene-3- carbonyl)glycyl)-2- azabicyclo[3.1.0]hexane- 3-carboxamide | *** | 1.14 | 645 |
| 282 | 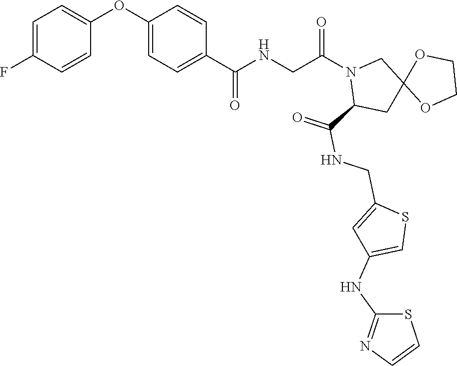 [SMILES: O=C(NCC(=O)N1CC2(C[C@H]1C(=O)NCc1cc(Nc3nccs3)cs1)OCCO2)c1ccc(Oc2ccc(F)cc2)cc1] | (S)-7-((4-(4- fluorophenoxy)benzoyl) glycyl)-N-((4-(thiazol-2- ylamino)thiophen-2- yl)methyl)-1,4-dioxa-7- azaspiro[4.4]nonane-8- carboxamide | * | 2.03 | 638 |
| 283 | 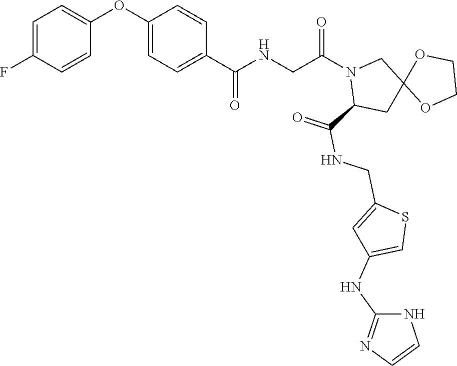 [SMILES: O=C(NCC(=O)N1CC2(C[C@H]1C(=O)NCc1cc(Nc3ncc[nH]3)cs1)OCCO2)c1ccc(Oc2ccc(F)cc2)cc1] | (S)-N-((4-((1H-imidazol- 2-yl)amino)thiophen-2- yl)methyl)-7-((4-(4- fluorophenoxy)benzoyl) glycyl)-1,4-dioxa-7- azaspiro[4.4]nonane-8- carboxamide | * | 1.22 | 621 |
| 284 | 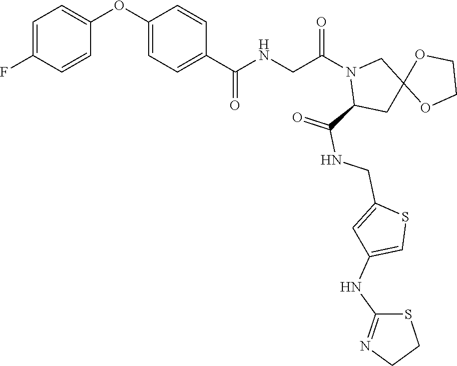 [SMILES: O=C(NCC(=O)N1CC2(C[C@H]1C(=O)NCc1cc(NC3=NCCS3)cs1)OCCO2)c1ccc(Oc2ccc(F)cc2)cc1] | (S)-N-((4-((4,5- dihydrothiazol-2- yl)amino)thiophen-2- yl)methyl)-7-((4-(4- fluorophenoxy)benzoyl) glycyl)-1,4-dioxa-7- azaspiro[4.4]nonane-8- carboxamide | * | 1.28 | 640 |
| 285 | 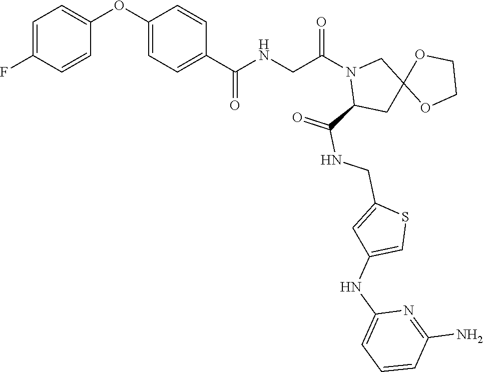 [SMILES: Nc1cccc(Nc2csc(CNC(=O)[C@@H]3CC4(CN3C(=O)CNC(=O)c3ccc(Oc5ccc(F)cc5)cc3)OCCO4)c2)n1] | (S)-N-((4-((6- aminopyridin-2- yl)amino)thiophen-2- yl)methyl)-7-((4-(4- fluorophenoxy)benzoyl) glycyl)-1,4-dioxa-7- azaspiro[4.4]nonane-8- carboxamide | * | 1.45 | 647 |
| 286 | 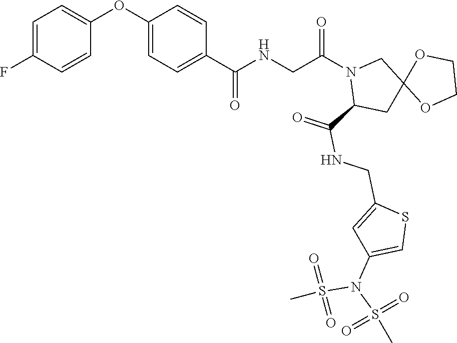 [SMILES: CS(=O)(=O)N(c1csc(CNC(=O)[C@@H]2CC3(CN2C(=O)CNC(=O)c2ccc(Oc4ccc(F)cc4)cc2)OCCO3)c1)S(C)(=O)=O] | (S)-7-((4-(4- fluorophenoxy)benzoyl) glycyl)-N-((4-((N- (methylsulfonyl)methyl) sulfonamido)thiophen- 2-yl)methyl)-1,4-dioxa- 7-azaspiro[4.4]nonane- 8-carboxamide | * | 2.00 | 711 |
| 287 | 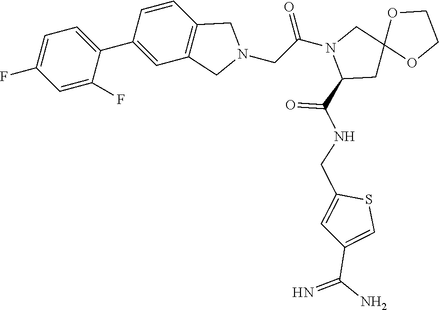 [SMILES: N=C(N)c1csc(CNC(=O)[C@@H]2CC3(CN2C(=O)CN2Cc4ccc(-c5ccc(F)cc5F)cc4C2)OCCO3)c1] | (S)-N-((4- carbamimidoylthiophen- 2-yl)methyl)-7-(2-(5- (2,4- difluorophenyl)isoindolin- 2-yl)acetyl)-1,4-dioxa- 7-azaspiro[4.4]nonane- 8-carboxamide | *** | 0.75 | 582 |
| 288 | 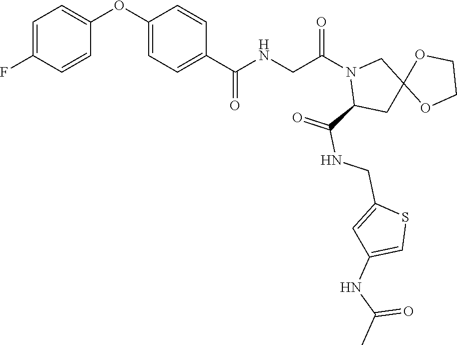 [SMILES: CC(=O)Nc1csc(CNC(=O)[C@@H]2CC3(CN2C(=O)CNC(=O)c2ccc(Oc4ccc(F)cc4)cc2)OCCO3)c1] | (S)-N-((4- acetamidothiophen-2- yl)methyl)-7-((4-(4- fluorophenoxy)benzoyl) glycyl)-1,4-dioxa-7- azaspiro[4.4]nonane-8- carboxamide | * | 1.75 | 597 |
| 289 | 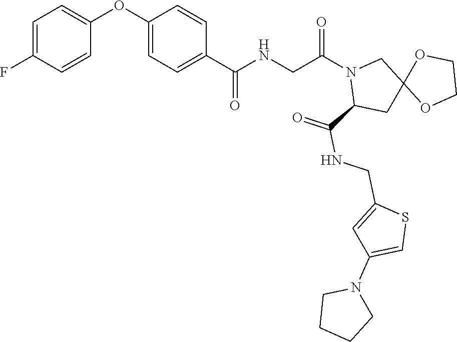 [SMILES: O=C(NCC(=O)N1CC2(C[C@H]1C(=O)NCc1cc(N3CCCC3)cs1)OCCO2)c1ccc(Oc2ccc(F)cc2)cc1] | (S)-7-((4-(4- fluorophenoxy)benzoyl) glycyl)-N-((4-(pyrrolidin- 1-yl)thiophen-2- yl)methyl)-1,4-dioxa-7- azaspiro[4.4]nonane-8- carboxamide | ** | 1.50 | 609 |
| 290 | 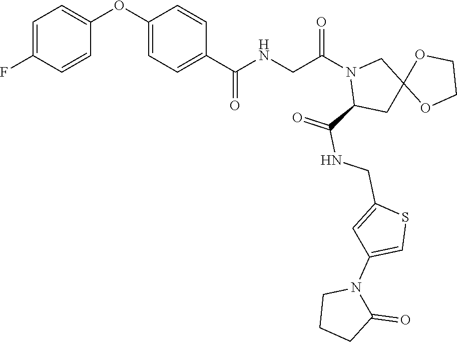 [SMILES: O=C(NCC(=O)N1CC2(C[C@H]1C(=O)NCc1cc(N3CCCC3=O)cs1)OCCO2)c1ccc(Oc2ccc(F)cc2)cc1] | (S)-7-((4-(4- fluorophenoxy)benzoyl) glycyl)-N-((4-(2- oxopyrrolidin-1- yl)thiophen-2- yl)methyl)-1,4-dioxa-7- azaspiro[4.4]nonane-8- carboxamide | * | 1.89 | 624 |
| 291 | 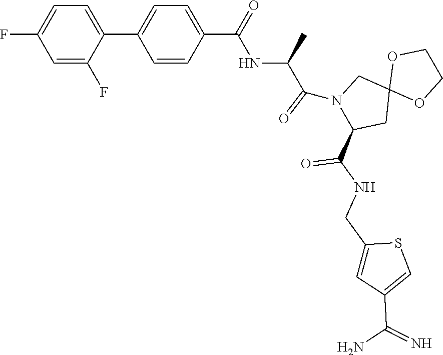 [SMILES: C[C@H](NC(=O)c1ccc(-c2ccc(F)cc2F)cc1)C(=O)N1CC2(C[C@H]1C(=O)NCc1cc(C(=N)N)cs1)OCCO2] | (S)-N-((4- carbamimidoylthiophen- 2-yl)methyl)-7-((2′,4′- difluoro-[1,1′-biphenyl]- 4-carbonyl)-L-alanyl)- 1,4-dioxa-7- azaspiro[4.4]nonane-8- carboxamide | *** | 1.24 | 598 |
| 292 | 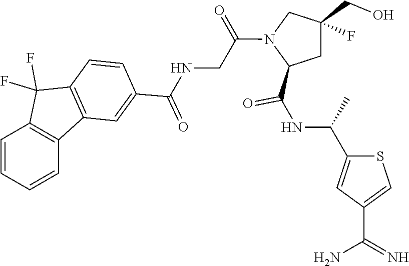 [SMILES: C[C@@H](NC(=O)[C@@H]1C[C@](F)(CO)CN1C(=O)CNC(=O)c1ccc2c(c1)-c1ccccc1C2(F)F)c1cc(C(=N)N)cs1] | (2S,4R)-N-((R)-1-(4- carbamimidoylthiophen- 2-yl)ethyl)-1-((9,9- difluoro-9H-fluorene-3- carbonyl)glycyl)-4- fluoro-4- (hydroxymethyl) pyrrolidine-2- carboxamide | *** | 1.11 | 600 |
| 293 | 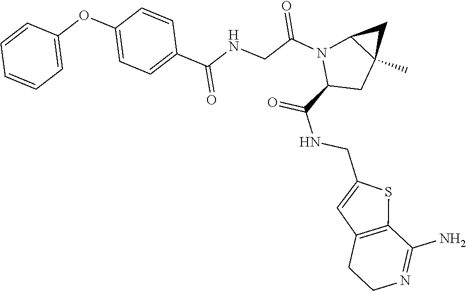 [SMILES: C[C@@]12C[C@@H]1N(C(=O)CNC(=O)c1ccc(Oc3ccccc3)cc1)[C@H](C(=O)NCc1cc3c(s1)C(N)=NCC3)C2] | (1S,3S,5S)-N-((7- amino-4,5- dihydrothieno[2,3- c]pyridin-2-yl)methyl)-5- methyl-2-((4- phenoxybenzoyl)glycyl)- 2- azabicyclo[3.1.0]hexane- 3-carboxamide | ** | 1.29 | 558 |
| 294 | 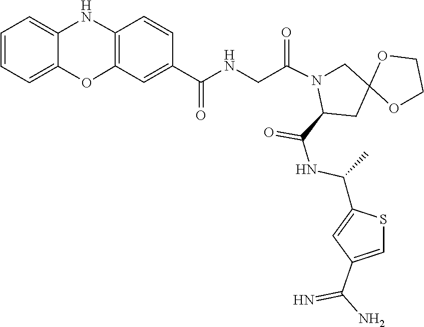 [SMILES: C[C@@H](NC(=O)[C@@H]1CC2(CN1C(=O)CNC(=O)c1ccc3c(c1)Oc1ccccc1N3)OCCO2)c1cc(C(=N)N)cs1] | N-(2-((S)-8-(((R)-1-(4- carbamimidoylthiophen- 2-yl)ethyl)carbamoyl)- 1,4-dioxa-7- azaspiro[4.4]nonan-7- yl)-2-oxoethyl)-10H- phenoxazine-3- carboxamide | *** | 0.97 | 591 |
| 295 | 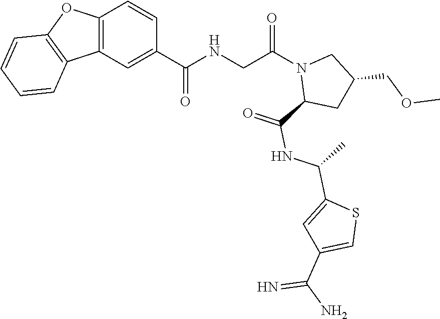 [SMILES: COC[C@@H]1C[C@@H](C(=O)N[C@H](C)c2cc(C(=N)N)cs2)N(C(=O)CNC(=O)c2ccc3oc4ccccc4c3c2)C1] | (2S,4R)-N-((R)-1-(4- carbamimidoylthiophen- 2-yl)ethyl)-1- ((dibenzo[b,d]furan-2- carbonyl)glycyl)-4- (methoxymethyl) pyrrolidine-2- carboxamide | *** | 1.12 | 562 |
| 296 | 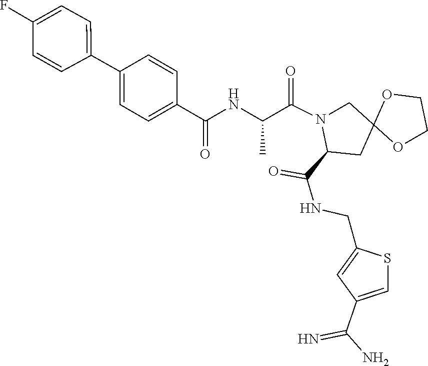 [SMILES: C[C@H](NC(=O)c1ccc(-c2ccc(F)cc2)cc1)C(=O)N1CC2(C[C@H]1C(=O)NCc1cc(C(=N)N)cs1)OCCO2] | (S)-N-((4- carbamimidoylthiophen- 2-yl)methyl)-7-((4′- fluoro-[1,1′-biphenyl]-4- carbonyl)-L-alanyl)-1,4- dioxa-7- azaspiro[4.4]nonane-8- carboxamide | *** | 1.27 | 580 |
| 297 | 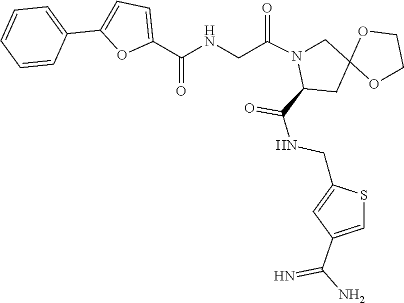 [SMILES: N=C(N)c1csc(CNC(=O)[C@@H]2CC3(CN2C(=O)CNC(=O)c2ccc(-c4ccccc4)o2)OCCO3)c1] | (S)-N-((4- carbamimidoylthiophen- 2-yl)methyl)-7-((5- phenylfuran-2- carbonyl)glycyl)-1,4- dioxa-7- azaspiro[4.4]nonane-8- carboxamide | *** | 0.99 | 538 |
| 298 | 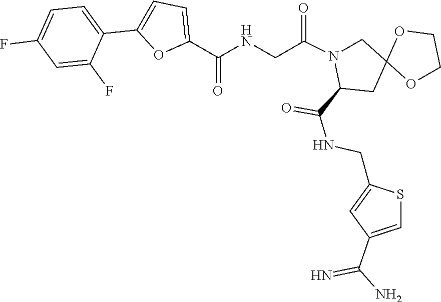 [SMILES: N=C(N)c1csc(CNC(=O)[C@@H]2CC3(CN2C(=O)CNC(=O)c2ccc(-c4ccc(F)cc4F)o2)OCCO3)c1] | (S)-N-((4- carbamimidoylthiophen- 2-yl)methyl)-7-((5-(2,4- difluorophenyl)furan-2- carbonyl)glycyl)-1,4- dioxa-7- azaspiro[4.4]nonane-8- carboxamide | *** | 1.13 | 574 |
| 299 | 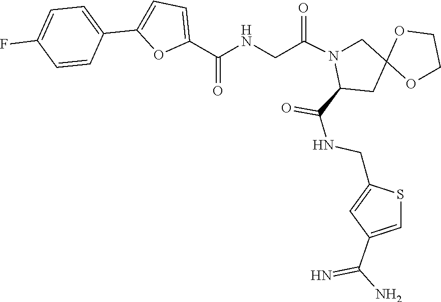 [SMILES: N=C(N)c1csc(CNC(=O)[C@@H]2CC3(CN2C(=O)CNC(=O)c2ccc(-c4ccc(F)cc4)o2)OCCO3)c1] | (S)-N-((4- carbamimidoylthiophen- 2-yl)methyl)-7-((5-(4- fluorophenyl)furan-2- carbonyl)glycyl)-1,4- dioxa-7- azaspiro[4.4]nonane-8- carboxamide | *** | 1.04 | 556 |
| 300 | 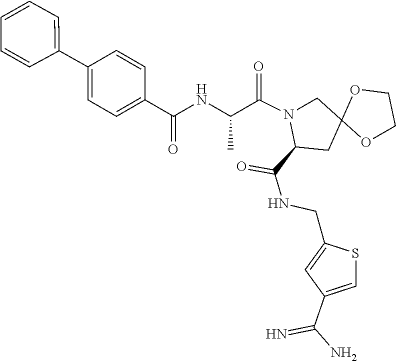 [SMILES: C[C@H](NC(=O)c1ccc(-c2ccccc2)cc1)C(=O)N1CC2(C[C@H]1C(=O)NCc1cc(C(=N)N)cs1)OCCO2] | (S)-7-(([1,1′-biphenyl]- 4-carbonyl)-L-alanyl)-N- ((4- carbamimidoylthiophen- 2-yl)methyl)-1,4-dioxa- 7-azaspiro[4.4]nonane- 8-carboxamide | *** | 1.24 | 562 |
| 301 | 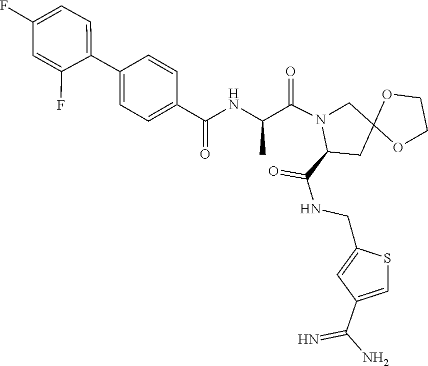 [SMILES: C[C@@H](NC(=O)c1ccc(-c2ccc(F)cc2F)cc1)C(=O)N1CC2(C[C@H]1C(=O)NCc1cc(C(=N)N)cs1)OCCO2] | (S)-N-((4- carbamimidoylthiophen- 2-yl)methyl)-7-((2′,4′- difluoro-[1,1′-biphenyl]- 4-carbonyl)-D-alanyl)- 1,4-dioxa-7- azaspiro[4.4]nonane-8- carboxamide | *** | 1.27 | 598 |
| 302 | 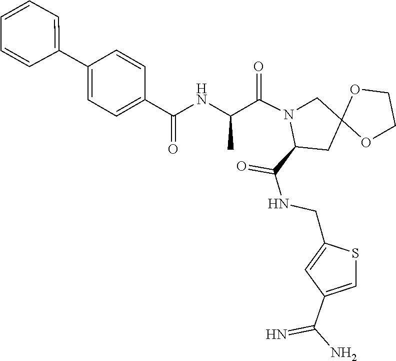 [SMILES: C[C@@H](NC(=O)c1ccc(-c2ccccc2)cc1)C(=O)N1CC2(C[C@H]1C(=O)NCc1cc(C(=N)N)cs1)OCCO2] | (S)-7-(([1,1′-biphenyl]- 4-carbonyl)-D-alanyl)- N-((4- carbamimidoylthiophen- 2-yl)methyl)-1,4-dioxa- 7-azaspiro[4.4]nonane- 8-carboxamide | *** | 1.21 | 562 |
| 303 | 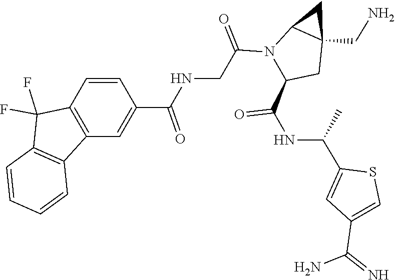 [SMILES: C[C@@H](NC(=O)[C@@H]1C[C@]2(CN)C[C@@H]2N1C(=O)CNC(=O)c1ccc2c(c1)-c1ccccc1C2(F)F)c1cc(C(=N)N)cs1] | (1S,3S,5S)-5- (aminomethyl)-N-((R)- 1-(4- carbamimidoylthiophen- 2-yl)ethyl)-2-((9,9- difluoro-9H-fluorene-3- carbonyl)glycyl)-2- azabicyclo[3.1.0]hexane- 3-carboxamide | *** | 0.92 | 593 |
| 304 | 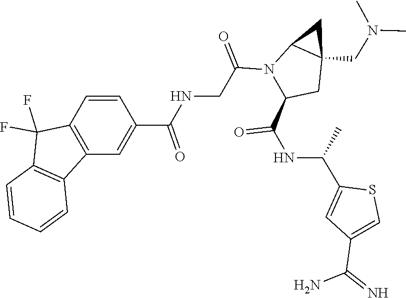 [SMILES: C[C@@H](NC(=O)[C@@H]1C[C@]2(CN(C)C)C[C@@H]2N1C(=O)CNC(=O)c1ccc2c(c1)-c1ccccc1C2(F)F)c1cc(C(=N)N)cs1] | (1S,3S,5S)-N-((R)-1-(4- carbamimidoylthiophen- 2-yl)ethyl)-2-((9,9- difluoro-9H-fluorene-3- carbonyl)glycyl)-5- ((dimethylamino)methyl)- 2- azabicyclo[3.1.0]hexane- 3-carboxamide | *** | 0.90 | 621 |
| 305 | 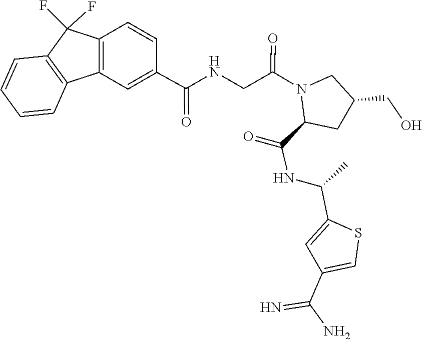 [SMILES: C[C@@H](NC(=O)[C@@H]1C[C@@H](CO)CN1C(=O)CNC(=O)c1ccc2c(c1)-c1ccccc1C2(F)F)c1cc(C(=N)N)cs1] | (2S,4R)-N-((R)-1-(4- carbamimidoylthiophen- 2-yl)ethyl)-1-((9,9- difluoro-9H-fluorene-3- carbonyl)glycyl)-4- (hydroxymethyl) pyrrolidine-2- carboxamide | *** | 1.24 | 582 |
| 306 | 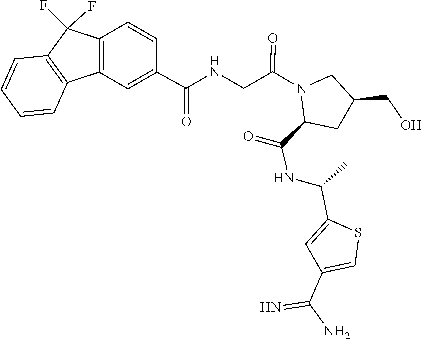 [SMILES: C[C@@H](NC(=O)[C@@H]1C[C@H](CO)CN1C(=O)CNC(=O)c1ccc2c(c1)-c1ccccc1C2(F)F)c1cc(C(=N)N)cs1] | (2S,4S)-N-((R)-1-(4- carbamimidoylthiophen- 2-yl)ethyl)-1-((9,9- difluoro-9H-fluorene-3- carbonyl)glycyl)-4- (hydroxymethyl) pyrrolidine-2- carboxamide | *** | 1.20 | 582 |
| 307 | 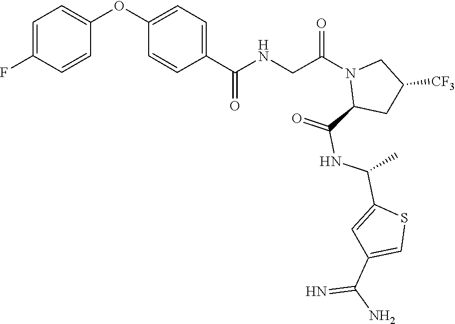 [SMILES: C[C@@H](NC(=O)[C@@H]1C[C@@H](C(F)(F)F)CN1C(=O)CNC(=O)c1ccc(Oc2ccc(F)cc2)cc1)c1cc(C(=N)N)cs1] | (2S,4R)-N-((R)-1-(4- carbamimidoylthiophen- 2-yl)ethyl)-1-((4-(4- fluorophenoxy)benzoyl) glycyl)-4- (trifluoromethyl) pyrrolidine-2- carboxamide | *** | 1.50 | 606 |
| 308 | 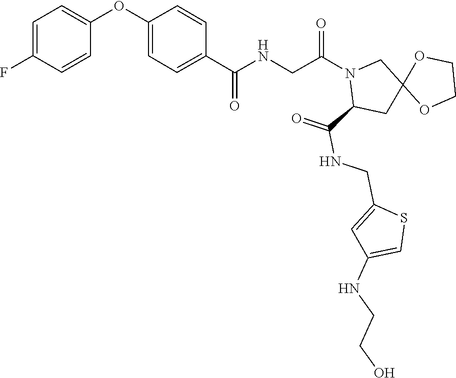 [SMILES: O=C(NCC(=O)N1CC2(C[C@H]1C(=O)NCc1cc(NCCO)cs1)OCCO2)c1ccc(Oc2ccc(F)cc2)cc1] | (S)-7-((4-(4- fluorophenoxy)benzoyl) glycyl)-N-((4-((2- hydroxyethyl)amino) thiophen-2-yl)methyl)-1,4- dioxa-7- azaspiro[4.4]nonane-8- carboxamide | * | 1.53 (A) | 599 |
| 309 | 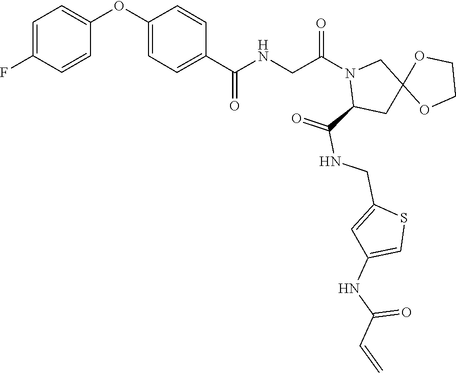 [SMILES: C=CC(=O)Nc1csc(CNC(=O)[C@@H]2CC3(CN2C(=O)CNC(=O)c2ccc(Oc4ccc(F)cc4)cc2)OCCO3)c1] | (S)-N-((4- acrylamidothiophen-2- yl)methyl)-7-((4-(4- fluorophenoxy)benzoyl) glycyl)-1,4-dioxa-7- azaspiro[4.4]nonane-8- carboxamide | * | 1.86 | 609 |
| 310 | 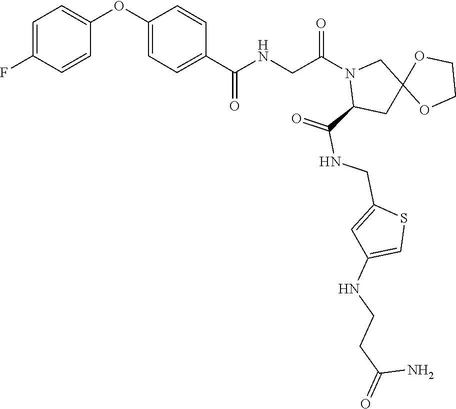 [SMILES: NC(=O)CCNc1csc(CNC(=O)[C@@H]2CC3(CN2C(=O)CNC(=O)c2ccc(Oc4ccc(F)cc4)cc2)OCCO3)c1] | (S)-N-((4-((3-amino-3- oxopropyl)amino) thiophen-2-yl) methyl)-7-((4- (4- fluorophenoxy)benzoyl) glycyl)-1,4-dioxa-7- azaspiro[4.4]nonane-8- carboxamide | * | 1.51 | 626 |
| 311 | 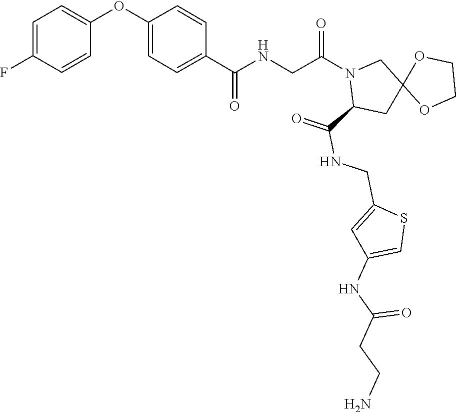 [SMILES: NCCC(=O)Nc1csc(CNC(=O)[C@@H]2CC3(CN2C(=O)CNC(=O)c2ccc(Oc4ccc(F)cc4)cc2)OCCO3)c1] | (S)-N-((4-(3- aminopropanamido) thiophen-2-yl) methyl)-7-((4- (4- fluorophenoxy)benzoyl) glycyl)-1,4-dioxa-7- azaspiro[4.4]nonane-8- carboxamide | * | 1.26 | 626 |
| 312 | 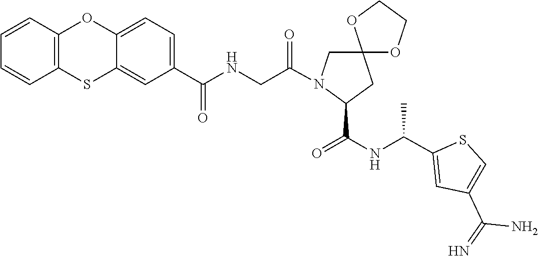 [SMILES: C[C@@H](NC(=O)[C@@H]1CC2(CN1C(=O)CNC(=O)c1ccc3c(c1)Sc1ccccc1O3)OCCO2)c1cc(C(=N)N)cs1] | (S)-N-((R)-1-(4- carbamimidoylthiophen- 2-yl)ethyl)-7- ((phenoxathiine-2- carbonyl)glycyl)-1,4- dioxa-7- azaspiro[4.4]nonane-8- carboxamide | *** | 1.30 | 608 |
| 313 | 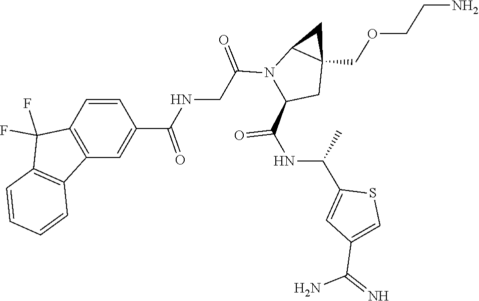 [SMILES: C[C@@H](NC(=O)[C@@H]1C[C@]2(COCCN)C[C@@H]2N1C(=O)CNC(=O)c1ccc2c(c1)-c1ccccc1C2(F)F)c1cc(C(=N)N)cs1] | (1S,3S,5R)-5-((2- aminoethoxy)methyl)- N-((R)-1-(4- carbamimidoylthiophen- 2-yl)ethyl)-2-((9,9- difluoro-9H-fluorene-3- carbonyl)glycyl)-2- azabicyclo[3.1.0]hexane- 3-carboxamide | *** | 0.87 | 637 |
| 314 | 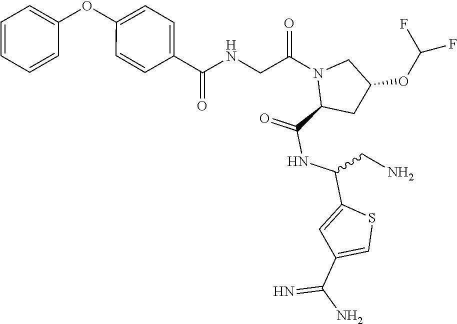 [SMILES: N=C(N)c1csc(C(CN)NC(=O)[C@@H]2C[C@@H](OC(F)F)CN2C(=O)CNC(=O)c2ccc(Oc3ccccc3)cc2)c1] | (2S,4R)-N-(2-amino-1- (4- carbamimidoylthiophen- 2-yl)ethyl)-4- (difluoromethoxy)-1-((4- phenoxybenzoyl)glycyl) pyrrolidine-2- carboxamide | * | 0.88 | 601 |
| 315 | 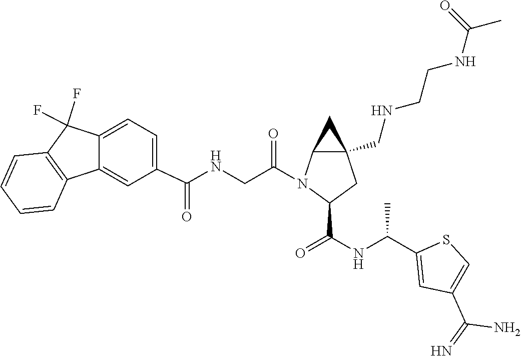 [SMILES: CC(=O)NCCNC[C@@]12C[C@@H]1N(C(=O)CNC(=O)c1ccc3c(c1)-c1ccccc1C3(F)F)[C@H](C(=O)N[C@H](C)c1cc(C(=N)N)cs1)C2] | (1S,3S,5S)-5-(((2- acetamidoethyl)amino) methyl)-N-((R)-1-(4- carbamimidoylthiophen- 2-yl)ethyl)-2-((9,9- difluoro-9H-fluorene-3- carbonyl)glycyl)-2- azabicyclo[3.1.0]hexane- 3-carboxamide | *** | 0.84 | 678 |
| 316 | 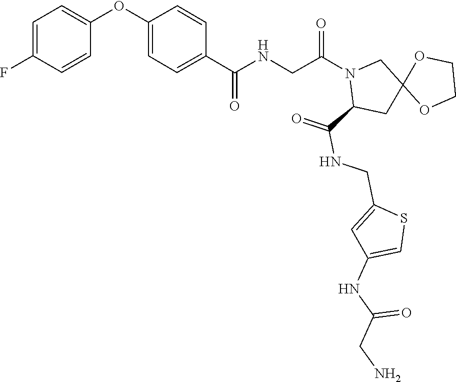 [SMILES: NCC(=O)Nc1csc(CNC(=O)[C@@H]2CC3(CN2C(=O)CNC(=O)c2ccc(Oc4ccc(F)cc4)cc2)OCCO3)c1] | (S)-N-((4-(2- aminoacetamido) thiophen-2-yl) methyl)-7-((4- (4- fluorophenoxy)benzoyl) glycyl)-1,4-dioxa-7- azaspiro[4.4]nonane-8- carboxamide | ** | 1.33 | 612 |
| 317 | 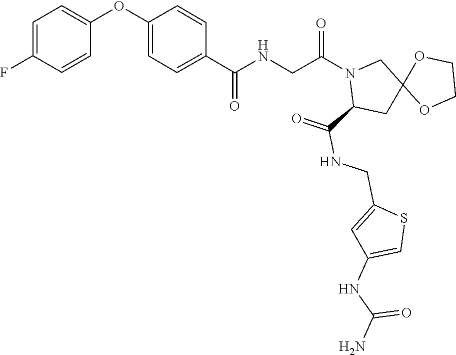 [SMILES: NC(=O)Nc1csc(CNC(=O)[C@@H]2CC3(CN2C(=O)CNC(=O)c2ccc(Oc4ccc(F)cc4)cc2)OCCO3)c1] | (S)-7-((4-(4- fluorophenoxy)benzoyl) glycyl)-N-((4- ureidothiophen-2- yl)methyl)-1,4-dioxa-7- azaspiro[4.4]nonane-8- carboxamide | * | 1.62 | 598 |
| 318 | 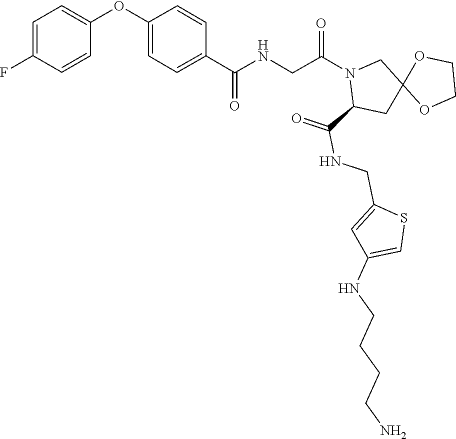 [SMILES: NCCCCNc1csc(CNC(=O)[C@@H]2CC3(CN2C(=O)CNC(=O)c2ccc(Oc4ccc(F)cc4)cc2)OCCO3)c1] | (S)-N-((4-((4- aminobutyl)amino) thiophen-2-yl) methyl)-7-((4- (4- fluorophenoxy)benzoyl) glycyl)-1,4-dioxa-7- azaspiro[4.4]nonane-8- carboxamide | * | 1.31 | 626 |
| 319 | 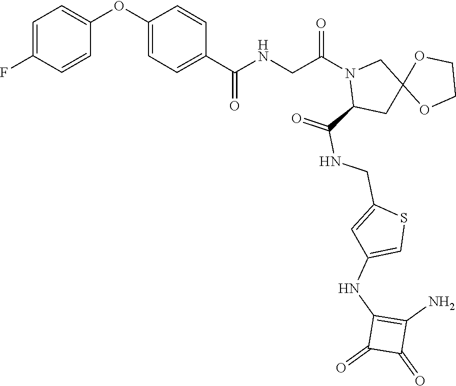 [SMILES: Nc1c(Nc2csc(CNC(=O)[C@@H]3CC4(CN3C(=O)CNC(=O)c3ccc(Oc5ccc(F)cc5)cc3)OCCO4)c2)c(=O)c1=O] | (S)-N-((4-((2-amino- 3,4-dioxocyclobut-1-en- 1-yl)amino)thiophen-2- yl)methyl)-7-((4-(4- fluorophenoxy)benzoyl) glycyl)-1,4-dioxa-7- azaspiro[4.4]nonane-8- carboxamide | * | 1.62 | 650 |
| 320 | 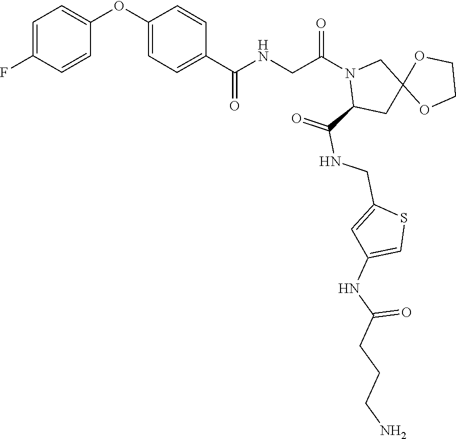 [SMILES: NCCCC(=O)Nc1csc(CNC(=O)[C@@H]2CC3(CN2C(=O)CNC(=O)c2ccc(Oc4ccc(F)cc4)cc2)OCCO3)c1] | (S)-N-((4-(4- aminobutanamido) thiophen-2-yl) methyl)-7-((4- (4- fluorophenoxy)benzoyl) glycyl)-1,4-dioxa-7- azaspiro[4.4]nonane-8- carboxamide | * | 1.34 | 640 |
| 321 | 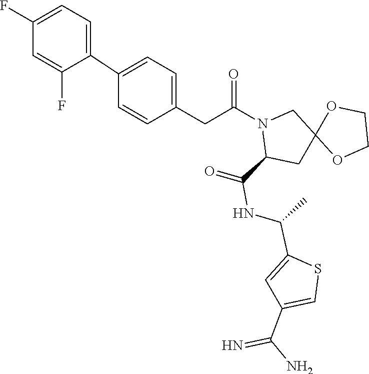 [SMILES: C[C@@H](NC(=O)[C@@H]1CC2(CN1C(=O)Cc1ccc(-c3ccc(F)cc3F)cc1)OCCO2)c1cc(C(=N)N)cs1] | (S)-N-((R)-1-(4- carbamimidoylthiophen- 2-yl)ethyl)-7-(2-(2′,4′- difluoro-[1,1′-biphenyl]- 4-yl)acetyl)-1,4-dioxa-7- azaspiro[4.4]nonane-8- carboxamide | *** | 1.51 | 555 |
| 322 | 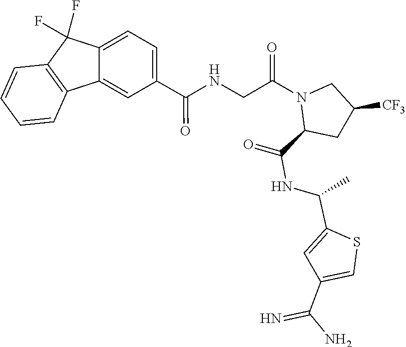 [SMILES: C[C@@H](NC(=O)[C@@H]1C[C@H](C(F)(F)F)CN1C(=O)CNC(=O)c1ccc2c(c1)-c1ccccc1C2(F)F)c1cc(C(=N)N)cs1] | (2S,4S)-N-((R)-1-(4- carbamimidoylthiophen- 2-yl)ethyl)-1-((9,9- difluoro-9H-fluorene-3- carbonyl)glycyl)-4- (trifluoromethyl) pyrrolidine-2- carboxamide | *** | 1.47 | 620 |
| 323 | 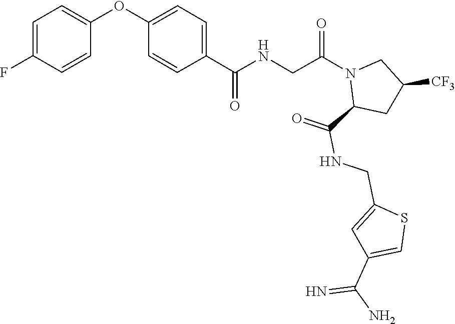 [SMILES: N=C(N)c1csc(CNC(=O)[C@@H]2C[C@H](C(F)(F)F)CN2C(=O)CNC(=O)c2ccc(Oc3ccc(F)cc3)cc2)c1] | (2S,4S)-N-((R)-1-(4- carbamimidoylthiophen- 2-yl)ethyl)-1-((4-(4- fluorophenoxy)benzoyl) glycyl)-4- (trifluoromethyl) pyrrolidine-2- carboxamide | *** | 1.43 | 606 |
| 324 | 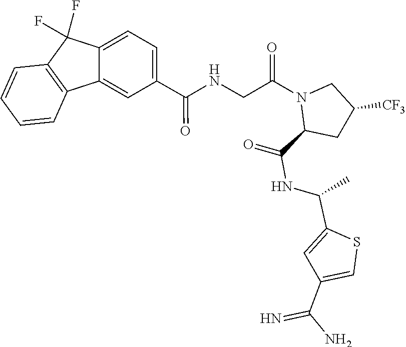 [SMILES: C[C@@H](NC(=O)[C@@H]1C[C@@H](C(F)(F)F)CN1C(=O)CNC(=O)c1ccc2c(c1)-c1ccccc1C2(F)F)c1cc(C(=N)N)cs1] | (2S,4R)-N-((R)-1-(4- carbamimidoylthiophen- 2-yl)ethyl)-1-((9,9- difluoro-9H-fluorene-3- carbonyl)glycyl)-4- (trifluoromethyl) pyrrolidine-2- carboxamide | *** | 1.50 | 620 |
| 325 | 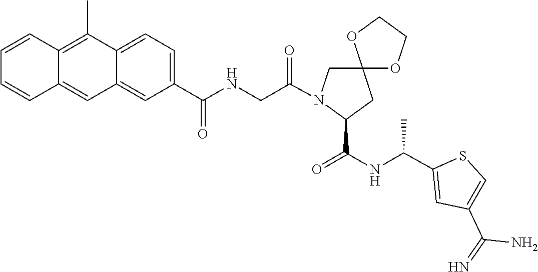 [SMILES: Cc1c2ccccc2cc2cc(C(=O)NCC(=O)N3CC4(C[C@H]3C(=O)N[C@H](C)c3cc(C(=N)N)cs3)OCCO4)ccc12] | (S)-N-((R)-1-(4- carbamimidoylthiophen- 2-yl)ethyl)-7-((10- methylanthracene-2- carbonyl)glycyl)-1,4- dioxa-7- azaspiro[4.4]nonane-8- carboxamide | *** | 1.39 | 600 |
| 326 | 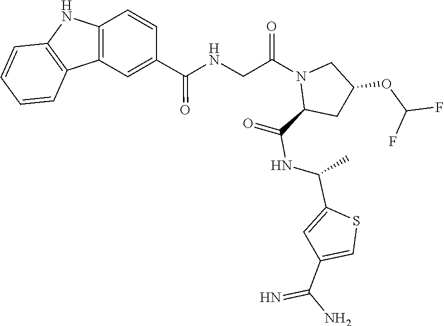 [SMILES: C[C@@H](NC(=O)[C@@H]1C[C@@H](OC(F)F)CN1C(=O)CNC(=O)c1ccc2[nH]c3ccccc3c2c1)c1cc(C(=N)N)cs1] | N-(2-((2S,4R)-2-(((R)-1- (4- carbamimidoylthiophen- 2-yl)ethyl)carbamoyl)- 4- (difluoromethoxy) pyrrolidin-1-yl)- 2-oxoethyl)- 9H-carbazole-3- carboxamide | *** | 1.10 | 583 |
| 327 | 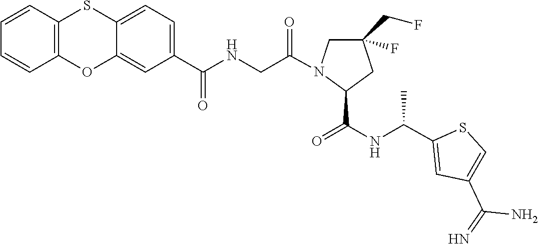 [SMILES: C[C@@H](NC(=O)[C@@H]1C[C@](F)(CF)CN1C(=O)CNC(=O)c1ccc2c(c1)Oc1ccccc1S2)c1cc(C(=N)N)cs1] | (2S,4R)-N-((R)-1-(4- carbamimidoylthiophen- 2-yl)ethyl)-4-fluoro-4- (fluoromethyl)-1- ((phenoxathiine-3- carbonyl)glycyl) pyrrolidine-2- carboxamide | *** | 1.34 | 601 |
| 328 | 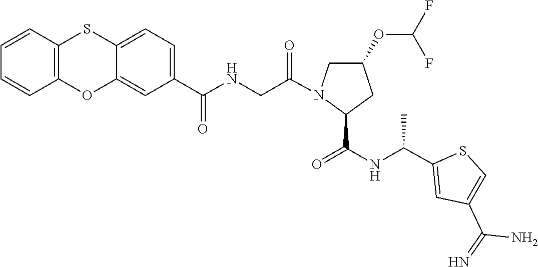 [SMILES: C[C@@H](NC(=O)[C@@H]1C[C@@H](OC(F)F)CN1C(=O)CNC(=O)c1ccc2c(c1)Oc1ccccc1S2)c1cc(C(=N)N)cs1] | (2S,4R)-N-((R)-1-(4- carbamimidoylthiophen- 2-yl)ethyl)-4- (difluoromethoxy)-1- ((phenoxathiine-3- carbonyl)glycyl) pyrrolidine-2- carboxamide | *** | 1.41 | 617 |
| 329 | 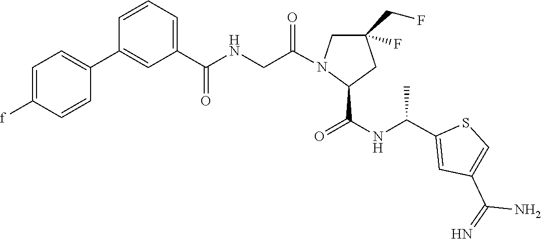 | (2S,4R)-N-((R)-1-(4- carbamimidoylthiophen- 2-yl)ethyl)-4-fluoro-1- ((4′-fluoro-[1,1′- biphenyl]-3- carbonyl)glycyl)-4- (fluoromethyl)pyrrolidine- 2-carboxamide | *** | 1.30 | 572 |
| 330 | 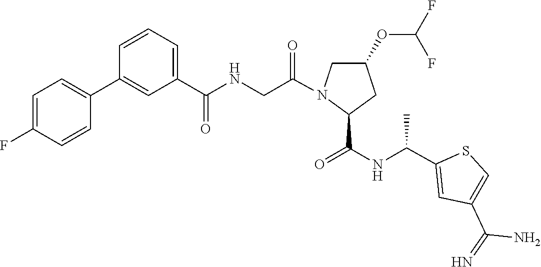 [SMILES: C[C@@H](NC(=O)[C@@H]1C[C@@H](OC(F)F)CN1C(=O)CNC(=O)c1cccc(-c2ccc(F)cc2)c1)c1cc(C(=N)N)cs1] | (2S,4R)-N-((R)-1-(4- carbamimidoylthiophen- 2-yl)ethyl)-4- (difluoromethoxy)-1- ((4′-fluoro-[1,1′- biphenyl]-3- carbonyl)glycyl) pyrrolidine-2- carboxamide | *** | 1.30 | 588 |
| 331 | 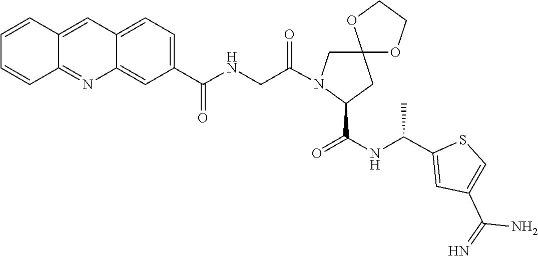 [SMILES: C[C@@H](NC(=O)[C@@H]1CC2(CN1C(=O)CNC(=O)c1ccc3cc4ccccc4nc3c1)OCCO2)c1cc(C(=N)N)cs1] | (S)-7-((acridine-3- carbonyl)glycyl)-N-((R)- 1-(4- carbamimidoylthiophen- 2-yl)ethyl)-1,4-dioxa-7- azaspiro[4.4]nonane-8- carboxamide | *** | 0.90 | 587 |
| 332 | 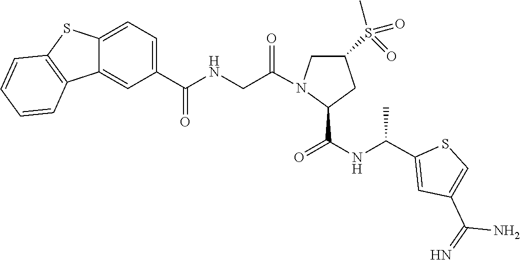 [SMILES: C[C@@H](NC(=O)[C@@H]1C[C@@H](S(C)(=O)=O)CN1C(=O)CNC(=O)c1ccc2sc3ccccc3c2c1)c1cc(C(=N)N)cs1] | (2S,4R)-N-((R)-1-(4- carbamimidoylthiophen- 2-yl)ethyl)-1- ((dibenzo[b,d]thiophene- 2-carbonyl)glycyl)-4- (methylsulfonyl) pyrrolidine-2- carboxamide | *** | 1.13 | 612 |
| 333 | 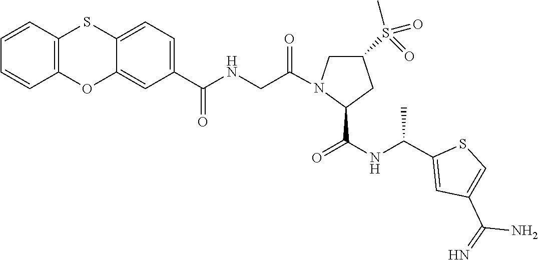 [SMILES: C[C@@H](NC(=O)[C@@H]1C[C@@H](S(C)(=O)=O)CN1C(=O)CNC(=O)c1ccc2c(c1)Oc1ccccc1S2)c1cc(C(=N)N)cs1] | (2S,4R)-N-((R)-1-(4- carbamimidoylthiophen- 2-yl)ethyl)-4- (methylsulfonyl)-1- ((phenoxathiine-3- carbonyl)glycyl) pyrrolidine-2- carboxamide | *** | 1.16 | 628 |
| 334 | 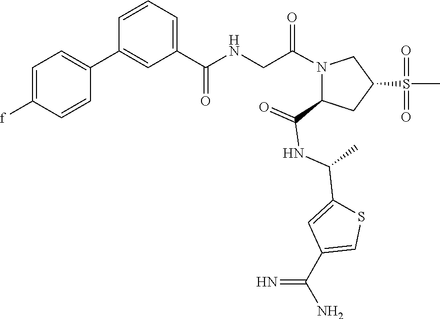 | (2S,4R)-N-((R)-1-(4- carbamimidoylthiophen- 2-yl)ethyl)-1-((4′-fluoro- [1,1′-biphenyl]-3- carbonyl)glycyl)-4- (methylsulfonyl) pyrrolidine-2- carboxamide | *** | 1.08 | 600 |
| 335 | 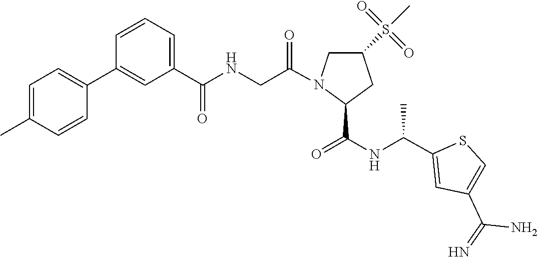 [SMILES: Cc1ccc(-c2cccc(C(=O)NCC(=O)N3C[C@H](S(C)(=O)=O)C[C@H]3C(=O)N[C@H](C)c3cc(C(=N)N)cs3)c2)cc1] | (2S,4R)-N-((R)-1-(4- carbamimidoylthiophen- 2-yl)ethyl)-1-((4′- methyl-[1,1′-biphenyl]- 3-carbonyl)glycyl)-4- (methylsulfonyl) pyrrolidine-2- carboxamide | *** | 1.15 | 596 |
| 336 | 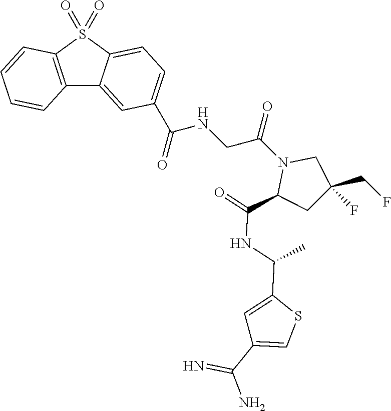 [SMILES: C[C@@H](NC(=O)[C@@H]1C[C@](F)(CF)CN1C(=O)CNC(=O)c1ccc2c(c1)-c1ccccc1S2(=O)=O)c1cc(C(=N)N)cs1] | (2S,4R)-N-((R)-1-(4- carbamimidoylthiophen- 2-yl)ethyl)-1-((5,5- dioxidodibenzo[b,d] thiophene-2- carbonyl)glycyl)-4- fluoro-4- (fluoromethyl)pyrrolidine- 2-carboxamide | *** | 0.92 | 616 |
| 337 | 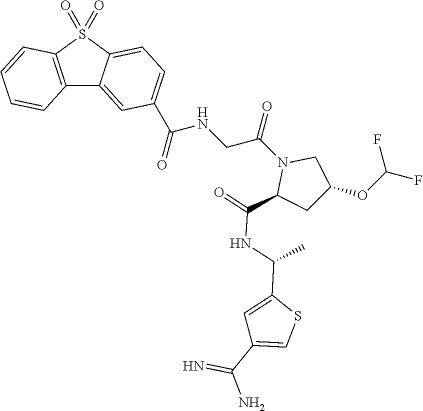 [SMILES: C[C@@H](NC(=O)[C@@H]1C[C@@H](OC(F)F)CN1C(=O)CNC(=O)c1ccc2c(c1)-c1ccccc1S2(=O)=O)c1cc(C(=N)N)cs1] | (2S,4R)-N-((R)-1-(4- carbamimidoylthiophen- 2-yl)ethyl)-4- (difluoromethoxy)-1- ((5,5- dioxidodibenzo[b,d] thiophene-2- carbonyl)glycyl) pyrrolidine-2- carboxamide | *** | 0.97 | 632 |
| 338 | 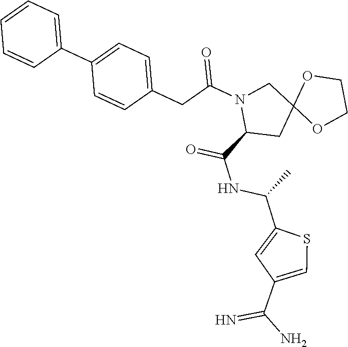 [SMILES: C[C@@H](NC(=O)[C@@H]1CC2(CN1C(=O)Cc1ccc(-c3ccccc3)cc1)OCCO2)c1cc(C(=N)N)cs1] | (S)-7-(2-([1,1′- biphenyl]-4-yl)acetyl)- N-((R)-1-(4- carbamimidoylthiophen- 2-yl)ethyl)-1,4-dioxa-7- azaspiro[4.4]nonane-8- carboxamide | *** | 1.42 | 519 |
| 339 | 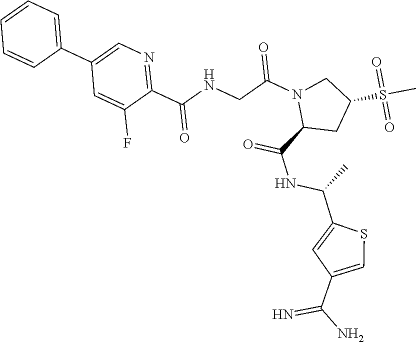 [SMILES: C[C@@H](NC(=O)[C@@H]1C[C@@H](S(C)(=O)=O)CN1C(=O)CNC(=O)c1ncc(-c2ccccc2)cc1F)c1cc(C(=N)N)cs1] | N-(2-((2,4R)-2-(((R)-1- (4- carbamimidoylthiophen- 2-yl)ethyl)carbamoyl)- 4- (methylsulfonyl) pyrrolidin-1-yl)- 2-oxoethyl)-3- fluoro-5- phenylpicolinamide | *** | 0.94 | 601 |
| 340 | 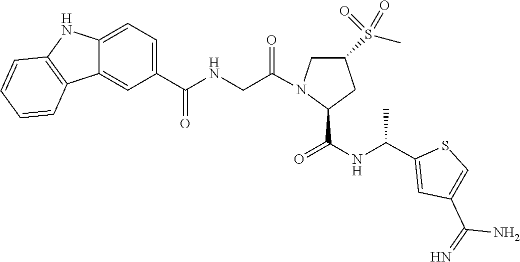 [SMILES: C[C@@H](NC(=O)[C@@H]1C[C@@H](S(C)(=O)=O)CN1C(=O)CNC(=O)c1ccc2[nH]c3ccccc3c2c1)c1cc(C(=N)N)cs1] | N-(2-((2S,4R)-2-(((R)-1- (4- carbamimidoylthiophen- 2-yl)ethyl)carbamoyl)- 4- (methylsulfonyl) pyrrolidin-1-yl)- 2-oxoethyl)-9H- carbazole-3- carboxamide | *** | 0.82 | 595 |
| 341 | 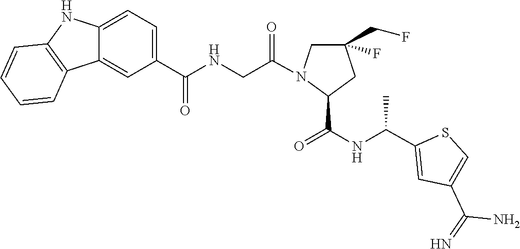 [SMILES: C[C@@H](NC(=O)[C@@H]1C[C@](F)(CF)CN1C(=O)CNC(=O)c1ccc2[nH]c3ccccc3c2c1)c1cc(C(=N)N)cs1] | N-(2-((2S,4R)-2-(((R)-1- (4- carbamimidoylthiophen- 2-yl)ethyl)carbamoyl)- 4-fluoro-4- (fluoromethyl)pyrrolidin- 1-yl)-2-oxoethyl)-9H- carbazole-3- carboxamide | *** | 1.03 | 567 |
| 342 | 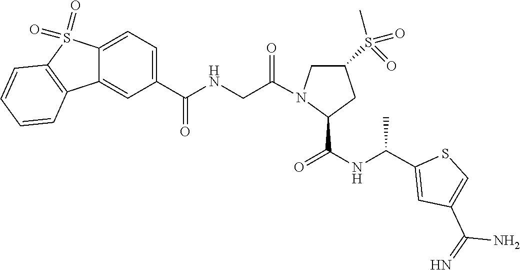 [SMILES: C[C@@H](NC(=O)[C@@H]1C[C@@H](S(C)(=O)=O)CN1C(=O)CNC(=O)c1ccc2c(c1)-c1ccccc1S2(=O)=O)c1cc(C(=N)N)cs1] | (2S,4R)-N-((R)-1-(4- carbamimidoylthiophen- 2-yl)ethyl)-1-((5,5- dioxidodibenzo[b,d] thiophene-2- carbonyl)glycyl)-4- (methylsulfonyl) pyrrolidine-2- carboxamide | *** | 0.73 | 644 |
| 343 | 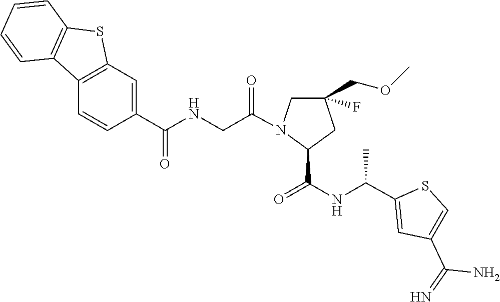 [SMILES: COC[C@@]1(F)C[C@@H](C(=O)N[C@H](C)c2cc(C(=N)N)cs2)N(C(=O)CNC(=O)c2ccc3c(c2)sc2ccccc23)C1] | (2S,4R)-N-((R)-1-(4- carbamimidoylthiophen- 2-yl)ethyl)-1- ((dibenzo[b,d]thiophene- 3-carbonyl)glycyl)-4- fluoro-4- (methoxymethyl) pyrrolidine-2- carboxamide | *** | 1.23 | 596 |
| 344 | 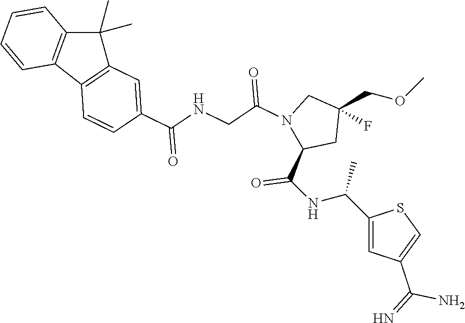 [SMILES: COC[C@@]1(F)C[C@@H](C(=O)N[C@H](C)c2cc(C(=N)N)cs2)N(C(=O)CNC(=O)c2ccc3c(c2)C(C)(C)c2ccccc2-3)C1] | (2S,4R)-N-((R)-1-(4- carbamimidoylthiophen- 2-yl)ethyl)-1-((9,9- dimethyl-9H-fluorene-2- carbonyl)glycyl)-4- fluoro-4- (methoxymethyl) pyrrolidine-2- carboxamide | *** | 1.39 | 606 |
| 345 | 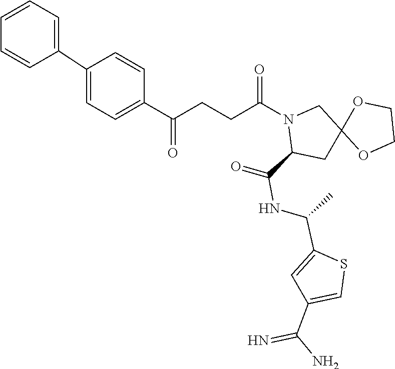 [SMILES: C[C@@H](NC(=O)[C@@H]1CC2(CN1C(=O)CCC(=O)c1ccc(-c3ccccc3)cc1)OCCO2)c1cc(C(=N)N)cs1] | (S)-7-(4-([1,1′- biphenyl]-4-yl)-4- oxobutanoyl)-N-((R)-1- (4- carbamimidoylthiophen- 2-yl)ethyl)-1,4-dioxa-7- azaspiro[4.4]nonane-8- carboxamide | *** | 1.44 | 561 |
| 346 | 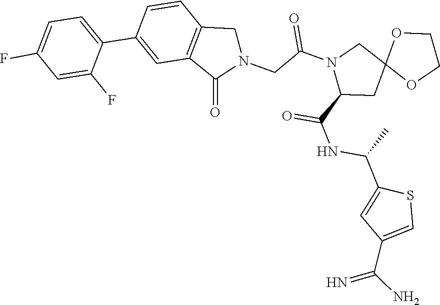 [SMILES: C[C@@H](NC(=O)[C@@H]1CC2(CN1C(=O)CN1Cc3ccc(-c4ccc(F)cc4F)cc3C1=O)OCCO2)c1cc(C(=N)N)cs1] | (S)-N-((R)-1-(4- carbamimidoylthiophen- 2-yl)ethyl)-7-(2-(6-(2,4- difluorophenyl)-1- oxoisoindolin-2- yl)acetyl)-1,4-dioxa-7- azaspiro[4.4]nonane-8- carboxamide | *** | 1.37 | 610 |
| 347 | 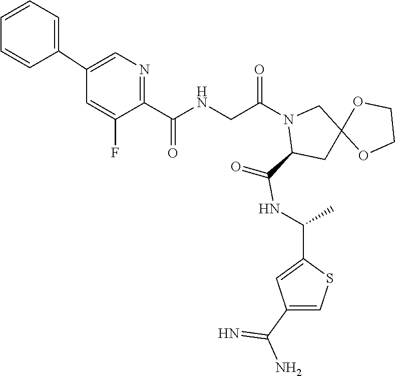 [SMILES: C[C@@H](NC(=O)[C@@H]1CC2(CN1C(=O)CNC(=O)c1ncc(-c3ccccc3)cc1F)OCCO2)c1cc(C(=N)N)cs1] | (S)-N-((R)-1-(4- carbamimidoylthiophen- 2-yl)ethyl)-7-((3-fluoro- 5- phenylpicolinoyl)glycyl)- 1,4-dioxa-7- azaspiro[4.4]nonane-8- carboxamide | *** | 1.17 | 581 |
| 348 | 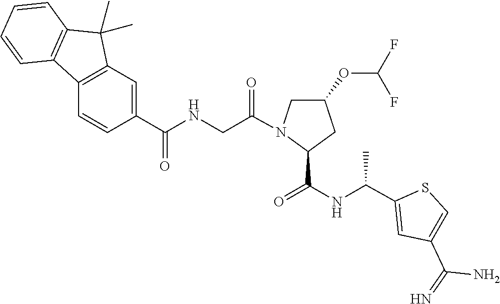 [SMILES: C[C@@H](NC(=O)[C@@H]1C[C@@H](OC(F)F)CN1C(=O)CNC(=O)c1ccc2c(c1)C(C)(C)c1ccccc1-2)c1cc(C(=N)N)cs1] | (2S,4R)-N-((R)-1-(4- carbamimidoylthiophen- 2-yl)ethyl)-4- (difluoromethoxy)-1- ((9,9-dimethyl-9H- fluorene-2- carbonyl)glycyl) pyrrolidine-2- carboxamide | *** | 1.45 | 610 |
| 349 | 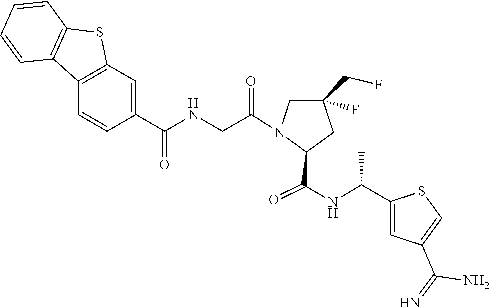 [SMILES: C[C@@H](NC(=O)[C@@H]1C[C@](F)(CF)CN1C(=O)CNC(=O)c1ccc2c(c1)sc1ccccc12)c1cc(C(=N)N)cs1] | (2S,4R)-N-((R)-1-(4- carbamimidoylthiophen- 2-yl)ethyl)-1- ((dibenzo[b,d]thiophene- 3-carbonyl)glycyl)-4- fluoro-4- (fluoromethyl) pyrrolidine-2- carboxamide | *** | 1.25 | 584 |
| 350 | 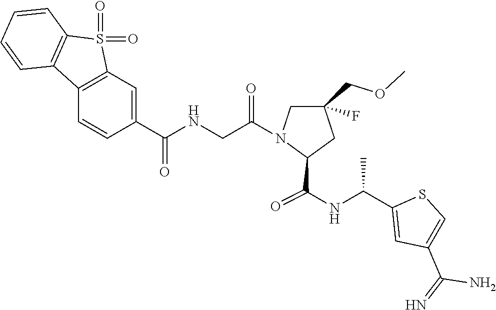 [SMILES: COC[C@@]1(F)C[C@@H](C(=O)N[C@H](C)c2cc(C(=N)N)cs2)N(C(=O)CNC(=O)c2ccc3c(c2)S(=O)(=O)c2ccccc2-3)C1] | (2S,4R)-N-((R)-1-(4- carbamimidoylthiophen- 2-yl)ethyl)-1-((5,5- dioxidodibenzo[b,d] thiophene-3- carbonyl)glycyl)-4- fluoro-4- (methoxymethyl) pyrrolidine-2- carboxamide | *** | 0.87 | 628 |
| 351 | 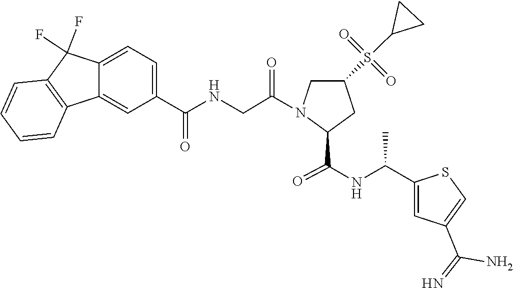 [SMILES: C[C@@H](NC(=O)[C@@H]1C[C@@H](S(=O)(=O)C2CC2)CN1C(=O)CNC(=O)c1ccc2c(c1)-c1ccccc1C2(F)F)c1cc(C(=N)N)cs1] | (2S,4R)-N-((R)-1-(4- carbamimidoylthiophen- 2-yl)ethyl)-4- (cyclopropylsulfonyl)-1- ((9,9-difluoro-9H- fluorene-3- carbonyl)glycyl) pyrrolidine-2- carboxamide | *** | 1.29 | 656 |
| 352 | 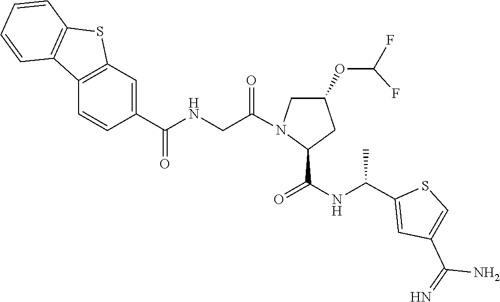 [SMILES: C[C@@H](NC(=O)[C@@H]1C[C@@H](OC(F)F)CN1C(=O)CNC(=O)c1ccc2c(c1)sc1ccccc12)c1cc(C(=N)N)cs1] | (2S,4R)-N-((R)-1-(4- carbamimidoylthiophen- 2-yl)ethyl)-1- ((dibenzo[b,d]thiophene- 3-carbonyl)glycyl)-4- (difluoromethoxy) pyrrolidine-2- carboxamide | *** | 1.28 | 600 |
| 353 | 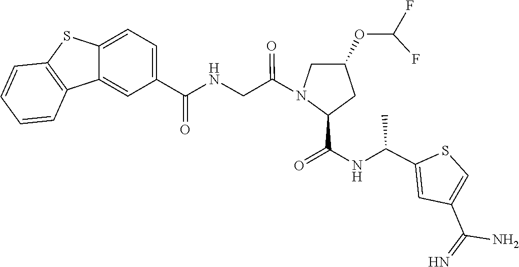 [SMILES: C[C@@H](NC(=O)[C@@H]1C[C@@H](OC(F)F)CN1C(=O)CNC(=O)c1ccc2sc3ccccc3c2c1)c1cc(C(=N)N)cs1] | (2S,4R)-N-((R)-1-(4- carbamimidoylthiophen- 2-yl)ethyl)-1- ((dibenzo[b,d]thiophene- 2-carbonyl)glycyl)-4- (difluoromethoxy) pyrrolidine-2- carboxamide | *** | 1.33 | 600 |
| 354 | 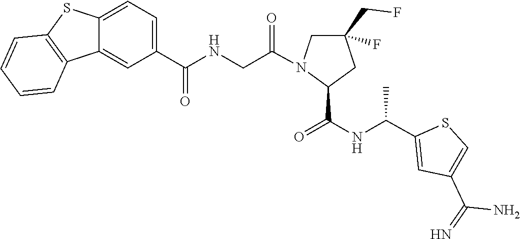 [SMILES: C[C@@H](NC(=O)[C@@H]1C[C@](F)(CF)CN1C(=O)CNC(=O)c1ccc2sc3ccccc3c2c1)c1cc(C(=N)N)cs1] | (2S,4R)-N-((R)-1-(4- carbamimidoylthiophen- 2-yl)ethyl)-1- ((dibenzo[b,d]thiophene- 2-carbonyl)glycyl)-4- fluoro-4- (fluoromethyl) pyrrolidine-2- carboxamide | *** | 1.29 | 585 |
| 355 | 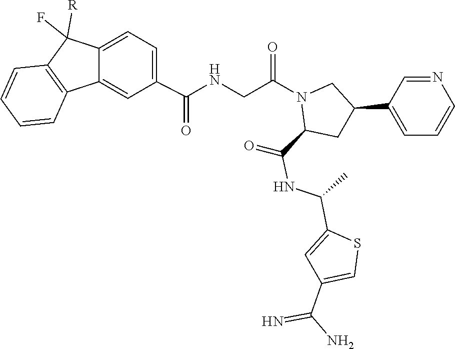 | (2S,4R)-N-((R)-1-(4- carbamimidoylthiophen- 2-yl)ethyl)-1-((9,9- difluoro-9H-fluorene-3- carbonyl)glycyl)-4- (pyridin-3-yl)pyrrolidine- 2-carboxamide | *** | 1.19 | 629 |
| 356 | 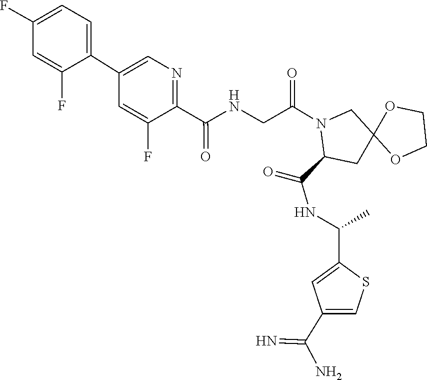 [SMILES: C[C@@H](NC(=O)[C@@H]1CC2(CN1C(=O)CNC(=O)c1ncc(-c3ccc(F)cc3F)cc1F)OCCO2)c1cc(C(=N)N)cs1] | (S)-N-((R)-1-(4- carbamimidoylthiophen- 2-yl)ethyl)-7-((5-(2,4- difluorophenyl)-3- fluoropicolinoyl)glycyl)- 1,4-dioxa-7- azaspiro[4.4]nonane-8- carboxamide | *** | | 617 |
| 357 | 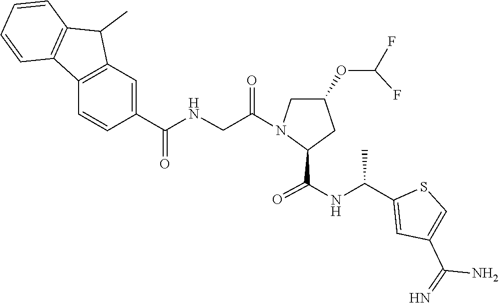 [SMILES: CC1c2ccccc2-c2ccc(C(=O)NCC(=O)N3C[C@H](OC(F)F)C[C@H]3C(=O)N[C@H](C)c3cc(C(=N)N)cs3)cc21] | (2S,4R)-N-((R)-1-(4- carbamimidoylthiophen- 2-yl)ethyl)-4- (difluoromethoxy)-1-((9- methyl-9H-fluorene-2- carbonyl)glycyl) pyrrolidine-2- carboxamide | *** | 1.41 | 596 |
| 358 | 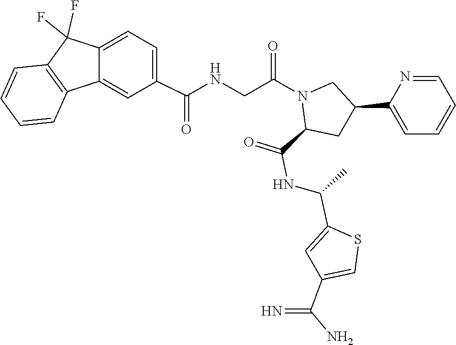 [SMILES: C[C@@H](NC(=O)[C@@H]1C[C@H](c2ccccn2)CN1C(=O)CNC(=O)c1ccc2c(c1)-c1ccccc1C2(F)F)c1cc(C(=N)N)cs1] | (2S,4S)-N-((R)-1-(4- carbamimidoylthiophen- 2-yl)ethyl)-1-((9,9- difluoro-9H-fluorene-3- carbonyl)glycyl)-4- (pyridin-2-yl)pyrrolidine- 2-carboxamide | *** | 1.28 | 629 |
| 359 | 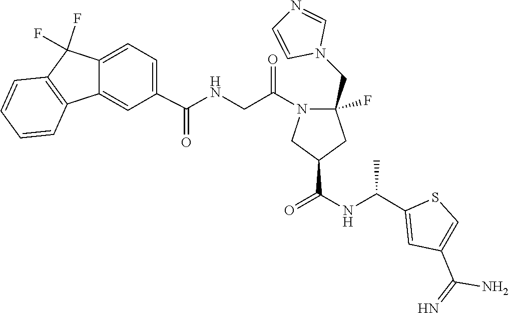 [SMILES: C[C@@H](NC(=O)[C@H]1CN(C(=O)CNC(=O)c2ccc3c(c2)-c2ccccc2C3(F)F)[C@@](F)(Cn2ccnc2)C1)c1cc(C(=N)N)cs1] | (2S,4S)-4-((1H- imidazol-1-yl)methyl)- N-((R)-1-(4- carbamimidoylthiophen- 2-yl)ethyl)-1-((9,9- difluoro-9H-fluorene-3- carbonyl)glycyl)-4- fluoropyrrolidine-2- carboxamide | *** | 0.78 | 650 |
| 360 | 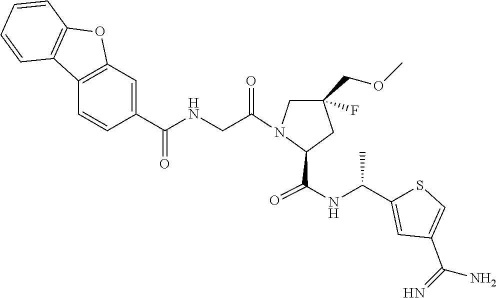 [SMILES: COC[C@@]1(F)C[C@@H](C(=O)N[C@H](C)c2cc(C(=N)N)cs2)N(C(=O)CNC(=O)c2ccc3c(c2)oc2ccccc23)C1] | (2S,4R)-N-((R)-1-(4- carbamimidoylthiophen- 2-yl)ethyl)-1- ((dibenzo[b,d]furan-3- carbonyl)glycyl)-4- fluoro-4- (methoxymethyl) pyrrolidine-2- carboxamide | *** | 1.15 | 580 |
| 361 | 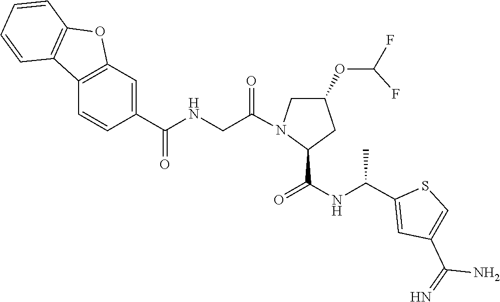 [SMILES: C[C@@H](NC(=O)[C@@H]1C[C@@H](OC(F)F)CN1C(=O)CNC(=O)c1ccc2c(c1)oc1ccccc12)c1cc(C(=N)N)cs1] | (2S,4R)-N-((R)-1-(4- carbamimidoylthiophen- 2-yl)ethyl)-1- ((dibenzo[b,d]furan-3- carbonyl)glycyl)-4- (difluoromethoxy) pyrrolidine-2- carboxamide | *** | 1.23 | 584 |
| 362 | 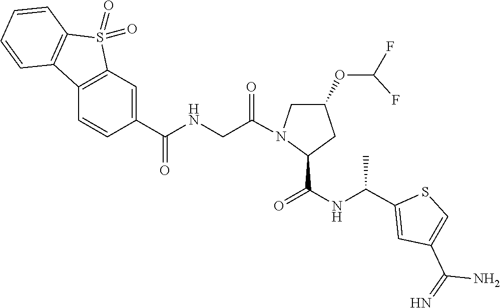 [SMILES: C[C@@H](NC(=O)[C@@H]1C[C@@H](OC(F)F)CN1C(=O)CNC(=O)c1ccc2c(c1)S(=O)(=O)c1ccccc1-2)c1cc(C(=N)N)cs1] | (2S,4R)-N-((R)-1-(4- carbamimidoylthiophen- 2-yl)ethyl)-4- (difluoromethoxy)-1- ((5,5- dioxidodibenzo[b,d] thiophene-3- carbonyl)glycyl) pyrrolidine-2- carboxamide | *** | 0.96 | 632 |
| 363 | 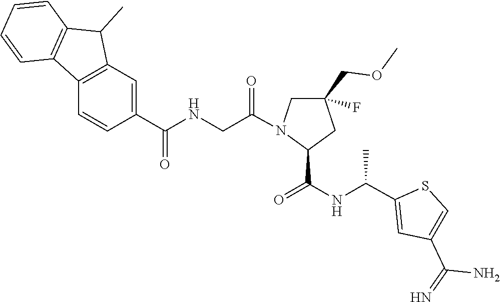 [SMILES: COC[C@@]1(F)C[C@@H](C(=O)N[C@H](C)c2cc(C(=N)N)cs2)N(C(=O)CNC(=O)c2ccc3c(c2)C(C)c2ccccc2-3)C1] | (2S,4R)-N-((R)-1-(4- carbamimidoylthiophen- 2-yl)ethyl)-4-fluoro-4- (methoxymethyl)-1-((9- methyl-9H-fluorene-2- carbonyl)glycyl) pyrrolidine-2- carboxamide | *** | 1.30 | 592 |
| 364 | 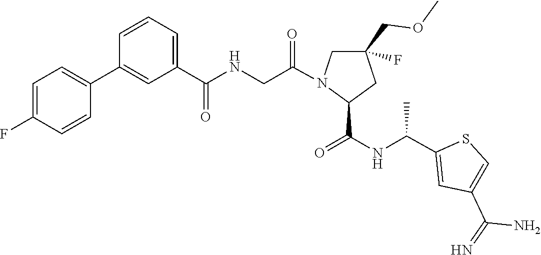 [SMILES: COC[C@@]1(F)C[C@@H](C(=O)N[C@H](C)c2cc(C(=N)N)cs2)N(C(=O)CNC(=O)c2cccc(-c3ccc(F)cc3)c2)C1] | (2S,4R)-N-((R)-1-(4- carbamimidoylthiophen- 2-yl)ethyl)-4-fluoro-1- ((4′-fluoro-[1,1′- biphenyl]-3- carbonyl)glycyl)-4- (methoxymethyl) pyrrolidine-2- carboxamide | *** | 1.19 | 584 |
| 365 | 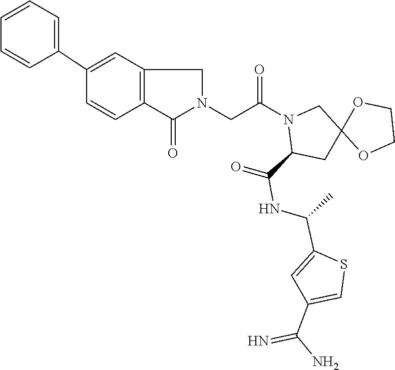 [SMILES: C[C@@H](NC(=O)[C@@H]1CC2(CN1C(=O)CN1Cc3cc(-c4ccccc4)ccc3C1=O)OCCO2)c1cc(C(=N)N)cs1] | (S)-N-((R)-1-(4- carbamimidoylthiophen- 2-yl)ethyl)-7-(2-(1-oxo- 5-phenylisoindolin-2- yl)acetyl)-1,4-dioxa-7- azaspiro[4.4]nonane-8- carboxamide | *** | 1.28 | 574 |
| 366 | 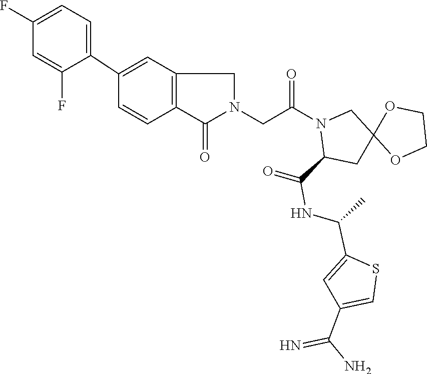 [SMILES: C[C@@H](NC(=O)[C@@H]1CC2(CN1C(=O)CN1Cc3cc(-c4ccc(F)cc4F)ccc3C1=O)OCCO2)c1cc(C(=N)N)cs1] | (S)-N-((R)-1-(4- carbamimidoylthiophen- 2-yl)ethyl)-7-(2-(5-(2,4- difluorophenyl)-1- oxoisoindolin-2- yl)acetyl)-1,4-dioxa-7- azaspiro[4.4]nonane-8- carboxamide | *** | 1.36 | 610 |
| 367 | 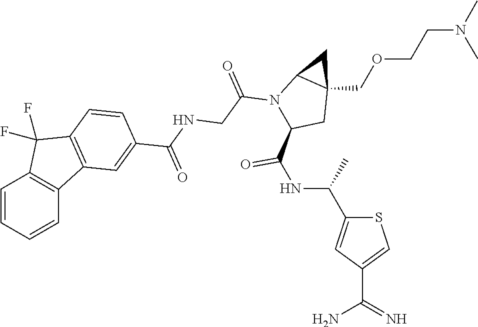 [SMILES: C[C@@H](NC(=O)[C@@H]1C[C@]2(COCCN(C)C)C[C@@H]2N1C(=O)CNC(=O)c1ccc2c(c1)-c1ccccc1C2(F)F)c1cc(C(=N)N)cs1] | (1S,3S,5R)-N-((R)-1-(4- carbamimidoylthiophen- 2-yl)ethyl)-2-((9,9- difluoro-9H-fluorene-3- carbonyl)glycyl)-5-((2- (dimethylamino)ethoxy) methyl)-2- azabicyclo[3.1.0]hexane- 3-carboxamide | *** | 0.84 | 665 |
| 368 | 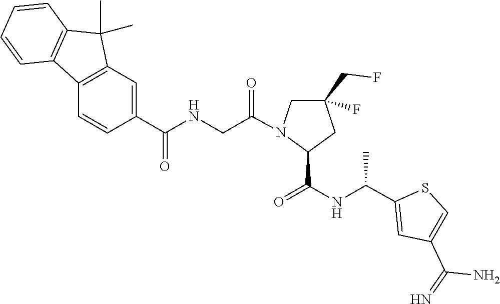 [SMILES: C[C@@H](NC(=O)[C@@H]1C[C@](F)(CF)CN1C(=O)CNC(=O)c1ccc2c(c1)C(C)(C)c1ccccc1-2)c1cc(C(=N)N)cs1] | (2S,4R)-N-((R)-1-(4- carbamimidoylthiophen- 2-yl)ethyl)-1-((9,9- dimethyl-9H-fluorene-2- carbonyl)glycyl)-4- fluoro-4- (fluoromethyl)pyrrolidine- 2-carboxamide | *** | 1.41 | 594 |
| 369 | 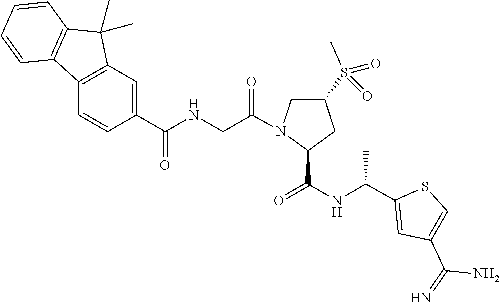 [SMILES: C[C@@H](NC(=O)[C@@H]1C[C@@H](S(C)(=O)=O)CN1C(=O)CNC(=O)c1ccc2c(c1)C(C)(C)c1ccccc1-2)c1cc(C(=N)N)cs1] | (2S,4R)-N-((R)-1-(4- carbamimidoylthiophen- 2-yl)ethyl)-1-((9,9- dimethyl-9H-fluorene-2- carbonyl)glycyl)-4- (methylsulfonyl) pyrrolidine-2- carboxamide | *** | 1.27 | 622 |
| 370 | 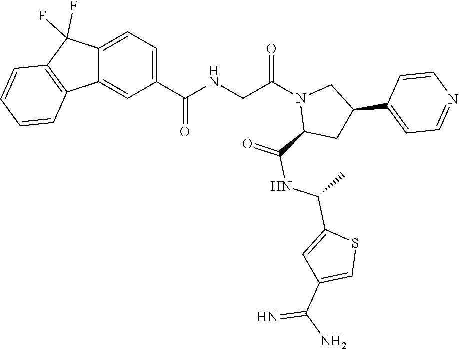 [SMILES: C[C@@H](NC(=O)[C@@H]1C[C@H](c2ccncc2)CN1C(=O)CNC(=O)c1ccc2c(c1)-c1ccccc1C2(F)F)c1cc(C(=N)N)cs1] | (2S,4R)-N-((R)-1-(4- carbamimidoylthiophen- 2-yl)ethyl)-1-((9,9- difluoro-9H-fluorene-3- carbonyl)glycyl)-4- (pyridin-4-yl)pyrrolidine- 2-carboxamide | *** | 1.03 | 629 |
| 371 | 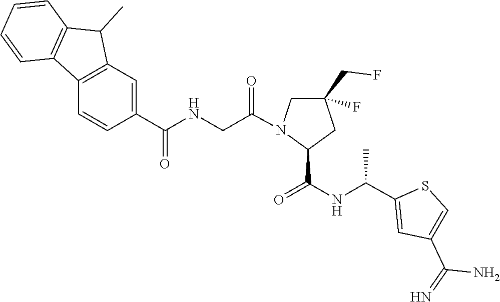 [SMILES: CC1c2ccccc2-c2ccc(C(=O)NCC(=O)N3C[C@@](F)(CF)C[C@H]3C(=O)N[C@H](C)c3cc(C(=N)N)cs3)cc21] | (2S,4R)-N-((R)-1-(4- carbamimidoylthiophen- 2-yl)ethyl)-4-fluoro-4- (fluoromethyl)-1-((9- methyl-9H-fluorene-2- carbonyl)glycyl) pyrrolidine-2- carboxamide | *** | 1.32 | 580 |
| 372 | 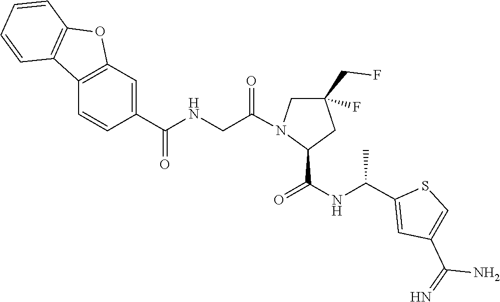 [SMILES: C[C@@H](NC(=O)[C@@H]1C[C@](F)(CF)CN1C(=O)CNC(=O)c1ccc2c(c1)oc1ccccc12)c1cc(C(=N)N)cs1] | (2S,4R)-N-((R)-1-(4- carbamimidoylthiophen- 2-yl)ethyl)-1- ((dibenzo[b,d]furan-3- carbonyl)glycyl)-4- fluoro-4- (fluoromethyl)pyrrolidine- 2-carboxamide | *** | 1.16 | 568 |
| 373 | 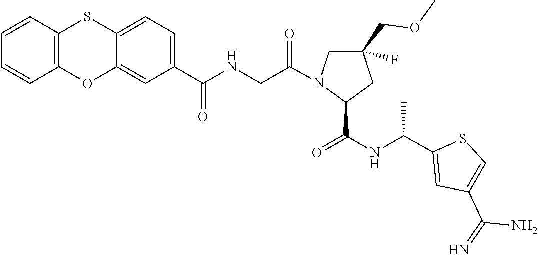 [SMILES: COC[C@@]1(F)C[C@@H](C(=O)N[C@H](C)c2cc(C(=N)N)cs2)N(C(=O)CNC(=O)c2ccc3c(c2)Oc2ccccc2S3)C1] | (2S,4R)-N-((R)-1-(4- carbamimidoylthiophen- 2-yl)ethyl)-4-fluoro-4- (methoxymethyl)-1- ((phenoxathiine-3- carbonyl)glycyl) pyrrolidine-2- carboxamide | *** | 1.31 | 612 |
| 374 | 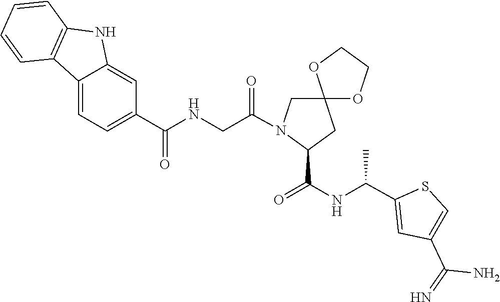 [SMILES: C[C@@H](NC(=O)[C@@H]1CC2(CN1C(=O)CNC(=O)c1ccc3c(c1)[nH]c1ccccc13)OCCO2)c1cc(C(=N)N)cs1] | (S)-7-((9H-carbazole-2- carbonyl)glycyl)-N-((R)- 1-(4- carbamimidoylthiophen- 2-yl)ethyl)-1,4-dioxa-7- azaspiro[4.4]nonane-8- carboxamide | *** | 1.03 | 575 |
| 375 | 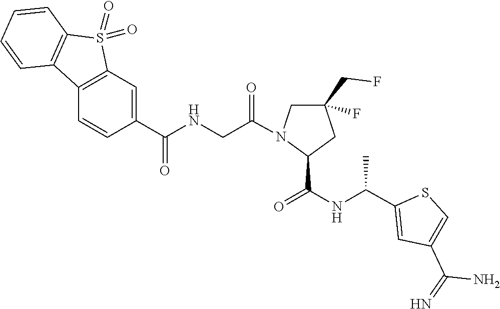 [SMILES: C[C@@H](NC(=O)[C@@H]1C[C@](F)(CF)CN1C(=O)CNC(=O)c1ccc2c(c1)S(=O)(=O)c1ccccc1-2)c1cc(C(=N)N)cs1] | (2S,4R)-N-((R)-1-(4- carbamimidoylthiophen- 2-yl)ethyl)-1-((5,5- dioxidodibenzo[b,d] thiophene-3- carbonyl)glycyl)-4- fluoro-4- (fluoromethyl)pyrrolidine- 2-carboxamide | *** | 0.89 | 616 |
| 376 | 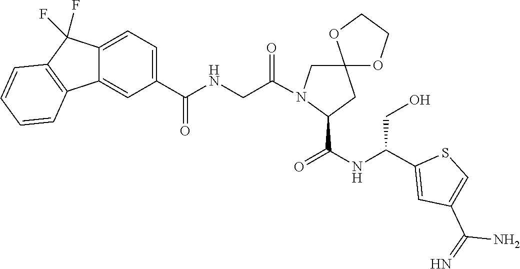 [SMILES: N=C(N)c1csc([C@@H](CO)NC(=O)[C@@H]2CC3(CN2C(=O)CNC(=O)c2ccc4c(c2)-c2ccccc2C4(F)F)OCCO3)c1] | (S)-N-((R)-1-(4- carbamimidoylthiophen- 2-yl)-2-hydroxyethyl)- 7-((9,9-difluoro-9H- fluorene-3- carbonyl)glycyl)-1,4- dioxa-7- azaspiro[4.4]nonane-8- carboxamide | *** | 1.32 | 626 |
| 377 | 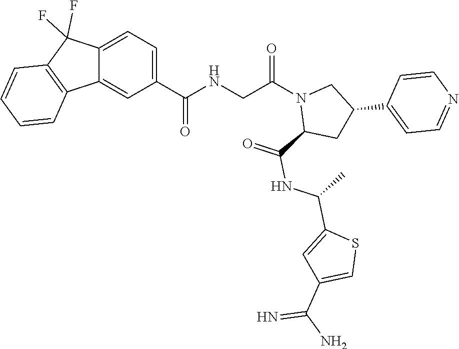 [SMILES: C[C@@H](NC(=O)[C@@H]1C[C@@H](c2ccncc2)CN1C(=O)CNC(=O)c1ccc2c(c1)-c1ccccc1C2(F)F)c1cc(C(=N)N)cs1] | (2S,4S)-N-((R)-1-(4- carbamimidoylthiophen- 2-yl)ethyl)-1-((9,9- difluoro-9H-fluorene-3- carbonyl)glycyl)-4- (pyridin-4-yl)pyrrolidine- 2-carboxamide | *** | 1.18 | 629 |
| 378 | 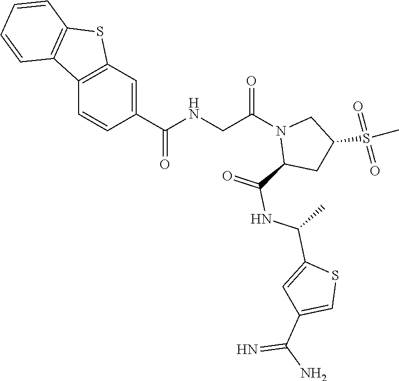 [SMILES: C[C@@H](NC(=O)[C@@H]1C[C@@H](S(C)(=O)=O)CN1C(=O)CNC(=O)c1ccc2c(c1)sc1ccccc12)c1cc(C(=N)N)cs1] | (2S,4R)-N-((R)-1-(4- carbamimidoylthiophen- 2-yl)ethyl)-1- ((dibenzo[b,d]thiophene- 3-carbonyl)glycyl)-4- (methylsulfonyl) pyrrolidine-2- carboxamide | *** | 1.29 | 612 |
| 379 | 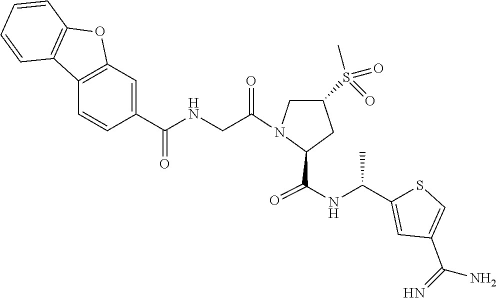 [SMILES: C[C@@H](NC(=O)[C@@H]1C[C@@H](S(C)(=O)=O)CN1C(=O)CNC(=O)c1ccc2c(c1)oc1ccccc12)c1cc(C(=N)N)cs1] | (2S,4R)-N-((R)-1-(4- carbamimidoylthiophen- 2-yl)ethyl)-1- ((dibenzo[b,d]furan-3- carbonyl)glycyl)-4- (methylsulfonyl) pyrrolidine-2- carboxamide | *** | 1.20 | 596 |
| 380 | 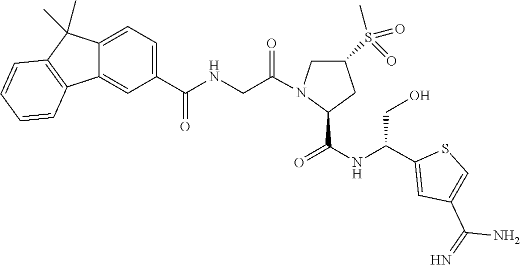 [SMILES: CC1(C)c2ccccc2-c2cc(C(=O)NCC(=O)N3C[C@H](S(C)(=O)=O)C[C@H]3C(=O)N[C@H](CO)c3cc(C(=N)N)cs3)ccc21] | (2S,4R)-N-((R)-1-(4- carbamimidoylthiophen- 2-yl)-2-hydroxyethyl)- 1-((9,9-dimethyl-9H- fluorene-3- carbonyl)glycyl)-4- (methylsulfonyl) pyrrolidine-2- carboxamide | *** | 1.36 | 638 |
| 381 | 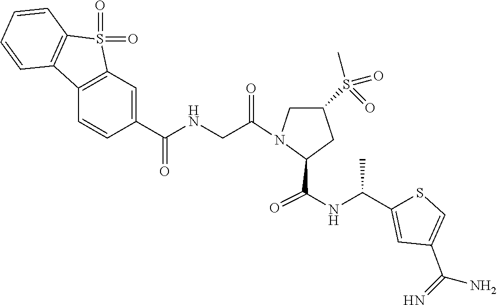 [SMILES: C[C@@H](NC(=O)[C@@H]1C[C@@H](S(C)(=O)=O)CN1C(=O)CNC(=O)c1ccc2c(c1)S(=O)(=O)c1ccccc1-2)c1cc(C(=N)N)cs1] | (2S,4R)-N-((R)-1-(4- carbamimidoylthiophen- 2-yl)ethyl)-1-((5,5- dioxidodibenzo[b,d] thiophene-3- carbonyl)glycyl)-4- (methylsulfonyl) pyrrolidine-2- carboxamide | *** | 0.90 | 644 |
| 382 | 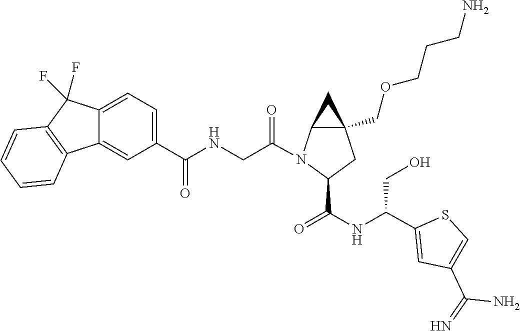 [SMILES: N=C(N)c1csc([C@@H](CO)NC(=O)[C@@H]2C[C@]3(COCCCN)C[C@@H]3N2C(=O)CNC(=O)c2ccc3c(c2)-c2ccccc2C3(F)F)c1] | (1S,3S,5R)-5-((3- aminopropoxy)methyl)- N-((R)-1-(4- carbamimidoylthiophen- 2-yl)ethyl)-2-((9,9- difluoro-9H-fluorene-3- carbonyl)glycyl)-2- azabicyclo[3.1.0]hexane- 3-carboxamide | *** | 1.04 | 651 |
| 383 | 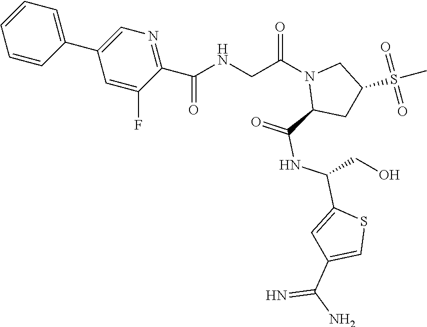 [SMILES: CS(=O)(=O)[C@@H]1C[C@@H](C(=O)N[C@H](CO)c2cc(C(=N)N)cs2)N(C(=O)CNC(=O)c2ncc(-c3ccccc3)cc2F)C1] | N-(2-((2S,4R)-2-(((R)-1- (4- carbamimidoylthiophen- 2-yl)-2- hydroxyethyl)carbamoyl)- 4- (methylsulfonyl) pyrrolidin-1-yl)- 2-oxoethyl)-3- fluoro-5- phenylpicolinamide | *** | 0.98 | 617 |
| 384 | 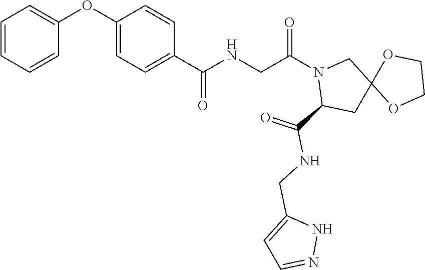 [SMILES: O=C(NCC(=O)N1CC2(C[C@H]1C(=O)NCc1ccn[nH]1)OCCO2)c1ccc(Oc2ccccc2)cc1] | (S)-N-((1H-pyrazol-5- yl)methyl)-7-((4- phenoxybenzoyl)glycyl)- 1,4-dioxa-7- azaspiro[4.4]nonane-8- carboxamide | * | 1.52 | 506 |
| 385 | 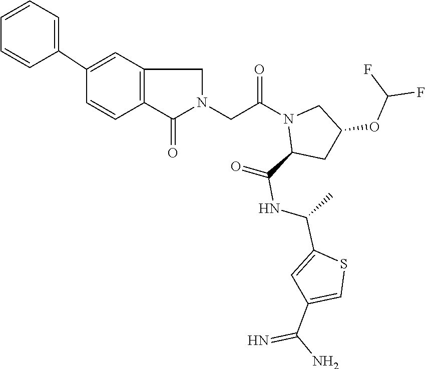 [SMILES: C[C@@H](NC(=O)[C@@H]1C[C@@H](OC(F)F)CN1C(=O)CN1Cc2cc(-c3ccccc3)ccc2C1=O)c1cc(C(=N)N)cs1] | (2S,4R)-N-((R)-1-(4- carbamimidoylthiophen- 2-yl)ethyl)-4- (difluoromethoxy)-1-(2- (1-oxo-5- phenylisoindolin-2- yl)acetyl)pyrrolidine-2- carboxamide | *** | 1.41 | 582 |
| 386 | 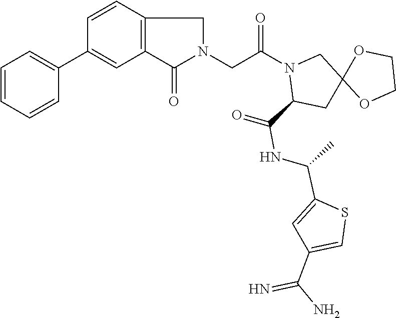 [SMILES: C[C@@H](NC(=O)[C@@H]1CC2(CN1C(=O)CN1Cc3ccc(-c4ccccc4)cc3C1=O)OCCO2)c1cc(C(=N)N)cs1] | (S)-N-((R)-1-(4- carbamimidoylthiophen- 2-yl)ethyl)-7-(2-(1-oxo- 6-phenylisoindolin-2- yl)acetyl)-1,4-dioxa-7- azaspiro[4.4]nonane-8- carboxamide | *** | 1.24 | 574 |
| 387 | 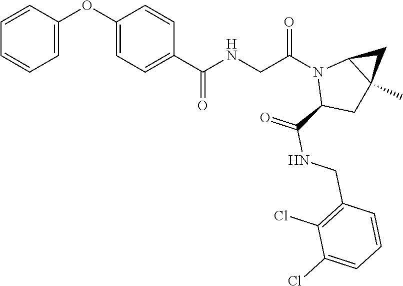 [SMILES: C[C@@]12C[C@@H]1N(C(=O)CNC(=O)c1ccc(Oc3ccccc3)cc1)[C@H](C(=O)NCc1cccc(Cl)c1Cl)C2] | (1S,3S,5S)-N-(2,3- dichlorobenzyl)-5- methyl-2-((4- phenoxybenzoyl)glycyl)- 2- azabicyclo[3.1.0]hexane- 3-carboxamide | * | 2.63 | 552 |
| 388 | 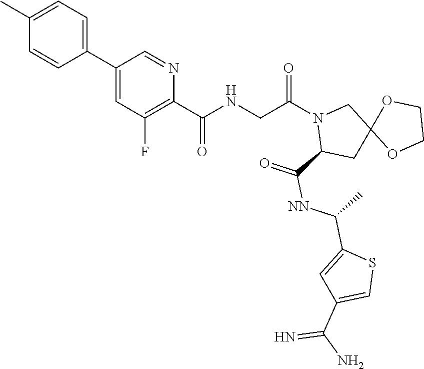 | (S)-N-((R)-1-(4- carbamimidoylthiophen- 2-yl)ethyl)-7-((3-fluoro- 5-(p- tolyl)picolinoyl)glycyl)- 1,4-dioxa-7- azaspiro[4.4]nonane-8- carboxamide | *** | 1.33 | 595 |
| 389 | 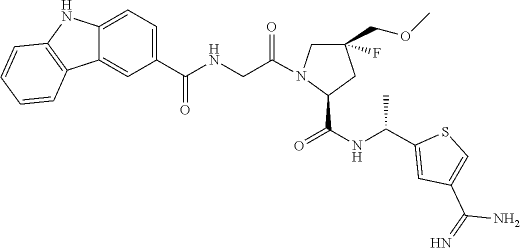 [SMILES: COC[C@@]1(F)C[C@@H](C(=O)N[C@H](C)c2cc(C(=N)N)cs2)N(C(=O)CNC(=O)c2ccc3[nH]c4ccccc4c3c2)C1] | N-(2-((2S,4R)-2-(((R)-1- (4- carbamimidoylthiophen- 2-yl)ethyl)carbamoyl)- 4-fluoro-4- (methoxymethyl) pyrrolidin-1-yl)- 2-oxoethyl)- 9H-carbazole-3- carboxamide | *** | 1.14 | 579 |
| 390 | 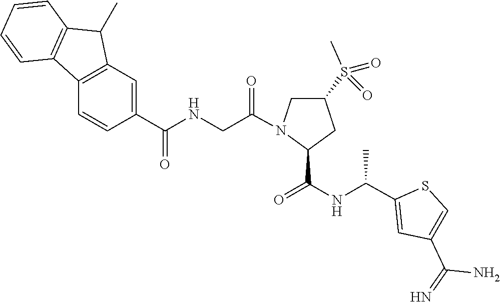 [SMILES: CC1c2ccccc2-c2ccc(C(=O)NCC(=O)N3C[C@H](S(C)(=O)=O)C[C@H]3C(=O)N[C@H](C)c3cc(C(=N)N)cs3)cc21] | (2S,4R)-N-((R)-1-(4- carbamimidoylthiophen- 2-yl)ethyl)-1-((9- methyl-9H-fluorene-2- carbonyl)glycyl)-4- (methylsulfonyl) pyrrolidine-2- carboxamide | *** | 1.36 | 608 |
| 391 | 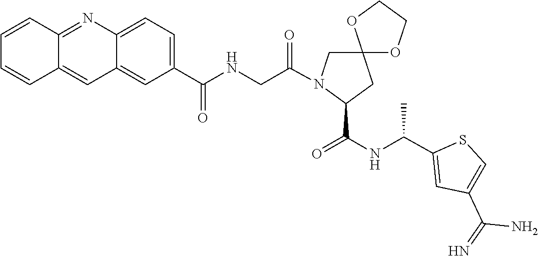 [SMILES: C[C@@H](NC(=O)[C@@H]1CC2(CN1C(=O)CNC(=O)c1ccc3nc4ccccc4cc3c1)OCCO2)c1cc(C(=N)N)cs1] | (S)-7-((acridine-2- carbonyl)glycyl)-N-((R)- 1-(4- carbamimidoylthiophen- 2-yl)ethyl)-1,4-dioxa-7- azaspiro[4.4]nonane-8- carboxamide | *** | 0.86 | 587 |
| 392 | 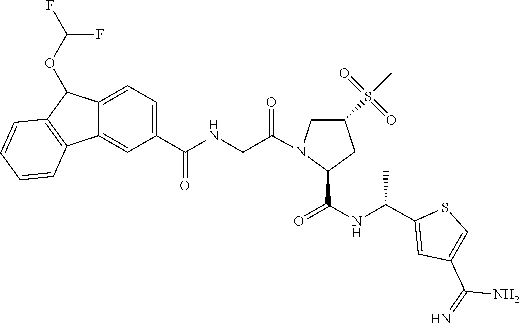 [SMILES: C[C@@H](NC(=O)[C@@H]1C[C@@H](S(C)(=O)=O)CN1C(=O)CNC(=O)c1ccc2c(c1)-c1ccccc1C2OC(F)F)c1cc(C(=N)N)cs1] | (2S,4R)-N-((R)-1-(4- carbamimidoylthiophen- 2-yl)ethyl)-1-((9- (difluoromethoxy)-9H- fluorene-3- carbonyl)glycyl)-4- (methylsulfonyl) pyrrolidine-2- carboxamide | *** | 1.33 | 660 |
| 393 | 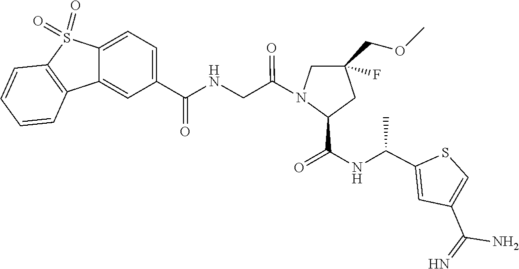 [SMILES: COC[C@@]1(F)C[C@@H](C(=O)N[C@H](C)c2cc(C(=N)N)cs2)N(C(=O)CNC(=O)c2ccc3c(c2)-c2ccccc2S3(=O)=O)C1] | (2S,4R)-N-((R)-1-(4- carbamimidoylthiophen- 2-yl)ethyl)-1-((5,5- dioxidodibenzo[b,d] thiophene-2- carbonyl)glycyl)-4- fluoro-4- (methoxymethyl) pyrrolidine-2- carboxamide | *** | 1.07 | 628 |
| 394 | 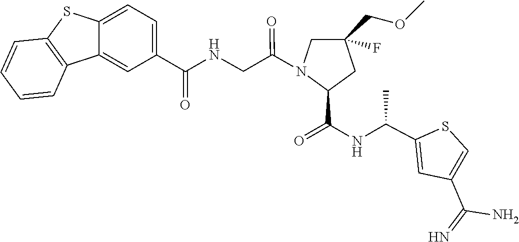 [SMILES: COC[C@@]1(F)C[C@@H](C(=O)N[C@H](C)c2cc(C(=N)N)cs2)N(C(=O)CNC(=O)c2ccc3sc4ccccc4c3c2)C1] | (2S,4R)-N-((R)-1-(4- carbamimidoylthiophen- 2-yl)ethyl)-1- ((dibenzo[b,d]thiophene- 2-carbonyl)glycyl)-4- fluoro-4- (methoxymethyl) pyrrolidine-2- carboxamide | *** | 1.42 | 596 |
| 395 | 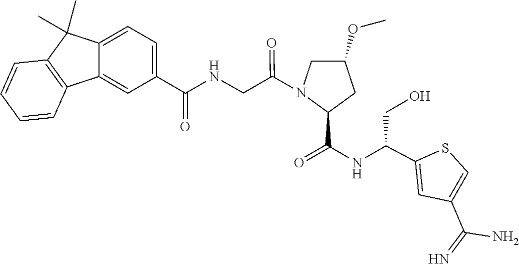 [SMILES: CO[C@@H]1C[C@@H](C(=O)N[C@H](CO)c2cc(C(=N)N)cs2)N(C(=O)CNC(=O)c2ccc3c(c2)-c2ccccc2C3(C)C)C1] | (2S,4R)-N-((R)-1-(4- carbamimidoylthiophen- 2-yl)-2-hydroxyethyl)- 1-((9,9-dimethyl-9H- fluorene-3- carbonyl)glycyl)-4- methoxypyrrolidine-2- carboxamide | *** | 1.44 | 590 |
| 396 | 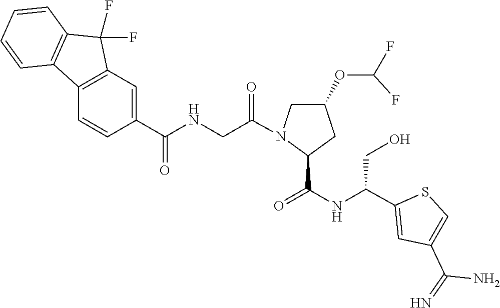 [SMILES: N=C(N)c1csc([C@@H](CO)NC(=O)[C@@H]2C[C@@H](OC(F)F)CN2C(=O)CNC(=O)c2ccc3c(c2)C(F)(F)c2ccccc2-3)c1] | (2S,4R)-N-((R)-1-(4- carbamimidoylthiophen- 2-yl)-2-hydroxyethyl)- 1-((9,9-difluoro-9H- fluorene-2- carbonyl)glycyl)-4- (difluoromethoxy) pyrrolidine-2- carboxamide | *** | 1.41 | 634 |
| 397 | 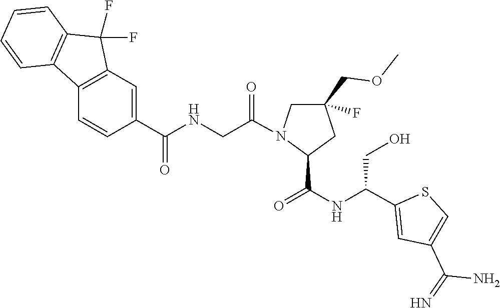 [SMILES: COC[C@@]1(F)C[C@@H](C(=O)N[C@H](CO)c2cc(C(=N)N)cs2)N(C(=O)CNC(=O)c2ccc3c(c2)C(F)(F)c2ccccc2-3)C1] | (2S,4R)-N-((R)-1-(4- carbamimidoylthiophen- 2-yl)-2-hydroxyethyl)- 1-((9,9-difluoro-9H- fluorene-2- carbonyl)glycyl)-4- fluoro-4- (methoxymethyl) pyrrolidine-2- carboxamide | *** | 1.33 | 630 |
| 398 | 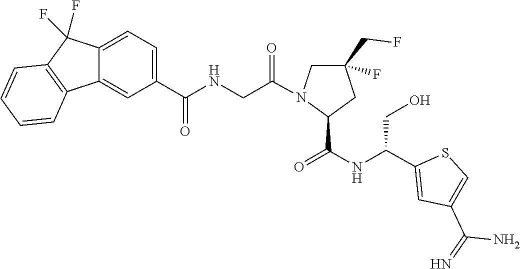 [SMILES: N=C(N)c1csc([C@@H](CO)NC(=O)[C@@H]2C[C@](F)(CF)CN2C(=O)CNC(=O)c2ccc3c(c2)-c2ccccc2C3(F)F)c1] | (2S,4R)-N-((R)-1-(4- carbamimidoylthiophen- 2-yl)-2-hydroxyethyl)- 1-((9,9-difluoro-9H- fluorene-3- carbonyl)glycyl)-4- fluoro-4- (fluoromethyl) pyrrolidine-2- carboxamide | *** | 1.44 | 618 |
| 399 | 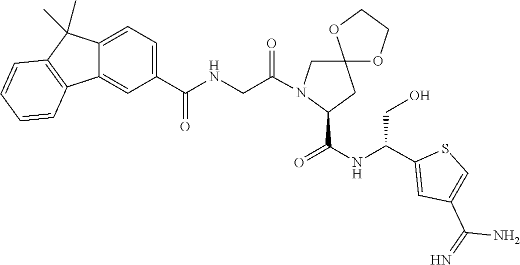 [SMILES: CC1(C)c2ccccc2-c2cc(C(=O)NCC(=O)N3CC4(C[C@H]3C(=O)N[C@H](CO)c3cc(C(=N)N)cs3)OCCO4)ccc21] | (S)-N-((R)-1-(4- carbamimidoylthiophen- 2-yl)-2-hydroxyethyl)- 7-((9,9-dimethyl-9H- fluorene-3- carbonyl)glycyl)-1,4- dioxa-7- azaspiro[4.4]nonane-8- carboxamide | *** | 1.45 | 618 |
| 400 | 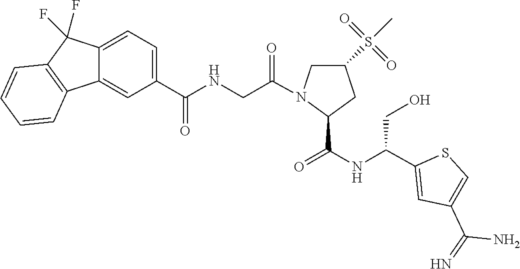 [SMILES: CS(=O)(=O)[C@@H]1C[C@@H](C(=O)N[C@H](CO)c2cc(C(=N)N)cs2)N(C(=O)CNC(=O)c2ccc3c(c2)-c2ccccc2C3(F)F)C1] | (2S,4R)-N-((R)-1-(4- carbamimidoylthiophen- 2-yl)-2-hydroxyethyl)- 1-((9,9-difluoro-9H- fluorene-3- carbonyl)glycyl)-4- (methylsulfonyl) pyrrolidine-2- carboxamide | *** | 1.26 | 646 |
| 401 | 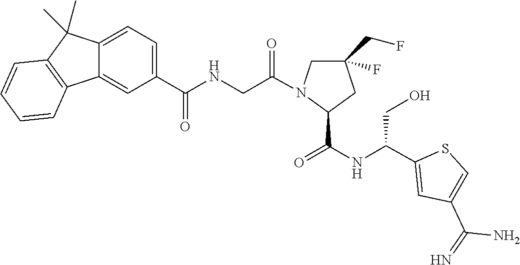 [SMILES: CC1(C)c2ccccc2-c2cc(C(=O)NCC(=O)N3C[C@@](F)(CF)C[C@H]3C(=O)N[C@H](CO)c3cc(C(=N)N)cs3)ccc21] | (2S,4R)-N-((R)-1-(4- carbamimidoylthiophen- 2-yl)-2-hydroxyethyl)- 1-((9,9-dimethyl-9H- fluorene-3- carbonyl)glycyl)-4- fluoro-4- (fluoromethyl) pyrrolidine-2- carboxamide | *** | 1.51 | 610 |
| 402 | 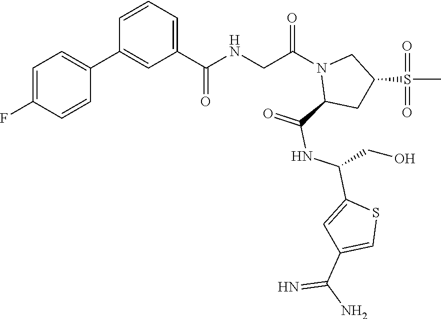 [SMILES: CS(=O)(=O)[C@@H]1C[C@@H](C(=O)N[C@H](CO)c2cc(C(=N)N)cs2)N(C(=O)CNC(=O)c2cccc(-c3ccc(F)cc3)c2)C1] | (2S,4R)-N-((R)-1-(4- carbamimidoylthiophen- 2-yl)-2-hydroxyethyl)- 1-((4′-fluoro-[1,1′- biphenyl]-3- carbonyl)glycyl)-4- (methylsulfonyl) pyrrolidine-2- carboxamide | *** | 1.15 | 616 |
| 403 | 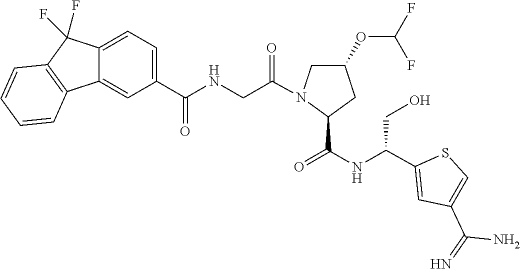 [SMILES: N=C(N)c1csc([C@@H](CO)NC(=O)[C@@H]2C[C@@H](OC(F)F)CN2C(=O)CNC(=O)c2ccc3c(c2)-c2ccccc2C3(F)F)c1] | (2S,4R)-N-((R)-1-(4- carbamimidoylthiophen- 2-yl)-2-hydroxyethyl)- 1-((9,9-difluoro-9H- fluorene-3- carbonyl)glycyl)-4- (difluoromethoxy) pyrrolidine-2- carboxamide | *** | 1.42 | 634 |
| 404 | 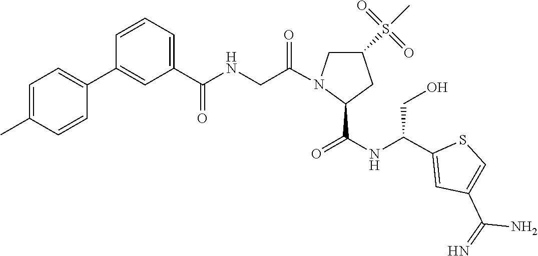 [SMILES: Cc1ccc(-c2cccc(C(=O)NCC(=O)N3C[C@H](S(C)(=O)=O)C[C@H]3C(=O)N[C@H](CO)c3cc(C(=N)N)cs3)c2)cc1] | (2S,4R)-N-((R)-1-(4- carbamimidoylthiophen- 2-yl)-2-hydroxyethyl)- 1-((4′-methyl-[1,1′- biphenyl]-3- carbonyl)glycyl)-4- (methylsulfonyl) pyrrolidine-2- carboxamide | *** | 1.25 | 612 |
| 405 | 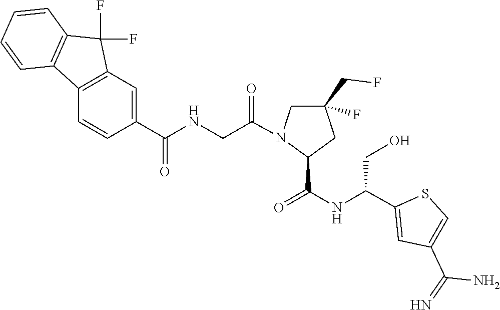 [SMILES: N=C(N)c1csc([C@@H](CO)NC(=O)[C@@H]2C[C@](F)(CF)CN2C(=O)CNC(=O)c2ccc3c(c2)C(F)(F)c2ccccc2-3)c1] | (2S,4R)-N-((R)-1-(4- carbamimidoylthiophen- 2-yl)-2-hydroxyethyl)- 1-((9,9-difluoro-9H- fluorene-2- carbonyl)glycyl)-4- fluoro-4- (fluoromethyl) pyrrolidine-2- carboxamide | *** | 1.36 | 618 |
| 406 | 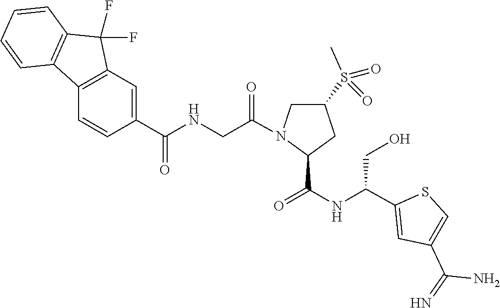 [SMILES: CS(=O)(=O)[C@@H]1C[C@@H](C(=O)N[C@H](CO)c2cc(C(=N)N)cs2)N(C(=O)CNC(=O)c2ccc3c(c2)C(F)(F)c2ccccc2-3)C1] | (2S,4R)-N-((R)-1-(4- carbamimidoylthiophen- 2-yl)-2-hydroxyethyl)- 1-((9,9-difluoro-9H- fluorene-2- carbonyl)glycyl)-4- (methylsulfonyl) pyrrolidine-2- carboxamide | *** | 1.22 | 646 |
| 407 | 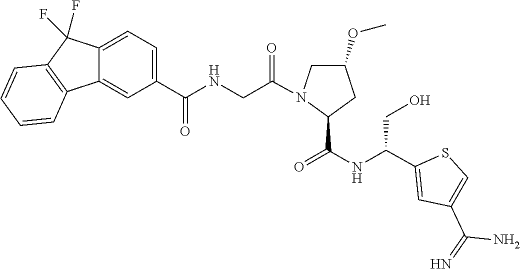 [SMILES: CO[C@@H]1C[C@@H](C(=O)N[C@H](CO)c2cc(C(=N)N)cs2)N(C(=O)CNC(=O)c2ccc3c(c2)-c2ccccc2C3(F)F)C1] | (2S,4R)-N-((R)-1-(4- carbamimidoylthiophen- 2-yl)-2-hydroxyethyl)- 1-((9,9-difluoro-9H- fluorene-3- carbonyl)glycyl)-4- methoxypyrrolidine-2- carboxamide | *** | 1.26 | 598 |
| 408 | 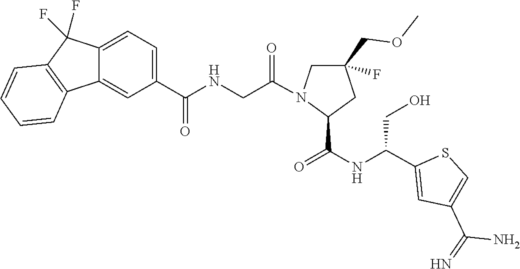 [SMILES: COC[C@@]1(F)C[C@@H](C(=O)N[C@H](CO)c2cc(C(=N)N)cs2)N(C(=O)CNC(=O)c2ccc3c(c2)-c2ccccc2C3(F)F)C1] | (2S,4R)-N-((R)-1-(4- carbamimidoylthiophen- 2-yl)-2-hydroxyethyl)- 1-((9,9-difluoro-9H- fluorene-3- carbonyl)glycyl)-4- fluoro-4- (methoxymethyl) pyrrolidine-2- carboxamide | *** | 1.34 | 630 |
| 409 | 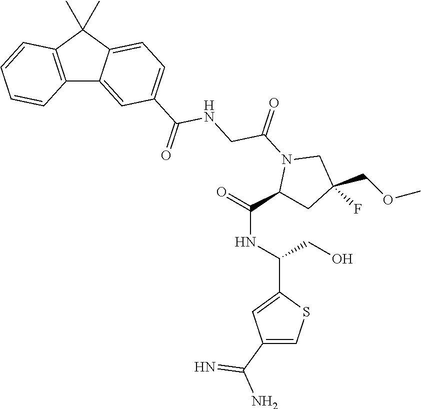 [SMILES: COC[C@@]1(F)C[C@@H](C(=O)N[C@H](CO)c2cc(C(=N)N)cs2)N(C(=O)CNC(=O)c2ccc3c(c2)-c2ccccc2C3(C)C)C1] | (2S,4R)-N-((R)-1-(4- carbamimidoylthiophen- 2-yl)-2-hydroxyethyl)- 1-((9,9-dimethyl-9H- fluorene-3- carbonyl)glycyl)-4- fluoro-4- (methoxymethyl) pyrrolidine-2- carboxamide | *** | 1.45 | 622 |
| 410 | 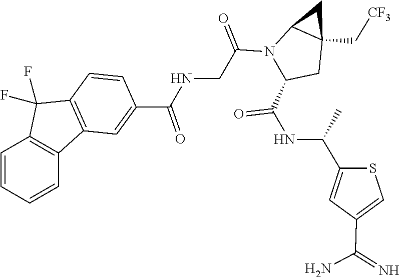 [SMILES: C[C@@H](NC(=O)[C@H]1C[C@]2(CC(F)(F)F)C[C@@H]2N1C(=O)CNC(=O)c1ccc2c(c1)-c1ccccc1C2(F)F)c1cc(C(=N)N)cs1] | (1S,3R,5S)-N-((R)-1-(4- carbamimidoylthiophen- 2-yl)ethyl)-2-((9,9- difluoro-9H-fluorene-3- carbonyl)glycyl)-5- (2,2,2-trifluoroethyl)-2- azabicyclo[3.1.0]hexane- 3-carboxamide | * | 1.65 | 646 |
| 411 | 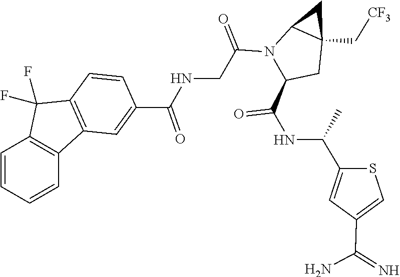 [SMILES: C[C@@H](NC(=O)[C@@H]1C[C@]2(CC(F)(F)F)C[C@@H]2N1C(=O)CNC(=O)c1ccc2c(c1)-c1ccccc1C2(F)F)c1cc(C(=N)N)cs1] | (1S,3S,5S)-N-((R)-1-(4- carbamimidoylthiophen- 2-yl)ethyl)-2-((9,9- difluoro-9H-fluorene-3- carbonyl)glycyl)-5- (2,2,2-trifluoroethyl)-2- azabicyclo[3.1.0]hexane- 3-carboxamide | * | 1.53 | 646 |
| 412 | 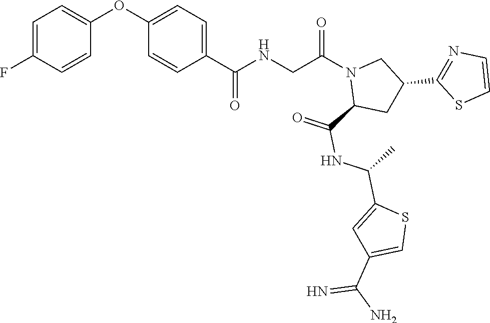 [SMILES: C[C@@H](NC(=O)[C@@H]1C[C@@H](c2nccs2)CN1C(=O)CNC(=O)c1ccc(Oc2ccc(F)cc2)cc1)c1cc(C(=N)N)cs1] | (2S,4R)-N-(R)-1-(4- carbamimidoylthiophen- 2-yl)ethyl)-1-((4-(4- fluorophenoxy)benzoyl) glycyl)-4-(thiazol-2- yl)pyrrolidine-2- carboxamide | *** | 1.40 | 621 |
| 413 | 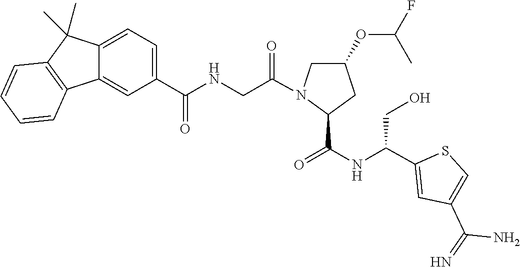 [SMILES: CC(F)O[C@@H]1C[C@@H](C(=O)N[C@H](CO)c2cc(C(=N)N)cs2)N(C(=O)CNC(=O)c2ccc3c(c2)-c2ccccc2C3(C)C)C1] | (2S,4R)-N-((R)-1-(4- carbamimidoylthiophen- 2-yl)-2-hydroxyethyl)- 4-(difluoromethoxy)-1- ((9,9-dimethyl-9H- fluorene-3- carbonyl)glycyl) pyrrolidine-2- carboxamide | *** | 1.50 | 626 |
| 414 | 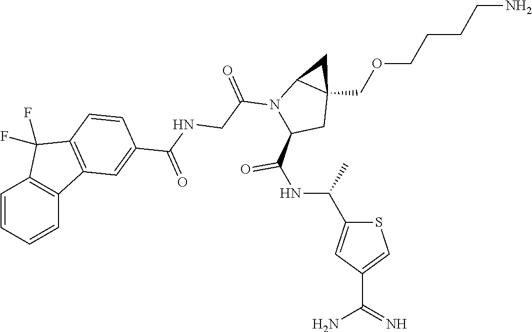 [SMILES: C[C@@H](NC(=O)[C@@H]1C[C@]2(COCCCCN)C[C@@H]2N1C(=O)CNC(=O)c1ccc2c(c1)-c1ccccc1C2(F)F)c1cc(C(=N)N)cs1] | (1S,3S,5R)-5-((4- aminobutoxy)methyl)- N-((R)-1-(4- carbamimidoylthiophen- 2-yl)ethyl)-2-((9,9- difluoro-9H-fluorene-3- carbonyl)glycyl)-2- azabicyclo[3.1.0]hexane- 3-carboxamide | *** | 1.00 | 665 |
| 415 | 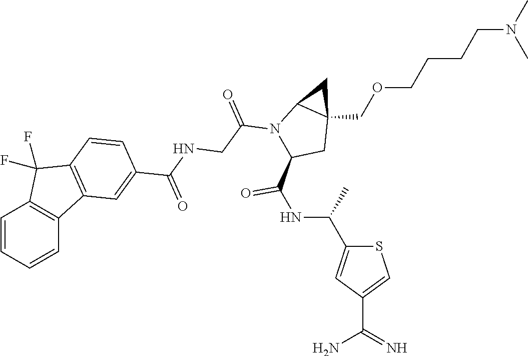 [SMILES: C[C@@H](NC(=O)[C@@H]1C[C@]2(COCCCCN(C)C)C[C@@H]2N1C(=O)CNC(=O)c1ccc2c(c1)-c1ccccc1C2(F)F)c1cc(C(=N)N)cs1] | (1S,3S,5R)-N-((R)-1-(4- carbamimidoylthiophen- 2-yl)ethyl)-2-((9,9- difluoro-9H-fluorene-3- carbonyl)glycyl)-5-((4- (dimethylamino)butoxy) methyl)-2- azabicyclo[3.1.0]hexane- 3-carboxamide | *** | 1.08 | 693 |
| 416 | 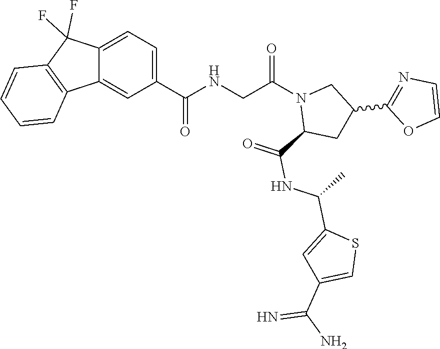 [SMILES: C[C@@H](NC(=O)[C@@H]1CC(c2ncco2)CN1C(=O)CNC(=O)c1ccc2c(c1)-c1ccccc1C2(F)F)c1cc(C(=N)N)cs1] | (S)-N-((R)-1-(4- carbamimidoylthiophen- 2-yl)ethyl)-1-((9,9- difluoro-9H-fluorene-3- carbonyl)glycyl)-4- (oxazol-2-yl)pyrrolidine- 2-carboxamide | *** | 1.34 | 619 |
| 417 | 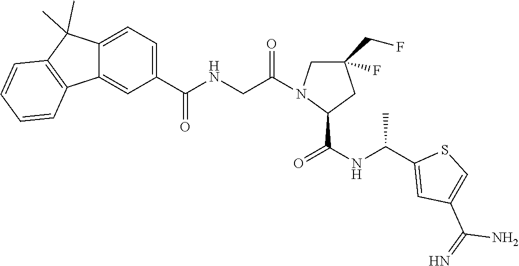 [SMILES: C[C@@H](NC(=O)[C@@H]1C[C@](F)(CF)CN1C(=O)CNC(=O)c1ccc2c(c1)-c1ccccc1C2(C)C)c1cc(C(=N)N)cs1] | (2S,4R)-N-((R)-1-(4- carbamimidoylthiophen- 2-yl)ethyl)-4-fluoro-4- (fluoromethyl)-1-((9- methyl-9H-fluorene-3- carbonyl)glycyl) pyrrolidine-2- carboxamide | *** | 1.39 | 580 |
| 418 | 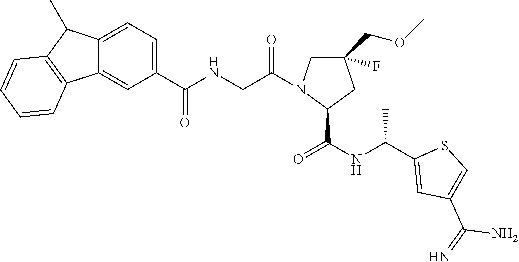 [SMILES: COC[C@@]1(F)C[C@@H](C(=O)N[C@H](C)c2cc(C(=N)N)cs2)N(C(=O)CNC(=O)c2ccc3c(c2)-c2ccccc2C3C)C1] | (2S,4R)-N-((R)-1-(4- carbamimidoylthiophen- 2-yl)ethyl)-4-fluoro-4- (methoxymethyl)-1-((9- methyl-9H-fluorene-3- carbonyl)glycyl) pyrrolidine-2- carboxamide | *** | 1.36 | 592 |
| 419 | 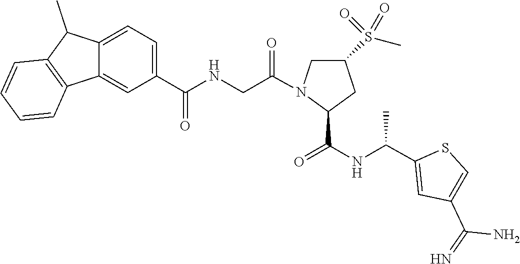 [SMILES: CC1c2ccccc2-c2cc(C(=O)NCC(=O)N3C[C@H](S(C)(=O)=O)C[C@H]3C(=O)N[C@H](C)c3cc(C(=N)N)cs3)ccc21] | (2S,4R)-N-((R)-1-(4- carbamimidoylthiophen- 2-yl)ethyl)-1-((9- methyl-9H-fluorene-3- carbonyl)glycyl)-4- (methylsulfonyl) pyrrolidine-2- carboxamide | *** | 1.28 | 608 |
| 420 | 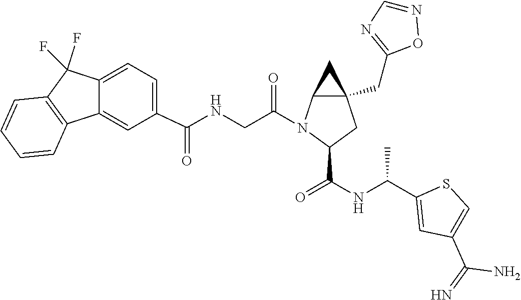 [SMILES: C[C@@H](NC(=O)[C@@H]1C[C@]2(Cc3ncno3)C[C@@H]2N1C(=O)CNC(=O)c1ccc2c(c1)-c1ccccc1C2(F)F)c1cc(C(=N)N)cs1] | (1S,3S,5S)-5-((1,2,4- oxadiazol-5-yl)methyl)- N-((R)-1-(4- carbamimidoylthiophen- 2-yl)ethyl)-2-((9,9- difluoro-9H-fluorene-3- carbonyl)glycyl)-2- azabicyclo[3.1.0]hexane- 3-carboxamide | *** | 1.43 | 646 |
| 421 | 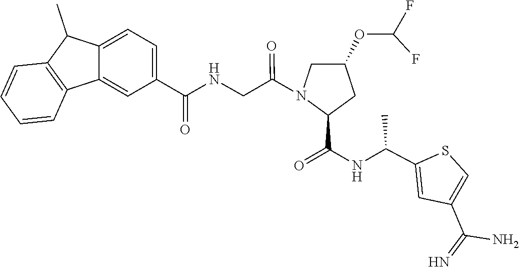 [SMILES: CC1c2ccccc2-c2cc(C(=O)NCC(=O)N3C[C@H](OC(F)F)C[C@H]3C(=O)N[C@H](C)c3cc(C(=N)N)cs3)ccc21] | (2S,4R)-N-((R)-1-(4- carbamimidoylthiophen- 2-yl)ethyl)-4- (difluoromethoxy)-1-((9- methyl-9H-fluorene-3- carbonyl)glycyl) pyrrolidine-2- carboxamide | *** | 1.52 | 596 |
| 422 | 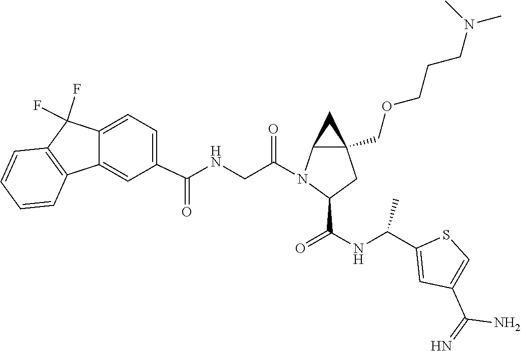 [SMILES: C[C@@H](NC(=O)[C@@H]1C[C@]2(COCCCN(C)C)C[C@@H]2N1C(=O)CNC(=O)c1ccc2c(c1)-c1ccccc1C2(F)F)c1cc(C(=N)N)cs1] | (1S,3S,5R)-N-((R)-1-(4- carbamimidoylthiophen- 2-yl)ethyl)-2-((9,9- difluoro-9H-fluorene-3- carbonyl)glycyl)-5-((3- (dimethylamino) propoxy)methyl)-2- azabicyclo[3.1.0]hexane- 3-carboxamide | *** | 0.96 | 679 |
| 423 | 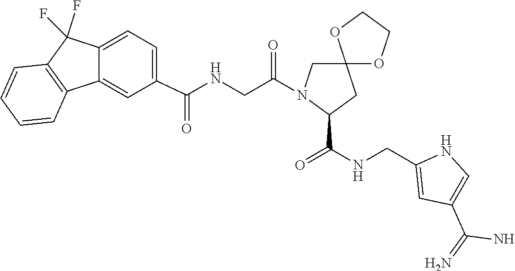 [SMILES: N=C(N)c1c[nH]c(CNC(=O)[C@@H]2CC3(CN2C(=O)CNC(=O)c2ccc4c(c2)-c2ccccc2C4(F)F)OCCO3)c1] | (S)-N-((4- carbamimidoyl-1H- pyrrol-2-yl)methyl)-7- ((9,9-difluoro-9H- fluorene-3- carbonyl)glycyl)-1,4- dioxa-7- azaspiro[4.4]nonane-8- carboxamide | *** | 1.23 | 579 |

### Example 3. Human C1s Enzyme Assay

Human complement C1s enzyme (purified from human serum, Complement Technology, Inc.) at 1.16 nM final concentration was incubated with test compound at various concentrations for 5 min at room temperature in 50 mM Tris, 1 M NaCl, pH 7.5. A synthetic substrate Z-L-Lys-SBzl and DTNB (Ellman's reagent) were added to final concentrations of 100 μM each. Absorbance at 405 nm (A405) was recorded at 30 second intervals for 30 min using a microplate spectrophotometer. IC₅₀ values were calculated by nonlinear regression of complement C1s reaction rates as a function of test compound concentration.

### Example 4. Hemolysis Assay

The hemolysis assay was previously described by Dodds, A. W. and Sim, R. B. (1997); Morgan, B. P. (2000). Prior to the assay, the optimum concentration of Normal Human Serum (NHS) needed to achieve 100% lysis of antibody sensitized sheep erythrocytes (EA) is determined by titration. EA are sheep erythrocytes with rabbit IgM anti-sheep erythrocyte antibodies bound to their surface. In the assay, NHS (Complement Technology) is diluted in GVB++ Buffer (0.1% gelatin, 5 mM Veronal, 145 mM NaCl, 0.025% NaN₃, pH 7.3, 0.15 mM calcium chloride and 0.5 mM magnesium chloride, Complement Technology) and incubated with test compound at various concentrations for 2 min at room temperature. EA (Complement Technology) freshly suspended in GVB++ are added to a final concentration of 1×10⁸ cells/mL and reactions are incubated for 60 min at 37° C. Positive control reactions (100% lysis) consist of GVB++ with NHS and EA but without test compound; negative control reactions (0% lysis) consist of GVB++ with EA only. Samples are centrifuged at 2000 g for 3 min and supernatants collected. Absorbance at 405 nm (A405) is recorded using a microplate spectrophotometer. IC₅₀ values are calculated by nonlinear regression from the percentage of hemolysis as a function of test compound concentration.

This specification has been described with reference to various specific embodiments. However, one of ordinary skill in the art appreciates that various modifications and changes can be made without departing from the scope of the invention as set forth in the claims. Accordingly, the specification is to be regarded in an illustrative rather than a restrictive sense, and all such modifications are intended to be included within the scope of the claims.

We claim:

1. A compound of formula (I): 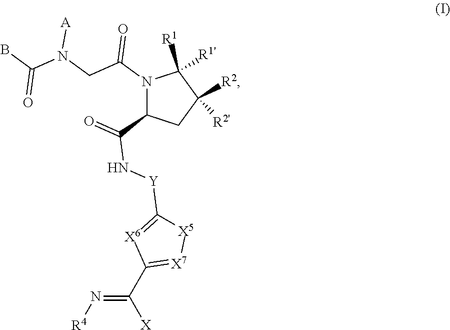
or a pharmaceutically acceptable salt thereof,
wherein
each of R¹, R^{1′}, R², and R^{2′} is independently selected from H; halo; optionally substituted C₁-C₆ alkyl; optionally substituted C₃-C₈ cycloalkyl; optionally substituted C₆-C₁₄ aryl; optionally substituted 5- to 10-membered heterocycle; optionally substituted 5- to 10-membered heteroaryl; optionally substituted C₁-C₆ alkoxy; optionally substituted C₆-C₁₄ aryloxy; SO₂R^{a}, wherein R^{a} is H, C₁-C₆ alkyl, or C₃-C₈ cycloalkyl; and S(O)(NH)R^{b}, wherein R^{b} is H or C₁-C₆ alkyl; wherein at least one of R¹, R^{1′}, R², and R^{2′} is not H; or
R¹ and R², together with the atoms to which each is attached, form optionally substituted C₃-C₈ cycloalkyl; or
R² and R^{2′}, together with the atom to which they are attached, form optionally substituted 5- or 6-membered spirocyclic heterocycle; or
R² and R^{2′} combine to form ═C(R^{c})₂, wherein each R^{c} is independently H or halo;
Y is selected from 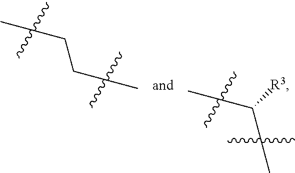
wherein R³ is H, CH₃, CF₃, or CH₂OH;
X is N(R^{d})₂, wherein each R^{d} is independently H, OH, or OC(O)(C₁-C₆ alkyl); and R⁴ is H or C(O)OR^{e}, wherein R^{e} is C₁-C₆ alkyl or C₆-C₁₄ aryl; or
X and R⁴, together with the atoms to which each is attached, form 1H-imidazole-2-yl or (5-aminothiazol-2-yl)thiopheny-2-yl;
A is H or C₁-C₆ alkyl;
B is selected from: 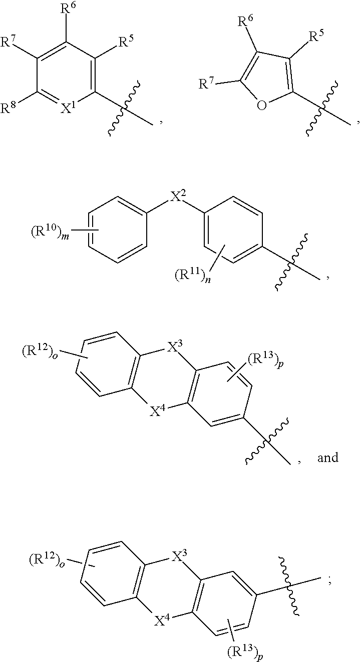
X¹ is CR⁹ or N;
each of R⁵, R⁶, and R⁹ is independently selected from H, halo, and optionally substituted C₁-C₆ alkyl, or
R⁵ and A combine to form optionally substituted C₁-C₂ alkylene;
one of R⁷ and R⁸ is optionally substituted C₆-C₁₄ aryl or optionally substituted 5- to 10-membered heteroaryl; and the other is H, halo, or optionally substituted C₁-C₆ alkyl; or
R⁷ and R⁸, together with the atoms to which each is attached; form optionally substituted 5- to 6-membered heterocycle, optionally substituted 5- to 10-membered heteroaryl, or optionally substituted C₆-C₁₄ aryl; or
R⁶ and R⁹ combine to form (C₂-C₆alkylene)(C₆-C₁₄arylene)(C₂-C₆alkylene), and each of R₅, R₇, and R₈ is H;
X² is O or C(R^{f})₂, wherein each R^{f} is halo;
m is selected from 0, 1, 2, 3, 4, and 5;
n is selected from 0, 1, 2, 3, and 4;
each R¹⁰ and R¹¹ is independently halo, CN, optionally substituted C₁-C₆ alkyl, optionally substituted C₁-C₆ alkoxy, or optionally substituted C₃-C₈ cycloalkyl;
each of X³ and X⁴ is independently a bond; O; S; C(R^{g})₂, wherein each R^{g} is independently H, OH, halo, optionally substituted C₁-C₆ alkyl, or optionally substituted C₁-C₆ alkoxy, or both R^{g} combine to form oxo; NR^{h}, wherein R^{h} is H or C₁-C₆ alkyl; or SO₂;
X⁵ is S, and X⁶ and X⁷ are both CH; or
X⁶ is S, and X⁵ and X⁷ are both CH; or
X⁷ is S, and X⁵ and X⁶ are both CH;
o is selected from 0, 1, 2, 3, and 4;
p is selected from 0, 1, 2, and 3; and
each R¹² and R¹³ is independently halo, optionally substituted C₁-C₆ alkyl, optionally substituted C₁-C₆ alkoxy, or optionally substituted C₃-C₈ cycloalkyl, provided that:
when B is 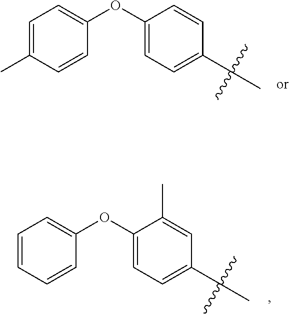 [SMILES: *c1ccc(Oc2ccc(C)cc2)cc1 ; *c1ccc(Oc2ccccc2)c(C)c1]
R³ is CH₃, CF₃, or CH₂OH;
when m is 0, n is 1, 2, 3, or 4; and
when n is 0, m is 1, 2, 3, 4, or 5.

2. The compound of claim 1, wherein the compound is a compound of formula (II): 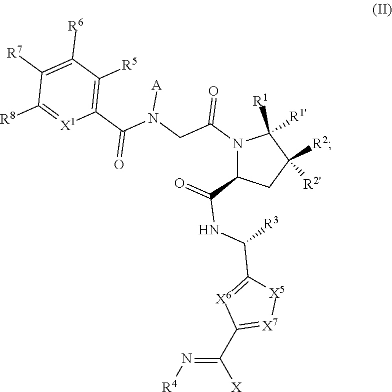
or a pharmaceutically acceptable salt thereof.

3-7. (canceled)

8. The compound of claim 2, wherein R⁷ is optionally substituted C₆-C₁₄ aryl or optionally substituted 5- to 10-membered heteroaryl.

9-10. (canceled)

11. The compound of claim 8, wherein R⁷ is:

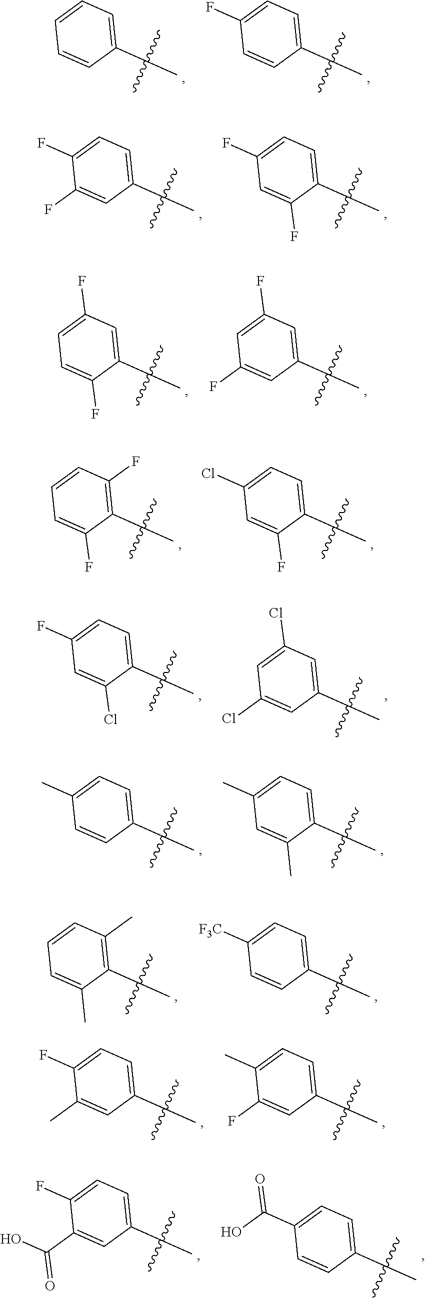

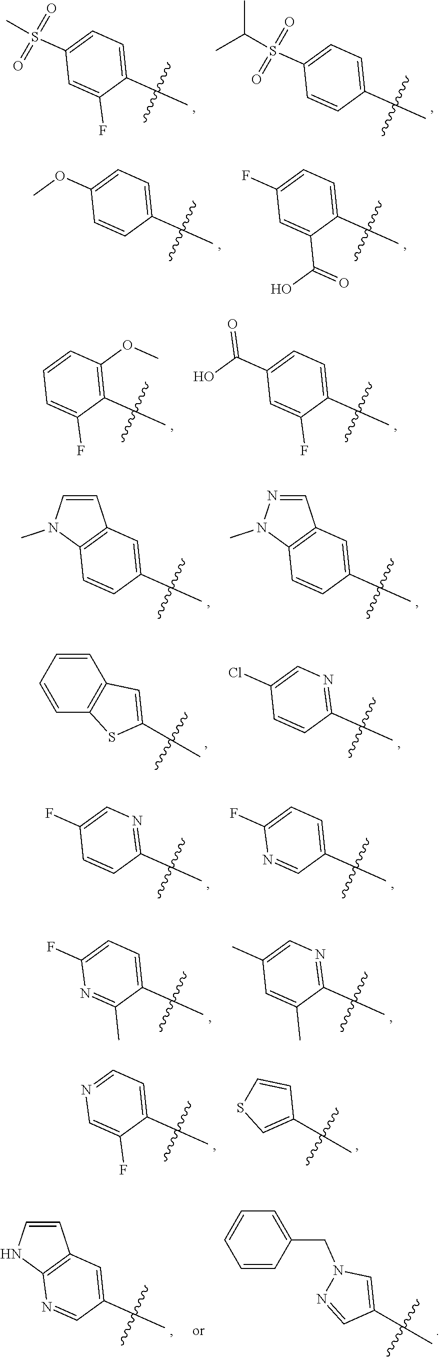

12-13. (canceled)

14. The compound of claim 2, wherein:
R⁸ is H or optionally substituted C₁-C₆ alkyl, or halo; and/or
R⁶ is H; and/or
R⁵ is H or halo.

15-26. (canceled)

27. The compound of claim 1, wherein the compound is a compound of formula (III): 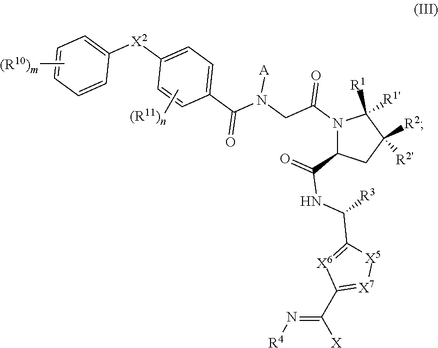
or a pharmaceutically acceptable salt thereof.

28. (canceled)

29. The compound of claim 27, wherein X² is O or CF₂.

30-44. (canceled)

45. The compound of claim 1, wherein the compound is a compound of formula (IV) or (V): 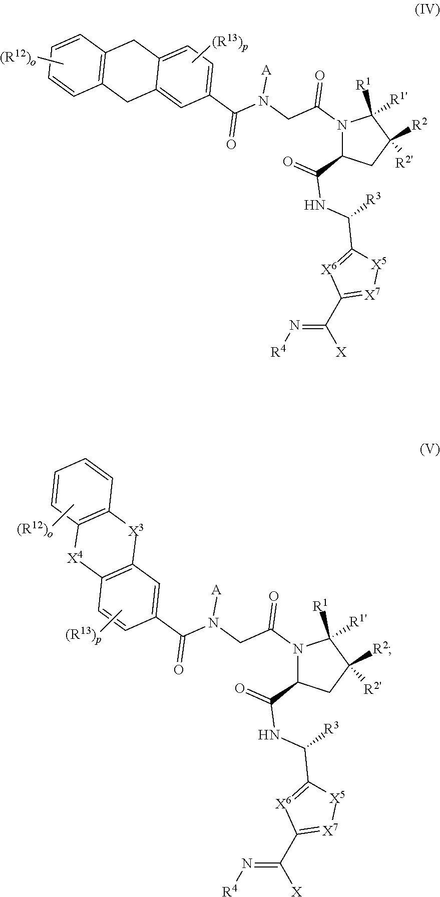
or a pharmaceutically acceptable salt thereof.

46-55. (canceled)

56. The compound of any claim 45, wherein X³ is O, S, SO₂, CF₂, CH₂, CHCH₃, C(CH₃)₂, C(O), CHF, CHOH, CHCHF₂, CHOCHF₂, CHOCH₃, or NH.

57-60. (canceled)

61. The compound of claim 45, wherein X⁴ is a bond, O, or S.

62. (canceled)

63. The compound of claim 1, wherein:
R¹ and R², together with the atoms to which each is attached, combine to form optionally substituted C₃-C₈ cycloalkyl; and/or
R^{1′} is H; and/or
R^{2′} is optionally substituted C₁-C₆ alkyl.

64. The compound of claim 63, wherein R¹ and R², together with the atoms to which each is attached, combine to form cyclopropyl, and/or R^{2′} is CH₃, CH₂OH, CH₂OCH₃, CH₂CF₃, CH₂O(CH₂)₂NH₂, CH₂O(CH₂)₂N(CH₃)₂, CH₂O(CH₂)₃NH₂, CH₂O(CH₂)₃N(CH₃)₂, CH₂O(CH₂)₄NH₂, CH₂O(CH₂)₄N(CH₃)₂, CH₂NH₂, CH₂N(CH₃)₂, CH₂NH(CH₂)₂NHC(O)CH₃,

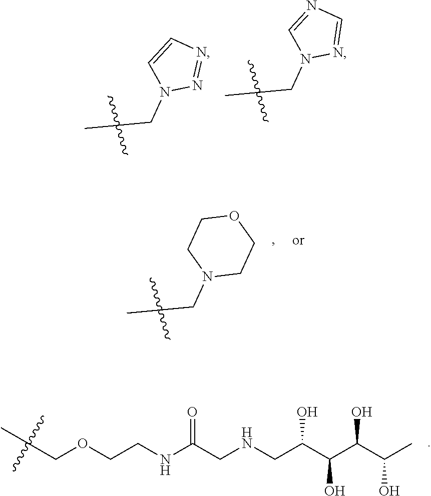 [SMILES: *Cn1ccnn1 ; *Cn1cncn1 ; *CN1CCOCC1 ; *COCCNC(=O)CNC[C@H](O)[C@H](O)[C@@H](O)[C@H](C)O]

65-68. (canceled)

69. The compound of claim 1, wherein:
R² and R^{2′}, together with the atom to which they are attached, form optionally substituted 5- or 6-membered spirocyclic heterocycle or R² and R^{2′} combine to form ═C(R^{c})₂; and/or
R¹ is H; and/or
R^{1′} is H.

70. The compound of claim 1, wherein R² and R^{2′}, together with the atom to which they are attached, form 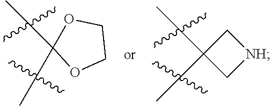 [SMILES: *C1(*)OCCO1 ; *C1(*)CNC1]
or R² and R^{2′} combine to form ═CH₂ or ═CF₂.

71. The compound of any claim 1, wherein:
R² is H; and/or
R^{2′} is CH₃, CH₂OH, CH₂OCH₃, CF₃, phenyl, OCH₃, OCHF₂, OCF₃, F, SO₂CH₃, SO₂CH₂CH₃, 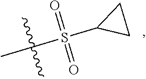 [SMILES: *S(=O)(=O)C1CC1]
S(O)(NH)CH₃, 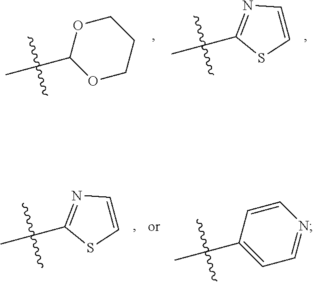 [SMILES: *C1OCCCO1 ; *c1nccs1 ; *c1nccs1 ; *c1ccncc1]
and/or
R¹ is H; and/or
R^{1′} is H.

72. (canceled)

73. The compound of claim 71, wherein:
R² is CH₂F, CF₃, CH₂OH, CH₂OCH₃, CH₂O(CH₂)₅COOH, CH₂O(CH₂)₇COOH, CH₂O(CH₂)COOH, 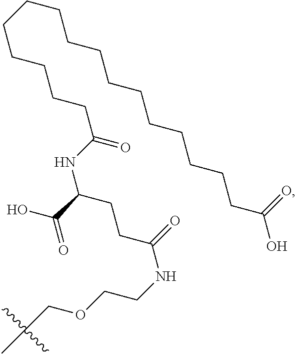 [SMILES: *COCCNC(=O)CC[C@H](NC(=O)CCCCCCCCCCCCCCCCC(=O)O)C(=O)O]
CH₂NH₂, CH₂N(CH₃)₂, 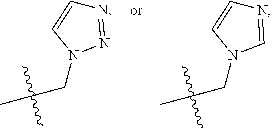 [SMILES: *Cn1ccnn1 ; *Cn1ccnc1]
CH₃, 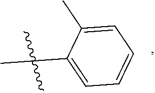 [SMILES: *c1ccccc1C]
cyclohexyl, 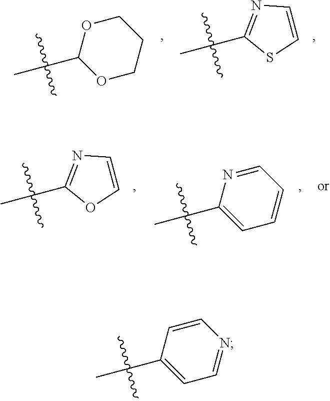 [SMILES: *C1OCCCO1 ; *c1nccs1 ; *c1ncco1 ; *c1ccccn1 ; *c1ccncc1]
and/or
R^{2′} is H; and/or
R¹ is H; and/or
R^{1′} is H.

74-104. (canceled)

105. The compound of claim 1, wherein:
Y is 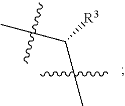
and/or
R³ is CH₃ or CF₃; and/or
X is NH₂, NHOC(O)(C₁-C₆ alkyl), or NHOH; and/or R⁴ is H, C(O)OCH₃, C(O)OCH(CH₃)₂, C(O)O(CH₂)₅CH₃, or C(O)OC₆H₅; and/or
X⁵ is S, and X⁶ and X⁷ are both CH; and/or
A is H.

106-117. (canceled)

118. A compound selected from: 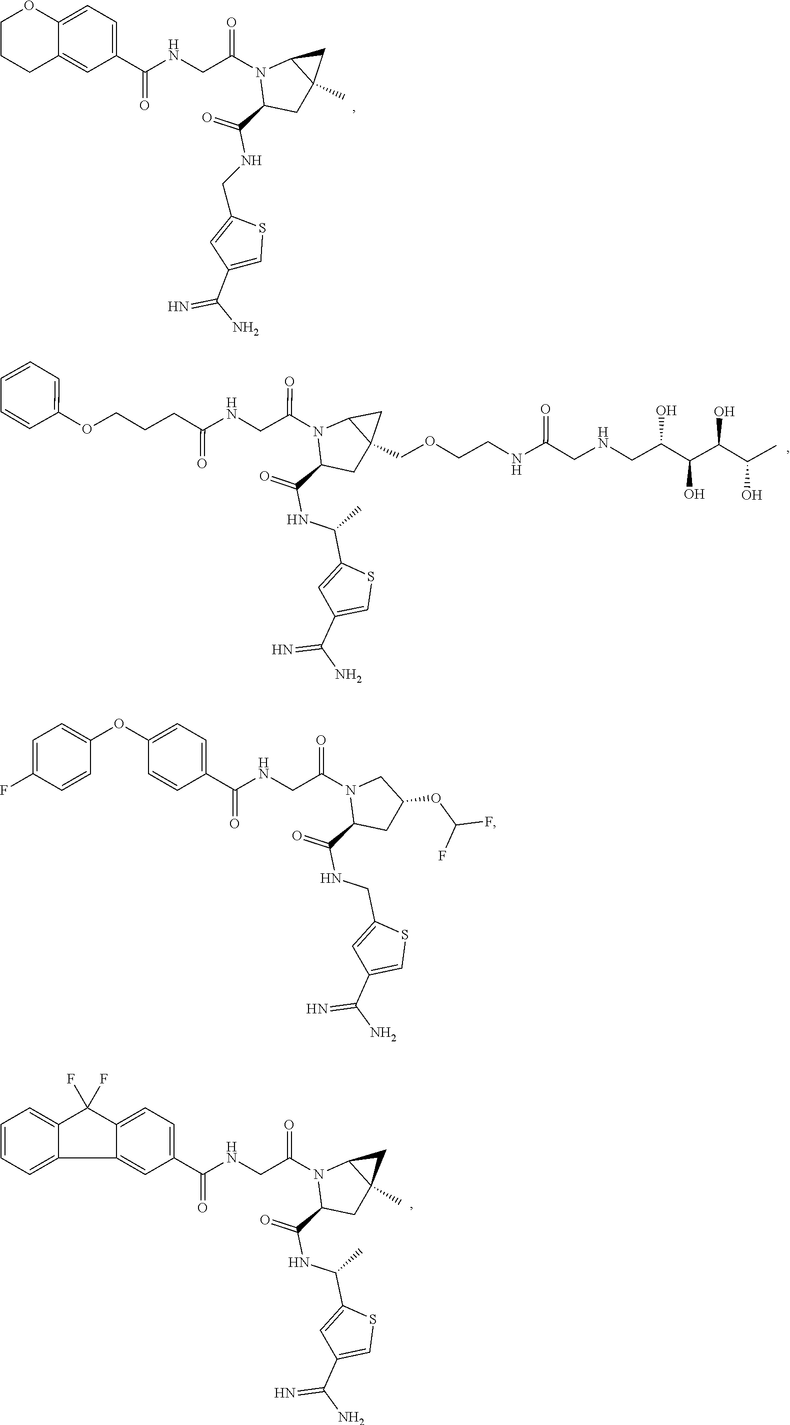 [SMILES: C[C@@]12C[C@@H]1N(C(=O)CNC(=O)c1ccc3c(c1)CCCO3)[C@H](C(=O)NCc1cc(C(=N)N)cs1)C2 ; C[C@H](O)[C@H](O)[C@@H](O)[C@@H](O)CNCC(=O)NCCOC[C@@]12CC1N(C(=O)CNC(=O)CCCOc1ccccc1)[C@H](C(=O)N[C@H](C)c1cc(C(=N)N)cs1)C2 ; N=C(N)c1csc(CNC(=O)[C@@H]2C[C@@H](OC(F)F)CN2C(=O)CNC(=O)c2ccc(Oc3ccc(F)cc3)cc2)c1 ; C[C@@H](NC(=O)[C@@H]1C[C@]2(C)C[C@@H]2N1C(=O)CNC(=O)c1ccc2c(c1)-c1ccccc1C2(F)F)c1cc(C(=N)N)cs1] 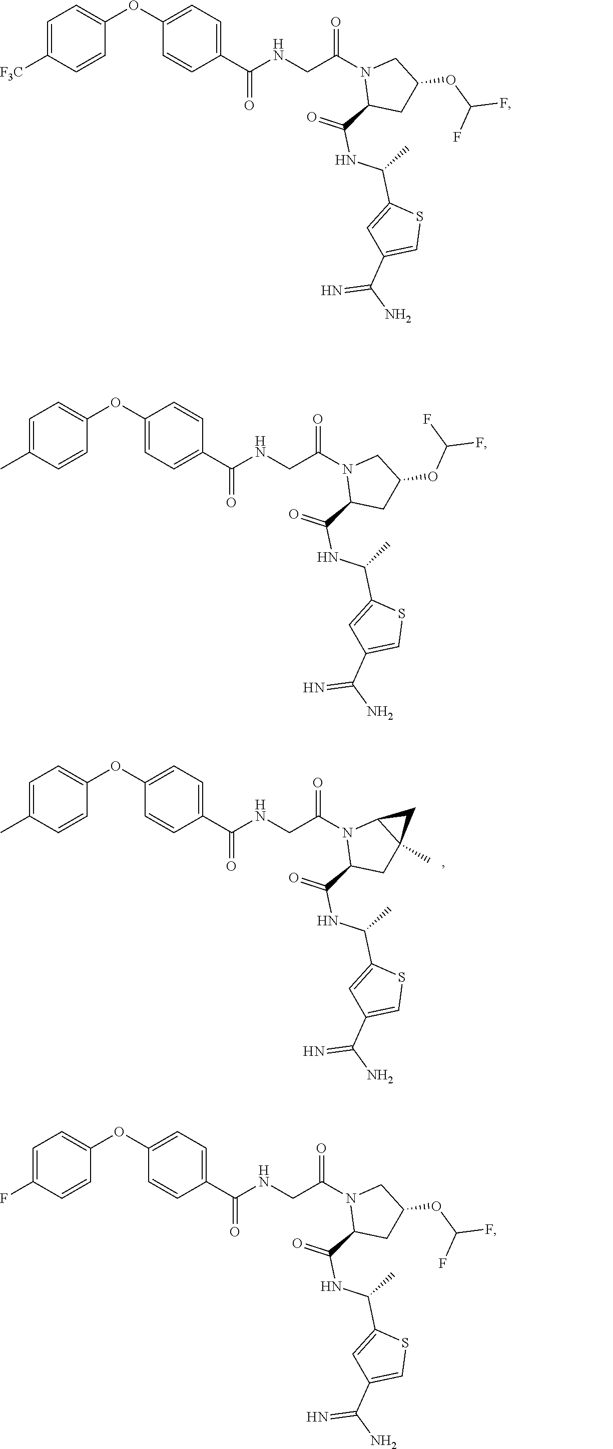 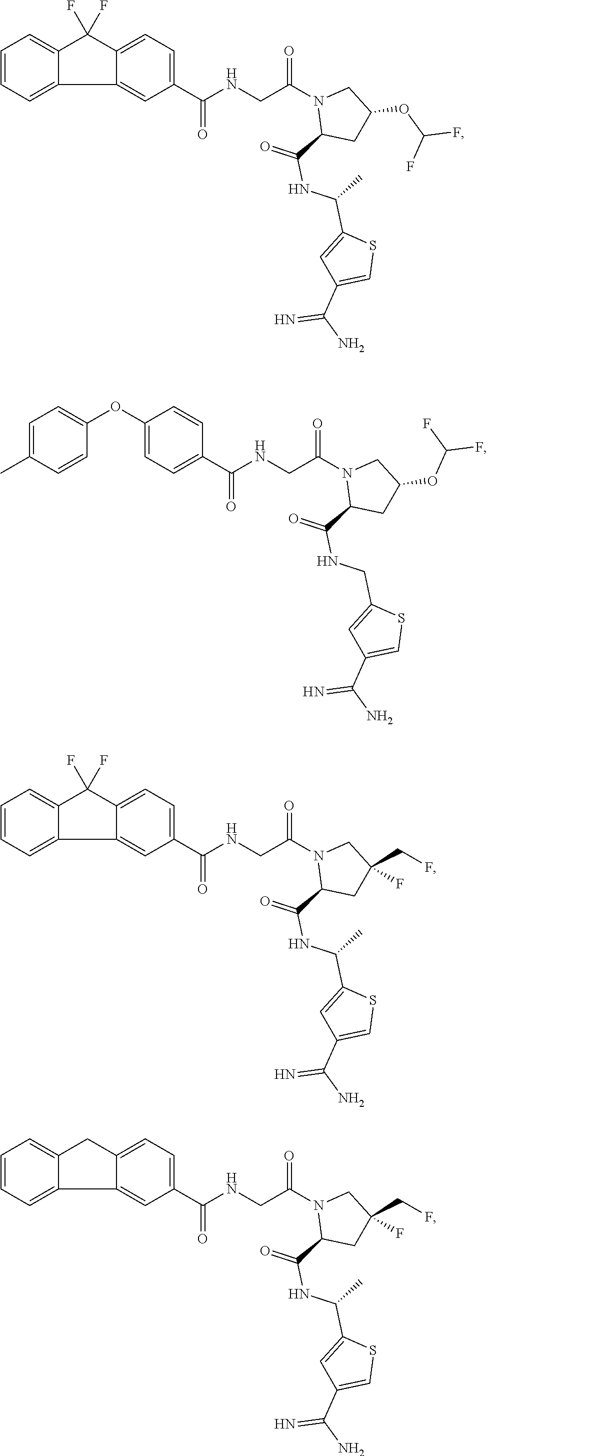 [SMILES: C[C@@H](NC(=O)[C@@H]1C[C@@H](OC(F)F)CN1C(=O)CNC(=O)c1ccc2c(c1)-c1ccccc1C2(F)F)c1cc(C(=N)N)cs1 ; Cc1ccc(Oc2ccc(C(=O)NCC(=O)N3C[C@H](OC(F)F)C[C@H]3C(=O)NCc3cc(C(=N)N)cs3)cc2)cc1 ; C[C@@H](NC(=O)[C@@H]1C[C@](F)(CF)CN1C(=O)CNC(=O)c1ccc2c(c1)-c1ccccc1C2(F)F)c1cc(C(=N)N)cs1 ; C[C@@H](NC(=O)[C@@H]1C[C@](F)(CF)CN1C(=O)CNC(=O)c1ccc2c(c1)-c1ccccc1C2)c1cc(C(=N)N)cs1] 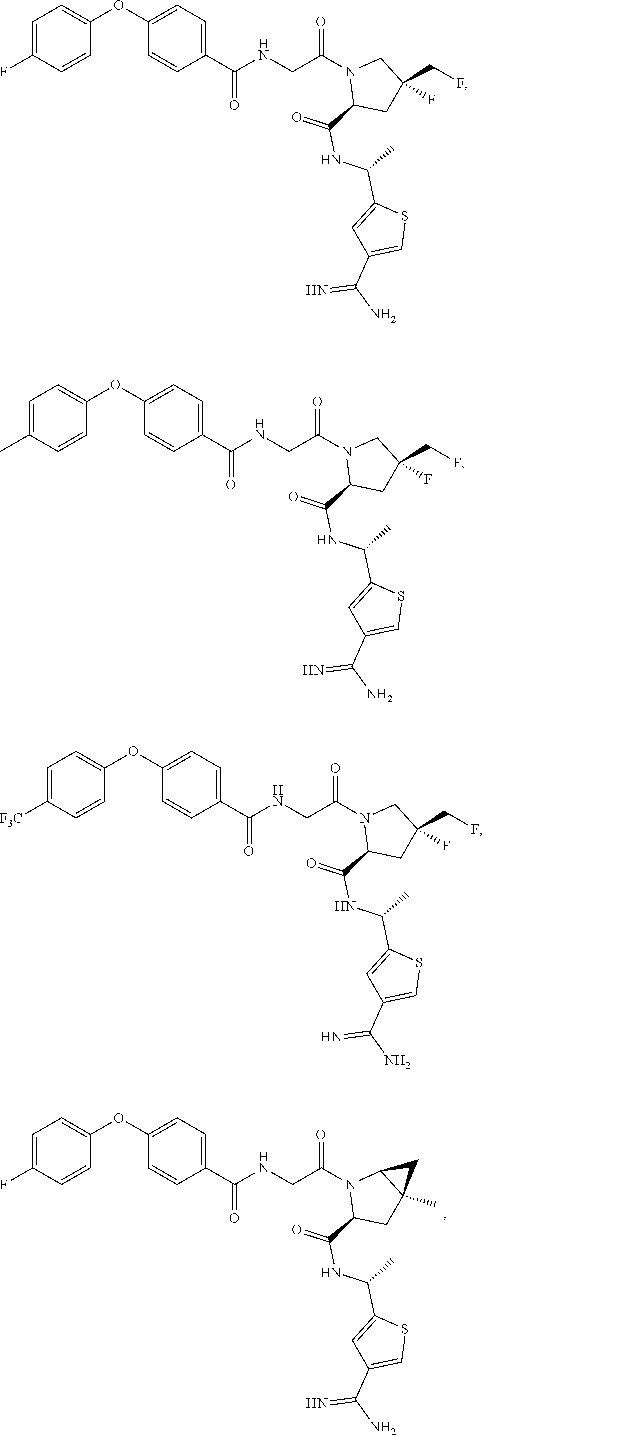 [SMILES: C[C@@H](NC(=O)[C@@H]1C[C@](F)(CF)CN1C(=O)CNC(=O)c1ccc(Oc2ccc(F)cc2)cc1)c1cc(C(=N)N)cs1 ; Cc1ccc(Oc2ccc(C(=O)NCC(=O)N3C[C@@](F)(CF)C[C@H]3C(=O)N[C@H](C)c3cc(C(=N)N)cs3)cc2)cc1 ; C[C@@H](NC(=O)[C@@H]1C[C@](F)(CF)CN1C(=O)CNC(=O)c1ccc(Oc2ccc(C(F)(F)F)cc2)cc1)c1cc(C(=N)N)cs1 ; C[C@@H](NC(=O)[C@@H]1C[C@]2(C)C[C@@H]2N1C(=O)CNC(=O)c1ccc(Oc2ccc(F)cc2)cc1)c1cc(C(=N)N)cs1] 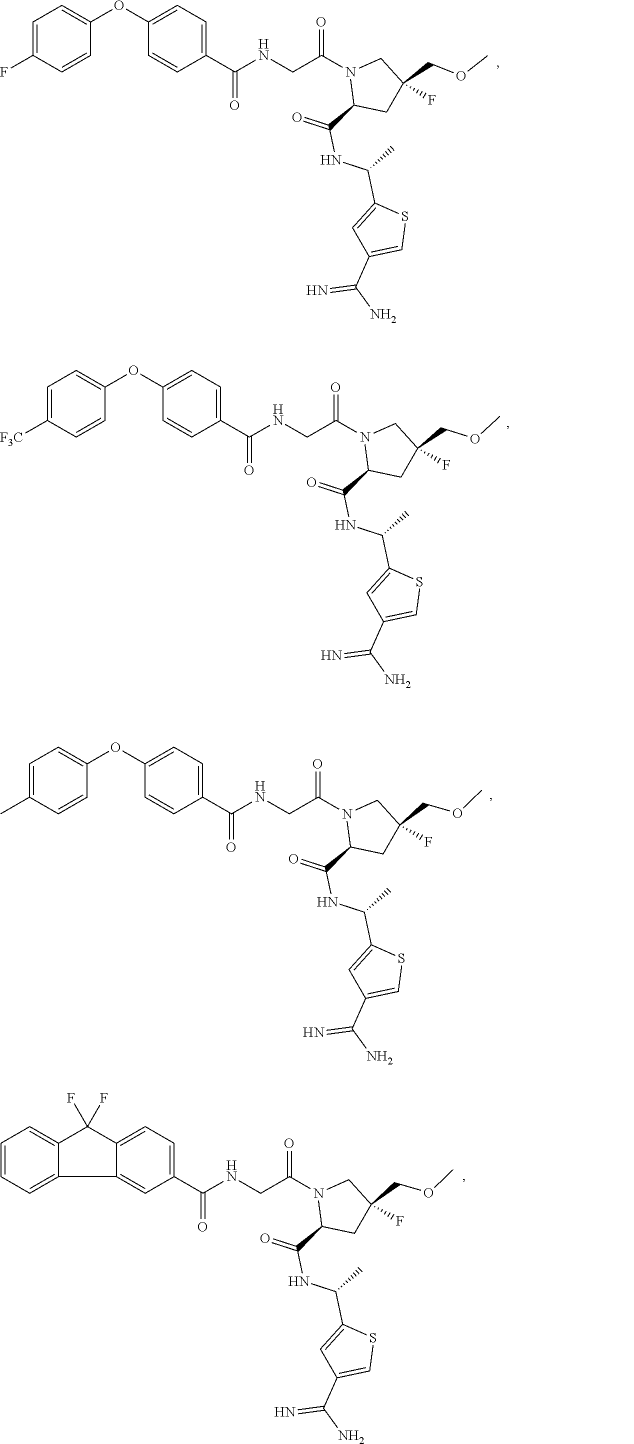 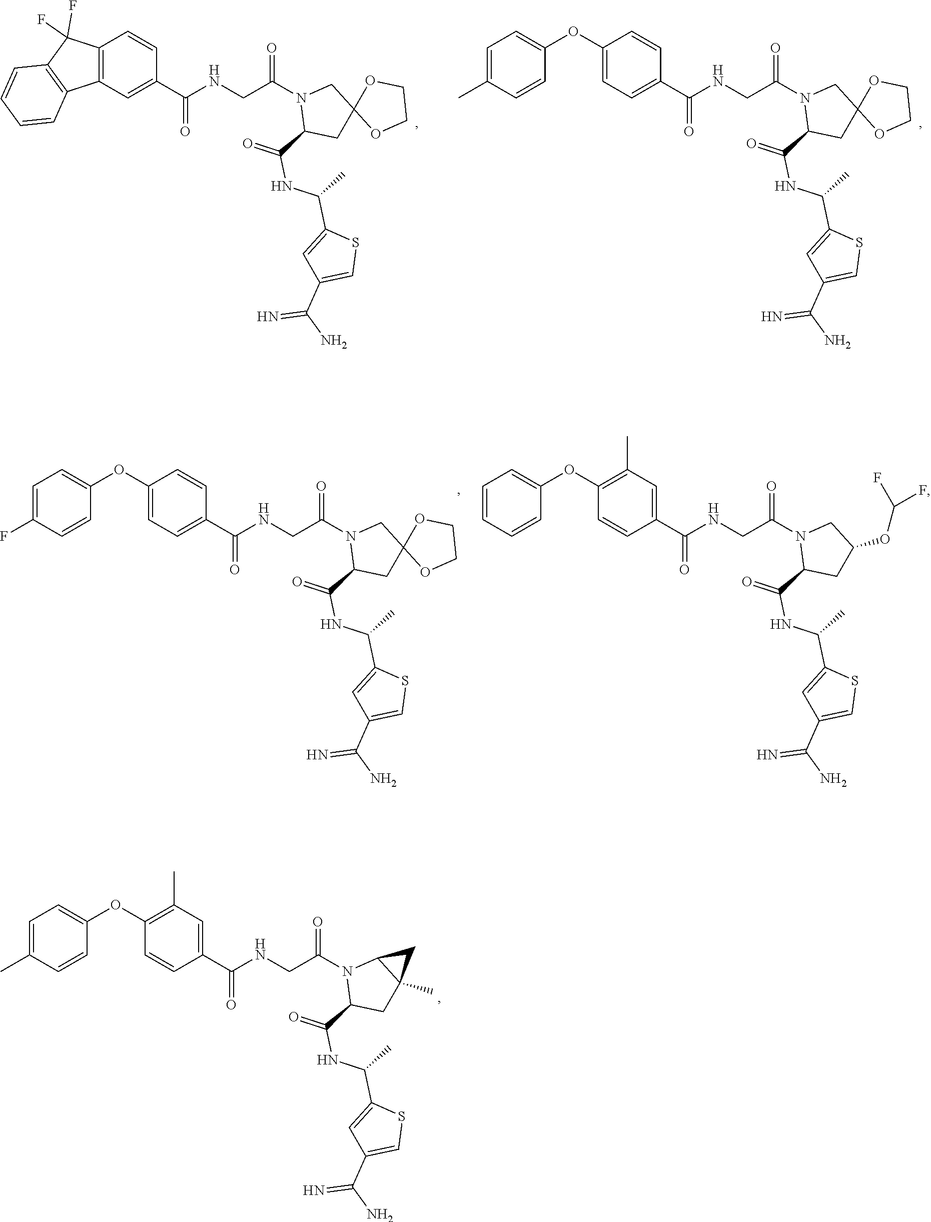 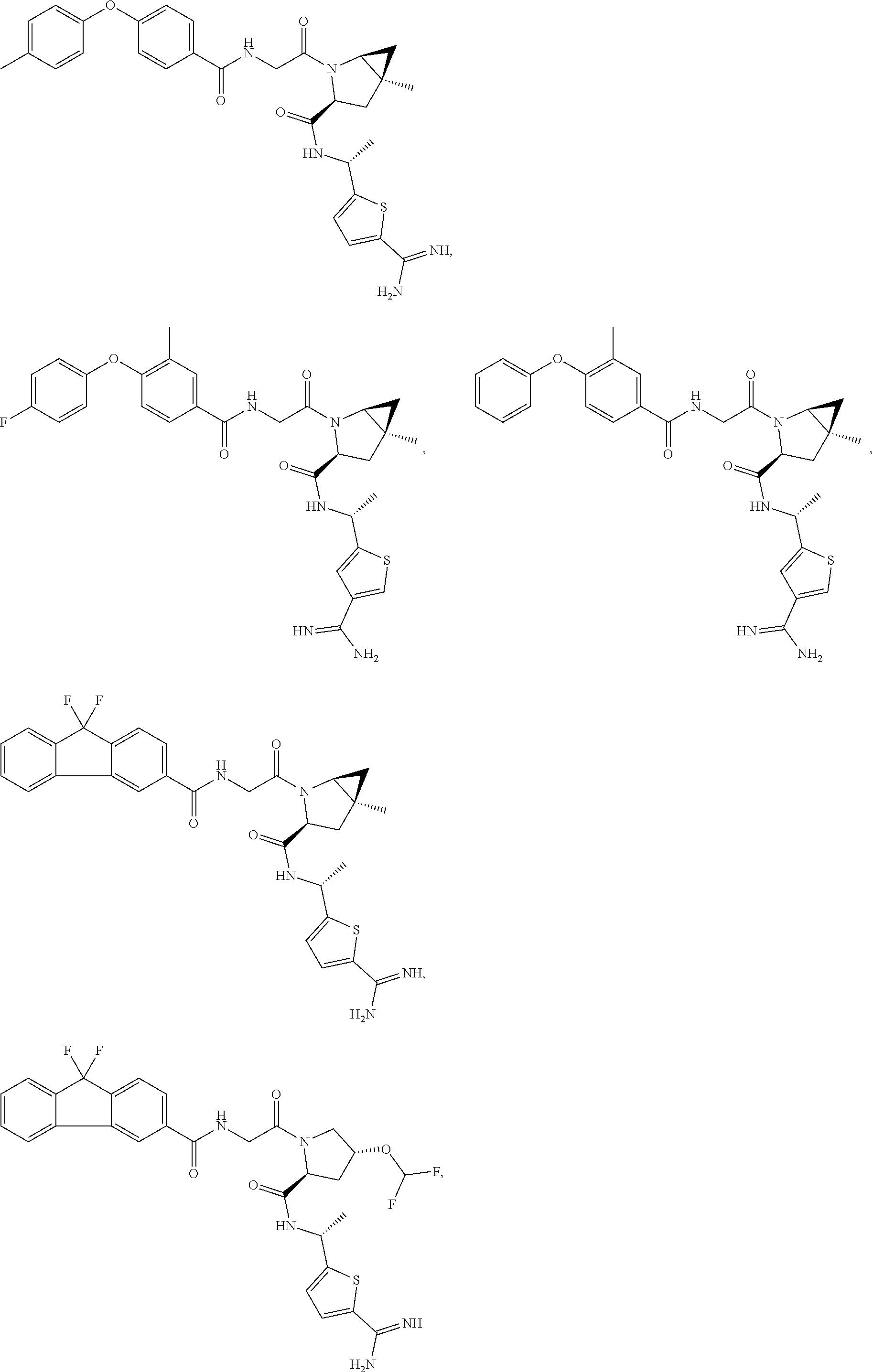 [SMILES: Cc1ccc(Oc2ccc(C(=O)NCC(=O)N3[C@H]4C[C@@]4(C)C[C@H]3C(=O)N[C@H](C)c3ccc(C(=N)N)s3)cc2)cc1 ; Cc1cc(C(=O)NCC(=O)N2[C@H]3C[C@@]3(C)C[C@H]2C(=O)N[C@H](C)c2cc(C(=N)N)cs2)ccc1Oc1ccc(F)cc1 ; Cc1cc(C(=O)NCC(=O)N2[C@H]3C[C@@]3(C)C[C@H]2C(=O)N[C@H](C)c2cc(C(=N)N)cs2)ccc1Oc1ccccc1 ; C[C@@H](NC(=O)[C@@H]1C[C@]2(C)C[C@@H]2N1C(=O)CNC(=O)c1ccc2c(c1)-c1ccccc1C2(F)F)c1ccc(C(=N)N)s1 ; C[C@@H](NC(=O)[C@@H]1C[C@@H](OC(F)F)CN1C(=O)CNC(=O)c1ccc2c(c1)-c1ccccc1C2(F)F)c1ccc(C(=N)N)s1] 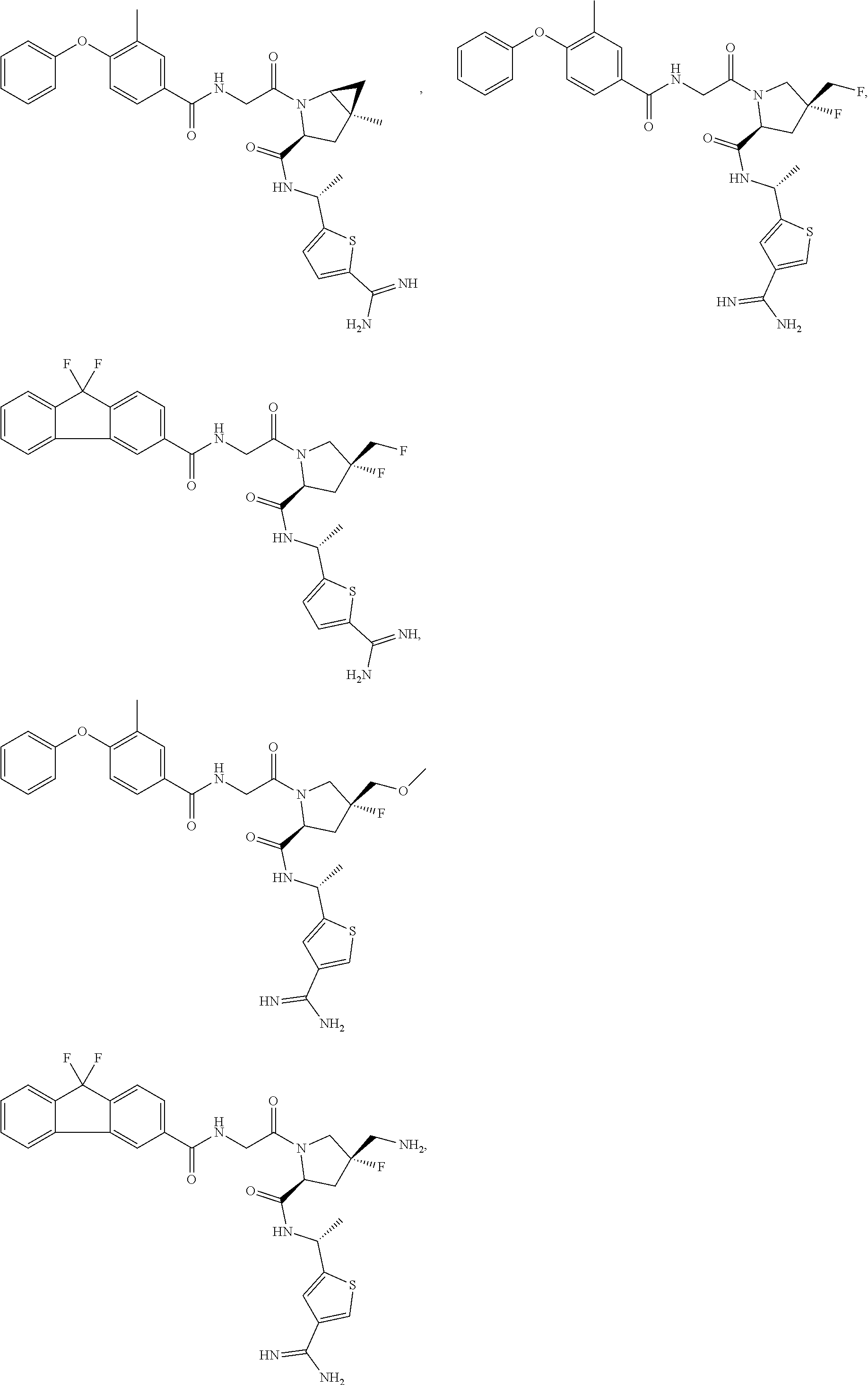 [SMILES: Cc1cc(C(=O)NCC(=O)N2[C@H]3C[C@@]3(C)C[C@H]2C(=O)N[C@H](C)c2ccc(C(=N)N)s2)ccc1Oc1ccccc1 ; Cc1cc(C(=O)NCC(=O)N2C[C@@](F)(CF)C[C@H]2C(=O)N[C@H](C)c2cc(C(=N)N)cs2)ccc1Oc1ccccc1 ; C[C@@H](NC(=O)[C@@H]1C[C@](F)(CF)CN1C(=O)CNC(=O)c1ccc2c(c1)-c1ccccc1C2(F)F)c1ccc(C(=N)N)s1 ; COC[C@@]1(F)C[C@@H](C(=O)N[C@H](C)c2cc(C(=N)N)cs2)N(C(=O)CNC(=O)c2ccc(Oc3ccccc3)c(C)c2)C1 ; C[C@@H](NC(=O)[C@@H]1C[C@](F)(CN)CN1C(=O)CNC(=O)c1ccc2c(c1)-c1ccccc1C2(F)F)c1cc(C(=N)N)cs1] 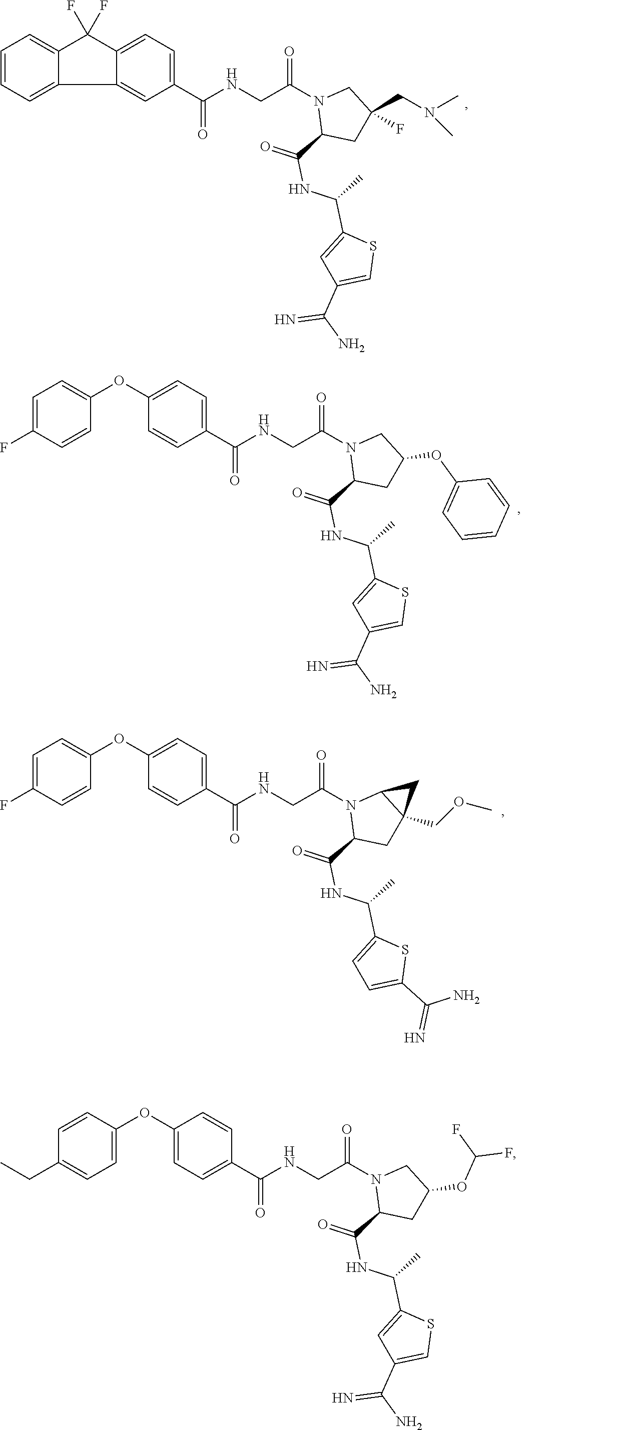 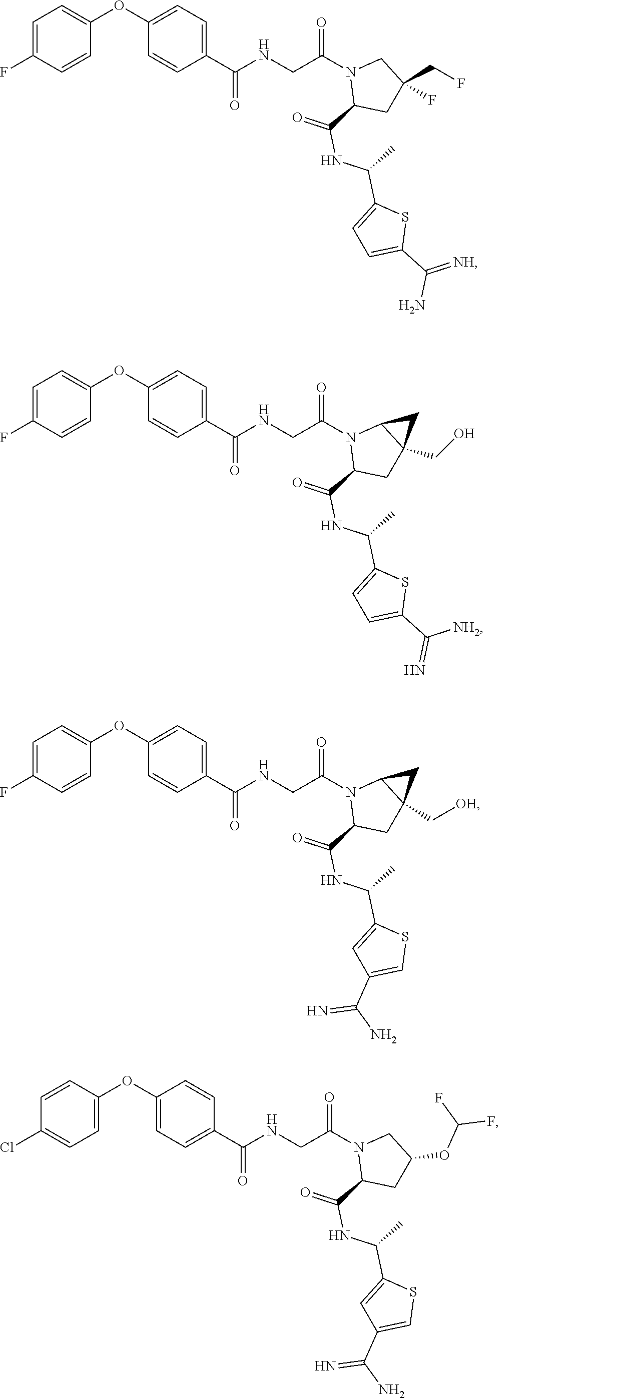 [SMILES: C[C@@H](NC(=O)[C@@H]1C[C@](F)(CF)CN1C(=O)CNC(=O)c1ccc(Oc2ccc(F)cc2)cc1)c1ccc(C(=N)N)s1 ; C[C@@H](NC(=O)[C@@H]1C[C@]2(CO)C[C@@H]2N1C(=O)CNC(=O)c1ccc(Oc2ccc(F)cc2)cc1)c1ccc(C(=N)N)s1 ; C[C@@H](NC(=O)[C@@H]1C[C@]2(CO)C[C@@H]2N1C(=O)CNC(=O)c1ccc(Oc2ccc(F)cc2)cc1)c1cc(C(=N)N)cs1 ; C[C@@H](NC(=O)[C@@H]1C[C@@H](OC(F)F)CN1C(=O)CNC(=O)c1ccc(Oc2ccc(Cl)cc2)cc1)c1cc(C(=N)N)cs1] 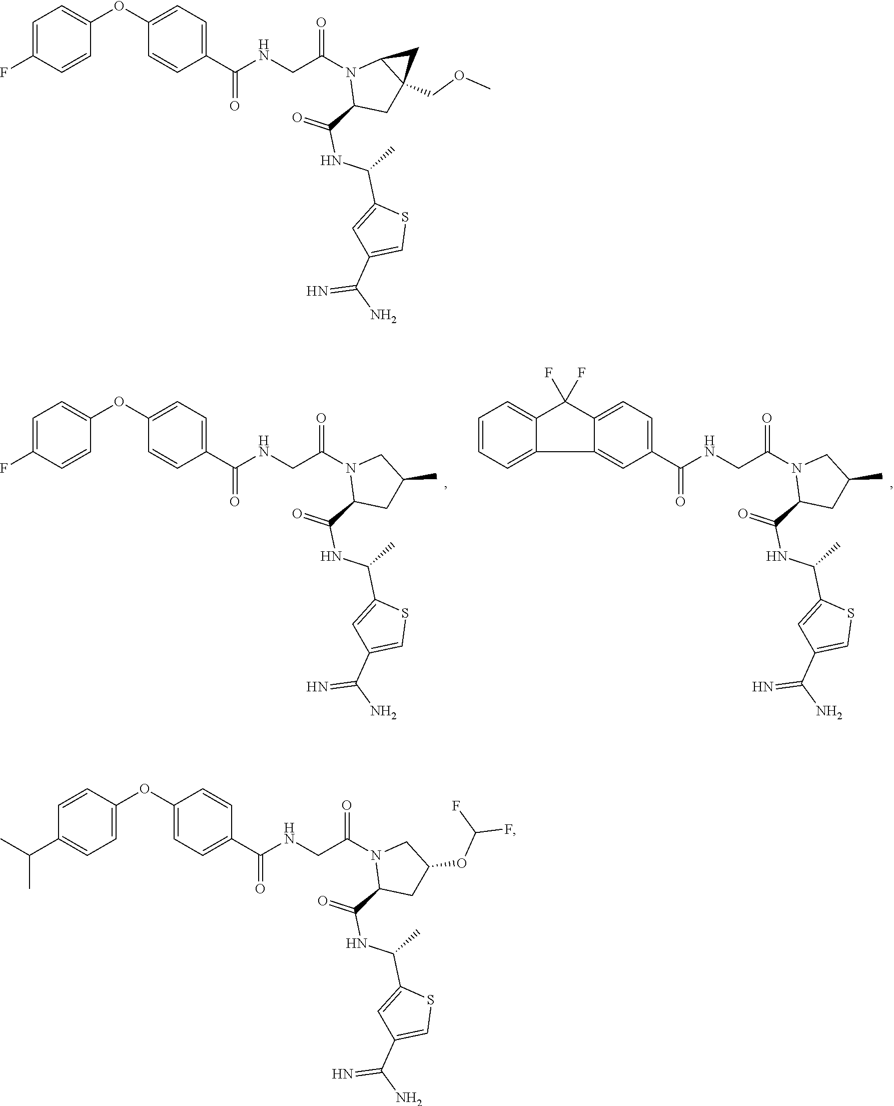 [SMILES: COC[C@@]12C[C@@H]1N(C(=O)CNC(=O)c1ccc(Oc3ccc(F)cc3)cc1)[C@H](C(=O)N[C@H](C)c1cc(C(=N)N)cs1)C2 ; C[C@H]1C[C@@H](C(=O)N[C@H](C)c2cc(C(=N)N)cs2)N(C(=O)CNC(=O)c2ccc(Oc3ccc(F)cc3)cc2)C1 ; C[C@H]1C[C@@H](C(=O)N[C@H](C)c2cc(C(=N)N)cs2)N(C(=O)CNC(=O)c2ccc3c(c2)-c2ccccc2C3(F)F)C1 ; CC(C)c1ccc(Oc2ccc(C(=O)NCC(=O)N3C[C@H](OC(F)F)C[C@H]3C(=O)N[C@H](C)c3cc(C(=N)N)cs3)cc2)cc1] 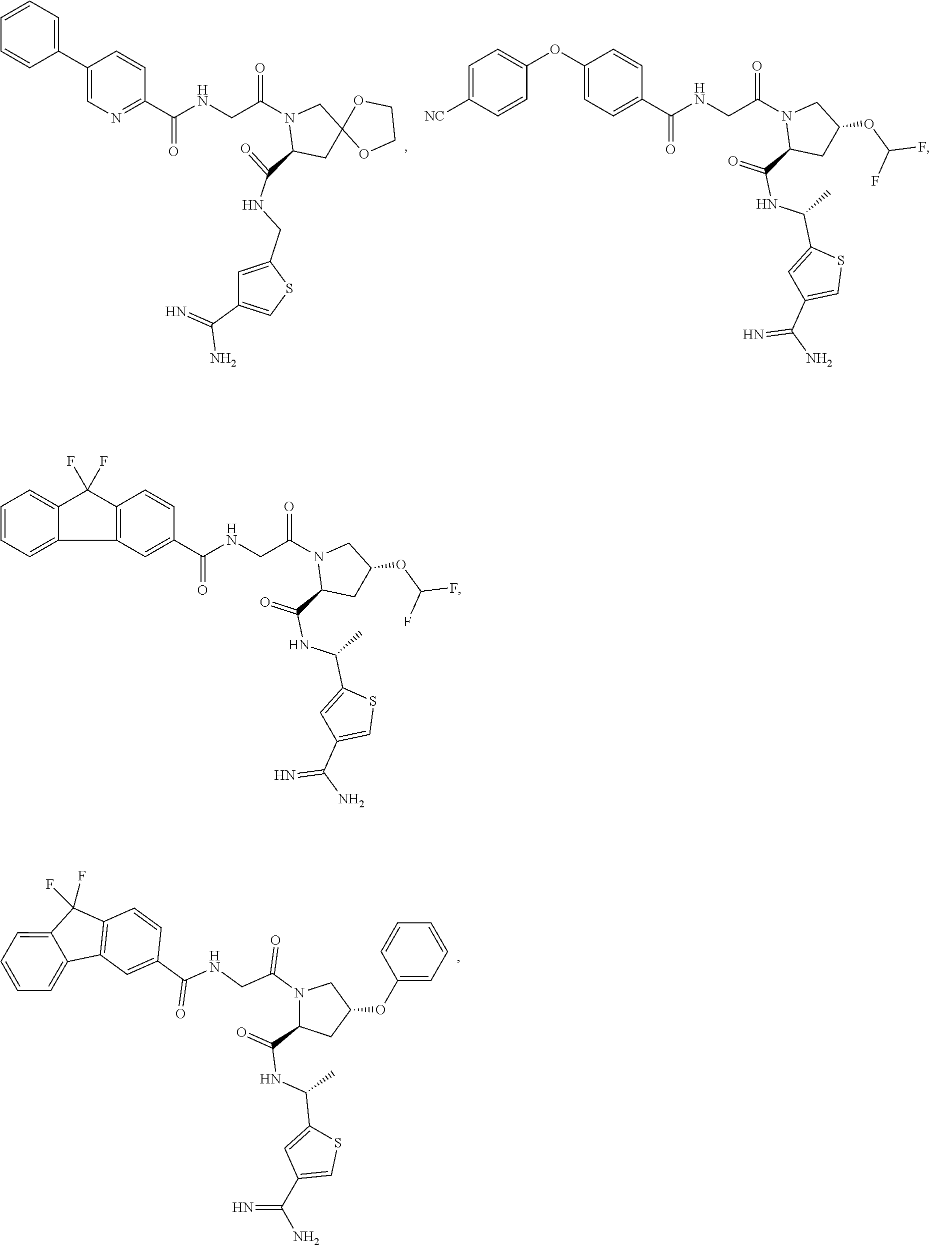 [SMILES: N=C(N)c1csc(CNC(=O)[C@@H]2CC3(CN2C(=O)CNC(=O)c2ccc(-c4ccccc4)cn2)OCCO3)c1 ; C[C@@H](NC(=O)[C@@H]1C[C@@H](OC(F)F)CN1C(=O)CNC(=O)c1ccc(Oc2ccc(C#N)cc2)cc1)c1cc(C(=N)N)cs1 ; C[C@@H](NC(=O)[C@@H]1C[C@@H](OC(F)F)CN1C(=O)CNC(=O)c1ccc2c(c1)-c1ccccc1C2(F)F)c1cc(C(=N)N)cs1 ; C[C@@H](NC(=O)[C@@H]1C[C@@H](Oc2ccccc2)CN1C(=O)CNC(=O)c1ccc2c(c1)-c1ccccc1C2(F)F)c1cc(C(=N)N)cs1] 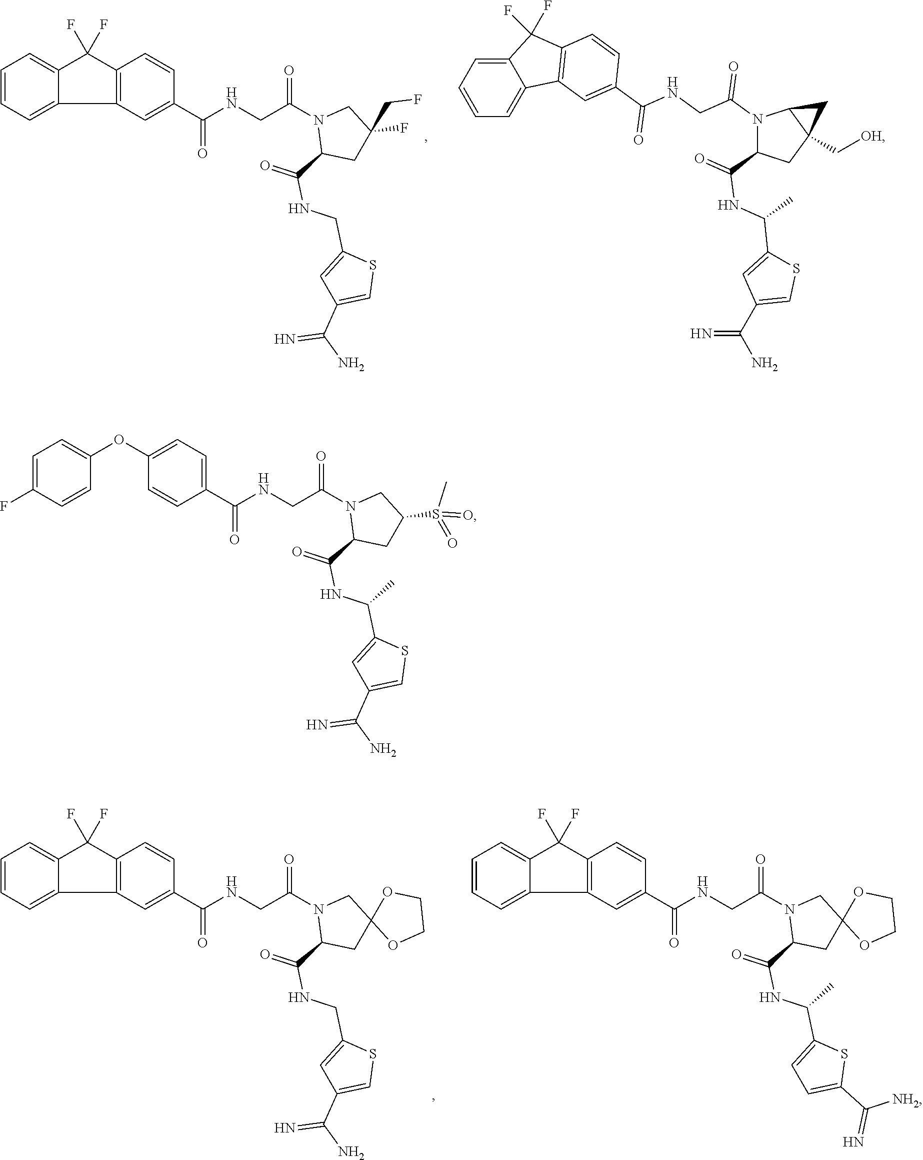 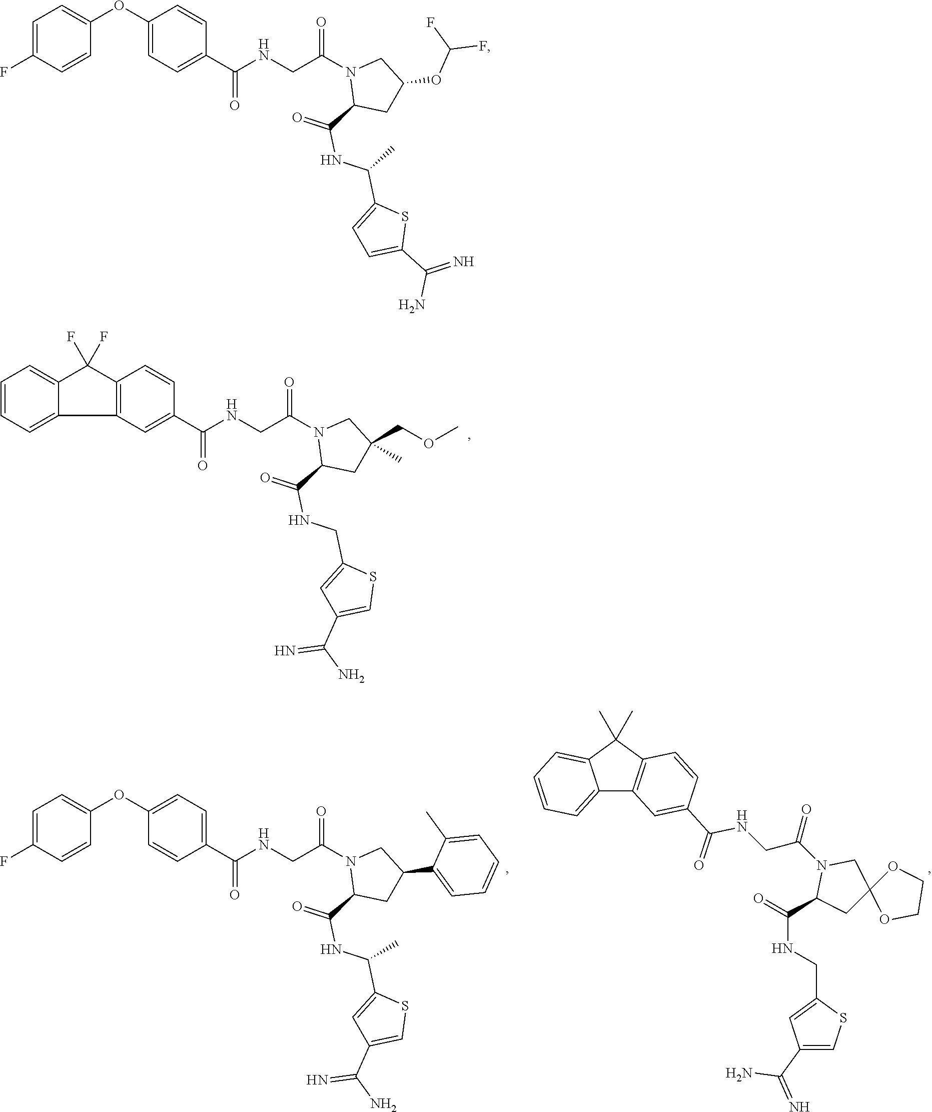 [SMILES: C[C@@H](NC(=O)[C@@H]1C[C@@H](OC(F)F)CN1C(=O)CNC(=O)c1ccc(Oc2ccc(F)cc2)cc1)c1ccc(C(=N)N)s1 ; COC[C@@]1(C)C[C@@H](C(=O)NCc2cc(C(=N)N)cs2)N(C(=O)CNC(=O)c2ccc3c(c2)-c2ccccc2C3(F)F)C1 ; Cc1ccccc1[C@H]1C[C@@H](C(=O)N[C@H](C)c2cc(C(=N)N)cs2)N(C(=O)CNC(=O)c2ccc(Oc3ccc(F)cc3)cc2)C1 ; CC1(C)c2ccccc2-c2cc(C(=O)NCC(=O)N3CC4(C[C@H]3C(=O)NCc3cc(C(=N)N)cs3)OCCO4)ccc21] 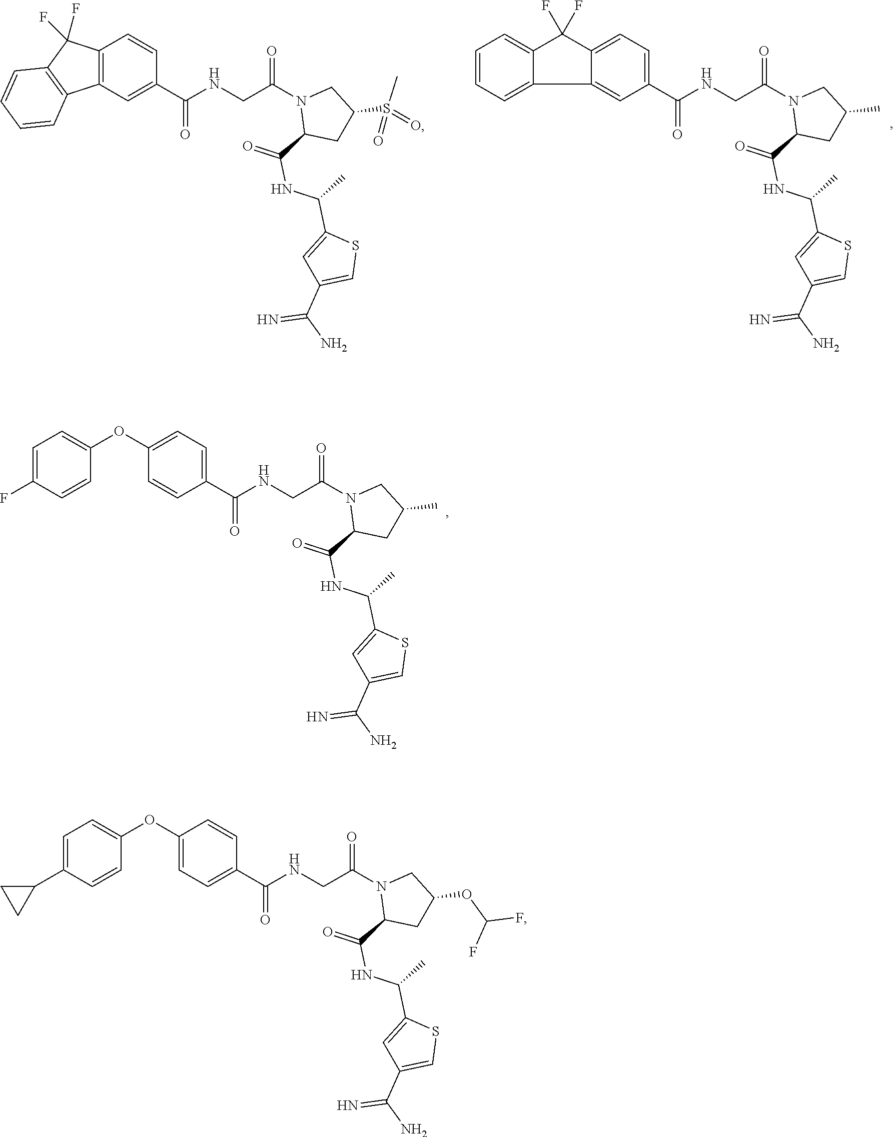 [SMILES: C[C@@H](NC(=O)[C@@H]1C[C@@H](S(C)(=O)=O)CN1C(=O)CNC(=O)c1ccc2c(c1)-c1ccccc1C2(F)F)c1cc(C(=N)N)cs1 ; C[C@@H]1C[C@@H](C(=O)N[C@H](C)c2cc(C(=N)N)cs2)N(C(=O)CNC(=O)c2ccc3c(c2)-c2ccccc2C3(F)F)C1 ; C[C@@H]1C[C@@H](C(=O)N[C@H](C)c2cc(C(=N)N)cs2)N(C(=O)CNC(=O)c2ccc(Oc3ccc(F)cc3)cc2)C1 ; C[C@@H](NC(=O)[C@@H]1C[C@@H](OC(F)F)CN1C(=O)CNC(=O)c1ccc(Oc2ccc(C3CC3)cc2)cc1)c1cc(C(=N)N)cs1] 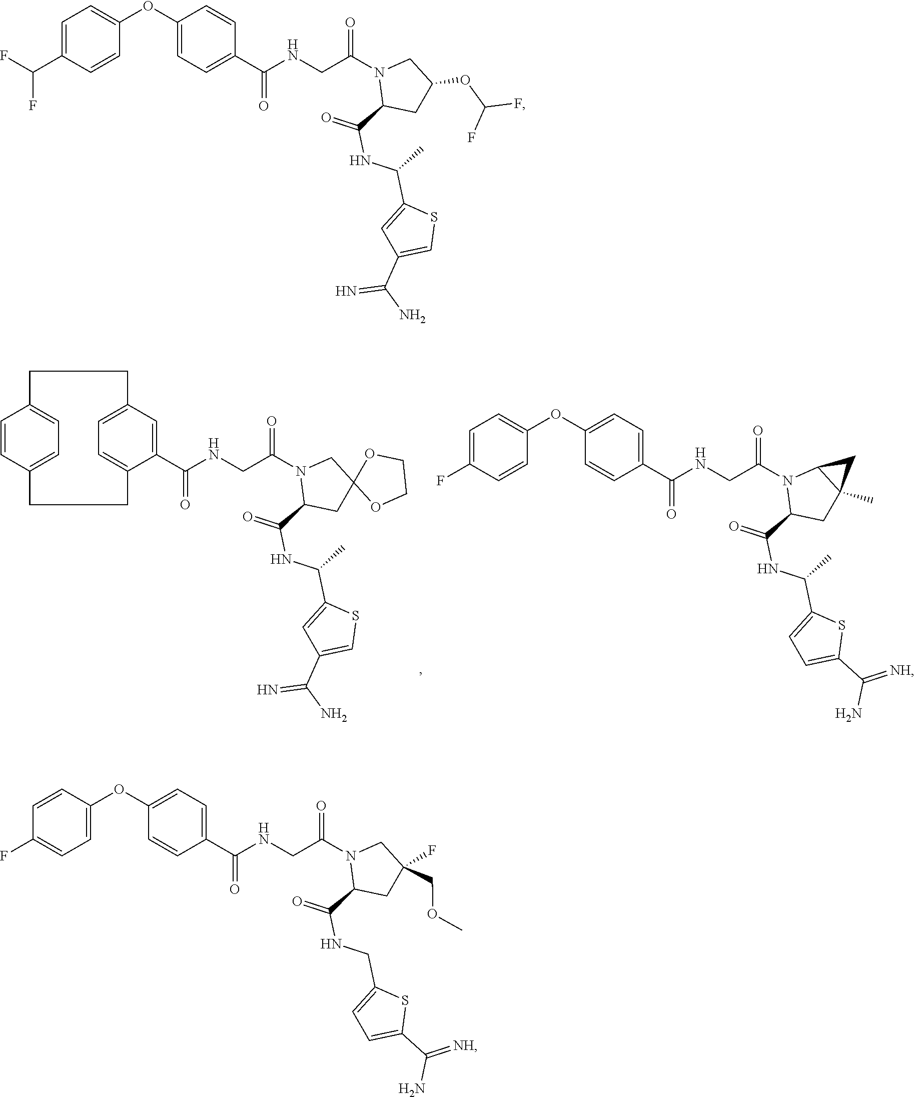 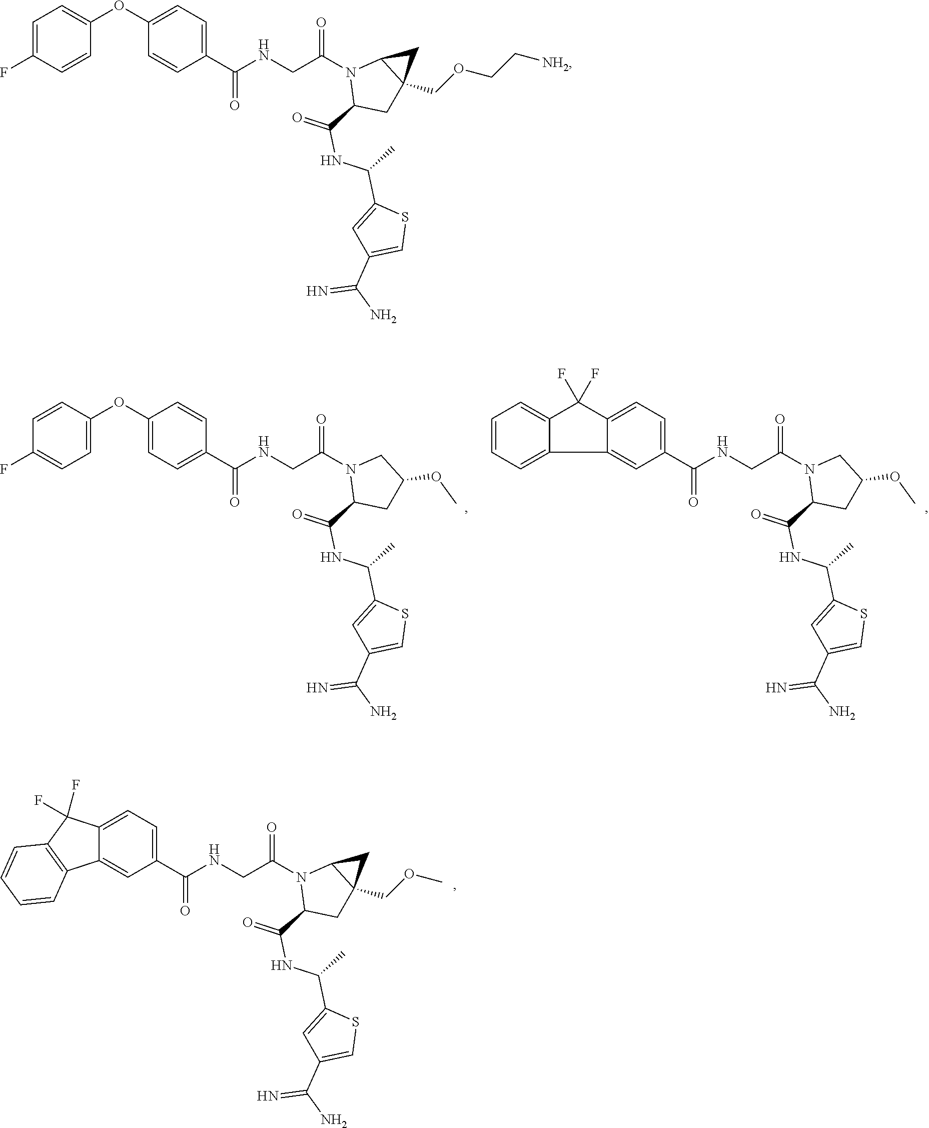 [SMILES: C[C@@H](NC(=O)[C@@H]1C[C@]2(COCCN)C[C@@H]2N1C(=O)CNC(=O)c1ccc(Oc2ccc(F)cc2)cc1)c1cc(C(=N)N)cs1 ; CO[C@@H]1C[C@@H](C(=O)N[C@H](C)c2cc(C(=N)N)cs2)N(C(=O)CNC(=O)c2ccc(Oc3ccc(F)cc3)cc2)C1 ; CO[C@@H]1C[C@@H](C(=O)N[C@H](C)c2cc(C(=N)N)cs2)N(C(=O)CNC(=O)c2ccc3c(c2)-c2ccccc2C3(F)F)C1 ; COC[C@@]12C[C@@H]1N(C(=O)CNC(=O)c1ccc3c(c1)-c1ccccc1C3(F)F)[C@H](C(=O)N[C@H](C)c1cc(C(=N)N)cs1)C2] 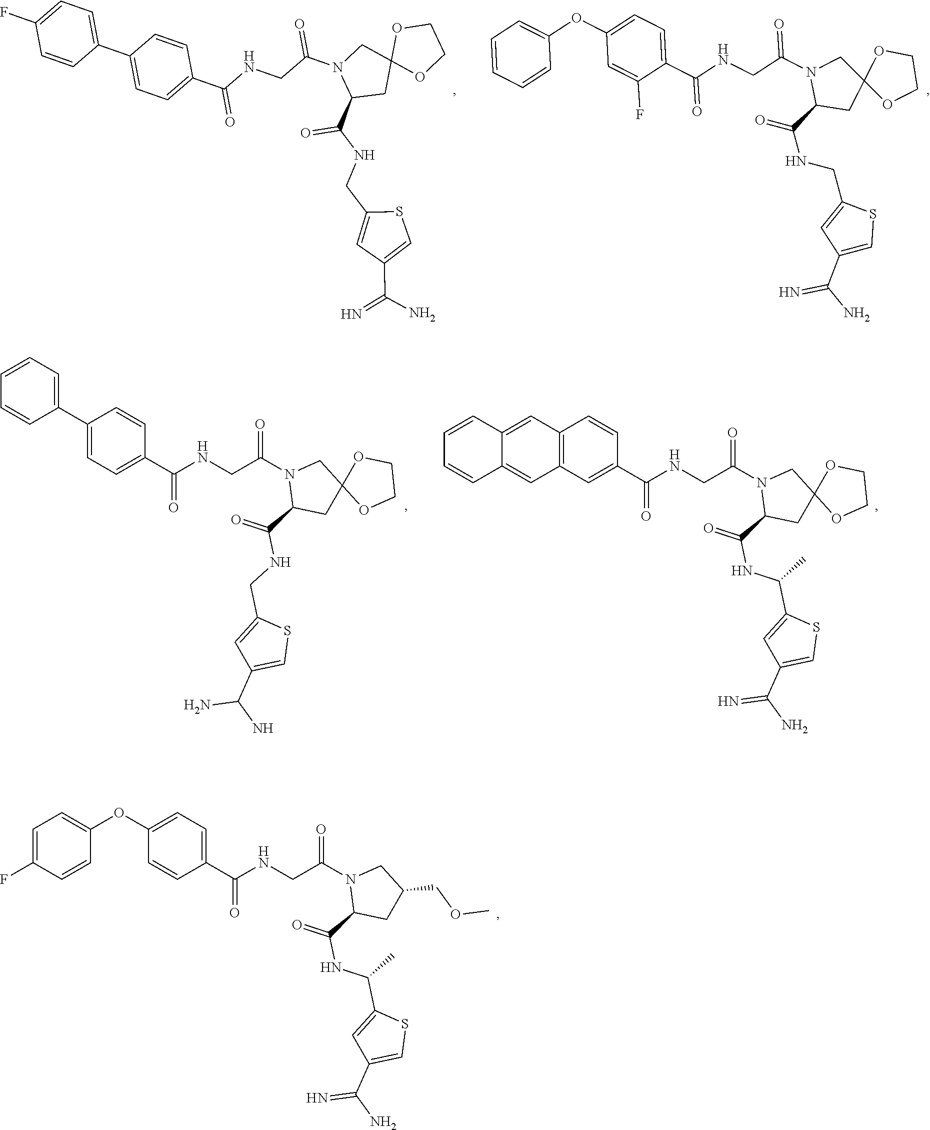 [SMILES: N=C(N)c1csc(CNC(=O)[C@@H]2CC3(CN2C(=O)CNC(=O)c2ccc(-c4ccc(F)cc4)cc2)OCCO3)c1 ; N=C(N)c1csc(CNC(=O)[C@@H]2CC3(CN2C(=O)CNC(=O)c2ccc(Oc4ccccc4)cc2F)OCCO3)c1 ; NC(N)c1csc(CNC(=O)[C@@H]2CC3(CN2C(=O)CNC(=O)c2ccc(-c4ccccc4)cc2)OCCO3)c1 ; C[C@@H](NC(=O)[C@@H]1CC2(CN1C(=O)CNC(=O)c1ccc3cc4ccccc4cc3c1)OCCO2)c1cc(C(=N)N)cs1 ; COC[C@@H]1C[C@@H](C(=O)N[C@H](C)c2cc(C(=N)N)cs2)N(C(=O)CNC(=O)c2ccc(Oc3ccc(F)cc3)cc2)C1] 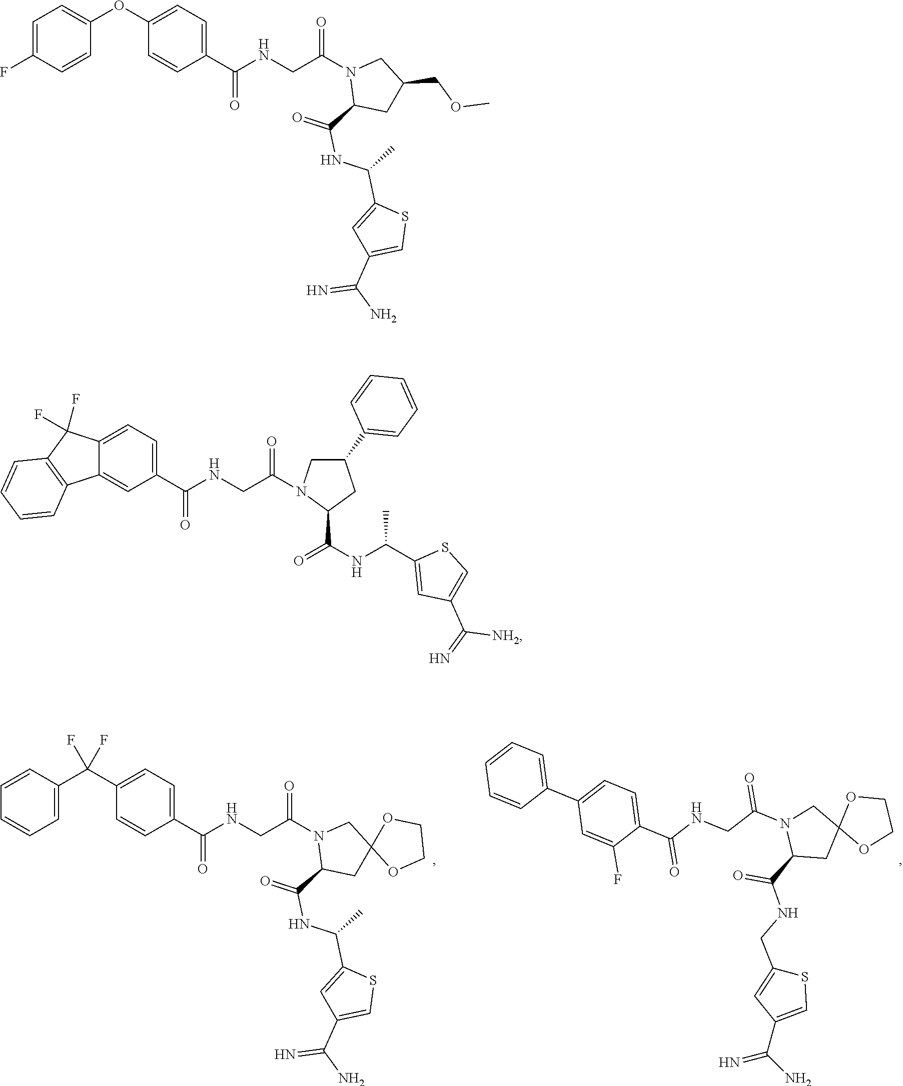 [SMILES: COC[C@H]1C[C@@H](C(=O)N[C@H](C)c2cc(C(=N)N)cs2)N(C(=O)CNC(=O)c2ccc(Oc3ccc(F)cc3)cc2)C1 ; C[C@@H](NC(=O)[C@@H]1C[C@@H](c2ccccc2)CN1C(=O)CNC(=O)c1ccc2c(c1)-c1ccccc1C2(F)F)c1cc(C(=N)N)cs1 ; C[C@@H](NC(=O)[C@@H]1CC2(CN1C(=O)CNC(=O)c1ccc(C(F)(F)c3ccccc3)cc1)OCCO2)c1cc(C(=N)N)cs1 ; N=C(N)c1csc(CNC(=O)[C@@H]2CC3(CN2C(=O)CNC(=O)c2ccc(-c4ccccc4)cc2F)OCCO3)c1] 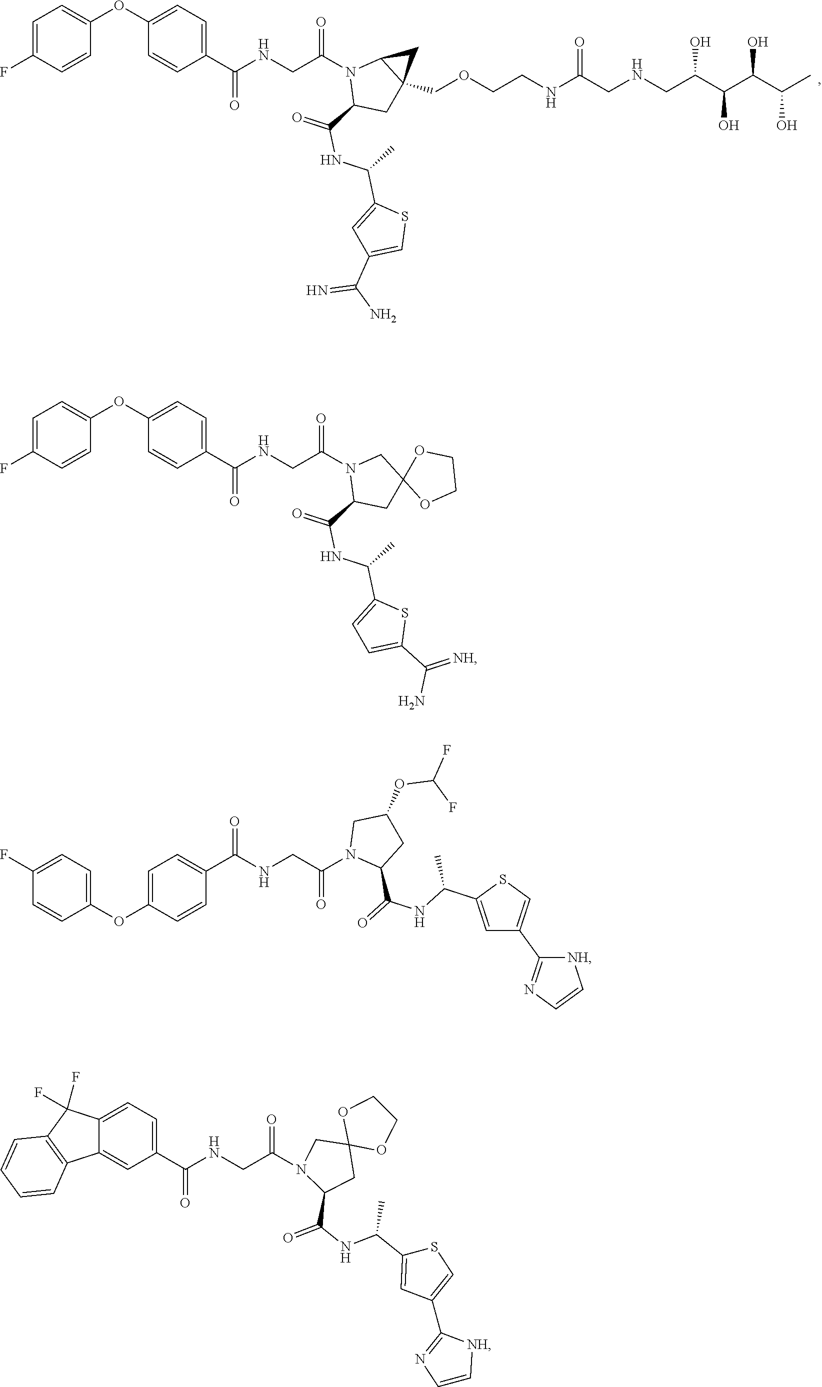 [SMILES: C[C@H](O)[C@H](O)[C@@H](O)[C@@H](O)CNCC(=O)NCCOC[C@@]12C[C@@H]1N(C(=O)CNC(=O)c1ccc(Oc3ccc(F)cc3)cc1)[C@H](C(=O)N[C@H](C)c1cc(C(=N)N)cs1)C2 ; C[C@@H](NC(=O)[C@@H]1CC2(CN1C(=O)CNC(=O)c1ccc(Oc3ccc(F)cc3)cc1)OCCO2)c1ccc(C(=N)N)s1 ; C[C@@H](NC(=O)[C@@H]1C[C@@H](OC(F)F)CN1C(=O)CNC(=O)c1ccc(Oc2ccc(F)cc2)cc1)c1cc(-c2ncc[nH]2)cs1 ; C[C@@H](NC(=O)[C@@H]1CC2(CN1C(=O)CNC(=O)c1ccc3c(c1)-c1ccccc1C3(F)F)OCCO2)c1cc(-c2ncc[nH]2)cs1] 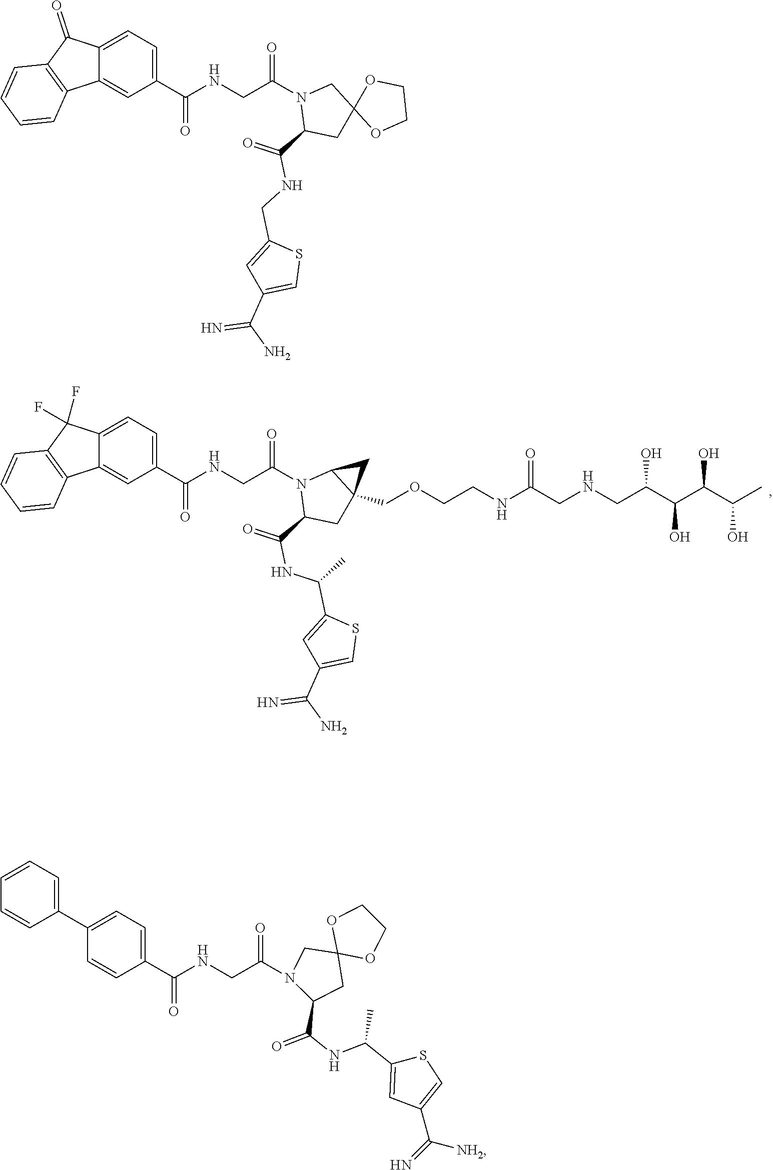 [SMILES: N=C(N)c1csc(CNC(=O)[C@@H]2CC3(CN2C(=O)CNC(=O)c2ccc4c(c2)-c2ccccc2C4=O)OCCO3)c1 ; C[C@H](O)[C@H](O)[C@@H](O)[C@@H](O)CNCC(=O)NCCOC[C@@]12C[C@@H]1N(C(=O)CNC(=O)c1ccc3c(c1)-c1ccccc1C3(F)F)[C@H](C(=O)N[C@H](C)c1cc(C(=N)N)cs1)C2 ; C[C@@H](NC(=O)[C@@H]1CC2(CN1C(=O)CNC(=O)c1ccc(-c3ccccc3)cc1)OCCO2)c1cc(C(=N)N)cs1] 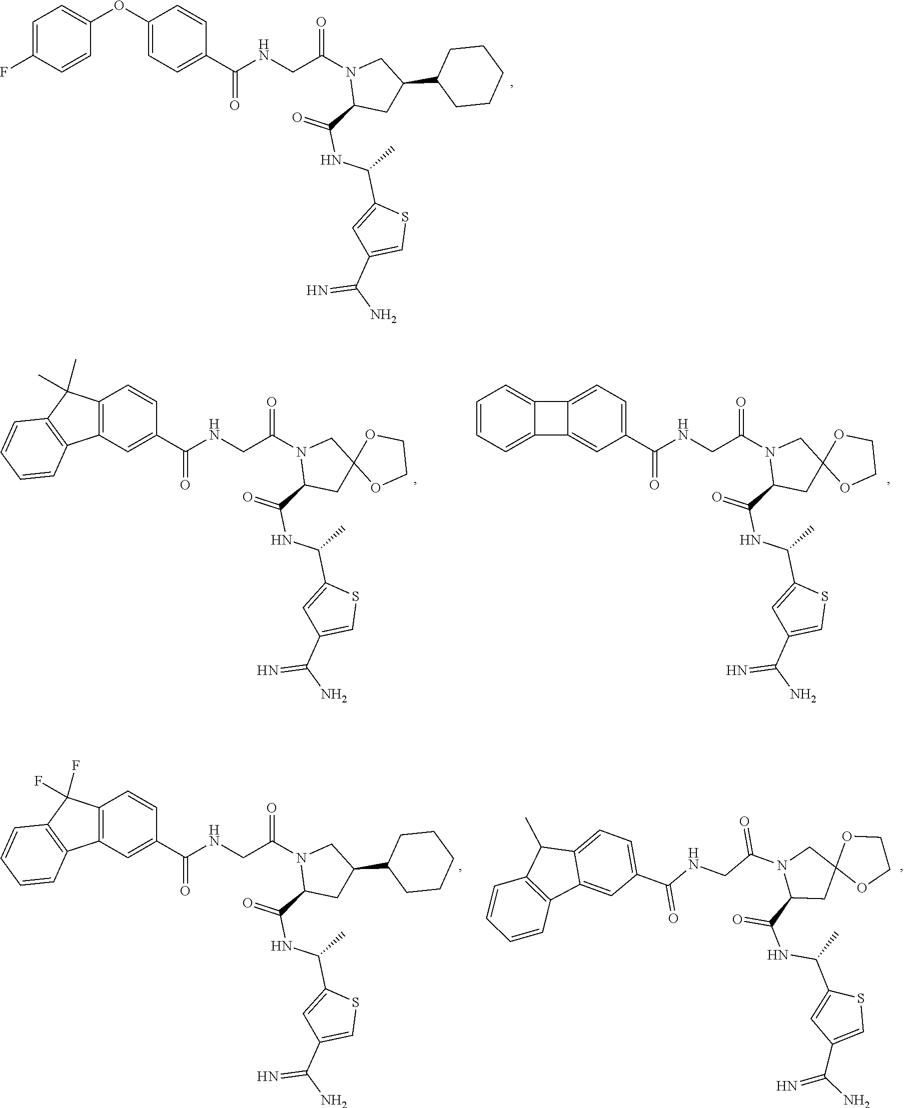 [SMILES: C[C@@H](NC(=O)[C@@H]1C[C@H](C2CCCCC2)CN1C(=O)CNC(=O)c1ccc(Oc2ccc(F)cc2)cc1)c1cc(C(=N)N)cs1 ; C[C@@H](NC(=O)[C@@H]1CC2(CN1C(=O)CNC(=O)c1ccc3c(c1)-c1ccccc1C3(C)C)OCCO2)c1cc(C(=N)N)cs1 ; C[C@@H](NC(=O)[C@@H]1CC2(CN1C(=O)CNC(=O)c1ccc3c(c1)-c1ccccc1-3)OCCO2)c1cc(C(=N)N)cs1 ; C[C@@H](NC(=O)[C@@H]1C[C@H](C2CCCCC2)CN1C(=O)CNC(=O)c1ccc2c(c1)-c1ccccc1C2(F)F)c1cc(C(=N)N)cs1 ; CC1c2ccccc2-c2cc(C(=O)NCC(=O)N3CC4(C[C@H]3C(=O)N[C@H](C)c3cc(C(=N)N)cs3)OCCO4)ccc21] 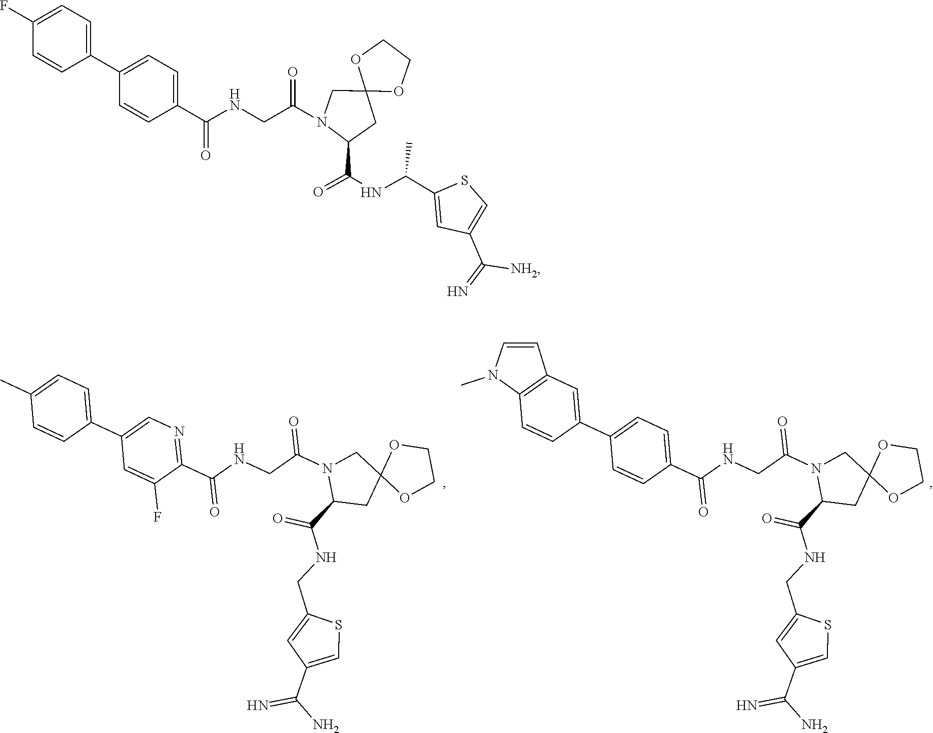 [SMILES: C[C@@H](NC(=O)[C@@H]1CC2(CN1C(=O)CNC(=O)c1ccc(-c3ccc(F)cc3)cc1)OCCO2)c1cc(C(=N)N)cs1 ; Cc1ccc(-c2cnc(C(=O)NCC(=O)N3CC4(C[C@H]3C(=O)NCc3cc(C(=N)N)cs3)OCCO4)c(F)c2)cc1 ; Cn1ccc2cc(-c3ccc(C(=O)NCC(=O)N4CC5(C[C@H]4C(=O)NCc4cc(C(=N)N)cs4)OCCO5)cc3)ccc21] 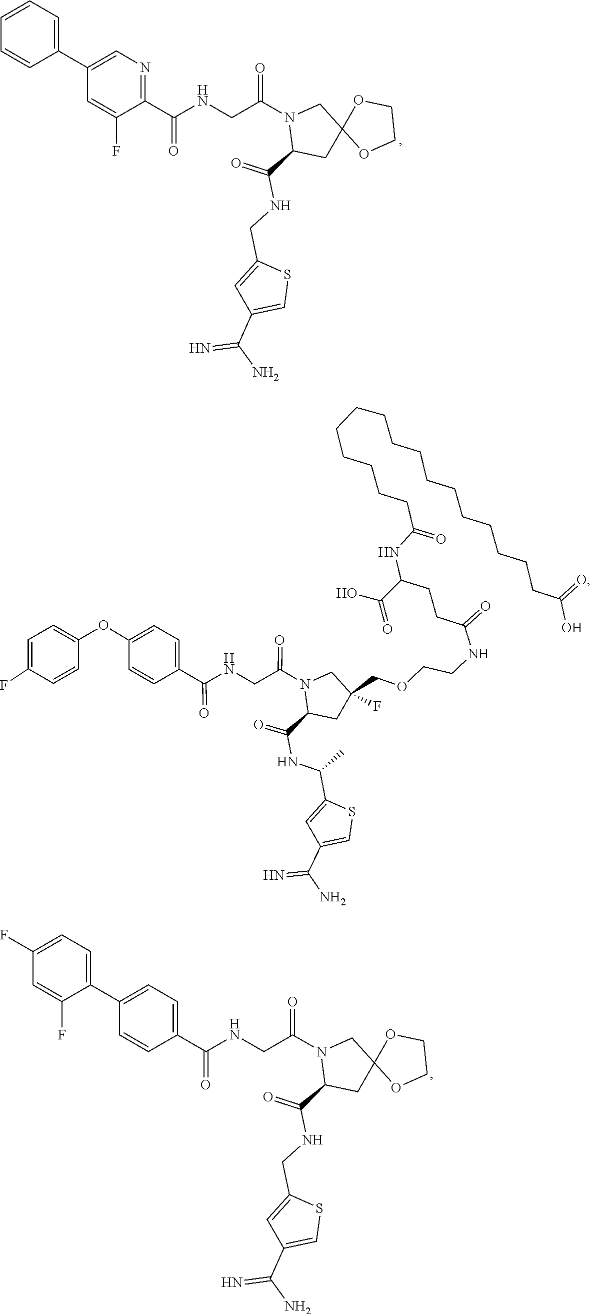 [SMILES: N=C(N)c1csc(CNC(=O)[C@@H]2CC3(CN2C(=O)CNC(=O)c2ncc(-c4ccccc4)cc2F)OCCO3)c1 ; C[C@@H](NC(=O)[C@@H]1C[C@](F)(COCCNC(=O)CCC(NC(=O)CCCCCCCCCCCCCCCCC(=O)O)C(=O)O)CN1C(=O)CNC(=O)c1ccc(Oc2ccc(F)cc2)cc1)c1cc(C(=N)N)cs1 ; N=C(N)c1csc(CNC(=O)[C@@H]2CC3(CN2C(=O)CNC(=O)c2ccc(-c4ccc(F)cc4F)cc2)OCCO3)c1] 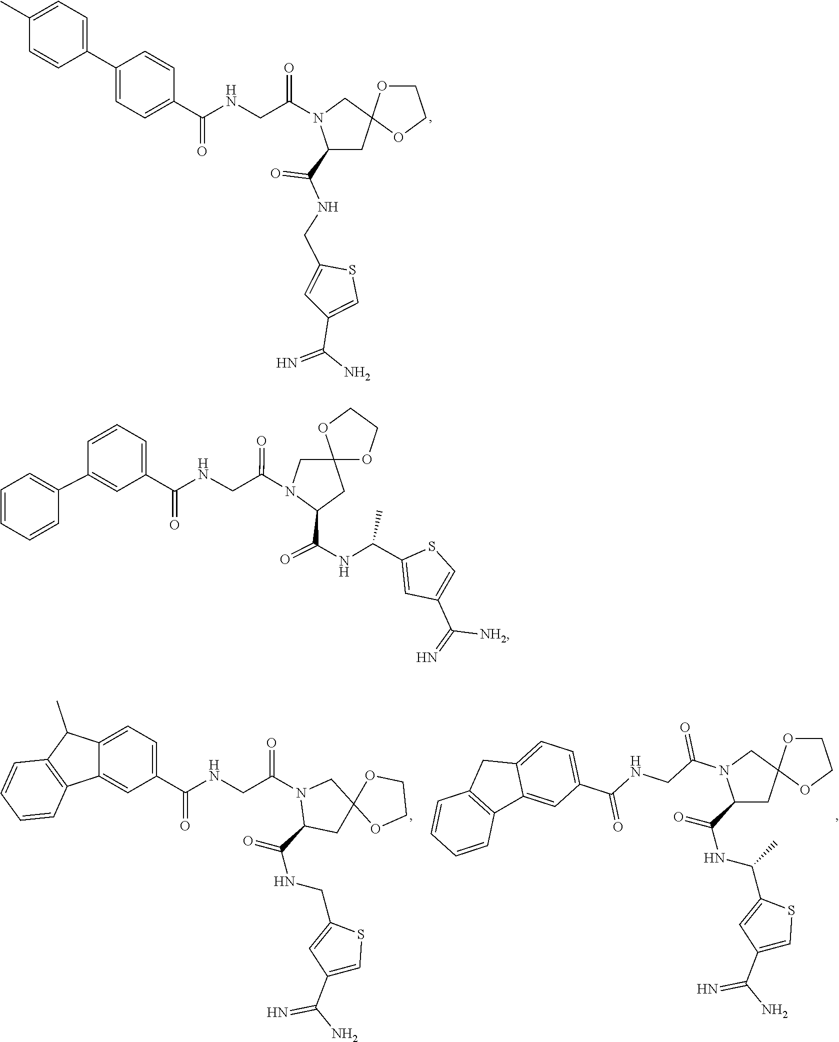 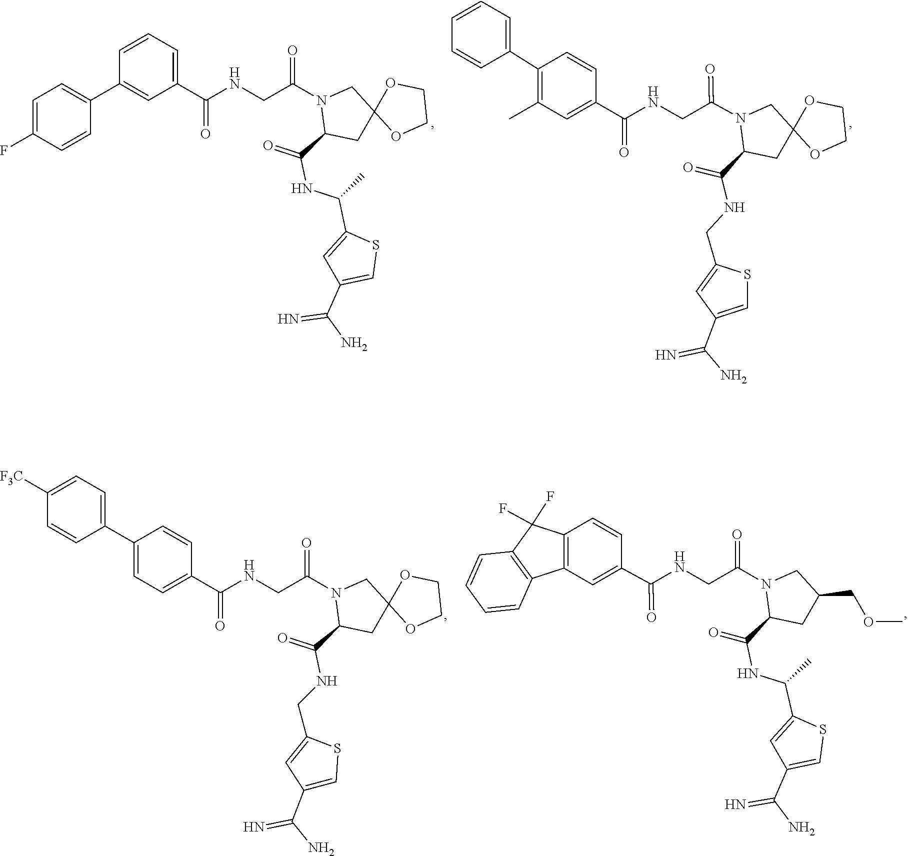 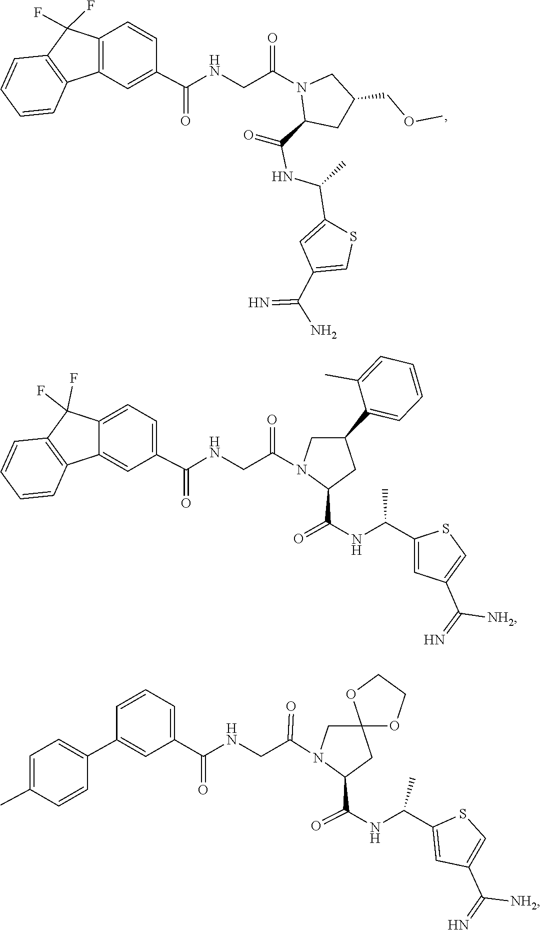 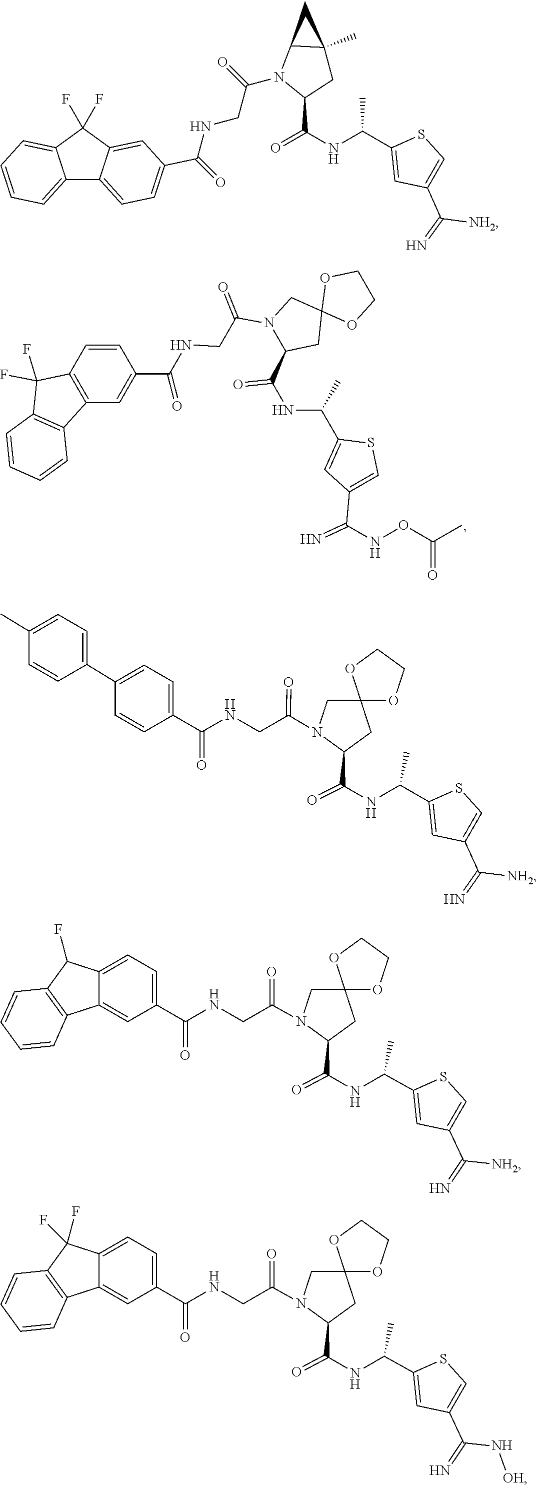 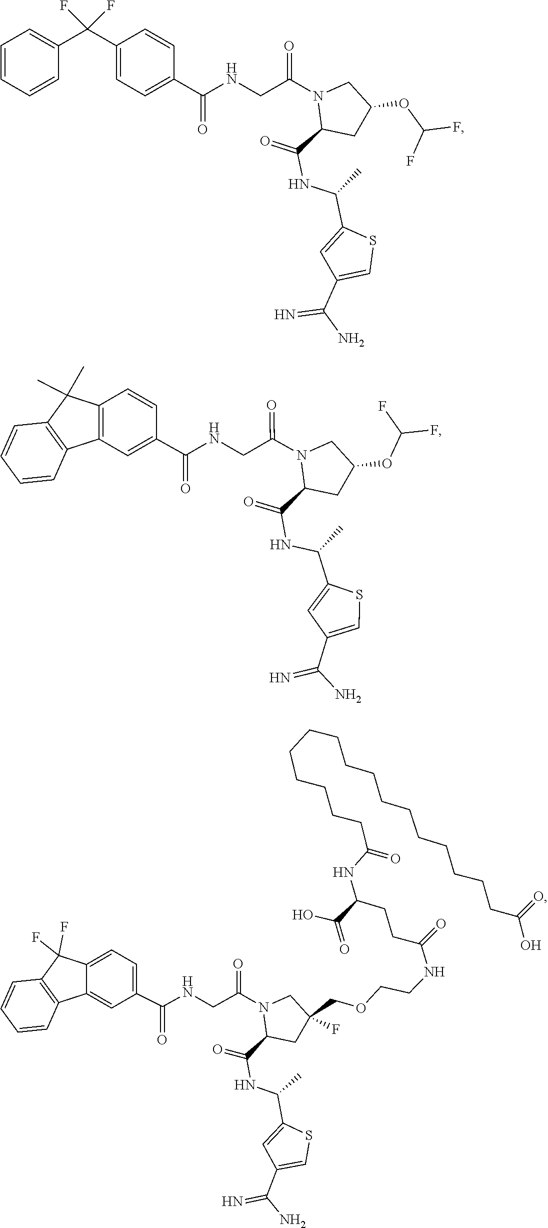 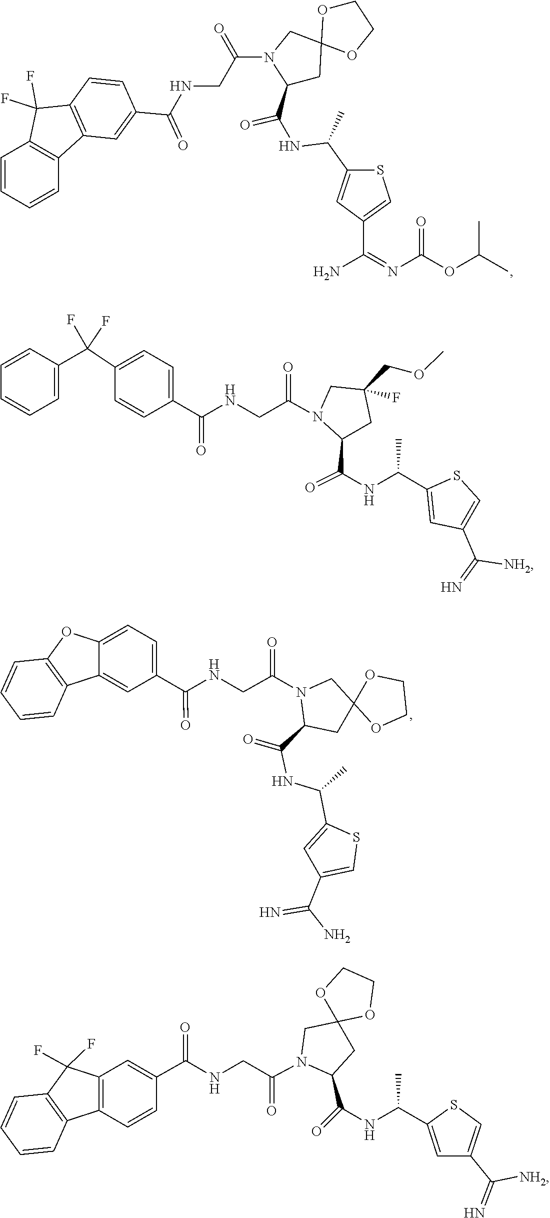 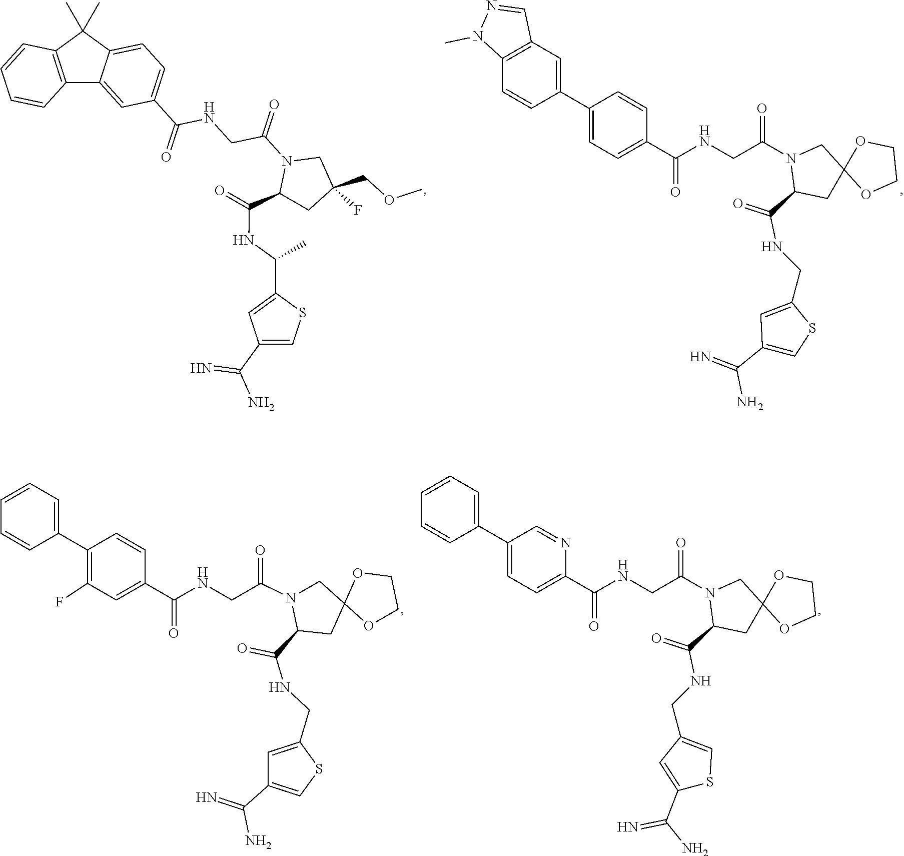 [SMILES: COC[C@@]1(F)C[C@@H](C(=O)N[C@H](C)c2cc(C(=N)N)cs2)N(C(=O)CNC(=O)c2ccc3c(c2)-c2ccccc2C3(C)C)C1 ; Cn1ncc2cc(-c3ccc(C(=O)NCC(=O)N4CC5(C[C@H]4C(=O)NCc4cc(C(=N)N)cs4)OCCO5)cc3)ccc21 ; N=C(N)c1csc(CNC(=O)[C@@H]2CC3(CN2C(=O)CNC(=O)c2ccc(-c4ccccc4)c(F)c2)OCCO3)c1 ; N=C(N)c1cc(CNC(=O)[C@@H]2CC3(CN2C(=O)CNC(=O)c2ccc(-c4ccccc4)cn2)OCCO3)cs1] 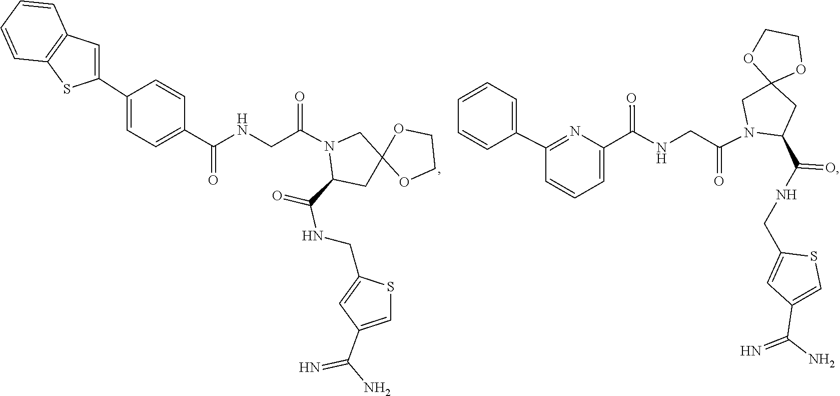 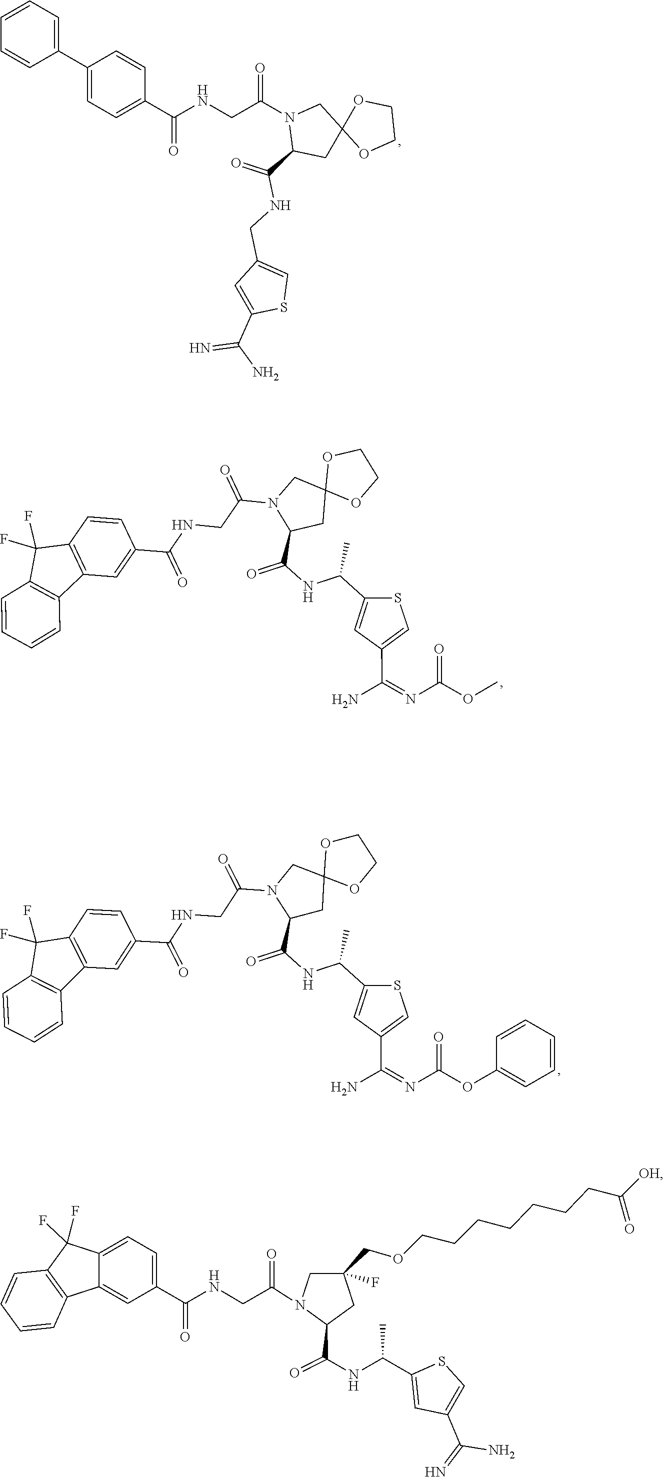 [SMILES: N=C(N)c1cc(CNC(=O)[C@@H]2CC3(CN2C(=O)CNC(=O)c2ccc(-c4ccccc4)cc2)OCCO3)cs1 ; COC(=O)/N=C(/N)c1csc([C@@H](C)NC(=O)[C@@H]2CC3(CN2C(=O)CNC(=O)c2ccc4c(c2)-c2ccccc2C4(F)F)OCCO3)c1 ; C[C@@H](NC(=O)[C@@H]1CC2(CN1C(=O)CNC(=O)c1ccc3c(c1)-c1ccccc1C3(F)F)OCCO2)c1cc(/C(N)=N\C(=O)Oc2ccccc2)cs1 ; C[C@@H](NC(=O)[C@@H]1C[C@](F)(COCCCCCCCC(=O)O)CN1C(=O)CNC(=O)c1ccc2c(c1)-c1ccccc1C2(F)F)c1cc(C(=N)N)cs1] 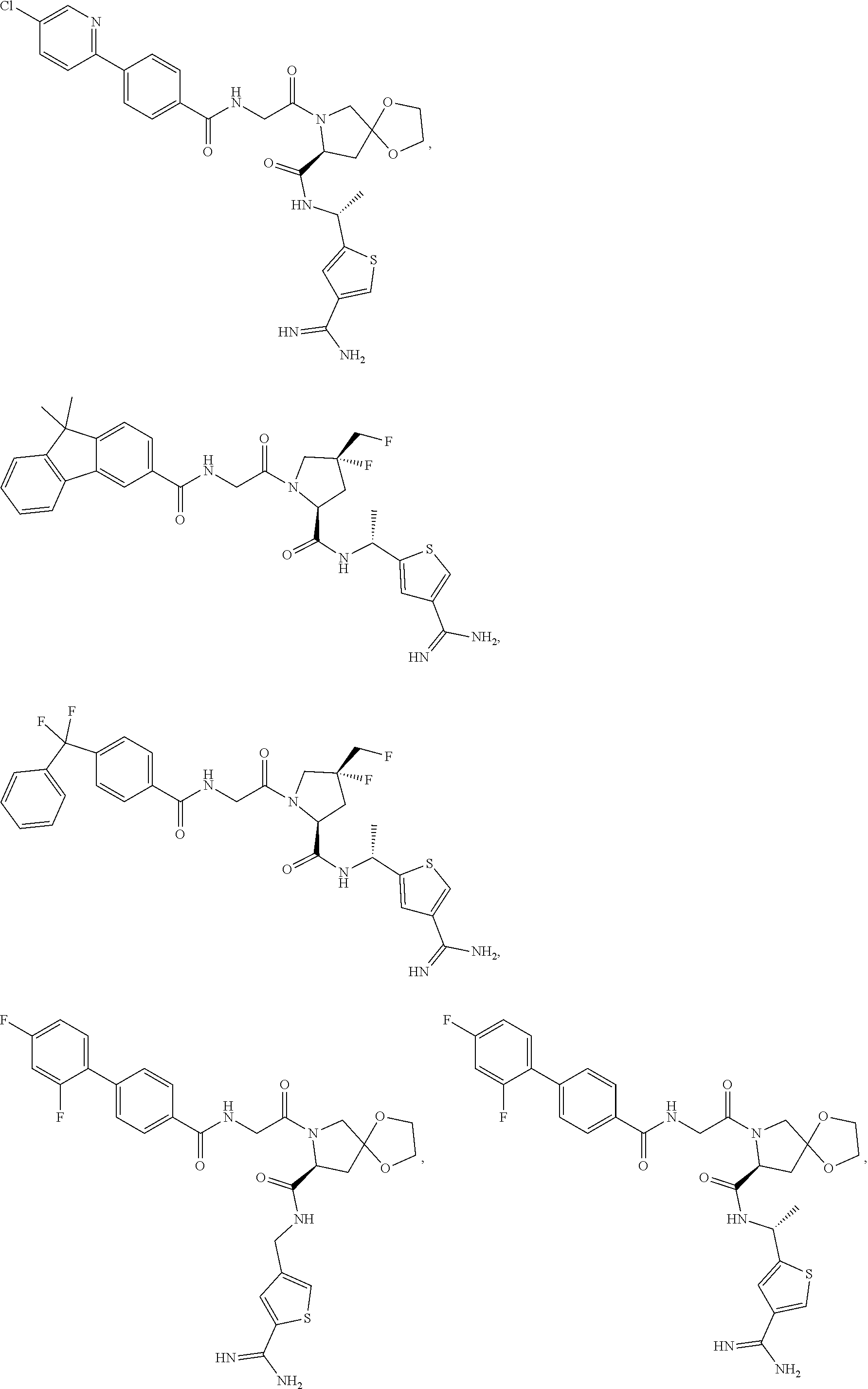 [SMILES: C[C@@H](NC(=O)[C@@H]1CC2(CN1C(=O)CNC(=O)c1ccc(-c3ccc(Cl)cn3)cc1)OCCO2)c1cc(C(=N)N)cs1 ; C[C@@H](NC(=O)[C@@H]1C[C@](F)(CF)CN1C(=O)CNC(=O)c1ccc2c(c1)-c1ccccc1C2(C)C)c1cc(C(=N)N)cs1 ; C[C@@H](NC(=O)[C@@H]1C[C@](F)(CF)CN1C(=O)CNC(=O)c1ccc(C(F)(F)c2ccccc2)cc1)c1cc(C(=N)N)cs1 ; N=C(N)c1cc(CNC(=O)[C@@H]2CC3(CN2C(=O)CNC(=O)c2ccc(-c4ccc(F)cc4F)cc2)OCCO3)cs1 ; C[C@@H](NC(=O)[C@@H]1CC2(CN1C(=O)CNC(=O)c1ccc(-c3ccc(F)cc3F)cc1)OCCO2)c1cc(C(=N)N)cs1] 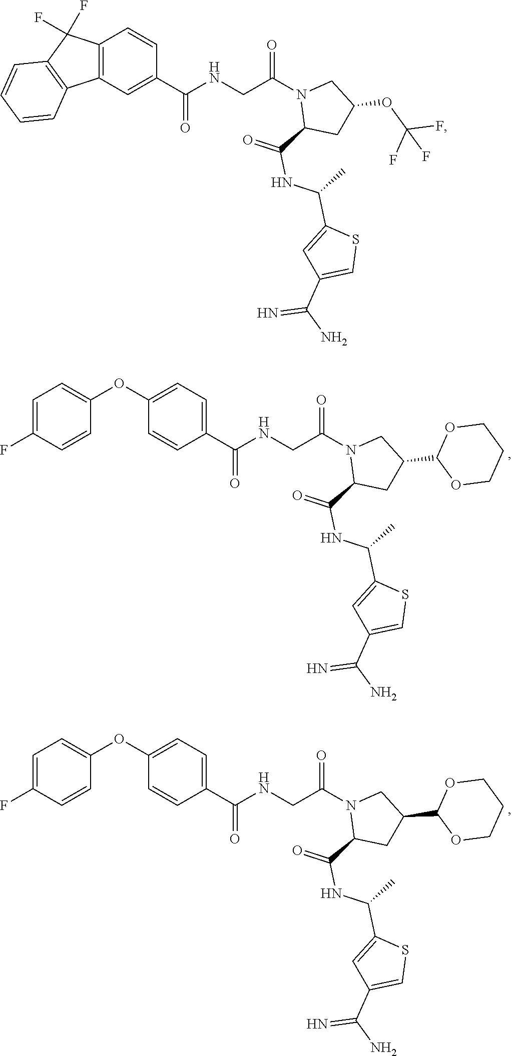 [SMILES: C[C@@H](NC(=O)[C@@H]1C[C@@H](OC(F)(F)F)CN1C(=O)CNC(=O)c1ccc2c(c1)-c1ccccc1C2(F)F)c1cc(C(=N)N)cs1 ; C[C@@H](NC(=O)[C@@H]1C[C@@H](C2OCCCO2)CN1C(=O)CNC(=O)c1ccc(Oc2ccc(F)cc2)cc1)c1cc(C(=N)N)cs1 ; C[C@@H](NC(=O)[C@@H]1C[C@H](C2OCCCO2)CN1C(=O)CNC(=O)c1ccc(Oc2ccc(F)cc2)cc1)c1cc(C(=N)N)cs1] 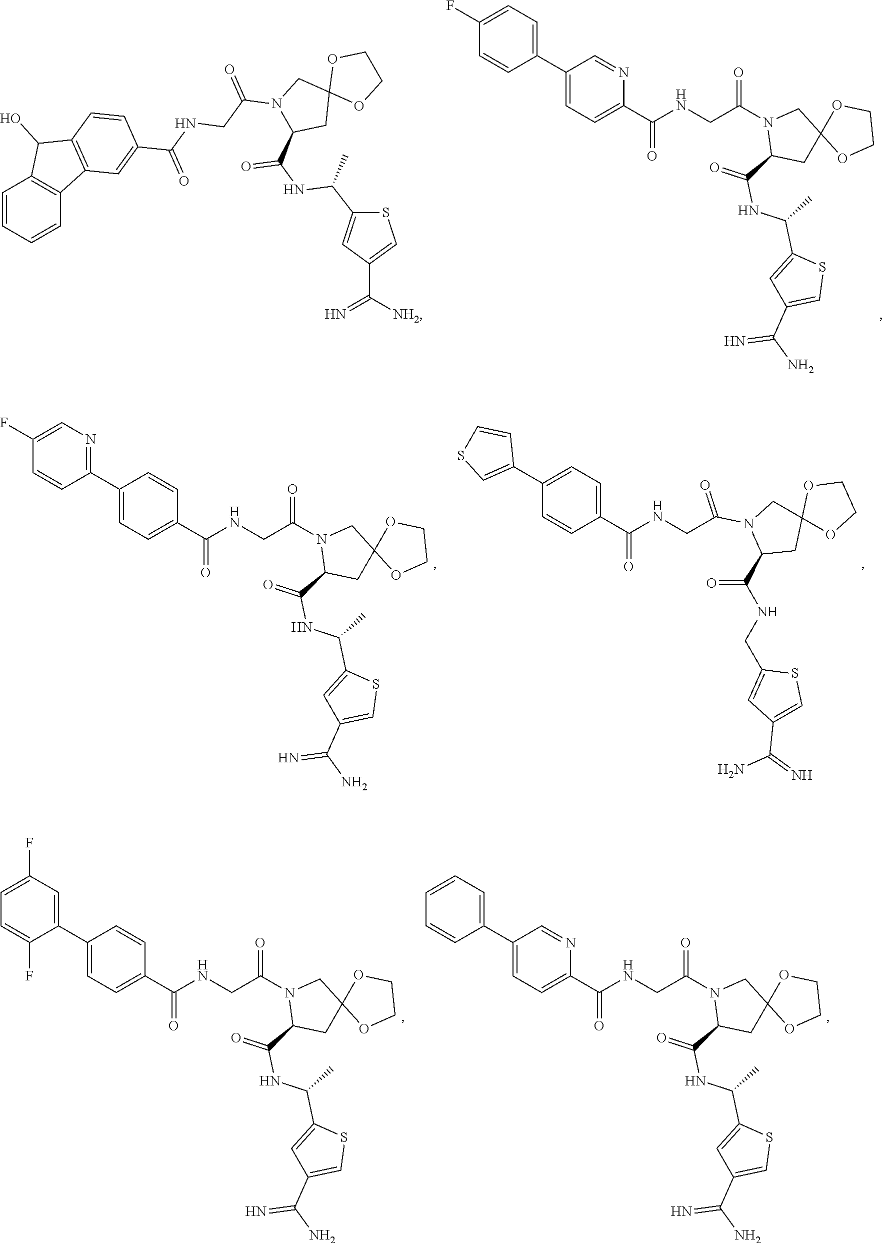 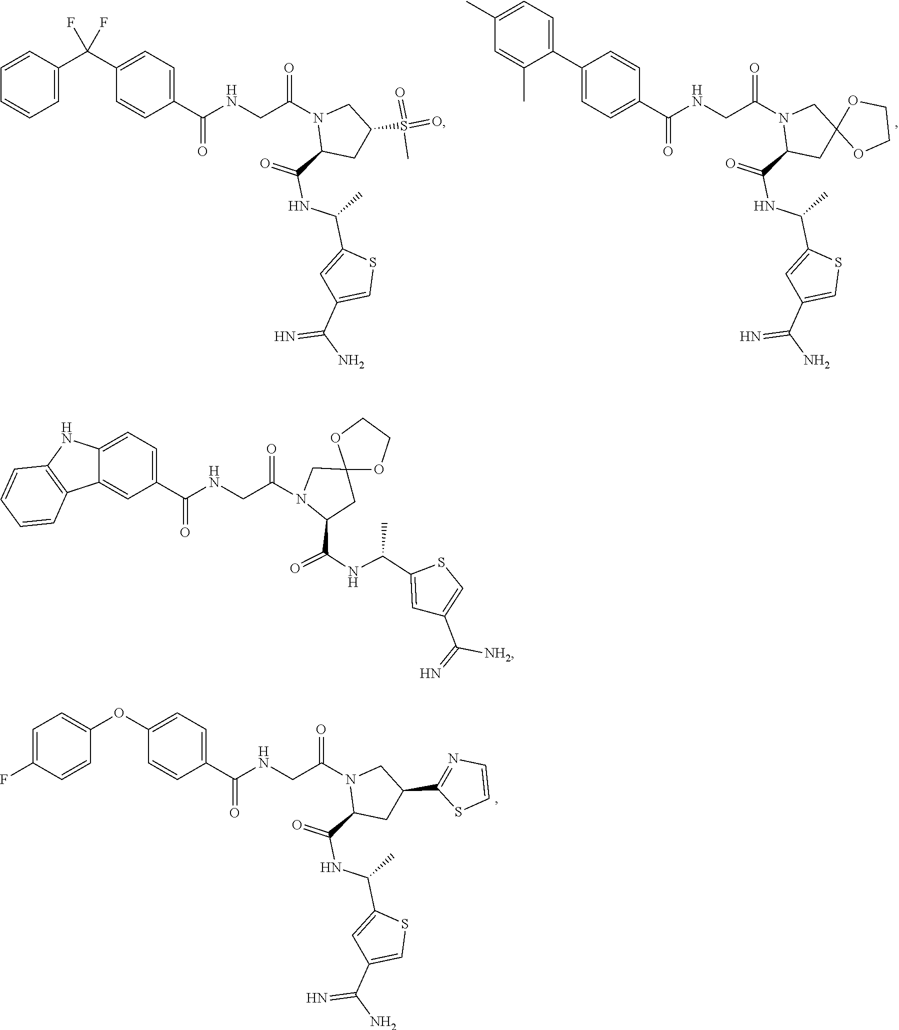 [SMILES: C[C@@H](NC(=O)[C@@H]1C[C@@H](S(C)(=O)=O)CN1C(=O)CNC(=O)c1ccc(C(F)(F)c2ccccc2)cc1)c1cc(C(=N)N)cs1 ; Cc1ccc(-c2ccc(C(=O)NCC(=O)N3CC4(C[C@H]3C(=O)N[C@H](C)c3cc(C(=N)N)cs3)OCCO4)cc2)c(C)c1 ; C[C@@H](NC(=O)[C@@H]1CC2(CN1C(=O)CNC(=O)c1ccc3[nH]c4ccccc4c3c1)OCCO2)c1cc(C(=N)N)cs1 ; C[C@@H](NC(=O)[C@@H]1C[C@H](c2nccs2)CN1C(=O)CNC(=O)c1ccc(Oc2ccc(F)cc2)cc1)c1cc(C(=N)N)cs1] 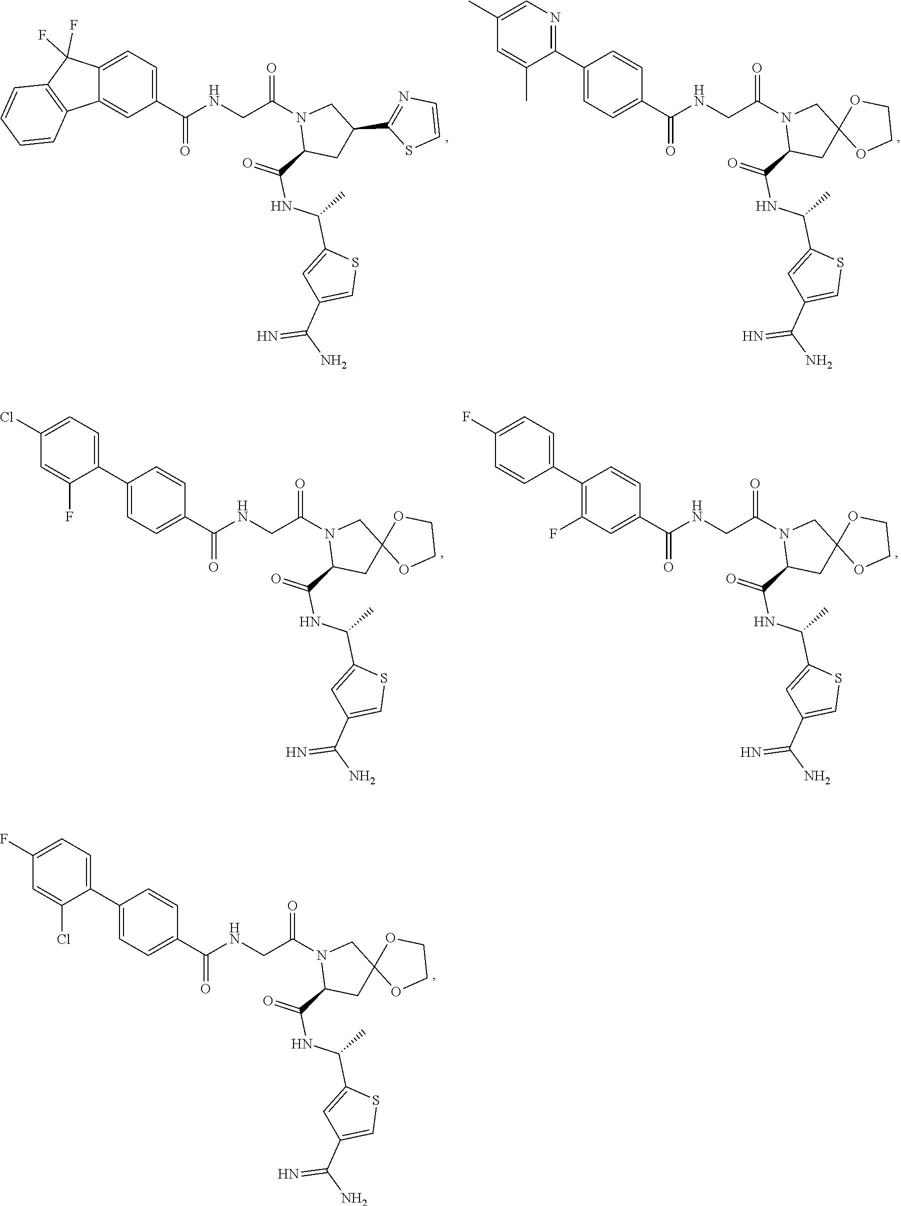 [SMILES: C[C@@H](NC(=O)[C@@H]1C[C@H](c2nccs2)CN1C(=O)CNC(=O)c1ccc2c(c1)-c1ccccc1C2(F)F)c1cc(C(=N)N)cs1 ; Cc1cnc(-c2ccc(C(=O)NCC(=O)N3CC4(C[C@H]3C(=O)N[C@H](C)c3cc(C(=N)N)cs3)OCCO4)cc2)c(C)c1 ; C[C@@H](NC(=O)[C@@H]1CC2(CN1C(=O)CNC(=O)c1ccc(-c3ccc(Cl)cc3F)cc1)OCCO2)c1cc(C(=N)N)cs1 ; C[C@@H](NC(=O)[C@@H]1CC2(CN1C(=O)CNC(=O)c1ccc(-c3ccc(F)cc3)c(F)c1)OCCO2)c1cc(C(=N)N)cs1 ; C[C@@H](NC(=O)[C@@H]1CC2(CN1C(=O)CNC(=O)c1ccc(-c3ccc(F)cc3Cl)cc1)OCCO2)c1cc(C(=N)N)cs1] 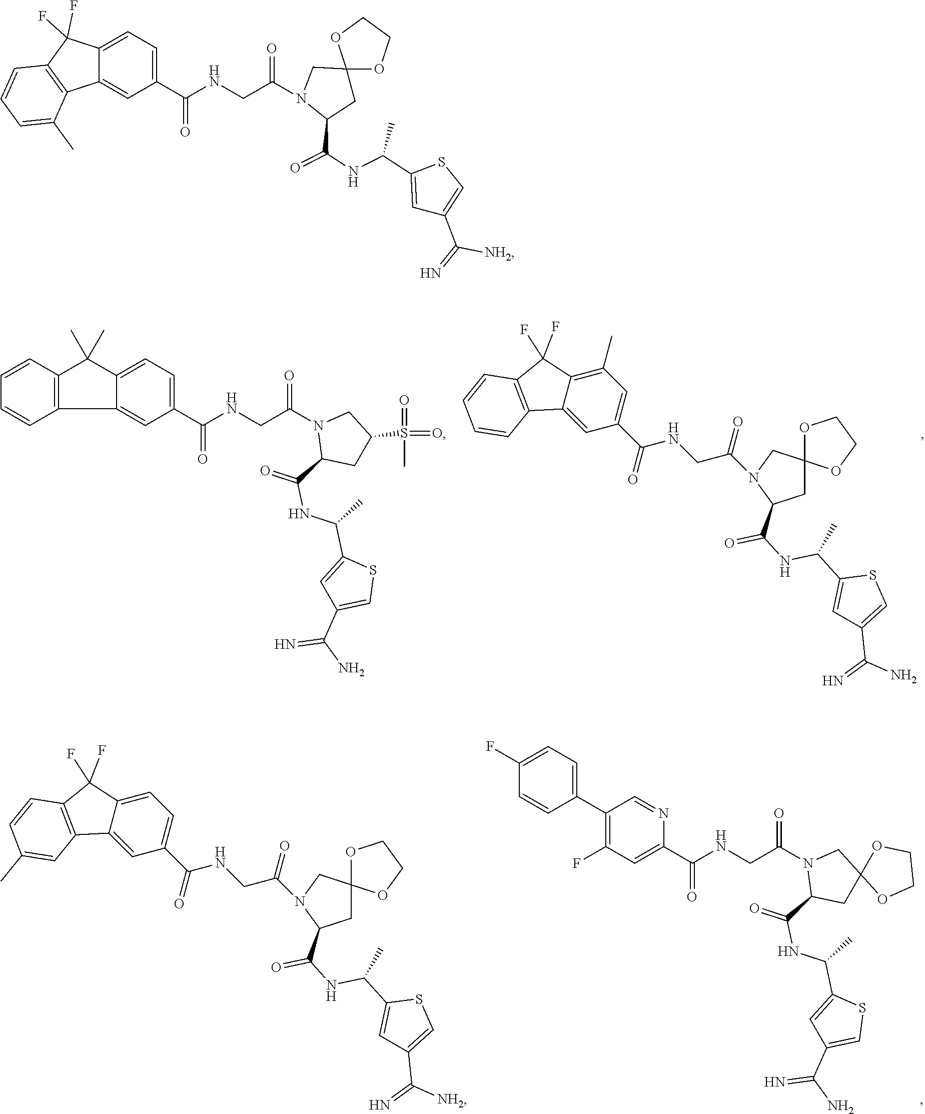 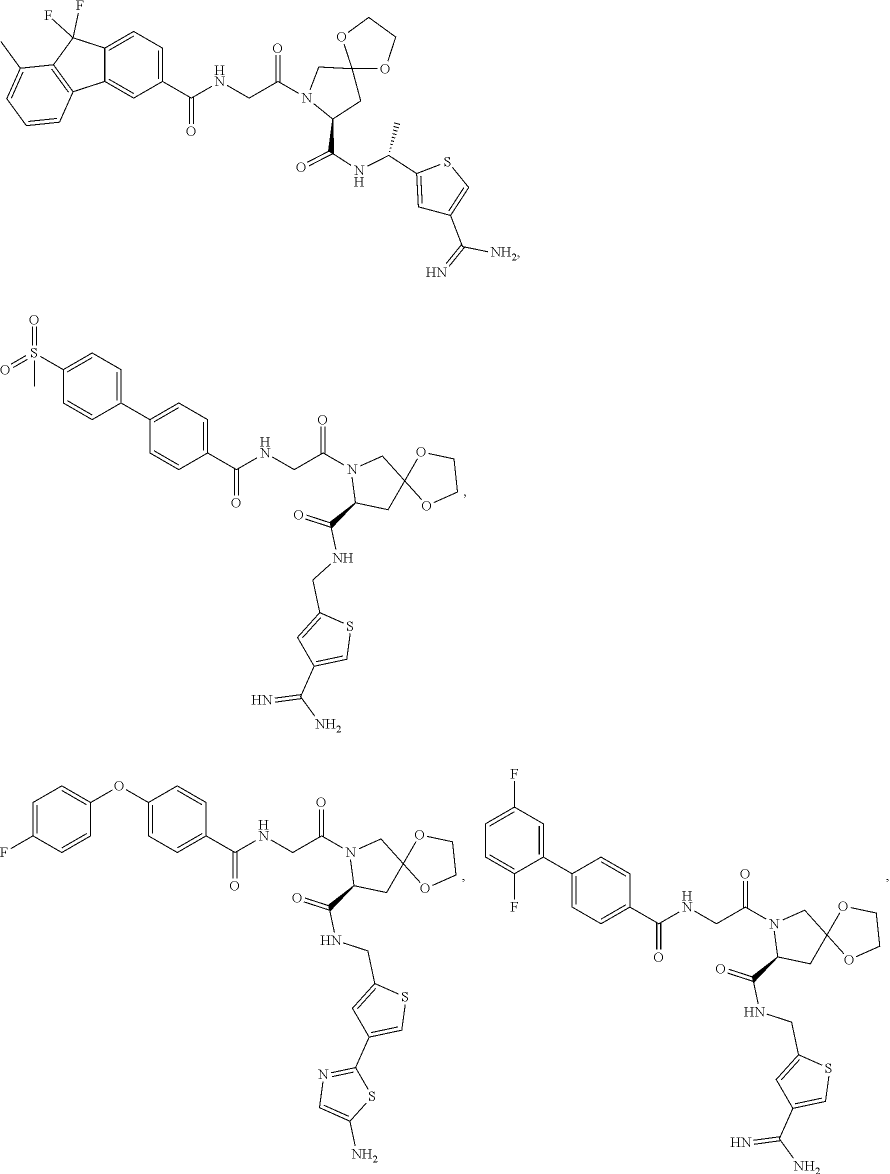 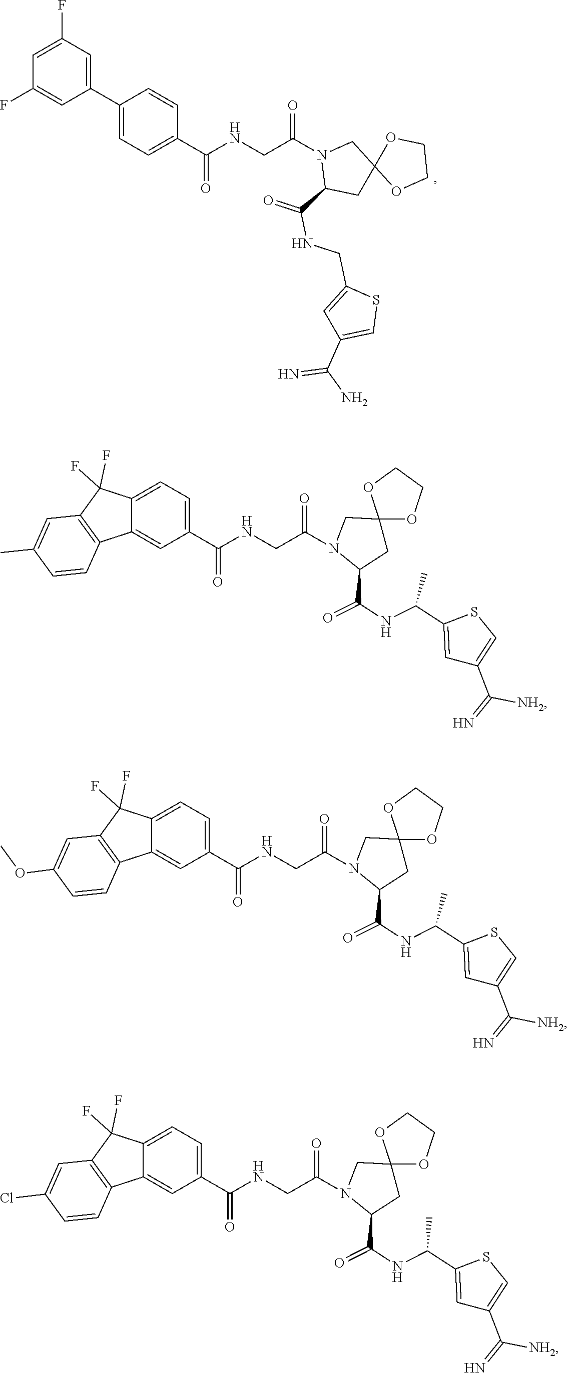 [SMILES: N=C(N)c1csc(CNC(=O)[C@@H]2CC3(CN2C(=O)CNC(=O)c2ccc(-c4cc(F)cc(F)c4)cc2)OCCO3)c1 ; Cc1ccc2c(c1)C(F)(F)c1ccc(C(=O)NCC(=O)N3CC4(C[C@H]3C(=O)N[C@H](C)c3cc(C(=N)N)cs3)OCCO4)cc1-2 ; COc1ccc2c(c1)C(F)(F)c1ccc(C(=O)NCC(=O)N3CC4(C[C@H]3C(=O)N[C@H](C)c3cc(C(=N)N)cs3)OCCO4)cc1-2 ; C[C@@H](NC(=O)[C@@H]1CC2(CN1C(=O)CNC(=O)c1ccc3c(c1)-c1ccc(Cl)cc1C3(F)F)OCCO2)c1cc(C(=N)N)cs1] 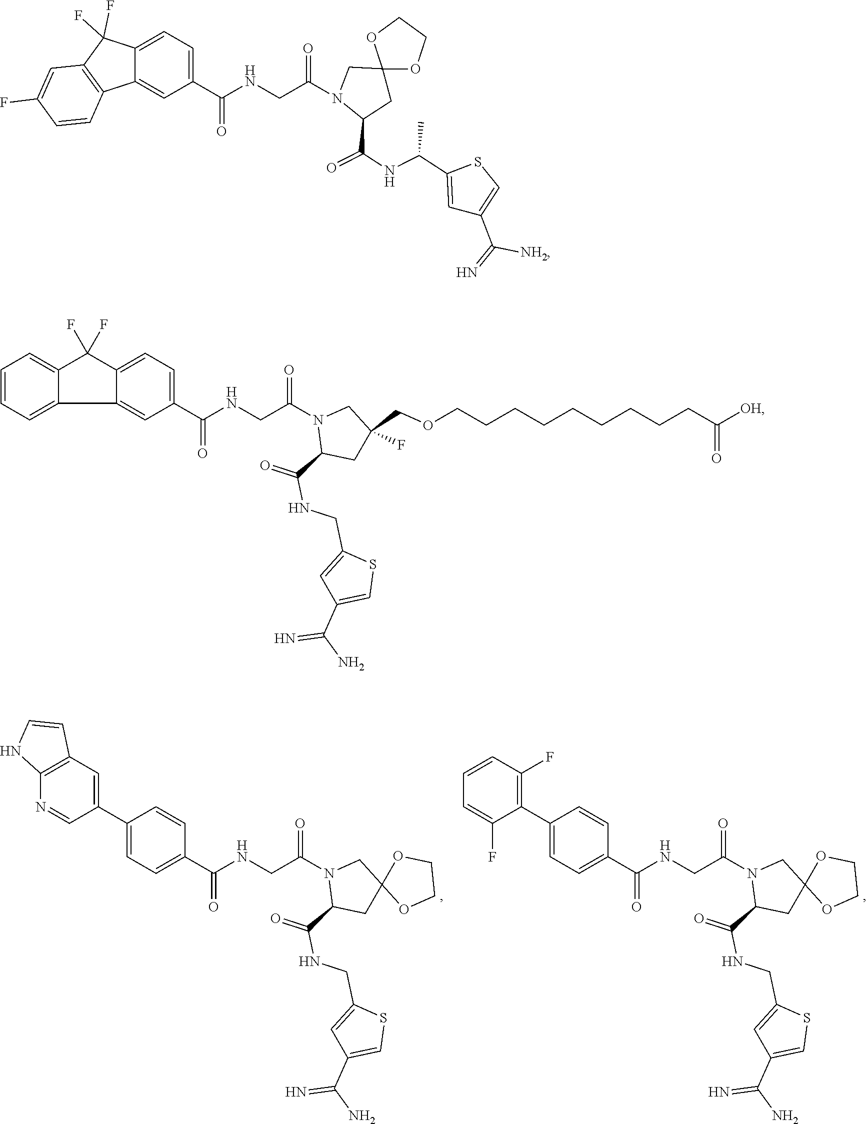 [SMILES: C[C@@H](NC(=O)[C@@H]1CC2(CN1C(=O)CNC(=O)c1ccc3c(c1)-c1ccc(F)cc1C3(F)F)OCCO2)c1cc(C(=N)N)cs1 ; N=C(N)c1csc(CNC(=O)[C@@H]2C[C@](F)(COCCCCCCCCCC(=O)O)CN2C(=O)CNC(=O)c2ccc3c(c2)-c2ccccc2C3(F)F)c1 ; N=C(N)c1csc(CNC(=O)[C@@H]2CC3(CN2C(=O)CNC(=O)c2ccc(-c4cnc5[nH]ccc5c4)cc2)OCCO3)c1 ; N=C(N)c1csc(CNC(=O)[C@@H]2CC3(CN2C(=O)CNC(=O)c2ccc(-c4c(F)cccc4F)cc2)OCCO3)c1] 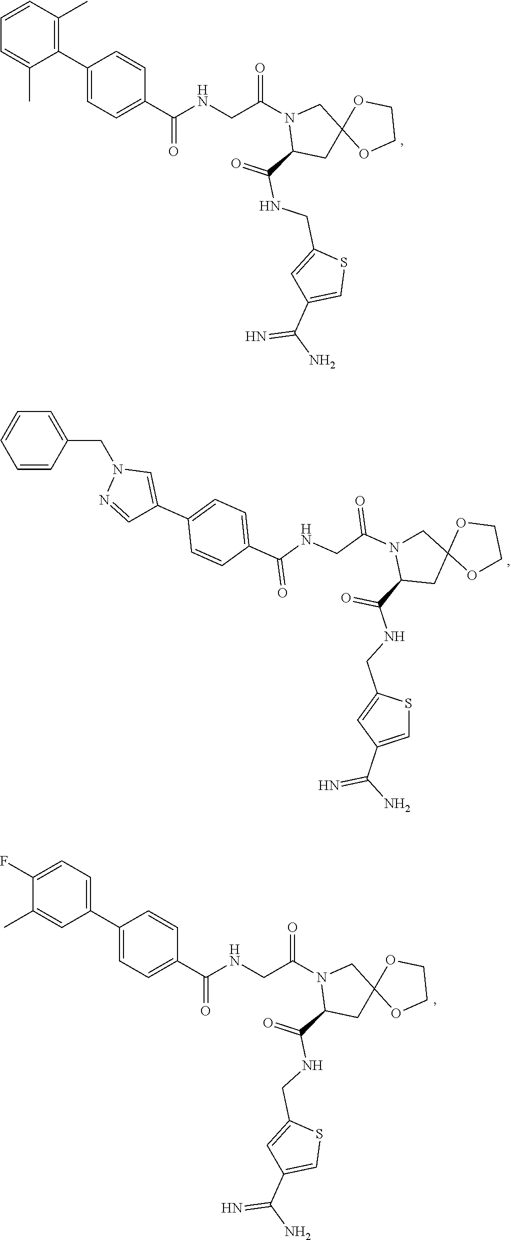 [SMILES: Cc1cccc(C)c1-c1ccc(C(=O)NCC(=O)N2CC3(C[C@H]2C(=O)NCc2cc(C(=N)N)cs2)OCCO3)cc1 ; N=C(N)c1csc(CNC(=O)[C@@H]2CC3(CN2C(=O)CNC(=O)c2ccc(-c4cnn(Cc5ccccc5)c4)cc2)OCCO3)c1 ; Cc1cc(-c2ccc(C(=O)NCC(=O)N3CC4(C[C@H]3C(=O)NCc3cc(C(=N)N)cs3)OCCO4)cc2)ccc1F] 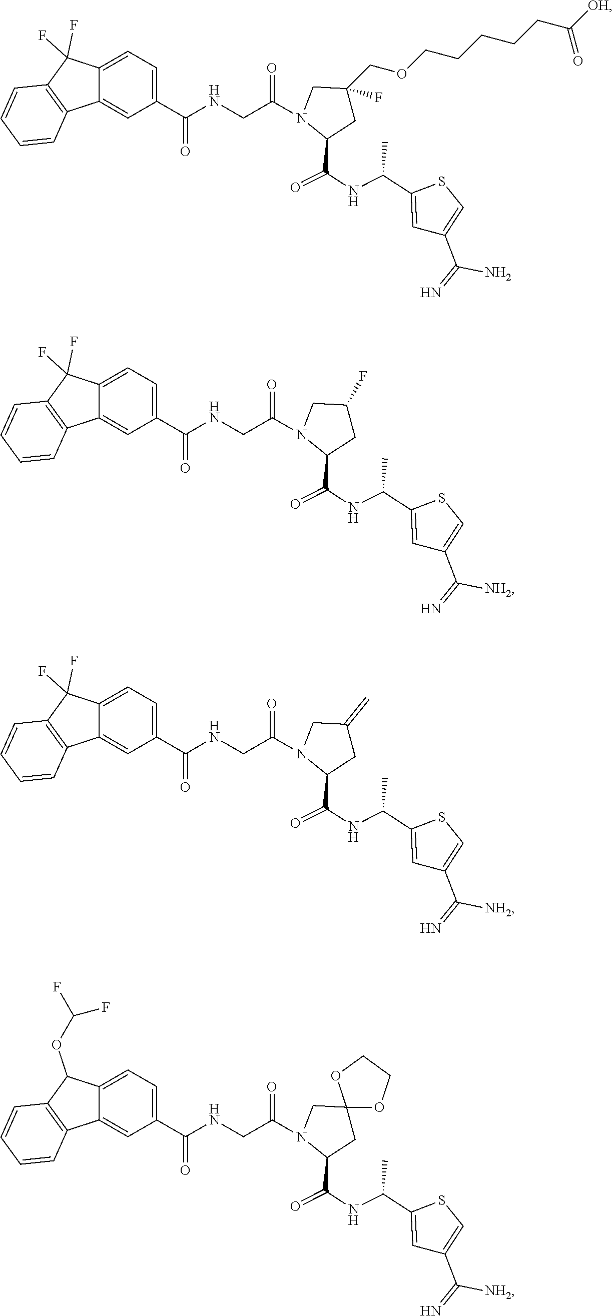 [SMILES: C[C@@H](NC(=O)[C@@H]1C[C@](F)(COCCCCCC(=O)O)CN1C(=O)CNC(=O)c1ccc2c(c1)-c1ccccc1C2(F)F)c1cc(C(=N)N)cs1 ; C[C@@H](NC(=O)[C@@H]1C[C@@H](F)CN1C(=O)CNC(=O)c1ccc2c(c1)-c1ccccc1C2(F)F)c1cc(C(=N)N)cs1 ; C=C1C[C@@H](C(=O)N[C@H](C)c2cc(C(=N)N)cs2)N(C(=O)CNC(=O)c2ccc3c(c2)-c2ccccc2C3(F)F)C1 ; C[C@@H](NC(=O)[C@@H]1CC2(CN1C(=O)CNC(=O)c1ccc3c(c1)-c1ccccc1C3OC(F)F)OCCO2)c1cc(C(=N)N)cs1] 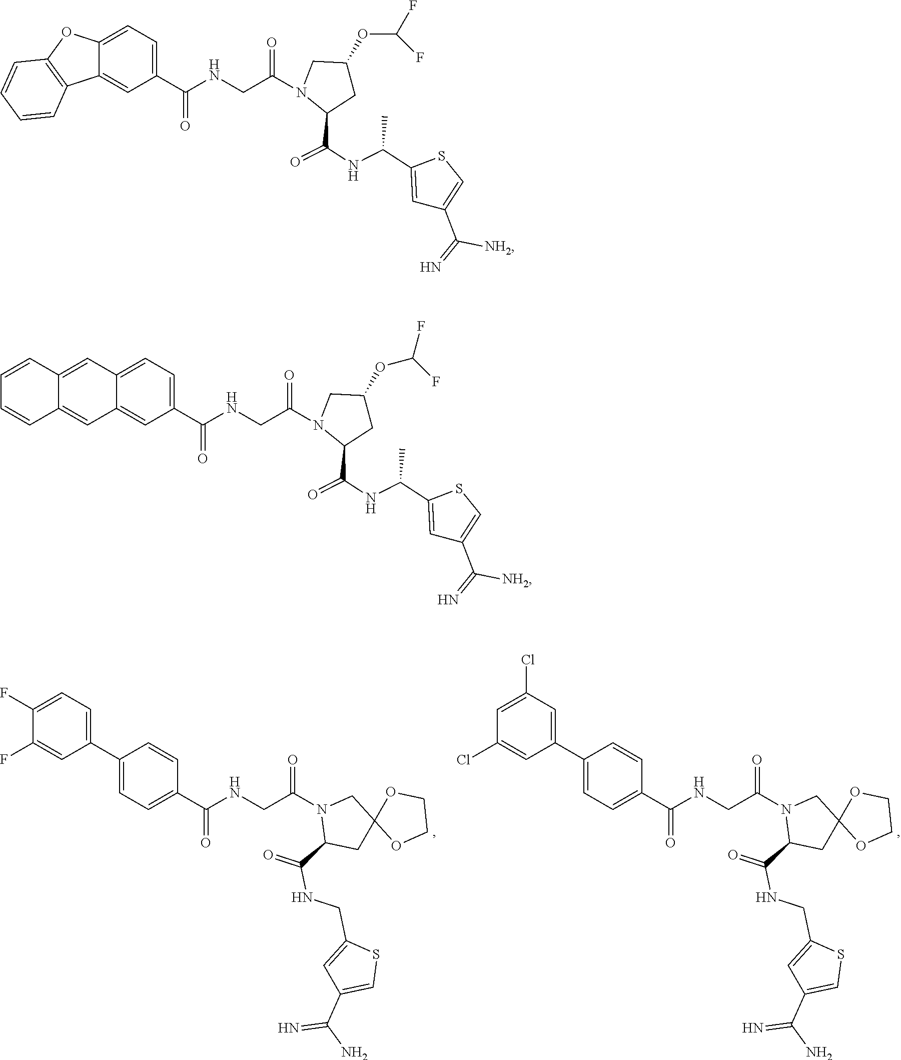 [SMILES: C[C@@H](NC(=O)[C@@H]1C[C@@H](OC(F)F)CN1C(=O)CNC(=O)c1ccc2oc3ccccc3c2c1)c1cc(C(=N)N)cs1 ; C[C@@H](NC(=O)[C@@H]1C[C@@H](OC(F)F)CN1C(=O)CNC(=O)c1ccc2cc3ccccc3cc2c1)c1cc(C(=N)N)cs1 ; N=C(N)c1csc(CNC(=O)[C@@H]2CC3(CN2C(=O)CNC(=O)c2ccc(-c4ccc(F)c(F)c4)cc2)OCCO3)c1 ; N=C(N)c1csc(CNC(=O)[C@@H]2CC3(CN2C(=O)CNC(=O)c2ccc(-c4cc(Cl)cc(Cl)c4)cc2)OCCO3)c1] 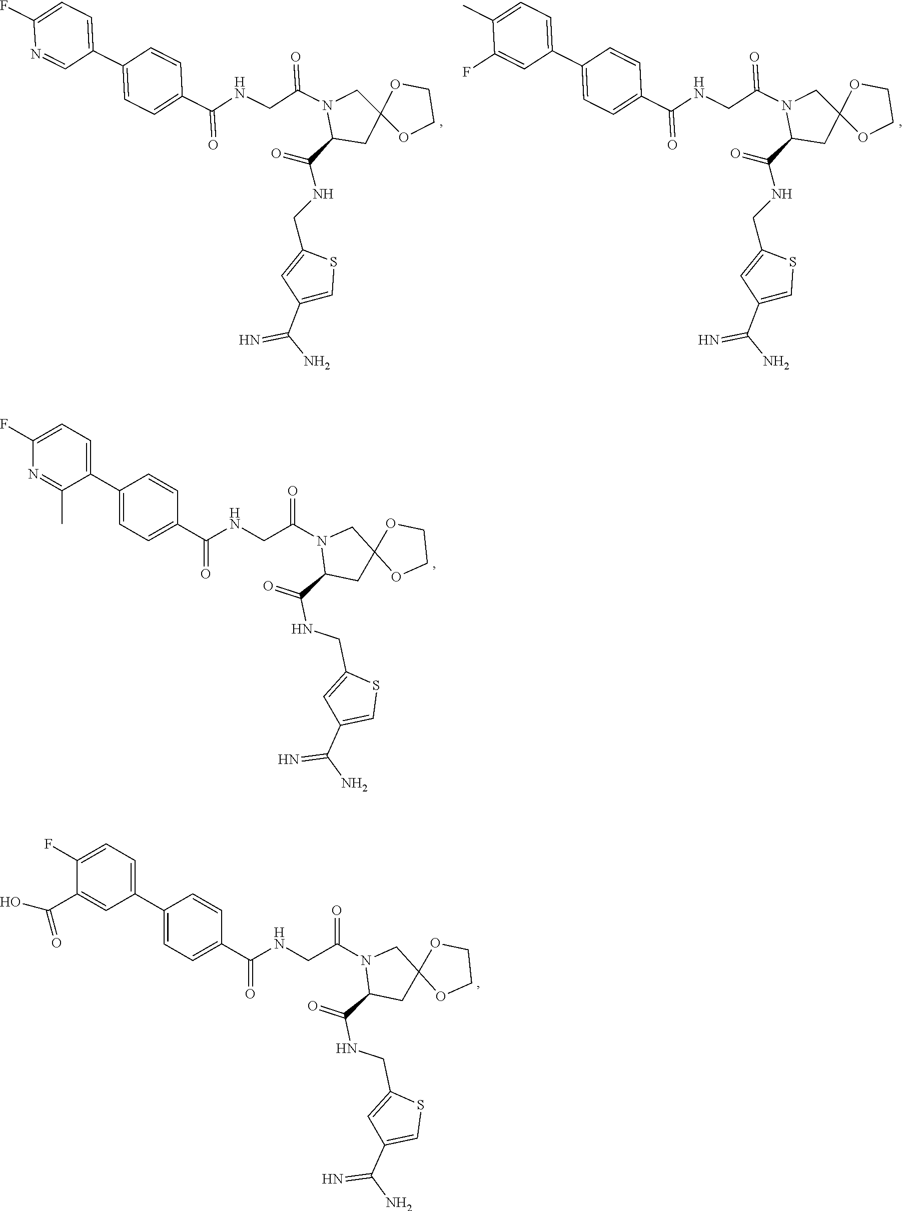 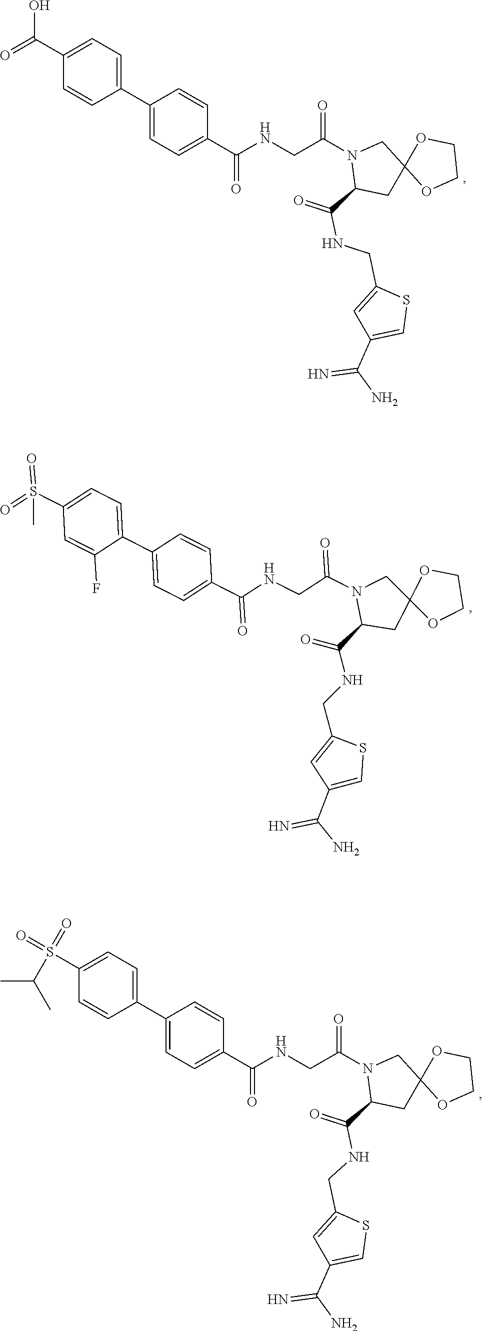 [SMILES: N=C(N)c1csc(CNC(=O)[C@@H]2CC3(CN2C(=O)CNC(=O)c2ccc(-c4ccc(C(=O)O)cc4)cc2)OCCO3)c1 ; CS(=O)(=O)c1ccc(-c2ccc(C(=O)NCC(=O)N3CC4(C[C@H]3C(=O)NCc3cc(C(=N)N)cs3)OCCO4)cc2)c(F)c1 ; CC(C)S(=O)(=O)c1ccc(-c2ccc(C(=O)NCC(=O)N3CC4(C[C@H]3C(=O)NCc3cc(C(=N)N)cs3)OCCO4)cc2)cc1] 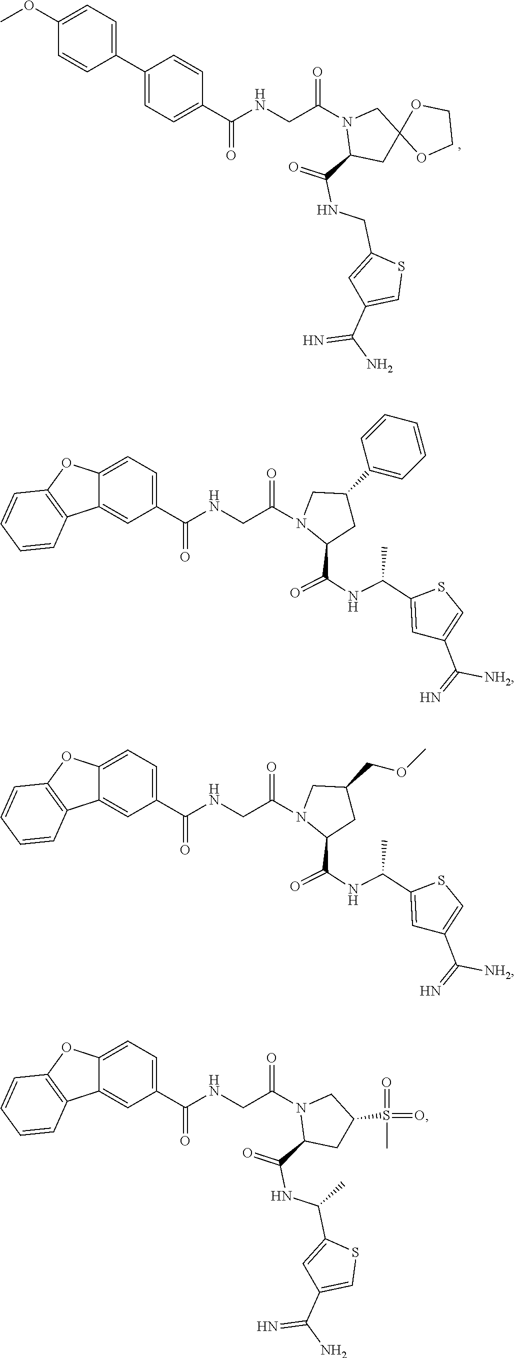 [SMILES: COc1ccc(-c2ccc(C(=O)NCC(=O)N3CC4(C[C@H]3C(=O)NCc3cc(C(=N)N)cs3)OCCO4)cc2)cc1 ; C[C@@H](NC(=O)[C@@H]1C[C@@H](c2ccccc2)CN1C(=O)CNC(=O)c1ccc2oc3ccccc3c2c1)c1cc(C(=N)N)cs1 ; COC[C@H]1C[C@@H](C(=O)N[C@H](C)c2cc(C(=N)N)cs2)N(C(=O)CNC(=O)c2ccc3oc4ccccc4c3c2)C1 ; C[C@@H](NC(=O)[C@@H]1C[C@@H](S(C)(=O)=O)CN1C(=O)CNC(=O)c1ccc2oc3ccccc3c2c1)c1cc(C(=N)N)cs1] 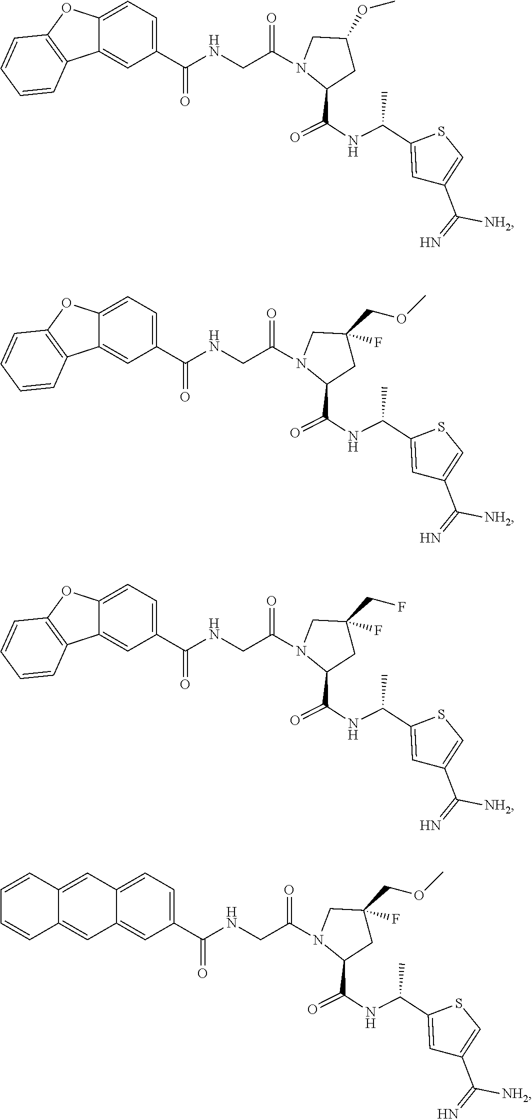 [SMILES: CO[C@@H]1C[C@@H](C(=O)N[C@H](C)c2cc(C(=N)N)cs2)N(C(=O)CNC(=O)c2ccc3oc4ccccc4c3c2)C1 ; COC[C@@]1(F)C[C@@H](C(=O)N[C@H](C)c2cc(C(=N)N)cs2)N(C(=O)CNC(=O)c2ccc3oc4ccccc4c3c2)C1 ; C[C@@H](NC(=O)[C@@H]1C[C@](F)(CF)CN1C(=O)CNC(=O)c1ccc2oc3ccccc3c2c1)c1cc(C(=N)N)cs1 ; COC[C@@]1(F)C[C@@H](C(=O)N[C@H](C)c2cc(C(=N)N)cs2)N(C(=O)CNC(=O)c2ccc3cc4ccccc4cc3c2)C1] 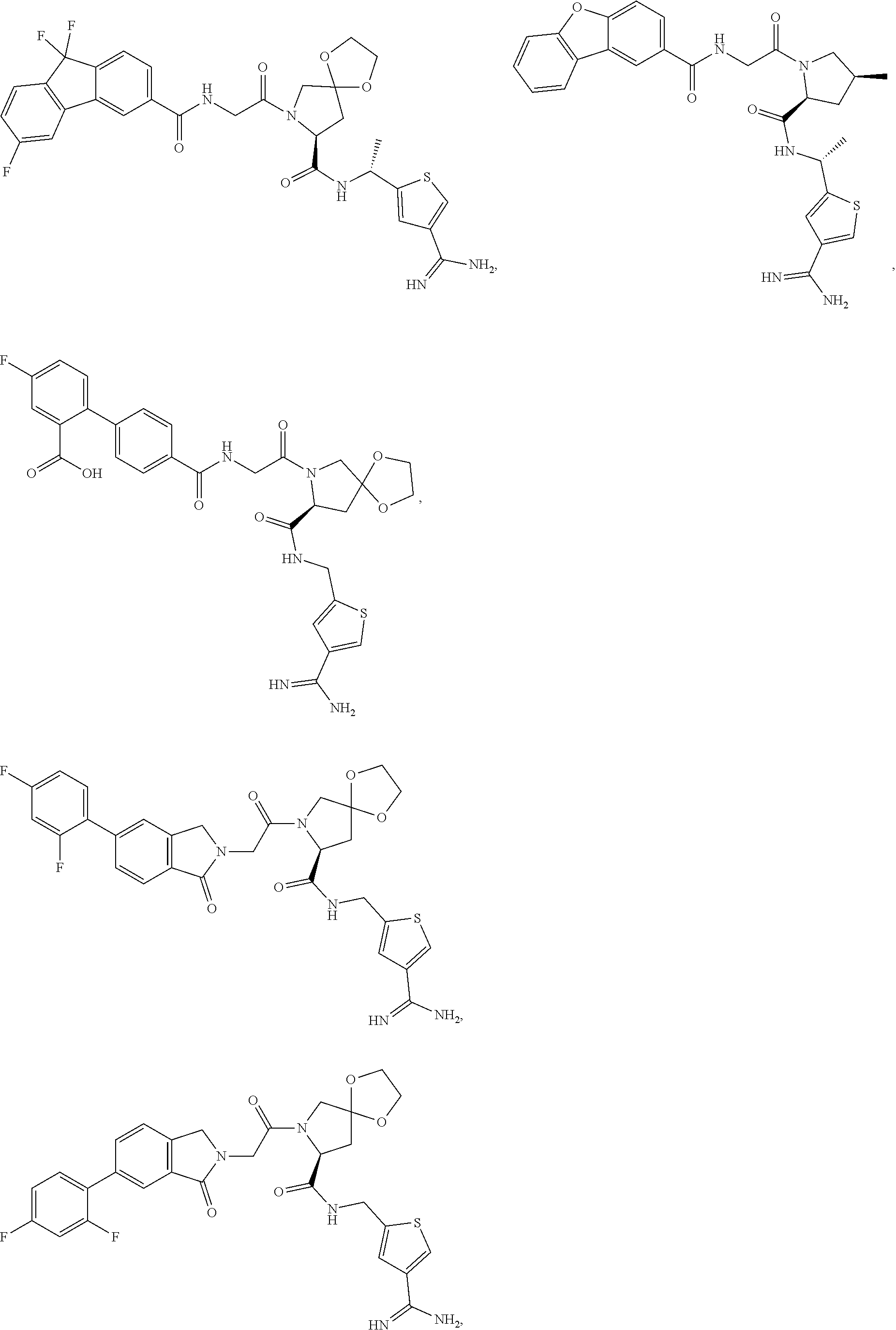 [SMILES: C[C@@H](NC(=O)[C@@H]1CC2(CN1C(=O)CNC(=O)c1ccc3c(c1)-c1cc(F)ccc1C3(F)F)OCCO2)c1cc(C(=N)N)cs1 ; C[C@H]1C[C@@H](C(=O)N[C@H](C)c2cc(C(=N)N)cs2)N(C(=O)CNC(=O)c2ccc3oc4ccccc4c3c2)C1 ; N=C(N)c1csc(CNC(=O)[C@@H]2CC3(CN2C(=O)CNC(=O)c2ccc(-c4ccc(F)cc4C(=O)O)cc2)OCCO3)c1 ; N=C(N)c1csc(CNC(=O)[C@@H]2CC3(CN2C(=O)CN2Cc4cc(-c5ccc(F)cc5F)ccc4C2=O)OCCO3)c1 ; N=C(N)c1csc(CNC(=O)[C@@H]2CC3(CN2C(=O)CN2Cc4ccc(-c5ccc(F)cc5F)cc4C2=O)OCCO3)c1] 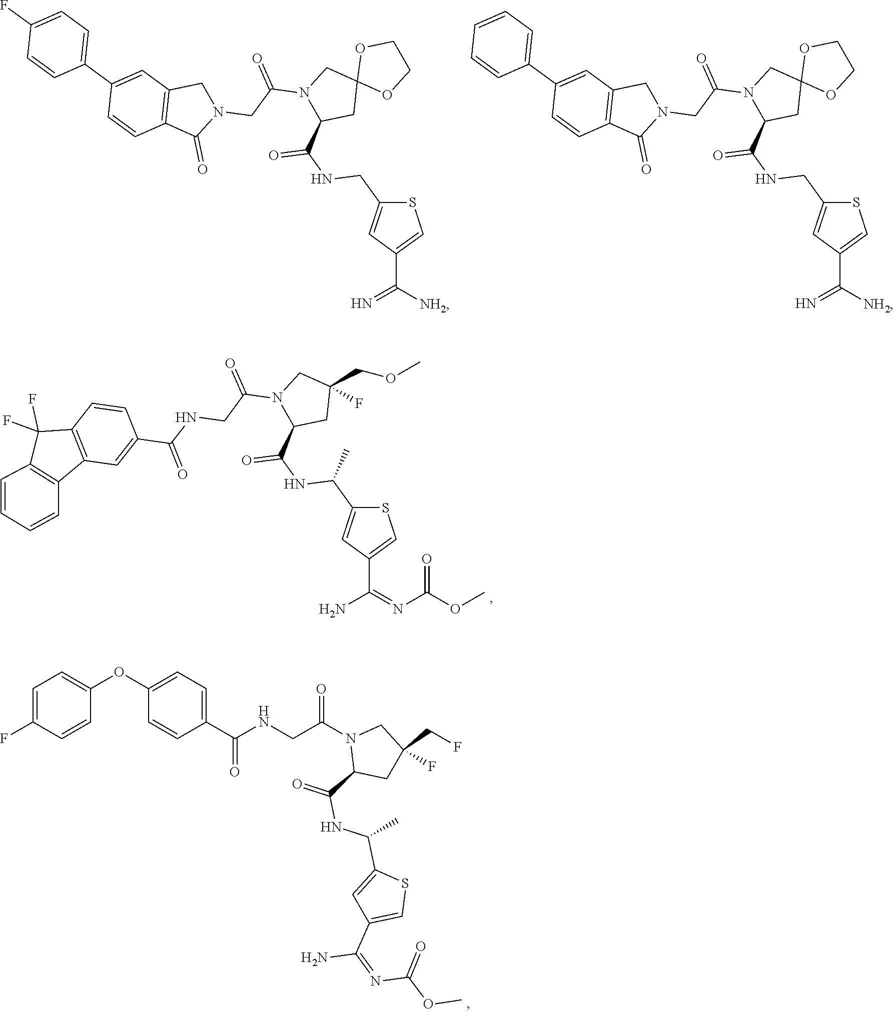 [SMILES: N=C(N)c1csc(CNC(=O)[C@@H]2CC3(CN2C(=O)CN2Cc4cc(-c5ccc(F)cc5)ccc4C2=O)OCCO3)c1 ; N=C(N)c1csc(CNC(=O)[C@@H]2CC3(CN2C(=O)CN2Cc4cc(-c5ccccc5)ccc4C2=O)OCCO3)c1 ; COC[C@@]1(F)C[C@@H](C(=O)N[C@H](C)c2cc(/C(N)=N\C(=O)OC)cs2)N(C(=O)CNC(=O)c2ccc3c(c2)-c2ccccc2C3(F)F)C1 ; COC(=O)/N=C(/N)c1csc([C@@H](C)NC(=O)[C@@H]2C[C@](F)(CF)CN2C(=O)CNC(=O)c2ccc(Oc3ccc(F)cc3)cc2)c1] 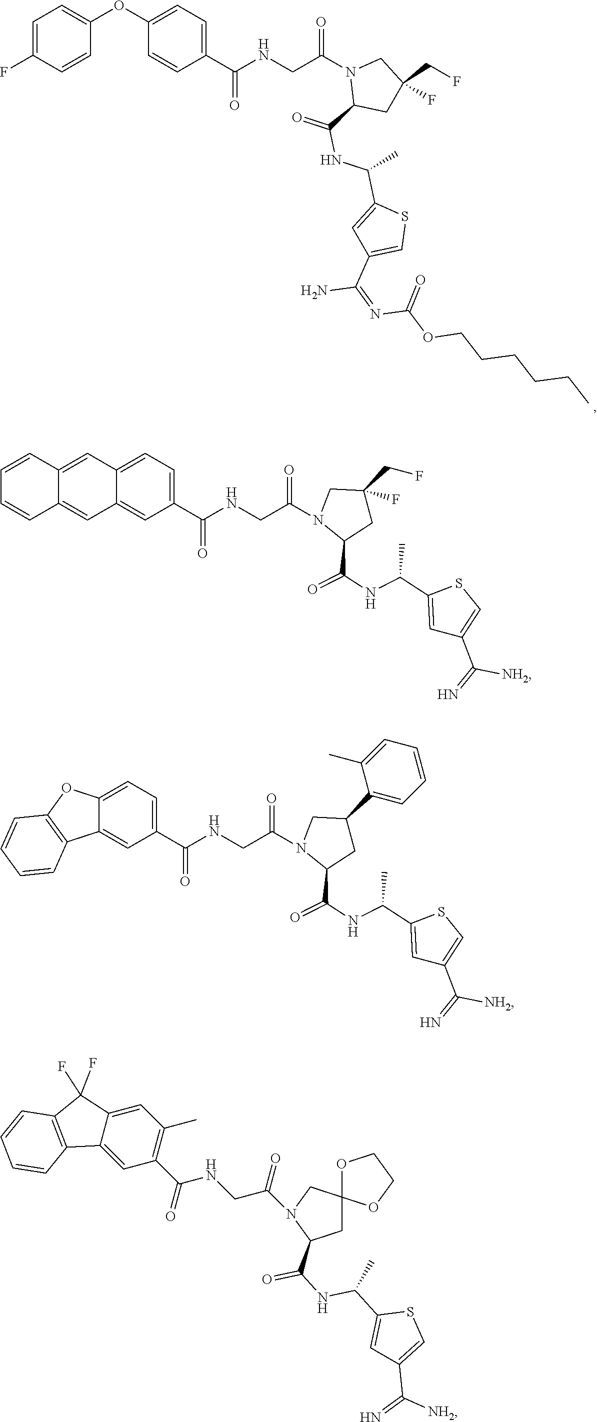 [SMILES: CCCCCCOC(=O)/N=C(/N)c1csc([C@@H](C)NC(=O)[C@@H]2C[C@](F)(CF)CN2C(=O)CNC(=O)c2ccc(Oc3ccc(F)cc3)cc2)c1 ; C[C@@H](NC(=O)[C@@H]1C[C@](F)(CF)CN1C(=O)CNC(=O)c1ccc2cc3ccccc3cc2c1)c1cc(C(=N)N)cs1 ; Cc1ccccc1[C@H]1C[C@@H](C(=O)N[C@H](C)c2cc(C(=N)N)cs2)N(C(=O)CNC(=O)c2ccc3oc4ccccc4c3c2)C1 ; Cc1cc2c(cc1C(=O)NCC(=O)N1CC3(C[C@H]1C(=O)N[C@H](C)c1cc(C(=N)N)cs1)OCCO3)-c1ccccc1C2(F)F] 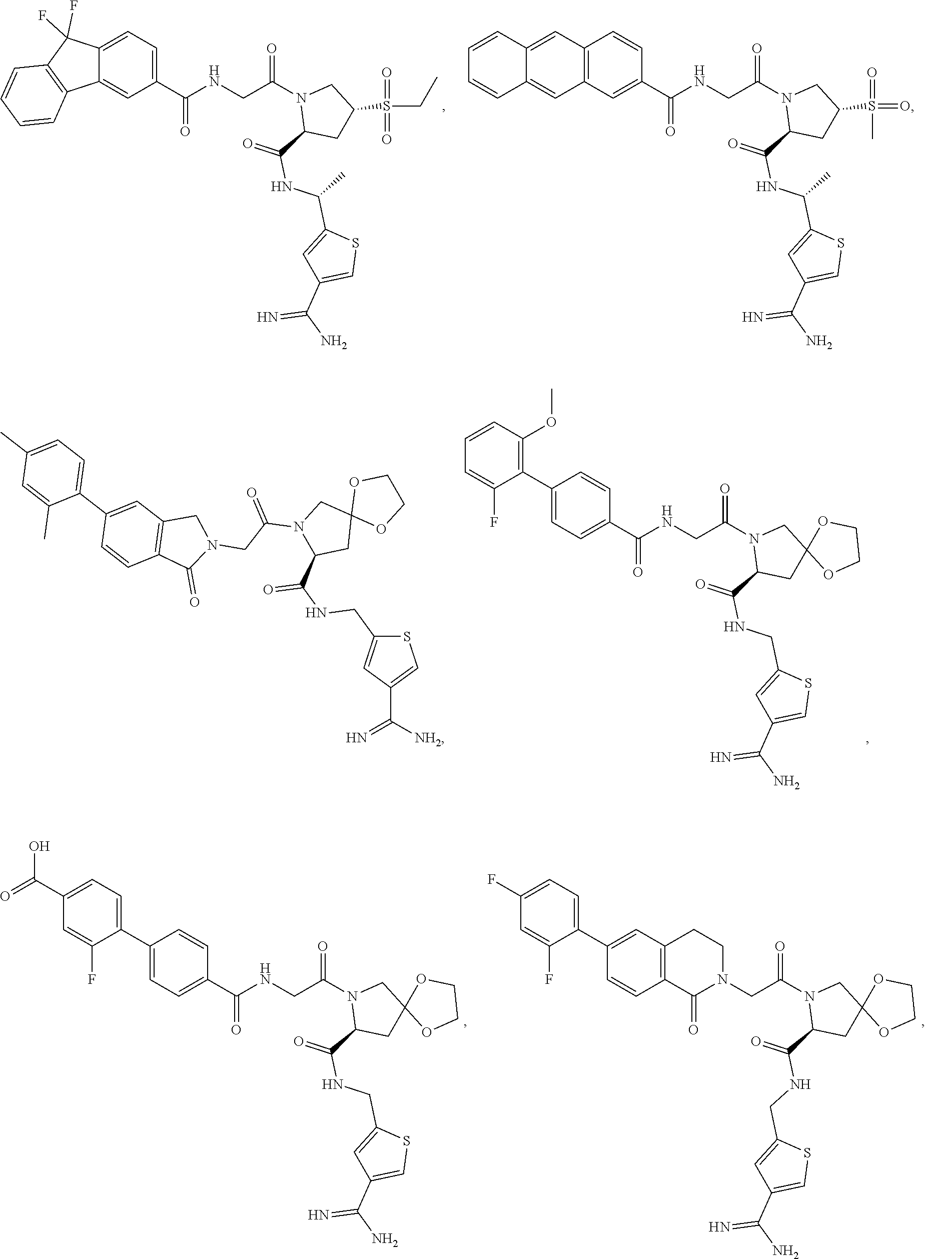 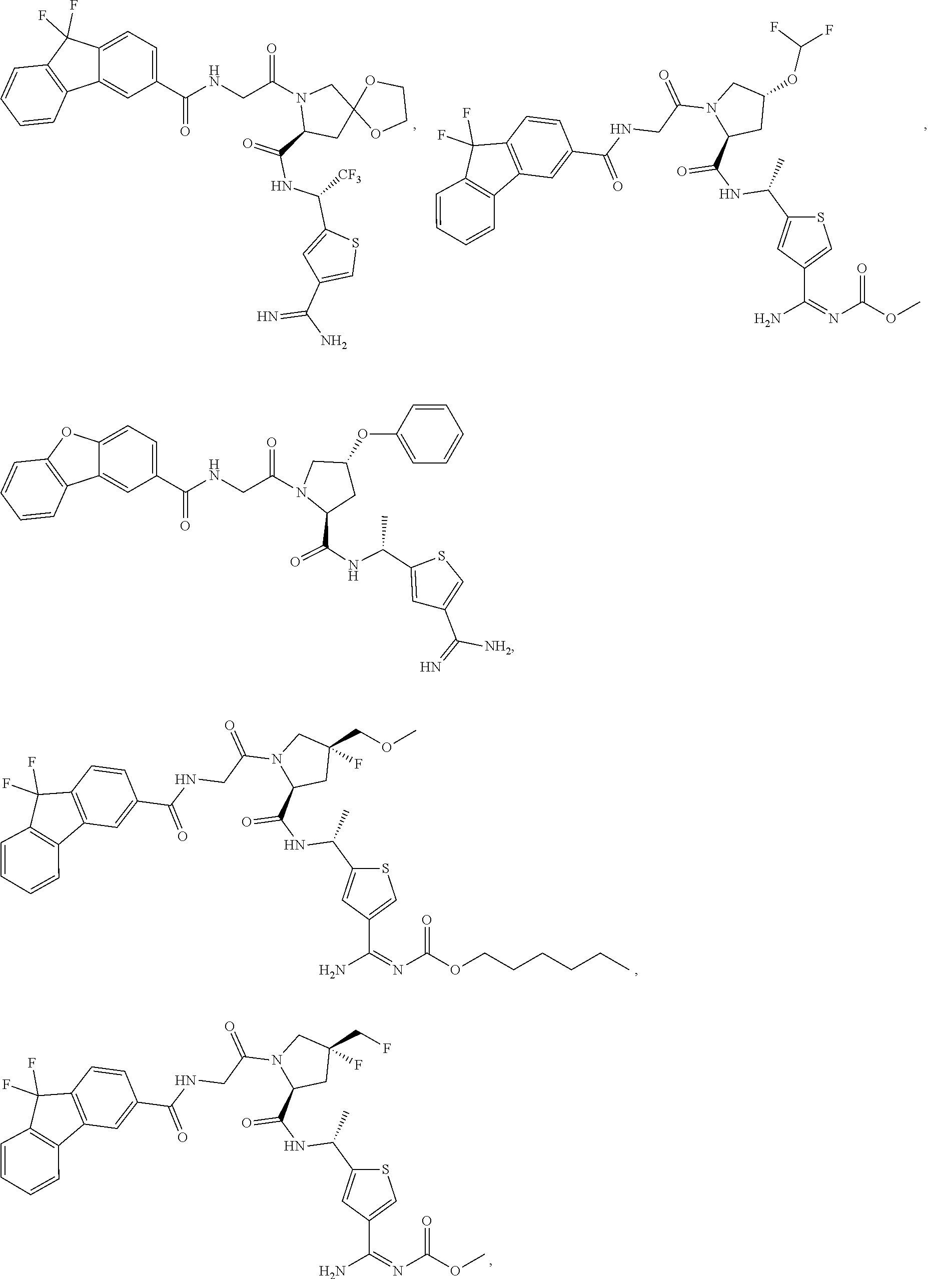 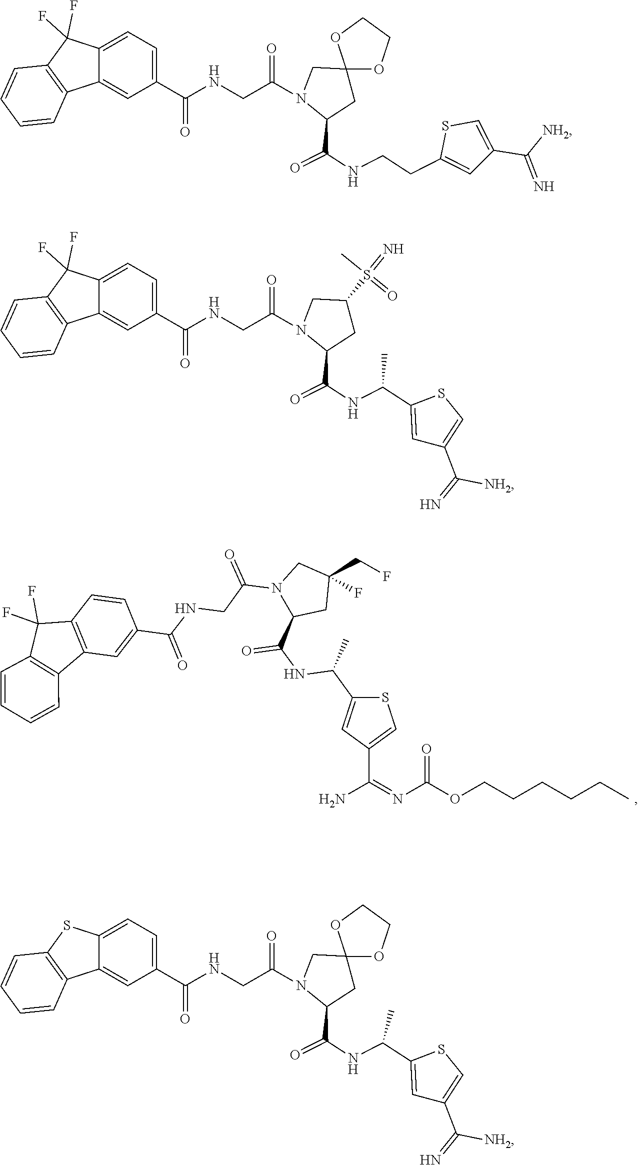 [SMILES: N=C(N)c1csc(CCNC(=O)[C@@H]2CC3(CN2C(=O)CNC(=O)c2ccc4c(c2)-c2ccccc2C4(F)F)OCCO3)c1 ; C[C@@H](NC(=O)[C@@H]1C[C@@H](S(C)(=N)=O)CN1C(=O)CNC(=O)c1ccc2c(c1)-c1ccccc1C2(F)F)c1cc(C(=N)N)cs1 ; CCCCCCOC(=O)/N=C(/N)c1csc([C@@H](C)NC(=O)[C@@H]2C[C@](F)(CF)CN2C(=O)CNC(=O)c2ccc3c(c2)-c2ccccc2C3(F)F)c1 ; C[C@@H](NC(=O)[C@@H]1CC2(CN1C(=O)CNC(=O)c1ccc3sc4ccccc4c3c1)OCCO2)c1cc(C(=N)N)cs1] 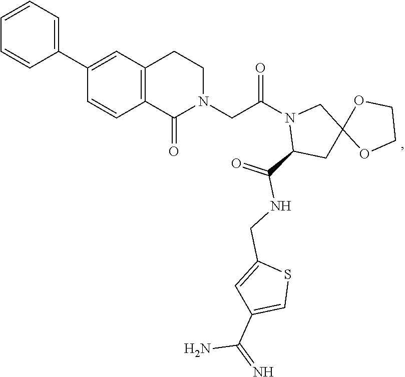 [SMILES: N=C(N)c1csc(CNC(=O)[C@@H]2CC3(CN2C(=O)CN2CCc4cc(-c5ccccc5)ccc4C2=O)OCCO3)c1] 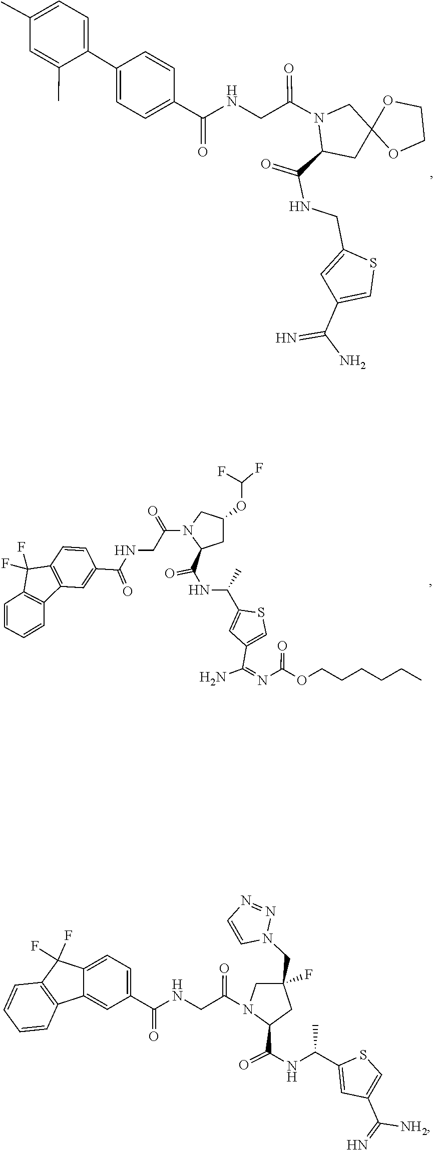 [SMILES: Cc1ccc(-c2ccc(C(=O)NCC(=O)N3CC4(C[C@H]3C(=O)NCc3cc(C(=N)N)cs3)OCCO4)cc2)c(C)c1 ; CCCCCCOC(=O)/N=C(/N)c1csc([C@@H](C)NC(=O)[C@@H]2C[C@@H](OC(F)F)CN2C(=O)CNC(=O)c2ccc3c(c2)-c2ccccc2C3(F)F)c1 ; C[C@@H](NC(=O)[C@@H]1C[C@](F)(Cn2ccnn2)CN1C(=O)CNC(=O)c1ccc2c(c1)-c1ccccc1C2(F)F)c1cc(C(=N)N)cs1] 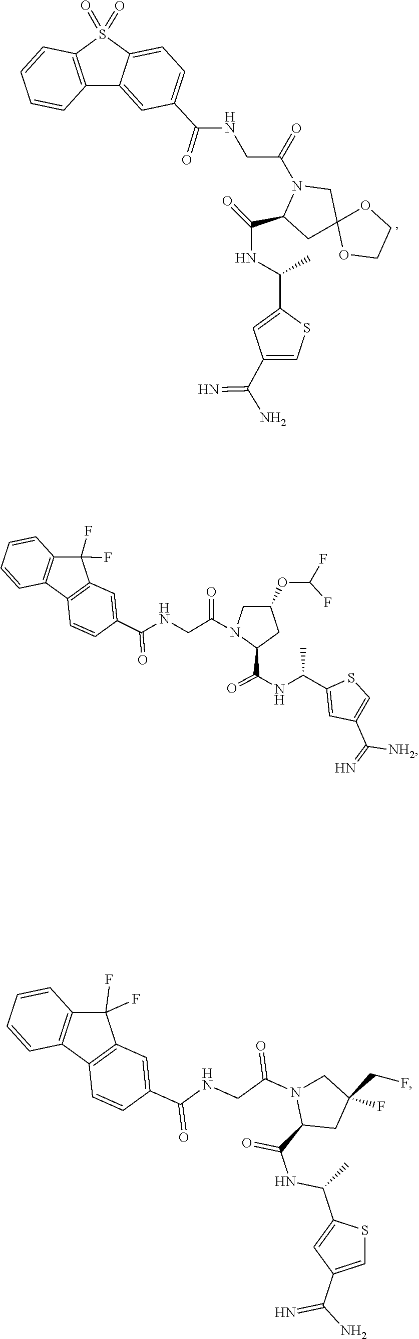 [SMILES: C[C@@H](NC(=O)[C@@H]1CC2(CN1C(=O)CNC(=O)c1ccc3c(c1)-c1ccccc1S3(=O)=O)OCCO2)c1cc(C(=N)N)cs1 ; C[C@@H](NC(=O)[C@@H]1C[C@@H](OC(F)F)CN1C(=O)CNC(=O)c1ccc2c(c1)C(F)(F)c1ccccc1-2)c1cc(C(=N)N)cs1 ; C[C@@H](NC(=O)[C@@H]1C[C@](F)(CF)CN1C(=O)CNC(=O)c1ccc2c(c1)C(F)(F)c1ccccc1-2)c1cc(C(=N)N)cs1] 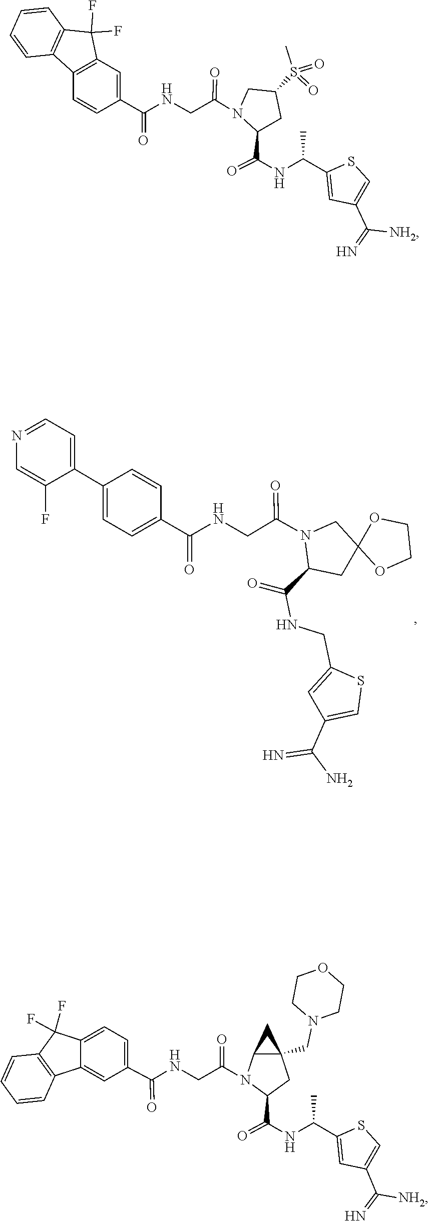 [SMILES: C[C@@H](NC(=O)[C@@H]1C[C@@H](S(C)(=O)=O)CN1C(=O)CNC(=O)c1ccc2c(c1)C(F)(F)c1ccccc1-2)c1cc(C(=N)N)cs1 ; N=C(N)c1csc(CNC(=O)[C@@H]2CC3(CN2C(=O)CNC(=O)c2ccc(-c4ccncc4F)cc2)OCCO3)c1 ; C[C@@H](NC(=O)[C@@H]1C[C@]2(CN3CCOCC3)C[C@@H]2N1C(=O)CNC(=O)c1ccc2c(c1)-c1ccccc1C2(F)F)c1cc(C(=N)N)cs1] 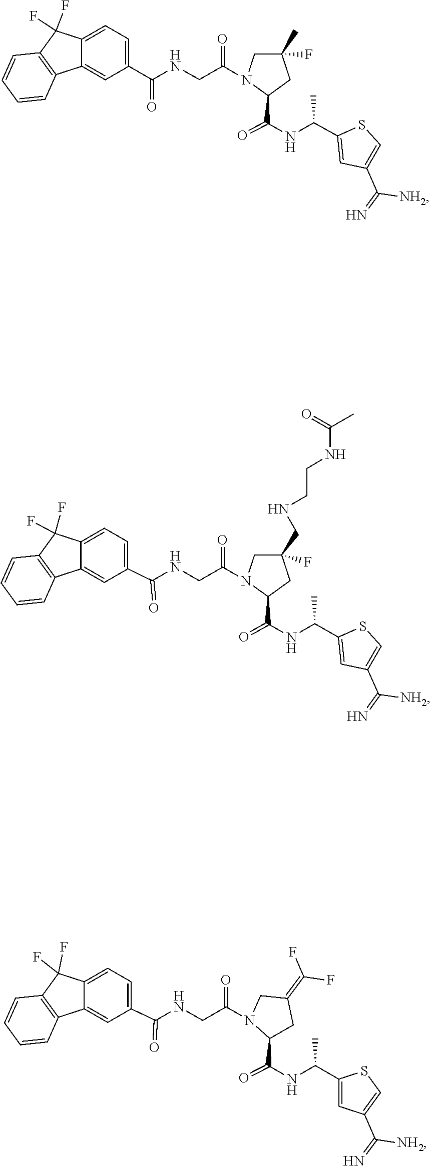 [SMILES: C[C@@H](NC(=O)[C@@H]1C[C@@](C)(F)CN1C(=O)CNC(=O)c1ccc2c(c1)-c1ccccc1C2(F)F)c1cc(C(=N)N)cs1 ; CC(=O)NCCNC[C@@]1(F)C[C@@H](C(=O)N[C@H](C)c2cc(C(=N)N)cs2)N(C(=O)CNC(=O)c2ccc3c(c2)-c2ccccc2C3(F)F)C1 ; C[C@@H](NC(=O)[C@@H]1CC(=C(F)F)CN1C(=O)CNC(=O)c1ccc2c(c1)-c1ccccc1C2(F)F)c1cc(C(=N)N)cs1] 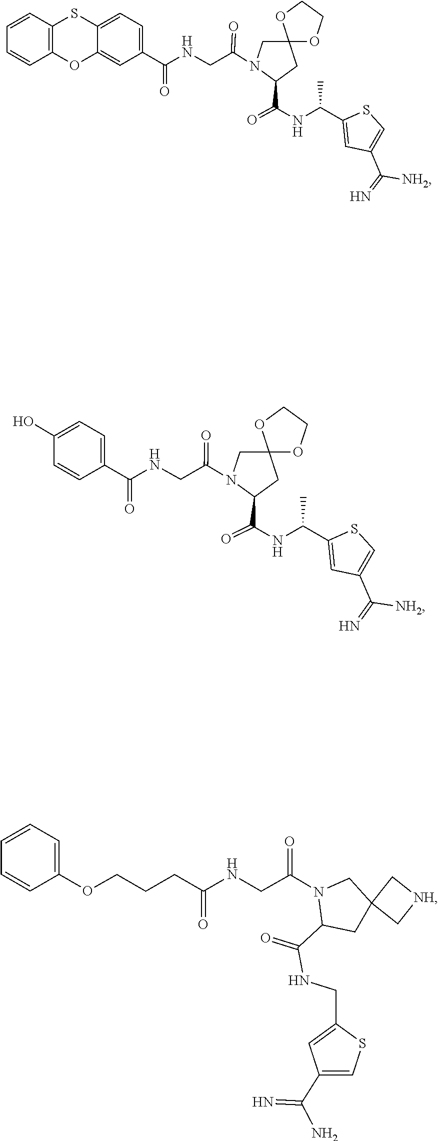 [SMILES: C[C@@H](NC(=O)[C@@H]1CC2(CN1C(=O)CNC(=O)c1ccc3c(c1)Oc1ccccc1S3)OCCO2)c1cc(C(=N)N)cs1 ; C[C@@H](NC(=O)[C@@H]1CC2(CN1C(=O)CNC(=O)c1ccc(O)cc1)OCCO2)c1cc(C(=N)N)cs1 ; N=C(N)c1csc(CNC(=O)C2CC3(CNC3)CN2C(=O)CNC(=O)CCCOc2ccccc2)c1] 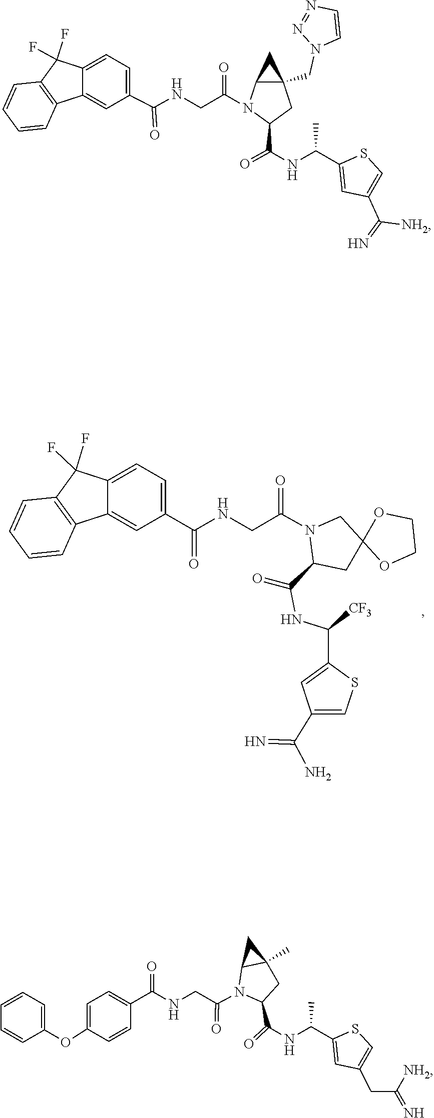 [SMILES: C[C@@H](NC(=O)[C@@H]1C[C@]2(Cn3ccnn3)C[C@@H]2N1C(=O)CNC(=O)c1ccc2c(c1)-c1ccccc1C2(F)F)c1cc(C(=N)N)cs1 ; N=C(N)c1csc([C@@H](NC(=O)[C@@H]2CC3(CN2C(=O)CNC(=O)c2ccc4c(c2)-c2ccccc2C4(F)F)OCCO3)C(F)(F)F)c1 ; C[C@@H](NC(=O)[C@@H]1C[C@]2(C)C[C@@H]2N1C(=O)CNC(=O)c1ccc(Oc2ccccc2)cc1)c1cc(CC(=N)N)cs1] 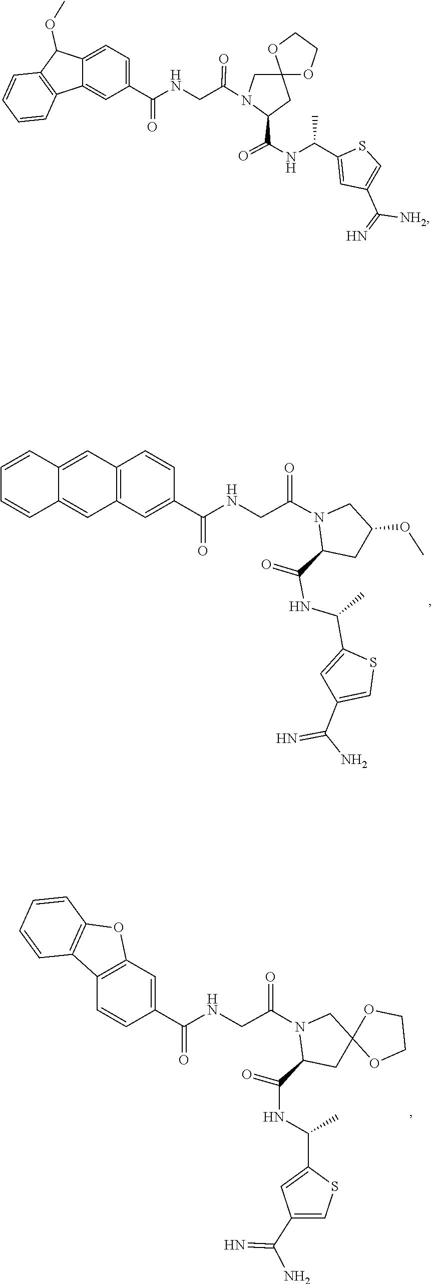 [SMILES: COC1c2ccccc2-c2cc(C(=O)NCC(=O)N3CC4(C[C@H]3C(=O)N[C@H](C)c3cc(C(=N)N)cs3)OCCO4)ccc21 ; CO[C@@H]1C[C@@H](C(=O)N[C@H](C)c2cc(C(=N)N)cs2)N(C(=O)CNC(=O)c2ccc3cc4ccccc4cc3c2)C1 ; C[C@@H](NC(=O)[C@@H]1CC2(CN1C(=O)CNC(=O)c1ccc3c(c1)oc1ccccc13)OCCO2)c1cc(C(=N)N)cs1] 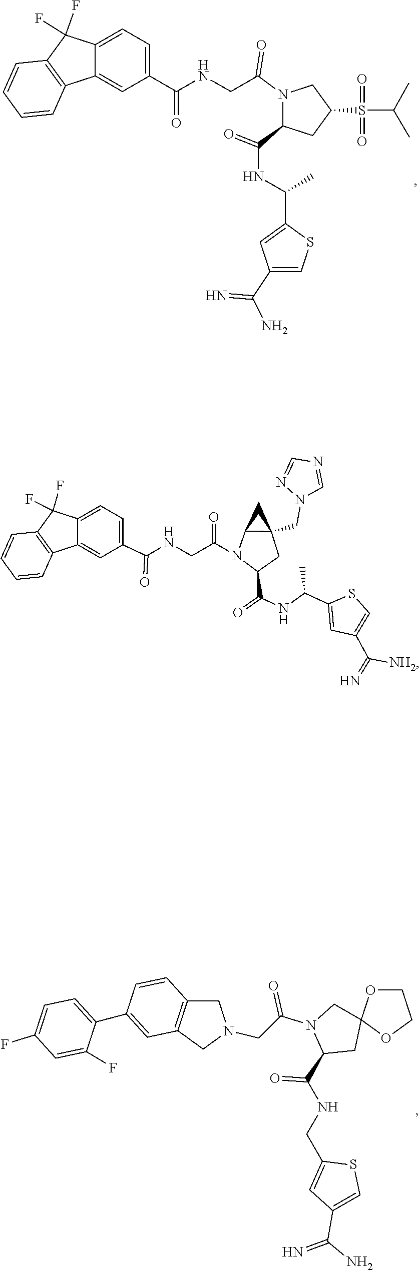 [SMILES: CC(C)S(=O)(=O)[C@@H]1C[C@@H](C(=O)N[C@H](C)c2cc(C(=N)N)cs2)N(C(=O)CNC(=O)c2ccc3c(c2)-c2ccccc2C3(F)F)C1 ; C[C@@H](NC(=O)[C@@H]1C[C@]2(Cn3cncn3)C[C@@H]2N1C(=O)CNC(=O)c1ccc2c(c1)-c1ccccc1C2(F)F)c1cc(C(=N)N)cs1 ; N=C(N)c1csc(CNC(=O)[C@@H]2CC3(CN2C(=O)CN2Cc4ccc(-c5ccc(F)cc5F)cc4C2)OCCO3)c1] 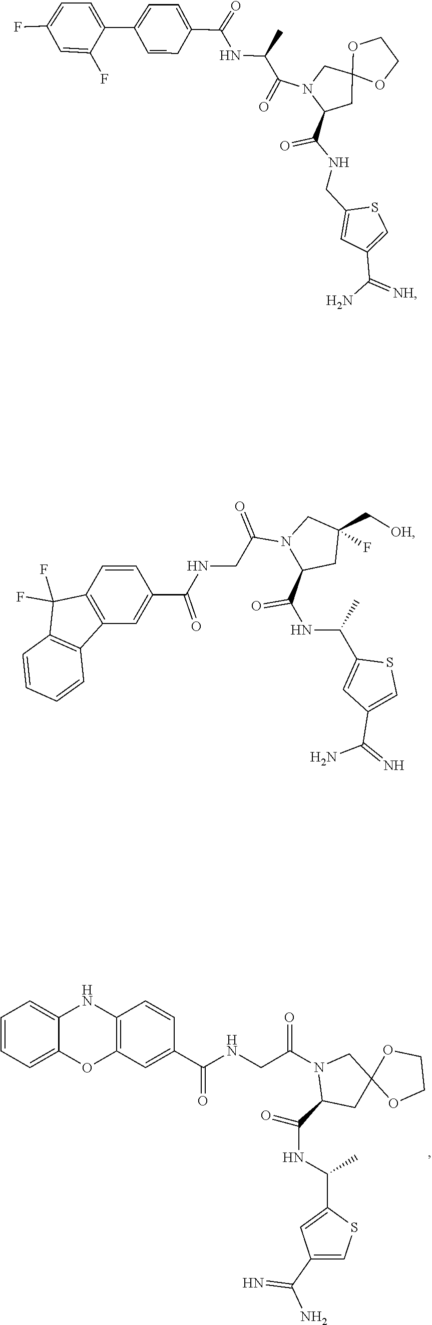 [SMILES: C[C@H](NC(=O)C1=CC=C(c2ccc(F)cc2F)C=CC1)C(=O)N1CC2(C[C@H]1C(=O)NCc1cc(C(=N)N)cs1)OCCO2 ; C[C@@H](NC(=O)[C@@H]1C[C@](F)(CO)CN1C(=O)CNC(=O)c1ccc2c(c1)-c1ccccc1C2(F)F)c1cc(C(=N)N)cs1 ; C[C@@H](NC(=O)[C@@H]1CC2(CN1C(=O)CNC(=O)c1ccc3c(c1)Oc1ccccc1N3)OCCO2)c1cc(C(=N)N)cs1] 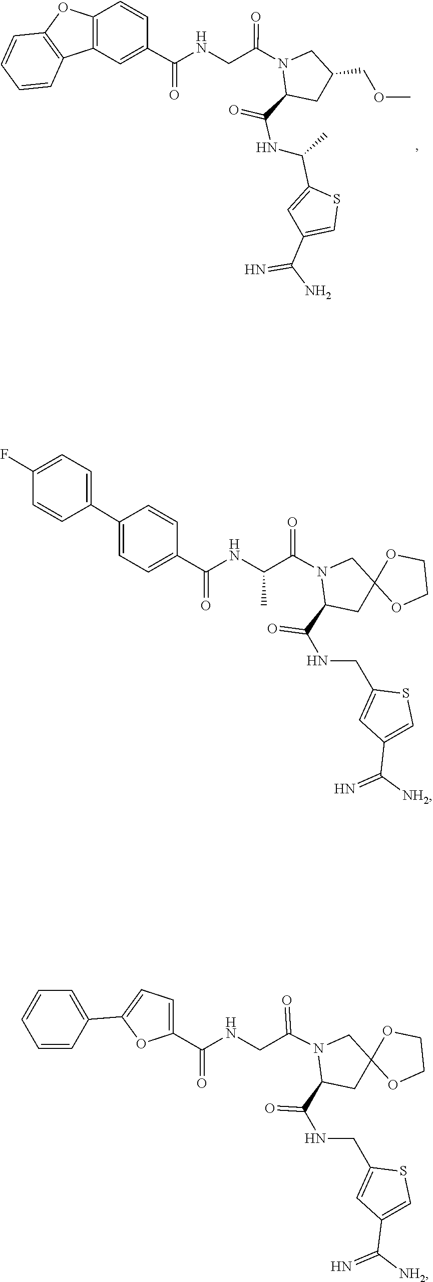 [SMILES: COC[C@@H]1C[C@@H](C(=O)N[C@H](C)c2cc(C(=N)N)cs2)N(C(=O)CNC(=O)c2ccc3oc4ccccc4c3c2)C1 ; C[C@H](NC(=O)c1ccc(-c2ccc(F)cc2)cc1)C(=O)N1CC2(C[C@H]1C(=O)NCc1cc(C(=N)N)cs1)OCCO2 ; N=C(N)c1csc(CNC(=O)[C@@H]2CC3(CN2C(=O)CNC(=O)c2ccc(-c4ccccc4)o2)OCCO3)c1] 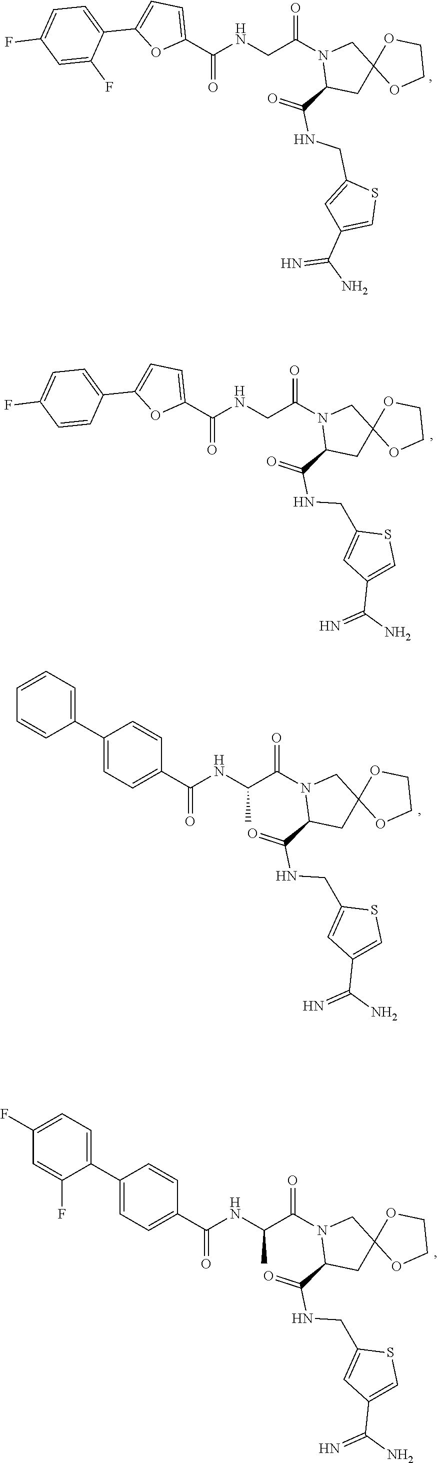 [SMILES: N=C(N)c1csc(CNC(=O)[C@@H]2CC3(CN2C(=O)CNC(=O)c2ccc(-c4ccc(F)cc4F)o2)OCCO3)c1 ; N=C(N)c1csc(CNC(=O)[C@@H]2CC3(CN2C(=O)CNC(=O)c2ccc(-c4ccc(F)cc4)o2)OCCO3)c1 ; C[C@H](NC(=O)c1ccc(-c2ccccc2)cc1)C(=O)N1CC2(C[C@H]1C(=O)NCc1cc(C(=N)N)cs1)OCCO2 ; C[C@@H](NC(=O)c1ccc(-c2ccc(F)cc2F)cc1)C(=O)N1CC2(C[C@H]1C(=O)NCc1cc(C(=N)N)cs1)OCCO2] 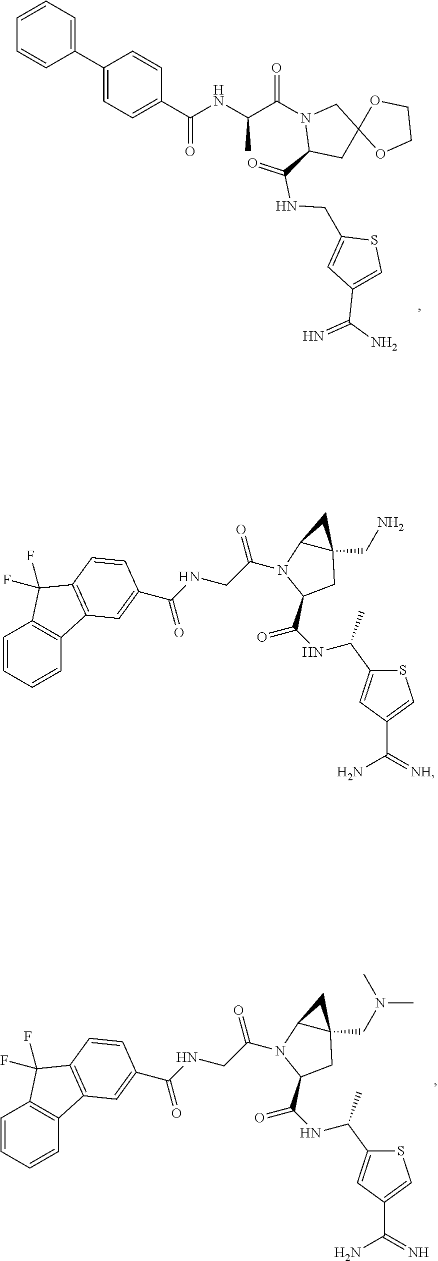 [SMILES: C[C@@H](NC(=O)c1ccc(-c2ccccc2)cc1)C(=O)N1CC2(C[C@H]1C(=O)NCc1cc(C(=N)N)cs1)OCCO2 ; C[C@@H](NC(=O)[C@@H]1C[C@]2(CN)C[C@@H]2N1C(=O)CNC(=O)c1ccc2c(c1)-c1ccccc1C2(F)F)c1cc(C(=N)N)cs1 ; C[C@@H](NC(=O)[C@@H]1C[C@]2(CN(C)C)C[C@@H]2N1C(=O)CNC(=O)c1ccc2c(c1)-c1ccccc1C2(F)F)c1cc(C(=N)N)cs1] 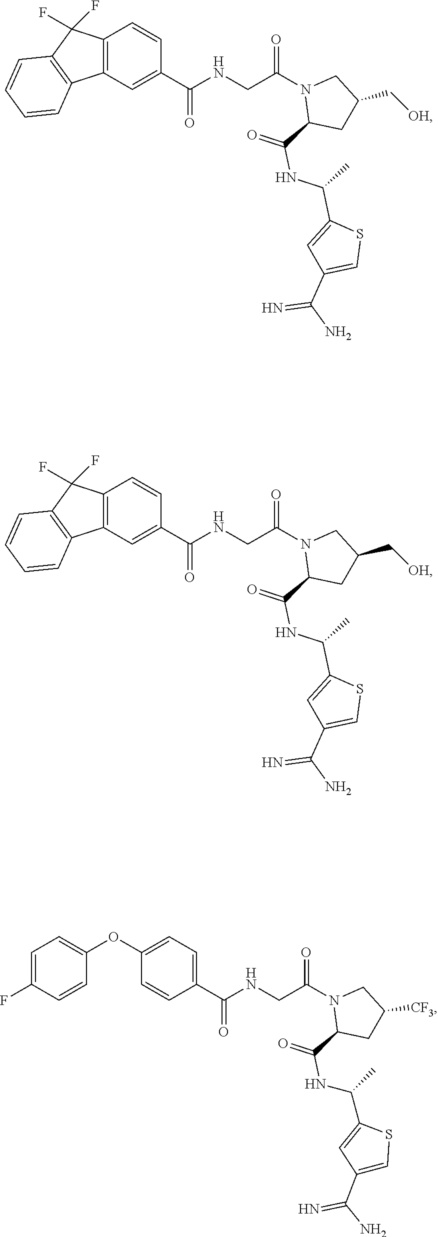 [SMILES: C[C@@H](NC(=O)[C@@H]1C[C@@H](CO)CN1C(=O)CNC(=O)c1ccc2c(c1)-c1ccccc1C2(F)F)c1cc(C(=N)N)cs1 ; C[C@@H](NC(=O)[C@@H]1C[C@H](CO)CN1C(=O)CNC(=O)c1ccc2c(c1)-c1ccccc1C2(F)F)c1cc(C(=N)N)cs1 ; C[C@@H](NC(=O)[C@@H]1C[C@@H](C(F)(F)F)CN1C(=O)CNC(=O)c1ccc(Oc2ccc(F)cc2)cc1)c1cc(C(=N)N)cs1] 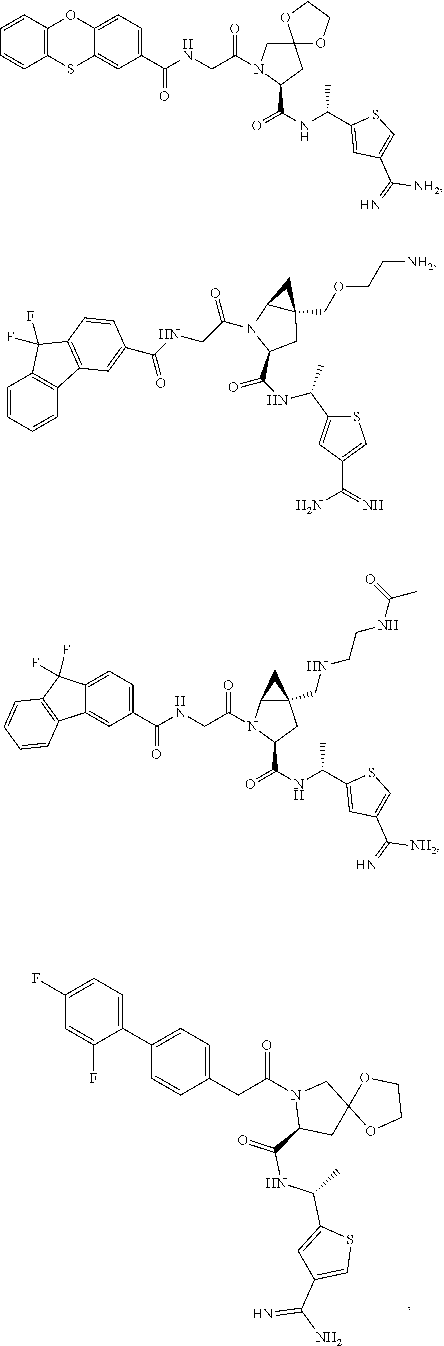 [SMILES: C[C@@H](NC(=O)[C@@H]1CC2(CN1C(=O)CNC(=O)c1ccc3c(c1)Sc1ccccc1O3)OCCO2)c1cc(C(=N)N)cs1 ; C[C@@H](NC(=O)[C@@H]1C[C@]2(COCCN)C[C@@H]2N1C(=O)CNC(=O)c1ccc2c(c1)-c1ccccc1C2(F)F)c1cc(C(=N)N)cs1 ; CC(=O)NCCNC[C@@]12C[C@@H]1N(C(=O)CNC(=O)c1ccc3c(c1)-c1ccccc1C3(F)F)[C@H](C(=O)N[C@H](C)c1cc(C(=N)N)cs1)C2 ; C[C@@H](NC(=O)[C@@H]1CC2(CN1C(=O)Cc1ccc(-c3ccc(F)cc3F)cc1)OCCO2)c1cc(C(=N)N)cs1] 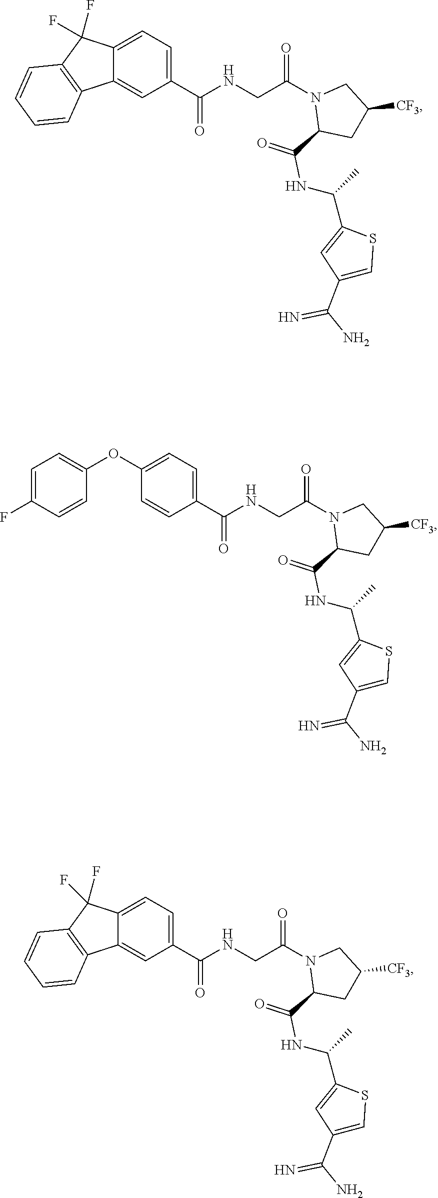 [SMILES: C[C@@H](NC(=O)[C@@H]1C[C@H](C(F)(F)F)CN1C(=O)CNC(=O)c1ccc2c(c1)-c1ccccc1C2(F)F)c1cc(C(=N)N)cs1 ; C[C@@H](NC(=O)[C@@H]1C[C@H](C(F)(F)F)CN1C(=O)CNC(=O)c1ccc(Oc2ccc(F)cc2)cc1)c1cc(C(=N)N)cs1 ; C[C@@H](NC(=O)[C@@H]1C[C@@H](C(F)(F)F)CN1C(=O)CNC(=O)c1ccc2c(c1)-c1ccccc1C2(F)F)c1cc(C(=N)N)cs1] 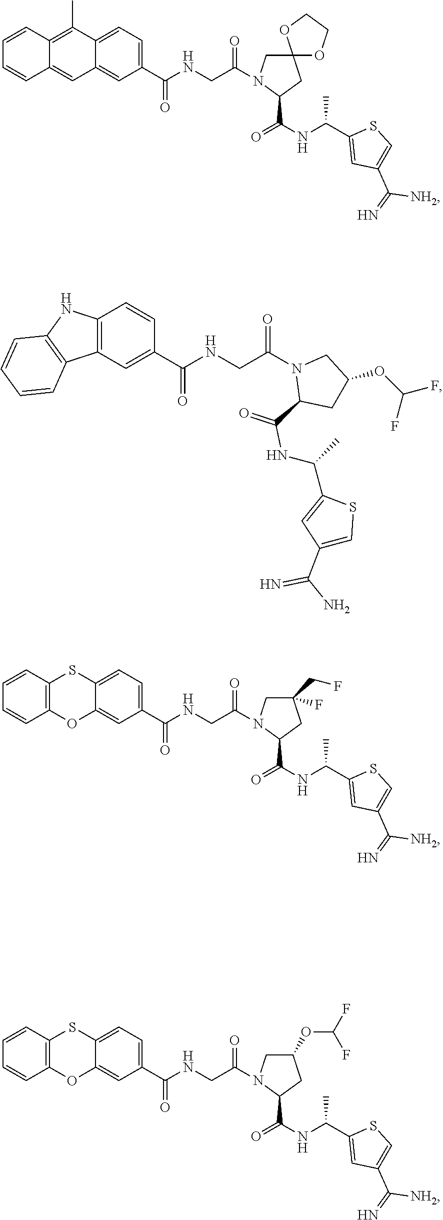 [SMILES: Cc1c2ccccc2cc2cc(C(=O)NCC(=O)N3CC4(C[C@H]3C(=O)N[C@H](C)c3cc(C(=N)N)cs3)OCCO4)ccc12 ; C[C@@H](NC(=O)[C@@H]1C[C@@H](OC(F)F)CN1C(=O)CNC(=O)c1ccc2[nH]c3ccccc3c2c1)c1cc(C(=N)N)cs1 ; C[C@@H](NC(=O)[C@@H]1C[C@](F)(CF)CN1C(=O)CNC(=O)c1ccc2c(c1)Oc1ccccc1S2)c1cc(C(=N)N)cs1 ; C[C@@H](NC(=O)[C@@H]1C[C@@H](OC(F)F)CN1C(=O)CNC(=O)c1ccc2c(c1)Oc1ccccc1S2)c1cc(C(=N)N)cs1] 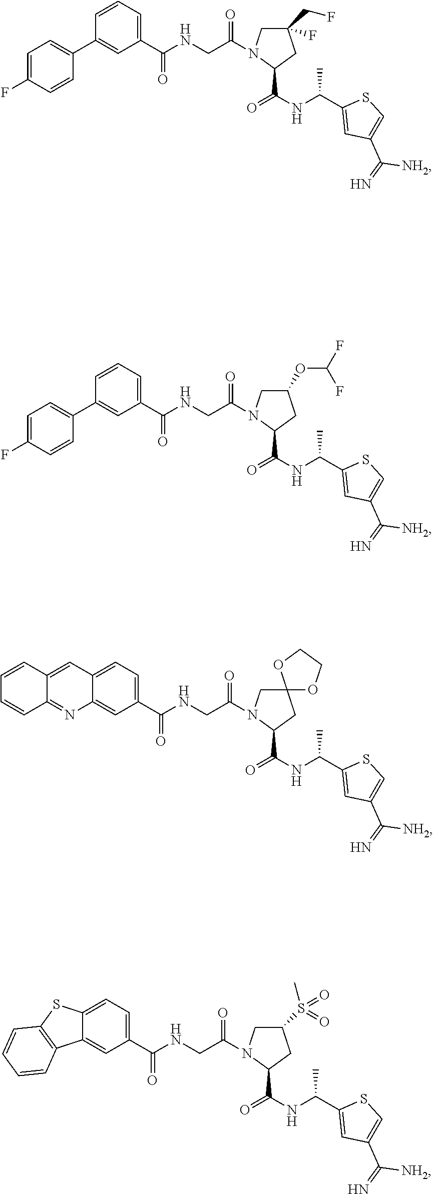 [SMILES: C[C@@H](NC(=O)[C@@H]1C[C@](F)(CF)CN1C(=O)CNC(=O)c1cccc(-c2ccc(F)cc2)c1)c1cc(C(=N)N)cs1 ; C[C@@H](NC(=O)[C@@H]1C[C@@H](OC(F)F)CN1C(=O)CNC(=O)c1cccc(-c2ccc(F)cc2)c1)c1cc(C(=N)N)cs1 ; C[C@@H](NC(=O)[C@@H]1CC2(CN1C(=O)CNC(=O)c1ccc3cc4ccccc4nc3c1)OCCO2)c1cc(C(=N)N)cs1 ; C[C@@H](NC(=O)[C@@H]1C[C@@H](S(C)(=O)=O)CN1C(=O)CNC(=O)c1ccc2sc3ccccc3c2c1)c1cc(C(=N)N)cs1] 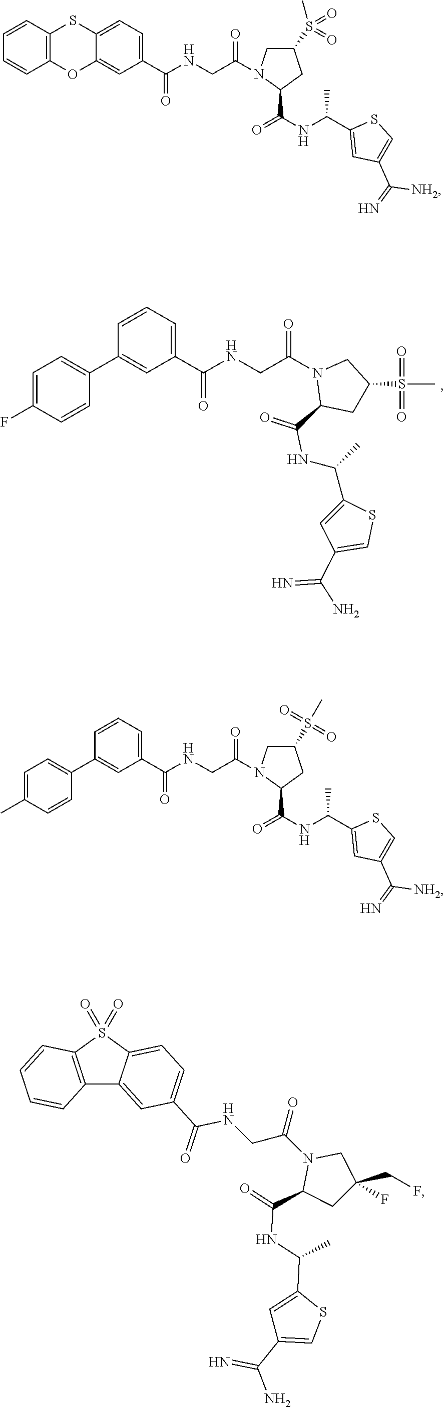 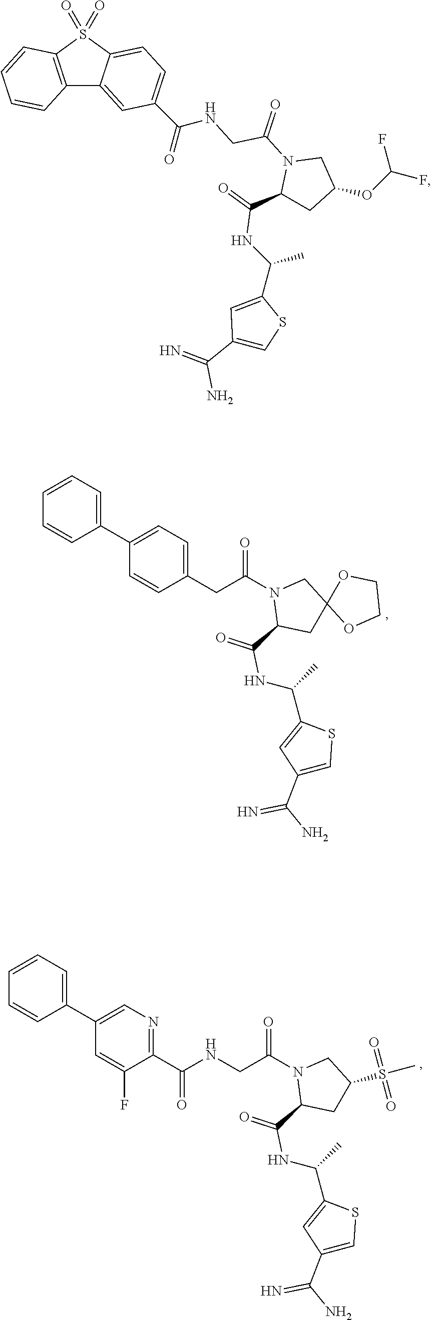 [SMILES: C[C@@H](NC(=O)[C@@H]1C[C@@H](OC(F)F)CN1C(=O)CNC(=O)c1ccc2c(c1)-c1ccccc1S2(=O)=O)c1cc(C(=N)N)cs1 ; C[C@@H](NC(=O)[C@@H]1CC2(CN1C(=O)Cc1ccc(-c3ccccc3)cc1)OCCO2)c1cc(C(=N)N)cs1 ; C[C@@H](NC(=O)[C@@H]1C[C@@H](S(C)(=O)=O)CN1C(=O)CNC(=O)c1ncc(-c2ccccc2)cc1F)c1cc(C(=N)N)cs1] 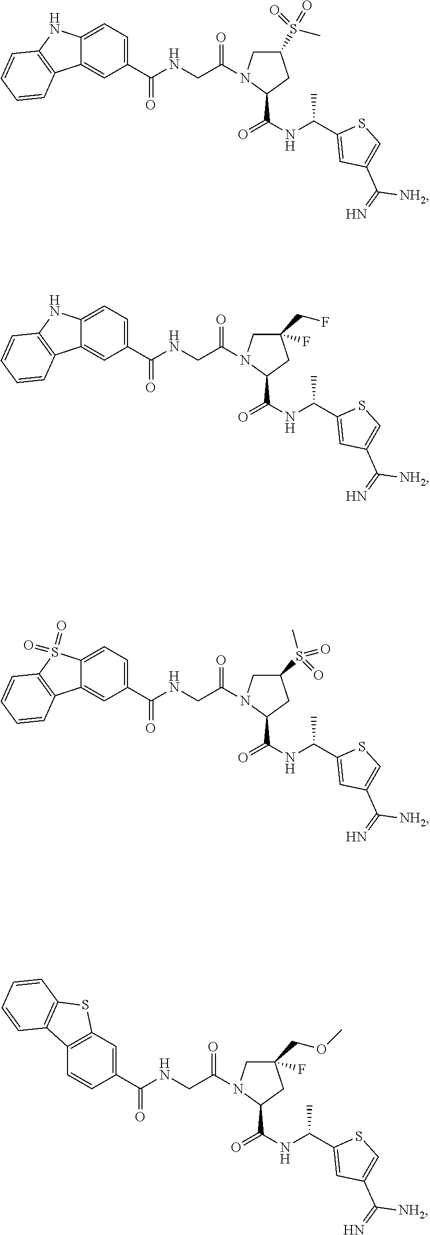 [SMILES: C[C@@H](NC(=O)[C@@H]1C[C@@H](S(C)(=O)=O)CN1C(=O)CNC(=O)c1ccc2[nH]c3ccccc3c2c1)c1cc(C(=N)N)cs1 ; C[C@@H](NC(=O)[C@@H]1C[C@](F)(CF)CN1C(=O)CNC(=O)c1ccc2[nH]c3ccccc3c2c1)c1cc(C(=N)N)cs1 ; C[C@@H](NC(=O)[C@@H]1C[C@H](S(C)(=O)=O)CN1C(=O)CNC(=O)c1ccc2c(c1)-c1ccccc1S2(=O)=O)c1cc(C(=N)N)cs1 ; COC[C@@]1(F)C[C@@H](C(=O)N[C@H](C)c2cc(C(=N)N)cs2)N(C(=O)CNC(=O)c2ccc3c(c2)sc2ccccc23)C1] 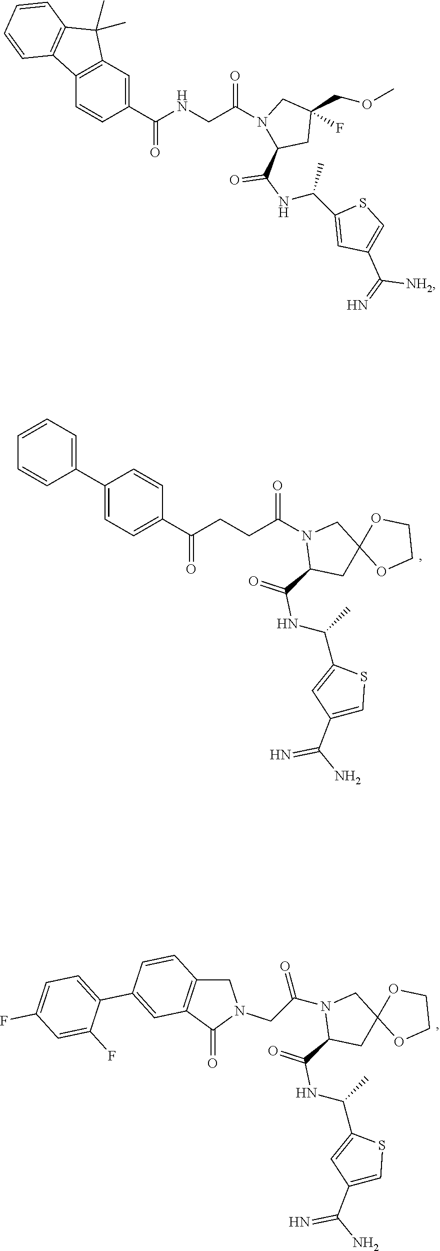 [SMILES: COC[C@@]1(F)C[C@@H](C(=O)N[C@H](C)c2cc(C(=N)N)cs2)N(C(=O)CNC(=O)c2ccc3c(c2)C(C)(C)c2ccccc2-3)C1 ; C[C@@H](NC(=O)[C@@H]1CC2(CN1C(=O)CCC(=O)c1ccc(-c3ccccc3)cc1)OCCO2)c1cc(C(=N)N)cs1 ; C[C@@H](NC(=O)[C@@H]1CC2(CN1C(=O)CN1Cc3ccc(-c4ccc(F)cc4F)cc3C1=O)OCCO2)c1cc(C(=N)N)cs1] 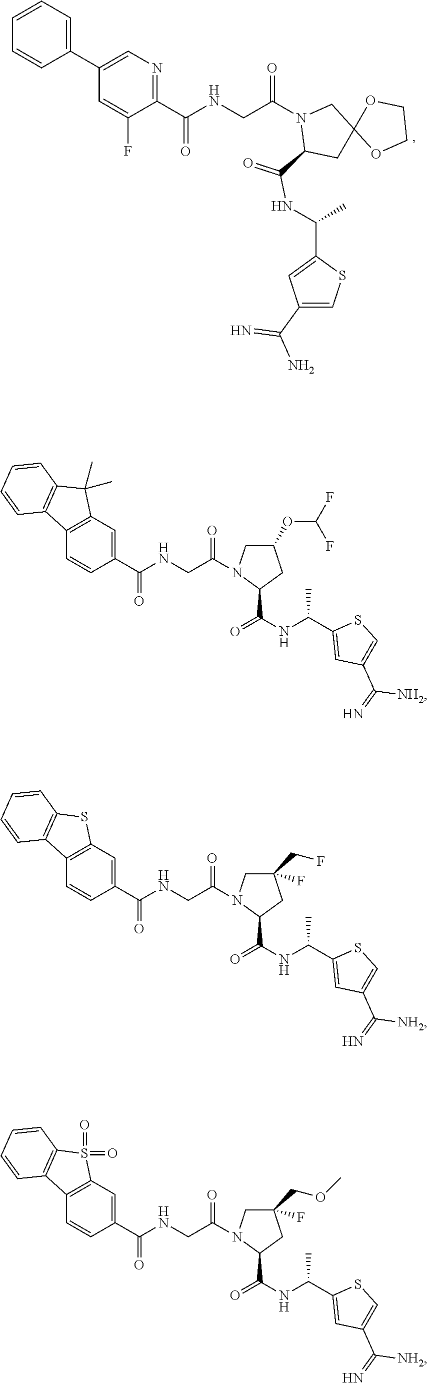 [SMILES: C[C@@H](NC(=O)[C@@H]1CC2(CN1C(=O)CNC(=O)c1ncc(-c3ccccc3)cc1F)OCCO2)c1cc(C(=N)N)cs1 ; C[C@@H](NC(=O)[C@@H]1C[C@@H](OC(F)F)CN1C(=O)CNC(=O)c1ccc2c(c1)C(C)(C)c1ccccc1-2)c1cc(C(=N)N)cs1 ; C[C@@H](NC(=O)[C@@H]1C[C@](F)(CF)CN1C(=O)CNC(=O)c1ccc2c(c1)sc1ccccc12)c1cc(C(=N)N)cs1 ; COC[C@@]1(F)C[C@@H](C(=O)N[C@H](C)c2cc(C(=N)N)cs2)N(C(=O)CNC(=O)c2ccc3c(c2)S(=O)(=O)c2ccccc2-3)C1] 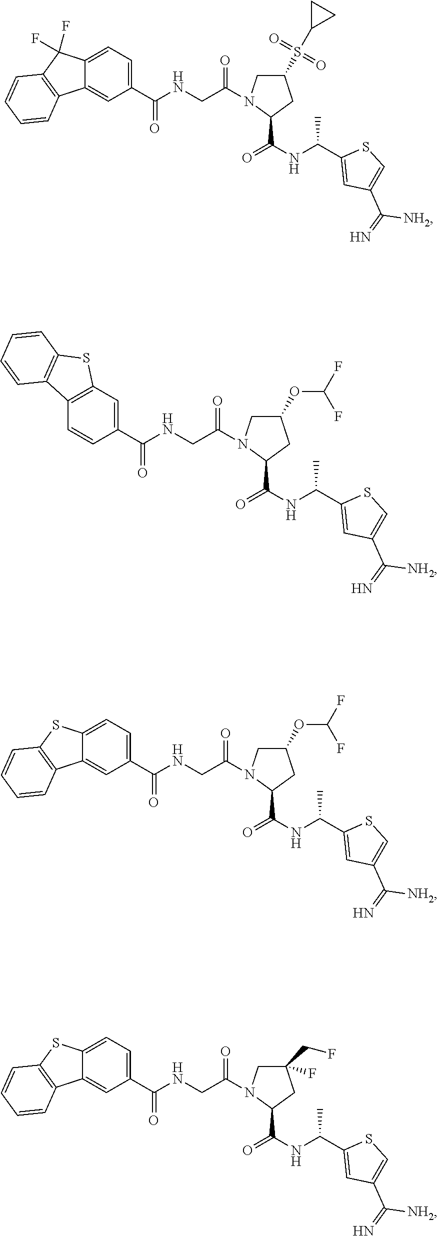 [SMILES: C[C@@H](NC(=O)[C@@H]1C[C@@H](S(=O)(=O)C2CC2)CN1C(=O)CNC(=O)c1ccc2c(c1)-c1ccccc1C2(F)F)c1cc(C(=N)N)cs1 ; C[C@@H](NC(=O)[C@@H]1C[C@@H](OC(F)F)CN1C(=O)CNC(=O)c1ccc2c(c1)sc1ccccc12)c1cc(C(=N)N)cs1 ; C[C@@H](NC(=O)[C@@H]1C[C@@H](OC(F)F)CN1C(=O)CNC(=O)c1ccc2sc3ccccc3c2c1)c1cc(C(=N)N)cs1 ; C[C@@H](NC(=O)[C@@H]1C[C@](F)(CF)CN1C(=O)CNC(=O)c1ccc2sc3ccccc3c2c1)c1cc(C(=N)N)cs1] 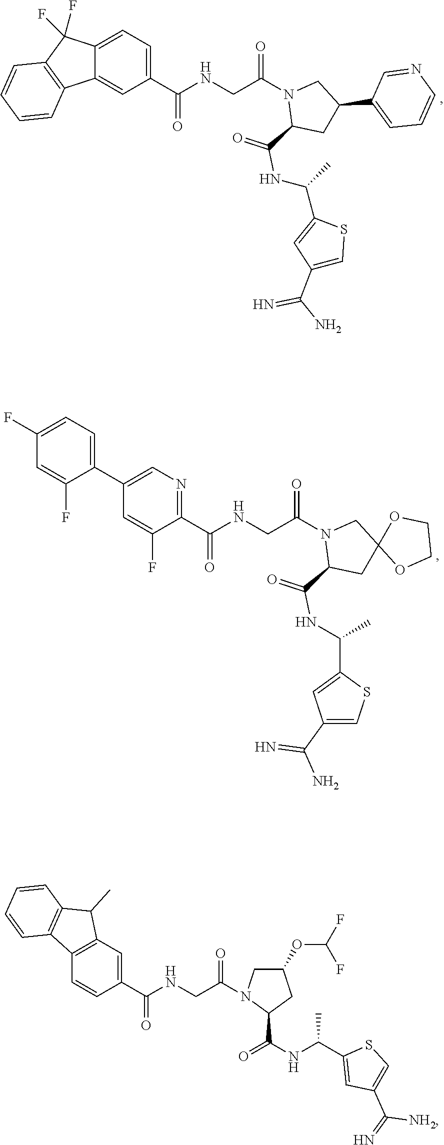 [SMILES: C[C@@H](NC(=O)[C@@H]1C[C@H](c2cccnc2)CN1C(=O)CNC(=O)c1ccc2c(c1)-c1ccccc1C2(F)F)c1cc(C(=N)N)cs1 ; C[C@@H](NC(=O)[C@@H]1CC2(CN1C(=O)CNC(=O)c1ncc(-c3ccc(F)cc3F)cc1F)OCCO2)c1cc(C(=N)N)cs1 ; CC1c2ccccc2-c2ccc(C(=O)NCC(=O)N3C[C@H](OC(F)F)C[C@H]3C(=O)N[C@H](C)c3cc(C(=N)N)cs3)cc21] 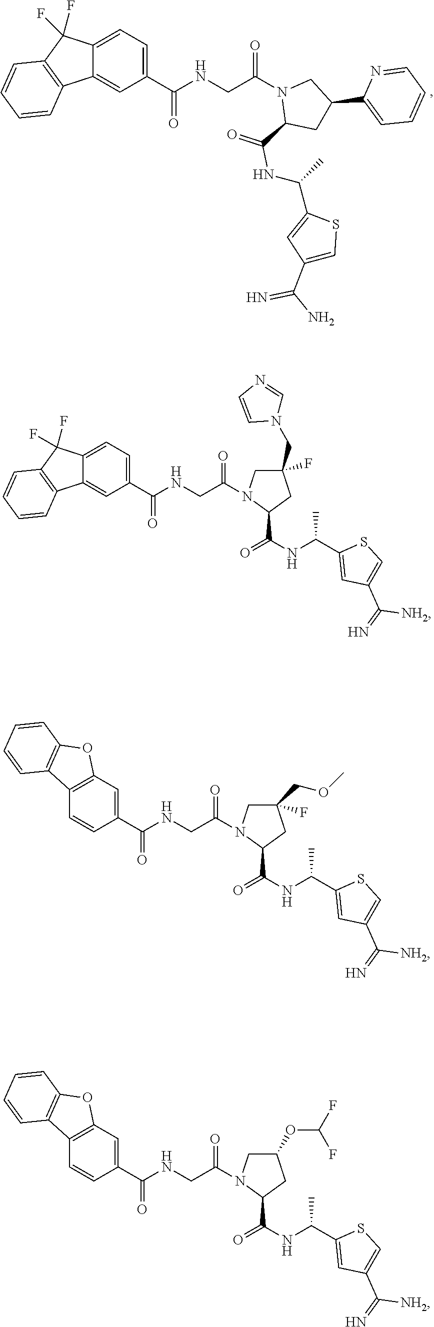 [SMILES: C[C@@H](NC(=O)[C@@H]1C[C@H](c2ccccn2)CN1C(=O)CNC(=O)c1ccc2c(c1)-c1ccccc1C2(F)F)c1cc(C(=N)N)cs1 ; C[C@@H](NC(=O)[C@@H]1C[C@](F)(Cn2ccnc2)CN1C(=O)CNC(=O)c1ccc2c(c1)-c1ccccc1C2(F)F)c1cc(C(=N)N)cs1 ; COC[C@@]1(F)C[C@@H](C(=O)N[C@H](C)c2cc(C(=N)N)cs2)N(C(=O)CNC(=O)c2ccc3c(c2)oc2ccccc23)C1 ; C[C@@H](NC(=O)[C@@H]1C[C@@H](OC(F)F)CN1C(=O)CNC(=O)c1ccc2c(c1)oc1ccccc12)c1cc(C(=N)N)cs1] 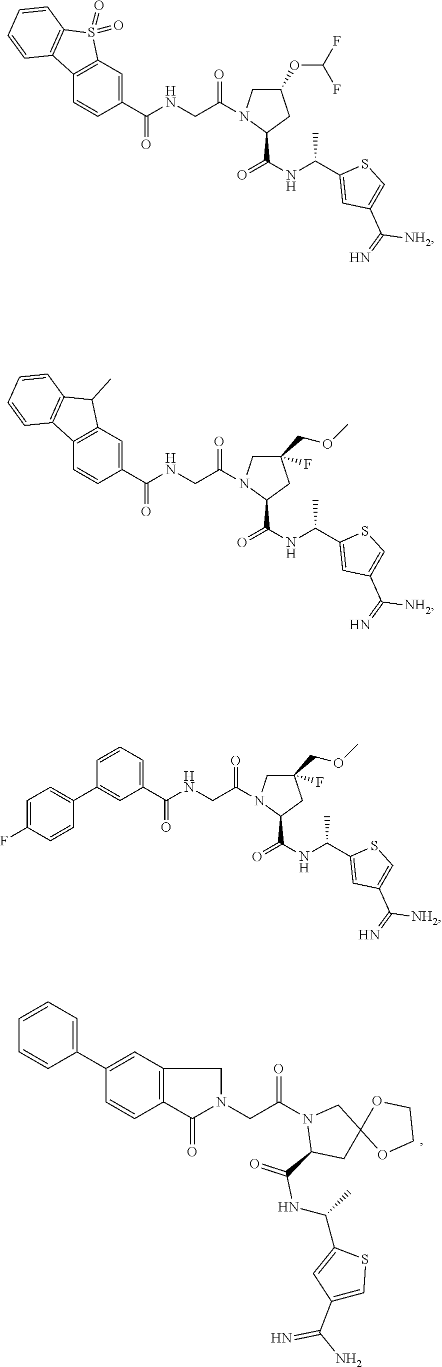 [SMILES: C[C@@H](NC(=O)[C@@H]1C[C@@H](OC(F)F)CN1C(=O)CNC(=O)c1ccc2c(c1)S(=O)(=O)c1ccccc1-2)c1cc(C(=N)N)cs1 ; COC[C@@]1(F)C[C@@H](C(=O)N[C@H](C)c2cc(C(=N)N)cs2)N(C(=O)CNC(=O)c2ccc3c(c2)C(C)c2ccccc2-3)C1 ; COC[C@@]1(F)C[C@@H](C(=O)N[C@H](C)c2cc(C(=N)N)cs2)N(C(=O)CNC(=O)c2cccc(-c3ccc(F)cc3)c2)C1 ; C[C@@H](NC(=O)[C@@H]1CC2(CN1C(=O)CN1Cc3cc(-c4ccccc4)ccc3C1=O)OCCO2)c1cc(C(=N)N)cs1] 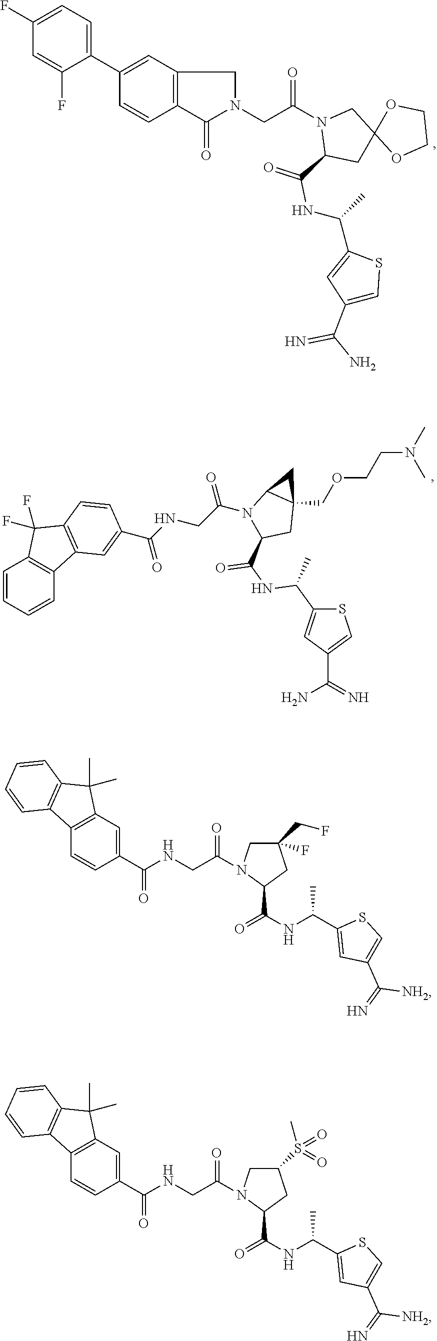 [SMILES: C[C@@H](NC(=O)[C@@H]1CC2(CN1C(=O)CN1Cc3cc(-c4ccc(F)cc4F)ccc3C1=O)OCCO2)c1cc(C(=N)N)cs1 ; C[C@@H](NC(=O)[C@@H]1C[C@]2(COCCN(C)C)C[C@@H]2N1C(=O)CNC(=O)c1ccc2c(c1)-c1ccccc1C2(F)F)c1cc(C(=N)N)cs1 ; C[C@@H](NC(=O)[C@@H]1C[C@](F)(CF)CN1C(=O)CNC(=O)c1ccc2c(c1)C(C)(C)c1ccccc1-2)c1cc(C(=N)N)cs1 ; C[C@@H](NC(=O)[C@@H]1C[C@@H](S(C)(=O)=O)CN1C(=O)CNC(=O)c1ccc2c(c1)C(C)(C)c1ccccc1-2)c1cc(C(=N)N)cs1] 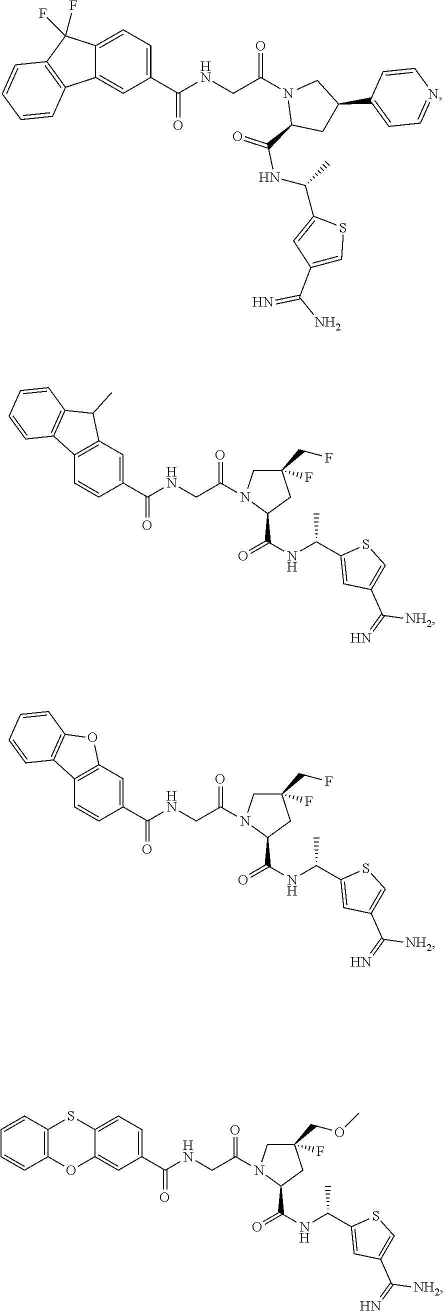 [SMILES: C[C@@H](NC(=O)[C@@H]1C[C@H](c2ccncc2)CN1C(=O)CNC(=O)c1ccc2c(c1)-c1ccccc1C2(F)F)c1cc(C(=N)N)cs1 ; CC1c2ccccc2-c2ccc(C(=O)NCC(=O)N3C[C@@](F)(CF)C[C@H]3C(=O)N[C@H](C)c3cc(C(=N)N)cs3)cc21 ; C[C@@H](NC(=O)[C@@H]1C[C@](F)(CF)CN1C(=O)CNC(=O)c1ccc2c(c1)oc1ccccc12)c1cc(C(=N)N)cs1 ; COC[C@@]1(F)C[C@@H](C(=O)N[C@H](C)c2cc(C(=N)N)cs2)N(C(=O)CNC(=O)c2ccc3c(c2)Oc2ccccc2S3)C1] 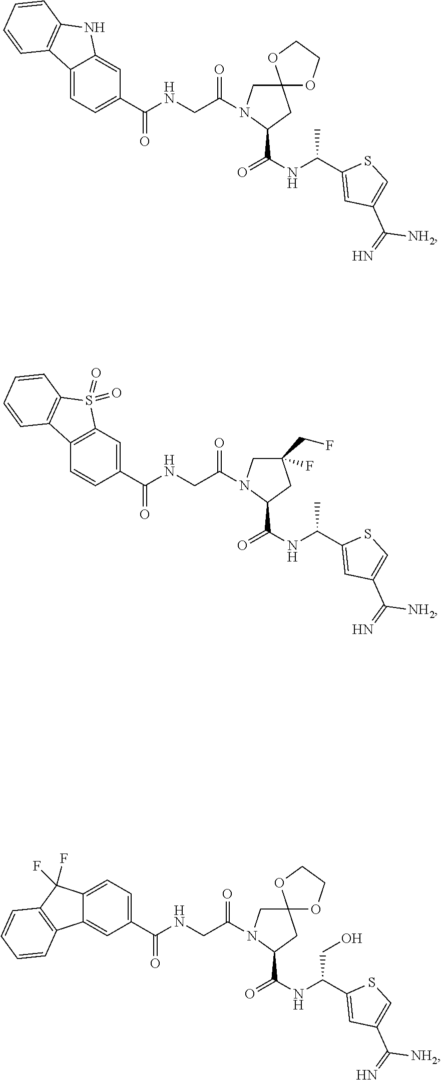 [SMILES: C[C@@H](NC(=O)[C@@H]1CC2(CN1C(=O)CNC(=O)c1ccc3c(c1)[nH]c1ccccc13)OCCO2)c1cc(C(=N)N)cs1 ; C[C@@H](NC(=O)[C@@H]1C[C@](F)(CF)CN1C(=O)CNC(=O)c1ccc2c(c1)S(=O)(=O)c1ccccc1-2)c1cc(C(=N)N)cs1 ; N=C(N)c1csc([C@@H](CO)NC(=O)[C@@H]2CC3(CN2C(=O)CNC(=O)c2ccc4c(c2)-c2ccccc2C4(F)F)OCCO3)c1] 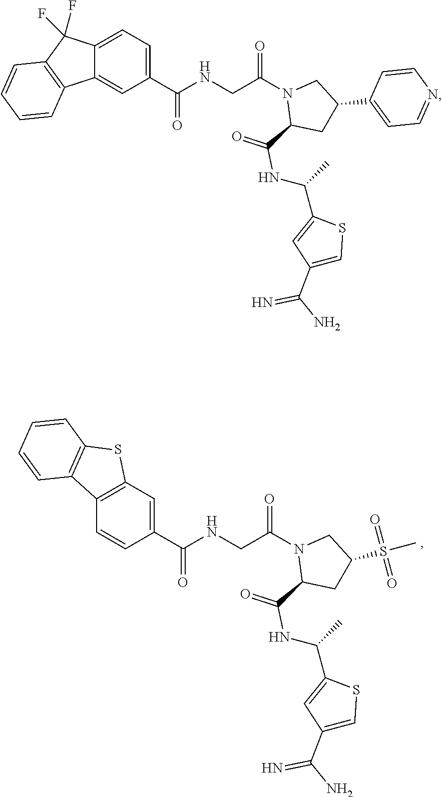 [SMILES: C[C@@H](NC(=O)[C@@H]1C[C@@H](c2ccncc2)CN1C(=O)CNC(=O)c1ccc2c(c1)-c1ccccc1C2(F)F)c1cc(C(=N)N)cs1 ; C[C@@H](NC(=O)[C@@H]1C[C@@H](S(C)(=O)=O)CN1C(=O)CNC(=O)c1ccc2c(c1)sc1ccccc12)c1cc(C(=N)N)cs1] 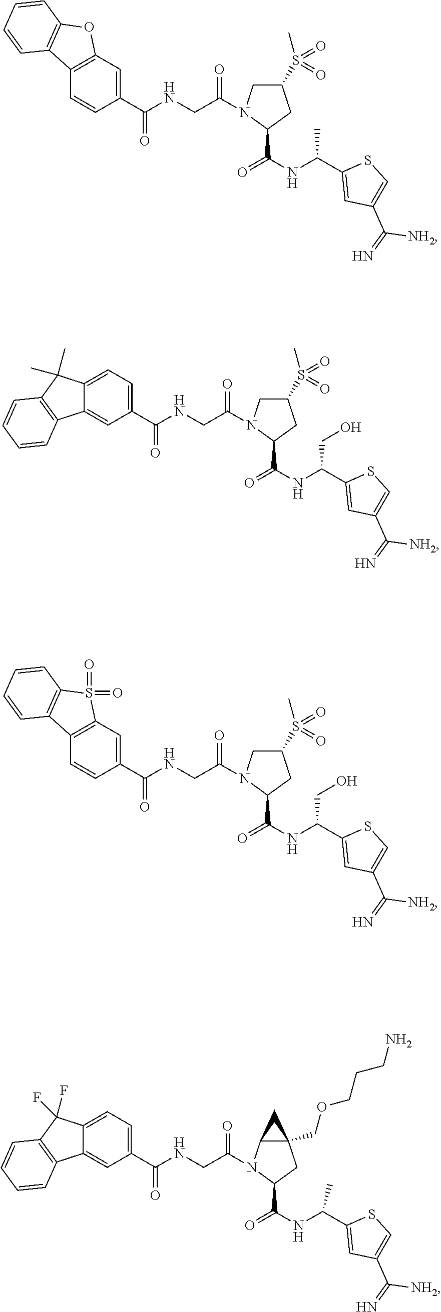 [SMILES: C[C@@H](NC(=O)[C@@H]1C[C@@H](S(C)(=O)=O)CN1C(=O)CNC(=O)c1ccc2c(c1)oc1ccccc12)c1cc(C(=N)N)cs1 ; CC1(C)c2ccccc2-c2cc(C(=O)NCC(=O)N3C[C@H](S(C)(=O)=O)C[C@H]3C(=O)N[C@H](CO)c3cc(C(=N)N)cs3)ccc21 ; CS(=O)(=O)[C@@H]1C[C@@H](C(=O)N[C@H](CO)c2cc(C(=N)N)cs2)N(C(=O)CNC(=O)c2ccc3c(c2)S(=O)(=O)c2ccccc2-3)C1 ; C[C@@H](NC(=O)[C@@H]1C[C@]2(COCCCN)C[C@@H]2N1C(=O)CNC(=O)c1ccc2c(c1)-c1ccccc1C2(F)F)c1cc(C(=N)N)cs1] 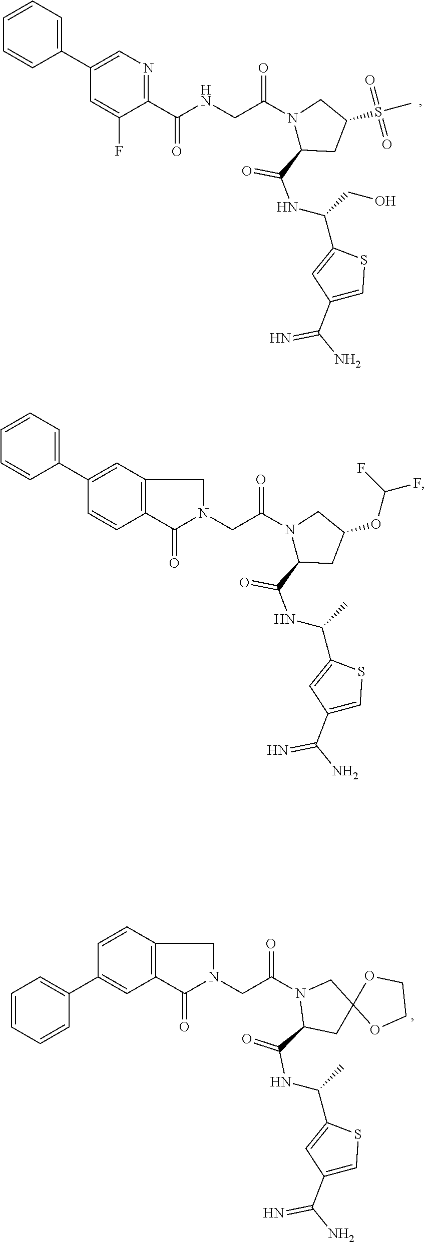 [SMILES: CS(=O)(=O)[C@@H]1C[C@@H](C(=O)N[C@H](CO)c2cc(C(=N)N)cs2)N(C(=O)CNC(=O)c2ncc(-c3ccccc3)cc2F)C1 ; C[C@@H](NC(=O)[C@@H]1C[C@@H](OC(F)F)CN1C(=O)CN1Cc2cc(-c3ccccc3)ccc2C1=O)c1cc(C(=N)N)cs1 ; C[C@@H](NC(=O)[C@@H]1CC2(CN1C(=O)CN1Cc3ccc(-c4ccccc4)cc3C1=O)OCCO2)c1cc(C(=N)N)cs1] 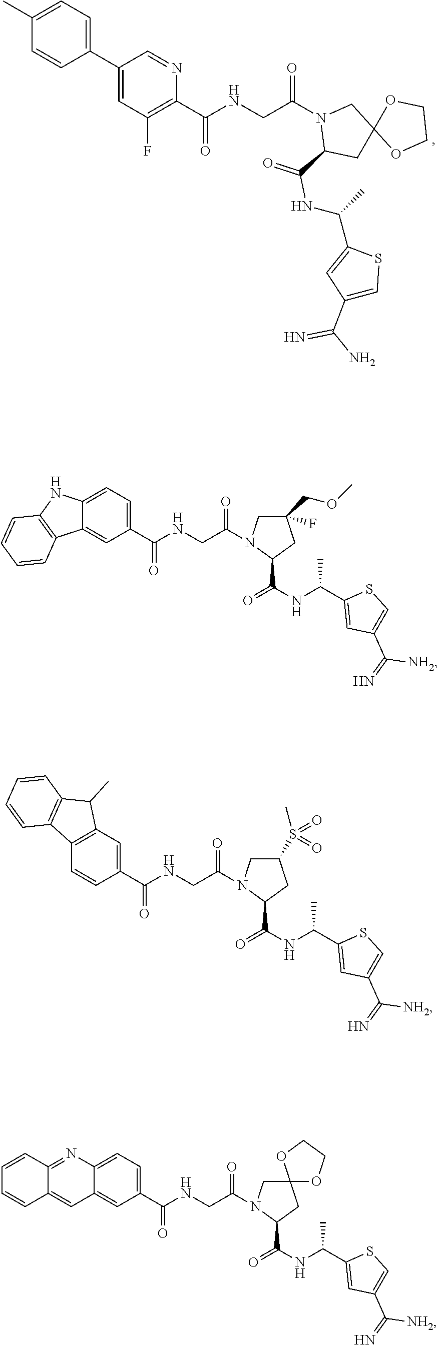 [SMILES: Cc1ccc(-c2cnc(C(=O)NCC(=O)N3CC4(C[C@H]3C(=O)N[C@H](C)c3cc(C(=N)N)cs3)OCCO4)c(F)c2)cc1 ; COC[C@@]1(F)C[C@@H](C(=O)N[C@H](C)c2cc(C(=N)N)cs2)N(C(=O)CNC(=O)c2ccc3[nH]c4ccccc4c3c2)C1 ; CC1c2ccccc2-c2ccc(C(=O)NCC(=O)N3C[C@H](S(C)(=O)=O)C[C@H]3C(=O)N[C@H](C)c3cc(C(=N)N)cs3)cc21 ; C[C@@H](NC(=O)[C@@H]1CC2(CN1C(=O)CNC(=O)c1ccc3nc4ccccc4cc3c1)OCCO2)c1cc(C(=N)N)cs1] 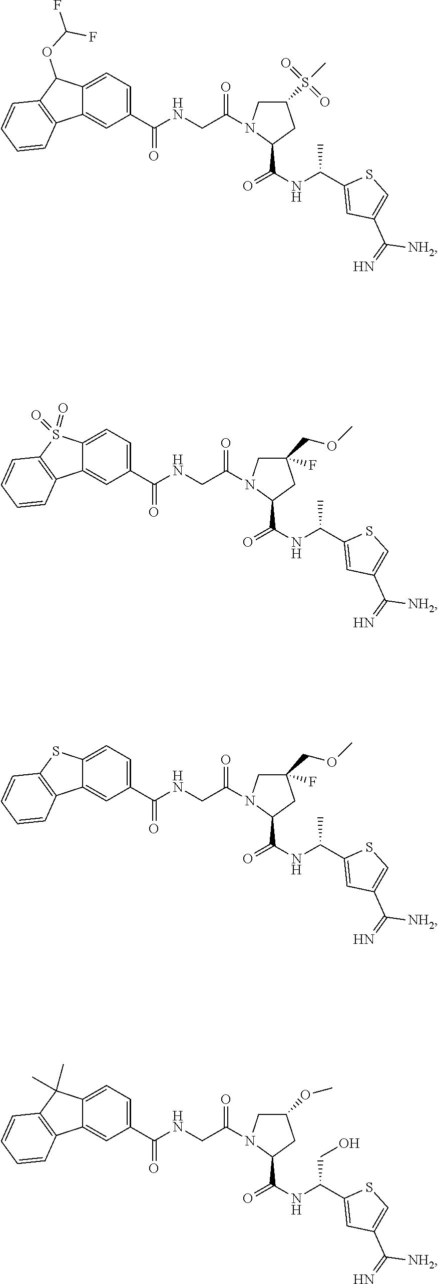 [SMILES: C[C@@H](NC(=O)[C@@H]1C[C@@H](S(C)(=O)=O)CN1C(=O)CNC(=O)c1ccc2c(c1)-c1ccccc1C2OC(F)F)c1cc(C(=N)N)cs1 ; COC[C@@]1(F)C[C@@H](C(=O)N[C@H](C)c2cc(C(=N)N)cs2)N(C(=O)CNC(=O)c2ccc3c(c2)-c2ccccc2S3(=O)=O)C1 ; COC[C@@]1(F)C[C@@H](C(=O)N[C@H](C)c2cc(C(=N)N)cs2)N(C(=O)CNC(=O)c2ccc3sc4ccccc4c3c2)C1 ; CO[C@@H]1C[C@@H](C(=O)N[C@H](CO)c2cc(C(=N)N)cs2)N(C(=O)CNC(=O)c2ccc3c(c2)-c2ccccc2C3(C)C)C1] 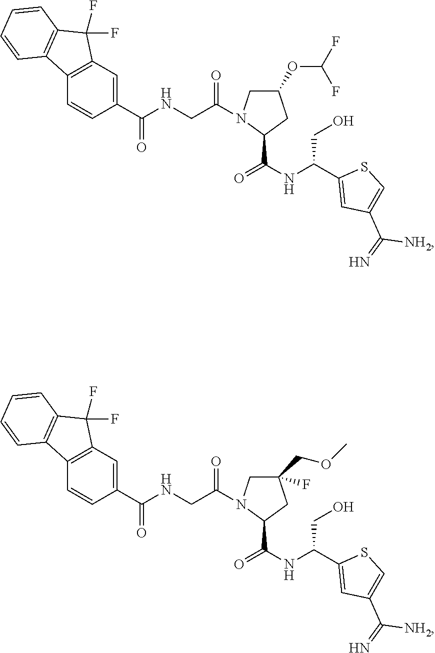 [SMILES: N=C(N)c1csc([C@@H](CO)NC(=O)[C@@H]2C[C@@H](OC(F)F)CN2C(=O)CNC(=O)c2ccc3c(c2)C(F)(F)c2ccccc2-3)c1 ; COC[C@@]1(F)C[C@@H](C(=O)N[C@H](CO)c2cc(C(=N)N)cs2)N(C(=O)CNC(=O)c2ccc3c(c2)C(F)(F)c2ccccc2-3)C1] 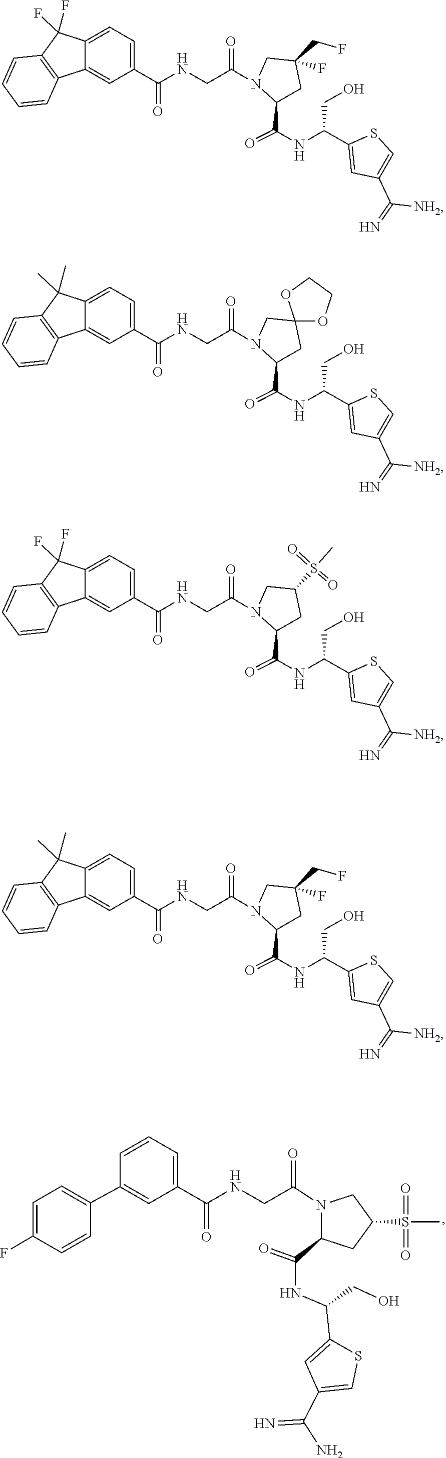 [SMILES: N=C(N)c1csc([C@@H](CO)NC(=O)[C@@H]2C[C@](F)(CF)CN2C(=O)CNC(=O)c2ccc3c(c2)-c2ccccc2C3(F)F)c1 ; CC1(C)c2ccccc2-c2cc(C(=O)NCC(=O)N3CC4(C[C@H]3C(=O)N[C@H](CO)c3cc(C(=N)N)cs3)OCCO4)ccc21 ; CS(=O)(=O)[C@@H]1C[C@@H](C(=O)N[C@H](CO)c2cc(C(=N)N)cs2)N(C(=O)CNC(=O)c2ccc3c(c2)-c2ccccc2C3(F)F)C1 ; CC1(C)c2ccccc2-c2cc(C(=O)NCC(=O)N3C[C@@](F)(CF)C[C@H]3C(=O)N[C@H](CO)c3cc(C(=N)N)cs3)ccc21 ; CS(=O)(=O)[C@@H]1C[C@@H](C(=O)N[C@H](CO)c2cc(C(=N)N)cs2)N(C(=O)CNC(=O)c2cccc(-c3ccc(F)cc3)c2)C1] 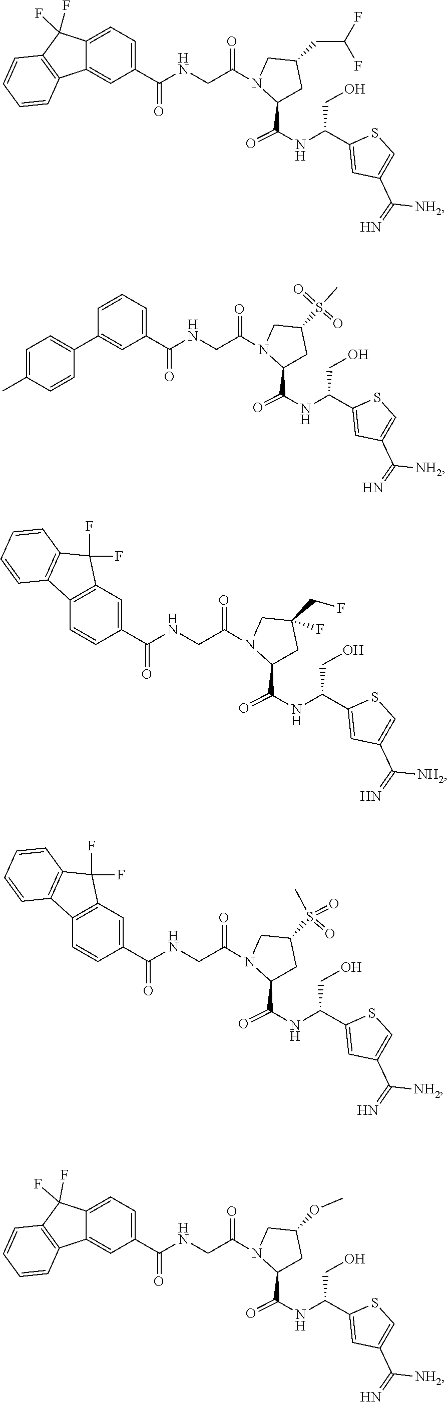 [SMILES: N=C(N)c1csc([C@@H](CO)NC(=O)[C@@H]2C[C@@H](CC(F)F)CN2C(=O)CNC(=O)c2ccc3c(c2)-c2ccccc2C3(F)F)c1 ; Cc1ccc(-c2cccc(C(=O)NCC(=O)N3C[C@H](S(C)(=O)=O)C[C@H]3C(=O)N[C@H](CO)c3cc(C(=N)N)cs3)c2)cc1 ; N=C(N)c1csc([C@@H](CO)NC(=O)[C@@H]2C[C@](F)(CF)CN2C(=O)CNC(=O)c2ccc3c(c2)C(F)(F)c2ccccc2-3)c1 ; CS(=O)(=O)[C@@H]1C[C@@H](C(=O)N[C@H](CO)c2cc(C(=N)N)cs2)N(C(=O)CNC(=O)c2ccc3c(c2)C(F)(F)c2ccccc2-3)C1 ; CO[C@@H]1C[C@@H](C(=O)N[C@H](CO)c2cc(C(=N)N)cs2)N(C(=O)CNC(=O)c2ccc3c(c2)-c2ccccc2C3(F)F)C1] 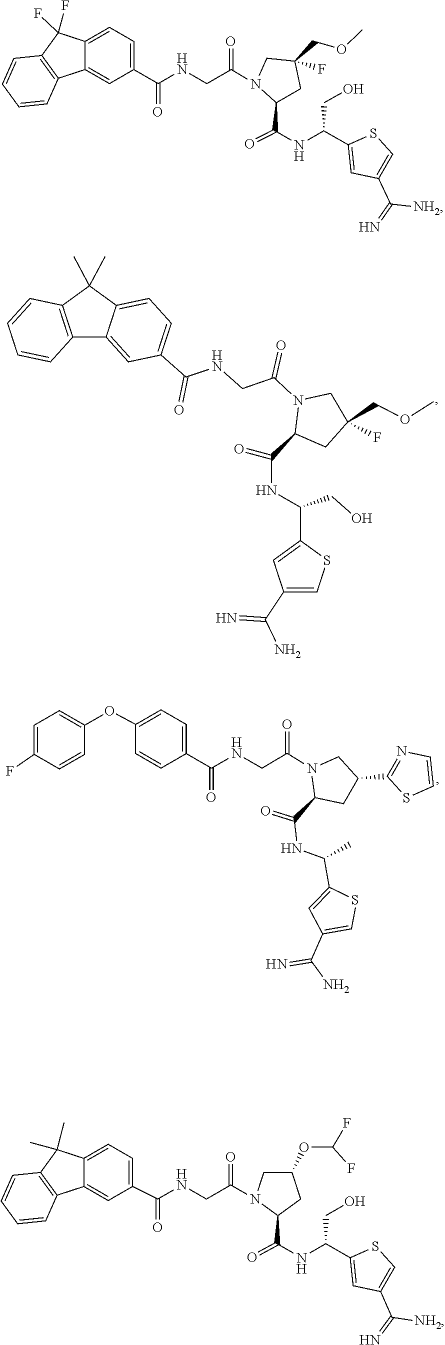 [SMILES: COC[C@@]1(F)C[C@@H](C(=O)N[C@H](CO)c2cc(C(=N)N)cs2)N(C(=O)CNC(=O)c2ccc3c(c2)-c2ccccc2C3(F)F)C1 ; COC[C@@]1(F)C[C@@H](C(=O)N[C@H](CO)c2cc(C(=N)N)cs2)N(C(=O)CNC(=O)c2ccc3c(c2)-c2ccccc2C3(C)C)C1 ; C[C@@H](NC(=O)[C@@H]1C[C@@H](c2nccs2)CN1C(=O)CNC(=O)c1ccc(Oc2ccc(F)cc2)cc1)c1cc(C(=N)N)cs1 ; CC1(C)c2ccccc2-c2cc(C(=O)NCC(=O)N3C[C@H](OC(F)F)C[C@H]3C(=O)N[C@H](CO)c3cc(C(=N)N)cs3)ccc21] 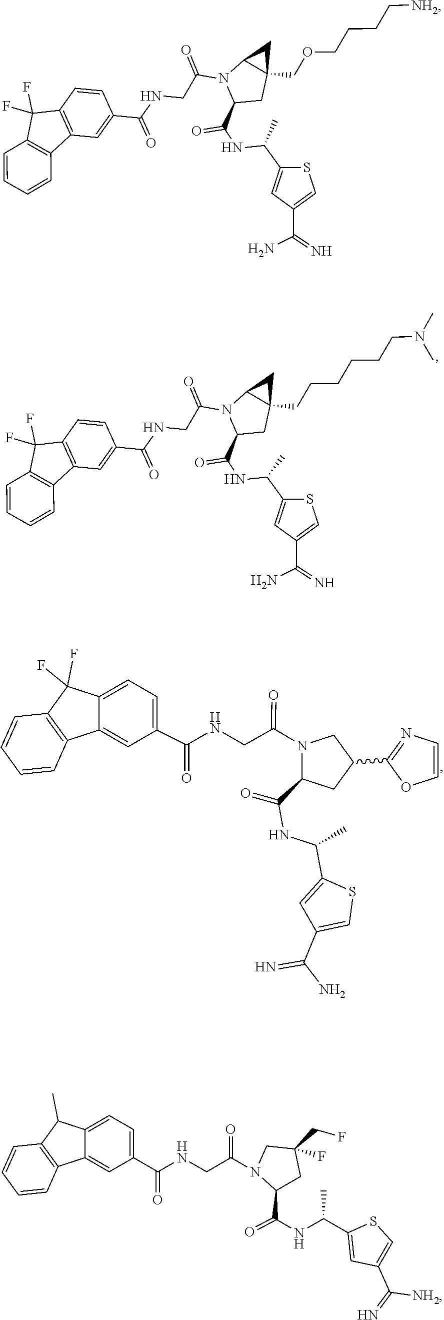 [SMILES: C[C@@H](NC(=O)[C@@H]1C[C@]2(COCCCCN)C[C@@H]2N1C(=O)CNC(=O)c1ccc2c(c1)-c1ccccc1C2(F)F)c1cc(C(=N)N)cs1 ; C[C@@H](NC(=O)[C@@H]1C[C@]2(CCCCCCN(C)C)C[C@@H]2N1C(=O)CNC(=O)c1ccc2c(c1)-c1ccccc1C2(F)F)c1cc(C(=N)N)cs1 ; C[C@@H](NC(=O)[C@@H]1CC(c2ncco2)CN1C(=O)CNC(=O)c1ccc2c(c1)-c1ccccc1C2(F)F)c1cc(C(=N)N)cs1 ; CC1c2ccccc2-c2cc(C(=O)NCC(=O)N3C[C@@](F)(CF)C[C@H]3C(=O)N[C@H](C)c3cc(C(=N)N)cs3)ccc21] 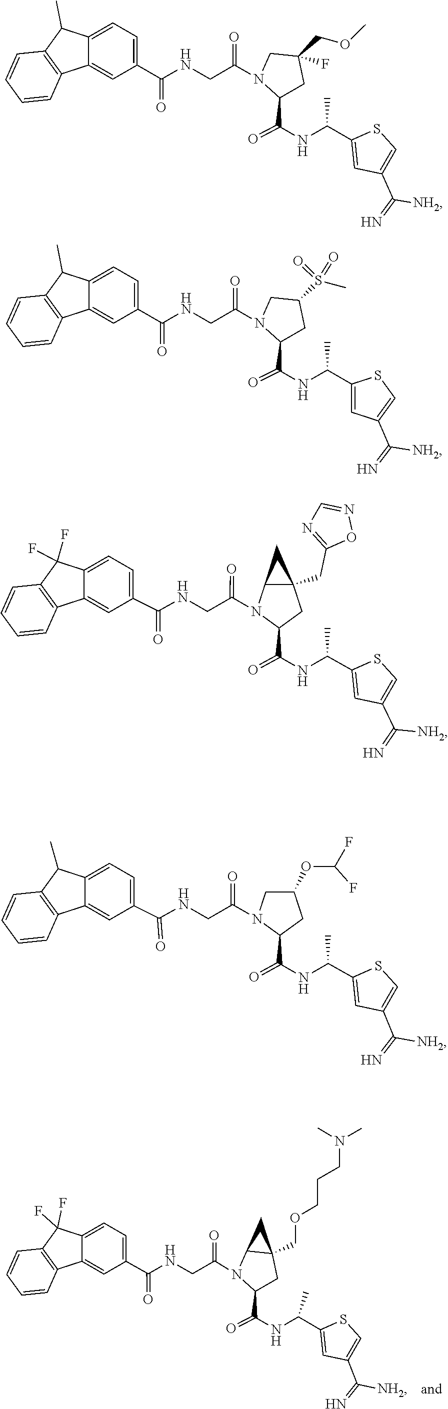 [SMILES: COC[C@@]1(F)C[C@@H](C(=O)N[C@H](C)c2cc(C(=N)N)cs2)N(C(=O)CNC(=O)c2ccc3c(c2)-c2ccccc2C3C)C1 ; CC1c2ccccc2-c2cc(C(=O)NCC(=O)N3C[C@H](S(C)(=O)=O)C[C@H]3C(=O)N[C@H](C)c3cc(C(=N)N)cs3)ccc21 ; C[C@@H](NC(=O)[C@@H]1C[C@]2(Cc3ncno3)C[C@@H]2N1C(=O)CNC(=O)c1ccc2c(c1)-c1ccccc1C2(F)F)c1cc(C(=N)N)cs1 ; CC1c2ccccc2-c2cc(C(=O)NCC(=O)N3C[C@H](OC(F)F)C[C@H]3C(=O)N[C@H](C)c3cc(C(=N)N)cs3)ccc21 ; C[C@@H](NC(=O)[C@@H]1C[C@]2(COCCCN(C)C)C[C@@H]2N1C(=O)CNC(=O)c1ccc2c(c1)-c1ccccc1C2(F)F)c1cc(C(=N)N)cs1] 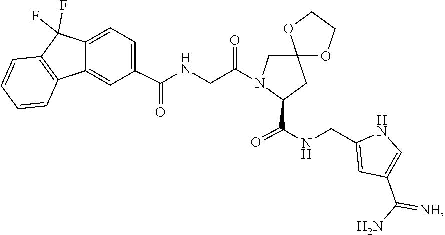 [SMILES: N=C(N)c1c[nH]c(CNC(=O)[C@@H]2CC3(CN2C(=O)CNC(=O)c2ccc4c(c2)-c2ccccc2C4(F)F)OCCO3)c1]
or a pharmaceutically acceptable salt thereof.

119. (canceled)

120. A pharmaceutical composition comprising a compound of claim 1 or a pharmaceutically acceptable salt thereof and a pharmaceutically acceptable excipient.

121. A method of treating a complement C1 esterase (C1s) mediated disorder, comprising administering to a human subject in need thereof a therapeutically effective amount of a compound of claim 1 or a pharmaceutically acceptable salt thereof.

122-123. (canceled)
